(19) United States
(12) Patent Application Publication
(10) Pub. No.: US 2023/0133132 A1
(43) Pub. Date: May 4, 2023

(54) NAMPT MODULATORS

(71) Applicant: Cytokinetics, Inc., South San Francisco, CA (US)

(72) Inventors: Antonio ROMERO, San Mateo, CA (US); Aroop CHANDRA, South San Francisco, CA (US); Christopher Edward EVANS, Pacifica, CA (US); Minxing SHEN, San Bruno, CA (US)

(21) Appl. No.: 17/798,296

(22) PCT Filed: Feb. 5, 2021
(86) PCT No.: PCT/US2021/016948

§ 371 Date: Aug. 8, 2022

Related U.S. Application Data
(60) Provisional application No. 62/971,838, filed on Feb. 7, 2020.

(51) Int. Cl. C07D 295/073; C07D 211/60; C07D 233/64; C07D 231/12; C07C 275/42; C07C 275/40; C07D 487/04; C07D 241/12; C07D 241/38; C07D 207/08; C07D 207/06; C07D 307/16; C07D 309/14; C07D 487/08; C07D 305/08; C07D 335/02; C07D 205/04; C07D 491/107; C07D 333/08; C07D 331/04; C07D 471/08; C07D 487/10; C07D 271/10; C07D 261/08; C07D 401/04; C07D 471/10; C07D 405/04; C07D 209/52; C07D 401/06; C07D 403/06; C07D 243/08; C07D 237/08; C07D 275/02; C07D 305/14; C07D 497/10; C07D 498/10; C07D 263/30; C07D 491/08; C07D 279/12; C07D 249/08

(52) U.S. Cl. CPC C07D 295/073; C07D 211/60; C07D 233/64; C07D 231/12; C07C 275/42; C07C 275/40; C07D 487/04; C07D 241/12; C07D 241/38; C07D 207/08; C07D 207/06; C07D 307/16; C07D 309/14; C07D 487/08; C07D 305/08; C07D 335/02; C07D 205/04; C07D 491/107; C07D 333/08; C07D 331/04; C07D 471/08; C07D 487/10; C07D 271/10; C07D 261/08; C07D 401/04; C07D 471/10; C07D 405/04; C07D 209/52; C07D 401/06; C07D 403/06; C07D 243/08; C07D 237/08; C07D 275/02; C07D 305/14; C07D 497/10; C07D 498/10; C07D 263/30; C07D 491/08; C07D 279/12; C07D 249/08; C07B 2200/05

(57) ABSTRACT

Provided are compounds of Formula (II) or a pharmaceutically acceptable salt thereof, wherein R¹, R², R³, R⁴, R⁵, R⁶, and p are as defined herein. Also provided is a pharmaceutically acceptable composition comprising a compound of Formula (II), or a pharmaceutically acceptable salt thereof. Also provided are methods of using a compound of Formula (II), or a pharmaceutically acceptable salt thereof.

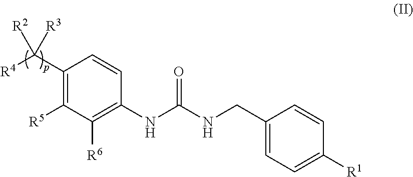

### CROSS-REFERENCE TO RELATED APPLICATIONS

This application claims priority to and benefit of U.S. Provisional Patent Application No. 62/971,838, filed Feb. 7, 2020, the disclosure of which is hereby incorporated herein by reference in its entirety.

### FIELD

Provided herein are phenyl urea compounds, pharmaceutical compositions comprising such compounds, and methods of treating various diseases and conditions mediated by nicotinamide phosphoribosyltransferase (NAMPT) with such compounds.

### BACKGROUND

The present disclosure relates to the use of modulators of nicotinamide phosphoribosyltransferase (NAMPT) and derivatives thereof, as well as enhancers or inducers of NAMPT expression, NAMPT activity or NAMPT-mediated signaling for preventing or treating a variety of pathological conditions.

Nicotinamide adenine dinucleotide (NAD+) is an essential coenzyme (enzyme cofactor) involved in fundamental biological processes of both catabolic and anabolic metabolism. As a coenzyme, NAD is associated with many oxidative enzymes (typically dehydrogenases) involved in energy metabolism, serving as a universal electron carrier. NAD exists in cells in the oxidized state (NAD+ and NADP+), and the reduced state (NADH and NADPH), acting as a chemical means to capture and transfer free energy from oxidative processes in catabolism, or to provide small packets of energy to build macromolecules in anabolism. NADH produced from the oxidation of carbohydrates, lipids, and amino acids provides reducing equivalents to the electron transport chain of mitochondria, ultimately driving the synthesis of ATP in oxidative phosphorylation.

More than 200 enzymes use either NAD+ or NADP+ as a coenzyme, and the enzymatic functions are not limited to energy metabolism. It is now appreciated that NAD+ plays a role in regulating diverse functions, including mitochondrial function, respiratory capacity, and biogenesis, mitochondrial-nuclear signaling. Further, it controls cell signaling, gene expression, DNA repair, hematopoiesis, immune function, the unfolded protein response, and autophagy. Furthermore, NAD is anti-inflammatory and is the precursor for NADPH, which is the primary source of reducing power for combating oxidative stress. A large body of literature indicates that boosting NAD levels is an effective strategy to either prevent or ameliorate a wide variety of disease states (Stromland et al., Biochem Soc Trans. 2019, 47(1):119-130; Ralto et al., Nat Rev Nephrol. 2019; Fang et al., Trends Mol Med. 2017, 23(10):899-916; Yoshino et al., Cell Metab. 2011, 14(4):528-36; Yang and Sauve, Biochim Biophys Acta. 2016, 1864:1787-1800; Verdin, Science. 2015, 350(6265):1208-13).

Levels of NAD+ and NADP+-associated enzymes play important roles in normal physiology and are altered under various disease and stress conditions including aging. Cellular NAD+ levels decrease during aging, metabolic disease, inflammatory diseases, during ischemia/reperfusion injury, and in other conditions in humans (Massudi et al., PLoS ONE. 2012, 7(7): e42357) and animals (Yang et al., Cell. 2007, 130(6):1095-107; Braidy et al. PLoS One. 2011, 26; 6(4):e19194; Peek et al. Science. 2013, 342(6158):1243417; Ghosh et al., J Neurosci. 2012, 32(17):5821-32), suggesting that modulation of cellular NAD+ level affects the speed and severity of the decline and deterioration of bodily functions. Therefore, an increase in cellular NAD+ concentration could be beneficial in the context of aging and age-related diseases.

The cellular NAD+ pool is controlled by a balance between the activity of NAD+-synthesizing and consuming enzymes. In mammals, NAD+ is synthesized from a variety of dietary sources, including one or more of its major precursors that include: tryptophan (Trp), nicotinic acid (NA), nicotinamide riboside (NR), nicotinamide mononucleotide (NMN), and nicotinamide (NAM). Based upon the bioavailability of its precursors, there are three pathways for the synthesis of NAD+ in cells: (i) from Trp by the de novo biosynthesis pathway or kynurenine pathway (ii) from NA in the Preiss-Handler pathway and (iii) from NAM, NR, and NMN in the salvage pathway (Verdin et al., Science. 2015, 350(6265):1208-13). Of these, the predominant NAD+ biosynthetic pathway involves the step of synthesis of nicotinamide mononucleotide (NMN) using nicotinamide and 5′-phosphoribosyl-pyrophosphate by the rate-limiting enzyme nicotinamide phosphoribosyl-transferase (NAMPT) that is critical to determination of longevity and responses to a variety of stresses (Fulco et al, Dev Cell. 2008, 14(5):661-73; Imai, Curr Pharm Des. 2009, 15(1):20-8; Revollo et al., J Biol Chem. 2004, 279(49):50754-63; Revollo et al., Cell Metab. 2007, November; 6(5):363-75; van der Veer et al., J Biol Chem. 2007, 282(15):10841-5; Yang et al., Cell. 2007, 130(6):1095-107). Thus, increasing the rate of NAMPT catalysis by a small molecule activator would be an effective strategy to boost NAD levels and thereby address a broad spectrum of disease states. These include cardiac diseases, chemotherapy induced tissue damage, renal diseases, metabolic diseases, muscular diseases, neurological diseases and injuries, diseases caused by impaired stem cell function, and DNA damage and primary mitochondrial disorders.

### SUMMARY

In one aspect, provided herein is a compound of Formula (II)

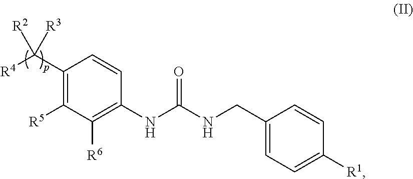

or a pharmaceutically acceptable salt thereof, wherein:
R¹ is halo or methoxy;
R⁶ is hydrogen or halo; and
p is 0 or 1, wherein
when p is 1,
R² is hydrogen or C₁-C₆ alkyl or is taken together with Z⁴ and the intervening atoms to form a 4-6 membered heterocycloalkyl or heterocycloalkenyl ring;
R³ is hydrogen or C₁-C₆ alkyl;
R⁴ is
   a) Z¹NR^{a}C(O)—,
   b) Z²C(O)NR^{b}—,
   c) Z³(CR^{c}R^{d})ₘNR^{e}—,
   d) Z⁴S(O)₂(CH₂)ₙ—,
   e) Z⁵OC(O)—,
   f) NR^{f}R^{g}C(O)—,
   g) 5- to 10-membered heteroaryl optionally substituted with one or more independently selected C₁-C₆ alkyl or C₃-C₆ cycloalkyl substituents,
   h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl optionally substituted with one or more independently selected oxo, 5- to 6-membered heteroaryl optionally substituted with one or more independently selected halo or —C₁-C₆ alkyl substituents, and C₃-C₆ cycloalkyl,
   i) Z⁶S(O)₂N(R^{s})—,
   j) Z⁷N(R^{t})S(O)₂—, or
   k) Z⁸—O—(CH₂)_{q}—; wherein
      R^{a} and R^{e} are each independently hydrogen or C₁-C₆ alkyl;
      R^{b} is hydrogen or C₁-C₆ alkyl or is taken together with R⁵ and the intervening atoms to form a 5- to 6-membered heterocycloalkyl or heterocycloalkenyl ring;
      R^{c} and R^{d} are each independently hydrogen or C₁-C₆ alkyl, or R^{c} and R^{d} together with the carbon to which they are attached form a C₃-C₆ cycloalkyl;
      R^{f} and R^{g} together with the nitrogen to which they are attached form a 3- to 10-membered heterocycloalkyl or heterocycloalkenyl optionally substituted with one or more independently selected substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl;
      each R^{h} is independently —C₁-C₆ alkyl, —O—C₁-C₆ alkyl, or C₆-C₁₂ aryl optionally substituted with one or more independently selected halo substituents;
   each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl;
   each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl;
   each R^{j}, R^{k}, R^{m}, Rⁿ, R^{o}, R^{p}, R^{g}, and R^{r} is independently hydrogen or C₁-C₆ alkyl;
   R^{s} is hydrogen or —C₁-C₆ alkyl;
   R^{t} is hydrogen or —C₁-C₆ alkyl;
   m is 0 or 1;
   n is 0, 1, or 2; and
   q is 0 or 1;
Z¹ and Z⁵ are each independently R^{z};
Z² and Z³ are each independently hydrogen or R^{z};
Z⁴ is hydrogen or R^{z} or is taken together with R² and the intervening atoms to form a 4-6 membered heterocycloalkyl or heterocycloalkenyl ring;
Z⁶ is selected from the group consisting of 5- to 6-membered heterocycloalkyl or heterocycloalkenyl, 5- to 6-membered heteroaryl, and C₁-C₆ alkyl;
Z⁷ is C₆-C₁₂ aryl;
Z⁸ is selected from the group consisting of 5- to 6-membered heteroaryl and C₃-C₆ cycloalkyl, and
R^{z} is selected from the group consisting of:
   a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy;
   b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
   c) C₁-C₆ alkoxy;
   d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R^{w} is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
   e) C₆-C₁₂ aryl; and
   f) 5- to 10-membered heteroaryl optionally substituted with one or more independently selected C₁-C₆ alkyl substituents; and
      R⁵ is hydrogen, halo, or is taken together with R^{b} and the intervening atoms form a 5- to 6-membered heterocycloalkyl or heterocycloalkenyl ring, provided that

(1) when R⁴ is Z¹NR^{a}C(O)—, Z¹ is other than methyl, unsubstituted cyclopropyl, —C(CH₃)₂CH₂OH, and —CH₂-thiofuran;

(2) R⁴ is other than 4-methylpiperazinyl, 4-phenylpiperazinyl, 4-pyridylpiperazinyl, 4-(furanylmethyl)piperazinyl,

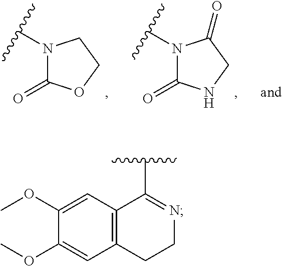 [SMILES: *N1CCOC1=O ; *N1C(=O)CNC1=O ; *C1=NCCc2cc(OC)c(OC)cc21]

and

(3) the compound of Formula (II) is not a compound of Table 1X; and

when p is 0, R⁴ is
l) 3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents,
m) 3- to 6-membered heterocycloalkyl or heterocycloalkenyl comprising exactly one annular heteroatom, which is an oxygen atom, wherein the 3- to 6-membered heterocycloalkyl or heterocycloalkenyl is optionally substituted with one or more independently selected oxo or —C₁-C₆ alkyl substituents,
n) 3- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents,
o) 5-membered heterocycloalkyl or heterocycloalkenyl comprising exactly two annular heteroatoms, one of which is a nitrogen atom and the other of which is an oxygen atom, wherein the 5-membered heterocycloalkyl or heterocycloalkenyl is optionally substituted with one or more independently selected oxo, C₁-C₆ alkyl, or —S(O)₂—(C₁-C₆ alkyl) substituents,
p) 6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, C₁-C₆ alkyl, or —S(O)₂—(C₁-C₆ alkyl) substituents,
q) 5-membered heteroaryl comprising exactly two annular heteroatoms, one of which is a nitrogen atom and the other of which is an oxygen atom, wherein the 5-membered heteroaryl is substituted with exactly one methyl substituent,
r) 5-membered heteroaryl comprising exactly two annular heteroatoms, both of which are nitrogen atoms, wherein the 5-membered heteroaryl is substituted with one or more methyl substituents,
s) 6-membered heteroaryl comprising one or two annular heteroatoms and optionally substituted with one or more methyl substituents, wherein the 6-membered heteroaryl is other than

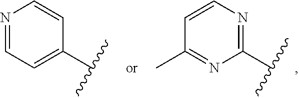

t) Z⁹—S(O)₂—,
u) Z¹⁰—S(O)₂—NH—,

### v) Z¹¹—C(O)—NH—,

w) Z¹²—CH₂—O—,

### x) Z¹³—O—,

y) Z¹⁴—C(H)(C₁-C₆ alkyl)-NH—C(O)—,
z)

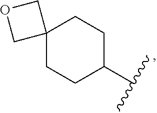 [SMILES: *C1CCC2(CC1)COC2]

or
aa)

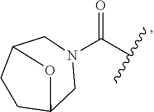

wherein

Z⁹ is selected from the group consisting of cyclopropyl, C₆-C₁₂ aryl, 3- to 10-membered heterocycloalkyl or hetercycloalkenyl optionally substituted with one or more independently selected R^{A} substituents, —NH(C₁-C₆ alkyl), —NH₂ substituted with one or more independently selected R^{B} substituents, and C₁-C₆ alkyl optionally substituted with one or more independently selected R^{C} substituents, provided that Z⁹ is other than

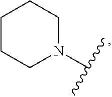 [SMILES: *N1CCCCC1]

unsubstituted methyl, or unsubstituted ethyl, wherein:
R^{A} is —C₁-C₆ alkyl or —CN; and
R^{B} is (i) —C₁-C₆ alkyl-(5- to 10-membered heteroaryl), or (ii) 5- to 10-membered heteroaryl optionally substituted with one or more independently selected C₆-C₁₂ aryl; and
R^{C} is 3- to 8-membered heterocycloalkyl or heterocycloalkenyl;

Z¹⁰ is C₁-C₆ alkyl substituted with one or more independently selected C₆-C₁₂ aryl substituents;

Z¹¹ is selected from the group consisting of C₃-C₁₀ cycloalkyl and C₁-C₆ alkyl substituted with one or more independently selected 3- to 10-membered heterocycloalkyl or hetercycloalkenyl substituents, provided that, when Z¹¹ is cyclopropyl, then R¹ is other than methoxy;

Z¹² is selected from the group consisting of C₆-C₁₂ aryl, 5- to 10-membered heteroaryl, 3- to 10-membered heterocycloalkyl or heterocycloalkenyl, C₁-C₆ alkyl substituted with one or more independently selected 3- to 10-membered heterocycloalkyl or hetercycloalkenyl substituents or 5- to 10-membered heteroaryl substituents, and —C(O)-(3- to 10-membered heterocycloalkyl or heterocycloalkenyl);

Z¹³ is 5- to 10-membered heteroaryl substituted with one or more independently selected —C(O)—NH(C₁-C₆ alkyl) substituents; and

Z¹⁴ is 5- to 10-membered heteroaryl optionally substituted with one or more independently selected C₁-C₆ alkyl substituents; and

R⁵ is hydrogen.

In another aspect, provided herein is a compound of Formula (I)

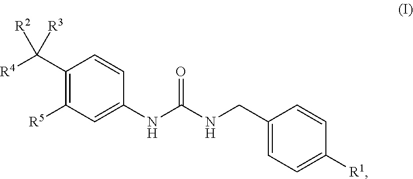

or a pharmaceutically acceptable salt thereof,
wherein:
R¹ is halo or methoxy;
R² is hydrogen or C₁-C₆ alkyl or is taken together with Z⁴ and the intervening atoms to form a 4-6 membered heterocycloalkyl or heterocycloalkenyl ring
R³ is hydrogen or C₁-C₆ alkyl;
R⁴ is
   a) Z¹NR^{a}C(O)—,
   b) Z²C(O)NR^{b}—,
   c) Z³(CR^{c}R^{d})ₘNR^{e}—,
   d) Z⁴S(O)₂(CH₂)ₙ—,
   e) Z⁵OC(O)—,
   f) NR^{f}R^{g}C(O)—,
   g) 5- to 10-membered heteroaryl optionally substituted with one or more independently selected C₁-C₆ alkyl substituents, or
   h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein
      R^{a} and R^{e} are each independently hydrogen or C₁-C₆ alkyl;
      R^{b} is hydrogen or C₁-C₆ alkyl or is taken together with R⁵ and the intervening atoms to form a 5- to 6-membered heterocycloalkyl or heterocycloalkenyl ring;
      R^{c} and R^{d} are each independently hydrogen or C₁-C₆ alkyl, or R^{c} and R^{d} together with the carbon to which they are attached form a C₃-C₆ cycloalkyl;
      R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl;
      each R^{h} is independently —C₁-C₆ alkyl, —O—C₁-C₆ alkyl, or C₆-C₁₂ aryl optionally substituted with one or more independently selected halo substituents;
   each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl;
   each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl;
   each R^{j}, R^{k}, R^{m}, Rⁿ, R^{o}, R^{p}, R^{g}, and R^{r} is independently hydrogen or C₁-C₆ alkyl;
   m is 0 or 1; and
   n is 0, 1, or 2;
R⁵ is hydrogen or is taken together with R^{b} and the intervening atoms form a 5- to 6-membered heterocycloalkyl or heterocycloalkenyl ring;
Z¹ and Z⁵ are each independently R^{z};
Z² and Z³ are each independently hydrogen or R^{z};
Z⁴ is hydrogen or R^{z} or is taken together with R² and the intervening atoms to form a 4-6 membered heterocycloalkyl or heterocycloalkenyl ring; and
R^{z} is selected from the group consisting of:
   a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy;
   b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
   c) C₁-C₆ alkoxy;
   d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R^{w} is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
   e) C₆-C₁₂ aryl; and
   f) 5- to 10-membered heteroaryl optionally substituted with one or more independently selected C₁-C₆ alkyl substituents,
   wherein (1) when R⁴ is Z¹NR^{a}C(O)—, Z¹ is other than methyl, unsubstituted cyclopropyl, —C(CH₃)₂CH₂OH, and —CH₂-thiofuran;

(2) R⁴ is other than 4-methylpiperazinyl, 4-phenylpiperazinyl, 4-pyridylpiperazinyl, 4-(furanylmethyl)piperazinyl,

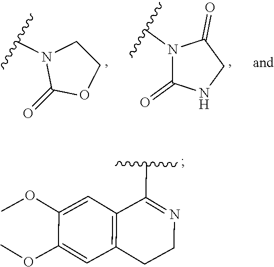 [SMILES: *N1CCOC1=O ; *N1C(=O)CNC1=O ; *C1=NCCc2cc(OC)c(OC)cc21]

and

(3) the compound of Formula (I) is not a compound of Table 1X.

In another aspect, provided herein is a compound of Formula (I-G)

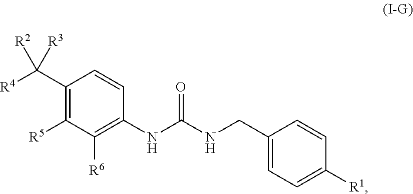

or a pharmaceutically acceptable salt thereof, wherein R¹, R², R³, R⁴, R⁵, and R⁶ are as defined for Formula (II) or any variation or embodiment thereof.

In another aspect, provided herein is a compound of Formula (I-A)

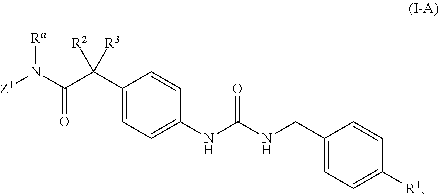

or a pharmaceutically acceptable salt thereof, wherein R¹, R², R³, R^{a}, and Z¹ are as defined for Formula (II) or any variation or embodiment thereof.

In another aspect, provided herein is a compound of Formula (I-B)

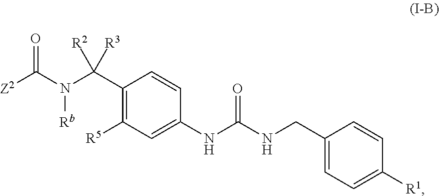

or a pharmaceutically acceptable salt thereof, wherein R¹, R², R³, R⁵, R^{b}, and Z² are as defined for Formula (II) or any variation or embodiment thereof.

In another aspect, provided herein is a compound of Formula (I-C)

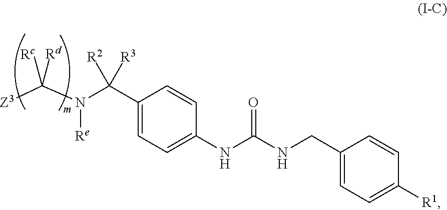

or a pharmaceutically acceptable salt thereof, wherein R¹, R², R³, R^{e}, R^{d}, R^{e}, m, and Z³ are as defined for Formula (II) or any variation or embodiment thereof.

In another aspect, provided herein is a compound of Formula (I-D)

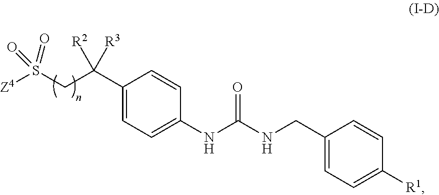

or a pharmaceutically acceptable salt thereof, wherein R¹, R², R³, n, and Z⁴ are as defined for Formula (II) or any variation or embodiment thereof.

In another aspect, provided herein is a compound of Formula (I-E)

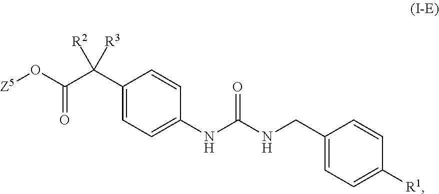

or a pharmaceutically acceptable salt thereof, wherein R¹, R², R³, and Z⁵ are as defined for Formula (II) or any variation or embodiment thereof.

In another aspect, provided herein is a compound of Formula (I-F)

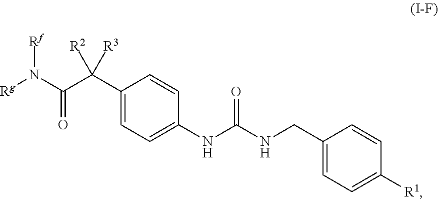

or a pharmaceutically acceptable salt thereof, wherein R¹, R², R³, R^{f}, and R^{g} are as defined for Formula (II) or any variation or embodiment thereof.

In another aspect, provided herein is a compound of Formula (II-A)

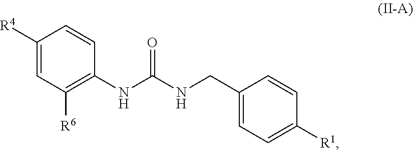

or a pharmaceutically acceptable salt thereof, wherein R¹, R⁴, and R⁶ are as defined for Formula (II) or any variation or embodiment thereof.

In a further aspect, provided herein are pharmaceutical compositions comprising at least one compound of Formula (II), (I-G), (I), (I-A), (I-B), (I-C), (I-D), (I-E), (I-F), or (II-A), such as a compound of Table 1, or a stereoisomer or tautomer thereof, or a pharmaceutically acceptable salt of any of the foregoing, optionally further comprising a pharmaceutically acceptable excipient.

In another aspect, provided herein is a method of treating a disease or condition mediated by NAMPT activity in a subject in need thereof, comprising administering to the subject an effective amount of at least one compound Formula (II), (I-G), (I), (I-A), (I-B), (I-C), (I-D), (I-E), (I-F), or (II-A), such as a compound of Table 1, or a pharmaceutically acceptable salt thereof, or a pharmaceutical composition comprising at least one compound of Formula (II), (I-G), (I), (I-A), (I-B), (I-C), (I-D), (I-E), (I-F), or (II-A). In some embodiments, the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder. In some embodiments, the disease or condition is selected from the group consisting of obesity, atherosclerosis, insulin resistance, type 2 diabetes, cardiovascular disease, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.

Additional embodiments, features, and advantages of the present disclosure will be apparent from the following detailed description and through practice of the present disclosure.

For the sake of brevity, the disclosures of publications cited in this specification, including patents, are herein incorporated by reference.

### DETAILED DESCRIPTION

### Definitions

As used in the present specification, the following words and phrases are generally intended to have the meanings as set forth below, except to the extent that the context in which they are used indicates otherwise.

Throughout this application, unless the context indicates otherwise, references to a compound of Formula (II) includes all subgroups of Formula (II) defined herein, such as Formula (I), (I-G), (I-A), (I-A1), (I-A2), (I-A3), (I-A4), (I-B), (I-B1), (I-B2), (I-B3), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1), including all substructures, subgenera, preferences, embodiments, examples and particular compounds defined and/or described herein. References to a compound of Formula (II) and subgroups thereof, such as Formula (I-G), (I) (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1), include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a compound of Formula (II) and subgroups thereof, such as Formula (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D32), (I-D33), (I-D4), (I-D5), (I-D6), (I-D7), (I-E), (I-F), (II-A), and (II-A1), include polymorphs, solvates, co-crystals, isomers, tautomers and/or oxides thereof. In some embodiments, references to a compound of Formula (II) and subgroups thereof, such as Formula (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1), include polymorphs, solvates, and/or co-crystals thereof. In some embodiments, references to a compound of Formula (II) and subgroups thereof, such as Formula (I-G), (I) (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1), include isomers, tautomers and/or oxides thereof. In some embodiments, references to a compound of Formula (II) and subgroups thereof, such as Formula (I-G), (I) (I-A), (I-A1), (I-A2), (I-A3), (I-A4), (I-B), (I-B1), (I-B2), (I-B3), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1), include solvates thereof. Similarly, the term “salts” includes solvates of salts of compounds.

“Alkyl” encompasses straight and branched carbon chains having the indicated number of carbon atoms, for example, from 1 to 20 carbon atoms, or 1 to 8 carbon atoms, or 1 to 6 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

“Cycloalkyl” indicates a non-aromatic, fully saturated carbocyclic ring having the indicated number of carbon atoms, for example, 3 to 10, or 3 to 8, or 3 to 6 ring carbon atoms. Cycloalkyl groups may be monocyclic or polycyclic (e.g., bicyclic, tricyclic). Examples of cycloalkyl groups include cyclopropyl, cyclobutyl, cyclopentyl, and cyclohexyl, as well as bridged, caged, and spirocyclic ring groups (e.g., norbornane, bicyclo[2.2.2]octane, spiro[3.3]heptane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Aryl” indicates an aromatic carbocyclic ring having the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl”, as defined herein, regardless of the point of attachment (e.g., both quinolin-5-yl and quinolin-2-yl are heteroaryl groups). In some instances, aryl is phenyl or naphthyl. In certain instances, aryl is phenyl. Additional examples of aryl groups comprising an aromatic carbon ring fused to a non-aromatic ring are described below.

“Heteroaryl” indicates an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups.

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

In some instances, both rings of a polycyclic heteroaryl group are aromatic. Examples include indole, isoindole, indazole, benzoimidazole, benzotriazole, benzofuran, benzoxazole, benzoisoxazole, benzoxadiazole, benzothiophene, benzothiazole, benzoisothiazole, benzothiadiazole, 1H-pyrrolo[2,3-b]pyridine, 1H-pyrazolo[3,4-b]pyridine, 3H-imidazo[4,5-b]pyridine, 3H-[1,2,3]triazolo[4,5-b]pyridine, 1H-pyrrolo[3,2-b]pyridine, 1H-pyrazolo[4,3-b]pyridine, 1H-imidazo[4,5-b]pyridine, 1H-[1,2,3]triazolo[4,5-b]pyridine, 1H-pyrrolo[2,3-c]pyridine, 1H-pyrazolo[3,4-c]pyridine, 3H-imidazo[4,5-c]pyridine, 3H-[1,2,3]triazolo[4,5-c]pyridine, 1H-pyrrolo[3,2-c]pyridine, 1H-pyrazolo[4,3-c]pyridine, 1H-imidazo[4,5-c]pyridine, 1H-[1,2,3]triazolo[4,5-c]pyridine, furo[2,3-b]pyridine, oxazolo[5,4-b]pyridine, isoxazolo[5,4-b]pyridine, [1,2,3]oxadiazolo[5,4-b]pyridine, furo[3,2-b]pyridine, oxazolo[4,5-b]pyridine, isoxazolo[4,5-b]pyridine, [1,2,3]oxadiazolo[4,5-b]pyridine, furo[2,3-c]pyridine, oxazolo[5,4-c]pyridine, isoxazolo[5,4-c]pyridine, [1,2,3]oxadiazolo[5,4-c]pyridine, furo[3,2-c]pyridine, oxazolo[4,5-c]pyridine, isoxazolo[4,5-c]pyridine, [1,2,3]oxadiazolo[4,5-c]pyridine, thieno[2,3-b]pyridine, thiazolo[5,4-b]pyridine, isothiazolo[5,4-b]pyridine, [1,2,3]thiadiazolo[5,4-b]pyridine, thieno[3,2-b]pyridine, thiazolo[4,5-b]pyridine, isothiazolo[4,5-b]pyridine, [1,2,3]thiadiazolo[4,5-b]pyridine, thieno[2,3-c]pyridine, thiazolo[5,4-c]pyridine, isothiazolo[5,4-c]pyridine, [1,2,3]thiadiazolo[5,4-c]pyridine, thieno[3,2-c]pyridine, thiazolo[4,5-c]pyridine, isothiazolo[4,5-c]pyridine, [1,2,3]thiadiazolo[4,5-c]pyridine, quinoline, isoquinoline, cinnoline, quinazoline, quinoxaline, phthalazine, naphthyridine (e.g., 1,8-naphthyridine, 1,7-naphthyridine, 1,6-naphthyridine, 1,5-naphthyridine, 2,7-naphthyridine, 2,6-naphthyridine), imidazo[1,2-a]pyridine, 1H-pyrazolo[3,4-d]thiazole, 1H-pyrazolo[4,3-d]thiazole and imidazo[2,1-b]thiazole.

In other instances, polycyclic heteroaryl groups may include a non-aromatic ring (e.g., cycloalkyl, cycloalkenyl, heterocycloalkyl, heterocycloalkenyl) fused to a heteroaryl ring, provided the polycyclic heteroaryl group is bound to the parent structure via an atom in the aromatic ring. For example, a 4,5,6,7-tetrahydrobenzo[d]thiazol-2-yl group (wherein the moiety is bound to the parent structure via an aromatic carbon atom) is considered a heteroaryl group, while 4,5,6,7-tetrahydrobenzo[d]thiazol-5-yl (wherein the moiety is bound to the parent structure via a non-aromatic carbon atom) is not considered a heteroaryl group. Examples of polycyclic heteroaryl groups consisting of a heteroaryl ring fused to a non-aromatic ring are described below.

“Heterocycloalkyl” indicates a non-aromatic, fully saturated ring having the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon. Heterocycloalkyl groups may be monocyclic or polycyclic (e.g., bicyclic, tricyclic). Examples of heterocycloalkyl groups include oxiranyl, aziridinyl, azetidinyl, pyrrolidinyl, imidazolidinyl, pyrazolidinyl, piperidinyl, piperazinyl, morpholinyl and thiomorpholinyl. Examples include thiomorpholine S-oxide and thiomorpholine S,S-dioxide. Examples of spirocyclic heterocycloalkyl groups include azaspiro[3.3]heptane, diazaspiro[3.3]heptane, diazaspiro[3.4]octane, and diazaspiro[3.5]nonane. In addition, one ring of a polycyclic heterocycloalkyl group may be aromatic (e.g., aryl or heteroaryl), provided the polycyclic heterocycloalkyl group is bound to the parent structure via a non-aromatic carbon or nitrogen atom. For example, a 1,2,3,4-tetrahydroquinolin-1-yl group (wherein the moiety is bound to the parent structure via a non-aromatic nitrogen atom) is considered a heterocycloalkyl group, while 1,2,3,4-tetrahydroquinolin-8-yl group (wherein the moiety is bound to the parent structure via an aromatic carbon atom) is not considered a heterocycloalkyl group. Examples of polycyclic heterocycloalkyl groups consisting of a heterocycloalkyl group fused to an aromatic ring are described below.

“Heterocycloalkenyl” indicates a non-aromatic ring having the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon, and at least one double bond derived by the removal of one molecule of hydrogen from adjacent carbon atoms, adjacent nitrogen atoms, or adjacent carbon and nitrogen atoms of the corresponding heterocycloalkyl. Heterocycloalkenyl groups may be monocyclic or polycyclic (e.g., bicyclic, tricyclic). Examples of heterocycloalkenyl groups include dihydrofuranyl (e.g., 2,3-dihydrofuranyl, 2,5-dihydrofuranyl), dihydrothiophenyl (e.g., 2,3-dihydrothiophenyl, 2,5-dihydrothiophenyl), dihydropyrrolyl (e.g., 2,3-dihydro-1H-pyrrolyl, 2,5-dihydro-1H-pyrrolyl), dihydroimidazolyl (e.g., 2,3-dihydro-1H-imidazolyl, 4,5-dihydro-1H-imidazolyl), pyranyl, dihydropyranyl (e.g., 3,4-dihydro-2H-pyranyl, 3,6-dihydro-2H-pyranyl), tetrahydropyridinyl (e.g., 1,2,3,4-tetrahydropyridinyl, 1,2,3,6-tetrahydropyridinyl) and dihydropyridine (e.g., 1,2-dihydropyridine, 1,4-dihydropyridine). In addition, one ring of a polycyclic heterocycloalkenyl group may be aromatic (e.g., aryl or heteroaryl), provided the polycyclic heterocycloalkenyl group is bound to the parent structure via a non-aromatic carbon or nitrogen atom. For example, a 1,2-dihydroquinolin-1-yl group (wherein the moiety is bound to the parent structure via a non-aromatic nitrogen atom) is considered a heterocycloalkenyl group, while 1,2-dihydroquinolin-8-yl group (wherein the moiety is bound to the parent structure via an aromatic carbon atom) is not considered a heterocycloalkenyl group. Examples of polycyclic heterocycloalkenyl groups consisting of a heterocycloalkenyl group fused to an aromatic ring are described below.

Examples of polycyclic rings consisting of an aromatic ring (e.g., aryl or heteroaryl) fused to a non-aromatic ring (e.g., cycloalkyl, cycloalkenyl, heterocycloalkyl, heterocycloalkenyl) include indenyl, 2,3-dihydro-1H-indenyl, 1,2,3,4-tetrahydronaphthalenyl, benzo[1,3]dioxolyl, tetrahydroquinolinyl, 2,3-dihydrobenzo[1,4]dioxinyl, indolinyl, isoindolinyl, 2,3-dihydro-1H-indazolyl, 2,3-dihydro-1H-benzo[d]imidazolyl, 2,3-dihydrobenzofuranyl, 1,3-dihydroisobenzofuranyl, 1,3-dihydrobenzo[c]isoxazolyl, 2,3-dihydrobenzo[d]isoxazolyl, 2,3-dihydrobenzo[d]oxazolyl, 2,3-dihydrobenzo[b]thiophenyl, 1,3-dihydrobenzo[c]thiophenyl, 1,3-dihydrobenzo[c]isothiazolyl, 2,3-dihydrobenzo[d]isothiazolyl, 2,3-dihydrobenzo[d]thiazolyl, 5,6-dihydro-4H-cyclopenta[d]thiazolyl, 4,5,6,7-tetrahydrobenzo[d]thiazolyl, 5,6-dihydro-4H-pyrrolo[3,4-d]thiazolyl, 4,5,6,7-tetrahydrothiazolo[5,4-c]pyridinyl, indolin-2-one, indolin-3-one, isoindolin-1-one, 1,2-dihydroindazol-3-one, 1H-benzo[d]imidazol-2(3H)-one, benzofuran-2(3H)-one, benzofuran-3(2H)-one, isobenzofuran-1(3H)-one, benzo[c]isoxazol-3(1H)-one, benzo[d]isoxazol-3(2H)-one, benzo[d]oxazol-2(3H)-one, benzo[b]thiophen-2(3H)-one, benzo[b]thiophen-3(2H)-one, benzo[c]thiophen-1(3H)-one, benzo[c]isothiazol-3(1H)-one, benzo[d]isothiazol-3(2H)-one, benzo[d]thiazol-2(3H)-one, 4,5-dihydropyrrolo[3,4-d]thiazol-6-one, 1,2-dihydropyrazolo[3,4-d]thiazol-3-one, quinolin-4(3H)-one, quinazolin-4(3H)-one, quinazoline-2,4(1H,3H)-dione, quinoxalin-2(1H)-one, quinoxaline-2,3(1H,4H)-dione, cinnolin-4(3H)-one, pyridin-2(1H)-one, pyrimidin-2(1H)-one, pyrimidin-4(3H)-one, pyridazin-3(2H)-one, 1H-pyrrolo[3,2-b]pyridin-2(3H)-one, 1H-pyrrolo[3,2-c]pyridin-2(3H)-one, 1H-pyrrolo[2,3-c]pyridin-2(3H)-one, 1H-pyrrolo[2,3-b]pyridin-2(3H)-one, 1,2-dihydropyrazolo[3,4-d]thiazol-3-one and 4,5-dihydropyrrolo[3,4-d]thiazol-6-one. As discussed herein, whether each ring is considered an aryl, heteroaryl, cycloalkyl, cycloalkenyl, heterocycloalkyl or heterocycloalkenyl group is determined by the atom through which the moiety is bound to the parent structure.

“Halogen” or “halo” refers to fluorine, chlorine, bromine or iodine.

Unless otherwise indicated, compounds disclosed and/or described herein include all possible enantiomers, diastereomers, meso isomers and other stereoisomeric forms, including racemic mixtures, optically pure forms and intermediate mixtures thereof. Enantiomers, diastereomers, meso isomers and other stereoisomeric forms can be prepared using chiral synthons or chiral reagents, or resolved using conventional techniques. Unless specified otherwise, when the compounds disclosed and/or described herein contain olefinic double bonds or other centers of geometric asymmetry, it is intended that the compounds include both E and Z isomers. When the compounds described herein contain moieties capable of tautomerization, and unless specified otherwise, it is intended that the compounds include all possible tautomers.

“Protecting group” has the meaning conventionally associated with it in organic synthesis, i.e., a group that selectively blocks one or more reactive sites in a multifunctional compound such that a chemical reaction can be carried out selectively on another unprotected reactive site, and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a “hydroxy protected form” contains at least one hydroxy group protected with a hydroxy protecting group. Likewise, amines and other reactive groups may similarly be protected.

The term “pharmaceutically acceptable salt” refers to a salt of any of the compounds herein which are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

If the compound described herein is obtained as an acid addition salt, the free base can be obtained by basifying a solution of the acid salt. Conversely, if the compound is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds (see, e.g., Berge et al., Pharmaceutical Salts, J. Pharmaceutical Sciences, January 1977, 66(1), 1-19). Those skilled in the art will recognize various synthetic methodologies that may be used to prepare pharmaceutically acceptable addition salts.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

The term “substituted” means that the specified group or moiety bears one or more substituents including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocycloalkyl, heterocycloalkenyl, aralkyl, aminosulfonyl, sulfonylamino, sulfonyl, oxo, carbonylalkylenealkoxy and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The compounds disclosed and/or described herein can be enriched isotopic forms, e.g., enriched in the content of ²H, ³H, ¹¹C, ¹³C and/or ¹⁴C. In one embodiment, the compound contains at least one deuterium atom. Such deuterated forms can be made, for example, by the procedure described in U.S. Pat. Nos. 5,846,514 and 6,334,997. Such deuterated compounds may improve the efficacy and increase the duration of action of compounds disclosed and/or described herein. Deuterium substituted compounds can be synthesized using various methods, such as those described in: Dean, D., Recent Advances in the Synthesis and Applications of Radiolabeled Compounds for Drug Discovery and Development, Curr. Pharm. Des., 2000; 6(10); Kabalka, G. et al., The Synthesis of Radiolabeled Compounds via Organometallic Intermediates, Tetrahedron, 1989, 45(21), 6601-21; and Evans, E., Synthesis of radiolabeled compounds, J. Radioanal. Chem., 1981, 64(1-2), 9-32.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The terms “patient,” “individual,” and “subject” refer to an animal, such as a mammal, bird, or fish. In some embodiments, the patient or subject is a mammal. Mammals include, for example, mice, rats, dogs, cats, pigs, sheep, horses, cows and humans. In some embodiments, the patient or subject is a human, for example a human that has been or will be the object of treatment, observation or experiment. The compounds, compositions and methods described herein can be useful in both human therapy and veterinary applications.

As used herein, the term “therapeutic” refers to the ability to modulate nicotinamide phosphoribosyltransferase (NAMPT). As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the chemical entity with the a target or due to the interaction of the chemical entity with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

The term “therapeutically effective amount” or “effective amount” refers to that amount of a compound disclosed and/or described herein that is sufficient to affect treatment, as defined herein, when administered to a patient in need of such treatment. A therapeutically effective amount of a compound may be an amount sufficient to treat a disease responsive to modulation of nicotinamide phosphoribosyltransferase (NAMPT). The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

“Treatment” (and related terms, such as “treat”, “treated”, “treating”) includes one or more of: preventing a disease or disorder (i.e., causing the clinical symptoms of the disease or disorder not to develop); inhibiting a disease or disorder; slowing or arresting the development of clinical symptoms of a disease or disorder; and/or relieving a disease or disorder (i.e., causing relief from or regression of clinical symptoms). The term encompasses situations where the disease or disorder is already being experienced by a patient, as well as situations where the disease or disorder is not currently being experienced but is expected to arise. The term covers both complete and partial reduction or prevention of the condition or disorder, and complete or partial reduction of clinical symptoms of a disease or disorder. Thus, compounds described and/or disclosed herein may prevent an existing disease or disorder from worsening, assist in the management of the disease or disorder, or reduce or eliminate the disease or disorder. When used in a prophylactic manner, the compounds disclosed and/or described herein may prevent a disease or disorder from developing or lessen the extent of a disease or disorder that may develop.

### Compounds

Compounds and salts thereof (such as pharmaceutically acceptable salts) are detailed herein, including in the Brief Summary and in the appended claims. Also provided are the use of all of the compounds described herein, including any and all stereoisomers, including geometric isomers (cis/trans), E/Z isomers, enantiomers, diastereomers, and mixtures thereof in any ratio including racemic mixtures, salts and solvates of the compounds described herein, as well as methods of making such compounds. Any compound described herein may also be referred to as a drug.

In one aspect, provided are compounds of Formula (II)

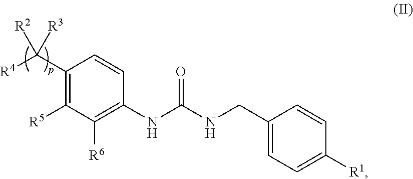

or a pharmaceutically acceptable salt thereof, wherein:
R¹ is halo or methoxy;
R⁶ is hydrogen or halo; and
p is 0 or 1, wherein
when p is 1,
R² is hydrogen or C₁-C₆ alkyl or is taken together with Z⁴ and the intervening atoms to form a 4-6 membered heterocycloalkyl or heterocycloalkenyl ring;
R³ is hydrogen or C₁-C₆ alkyl;
R⁴ is
   a) Z¹NR^{a}C(O)—,
   b) Z²C(O)NR^{b}—,
   c) Z³(CR^{c}R^{d})ₘNR^{e}—,
   d) Z⁴S(O)₂(CH₂)ₙ—,
   e) Z⁵OC(O)—,
   f) NR^{f}R^{g}C(O)—,
   g) 5- to 10-membered heteroaryl optionally substituted with one or more independently selected C₁-C₆ alkyl or C₃-C₆ cycloalkyl substituents,
   h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl optionally substituted with one or more independently selected oxo, 5- to 6-membered heteroaryl optionally substituted with one or more independently selected halo or —C₁-C₆ alkyl substituents, and C₃-C₆ cycloalkyl,
   i) Z⁶S(O)₂N(R^{s})—,
   j) Z⁷N(R^{t})S(O)₂—, or
   k) Z⁸—O—(CH₂)_{q}—; wherein
      R^{a} and R^{e} are each independently hydrogen or C₁-C₆ alkyl;
      R^{b} is hydrogen or C₁-C₆ alkyl or is taken together with R^{s} and the intervening atoms to form a 5- to 6-membered heterocycloalkyl or heterocycloalkenyl ring;
      R^{c} and R^{d} are each independently hydrogen or C₁-C₆ alkyl, or R^{c} and R^{d} together with the carbon to which they are attached form a C₃-C₆ cycloalkyl;
      R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl;
      each R^{h} is independently —C₁-C₆ alkyl, —O—C₁-C₆ alkyl, or C₆-C₁₂ aryl optionally substituted with one or more independently selected halo substituents;
   each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl;
   each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl;
   each R^{j}, R^{k}, R^{m}, Rⁿ, R^{o}, R^{p}, R^{g}, and R^{r} is independently hydrogen or C₁-C₆ alkyl;
   R^{s} is hydrogen or —C₁-C₆ alkyl;
   R^{t} is hydrogen or —C₁-C₆ alkyl;
   m is 0 or 1;
   n is 0, 1, or 2; and
   q is 0 or 1;
Z¹ and Z⁵ are each independently R^{z};
Z² and Z³ are each independently hydrogen or R^{z};
Z⁴ is hydrogen or R^{z} or is taken together with R² and the intervening atoms to form a 4-6 membered heterocycloalkyl or heterocycloalkenyl ring;
Z⁶ is selected from the group consisting of 5- to 6-membered heterocycloalkyl or heterocycloalkenyl, 5- to 6-membered heteroaryl, and C₁-C₆ alkyl;
Z⁷ is C₆-C₁₂ aryl;
Z⁸ is selected from the group consisting of 5- to 6-membered heteroaryl and C₃-C₆ cycloalkyl, and
R^{z} is selected from the group consisting of:
   a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy;
   b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
   c) C₁-C₆ alkoxy;
   d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R^{w} is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
   e) C₆-C₁₂ aryl; and
   f) 5- to 10-membered heteroaryl optionally substituted with one or more independently selected C₁-C₆ alkyl substituents; and
      R⁵ is hydrogen, halo, or is taken together with R^{b} and the intervening atoms form a 5- to 6-membered heterocycloalkyl or heterocycloalkenyl ring, provided that

(1) when R⁴ is Z¹NR^{a}C(O)—, Z¹ is other than methyl, unsubstituted cyclopropyl, —C(CH₃)₂CH₂OH, and —CH₂-thiofuran;

(2) R⁴ is other than 4-methylpiperazinyl, 4-phenylpiperazinyl, 4-pyridylpiperazinyl, 4-(furanylmethyl)piperazinyl,

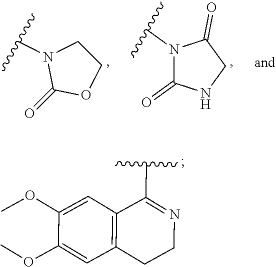 [SMILES: *N1CCOC1=O ; *N1C(=O)CNC1=O ; *C1=NCCc2cc(OC)c(OC)cc21]

and

(3) the compound of Formula (II) is not a compound of Table 1X; and when p is 0, R⁴ is

l) 3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents,
m) 3- to 6-membered heterocycloalkyl or heterocycloalkenyl comprising exactly one annular heteroatom, which is an oxygen atom, wherein the 3- to 6-membered heterocycloalkyl or heterocycloalkenyl is optionally substituted with one or more independently selected oxo or —C₁-C₆ alkyl substituents,
n) 3- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents,
o) 5-membered heterocycloalkyl or heterocycloalkenyl comprising exactly two annular heteroatoms, one of which is a nitrogen atom and the other of which is an oxygen atom, wherein the 5-membered heterocycloalkyl or heterocycloalkenyl is optionally substituted with one or more independently selected oxo, C₁-C₆ alkyl, or —S(O)₂—(C₁-C₆ alkyl) substituents,
p) 6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, C₁-C₆ alkyl, or —S(O)₂—(C₁-C₆ alkyl) substituents,
q) 5-membered heteroaryl comprising exactly two annular heteroatoms, one of which is a nitrogen atom and the other of which is an oxygen atom, wherein the 5-membered heteroaryl is substituted with exactly one methyl substituent,
r) 5-membered heteroaryl comprising exactly two annular heteroatoms, both of which are nitrogen atoms, wherein the 5-membered heteroaryl is substituted with one or more methyl substituents,
s) 6-membered heteroaryl comprising one or two annular heteroatoms and optionally substituted with one or more methyl substituents, wherein the 6-membered heteroaryl is other than

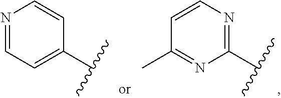

t) Z⁹—S(O)₂—,
u) Z¹⁰—S(O)₂—NH—,

### v) Z¹¹—C(O)—NH—,

w) Z¹²—CH₂—O—,

### x) Z¹³—O—,

y) Z¹⁴—C(H)(C₁-C₆ alkyl)-NH—C(O)—,
z)

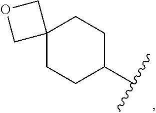 [SMILES: *C1CCC2(CC1)COC2]

or
aa)

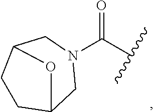

wherein

Z⁹ is selected from the group consisting of cyclopropyl, C₆-C₁₂ aryl, 3- to 10-membered heterocycloalkyl or hetercycloalkenyl optionally substituted with one or more independently selected R^{A} substituents, —NH(C₁-C₆ alkyl), —NH₂ substituted with one or more independently selected R^{B} substituents, and C₁-C₆ alkyl optionally substituted with one or more independently selected R^{C} substituents, provided that Z⁹ is other than

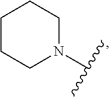 [SMILES: *N1CCCCC1]

unsubstituted methyl, or unsubstituted ethyl, wherein:
R^{A} is —C₁-C₆ alkyl or —CN; and
R^{B} is (i) —C₁-C₆ alkyl-(5- to 10-membered heteroaryl), or (ii) 5- to 10-membered heteroaryl optionally substituted with one or more independently selected C₆-C₁₂ aryl; and
R^{C} is 3- to 8-membered heterocycloalkyl or heterocycloalkenyl;

Z¹⁰ is C₁-C₆ alkyl substituted with one or more independently selected C₆-C₁₂ aryl substituents;

Z¹¹ is selected from the group consisting of C₃-C₁₀ cycloalkyl and C₁-C₆ alkyl substituted with one or more independently selected 3- to 10-membered heterocycloalkyl or hetercycloalkenyl substituents, provided that, when Z¹¹ is cyclopropyl, then R¹ is other than methoxy;

Z¹² is selected from the group consisting of C₆-C₁₂ aryl, 5- to 10-membered heteroaryl, 3- to 10-membered heterocycloalkyl or heterocycloalkenyl, C₁-C₆ alkyl substituted with one or more independently selected 3- to 10-membered heterocycloalkyl or hetercycloalkenyl substituents or 5- to 10-membered heteroaryl substituents, and —C(O)-(3- to 10-membered heterocycloalkyl or heterocycloalkenyl);

Z¹³ is 5- to 10-membered heteroaryl substituted with one or more independently selected —C(O)—NH(C₁-C₆ alkyl) substituents; and

Z¹⁴ is 5- to 10-membered heteroaryl optionally substituted with one or more independently selected C₁-C₆ alkyl substituents; and

R⁵ is hydrogen.

In one aspect, provided are compounds of Formula (I-G)

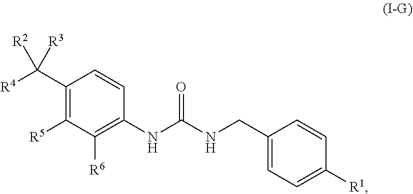

or a pharmaceutically acceptable salt thereof, wherein:
R¹ is halo or methoxy;
R² is hydrogen or C₁-C₆ alkyl or is taken together with Z⁴ and the intervening atoms to form a 4-6 membered heterocycloalkyl or heterocycloalkenyl ring;
R³ is hydrogen or C₁-C₆ alkyl;
R⁴ is
   a) Z¹NR^{a}C(O)—,
   b) Z²C(O)NR^{b}—,
   c) Z³(CR^{c}R^{d})ₘNR^{e}—,
   d) Z⁴S(O)₂(CH₂)ₙ—,
   e) Z⁵OC(O)—,
   f) NR^{f}R^{g}C(O)—,
   g) 5- to 10-membered heteroaryl optionally substituted with one or more independently selected C₁-C₆ alkyl or C₃-C₆ cycloalkyl substituents,
   h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl optionally substituted with one or more independently selected oxo, 5- to 6-membered heteroaryl optionally substituted with one or more independently selected halo or —C₁-C₆ alkyl substituents, and C₃-C₆ cycloalkyl,
   i) Z⁶S(O)₂N(R^{s})—,
   j) Z⁷N(R^{t})S(O)₂—, or
   k) Z⁸—O—(CH₂)_{q}—; wherein
      R^{a} and R^{e} are each independently hydrogen or C₁-C₆ alkyl;
      R^{b} is hydrogen or C₁-C₆ alkyl or is taken together with R⁵ and the intervening atoms to form a 5- to 6-membered heterocycloalkyl or heterocycloalkenyl ring;
      R^{c} and R^{d} are each independently hydrogen or C₁-C₆ alkyl, or R^{c} and R^{d} together with the carbon to which they are attached form a C₃-C₆ cycloalkyl;
      R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl;
      each R^{h} is independently —C₁-C₆ alkyl, —O—C₁-C₆ alkyl, or C₆-C₁₂ aryl optionally substituted with one or more independently selected halo substituents;
   each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl;
   each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl; each R^{j}, R^{k}, R^{m}, Rⁿ, R^{o}, R^{p}, R^{g}, and R^{r} is independently hydrogen or C₁-C₆ alkyl;
   R^{s} is hydrogen or —C₁-C₆ alkyl;
   R^{t} is hydrogen or —C₁-C₆ alkyl;
   m is 0 or 1;
   n is 0, 1, or 2;
   q is 0 or 1;
Z² and Z³ are each independently hydrogen or R^{z};
Z⁴ is hydrogen or R^{z} or is taken together with R² and the intervening atoms to form a 4-6 membered heterocycloalkyl or heterocycloalkenyl ring;
Z⁶ is selected from the group consisting of 5- to 6-membered heterocycloalkyl or heterocycloalkenyl, 5- to 6-membered heteroaryl, and C₁-C₆ alkyl;
Z⁷ is C₆-C₁₂ aryl;
Z⁸ is selected from the group consisting of 5- to 6-membered heteroaryl and C₃-C₆ cycloalkyl, and
R^{z} is selected from the group consisting of:
   a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy;
   b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
   c) C₁-C₆ alkoxy;
   d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R^{w} is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
   e) C₆-C₁₂ aryl; and
   f) 5- to 10-membered heteroaryl optionally substituted with one or more independently selected C₁-C₆ alkyl substituents;
      R⁵ is hydrogen, halo, or is taken together with R^{b} and the intervening atoms form a 5- to 6-membered heterocycloalkyl or heterocycloalkenyl ring; and
      R⁶ is hydrogen or halo, Z¹ and Z⁵ are each independently R^{z}, provided that

(1) when R⁴ is Z¹NR^{a}C(O)—, Z¹ is other than methyl, unsubstituted cyclopropyl, —C(CH₃)₂CH₂OH, and —CH₂-thiofuran;

(2) R⁴ is other than 4-methylpiperazinyl, 4-phenylpiperazinyl, 4-pyridylpiperazinyl, 4-(furanylmethyl)piperazinyl,

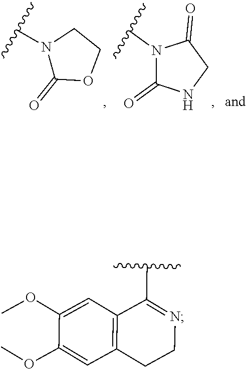

and

(3) the compound of Formula (I-G) is not a compound of Table 1X.

In one aspect, provided are compounds of Formula (I)

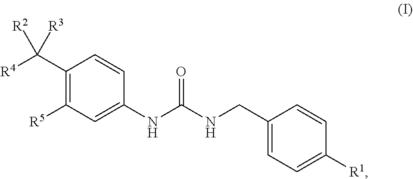

or a pharmaceutically acceptable salt thereof,
wherein:
R¹ is halo or methoxy;
R² is hydrogen or C₁-C₆ alkyl or is taken together with Z⁴ and the intervening atoms to form a 4-6 membered heterocycloalkyl or heterocycloalkenyl ring;
R³ is hydrogen or C₁-C₆ alkyl;
R⁴ is
   a) Z¹NR^{a}C(O)—,
   b) Z²C(O)NR^{b}—,
   c) Z³(CR^{c}R^{d})ₘNR^{e}—,
   d) Z⁴S(O)₂(CH₂)ₙ—,
   e) Z⁵OC(O)—,
   f) NR^{f}R^{g}C(O)—,
   g) 5- to 10-membered heteroaryl optionally substituted with one or more independently selected C₁-C₆ alkyl substituents, or
   h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein
      R^{a} and R^{e} are each independently hydrogen or C₁-C₆ alkyl;
      R^{b} is hydrogen or C₁-C₆ alkyl or is taken together with R⁵ and the intervening atoms to form a 5- to 6-membered heterocycloalkyl or heterocycloalkenyl ring;
      R^{c} and R^{d} are each independently hydrogen or C₁-C₆ alkyl, or R^{c} and R^{d} together with the carbon to which they are attached form a C₃-C₆ cycloalkyl;
      R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl;
      each R^{h} is independently —C₁-C₆ alkyl, —O—C₁-C₆ alkyl, or C₆-C₁₂ aryl optionally substituted with one or more independently selected halo substituents;
   each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl;
   each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl;
   each R^{j}, R^{k}, R^{m}, Rⁿ, R^{o}, R^{p}, R^{g}, and R^{r} is independently hydrogen or C₁-C₆ alkyl;
   m is 0 or 1; and
   n is 0, 1, or 2;
R⁵ is hydrogen or is taken together with R^{b} and the intervening atoms form a 5- to 6-membered heterocycloalkyl or heterocycloalkenyl ring;
Z¹ and Z⁵ are each independently R^{z};
Z² and Z³ are each independently hydrogen or R^{z};
Z⁴ is hydrogen or R^{z} or is taken together with R² and the intervening atoms to form a 4-6 membered heterocycloalkyl or heterocycloalkenyl ring; and
R^{z} is selected from the group consisting of:
   a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy;
   b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
   c) C₁-C₆ alkoxy;
   d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R^{w} is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
   e) C₆-C₁₂ aryl; and
   f) 5- to 10-membered heteroaryl optionally substituted with one or more independently selected C₁-C₆ alkyl substituents.

In some embodiments of Formula (II), Formula (I-G), or Formula (I), (1) when R⁴ is Z¹NR^{a}C(O)—, Z¹ is other than methyl, unsubstituted cyclopropyl, —C(CH₃)₂CH₂OH, and —CH₂-thiofuran; (2) R⁴ is other than 4-methylpiperazinyl, 4-phenylpiperazinyl, 4-pyridylpiperazinyl, 4-(furanylmethyl)piperazinyl,

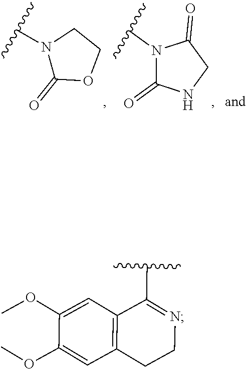

and (3) the compound of Formula (II), Formula (I-G), or Formula (I) is not a compound of Table 1X.

TABLE 1X

| Compound No. | Structure |
|---|---|
| X1 | 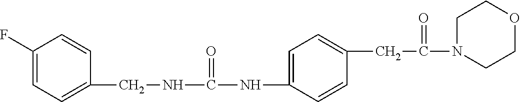 [SMILES: O=C(NCc1ccc(F)cc1)Nc1ccc(CC(=O)N2CCOCC2)cc1] |
| X2 | 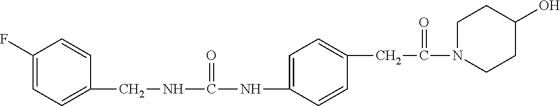 [SMILES: O=C(NCc1ccc(F)cc1)Nc1ccc(CC(=O)N2CCC(O)CC2)cc1] |
| X3 | 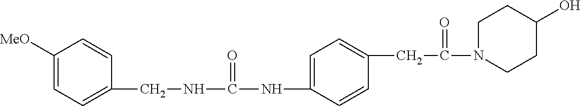 [SMILES: COc1ccc(CNC(=O)Nc2ccc(CC(=O)N3CCC(O)CC3)cc2)cc1] |
| X4 | 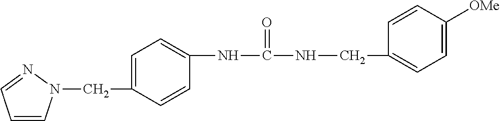 [SMILES: COc1ccc(CNC(=O)Nc2ccc(Cn3cccn3)cc2)cc1] |
| X5 | 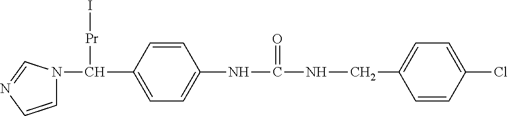 |
| X6 | 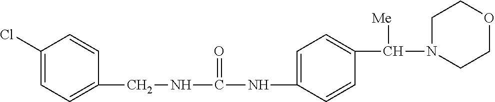 [SMILES: CC(c1ccc(NC(=O)NCc2ccc(Cl)cc2)cc1)N1CCOCC1] |
| X7 | 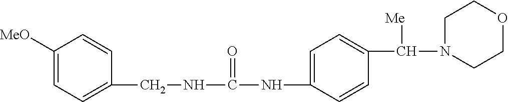 [SMILES: COc1ccc(CNC(=O)Nc2ccc(C(C)N3CCOCC3)cc2)cc1] |
| X8 | 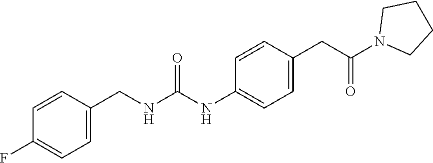 [SMILES: O=C(NCc1ccc(F)cc1)Nc1ccc(CC(=O)N2CCCC2)cc1] |
| X9 | 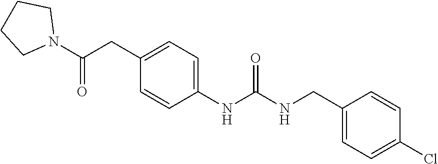 [SMILES: O=C(NCc1ccc(Cl)cc1)Nc1ccc(CC(=O)N2CCCC2)cc1] |
| X10 | 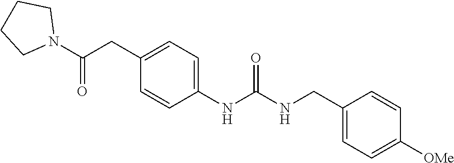 [SMILES: COc1ccc(CNC(=O)Nc2ccc(CC(=O)N3CCCC3)cc2)cc1] |
| X11 | 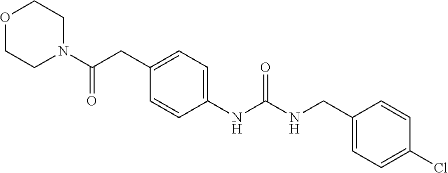 [SMILES: O=C(NCc1ccc(Cl)cc1)Nc1ccc(CC(=O)N2CCOCC2)cc1] |
| X12 | 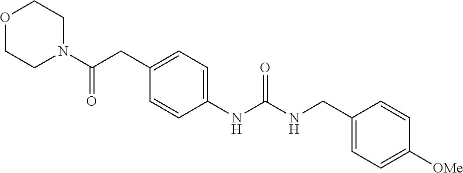 [SMILES: COc1ccc(CNC(=O)Nc2ccc(CC(=O)N3CCOCC3)cc2)cc1] |
| X13 | 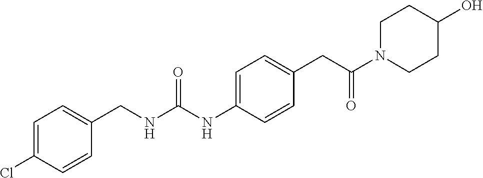 [SMILES: O=C(NCc1ccc(Cl)cc1)Nc1ccc(CC(=O)N2CCC(O)CC2)cc1] |
| X14 | 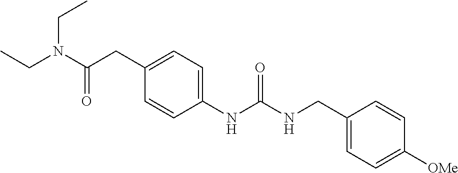 [SMILES: CCN(CC)C(=O)Cc1ccc(NC(=O)NCc2ccc(OC)cc2)cc1] |
| X15 | 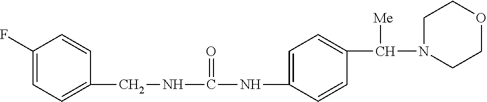 [SMILES: CC(c1ccc(NC(=O)NCc2ccc(F)cc2)cc1)N1CCOCC1] |
| X16 | 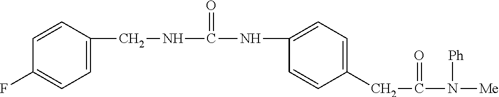 [SMILES: CN(C(=O)Cc1ccc(NC(=O)NCc2ccc(F)cc2)cc1)c1ccccc1] |
| X17 | 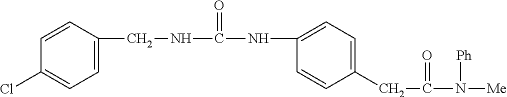 [SMILES: CN(C(=O)Cc1ccc(NC(=O)NCc2ccc(Cl)cc2)cc1)c1ccccc1] |
| X18 | 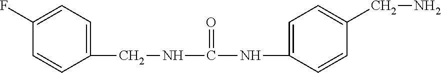 [SMILES: NCc1ccc(NC(=O)NCc2ccc(F)cc2)cc1] |
| X19 | 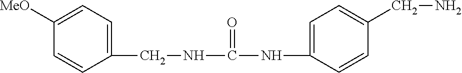 [SMILES: COc1ccc(CNC(=O)Nc2ccc(CN)cc2)cc1] |
| X20 | 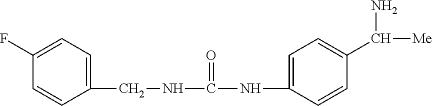 [SMILES: CC(N)c1ccc(NC(=O)NCc2ccc(F)cc2)cc1] |
| X21 | 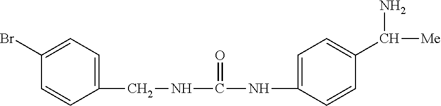 [SMILES: CC(N)c1ccc(NC(=O)NCc2ccc(Br)cc2)cc1] |
| X22 | 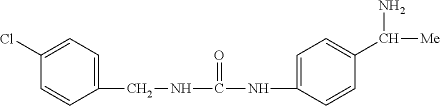 [SMILES: CC(N)c1ccc(NC(=O)NCc2ccc(Cl)cc2)cc1] |
| X23 | 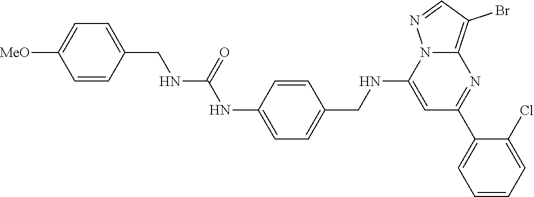 [SMILES: COc1ccc(CNC(=O)Nc2ccc(CNc3cc(-c4ccccc4Cl)nc4c(Br)cnn34)cc2)cc1] |

In some embodiments of Formula (II), Formula (I-G), or Formula (I), R¹ is halo. For example, in some embodiments, R¹ is fluoro. In some embodiments, R¹ is chloro. In some embodiments, R¹ is bromo. In other embodiments, R¹ is iodo.

In some embodiments of Formula (II), Formula (I-G), or Formula (I), R¹ is methoxy.

In some embodiments of Formula (II), Formula (I-G), or Formula (I), R² is hydrogen. In some embodiments, R² is C₁-C₆ alkyl. For example, in some embodiments, R² is methyl, ethyl, propyl, isopropyl, n-butyl, sec-butyl, or tert-butyl.

In some embodiments of Formula (II), Formula (I-G), or Formula (I), R³ is hydrogen. In some embodiments, R³ is C₁-C₆ alkyl. For example, in some embodiments, R³ is methyl, ethyl, propyl, isopropyl, n-butyl, sec-butyl, or tert-butyl.

In some embodiments of Formula (II), Formula (I-G), or Formula (I), R^{s} is hydrogen. In some embodiments, R^{b}, if present, is taken together with R^{s} and the intervening atoms to form a 5- to 6-membered heterocycloalkyl or heterocycloalkenyl ring. In some embodiments, R⁵ is halo. In some embodiments, R⁵ is fluoro. In some embodiments, R⁵ is chloro. In some embodiments, R⁵ is bromo. In some embodiments, R⁵ is iodo.

In some embodiments of Formula (II), Formula (I-G), or Formula (I), R⁶ is hydrogen. In some embodiments of Formula (II), Formula (I-G), or Formula (I), R⁶ is halo. In some embodiments of Formula (II), Formula (I-G), or Formula (I), R⁶ is fluoro. In some embodiments of Formula (II), Formula (I-G), or Formula (I), R⁶ is chloro. In some embodiments of Formula (II), Formula (I-G), or Formula (I), R⁶ is bromo. In some embodiments of Formula (II), Formula (I-G), or Formula (I), R⁶ is iodo.

In some embodiments of a compound of Formula (II), p is 1. In some embodiments of a compound of Formula (II), p is 1, and the compound is of Formula (I-G). In other embodiments of a compound of Formula (II), p is 1, and the compound is of Formula (I).

In some embodiments of Formula (II), Formula (I-G) or Formula (I), R⁴ is selected from the group consisting of Z¹NR^{a}C(O)—, Z²C(O)NR^{b}—, Z³(CR^{c}R^{d})ₘNR^{e}—, Z⁴S(O)₂(CH₂)ₙ—, Z⁵OC(O)—, and NR^{f}R^{g}C(O)—. In some embodiments, R⁴ is Z¹NR^{a}C(O)— or NR^{f}R^{g}C(O)—. In some embodiments, R⁴ is Z¹NR^{a}C(O)— or Z²C(O)NR^{b}—.

In another aspect, the compound of Formula (II), Formula (I-G) or Formula (I) is a compound of Formula (I-A)

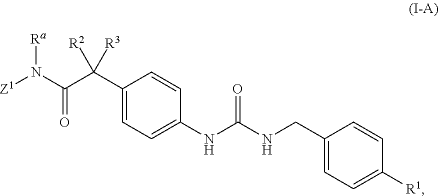

or a pharmaceutically acceptable salt thereof, wherein R¹, R², R³, R^{a}, and Z¹ are as defined for Formula (II), Formula (I-G), or Formula (I), or any variation or embodiment thereof.

In some embodiments, the compound is a compound of Formula (I-A1), (I-A2), (I-A3), or (I-A4)

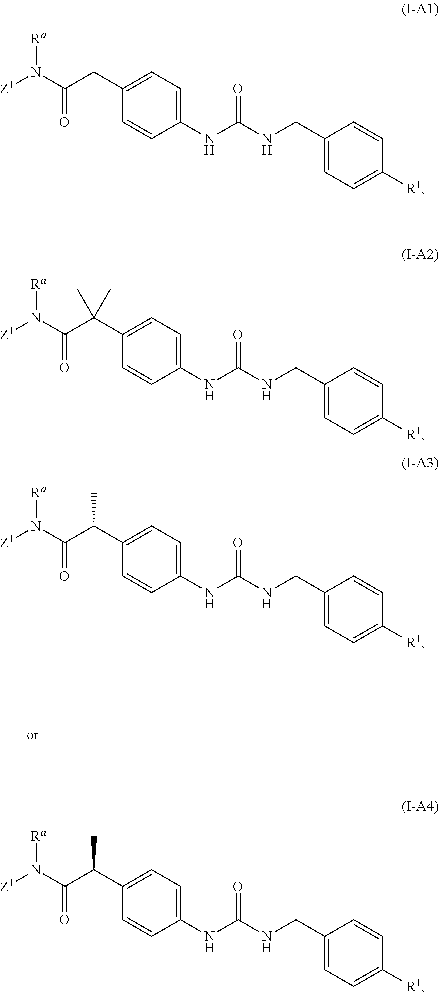

or a pharmaceutically acceptable salt thereof, wherein R¹, R^{a}, and Z¹ are as defined for Formula (II), Formula (I-G), Formula (I), or Formula (I-A), or any variation or embodiment thereof.

In some embodiments of Formula (II), Formula (I-G), Formula (I), or Formula (I-A), R^{a} is hydrogen. In some embodiments, R^{a} is C₁-C₆ alkyl. For example, in some embodiments, R^{a} is methyl, ethyl, propyl, isopropyl, n-butyl, sec-butyl, or tert-butyl.

In some embodiments of Formula (II), Formula (I-G), Formula (I), or Formula (I-A), Z¹ is R^{z}. In some embodiments, Z¹ is selected from the group consisting of:

C₁-C₆ alkyl optionally substituted with one or more substituents independently selected from the group consisting of —OH, C₃-C₆ cycloalkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of C₁-C₆ alkyl and C₁-C₆ alkoxy;

C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with C₁-C₆ alkyl; and

3- to 10-membered heterocycloalkyl or heterocycloalkenyl optionally substituted with one or more substituents independently selected from the group consisting of —C₁-C₆ alkyl and —C(O)OC₁-C₆ alkyl, wherein the —C₁-C₆ alkyl is optionally substituted with C₆-C₁₂ aryl.

In some embodiments of Formula (II), Formula (I-G), Formula (I), or Formula (I-A), Z¹ is C₁-C₆ alkyl. In some embodiments, Z¹ is unsubstituted C₁-C₆ alkyl. In some embodiments, Z¹ is C₁-C₆ alkyl optionally substituted with one or more substituents independently selected from the group consisting of —OH, C₃-C₆ cycloalkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of C₁-C₆ alkyl and C₁-C₆ alkoxy.

In some embodiments of Formula (II), Formula (I-G), Formula (I), or Formula (I-A), Z¹ is C₃-C₆ cycloalkyl. In some embodiments, Z¹ is unsubstituted C₃-C₆ cycloalkyl. In some embodiments, Z¹ is C₃-C₆ cycloalkyl substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with C₁-C₆ alkyl. In some embodiments, Z¹ is C₃-C₆ cycloalkyl optionally substituted with one or more groups independently selected from methoxy, ethoxy, and phenyl. In some embodiments, Z¹ is C₃-C₆ cycloalkyl optionally substituted with C₁-C₆ alkoxy optionally substituted with 5- or 10-membered heteroaryl, wherein the 5- or 10-membered heteroaryl is optionally further substituted with C₁-C₆ alkyl

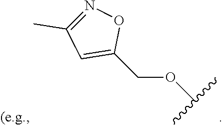 [SMILES: *OCc1cc(C)no1]

In some embodiments, Z¹ is C₃-C₆ cycloalkyl optionally substituted phenyl. In some embodiments, Z¹ is cyclopropyl, cyclobutyl, cyclopentyl, or cyclohexyl, each optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with C₁-C₆ alkyl.

In some embodiments of Formula (II), Formula (I-G), Formula (I), or Formula (I-A), Z¹ is 3- to 10-membered heterocycloalkyl or heterocycloalkenyl. In some embodiments, Z¹ is a 3- to 10-membered heterocycloalkyl or heterocycloalkenyl containing one or more heteroatoms independently selected from the group consisting of N, O, and S. In some embodiments, Z¹ is a 3- to 6-membered heterocycloalkyl or heterocycloalkenyl. In some embodiments, Z¹ is 3- to 10-membered heterocycloalkyl or heterocycloalkenyl optionally substituted with one or more substituents independently selected from the group consisting of —C₁-C₆ alkyl and —C(O)OC₁-C₆ alkyl, wherein the —C₁-C₆ alkyl is optionally substituted with C₆-C₁₂ aryl. In some embodiments, Z¹ is

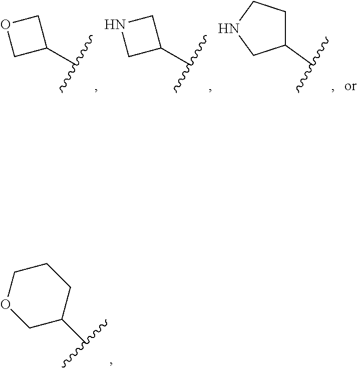 [SMILES: *C1COC1 ; *C1CNC1 ; *C1CCNC1 ; *C1CCCOC1]

each optionally substituted with one or more substituents independently selected from the group consisting of —C₁-C₆ alkyl and —C(O)OC₁-C₆ alkyl, wherein the —C₁-C₆ alkyl is optionally substituted with C₆-C₁₂ aryl.

In some embodiments of Formula (II), Formula (I-G), Formula (I), or Formula (I-A), Z¹ is C₁-C₆ alkyl. In certain embodiments, Z¹ is ethyl. In some embodiments, Z¹ is selected from the group consisting of ethyl,

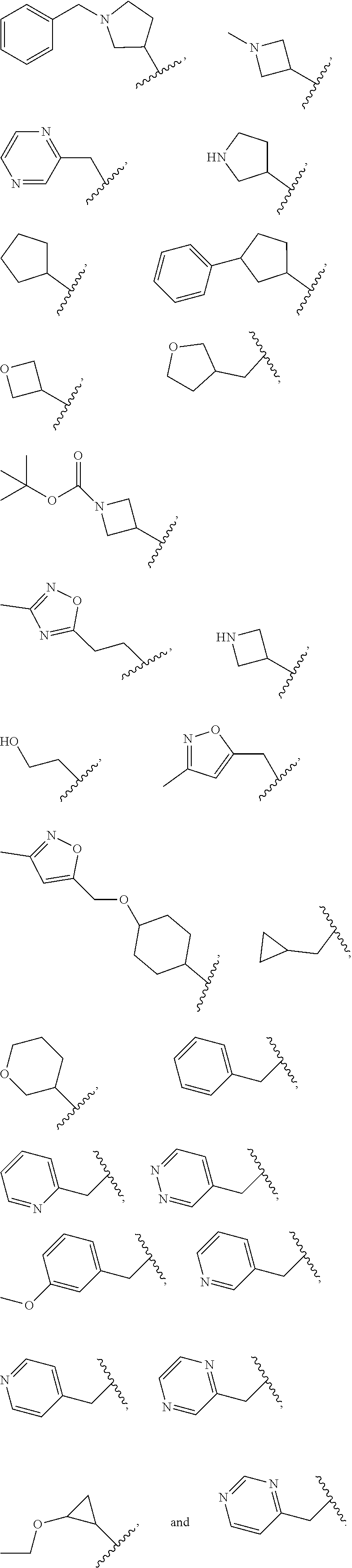

In another aspect, the compound of Formula (II), Formula (I-G), or Formula (I) is a compound of Formula (I-B)

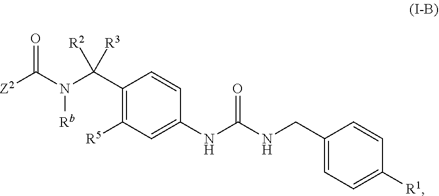

or a pharmaceutically acceptable salt thereof, wherein R¹, R², R³, R⁵, R^{b}, and Z² are as defined for Formula (II), Formula (I-G), or Formula (I), or any variation or embodiment thereof.

In some embodiments of Formula (II), Formula (I-G), Formula (I), or Formula (I-B), R^{b} is hydrogen. In some embodiments, R^{b} is C₁-C₆ alkyl. For example, in some embodiments, R^{b} is methyl, ethyl, propyl, isopropyl, n-butyl, sec-butyl, or tert-butyl. In some embodiments of Formula (II), Formula (I-G), Formula (I), or Formula (I-B), R^{s} is hydrogen. In other embodiments, R^{b} is taken together with R^{s} and the intervening atoms to form a 5- to 6-membered heterocycloalkyl or heterocycloalkenyl ring. In some embodiments of Formula (II) or Formula (I-G), R⁵ is halo. In some embodiments, R⁵ is fluoro. In some embodiments, R⁵ is chloro. In some embodiments, R⁵ is bromo. In some embodiments, R⁵ is iodo.

In some embodiments, the compound is a compound of Formula (I-B1), (I-B2), or (I-B3)

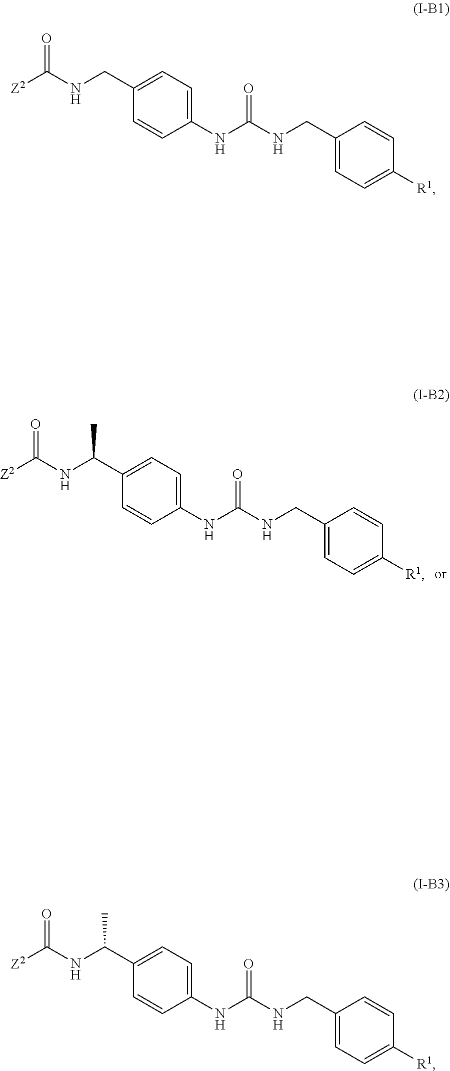

or a pharmaceutically acceptable salt thereof, wherein R¹ and Z² are as defined for Formula (II), Formula (I-G), Formula (I), or Formula (I-B), or any variation or embodiment thereof.

In some embodiments of Formula (II), Formula (I-G), Formula (I), or Formula (I-B), Z² is hydrogen. In some embodiments, Z² is R^{z}. In some embodiments, Z² is selected from the group consisting of

C₁-C₆ alkyl optionally substituted with one or more substituents independently selected from the group consisting of C₃-C₆ cycloalkyl and 5- to 10-membered heteroaryl;

C₃-C₆ cycloalkyl optionally substituted with one or more substituents independently selected from the group consisting of C₁-C₆ alkyl and C₁-C₆ alkoxy;

C₁-C₆ alkoxy;

3- to 10-membered heterocycloalkyl or heterocycloalkenyl optionally substituted with one or more independently selected —C₁-C₆ alkyl substituents;

C₆-C₁₂ aryl; and

5- to 10-membered heteroaryl optionally substituted with one or more independently selected C₁-C₆ alkyl substituents.

In some embodiments of Formula (II), Formula (I-G), Formula (I), or Formula (I-B), Z² is C₁-C₆ alkyl optionally substituted with one or more substituents independently selected from the group consisting of C₃-C₆ cycloalkyl and 5- to 10-membered heteroaryl. In some embodiments, Z² is methyl, ethyl, propyl, isopropyl, n-butyl, sec-butyl, or tert-butyl, each optionally substituted with one or more substituents independently selected from the group consisting of C₃-C₆ cycloalkyl and 5- to 10-membered heteroaryl.

In some embodiments of Formula (II), Formula (I-G), Formula (I), or Formula (I-B), Z² is C₃-C₆ cycloalkyl optionally substituted with one or more substituents independently selected from the group consisting of C₁-C₆ alkyl and C₁-C₆ alkoxy. In some embodiments, Z² is cyclopropyl, cyclobutyl, cyclopentyl, or cyclohexyl, each optionally substituted with one or more substituents independently selected from the group consisting of C₁-C₆ alkyl and C₁-C₆ alkoxy.

In some embodiments of Formula (II), Formula (I-G), Formula (I), or Formula (I-B), Z² is C₁-C₆ alkoxy. In some embodiments, Z² is methoxy, ethoxy, propoxy, isopropoxy, n-butoxy, sec-butoxy, or tert-butoxy.

In some embodiments of Formula (II), Formula (I-G), Formula (I), or Formula (I-B), Z² is a 3- to 10-membered heterocycloalkyl or heterocycloalkenyl optionally substituted with one or more independently selected —C₁-C₆ alkyl substituents. In some embodiments, Z² is a 4- to 6-membered heterocycloalkyl or heterocycloalkenyl optionally substituted with one or more independently selected —C₁-C₆ alkyl substituents. In some embodiments, Z² is an azetidinyl group optionally substituted with one or more —C₁-C₆ alkyl substituents or a tetrahydrofuranyl group optionally substituted with one or more independently selected —C₁-C₆ alkyl substituents. In some embodiments, Z² is

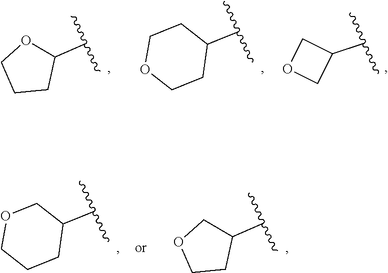 [SMILES: *C1CCCO1 ; *C1CCOCC1 ; *C1COC1 ; *C1CCCOC1 ; *C1CCOC1]

each optionally substituted with one or more independently selected —C₁-C₆ alkyl substituents. In some embodiments, Z² is

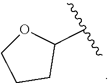 [SMILES: *C1CCCO1]

In some embodiments, Z² is

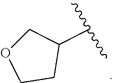 [SMILES: *C1CCOC1]

In some embodiments, Z² is

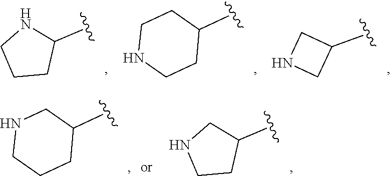

each optionally substituted with one or more independently selected —C₁-C₆ alkyl substituents. In some embodiments, Z² is

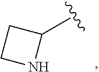

optionally substituted with one or more independently selected —C₁-C₆ alkyl substituents. In some embodiments, Z² is

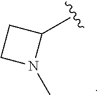

In some embodiments of Formula (II), Formula (I-G), Formula (I), or Formula (I-B), Z² is C₆-C₁₂ aryl. For instance, in some embodiments, Z² is phenyl or naphthyl.

In some embodiments of Formula (II), Formula (I-G), Formula (I), or Formula (I-B), Z² is 5- to 10-membered heteroaryl optionally substituted with one or more independently selected C₁-C₆ alkyl substituents. In some embodiments, Z² is a 5- to 6-membered heteroaryl optionally substituted with one or more independently selected —C₁-C₆ alkyl substituents. In some embodiments, Z² is a pyridyl group optionally substituted with one or more independently selected —C₁-C₆ alkyl substituents. In some embodiments, Z² is a pyridyl group optionally substituted with methyl, ethyl, or isopropyl. In some embodiments, Z² is a pyridyl group substituted with methyl. In other embodiments, Z² is a pyridyl group substituted with isopropyl. In some embodiments, Z² is selected from the group consisting of

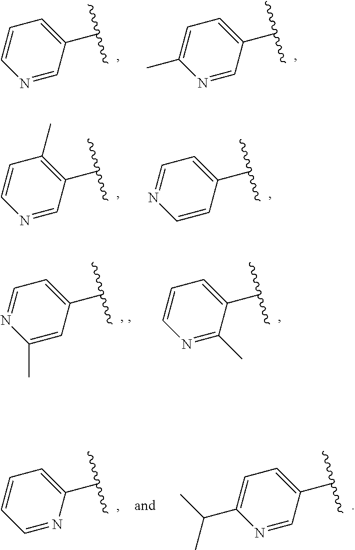

In some embodiments, Z² is

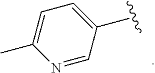

In some embodiments, Z² is selected from the group consisting of ethyl,

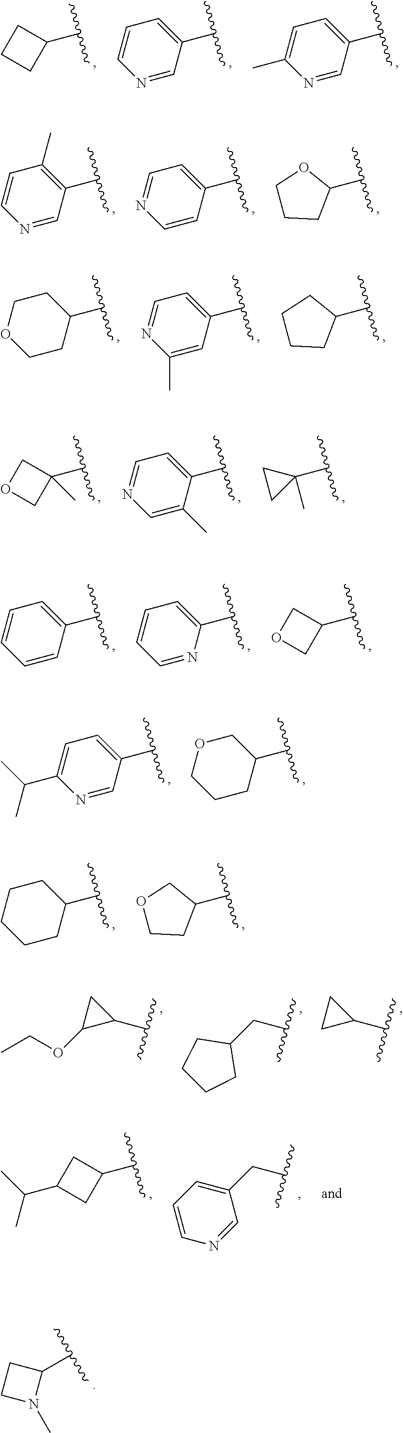

In some embodiments, Z² is

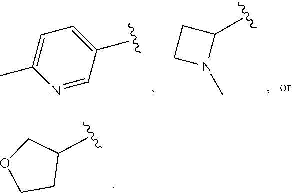

In another aspect, the compound of Formula (II), Formula (I-G), or Formula (I) is a compound of Formula (I-C).

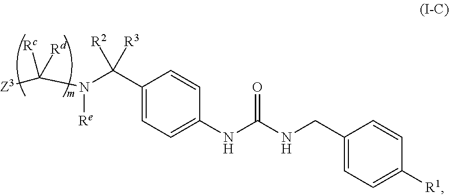

or a pharmaceutically acceptable salt thereof, wherein R¹, R², R³, R^{e}, R^{d}, R^{e}, m, and Z³ are as defined for Formula (I-G) or Formula (I), or any variation or embodiment thereof.

In some embodiments of Formula (II), Formula (I-G), Formula (I), or Formula (I-C), m is 0. In other embodiments, m is 1. In some embodiments of Formula (I-G), Formula (I), or Formula (I-C), R^{c} is hydrogen. In other embodiments, R^{e} is C₁-C₆ alkyl. In some embodiments of Formula (II), Formula (I-G), Formula (I), or Formula (I-C), R^{d} is hydrogen. In other embodiments, R^{d} is C₁-C₆ alkyl. In some embodiments, R^{e} and R^{d} together with the carbon to which they are attached form a C₃-C₆ cycloalkyl.

In some embodiments, the compound is a compound of Formula (I-C1), (I-C2), (I-C3), or (I-C4)

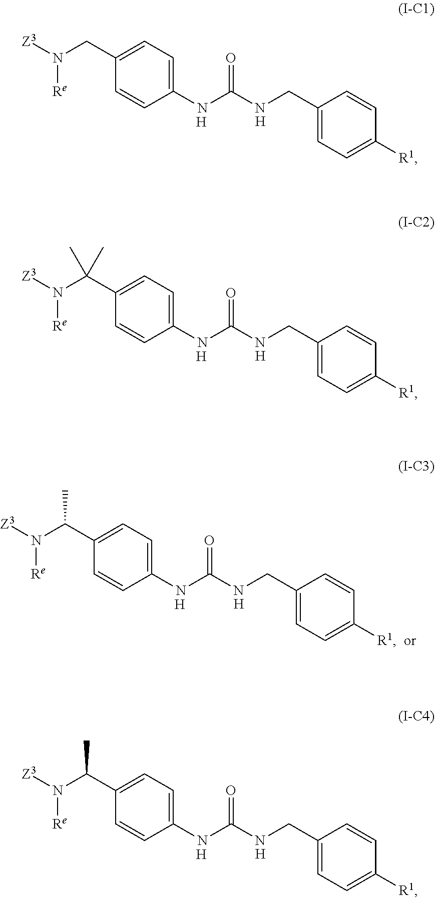

or a pharmaceutically acceptable salt thereof, wherein R¹, R^{e}, and Z³ are as defined for Formula (II), Formula (I-G), Formula (I), or Formula (I-C), or any variation or embodiment thereof.

In some embodiments of Formula (II), Formula (I-G), Formula (I), or Formula (I-C), R^{e} is hydrogen. In other embodiments, R^{e} is C₁-C₆ alkyl.

In some embodiments of Formula (II), Formula (I-G), Formula (I), or Formula (I-C), Z³ is hydrogen. In some embodiments, Z³ is R^{z}. In some embodiments, Z³ is selected from the group consisting of C₃-C₆ cycloalkyl; 3- to 10-membered heterocycloalkyl or heterocycloalkenyl optionally substituted with —C₁-C₆ alkyl or oxo; C₆-C₁₂ aryl; and 5- to 10-membered heteroaryl optionally substituted with one or more independently selected C₁-C₆ alkyl substituents. In some embodiments, Z³ is 3- to 10-membered heterocycloalkyl or heterocycloalkenyl optionally substituted with —C₁-C₆ alkyl or oxo. In some embodiments, Z³ is selected from the group consisting of

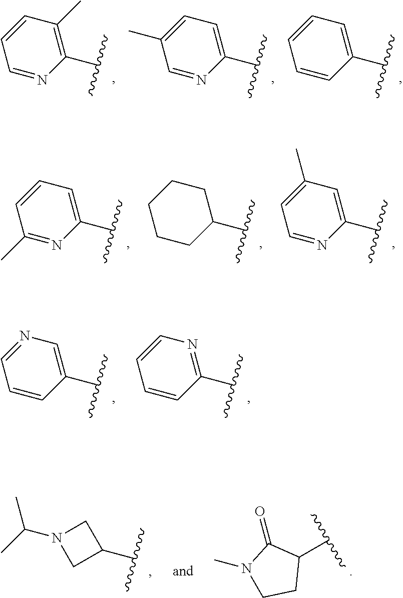

In some embodiments, Z³ is

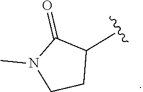

In another aspect, the compound of Formula (II), Formula (I-G), or Formula (I) is a compound of Formula (ID)

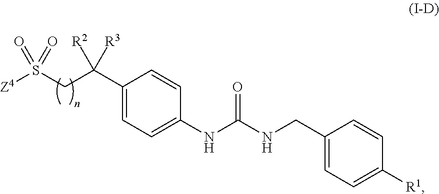

or a pharmaceutically acceptable salt thereof, wherein R¹, R², R³, n, and Z⁴ are as defined for Formula (II), Formula (I-G) or Formula (I) or any variation or embodiment thereof.

In some embodiments of Formula (II), Formula (I-G), Formula (I), or Formula (I-D), n is 0. In some embodiments, n is 1. In other embodiments, n is 2.

In some embodiments, the compound is a compound of Formula (I-D1) or (I-D2)

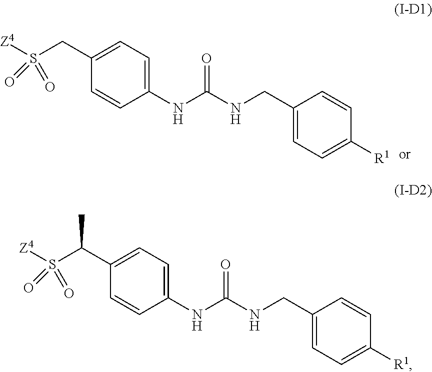

or a pharmaceutically acceptable salt thereof, wherein R¹ and Z⁴ are as defined for Formula (I-G), Formula (I), or Formula (I-D), or any variation or embodiment thereof.

In some embodiments, the compound is a compound of Formula (I-D3), (I-D4), (I-D5), (I-D6) or (I-D7)

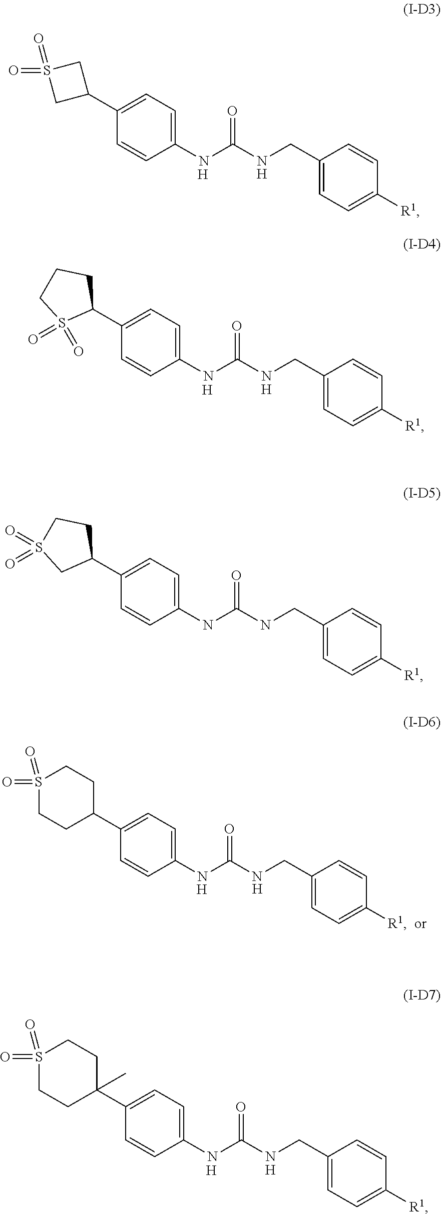

or a pharmaceutically acceptable salt thereof, wherein R¹ is as defined for Formula (II), Formula (I-G), Formula (I), or Formula (I-D), or any variation or embodiment thereof.

In some embodiments of Formula (II), Formula (I-G), Formula (I), or Formula (I-D), Z⁴ is hydrogen. In some embodiments, Z⁴ is R^{z}. In other embodiments, Z⁴ is C₁-C₆ alkyl. For example, in some embodiments, Z⁴ is methyl, ethyl, propyl, isopropyl, n-butyl, sec-butyl, or tert-butyl. In some embodiments, Z⁴ is is taken together with R² and the intervening atoms to form a 4-6 membered heterocycloalkyl or heterocycloalkenyl ring. In some embodiments,

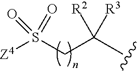

is selected from the group consisting of

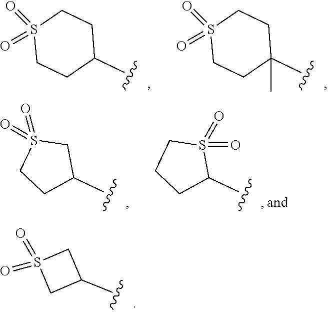

In another aspect, the compound of Formula (II), Formula (I-G), or Formula (I) is a compound of Formula (IE)

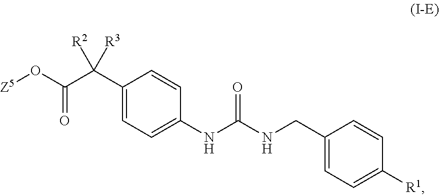

or a pharmaceutically acceptable salt thereof, wherein R¹, R², R³, and Z⁵ are as defined for Formula (II), Formula (I-G), Formula (I) or any variation or embodiment thereof.

In some embodiments of Formula (II), Formula (I-G), Formula (I), or Formula (I-E), Z⁵ is C₁-C₆ alkyl. For example, in some embodiments, Z⁵ is methyl, ethyl, propyl, isopropyl, n-butyl, sec-butyl, or tert-butyl. In some embodiments, Z⁵ is ethyl.

In another aspect, the compound of Formula (II), Formula (I-G), or Formula (I) is a compound of Formula (IF)

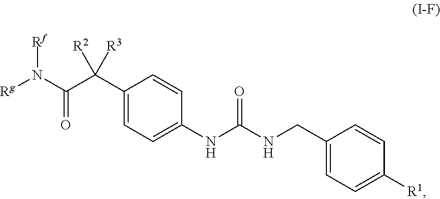

or a salt thereof, wherein R¹, R², R³, R^{f}, and R⁹ are as defined for Formula (II), Formula (I-G), or Formula (I), or any variation or embodiment thereof.

In some embodiments of Formula (II), Formula (I-G), Formula (I), or Formula (I-F), R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl.

In some embodiments of Formula (II), Formula (I-G), Formula (I), or Formula (I-F), R^{f} and R^{g} together with the nitrogen to which they are attached form a 3- to 6-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl. In some embodiments, R^{f} and R^{g} together with the nitrogen to which they are attached form a 3- to 6-membered heterocycloalkyl or heterocycloalkenyl selected from the group consisting of azetidinyl, pyrrolidinyl, and piperidinyl, each optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl. In some embodiments,

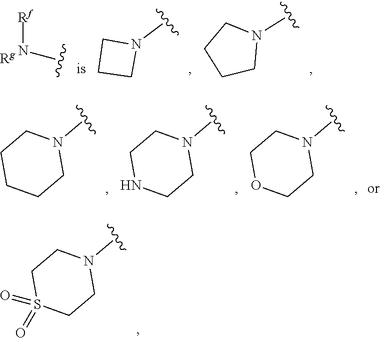

each optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl. In some embodiments, R^{f} and R^{g} together with the nitrogen to which they are attached form a 5- to 6-membered heterocycloalkyl or heterocycloalkenyl optionally substituted with —C₁-C₆ alkyl, wherein the —C₁-C₆ alkyl is optionally substituted with —OH. In some embodiments, R^{f} and R^{g} together with the nitrogen to which they are attached form a pyrrolidinyl optionally substituted with —C₁-C₆ alkyl, wherein the —C₁-C₆ alkyl is optionally substituted with —OH. In some embodiments,

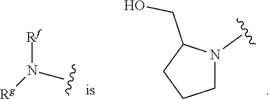

In some embodiments of Formula (II), Formula (I-G), Formula (I), or Formula (I-F), R^{f} and R^{g} together with the nitrogen to which they are attached form a 6- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl. In some embodiments, R^{f} and R^{g} together with the nitrogen to which they are attached form a bicyclic 6- to 10-membered heterocycloalkyl or heterocycloalkenyl. For instance, in some embodiments R^{f} and R^{g} together with the nitrogen to which they are attached form

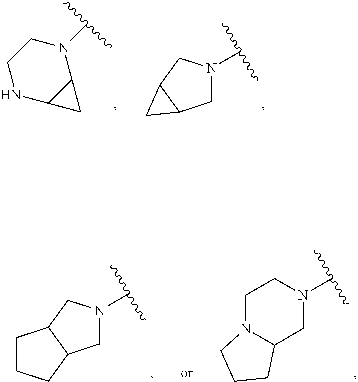 [SMILES: *N1CCNC2CC21 ; *N1CC2CC2C1 ; *N1CC2CCCC2C1 ; *N1CCN2CCCC2C1]

each optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl. In some embodiments, R^{f} and R^{g} together with the nitrogen to which they are attached form a bridged 6- to 10-membered heterocycloalkyl or heterocycloalkenyl. For instance, in some embodiments,

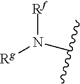

is selected from the group consisting of

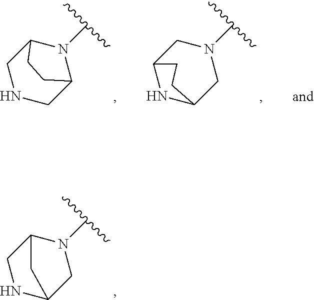 [SMILES: *N1C2CCC1CNC2 ; *N1CC2CCC(C1)N2 ; *N1CC2CC1CN2]

each optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl.

In some embodiments, R^{f} and R^{g} together with the nitrogen to which they are attached form a spirocyclic 6- to 10-membered heterocycloalkyl or heterocycloalkenyl. For instance, in some embodiments

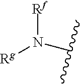

is selected from the group consisting of

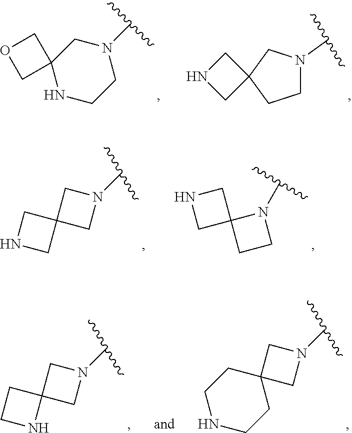 [SMILES: *N1CCNC2(COC2)C1 ; *N1CCC2(CNC2)C1 ; *N1CC2(CNC2)C1 ; *N1CCC12CNC2 ; *N1CC2(CCN2)C1 ; *N1CC2(CCNCC2)C1]

each optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl.

In some embodiments,

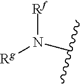

is selected from the group consisting of

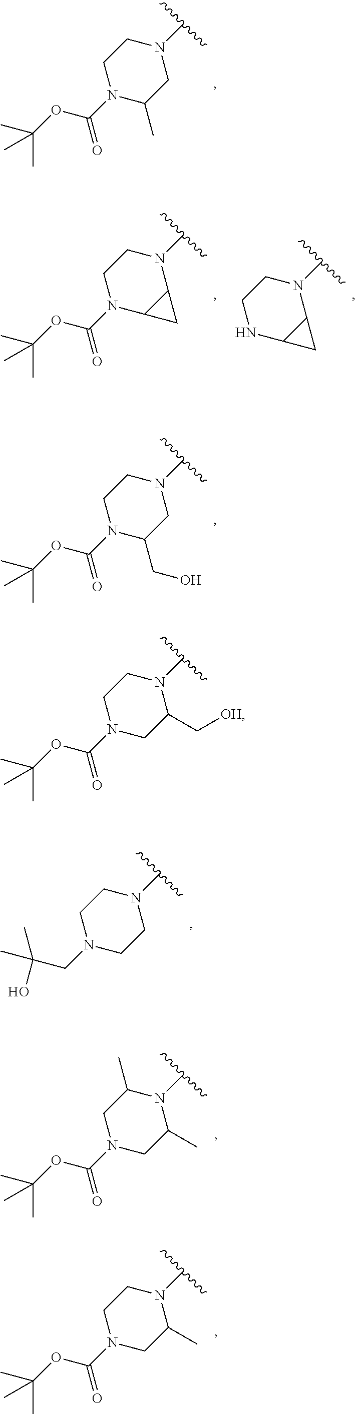

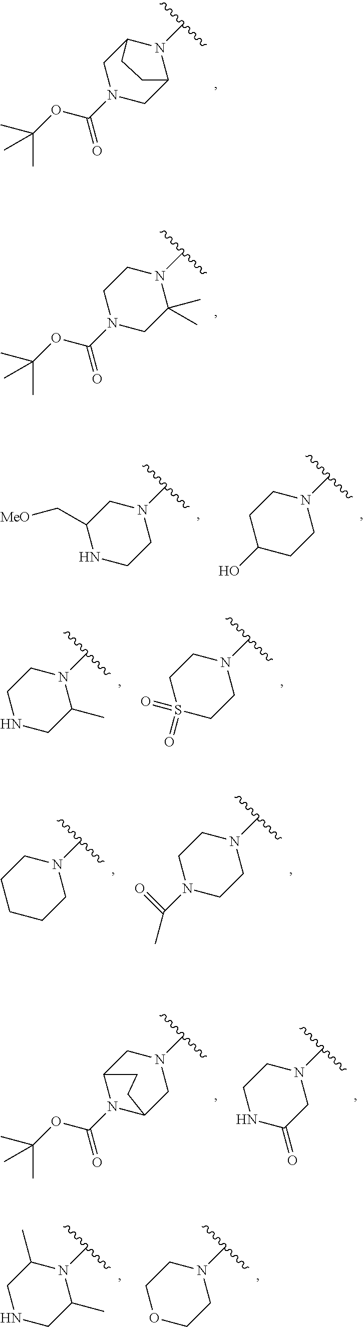

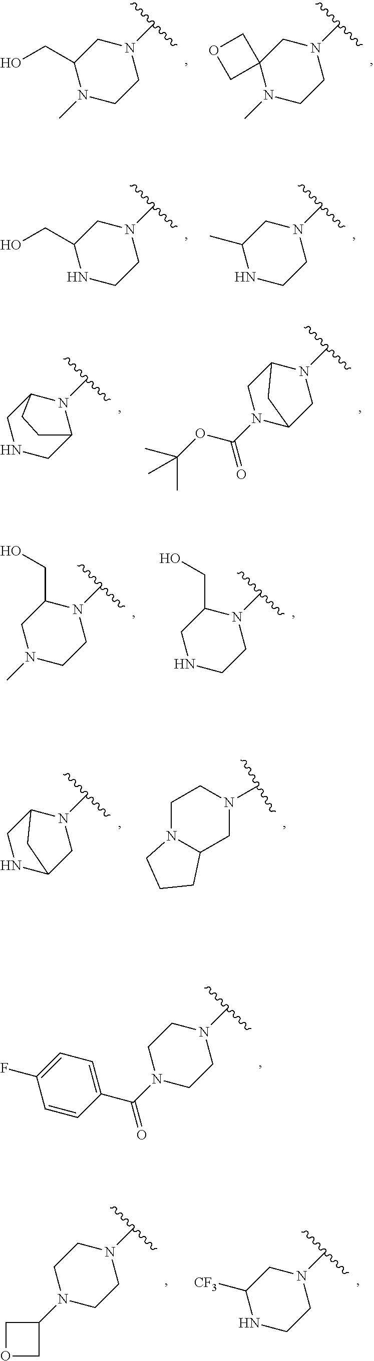

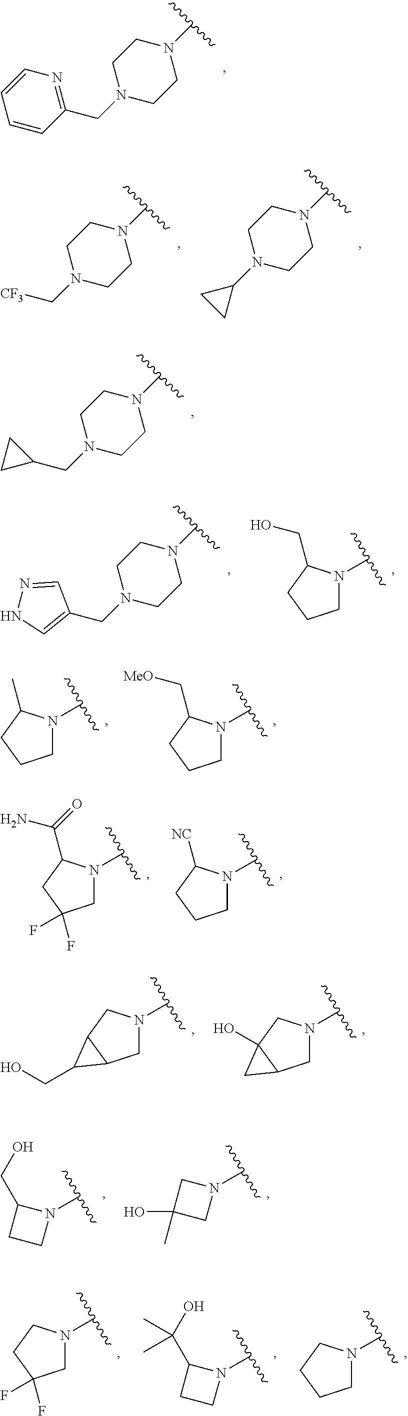

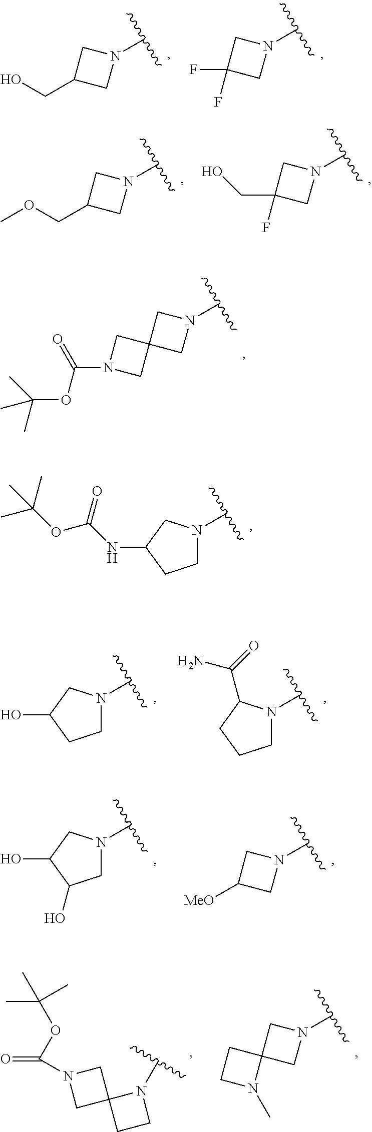

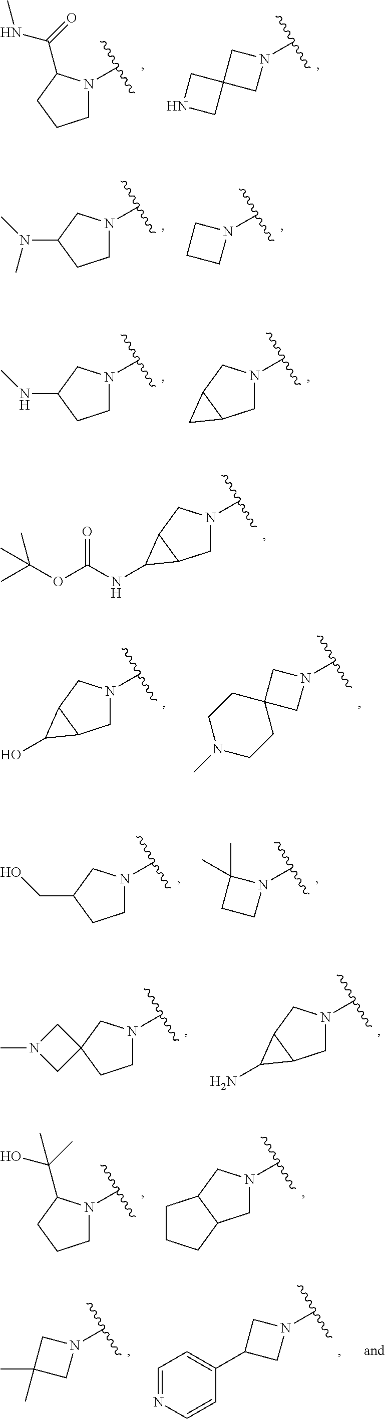

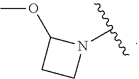 [SMILES: *N1CCC1OC]

In some embodiments of Formula (II), Formula (I-G), or Formula (I), R⁴ is a 5- to 10 membered heteroaryl optionally substituted with one or more independently selected C₁-C₆ alkyl substituents. In some embodiments, R⁴ is selected from the group consisting of pyridyl, quinolinyl, isoquinolinyl, quinoxalinyl, cinnolinyl, quinazolinyl, naphthyridinyl, benzoxazolyl, benzothiazolyl, benzoimidazoyl, pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridazinyl, pyrimidinyl, pyrazinyl, triazinyl, tetrazinyl, indolyl, isoindolyl, indazolyl, benzotriazolyl, benzofuranyl, benzoisoxazolyl, benzoxadiazolyl, benzothiophenyl, benzoisothiazolyl, benzothiadiazolyl, pyrrolopyridinyl, pyrazolopyridinyl, imidazopyridinyl, triazolopyridinyl, furopyridinyl, oxazolopyridinyl, isoxazolopyridinyl, oxadiazolopyridinyl, thienopyridinyl, thiazolopyridinyl, isothiazolopyridinyl, thiadiazolopyridinyl, thienopyridinyl, phthalazinyl, pyrazolothiazolyl, pyrazolothiazolyl and imidazothiazolyl, each optionally substituted with one or more independently selected C₁-C₆ alkyl substituents. In some embodiments, R⁴ is a 5- to 6 membered heteroaryl optionally substituted with one or more independently selected C₁-C₆ alkyl substituents. In some embodiments, R⁴ is pyrazolyl, pyridinyl, or oxadiazole, each optionally substituted with one or more independently selected C₁-C₆ alkyl substituents. In certain embodiments, R⁴ is selected from the group consisting of

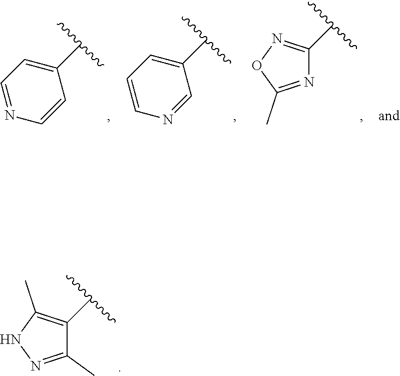

In some embodiments of Formula (II), Formula (I-G), or Formula (I), R⁴ is a 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents. In some embodiments, R⁴ is a 4- to 6-membered heterocycloalkyl or heterocycloalkenyl optionally substituted with —S(O)₂—C₁-C₆ alkyl or —C₁-C₆ alkyl optionally substituted with —OH. In some embodiments, R⁴ is an azetidinyl or piperazinyl optionally substituted with —S(O)₂—C₁-C₆ alkyl or —C₁-C₆ alkyl optionally substituted with —OH. In some embodiments, R⁴ is an azetidinyl optionally substituted with —S(O)₂—C₁-C₆ alkyl. In some embodiments, R⁴ is azetidinyl substituted with —S(O)₂CH₃. In some embodiments, R⁴ is a piperazinyl optionally substituted with —C₁-C₆ alkyl optionally substituted with —OH. In certain embodiments, R⁴ is a piperazinyl optionally substituted with —CH₂C(CH₃)₂OH.

In some embodiments of Formula (II), Formula (I-G), or Formula (I), R⁴ is selected from the group consisting of

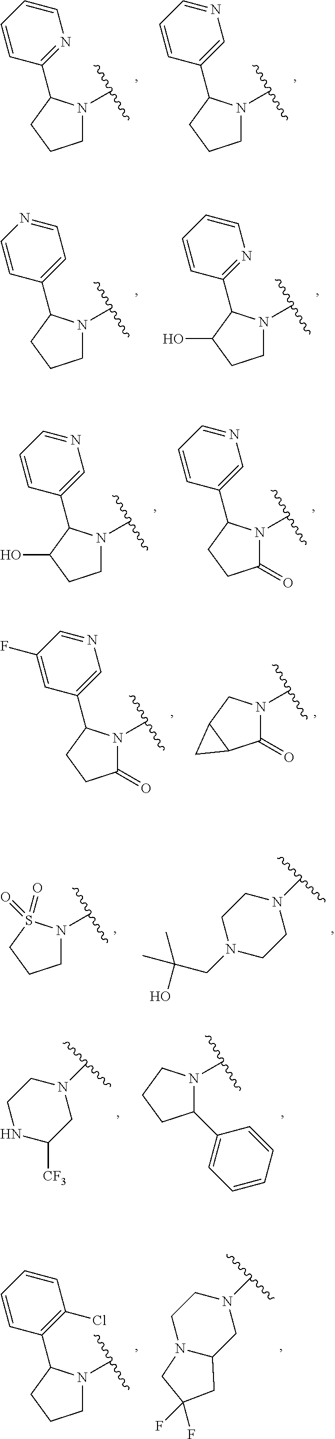

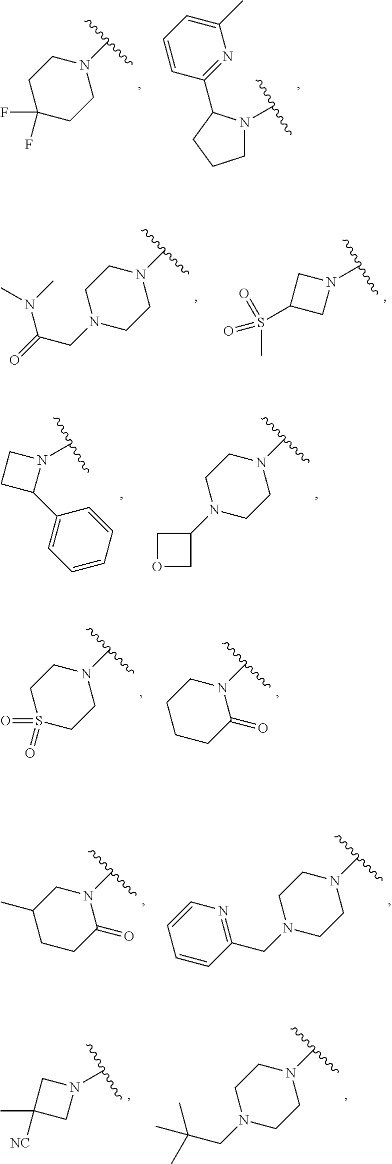

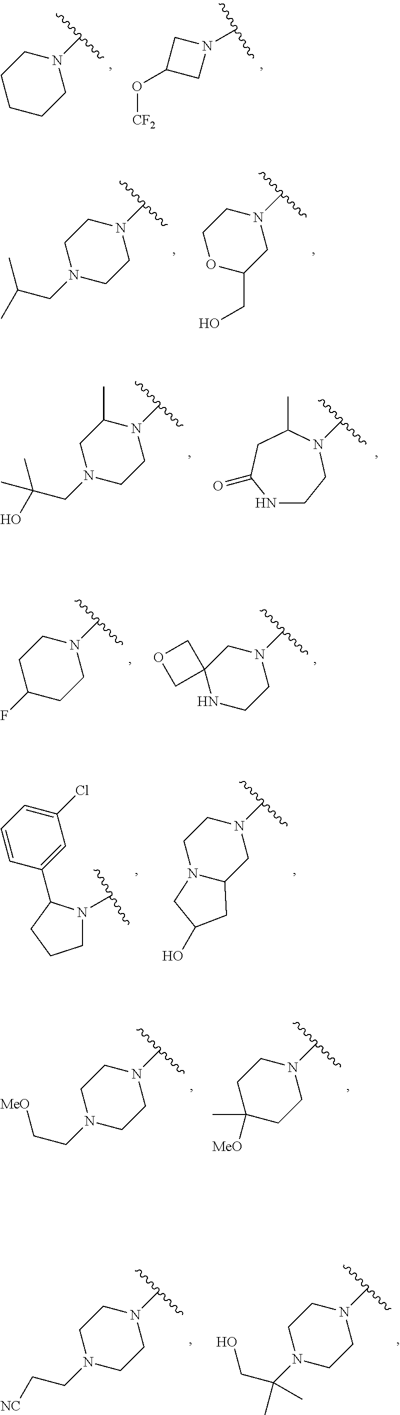

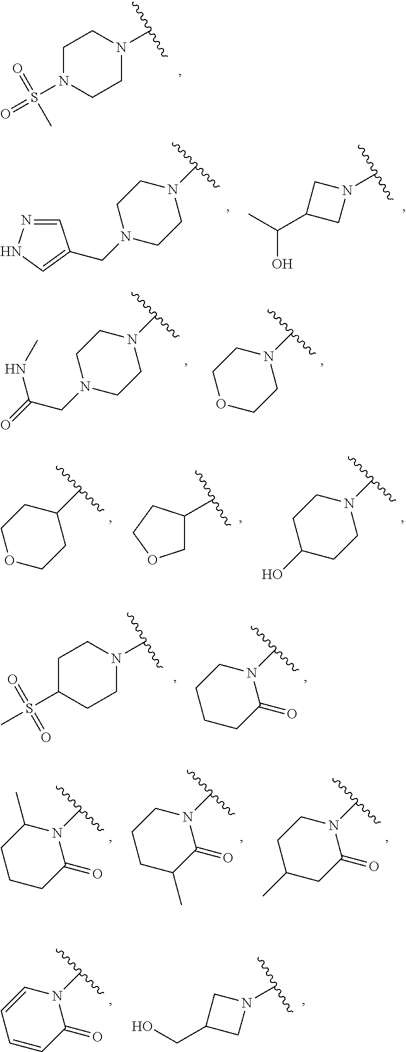

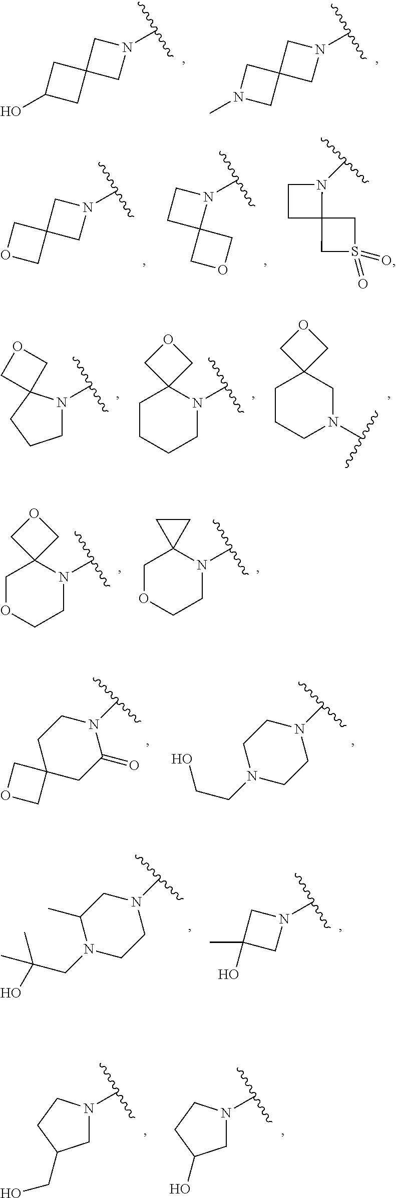

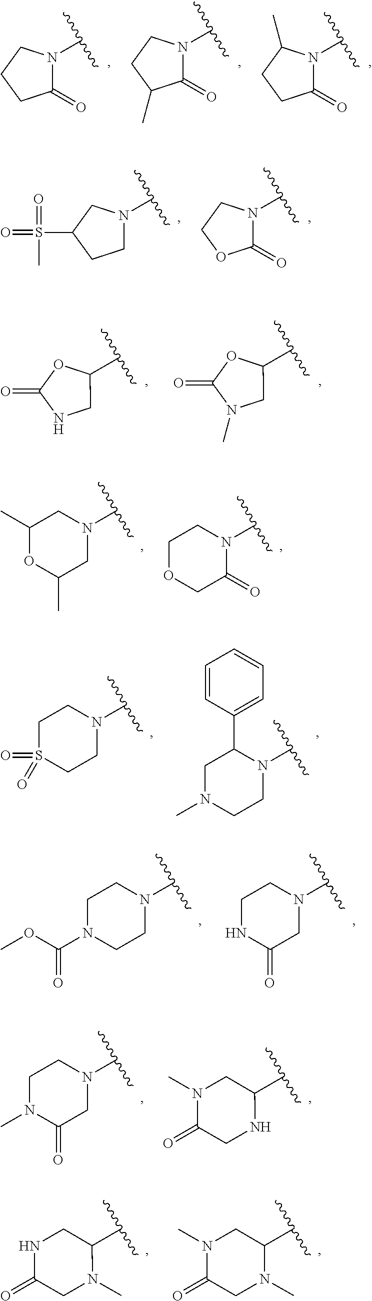

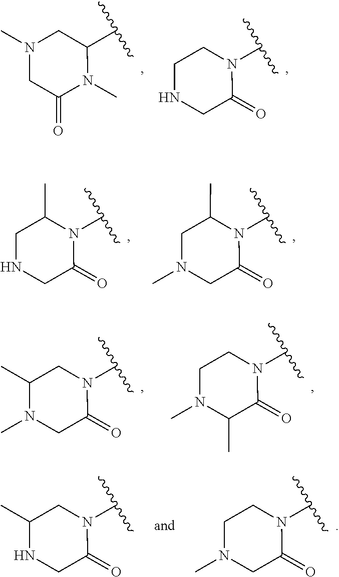

In some embodiments, R⁴ is selected from the group consisting of

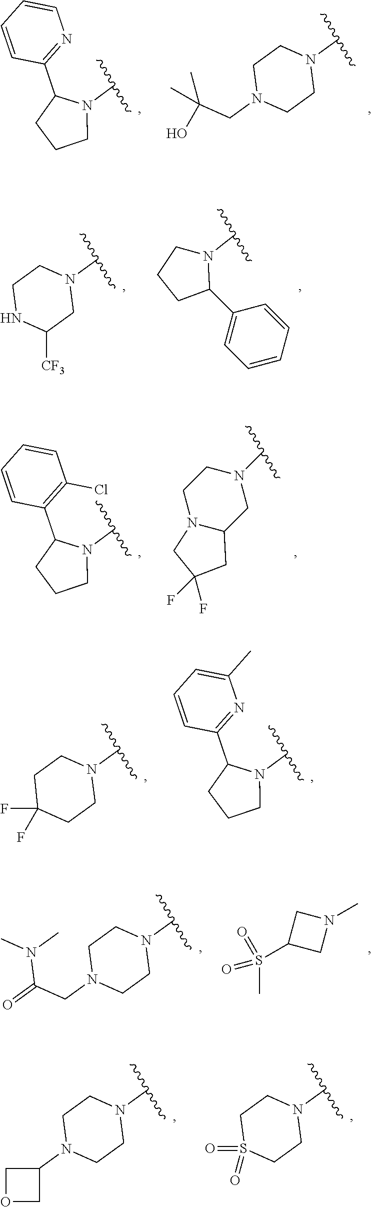

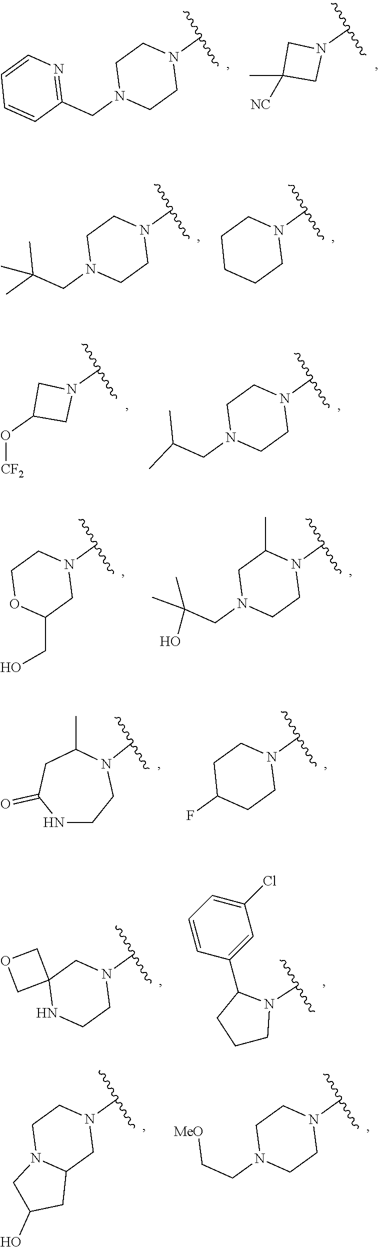

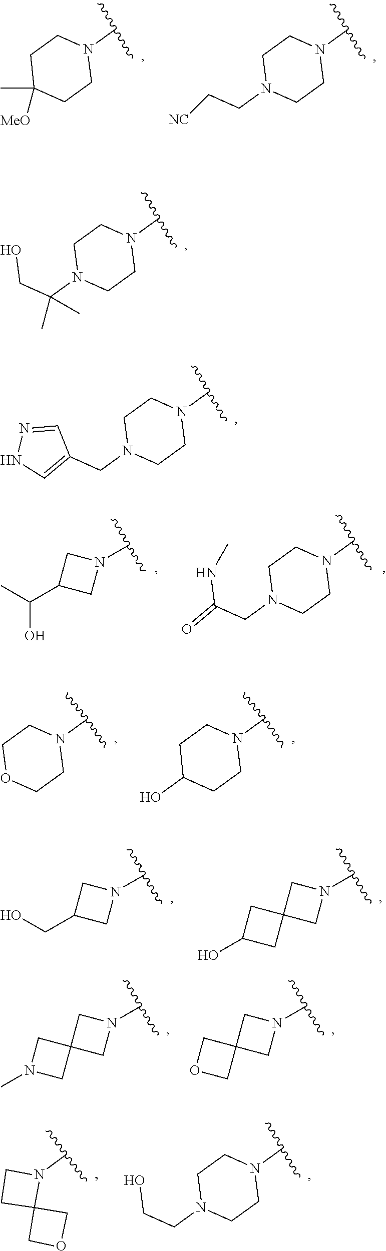

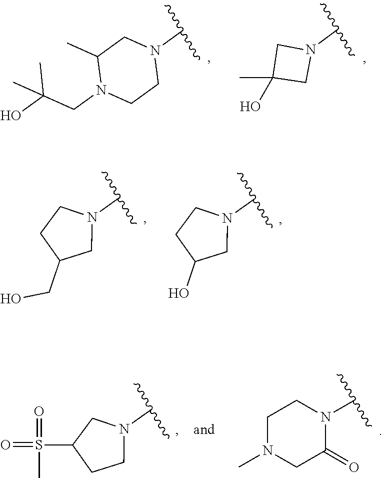

In some embodiments, R⁴ is

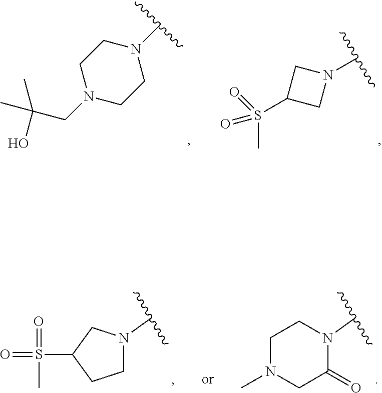

In some embodiments, R⁴ is

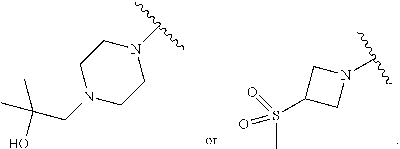

In some embodiments of Formula (II), Formula (I-G), or Formula (I), R⁴ is Z⁶S(O)₂N(R^{s})—. In some embodiments, Z⁶ is 5- to 6-membered heterocycloalkyl or heterocycloalkenyl. In other embodiments, Z⁶ is 5- to 6-membered heteroaryl. In some embodiments, Z⁶ is C₁-C₆ alkyl. In some embodiments, Z⁶ is methyl. In some embodiments of the foregoing, R^{s} is hydrogen. In other embodiments, R^{s} is C₁-C₆ alkyl. In still other embodiments, R^{s} is methyl. In some embodiments, R⁴ is

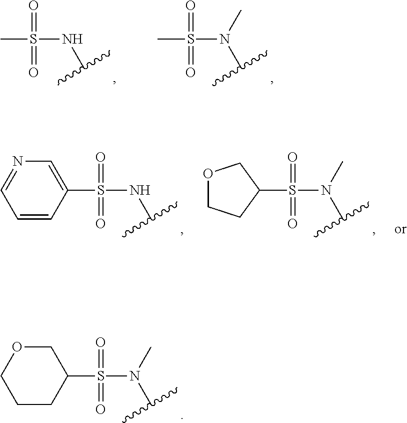

In some embodiments of Formula (II), Formula (I-G), or Formula (I), R⁴ is Z⁷N(R^{t})S(O)₂—. In some embodiments, Z⁷ is C₆-C₁₂ aryl. In some embodiments, Z⁷ is phenyl. In some embodiments, R^{t} is hydrogen. In other embodiments, R^{t} is C₁-C₆ alkyl. In still other embodiments, R^{t} is methyl. In some embodiments, R⁴ is —S(O)₂—NH-phenyl.

In some embodiments of Formula (II), Formula (I-G), or Formula (I), R⁴ is Z⁸—O—(CH₂)_{q}—. In some embodiments, q is 0, such that R⁴ is Z⁸—O—. In other embodiments, q is 1, such that R⁴ is Z⁸—O—(CH₂)—. In some embodiments of the foregoing, Z⁸ is 5- to 6-membered heteroaryl. In some embodiments, Z⁸ is pyridinyl. In other embodiments of the foregoing, Z⁸ is C₃-C₆ cycloalkyl. In some embodiments, Z⁸ is cyclopentyl. In some embodiments, R⁴ is

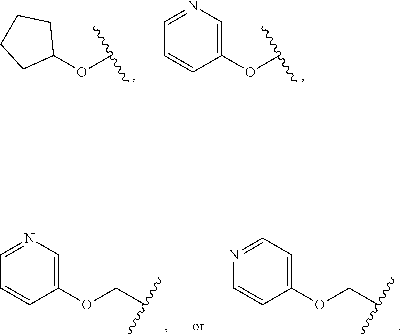 [SMILES: *OC1CCCC1 ; *Oc1cccnc1 ; *COc1cccnc1 ; *COc1ccncc1]

In some embodiments of Formula (II), p is 0. In some embodiments of Formula (II), p is 0, and the compound is of Formula (II-A)

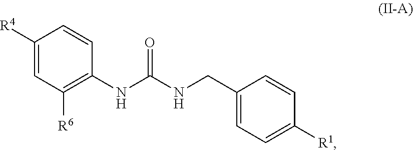

or a pharmaceutically acceptable salt thereof, wherein:
R¹ is halo or methoxy;

### R⁴ is

l) 3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents,
m) 3- to 6-membered heterocycloalkyl or heterocycloalkenyl comprising exactly one annular heteroatom, which is an oxygen atom, wherein the 3- to 6-membered heterocycloalkyl or heterocycloalkenyl is optionally substituted with one or more independently selected oxo or —C₁-C₆ alkyl substituents,
n) 3- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents,
o) 5-membered heterocycloalkyl or heterocycloalkenyl comprising exactly two annular heteroatoms, one of which is a nitrogen atom and the other of which is an oxygen atom, wherein the 5-membered heterocycloalkyl or heterocycloalkenyl is optionally substituted with one or more independently selected oxo, —C₁-C₆ alkyl, or —S(O)₂—(C₁-C₆ alkyl) substituents,
p) 6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, —C₁-C₆ alkyl, or —S(O)₂—(C₁-C₆ alkyl) substituents,
q) 5-membered heteroaryl comprising exactly two annular heteroatoms, one of which is a nitrogen atom and the other of which is an oxygen atom, wherein the 5-membered heteroaryl is substituted with exactly one methyl substituent,
r) 5-membered heteroaryl comprising exactly two annular heteroatoms, both of which are nitrogen atoms, wherein the 5-membered heteroaryl is substituted with one or more methyl substituents,
s) 6-membered heteroaryl comprising one or two annular heteroatoms and optionally substituted with one or more methyl substituents, wherein the 6-membered heteroaryl is other than

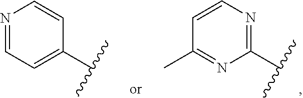

t) Z⁹—S(O)₂—,
u) Z¹⁰—S(O)₂—NH—,

### v) Z¹¹—C(O)—NH—,

w) Z¹²—CH₂—O—,

### x) Z¹³—O—,

y) Z¹⁴—C(H)(C₁-C₆ alkyl)-NH—C(O)—,
z)

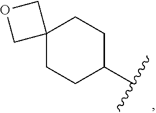 [SMILES: *C1CCC2(CC1)COC2]

or
aa)

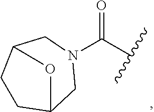

wherein

Z⁹ is selected from the group consisting of cyclopropyl, C₆-C₁₂ aryl, 3- to 10-membered heterocycloalkyl or hetercycloalkenyl optionally substituted with one or more independently selected R^{A} substituents, —NH(C₁-C₆ alkyl), —NH₂ substituted with one or more independently selected R^{B} substituents, and C₁-C₆ alkyl optionally substituted with one or more independently selected R^{C} substituents, provided that Z⁹ is other than

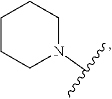 [SMILES: *N1CCCCC1]

unsubstituted methyl, or unsubstituted ethyl, wherein:
R^{A} is —C₁-C₆ alkyl or —CN; and
R^{B} is (i) —C₁-C₆ alkyl-(5- to 10-membered heteroaryl), or (ii) 5- to 10-membered heteroaryl optionally substituted with one or more independently selected C₆-C₁₂ aryl; and
R^{C} is 3- to 8-membered heterocycloalkyl or heterocycloalkenyl;

Z¹⁰ is C₁-C₆ alkyl substituted with one or more independently selected C₆-C₁₂ aryl substituents;

Z¹¹ is selected from the group consisting of C₃-C₁₀ cycloalkyl and C₁-C₆ alkyl substituted with one or more independently selected 3- to 10-membered heterocycloalkyl or hetercycloalkenyl substituents, provided that, when Z¹¹ is cyclopropyl, then R¹ is other than methoxy;

Z¹² is selected from the group consisting of C₆-C₁₂ aryl, 5- to 10-membered heteroaryl, 3- to 10-membered heterocycloalkyl or heterocycloalkenyl, C₁-C₆ alkyl substituted with one or more independently selected 3- to 10-membered heterocycloalkyl or hetercycloalkenyl substituents or 5- to 10-membered heteroaryl substituents, and —C(O)-(3- to 10-membered heterocycloalkyl or heterocycloalkenyl);

Z¹³ is 5- to 10-membered heteroaryl substituted with one or more independently selected —C(O)—NH(C₁-C₆ alkyl) substituents; and

Z¹⁴ is 5- to 10-membered heteroaryl optionally substituted with one or more independently selected C₁-C₆ alkyl substituents; and

R⁶ is hydrogen or halo.

In some embodiments of Formula (II) or Formula (II-A), R⁴ is selected from the group consisting of:

3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents,

3- to 6-membered heterocycloalkyl or heterocycloalkenyl comprising exactly one annular heteroatom, which is an oxygen atom, wherein the 3- to 6-membered heterocycloalkyl or heterocycloalkenyl is optionally substituted with one or more independently selected oxo or —C₁-C₆ alkyl substituents,

3- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents,

5-membered heterocycloalkyl or heterocycloalkenyl comprising exactly two annular heteroatoms, one of which is a nitrogen atom and the other of which is an oxygen atom, wherein the 5-membered heterocycloalkyl or heterocycloalkenyl is optionally substituted with one or more independently selected oxo, —C₁-C₆ alkyl, or —S(O)₂—(C₁-C₆ alkyl) substituents, and

6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, —C₁-C₆ alkyl, or —S(O)₂—(C₁-C₆ alkyl) substituents.

In some embodiments of Formula (II) or Formula (II-A), R⁴ is 3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents. In some embodiments, R⁴ is 5- to 6-membered heterocycloalkyl or heterocycloalkenyl comprising exactly two annular heteroatoms, both of which are nitrogen atoms, wherein the 5- to 6-membered heterocycloalkyl or heterocycloalkenyl is substituted with one or more independently selected —C₁-C₆ alkyl substituents and is optionally further substituted with one or more oxo substituents.

In some embodiments of Formula (II) or Formula (II-A), R⁴ is 3- to 6-membered heterocycloalkyl or heterocycloalkenyl comprising exactly one annular heteroatom, which is an oxygen atom, wherein the 3- to 6-membered heterocycloalkyl or heterocycloalkenyl is optionally substituted with one or more independently selected oxo or —C₁-C₆ alkyl substituents. In some embodiments, R⁴ is 5- to 6-membered heterocycloalkyl or heterocycloalkenyl comprising exactly one annular heteroatom, which is an oxygen atom, wherein the 5- to 6-membered heterocycloalkyl or heterocycloalkenyl is optionally substituted with one or more independently selected oxo or —C₁-C₆ alkyl substituents.

In some embodiments of Formula (II) or Formula (II-A), R⁴ is 3- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents. In some embodiments, R⁴ is 5- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents.

In some embodiments of Formula (II) or Formula (II-A), R⁴ is 5-membered heterocycloalkyl or heterocycloalkenyl comprising exactly two annular heteroatoms, one of which is a nitrogen atom and the other of which is an oxygen atom, wherein the 5-membered heterocycloalkyl or heterocycloalkenyl is optionally substituted with one or more independently selected oxo, —C₁-C₆ alkyl, or —S(O)₂—(C₁-C₆ alkyl) substituents.

In some embodiments of Formula (II) or Formula (II-A), R⁴ is 6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, —C₁-C₆ alkyl, or —S(O)₂—(C₁-C₆ alkyl) substituents.

In some embodiments of Formula (II) or Formula (II-A), R⁴ is selected from the group consisting of

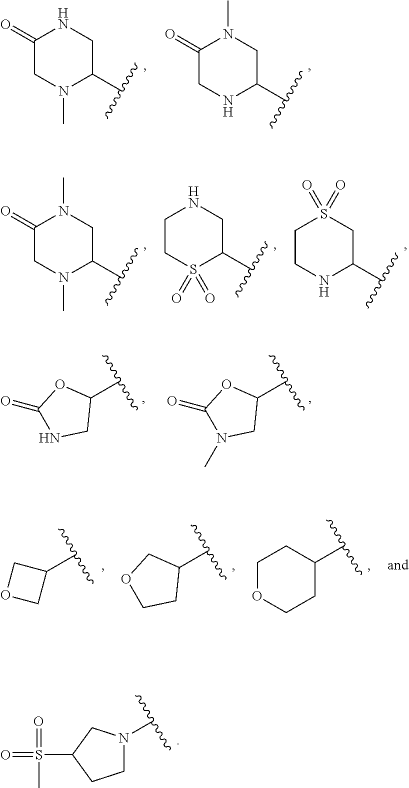

In some embodiments of Formula (II) or Formula (II-A), R⁴ is 3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more independently selected oxo substituents, or 6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, —C₁-C₆ alkyl, or —S(O)₂—(C₁-C₆ alkyl) substituents. In some embodiments, R⁴ is

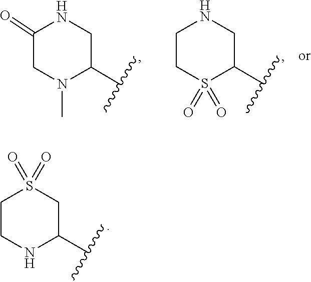

In some embodiments of Formula (II) or Formula (II-A), R⁴ is selected from the group consisting of:

5-membered heteroaryl comprising exactly two annular heteroatoms, one of which is a nitrogen atom and the other of which is an oxygen atom, wherein the 5-membered heteroaryl is substituted with exactly one methyl substituent,

5-membered heteroaryl comprising exactly two annular heteroatoms, both of which are nitrogen atoms, wherein the 5-membered heteroaryl is substituted with one or more methyl substituents, and

6-membered heteroaryl comprising one or two annular heteroatoms and optionally substituted with one or more methyl substituents, wherein the 6-membered heteroaryl is other than

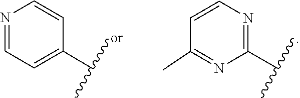

In some embodiments of Formula (II) or Formula (II-A), R⁴ is 5-membered heteroaryl comprising exactly two annular heteroatoms, one of which is a nitrogen atom and the other of which is an oxygen atom, wherein the 5-membered heteroaryl is substituted with exactly one methyl substituent. In other embodiments, R⁴ is 5-membered heteroaryl comprising exactly two annular heteroatoms, both of which are nitrogen atoms, wherein the 5-membered heteroaryl is substituted with one or more methyl substituents. In other embodiments, R⁴ is 6-membered heteroaryl comprising one or two annular heteroatoms and optionally substituted with one or more methyl substituents, wherein the 6-membered heteroaryl is other than

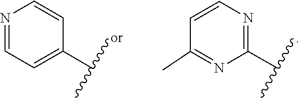

In some embodiments, R⁴ is selected from the group consisting of

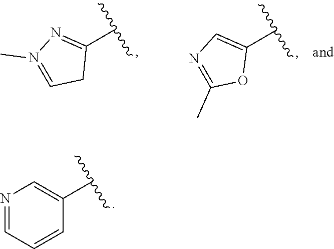

In some embodiments of Formula (II) or Formula (II-A), R⁴ is Z⁹—S(O)₂—, Z¹⁰—S(O)₂—NH—, Z¹¹—C(O)—NH—, Z¹²—CH₂—O—, Z¹³—O—, Z¹⁴—C(H)(C₁-C₆ alkyl)-NH—C(O)—,

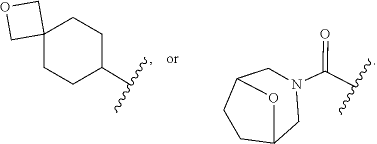 [SMILES: *C1CCC2(CC1)COC2 ; *C(=O)N1CC2CCC(C1)O2]

In some embodiments of Formula (II) or Formula (II-A), R⁴ is Z⁹—S(O)₂—. In some embodiments, the compound of Formula (II) or Formula (II-A) is a compound of Formula (II-A1)

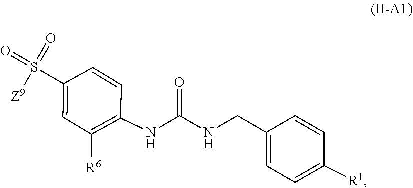

or a pharmaceutically acceptable salt thereof.

In some embodiments of Formula (II), Formula (II-A), or Formula (II-A1), Z⁹ is 3- to 10-membered heterocycloalkyl or hetercycloalkenyl optionally substituted with one or more independently selected R^{A} substituents, provided that Z⁹ is other than

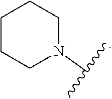 [SMILES: *N1CCCCC1]

In some embodiments, Z⁹ is 5- to 6-membered heterocycloalkyl or hetercycloalkenyl optionally substituted with one or more independently selected R^{A} substituents, provided that Z⁹ is other than

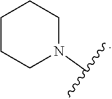 [SMILES: *N1CCCCC1]

In some embodiments, R^{A} is methyl or —CN. In some embodiments, Z⁹ is an unsubstituted 3- to 10-membered heterocycloalkyl or hetercycloalkenyl. In some embodiments, Z⁹ is an unsubstituted 5- to 6-membered heterocycloalkyl or hetercycloalkenyl.

In some embodiments, Z⁹ is C₁-C₆ alkyl optionally substituted with one or more independently selected R^{C} substituents, provided that Z⁹ is other than unsubstituted methyl or unsubstituted ethyl. In some embodiments, Z⁹ is C₁-C₃ alkyl optionally substituted with one or more independently selected R^{C} substituents, provided that Z⁹ is other than unsubstituted methyl or unsubstituted ethyl. In some embodiments, Z⁹ is unsubstituted C₃-C₆ alkyl. In some embodiments, Z⁹ is unsubstituted propyl. In some embodiments, Z⁹ is C₁-C₆ alkyl optionally substituted with one or more independently selected 3- to 8-membered heterocycloalkyl or heterocycloalkenyl. In some embodiments, Z⁹ is C₁-C₆ alkyl optionally substituted with one or more independently selected 5- to 6-membered heterocycloalkyl or heterocycloalkenyl.

In some embodiments, Z⁹ is —NH(C₁-C₆ alkyl). In some embodiments, Z⁹ is —NH(CH₃). In some embodiments, Z⁹ is —NH₂ substituted with one or more independently selected R^{B} substituents. In some embodiments, Z⁹ is —NH₂ substituted with one or more independently selected —C₁-C₆ alkyl-(5- to 10-membered heteroaryl). In some embodiments, Z⁹ is —NH₂ substituted with one or more independently selected —C₁-C₆ alkyl-(5- to 6-membered heteroaryl). In some embodiments, Z⁹ is —NH₂ substituted with one or more independently selected —C₁-C₆ alkyl-pyridinyl. In other embodiments, Z⁹ is 5- to 10-membered heteroaryl optionally substituted with one or more independently selected C₆-C₁₂ aryl. In other embodiments, Z⁹ is 5- to 6-membered heteroaryl optionally substituted with one or more phenyl.

In some embodiments, Z⁹ is cyclopropyl. In some embodiments, Z⁹ is C₆-C₁₂ aryl. In some embodiments, Z⁹ is phenyl.

In some embodiments, Z⁹ is selected from the group consisting of

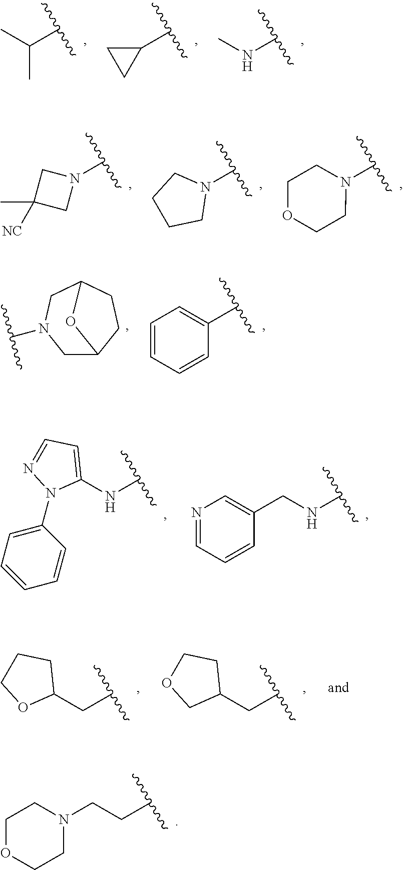

In some embodiments of Formula (II) or Formula (II-A), R⁴ is Z¹⁰—S(O)₂—NH—. In some embodiments, Z¹⁰ is C₁-C₆ alkyl substituted with one or more phenyl substituents. In some embodiments, Z¹⁰ is

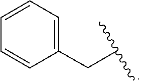 [SMILES: *Cc1ccccc1]

In some embodiments of Formula (II) or Formula (II-A), R⁴ is Z¹¹—C(O)—NH—. In some embodiments, Z¹¹ is C₃-C₁₀ cycloalkyl, provided that, when Z¹¹ is cyclopropyl, then R¹ is other than methoxy. In some embodiments, Z¹¹ is C₁-C₆ alkyl substituted with one or more independently selected 3- to 10-membered heterocycloalkyl or hetercycloalkenyl substituents. In some embodiments, Z¹¹ is C₁-C₆ alkyl substituted with one or more independently selected 5- to 6-membered heterocycloalkyl or hetercycloalkenyl substituents. In some embodiments, Z¹¹ is

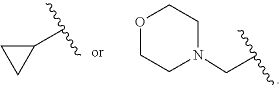

In some embodiments of Formula (II) or Formula (II-A), R⁴ is Z¹²—CH₂—O—. In some embodiments, Z¹² is selected from the group consisting of C₆-C₁₂ aryl, 5- to 10-membered heteroaryl, 3- to 10-membered heterocycloalkyl or heterocycloalkenyl, C₁-C₆ alkyl substituted with one or more independently selected 3- to 10-membered heterocycloalkyl or hetercycloalkenyl substituents or 5- to 10-membered heteroaryl substituents, and —C(O)-(3- to 10-membered heterocycloalkyl or heterocycloalkenyl). In some embodiments, Z¹² is C₆-C₁₂ aryl. In some embodiments, Z¹² is 5- to 10-membered heteroaryl. In some embodiments, Z¹² is 5- to 6-membered heteroaryl. In some embodiments, Z¹² is 3- to 10-membered heterocycloalkyl or heterocycloalkenyl. In other embodiments, Z¹² is 5- to 6-membered heterocycloalkyl or heterocycloalkenyl. In some embodiments, Z¹² is C₁-C₆ alkyl substituted with one or more independently selected 3- to 10-membered heterocycloalkyl or hetercycloalkenyl substituents or 5- to 10-membered heteroaryl substituents. In some embodiments, Z¹² is C₁-C₆ alkyl substituted with one or more independently selected 5- to 6-membered heterocycloalkyl or hetercycloalkenyl substituents or 5- to 6-membered heteroaryl substituents. In some embodiments, Z¹² is —C(O)-(3- to 10-membered heterocycloalkyl or heterocycloalkenyl). In other embodiments, Z¹² is —C(O)-(5- to 6-membered heterocycloalkyl or heterocycloalkenyl). In some embodiments, Z¹² is selected from the group consisting of

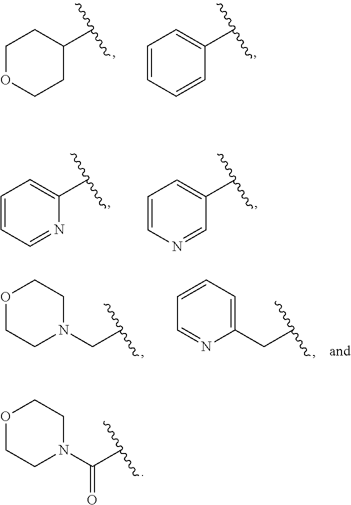

In some embodiments of Formula (II) or Formula (II-A), R⁴ is Z¹³—O—. In some embodiments, Z¹³ is 5- to 6-membered heteroaryl substituted with one or more independently selected —C(O)—NH(C₁-C₆ alkyl) substituents. In some embodiments, Z¹³ is pyridinyl substituted with one or more independently selected —C(O)—NH(C₁-C₆ alkyl) substituents. In some embodiments, Z¹³ is

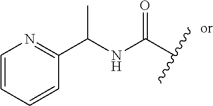 [SMILES: *C(=O)NC(C)c1ccccn1]

In some embodiments of Formula (II) or Formula (II-A), R⁴ is Z¹⁴—C(H)(C₁-C₆ alkyl)-NH—C(O)—. In some embodiments, R⁴ is Z¹⁴—C(H)(CH₃)—NH—C(O)—. In some embodiments, Z¹⁴ is 5- to 6-membered heteroaryl optionally substituted with one or more independently selected C₁-C₆ alkyl substituents. In some embodiments, Z¹⁴ is pyridinyl optionally substituted with one or more independently selected C₁-C₆ alkyl substituents. In some embodiments of Formula (II) or Formula (II-A), R⁴ is

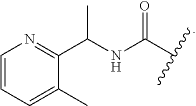 [SMILES: *C(=O)NC(C)c1ncccc1C]

In some embodiments of Formula (II) or Formula (II-A), R⁴ is

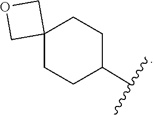 [SMILES: *C1CCC2(CC1)COC2]

In other embodiments, R⁴ is

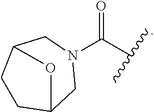 [SMILES: *C(=O)N1CC2CCC(C1)O2]

In some embodiments of Formula (II), or any variation thereof, including Formula (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1), R¹ is halo. For example, in some embodiments, R¹ is fluoro. In some embodiments, R¹ is chloro. In some embodiments, R¹ is bromo. In other embodiments, R¹ is iodo. In some embodiments, R¹ is methoxy. In some embodiments of Formula (II), or any variation thereof, including Formula (I-G), (I) (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), and (I-F), R² is hydrogen. In some embodiments, R² is C₁-C₆ alkyl. For example, in some embodiments, R² is methyl, ethyl, propyl, isopropyl, n-butyl, sec-butyl, or tert-butyl. In some embodiments of Formula (II), or any variation thereof, including Formula (I-G), (I) (I-A), (I-A1), (I-A2), (I-A3), (I-A4), (I-B), (I-B1), (I-B2), (I-B3), (I-C), (I-C1), (I-C2), (I-C3), (I-C4), (I-D), (I-D1), (I-D2), (I-D3), (I-D4), (I-D5), (I-D6), (I-D7), (I-E), and (I-F), R³ is hydrogen. In some embodiments, R³ is C₁-C₆ alkyl. For example, in some embodiments, R³ is methyl, ethyl, propyl, isopropyl, n-butyl, sec-butyl, or tert-butyl.

In some embodiments of Formula (II), or any variation thereof, including Formula (I-G), (I) (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), and (I-F), R² and R³ are each hydrogen. In some embodiments, R² is C₁-C₆ alkyl and R³ is hydrogen. For example, in some embodiments, R² is methyl, ethyl, propyl, isopropyl, n-butyl, sec-butyl, or tert-butyl, and R³ is hydrogen. In certain embodiments, R² is methyl and R³ is hydrogen. In some embodiments, R² is hydrogen and R³ is C₁-C₆ alkyl. For example, in some embodiments, R² is hydrogen, and R³ is methyl, ethyl, propyl, isopropyl, n-butyl, sec-butyl, or tert-butyl. In certain embodiments, R² is hydrogen and R³ is methyl.

In some embodiments, provided herein are compounds and salts thereof described in Table 1.

TABLE 1

| Compound | | |
|---|---|---|
| No. | Structure | Name |
| 1 | 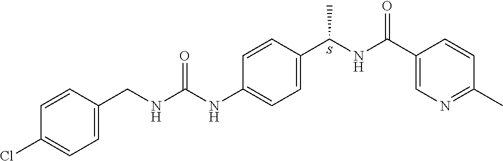 | [(4-{(1S)-1-[(6-methyl(3- pyridyl))carbonylamino]ethyl} phenyl)amino]-N-[(4- chlorophenyl)methyl]carboxamide |
| 2 | 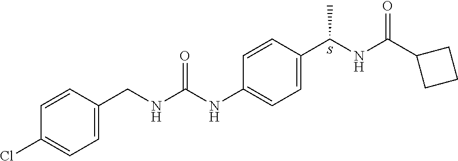 | ({4-[(1S)-1- (cyclobutylcarbonylamino)ethyl] phenyl}amino)-N-[(4- chlorophenyl)methyl]carboxamide |
| 3 | 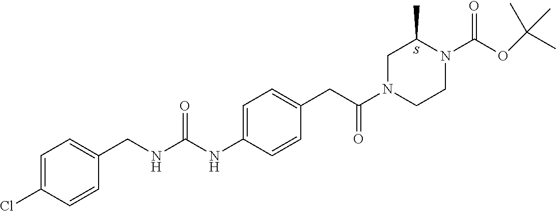 | tert-butyl (2R)-4-{2-[4-({[(4- chlorophenyl)methyl]amino}car bonylamino)phenyl]acetyl}-2- methylpiperazinecarboxylate |
| 4 | 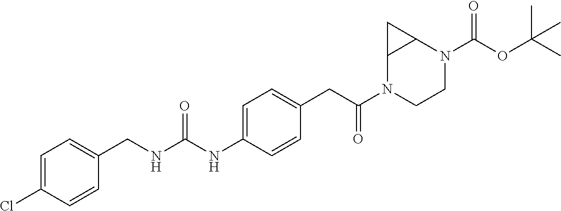 [SMILES: CC(C)(C)OC(=O)N1CCN(C(=O)Cc2ccc(NC(=O)NCc3ccc(Cl)cc3)cc2)C2CC21] | tert-butyl 5-(2-[4-({[(4- chlorophenyl)methyl]amino} carbonylamino)phenyl]acetyl}-2,5- diazabicyclo[4.1.0]heptane-2- carboxylate |
| 5 | 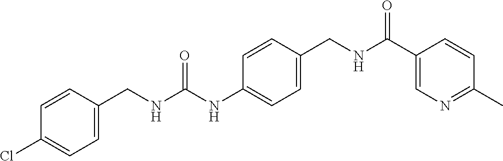 [SMILES: Cc1ccc(C(=O)NCc2ccc(NC(=O)NCc3ccc(Cl)cc3)cc2)cn1] | {[(4- chlorophenyl)methyl]amino}- N-(4-{[(6-methyl(3- pyridyl))carbonylamino]methyl} phenyl)carboxamide |
| 6 | 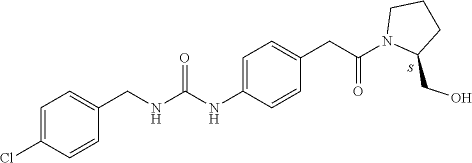 | N-(4-{2-[(2S)-2- (hydroxymethyl)pyrrolidinyl]- 2-oxoethyl}phenyl){[(4- chlorophenyl)methyl]amino} carboxamide |
| 7 | 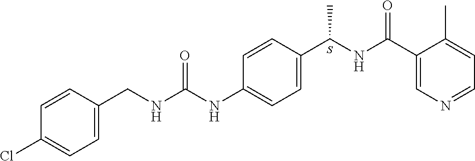 | [(4-{(1S)-1-[(4-methyl(3- pyridyl))carbonylamino]ethyl} phenyl)amino]-N-[(4- chlorophenyl)methyl]carboxamide |
| 8 | 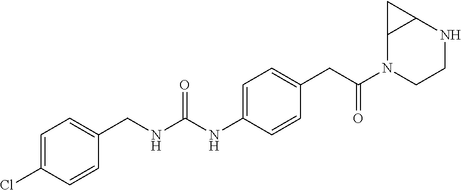 [SMILES: O=C(NCc1ccc(Cl)cc1)Nc1ccc(CC(=O)N2CCNC3CC32)cc1] | N-{4-[2-(2,5- diazabicyclo[4.1.0]hept-2-yl)-2- oxoethyl]phenyl}{[(4- chlorophenyl)methyl]amino} carboxamide |
| 9 | 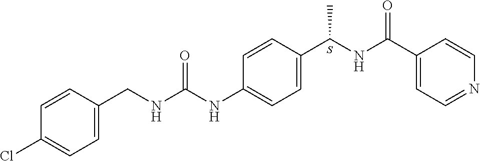 | ({4-[(1S)-1-(4- pyridylcarbonylamino)ethyl] phenyl}amino)-N-[(4- chlorophenyl)methyl]carboxamide |
| 10 | 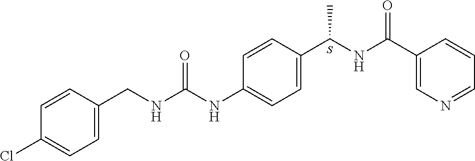 | ({4-[(1S)-1-(3- pyridylcarbonylamino)ethyl] phenyl}amino)-N-[(4- chlorophenyl)methyl]carboxamide |
| 11 | 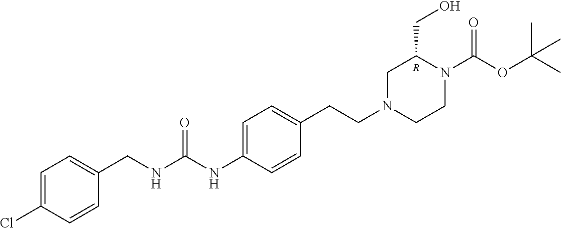 | tert-butyl (2R)-4-{2-[4-({[(4- chlorophenyl)methyl]amino} carbonylamino)phenyl]acetyl}-2- (hydroxymethyl) piperazinecarboxylate |
| 12 | 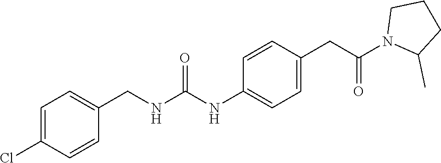 [SMILES: CC1CCCN1C(=O)Cc1ccc(NC(=O)NCc2ccc(Cl)cc2)cc1] | {[(4- chlorophenyl)methyl]amino}- N-{4-[2-(2-methylpyrrolidinyl)- 2- oxoethyl]phenyl}carboxamide |
| 13 | 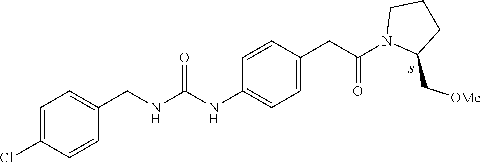 | N-(4-{2-[(2S)-2- (methoxymethyl)pyrrolidinyl]- 2-oxoethyl}phenyl){[(4- chlorophenyl)methyl]amino} carboxamide |
| 14 | 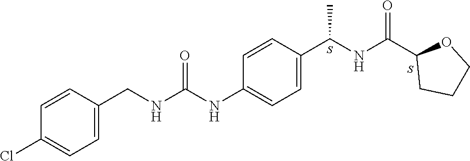 | [(4-{(1S)-1-[((2S)oxolan-2- yl)carbonylamino]ethyl}phenyl) amino]-N-[(4- chlorophenyl)methyl]carboxamide |
| 15 | 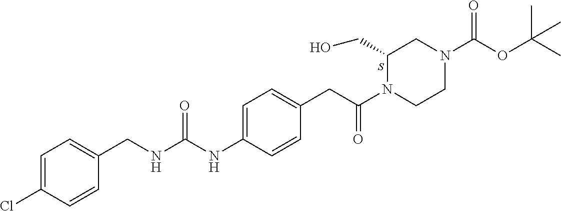 | tert-butyl (3S)-4-{2-[4-({[(4- chlorophenyl)methyl]amino} carbonylamino)phenyl]acetyl}-3- (hydroxymethyl) piperazinecarboxylate |
| 16 | 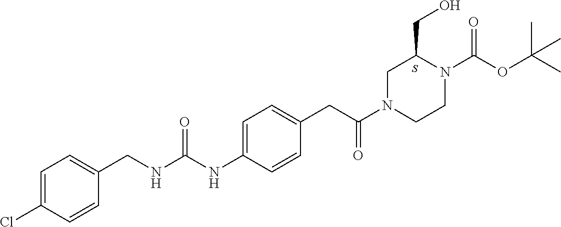 | tert-butyl (2S)-4-{2-[4-({[(4- chlorophenyl)methyl]amino} carbonylamino)phenyl]acetyl}-2- (hydroxymethyl) piperazinecarboxylate |
| 17 | 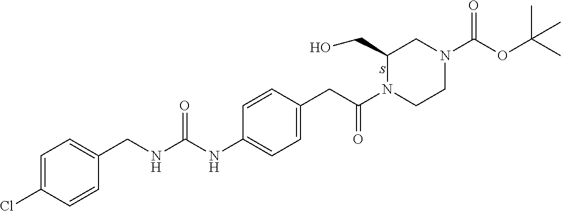 | tert-butyl (3R)-4-{2-[4-({[(4- chlorophenyl)methyl]amino} carbonylamino)phenyl]acetyl}-3- (hydroxymethyl) piperazinecarboxylate |
| 18 | 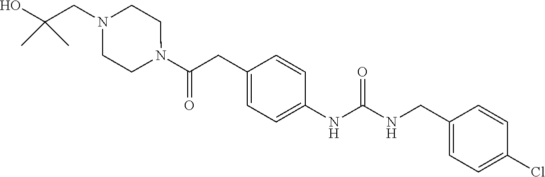 [SMILES: CC(C)(O)CN1CCN(C(=O)Cc2ccc(NC(=O)NCc3ccc(Cl)cc3)cc2)CC1] | {[(4- chlorophenyl)methyl]amino}- N-(4-{2-[4-(2-hydroxy-2- methylpropyl)piperazinyl]-2- oxoethyl}phenyl)carboxamide |
| 19 | 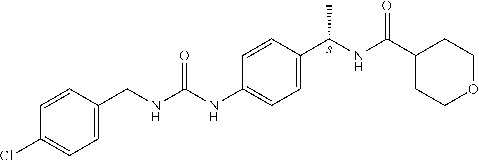 | ({4-[(1S)-1-(2H-3,4,5,6- tetrahydropyran-4- ylcarbonylamino)ethyl]phenyl} amino)-N-[(4- chlorophenyl)methyl] carboxamide |
| 20 | 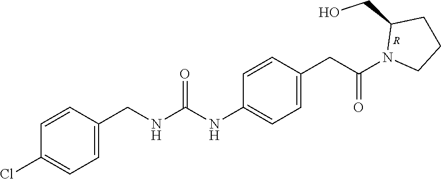 | [(4-{2-[(2R)-2- (hydroxymethyl)pyrrolidinyl]- 2-oxoethyl}phenyl)amino]-N- [(4-chlorophenyl)methyl] carboxamide |
| 21 | 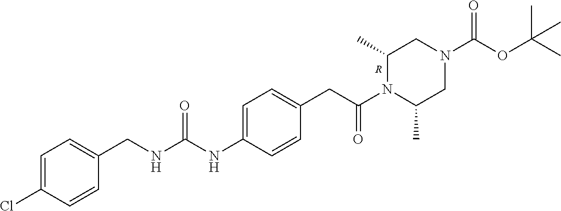 | tert-butyl (3S,5R)-4-{2-[4- ({[(4- chlorophenyl)methyl]amino}car bonylamino)phenyl]acetyl}-3,5- dimethylpiperazinecarboxylate |
| 22 | 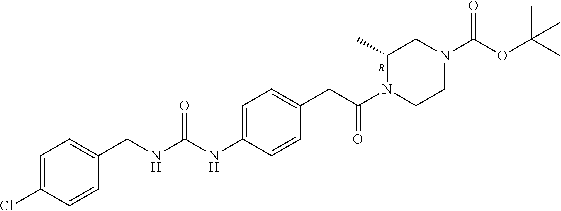 | tert-butyl (3R)-4-{2-[4-({[(4- chlorophenyl)methyl]amino} carbonylamino)phenyl]acetyl}-3- methylpiperazinecarboxylate |
| 23 | 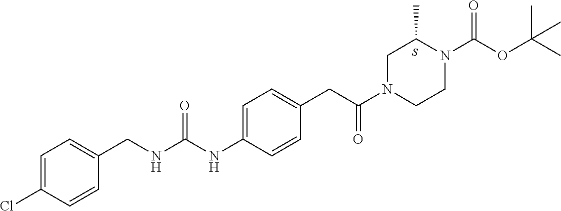 | tert-butyl (2S)-4-{2-[4-({[(4- chlorophenyl)methyl]amino} carbonylamino)phenyl]acetyl}-2- methylpiperazinecarboxylate |
| 24 | 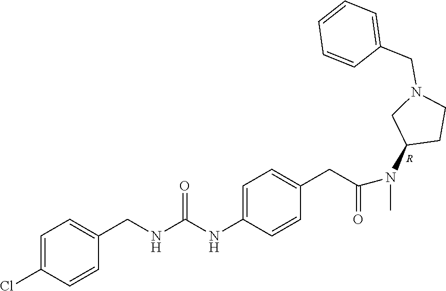 | N-[(3R)-1-benzylpyrrolidin-3- yl]-2-[4-({[(4- chlorophenyl)methyl]amino} carbonylamino)phenyl]-N- methylacetamide |
| 25 | 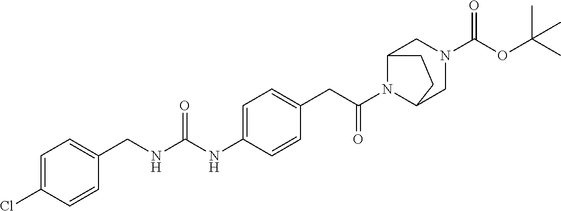 [SMILES: CC(C)(C)OC(=O)N1CC2CCC(C1)N2C(=O)Cc1ccc(NC(=O)NCc2ccc(Cl)cc2)cc1] | tert-butyl 8-{2-[4-({[(4- chlorophenyl)methyl]amino} carbonylamino)phenyl]acetyl}-3,8- diazabicyclo[3.2.1]octane-3- carboxylate |
| 26 | 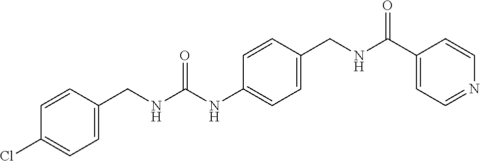 [SMILES: O=C(NCc1ccc(Cl)cc1)Nc1ccc(CNC(=O)c2ccncc2)cc1] | {[(4- chlorophenyl)methyl]amino}- N-{4-[(4- pyridylcarbonylamino)methyl] phenyl}carboxamide |
| 27 | 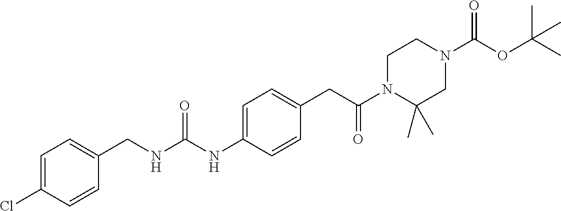 [SMILES: CC(C)(C)OC(=O)N1CCN(C(=O)Cc2ccc(NC(=O)NCc3ccc(Cl)cc3)cc2)C(C)(C)C1] | tert-butyl 4-{2-[4-({[(4- chlorophenyl)methyl]amino} carbonylamino)phenyl]acetyl}-3,3- dimethylpiperazinecarboxylate |
| 28 | 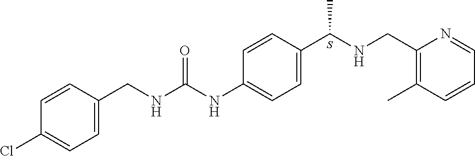 | {[4-((1S)-1-{[(3-methyl(2- pyridyl))methyl]amino}ethyl) phenyl]amino}-N-[(4- chlorophenyl)methyl]carboxamide |
| 29 | 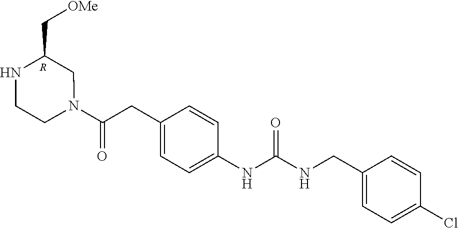 | [(4-{2-[(3R)-3- (methoxymethyl)piperazinyl]-2- oxoethyl}phenyl)amino]-N-[(4- chlorophenyl)methyl]carboxamide |
| 30 | 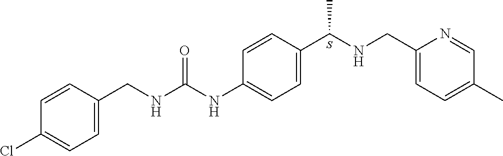 | {[4-((1S)-1-{[(5-methyl(2- pyridyl))methyl]amino}ethyl) phenyl]amino}-N-[(4- chlorophenyl)methyl]carboxamide |
| 31 | 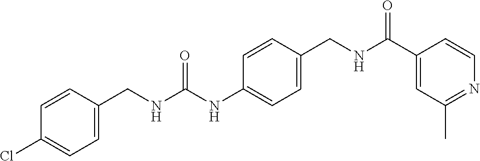 [SMILES: Cc1cc(C(=O)NCc2ccc(NC(=O)NCc3ccc(Cl)cc3)cc2)ccn1] | {[(4- chlorophenyl)methyl]amino}- N-(4-{[(2-methyl(4- pyridyl))carbonylamino]methyl} phenyl)carboxamide |
| 32 | 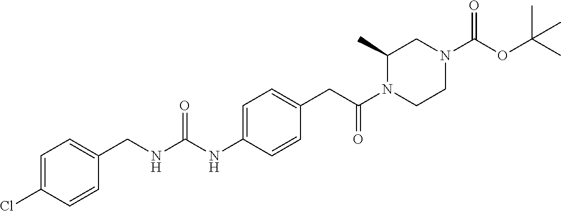 [SMILES: C[C@H]1CN(C(=O)OC(C)(C)C)CCN1C(=O)Cc1ccc(NC(=O)NCc2ccc(Cl)cc2)cc1] | tert-butyl (3S)-4-{2-[4-({[(4- chlorophenyl)methyl]amino} carbonylamino)phenyl]acetyl}-3- methylpiperazinecarboxylate |
| 33 | 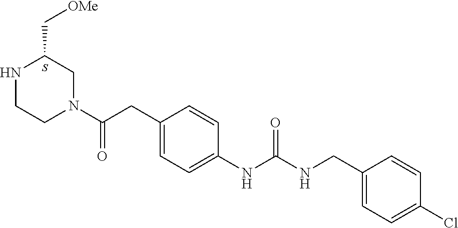 | [(4-{2-[(3S)-3- (methoxymethyl)piperazinyl]-2- oxoethyl}phenyl)amino]-N-[(4- chlorophenyl)methyl] carboxamide |
| 34 | 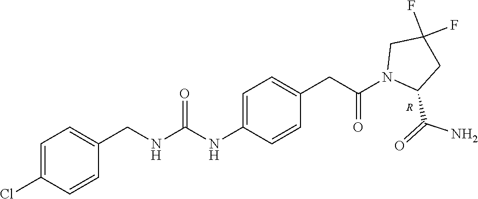 | (2R)-1-{2-[4-({[(4- chlorophenyl)methyl]amino}car bonylamino)phenyl]acetyl}-4,4- difluoropyrrolidine-2- carboxamide |
| 35 | 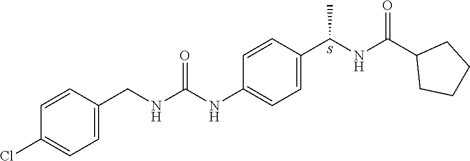 | ({4-[(1S)-1- (cyclopentylcarbonylamino) ethyl]phenyl}amino)-N-[(4- chlorophenyl)methyl] carboxamide |
| 36 | 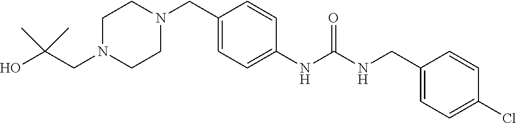 [SMILES: CC(C)(O)CN1CCN(Cc2ccc(NC(=O)NCc3ccc(Cl)cc3)cc2)CC1] | {[(4- chlorophenyl)methyl]amino}- N-(4-{[4-(2-hydroxy-2- methylpropyl)piperazinyl] methyl}phenyl)carboxamide |
| 37 | 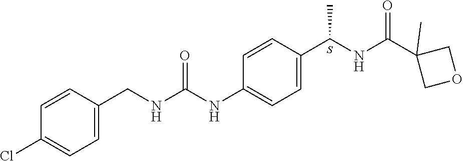 | [(4-{(1S)-1-[(3-methyloxetan- 3-yl)carbonylamino]ethyl} phenyl)amino]-N-[(4- chlorophenyl)methyl] carboxamide |
| 38 | 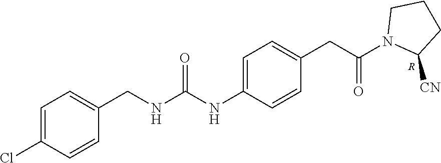 | N-{4-[2-((2S)-2- cyanopyrrolidinyl)-2- oxoethyl]phenyl}{[(4- chlorophenyl)methyl]amino} carboxamide |
| 39 | 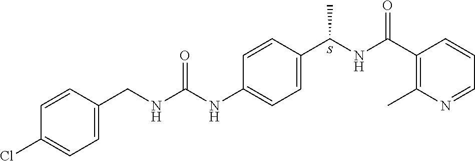 | [(4-{(1S)-1-[(2-methyl(3- pyridyl))carbonylamino]ethyl} phenyl)amino]-N-[(4- chlorophenyl)methyl] carboxamide |
| 40 | 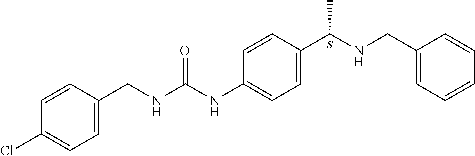 | [(4-{(1S)-1- [benzylamino]ethyl}phenyl) amino]-N-[(4- chlorophenyl)methyl]carboxamide |
| 41 | 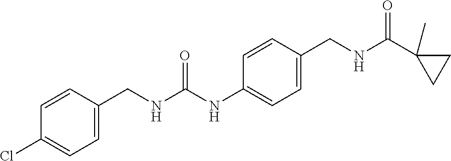 [SMILES: CC1(C(=O)NCc2ccc(NC(=O)NCc3ccc(Cl)cc3)cc2)CC1] | {[(4- chlorophenyl)methyl]amino}- N-(4-{[(methylcyclopropyl) carbonylamino]methyl}phenyl) carboxamide |
| 42 | 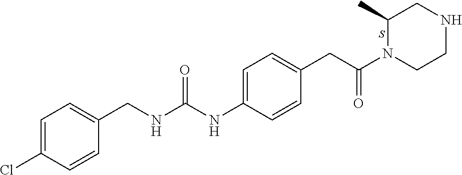 | N-{4-[2-((2S)-2- methylpiperazinyl)-2- oxoethyl]phenyl}{[(4- chlorophenyl)methyl]amino} carboxamide |
| 43 | 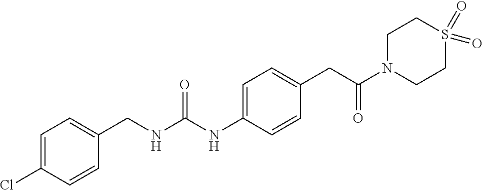 [SMILES: O=C(NCc1ccc(Cl)cc1)Nc1ccc(CC(=O)N2CCS(=O)(=O)CC2)cc1] | N-{4-[2-(1,1-dioxo(1,4- thiazaperhydroin-4-yl))-2- oxoethyl]phenyl}{[(4- chlorophenyl)methyl]amino} carboxamide |
| 44 | 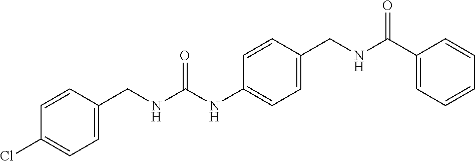 [SMILES: O=C(NCc1ccc(Cl)cc1)Nc1ccc(CNC(=O)c2ccccc2)cc1] | {[(4- chlorophenyl)methyl]amino}- N-{4- [(phenylcarbonylamino)methyl] phenyl}carboxamide |
| 45 | 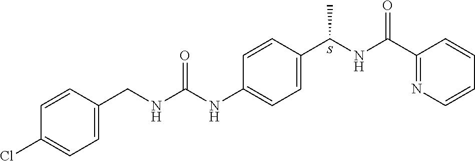 | ({4-[(1S)-1-(2- pyridylcarbonylamino)ethyl] phenyl}amino)-N-[(4- chlorophenyl)methyl] carboxamide |
| 46 | 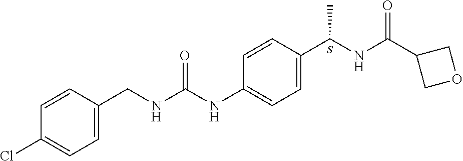 | ({4-[(1S)-1-(oxetan-3- ylcarbonylamino)ethyl]phenyl} amino)-N-[(4- chlorophenyl)methyl]carboxamide |
| 47 | 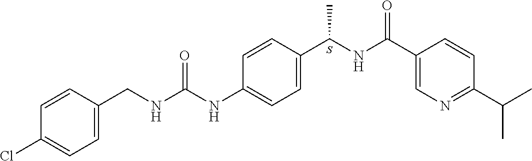 | {[(4- chlorophenyl)methyl]amino}- N-[4-({[6-(methylethyl)(3- pyridyl)]carbonylamino}methyl) phenyl]carboxamide |
| 48 | 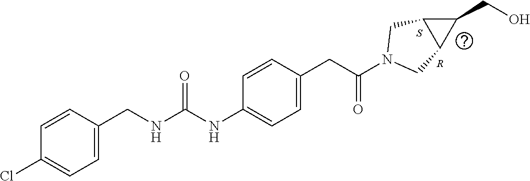 | [(4-{2-[(5S,1R)-6- (hydroxymethyl)-3- azabicyclo[3.1.0]hex-3-yl]-2- oxoethyl}phenyl)amino]-N-[(4- chlorophenyl)methyl]carboxamide |
| 49 | 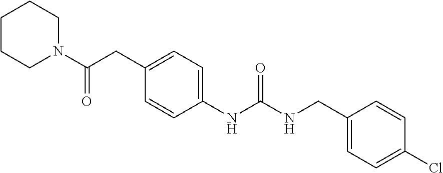 [SMILES: O=C(NCc1ccc(Cl)cc1)Nc1ccc(CC(=O)N2CCCCC2)cc1] | N-[(4-chlorophenyl)methyl]{[4- (2-oxo-2- piperidylethyl)phenyl]amino} carboxamide |
| 50 | 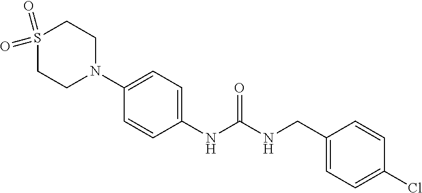 [SMILES: O=C(NCc1ccc(Cl)cc1)Nc1ccc(N2CCS(=O)(=O)CC2)cc1] | {[4-(1,1-dioxothian-4- yl)phenyl]amino}-N-[(4- chlorophenyl)methyl]carboxamide |
| 51 | 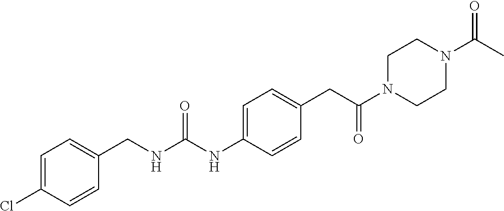 [SMILES: CC(=O)N1CCN(C(=O)Cc2ccc(NC(=O)NCc3ccc(Cl)cc3)cc2)CC1] | N-{4-[2-(4-acetylpiperazinyl)- 2-oxoethyl]phenyl}{[(4- chlorophenyl)methyl]amino} carboxamide |
| 52 | 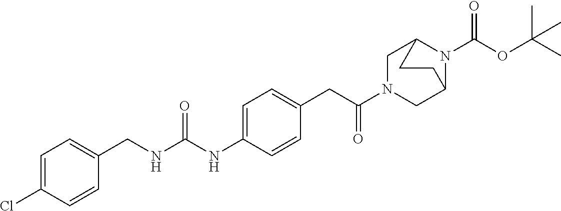 [SMILES: CC(C)(C)OC(=O)N1C2CCC1CN(C(=O)Cc1ccc(NC(=O)NCc3ccc(Cl)cc3)cc1)C2] | tert-butyl 3-{2-[4-({[(4- chlorophenyl)methyl]amino}car bonylamino)phenyl]acetyl}-3,8- diazabicyclo[3.2.1]octane-8- carboxylate |
| 53 | 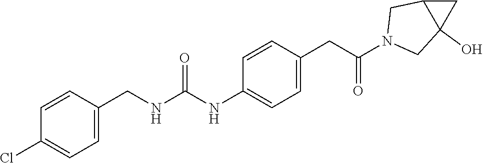 [SMILES: O=C(NCc1ccc(Cl)cc1)Nc1ccc(CC(=O)N2CC3CC3(O)C2)cc1] | N-[(4-chlorophenyl)methyl]({4- [2-(1-hydroxy-3- azabicyclo[3.1.0]hex-3-yl)-2- oxoethyl]phenyl}amino) carboxamide |
| 54 | 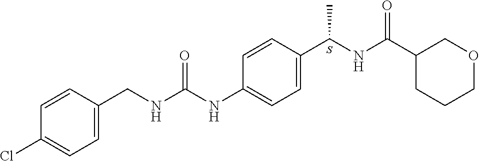 | ({4-[(1S)-1-(2H-3,4,5,6- tetrahydropyran-3- ylcarbonylamino)ethyl]phenyl} amino)-N-[(4- chlorophenyl)methyl]carboxamide |
| 55 | 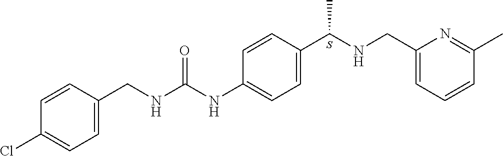 | {[4-((1S)-1-{[(6-methyl(2- pyridyl))methyl]amino}ethyl) phenyl]amino}-N-[(4- chlorophenyl)methyl] carboxamide |
| 56 | 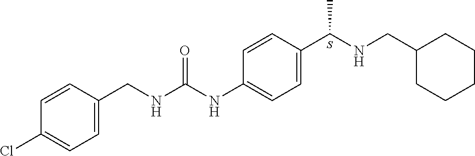 | [(4-((S)-1- [(cyclohexylmethyl)amino] ethyl}phenyl)amino]-N-[(4- chlorophenyl)methyl]carboxamide |
| 57 | 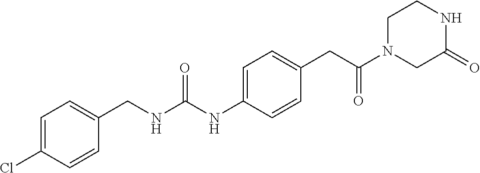 [SMILES: O=C1CN(C(=O)Cc2ccc(NC(=O)NCc3ccc(Cl)cc3)cc2)CCN1] | {[(4- chlorophenyl)methyl]amino}- N-{4-[2-oxo-2-(3- oxopiperazinyl)ethyl]phenyl} carboxamide |
| 58 | 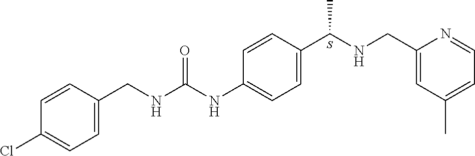 | {[4-((1S)-1-{[(4-methyl(2- pyridyl))methyl]amino}ethyl) phenyl]amino}-N-[(4- chlorophenyl)methyl]carboxamide |
| 59 | 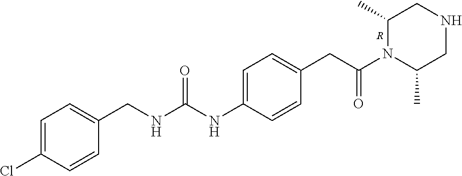 | N-{4-[2-((2S,6R)-2,6- dimethylpiperazinyl)-2- oxoethyl]phenyl]{[(4- chlorophenyl)methyl]amino} carboxamide |
| 60 | 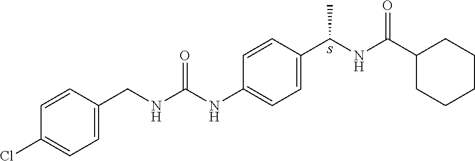 | ({4-[(1S)-1- (cyclohexylcarbonylamino) ethyl]phenyl}amino)-N-[(4- chlorophenyl)methyl]carboxamide |
| 61 | 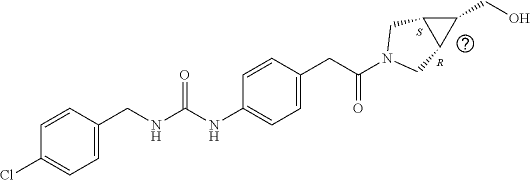 | [(4-{2-[(5S,1R)-6- (hydroxymethyl)-3- azabicyclo[3.1.0]hex-3-yl]-2- oxoethyl}phenyl)amino]-N-[(4- chlorophenyl)methyl]carboxamide |
| 62 | 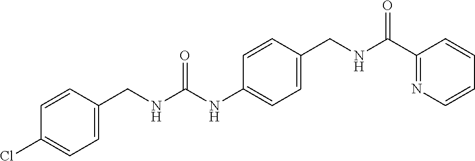 [SMILES: O=C(NCc1ccc(Cl)cc1)Nc1ccc(CNC(=O)c2ccccn2)cc1] | {[(4- chlorophenyl)methyl]amino}- N-{4-[(2- pyridylcarbonylamino)methyl] phenyl}carboxamide |
| 63 | 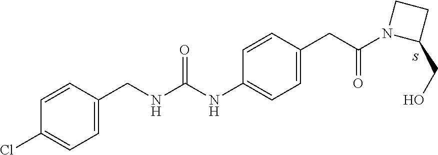 | N-(4-{2-[(2S)-2- (hydroxymethyl)azetidinyl]-2- oxoethyl}phenyl){[(4- chlorophenyl)methyl]amino} carboxamide |
| 64 | 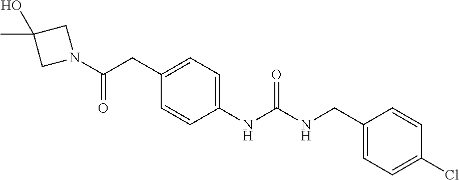 [SMILES: CC1(O)CN(C(=O)Cc2ccc(NC(=O)NCc3ccc(Cl)cc3)cc2)C1] | N-[(4-chlorophenyl)methyl]({4- [2-(3-hydroxy-3- methylazetidinyl)-2- oxoethyl]phenyl}amino) carboxamide |
| 65 | 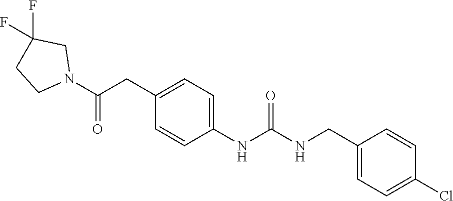 [SMILES: O=C(NCc1ccc(Cl)cc1)Nc1ccc(CC(=O)N2CCC(F)(F)C2)cc1] | ({4-[2-(3,3- difluoropyrrolidinyl)-2- oxoethyl]phenyl}amino)-N-[(4- chlorophenyl)methyl]carboxamide |
| 66 | 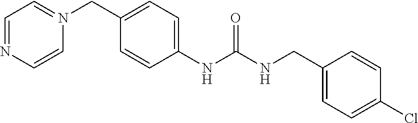 | N-[(4-chlorophenyl)methyl]{[4- (4-pyridylmethyl)phenyl]amino} carboxamide |
| 67 | 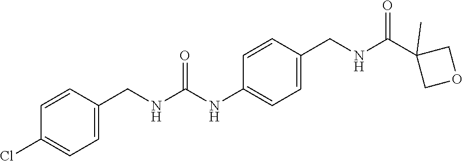 [SMILES: CC1(C(=O)NCc2ccc(NC(=O)NCc3ccc(Cl)cc3)cc2)COC1] | {[(4-chlorophenyl)methyl]amino}- N-(4-{[(3-methyloxetan-3- yl)carbonylamino]methyl} phenyl)carboxamide |
| 68 | 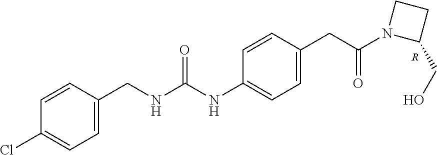 | N-(4-{2-[(2R)-2- (hydroxymethyl)azetidinyl]-2- oxoethyl}phenyl){[(4- chlorophenyl)methyl]amino} carboxamide |
| 69 | 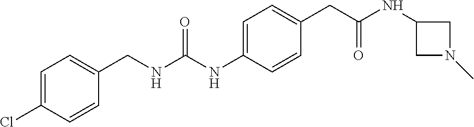 [SMILES: CN1CC(NC(=O)Cc2ccc(NC(=O)NCc3ccc(Cl)cc3)cc2)C1] | 2-[4-({[(4- chlorophenyl)methyl]amino} carbonylamino)phenyl]-N-(1- methylazetidin-3-yl)acetamide |
| 70 | 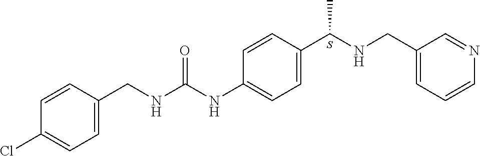 | [(4-{(1S)-1-[(3- pyridylmethyl)amino]ethyl} phenyl)amino]-N-[(4- chlorophenyl)methyl]carboxamide |
| 71 | 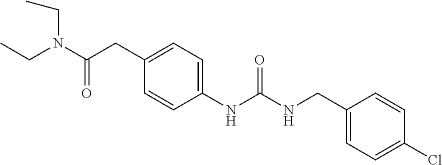 [SMILES: CCN(CC)C(=O)Cc1ccc(NC(=O)NCc2ccc(Cl)cc2)cc1] | 2-[4-({N-[(4- chlorophenyl)methyl]carbamoyl} amino)phenyl]-N,N- diethylacetamide |
| 72 | 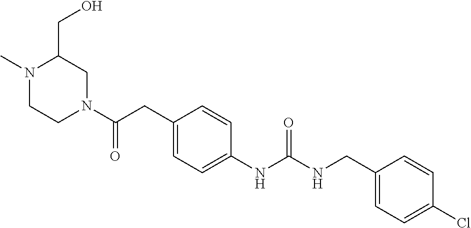 [SMILES: CN1CCN(C(=O)Cc2ccc(NC(=O)NCc3ccc(Cl)cc3)cc2)CC1CO] | N-[(4-chlorophenyl)methyl][(4- {2-[3-(hydroxymethyl)-4- methylpiperazinyl]-2- oxoethyl}phenyl)amino] carboxamide |
| 73 | 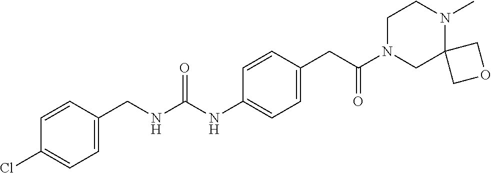 [SMILES: CN1CCN(C(=O)Cc2ccc(NC(=O)NCc3ccc(Cl)cc3)cc2)CC12COC2] | {[(4-chlorophenyl)methyl]amino}- N-{4-[2-(9-methyl-2-oxa-6,9- diazaspiro[3.5]non-6-yl)-2- oxoethyl]phenyl}carboxamide |
| 74 | 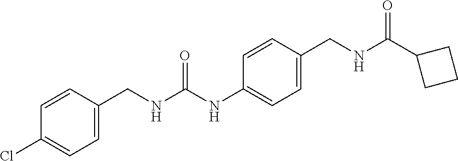 [SMILES: O=C(NCc1ccc(Cl)cc1)Nc1ccc(CNC(=O)C2CCC2)cc1] | {[(4-chlorophenyl)methyl]amino}- N-{4-[(cyclobutylcarbonylamino) methyl]phenyl}carboxamide |
| 75 | 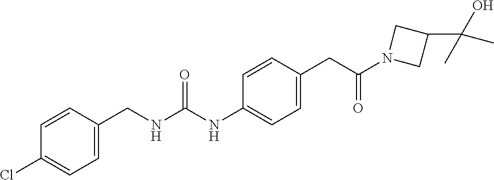 [SMILES: CC(C)(O)C1CN(C(=O)Cc2ccc(NC(=O)NCc3ccc(Cl)cc3)cc2)C1] | {[(4-chlorophenyl)methyl]amino}- N-(4-{2-[3-(1-hydroxy- isopropyl)azetidinyl]-2- oxoethyl}phenyl)carboxamide |
| 76 | 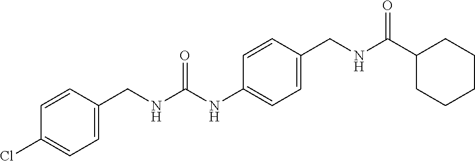 [SMILES: O=C(NCc1ccc(Cl)cc1)Nc1ccc(CNC(=O)C2CCCCC2)cc1] | {[(4-chlorophenyl)methyl]amino}- N-{4-[(cyclohexylcarbonylamino) methyl]phenyl}carboxamide |
| 77 | 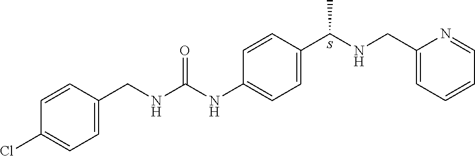 | [(4-{(1S)-1-[(2- pyridylmethyl)amino]ethyl} phenyl)amino]-N-[(4- chlorophenyl)methyl]carboxamide |
| 78 | 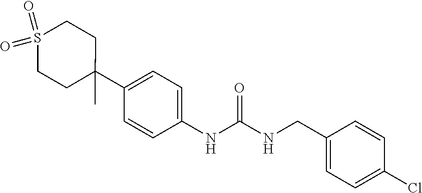 [SMILES: CC1(c2ccc(NC(=O)NCc3ccc(Cl)cc3)cc2)CCS(=O)(=O)CC1] | N-[(4-chlorophenyl)methyl]{[4- (4-methyl-1,1-dioxothian-4- yl)phenyl]amino}carboxamide |
| 79 | 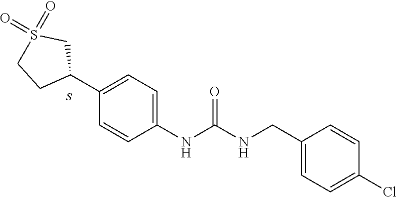 | {[4-((3S)-1,1-dioxothiolan-3- yl)phenyl]amino}-N-[(4- chlorophenyl)methyl]carboxamide |
| 80 | 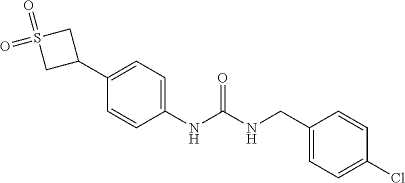 [SMILES: O=C(NCc1ccc(Cl)cc1)Nc1ccc(C2CS(=O)(=O)C2)cc1] | {[4-(1,1-dioxothietan-3- yl)phenyl]amino}-N-[(4- chlorophenyl)methyl]carboxamide |
| 81 | 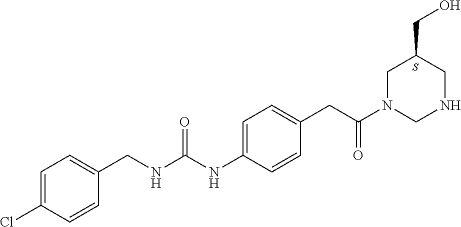 | N-(4-{2-[(3S)-3- (hydroxymethyl)piperazinyl]-2- oxoethyl}phenyl){[(4- chlorophenyl)methyl]amino} carboxamide |
| 82 | 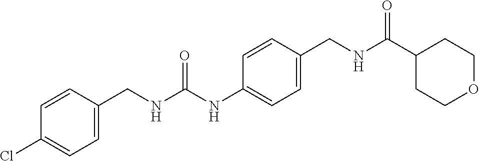 [SMILES: O=C(NCc1ccc(Cl)cc1)Nc1ccc(CNC(=O)C2CCOCC2)cc1] | {[(4- chlorophenyl)methyl]amino}- N-{4-[(2H-3,4,5,6- tetrahydropyran-4- ylcarbonylamino)methyl]phenyl} carboxamide |
| 83 | 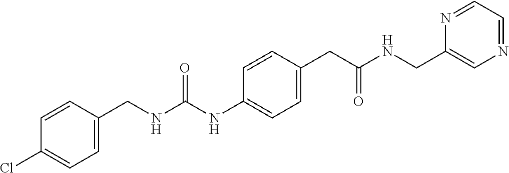 [SMILES: O=C(Cc1ccc(NC(=O)NCc2ccc(Cl)cc2)cc1)NCc1cnccn1] | 2-[4-({N-[(4- chlorophenyl)methyl]carbamoyl }amino)phenyl]-N-(pyrazin-2- ylmethyl)acetamide |
| 84 | 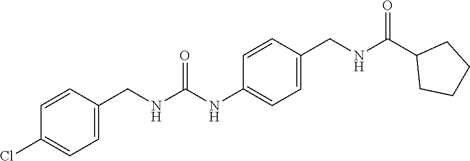 [SMILES: O=C(NCc1ccc(Cl)cc1)Nc1ccc(CNC(=O)C2CCCC2)cc1] | {[(4-chlorophenyl)methyl]amino}- N-{4-[(cyclopentylcarbonylamino) methyl]phenyl}carboxamide |
| 85 | 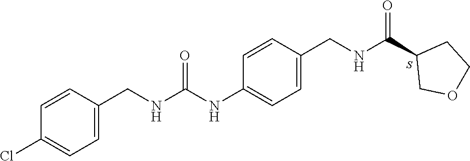 | N-(4-{[((3S)oxolan-3- yl)carbonylamino]methyl} phenyl){[(4- chlorophenyl)methyl]amino} carboxamide |
| 86 | 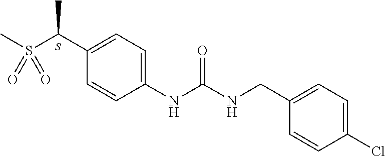 | N-{4-[(1S)-1- (methylsulfonyl)ethyl]phenyl} {[(4- chlorophenyl)methyl]amino} carboxamide |
| 87 | 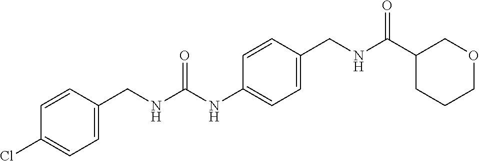 [SMILES: O=C(NCc1ccc(Cl)cc1)Nc1ccc(CNC(=O)C2CCCOC2)cc1] | {[(4-chlorophenyl)methyl]amino}- N-{4-[(2H-3,4,5,6- tetrahydropyran-3- ylcarbonylamino)methyl]phenyl} carboxamide |
| 88 | 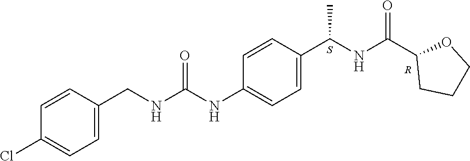 | [(4-{1-[((2R)oxolan-2- yl)carbonylamino](1S)ethyl} phenyl)amino]-N-[(4- chlorophenyl)methyl]carboxamide |
| 89 | 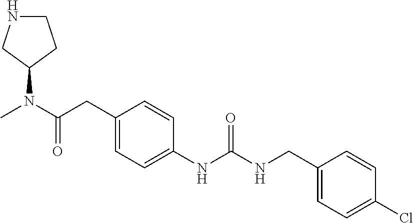 [SMILES: CN(C(=O)Cc1ccc(NC(=O)NCc2ccc(Cl)cc2)cc1)[C@@H]1CCNC1] | N-((3R)pyrrolidin-3-yl)-2-[4- ({N-[(4- chlorophenyl)methyl]carbamoyl} amino)phenyl]-N- methylacetamide |
| 90 | 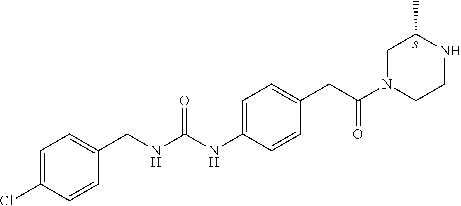 | N-{4-[2-((3S)-3- methylpiperazinyl)-2- oxoethyl]phenyl}{[(4- chlorophenyl)methyl]amino} carboxamide |
| 91 | 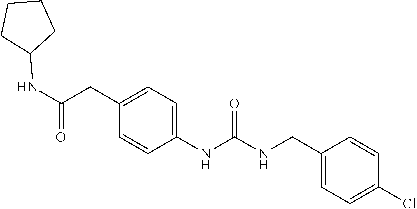 [SMILES: O=C(Cc1ccc(NC(=O)NCc2ccc(Cl)cc2)cc1)NC1CCCC1] | 2-[4-({N-[(4- chlorophenyl)methyl]carbamoyl} amino)phenyl]-N- cyclopentylacetamide |
| 92 | 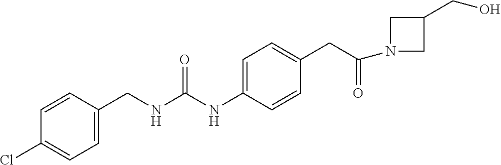 [SMILES: O=C(NCc1ccc(Cl)cc1)Nc1ccc(CC(=O)N2CC(CO)C2)cc1] | {[(4- chlorophenyl)methyl]amino}- N-(4-{2-[3- (hydroxymethyl)azetidinyl]-2- oxoethyl}phenyl)carboxamide |
| 93 | 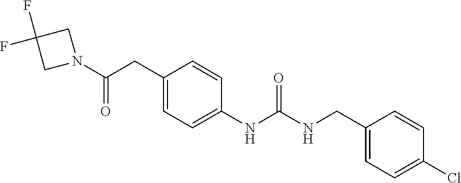 [SMILES: O=C(NCc1ccc(Cl)cc1)Nc1ccc(CC(=O)N2CC(F)(F)C2)cc1] | N-(4-[2-(3,3- difluoroazetidinyl)-2- oxoethyl]phenyl}{[(4- chlorophenyl)methyl]amino} carboxamide |
| 94 | 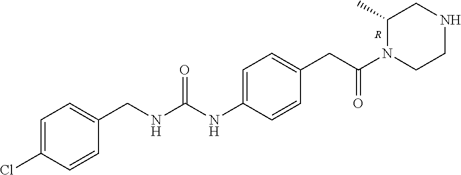 | N-{4-[2-((2R)-2- methylpiperazinyl)-2- oxoethyl]phenyl}{[(4- chlorophenyl)methyl]amino} carboxamide |
| 95 | 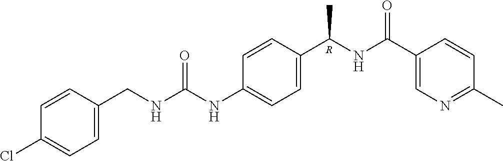 | [(4-{(1R)-1-[(6-methyl(3- pyridyl))carbonylamino]ethyl} phenyl)amino]-N-[(4- chlorophenyl)methyl]carboxamide |
| 96 | 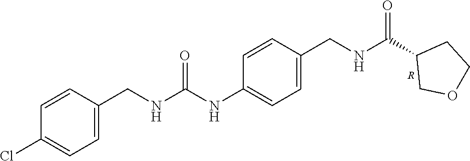 | N-(4-{[((3R)oxolan-3- yl)carbonylamino]methyl} phenyl){[(4- chlorophenyl)methyl]amino} carboxamide |
| 97 | 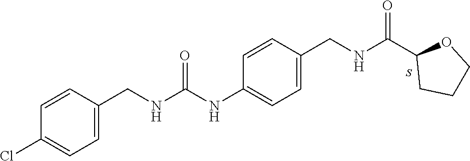 | N-(4-{[((2S)oxolan-2- yl)carbonylamino]methyl} phenyl){[(4- chlorophenyl)methyl]amino} carboxamide |
| 98 | 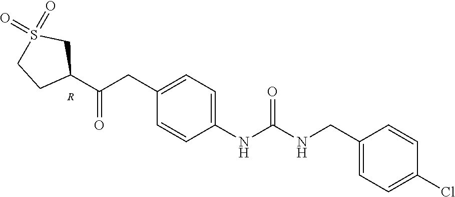 | {[4-((3R)-1,1-dioxothiolan-3- yl)phenyl]amino}-N-[(4- chlorophenyl)methyl]carboxamide |
| 99 | 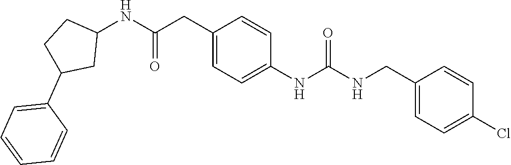 [SMILES: O=C(Cc1ccc(NC(=O)NCc2ccc(Cl)cc2)cc1)NC1CCC(c2ccccc2)C1] | 2-[4-({[(4- chlorophenyl)methyl]amino} carbonylamino)phenyl]-N-(3- phenylcyclopentyl)acetamide |
| 100 | 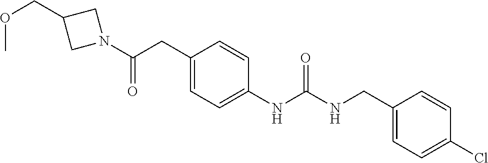 [SMILES: COCC1CN(C(=O)Cc2ccc(NC(=O)NCc3ccc(Cl)cc3)cc2)C1] | {[(4- chlorophenyl)methyl]amino}- N-(4-{2-[3- (methoxymethyl)azetidinyl]-2- oxoethyl}phenyl)carboxamide |
| 101 | 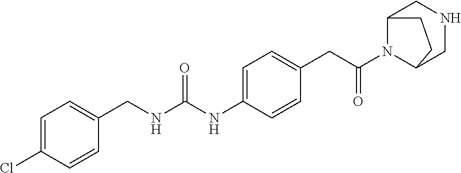 [SMILES: O=C(NCc1ccc(Cl)cc1)Nc1ccc(CC(=O)N2C3CCC2CNC3)cc1] | N-{4-[2-(3,8- diazabicyclo[3.2.1]oct-8-yl)-2- oxoethyl]phenyl}{[(4- chlorophenyl)methyl]amino} carboxamide |
| 102 | 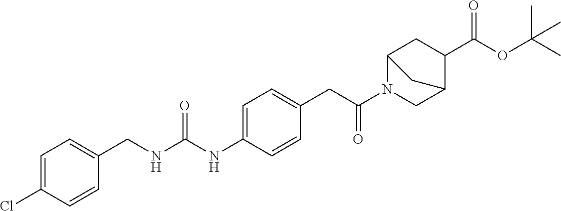 [SMILES: CC(C)(C)OC(=O)C1CC2CC1CN2C(=O)Cc1ccc(NC(=O)NCc2ccc(Cl)cc2)cc1] | tert-butyl 5-(2-[4-(I(4- chlorophenyl)methyl]amino} carbonylamino)phenyl]acetyl}-2,5- diazabicyclo[2.2.1]heptane-2- carboxylate |
| 103 | 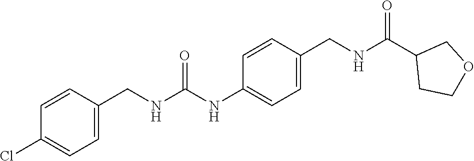 [SMILES: O=C(NCc1ccc(Cl)cc1)Nc1ccc(CNC(=O)C2CCOC2)cc1] | {[(4- chlorophenyl)methyl]amino}- N-{4-[(oxolan-3- ylcarbonylamino)methyl]phenyl} carboxamide |
| 104 | 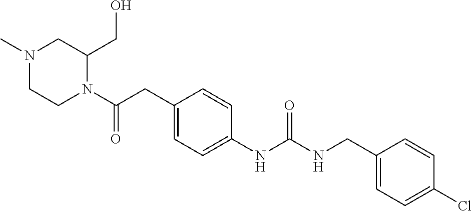 [SMILES: CN1CCN(C(=O)Cc2ccc(NC(=O)NCc3ccc(Cl)cc3)cc2)C(CO)C1] | {[(4- chlorophenyl)methyl]amino}- N-(4-{2-[2-(hydroxymethyl)-4- methylpiperazinyl]-2- oxoethyl}phenyl)carboxamide |
| 105 | 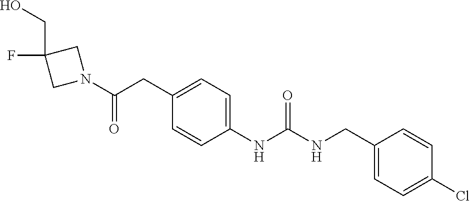 [SMILES: O=C(NCc1ccc(Cl)cc1)Nc1ccc(CC(=O)N2CC(F)(CO)C2)cc1] | N-[(4-chlorophenyl)methyl][(4- {2-[3-fluoro-3- (hydroxymethyl)azetidinyl]-2- oxoethyl}phenyl)amino] carboxamide |
| 106 | 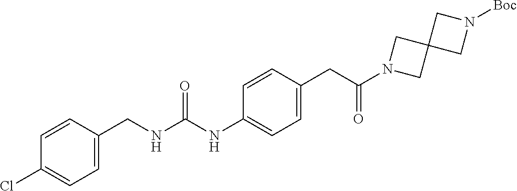 [SMILES: CC(C)(C)OC(=O)N1CC2(CN(C(=O)Cc3ccc(NC(=O)NCc4ccc(Cl)cc4)cc3)C2)C1] | tert-butyl 6-{2-[4-({[(4- chlorophenyl)methyl]amino} carbonylamino)phenyl]acetyl}-2,6- diazaspiro[3.3]heptane-2- carboxylate |
| 107 | 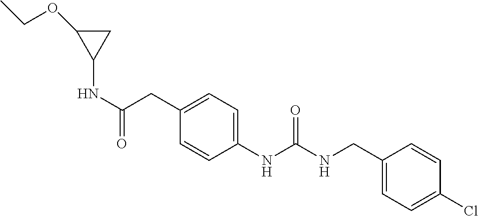 [SMILES: CCOC1CC1NC(=O)Cc1ccc(NC(=O)NCc2ccc(Cl)cc2)cc1] | 2-[4-({N-[(4- chlorophenyl)methyl]carbamoyl} amino)phenyl]-N-(2- ethoxycyclopropyl)acetamide |
| 108 | 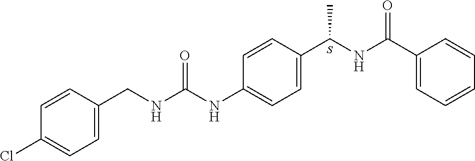 | ({4-[(1S)-1- (phenylcarbonylamino)ethyl] phenyl}amino)-N-[(4- chlorophenyl)methyl]carboxamide |
| 109 | 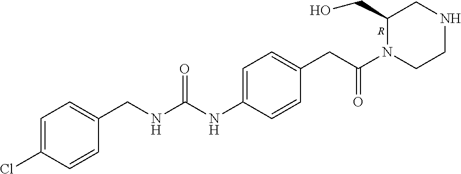 | N-(4-{2-[(2R)-2- (hydroxymethyl)piperazinyl]-2- oxoethyl}phenyl){[(4- chlorophenyl)methyl]amino} carboxamide |
| 110 | 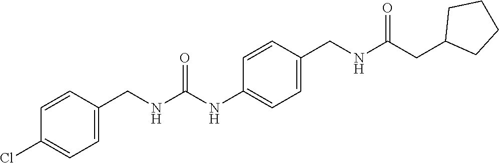 [SMILES: O=C(CC1CCCC1)NCc1ccc(NC(=O)NCc2ccc(Cl)cc2)cc1] | N-{[4-({[(4- chlorophenyl)methyl]amino}car bonylamino)phenyl]methyl}-2- cyclopentylacetamide |
| 111 | 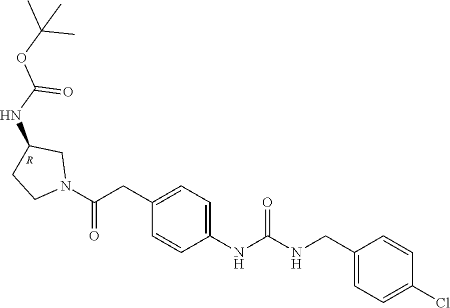 | {[4-(2-{(3R)-3-[(tert- butoxy)carbonylamino] pyrrolidinyl}-2- oxoethyl)phenyl]amino}-N-[(4- chlorophenyl)methyl]carboxamide |
| 112 | 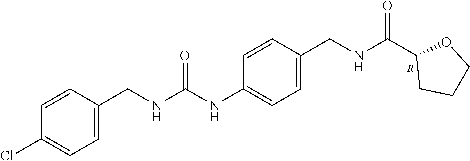 | N-(4-{[((2R)oxolan-2- yl)carbonylamino]methyl} phenyl){[(4- chlorophenyl)methyl]amino} carboxamide |
| 113 | 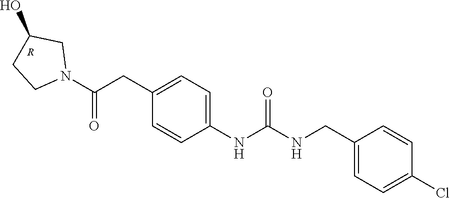 | ({4-[2-((3R)-3- hydroxypyrrolidinyl)-2- oxoethyl]phenyl}amino)-N-[(4- chlorophenyl)methyl]carboxamide |
| 114 | 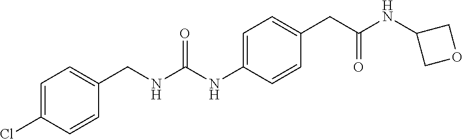 [SMILES: O=C(Cc1ccc(NC(=O)NCc2ccc(Cl)cc2)cc1)NC1COC1] | 2-[4-({[(4- chlorophenyl)methyl]amino} carbonylamino)phenyl]-N-oxetan- 3-ylacetamide |
| 115 | 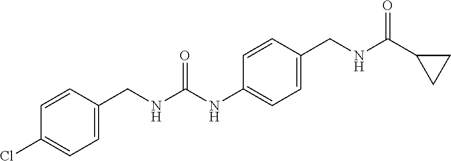 [SMILES: O=C(NCc1ccc(Cl)cc1)Nc1ccc(CNC(=O)C2CC2)cc1] | {[(4-chlorophenyl)methyl]amino}- N-{4-[(cyclopropylcarbonylamino) methyl]phenyl}carboxamide |
| 116 | 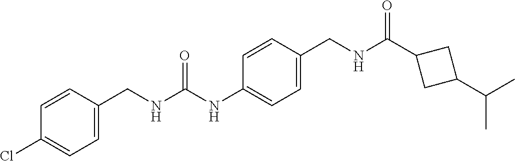 [SMILES: CC(C)C1CC(C(=O)NCc2ccc(NC(=O)NCc3ccc(Cl)cc3)cc2)C1] | {[(4- chlorophenyl)methyl]amino}- N-[4-({[3- (methylethyl)cyclobutyl] carbonylamino}methyl)phenyl] carboxamide |
| 117 | 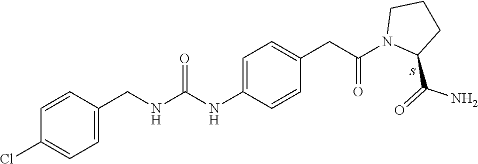 | (2S)-1-{2-[4-({[(4- olidine-2-carboxamide chlorophenyl)methyl]amino} carbonylamino)phenyl]acetyl} pyrrolidine-2-carboxamide |
| 118 | 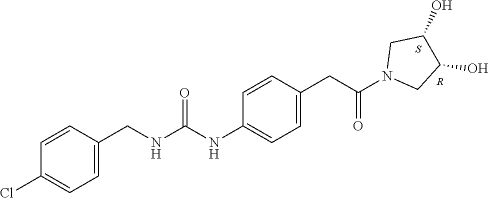 | N-{4-[2-((4S,3R)-3,4- dihydroxypyrrolidinyl)-2- oxoethyl]phenyl}{[(4- chlorophenyl)methyl]amino} carboxamide |
| 119 | 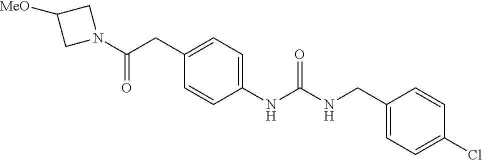 [SMILES: COC1CN(C(=O)Cc2ccc(NC(=O)NCc3ccc(Cl)cc3)cc2)C1] | N-[(4-chlorophenyl)methyl]({4- [2-(3-methoxyazetidinyl)-2- oxoethyl]phenyl}amino) carboxamide |
| 120 | 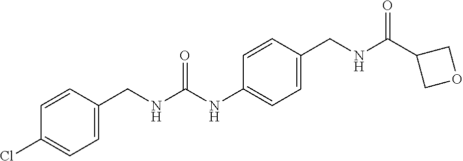 [SMILES: O=C(NCc1ccc(Cl)cc1)Nc1ccc(CNC(=O)C2COC2)cc1] | {[(4- chlorophenyl)methyl]amino}- N-{4-[(oxetan-3- ylcarbonylamino)methyl]phenyl} carboxamide |
| 121 | 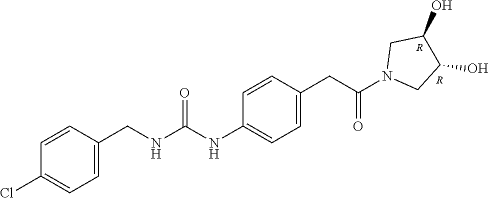 | N-{4-[2-((3R,4R)-3,4- dihydroxypyrrolidinyl)-2- oxoethyl]phenyl}{[(4- chlorophenyl)methyl]amino} carboxamide |
| 122 | 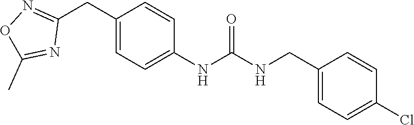 [SMILES: Cc1nc(Cc2ccc(NC(=O)NCc3ccc(Cl)cc3)cc2)no1] | {[(4- chlorophenyl)methyl]amino}- N-{4-[(5-methyl(1,2,4- oxadiazol-3- yl))methyl]phenyl}carboxamide |
| 123 | 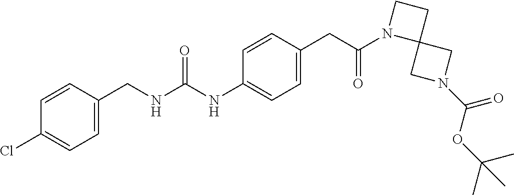 [SMILES: CC(C)(C)OC(=O)N1CC2(CCN2C(=O)Cc2ccc(NC(=O)NCc3ccc(Cl)cc3)cc2)C1] | tert-butyl 5-(2-[4-({[(4- chlorophenyl)methyl]amino} carbonylamino)phenyl]acetyl}-2,5- diazaspiro[3.3]heptane-2- carboxylate |
| 124 | 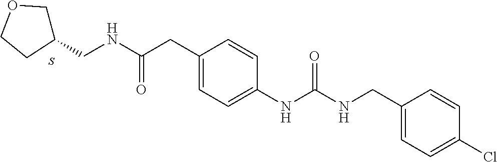 | N-[((3S)oxolan-3-yl)methyl]-2- [4-({[(4- chlorophenyl)methyl]amino} carbonylamino)phenyl]acetamide |
| 125 | 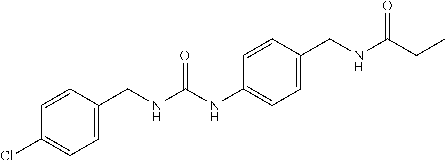 [SMILES: CCC(=O)NCc1ccc(NC(=O)NCc2ccc(Cl)cc2)cc1] | N-{[4-({[(4- chlorophenyl)methyl]amino} carbonylamino)phenyl]methyl} propanamide |
| 126 | 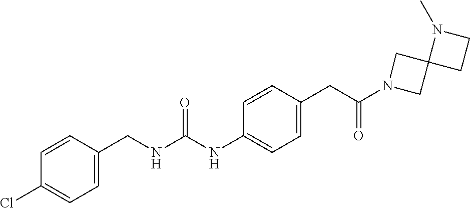 [SMILES: CN1CCC12CN(C(=O)Cc1ccc(NC(=O)NCc3ccc(Cl)cc3)cc1)C2] | N-[(4-chlorophenyl)methyl]({4- [2-(5-methyl-2,5- diazaspiro[3.3]hept-2-yl)-2- oxoethyl]phenyl}amino) carboxamide |
| 127 | 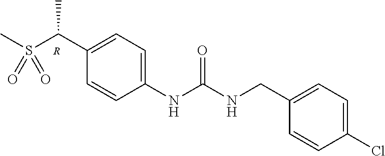 | N-(4-[(1R)-1- (methylsulfonyl)ethyl]phenyl} {[(4-chlorophenyl)methyl]amino} carboxamide |
| 128 | 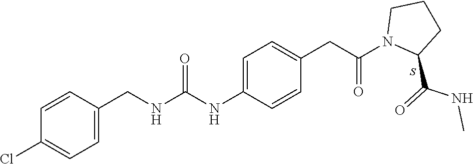 | N-(4-{2-[(2S)-2-(N- methylcarbamoyl)pyrrolidinyl]- 2-oxoethyl}phenyl){[(4- chlorophenyl)methyl]amino} carboxamide |
| 129 | 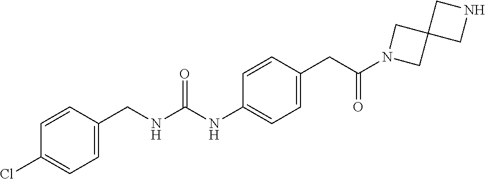 [SMILES: O=C(NCc1ccc(Cl)cc1)Nc1ccc(CC(=O)N2CC3(CNC3)C2)cc1] | N-{4-[2-(2,6- diazaspiro[3.3]hept-2-yl)-2- oxoethyl]phenyl}{[(4- chlorophenyl)methyl]amino} carboxamide |
| 130 | 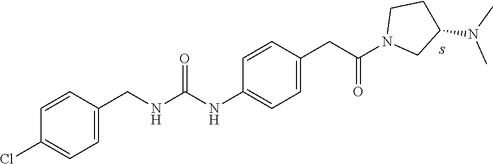 | N-(4-{2-[(3S)-3- (dimethylamino)pyrrolidinyl]- 2-oxoethyl}phenyl){[(4- chlorophenyl)methyl]amino} carboxamide |
| 131 | 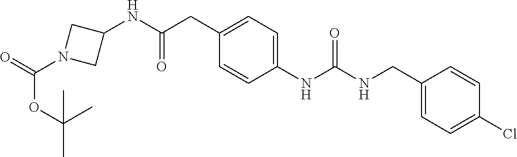 [SMILES: CC(C)(C)OC(=O)N1CC(NC(=O)Cc2ccc(NC(=O)NCc3ccc(Cl)cc3)cc2)C1] | tert-butyl 3-{2-[4-({N-[(4- chlorophenyl)methyl]carbamoyl} amino)phenyl]acetylamino} azetidinecarboxylate |
| 132 | 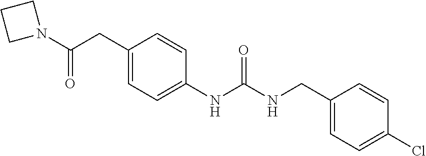 [SMILES: O=C(NCc1ccc(Cl)cc1)Nc1ccc(CC(=O)N2CCC2)cc1] | N-[4-(2-azetidinyl-2- oxoethyl)phenyl]{[(4- chlorophenyl)methyl]amino} carboxamide |
| 133 | 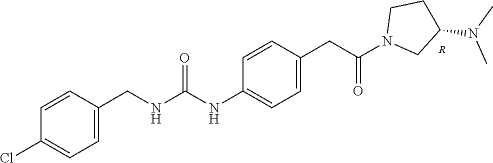 | N-(4-{2-[(3R)-3- (dimethylamino)pyrrolidinyl]- 2-oxoethyl}phenyl){[(4- chlorophenyl)methyl]amino} carboxamide |
| 134 | 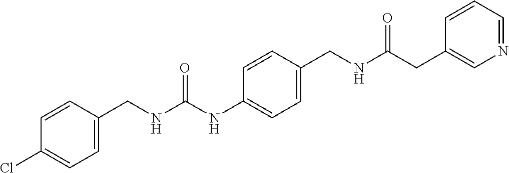 [SMILES: O=C(Cc1cccnc1)NCc1ccc(NC(=O)NCc2ccc(Cl)cc2)cc1] | N-{[4-({[(4- chlorophenyl)methyl]amino} carbonylamino)phenyl]methyl}-2- (3-pyridyl)acetamide |
| 135 | 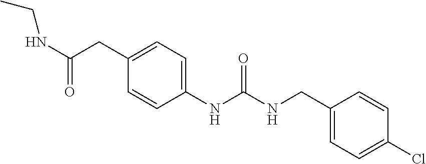 [SMILES: CCNC(=O)Cc1ccc(NC(=O)NCc2ccc(Cl)cc2)cc1] | 2-[4-({N-[(4- chlorophenyl)methyl]carbamoyl} amino)phenyl]-N- ethylacetamide |
| 136 | 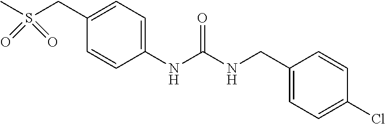 [SMILES: CS(=O)(=O)Cc1ccc(NC(=O)NCc2ccc(Cl)cc2)cc1] | N-[(4-chlorophenyl)methyl]({4- [(methylsulfonyl)methyl]phenyl} amino)carboxamide |
| 137 | 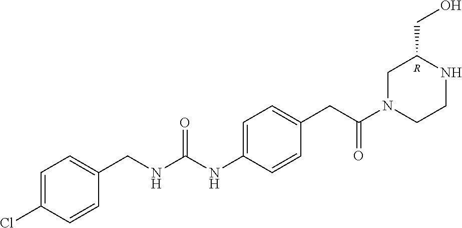 | N-(4-{2-[(3R)-3- (hydroxymethyl)piperazinyl]-2- oxoethyl}phenyl){[(4- chlorophenyl)methyl]amino} carboxamide |
| 138 | 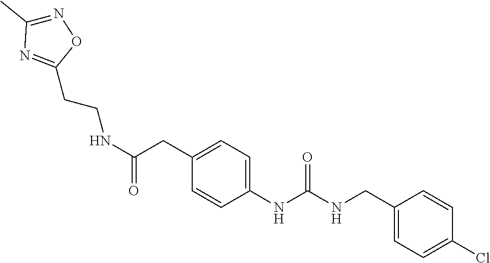 [SMILES: Cc1noc(CCNC(=O)Cc2ccc(NC(=O)NCc3ccc(Cl)cc3)cc2)n1] | 2-[4-({N-[(4- chlorophenyl)methyl]carbamoyl} amino)phenyl]-N-[2-(3- methyl(1,2,4-oxadiazol-5- yl))ethyl]acetamide |
| 139 | 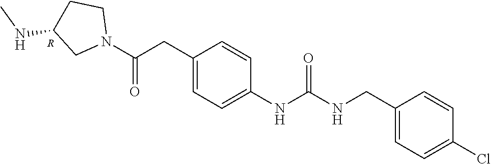 | [(4-{2-[(3R)-3- (methylamino)pyrrolidinyl]-2- oxoethyl}phenyl)amino]-N-[(4- chlorophenyl)methyl]carboxamide |
| 140 | 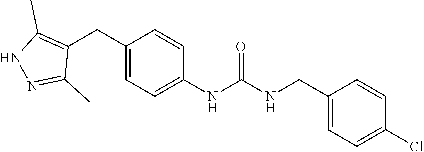 [SMILES: Cc1n[nH]c(C)c1Cc1ccc(NC(=O)NCc2ccc(Cl)cc2)cc1] | N-{4-[(3,5-dimethylpyrazol-4- yl)methyl]phenyl}{[(4- chlorophenyl)methyl]amino} carboxamide |
| 141 | 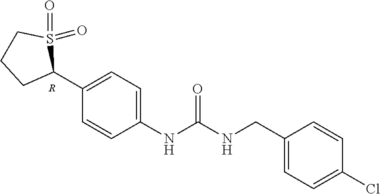 | {[4-((2R)-1,1-dioxothiolan-2- yl)phenyl]amino}-N-[(4- chlorophenyl)methyl]carboxamide |
| 142 | 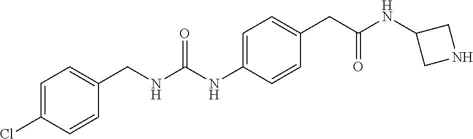 [SMILES: O=C(Cc1ccc(NC(=O)NCc2ccc(Cl)cc2)cc1)NC1CNC1] | N-azetidin-3-yl-2-[4-({[(4- chlorophenyl)methyl]amino}car bonylamino)phenyl]acetamide |
| 143 | 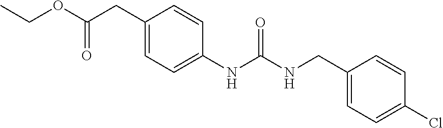 [SMILES: CCOC(=O)Cc1ccc(NC(=O)NCc2ccc(Cl)cc2)cc1] | ethyl 2-[4-({[(4- chlorophenyl)methyl]amino} carbonylamino)phenyl]acetate |
| 144 | 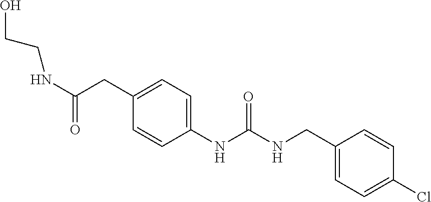 [SMILES: O=C(Cc1ccc(NC(=O)NCc2ccc(Cl)cc2)cc1)NCCO] | 2-[4-({N-[(4- chlorophenyl)methyl]carbamoyl} amino)phenyl]-N-(2- hydroxyethyl)acetamide |
| 145 | 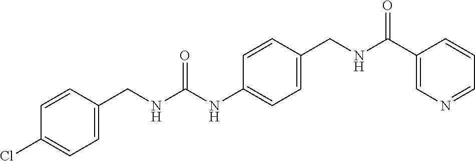 [SMILES: O=C(NCc1ccc(Cl)cc1)Nc1ccc(CNC(=O)c2cccnc2)cc1] | {[(4-chlorophenyl)methyl]amino}- N-{4-[(3- pyridylcarbonylamino)methyl] phenyl}carboxamide |
| 146 | 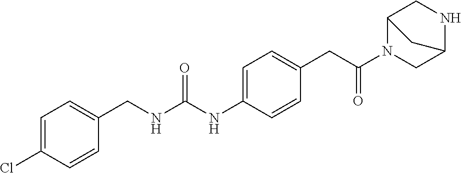 [SMILES: O=C(NCc1ccc(Cl)cc1)Nc1ccc(CC(=O)N2CC3CC2CN3)cc1] | N-(4-[2-(2,5- diazabicyclo[2.2.1]hept-2-yl)-2- oxoethyl]phenyl}{[(4- chlorophenyl)methyl]amino} carboxamide |
| 147 | 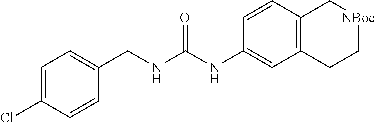 [SMILES: CC(C)(C)OC(=O)N1CCc2cc(NC(=O)NCc3ccc(Cl)cc3)ccc2C1] | tert-butyl 6-({N-[(4- chlorophenyl)methyl]carbamoyl} amino)-1,2,3,4- tetrahydroisoquinoline-2- carboxylate |
| 148 | 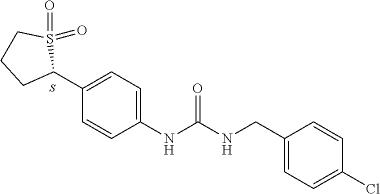 | {[4-((2S)-1,1-dioxothiolan-2- yl)phenyl]amino}-N-[(4- chlorophenyl)methyl]carboxamide |
| 149 | 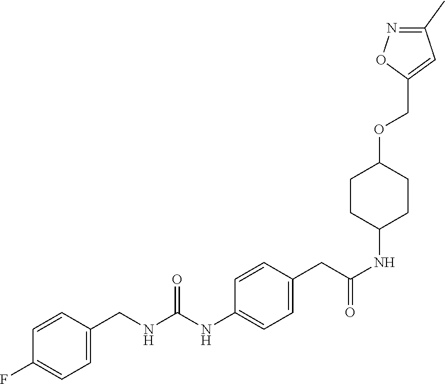 [SMILES: Cc1cc(COC2CCC(NC(=O)Cc3ccc(NC(=O)NCc4ccc(F)cc4)cc3)CC2)on1] | 2-[4-({[(4- fluorophenyl)methyl]amino} carbonylamino)phenyl]-N-{4-[(3- methylisoxazol-5- yl)methoxy]cyclohexyl}acetamide |
| 150 | 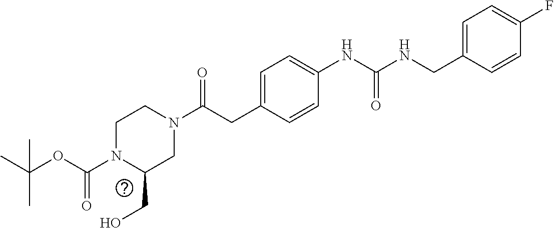 [SMILES: CC(C)(C)OC(=O)N1CCN(C(=O)Cc2ccc(NC(=O)NCc3ccc(F)cc3)cc2)C[C@H]1CO] | tert-butyl (2S)-4-{2-[4-({[(4- fluorophenyl)methyl]amino} carbonylamino)phenyl]acetyl}-2- (hydroxymethyl) piperazinecarboxylate |
| 151 | 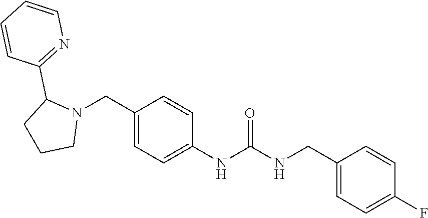 [SMILES: O=C(NCc1ccc(F)cc1)Nc1ccc(CN2CCCC2c2ccccn2)cc1] | N-[(4-fluorophenyl)methyl]({4- [(2-(2- pyridyl)pyrrolidinyl)methyl] phenyl}amino)carboxamide |
| 152 | 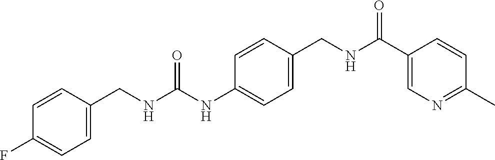 [SMILES: Cc1ccc(C(=O)NCc2ccc(NC(=O)NCc3ccc(F)cc3)cc2)cn1] | {[(4-fluorophenyl)methyl]amino}- N-(4-{[(6-methyl(3- pyridyl))carbonylamino]methyl} phenyl)carboxamide |
| 153 | 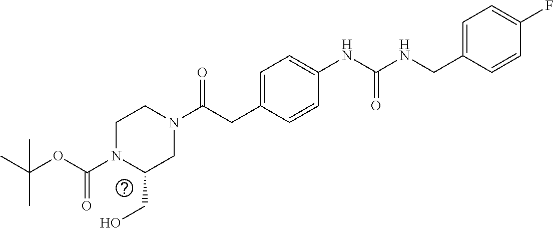 [SMILES: CC(C)(C)OC(=O)N1CCN(C(=O)Cc2ccc(NC(=O)NCc3ccc(F)cc3)cc2)C[C@@H]1CO] | tert-butyl (2R)-4-{2-[4-({[(4- fluorophenyl)methyl]amino} carbonylamino)phenyl]acetyl}-2- (hydroxymethyl) piperazinecarboxylate |
| 154 | 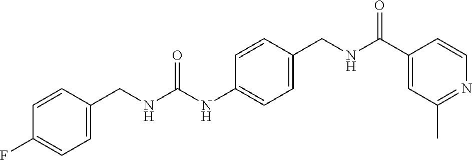 [SMILES: Cc1cc(C(=O)NCc2ccc(NC(=O)NCc3ccc(F)cc3)cc2)ccn1] | {[(4-fluorophenyl)methyl]amino}- N-(4-{[(2-methyl(4- pyridyl))carbonylamino]methyl} phenyl)carboxamide |
| 155 | 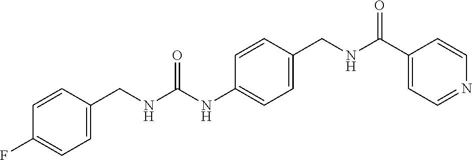 [SMILES: O=C(NCc1ccc(F)cc1)Nc1ccc(CNC(=O)c2ccncc2)cc1] | {[(4-fluorophenyl)methyl]amino}- N-{4-[(4- pyridylcarbonylamino)methyl] phenyl}carboxamide |
| 156 | 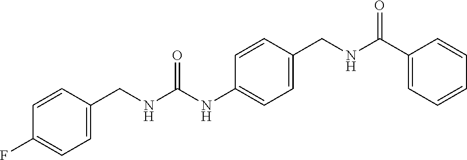 [SMILES: O=C(NCc1ccc(F)cc1)Nc1ccc(CNC(=O)c2ccccc2)cc1] | {[(4-fluorophenyl)methyl]amino}- N-{4- [(phenylcarbonylamino)methyl] phenyl}carboxamide |
| 157 | 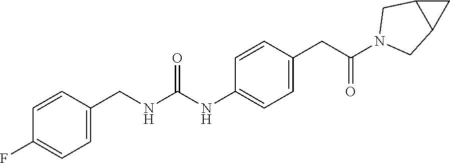 [SMILES: O=C(NCc1ccc(F)cc1)Nc1ccc(CC(=O)N2CC3CC3C2)cc1] | N-{4-[2-(3- azabicyclo[3.1.0]hex-3-yl)-2- oxoethyl]phenyl}{[(4- fluorophenyl)methyl]amino} carboxamide |
| 158 | 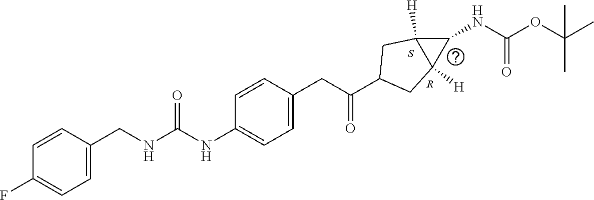 | N-[4-(2-{(5S,1R)-6-[(tert- butoxy)carbonylamino]-3- azabicyclo[3.1.0]hex-3-yl}-2- oxoethyl)phenyl]{[(4- fluorophenyl)methyl]amino} carboxamide |
| 159 | 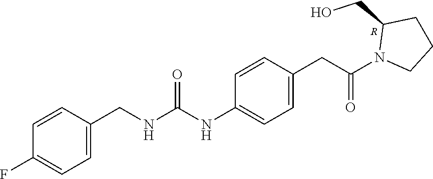 | N-(4-(2-[(2R)-2- (hydroxymethyl)pyrrolidinyl]- 2-oxoethyl}phenyl){[(4- fluorophenyl)methyl]amino} carboxamide |
| 160 | 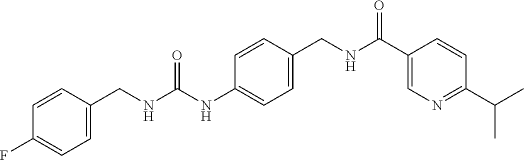 [SMILES: CC(C)c1ccc(C(=O)NCc2ccc(NC(=O)NCc3ccc(F)cc3)cc2)cn1] | {[(4-fluorophenyl)methyl]amino}- N-[4-({[6-(methylethyl)(3- pyridyl)]carbonylamino}methyl) phenyl]carboxamide |
| 161 | 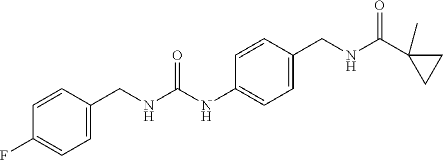 [SMILES: CC1(C(=O)NCc2ccc(NC(=O)NCc3ccc(F)cc3)cc2)CC1] | {[(4-fluorophenyl)methyl]amino}- N-(4- {[(methylcyclopropyl)carbonyl- amino]methyl}phenyl) carboxamide |
| 162 | 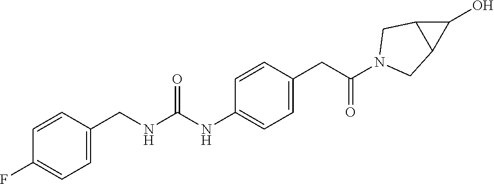 [SMILES: O=C(NCc1ccc(F)cc1)Nc1ccc(CC(=O)N2CC3C(O)C3C2)cc1] | {[(4-fluorophenyl)methyl]amino}- N-{4-[2-(6-hydroxy-3- azabicyclo[3.1.0]hex-3-yl)-2- oxoethyl]phenyl}carboxamide |
| 163 | 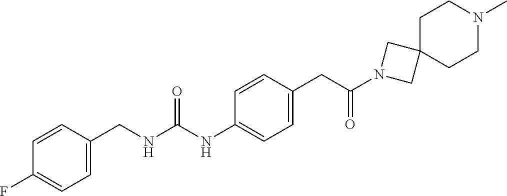 [SMILES: CN1CCC2(CC1)CN(C(=O)Cc1ccc(NC(=O)NCc3ccc(F)cc3)cc1)C2] | {[(4-fluorophenyl)methyl]amino}- N-{4-[2-(7-methyl-2,7- diazaspiro[3.5]non-2-yl)-2- oxoethyl]phenyl}carboxamide |
| 164 | 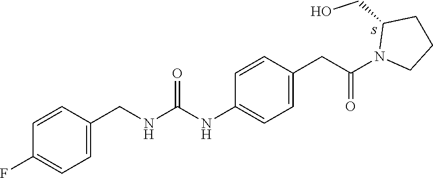 | N-(4-(2-[(2S)-2- (hydroxymethyl)pyrrolidinyl]- 2-oxoethyl}phenyl){[(4- fluorophenyl)methyl]amino} carboxamide |
| 165 | 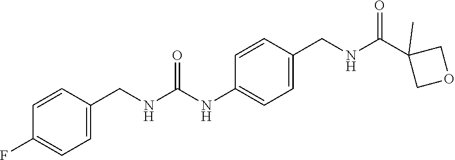 [SMILES: CC1(C(=O)NCc2ccc(NC(=O)NCc3ccc(F)cc3)cc2)COC1] | {[(4- fluorophenyl)methyl]amino}- N-(4-{[(3-methyloxetan-3- yl)carbonylamino]methyl} phenyl)carboxamide |
| 166 | 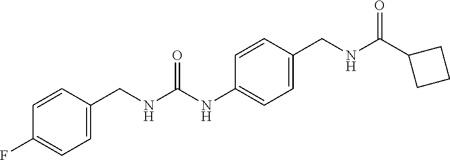 [SMILES: O=C(NCc1ccc(F)cc1)Nc1ccc(CNC(=O)C2CCC2)cc1] | N-{4- [(cyclobutylcarbonylamino) methyl]phenyl}{[(4- fluorophenyl)methyl]amino} carboxamide |
| 167 | 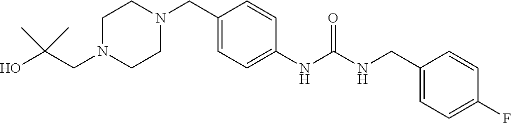 [SMILES: CC(C)(O)CN1CCN(Cc2ccc(NC(=O)NCc3ccc(F)cc3)cc2)CC1] | {[(4-fluorophenyl)methyl]amino}- N-(4-{[4-(2-hydroxy-2- methylpropyl)piperazinyl] methyl}phenyl)carboxamide |
| 168 | 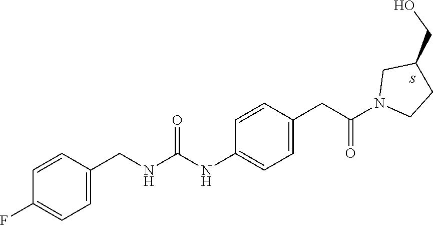 | N-(4-{2-[(3S)-3- (hydroxymethyl)pyrrolidinyl]- 2-oxoethyl}phenyl){[(4- fluorophenyl)methyl]amino} carboxamide |
| 169 | 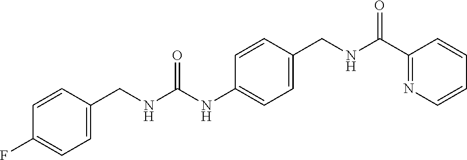 [SMILES: O=C(NCc1ccc(F)cc1)Nc1ccc(CNC(=O)c2ccccn2)cc1] | {[(4- fluorophenyl)methyl]amino}- N-{4-[(2- pyridylcarbonylamino)methyl] phenyl}carboxamide |
| 170 | 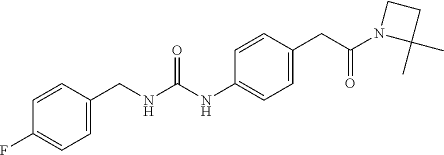 [SMILES: CC1(C)CCN1C(=O)Cc1ccc(NC(=O)NCc2ccc(F)cc2)cc1] | ({4-[2-(2,2-dimethylazetidinyl)- 2-oxoethyl]phenyl}amino)-N- [(4-fluorophenyl)methyl] carboxamide |
| 171 | 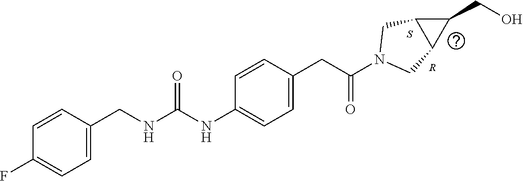 | [(4-{2-[(5S,1R)-6- (hydroxymethyl)-3- azabicyclo[3.1.0]hex-3-yl]-2- oxoethyl}phenyl)amino]-N-[(4- fluorophenyl)methyl]carboxamide |
| 172 | 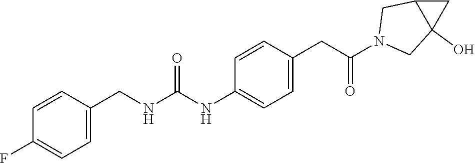 [SMILES: O=C(NCc1ccc(F)cc1)Nc1ccc(CC(=O)N2CC3CC3(O)C2)cc1] | N-[(4-fluorophenyl)methyl]({4- [2-(1-hydroxy-3- azabicyclo[3.1.0]hex-3-yl)-2- oxoethyl]phenyl}amino) carboxamide |
| 173 | 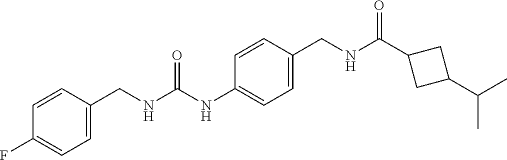 [SMILES: CC(C)C1CC(C(=O)NCc2ccc(NC(=O)NCc3ccc(F)cc3)cc2)C1] | {[(4-fluorophenyl)methyl]amino}- N-[4-({[3- (methylethyl)cyclobutyl] carbonylamino}methyl)phenyl] carboxamide |
| 174 | 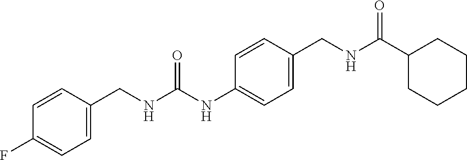 [SMILES: O=C(NCc1ccc(F)cc1)Nc1ccc(CNC(=O)C2CCCCC2)cc1] | N-{4- [(cyclohexylcarbonylamino) methyl]phenyl}{[(4- fluorophenyl)methyl]amino} carboxamide |
| 175 | 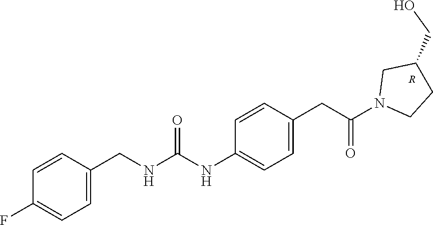 | N-(4-{2-[(3R)-3- (hydroxymethyl)pyrrolidinyl]- 2-oxoethyl}phenyl){[(4- fluorophenyl)methyl]amino} carboxamide |
| 176 | 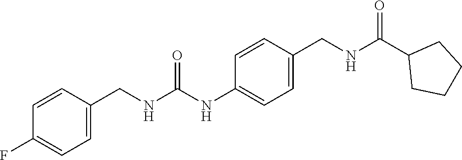 [SMILES: O=C(NCc1ccc(F)cc1)Nc1ccc(CNC(=O)C2CCCC2)cc1] | N-{4- [(cyclopentylcarbonylamino) methyl]phenyl}{[(4- fluorophenyl)methyl]amino} carboxamide |
| 177 | 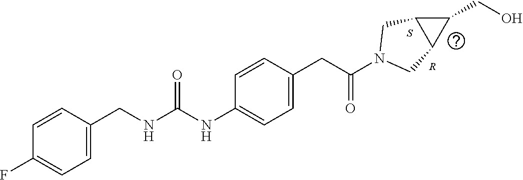 | [(4-{2-[(5S,1R)-6- (hydroxymethyl)-3- azabicyclo[3.1.0]hex-3-yl]-2- oxoethyl}phenyl)amino]-N-[(4- fluorophenyl)methyl]carboxamide |
| 178 | 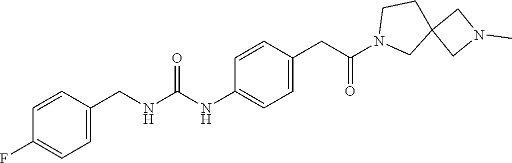 [SMILES: CN1CC2(CCN(C(=O)Cc3ccc(NC(=O)NCc4ccc(F)cc4)cc3)C2)C1] | {[(4- fluorophenyl)methyl]amino}- N-{4-[2-(2-methyl-2,6- diazaspiro[3.4]oct-6-yl)-2- oxoethyl]phenyl}carboxamide |
| 179 | 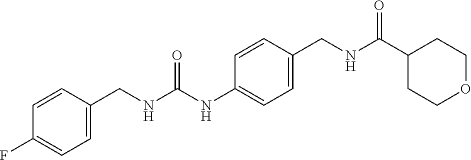 [SMILES: O=C(NCc1ccc(F)cc1)Nc1ccc(CNC(=O)C2CCOCC2)cc1] | {[(4- fluorophenyl)methyl]amino}- N-{4-[(2H-3,4,5,6- tetrahydropyran-4- ylcarbonylamino)methyl]phenyl} carboxamide |
| 180 | 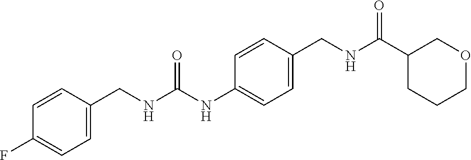 [SMILES: O=C(NCc1ccc(F)cc1)Nc1ccc(CNC(=O)C2CCCOC2)cc1] | {[(4- fluorophenyl)methyl]amino}- N-{4-[(2H-3,4,5,6- tetrahydropyran-3- ylcarbonylamino)methyl]phenyl} carboxamide |
| 181 | 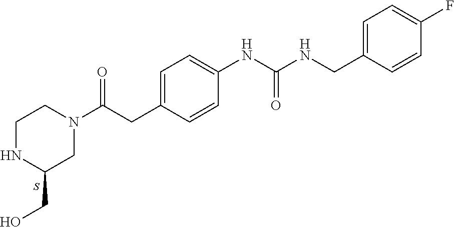 | N-(4-{2-[(3S)-3- (hydroxymethyl)piperazinyl]-2- oxoethyl}phenyl){[(4- fluorophenyl)methyl]amino} carboxamide |
| 182 | 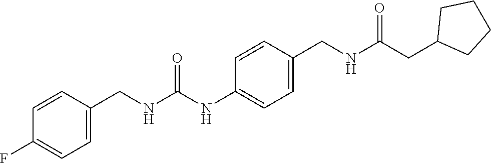 [SMILES: O=C(CC1CCCC1)NCc1ccc(NC(=O)NCc2ccc(F)cc2)cc1] | 2-cyclopentyl-N-{[4-({[(4- fluorophenyl)methyl]amino} carbonylamino)phenyl]methyl} acetamide |
| 183 | 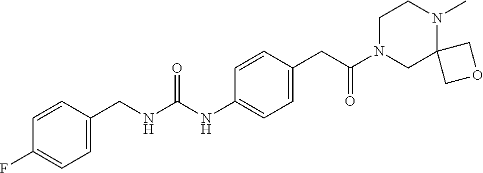 [SMILES: CN1CCN(C(=O)Cc2ccc(NC(=O)NCc3ccc(F)cc3)cc2)CC12COC2] | {[(4- fluorophenyl)methyl]amino}- N-{4-[2-(9-methyl-2-oxa-6,9- diazaspiro[3.5]non-6-yl)-2- oxoethyl]phenyl}carboxamide |
| 184 | 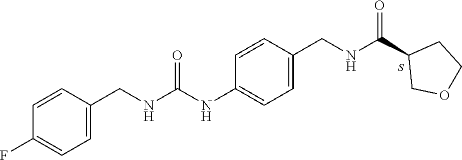 | N-(4-{[((3S)oxolan-3- yl)carbonylamino]methyl} phenyl){[(4- fluorophenyl)methyl]amino} carboxamide |
| 185 | 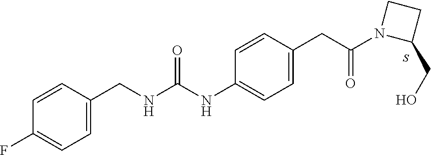 | N-(4-{2-[(2S)-2- (hydroxymethyl)azetidinyl]-2- oxoethyl}phenyl){[(4- fluorophenyl)methyl]amino} carboxamide |
| 186 | 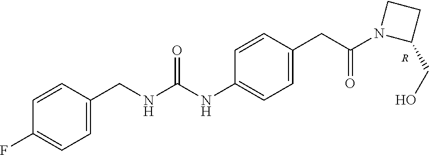 | N-(4-{2-[(2R)-2- (hydroxymethyl)azetidinyl]-2- oxoethyl}phenyl){[(4- fluorophenyl)methyl]amino} carboxamide |
| 187 | 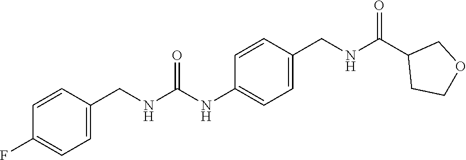 [SMILES: O=C(NCc1ccc(F)cc1)Nc1ccc(CNC(=O)C2CCOC2)cc1] | {[(4- fluorophenyl)methyl]amino}- N-{4-[(oxolan-3- ylcarbonylamino)methyl]phenyl} carboxamide |
| 188 | 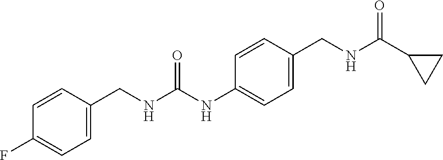 [SMILES: O=C(NCc1ccc(F)cc1)Nc1ccc(CNC(=O)C2CC2)cc1] | N-{4- [(cyclopropylcarbonylamino) methyl]phenyl}{[(4- fluorophenyl)methyl]amino} carboxamide |
| 189 | 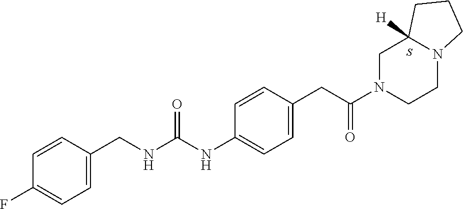 | N-(4-[2-((1S)-3,6- diazabicyclo[4.3.0]non-3-yl)-2- oxoethyl]phenyl]{[(4- fluorophenyl)methyl]amino} carboxamide |
| 190 | 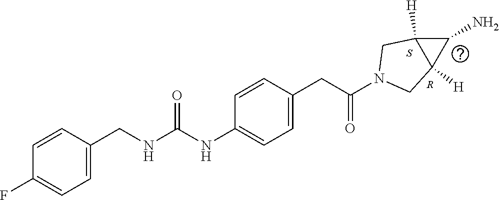 | N-{4-[2-((5S,1R)-6-amino-3- azabicyclo[3.1.0]hex-3-yl)-2- oxoethyl]phenyl]{[(4- fluorophenyl)methyl]amino} carboxamide |
| 191 | 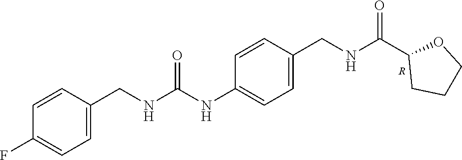 | N-(4-{[((2R)oxolan-2- yl)carbonylamino]methyl} phenyl){[(4- fluorophenyl)methyl]amino} carboxamide |
| 192 | 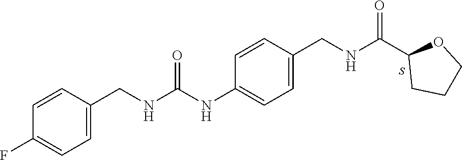 | N-(4-{[((2S)oxolan-2- yl)carbonylamino]methyl} phenyl){[(4- fluorophenyl)methyl]amino} carboxamide |
| 193 | 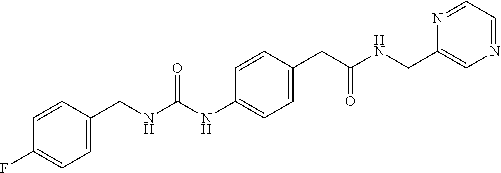 [SMILES: O=C(Cc1ccc(NC(=O)NCc2ccc(F)cc2)cc1)NCc1cnccn1] | 2-[4-({N-[(4- fluorophenyl)methyl]carbamoyl} amino)phenyl]-N-(pyrazin-2- ylmethyl)acetamide |
| 194 | 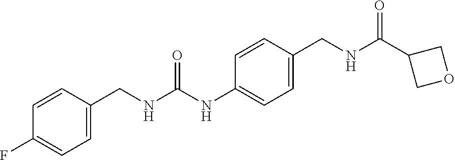 [SMILES: O=C(NCc1ccc(F)cc1)Nc1ccc(CNC(=O)C2COC2)cc1] | {[(4- fluorophenyl)methyl]amino}- N-{4-[(oxetan-3- ylcarbonylamino)methyl]phenyl} carboxamide |
| 195 | 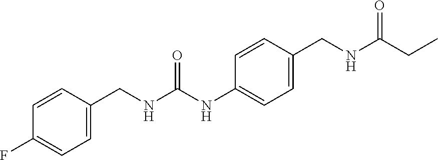 [SMILES: CCC(=O)NCc1ccc(NC(=O)NCc2ccc(F)cc2)cc1] | N-{[4-({[(4- fluorophenyl)methyl]amino} carbonylamino)phenyl]methyl} propanamide |
| 196 | 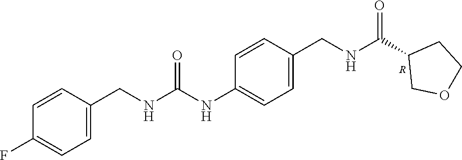 | N-(4-{[((3R)oxolan-3- yl)carbonylamino]methyl} phenyl){[(4- fluorophenyl)methyl]amino} carboxamide |
| 197 | 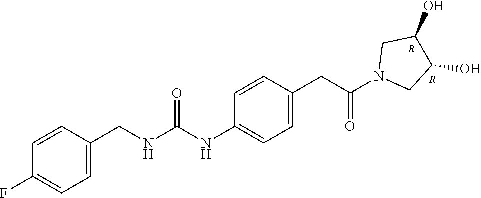 | N-{4-[2-((3R,4R)-3,4- dihydroxypyrrolidinyl)-2- oxoethyl]phenyl}{[(4- fluorophenyl)methyl]amino} carboxamide |
| 198 | 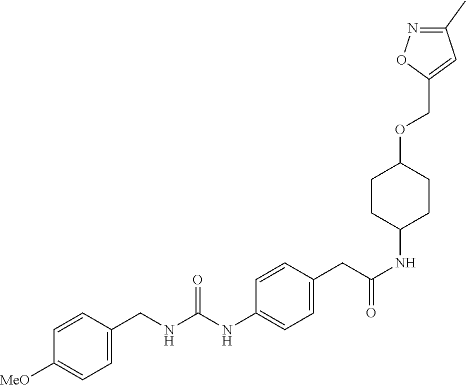 [SMILES: COc1ccc(CNC(=O)Nc2ccc(CC(=O)NC3CCC(OCc4cc(C)no4)CC3)cc2)cc1] | 2-[4-({N-[(4- methoxyphenyl)methyl]carbamoyl} amino)phenyl]-N-{4-[(3- methylisoxazol-5- yl)methoxy]cyclohexyl}acetamide |
| 199 | 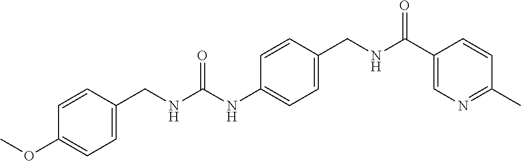 | {[(4- methoxyphenyl)methyl]amino}- N-(4-{[(6-methyl(3- pyridyl))carbonylamino]methyl} phenyl)carboxamide |
| 200 | 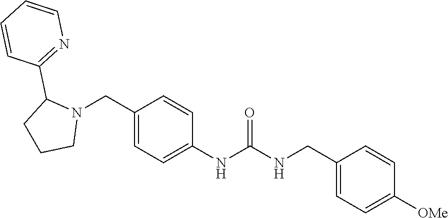 [SMILES: COc1ccc(CNC(=O)Nc2ccc(CN3CCCC3c3ccccn3)cc2)cc1] | {[(4- methoxyphenyl)methyl]amino}- N-{4-[(2-(2- pyridyl)pyrrolidinyl)methyl] phenyl}carboxamide |
| 201 | 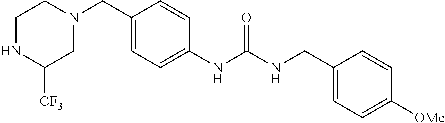 [SMILES: COc1ccc(CNC(=O)Nc2ccc(CN3CCNC(C(F)(F)F)C3)cc2)cc1] | {[(4- methoxyphenyl)methyl]amino}- N-(4-{[3- (trifluoromethyl)piperazinyl] methyl}phenyl)carboxamide |
| 202 | 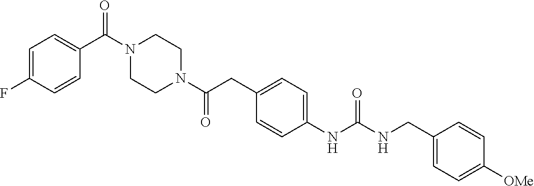 [SMILES: COc1ccc(CNC(=O)Nc2ccc(CC(=O)N3CCN(C(=O)c4ccc(F)cc4)CC3)cc2)cc1] | {[4-(2-{4-[(4- fluorophenyl)carbonyl] piperazinyl}-2- oxoethyl)phenyl]amino}-N-[(4- methoxyphenyl)methyl] carboxamide |
| 203 | 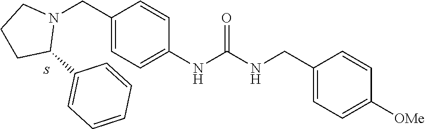 | N-{4-[((2S)-2- phenylpyrrolidinyl)methyl] phenyl}{[(4- methoxyphenyl)methyl]amino} carboxamide |
| 204 | 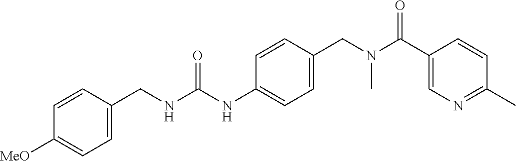 [SMILES: COc1ccc(CNC(=O)Nc2ccc(CN(C)C(=O)c3ccc(C)nc3)cc2)cc1] | {[(4- methoxyphenyl)methyl]amino}- N-(4-{[N-methyl(6-methyl(3- pyridyl))carbonylamino]methyl} phenyl)carboxamide |
| 205 | 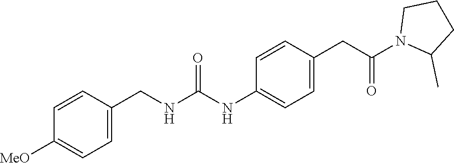 [SMILES: COc1ccc(CNC(=O)Nc2ccc(CC(=O)N3CCCC3C)cc2)cc1] | {[(4- methoxyphenyl)methyl]amino}- N-{4-[2-(2- methylpyrrolidinyl)-2- oxoethyl]phenyl}carboxamide |
| 206 | 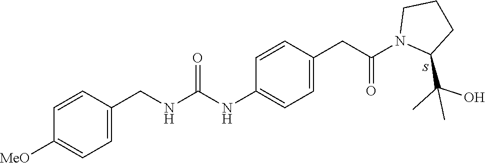 | N-(4-(2-[(2S)-2-(1-hydroxy- isopropyl)pyrrolidinyl]-2- oxoethyl}phenyl){[(4- methoxyphenyl)methyl]amino} carboxamide |
| 207 | 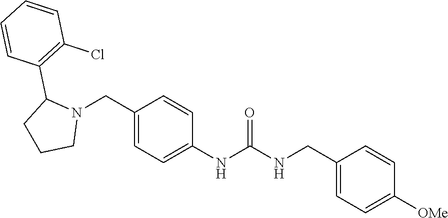 [SMILES: COc1ccc(CNC(=O)Nc2ccc(CN3CCCC3c3ccccc3Cl)cc2)cc1] | N-(4-{[2-(2- chlorophenyl)pyrrolidinyl] methyl}phenyl){[(4- methoxyphenyl)methyl]amino} carboxamide |
| 208 | 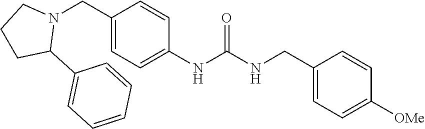 [SMILES: COc1ccc(CNC(=O)Nc2ccc(CN3CCCC3c3ccccc3)cc2)cc1] | {[(4- methoxyphenyl)methyl]amino}- N-{4-[(2- phenylpyrrolidinyl)methyl] phenyl}carboxamide |
| 209 | 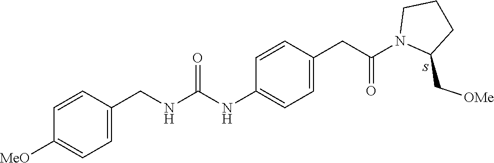 | N-(4-{2-[(2S)-2- (methoxymethyl)pyrrolidinyl]- 2-oxoethyl}phenyl){[(4- methoxyphenyl)methyl]amino} carboxamide |
| 210 | 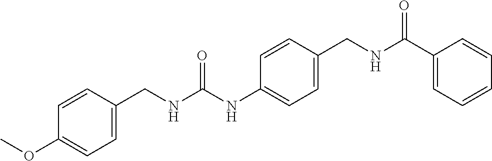 [SMILES: COc1ccc(CNC(=O)Nc2ccc(CNC(=O)c3ccccc3)cc2)cc1] | {[(4- methoxyphenyl)methyl]amino}- N-{4- [(phenylcarbonylamino)methyl] phenyl}carboxamide |
| 211 | 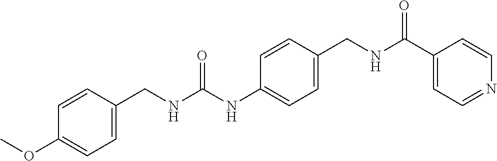 [SMILES: COc1ccc(CNC(=O)Nc2ccc(CNC(=O)c3ccncc3)cc2)cc1] | {[(4- methoxyphenyl)methyl]amino}- N-{4-[(4- pyridylcarbonylamino)methyl] phenyl}carboxamide |
| 212 | 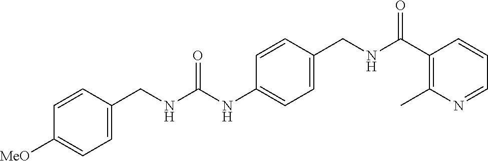 [SMILES: COc1ccc(CNC(=O)Nc2ccc(CNC(=O)c3cccnc3C)cc2)cc1] | {[(4- methoxyphenyl)methyl]amino}- N-(4-{[(2-methyl(3- pyridyl))carbonylamino]methyl} phenyl)carboxamide |
| 213 | 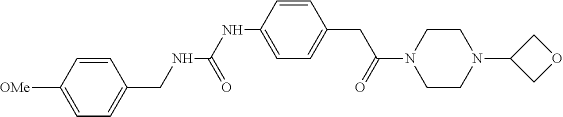 [SMILES: COc1ccc(CNC(=O)Nc2ccc(CC(=O)N3CCN(C4COC4)CC3)cc2)cc1] | N-[(4- methoxyphenyl)methyl]({4-[2- (4-oxetan-3-ylpiperazinyl)-2- oxoethyl]phenyl}amino) carboxamide |
| 214 | 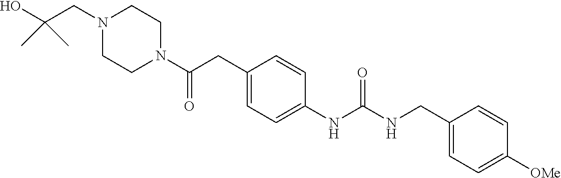 [SMILES: COc1ccc(CNC(=O)Nc2ccc(CC(=O)N3CCN(CC(C)(C)O)CC3)cc2)cc1] | N-(4-{2-[4-(2-hydroxy-2- methylpropyl)piperazinyl]-2- oxoethyl}phenyl){[(4- methoxyphenyl)methyl]amino} carboxamide |
| 215 | 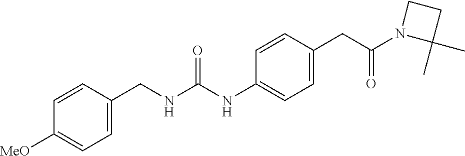 [SMILES: COc1ccc(CNC(=O)Nc2ccc(CC(=O)N3CCC3(C)C)cc2)cc1] | ({4-[2-(2,2-dimethylazetidinyl)- 2-oxoethyl]phenyl}amino)-N- [(4-methoxyphenyl)methyl] carboxamide |
| 216 | 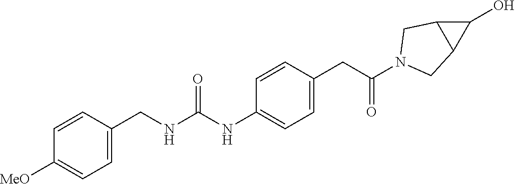 [SMILES: COc1ccc(CNC(=O)Nc2ccc(CC(=O)N3CC4C(O)C4C3)cc2)cc1] | ({4-[2-(6-hydroxy-3- azabicyclo[3.1.0]hex-3-yl)-2- oxoethyl]phenyl}amino)-N-[(4- methoxyphenyl)methyl] carboxamide |
| 217 | 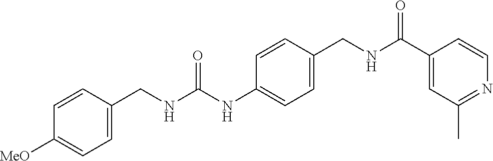 [SMILES: COc1ccc(CNC(=O)Nc2ccc(CNC(=O)c3ccnc(C)c3)cc2)cc1] | {[(4- methoxyphenyl)methyl]amino}- N-(4-{[(2-methyl(4- pyridyl))carbonylamino]methyl} phenyl)carboxamide |
| 218 | 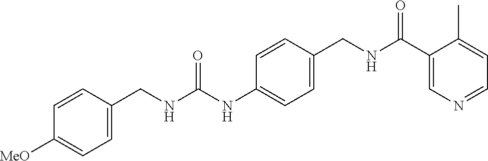 [SMILES: COc1ccc(CNC(=O)Nc2ccc(CNC(=O)c3cnccc3C)cc2)cc1] | {[(4- methoxyphenyl)methyl]amino}- N-(4-{[(4-methyl(3- pyridyl))carbonylamino]methyl} phenyl)carboxamide |
| 219 | 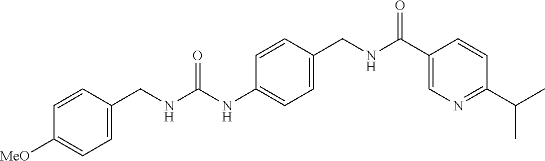 [SMILES: COc1ccc(CNC(=O)Nc2ccc(CNC(=O)c3ccc(C(C)C)nc3)cc2)cc1] | {[(4- methoxyphenyl)methyl]amino}- N-[4-({[6-(methylethyl)(3- pyridyl)]carbonylamino}methyl) phenyl]carboxamide |
| 220 | 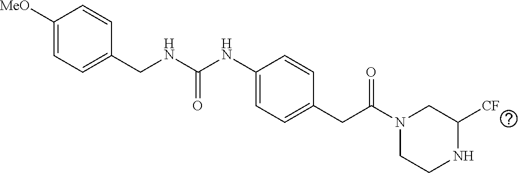 | N-[(4- methoxyphenyl)methyl][(4-{2- oxo-2-[3- (trifluoromethyl)piperazinyl] ethyl}phenyl)amino]carboxamide |
| 221 | 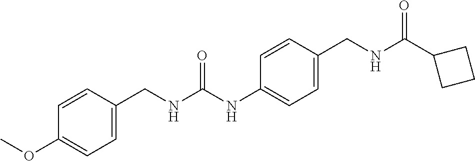 [SMILES: COc1ccc(CNC(=O)Nc2ccc(CNC(=O)C3CCC3)cc2)cc1] | N-{4- [(cyclobutylcarbonylamino) methyl]phenyl}{[(4- methoxyphenyl)methyl]amino} carboxamide |
| 222 | 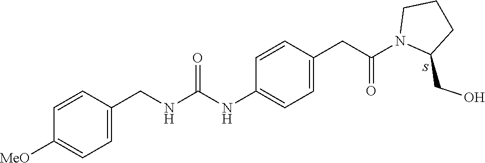 | N-(4-{2-[(2S)-2- (hydroxymethyl)pyrrolidinyl]- 2-oxoethyl}phenyl){[(4- methoxyphenyl)methyl]amino} carboxamide |
| 223 | 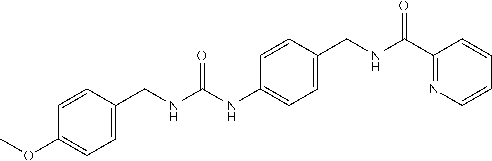 [SMILES: COc1ccc(CNC(=O)Nc2ccc(CNC(=O)c3ccccn3)cc2)cc1] | {[(4- methoxyphenyl)methyl]amino}- N-{4-[(2- pyridylcarbonylamino)methyl] phenyl}carboxamide |
| 224 | 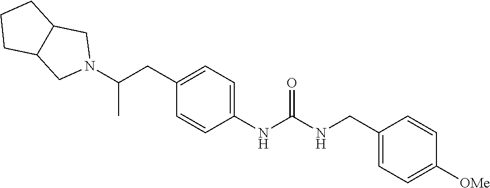 [SMILES: COc1ccc(CNC(=O)Nc2ccc(CC(C)N3CC4CCCC4C3)cc2)cc1] | ({4-[2-(3-azabicyclo[3.3.0]oct- 3-yl)-2- oxoethyl]phenyl}amino)-N-[(4- methoxyphenyl)methyl] carboxamide |
| 225 | 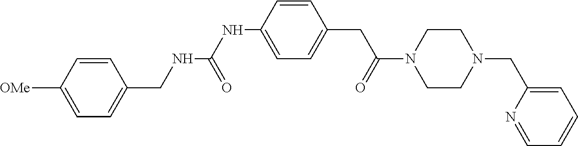 [SMILES: COc1ccc(CNC(=O)Nc2ccc(CC(=O)N3CCN(Cc4ccccn4)CC3)cc2)cc1] | N-[(4- methoxyphenyl)methyl][(4-{2- oxo-2-[4-(2- pyridylmethyl)piperazinyl]ethyl }phenyl)amino]carboxamide |
| 226 | 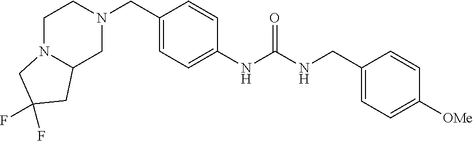 [SMILES: COc1ccc(CNC(=O)Nc2ccc(CN3CCN4CC(F)(F)CC4C3)cc2)cc1] | N-{4-[(8,8-difluoro-3,6- diazabicyclo[4.3.0]non-3- yl)methyl]phenyl}{[(4- methoxyphenyl)methyl]amino} carboxamide |
| 227 | 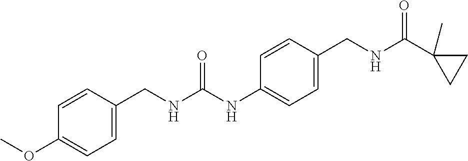 [SMILES: COc1ccc(CNC(=O)Nc2ccc(CNC(=O)C3(C)CC3)cc2)cc1] | {[(4- methoxyphenyl)methyl]amino}- N-(4- {[(methylcyclopropyl) carbonylamino]methyl}phenyl) carboxamide |
| 228 | 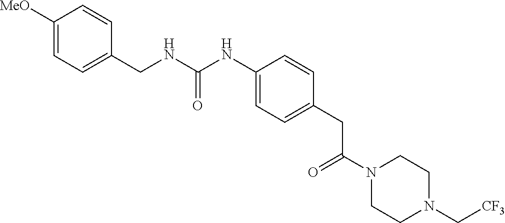 [SMILES: COc1ccc(CNC(=O)Nc2ccc(CC(=O)N3CCN(CC(F)(F)F)CC3)cc2)cc1] | N-[(4- methoxyphenyl)methyl][(4-{2- oxo-2-[4-(2,2,2- trifluoroethyl)piperazinyl]ethyl} phenyl)amino]carboxamide |
| 229 | 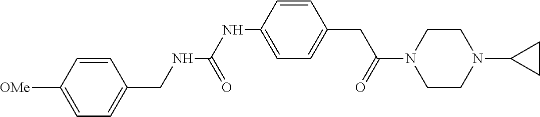 [SMILES: COc1ccc(CNC(=O)Nc2ccc(CC(=O)N3CCN(C4CC4)CC3)cc2)cc1] | ({4-[2-(4- cyclopropylpiperazinyl)-2- oxoethyl]phenyl}amino)-N-[(4- methoxyphenyl)methyl] carboxamide |
| 230 | 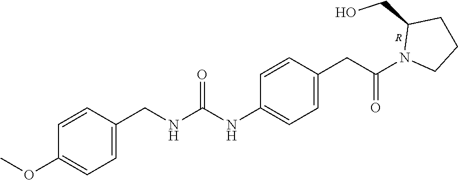 | [(4-{2-[(2R)-2- (hydroxymethyl)pyrrolidinyl]- 2-oxoethyl}phenyl)amino]-N- [(4-methoxyphenyl)methyl] carboxamide |
| 231 | 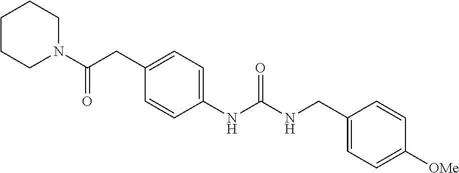 [SMILES: COc1ccc(CNC(=O)Nc2ccc(CC(=O)N3CCCCC3)cc2)cc1] | N-[(4- methoxyphenyl)methyl]{[4-(2- oxo-2- piperidylethyl)phenyl]amino} carboxamide |
| 232 | 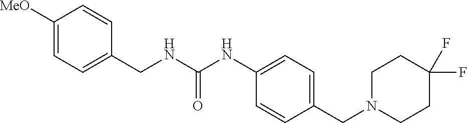 [SMILES: COc1ccc(CNC(=O)Nc2ccc(CN3CCC(F)(F)CC3)cc2)cc1] | ({4-[(4,4- difluoropiperidyl)methyl]phenyl} amino)-N-[(4- methoxyphenyl)methyl] carboxamide |
| 233 | 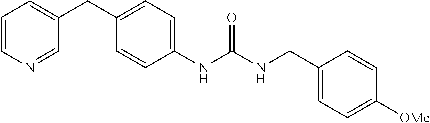 [SMILES: COc1ccc(CNC(=O)Nc2ccc(Cc3cccnc3)cc2)cc1] | N-[(4- methoxyphenyl)methyl]{[4-(3- pyridylmethyl)phenyl]amino} carboxamide |
| 234 | 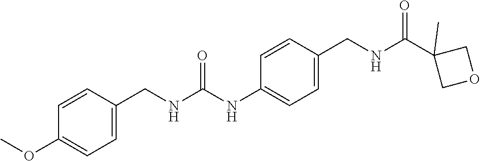 [SMILES: COc1ccc(CNC(=O)Nc2ccc(CNC(=O)C3(C)COC3)cc2)cc1] | {[(4- methoxyphenyl)methyl]amino}- N-(4-{[(3-methyloxetan-3- yl)carbonylamino]methyl} phenyl)carboxamide |
| 235 | 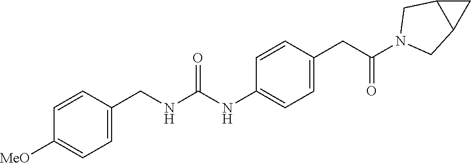 [SMILES: COc1ccc(CNC(=O)Nc2ccc(CC(=O)N3CC4CC4C3)cc2)cc1] | ({4-[2-(3-azabicyclo[3.1.0]hex- 3-yl)-2- oxoethyl]phenyl}amino)-N-[(4- methoxyphenyl)methyl] carboxamide |
| 236 | 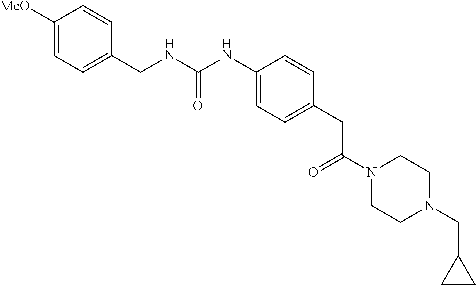 [SMILES: COc1ccc(CNC(=O)Nc2ccc(CC(=O)N3CCN(CC4CC4)CC3)cc2)cc1] | [(4-{2-[4- (cyclopropylmethyl)piperazinyl]- 2-oxoethyl}phenyl)amino]-N- [(4-methoxyphenyl)methyl] carboxamide |
| 237 | 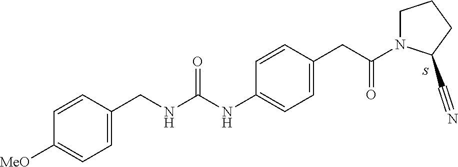 | N-{4-[2-((2S)-2- cyanopyrrolidinyl)-2- oxoethyl]phenyl}{[(4- methoxyphenyl)methyl]amino} carboxamide |
| 238 | 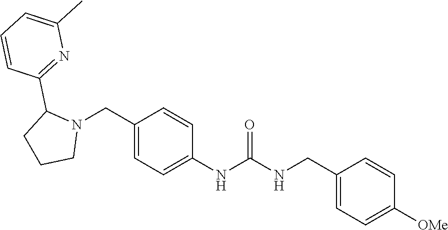 [SMILES: COc1ccc(CNC(=O)Nc2ccc(CN3CCCC3c3cccc(C)n3)cc2)cc1] | {[(4- methoxyphenyl)methyl]amino}- N-(4-{[2-(6-methyl(2- pyridyl))pyrrolidinyl]methyl} phenyl)carboxamide |
| 239 | 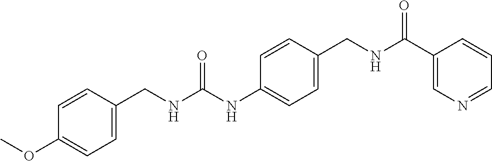 [SMILES: COc1ccc(CNC(=O)Nc2ccc(CNC(=O)c3cccnc3)cc2)cc1] | {[(4- methoxyphenyl)methyl]amino}- N-{4-[(3- pyridylcarbonylamino)methyl] phenyl}carboxamide |
| 240 | 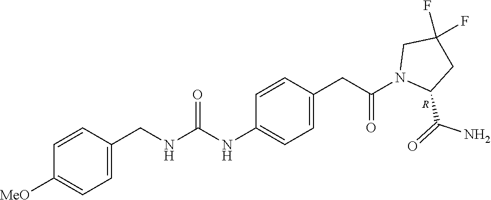 | (2R)-4,4-difluoro-1-{2-[4-({[(4- methoxyphenyl)methyl]amino} carbonylamino)phenyl]acetyl} pyrrolidine-2-carboxamide |
| 241 | 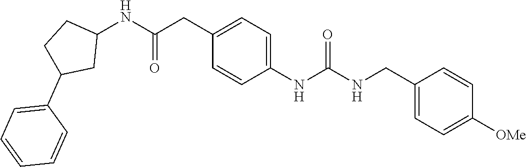 [SMILES: COc1ccc(CNC(=O)Nc2ccc(CC(=O)NC3CCC(c4ccccc4)C3)cc2)cc1] | 2-[4-({N-[(4- methoxyphenyl)methyl] carbamoyl}amino)phenyl]-N-(3- phenylcyclopentyl)acetamide |
| 242 | 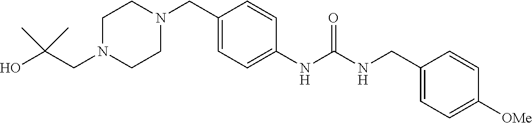 [SMILES: COc1ccc(CNC(=O)Nc2ccc(CN3CCN(CC(C)(C)O)CC3)cc2)cc1] | N-(4-{[4-(2-hydroxy-2- methylpropyl)piperazinyl] methyl}phenyl){[(4- methoxyphenyl)methyl]amino} carboxamide |
| 243 | 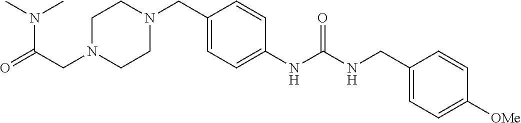 [SMILES: COc1ccc(CNC(=O)Nc2ccc(CN3CCN(CC(=O)N(C)C)CC3)cc2)cc1] | 2-(4-{[4-({[(4- methoxyphenyl)methyl]amino} carbonylamino)phenyl]methyl} piperazinyl)-N,N- dimethylacetamide |
| 244 | 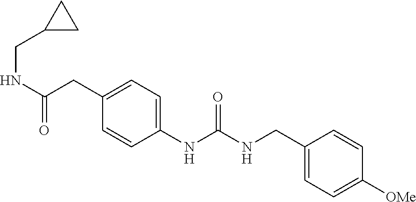 [SMILES: COc1ccc(CNC(=O)Nc2ccc(CC(=O)NCC3CC3)cc2)cc1] | N-(cyclopropylmethyl)-2-[4- ({N-[(4-methoxyphenyl)methyl] carbamoyl} amino)phenyl]acetamide |
| 245 | 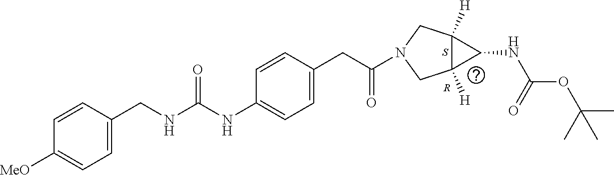 | {[4-(2-{(5S,1R)-6-[(tert- butoxy)carbonylamino]-3- azabicyclo[3.1.0]hex-3-yl}-2- oxoethyl)phenyl]amino}-N-[(4- methoxyphenyl)methyl] carboxamide |
| 246 | 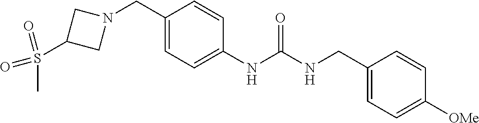 [SMILES: COc1ccc(CNC(=O)Nc2ccc(CN3CC(S(C)(=O)=O)C3)cc2)cc1] | {[(4- methoxyphenyl)methyl]amino}- N-(4-{[3- (methylsulfonyl)azetidinyl] methyl}phenyl)carboxamide |
| 247 | 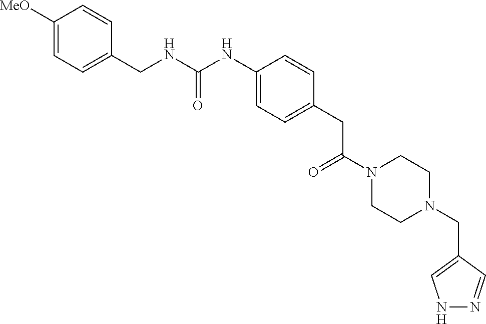 [SMILES: COc1ccc(CNC(=O)Nc2ccc(CC(=O)N3CCN(Cc4cn[nH]c4)CC3)cc2)cc1] | N-[(4- methoxyphenyl)methyl][(4-{2- oxo-2-[4-(pyrazol-4- ylmethyl)piperazinyl]ethyl} phenyl)amino]carboxamide |
| 248 | 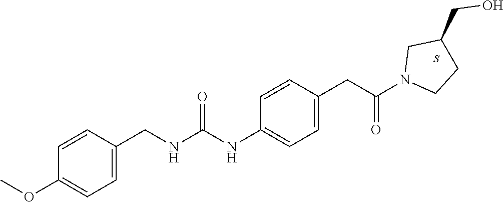 | [(4-{2-[(3S)-3- (hydroxymethyl)pyrrolidinyl]- 2-oxoethyl}phenyl)amino]-N- [(4-methoxyphenyl)methyl] carboxamide |
| 249 | 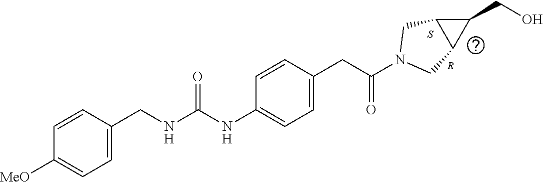 | [(4-{2-[(5S,1R)-6- (hydroxymethyl)-3- azabicyclo[3.1.0]hex-3-yl]-2- oxoethyl}phenyl)amino]-N-[(4- methoxyphenyl)methyl] carboxamide |
| 250 | 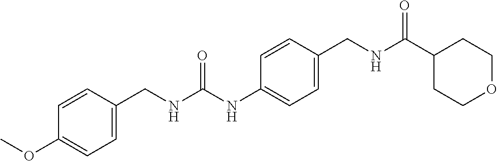 [SMILES: COc1ccc(CNC(=O)Nc2ccc(CNC(=O)C3CCOCC3)cc2)cc1] | {[(4- methoxyphenyl)methyl]amino}- N-{4-[(2H-3,4,5,6- tetrahydropyran-4- ylcarbonylamino)methyl]phenyl} carboxamide |
| 251 | 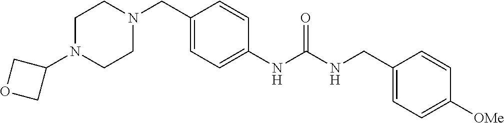 [SMILES: COc1ccc(CNC(=O)Nc2ccc(CN3CCN(C4COC4)CC3)cc2)cc1] | {[(4- methoxyphenyl)methyl]amino}- N-{4-[(4-oxetan-3- ylpiperazinyl)methyl]phenyl} carboxamide |
| 252 | 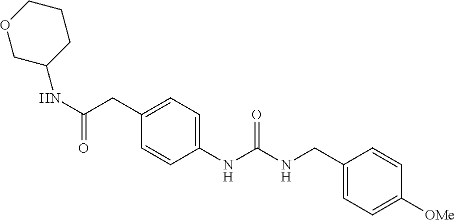 [SMILES: COc1ccc(CNC(=O)Nc2ccc(CC(=O)NC3CCCOC3)cc2)cc1] | N-(2H-3,4,5,6-tetrahydropyran- 3-yl)-2-[4-({N-[(4- methoxyphenyl)methyl]carbamoyl} amino)phenyl]acetamide |
| 253 | 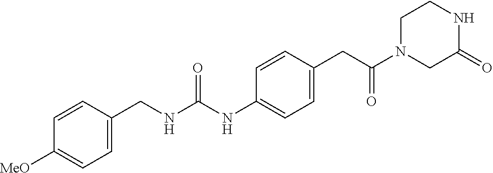 [SMILES: COc1ccc(CNC(=O)Nc2ccc(CC(=O)N3CCNC(=O)C3)cc2)cc1] | {[(4- methoxyphenyl)methyl]amino}- N-{4-[2-oxo-2-(3- oxopiperazinyl)ethyl]phenyl} carboxamide |
| 254 | 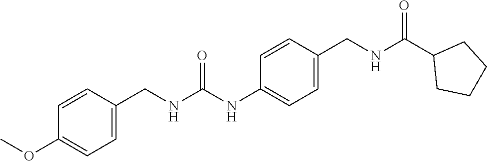 [SMILES: COc1ccc(CNC(=O)Nc2ccc(CNC(=O)C3CCCC3)cc2)cc1] | N-{4- [(cyclopentylcarbonylamino) methyl]phenyl}{[(4- methoxyphenyl)methyl]amino} carboxamide |
| 255 | 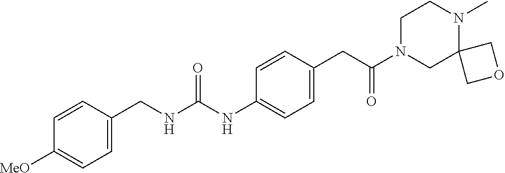 [SMILES: COc1ccc(CNC(=O)Nc2ccc(CC(=O)N3CCN(C)C4(COC4)C3)cc2)cc1] | N-[(4- methoxyphenyl)methyl]({4-[2- (9-methyl-2-oxa-6,9- diazaspiro[3.5]non-6-yl)-2- oxoethyl]phenyl}amino) carboxamide |
| 256 | 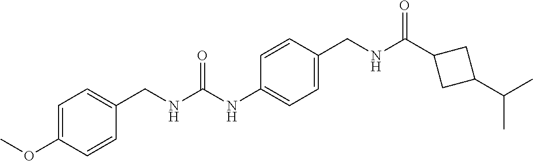 [SMILES: COc1ccc(CNC(=O)Nc2ccc(CNC(=O)C3CC(C(C)C)C3)cc2)cc1] | {[(4- methoxyphenyl)methyl]amino}- N-[4-({[3- (methylethyl)cyclobutyl] carbonylamino}methyl)phenyl] carboxamide |
| 257 | 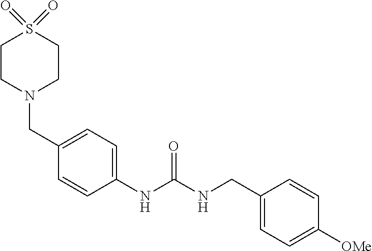 [SMILES: COc1ccc(CNC(=O)Nc2ccc(CN3CCS(=O)(=O)CC3)cc2)cc1] | ({4-[(1,1-dioxo(1,4- thiazaperhydroin-4- yl))methyl]phenyl}amino)-N- [(4-methoxyphenyl)methyl] carboxamide |
| 258 | 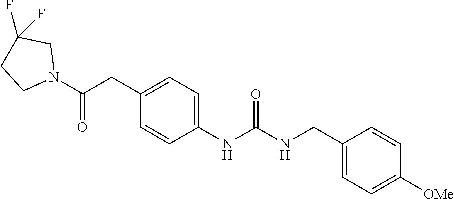 [SMILES: COc1ccc(CNC(=O)Nc2ccc(CC(=O)N3CCC(F)(F)C3)cc2)cc1] | ({4-[2-(3,3- difluoropyrrolidinyl)-2- oxoethyl]phenyl}amino)-N-[(4- methoxyphenyl)methyl] carboxamide |
| 259 | 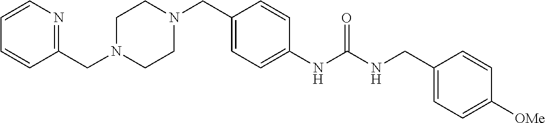 [SMILES: COc1ccc(CNC(=O)Nc2ccc(CN3CCN(Cc4ccccn4)CC3)cc2)cc1] | {[(4- methoxyphenyl)methyl]amino}- N-(4-{[4-(2- pyridylmethyl)piperazinyl]methyl} phenyl)carboxamide |
| 260 | 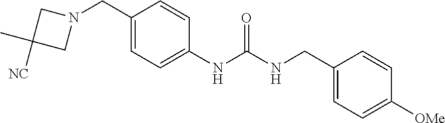 [SMILES: COc1ccc(CNC(=O)Nc2ccc(CN3CC(C)(C#N)C3)cc2)cc1] | N-{4-[(3-cyano-3- methylazetidinyl)methyl]phenyl} {[(4- methoxyphenyl)methyl]amino} carboxamide |
| 261 | 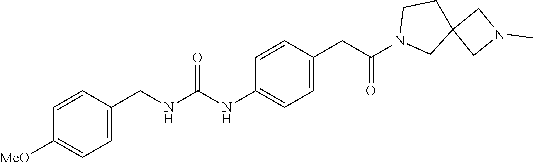 [SMILES: COc1ccc(CNC(=O)Nc2ccc(CC(=O)N3CCC4(CN(C)C4)C3)cc2)cc1] | N-[(4- methoxyphenyl)methyl]({4-[2- (2-methyl-2,6- diazaspiro[3.4]oct-6-yl)-2- oxoethyl]phenyl}amino) carboxamide |
| 262 | 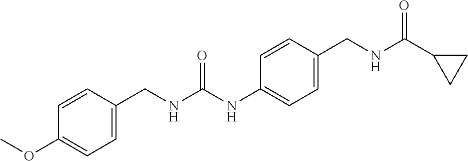 [SMILES: COc1ccc(CNC(=O)Nc2ccc(CNC(=O)C3CC3)cc2)cc1] | N-{4- [(cyclopropylcarbonylamino) methyl]phenyl}{[(4- methoxyphenyl)methyl]amino} carboxamide |
| 263 | 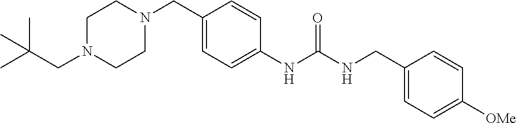 [SMILES: COc1ccc(CNC(=O)Nc2ccc(CN3CCN(CC(C)(C)C)CC3)cc2)cc1] | N-(4-{[4-(2,2- dimethylpropyl)piperazinyl] methyl}phenyl){[(4- methoxyphenyl)methyl]amino} carboxamide |
| 264 | 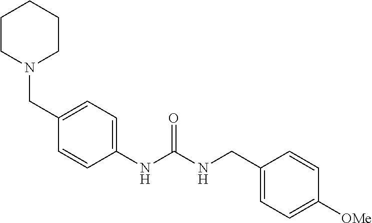 [SMILES: COc1ccc(CNC(=O)Nc2ccc(CN3CCCCC3)cc2)cc1] | N-[(4- methoxyphenyl)methyl]{[4- (piperidylmethyl)phenyl]amino} carboxamide |
| 265 | 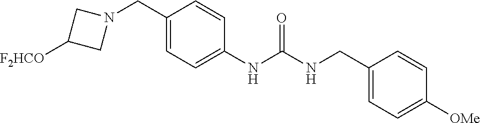 [SMILES: COc1ccc(CNC(=O)Nc2ccc(CN3CC(OC(F)F)C3)cc2)cc1] | N-(4-{[3- (difluoromethoxy)azetidinyl] methyl}phenyl){[(4- methoxyphenyl)methyl]amino} carboxamide |
| 266 | 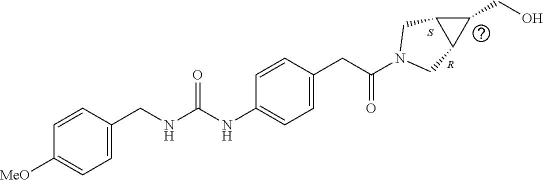 | [(4-{2-[(5S,1R)-6- (hydroxymethyl)-3- azabicyclo[3.1.0]hex-3-yl]-2- oxoethyl}phenyl)amino]-N-[(4- methoxyphenyl)methyl] carboxamide |
| 267 | 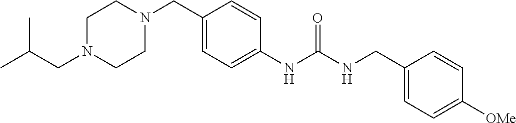 [SMILES: COc1ccc(CNC(=O)Nc2ccc(CN3CCN(CC(C)C)CC3)cc2)cc1] | {[(4- methoxyphenyl)methyl]amino}- N-(4-([4-(2- methylpropyl)piperazinyl]methyl} phenyl)carboxamide |
| 268 | 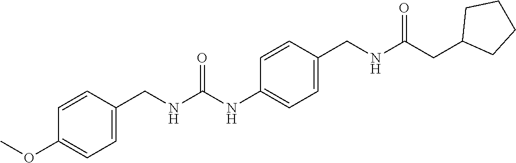 [SMILES: COc1ccc(CNC(=O)Nc2ccc(CNC(=O)CC3CCCC3)cc2)cc1] | 2-cyclopentyl-N-{[4-({[(4- methoxyphenyl)methyl]amino} carbonylamino)phenyl]methyl} acetamide |
| 269 | 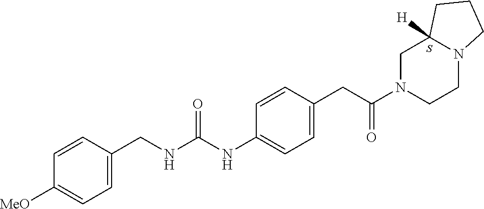 | ({4-[2-((1S)-3,6- diazabicyclo[4.3.0]non-3-yl)-2- oxoethyl]phenyl}amino)-N-[(4- methoxyphenyl)methyl] carboxamide |
| 270 | 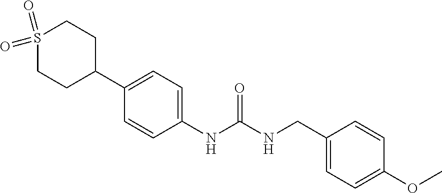 [SMILES: COc1ccc(CNC(=O)Nc2ccc(C3CCS(=O)(=O)CC3)cc2)cc1] | {[4-(1,1-dioxothian-4- yl)phenyl]amino}-N-[(4- methoxyphenyl)methyl] carboxamide |
| 271 | 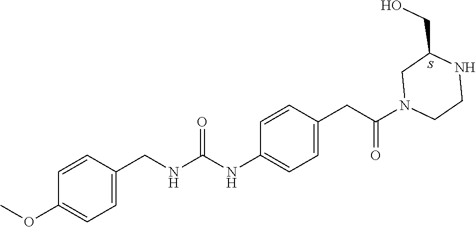 | [(4-{2-[(3S)-3- (hydroxymethyl)piperazinyl]-2- oxoethyl}phenyl)amino]-N-[(4- methoxyphenyl)methyl] carboxamide |
| 272 | 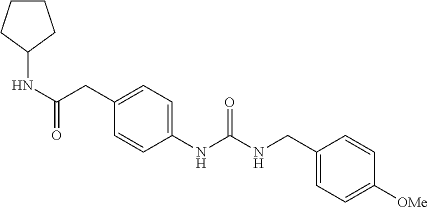 [SMILES: COc1ccc(CNC(=O)Nc2ccc(CC(=O)NC3CCCC3)cc2)cc1] | N-cyclopentyl-2-[4-({N[(4- methoxyphenyl)methyl]carbamoyl} amino)phenyl]acetamide |
| 273 | 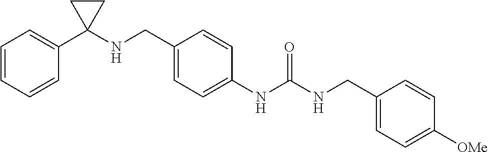 [SMILES: COc1ccc(CNC(=O)Nc2ccc(CNC3(c4ccccc4)CC3)cc2)cc1] | {[(4- methoxyphenyl)methyl]amino}- N-(4-{[(phenylcyclopropyl)amino] methyl}phenyl)carboxamide |
| 274 | 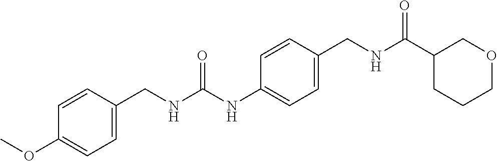 [SMILES: COc1ccc(CNC(=O)Nc2ccc(CNC(=O)C3CCCOC3)cc2)cc1] | {[(4- methoxyphenyl)methyl]amino}- N-{4-[(2H-3,4,5,6- tetrahydropyran-3- ylcarbonylamino)methyl]phenyl} carboxamide |
| 275 | 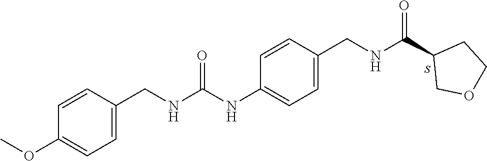 | N-(4-{[((3S)oxolan-3- yl)carbonylamino]methyl} phenyl){[(4- methoxyphenyl)methyl]amino} carboxamide |
| 276 | 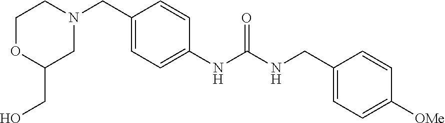 [SMILES: COc1ccc(CNC(=O)Nc2ccc(CN3CCOC(CO)C3)cc2)cc1] | N-(4-{[2- (hydroxymethyl)morpholin-4- yl]methyl}phenyl){[(4- methoxyphenyl)methyl]amino} carboxamide |
| 277 | 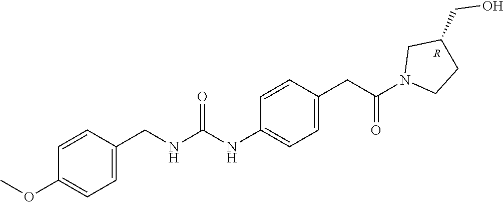 | [(4-{2-[(3R)-3- (hydroxymethyl)pyrrolidinyl]- 2-oxoethyl}phenyl)amino]-N- [(4-methoxyphenyl)methyl] carboxamide |
| 278 | 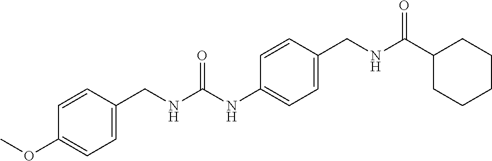 [SMILES: COc1ccc(CNC(=O)Nc2ccc(CNC(=O)C3CCCCC3)cc2)cc1] | N-{4- [(cyclohexylcarbonylamino) methyl]phenyl}{[(4- methoxyphenyl)methyl]amino} carboxamide |
| 279 | 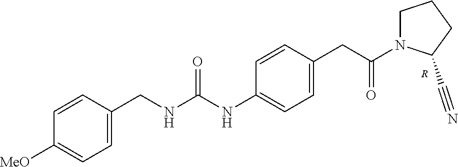 | N-{4-[2-((2R)-2- cyanopyrrolidinyl)-2- oxoethyl]phenyl}{[(4- methoxyphenyl)methyl]amino} carboxamide |
| 280 | 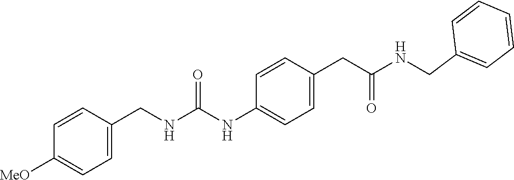 [SMILES: COc1ccc(CNC(=O)Nc2ccc(CC(=O)NCc3ccccc3)cc2)cc1] | 2-[4-({N-[(4- methoxyphenyl)methyl] carbamoyl}amino)phenyl]-N- benzylacetamide |
| 281 | 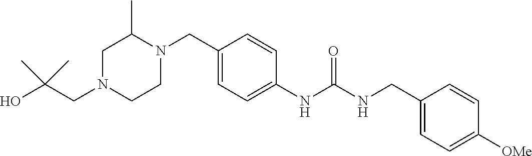 [SMILES: COc1ccc(CNC(=O)Nc2ccc(CN3CCN(CC(C)(C)O)CC3C)cc2)cc1] | N-(4-{[4-(2-hydroxy-2- methylpropyl)-2- methylpiperazinyl]methyl} phenyl){[(4- methoxyphenyl)methyl]amino} carboxamide |
| 282 | 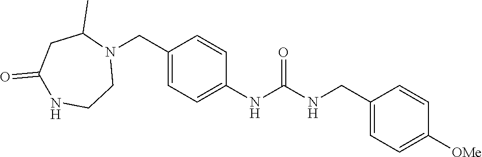 [SMILES: COc1ccc(CNC(=O)Nc2ccc(CN3CCNC(=O)CC3C)cc2)cc1] | {[(4- methoxyphenyl)methyl]amino}- N-{4-[(7-methyl-5-oxo(1,4- diazaperhydroepinyl))methyl] phenyl}carboxamide |
| 283 | 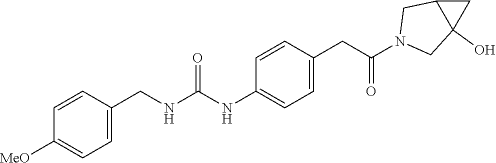 [SMILES: COc1ccc(CNC(=O)Nc2ccc(CC(=O)N3CC4CC4(O)C3)cc2)cc1] | ({4-[2-(1-hydroxy-3- azabicyclo[3.1.0]hex-3-yl)-2- oxoethyl]phenyl}amino)-N-[(4- methoxyphenyl)methyl] carboxamide |
| 284 | 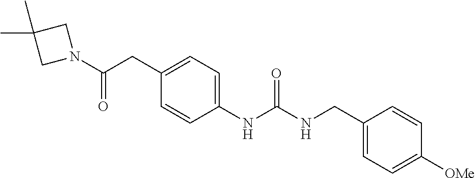 [SMILES: COc1ccc(CNC(=O)Nc2ccc(CC(=O)N3CC(C)(C)C3)cc2)cc1] | ({4-[2-(3,3-dimethylazetidinyl)- 2-oxoethyl]phenyl}amino)-N- [(4-methoxyphenyl)methyl] carboxamide |
| 285 | 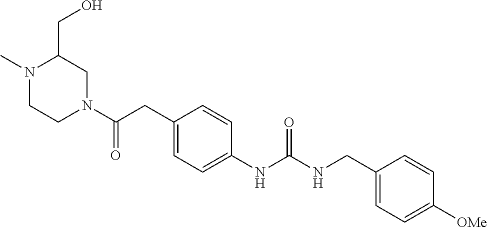 [SMILES: COc1ccc(CNC(=O)Nc2ccc(CC(=O)N3CCN(C)C(CO)C3)cc2)cc1] | N-(4-{2-[3-(hydroxymethyl)-4- methylpiperazinyl]-2- oxoethyl}phenyl){[(4- methoxyphenyl)methyl]amino} carboxamide |
| 286 | 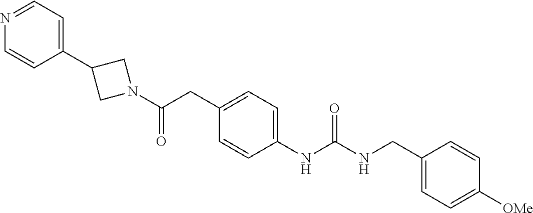 [SMILES: COc1ccc(CNC(=O)Nc2ccc(CC(=O)N3CC(c4ccncc4)C3)cc2)cc1] | N-[(4- methoxyphenyl)methyl]({4-[2- oxo-2-(3-(4- pyridyl)azetidinyl)ethyl]phenyl} amino)carboxamide |
| 287 | 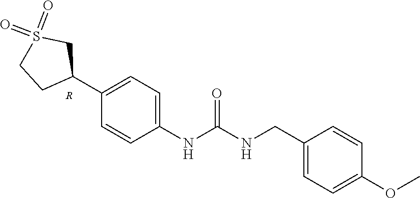 | {[4-((3R)-1,1-dioxothiolan-3- yl)phenyl]amino}-N-[(4- methoxyphenyl)methyl] carboxamide |
| 288 | 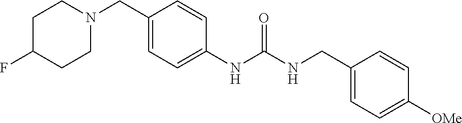 [SMILES: COc1ccc(CNC(=O)Nc2ccc(CN3CCC(F)CC3)cc2)cc1] | N-{4-[(4- fluoropiperidyl)methyl]phenyl} {[(4- methoxyphenyl)methyl]amino} carboxamide |
| 289 | 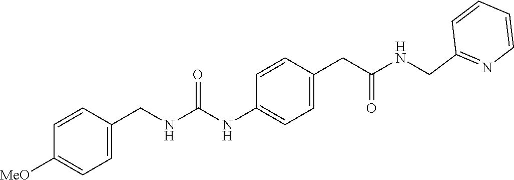 [SMILES: COc1ccc(CNC(=O)Nc2ccc(CC(=O)NCc3ccccn3)cc2)cc1] | 2-[4-({N-[(4- methoxyphenyl)methyl]carbamoyl} amino)phenyl]-N-(2- pyridylmethyl)acetamide |
| 290 | 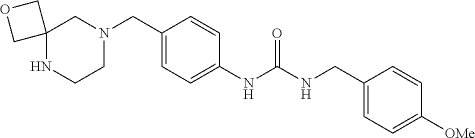 [SMILES: COc1ccc(CNC(=O)Nc2ccc(CN3CCNC4(COC4)C3)cc2)cc1] | {[(4- methoxyphenyl)methyl]amino}- N-{4-[(2-oxa-6,9- diazaspiro[3.5]non-6- yl)methyl]phenyl}carboxamide |
| 291 | 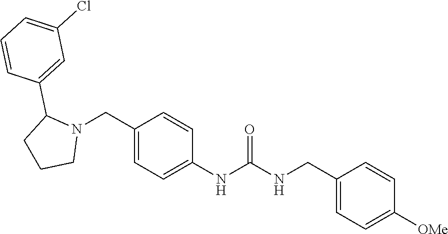 [SMILES: COc1ccc(CNC(=O)Nc2ccc(CN3CCCC3c3cccc(Cl)c3)cc2)cc1] | N-(4-{[2-(3- chlorophenyl)pyrrolidinyl] methyl}phenyl){[(4- methoxyphenyl)methyl]amino} carboxamide |
| 292 | 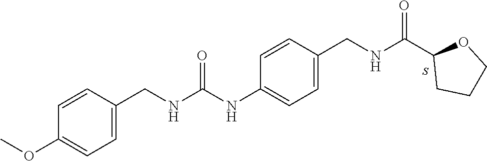 | N-(4-{[((2S)oxolan-2- yl)carbonylamino]methyl} phenyl){[(4- methoxyphenyl)methyl]amino} carboxamide |
| 293 | 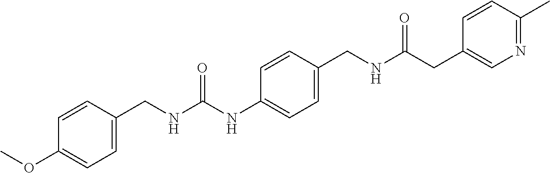 | N-{[4-({[(4- methoxyphenyl)methyl]amino} carbonylamino)phenyl]methyl}- 2-(6-methyl(3- pyridyl))acetamide |
| 294 | 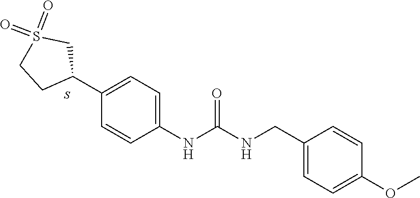 | {[4-((3S)-1,1-dioxothiolan-3- yl)phenyl]amino}-N-[(4- methoxyphenyl)methyl] carboxamide |
| 295 | 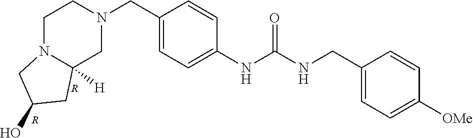 | N-{4-[((1R,8R)-8-hydroxy-3,6- diazabicyclo[4.3.0]non-3- yl)methyl]phenyl}{[(4- methoxyphenyl)methyl]amino} carboxamide |
| 296 | 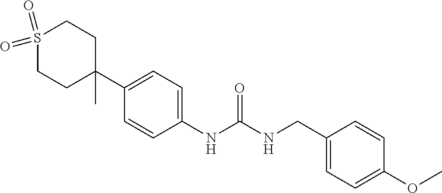 [SMILES: COc1ccc(CNC(=O)Nc2ccc(C3(C)CCS(=O)(=O)CC3)cc2)cc1] | N-[(4- methoxyphenyl)methyl]{[4-(4- methyl-1,1-dioxothian-4- yl)phenyl]amino}carboxamide |
| 297 | 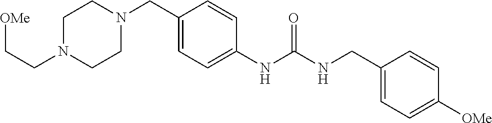 [SMILES: COCCN1CCN(Cc2ccc(NC(=O)NCc3ccc(OC)cc3)cc2)CC1] | N-(4-{[4-(2- methoxyethyl)piperazinyl] methyl}phenyl){[(4- methoxyphenyl)methyl]amino} carboxamide |
| 298 | 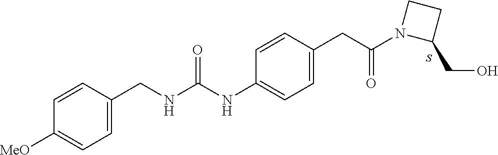 | [(4-{2-[(2S)-2- (hydroxymethyl)azetidinyl]-2- oxoethyl}phenyl)amino]-N-[(4- methoxyphenyl)methyl] carboxamide |
| 299 | 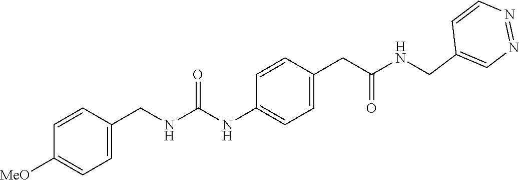 [SMILES: COc1ccc(CNC(=O)Nc2ccc(CC(=O)NCc3ccnnc3)cc2)cc1] | 2-[4-({N-[(4- methoxyphenyl)methyl] carbamoyl}amino)phenyl]-N- (pyridazin-4- ylmethyl)acetamide |
| 300 | 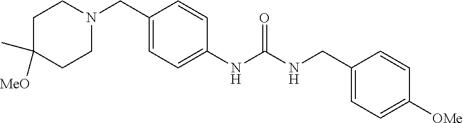 [SMILES: COc1ccc(CNC(=O)Nc2ccc(CN3CCC(C)(OC)CC3)cc2)cc1] | N-{4-[(4-methoxy-4- methylpiperidyl)methyl]phenyl} {[(4- methoxyphenyl)methyl]amino} carboxamide |
| 301 | 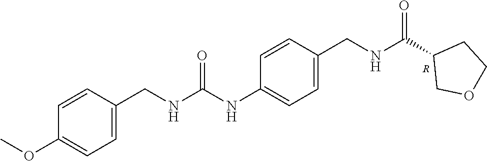 | N-(4-{[((3R)oxolan-3- yl)carbonylamino]methyl} phenyl){[(4- methoxyphenyl)methyl]amino} carboxamide |
| 302 | 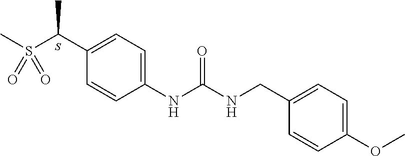 | N-{4-[(1S)-1- (methylsulfonyl)ethyl]phenyl}{ [(4- methoxyphenyl)methyl]amino} carboxamide |
| 303 | 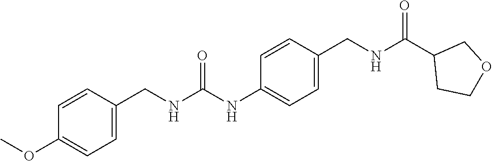 [SMILES: COc1ccc(CNC(=O)Nc2ccc(CNC(=O)C3CCOC3)cc2)cc1] | {[(4- methoxyphenyl)methyl]amino}- N-{4-[(oxolan-3- ylcarbonylamino)methyl]phenyl} carboxamide |
| 304 | 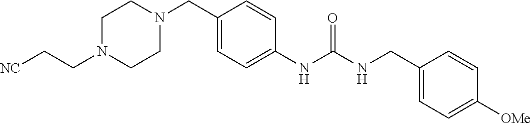 [SMILES: COc1ccc(CNC(=O)Nc2ccc(CN3CCN(CCC#N)CC3)cc2)cc1] | N-(4-{[4-(2- cyanoethyl)piperazinyl]methyl} phenyl){[(4- methoxyphenyl)methyl]amino} carboxamide |
| 305 | 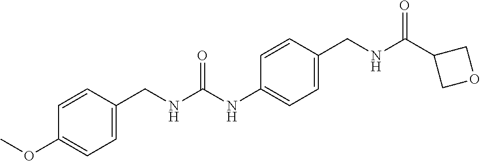 [SMILES: COc1ccc(CNC(=O)Nc2ccc(CNC(=O)C3COC3)cc2)cc1] | {[(4- methoxyphenyl)methyl]amino}- N-{4-[(oxetan-3- ylcarbonylamino)methyl]phenyl} carboxamide |
| 306 | 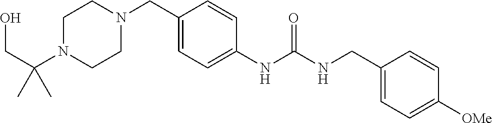 [SMILES: COc1ccc(CNC(=O)Nc2ccc(CN3CCN(C(C)(C)CO)CC3)cc2)cc1] | N-(4-{[4-(2-hydroxy-tert- butyl)piperazinyl]methyl}phenyl) {[(4- methoxyphenyl)methyl]amino} carboxamide |
| 307 | 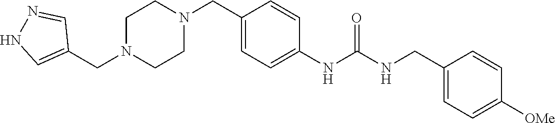 [SMILES: COc1ccc(CNC(=O)Nc2ccc(CN3CCN(Cc4cn[nH]c4)CC3)cc2)cc1] | {[(4- methoxyphenyl)methyl]amino}- N-(4-{[4-(pyrazol-4- ylmethyl)piperazinyl]methyl} phenyl)carboxamide |
| 308 | 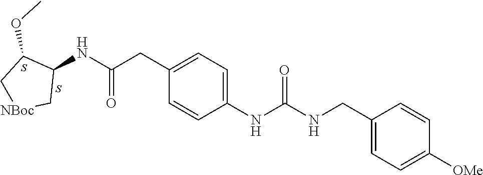 | tert-butyl (3S,4S)-3-methoxy-4- {2-[4-({N-[(4- methoxyphenyl)methyl]carbamoyl} amino)phenyl]acetylamino} pyrrolidinecarboxylate |
| 309 | 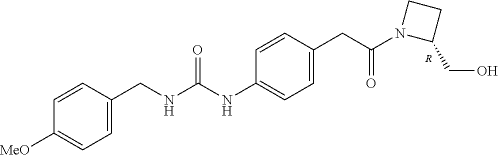 | [(4-{2-[(2R)-2- (hydroxymethyl)azetidinyl]-2- oxoethyl}phenyl)amino]-N-[(4- methoxyphenyl)methyl] carboxamide |
| 310 | 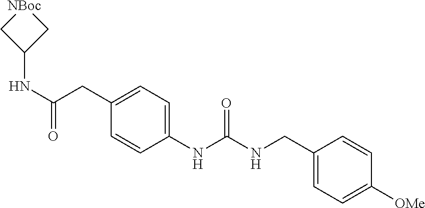 [SMILES: COc1ccc(CNC(=O)Nc2ccc(CC(=O)NC3CN(C(=O)OC(C)(C)C)C3)cc2)cc1] | tert-butyl 3-{2-[4-({N-[(4- methoxyphenyl)methyl]carbamoyl} amino)phenyl]acetylamino} azetidinecarboxylate |
| 311 | 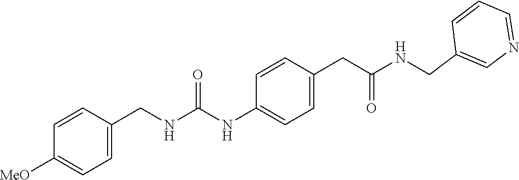 [SMILES: COc1ccc(CNC(=O)Nc2ccc(CC(=O)NCc3cccnc3)cc2)cc1] | 2-[4-({N-[(4- methoxyphenyl)methyl] carbamoyl}amino)phenyl]-N-(3- pyridylmethyl)acetamide |
| 312 | 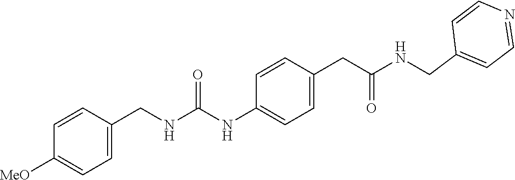 [SMILES: COc1ccc(CNC(=O)Nc2ccc(CC(=O)NCc3ccncc3)cc2)cc1] | 2-[4-({N-[(4- methoxyphenyl)methyl]carbam oyl}amino)phenyl]-N-(4- pyridylmethyl)acetamide |
| 313 | 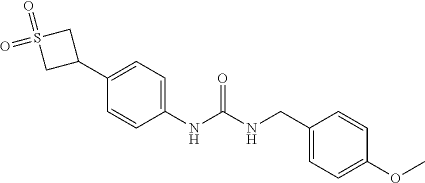 [SMILES: COc1ccc(CNC(=O)Nc2ccc(C3CS(=O)(=O)C3)cc2)cc1] | {[4-(1,1-dioxothietan-3- yl)phenyl]amino}-N-[(4- methoxyphenyl)methyl] carboxamide |
| 314 | 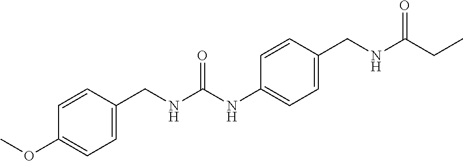 [SMILES: CCC(=O)NCc1ccc(NC(=O)NCc2ccc(OC)cc2)cc1] | N-{[4-({[(4- methoxyphenyl)methyl]amino} carbonylamino)phenyl]methyl} propanamide |
| 315 | 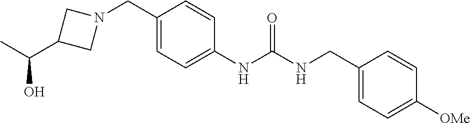 [SMILES: COc1ccc(CNC(=O)Nc2ccc(CN3CC([C@H](C)O)C3)cc2)cc1] | N-(4-{[3-((1S)-1- hydroxyethyl)azetidinyl]methyl} phenyl){[(4- methoxyphenyl)methyl]amino} carboxamide |
| 316 | 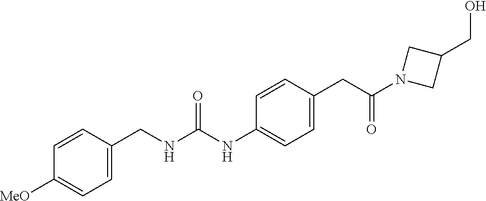 [SMILES: COc1ccc(CNC(=O)Nc2ccc(CC(=O)N3CC(CO)C3)cc2)cc1] | N-(4-{2-[3- (hydroxymethyl)azetidinyl]-2- oxoethyl}phenyl){[(4- methoxyphenyl)methyl]amino} carboxamide |
| 317 | 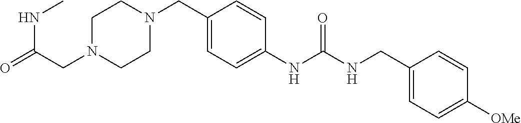 [SMILES: CNC(=O)CN1CCN(Cc2ccc(NC(=O)NCc3ccc(OC)cc3)cc2)CC1] | 2-(4-{[4-({[(4- methoxyphenyl)methyl]amino} carbonylamino)phenyl]methyl} piperazinyl)-N- methylacetamide |
| 318 | 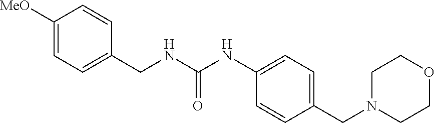 [SMILES: COc1ccc(CNC(=O)Nc2ccc(CN3CCOCC3)cc2)cc1] | N-[(4- methoxyphenyl)methyl]{[4- (morpholin-4- ylmethyl)phenyl]amino} carboxamide |
| 319 | 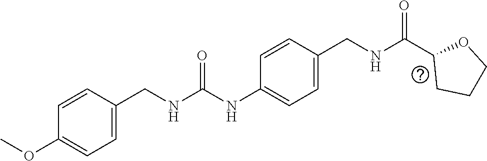 [SMILES: COc1ccc(CNC(=O)Nc2ccc(CNC(=O)[C@H]3CCCO3)cc2)cc1] | N-(4-{[((2R)oxolan-2- yl)carbonylamino]methyl} phenyl){[(4- methoxyphenyl)methyl]amino} carboxamide |
| 320 | 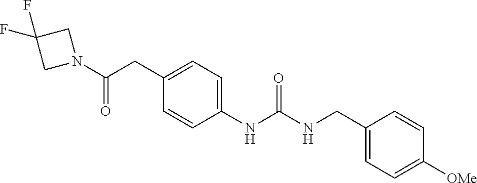 [SMILES: COc1ccc(CNC(=O)Nc2ccc(CC(=O)N3CC(F)(F)C3)cc2)cc1] | ({4-[2-(3,3-difluoroazetidinyl)- 2-oxoethyl]phenyl}amino)-N- [(4-rnethoxyphenyl)methyl] carboxamide |
| 321 | 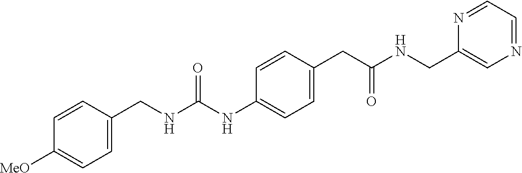 [SMILES: COc1ccc(CNC(=O)Nc2ccc(CC(=O)NCc3cnccn3)cc2)cc1] | 2-[4-({N-[(4- methoxyphenyl)methyl]carbamoyl} amino)phenyl]-N-(pyrazin- 2-ylmethyl)acetamide |
| 322 | 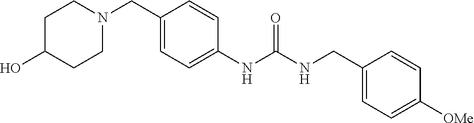 [SMILES: COc1ccc(CNC(=O)Nc2ccc(CN3CCC(O)CC3)cc2)cc1] | N-{4-[(4- hydroxypiperidyl)methyl]phenyl} {[(4- methoxyphenyl)methyl]amino} carboxamide |
| 323 | 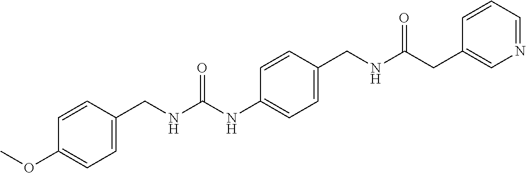 [SMILES: COc1ccc(CNC(=O)Nc2ccc(CNC(=O)Cc3cccnc3)cc2)cc1] | N-{[4-({[(4- methoxyphenyl)methyl]amino} carbonylamino)phenyl]methyl}- 2-(3-pyridyl)acetamide |
| 324 | 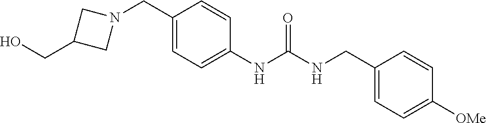 [SMILES: COc1ccc(CNC(=O)Nc2ccc(CN3CC(CO)C3)cc2)cc1] | N-(4-{[3- (hydroxymethyl)azetidinyl]methyl} phenyl){[(4- methoxyphenyl)methyl]amino} carboxamide |
| 325 | 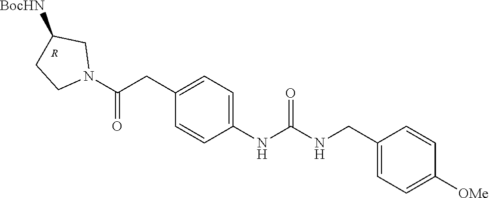 | ([4-(2-{(3R)-3-[(tert- butoxy)carbonylamino] pyrrolidinyl}-2- oxoethyl)phenyl]amino}-N-[(4- methoxyphenyl)methyl] carboxamide |
| 326 | 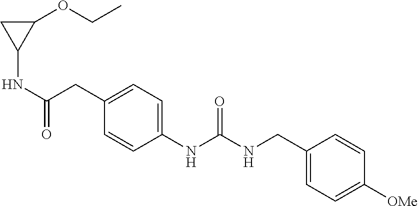 [SMILES: CCOC1CC1NC(=O)Cc1ccc(NC(=O)NCc2ccc(OC)cc2)cc1] | N-(2-ethoxycyclopropyl)-2-[4- ({N-[(4- methoxyphenyl)methyl]carbamoyl} amino)phenyl]acetamide |
| 327 | 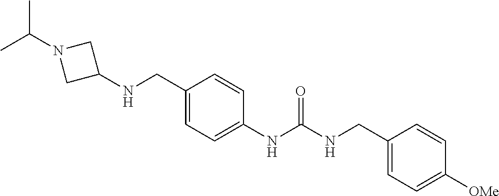 [SMILES: COc1ccc(CNC(=O)Nc2ccc(CNC3CN(C(C)C)C3)cc2)cc1] | {[(4- methoxyphenyl)methyl]amino}- N-[4-({[1- (methylethyl)azetidin-3- yl]amino}methyl)phenyl] carboxamide |
| 328 | 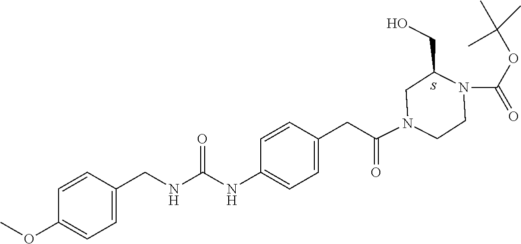 | tert-butyl (2S)-2- (hydroxymethyl)-4-{2-[4-({N- [(4-methoxyphenyl)methyl] carbamoyl} amino)phenyl]acetyl} piperazinecarboxylate |
| 329 | 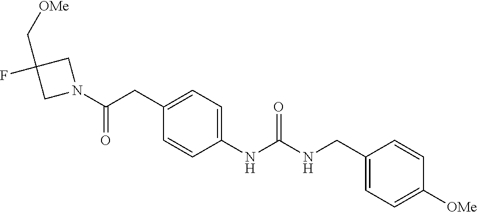 [SMILES: COCC1(F)CN(C(=O)Cc2ccc(NC(=O)NCc3ccc(OC)cc3)cc2)C1] | [(4-(2-[3-fluoro-3- (hydroxymethyl)azetidinyl]-2- oxoethyl}phenyl)amino]-N-[(4- methoxyphenyl)methyl] carboxamide |
| 330 | 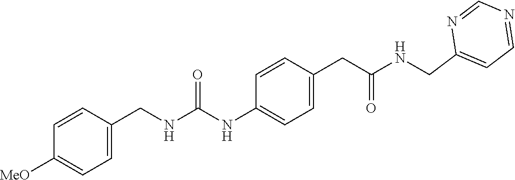 [SMILES: COc1ccc(CNC(=O)Nc2ccc(CC(=O)NCc3ccncn3)cc2)cc1] | 2-[4-({N-[(4- methoxyphenyl)methyl] carbamoyl}amino)phenyl]-N- (pyrimidin-4- ylmethyl)acetamide |
| 331 | 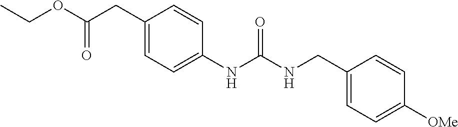 [SMILES: CCOC(=O)Cc1ccc(NC(=O)NCc2ccc(OC)cc2)cc1] | ethyl 2-[4-({[(4- methoxyphenyl)methyl]amino} carbonylamino)phenyl]acetate |
| 332 | 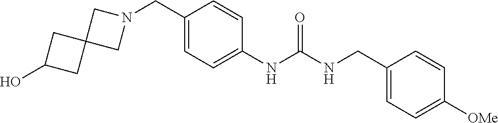 [SMILES: COc1ccc(CNC(=O)Nc2ccc(CN3CC4(CC(O)C4)C3)cc2)cc1] | N-{4-[(6-hydroxy-2- azaspiro[3.3]hept-2- yl)methyl]phenyl}{[(4- methoxyphenyl)methyl]amino} carboxamide |
| 333 | 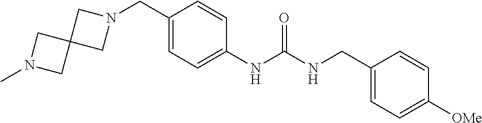 [SMILES: COc1ccc(CNC(=O)Nc2ccc(CN3CC4(CN(C)C4)C3)cc2)cc1] | N-{4-[(6-hydroxy-2- azaspiro[3.3]hept-2- yl)methyl]phenyl}{[(4- methoxyphenyl)methyl]amino} carboxamide |
| 334 | 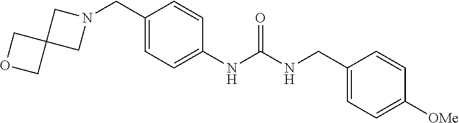 [SMILES: COc1ccc(CNC(=O)Nc2ccc(CN3CC4(COC4)C3)cc2)cc1] | {[(4- methoxyphenyl)methyl]amino}- N-{4-[(6-oxa-2- azaspiro[3.3]hept-2- yl)methyl]phenyl}carboxamide |
| 335 | 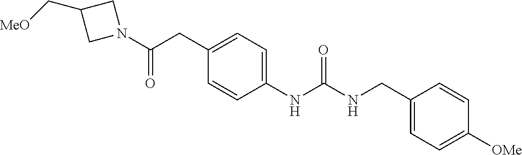 [SMILES: COCC1CN(C(=O)Cc2ccc(NC(=O)NCc3ccc(OC)cc3)cc2)C1] | [(4-{2-[3- (methoxymethyl)azetidinyl]-2- oxoethyl}phenyl)amino]-N-[(4- methoxyphenyl)methyl] carboxamide |
| 336 | 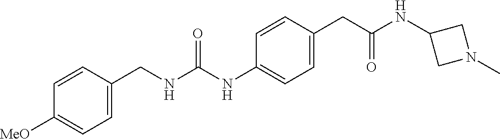 [SMILES: COc1ccc(CNC(=O)Nc2ccc(CC(=O)NC3CN(C)C3)cc2)cc1] | 2-[4-({[(4- methoxyphenyl)methyl]amino} carbonylamino)phenyl]-N-(1- methylazetidin-3-yl)acetamide |
| 337 | 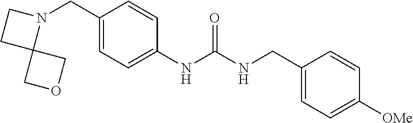 [SMILES: COc1ccc(CNC(=O)Nc2ccc(CN3CCC34COC4)cc2)cc1] | {[(4- methoxyphenyl)methyl]amino}- N-{4-[(6-oxa-1- azaspiro[3.3]heptyl)methyl] phenyl}carboxamide |
| 338 | 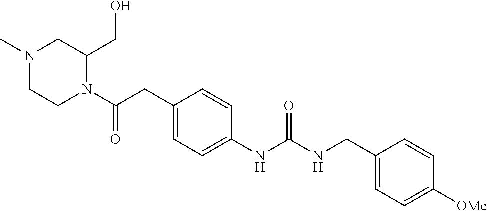 [SMILES: COc1ccc(CNC(=O)Nc2ccc(CC(=O)N3CCN(C)CC3CO)cc2)cc1] | N-(4-{2-[2-(hydroxymethyl)-4- methylpiperazinyl]-2- oxoethyl}phenyl){[(4- methoxyphenyl)methyl]amino} carboxamide |
| 339 | 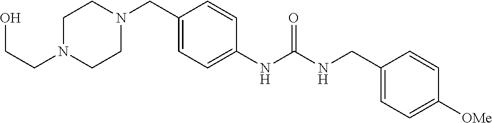 [SMILES: COc1ccc(CNC(=O)Nc2ccc(CN3CCN(CCO)CC3)cc2)cc1] | N-(4-{[4-(2- hydroxyethyl)piperazinyl] methyl}phenyl){[(4- methoxyphenyl)methyl]amino} carboxamide |
| 340 | 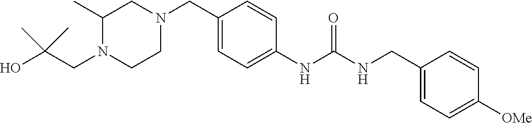 [SMILES: COc1ccc(CNC(=O)Nc2ccc(CN3CCN(CC(C)(C)O)C(C)C3)cc2)cc1] | N-(4-{[4-(2-hydroxy-2- methylpropyl)-3- methylpiperazinyl]methyl} phenyl){[(4- methoxyphenyl)methyl]amino} carboxamide |
| 341 | 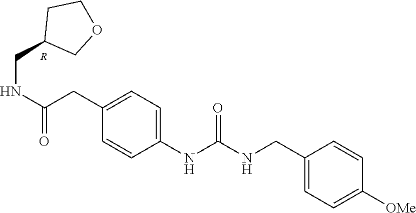 | N-[((3R)oxolan-3-yl)methyl]-2- [4-({N-[(4- methoxyphenyl)methyl]carbamoyl} amino)phenyl]acetamide |
| 342 | 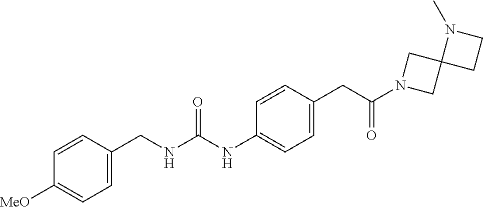 [SMILES: COc1ccc(CNC(=O)Nc2ccc(CC(=O)N3CC4(CCN4C)C3)cc2)cc1] | N-[(4- methoxyphenyl)methyl]({4-[2- (5-methyl-2,5- diazaspiro[3.3]hept-2-yl)-2- oxoethyl]phenyl}amino) carboxamide |
| 343 | 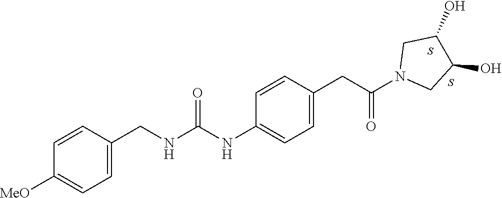 | ({4-[2-((3S,4S)-3,4- dihydroxypyrrolidinyl)-2- oxoethyl]phenyl}amino)-N-[(4- methoxyphenyl)methyl] carboxamide |
| 344 | 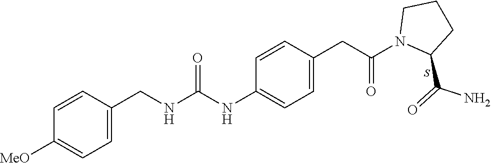 | (2S)-1-{2-[4-({[(4- methoxyphenyl)methyl]amino} carbonylamino)phenyl]acetyl} pyrrolidine-2-carboxamide |
| 345 | 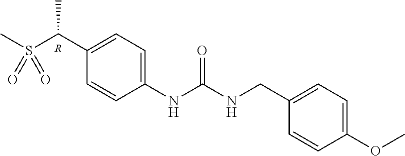 | N-{4-[(1R)-1- (methylsulfonyl)ethyl]phenyl} {[(4- methoxyphenyl)methyl]amino} carboxamide |
| 346 | 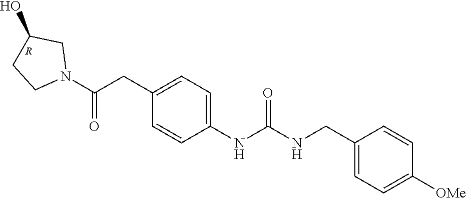 | ({4-[2-((3R)-3- hydroxypyrrolidinyl)-2- oxoethyl]phenyl}amino)-N-[(4- methoxyphenyl)methyl] carboxamide |
| 347 | 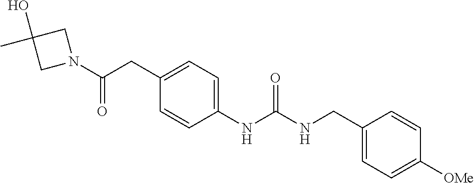 [SMILES: COc1ccc(CNC(=O)Nc2ccc(CC(=O)N3CC(C)(O)C3)cc2)cc1] | ({4-[2-(3-hydroxy-3- methylazetidinyl)-2- oxoethyl]phenyl}amino)-N-[(4- methoxyphenyl)methyl] carboxamide |
| 348 | 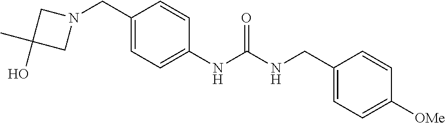 [SMILES: COc1ccc(CNC(=O)Nc2ccc(CN3CC(C)(O)C3)cc2)cc1] | N-{4-[(3-hydroxy-3- methylazetidinyl)methyl]phenyl} {[(4- methoxyphenyl)methyl]amino} carboxamide |
| 349 | 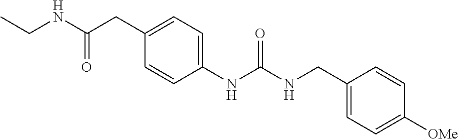 [SMILES: CCNC(=O)Cc1ccc(NC(=O)NCc2ccc(OC)cc2)cc1] | N-ethyl-2-[4-((N-[(4- methoxyphenyl)methyl]carbamoyl} amino)phenyl]acetamide |
| 350 | 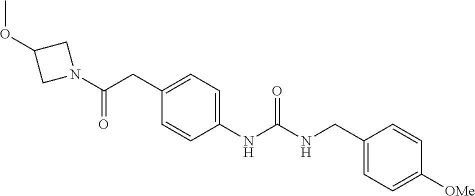 [SMILES: COc1ccc(CNC(=O)Nc2ccc(CC(=O)N3CC(OC)C3)cc2)cc1] | ({4-[2-(3-methoxyazetidinyl)-2- oxoethyl]phenyl}amino)-N-[(4- methoxyphenyl)methyl] carboxamide |
| 351 | 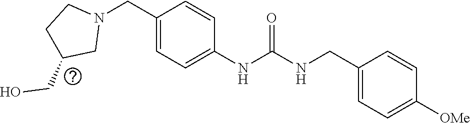 [SMILES: COc1ccc(CNC(=O)Nc2ccc(CN3CC[C@@H](CO)C3)cc2)cc1] | N-(4-{[(3R)-3- (hydroxymethyl)pyrrolidinyl] methyl}phenyl){[(4- methoxyphenyl)methyl]amino} carboxamide |
| 352 | 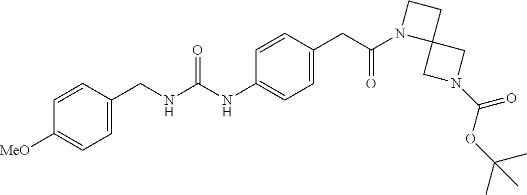 [SMILES: COc1ccc(CNC(=O)Nc2ccc(CC(=O)N3CCC34CN(C(=O)OC(C)(C)C)C4)cc2)cc1] | tert-butyl 5-(2-[4-({[(4- methoxyphenyl)methyl]amino} carbonylamino)phenyl]acetyl}- 2,5-diazaspiro[3.3]heptane-2- carboxylate |
| 353 | 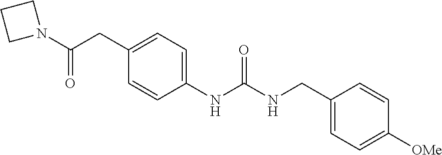 [SMILES: COc1ccc(CNC(=O)Nc2ccc(CC(=O)N3CCC3)cc2)cc1] | {[4-(2-azetidinyl-2- oxoethyl)phenyl]amino}-N-[(4- methoxyphenyl)methyl] carboxamide |
| 354 | 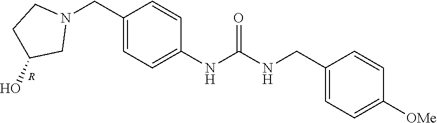 | ({4-[((3R)-3- hydroxypyrrolidinyl)methyl] phenyl}amino)-N-[(4- methoxyphenyl)methyl] carboxamide |
| 355 | 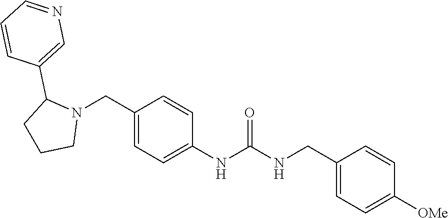 [SMILES: COc1ccc(CNC(=O)Nc2ccc(CN3CCCC3c3cccnc3)cc2)cc1] | 1-(4-methoxybenzyl)-3-(4-((2- (pyridin-3-yl)pyrrolidin-1- yl)methyl)phenyl)urea |
| 356 | 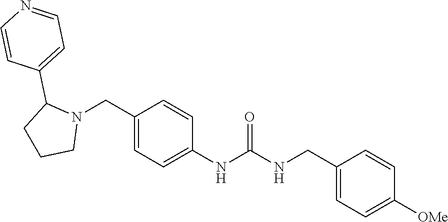 [SMILES: COc1ccc(CNC(=O)Nc2ccc(CN3CCCC3c3ccncc3)cc2)cc1] | 1-(4-methoxybenzyl)-3-(4-((2- (pyridin-4-yl)pyrrolidin-1- yl)methyl)phenyl)urea |
| 357 | 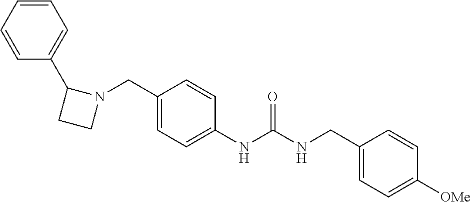 [SMILES: COc1ccc(CNC(=O)Nc2ccc(CN3CCC3c3ccccc3)cc2)cc1] | 1-(4-methoxybenzyl)-3-(4-((2- phenylazetidin-1- yl)methyl)phenyl)urea |
| 358 | 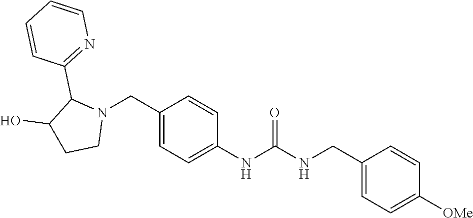 [SMILES: COc1ccc(CNC(=O)Nc2ccc(CN3CCC(O)C3c3ccccn3)cc2)cc1] | 1-(4-((3-hydroxy-2-(pyridin-2- yl)pyrrolidin-1- yl)methyl)phenyl)-3-(4- methoxybenzyl)urea |
| 359 | 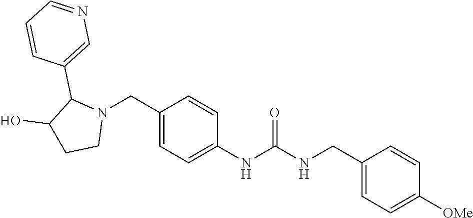 [SMILES: COc1ccc(CNC(=O)Nc2ccc(CN3CCC(O)C3c3cccnc3)cc2)cc1] | 1-(4-((3-hydroxy-2-(pyridin-3- yl)pyrrolidin-1- yl)methyl)phenyl)-3-(4- methoxybenzyl)urea |
| 360 | 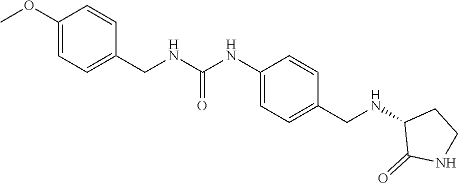 [SMILES: COc1ccc(CNC(=O)Nc2ccc(CN[C@@H]3CCNC3=O)cc2)cc1] | (R)-1-(4-methoxybenzyl)-3-(4- (((2-oxopyrrolidin-3- yl)amino)methyl)phenyl)urea |
| 361 | 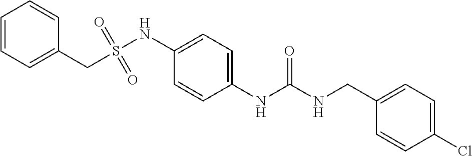 [SMILES: O=C(NCc1ccc(Cl)cc1)Nc1ccc(NS(=O)(=O)Cc2ccccc2)cc1] | N-(4-(3-(4- chlorobenzyl)ureido)phenyl)-1- phenylmethanesulfonamide |
| 362 | 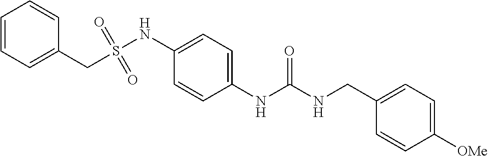 [SMILES: COc1ccc(CNC(=O)Nc2ccc(NS(=O)(=O)Cc3ccccc3)cc2)cc1] | N-(4-(3-(4- methoxybenzyl)ureido)phenyl)- 1-phenylmethanesulfonamide |
| 363 | 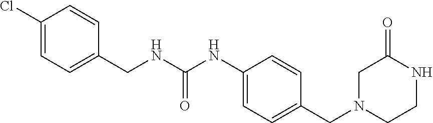 [SMILES: O=C1CN(Cc2ccc(NC(=O)NCc3ccc(Cl)cc3)cc2)CCN1] | 1-(4-chlorobenzyl)-3-(4-((3- oxopiperazin-1- yl)methyl)phenyl)urea |
| 364 | 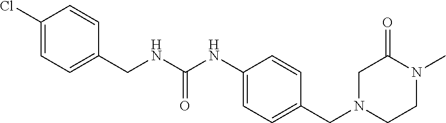 [SMILES: CN1CCN(Cc2ccc(NC(=O)NCc3ccc(Cl)cc3)cc2)CC1=O] | 1-(4-chlorobenzyl)-3-(4-((4- methyl-3-oxopiperazin-1- yl)methyl)phenyl)urea |
| 365 | 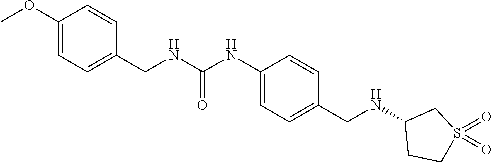 [SMILES: COc1ccc(CNC(=O)Nc2ccc(CN[C@H]3CCS(=O)(=O)C3)cc2)cc1] | (S)-1-(4-(((1,1- dioxidotetrahydrothiophen-3- yl)amino)methyl)phenyl)-3-(4- methoxybenzyl)urea |
| 366 | 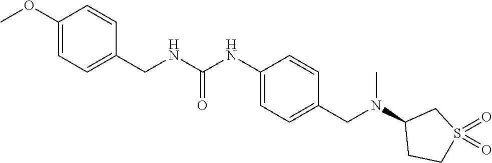 [SMILES: COc1ccc(CNC(=O)Nc2ccc(CN(C)[C@@H]3CCS(=O)(=O)C3)cc2)cc1] | (R)-1-(4-(((1,1- dioxidotetrahydrothiophen-3- yl)(methyl)amino)methyl)phenyl)- 3-(4-methoxybenzyl)urea |
| 367 | 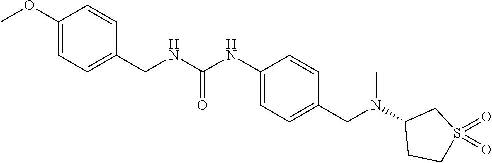 [SMILES: COc1ccc(CNC(=O)Nc2ccc(CN(C)[C@H]3CCS(=O)(=O)C3)cc2)cc1] | (S)-1-(4-(((1,1- dioxidotetrahydrothiophen-3- yl)(methyl)amino)methyl)phenyl)- 3-(4-methoxybenzyl)urea |
| 368 | 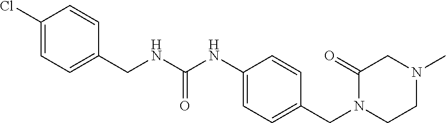 [SMILES: CN1CCN(Cc2ccc(NC(=O)NCc3ccc(Cl)cc3)cc2)C(=O)C1] | 1-(4-chlorobenzyl)-3-(4-((4- methyl-2-oxopiperazin-1- yl)methyl)phenyl)urea |
| 369 | 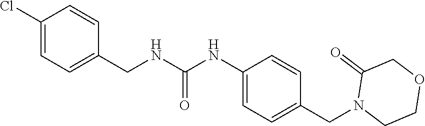 [SMILES: O=C(NCc1ccc(Cl)cc1)Nc1ccc(CN2CCOCC2=O)cc1] | 1-(4-chlorobenzyl)-3-(4-((3- oxomorpholino)methyl)phenyl) urea |
| 370 | 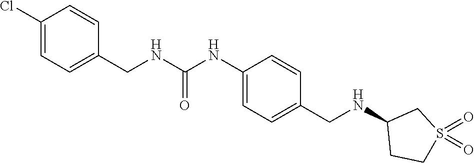 [SMILES: O=C(NCc1ccc(Cl)cc1)Nc1ccc(CN[C@@H]2CCS(=O)(=O)C2)cc1] | (R)-1-(4-chlorobenzyl)-3-(4- (((1,1- dioxidotetrahydrothiophen-3- yl)amino)methyl)phenyl)urea |
| 371 | 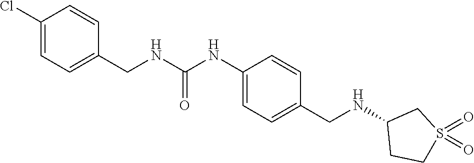 [SMILES: O=C(NCc1ccc(Cl)cc1)Nc1ccc(CN[C@H]2CCS(=O)(=O)C2)cc1] | (S)-1-(4-chlorobenzyl)-3-(4- (((1,1- dioxidotetrahydrothiophen-3- yl)amino)methyl)phenyl)urea |
| 372 | 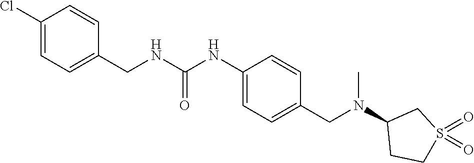 [SMILES: CN(Cc1ccc(NC(=O)NCc2ccc(Cl)cc2)cc1)[C@@H]1CCS(=O)(=O)C1] | (R)-1-(4-chlorobenzyl)-3-(4- (1,1- dioxidotetrahydrothiophen-3- yl)(methyl)amino)methyl)phenyl) urea |
| 373 | 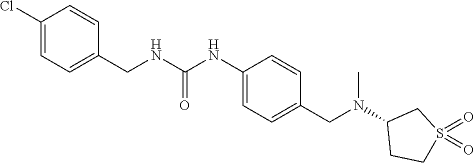 [SMILES: CN(Cc1ccc(NC(=O)NCc2ccc(Cl)cc2)cc1)[C@H]1CCS(=O)(=O)C1] | (S)-1-(4-chlorobenzyl)-3-(4- (1,1- dioxidotetrahydrothiophen-3- yl)(methyl)amino)methyl)phenyl) urea |
| 374 | 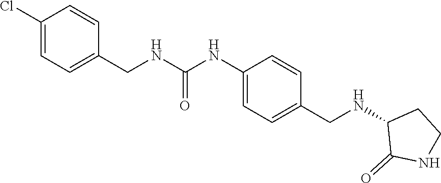 [SMILES: O=C(NCc1ccc(Cl)cc1)Nc1ccc(CN[C@@H]2CCNC2=O)cc1] | (R)-1-(4-chlorobenzyl)-3-(4- (((2-oxopyrrolidin-3- yl)amino)methyl)phenyl)urea |
| 375 | 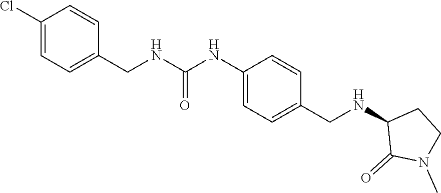 [SMILES: CN1CC[C@H](NCc2ccc(NC(=O)NCc3ccc(Cl)cc3)cc2)C1=O] | (S)-1-(4-chlorobenzyl)-3-(4- (((1-methyl-2-oxopyrrolidin-3- yl)amino)methyl)phenyl)urea |
| 376 | 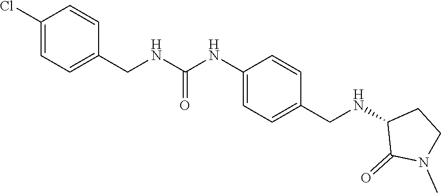 [SMILES: CN1CC[C@@H](NCc2ccc(NC(=O)NCc3ccc(Cl)cc3)cc2)C1=O] | (R)-1-(4-chlorobenzyl)-3-(4- (((1-methyl-2-oxopyrrolidin-3- yl)amino)methyl)phenyl)urea |
| 377 | 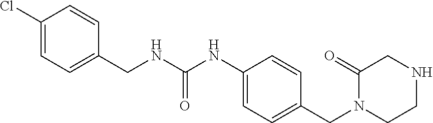 [SMILES: O=C(NCc1ccc(Cl)cc1)Nc1ccc(CN2CCNCC2=O)cc1] | 1-(4-chlorobenzyl)-3-(4-((2- oxopiperazin-1- yl)methyl)phenyl)urea |
| 378 | 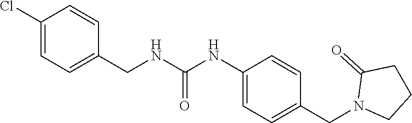 [SMILES: O=C(NCc1ccc(Cl)cc1)Nc1ccc(CN2CCCC2=O)cc1] | 1-(4-chlorobenzyl)-3-(4-((2- oxopyrrolidin-1- yl)methyl)phenyl)urea |
| 379 | 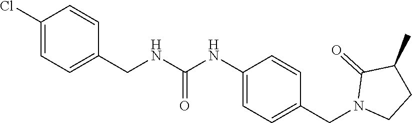 [SMILES: C[C@H]1CCN(Cc2ccc(NC(=O)NCc3ccc(Cl)cc3)cc2)C1=O] | (S)-1-(4-chlorobenzyl)-3-(4-((3- methyl-2-oxopyrrolidin-1- yl)methyl)phenyl)urea |
| 380 | 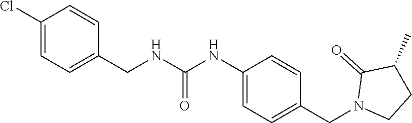 [SMILES: C[C@@H]1CCN(Cc2ccc(NC(=O)NCc3ccc(Cl)cc3)cc2)C1=O] | (R)-1-(4-chlorobenzyl)-3-(4- ((3-methyl-2-oxopyrrolidin-1- yl)methyl)phenyl)urea |
| 381 | 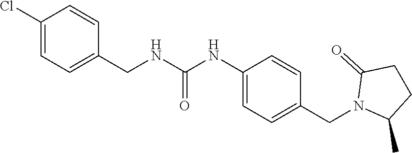 [SMILES: C[C@@H]1CCC(=O)N1Cc1ccc(NC(=O)NCc2ccc(Cl)cc2)cc1] | (R)-1-(4-chlorobenzyl)-3-(4- ((2-methyl-5-oxopyrrolidin-1- yl)methyl)phenyl)urea |
| 382 | 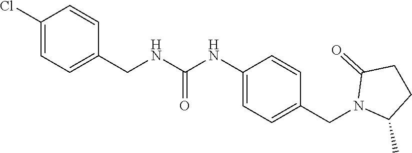 [SMILES: C[C@H]1CCC(=O)N1Cc1ccc(NC(=O)NCc2ccc(Cl)cc2)cc1] | (S)-1-(4-chlorobenzyl)-3-(4-((2- methyl-5-oxopyrrolidin-1- yl)methyl)phenyl)urea |
| 383 | 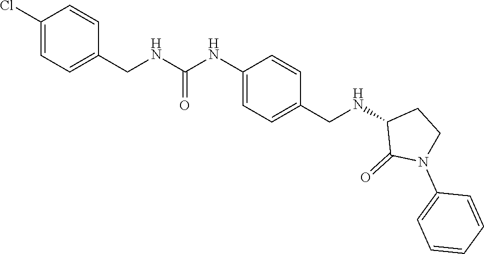 [SMILES: O=C(NCc1ccc(Cl)cc1)Nc1ccc(CN[C@@H]2CCN(c3ccccc3)C2=O)cc1] | (R)-1-(4-chlorobenzyl)-3-(4- (((2-oxo-1-phenylpyrrolidin-3- yl)amino)methyl)phenyl)urea |
| 384 | 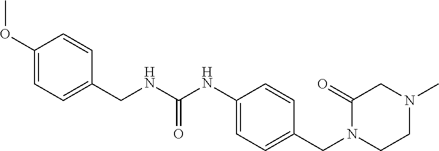 [SMILES: COc1ccc(CNC(=O)Nc2ccc(CN3CCN(C)CC3=O)cc2)cc1] | 1-(4-methoxybenzyl)-3-(4-((4- methyl-2-oxopiperazin-1- yl)methyl)phenyl)urea |
| 385 | 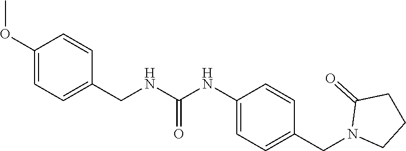 [SMILES: COc1ccc(CNC(=O)Nc2ccc(CN3CCCC3=O)cc2)cc1] | 1-(4-methoxybenzyl)-3-(4-((2- oxopyrrolidin-1- yl)methyl)phenyl)urea |
| 386 | 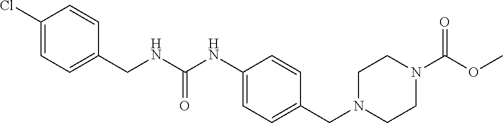 [SMILES: COC(=O)N1CCN(Cc2ccc(NC(=O)NCc3ccc(Cl)cc3)cc2)CC1] | methyl 4-(4-(3-(4- chlorobenzyl)ureido)benzyl) piperazine-1-carboxylate |
| 387 | 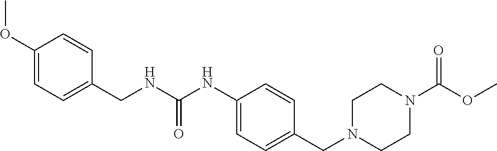 [SMILES: COC(=O)N1CCN(Cc2ccc(NC(=O)NCc3ccc(OC)cc3)cc2)CC1] | methyl 4-(4-(3-(4- methoxybenzyl)ureido)benzyl) piperazine-1-carboxylate |
| 388 | 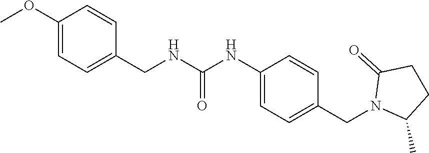 [SMILES: COc1ccc(CNC(=O)Nc2ccc(CN3C(=O)CC[C@@H]3C)cc2)cc1] | (S)-1-(4-methoxybenzyl)-3-(4- ((2-methyl-5-oxopyrrolidin-1- yl)methyl)phenyl)urea |
| 389 | 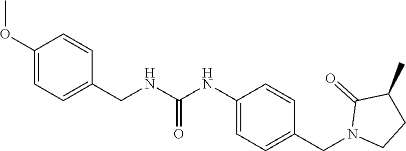 [SMILES: COc1ccc(CNC(=O)Nc2ccc(CN3CC[C@H](C)C3=O)cc2)cc1] | (S)-1-(4-methoxybenzyl)-3-(4- ((3-methyl-2-oxopyrrolidin-1- yl)methyl)phenyl)urea |
| 390 | 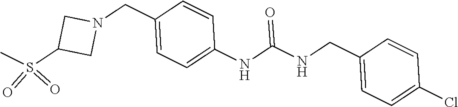 [SMILES: CS(=O)(=O)C1CN(Cc2ccc(NC(=O)NCc3ccc(Cl)cc3)cc2)C1] | 1-(4-chlorobenzyl)-3-(4-((3- (methylsulfonyl)azetidin-1- yl)methyl)phenyl)urea |
| 391 | 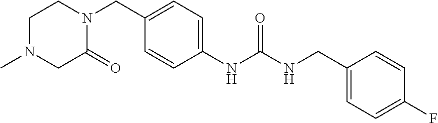 [SMILES: CN1CCN(Cc2ccc(NC(=O)NCc3ccc(F)cc3)cc2)C(=O)C1] | 1-(4-fluorobenzyl)-3-(4-((4- methyl-2-oxopiperazin-1- yl)methyl)phenyl)urea |
| 392 | 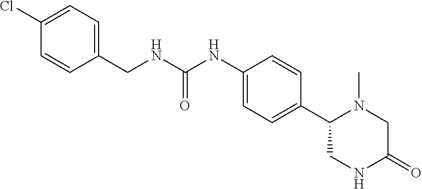 [SMILES: CN1CC(=O)NC[C@@H]1c1ccc(NC(=O)NCc2ccc(Cl)cc2)cc1] | (S)-1-(4-chlorobenzyl)-3-(4-(1- methyl-5-oxopiperazin-2- yl)phenyl)urea |
| 393 | 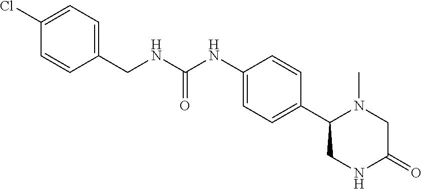 [SMILES: CN1CC(=O)NC[C@H]1c1ccc(NC(=O)NCc2ccc(Cl)cc2)cc1] | (R)-1-(4-chlorobenzyl)-3-(4-(1- methyl-5-oxopiperazin-2- yl)phenyl)urea |
| 394 | 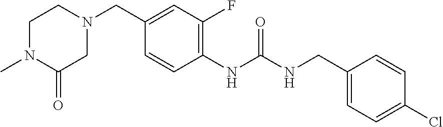 [SMILES: CN1CCN(Cc2ccc(NC(=O)NCc3ccc(Cl)cc3)c(F)c2)CC1=O] | 1-(4-chlorobenzyl)-3-(2-fluoro- 4-((4-methyl-3-oxopiperazin-1- yl)methyl)phenyl)urea |
| 395 | 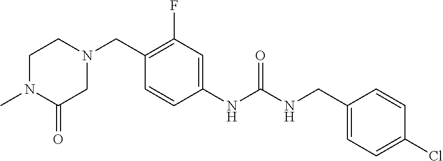 [SMILES: CN1CCN(Cc2ccc(NC(=O)NCc3ccc(Cl)cc3)cc2F)CC1=O] | 1-(4-chlorobenzyl)-3-(3-fluoro- 4-((4-methyl-3-oxopiperazin-1- yl)methyl)phenyl)urea |
| 396 | 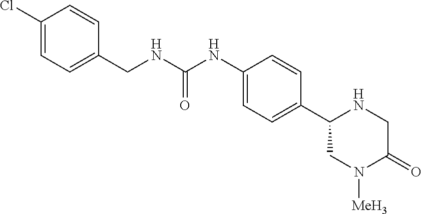 | (S)-1-(4-chlorobenzyl)-3-(4-(4- methyl-5-oxopiperazin-2- yl)phenyl)urea |
| 397 | 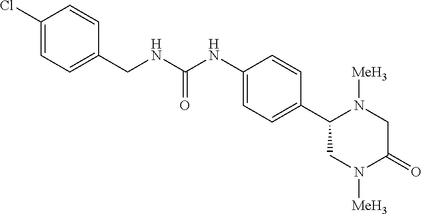 | (S)-1-(4-chlorobenzyl)-3-(4- (1,4-dimethyl-5-oxopiperazin- 2-yl)phenyl)urea |
| 398 | 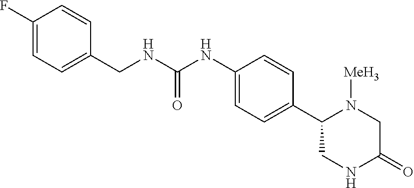 | (S)-1-(4-fluorobenzyl)-3-(4-(1- methyl-5-oxopiperazin-2- yl)phenyl)urea |
| 399 | 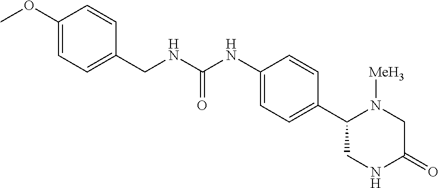 | (S)-1-(4-methoxybenzyl)-3-(4- (1-methyl-5-oxopiperazin-2- yl)phenyl)urea |
| 400 | 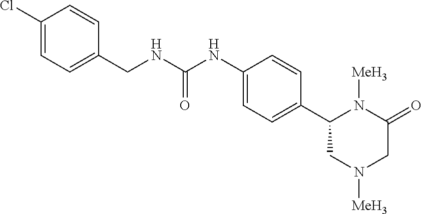 | (S)-1-(4-chlorobenzyl)-3-(4- (1,4-dimethyl-6-oxopiperazin- 2-yl)phenyl)urea |
| 401 | 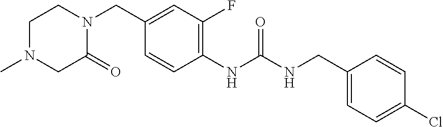 [SMILES: CN1CCN(Cc2ccc(NC(=O)NCc3ccc(Cl)cc3)c(F)c2)C(=O)C1] | 1-(4-chlorobenzyl)-3-(2-fluoro- 4-((4-methyl-2-oxopiperazin-1- yl)methyl)phenyl)urea |
| 402 | 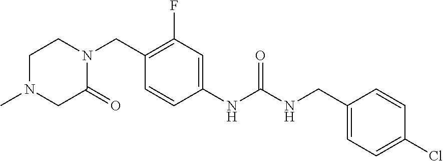 [SMILES: CN1CCN(Cc2ccc(NC(=O)NCc3ccc(Cl)cc3)cc2F)C(=O)C1] | 1-(4-chlorobenzyl)-3-(3-fluoro- 4-((4-methyl-2-oxopiperazin-1- yl)methyl)phenyl)urea |
| 403 | 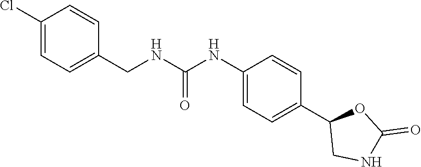 [SMILES: O=C(NCc1ccc(Cl)cc1)Nc1ccc([C@@H]2CNC(=O)O2)cc1] | (R)-1-(4-chlorobenzyl)-3-(4-(2- oxooxazolidin-5-yl)phenyl)urea |
| 404 | 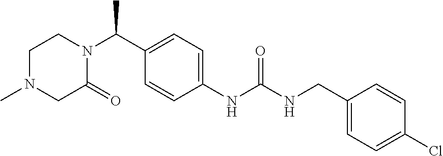 [SMILES: C[C@@H](c1ccc(NC(=O)NCc2ccc(Cl)cc2)cc1)N1CCN(C)CC1=O] | (S)-1-(4-chlorobenzyl)-3-(4-(1- (4-methyl-2-oxopiperazin-1- yl)ethyl)phenyl)urea |
| 405 | 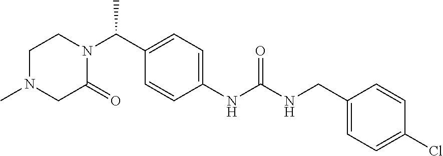 [SMILES: C[C@H](c1ccc(NC(=O)NCc2ccc(Cl)cc2)cc1)N1CCN(C)CC1=O] | (R)-1-(4-chlorobenzyl)-3-(4-(1- (4-methyl-2-oxopiperazin-1- yl)ethyl)phenyl)urea |
| 406 | 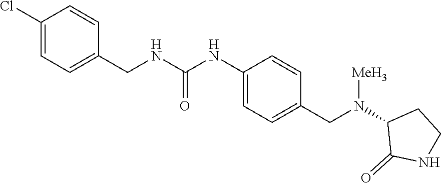 | (R)-1-(4-chlorobenzyl)-3-(4- ((methyl(2-oxopyrrolidin-3- yl)amino)methyl)phenyl)urea |
| 407 | 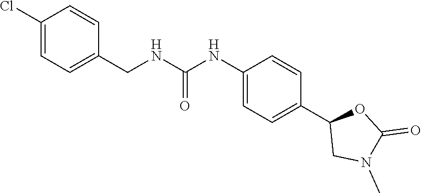 [SMILES: CN1C[C@@H](c2ccc(NC(=O)NCc3ccc(Cl)cc3)cc2)OC1=O] | (R)-1-(4-chlorobenzyl)-3-(4-(3- methyl-2-oxooxazolidin-5- yl)phenyl)urea |
| 408 | 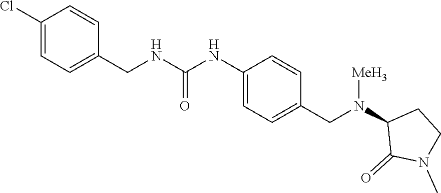 | (S)-1-(4-chlorobenzyl)-3-(4- ((methyl(1-methyl-2- oxopyrrolidin-3- yl)amino)methyl)phenyl)urea |
| 409 | 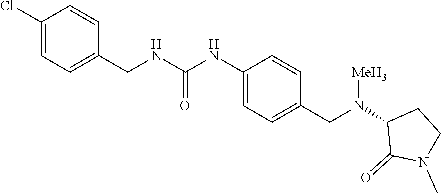 | (R)-1-(4-chlorobenzyl)-3-(4- ((methyl(1-methyl-2- oxopyrrolidin-3- yl)amino)methyl)phenyl)urea |
| 410 | 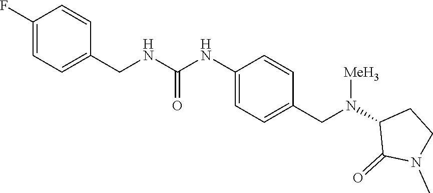 | (R)-1-(4-fluorobenzyl)-3-(4- ((methyl(1-methyl-2- oxopyrrolidin-3- yl)amino)methyl)phenyl)urea |
| 411 | 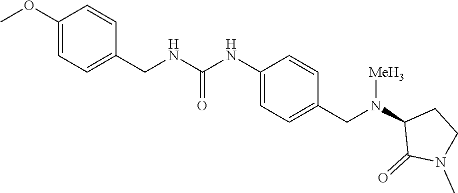 | (S)-1-(4-methoxybenzyl)-3-(4- ((methyl(1-methyl-2- oxopyrrolidin-3- yl)amino)methyl)phenyl)urea |
| 412 | 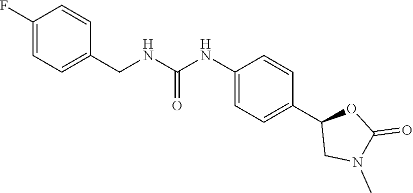 [SMILES: CN1C[C@@H](c2ccc(NC(=O)NCc3ccc(F)cc3)cc2)OC1=O] | (R)-1-(4-fluorobenzyl)-3-(4-(3- methyl-2-oxooxazolidin-5- yl)phenyl)urea |
| 413 | 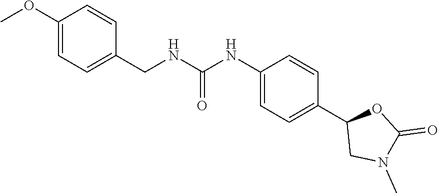 [SMILES: COc1ccc(CNC(=O)Nc2ccc([C@@H]3CN(C)C(=O)O3)cc2)cc1] | (R)-1-(4-methoxybenzyl)-3-(4- (3-methyl-2-oxooxazolidin-5- yl)phenyl)urea |
| 414 | 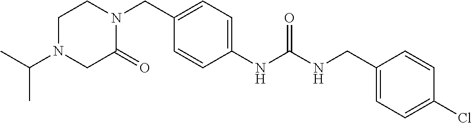 [SMILES: CC(C)N1CCN(Cc2ccc(NC(=O)NCc3ccc(Cl)cc3)cc2)C(=O)C1] | 1-(4-chlorobenzyl)-3-(4-((4- isopropyl-2-oxopiperazin-1- yl)methyl)phenyl)urea |
| 415 | 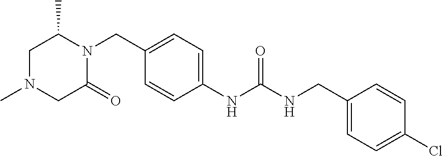 [SMILES: C[C@H]1CN(C)CC(=O)N1Cc1ccc(NC(=O)NCc2ccc(Cl)cc2)cc1] | (S)-1-(4-chlorobenzyl)-3-(4- ((2,4-dimethyl-6-oxopiperazin- 1-yl)methyl)phenyl)urea |
| 416 | 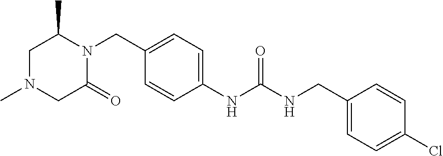 [SMILES: C[C@@H]1CN(C)CC(=O)N1Cc1ccc(NC(=O)NCc2ccc(Cl)cc2)cc1] | (R)-1-(4-chlorobenzyl)-3-(4- ((2,4-dimethyl-6-oxopiperazin- 1-yl)methyl)phenyl)urea |
| 417 | 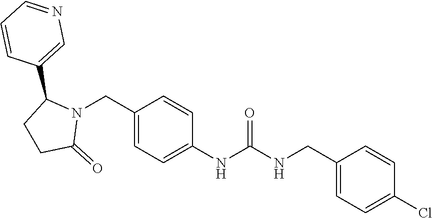 [SMILES: O=C(NCc1ccc(Cl)cc1)Nc1ccc(CN2C(=O)CC[C@H]2c2cccnc2)cc1] | (S)-1-(4-chlorobenzyl)-3-(4-((2- oxo-5-(pyridin-3-yl)pyrrolidin- 1-yl)methyl)phenyl)urea |
| 418 | 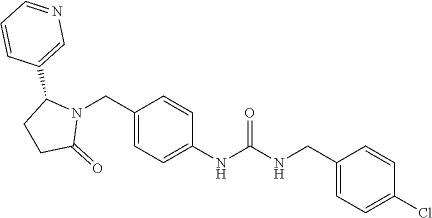 [SMILES: O=C(NCc1ccc(Cl)cc1)Nc1ccc(CN2C(=O)CC[C@@H]2c2cccnc2)cc1] | (R)-1-(4-chlorobenzyl)-3-(4- ((2-oxo-5-(pyridin-3- yl)pyrrolidin-1- yl)methyl)phenyl)urea |
| 419 | 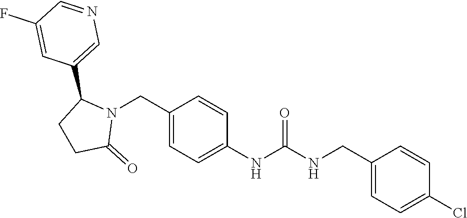 [SMILES: O=C(NCc1ccc(Cl)cc1)Nc1ccc(CN2C(=O)CC[C@H]2c2cncc(F)c2)cc1] | (S)-1-(4-chlorobenzyl)-3-(4-((2- (5-fluoropyridin-3-yl)-5- oxopyrrolidin-1- yl)methyl)phenyl)urea |
| 420 | 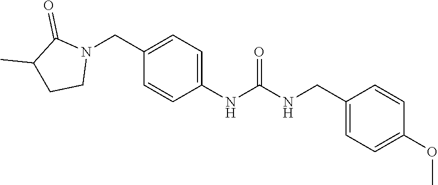 | 1-(4-methoxybenzyl)-3-(4-((3- methyl-2-oxopyrrolidin-1- yl)methyl)phenyl)urea |
| 421 | 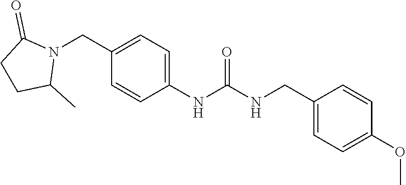 [SMILES: COc1ccc(CNC(=O)Nc2ccc(CN3C(=O)CCC3C)cc2)cc1] | 1-(4-methoxybenzyl)-3-(4-((2- methyl-5-oxopyrrolidin-1- yl)methyl)phenyl)urea |
| 422 | 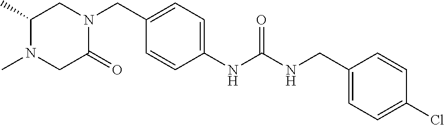 [SMILES: C[C@@H]1CN(Cc2ccc(NC(=O)NCc3ccc(Cl)cc3)cc2)C(=O)CN1C] | (R)-1-(4-chlorobenzyl)-3-(4- ((4,5-dimethyl-2-oxopiperazin- 1-yl)methyl)phenyl)urea |
| 423 | 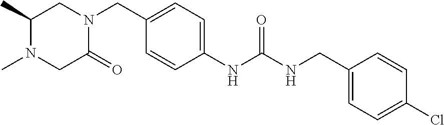 [SMILES: C[C@H]1CN(Cc2ccc(NC(=O)NCc3ccc(Cl)cc3)cc2)C(=O)CN1C] | (S)-1-(4-chlorobenzyl)-3-(4- ((4,5-dimethyl-2-oxopiperazin- 1-yl)methyl)phenyl)urea |
| 424 | 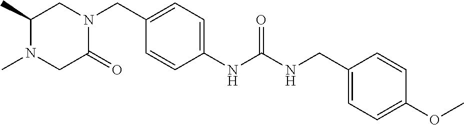 | (S)-1-(4-((4,5-dimethyl-2- oxopiperazin-1- yl)methyl)phenyl)-3-(4- methoxybenzyl)urea |
| 425 | 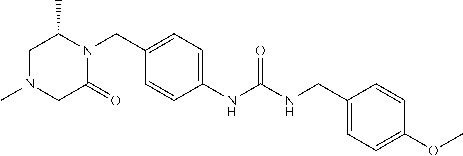 | (S)-1-(4-((2,4-dimethyl-6- oxopiperazin-1- yl)methyl)phenyl)-3-(4- methoxybenzyl)urea |
| 426 | 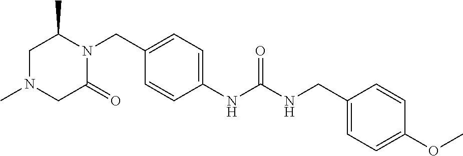 | (R)-1-(4-((2,4-dimethyl-6- oxopiperazin-1- yl)methyl)phenyl)-3-(4- methoxybenzyl)urea |
| 427 | 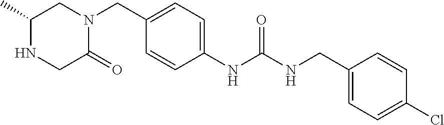 [SMILES: C[C@@H]1CN(Cc2ccc(NC(=O)NCc3ccc(Cl)cc3)cc2)C(=O)CN1] | (R)-1-(4-chlorobenzyl)-3-(4- ((5-methyl-2-oxopiperazin-1- yl)methyl)phenyl)urea |
| 428 | 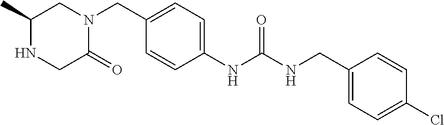 [SMILES: C[C@H]1CN(Cc2ccc(NC(=O)NCc3ccc(Cl)cc3)cc2)C(=O)CN1] | (S)-1-(4-chlorobenzyl)-3-(4-((5- methyl-2-oxopiperazin-1- yl)methyl)phenyl)urea |
| 429 | 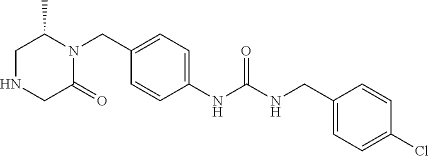 [SMILES: C[C@H]1CNCC(=O)N1Cc1ccc(NC(=O)NCc2ccc(Cl)cc2)cc1] | (S)-1-(4-chlorobenzyl)-3-(4-((2- methyl-6-oxopiperazin-1- yl)methyl)phenyl)urea |
| 430 | 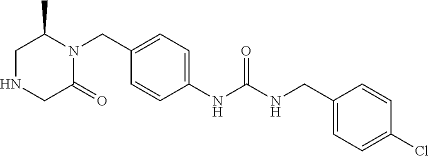 [SMILES: C[C@@H]1CNCC(=O)N1Cc1ccc(NC(=O)NCc2ccc(Cl)cc2)cc1] | (R)-1-(4-chlorobenzyl)-3-(4- ((2-methyl-6-oxopiperazin-1- yl)methyl)phenyl)urea |
| 431 | 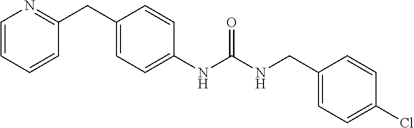 [SMILES: O=C(NCc1ccc(Cl)cc1)Nc1ccc(Cc2ccccn2)cc1] | 1-(4-chlorobenzyl)-3-(4- (pyridin-2- ylmethyl)phenyl)urea |
| 432 | 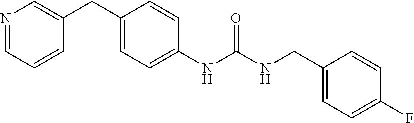 [SMILES: O=C(NCc1ccc(F)cc1)Nc1ccc(Cc2cccnc2)cc1] | 1-(4-fluorobenzyl)-3-(4- (pyridin-3- ylmethyl)phenyl)urea |
| 433 | 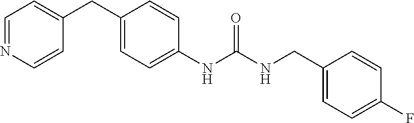 [SMILES: O=C(NCc1ccc(F)cc1)Nc1ccc(Cc2ccncc2)cc1] | 1-(4-fluorobenzyl)-3-(4- (pyridin-4- ylmethyl)phenyl)urea |
| 434 | 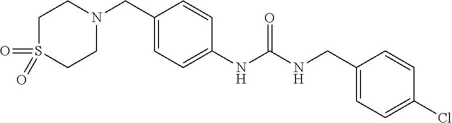 [SMILES: O=C(NCc1ccc(Cl)cc1)Nc1ccc(CN2CCS(=O)(=O)CC2)cc1] | 1-(4-chlorobenzyl)-3-(4-((1,1- dioxidothiomorpholino)methyl) phenyl)urea |
| 435 | 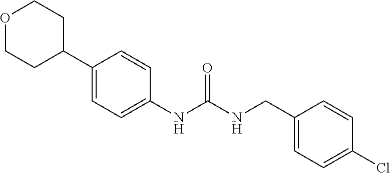 [SMILES: O=C(NCc1ccc(Cl)cc1)Nc1ccc(C2CCOCC2)cc1] | 1-(4-chlorobenzyl)-3-(4- (tetrahydro-2H-pyran-4- yl)phenyl)urea |
| 436 | 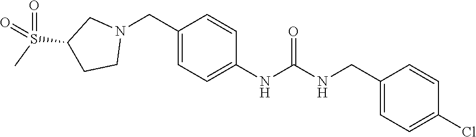 [SMILES: CS(=O)(=O)[C@H]1CCN(Cc2ccc(NC(=O)NCc3ccc(Cl)cc3)cc2)C1] | (S)-1-(4-chlorobenzyl)-3-(4-((3- (methylsulfonyl)pyrrolidin-1- yl)methyl)phenyl)urea |
| 437 | 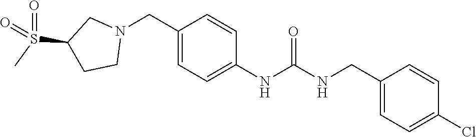 [SMILES: CS(=O)(=O)[C@@H]1CCN(Cc2ccc(NC(=O)NCc3ccc(Cl)cc3)cc2)C1] | (R)-1-(4-chlorobenzyl)-3-(4- ((3-(methylsulfonyl)pyrrolidin- 1-yl)methyl)phenyl)urea |
| 438 | 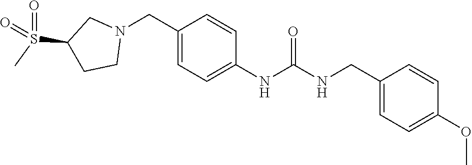 [SMILES: COc1ccc(CNC(=O)Nc2ccc(CN3CC[C@@H](S(C)(=O)=O)C3)cc2)cc1] | (R)-1-(4-methoxybenzyl)-3-(4- ((3-(methylsulfonyl)pyrrolidin- 1-yl)methyl)phenyl)urea |
| 439 | 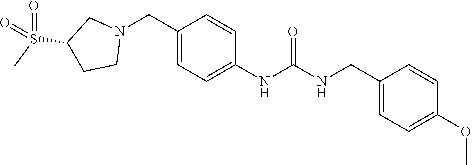 [SMILES: COc1ccc(CNC(=O)Nc2ccc(CN3CC[C@H](S(C)(=O)=O)C3)cc2)cc1] | (S)-1-(4-methoxybenzyl)-3-(4- ((3-(methylsulfonyl)pyrrolidin- 1-yl)methyl)phenyl)urea |
| 440 | 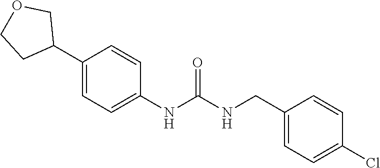 [SMILES: O=C(NCc1ccc(Cl)cc1)Nc1ccc(C2CCOC2)cc1] | 1-(4-chlorobenzyl)-3-(4- (tetrahydrofuran-3- yl)phenyl)urea |
| 441 | 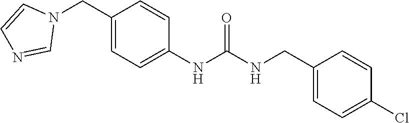 [SMILES: O=C(NCc1ccc(Cl)cc1)Nc1ccc(Cn2ccnc2)cc1] | 1-(4-((1H-imidazol-1- yl)methyl)phenyl)-3-(4- chlorobenzyl)urea |
| 442 | 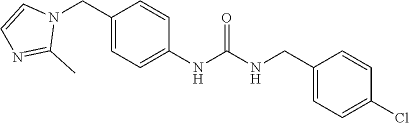 [SMILES: Cc1nccn1Cc1ccc(NC(=O)NCc2ccc(Cl)cc2)cc1] | 1-(4-chlorobenzyl)-3-(4-((2- methyl-1H-imidazol-1- yl)methyl)phenyl)urea |
| 443 | 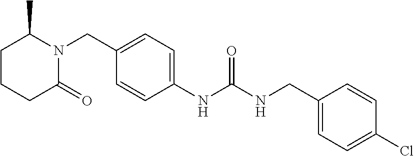 [SMILES: C[C@@H]1CCCC(=O)N1Cc1ccc(NC(=O)NCc2ccc(Cl)cc2)cc1] | (R)-1-(4-chlorobenzyl)-3-(4- ((2-methyl-6-oxopiperidin-1- yl)methyl)phenyl)urea |
| 444 | 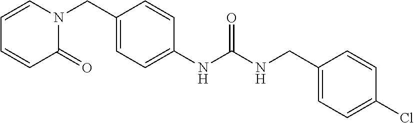 [SMILES: O=C(NCc1ccc(Cl)cc1)Nc1ccc(Cn2ccccc2=O)cc1] | 1-(4-chlorobenzyl)-3-(4-((2- oxopyridin-1(2H)- yl)methyl)phenyl)urea |
| 445 | 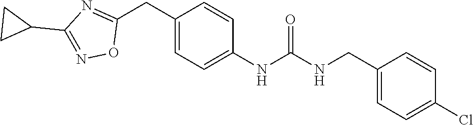 [SMILES: O=C(NCc1ccc(Cl)cc1)Nc1ccc(Cc2nc(C3CC3)no2)cc1] | 1-(4-chlorobenzyl)-3-(4-((3- cyclopropyl-1,2,4-oxadiazol-5- yl)methyl)phenyl)urea |
| 446 | 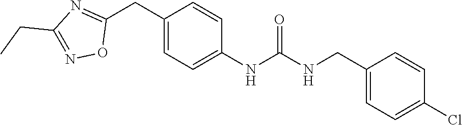 [SMILES: CCc1noc(Cc2ccc(NC(=O)NCc3ccc(Cl)cc3)cc2)n1] | 1-(4-chlorobenzyl)-3-(4-((3- ethyl-1,2,4-oxadiazol-5- yl)methyl)phenyl)urea |
| 447 | 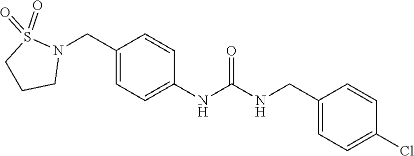 [SMILES: O=C(NCc1ccc(Cl)cc1)Nc1ccc(CN2CCCS2(=O)=O)cc1] | 1-(4-chlorobenzyl)-3-(4-((1,1- dioxidoisothiazolidin-2- yl)methyl)phenyl)urea |
| 448 | 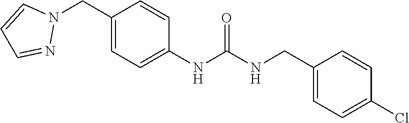 [SMILES: O=C(NCc1ccc(Cl)cc1)Nc1ccc(Cn2cccn2)cc1] | 1-(4-((1H-pyrazol-1- yl)methyl)phenyl)-3-(4- chlorobenzyl)urea |
| 449 | 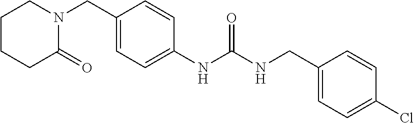 [SMILES: O=C(NCc1ccc(Cl)cc1)Nc1ccc(CN2CCCCC2=O)cc1] | 1-(4-chlorobenzyl)-3-(4-((2- oxopiperidin-1- yl)methyl)phenyl)urea |
| 450 | 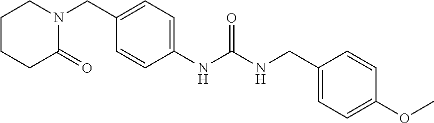 [SMILES: COc1ccc(CNC(=O)Nc2ccc(CN3CCCCC3=O)cc2)cc1] | 1-(4-methoxybenzyl)-3-(4-((2- oxopiperidin-1- yl)methyl)phenyl)urea |
| 451 | 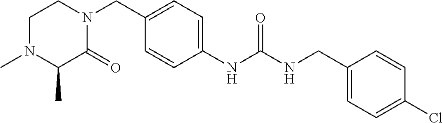 [SMILES: C[C@@H]1C(=O)N(Cc2ccc(NC(=O)NCc3ccc(Cl)cc3)cc2)CCN1C] | (R)-1-(4-chlorobenzyl)-3-(4- ((3,4-dimethyl-2-oxopiperazin- 1-yl)methyl)phenyl)urea |
| 452 | 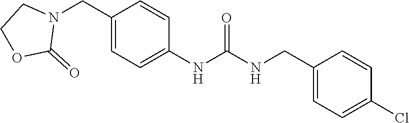 [SMILES: O=C(NCc1ccc(Cl)cc1)Nc1ccc(CN2CCOC2=O)cc1] | 1-(4-chlorobenzyl)-3-(4-((2- oxooxazolidin-3- yl)methyl)phenyl)urea |
| 453 | 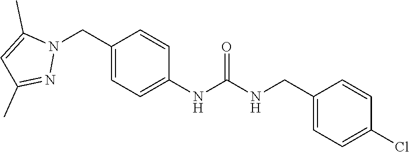 [SMILES: Cc1cc(C)n(Cc2ccc(NC(=O)NCc3ccc(Cl)cc3)cc2)n1] | 1-(4-chlorobenzyl)-3-(4-((3,5- dimethyl-1H-pyrazol-1- yl)methyl)phenyl)urea |
| 454 | 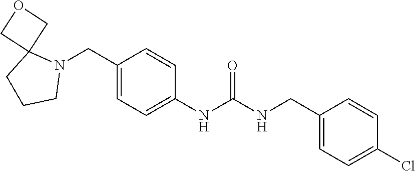 [SMILES: O=C(NCc1ccc(Cl)cc1)Nc1ccc(CN2CCCC23COC3)cc1] | 1-(4-((2-oxa-5- azaspiro[3.4]octan-5- yl)methyl)phenyl)-3-(4- chlorobenzyl)urea |
| 455 | 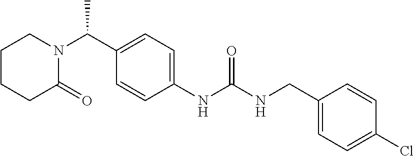 [SMILES: C[C@H](c1ccc(NC(=O)NCc2ccc(Cl)cc2)cc1)N1CCCCC1=O] | (R)-1-(4-chlorobenzyl)-3-(4-(1- (2-oxopiperidin-1- yl)ethyl)phenyl)urea |
| 456 | 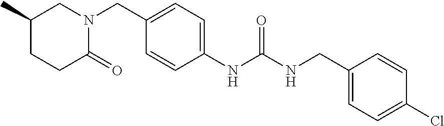 [SMILES: C[C@@H]1CCC(=O)N(Cc2ccc(NC(=O)NCc3ccc(Cl)cc3)cc2)C1] | (R)-1-(4-chlorobenzyl)-3-(4- ((5-methyl-2-oxopiperidin-1- yl)methyl)phenyl)urea |
| 457 | 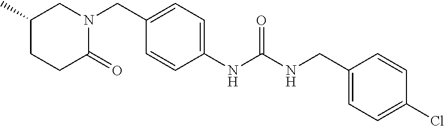 [SMILES: C[C@H]1CCC(=O)N(Cc2ccc(NC(=O)NCc3ccc(Cl)cc3)cc2)C1] | (S)-1-(4-chlorobenzyl)-3-(4-((5- methyl-2-oxopiperidin-1- yl)methyl)phenyl)urea |
| 458 | 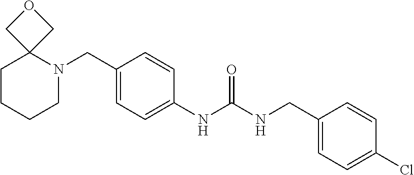 [SMILES: O=C(NCc1ccc(Cl)cc1)Nc1ccc(CN2CCCCC23COC3)cc1] | 1-(4-((2-oxa-5- azaspiro[3.5]nonan-5- yl)methyl)phenyl)-3-(4- chlorobenzyl)urea |
| 459 | 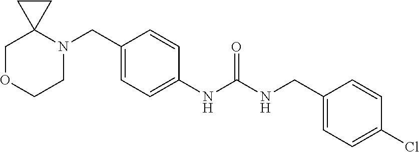 [SMILES: O=C(NCc1ccc(Cl)cc1)Nc1ccc(CN2CCOCC23CC3)cc1] | 1-(4-((7-oxa-4- azaspiro[2.5]octan-4- yl)methyl)phenyl)-3-(4- chlorobenzyl)urea |
| 460 | 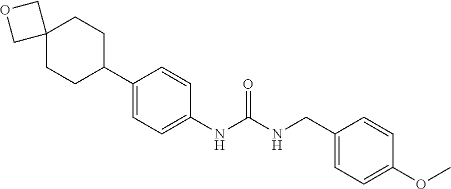 [SMILES: COc1ccc(CNC(=O)Nc2ccc(C3CCC4(CC3)COC4)cc2)cc1] | 1-(4-(2-oxaspiro[3.5]nonan-7- yl)phenyl)-3-(4- methoxybenzyl)urea |
| 461 | 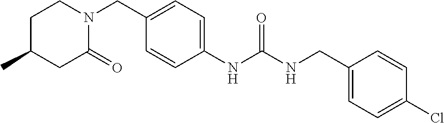 [SMILES: C[C@H]1CCN(Cc2ccc(NC(=O)NCc3ccc(Cl)cc3)cc2)C(=O)C1] | (S)-1-(4-chlorobenzyl)-3-(4-((4- methyl-2-oxopiperidin-1- yl)methyl)phenyl)urea |
| 462 | 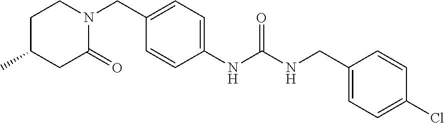 [SMILES: C[C@@H]1CCN(Cc2ccc(NC(=O)NCc3ccc(Cl)cc3)cc2)C(=O)C1] | (R)-1-(4-chlorobenzyl)-3-(4- ((4-methyl-2-oxopiperidin-1- yl)methyl)phenyl)urea |
| 463 | 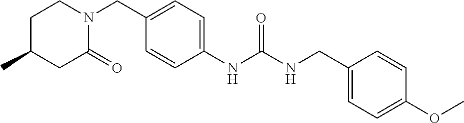 | (S)-1-(4-methoxybenzyl)-3-(4- ((4-methyl-2-oxopiperidin-1- yl)methyl)phenyl)urea |
| 464 | 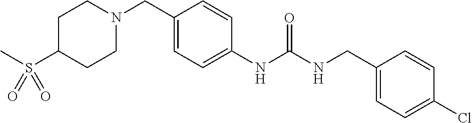 [SMILES: CS(=O)(=O)C1CCN(Cc2ccc(NC(=O)NCc3ccc(Cl)cc3)cc2)CC1] | 1-(4-chlorobenzyl)-3-(4-((4- (methylsulfonyl)piperidin-1- yl)methyl)phenyl)urea |
| 465 | 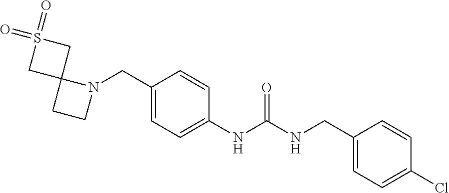 [SMILES: O=C(NCc1ccc(Cl)cc1)Nc1ccc(CN2CCC23CS(=O)(=O)C3)cc1] | 1-(4-chlorobenzyl)-3-(4-((6,6- dioxido-6-thia-1- azaspiro[3.3]heptan-1- yl)methyl)phenyl)urea |
| 466 | 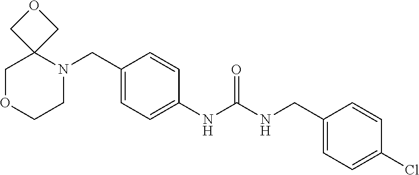 [SMILES: O=C(NCc1ccc(Cl)cc1)Nc1ccc(CN2CCOCC23COC3)cc1] | 1-(4-((2,8-dioxa-5- azaspiro[3.5]nonan-5- yl)methyl)phenyl)-3-(4- chlorobenzyl)urea |
| 467 | 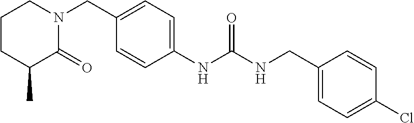 [SMILES: C[C@H]1CCCN(Cc2ccc(NC(=O)NCc3ccc(Cl)cc3)cc2)C1=O] | (S)-1-(4-chlorobenzyl)-3-(4-((3- methyl-2-oxopiperidin-1- yl)methyl)phenyl)urea |
| 468 | 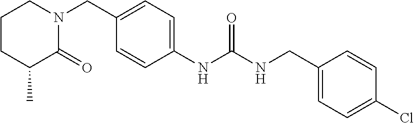 [SMILES: C[C@@H]1CCCN(Cc2ccc(NC(=O)NCc3ccc(Cl)cc3)cc2)C1=O] | (R)-1-(4-chlorobenzyl)-3-(4- ((3-methyl-2-oxopiperidin-1- yl)methyl)phenyl)urea |
| 469 | 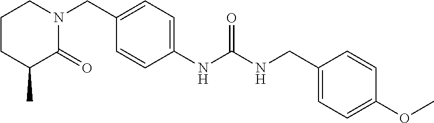 [SMILES: COc1ccc(CNC(=O)Nc2ccc(CN3CCC[C@H](C)C3=O)cc2)cc1] | (S)-1-(4-methoxybenzyl)-3-(4- ((3-methyl-2-oxopiperidin-1- yl)methyl)phenyl)urea |
| 470 | 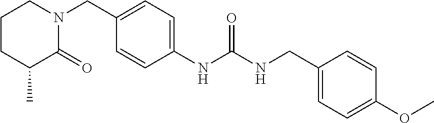 [SMILES: COc1ccc(CNC(=O)Nc2ccc(CN3CCC[C@@H](C)C3=O)cc2)cc1] | (R)-1-(4-methoxybenzyl)-3-(4- ((3-methyl-2-oxopiperidin-1- yl)methyl)phenyl)urea |
| 471 | 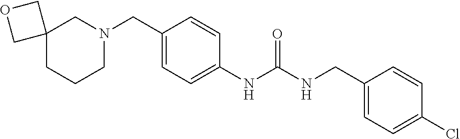 [SMILES: O=C(NCc1ccc(Cl)cc1)Nc1ccc(CN2CCCC3(COC3)C2)cc1] | 1-(4-((2-oxa-6- azaspiro[3.5]nonan-6- yl)methyl)phenyl)-3-(4- chlorobenzyl)urea |
| 472 | 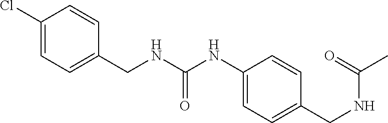 [SMILES: CC(=O)NCc1ccc(NC(=O)NCc2ccc(Cl)cc2)cc1] | N-(4-(3-(4- chlorobenzyl)ureido)benzyl) acetamide |
| 473 | 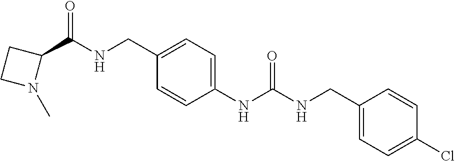 [SMILES: CN1CC[C@H]1C(=O)NCc1ccc(NC(=O)NCc2ccc(Cl)cc2)cc1] | (S)-N-(4-(3-(4- chlorobenzyl)ureido)benzyl)-1- methylazetidine-2-carboxamide |
| 474 | 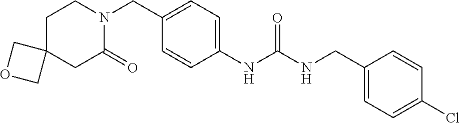 [SMILES: O=C(NCc1ccc(Cl)cc1)Nc1ccc(CN2CCC3(COC3)CC2=O)cc1] | 1-(4-chlorobenzyl)-3-(4-((6- oxo-2-oxa-7- azaspiro[3.5]nonan-7- yl)methyl)phenyl)urea |
| 475 | 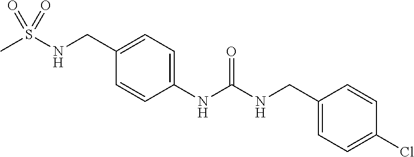 [SMILES: CS(=O)(=O)NCc1ccc(NC(=O)NCc2ccc(Cl)cc2)cc1] | N-(4-(3-(4- chlorobenzyl)ureido)benzyl) methanesulfonamide |
| 476 | 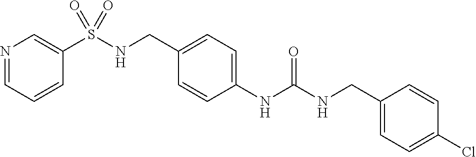 [SMILES: O=C(NCc1ccc(Cl)cc1)Nc1ccc(CNS(=O)(=O)c2cccnc2)cc1] | N-(4-(3-(4- chlorobenzyl)ureido)benzyl) pyridine-3-sulfonamide |
| 477 | 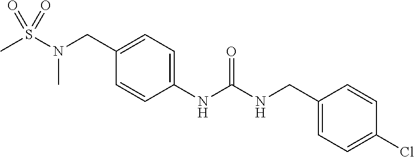 [SMILES: CN(Cc1ccc(NC(=O)NCc2ccc(Cl)cc2)cc1)S(C)(=O)=O] | N-(4-(3-(4- chlorobenzyl)ureido)benzyl)-N- methylmethanesulfonamide |
| 478 | 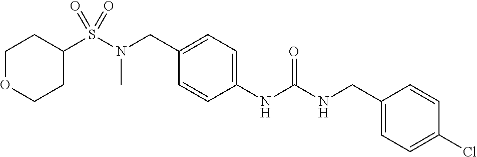 [SMILES: CN(Cc1ccc(NC(=O)NCc2ccc(Cl)cc2)cc1)S(=O)(=O)C1CCOCC1] | N-(4-(3-(4- chlorobenzyl)ureido)benzyl)-N- methyltetrahydro-2H-pyran-4- sulfonamide |
| 479 | 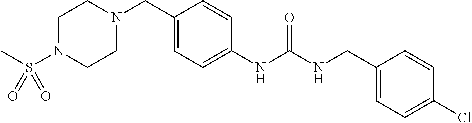 [SMILES: CS(=O)(=O)N1CCN(Cc2ccc(NC(=O)NCc3ccc(Cl)cc3)cc2)CC1] | 1-(4-chlorobenzyl)-3-(4-((4- (methylsulfonyl)piperazin-1- yl)methyl)phenyl)urea |
| 480 | 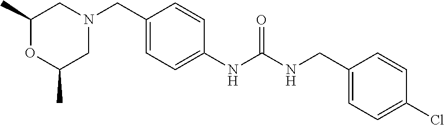 [SMILES: C[C@@H]1CN(Cc2ccc(NC(=O)NCc3ccc(Cl)cc3)cc2)C[C@H](C)O1] | 1-(4-chlorobenzyl)-3-(4- (((2R,6S)-2,6- dimethylmorpholino)methyl) phenyl)urea |
| 481 | 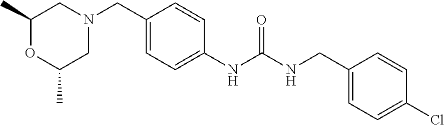 [SMILES: C[C@H]1CN(Cc2ccc(NC(=O)NCc3ccc(Cl)cc3)cc2)C[C@H](C)O1] | 1-(4-chlorobenzyl)-3-(4- (((2S,6S)-2,6- dimethylmorpholino)methyl) phenyl)urea |
| 482 | 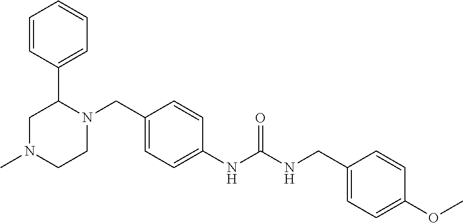 | 1-(4-methoxybenzyl)-3-(4-((4- methyl-2-phenylpiperazin-1- yl)methyl)phenyl)urea |
| 483 | 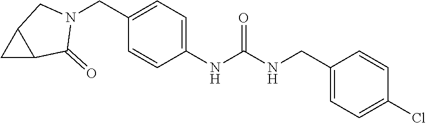 [SMILES: O=C(NCc1ccc(Cl)cc1)Nc1ccc(CN2CC3CC3C2=O)cc1] | 1-(4-chlorobenzyl)-3-(4-((2- oxo-3-azabicyclo[3.1.0]hexan- 3-yl)methyl)phenyl)urea |
| 484 | 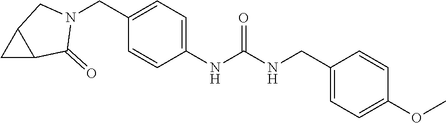 [SMILES: COc1ccc(CNC(=O)Nc2ccc(CN3CC4CC4C3=O)cc2)cc1] | 1-(4-methoxybenzyl)-3-(4-((2- oxo-3-azabicyclo[3.1.0]hexan- 3-yl)methyl)phenyl)urea |
| 485 | 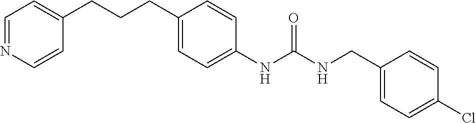 [SMILES: O=C(NCc1ccc(Cl)cc1)Nc1ccc(CCCc2ccncc2)cc1] | 1-(4-chlorobenzyl)-3-(4-(2- (pyridin-4- yloxy)ethyl)phenyl)urea |
| 486 | 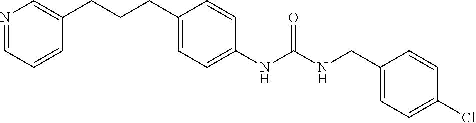 [SMILES: O=C(NCc1ccc(Cl)cc1)Nc1ccc(CCCc2cccnc2)cc1] | 1-(4-chlorobenzyl)-3-(4-(2- (pyridin-3- yloxy)ethyl)phenyl)urea |
| 487 | 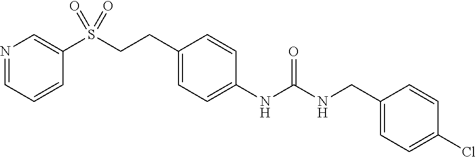 [SMILES: O=C(NCc1ccc(Cl)cc1)Nc1ccc(CCS(=O)(=O)c2cccnc2)cc1] | 1-(4-chlorobenzyl)-3-(4-(2- (pyridin-3- ylsulfonyl)ethyl)phenyl)urea |
| 488 | 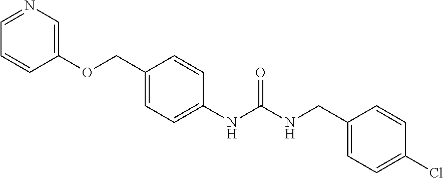 [SMILES: O=C(NCc1ccc(Cl)cc1)Nc1ccc(COc2cccnc2)cc1] | 1-(4-chlorobenzyl)-3-(4- ((pyridin-3- yloxy)methyl)phenyl)urea |
| 489 | 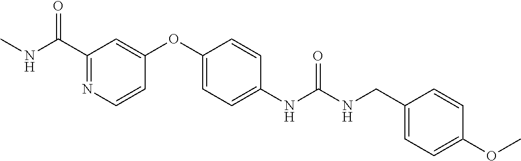 | 4-(4-(3-(4- methoxybenzyl)ureido)phenoxy)- N-methylpicolinamide |
| 490 | 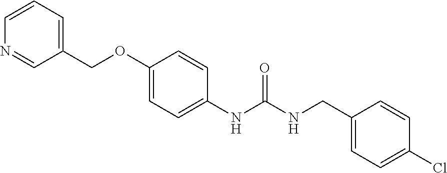 [SMILES: O=C(NCc1ccc(Cl)cc1)Nc1ccc(OCc2cccnc2)cc1] | 1-(4-chlorobenzyl)-3-(4- (pyridin-3- ylmethoxy)phenyl)urea |
| 491 | 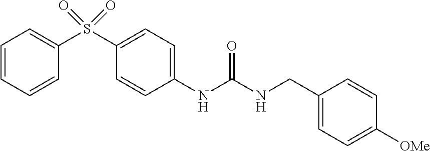 [SMILES: COc1ccc(CNC(=O)Nc2ccc(S(=O)(=O)c3ccccc3)cc2)cc1] | 1-(4-methoxybenzyl)-3-(4- (phenylsulfonyl)phenyl)urea |
| 492 | 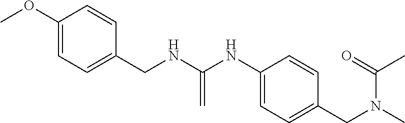 [SMILES: C=C(NCc1ccc(OC)cc1)Nc1ccc(CN(C)C(C)=O)cc1] | N-(4-(3-(4- methoxybenzyl)ureido)benzyl)- N-methylacetamide |
| 493 | 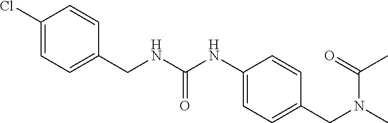 [SMILES: CC(=O)N(C)Cc1ccc(NC(=O)NCc2ccc(Cl)cc2)cc1] | N-(4-(3-(4- chlorobenzyl)ureido)benzyl)-N- methylacetamide |
| 494 | 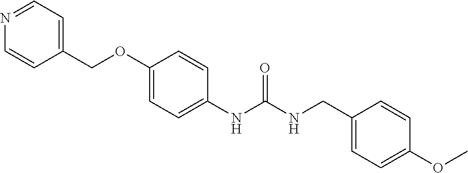 [SMILES: COc1ccc(CNC(=O)Nc2ccc(OCc3ccncc3)cc2)cc1] | 1-(4-methoxybenzyl)-3-(4- (pyridin-4- ylmethoxy)phenyl)urea |
| 495 | 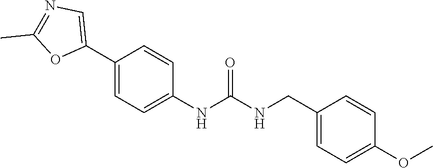 | 1-(4-methoxybenzyl)-3-(4-(2- methyloxazol-5-yl)phenyl)urea |
| 496 | 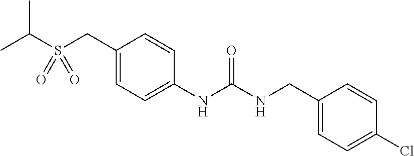 [SMILES: CC(C)S(=O)(=O)Cc1ccc(NC(=O)NCc2ccc(Cl)cc2)cc1] | 1-(4-chlorobenzyl)-3-(4- ((isopropylsulfonyl)methyl) phenyl)urea |
| 497 | 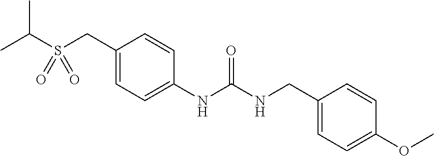 [SMILES: COc1ccc(CNC(=O)Nc2ccc(CS(=O)(=O)C(C)C)cc2)cc1] | 1-(4-((isopropylsulfonyl)methyl) phenyl)-3-(4-methoxybenzyl)urea |
| 498 | 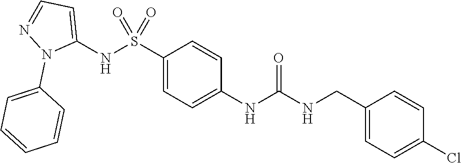 [SMILES: O=C(NCc1ccc(Cl)cc1)Nc1ccc(S(=O)(=O)Nc2ccnn2-c2ccccc2)cc1] | 4-(3-(4-chlorobenzyl)ureido)- N-(1-phenyl-1H-pyrazol-5- yl)benzenesulfonamide |
| 499 | 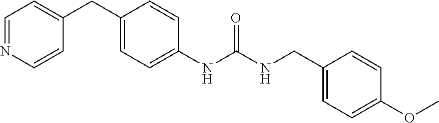 [SMILES: COc1ccc(CNC(=O)Nc2ccc(Cc3ccncc3)cc2)cc1] | 1-(4-methoxybenzyl)-3-(4- (pyridin-4- ylmethyl)phenyl)urea |
| 500 | 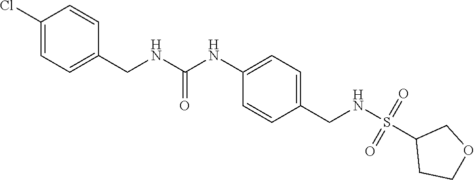 [SMILES: O=C(NCc1ccc(Cl)cc1)Nc1ccc(CNS(=O)(=O)C2CCOC2)cc1] | N-(4-(3-(4- chlorobenzyl)ureido)benzyl) tetrahydrofuran-3-sulfonamide |
| 501 | 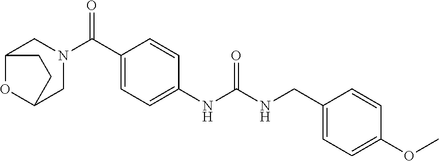 [SMILES: COc1ccc(CNC(=O)Nc2ccc(C(=O)N3CC4CCC(C3)O4)cc2)cc1] | 1-(4-(8-oxa-3- azabicyclo[3.2.1]octane-3- carbonyl)phenyl)-3-(4- methoxybenzyl)urea |
| 502 | 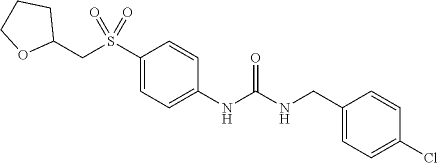 [SMILES: O=C(NCc1ccc(Cl)cc1)Nc1ccc(S(=O)(=O)CC2CCCO2)cc1] | 1-(4-chlorobenzyl)-3-(4- (((tetrahydrofuran-2- yl)methyl)sulfonyl)phenyl)urea |
| 503 | 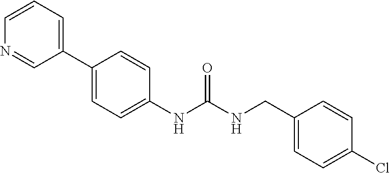 [SMILES: O=C(NCc1ccc(Cl)cc1)Nc1ccc(-c2cccnc2)cc1] | 1-(4-chlorobenzyl)-3-(4- (pyridin-3-yl)phenyl)urea |
| 504 | 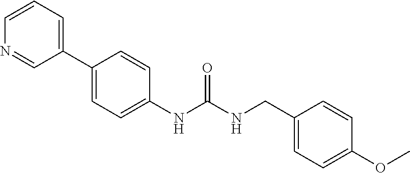 [SMILES: COc1ccc(CNC(=O)Nc2ccc(-c3cccnc3)cc2)cc1] | 1-(4-methoxybenzyl)-3-(4- (pyridin-3-yl)phenyl)urea |
| 505 | 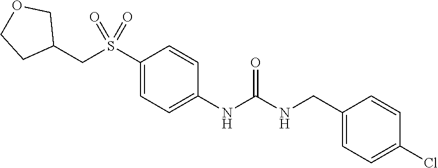 [SMILES: O=C(NCc1ccc(Cl)cc1)Nc1ccc(S(=O)(=O)CC2CCOC2)cc1] | 1-(4-chlorobenzyl)-3-(4- (((tetrahydrofuran-3- yl)methyl)sulfonyl)phenyl)urea |
| 506 | 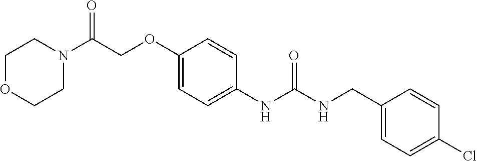 [SMILES: O=C(NCc1ccc(Cl)cc1)Nc1ccc(OCC(=O)N2CCOCC2)cc1] | 1-(4-chlorobenzyl)-3-(4-(2- morpholino-2- oxoethoxy)phenyl)urea |
| 507 | 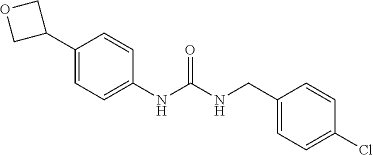 [SMILES: O=C(NCc1ccc(Cl)cc1)Nc1ccc(C2COC2)cc1] | 1-(4-chlorobenzyl)-3-(4- (oxetan-3-yl)phenyl)urea |
| 508 | 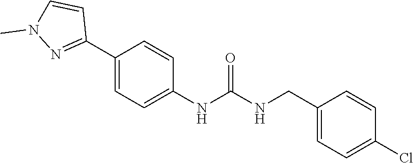 [SMILES: Cn1ccc(-c2ccc(NC(=O)NCc3ccc(Cl)cc3)cc2)n1] | 1-(4-chlorobenzyl)-3-(4-(1- methyl-1H-pyrazol-3- yl)phenyl)urea |
| 509 | 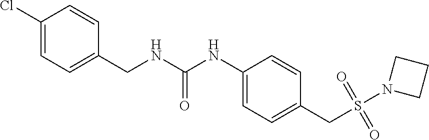 [SMILES: O=C(NCc1ccc(Cl)cc1)Nc1ccc(CS(=O)(=O)N2CCC2)cc1] | 1-(4-((azetidin-1- ylsulfonyl)methyl)phenyl)-3-(4- chlorobenzyl)urea |
| 510 | 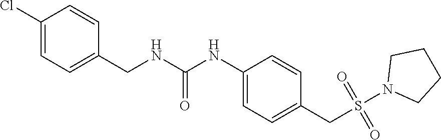 [SMILES: O=C(NCc1ccc(Cl)cc1)Nc1ccc(CS(=O)(=O)N2CCCC2)cc1] | 1-(4-chlorobenzyl)-3-(4- ((pyrrolidin-1- ylsulfonyl)methyl)phenyl)urea |
| 511 | 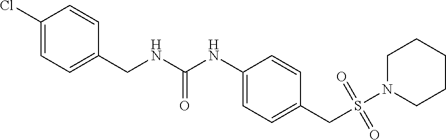 [SMILES: O=C(NCc1ccc(Cl)cc1)Nc1ccc(CS(=O)(=O)N2CCCCC2)cc1] | 1-(4-chlorobenzyl)-3-(4- ((piperidin-1- ylsulfonyl)methyl)phenyl)urea |
| 512 | 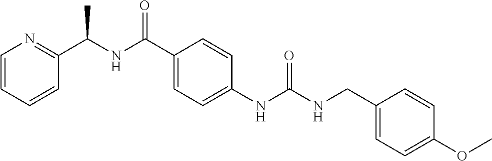 [SMILES: COc1ccc(CNC(=O)Nc2ccc(C(=O)N[C@H](C)c3ccccn3)cc2)cc1] | (R)-4-(3-(4- methoxybenzyl)ureido)-N-(1- (pyridin-2-yl)ethyl)benzamide |
| 513 | 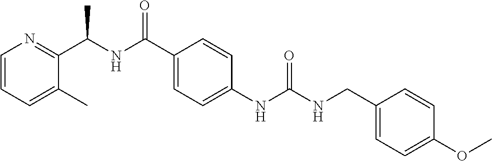 [SMILES: COc1ccc(CNC(=O)Nc2ccc(C(=O)N[C@H](C)c3ncccc3C)cc2)cc1] | (R)-4-(3-(4- methoxybenzyl)ureido)-N-(1- (3-methyipyridin-2- yl)ethyl)benzamide |
| 514 | 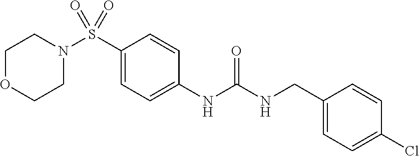 [SMILES: O=C(NCc1ccc(Cl)cc1)Nc1ccc(S(=O)(=O)N2CCOCC2)cc1] | 1-(4-chlorobenzyl)-3-(4- (morpholinosulfonyl)phenyl)urea |
| 515 | 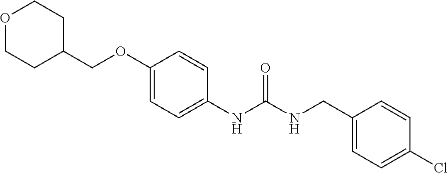 [SMILES: O=C(NCc1ccc(Cl)cc1)Nc1ccc(OCC2CCOCC2)cc1] | 1-(4-chlorobenzyl)-3-(4- ((tetrahydro-2H-pyran-4- yl)methoxy)phenyl)urea |
| 516 | 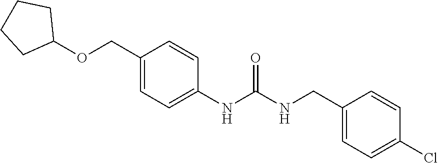 [SMILES: O=C(NCc1ccc(Cl)cc1)Nc1ccc(COC2CCCC2)cc1] | 1-(4-chlorobenzyl)-3-(4- ((cyclopentyloxy)methyl)phenyl) urea |
| 517 | 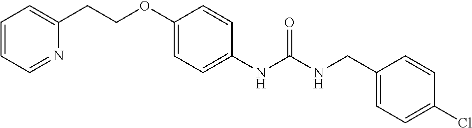 [SMILES: O=C(NCc1ccc(Cl)cc1)Nc1ccc(OCCc2ccccn2)cc1] | 1-(4-chlorobenzyl)-3-(4-(2- (pyridin-2- yl)ethoxy)phenyl)urea |
| 518 | 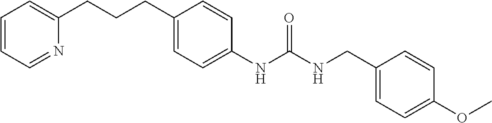 [SMILES: COc1ccc(CNC(=O)Nc2ccc(CCCc3ccccn3)cc2)cc1] | 1-(4-methoxybenzyl)-3-(4-(2- (pyridin-2- yl)ethoxy)phenyl)urea |
| 519 | 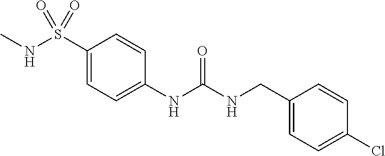 [SMILES: CNS(=O)(=O)c1ccc(NC(=O)NCc2ccc(Cl)cc2)cc1] | 4-(3-(4-chlorobenzyl)ureido)- N-methylbenzenesulfonamide |
| 520 | 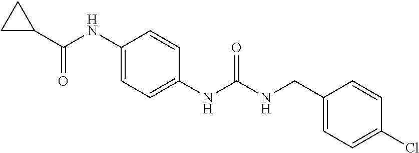 [SMILES: O=C(NCc1ccc(Cl)cc1)Nc1ccc(NC(=O)C2CC2)cc1] | N-(4-(3-(4- chlorobenzyl)ureido)phenyl) cyclopropanecarboxamide |
| 521 | 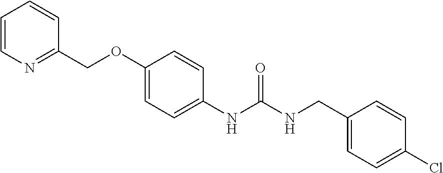 [SMILES: O=C(NCc1ccc(Cl)cc1)Nc1ccc(OCc2ccccn2)cc1] | 1-(4-chlorobenzyl)-3-(4- (pyridin-2- ylmethoxy)phenyl)urea |
| 522 | 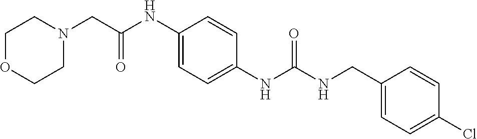 [SMILES: O=C(CN1CCOCC1)Nc1ccc(NC(=O)NCc2ccc(Cl)cc2)cc1] | N-(4-(3-(4- chlorobenzyl)ureido)phenyl)-2- morpholinoacetamide |
| 523 | 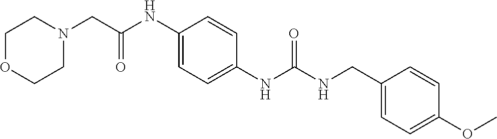 [SMILES: COc1ccc(CNC(=O)Nc2ccc(NC(=O)CN3CCOCC3)cc2)cc1] | N-(4-(3-(4- methoxybenzyl)ureido)phenyl)- 2-morpholinoacetamide |
| 524 | 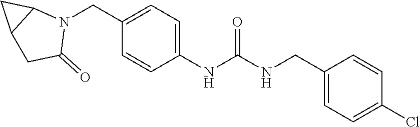 [SMILES: O=C(NCc1ccc(Cl)cc1)Nc1ccc(CN2C(=O)CC3CC32)cc1] | 1-(4-chlorobenzyl)-3-(4-((3- oxo-2-azabicyclo[3.1.0]hexan- 2-yl)methyl)phenyl)urea |
| 525 | 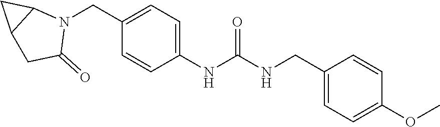 [SMILES: COc1ccc(CNC(=O)Nc2ccc(CN3C(=O)CC4CC43)cc2)cc1] | 1-(4-methoxybenzyl)-3-(4-((3- oxo-2-azabicyclo[3.1.0]hexan- 2-yl)methyl)phenyl)urea |
| 526 | 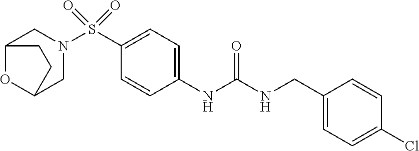 [SMILES: O=C(NCc1ccc(Cl)cc1)Nc1ccc(S(=O)(=O)N2CC3CCC(C2)O3)cc1] | 1-(4-((8-oxa-3- azabicyclo[3.2.1]octan-3- yl)sulfonyl)phenyl)-3-(4- chlorobenzyl)urea |
| 527 | 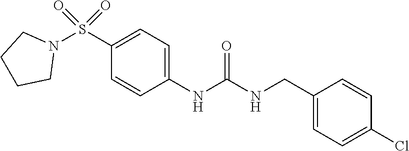 [SMILES: O=C(NCc1ccc(Cl)cc1)Nc1ccc(S(=O)(=O)N2CCCC2)cc1] | 1-(4-chlorobenzyl)-3-(4- (pyrrolidin-1- ylsulfonyl)phenyl)urea |
| 528 | 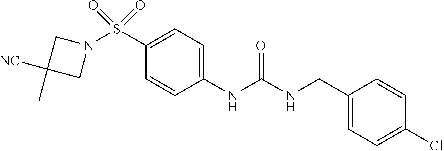 [SMILES: CC1(C#N)CN(S(=O)(=O)c2ccc(NC(=O)NCc3ccc(Cl)cc3)cc2)C1] | 1-(4-chlorobenzyl)-3-(4-((3- cyano-3-methylazetidin-1- yl)sulfonyl)phenyl)urea |
| 529 | 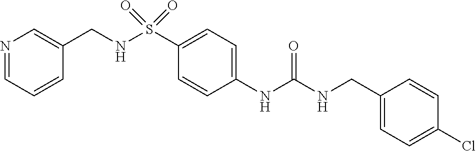 [SMILES: O=C(NCc1ccc(Cl)cc1)Nc1ccc(S(=O)(=O)NCc2cccnc2)cc1] | 4-(3-(4-chlorobenzyl)ureido)- N-(pyridin-3- ylmethyl)benzenesulfonamide |
| 530 | 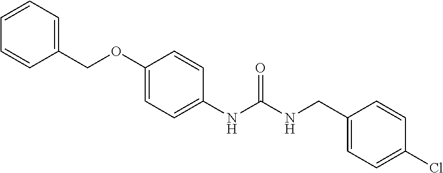 [SMILES: O=C(NCc1ccc(Cl)cc1)Nc1ccc(OCc2ccccc2)cc1] | 1-(4-(benzyloxy)phenyl)-3-(4- chlorobenzyl)urea |
| 531 | 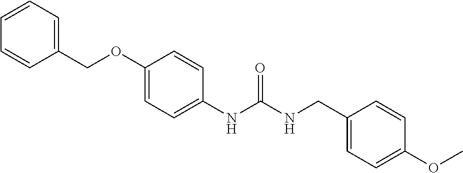 [SMILES: COc1ccc(CNC(=O)Nc2ccc(OCc3ccccc3)cc2)cc1] | 1-(4-(benzyloxy)phenyl)-3-(4- methoxybenzyl)urea |
| 532 | 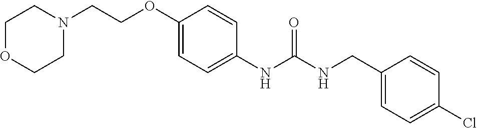 [SMILES: O=C(NCc1ccc(Cl)cc1)Nc1ccc(OCCN2CCOCC2)cc1] | 1-(4-chlorobenzyl)-3-(4-(2- morpholinoethoxy)phenyl)urea |
| 533 | 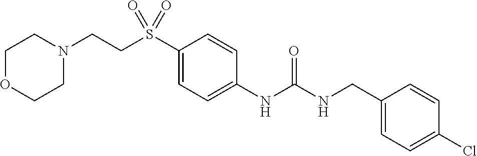 [SMILES: O=C(NCc1ccc(Cl)cc1)Nc1ccc(S(=O)(=O)CCN2CCOCC2)cc1] | 1-(4-chlorobenzyl)-3-(4-((2- morpholinoethyl)sulfonyl) phenyl)urea |
| 534 | 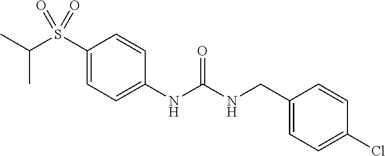 [SMILES: CC(C)S(=O)(=O)c1ccc(NC(=O)NCc2ccc(Cl)cc2)cc1] | 1-(4-chlorobenzyl)-3-(4- (isopropylsulfonyl)phenyl)urea |
| 535 | 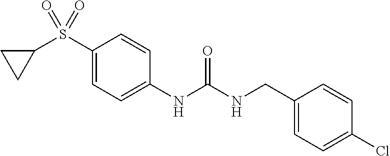 [SMILES: O=C(NCc1ccc(Cl)cc1)Nc1ccc(S(=O)(=O)C2CC2)cc1] | 1-(4-chlorobenzyl)-3-(4- (cyclopropylsulfonyl)phenyl)urea |
| 536 | 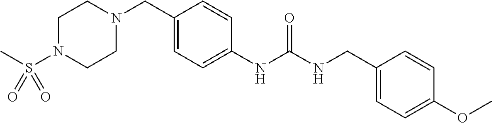 | 1-(4-methoxybenzyl)-3-(4-((4- (methylsulfonyl)piperazin-1- yl)methyl)phenyl)urea |
| 537 | 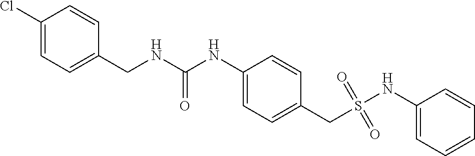 [SMILES: O=C(NCc1ccc(Cl)cc1)Nc1ccc(CS(=O)(=O)Nc2ccccc2)cc1] | 1-(4-(3-(4- chlorobenzyl)ureido)phenyl)-N- phenylmethanesulfonamide |
| 538 | 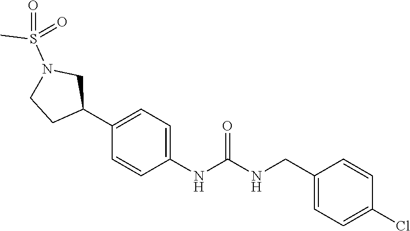 [SMILES: CS(=O)(=O)N1CC[C@H](c2ccc(NC(=O)NCc3ccc(Cl)cc3)cc2)C1] | (R)-1-(4-chlorobenzyl)-3-(4-(1- (methylsulfonyl)pyrrolidin-3- yl)phenyl)urea |
| 539 | 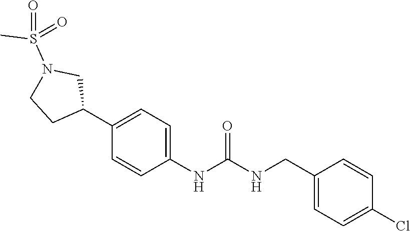 [SMILES: CS(=O)(=O)N1CC[C@@H](c2ccc(NC(=O)NCc3ccc(Cl)cc3)cc2)C1] | (S)-1-(4-chlorobenzyl)-3-(4-(1- (methylsulfonyl)pyrrolidin-3- yl)phenyl)urea |
| 540 | 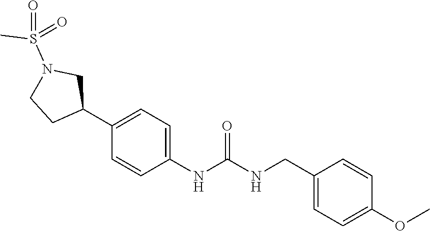 | (R)-1-(4-methoxybenzyl)-3-(4- (1-(methylsulfonyl)pyrrolidin- 3-yl)phenyl)urea |
| 541 | 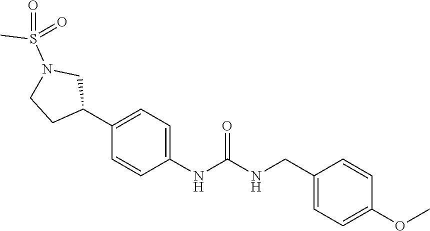 | (S)-1-(4-methoxybenzyl)-3-(4- (1-(methylsulfonyl)pyrrolidin- 3-yl)phenyl)urea |
| 542 | 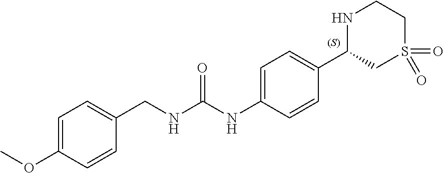 | (S)-1-(4-(1,1- dioxidothiomorpholin-3- yl)phenyl)-3-(4- methoxybenzyl)urea |
| 543 | 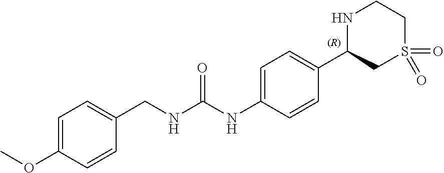 | (R)-1-(4-(1,1- dioxidothiomorpholin-3- yl)phenyl)-3-(4- methoxybenzyl)urea |
| 544 | 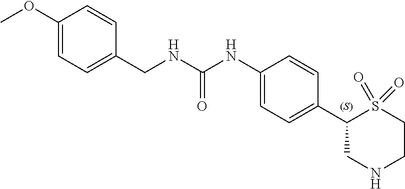 | (S)-1-(4-(1,1- dioxidothiomorpholin-2- yl)phenyl)-3-(4- methoxybenzyl)urea |
| 545 | 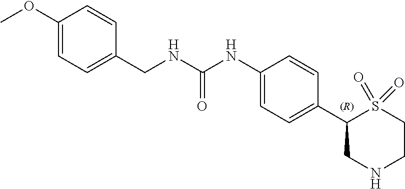 | (R)-1-(4-(1,1- dioxidothiomorpholin-2- yl)phenyl)-3-(4- methoxybenzyl)urea |
| 546 | 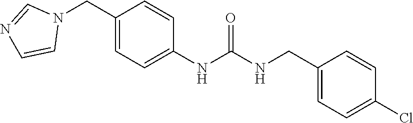 [SMILES: O=C(NCc1ccc(Cl)cc1)Nc1ccc(Cn2ccnc2)cc1] | 1-(4-((1H-1,2,4-triazol-1- yl)methyl)phenyl)-3-(4- chlorobenzyl)urea |
| 547 | 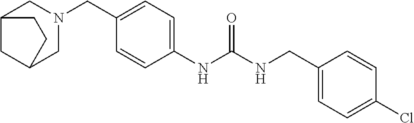 [SMILES: O=C(NCc1ccc(Cl)cc1)Nc1ccc(CN2CC3CCC(C3)C2)cc1] | 1-(4-((8-oxa-3- azabicyclo[3.2.1]octan-3- yl)methyl)phenyl)-3-(4- chlorobenzyl)urea |
| 548 | 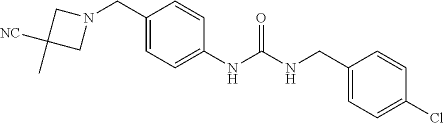 [SMILES: CC1(C#N)CN(Cc2ccc(NC(=O)NCc3ccc(Cl)cc3)cc2)C1] | 1-(4-chlorobenzyl)-3-(4-((3- cyano-3-methylazetidin-1- yl)methyl)phenyl)urea |
| 549 | 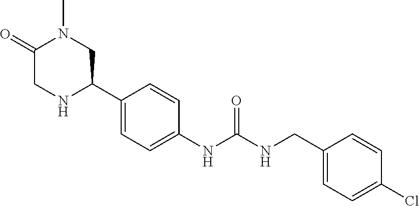 [SMILES: CN1C[C@@H](c2ccc(NC(=O)NCc3ccc(Cl)cc3)cc2)NCC1=O] | (R)-1-(4-chlorobenzyl)-3-(4-(4- methyl-5-oxopiperazin-2- yl)phenyl)urea |
| 550 | 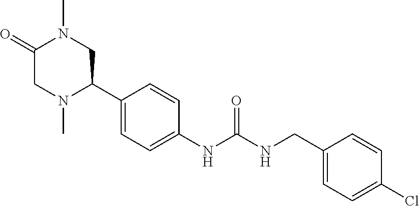 [SMILES: CN1C[C@@H](c2ccc(NC(=O)NCc3ccc(Cl)cc3)cc2)N(C)CC1=O] | (R)-1-(4-chlorobenzyl)-3-(4- (1,4-dimethyl-5-oxopiperazin- 2-yl)phenyl)urea |
| 551 | 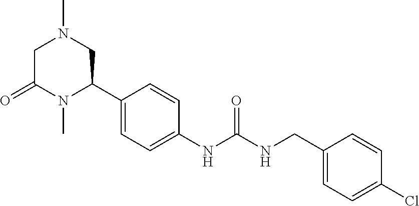 [SMILES: CN1CC(=O)N(C)[C@H](c2ccc(NC(=O)NCc3ccc(Cl)cc3)cc2)C1] | (R)-1-(4-chlorobenzyl)-3-(4- (1,4-dimethyl-6-oxopiperazin- 2-yl)phenyl)urea |
| 552 | 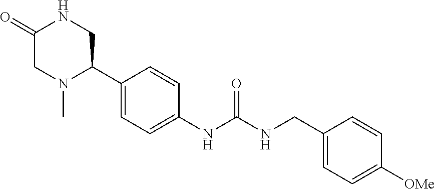 [SMILES: COc1ccc(CNC(=O)Nc2ccc([C@@H]3CNC(=O)CN3C)cc2)cc1] | (R)-1-(4-methoxybenzyl)-3-(4- (1-methyl-5-oxopiperazin-2- yl)phenyl)urea |

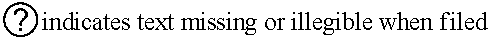

In some variations, any of the compounds described herein, such as a compound of Formula (II), (I), (I-G), (I) (I-A), (I-A1), (I-A2), (I-A3), (I-A4), (I-B), (I-B1), (I-1B2), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1), or any variation thereof, or a compound of Table 1 may be deuterated (e.g., a hydrogen atom is replaced by a deuterium atom). In some of these variations, the compound is deuterated at a single site. In other variations, the compound is deuterated at multiple sites. Deuterated compounds can be prepared from deuterated starting materials in a manner similar to the preparation of the corresponding non-deuterated compounds. Hydrogen atoms may also be replaced with deuterium atoms using other method known in the art.

Any formula given herein, such as Formula (II), (I-G), (I) (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1), is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of Table 1 is depicted with a particular stereochemical configuration, also provided herein is any alternative stereochemical configuration of the compound, as well as a mixture of stereoisomers of the compound in any ratio. For example, where a compound of Table 1 has a stereocenter that is in an “S” stereochemical configuration, also provided herein is enantiomer of the compound wherein that stereocenter is in an “R” stereochemical configuration. Likewise, when a compound of Table 1 has a stereocenter that is in an “R” configuration, also provided herein is enantiomer of the compound in an “S” stereochemical configuration. Also provided are mixtures of the compound with both the “S” and the “R” stereochemical configuration. Additionally, if a compound of Table 1 has two or more stereocenters, also provided are any enantiomer or diastereomer of the compound. For example, if a compound of Table 1 contains a first stereocenter and a second stereocenter with “R” and “R” stereochemical configurations, respectively, also provided are stereoisomers of the compound having first and second stereocenters with “S” and “S” stereochemical configurations, respectively, “S” and “R” stereochemical configurations, respectively, and “R” and “S” stereochemical configurations, respectively. If a compound of Table 1 contains a first stereocenter and a second stereocenter with “S” and “S” stereochemical configurations, respectively, also provided are stereoisomers of the compound having first and second stereocenters with “R” and “R” stereochemical configurations, respectively, “S” and “R” stereochemical configurations, respectively, and “R” and “S” stereochemical configurations, respectively. If a compound of Table 1 contains a first stereocenter and a second stereocenter with “S” and “R” stereochemical configurations, respectively, also provided are stereoisomers of the compound having first and second stereocenters with “R” and “S” stereochemical configurations, respectively, “R” and “R” stereochemical configurations, respectively, and “S” and “S” stereochemical configurations, respectively. Similarly, if a compound of Table 1 contains a first stereocenter and a second stereocenter with “R” and “S” stereochemical configurations, respectively, also provided are stereoisomers of the compound having first and second stereocenters with “S” and “R” stereochemical configurations, respectively, “R” and “R” stereochemical configurations, respectively, and “S” and “S” stereochemical configurations, respectively. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Representative examples of compounds detailed herein, including intermediates and final compounds, are depicted in the tables and elsewhere herein. It is understood that in one aspect, any of the compounds may be used in the methods detailed herein, including, where applicable, intermediate compounds that may be isolated and administered to an individual or subject.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

In one variation, the compounds herein are synthetic compounds prepared for administration to an individual or subject. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of R¹, R², R³, R⁴, R⁵, R⁶, Z¹, Z², Z³, Z⁴, Z⁵, Z⁶, Z⁷, Z⁸, Z⁹, Z¹⁰, Z¹¹, Z¹², Z¹³, Z¹⁴, R^{a}, R^{b}, R^{c}, R^{d}, R^{e}, R^{f}, R^{g}, R^{h}, R^{j}, R^{k}, R^{m}, Rⁿ, R^{o}, R^{p}, R^{q}, R^{r}, R^{s}, R^{t}, R^{x}, R^{y}, R^{z}, R^{A}, R^{B}, R^{C}, m, n, p, and q provided herein can be combined with every other variation or embodiment of R¹, R², R³, R⁴, R⁵, R⁶, Z¹, Z², Z³, Z⁴, Z⁵, Z⁶, Z⁷, Z⁸, Z⁹, Z¹⁰, Z¹¹, Z¹, Z¹, Z¹⁴, R^{a}, R^{c}, R^{d}, R^{e}, R^{f}, R^{g}, R^{h}, R^{j}, R^{k}, R^{m}, Rⁿ, R^{o}, R^{p}, R^{q}, R^{r}, R^{s}, R^{t}, R^{x}, R^{y}, R^{z}, R^{A}, R^{B}, R^{C}, m, n, p, and q, the same as if each combination had been individually and specifically described.

Other embodiments will be apparent to those skilled in the art from the following detailed description.

As used herein, when any variable occurs more than one time in a chemical formula, its definition on each occurrence is independent of its definition at every other occurrence.

Formula (II) includes all subformulae thereof. For example, Formula (II) includes compounds of Formula (I-G), (I) (I-A), (I-A1), (I-A2), (I-A3), (I-A4), (I-B), (I-B1), (I-B2), (I-B33), (I-C), (I-C1), (I-C2), (I-C3), (I-C4), (I-D), (I-D1), (I-D32), (I-D33), (I-D34), (I-D35), (I-D6), (I-D7), (I-E), (I-F), (II-A), and (II-A1).

The names for compounds 1-552 provided herein, as shown in Table 1 and Examples 1-16, are provided by ChemInnovation's Chem 4d software version 7.5.0.0. The names for the intermediates 1.1-10.0 as shown in Examples A-MM are provided by ChemBioDraw Professional 15.0. One of skilled in the art would understand that the compounds may be named or identified using various commonly recognized nomenclature systems and symbols. By way of example, the compounds may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

### Compositions

Also provided are compositions, such as pharmaceutical compositions, that include a compound disclosed and/or described herein and one or more additional medicinal agents, pharmaceutical agents, adjuvants, carriers, excipients, and the like. Suitable medicinal and pharmaceutical agents include those described herein. In some embodiments, the pharmaceutical composition includes a pharmaceutically acceptable excipient or adjuvant and at least one chemical entity as described herein. Examples of pharmaceutically acceptable excipients include, but are not limited to, mannitol, lactose, starch, magnesium stearate, sodium saccharine, talcum, cellulose, sodium crosscarmellose, glucose, gelatin, sucrose, and magnesium carbonate. In some embodiments, provided are compositions, such as pharmaceutical compositions that contain one or more compounds described herein, or a pharmaceutically acceptable salt thereof.

In some embodiments, provided is a pharmaceutically acceptable composition comprising a compound of Formula (II), (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D32), (I-D33), (I-D4), (I-D5), (I-D6), (I-D7), (I-E), (I-F), or (II-A), or a compound of Table 1, or a pharmaceutically acceptable salt thereof. In some aspects, a composition may contain a synthetic intermediate that may be used in the preparation of a compound described herein. The compositions described herein may contain any other suitable active or inactive agents.

Any of the compositions described herein may be sterile or contain components that are sterile. Sterilization can be achieved by methods known in the art. Any of the compositions described herein may contain one or more compounds or conjugates that are substantially pure.

Also provided are packaged pharmaceutical compositions, comprising a pharmaceutical composition as described herein and instructions for using the composition to treat a patient suffering from a disease or condition described herein.

### Methods of Use

Compounds and compositions detailed herein, such as a pharmaceutical composition comprising a compound of any formula provided herein, or a pharmaceutically acceptable salt thereof, and a pharmaceutically acceptable carrier or excipient, may be used in methods of administration and treatment as provided herein.

Without being bound by theory, the compounds and pharmaceutical compositions disclosed herein are believed to act by modulating nicotinamide phosphoribosyltransferase (NAMPT). In some embodiments, the compounds and pharmaceutical compositions disclosed herein are activators of NAMPT. In some embodiments, provided are methods of treating a disease or condition mediated by NAMPT activity in an individual or subject, comprising administering to the individual or subject in need thereof a compound of Formula (II), (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), or (II-A), or a compound of Table 1, or a pharmaceutically acceptable salt thereof. In some embodiments, provided are methods of treating cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder in an individual or subject, comprising administering to the individual or subject in need thereof a compound of Formula (II), (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), or (II-A), or a compound of Table 1, or a pharmaceutically acceptable salt thereof.

Also provided herein is the use of a compound of Formula (II), (I-G), (I), (I-A), (I-A1), (I-A2), (I-A3), (I-A4), (I-B), (I-B1), (I-B2), (I-B3), (I-C), (I-C1), (I-C2), (I-C3), (I-C4), (I-D), (I-D1), (I-D2), (I-D3), (I-D4), (I-D5), (I-D6), (I-D7), (I-E), (I-F), or (II-A), or a compound of Table 1, or a pharmaceutically acceptable salt thereof in the manufacture of a medicament for treatment of a disease or condition mediated by NAMPT activity in a subject. In some aspects, provided is a compound or composition as described herein for use in a method of treatment of the human or animal body by therapy. In some embodiments, provided herein are compounds of Formula (II), (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D32), (I-D3), (I-D4), (I-D5), (I-D6), (I-D7), (I-E), (I-F), or (II-A), or a compound of Table 1, or a pharmaceutically acceptable salt thereof, for use in a method of treatment of the human or animal body by therapy. In some embodiments, provided herein are compounds of Formula (II), (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), or (II-A), or a compound of Table 1, or a pharmaceutically acceptable salt thereof, for use in treating a disease or condition mediated by NAMPT activity. In some embodiments, the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder.

Also provided herein are compositions (including pharmaceutical compositions) as described herein for the use in treating, preventing, and/or delaying the onset and/or development of a disease described herein and other methods described herein. In certain embodiments, the composition comprises a pharmaceutical formulation which is present in a unit dosage form.

In some embodiments, the subject is a mammal. In some embodiments, the subject is a mouse, rat, dog, cat, rabbit, pig, sheep, horse, cow, or human. In some embodiments, the subject is a human.

There are numerous conditions in which small molecule-mediated stimulation of NAMPT activity that boosts NAD+ levels would potentially be clinically beneficial (Stromland et al., Biochem Soc Trans. 2019, 47(1):119-130; Ralto et al., Nat Rev Nephrol. 2019; Fang et al., Trends Mol Med. 2017, 23(10):899-916; Yoshino et al., Cell Metab. 2011, 14(4):528-36; Yang and Sauve, Biochim Biophys Acta. 2016, 1864:1787-1800; Verdin, Science. 2015, 350(6265):1208-13). These conditions include, but are not limited to, cardiac diseases, chemotherapy induced tissue damage, renal diseases, metabolic diseases, muscular diseases, neurological diseases and injuries, diseases caused by impaired stem cell function, and DNA damage and primary mitochondrial disorders. In some embodiments, the disease or condition mediated by NAMPT activity is a cardiac disease, chemotherapy induced tissue damage, a renal disease, a metabolic disease, a muscular disease, a neurological disease or injury, a disease caused by impaired stem cell function, or DNA damage and primary mitochondrial disorder.

Cardiac diseases. In various preclinical models of heart failure NAD as well as NAMPT levels are decreased. In these models, cardiac function can be rescued, either by restoring NAD via oral supplementation or overexpression of NAMPT (Diguet et al, Circulation. 2018, 137:2256-2273; Zheng et al., Clin Sci (Lond). 2019, 133(13):1505-1521; Smyrnias et al., J Am Coll Cardiol. 2019, 73(14):1795-1806). Thus, increasing the catalytic efficiency of NAMPT with a small molecule activator to compensate for the decreased protein levels is a promising strategy to treat various forms of heart failure.

Chemotherapy induced tissue damage. Use of chemotherapy regimens frequently is limited by toxicity to healthy tissues and severe oxidative stress is thought to play a major role. NAD boosting has been shown to trigger a strong anti-oxidant response. Therefore, NAMPT activators are considered broadly useful in various settings of chemotherapy to prevent reversible and irreversible secondary pathologies. Examples are anthracycline and trastuzumab cardiotoxicity, cisplatin induced kidney injury, peripheral neuropathies induced by cisplatin, paclitaxel, vincristine and other agents. Neuroprotection by NAMPT activation is also useful in treating/preventing chemotherapy associated cognitive (“chemo brain”), which is caused by destruction of healthy nerve tissue, both during active treatment and long after treatment has been halted. For instance, see Zheng et al., Clin Sci (Lond). 2019, 133(13):1505-1521.

Renal diseases. Renal diseases are highly prevalent and an area of urgent unmet medical need. In approximately 3% of hospitalized patients, acute kidney injury (AKI) is diagnosed. A subset of patients will progress to chronic kidney disease that may require long-term dialysis or kidney transplantation. A key feature of kidney dysfunction is a decrease in the activities of SIRT1 and SIRT3, characterized by a reduction of the sirtuin substrate NAD, primarily due to impairment of de novo NAD+ synthesis. NAMPT is robustly expressed during kidney injury, thus small molecule activation with NAMPT is considered an effective measure to prevent AKI. Similarly, kidney mesangial cell hypertrophy exhibits depletion of NAD+, and restoration of intracellular NAD+ levels is considered efficacious. For instance, see Poyan Mehr et al., Nat Med. 2018, September; 24(9): 1351-9.

Metabolic disease. NAD+ boosting improves insulin sensitivity, dyslipidemia, mitochondrial function in metabolic disease and protects from/improves non-alcoholic and alcoholic steatohepatitis in preclinical models. More than 3 million people per year in the U.S. alone are diagnosed with non-alcoholic steatohepatitis and it is one of the leading causes of liver transplantation. See Guarino and Dufour, Metabolites. 2019, Sep. 10; 9(9), pii: E180; Yoshino et al., Cell Metab. 2011, 14(4):528-36.

Muscular diseases. Preclinical data has suggested that NAD+ boosting strategies could alleviate skeletal muscle dysfunction in a number of conditions, including Duchenne's muscular dystrophy, and age-related sarcopenia. See Zhang et al., Clin Sci (Lond). 2019, 133(13):1505-1521; Mohamed et al., Aging (Albany N.Y.). 2014, 6(10):820-34; Ryu et al., Sci Transl Med. 2016, 8(361):361ra139.

Neurological diseases and injuries. Repletion of NAD by means of NAMPT activation is neuroprotective and of therapeutic benefit in a wide range of preclinical models of neurological diseases and injuries, including age-related cognitive decline, glaucoma, ischemic stroke, and ALS. See Johnson et al., NPJ Aging Mech Dis. 2018, 4:10; Harlan et al., J Biol Chem. 2016, 291(20):10836-46; Zhao et al., Stroke. 2015, July; 46(7):1966-74; Williams et al., Front Neurosci. 2017, Apr. 25; 11:232.

Diseases caused by impaired stem cell function. NAD boosting promotes stem cell activation and hematopoiesis and is useful in accelerating the expansion of stem cell populations following a stem cell transplant. See Pi et al., Aging (Albany N.Y.). 2019, 11(11):3505-3522.

DNA damage disorders and primary mitochondrial disorders. NAMPT activators will also be useful in the treatment of DNA damage disorders which are associated with an accelerated aging phenotype, such as Xeroderma pigmentosum, Cockayne syndrome, and Ataxia telangiectasia. Similarly, there are several primary mitochondrial disorders with shared symptoms and manifestations for which NAD boosting via NAMPT activation may be a suitable therapeutic intervention. See Fang et al, Cell. 2014, 157(4):882-896; Khan et al, EMBO Mol Med. 2014, June; 6(6):721-31; Cerutti et al., Cell Metab. 2014, 19(6):1042-9.

Provided in some embodiments are methods of treating a disease or condition mediated by NAMPT activity in a subject in need thereof, comprising administering to the individual or subject in need thereof a compound of Formula (II), (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), or (II-A), or a compound of Table 1, or a pharmaceutically acceptable salt thereof, wherein the disease or condition is selected from the group consisting of cardiac diseases, chemotherapy induced tissue damage, renal diseases, metabolic diseases, muscular diseases, neurological diseases and injuries, diseases caused by impaired stem cell function, and DNA damage and primary mitochondrial disorders.

Additional applications of small molecule NAMPT activators are provided in Table 2.

TABLE 2

| | |
|---|---|
| Cancer and | Anthracycline and trastuzumab cardiotoxicity |
| Chemotherapy | Proteasome inhibitor cardiotoxicity |
| induced tissue | Cisplatin induced kidney injury |
| damage | Prevention/treatment of cognitive dysfunction resulting |
| | from chemotherapy (“chemo brain”) |
| | Chemotherapy induced impairment of hematopoiesis and |
| | myelosuppression |
| | Cachexia of cancer |
| | Chemoprevention of non-melanoma skin cancer in high |
| | risk patients |
| | chemoprevention of hepatocellular carcinoma |
| Cardiovascular | Heart failure with reduced ejection fraction |
| diseases | Heart failure with preserved ejection fraction |
| | Hypertrophic cardiomyopathy |
| | Cardiac arrhythmias |
| | Duchenne Muscular Dystrophy-related cardiac |
| | dysfunction |
| | Cardiac dysfunction associated with Scleroderma, |
| | Lupus, Mitochondrial Disorders, Kawasaki Disease |
| | Hypertension |
| | Myocardial Infarction |
| Renal diseases | Acute kidney injury including nephropathy following |
| | major surgeries including cardiac and vascular |
| | surgeries |
| | Acute kidney injury following hypotension, hemorrhagic |
| | shock, or cardiac arrest |
| | Acute kidney injury following exposure to contrast |
| | imaging agents used for MRI, CT scans, or other |
| | imaging modalities, particularly in the context of |
| | diabetes |
| | Chronic kidney disease |
| | Glomerular nephritis |
| | Kidney mesangial cell hypertrophy |
| | Arterial venous fistula maturation |
| Chronic | Chronic obstructive pulmonary disease |
| inflammatory | Asthma |
| and fibrotic | Scleroderma |
| diseases | Dermatomyositis |
| | Lupus erythematosus |
| | Rheumatoid arthritis and spondyloarthropathy |
| | Juvenile idiopathic arthritis |
| | Crohn's disease |
| | Inflammatory Bowel Disease |
| | Eczema |
| | Psoriasis and psoriatic arthritis |
| | Idiopathic pulmonary fibrosis |
| Vascular | Arterial and venous thrombosis |
| diseases | Ischemic Stroke |
| | Arteriosclerosis |
| Metabolic | Obesity |
| dysfunction | Diabetes |
| | Metabolic Syndrome |
| | Alcoholic steatohepatitis |
| | Non-alcoholic steatohepatitis |
| | Dyslipidemia |
| | Diabetic neuropathy |
| | Diabetic gastroparesis |
| Muscular | Muscular dystrophies, including: Duchenne, Becker's, |
| diseases | Congenital, Distal, Emery-Dreifuss', Facio-scapulo- |
| | humeral, Limb-girdle, myotonic, and oculopharyngeal |
| | Sarcopenia |
| | Frailty |
| | Polymyositis |
| | Muscle stem cell senescence developed in the context |
| | of nutritional deficiencies |
| | Non-mitochondrial myopathies such as inherited |
| | myopathies, myotonia, congenital myopathies selected |
| | from nemaline myopathy, multi/minicore myopathy, |
| | centronuclear myopathy and metabolic myopathies, |
| | inflammatory myopathies |
| Neurological | Depression |
| diseases | Frontotemporal dementia |
| and injuries | Multiple sclerosis |
| | Amyotrophic lateral sclerosis |
| | Peripheral neuropathy due to diabetes, chemotherapy |
| | Alzheimer's disease |
| | Parkinson's disease |
| | Huntington's Disease |
| | Spinal muscular atrophy |
| | Spinocerebellar ataxias |
| | Spastic paraplegias |
| | Glaucoma |
| | Age-related macular degeneration |
| | Age-related cognitive decline |
| | Noise induced and age-related hearing loss |
| | Ischemic stroke |
| | Traumatic brain injury |
| | Neonatal nerve damage |
| | Optic nerve injury |
| | Spinal cord injuries |
| | Peripheral neuropathies or tissue inflammation induced |
| | by cisplatin, paclitaxel, vincristine, other chemo- |
| | therapeutic agents, or radiation. |
| | Peripheral neuropathies (length and non-length |
| | dependent) affecting motor, sensory, or autonomic |
| | nerves, arising from: diabetes, impaired glucose |
| | tolerance, hypertension, infection, trauma, autoimmune |
| | disorders, vasculitis, arteriosclerosis, vitamin |
| | deficiencies (particularly B6 and B12), alcoholism, |
| | liver or kidney disease, or exposure to toxins |
| DNA damage | Xeroderma pigmentosum |
| disorders and | Cockayne syndrome |
| Primary | Ataxia telangiectasia |
| Mitochondrial | MEGDEL syndrome |
| Disorders | Charcot-Marie-Tooth type 2 |
| | Primary Mitochondrial Diseases (Disorders) including |
| | NARP, MELAS, Chronic Progressive External |
| | Ophthalmoplegia, Leigh's disease, Leber's Hereditary |
| | Optic Neuropathy, MERRF, Barth Syndrome, Luft |
| | Disease, Kearns Sayre Syndrome, Autosomal dominant |
| | optic atrophy |
| | Friedreich's ataxia |
| | Werner syndrome |
| General | Tissue repair following physical trauma, hemorrhagic |
| | shock, tissue grafting, organ transplant including |
| | heart, lung, liver, and kidney |
| | Stem cell therapies, including hematopoietic stem cell |
| | transfer, allogenic mesenchymal stem therapy for acute |
| | graft-vs-host disease, limbal stem cell deficiency due to |
| | genetic or acquired conditions that compromise normal |
| | turnover of the corneal epithelium |

In some embodiments, the disease or condition mediated by NAMPT activity is cancer and chemotherapy-induced tissue damage, a cardiovascular disease, a renal disease, chronic inflammatory and fibrotic disease, a vascular disease, metabolic dysfunction, a muscular disease, a neurological disease or injury, or a DNA damage disorder or primary mitochondrial disorder. Provided in some embodiments are methods of treating a disease or condition mediated by NAMPT activity in a subject in need thereof, comprising administering to the individual or subject in need thereof a compound of Formula (II), (I-G), (I), (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), or (II-A), or a compound of Table 1, or a pharmaceutically acceptable salt thereof. In some embodiments, the disease or condition is cancer or chemotherapy induced tissue damage, a cardiovascular disease, a renal disease, a chronic inflammatory or fibrotic disease, a vascular disease, metabolic dysfunction, a muscular disease, a neurological disease or injury, a DNA damage disorder or Primary Mitochondrial Disorder, including any of the diseases listed in Table 2.

### Permeability

Membrane permeability is a key property in small molecule drug design, especially for compounds that have intracellular targets, as their efficacy highly depends on their ability to cross the membrane. The efficacy of a drug can depend on the ability of the drug to reach the intended site of action. Drug absorption is the movement of a drug into the bloodstream. Many factors influence this process, including a drug's physicochemical properties, formulation, and route of administration. Generally, for oral treatment, the drug needs to be introduced via the intestinal pathway to blood. For other routes, like intravenous therapy, intramuscular injection, and enteral nutrition, absorption is more straightforward to blood. No matter what kind of administration routes, drugs must be dissolved and absorbed for therapeutic effects. By adjusting factors that affect absorption, the pharmacokinetic (PK) profile of a drug can be changed. A drug's permeability across biological membranes is a key factor that influences the absorption and distribution. This is because if a drug wants to reach to the systemic circulation, it needs to cross several semipermeable cell membranes firstly. Drugs may cross cell membranes by passive diffusion, facilitated passive diffusion, active transport, and pinocytosis. The drug's physicochemical properties (such as size and lipophilicity), as well as membrane-based efflux mechanisms, can lead to poor permeability.

For orally administered drugs, most absorption occurs in the small intestine. Therefore, drugs that are poorly absorbed by and/or actively effluxed out of the small intestine would have a low likelihood of actually reaching the intended site of action. This low likelihood of reaching the intended site of action would thereby greatly diminish the efficacy of the drug, requiring significantly higher and potentially unrealistic dosages compared to dosages that would be anticipated by in vitro on-target potency assays. Conversely, drugs that are readily absorbed and/or have a reduced amount of active efflux from the small intestine would likely require lower dosages to be administered than similar or even more “potent” drugs that are poorly absorbed. Accordingly, the ability of a drug to be absorbed by and the amount of efflux that occurs within the small intestine is an important consideration for the development of any orally administered drug.

There are a wide variety of in vitro methods to assess the permeability of drugs and predict their in vivo absorption. One such method is the Caco-2 permeability assay. The Caco-2 cell line is derived from a human colon carcinoma and has many characteristics that resemble intestinal epithelial cells. Caco-2 permeability assay is a good way to investigate human intestinal permeability and drug efflux. Monolayers of the Caco-2 cell line have been recognized as an accurate in vitro model of human small intestinal drug absorption. Even though the cell line was isolated from a human colon adenocarcinoma, differentiated Caco-2 cells resemble enterocytes (small intestinal absorptive cells) in that Caco-2 cells form functional tight junctions, apical and basolateral domains, and brush border cytoskeleton. Caco-2 permeability assay measures the rate of transporting of a compound across the Caco-2 cell and assesses transport in both directions. The in vitro apparent permeability (Pₐₐₚ) of a drug for Caco-2 cells in the apical to basolateral direction has been shown to correlate with in vivo oral absorption in humans, both in that drugs with poor Caco-2 cell permeability have poor small intestinal drug absorption in vivo and in that drugs with high or complete Caco-2 cell permeability have high small intestinal drug absorption in vivo (Artursson, et al., Biochem Biophys Res Comm, 1991, 3(29): 880-885). Typically, drugs that are completely absorbed in vivo have a permeability coefficient greater than 1×10⁻⁶ cm/second, and drugs that are poorly absorbed have a permeability coefficient less than 1×10-7 cm/second in the apical to basolateral direction in Caco-2 cells.

Additionally, Caco-2 cells have been used to identify and quantify levels of active efflux for a drug. Active efflux of a drug can be determined by calculating the ratio of Pₐₐₚ in the basolateral to apical direction and the Pₐₐₚ in the apical to basolateral direction. Typically, the lower the ratio, the greater the ability of the drug to reach the intended site of action, and the greater the ability of the drug to reach the intended site of action, the greater potential efficacy of the drug.

Compounds provided herein are suitable for oral administration as measured by their permeability characteristics as evaluated by the Caco-2 cellular model. Compounds described herein have been demonstrated to have improved permeability, as described in Biological Example 2 herein.

### Dosages

The compounds and compositions disclosed and/or described herein are administered at a therapeutically effective dosage, e.g., a dosage sufficient to provide treatment for the disease state. While human dosage levels have yet to be optimized for the chemical entities described herein, generally, a daily dose ranges from about 0.01 to 100 mg/kg of body weight; in some embodiments, from about 0.05 to 10.0 mg/kg of body weight, and in some embodiments, from about 0.10 to 1.4 mg/kg of body weight. Thus, for administration to a 70 kg person, in some embodiments, the dosage range would be about from 0.7 to 7000 mg per day; in some embodiments, about from 3.5 to 700.0 mg per day, and in some embodiments, about from 7 to 100.0 mg per day. The amount of the chemical entity administered will be dependent, for example, on the subject and disease state being treated, the severity of the affliction, the manner and schedule of administration and the judgment of the prescribing physician. For example, an exemplary dosage range for oral administration is from about 5 mg to about 500 mg per day, and an exemplary intravenous administration dosage is from about 5 mg to about 500 mg per day, each depending upon the compound pharmacokinetics.

A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

Administration of the compounds and compositions disclosed and/or described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

The compounds disclosed and/or described herein can be administered either alone or in combination with one or more conventional pharmaceutical carriers or excipients (e.g., mannitol, lactose, starch, magnesium stearate, sodium saccharine, talcum, cellulose, sodium crosscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pa.

In some embodiments, the compositions will take the form of a pill or tablet and thus the composition may contain, along with a compounds disclosed and/or described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

Liquid pharmaceutically administrable compositions can, for example, be prepared by dissolving, dispersing or suspending etc. a compound disclosed and/or described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compound contained in such parenteral compositions depends, for example, on the physical nature of the compound, the activity of the compound and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound disclosed and/or described herein in solution.

Pharmaceutical compositions of the compounds disclosed and/or described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns.

In addition, pharmaceutical compositions can include a compound disclosed and/or described herein and one or more additional medicinal agents, pharmaceutical agents, adjuvants, and the like. Suitable medicinal and pharmaceutical agents include those described herein.

### Kits

Also provided are articles of manufacture and kits containing any of the compounds or pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing a compound or composition described herein and instructions for use. The kits may contain instructions for use in the treatment of a heart disease in an individual or subject in need thereof. A kit may additionally contain any materials or equipment that may be used in the administration of the compound or composition, such as vials, syringes, or IV bags. A kit may also contain sterile packaging.

### Combinations

The compounds and compositions described and/or disclosed herein may be administered alone or in combination with other therapies and/or therapeutic agents useful in the treatment of the aforementioned disorders, diseases, or conditions.

### ENUMERATED EMBODIMENTS

The following enumerated embodiments are representative of some aspects of the invention.

1. A compound of Formula (I)

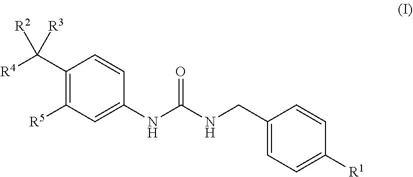

or a pharmaceutically acceptable salt thereof,
wherein:
R¹ is halo or methoxy;
R² is hydrogen or C₁-C₆ alkyl or is taken together with Z⁴ and the intervening atoms to form a 4-6 membered heterocycloalkyl or heterocycloalkenyl ring
R³ is hydrogen or C₁-C₆ alkyl;
R⁴ is
   a) Z¹NR^{a}C(O)—,
   b) Z²C(O)NR^{b}—,
   c) Z³(CR^{c}R^{d})ₘNR^{e}—,
   d) Z⁴S(O)₂(CH₂)ₙ—,
   e) Z⁵OC(O)—,
   f) NR^{f}R^{g}C(O)—,
   g) 5- to 10-membered heteroaryl optionally substituted with one or more independently selected C₁-C₆ alkyl substituents, or
   h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein
      R^{a} and R^{e} are each independently hydrogen or C₁-C₆ alkyl;
      R^{b} is hydrogen or C₁-C₆ alkyl or is taken together with R⁵ and the intervening atoms to form a 5- to 6-membered heterocycloalkyl or heterocycloalkenyl ring;
      R^{c} and R^{d} are each independently hydrogen or C₁-C₆ alkyl, or R^{c} and R^{d} together with the carbon to which they are attached form a C₃-C₆ cycloalkyl;
      R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl;
      each R^{h} is independently —C₁-C₆ alkyl, —O—C₁-C₆ alkyl, or C₆-C₁₂ aryl optionally substituted with one or more independently selected halo substituents;
   each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl;
   each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl;
   each R^{j}, R^{k}, R^{m}, Rⁿ, R^{o}, R^{p}, R^{g}, and R^{r} is independently hydrogen or C₁-C₆ alkyl;
   m is 0 or 1; and
   n is 0, 1, or 2;
R⁵ is hydrogen or is taken together with R^{b} and the intervening atoms form a 5- to 6-membered heterocycloalkyl or heterocycloalkenyl ring;
Z¹ and Z⁵ are each independently R^{z};
Z² and Z³ are each independently hydrogen or R^{z};
Z⁴ is hydrogen or R^{z} or is taken together with R² and the intervening atoms to form a 4-6 membered heterocycloalkyl or heterocycloalkenyl ring; and
R^{z} is selected from the group consisting of:
   a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy;
   b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
   c) C₁-C₆ alkoxy;
   d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R^{w} is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
   e) C₆-C₁₂ aryl; and
   f) 5- to 10-membered heteroaryl optionally substituted with one or more independently selected C₁-C₆ alkyl substituents,

wherein (1) when R⁴ is Z¹NR^{a}C(O)—, Z¹ is other than methyl, unsubstituted cyclopropyl, —C(CH₃)₂CH₂OH, and —CH₂-thiofuran;

(2) R⁴ is other than 4-methylpiperazinyl, 4-phenylpiperazinyl, 4-pyridylpiperazinyl, 4-(furanylmethyl)piperazinyl,

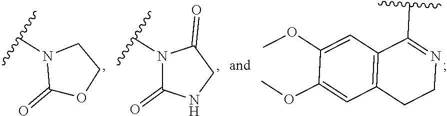

and

(3) the compound of Formula (I) is not a compound of Table 1X.

2. The compound of embodiment 1, or a pharmaceutically acceptable salt thereof, wherein R¹ is halo.
3. The compound of embodiment 1 or embodiment 2, or a pharmaceutically acceptable salt thereof, wherein R¹ is Cl.
4. The compound of embodiment 1, or a pharmaceutically acceptable salt thereof, wherein R¹ is methoxy.
5. The compound of any one of embodiments 1-4, or a pharmaceutically acceptable salt thereof, R² is hydrogen.
6. The compound of any one of embodiments 1-4, or a pharmaceutically acceptable salt thereof, R² is C₁-C₆ alkyl.
7. The compound of any one of embodiments 1-6, or a pharmaceutically acceptable salt thereof, R³ is hydrogen.
8. The compound of any one of embodiments 1-6, or a pharmaceutically acceptable salt thereof, R³ is C₁-C₆ alkyl.
9. The compound of any one of embodiments 1-6, or a pharmaceutically acceptable salt thereof, wherein the compound of Formula (I) is a compound of Formula (I-A)

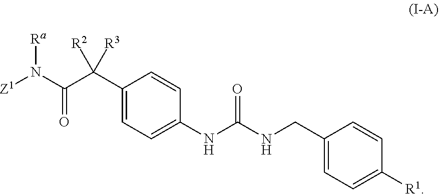

10. The compound of any one of embodiments 1-9, or a pharmaceutically acceptable salt thereof, wherein R^{a} is hydrogen.
11. The compound of any one of embodiments 1-9, or a pharmaceutically acceptable salt thereof, wherein R^{a} is C₁-C₆ alkyl.
12. The compound of any one of embodiments 1-11, or a pharmaceutically acceptable salt thereof, wherein Z¹ is selected from the group consisting of.

C₁-C₆ alkyl optionally substituted with one or more substituents independently selected from the group consisting of —OH, C₃-C₆ cycloalkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of C₁-C₆ alkyl and C₁-C₆ alkoxy;

C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with C₁-C₆ alkyl; and

3- to 10-membered heterocycloalkyl or heterocycloalkenyl optionally substituted with one or more substituents independently selected from the group consisting of —C₁-C₆ alkyl and —C(O)OC₁-C₆ alkyl, wherein the —C₁-C₆ alkyl is optionally substituted with C₆-C₁₂ aryl.

13. The compound of any one of embodiments 1-11, or a pharmaceutically acceptable salt thereof, wherein Z¹ is selected from the group consisting of ethyl,

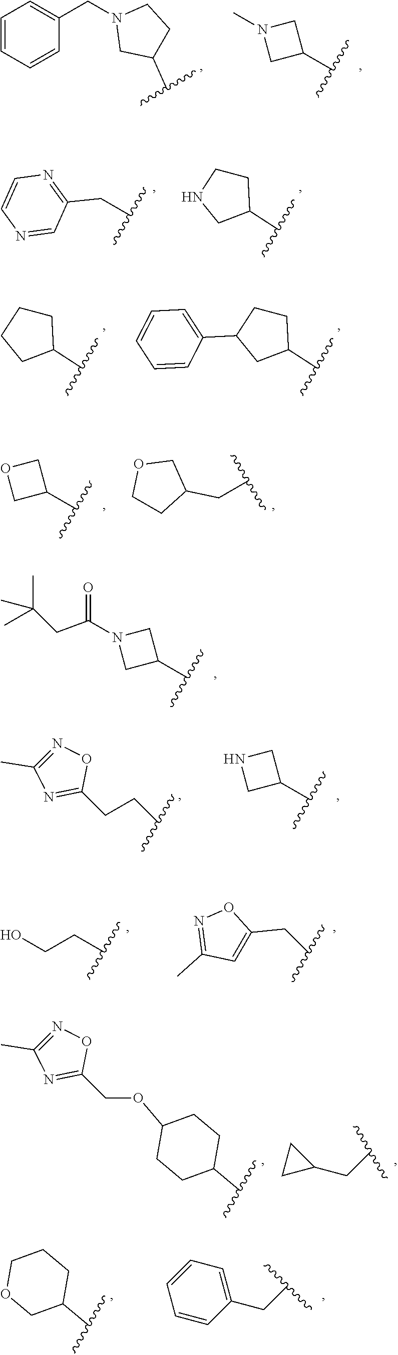

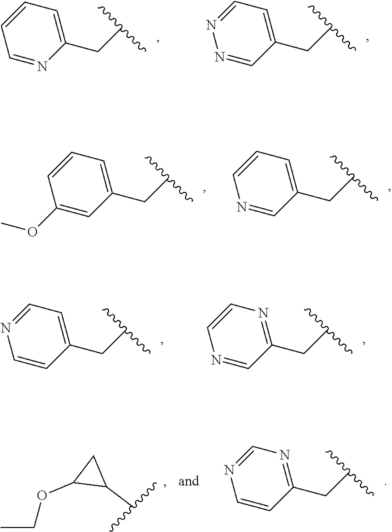

14. The compound of embodiment 1, or a pharmaceutically acceptable salt thereof, wherein the compound of Formula (I) is a compound of Formula (I-B)

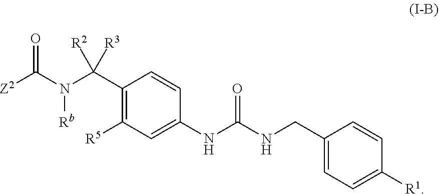

15. The compound of any one of embodiments 1-8 and 14, or a pharmaceutically acceptable salt thereof, wherein R^{b} is hydrogen.
16. The compound of any one of embodiments 1-8 and 14, or a pharmaceutically acceptable salt thereof, wherein R^{b} is C₁-C₆ alkyl.
17. The compound of any one of embodiments 1-8 and 14, or a pharmaceutically acceptable salt thereof, wherein R^{b} is taken together with R⁵ and the intervening atoms to form a 5- to 6-membered heterocycloalkyl or heterocycloalkenyl ring.
18. The compound of any one of embodiments 1-8 and 14-17, or a pharmaceutically acceptable salt thereof, wherein Z² is hydrogen.
19. The compound of any one of embodiments 1-8 and 14-17, or a pharmaceutically acceptable salt thereof, wherein Z² is selected from the group consisting of
C₁-C₆ alkyl optionally substituted with one or more substituents independently selected from the group consisting of C₃-C₆ cycloalkyl and 5- to 10-membered heteroaryl;
C₃-C₆ cycloalkyl optionally substituted with one or more substituents independently selected from the group consisting of C₁-C₆ alkyl and C₁-C₆ alkoxy;
C₁-C₆ alkoxy;
3- to 10-membered heterocycloalkyl or heterocycloalkenyl optionally substituted with one or more —C₁-C₆ alkyl substituents;
C₆-C₁₂ aryl; and
5- to 10-membered heteroaryl optionally substituted with one or more independently selected C₁-C₆ alkyl substituents.
   20. The compound of embodiment 19, or a pharmaceutically acceptable salt thereof, wherein Z² is a 5- to 6-membered heteroaryl optionally substituted with one or more —C₁-C₆ alkyl substituents.
   21. The compound of embodiment 20, or a pharmaceutically acceptable salt thereof, wherein Z² is a pyridyl group optionally substituted with one or more —C₁-C₆ alkyl substituents.
   22. The compound of any one of embodiments 1-8 and 14-17, or a pharmaceutically acceptable salt thereof, wherein Z² is selected from the group consisting of ethyl,

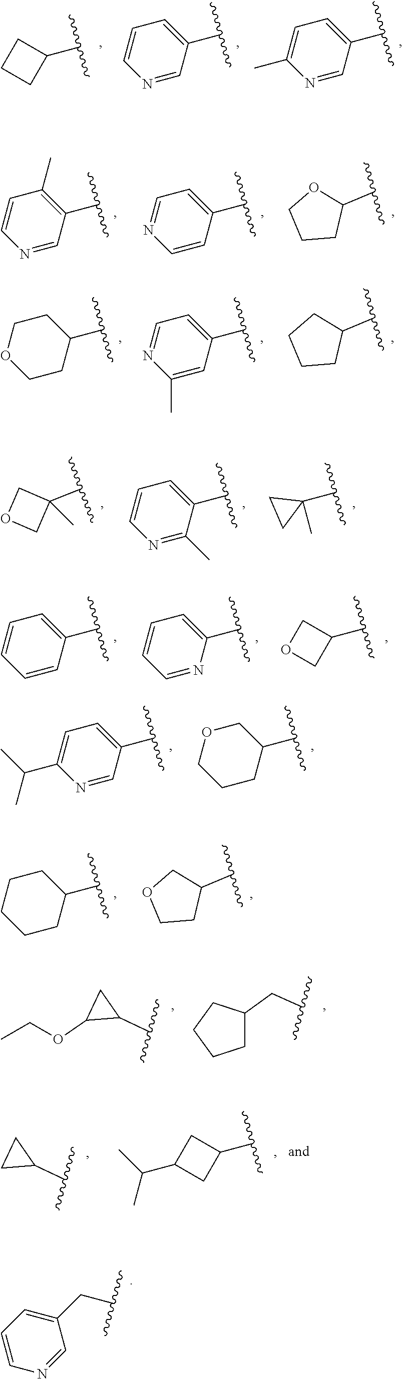

23. The compound of embodiment 22, or a pharmaceutically acceptable salt thereof, wherein Z² is

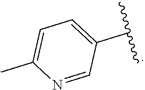 [SMILES: *c1ccc(C)nc1]

24. The compound of embodiment 1, or a pharmaceutically acceptable salt thereof, wherein the compound of Formula (I) is a compound of Formula (I-C)

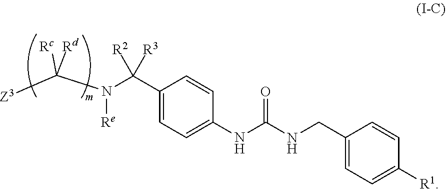

25. The compound of any one of embodiments 1-8 and 24, or a pharmaceutically acceptable salt thereof, wherein m is 1.
26. The compound of any one of embodiments 1-8 and 24, or a pharmaceutically acceptable salt thereof, wherein m is 0.
27. The compound of any one of embodiments 1-8, and 24-25, or a pharmaceutically acceptable salt thereof, wherein R^{c} is hydrogen.
28. The compound of any one of embodiments 1-8 and 24-25, or a pharmaceutically acceptable salt thereof, wherein R^{c} is C₁-C₆ alkyl.
29. The compound of any one of embodiments 1-8, 24-25, and 27-28, or a pharmaceutically acceptable salt thereof, wherein R^{d} is hydrogen.
30. The compound of any one of embodiments 1-8, 24-25, and 27-28, or a pharmaceutically acceptable salt thereof, wherein R^{d} is C₁-C₆ alkyl.
31. The compound of any one of embodiments 1-8 and 24-25, or a pharmaceutically acceptable salt thereof, wherein R^{c} and R^{d} together with the carbon to which they are attached form a C₃-C₆ cycloalkyl.
32. The compound of any one of embodiments 1-8 and 24-31, or a pharmaceutically acceptable salt thereof, wherein Reis hydrogen.
33. The compound of any one of embodiments 1-8 and 24-31, or a pharmaceutically acceptable salt thereof, wherein R^{e} is C₁-C₆ alkyl.
34. The compound of any one of embodiments 1-8 and 24-33, or a pharmaceutically acceptable salt thereof, wherein Z³ is hydrogen.
35. The compound of any one of embodiments 1-8 and 24-33, or a pharmaceutically acceptable salt thereof, wherein Z³ is selected from the group consisting of
C₃-C₆ cycloalkyl;
3- to 10-membered heterocycloalkyl or heterocycloalkenyl optionally substituted with —C₁-C₆ alkyl;
C₆-C₂ aryl; and
5- to 10-membered heteroaryl optionally substituted with one or more independently selected C₁-C₆ alkyl substituents.
   36. The compound of any one of embodiments 1-8 and 24-33, or a pharmaceutically acceptable salt thereof, wherein Z³ is selected from the group consisting of

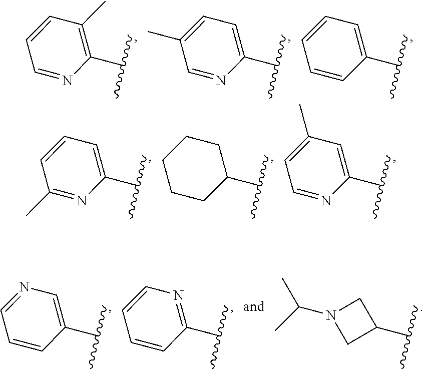

37. The compound of embodiment 1, or a pharmaceutically acceptable salt thereof, wherein the compound of Formula (I) is a compound of Formula (I-D)

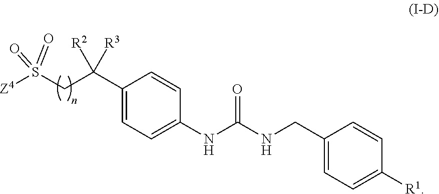

38. The compound of any one of embodiments 1-8 and 37, or a pharmaceutically acceptable salt thereof, wherein n is 0.
39. The compound of any one of embodiments 1-8 and 37, or a pharmaceutically acceptable salt thereof, wherein n is 1.
40. The compound of any one of embodiments 1-8 and 37, or a pharmaceutically acceptable salt thereof, wherein n is 2.
41. The compound of any one of embodiments 1-8 and 37-40, or a pharmaceutically acceptable salt thereof, wherein Z⁴ is hydrogen or R^{z}.
42. The compound of any one of embodiments 1-8 and 37-40 or a pharmaceutically acceptable salt thereof, wherein Z⁴ is C₁-C₆ alkyl.
43. The compound of any one of embodiments 1-8 and 37-40, or a pharmaceutically acceptable salt thereof, wherein Z⁴ is taken together with R² and the intervening atoms to form a 4-6 membered heterocycloalkyl or heterocycloalkenyl ring.
44. The compound of embodiment 43, wherein

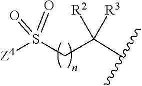

is selected from the group consisting of

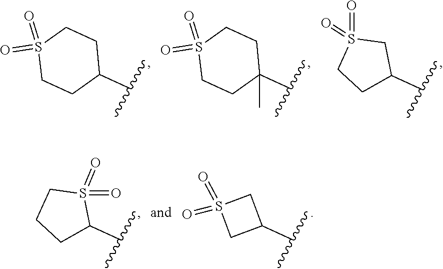

45. The compound of embodiment 1, or a pharmaceutically acceptable salt thereof, wherein the compound of Formula (I) is a compound of Formula (I-E)

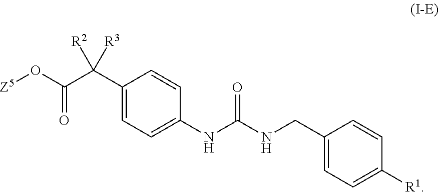

46. The compound of any one of embodiments 1-8 and 45, or a pharmaceutically acceptable salt thereof, wherein Z⁵ is C₁-C₆ alkyl.
47. The compound of any one of embodiments 1-8 and 45, or a pharmaceutically acceptable salt thereof, wherein Z⁵ is ethyl.
48. The compound of claim 1, or a pharmaceutically acceptable salt thereof, wherein the compound of Formula (I) is a compound of Formula (I-F)

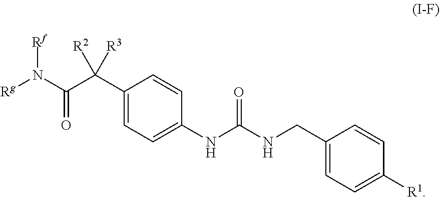

49. The compound of any one of claims 1-8 and 48, or a pharmaceutically acceptable salt thereof, wherein R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl.
50. The compound of claim 49, or a pharmaceutically acceptable salt thereof, wherein R^{f} and R^{g} together with the nitrogen to which they are attached form a 5- to 6-membered heterocycloalkyl or heterocycloalkenyl optionally substituted with —C₁-C₆ alkyl, wherein the —C₁-C₆ alkyl is optionally substituted with —OH.
51. The compound of any one of embodiments 1-8 and 48-49, or a pharmaceutically acceptable salt thereof, wherein

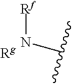

is selected from the group consisting of

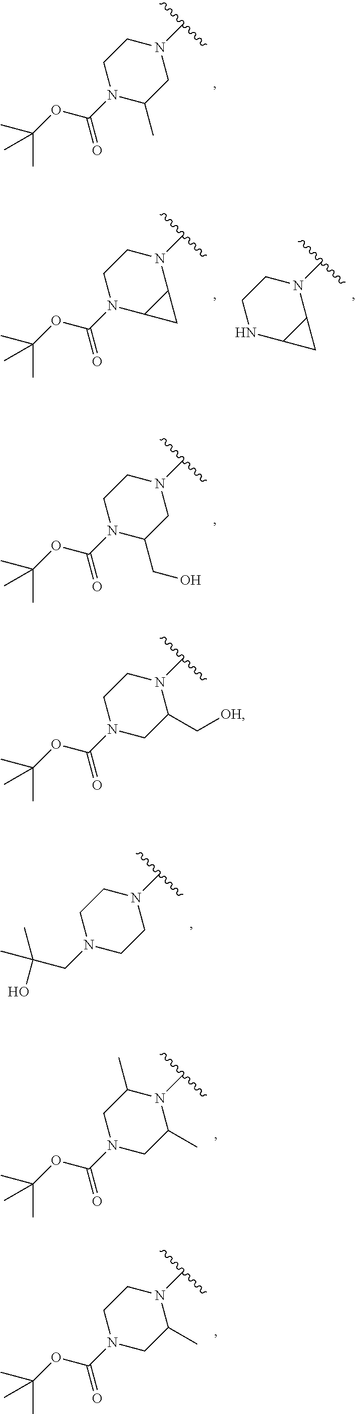

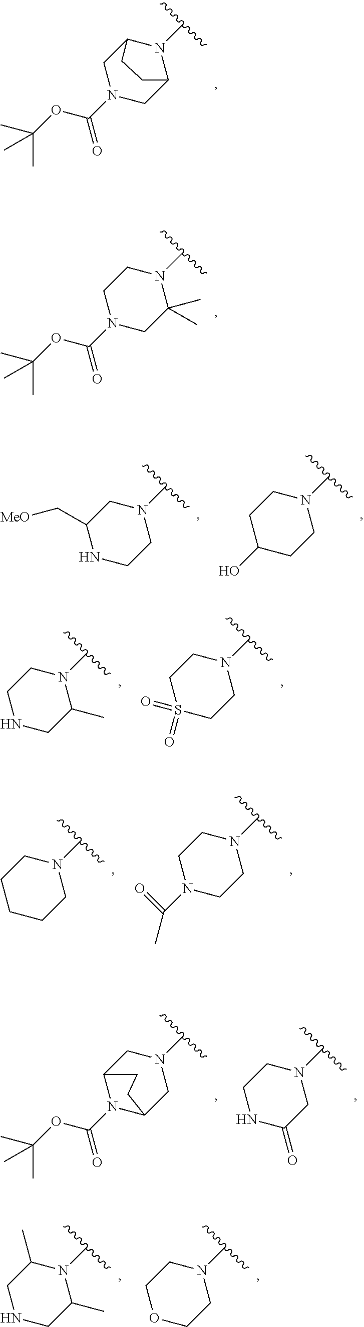

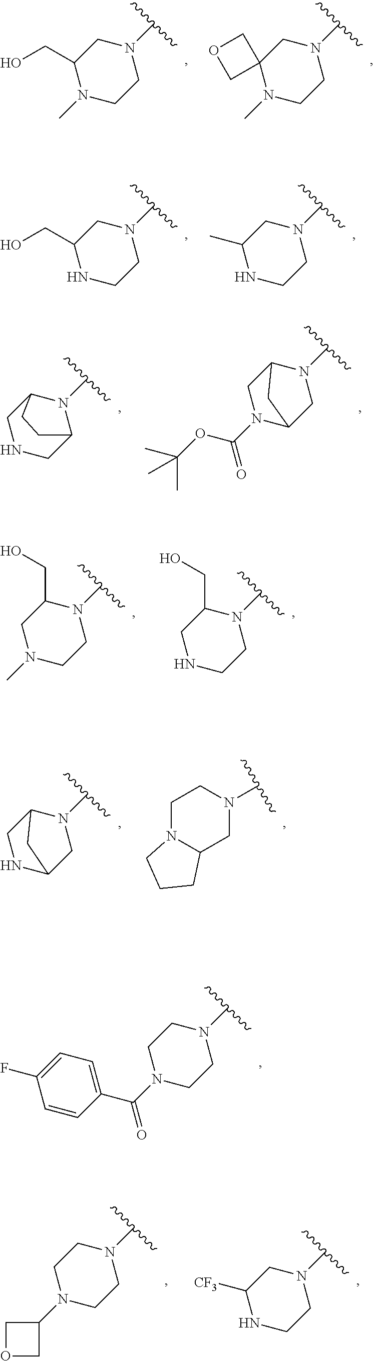

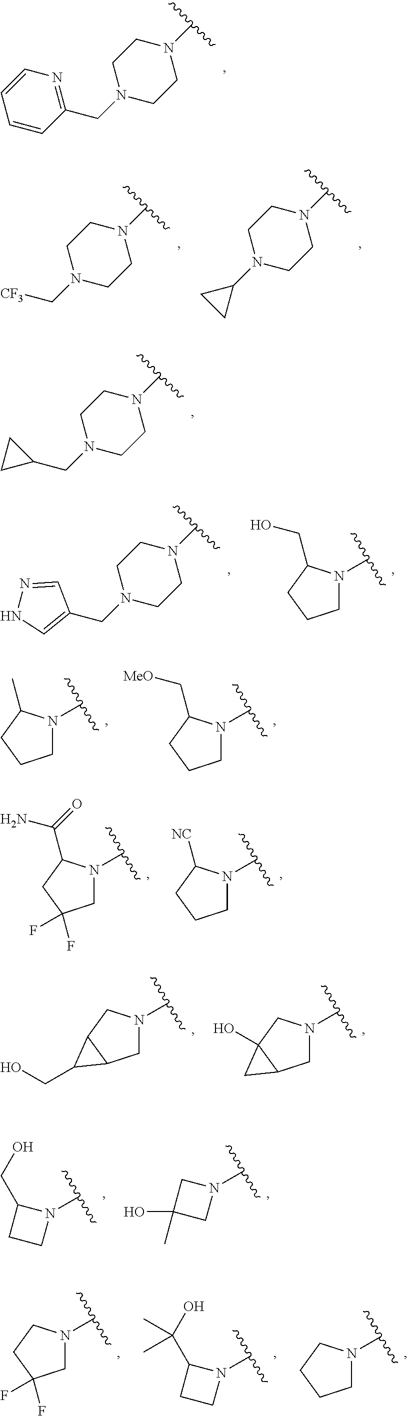

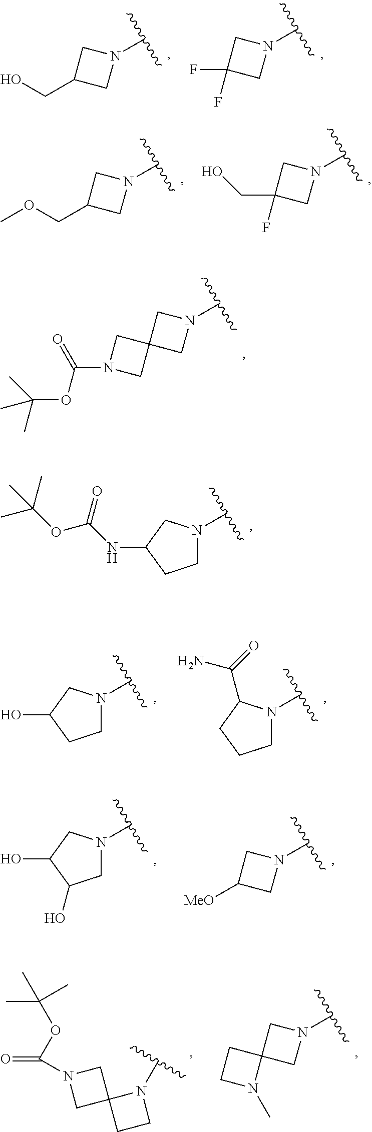

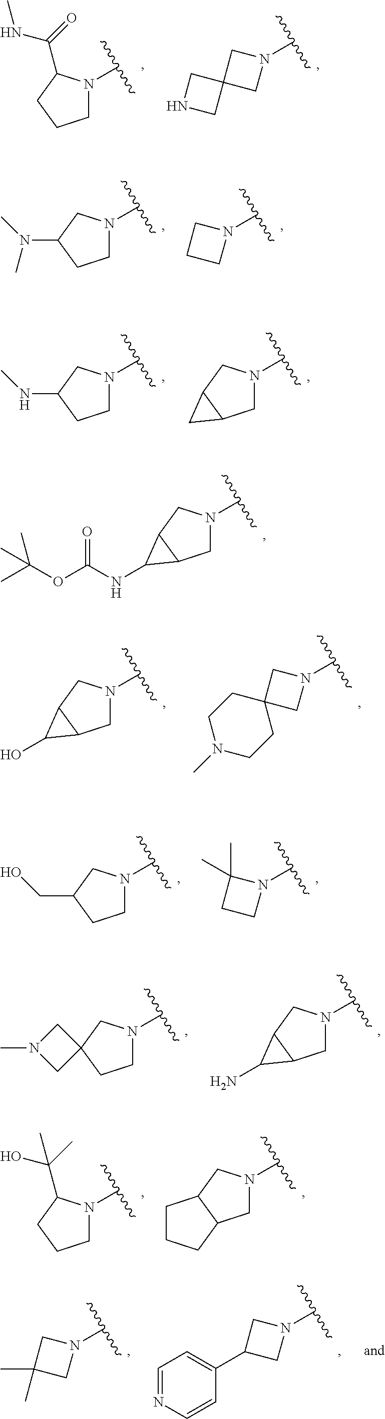

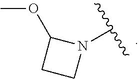 [SMILES: *N1CCC1OC]

52. The compound of embodiment 51, or a pharmaceutically acceptable salt thereof, wherein

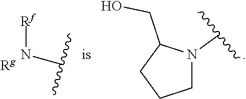

53. The compound of any one of embodiments 1-8, or a pharmaceutically acceptable salt thereof, wherein R⁴ is a 5- to 10 membered heteroaryl optionally substituted with one or more independently selected C₁-C₆ alkyl substituents.
54. The compound of any one of embodiments 1-8 and 53, or a pharmaceutically acceptable salt thereof, wherein R⁴ is selected from the group consisting of

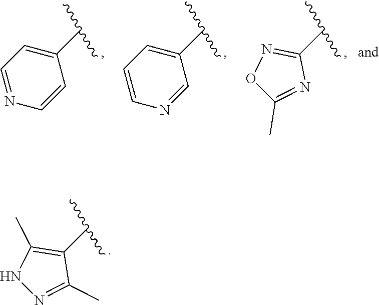

55. The compound of any one of embodiments 1-8, or a pharmaceutically acceptable salt thereof, wherein R⁴ is a 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents.
56. The compound of embodiment 55, or a pharmaceutically acceptable salt thereof, wherein R⁴ is a 4- to 6-membered heterocycloalkyl or heterocycloalkenyl optionally substituted with —S(O)₂—C₁-C₆ alkyl or —C₁-C₆ alkyl optionally substituted with —OH.
57. The compound of any one of embodiments 1-8 and 55, or a pharmaceutically acceptable salt thereof, wherein R⁴ is selected from the group consisting of

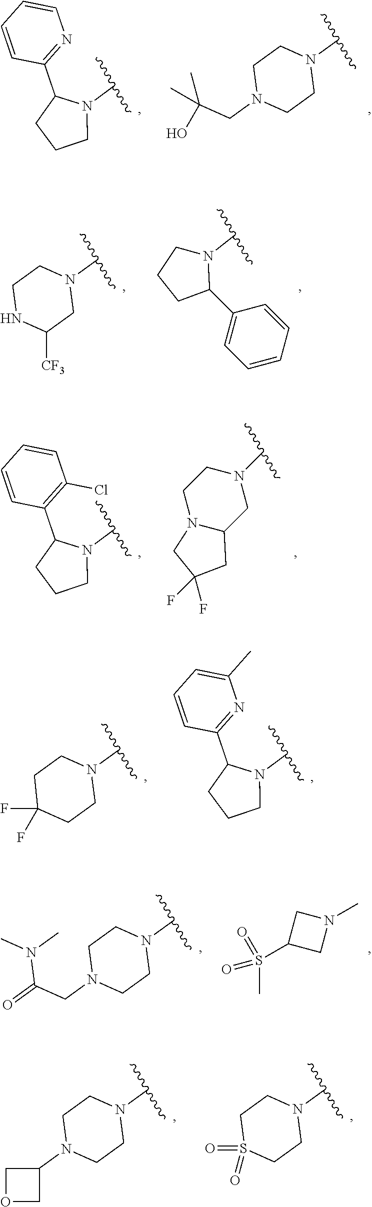

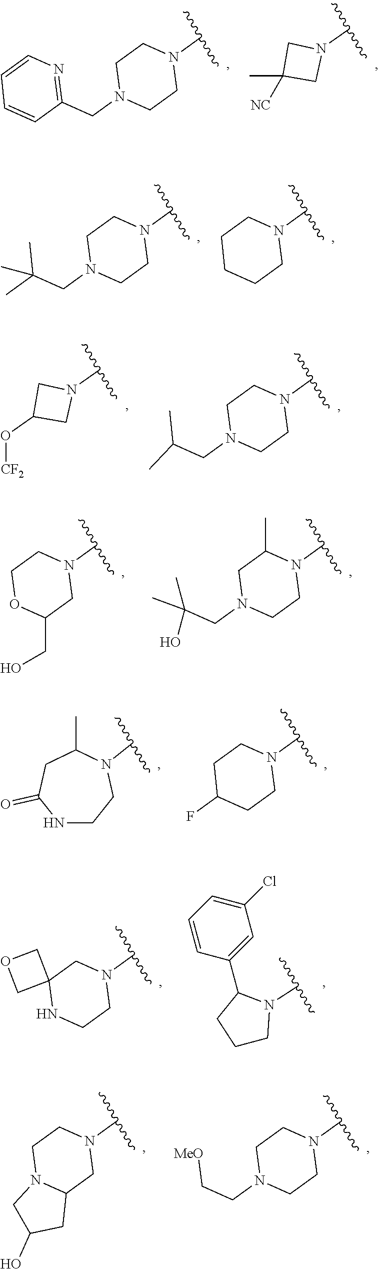

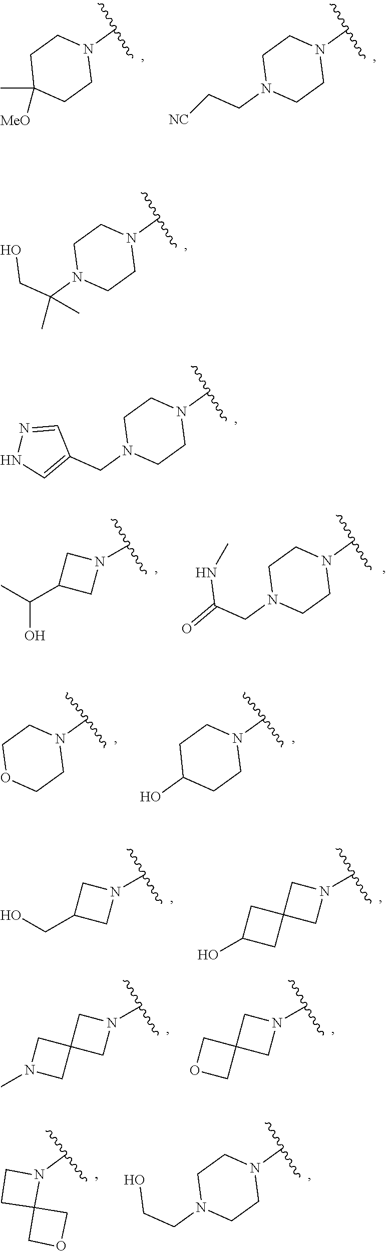

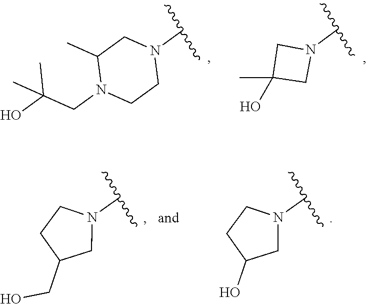

58. The compound of embodiment 57, or a pharmaceutically acceptable salt thereof, wherein R⁴ is

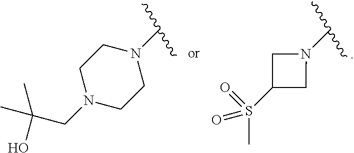

59. A compound selected from the group consisting of compounds of Table 1, or a pharmaceutically acceptable salt thereof.
60. A pharmaceutical composition comprising a compound according to any one of embodiments 1-59, or a pharmaceutically acceptable salt thereof, and a pharmaceutically acceptable excipient.
61. A method of treating a disease or condition mediated by NAMPT activity in a subject in need thereof, comprising administering to the subject a compound of any one of embodiments 1-59, or a pharmaceutically acceptable salt thereof, or a pharmaceutical composition of embodiment 60.
62. The method of embodiment 61, wherein the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder.
63. The method of embodiment 61, wherein the disease or condition is selected from the group consisting of obesity, atherosclerosis, insulin resistance, type 2 diabetes, cardiovascular disease, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.

### General Synthetic Methods

Compounds of Formula (II), (I-G), (I) (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D32), (I-D33), (I-D4), (I-D5), (I-D6), (I-D7), (I-E), (I-F), (II-A), and (II-A1) will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate. Unless otherwise specified, the variables are as defined above in reference to Formula (II), (I-G), (I) (I-A), (I-A1), (I-A2), (I-A3), (I-A4), (I-B), (I-B31), (I-B32), (I-B33), (I-C), (I-C1), (I-C2), (I-C3), (I-C4), (I-D), (I-D1), (I-D2), (I-D3), (I-D4), (I-D5), (I-D6), (I-D7), (I-E), (I-F), (II-A), and (II-A1).

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g. a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

General methods of preparing compounds described herein are depicted in exemplified methods below. Variable groups in the schemes provided herein are defined as for Formula (II), (I-G), (I) (I-A), (I-A1), (I-A2), (I-A3), (I-A4), (I-B), (I-B1), (I-B2), (I-B3), (I-C), (I-C1), (I-C2), (I-C3), (I-C4), (I-D), (I-D1), (I-D32), (I-D33), (I-D34), (I-D35), (I-D36), (I-D7), (I-E), (I-F), (II-A), and (II-A1), or any variation thereof. Other compounds described herein may be prepared by similar methods.

In some embodiments, compounds provided herein may be synthesized according to Scheme A1, A2, or A3.

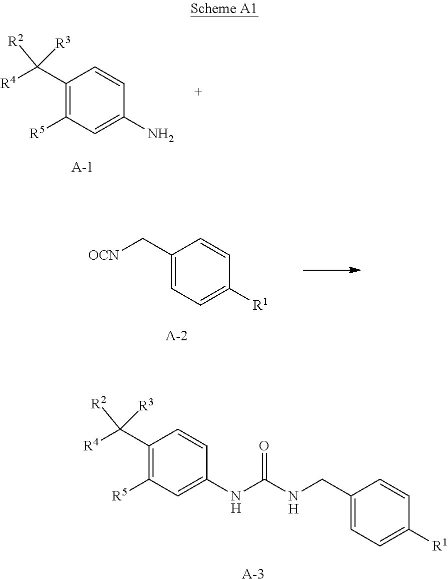

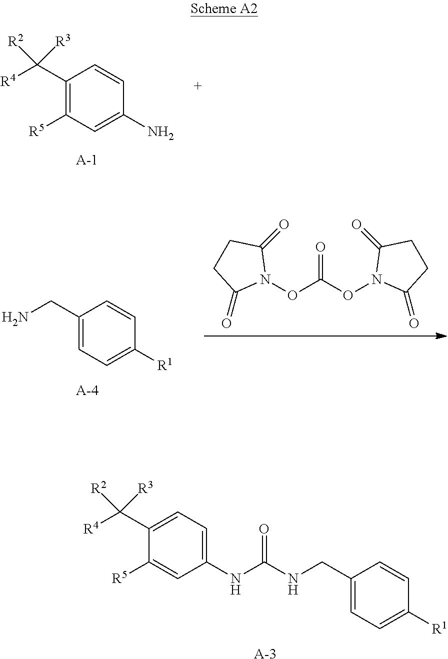

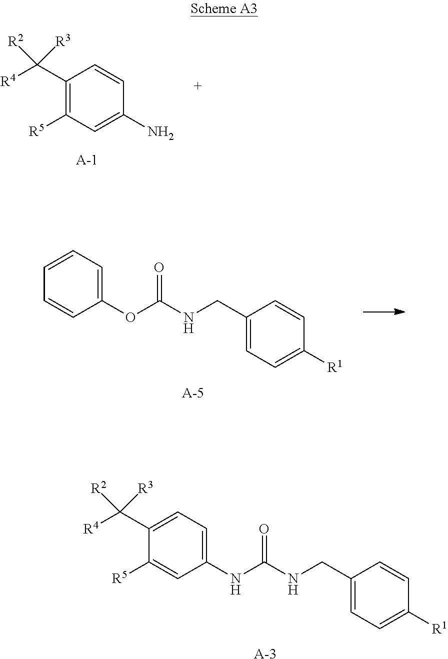

wherein R¹, R², R³, R⁴, and R⁵ are as defined for formula (II) or any variation thereof detailed herein.

In certain embodiments compounds provided herein may be synthesized according to Scheme A1a, A2a, or A3a:

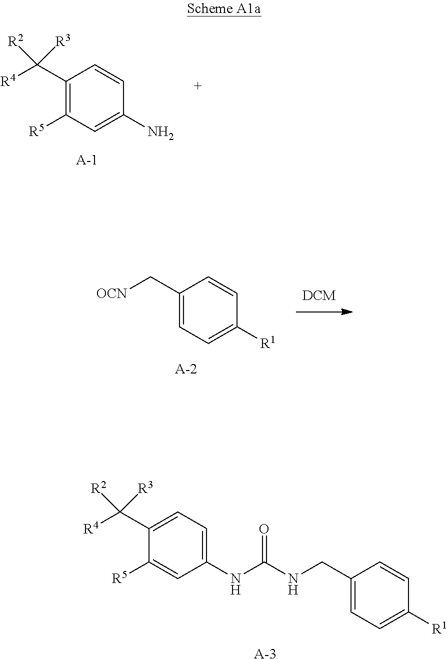

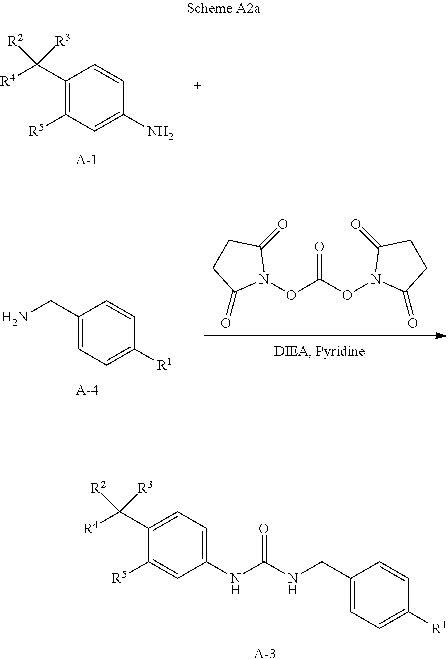

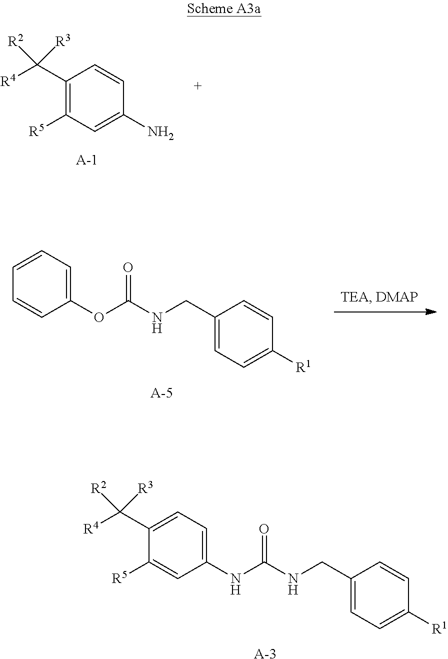

wherein R¹, R², R³, R⁴, and R⁵ are as defined for formula (II) or any variation thereof detailed herein.

In some embodiments, compounds provided herein may be synthesized according to Scheme B1 or B2:

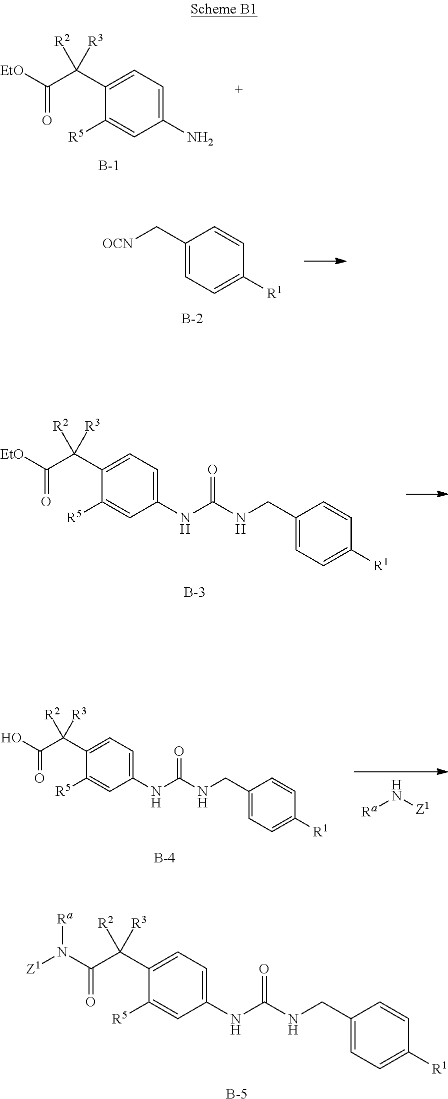

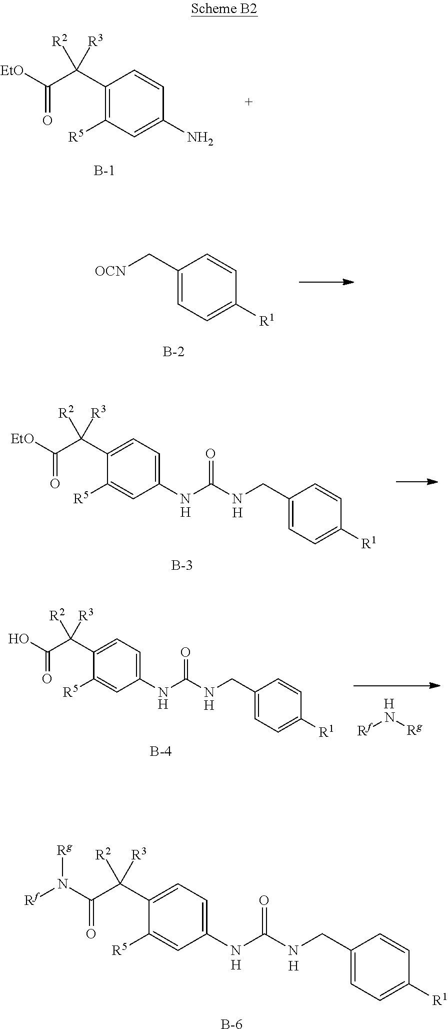

wherein R¹, R², R³, R⁵, R^{a}, R^{g}, R^{f}, and Z¹ are as defined for formula (II) or any variation thereof detailed herein.

In certain embodiments compounds provided herein may be synthesized according to Scheme B1a or B2a:

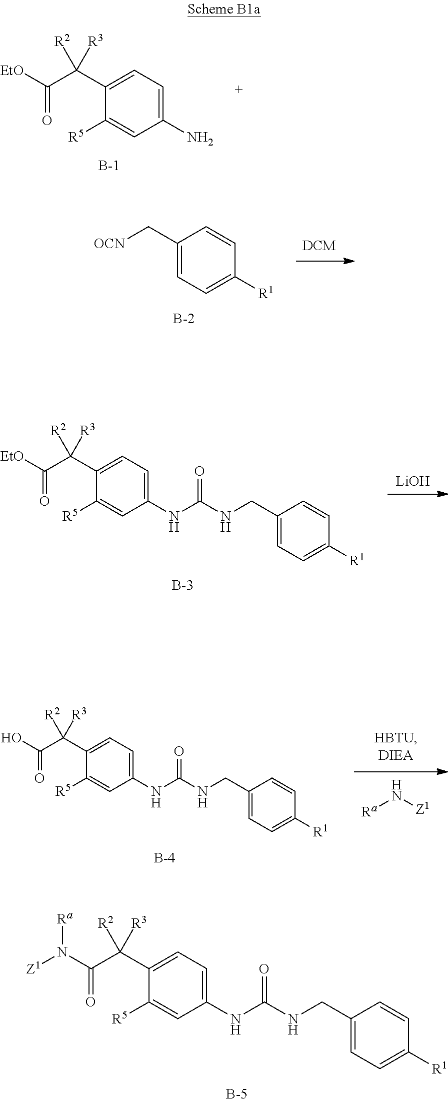

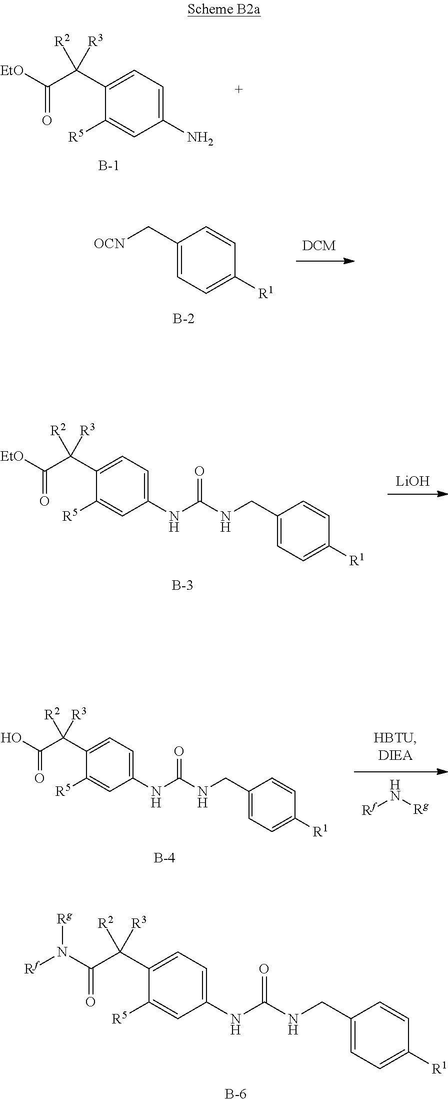

wherein R¹, R², R³, R⁵, R^{a}, R^{g}, R^{f}, and Z¹ are as defined for formula (II) or any variation thereof detailed herein.

In some embodiments, compounds provided herein may be synthesized according to Scheme C1 or C2:

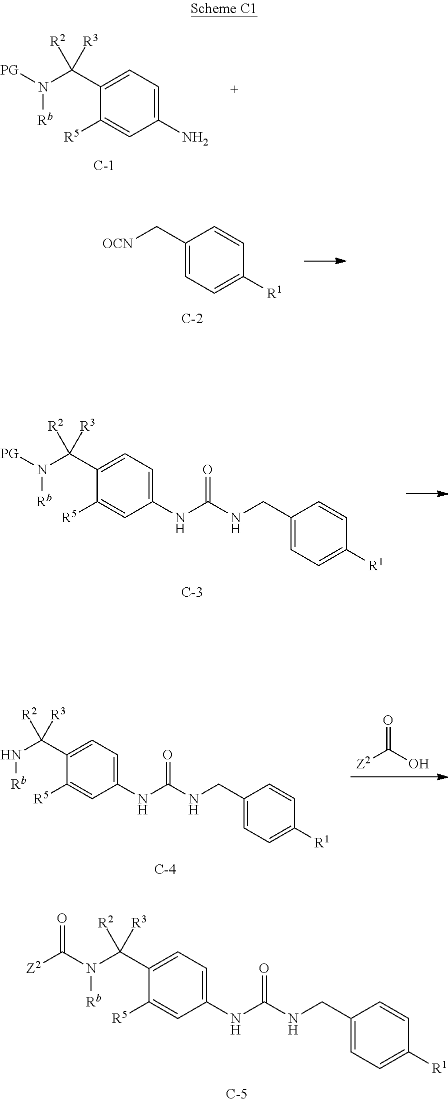

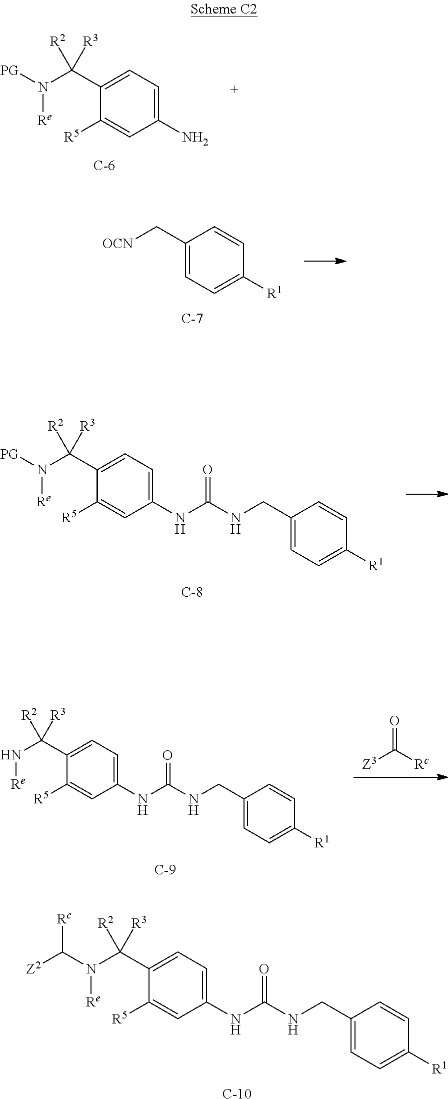

wherein R¹, R², R³, R⁵, R^{b}, R^{c}, R^{e}, Z² and Z³ are as defined for formula (II) or any variation thereof detailed herein, and PG is a suitable protecting group.

In certain embodiments, compounds provided herein may be synthesized according to Scheme C1a or C2a:

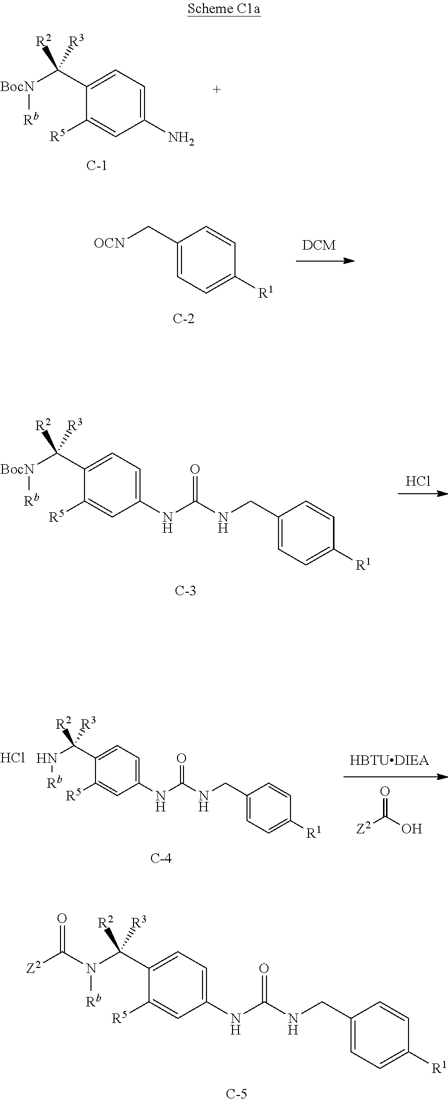

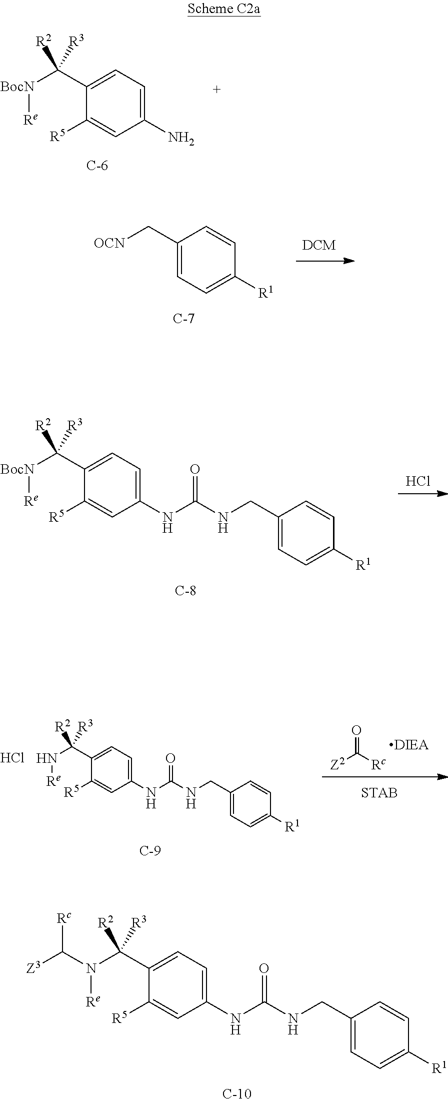

wherein R¹, R², R³, R⁵, R^{b}, R^{c}, R^{e}, Z² and Z³ are as defined for formula (II) or any variation thereof detailed herein.

In some embodiments, compounds provided herein may be synthesized according to Scheme D1:

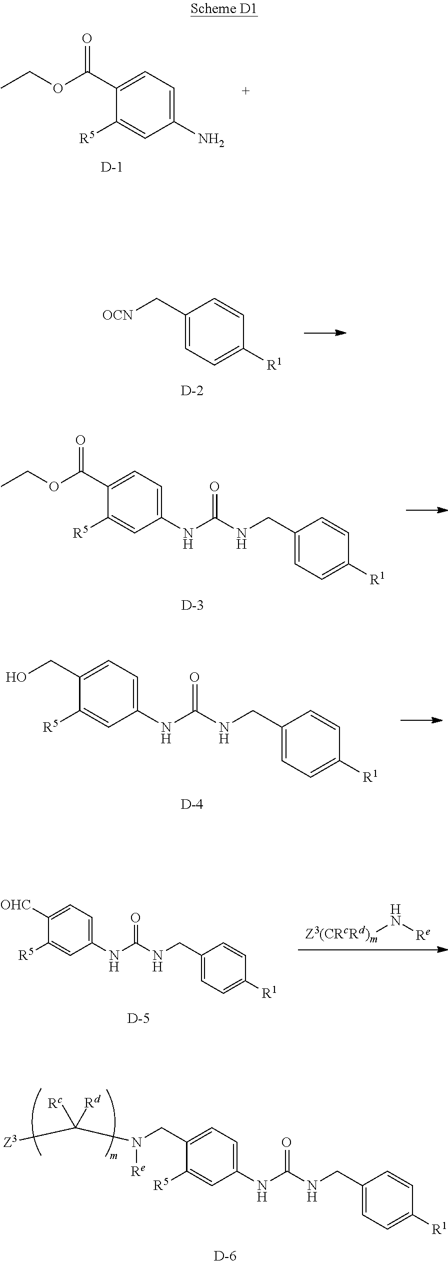

wherein R¹, R⁵, R^{c}, R^{d}, m, and Z³ are as defined for formula (II) or any variation thereof detailed herein, and PG is a suitable protecting group.

In certain embodiments, compounds provided herein may be synthesized according to Scheme D1a:

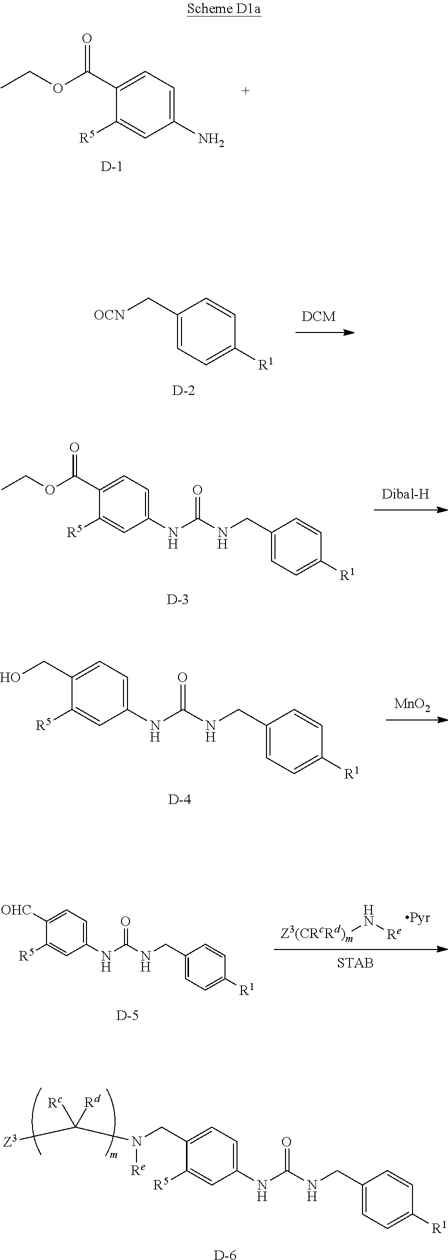

wherein R¹, R⁵, R^{c}, R^{d}, m, and Z³ are as defined for formula (II) or any variation thereof detailed herein.

In some embodiments, compounds provided herein may be synthesized according to Scheme E1:

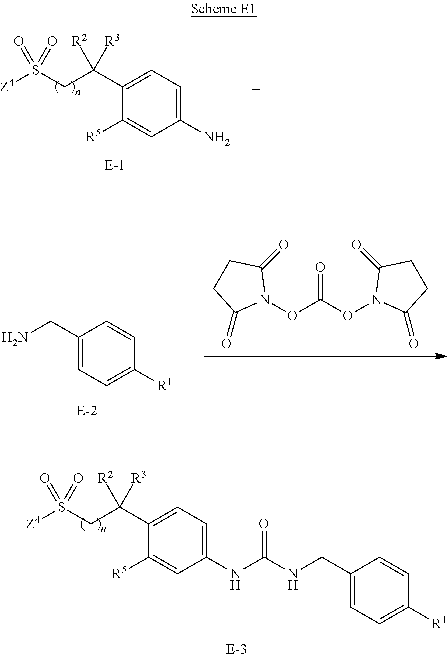

wherein R¹, R², R³, R⁵, n, and Z⁴ are as defined for formula (II) or any variation thereof detailed herein.

In certain embodiments, compounds provided herein may be synthesized according to Scheme E1a:

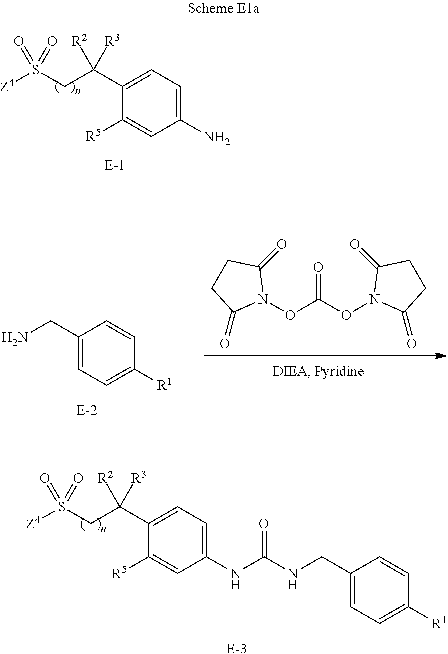

wherein R¹, R², R³, R⁵, n, and Z⁴ are as defined for formula (II) or any variation thereof detailed herein.

In some embodiments, compounds provided herein may be synthesized according to Schemes F1:

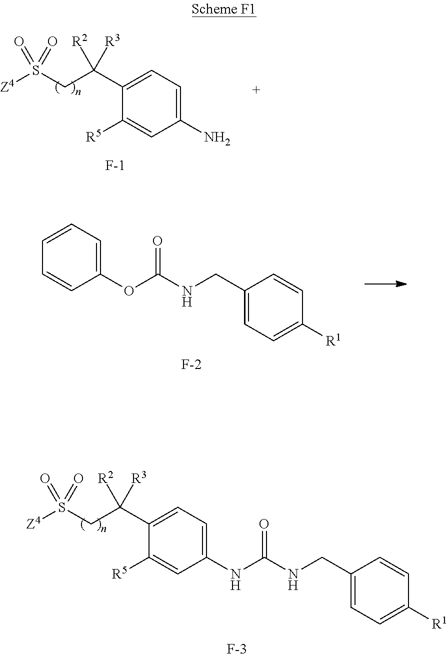

wherein R¹, R², R³, R⁵, n, and Z⁴ are as defined for formula (II) or any variation thereof detailed herein.

In certain embodiments, compounds provided herein may be synthesized according to Schemes F1a:

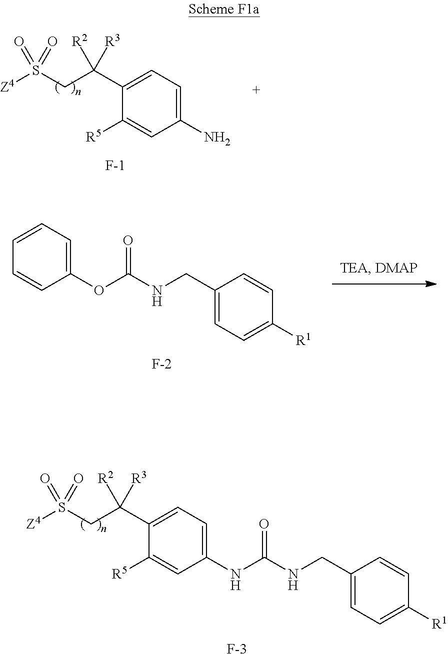

wherein R¹, R², R³, R⁵, n, and Z⁴ are as defined for formula (II) or any variation thereof detailed herein.

Particular non-limiting examples are provided in the Example section below.

### EXAMPLES

The following examples are offered to illustrate but not to limit the compositions, uses, and methods provided herein. The compounds are prepared using the general methods described above.

The following abbreviations are used throughout the Examples: TEA (triethylamine), DCM (dichloromethane), (Boc)₂O (di-tert-butyl decarbonate), EA (Ethyl acetate), PE (Petroleum ether, DMF (N,N-dimethylformamide), DIEA (N-ethyl-N-isopropylpropan-2-amine), HATU (1-[Bis(dimethylamino)methylene]-1H-1,2,3-triazolo[4,5-b]pyridinium 3-oxid hexafluorophosphate), HOAt (1-Hydroxy-7-azabenzotriazole), HOBt (Hydroxybenzotriazole), EDCI (1-Ethyl-3-(3-dimethylaminopropyl)carbodiimide), MeOH (methanol), EtOH (ethanol), iPrOH (propan-2-ol), ACN (acetonitrile), TFA (trifluoroacetic acid), DPPA (Diphenylphosphoryl azide), DBU (1,8-Diazabicyclo(5.4.0)undec-7-ene), THE (tetrahydrofuran), PPh₃ (triphenylphosphane), SM (starting material), Hex (hexane), NCS (N-chlorosuccinimide), r.t. (room temperature), DCE (dichloroethane), FA (formic acid), CHCl₃ (Chloroform), BnBr (benzyl bromide), HCl (hydrogen chloride), equiv (equivalent), and DSC (bis(2,5-dioxopyrrolidin-1-yl) carbonate), HBTU (0-(benzotriazol-1-yl)-N,N,N′,N′-tetramethyluronium hexafluorophosphate).

### Example A

### Synthesis of Intermediates 1.1, 1.2, 1.3 and 1.4

### Step 1: Preparation of 2-(4-(3-(4-methoxybenzyl)ureido)phenyl)acetic acid (Intermediate 1-a)

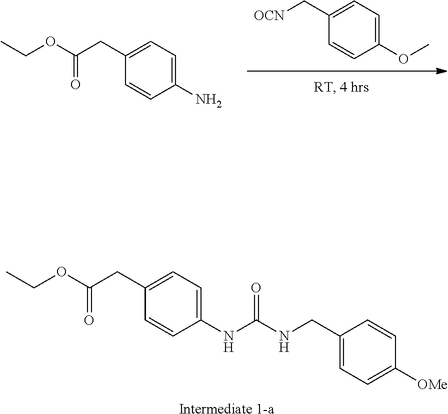

To a solution of ethyl 2-(4-aminophenyl)acetate (27.46 g, 153.2 mmol) in DCM (20 mL) at 20° C. was added 4-methoxy benzyl isocyanate (25.0 g, 153.2 mmol) dropwise. The resulting mixture was stirred at room temperature for 4 hours then methanol (10 mL) was added and cooled to 0° C. After 1 hour at 0° C. the slurry was filtered providing Intermediate 1-a (26.7 g, 78.0 mmol, 50.9% yield) as an off-white solid. LCMS-APCI (POS.) m/z: 343.1 (M+H)⁺. 1H NMR (400 MHz, DMSO-d6) δ 8.50 (s, 1H), 7.38-7.30 (m, 2H), 7.27-7.19 (m, 2H), 7.15-7.07 (m, 2H), 6.94-6.85 (m, 2H), 6.52 (t, J=5.9 Hz, 1H), 4.22 (d, J=5.4 Hz, 2H), 4.06 (q, J=7.1 Hz, 2H), 3.73 (s, 3H), 3.55 (s, 2H), 1.17 (t, J=7.1 Hz, 3H).

### Step 2: Preparation of 2-(4-(3-(4-methoxybenzyl)ureido)phenyl)acetic acid (Intermediate 1.1)

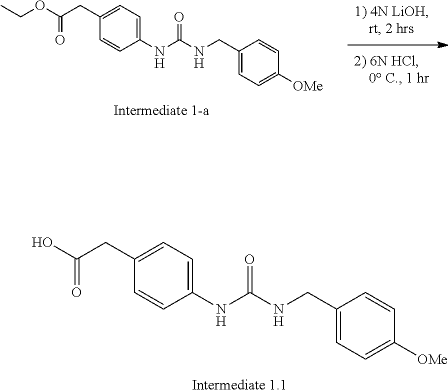 [SMILES: CCOC(=O)Cc1ccc(NC(=O)NCc2ccc(OC)cc2)cc1 ; "Intermediate 1-a" ; COc1ccc(CNC(=O)Nc2ccc(CC(=O)O)cc2)cc1 ; "Intermediate 1.1"]

To a solution of Intermediate 1-a (26.5 g, 77.5 mmol) in 1,4 dioxane (400 mL) at 20° C. was added 4 N LiOH (234.0 mmol) dropwise. The resulting mixture was stirred at room temperature for 2 hours then methanol (50 mL) was added. The pH of mixture was adjusted to pH 1-2 using aqueous 6N HCl at 0° C. After 1 hour at 0° C., the slurry was filtered providing 2-(4-(3-(4-methoxybenzyl)ureido)phenyl)acetic acid (20.2 g, 64.3 mmol, 82.9% yield) as an off-white solid. LCMS-APCI (POS.) m/z: 315.0 (M+H)⁺. ¹H NMR (400 MHz, DMSO-d₆) δ 12.22 (s, 1H), 8.47 (s, 1H), 7.33 (d, J=8.0 Hz, 2H), 7.23 (d, J=8.1 Hz, 2H), 7.11 (d, J=8.1 Hz, 2H), 6.90 (d, J=8.1 Hz, 2H), 6.50 (t, J=6.0 Hz, 1H), 4.22 (d, J=5.7 Hz, 2H), 3.74 (d, J=1.3 Hz, 3H), 3.46 (s, 2H).

Intermediates 1.2 and 1.3 were prepared in a similar manner as Intermediate 1.1, using the reagents provided in the table below in place of 4-methoxy benzyl isocyanate.

| Intermediate | Reagents | Structure, Name and Data |
|---|---|---|
| 1.2 | 4-chloro benzyl isocyanate | 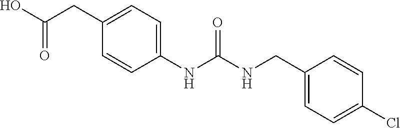 [SMILES: O=C(O)Cc1ccc(NC(=O)NCc2ccc(Cl)cc2)cc1] 2-(4-(3-(4-chlorobenzyl)ureido)phenyl)acetic acid. LCMS-ESI (POS.) m/z: 319.0 (M + H)+. ¹H NMR (400 MHz, DMSO-d6) δ 12.24 (s, 1H), 8.58 (s, 1H), 7.43-7.36 (m, 2H), 7.36-7.30 (m, 4H), 7.11 (d, J = 8.0 Hz, 2H), 6.66 (t, J = 6.0 Hz, 1H), 3.46 (s, 2H), 4.28 (d, J = 5.9 Hz, 2H). |
| 1.3 | 4-fluoro benzyl isocyanate | 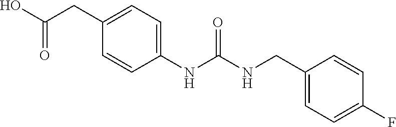 [SMILES: O=C(O)Cc1ccc(NC(=O)NCc2ccc(F)cc2)cc1] 2-(4-(3-(4-fluorobenzyl)ureido)phenyl)acetic acid. LCMS-ESI (POS.) m/z: 303.0 (M + H)+. ¹H NMR (400 MHz, DMSO-d₆) δ 12.45-12.04 (m, 1H), 8.54 (s, 1H), 7.34 (dd, J = 8.3, 5.6 Hz, 4H), 7.23-7.03 (m, 4H), 6.61 (t, J = 6.0 Hz, 1H), 4.28 (d, J = 5.9 Hz, 2H), 3.46 (s, 2H). |
| 1.4 | 4-methoxy benzyl isocyanate | 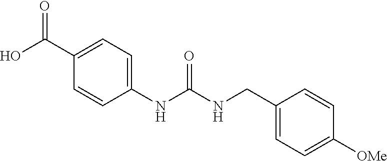 [SMILES: COc1ccc(CNC(=O)Nc2ccc(C(=O)O)cc2)cc1] 4-(3-(4-methoxybenzyl)ureido)benzoic acid. LCMS-APCI (POS.) m/z: 301.1 (M + H)⁺. 1H NMR (400 MHz, DMSO-d6) δ 9.72 (s, 1 H), 7.83 (t, J = 5.7 Hz, 1 H), 7.76 (d, J = 8.6 Hz, 2 H), 7.39 (d, J = 8.6 Hz, 2 H), 7.25 (d, J = 8.6 Hz, 2 H), 6.88 (d, J = 8.6 Hz, 2 H), 4.22 (d, J = 5.8 Hz, 2 H), 3.72 (s, 3 H). |

### Example B

### Synthesis of Intermediates 2.1, 2.2, 2.3, 2.4, 2.5, 2.6, and 2.7

### Step 1: Preparation of tert-butyl (S)-(1-(4-(3-(4-methoxybenzyl)ureido)phenyl)-ethyl)carbamate (Intermediate 2-a)

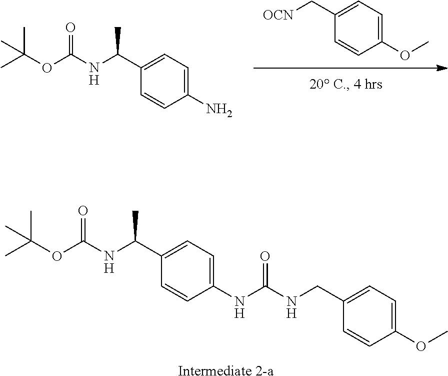

To a solution of (S)-[1-(4-amino-phenyl)-ethyl]-carbamic acid tert-butyl ester (2.0 g, 22.7 mmol) in DCM (20 mL) at 20 C was added 4-methoxy benzyl isocyanate (14.4 g, 34.0 mmol) dropwise. The resulting mixture was stirred at room temperature for 4 hours then methanol (10 mL) was added and cooled to 0° C. After 1 hour at 0° C. the slurry was filtered providing the tert-Butyl (S)-(1-(4-(3-(4-methoxybenzyl)ureido)-phenyl)ethyl)carbamate (1.2 g, 6.3 mmol, 28% yield) as an off-white solid. LCMS-APCI (POS.) m/z: 400.1 (M+H)⁺. ¹H NMR (400 MHz, DMSO-d₆) δ 8.43 (s, 1H), 7.36-7.19 (m, 4H), 7.14 (d, J=8.2 Hz, 2H), 6.89 (d, J=8.2 Hz, 2H), 6.48 (t, J=5.9 Hz, 1H), 4.53 (p, J=7.3 Hz, 1H), 4.21 (d, J=5.7 Hz, 2H), 3.73 (s, 3H), 1.37 (s, 9H), 1.27 (d, J=7.0 Hz, 3H).

### Step 2: Preparation of (S)-1-(4-(1-aminoethyl)phenyl)-3-(4-methoxybenzyl)urea hydrochloride (Intermediate 2.1)

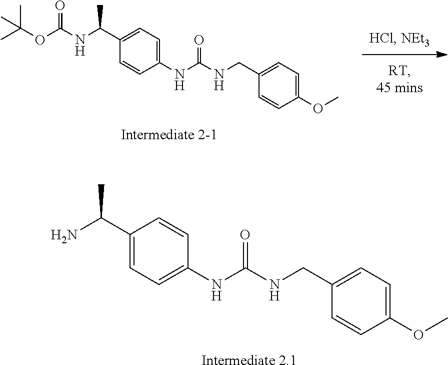 [SMILES: COc1ccc(CNC(=O)Nc2ccc([C@H](C)NC(=O)OC(C)(C)C)cc2)cc1 ; "Intermediate 2-1" ; COc1ccc(CNC(=O)Nc2ccc([C@H](C)N)cc2)cc1 ; "Intermediate 2.1"]

Intermediate 2-a (34.7 g, 86.9 mmol) was dissolved in dichloromethane and cooled to 0° C. with an ice bath. Hydrogen chloride (4 N in 1, 4-dioxane, 174 mL, 695 mmol) was added dropwise using a syringe, and the resulting mixture was stirred at 0° C. for 5 minutes before the ice bath was removed. The reaction was stirred at room temperature for 45 minutes and the reaction progress was monitored with LC/MS. It was quenched with triethylamine (28 mL) and the resulting mixture was concentrated in vacuo, providing a white solid. The solid was partitioned between saturated NaHCO₃ solution and DCM. The layers were separated and the aqueous phase was extracted with additional DCM. The organic extracts were combined, dried over Na₂SO₄ and concentrated under reduced pressure, providing (S)-1-(4-(1-aminoethyl)phenyl)-3-(4-methoxybenzyl)urea hydrochloride (6.18 g, 18.28 mmol, 90% yield) as a viscous, nearly colorless oil. The purity was estimated to be 70%. LCMS-APCI (POS.) m/z: 300.1 (M+H)⁺. ¹H NMR (400 MHz, DMSO-d₆) δ 9.14 (s, 1H), 8.40 (d, J=5.3 Hz, 3H), 7.45 (d, J=8.3 Hz, 2H), 7.36 (d, J=8.3 Hz, 2H), 7.23 (d, J=8.2 Hz, 2H), 6.89 (d, J=8.2 Hz, 3H), 4.29 (p, J=6.1 Hz, 1H), 4.22 (s, 2H), 3.73 (s, 3H), 1.49 (d, J=6.7 Hz, 3H).

Intermediates 2.2, 2.3, 2.4, 2.5, 2.6, and 2.7 were prepared in a similar manner as Intermediate 2.1, using the reagents provided in the table below in place of 4-methoxy benzyl isocyanate.

| Intermediate | Reagents | Structure, Name and Data |
|---|---|---|
| 2.2 | 4-Chloro benzyl isocyanate | 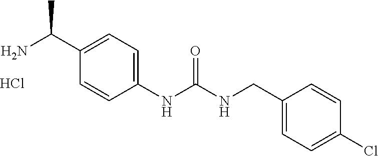 [SMILES: Cl ; C[C@H](N)c1ccc(NC(=O)NCc2ccc(Cl)cc2)cc1] (S)-1-(4-(1-aminoethyl)phenyl)-3-(4- chlorobenzyl)urea hydrochloride. LCMS-ESI (POS.) m/z: 304.0 (M + H)+. |
| 2.3 | 4-Chloro benzyl isocyanate | 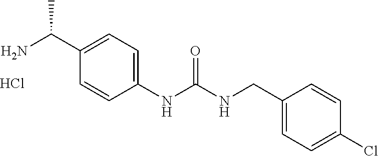 [SMILES: Cl ; C[C@@H](N)c1ccc(NC(=O)NCc2ccc(Cl)cc2)cc1] (R)-1-(4-(1-aminoethyl)phenyl)-3-(4- chlorobenzyl)urea hydrochloride. LCMS-ESI (POS.) m/z: 304.0 (M + H)+. |
| 2.4 | 4-methoxy benzyl isocyanate | 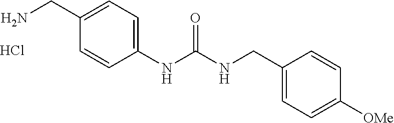 [SMILES: Cl ; COc1ccc(CNC(=O)Nc2ccc(CN)cc2)cc1] 1-(4-(aminom ethyl)phenyl)-3-(4- methoxybenzyl)urea hydrochloride. LCMS-ESI (POS.) m/z: 286.1 (M + H)+. |
| 2.5 | 4-chloro benzyl isocyanate | 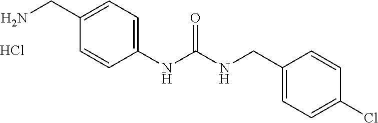 [SMILES: Cl ; NCc1ccc(NC(=O)NCc2ccc(Cl)cc2)cc1] 1-(4-(aminomethyl)phenyl)-3-(4- chlorobenzyl)urea hydrochloride. LCMS-ESI (POS.) m/z: 290.0 (M + H)+. |
| 2.6 | 4-fluoro benzyl isocyanate | 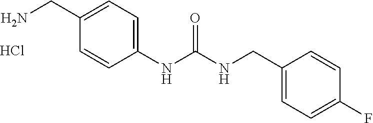 [SMILES: Cl ; NCc1ccc(NC(=O)NCc2ccc(F)cc2)cc1] (S)-1-(4-(1-aminoethyl)phenyl)-3-(4- fluorobenzyl)urea hydrochloride. LCMS-ESI (POS.) m/z: 274.0 (M + H)+. |
| 2.7 | 4-methoxy benzyl isocyanate | 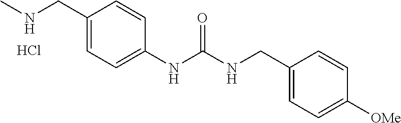 [SMILES: CNCc1ccc(NC(=O)NCc2ccc(OC)cc2)cc1 ; Cl] 1-(4-methoxybenzyl)-3-(4- ((methylamino)methyl)phenyl)urea hydrochloride. LCMS-ESI (POS.) m/z: 300.1 (M + H)+. |
| 2.8 | 4-chloro benzyl isocyanate | 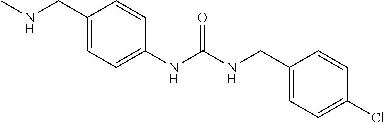 [SMILES: CNCc1ccc(NC(=O)NCc2ccc(Cl)cc2)cc1] 1-(4-chlorobenzyl)-3-(4- ((methylamino)methyl)phenyl)urea hydrochloride. LCMS-ESI (POS.) m/z: 304.1 (M + H)+. |

### Example C

### Synthesis of Intermediates 3.1, 3.2, and 3.3

### Step 1: Preparation of methyl 4-(3-(4-methoxybenz 1 ureido benzoate (Intermediate 3-a)

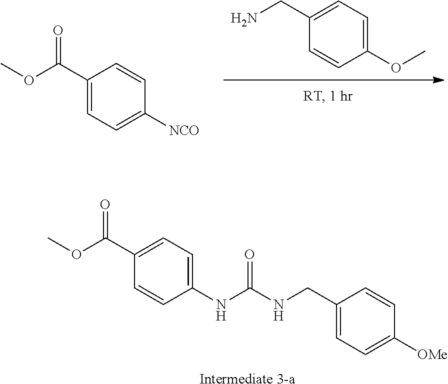

To a suspension of methyl 4-isocyanatobenzoate (10.0 g, 56.4 mmol) in methylene chloride (56.4 mL, 1M) was added (4-methoxyphenyl)methanamine (7.74 g, 56.4 mmol) dropwise at 0° C. The reaction was gradually warmed to rt and stirred at room temperature for 60 minutes and the reaction progress was monitored with LC/MS. The reaction became homogenous followed by the white solid precipitation. The solution was then filtered, and the filter cake was washed with excess methylene chloride and dried to afford crude Intermediate 3-a (17.4 g, 55.2 mmol, 98% yield) as an off-white solid set up. LCMS-APCI (POS.) m/z: 315.2 (M+H)+. 1H NMR (400 MHz, DMSO-d6) δ 8.96 (s, 1H), 7.85 (d, J=8.6 Hz, 2H), 7.54 (d, J=8.8 Hz, 2H), 7.24 (d, J=8.5 Hz, 2H), 6.90 (d, J=8.5 Hz, 2H), 6.71 (t, J=5.9 Hz, 1H), 4.25 (d, J=5.7 Hz, 2H), 3.73 (s, 3H), 3.81 (s, 3H).

### Step 2: Preparation of 1-(4-(hydroxymethyl)phenyl)-3-(4-methoxybenzyl)urea (Intermediate 3-b)

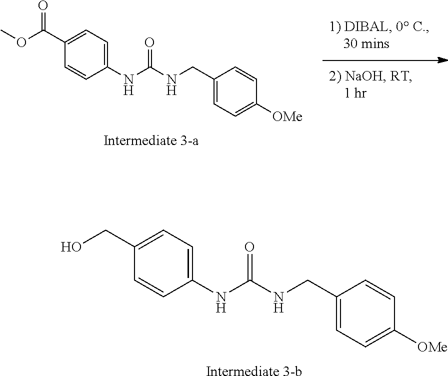 [SMILES: COC(=O)c1ccc(NC(=O)NCc2ccc(OC)cc2)cc1 ; "Intermediate 3-a" ; COc1ccc(CNC(=O)Nc2ccc(CO)cc2)cc1 ; "Intermediate 3-b"]

To a dry flask was added Intermediate 3-a (16.0 g, 50.9 mmol) in 120 mL dry methylene chloride and the suspension was cooled to 0° C. Next 1M DIBAL in methylene chloride (126 mL, 126 mmol) was added dropwise over 45 minutes and the reaction was stirred at 0° C. for an additional 30 minutes. The homogenous solution was allowed to warm to room temperature and then stirred for 4 h. The solution was subsequently cooled to 0° C. and quenched by MeOH (100 mL) dropwise and after exotherm subsided 300 mL of methylene chloride and 200 mL of sodium hydroxide solution (1M) added and the mixture was stirred for another 60 minutes at room temperature. Then the organic layer was separated and the aqueous layer was extracted with (5:1 methylene chloride-isopropanol, 300 mL) The combined organic layer was washed with brine and dried over magnesium sulfate, filtered, and evaporated to produce Intermediate 3-b as a white solid (14.2 g, 49.8 mmol, 99% yield). The crude product was taken through the following oxidation stage with further purification. LCMS-APCI (POS.) m/z: 287.2 (M+H)⁺. ¹H NMR (400 MHz, DMSO-d₆) δ 8.61 (s, 1H), 7.35 (d, J=8.0 Hz, 2H), 7.24 (d, J=8.2 Hz, 2H), 7.17 (d, J=8.1 Hz, 2H), 6.90 (d, J=8.2 Hz, 2H), 6.61 (t, J=5.9 Hz, 1H), 5.02 (t, J=5.7 Hz, 1H), 4.40 (d, J=5.5 Hz, 2H), 4.22 (d, J=5.7 Hz, 2H), 3.74 (s, 3H).

### Step 3: Preparation of 1-(4-formylphenyl)-3-(4-methoxybenzyl)urea (Intermediate 3.1)

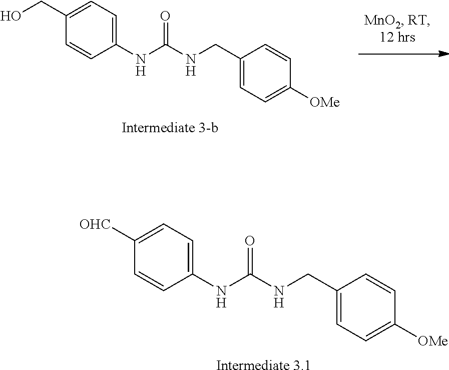 [SMILES: COc1ccc(CNC(=O)Nc2ccc(CO)cc2)cc1 ; "Intermediate 3-b" ; COc1ccc(CNC(=O)Nc2ccc(C=O)cc2)cc1 ; "Intermediate 3.1"]

To a suspension of Intermediate 3-b (14.0 g, 48.8 mmol) in methylene chloride-isopropanol (20:1, 250 mL, 0.2 M) was added manganese dioxide (44.2 g, 508 mmol) at room temperature. The resulting suspension was allowed to stir for 12 hours at rt. The solution was then filtered over celite. The filter cake was washed with isopropanol and the mother liquor was concentrated to provide Intermediate 3.1 (13.2 g, 46.5 mmol) as a light yellow solid set up. LCMS-APCI (POS.) m/z: 285.2 (M+H)⁺. ¹H NMR (400 MHz, DMSO-d₆) δ 9.81 (s, 1H), 9.15 (s, 1H), 7.78 (dd, J=8.6, 2.7 Hz, 2H), 7.62 (dd, J=8.6, 2.7 Hz, 2H), 7.24 (dd, J=8.5, 2.8 Hz, 2H), 6.90 (dd, J=8.6, 2.7 Hz, 2H), 6.87-6.77 (m, 1H), 4.43 (dd, J=8.5, 2.8 Hz, 2H), 3.74 (s, 3H).

Intermediates 3.2 and 3.3 were prepared in a similar manner as Intermediate 2.1, using the reagents provided in the table below in place of (4-methoxyphenyl)methanamine.

| Intermediate | Reagents | Structure, Name and Data |
|---|---|---|
| 3.2 | (4-chlorophenyl)methanamine | 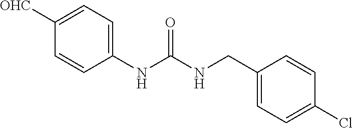 [SMILES: O=Cc1ccc(NC(=O)NCc2ccc(Cl)cc2)cc1] 1-(4-formylphenyl)-3-(4-chlorobenzyl)urea. LCMS-ESI (POS.) m/z: 289.2 (M + H)+. |
| 3.3 | (4-fluorophenyl)methanamine | 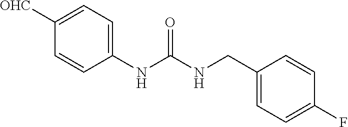 [SMILES: O=Cc1ccc(NC(=O)NCc2ccc(F)cc2)cc1] 1-(4-formylphenyl)-3-(4-fluorobenzyl)urea. LCMS-ESI (POS.) m/z: 273.2 (M + H)+. |

### Example D

### Synthesis of Intermediates 4.1 and 4.2

### Step 1: Preparation of phenyl (4-chlorobenzyl)carbamate (Intermediate 4.1)

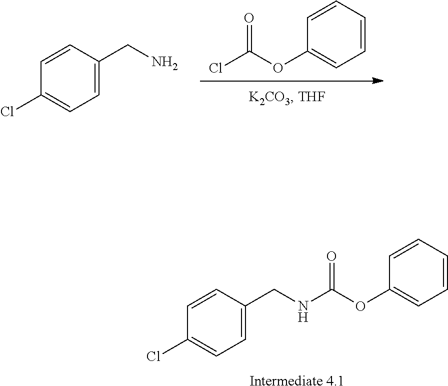 [SMILES: NCc1ccc(Cl)cc1 ; O=C(Cl)Oc1ccccc1 ; O=C(NCc1ccc(Cl)cc1)Oc1ccccc1 ; "Intermediate 4.1"]

To a solution of 1-(4-chlorophenyl)methanamine (2.00 g, 14.124 mmol, 1.00 equiv) in THF(30 mL) were added phenyl carbonochloridate (2.43 g, 15.537 mmol, 1.1 equiv) and K₂CO₃ (2.93 g, 21.186 mmol, 1.5 equiv). The resulting mixture was stirred at r.t. for 3 h, filtered to remove solids, and the filtrate was concentrated and purified by silica gel column chromatography, eluted with PE/EtOAc (5:1) to afford 3.6 g of phenyl N-[(4-chlorophenyl)methyl]carbamate (95%) as a white solid. LRMS (ES) m/z 262[M+H].

Intermediate 4.2 was prepared in a similar manner as Intermediate 4.1, using (4-methoxyphenyl)methanamine in place of (4-chlorophenyl)methanamine.

| Intermediate | Reagents | Structure, Name and Data |
|---|---|---|
| 4.2 | (4-methoxyphenyl)methanamine | 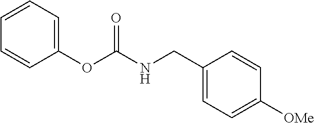 [SMILES: COc1ccc(CNC(=O)Oc2ccccc2)cc1] |
| | | Phenyl (4-methoxybenzyl)carbamate. |
| | | LCMS-ESI (POS.) m/z: 258 (M + H)+. |

### Example E

### Synthesis of 4-(1-(methylsulfonyl)ethyl)aniline (Intermediate 5.0)

### Step 1: Preparation of 1-((methylsulfonyl)methyl)-4-nitrobenzene (Intermediate 5-a)

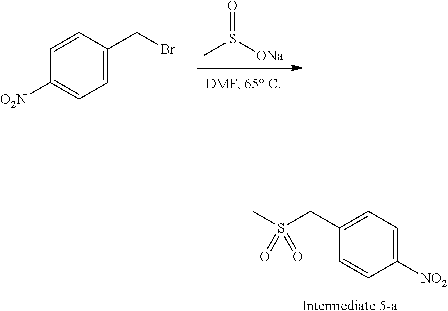

To a solution of 1-(bromomethyl)-4-nitrobenzene (1 g, 4.629 mmol, 1 equiv) in DMF (10 mL) was added sodium methanesulfinate (712 mg, 6.975 mmol, 1.51 equiv). The resulting mixture was stirred at 65° C. for 0.5 h, cooled to r.t., added water (20 mL) and the mixture was extracted with EtOAc (20 mL) twice. The combined organic layers were washed twice with brine (20 mL), dried over anhydrous Na₂SO₄, concentrated under reduced pressure to give 1 g of 1-(methanesulfonylmethyl)-4-nitrobenzene as a yellow solid. (No LCMS signal, H-NMR confirmed). 1H NMR (300 MHz, DMSO-d6) δ 8.34-8.23 (m, 2H), 7.76-7.65 (m, 2H), 4.73 (s, 2H), 2.99 (s, 3H).

### Step 2: Preparation of 1-(methylsulfonyl)ethyl)-4-nitrobenzene (Intermediate 5-b)

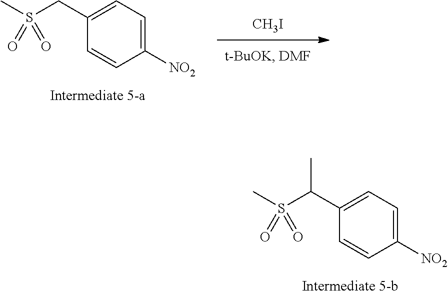

To a solution of 1-(methanesulfonylmethyl)-4-nitrobenzene (850 mg, 3.949 mmol, 1 equiv) in DMF (10 mL) was added t-BuOK (531 mg, 4.732 mmol, 1.20 equiv). After stirring at r.t. for 1 h, the mixture was added iodomethane (560 mg, 3.945 mmol, 1.00 equiv). The resulting mixture was stirred at r.t. for 1 h, added water (20 mL). The mixture was extracted with EtOAc (20 mL) twice. The combined organic layers were washed twice with brine (20 mL), dried over anhydrous Na₂SO₄, concentrated under reduced pressure to give 950 mg of 1-(1-methanesulfonylethyl)-4-nitrobenzene as a yellow oil. No LCMS signal. H-NMR analysis indicated it was the desired product. 1H NMR (400 MHz, DMSO-d6) δ 8.33-8.23 (m, 2H), 7.79-7.67 (m, 2H), 4.82 (q, J=7.1 Hz, 1H), 2.91 (s, 3H), 1.69 (d, J=7.1 Hz, 3H).

### Step 3: Preparation of 4-(1-(methylsulfonyl)ethyl)aniline (Intermediate 5.0)

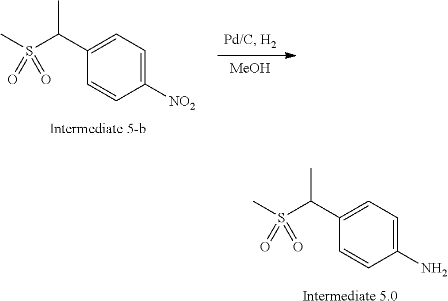

To a solution of 1-(1-methanesulfonylethyl)-4-nitrobenzene (950 mg, 4.144 mmol, 1 equiv) in methanol (10 mL) was added Pd/C (467 mg, 50% w/w). The resulting mixture was stirred at r.t. for 1 h under hydrogen atmosphere, filtered to remove solids and the filtrate was concentrated under reduced pressure to give 700 mg of 4-(1-methanesulfonylethyl)aniline as a yellow oil. LRMS (ES) m/z 200[M+H].

### Example F

### Synthesis of 3-(4-aminophenyl)thietane 1,1-dioxide trifluoroacetate salt (Intermediate 6.0)

### Step 1: Preparation of diethyl 2-(4-nitrophenyl)malonate (Intermediate 6-a)

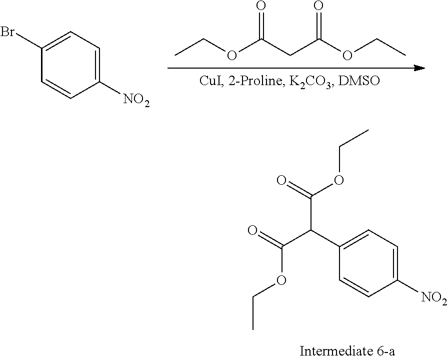

To a solution of 1-bromo-4-nitrobenzene (5 g, 24.752 mmol, 1 equiv) in DMSO (50 mL) were added 1,3-diethyl propanedioate (12 g, 74.921 mmol, 3.03 equiv), CuI (473 mg, 2.484 mmol, 0.10 equiv), L-Proline (572 mg, 4.968 mmol, 0.20 equiv) and K₂CO₃ (13.7 g, 99.128 mmol, 4.00 equiv). The mixture was stirred at 90° C. for 2 days under nitrogen atmosphere, cooled to r.t., added water (100 mL) and extracted with EtOAc (100 mL) twice. The combined organic layers were washed twice with brine (100 mL), dried over anhydrous Na₂SO₄, concentrated under reduced pressure, and purified by silica gel column chromatography, eluted with PE/EtOAc (20:1) to afford 4.7 g of 1,3-diethyl 2-(4-nitrophenyl)propanedioate as a yellow oil. LRMS (ES) m/z 282 (M+H).

### Step 2: Preparation of diethyl 2-(4-aminophenyl)malonate (Intermediate 6-b)

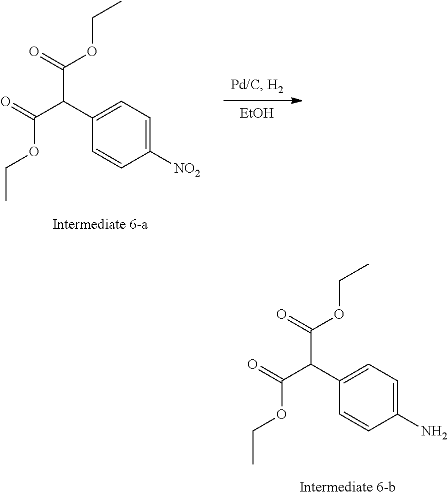 [SMILES: "EtOH" ; CCOC(=O)C(C(=O)OCC)c1ccc([N+](=O)[O-])cc1 ; CCOC(=O)C(C(=O)OCC)c1ccc(N)cc1]

To a solution of 1,3-diethyl 2-(4-nitrophenyl)propanedioate (2.2 g, 7.822 mmol, 1 equiv) in ethanol (25 mL) was added Pd/C (1.10 g, 50% w/w). The resulting mixture was stirred at r.t. for 2 h under hydrogen atmosphere, filtered to remove the solids, and the filtrate was concentrated under reduced pressure to give 1.9 g of 1,3-diethyl 2-(4-aminophenyl)propanedioate (96.67%) as a yellow oil. LRMS (ES) m/z 252[M+H].

### Step 3: Preparation of diethyl 2-(4-((tert-butoxycarbonyl)amino)phenyl)malonate (Intermediate 6-c)

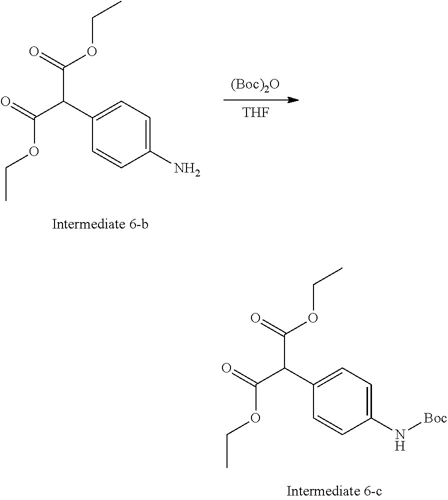 [SMILES: "(Boc)2O" ; CCOC(=O)C(C(=O)OCC)c1ccc(N)cc1 ; CCOC(=O)C(C(=O)OCC)c1ccc(NC(=O)OC(C)(C)C)cc1 ; "THF"]

To a solution of 1,3-diethyl 2-(4-aminophenyl)propanedioate (1 g, 3.96 mmol, 1 equiv) in THE (10 mL) was added di-tert-butyl dicarbonate (2.6 g, 11.4 mmol, 2.9 equiv). The resulting mixture was stirred at r.t. for 2 h, added water (30 mL) and the mixture was extracted with CH₂Cl₂ (30 mL) twice. The combined organic layers were washed twice with brine (30 mL), dried over anhydrous Na₂SO₄, concentrated under reduced pressure, and purified by silica gel column chromatography, eluted with PE/EtOAc (10:1) to afford 1 g of 1,3-diethyl 2-(4-[[(tert-butoxy)carbonyl]amino]phenyl)propanedioate as an off-white solid. LRMS (ES) m/z 296[M+H−56].

### Step 4: Preparation of tert-butyl (4-(1,3-dihydroxypropan-2-yl)phenyl)carbamate (Intermediate 6-d)

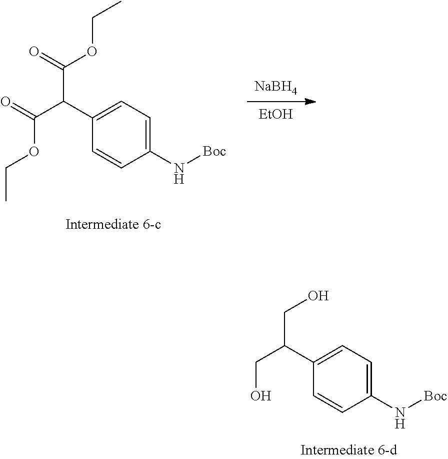 [SMILES: "EtOH" ; CCOC(=O)C(C(=O)OCC)c1ccc(NC(=O)OC(C)(C)C)cc1 ; "NaBH4" ; CC(C)(C)OC(=O)Nc1ccc(C(CO)CO)cc1]

To a solution of 1,3-diethyl 2-(4-[[(tert-butoxy)carbonyl]amino]phenyl)propanedioate (1 g, 2.846 mmol, 1 equiv) in ethanol (20 mL) was added NaBH₄ (1.08 g, 28.547 mmol, 10.03 equiv). The resulting mixture was stirred at r.t. for overnight, quenched with NH₄Cl.aq (10 mL) at 0° C., concentrated under vacuum to remove EtOH. The mixture was extracted with EtOAc (20 mL) twice. The combined organic layers were washed twice with brine (20 mL), dried over anhydrous Na₂SO₄, concentrated under reduced pressure, purified by silica gel column chromatography, eluted with CH₂Cl₂/MeOH (20:1) to afford 720 mg of tert-butyl N-[4-(1,3-dihydroxypropan-2-yl)phenyl]carbamate (94.64%) as an off-white solid. LRMS (ES) m/z 212[M+H−56].

### Step 5: Preparation of 2-(4-((tert-butoxycarbonyl)amino)phenyl)propane-1,3-diyl dimethanesulfonate (Intermediate 6-e)

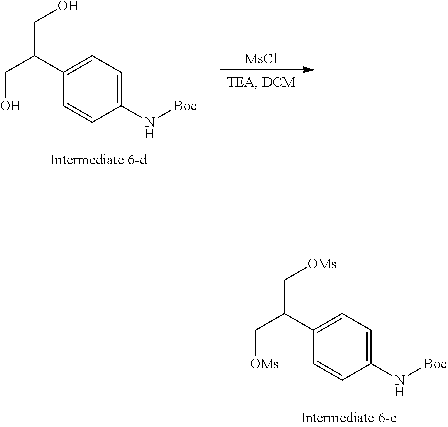 [SMILES: "MsCl" ; CC(C)(C)OC(=O)Nc1ccc(C(CO)CO)cc1 ; "TEA, DCM" ; CC(C)(C)OC(=O)Nc1ccc(C(COS(C)(=O)=O)COS(C)(=O)=O)cc1]

To a solution of tert-butyl N-[4-(1,3-dihydroxypropan-2-yl)phenyl]carbamate (670 mg, 2.506 mmol, 1 equiv) in DCM (10 mL) were added methanesulfonyl chloride (715 mg, 6.242 mmol, 2.49 equiv) and TEA (760 mg, 7.511 mmol, 3.00 equiv). The resulting mixture was stirred at r.t. for 2 h and poured into water (20 mL). The aqueous layer was extracted with CH₂Cl₂ (20 mL) twice. The combined organic layers were washed twice with brine (20 mL), dried over anhydrous Na₂SO₄, concentrated under reduced pressure to give 1.2 g of tert-butyl N-[4-[2-(methanesulfonyloxy)-1-[(methanesulfonyloxy)methyl]ethyl]phenyl]carbamate as a yellow solid. LRMS (ES) m/z 368[M+H−56].

### Step 6: Preparation of tert-butyl (4-(thietan-3-yl)phenyl)carbamate (Intermediate 6-f)

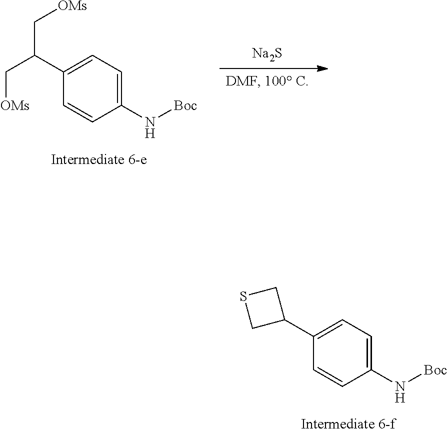 [SMILES: CC(C)(C)OC(=O)Nc1ccc(C(COS(C)(=O)=O)COS(C)(=O)=O)cc1 ; "Na2S" ; "DMF, 100° C." ; CC(C)(C)OC(=O)Nc1ccc(C2CSC2)cc1]

To a solution of tert-butyl N-[4-[2-(methanesulfonyloxy)-1-[(methanesulfonyloxy)methyl]ethyl]phenyl]carbamate (1.1 g, 2.597 mmol, 1 equiv) in DMF (10 mL) at r.t. was added Na₂S (122 mg, 1.564 mmol, 0.60 equiv). The resulting mixture was stirred at 100° C. for 5 h. The solution was then cooled to r.t. and poured into water (20 mL). The aqueous layer was extracted with EtOAc (30 mL) twice. The combined organic layers were washed twice with brine (30 mL), dried over anhydrous Na₂SO₄, concentrated under reduced pressure, purified by silica gel column chromatography, eluted with PE/EtOAc (5:1) to afford 270 mg of tert-butyl N-[4-(thietan-3-yl)phenyl]carbamate (39.17%) as a yellow solid. LRMS (ES) m/z 210[M+H−56].

### Step 7: Preparation of tert-butyl (4-(1,1-dioxidothietan-3-yl)phenyl)carbamate (Intermediate 6-g)

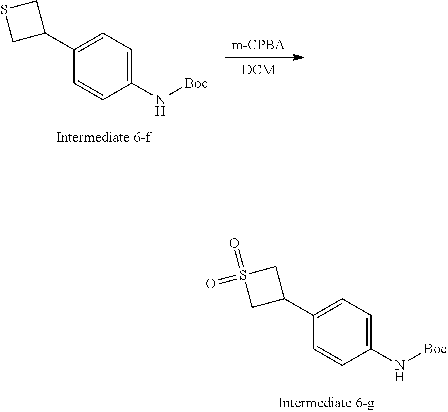

To a solution of tert-butyl N-[4-(thietan-3-yl)phenyl]carbamate (250 mg, 0.942 mmol, 1 equiv) in DCM (3 mL) at 0° C. was added m-CPBA (485 mg, 2.811 mmol, 2.98 equiv). The resulting mixture was stirred at r.t. for 2 h and added water (20 mL). The resulting mixture was extracted with CH₂Cl₂ (20 mL) twice. The combined organic layers were washed with Na₂S₂O₄(10 mL), NaHCO₃(10 mL) and twice with brine (20 mL), dried over anhydrous Na₂SO₄, concentrated under reduced pressure to give 290 mg of tert-butyl N-[4-(1,1-dioxo-1lambda6-thietan-3-yl)phenyl]carbamate as a yellow oil. LRMS (ES) m/z 242 [M+H−56].

### Step 8: Preparation of 3-(4-aminophenyl)thietane 1,1-dioxide trifluoroacetate salt (Intermediate 6.0)

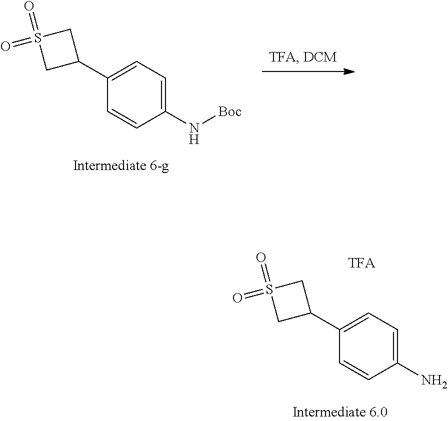

To a solution of tert-butyl N-[4-(1,1-dioxo-1lambda6-thietan-3-yl)phenyl]carbamate (290 mg, 0.975 mmol, 1 equiv) in DCM (3 mL) was added TFA (0.5 mL). The resulting mixture was stirred at r.t. for 2 h, concentrated under reduced pressure to give 190 mg of 3-(4-aminophenyl)thietane 1,1-dioxide trifluoroacetate salt as a brown solid. LRMS (ES) m/z 298[M+H].

### Example G

### Synthesis of 2-(4-aminophenyl)tetrahydrothiophene 1,1-dioxide (Intermediate 7.0)

### Step 1: Preparation of 2-(4-nitrophenyl)tetrahydrothiophene 1,1-dioxide (Intermediate 7-a)

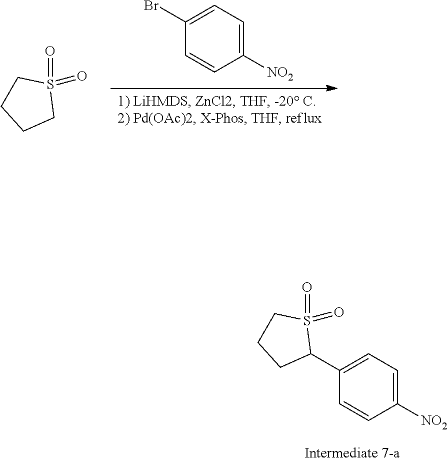

To a solution of tetrahydrothiophene 1,1-dioxide (2 g, 16.643 mmol, 1.00 equiv) in THF (20.00 mL) at −20° C. was added LiHMDS (25.00 mL, 25.000 mmol, 1.50 equiv) dropwise over a period of 20 min under nitrogen atmosphere. After stirring at r.t. for 0.5 h under nitrogen atmosphere, the mixture was added ZnCl₂ (3.35 g, 24.575 mmol, 1.48 equiv) at −20° C. The mixture was stirred at r.t. for 1 h. To the above mixture were added 1-bromo-4-nitrobenzene (2.35 g, 11.650 mmol, 0.70 equiv), Pd(OAc)₂ (187.00 mg, 0.833 mmol, 0.05 equiv) and X-Phos (795.00 mg, 1.668 mmol, 0.10 equiv). The mixture was stirred at 65° C. for 12 h under nitrogen atmosphere, cooled to r.t., quenched with aqueous NH₄Cl (20 mL) and HCl (1 mol/L, 5 mL) and extracted with CH₂Cl₂ (50 mL) twice. The combined organic layers were washed twice with brine (50 mL), dried over anhydrous Na₂SO₄, concentrated under reduced pressure, purified by silica gel column chromatography, eluted with PE/EtOAc (3:2) to afford 1.1 g of 2-(4-nitrophenyl)tetrahydrothiophene 1,1-dioxide (27.40%) as a brown solid. No LCMS signal.

### Step 2: Preparation of 2-(4-aminophenyl)tetrahydrothiophene 1,1-dioxide (Intermediate 7.0)

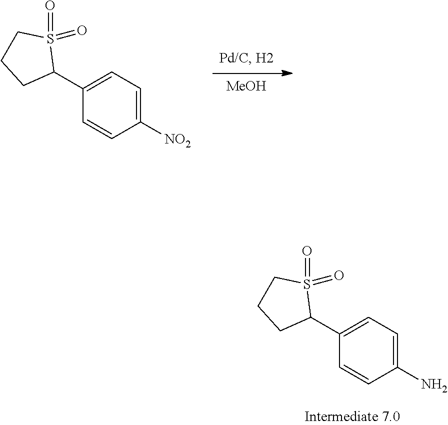 [SMILES: "MeOH" ; O=[N+]([O-])c1ccc(C2CCCS2(=O)=O)cc1 ; "Pd/C, H2" ; Nc1ccc(C2CCCS2(=O)=O)cc1]

To a solution of 2-(4-nitrophenyl)tetrahydrothiophene 1,1-dioxide (1.10 g, 4.559 mmol, 1.00 equiv) in methanol (11 mL) was added Pd/C (550.00 mg, 50% w/w). The resulting mixture was stirred at r.t. for overnight under hydrogen atmosphere, filtered to remove solids, and the filtrate was concentrated under reduced pressure to afford 800 mg of 2-(4-aminophenyl)tetrahydrothiophene 1,1-dioxide (83.05%) as a yellow solid. LRMS (ES) m/z 212[M+H].

### Example H

### Synthesis of 3-(4-aminophenyl)tetrahydrothiophene 1,1-dioxide trifluoroacetate salt (Intermediate 8.0)

### Step 1: Preparation of tert-butyl (4-iodophenyl)carbamate (Intermediate 8-a)

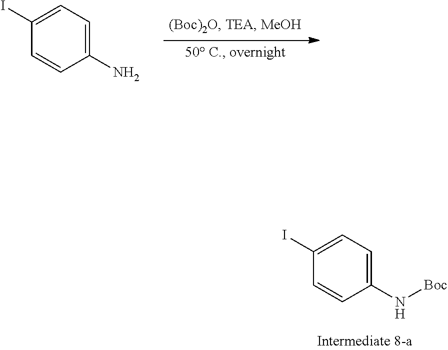 [SMILES: Nc1ccc(I)cc1 ; "(Boc)2O, TEA, MeOH" ; "50° C., overnight" ; CC(C)(C)OC(=O)Nc1ccc(I)cc1]

To a solution of 4-iodoaniline (1 g, 4.566 mmol, 1 equiv) in MeOH (20 mL) were added (Boc)₂O (2 g, 0.009 mmol, 2.01 equiv) and TEA (2 mL). The resulting mixture was stirred at 50° C. for overnight, cooled to r.t., concentrated under vacuum, added water (50 mL). The mixture was extracted with EtOAc (50 mL) twice. The combined organic layers were washed twice with brine (50 mL), dried over anhydrous Na₂SO₄, concentrated under reduced pressure, purified by silica gel column chromatography, eluted with PE/EtOAc (30:1) to afford 650 mg of tert-butyl N-(4-iodophenyl)carbamate (650 mg, 44.61%) as an off-white solid. LRMS (ES) m/z 264[M+H−56].

### Step 2: Preparation of tert-butyl (4-(1,1-dioxido-2,5-dihydrothiophen-3-yl)phenyl)carbamate (Intermediate 8-b)

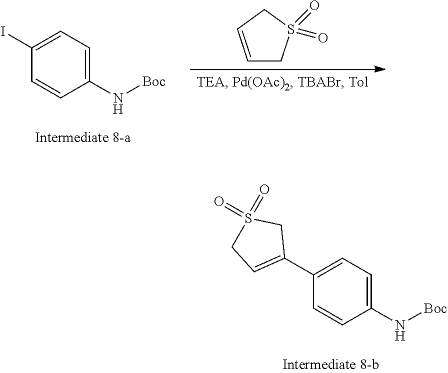

To a solution of tert-butyl N-(4-iodophenyl)carbamate (650 mg, 2.037 mmol, 1 equiv) in Toluene (10 mL) were added 2,5-dihydro-1lambda6-thiophene-1,1-dione (264 mg, 2.234 mmol, 1.10 equiv), Pd(OAc)₂ (91 mg, 0.405 mmol, 0.20 equiv), TBABr (654 mg, 2.029 mmol, 1.00 equiv) and TEA (410 mg, 4.052 mmol, 1.99 equiv). The resulting mixture was stirred at r.t. for 3 days under nitrogen atmosphere and at 80° C. for 3 h, cooled to r.t., added water (20 mL). The mixture was extracted with EtOAc (30 mL) twice. The combined organic layers were washed twice with brine (30 mL), dried over anhydrous Na₂SO₄, concentrated under reduced pressure, purified by silica gel column chromatography, eluted with PE/EtOAc (3:2) to afford 430 mg of tert-butyl N-[4-(1,1-dioxo-2,5-dihydro-1lambda6-thiophen-3-yl)phenyl]carbamate (68.24%) as a brown solid. LRMS (ES) m/z 254[M+H−56].

### Step 3: Preparation of tert-butyl (4-(1,1-dioxidotetrahydrothiophen-3-yl)phenyl)carbamate (Intermediate 8-c)

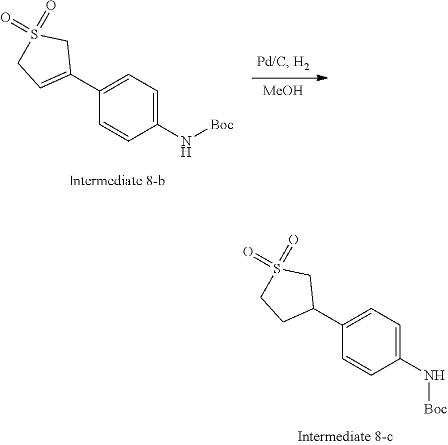 [SMILES: "MeOH" ; CC(C)(C)OC(=O)Nc1ccc(C2=CCS(=O)(=O)C2)cc1 ; "Pd/C, H2" ; CC(C)(C)OC(=O)Nc1ccc(C2CCS(=O)(=O)C2)cc1]

To a solution of tert-butyl N-[4-(1,1-dioxo-2,5-dihydro-1lambda6-thiophen-3-yl)phenyl]carbamate (430 mg, 1.390 mmol, 1 equiv) in methanol (10 mL) was added Pd/C (215 mg, 50% w/w). The resulting mixture was stirred at r.t. for 1 h under hydrogen atmosphere, filtered to remove solids, and the filtrate was concentrated under reduced pressure to afford 390 mg of tert-butyl N-[4-(1,1-dioxo-1lambda6-thiolan-3-yl)phenyl]carbamate (90.11%) as a brown solid. LRMS (ES) m/z 256[M+H].

### Step 4: Preparation of 3-(4-aminophenyl)tetrahydrothiophene 1,1-dioxide trifluoroacetate salt (Intermediate 8.0)

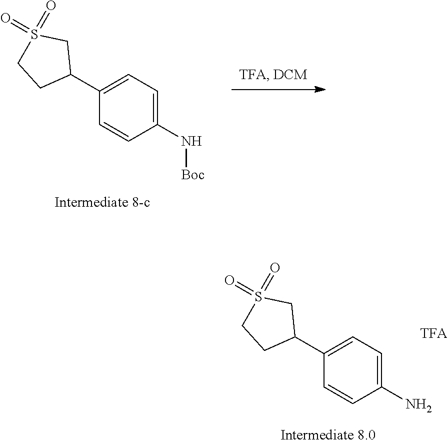

To a solution of tert-butyl N-[4-(1,1-dioxo-1lambda6-thiolan-3-yl)phenyl]carbamate (390 mg, 1.252 mmol, 1 equiv) in DCM (5 mL) was added TFA (1 mL). The resulting mixture was stirred at r.t. for 2 h, concentrated under reduced pressure, diluted with water (10 mL) and adjusted pH to 8 with Na₂CO₃ aq. The aqueous layer was extracted with EA (10 ml) twice. The combined organic layers were washed twice with brine (10 mL), dried over Na₂SO₄, concentrated under reduced pressure to give 260 mg of 3-(4-aminophenyl)tetrahydrothiophene 1,1-dioxide trifluoroacetate salt as a brown oil. LRMS (ES) m/z 212[M+H].

### Example I

### Synthesis of 4-(4-aminophenyl)tetrahydro-2H-thiopyran 1,1-dioxide (Intermediate 9.0)

### Step 1: Preparation of 3,6-dihydro-2H-thiopyran-4-yl trifluoromethanesulfonate (Intermediate 9-a)

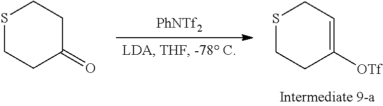 [SMILES: "PhNTf2" ; O=C1CCSCC1 ; "LDA, THF, -78° C." ; O=S(=O)(OC1=CCSCC1)C(F)(F)F]

To a solution of LDA (8.5 mL, 17.0 mmol, 1.10 equiv) in THF (20 mL) at −78° C. was added a solution of thian-4-one (1.8 g, 15.493 mmol, 1 equiv) in THF (5 mL) dropwise over a period of 10 min under argon atmosphere. After stirring at r.t. for 0.5 h under argon atmosphere, the mixture at −78° C. was added a solution of 1,1,1-trifluoro-N-phenyl-N-trifluoromethanesulfonylmethanesulfonamide (6.09 g, 17.047 mmol, 1.10 equiv) in THF(10 mL) dropwise over a period of 10 min. The resulting mixture was stirred at r.t. for 0.5 h under argon atmosphere, quenched with water (100 mL) at 0° C. and extracted with EtOAc (200 mL) twice. The combined organic layers were washed twice with brine (100 mL), dried over anhydrous Na₂SO₄, concentrated under reduced pressure, purified by silica gel column chromatography, eluted with PE/EtOAc (99:1) to afford 2.5 g of 3,6-dihydro-2H-thiopyran-4-yl trifluoromethanesulfonate as a yellow oil. LRMS (ES) m/z 249[M+H].

### Step 2: Preparation of 4-(4-nitrophenyl)-3,6-dihydro-2H-thiopyran (Intermediate 9-b)

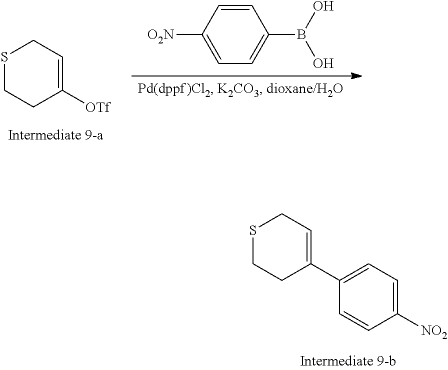

To a solution of 3,6-dihydro-2H-thiopyran-4-yl trifluoromethanesulfonate (2.4 g, 9.668 mmol, 1 equiv) in dioxane (20 mL) and H₂O (10 mL) were added (4-nitrophenyl)boronic acid (1.94 g, 11.602 mmol, 1.20 equiv), Pd(dppf)Cl₂CH₂Cl₂ (1.58 g, 1.934 mmol, 0.20 equiv) and K₂CO₃ (2.66 g, 19.34 mmol, 2 equiv). The resulting mixture was stirred at 85° C. for 3 h under nitrogen atmosphere, cooled to r.t., and added water (200 mL). The resulting mixture was extracted with EtOAc (200 mL) twice. The combined organic layers were washed twice with brine (200 mL), dried over anhydrous Na₂SO₄, concentrated under reduced pressure, purified by silica gel column chromatography, eluted with PE/EtOAc (20:1) to afford 1 g of 4-(4-nitrophenyl)-3,6-dihydro-2H-thiopyran (46.74%) as a yellow solid. LRMS (ES) m/z 222[M+H].

### Step 3: Preparation of 4-(4-nitrophenyl)-3,6-dihydro-2H-thiopyran 1,1-dioxide (Intermediate 9-c)

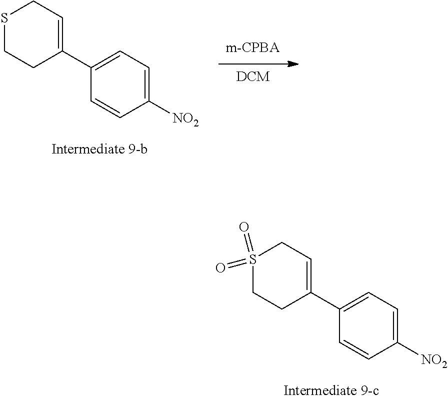

To a solution of 4-(4-nitrophenyl)-3,6-dihydro-2H-thiopyran (700 mg, 3.164 mmol, 1 equiv) in DCM (15 mL) at −78° C. was added m-CPBA (1.6 g, 9.5 mmol, 3 equiv). The resulting mixture was stirred at r.t. for 3 h, poured into water (20 mL). The aqueous layer was extracted with CH₂Cl₂ (30 mL) twice. The combined organic layers were washed with Na₂SO₃(aq. 10 mL), NaHCO₃ (aq.10 mL) and twice with brine (20 mL), dried over anhydrous Na₂SO₄, concentrated under reduced pressure to give 650 mg of 4-(4-nitrophenyl)-3,6-dihydro-2H-thiopyran 1,1-dioxide as a yellow solid. LRMS (ES) m/z 254[M+H].

### Step 4: Preparation of 4-(4-aminophenyl)tetrahydro-2H-thiopyran 1,1-dioxide (Intermediate 9.0)

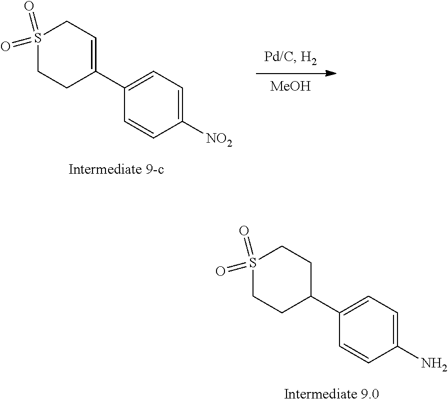 [SMILES: "MeOH" ; O=[N+]([O-])c1ccc(C2=CCS(=O)(=O)CC2)cc1 ; Nc1ccc(C2CCS(=O)(=O)CC2)cc1]

To a solution of 4-(4-nitrophenyl)-3,6-dihydro-2H-thiopyran 1,1-dioxide (650 mg, 2.559 mmol, 1 equiv) in methanol (8 mL) and THE (8 mL) was added Pd/C (325 mg, 50% w/w). The resulting mixture was stirred at r.t. for overnight under hydrogen atmosphere, filtered to remove solids, and the filtrate was concentrated under reduced pressure to give 400 mg of 4-(4-aminophenyl)tetrahydro-2H-thiopyran 1,1-dioxide as a brown solid. LRMS (ES) m/z 226 [M+H].

### Example J

### Synthesis of 4-(4-aminophenyl)-4-methyltetrahydro-2H-thiopyran 1,1-dioxide (Intermediate 10.0)

### Step 1: Preparation of ethyl (Z)-2-cyano-3-(4-nitrophenyl)but-2-enoate (Intermediate 10-a)

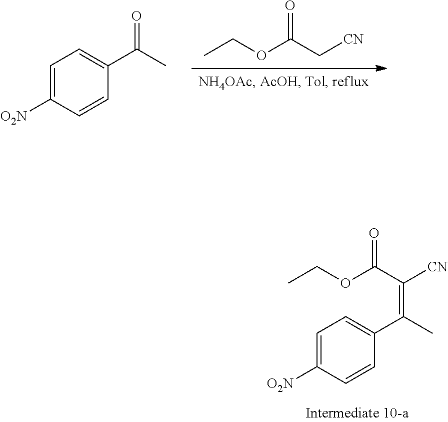

To a solution of 1-(4-nitrophenyl)ethan-1-one (2 g, 12.110 mmol, 1 equiv) in AcOH (6 mL) and toluene (40 mL) were added ethyl 2-cyanoacetate (1.37 g, 12.111 mmol, 1.00 equiv) and NH₄OAc (187 mg, 2.426 mmol, 0.20 equiv). The resulting mixture was stirred at 110° C. for overnight, cooled to r.t., and poured into water (50 mL). The resulting mixture was extracted with EtOAc (50 mL) twice. The combined organic layers were washed twice with brine (50 mL), dried over anhydrous Na₂SO₄, concentrated under reduced pressure, purified by silica gel column chromatography, eluted with PE/EtOAc (10:1) to afford 1.7 g of ethyl (Z)-2-cyano-3-(4-nitrophenyl)but-2-enoate (53.94%) as a yellow solid. LRMS (ES) m/z 261 (M+H).

### Step 2: Preparation of 4-methyl-4-(4-nitrophenyl)-2,6-dioxopiperidine-3,5-dicarbonitrile (Intermediate 10-b)

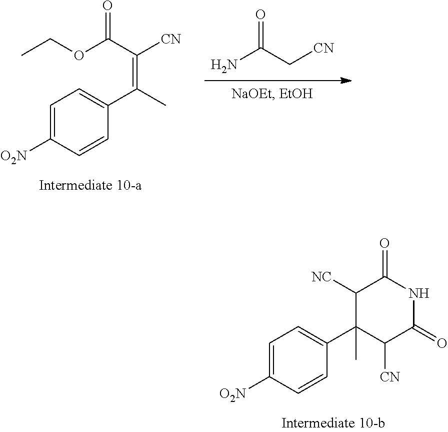

To a solution of NaOEt (2 g, 6.176 mmol, 1.00 equiv, 21%) in EtOH (30 mL) at 0° C. was added 2-cyanoacetamide (517 mg, 6.149 mmol, 1.00 equiv) dropwise over a period of 5 min. After stirring at r.t. for 15 min, ethyl (2Z)-2-cyano-3-(4-nitrophenyl)but-2-enoate (1.6 g, 6.148 mmol, 1 equiv) was added. The resulting mixture was stirred at r.t. for 4 h, concentrated under reduced pressure. The residue was dissolved in water (20 mL) and the mixture was acidified to pH 1 with HCl (aq.4 mol/L, ˜5 mL). The precipitated solids were collected by filtration and dried under reduced pressure to give 1.2 g of 4-methyl-4-(4-nitrophenyl)-2,6-dioxopiperidine-3,5-dicarbonitrile (65.44%) as a yellow solid. No LCMS signal. H-NMR confirmed. 1H NMR (400 MHz, DMSO-d6) δ 12.43 (s, 1H), 8.42-8.34 (m, 3H), 8.02-7.94 (m, 2H), 5.43 (s, 2H), 1.76 (s, 3H).

### Step 3: Preparation of 3-methyl-3-(4-nitrophenyl)pentanedioic acid (Intermediate 10-c)

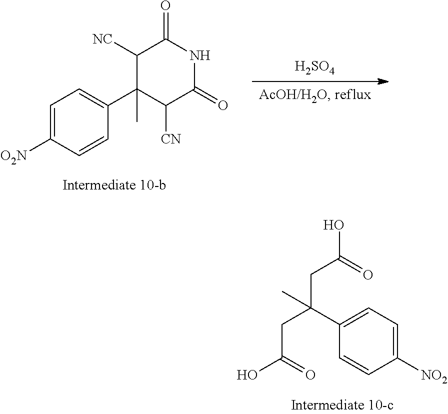 [SMILES: CC1(c2ccc([N+](=O)[O-])cc2)C(C#N)C(=O)NC(=O)C1C#N ; "H2SO4" ; "AcOH/H2O, reflux" ; CC(CC(=O)O)(CC(=O)O)c1ccc([N+](=O)[O-])cc1]

To a solution of 4-methyl-4-(4-nitrophenyl)-2,6-dioxopiperidine-3,5-dicarbonitrile (1.1 g, 3.688 mmol, 1 equiv) in H2O (9 mL) at 0° C. were added sulfuric acid (9 mL) and AcOH (6 mL) dropwise over a period of 15 min. The resulting mixture was stirred at 100° C. for 2 days, cooled to r.t., diluted with ice-cold water (30 mL) and extracted with EtOAc (30 mL) twice. The combined organic layers were washed twice with brine (50 mL) twice, dried over anhydrous Na₂SO₄, concentrated under reduced pressure to give 1.2 g of 3-methyl-3-(4-nitrophenyl)pentanedioic acid as a brown semi-solid. LRMS (ES) m/z 268 (M+H).

### Step 4: Preparation of 3-methyl-3-(4-nitrophenyl)pentane-1,5-diol (Intermediate 10-d)

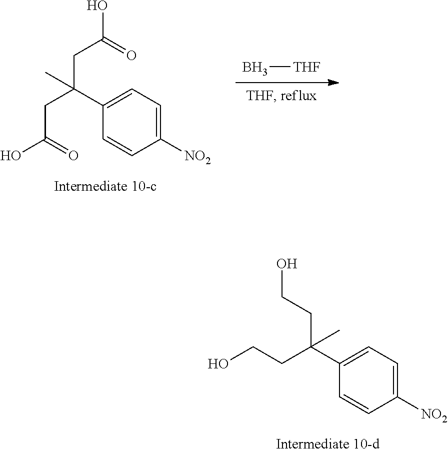

To a solution of 3-methyl-3-(4-nitrophenyl)pentanedioic acid (1.1 g, 4.1 mmol, 1 equiv) in THF (10 mL) at 0° C. was added BH₃-THF (1 mol/L in THF, 41 mL, 41 mmol, 10 equiv) dropwise over a period of 15 min. The resulting mixture was stirred at 70° C. for 1.5 h, cooled to r.t., quenched with water (30 mL) at 0° C., and extracted with EtOAc (30 mL) twice. The combined organic layers were washed twice with brine (30 mL), dried over anhydrous Na₂SO₄, concentrated under reduced pressure to give 720 mg of 3-methyl-3-(4-nitrophenyl)pentane-1,5-diol (82.06%) as a brown oil. LRMS (ES) m/z 240 (M+H).

### Step 5: Preparation of 3-methyl-3-(4-nitrophenyl)pentane-1,5-diyl dimethanesulfonate (Intermediate 10-e)

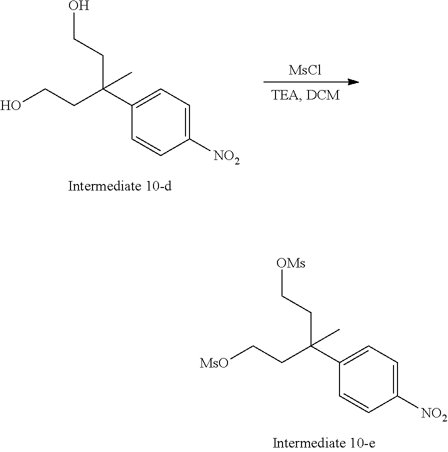 [SMILES: "MsCl" ; CC(CCO)(CCO)c1ccc([N+](=O)[O-])cc1 ; "TEA, DCM" ; CC(CCOS(C)(=O)=O)(CCOS(C)(=O)=O)c1ccc([N+](=O)[O-])cc1]

To a solution of 3-methyl-3-(4-nitrophenyl)pentane-1,5-diol (720 mg, 3.009 mmol, 1 equiv) in DCM (10 mL) at 0° C. were added TEA (912 mg, 9.013 mmol, 3.00 equiv) and methanesulfonyl chloride (859 mg, 7.500 mmol, 2.49 equiv) dropwise. The resulting mixture was stirred at r.t. for 2 h, poured into water (10 mL). The aqueous layer was extracted with CH₂Cl₂ (10 mL) twice. The combined organic layers were washed twice with brine (10 mL), dried over anhydrous Na₂SO₄, concentrated under reduced pressure, purified by silica gel column chromatography, eluted with PE/EtOAc (3:2) to afford 410 mg of 5-(methanesulfonyloxy)-3-methyl-3-(4-nitrophenyl)pentyl methanesulfonate (34.46%) as a yellow oil. LRMS (ES) m/z 396 (M+H).

### Step 6: Preparation of 4-methyl-4-(4-nitrophenyl)tetrahydro-2H-thiopyran (Intermediate 10-f)

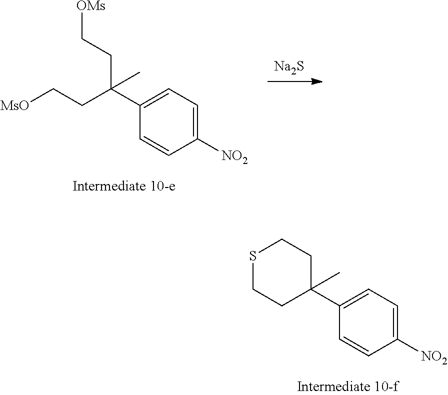 [SMILES: CC(CCOS(C)(=O)=O)(CCOS(C)(=O)=O)c1ccc([N+](=O)[O-])cc1 ; "Na2S" ; CC1(c2ccc([N+](=O)[O-])cc2)CCSCC1]

To a solution of 5-(methanesulfonyloxy)-3-methyl-3-(4-nitrophenyl)pentyl methanesulfonate (410 mg, 1.037 mmol, 1 equiv) in ACN (5 mL) was added Na₂S (49.33 mg, 0.632 mmol, 0.61 equiv). The resulting mixture was stirred at 80° C. for overnight under nitrogen atmosphere, cooled to r.t., added water (20 mL). The mixture was extracted with EtOAc (20 mL) twice. The combined organic layers were washed twice with brine (20 mL), dried over anhydrous Na₂SO₄, concentrated under reduced pressure, purified by silica gel column chromatography, eluted with PE/EtOAc (20:1) to afford 130 mg of 4-methyl-4-(4-nitrophenyl)tetrahydro-2H-thiopyran (52.83%) as a yellow oil. LRMS (ES) m/z 238 (M+H).

### Step 7: Preparation of 4-methyl-4-(4-nitrophenyl)tetrahydro-2H-thiopyran 1,1-dioxide (Intermediate 10-g)

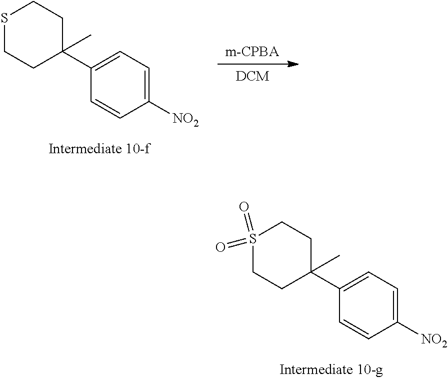

To a solution of 4-methyl-4-(4-nitrophenyl)thiane (130 mg, 0.548 mmol, 1 equiv) in DCM (3 mL) was added m-CPBA (283 mg, 1.640 mmol, 2.99 equiv). The resulting mixture was stirred at r.t. for 2 h, poured into water (10 mL). The aqueous layer was extracted with CH₂Cl₂ (10 mL) twice. The combined organic layers were washed with Na₂S₂O₄ (5 mL) and twice with brine (10 mL), dried over anhydrous Na₂SO₄, concentrated under reduced pressure to afford 170 mg of 4-methyl-4-(4-nitrophenyl)tetrahydro-2H-thiopyran 1,1-dioxide as a yellow solid. LRMS (ES) m/z 270 (M+H).

### Step 8: Preparation of 4-(4-aminophenyl)-4-methyltetrahydro-2H-thiopyran 1,1-dioxide (Intermediate 10.0)

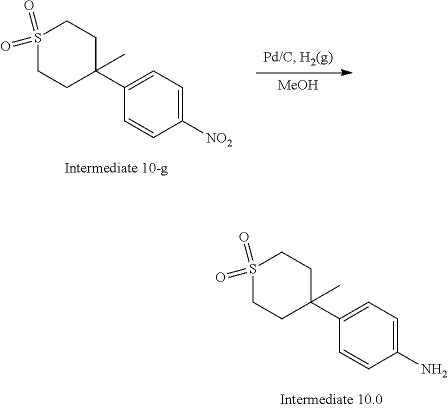 [SMILES: "MeOH" ; CC1(c2ccc([N+](=O)[O-])cc2)CCS(=O)(=O)CC1 ; "Pd/C, H2(g)" ; CC1(c2ccc(N)cc2)CCS(=O)(=O)CC1]

To a solution of 4-methyl-4-(4-nitrophenyl)tetrahydro-2H-thiopyran 1,1-dioxide (170 mg, 0.631 mmol, 1 equiv) in methanol (3 mL) was added Pd/C (85 mg, 50% w/w). The resulting mixture was stirred at r.t. for 1.5 h under hydrogen atmosphere, filtered to remove solids and the filtrate was concentrated under reduced pressure to afford 100 mg of 4-(4-aminophenyl)-4-methyltetrahydro-2H-thiopyran 1,1-dioxide (66.19%) as a brown oil. LRMS (ES) m/z 240 (M+H).

### Example K

### Synthesis of 3-methyl-1-(4-nitrobenzyl)pyrrolidin-2-one (Intermediate 11.1-11.15)

### Preparation of 3-methyl-1-(4-nitrobenzyl)pyrrolidin-2-one (Intermediate 11-a)

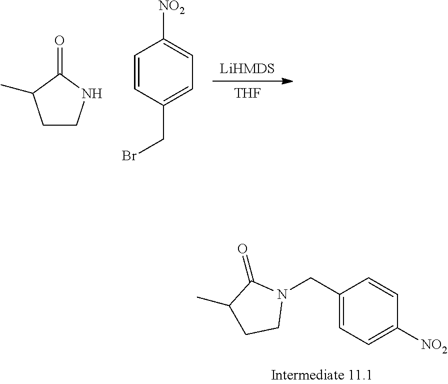

LiHMDS (22.2 mL, 22.2 mmol, 1.1 equiv, 1 M in THF) was added to a stirring solution of 3-methylpyrrolidin-2-one (8.7 g, 20.2 mmol, 1 equiv) in THE (20 mL) at 0° C. After 1 h, benzyl bromide (27 g, 125 mmol, 1.25 equiv) in THE (20 mL) were added and the reaction allowed to return to rt over 12 h. The reaction was dry loaded onto silica and product isolated by silica chromotography (0->100% EtOAc/Hex) as a red tinged solid (24.1 g, 72%). LC/MS (APCI) m/z: 235.1 [M+H]. ¹H NMR (400 MHz, Chloroform-d) δ 8.17 (d, J=8.8 Hz, 2H), 7.38 (d, J=8.4 Hz, 2H), 4.61-4.43 (m, 2H), 3.21 (dd, J=8.2, 5.4 Hz, 2H), 2.55 (t, J=8.1 Hz, 1H), 2.33-2.19 (m, 1H), 1.64 (dq, J=12.2, 8.6 Hz, 1H), 1.23 (d, J=7.1 Hz, 3H).

Intermediate 11.2-11.15 were prepared in a similar manner as Intermediate 11.1

| Intermediate | Reagents | Structure, Name and Data |
|---|---|---|
| 11.2 | tert-butyl 3-oxopiperazine-1- carboxylate | 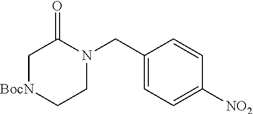 [SMILES: CC(C)(C)OC(=O)N1CCN(Cc2ccc([N+](=O)[O-])cc2)C(=O)C1] |
| | | tert-Butyl 4-(4-nitrobenzyl)-3- |
| | | oxopiperazine-1-carboxylate. LCMS-APCI |
| | | (POS.) m/z: 236.1 (M + H-Boc)⁺. ¹H NMR |
| | | (400 MHz, Chloroform-d) δ 8.17 (d, J = 8.7 |
| | | Hz, 2H), 7.42 (d, J = 8.7 Hz, 2H), 4.69 (s, |
| | | 2H), 4.16 (s, 2H), 3.62 (t, J = 5.4 Hz, 2H), |
| | | 3.29 (t, J = 5.4 Hz, 2H), 1.44 (s, 9H). |
| 11.3 | pyrrolidin-2-one | 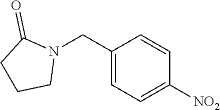 [SMILES: O=C1CCCN1Cc1ccc([N+](=O)[O-])cc1] |
| | | 1-(4-nitrobenzyl)pyrrolidin-2-one. LCMS- |
| | | APCI (POS.) m/z: 221 (M + H)⁺. |
| 11.4 | tert-butyl 3-oxopiperazine-1- carboxylate | 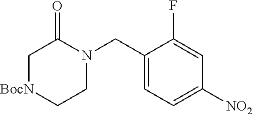 [SMILES: CC(C)(C)OC(=O)N1CCN(Cc2ccc([N+](=O)[O-])cc2F)C(=O)C1] |
| | | tert-butyl 4-(2-fluoro-4-nitrobenzyl)-3- |
| | | oxopiperazine-1-carboxylate. LCMS-APCI |
| | | (POS.) m/z: 354 (M + H)⁺. |
| 11.4 | 4-methylpiperazin-2-one | 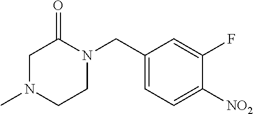 [SMILES: CN1CCN(Cc2ccc([N+](=O)[O-])c(F)c2)C(=O)C1] |
| | | 1-(3-fluoro-4-nitrobenzyl)-4- |
| | | methylpiperazin-2-one. LCMS-APCI |
| | | (POS.) m/z: 268 (M + H)⁺. |
| 11.5 | tert-butyl 2-methyl-5- oxopiperazine-1-carboxylate | 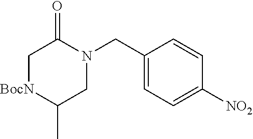 [SMILES: CC1CN(Cc2ccc([N+](=O)[O-])cc2)C(=O)CN1C(=O)OC(C)(C)C] |
| | | tert-butyl 2-methyl-4-(4-nitrobenzyl)-5- |
| | | oxopiperazine-1-carboxylate. LCMS-APCI |
| | | (POS.) m/z: 350 (M + H)⁺. |
| 11.6 | tert-butyl 3-methyl-5- oxopiperazine-1-carboxylate | 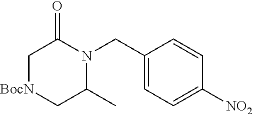 [SMILES: CC1CN(C(=O)OC(C)(C)C)CC(=O)N1Cc1ccc([N+](=O)[O-])cc1] |
| | | tert-butyl 3-methyl-4-(4-nitrobenzyl)-5- |
| | | oxopiperazine-1-carboxylate. LCMS-APCI |
| | | (POS.) m/z: 294 (M + H-Boc)⁺. |
| 11.7 | piperidin-2-one | 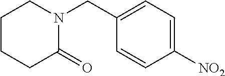 [SMILES: O=C1CCCCN1Cc1ccc([N+](=O)[O-])cc1] |
| | | 1-(4-nitrobenzyl)piperidin-2-one. LCMS- |
| | | APCI (POS.) m/z: 235 (M + H)⁺. |
| 11.8 | tert-butyl 2-methyl-3- oxopiperazine-1-carboxylate | 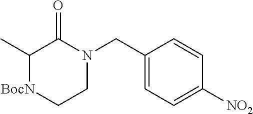 [SMILES: CC1C(=O)N(Cc2ccc([N+](=O)[O-])cc2)CCN1C(=O)OC(C)(C)C] |
| | | tert-butyl 2-methyl-4-(4-nitrobenzyl)-3- |
| | | oxopiperazine-1-carboxylate. LCMS-APCI |
| | | (POS.) m/z: 350 (M + H)⁺. |
| 11.9 | 5-methylpiperidin-2-one | 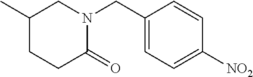 [SMILES: CC1CCC(=O)N(Cc2ccc([N+](=O)[O-])cc2)C1] |
| | | 5-methyl-1-(4-nitrobenzyl)piperidin-2-one. |
| | | LCMS-APCI (POS.) m/z: 249 (M + H)⁺. |
| 11.10 | 4-methylpiperidin-2-one | 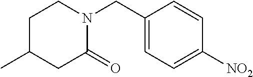 [SMILES: CC1CCN(Cc2ccc([N+](=O)[O-])cc2)C(=O)C1] |
| | | 4-methyl-1-(4-nitrobenzyl)piperidin-2-one. |
| | | LCMS-APCI (POS.) m/z: 249 (M + H)⁺. |
| 11.11 | 3-methylpiperidin-2-one | 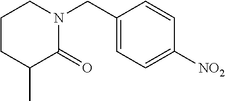 [SMILES: CC1CCCN(Cc2ccc([N+](=O)[O-])cc2)C1=O] |
| | | 3-methyl-1-(4-nitrobenzyl)piperidin-2-one. |
| | | LCMS-APCI (POS.) m/z: 249 (M + H)⁺. |
| 11.12 | 3-(methylsulfonyl)azetidine | 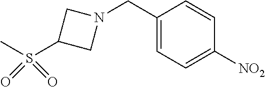 [SMILES: CS(=O)(=O)C1CN(Cc2ccc([N+](=O)[O-])cc2)C1] |
| | | 3-(methylsulfonyl)-1-(4- |
| | | nitrobenzyl)azetidine. LCMS-APCI (POS.) |
| | | m/z: 271 (M + H)⁺. ¹H NMR (400 MHz, |
| | | DMSO-d₆) δ 8.18 (d, J = 8.7 Hz, 2H), 7.56 |
| | | (d, J = 8.7 Hz, 2H), 4.19 (dd, J = 8.1, 6.1, |
| | | 1.9 Hz, 1H), 3.77 (s, 2H), 3.56 (d, J = 8.1 |
| | | Hz, 2H), 3.51-3.41 (m, 2H), 2.98 (s, 3H). |
| 11.13 | 2-oxa-7-azaspiro[3.5]nonan-6- one | 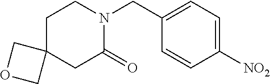 [SMILES: O=C1CC2(CCN1Cc1ccc([N+](=O)[O-])cc1)COC2] |
| | | 7-(4-nitrobenzyl)-2-oxa-7- |
| | | azaspiro[3.5]nonan-6-one. LC/MS (APCI) |
| | | m/z: 277.1 [M + H]. |
| 11.14 | 3-azabicyclo[3.1.0]hexan-2-one | 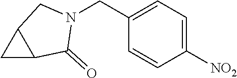 [SMILES: O=C1C2CC2CN1Cc1ccc([N+](=O)[O-])cc1] |
| | | 3-(4-nitrobenzyl)-3-azabicyclo[3.1.0]hexan- |
| | | 2-one. LC/MS (APCI) m/z: 233.0 [M + H]. |
| 11.15 | 2-azabicyclo[3.1.0]hexan-3-one | 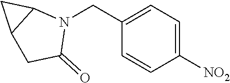 [SMILES: O=C1CC2CC2N1Cc1ccc([N+](=O)[O-])cc1] |
| | | 2-(4-nitrobenzyl)-2-azabicyclo[3.1.0]hexan- |
| | | 3-one. LC/MS (APCI) m/z: 233.1 [M + H]. |

### Example L

### Synthesis of 5-methyl-1-(4-nitrobenzyl)pyrrolidin-2-one (Intermediate 12.1-12.2)

### Preparation of 5-methyl-1-(4-nitrobenzyl)pyrrolidin-2-one (Intermediate 12)

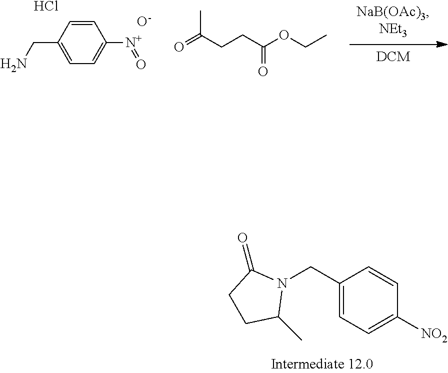

Sodium triacetoxyborohydride (11 g, 53 mmol, 2 equiv) was added to a stirring solution of (4-nitrophenyl)methanamine hydrochloride (5 g, 26.5 mmol, 1 equiv), ethyl 4-oxopentanoate (4.2 g, 29.2 mmol, 1.1 equiv), and triethylamine (3.6 mL, 26.5 mmol, 1 equiv) in DCM (200 mL) at rt. After 14 h, the reaction was dry loaded onto silica and product isolated by silica chromatography as a white solid (5 g, 81%). LC/MS (APCI) m/z: 235.1 [M+H]. ¹H NMR (400 MHz, Chloroform-d) δ 8.20 (d, J=8.3 Hz, 2H), 7.43 (d, J=8.3 Hz, 2H), 4.90 (d, J=15.6 Hz, 1H), 4.25 (d, J=15.6 Hz, 1H), 3.58 (h, J=6.3 Hz, 1H), 2.50 (dtd, J=34.1, 17.1, 9.5 Hz, 2H), 2.23 (ddd, J=13.3, 11.0, 6.8 Hz, 1H), 1.67 (ddt, J=13.2, 9.3, 6.8 Hz, 1H), 1.18 (d, J=6.2 Hz, 3H).

Intermediate 12.2 was prepared in a similar manner as Intermediate 12.1

| Intermediate | Reagents | Structure, Name and Data |
|---|---|---|
| 12.2 | ethyl 5- oxohexanoate | 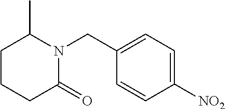 [SMILES: CC1CCCC(=O)N1Cc1ccc([N+](=O)[O-])cc1] |
| | | 6-methyl-1-(4-nitrobenzyl)piperidin-2-one. |
| | | LC/MS (APCI) m/z: 249 [M + H]. |

### Example M

### Synthesis of 1-(4-aminobenzyl)-3-methylpyrrolidin-2-one (Intermediate 13.1-13.X)

### Preparation of 1-(4-aminobenzyl)-3-methylpyrrolidin-2-one (Intermediate 13.1)

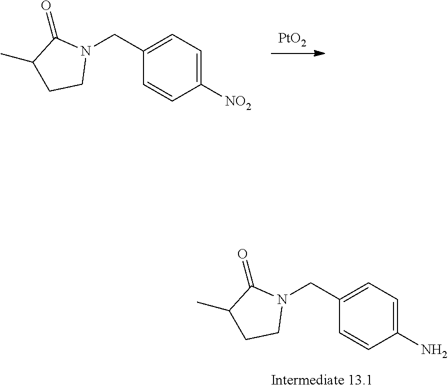 [SMILES: CC1CCN(Cc2ccc([N+](=O)[O-])cc2)C1=O ; "PtO2" ; CC1CCN(Cc2ccc(N)cc2)C1=O]

3-Methyl-1-(4-nitrobenzyl)pyrrolidin-2-one (3 g, 12.8 mmol, 1 equiv) and PtO₂ (0.29 g, 1.28 mmol, 0.1 equiv) were stirred under H₂ (80 psi) for 1 h. The reaction was filtered through a pad of celite, solvent removed by rotary evaporated, and dried under high vacuum to give the product as a red tinged solid (2.6 g, 99%). LC/MS (APCI) m/z: 205.2 [M+H].

Intermediates 13.2-13.36 were prepared in a similar manner as Intermediate 13.1

| Intermediate | Reagents | Structure, Name and Data |
|---|---|---|
| 13.2 | Intermediate 11 | 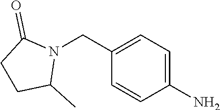 |
| | | 1-(4-aminobenzyl)-5-methylpyrrolidin-2- |
| | | one. LC/MS (APCI) m/z: 205.1 [M + H]. |
| 13.3 | Intermediate 14 | 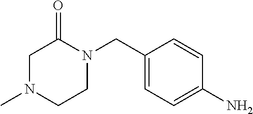 [SMILES: CN1CCN(Cc2ccc(N)cc2)C(=O)C1] |
| | | 4-Methyl-1-(4-nitrobenzyl)piperazin-2-one. |
| | | LC/MS (APCI) m/z: 220.2 [M + H]. ¹H |
| | | NMR (400 MHz, Methanol-d₄) δ 7.04 (d, |
| | | J = 8.1 Hz, 2H), 6.70 (d, J = 8.3 Hz, 2H), 4.48 |
| | | (s, 2H), 3.29 (t, J = 5.6 Hz, 2H), 3.15 (s, |
| | | 2H), 2.66 (t, J = 5.6 Hz, 2H), 2.33 (s, 3H). |
| 13.4 | Intermediate 11.2 | 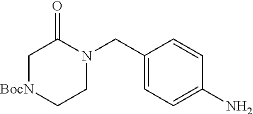 [SMILES: CC(C)(C)OC(=O)N1CCN(Cc2ccc(N)cc2)C(=O)C1] |
| | | tert-butyl 4-(4-aminobenzyl)-3- |
| | | oxopiperazine-1-carboxylate. LC/MS |
| | | (APCI) m/z: 250 [M + H]. |
| 13.5 | Intermediate 11.3 | 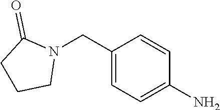 [SMILES: Nc1ccc(CN2CCCC2=O)cc1] |
| | | 1-(4-aminobenzyl)pyrrolidin-2-one. LC/MS |
| | | (APCI) m/z: 191 [M + H]. |
| 13.6 | Intermediate 21 | 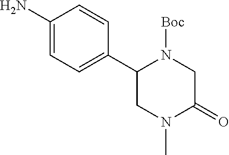 [SMILES: CN1CC(c2ccc(N)cc2)N(C(=O)OC(C)(C)C)CC1=O] |
| | | tert-butyl 4-methyl-2-(4-nitrophenyl)-5- |
| | | oxopiperazine-1-carboxylate. LC/MS |
| | | (APCI) m/z: 306 [M + H]. |
| 13.7 | Intermediate 22 | 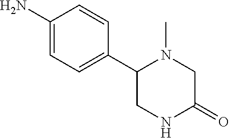 |
| | | 5-(4-aminophenyl)-4-methylpiperazin-2- |
| | | one. LC/MS (APCI) m/z: 206 [M + H]. |
| 13.8 | Intermediate 23 | 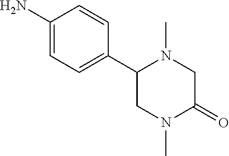 [SMILES: CN1CC(c2ccc(N)cc2)N(C)CC1=O] |
| | | 5-(4-aminophenyl)-1,4-dimethylpiperazin-2- |
| | | one. LC/MS (APCI) m/z: 220 [M + H]. |
| 13.9 | Intermediate 24 | 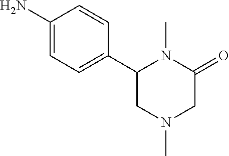 [SMILES: CN1CC(=O)N(C)C(c2ccc(N)cc2)C1] |
| | | 6-(4-aminophenyl)-1,4-dimethylpiperazin-2- |
| | | one LC/MS (APCI) m/z: 220 [M + H]. |
| 13.10 | Intermediate 25.1 | 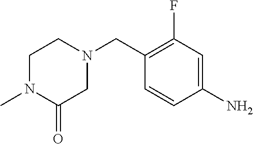 [SMILES: CN1CCN(Cc2ccc(N)cc2F)CC1=O] |
| | | 4-(4-amino-2-fluorobenzyl)-1- |
| | | methylpiperazin-2-one. LC/MS (APCI) |
| | | m/z: 238 [M + H]. |
| 13.11 | Intermediate 25.2 | 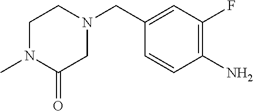 [SMILES: CN1CCN(Cc2ccc(N)c(F)c2)CC1=O] |
| | | 4-(4-amino-3-fluorobenzyl)-1- |
| | | methylpiperazin-2-one. LC/MS (APCI) |
| | | m/z: 238 [M + H]. |
| 13.12 | Intermediate 14.2 | 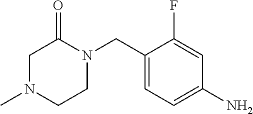 [SMILES: CN1CCN(Cc2ccc(N)cc2F)C(=O)C1] |
| | | 1-(4-amino-2-fluorobenzyl)-4- |
| | | methylpiperazin-2-one. LC/MS (APCI) |
| | | m/z: 238 [M + H]. |
| 13.13 | Intermediate 11.4 | 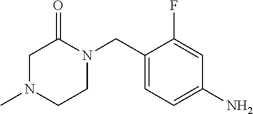 [SMILES: CN1CCN(Cc2ccc(N)cc2F)C(=O)C1] |
| | | 1-(4-amino-2-fluorobenzyl)-4- |
| | | methylpiperazin-2-one. LC/MS (APCI) |
| | | m/z: 238 [M + H]. |
| 13.14 | Intermediate 14.3 | 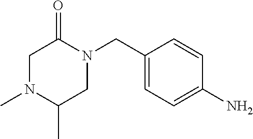 [SMILES: CC1CN(Cc2ccc(N)cc2)C(=O)CN1C] |
| | | 1-(4-aminobenzyl)-4,5-dimethylpiperazin-2- |
| | | one. LC/MS (APCI) m/z: 234 [M + H]. |
| 13.15 | Intermediate 14.4 | 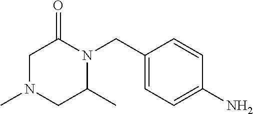 [SMILES: CC1CN(C)CC(=O)N1Cc1ccc(N)cc1] |
| | | 1-(4-aminobenzyl)-4,6-dimethylpiperazin-2- |
| | | one. LC/MS (APCI) m/z: 234 [M + H]. |
| 13.16 | Intermediate 11.5 | 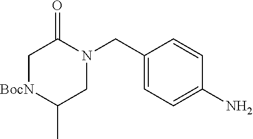 [SMILES: CC1CN(Cc2ccc(N)cc2)C(=O)CN1C(=O)OC(C)(C)C] |
| | | tert-butyl 4-(4-aminobenzyl)-2-methyl-5- |
| | | oxopiperazine-1-carboxylate. LCMS-APCI |
| | | (POS.) m/z: 320 (M + H)⁺. |
| 13.17 | Intermediate 11.6 | 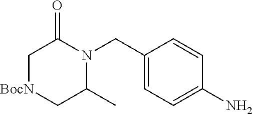 [SMILES: CC1CN(C(=O)OC(C)(C)C)CC(=O)N1Cc1ccc(N)cc1] |
| | | tert-butyl 4-(4-aminobenzyl)-3-methyl-5- |
| | | oxopiperazine-1-carboxylate. LCMS-APCI |
| | | (POS.) m/z: 264 (M + H-56)⁺. |
| 13.18 | Intermediate 26 | 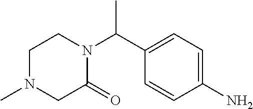 [SMILES: CC(c1ccc(N)cc1)N1CCN(C)CC1=O] |
| | | 1-(1-(4-aminophenyl)ethyl)-4- |
| | | methylpiperazin-2-one. LCMS-APCI |
| | | (POS.) m/z: 234 (M + H)⁺. |
| 13.19 | Intermediate 14.4 | 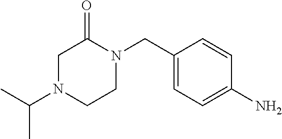 [SMILES: CC(C)N1CCN(Cc2ccc(N)cc2)C(=O)C1] |
| | | 1-(4-aminobenzyl)-4-isopropylpiperazin-2- |
| | | one. LC/MS (APCI) m/z: 248 [M + H]. |
| 13.20 | Intermediate 28 | 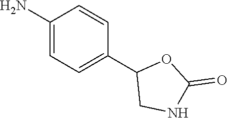 [SMILES: Nc1ccc(C2CNC(=O)O2)cc1] |
| | | 5-(4-aminophenyl)oxazolidin-2-one. LC/MS |
| | | (APCI) m/z: 179 [M + H]. |
| 13.21 | Intermediate 29 | 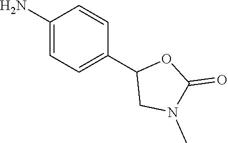 |
| | | 5-(4-aminophenyl)-3-methyloxazolidin-2- |
| | | one. LC/MS (APCI) m/z: 193 [M + H]. |
| 13.22 | Intermediate 30.1 | 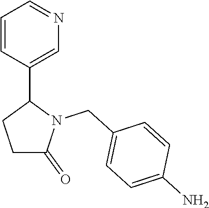 [SMILES: Nc1ccc(CN2C(=O)CCC2c2cccnc2)cc1] |
| | | 1-(4-aminobenzyl)-5-(pyridin-3- |
| | | yl)pyrrolidin-2-one. LC/MS (APCI) m/z: |
| | | 268 [M + H]. |
| 13.23 | Intermediate 30.2 | 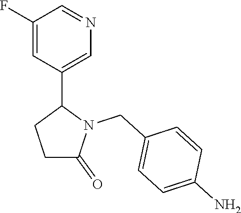 [SMILES: Nc1ccc(CN2C(=O)CCC2c2cncc(F)c2)cc1] |
| | | 1-(4-aminobenzyl)-5-(5-fluoropyridin-3- |
| | | yl)pyrrolidin-2-one. LCMS-APCI (POS.) |
| | | m/z: 286 (M + H)⁺. |
| 13.24 | Intermediate 11.7 | 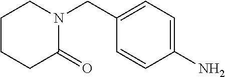 [SMILES: Nc1ccc(CN2CCCCC2=O)cc1] |
| | | 1-(4-aminobenzyl)piperidin-2-one. LCMS- |
| | | APCI (POS.) m/z: 205 (M + H)⁺. |
| 13.25 | Intermediate 14.6 | 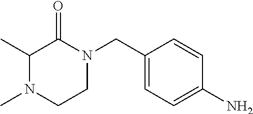 [SMILES: CC1C(=O)N(Cc2ccc(N)cc2)CCN1C] |
| | | 1-(4-aminobenzyl)-3,4-dimethylpiperazin-2- |
| | | one. LC/MS (APCI) m/z: 234 [M + H]. |
| 13.26 | Intermediate 31 | 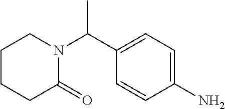 [SMILES: CC(c1ccc(N)cc1)N1CCCCC1=O] |
| | | 1-(1-(4-aminophenyl)ethyl)piperidin-2-one. |
| | | LC/MS (APCI) m/z: 219 [M + H]. |
| 13.27 | Intermediate 12.2 | 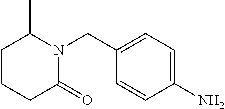 [SMILES: CC1CCCC(=O)N1Cc1ccc(N)cc1] |
| | | 1-(4-aminobenzyl)-6-methylpiperidin-2-one. |
| | | LC/MS (APCI) m/z: 218 [M + H]. |
| 13.28 | Intermediate 11.9 | 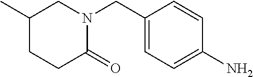 [SMILES: CC1CCC(=O)N(Cc2ccc(N)cc2)C1] |
| | | 1-(4-aminobenzyl)-5-methylpiperidin-2-one. |
| | | LCMS-APCI (POS.) m/z: 219 (M + H)⁺. |
| 13.29 | Intermediate 11.10 | 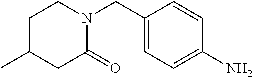 [SMILES: CC1CCN(Cc2ccc(N)cc2)C(=O)C1] |
| | | 1-(4-aminobenzyl)-4-methylpiperidin-2-one. |
| | | LCMS-APCI (POS.) m/z: 219 (M + H)⁺. |
| 13.30 | Intermediate 11.11 | 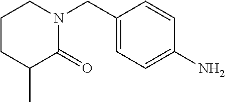 [SMILES: CC1CCCN(Cc2ccc(N)cc2)C1=O] |
| | | 1-(4-aminobenzyl)-3-methylpiperidin-2-one. |
| | | LCMS-APCI (POS.) m/z: 219 (M + H)⁺. |
| 13.31 | Intermediate 34.1 | 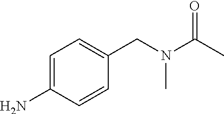 |
| | | N-(4-aminobenzyl)-N-methylacetamide. |
| | | LCMS-APCI (POS.) m/z: 178 (M + H)⁺. |
| 13.32 | Intermediate 34.2 | 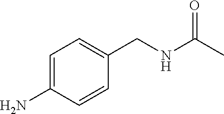 |
| | | N-(4-aminobenzyl)acetamide. LCMS-APCI |
| | | (POS.) m/z: 164 (M + H)⁺. |
| 13.33 | Intermediate 11.12 | 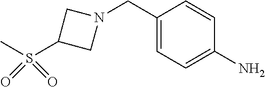 [SMILES: CS(=O)(=O)C1CN(Cc2ccc(N)cc2)C1] |
| | | 4-((3-(methylsulfonyl)azetidin-1- |
| | | yl)methyl)aniline. LCMS-APCI (POS.) |
| | | m/z: 241.1 (M + H)⁺. |
| 13.34 | Intermediate 11.13 | 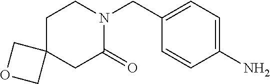 [SMILES: Nc1ccc(CN2CCC3(COC3)CC2=O)cc1] |
| | | 7-(4-aminobenzyl)-2-oxa-7- |
| | | azaspiro[3.5]nonan-6-one. LC/MS (APCI) |
| | | m/z: 247.1 [M + H]. |
| 13.35 | Intermediate 11.14 | 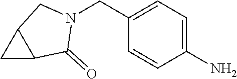 [SMILES: Nc1ccc(CN2CC3CC3C2=O)cc1] |
| | | 3-(4-aminobenzyl)-3- |
| | | azabicyclo[3.1.0]hexan-2-one. LC/MS |
| | | (APCI) m/z: 203.1 [M + H]. |
| 13.36 | Intermediate 11.15 | 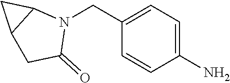 [SMILES: Nc1ccc(CN2C(=O)CC3CC32)cc1] |
| | | 2-(4-aminobenzyl)-2- |
| | | azabicyclo[3.1.0]hexan-3-one. LC/MS |
| | | (APCI) m/z: 203.1 [M + H]. |

### Example N

### Synthesis of 4-Methyl-1-(4-nitrobenzyl)piperazin-2-one (Intermediate 14.1-14.6)

### Step 1: Preparation of 1-(4-nitrobenzyl)piperazin-2-one hydrochloride (Intermediate 14-a)

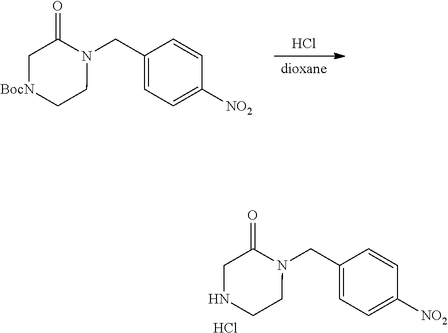

tert-Butyl 4-(4-nitrobenzyl)-3-oxopiperazine-1-carboxylate (Intermediate 11.2, 24.1 g, 71.9 mmol, 1 equiv) was suspended 4M HCl in dioxanes (180 mL, 719 mmol, 10 equiv) at rt. After 2 h, the solvent was removed by rotary evaporation and dried under high vacuum to give the desired product as a white solid (19.5 g, 99.9%). LCMS-APCI (POS.) m/z: 236.1 (M+H)⁺.

### Step 2: Preparation of 4-Methyl-1-(4-nitrobenzyl)piperazin-2-one (Intermediate 14.1)

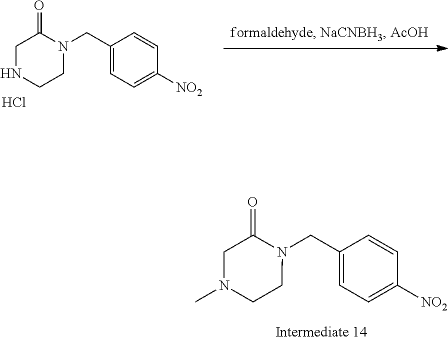 [SMILES: O=C1CNCCN1Cc1ccc([N+](=O)[O-])cc1 ; "formaldehyde, NaCNBH3, AcOH" ; Cl ; CN1CCN(Cc2ccc([N+](=O)[O-])cc2)C(=O)C1]

Formaldehyde (17.47 g, 215.3 mmol, 3 equiv, 37% in water) and AcOH (12.9 mL, 215.3 mmol, 3 equiv) were added to a stirring suspension of 1-(4-nitrobenzyl)piperazin-2-one hydrochloride (19.5 g, 71.8 mmol, 1 equiv) in MeOH (800 mL) at rt. After 10 min the reaction became homogenous and was subsequently cooled to 0° C. before NaCNBH₃ (9.9 g, 157.9 mmol, 2.2 equiv) was added and the reaction warmed to rt. After 3 h, the total volume was reduced to −400 mL by rotary evaporation, quenched with saturated sodium bicarbonate (1 L), extracted with DCM (3×750 mL), organics combined, dried over sodium sulfate, filtered, and solvent removed by rotary evaporation. The oily yellow product then crystalized overnight under high vacuum to give the product as pale yellow crystals (17 g, 95%). LCMS-APCI (POS.) m/z: 250.1 (M+H)⁺. ¹H NMR (400 MHz, Chloroform-d) δ 8.11 (d, J=8.7 Hz, 2H), 7.35 (d, J=8.7 Hz, 2H), 4.62 (s, 2H), 3.25-3.18 (m, 2H), 3.14 (s, 2H), 2.63-2.53 (m, 2H), 2.28 (s, 3H).

Intermediates 14.2-14.6 was prepared in a similar manner as Intermediate 14.1

| Intermediate | Reagents | Structure, Name and Data |
|---|---|---|
| 14.2 | Intermediate 11.4 | 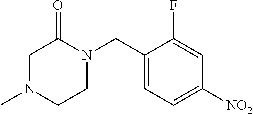 [SMILES: CN1CCN(Cc2ccc([N+](=O)[O-])cc2F)C(=O)C1] |
| | | 1-(2-fluoro-4-nitrobenzyl)-4- |
| | | methylpiperazin-2-one. LC/MS (APCI) |
| | | m/z: 268 [M + H]. |
| 14.3 | Intermediate 11.5 | 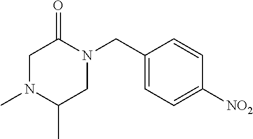 [SMILES: CC1CN(Cc2ccc([N+](=O)[O-])cc2)C(=O)CN1C] |
| | | 4,5-dimethyl-1-(4-nitrobenzyl)piperazin-2- |
| | | one. LC/MS (APCI) m/z: 264 [M + H]. |
| 14.4 | Intermediate 11.6 | 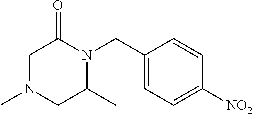 [SMILES: CC1CN(C)CC(=O)N1Cc1ccc([N+](=O)[O-])cc1] |
| | | 4,6-dimethyl-1-(4-nitrobenzyl)piperazin-2- |
| | | one. LC/MS (APCI) m/z: 264 [M + H]. |
| 14.5 | Intermediate 11.2 | 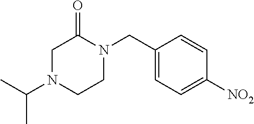 [SMILES: CC(C)N1CCN(Cc2ccc([N+](=O)[O-])cc2)C(=O)C1] |
| | | 4-isopropyl-1-(4-nitrobenzyl)piperazin-2- |
| | | one. LC/MS (APCI) m/z: 278 [M + H]. |
| 14.6 | Intermediate 11.8 | 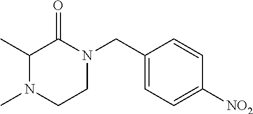 [SMILES: CC1C(=O)N(Cc2ccc([N+](=O)[O-])cc2)CCN1C] |
| | | 3,4-dimethyl-1-(4-nitrobenzyl)piperazin-2- |
| | | one. LC/MS (APCI) m/z: 264 [M + H]. |

### Example 0

### Synthesis of 4-((azetidin-1-ylsulfonyl)methyl)aniline (Intermediate 15.1-15.4)

### Step 1: Preparation of 1-((4-nitrobenzyl)sulfonyl)azetidine (Intermediate 15-a)

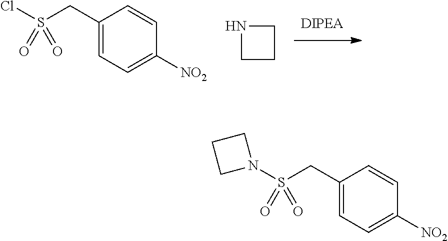

(4-Nitrophenyl)methanesulfonyl chloride (500 mg, 2.12 mmol, 1 equiv) was added to a stirring solution of azetadine (121 mg, 2.12 mmol, 1 equiv) and diisoproylethylamine (1.1 mL, 6.4 mmol, 3 equiv) in DCM (5 mL) at rt. After 1 h, the reaction was washed with saturated sodium bicarbonate (5 mL), dried over sodium sulfate, filtered, and solvent removed by rotary evaporation. The crude material was resolved by silica chromatography (0->3% MeOH/DCM) to give 1-((4-nitrobenzyl)sulfonyl)azetidine (110 mg, 20%). LCMS-APCI (Neg.) m/z: 255.2 (M−H). ¹H NMR (400 MHz, DMSO-d₆) δ 8.27 (d, J=8.8 Hz, 1H), 7.72 (d, J=8.8 Hz, 1H), 4.73 (s, 1H), 3.89 (t, J=7.7 Hz, 2H), 2.19 (p, J=7.7 Hz, 1H).

### Step 2: Preparation of 4-((azetidin-1-ylsulfonyl)methyl)aniline (Intermediate 15.1)

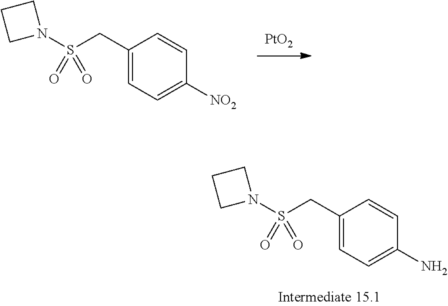 [SMILES: O=[N+]([O-])c1ccc(CS(=O)(=O)N2CCC2)cc1 ; "PtO2" ; Nc1ccc(CS(=O)(=O)N2CCC2)cc1]

1-((4-nitrobenzyl)sulfonyl)azetidine (110 mg, 0.43 mmol, 1 equiv) and PtO2 (5 mg, 0.022 mmol, 0.05 equiv) were suspended in MeOH (5 mL) before being stirred under H2 for 12 h. The reaction was filtered through a 0.45 μm PTFE syringe filter and solvent removed by rotary evaporation to give the product (90 mg, 93%). LCMS-APCI (POS.) m/z: 227.2 (M+H)⁺.

Intermediates 15.2-15.4 were prepared in a similar manner as Intermediate 15.1

| Intermediate | Reagents | Structure, Name and Data |
|---|---|---|
| 15.2 | pyrrolidine | 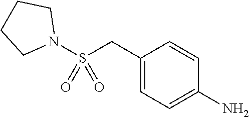 [SMILES: Nc1ccc(CS(=O)(=O)N2CCCC2)cc1] |
| | | 4-((pyrrolidin-1-ylsulfonyl)methyl)aniline. |
| | | LC/MS (APCI) m/z: 241.2 [M + H]. |
| 15.3 | piperidine | 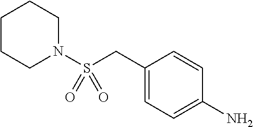 [SMILES: Nc1ccc(CS(=O)(=O)N2CCCCC2)cc1] |
| | | 4-((piperidin-1-ylsulfonyl)methyl)aniline. |
| | | LC/MS (APCI) m/z: 255.2 [M + H]. |
| 15.4 | aniline | 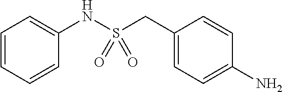 [SMILES: Nc1ccc(CS(=O)(=O)Nc2ccccc2)cc1] |
| | | 1-(4-aminophenyl)-N- |
| | | phenylmethanesulfonamide. LC/MS (APCI) |
| | | m/z: 263.2 [M + H]. |

### Example P

### Synthesis of 1-(4-chlorobenzyl)-3-(4-formylphenyl)urea (Intermediate 16)

### Step 1: Preparation of phenyl (4-chlorobenzyl)carbamate (Intermediate 16-a)

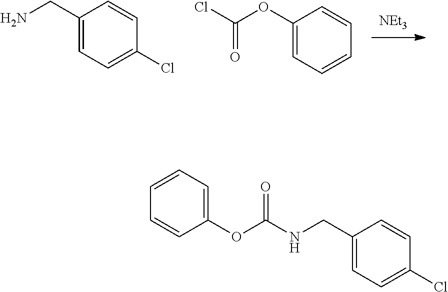 [SMILES: NCc1ccc(Cl)cc1 ; O=C(Cl)Oc1ccccc1 ; O=C(NCc1ccc(Cl)cc1)Oc1ccccc1 ; "NEt3"]

To a stirred solution of 1-(4-chlorophenyl)methanamine (10.00 g, 70.621 mmol, 1 equiv) and NEt₃ (10.72 g, 105.9 mmol, 1.5 equiv) in THE (100 mL) at 0° C. was added phenyl chloroformate (12.16 g, 77.6 mmol, 1.1 equiv) dropwise over a period of 15 min. The resulting mixture was stirred at r.t. for 3 h, concentrated under reduced pressure, purified by silica gel column chromatography, eluted with PE/EtOAc (4:1) to afford 17.76 g (91.38%) of phenyl (4-chlorobenzyl)carbamate as a pink solid. LCMS-APCI (POS.) m/z: 362 (M+H)⁺.

### Step 2: Preparation of 1-(4-chlorobenzyl)-3-(4-formylphenyl)urea (Intermediate 16-b)

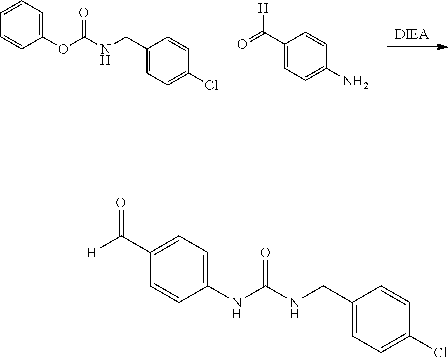 [SMILES: O=C(NCc1ccc(Cl)cc1)Oc1ccccc1 ; Nc1ccc(C=O)cc1 ; "DIEA" ; O=Cc1ccc(NC(=O)NCc2ccc(Cl)cc2)cc1]

To a stirred solution of phenyl (4-chlorobenzyl)carbamate (7.80 g, 29.8 mmol, 1.2 equiv) and 4-aminobenzaldehyde (3.00 g, 24.8 mmol, 1 equiv) in i-PrOH (30.00 mL) were added diisopropylethylamine (16.00 g, 123.8 mmol, 5 equiv). The resulting mixture was stirred at 90° C. for overnight, cooled down to r.t., added water (100 mL) and extracted twice with EtOAc (100 mL). The combined organic layers were washed twice with brine (100 mL), dried over anhydrous Na₂SO₄, concentrated under reduced pressure, purified by silica gel column chromatography, eluted with PE/EtOAc (2:1) to afford 2.04 g (27%) of 1-(4-chlorobenzyl)-3-(4-formylphenyl)urea as a yellow solid. LCMS-APCI (POS.) m/z: 289 (M+H)⁺.

### Step 3: Preparation of 1-(4-chlorobenzyl)-3-(4-(hydroxymethyl)phenyl)urea (Intermediate 16-c)

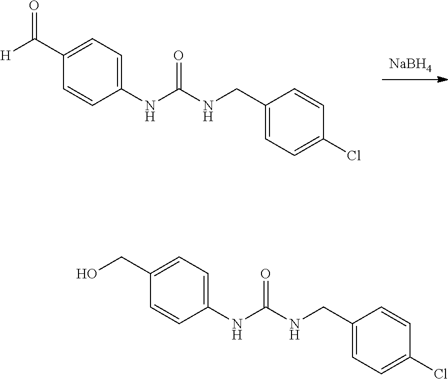 [SMILES: O=Cc1ccc(NC(=O)NCc2ccc(Cl)cc2)cc1 ; "NaBH4" ; O=C(NCc1ccc(Cl)cc1)Nc1ccc(CO)cc1]

To a stirred solution of 1-(4-chlorobenzyl)-3-(4-formylphenyl)urea (2 g, 6.9 mmol, 1 equiv) in EtOH (40 mL) at 0° C. was added NaBH₄ (390 mg, 10.4 mmol, 1.5 equiv). The resulting mixture was stirred at r.t. for 2 h, quenched by the addition of water (50 mL) at 0° C., and extracted twice with EtOAc (50 mL). The combined organic layers were washed twice with water (50 mL), dried over anhydrous Na₂SO₄, concentrated under reduced pressure to afford 2.08 g of 1-(4-chlorobenzyl)-3-(4-(hydroxymethyl)phenyl)urea as a yellow solid. LCMS-APCI (POS.) m/z: 291 (M+H)⁺.

### Step 4: Preparation of 1-(4-chlorobenzyl)-3-(4-(hydroxymethyl)phenyl)urea (Intermediate 16)

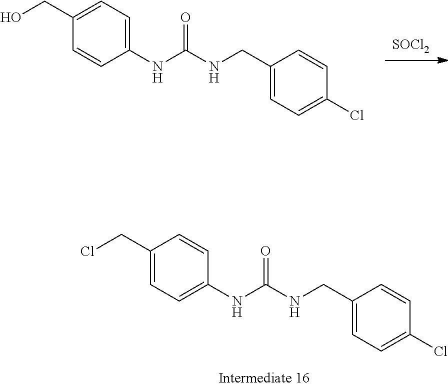 [SMILES: O=C(NCc1ccc(Cl)cc1)Nc1ccc(CO)cc1 ; "SOCl2" ; O=C(NCc1ccc(Cl)cc1)Nc1ccc(CCl)cc1]

To a stirred solution of 1-(4-chlorobenzyl)-3-(4-(hydroxymethyl)phenyl)urea (2 g, 6.9 mmol, 1 equiv) in DCM (20 mL) at 0° C. was added SOCl₂ (1.65 g, 13.9 mmol, 2 equiv). The resulting mixture was stirred at r.t. for 2 h, concentrated under reduced pressure to afford 2.2 g of 1-[4-(chloromethyl)phenyl]-3-[(4-chlorophenyl)methyl]urea as a brown solid. LCMS-APCI (POS.) m/z: 309 (M+H)⁺.

### Example Q

### Synthesis of 1-(4-chlorobenzyl)-3-(4-(((1,1-dioxidotetrahydrothiophen-3-yl)(methyl)amino)methyl)phenyl)urea (Intermediate 17.1-17.6)

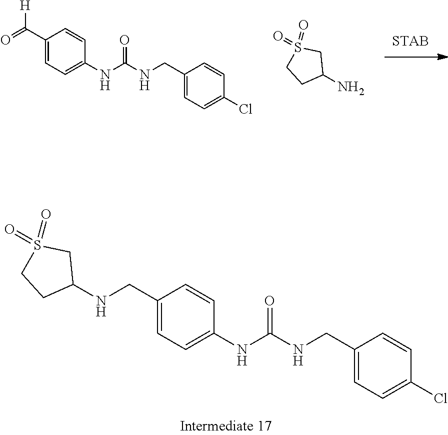 [SMILES: O=Cc1ccc(NC(=O)NCc2ccc(Cl)cc2)cc1 ; NC1CCS(=O)(=O)C1 ; "STAB" ; O=C(NCc1ccc(Cl)cc1)Nc1ccc(CNC2CCS(=O)(=O)C2)cc1]

To a stirred mixture of 3-[(4-chlorophenyl)methyl]-1-(4-formylphenyl)urea (Intermediate 3.2, 300.00 mg, 1.039 mmol, 1.00 equiv) and 3-aminotetrahydrothiophene 1,1-dioxide (168.55 mg, 1.247 mmol, 1.2 equiv) in DCE (10 mL) at 0° C. was added STAB (440.43 mg, 2.078 mmol, 2 equiv). The resulting mixture was stirred at r.t. for overnight, concentrated under reduced pressure, and purified by C18 column chromatography, eluted with water(0.05% NH4HCO3): ACN (2:1) to afford 240 mg of 1-(4-chlorobenzyl)-3-(4-(((1,1-dioxidotetrahydrothiophen-3-yl)amino)methyl)phenyl)urea (56.63) as a white solid. LCMS-APCI (PO3.) m/z: 408 (M+H)⁺.

Intermediates 17.2-17.6 were prepared in a similar manner as Intermediate 17.1

| Intermediate | Aldehyde | Amine | Structure, Name and Data |
|---|---|---|---|
| 17.2 | Intermediate 3.2 | 3- amino pyrrolidin- 2-one | 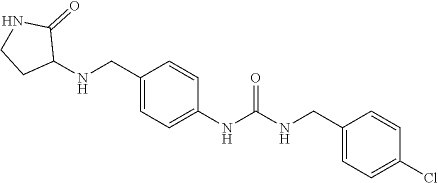 [SMILES: O=C(NCc1ccc(Cl)cc1)Nc1ccc(CNC2CCNC2=O)cc1] |
| | | | 1-(4-chlorobenzyl)-3-(4-(((2-oxopyrrolidin-3- |
| | | | yl)amino)methyl)phenyl)urea. LCMS-APCI (POS.) m/z: |
| | | | 373 (M + H)⁺. |
| 17.3 | Intermediate 3.2 | 3- amino- 1- methyl pyrrolidin- 2-one | 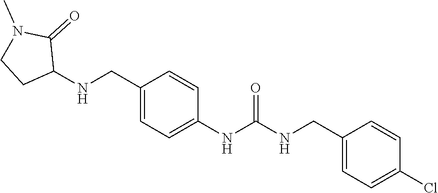 [SMILES: CN1CCC(NCc2ccc(NC(=O)NCc3ccc(Cl)cc3)cc2)C1=O] |
| | | | 1-(4-chlorobenzyl)-3-(4-(((1-methyl-2-oxopyrrolidin-3- |
| | | | yl)amino)methyl)phenyl)urea. LCMS-APCI (POS.) |
| | | | m/z: 387 (M + H)⁺. |
| 17.4 | Intermediate 3.2 | 3- amino- 1- phenyl pyrrolidin- 2-one | 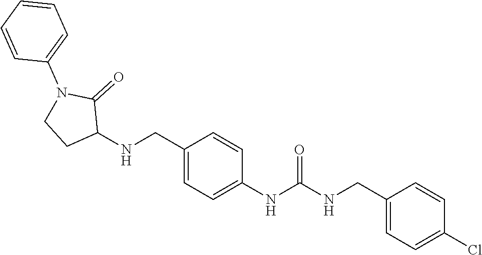 [SMILES: O=C(NCc1ccc(Cl)cc1)Nc1ccc(CNC2CCN(c3ccccc3)C2=O)cc1] |
| | | | 1-(4-chlorobenzyl)-3-(4-(((2-oxo-1-phenylpyrrolidin-3- |
| | | | yl)amino)methyl)phenyl)urea. LCMS-APCI (POS.) m/z: |
| | | | 449 (M + H)⁺. |
| 17.5 | Intermediate 3.1 | 3- amino tetrahydro thiophene 1,1- dioxide | 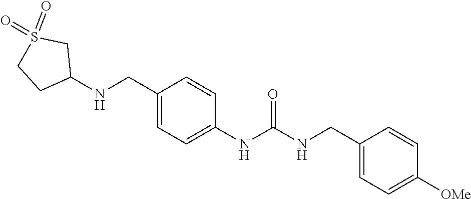 [SMILES: COc1ccc(CNC(=O)Nc2ccc(CNC3CCS(=O)(=O)C3)cc2)cc1] |
| | | | 1-(4-(((1,1-dioxidotetrahydrothiophen-3- |
| | | | yl)amino)methyl)phenyl)-3-(4-methoxybenzyl)urea. |
| | | | LCMS-APCI (POS.) m/z: 404 (M + H)⁺. |
| 17.6 | Intermediate 3.1 | 3- amino pyrrolidin- 2-one | 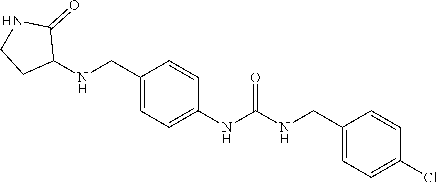 [SMILES: O=C(NCc1ccc(Cl)cc1)Nc1ccc(CNC2CCNC2=O)cc1] |
| | | | 1-(4-methoxybenzyl)-3-(4-(((2-oxopyrrolidin-3- |
| | | | yl)amino)methyl)phenyl)urea. LCMS-APCI (POS.) m/z: |
| | | | 369 (M + H)⁺. |

### Example R

### Synthesis of 1-(4-chlorobenzyl)-3-(4-(((1,1-dioxidotetrahydrothiophen-3-yl)(methyl)amino)methyl)phenyl)urea (Intermediate 18.1-18.2)

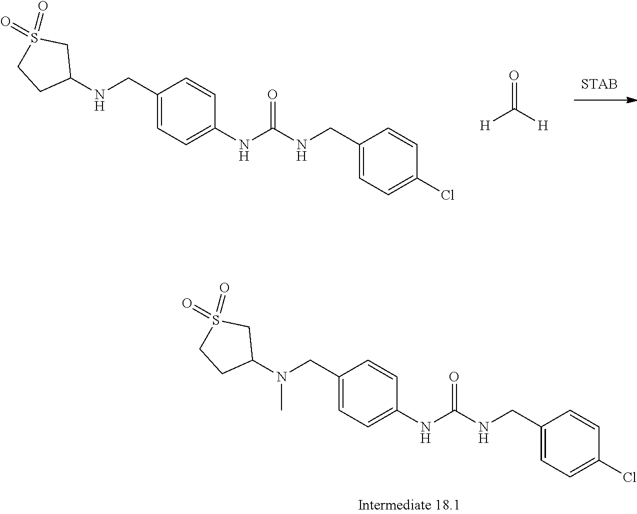

To a stirred mixture of 1-(4-chlorobenzyl)-3-(4-(((1,1-dioxidotetrahydrothiophen-3-yl)(methyl)amino)methyl)phenyl)urea (120.00 mg, 0.294 mmol, 1.00 equiv) and formaldehyde (53.00 mg, 1.765 mmol, 6 equiv) in DCE (4.00 mL) at 0° C. was added STAB (124.70 mg, 0.588 mmol, 2 equiv) and AcOH (35.33 mg, 0.588 mmol, 2 equiv). After stirred at r.t. for 2 h, the above mixture was added additional formaldehyde (53.00 mg, 1.765 mmol, 6 equiv), and STAB (124.70 mg, 0.588 mmol, 2 equiv). The resulting mixture was stirred at r.t. for overnight, adjusted pH to 10 with NH₃H₂O(2 mL), and extracted with DCM (10 mL) twice. The combined organic layers were washed twice with water (10 mL), dried over anhydrous Na₂SO₄, concentrated under reduced pressure, and purified by Prep-HPLC with the following conditions (Column: XBridge Prep OBD C18 Column, 30*150 mm 5 um; Mobile Phase A: Water(10 MMOL/L NH4HCO3), Mobile Phase B: ACN; Flow rate:60 mL/min; Gradient:26 B to 56 B in 9 min; 254 nm;) to afford 50 mg of 1-(4-chlorobenzyl)-3-(4-(((1,1-dioxidotetrahydrothiophen-3-yl)(methyl)amino)methyl)phenyl)urea (40.28%) as a white solid. LCMS-APCI (POS.) m/z: 422 (M+H)⁺.

Intermediate 18.2 was prepared in a similar manner as Intermediate 18.1

| Intermediate | Amine | Structure, Name and Data |
|---|---|---|
| 18.2 | Intermediate 17.5 | 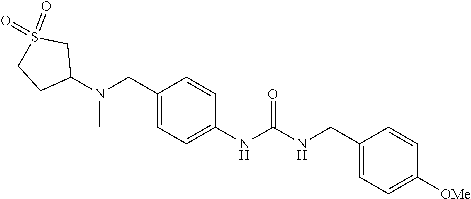 [SMILES: COc1ccc(CNC(=O)Nc2ccc(CN(C)C3CCS(=O)(=O)C3)cc2)cc1] |
| | | 1-(4-methoxybenzyl)-3-(4-(((1,1- |
| | | dioxidotetrahydrothiophen-3- |
| | | yl)(methyl)amino)methyl)phenyl)urea. LCMS-APCI |
| | | (POS.) m/z: 418 (M + H)⁺. |

### Example S

### Synthesis of tert-butyl (2-(4-nitrophenyl)-2-oxoethyl)carbamate (Intermediate 19)

### Step 1: Preparation of 2-amino-1-(4-nitrophenyl)ethan-1-one hydrochloride (Intermediate 19-a)

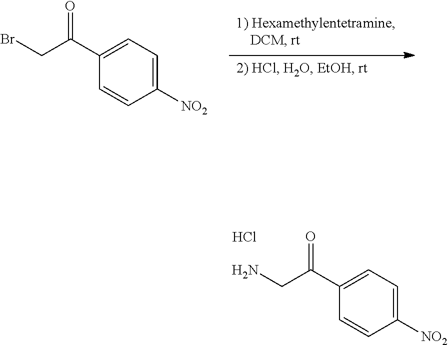

To a solution of 2-amino-1-(4-bromophenyl)ethanone (100.00 g, 467.154 mmol, 1.00 equiv) in DCM (1.20 L) was added Hexamethylentetramine (85.00 g, 607.143 mmol, 1.30 equiv). The resulting mixture was stirred at r.t. for 2 h. The precipitated solids were collected by filtration and washed with CH₂Cl₂ (500 mL). The residue was added HCl (200.00 mL, 6 mol/L) and EtOH (1.00 L). The resulting mixture was stirred at r.t. for 3 h, leaved overnight. The precipitated solids were collected by filtration and washed with hexane (500 mL), concentrated under vacuum to afford 140 g of 2-amino-1-(4-nitrophenyl)ethanone hydrochloride (crude) as a light yellow solid. LCMS-APCI (POS.) m/z: 181 (M+H)⁺.

### Step 2: Preparation of tert-butyl (2-(4-nitrophenyl)-2-oxoethyl)carbamate (Intermediate 19)

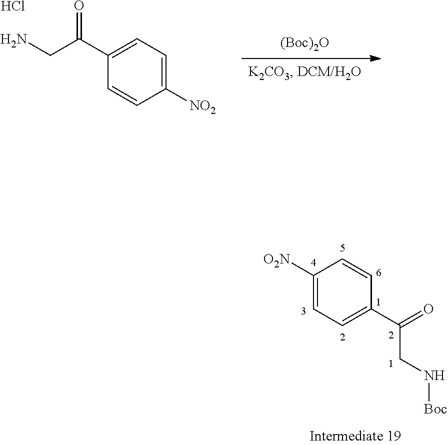 [SMILES: "(Boc)2O" ; Cl ; NCC(=O)c1ccc([N+](=O)[O-])cc1 ; "K2CO3, DCM/H2O" ; CC(C)(C)OC(=O)NCC(=O)c1ccc([N+](=O)[O-])cc1]

To a solution of 2-amino-1-(4-nitrophenyl)ethanone hydrochloride (140.00 g, 646.293 mmol, 1.00 equiv) in DCM (1.60 L) were added a solution of K₂CO₃ (179.00 g, 1295.173 mmol, 2.00 equiv) in H₂O (700.00 mL) and di-tert-butyl dicarbonate (169.00 g, 774.345 mmol, 1.20 equiv). The resulting mixture was stirred at r.t. for 3 h and extracted twice with CH₂Cl₂ (1 L). The combined organic layers were washed twice with brine (1 L), dried over anhydrous Na₂SO₄, concentrated under reduced pressure to afford 176 g of tert-butyl N-[2-(4-nitrophenyl)-2-oxoethyl]carbamate (crude) as a brown oil. LCMS-APCI (POS.) m/z: 225 (M+H−56)⁺.

### Example T

### Synthesis of 5-(4-nitrophenyl)piperazin-2-one (Intermediate 20)

### Step 1: Preparation of methyl (2-((tert-butoxycarbonyl)amino)-1-(4-nitrophenyl)ethyl)glycinate (Intermediate 20-a)

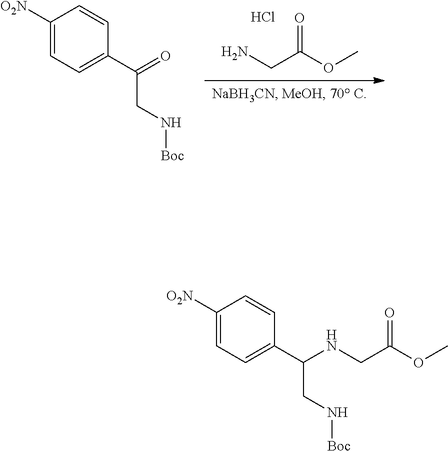

A solution of tert-butyl N-[2-(4-nitrophenyl)-2-oxoethyl] carbamate (14.00 g, 49.950 mmol, 1.00 equiv) and methyl 2-aminoacetate hydrochloride (12.61 g, 100.400 mmol, 2.01 equiv) in MeOH (200.00 mL) was stirred at r.t. for 30 min. Then the above resulting mixture at 0° C. was added NaBH₃CN (6.22 g, 98.901 mmol, 1.98 equiv). The resulting mixture was stirred at 70° C. for overnight, cooled to r.t., adjusted to pH 8 with saturated NH4. H2O (aq.) and extracted twice with EtOAc (200 mL). The combined organic layers were washed twice with water (200 mL), dried over anhydrous Na₂SO₄, concentrated under reduced pressure to afford 17 g (crude) of methyl 2-([2-[(tert-butoxycarbonyl)amino]-1-(4-nitrophenyl)ethyl]amino)acetate as a brown oil. LCMS-APCI (POS.) m/z: 354 (M+H)⁺.

### Step 2: Preparation of 2-((2-methoxy-2-oxoethyl)amino)-2-(4-nitrophenyl)ethan-1-aminium 2,2,2-trifluoroacetate (Intermediate 20-b)

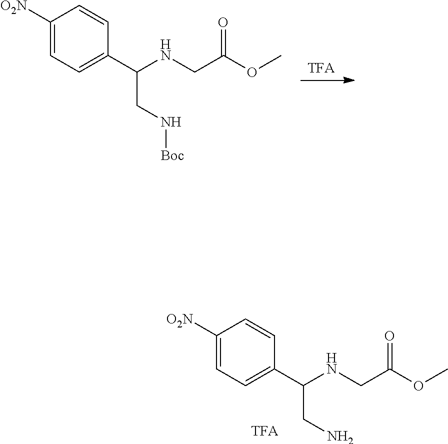 [SMILES: COC(=O)CNC(CNC(=O)OC(C)(C)C)c1ccc([N+](=O)[O-])cc1 ; "TFA" ; COC(=O)CNC(CN)c1ccc([N+](=O)[O-])cc1 ; "TFA"]

To a stirred solution of methyl 2-([2-[(tert-butoxycarbonyl) amino]-1-(4-nitrophenyl)ethyl]amino)acetate (17.00 g, 48.108 mmol, 1.00 equiv) in DCM (200.00 mL) at r.t. was added TFA (40.00 mL, 188.483 mmol, 20.18 equiv). The resulting mixture was stirred at r.t. for 1 h, concentrated under reduced pressure to afford 7 g (crude) of methyl 2-[[2-amino-1-(4-nitrophenyl)ethyl]amino]acetate TFA salt as a brown oil. LCMS-APCI (POS.) m/z: 254 (M+H)⁺.

### Step 3: Preparation of 5-(4-nitrophenyl)piperazin-2-one (Intermediate 20)

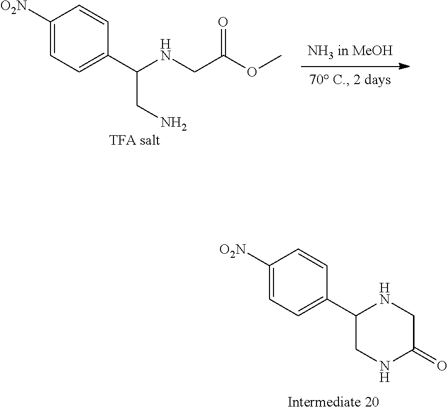

A solution of methyl 2-[[2-amino-1-(4-nitrophenyl) ethyl]amino]acetate TFA salt (7.00 g, 27.640 mmol, 1.00 equiv) in NH₃(g) in MeOH (70.00 mL) was stirred at 70° C. for 1 h. The mixture was cooled to r.t., concentrated under reduced pressure, purified by trituration with EtOAc (100 mL). The precipitated solids were collected by filtration and washed twice with EtOAc (100 mL), concentrated under reduced pressure to afford 2 g (32.71%) of 5-(4-nitrophenyl) piperazin-2-one as a brown solid. LCMS-APCI (POS.) m/z: 222 (M+H)⁺.

### Example U

### Synthesis of tert-butyl 4-methyl-2-(4-nitrophenyl)-5-oxopiperazine-1-carboxylate (Intermediate 21)

### Step 1: Preparation of tert-butyl 2-(4-nitrophenyl)-5-oxopiperazine-1-carboxylate (Intermediate 21-a)

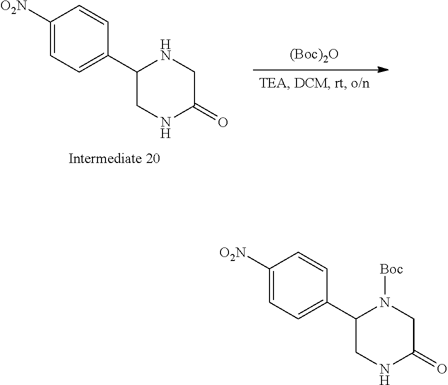 [SMILES: "(Boc)2O" ; O=C1CNC(c2ccc([N+](=O)[O-])cc2)CN1 ; "TEA, DCM, rt, o/n" ; "Intermediate 20" ; CC(C)(C)OC(=O)N1CC(=O)NCC1c1ccc([N+](=O)[O-])cc1]

To a stirred solution of 5-(4-nitrophenyl) piperazin-2-one (500.00 mg, 2.260 mmol, 1.00 equiv) in DCM (10.00 mL) were added (Boc)₂O (1479.87 mg, 6.781 mmol, 3 equiv) and TEA (914.85 mg, 9.041 mmol, 4 equiv). The resulting mixture was stirred at r.t. for overnight, added water (10 mL) and extracted twice with DCM (10 mL). The combined organic layers were washed twice with brine (10 mL), dried over anhydrous Na₂SO₄, concentrated under reduced pressure, purified by silica gel column chromatography, eluted with PE/EtOAc (3:2) to afford 380 mg (52.32%) of tert-butyl 2-(4-nitrophenyl)-5-oxopiperazine-1-carboxylate as a yellow oil. LCMS-APCI (POS.) m/z: 322 (M+H)⁺.

### Step 2: Preparation of tert-butyl 4-methyl-2-(4-nitrophenyl)-5-oxopiperazine-1-carboxylate (Intermediate 20)

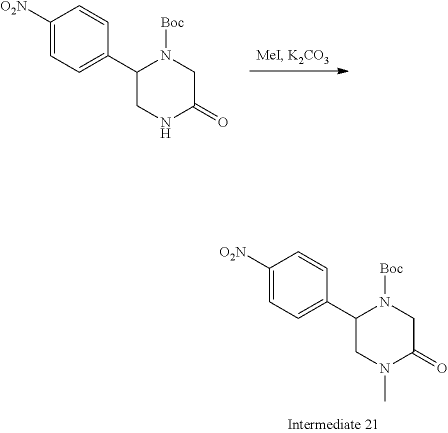

To a stirred solution of tert-butyl 2-(4-nitrophenyl)-5-oxopiperazine-1-carboxylate (350.00 mg, 1.089 mmol, 1.00 equiv) in DMF (8.00 mL) were added CH₃I (463.81 mg, 3.268 mmol, 3 equiv) and Cs₂CO₃ (1419.55 mg, 4.357 mmol, 4 equiv). The resulting mixture was stirred at r.t for 2 h, filtered to remove solids, the filtration was concentrated under reduced pressure, and purified by silica gel column chromatography, eluted with PE/EtOAc (5:2) to afford 230 mg (62.97%) of tert-butyl 4-methyl-2-(4-nitrophenyl)-5-oxopiperazine-1-carboxylate as a yellow oil. LCMS-APCI (POS.) m/z: 336 (M+H)⁺.

### Example V

### Synthesis of 4-methyl-5-(4-nitrophenyl)piperazin-2-one (Intermediate 22)

### Preparation of 4-methyl-5-(4-nitrophenyl)piperazin-2-one (Intermediate 22)

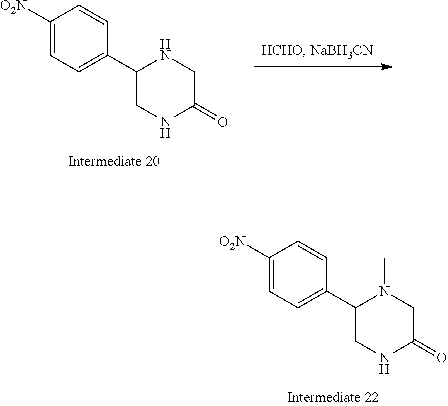 [SMILES: O=C1CNC(c2ccc([N+](=O)[O-])cc2)CN1 ; CN1CC(=O)NCC1c1ccc([N+](=O)[O-])cc1]

To a stirred solution of 5-(4-nitrophenyl) piperazin-2-one (600.00 mg, 2.712 mmol, 1.00 equiv) in MeOH (10.00 mL) were added HCHO (813.68 mg, 27.120 mmol, 10.00 equiv), NaBH₃CN (340.89 mg, 5.425 mmol, 2 equiv) and AcOH (530.00 mg, 8.826 mmol, 3.25 equiv). The resulting mixture was stirred at r.t. for 5 h, concentrated under reduced pressure, and purified by silica gel column chromatography, eluted with DCM/MeOH (20:1) to afford 800 mg of 4-methyl-5-(4-nitrophenyl)piperazin-2-one as a yellow solid LCMS-APCI (POS.) m/z: 236 (M+H)⁺.

### Example W

### Synthesis of 1,4-dimethyl-5-(4-nitrophenyl)piperazin-2-one (Intermediate 23)

### Preparation of 1,4-dimethyl-5-(4-nitrophenyl)piperazin-2-one (Intermediate 23)

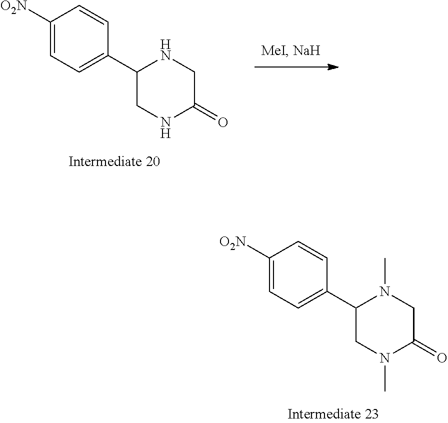 [SMILES: O=C1CNC(c2ccc([N+](=O)[O-])cc2)CN1 ; CN1CC(c2ccc([N+](=O)[O-])cc2)N(C)CC1=O]

To a stirred solution of 5-(4-nitrophenyl)piperazin-2-one (500.00 mg, 2.260 mmol, 1.00 equiv) in DMF (10.00 mL) at 0° C. was added NaH (361.60 mg, 9.041 mmol, 4.00 equiv, 60%). After stirred at 0° C. for 30 min, the resulting mixture at 0° C. was added CH₃I (962.45 mg, 6.781 mmol, 3.00 equiv). The resulting mixture was stirred at r.t. for overnight, and purified by C18 column chromatography, eluted with water (0.05% NH4HCO3)/ACN=(4:1) to afford 390 mg (69.22%) of 1,4-dimethyl-5-(4-nitrophenyl)piperazin-2-one as a brown solid. LCMS-APCI (POS.) m/z: 250 (M+H)⁺.

### Example X

### Synthesis of 1,4-dimethyl-6-(4-nitrophenyl)piperazin-2-one (Intermediate 24)

### Step 1: Preparation of tert-butyl (2-amino-2-(4-nitrophenyl)ethyl)carbamate (Intermediate 24-a)

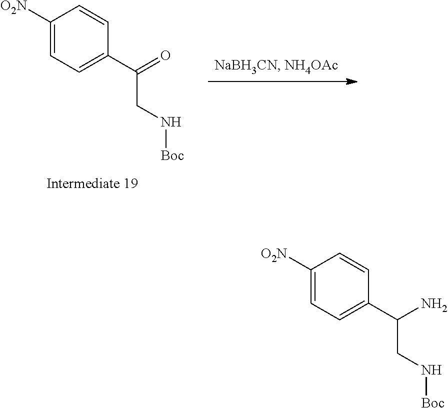 [SMILES: CC(C)(C)OC(=O)NCC(=O)c1ccc([N+](=O)[O-])cc1 ; CC(C)(C)OC(=O)NCC(N)c1ccc([N+](=O)[O-])cc1]

To a solution of tert-butyl N-[2-(4-nitrophenyl)-2-oxoethyl]carbamate (20.00 g, 71.357 mmol, 1.00 equiv) in MeOH (400.00 mL) at 0° C. were added NH₄OAc (14.00 g, 181.624 mmol, 2.55 equiv) and NaBH₃CN (110.00 g, 1750.422 mmol, 24.53 equiv). The resulting mixture was stirred at 70° C. for overnight, cooled to r.t., adjusted to pH 8 with saturated NH₃H₂O, extracted twice with CH₂Cl₂ (1 L). The combined organic layers were washed twice with brine (1 L), dried over anhydrous Na₂SO₄, concentrated under reduced pressure, purified by silica gel column chromatography, eluted with MeOH/EtOAc (1:20) to afford 6.7 g of tert-butyl N-[2-amino-2-(4-nitrophenyl)ethyl]carbamate (33.38%) as a brown oil and 2.8 g of tert-butyl (2-hydroxy-2-(4-nitrophenyl)ethyl)carbamate as a brown solid. LCMS-APCI (POS.) m/z: 226 (M+H−56)⁺.

### Step 2: Preparation of 1-(4-nitrophenyl)ethane-1,2-diamine (Intermediate 24-b)

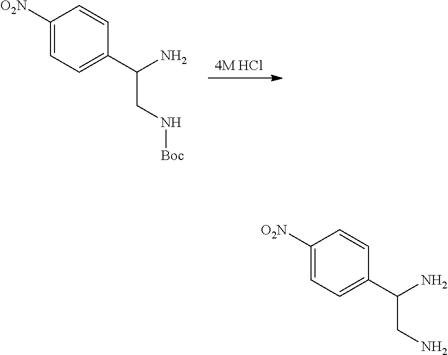 [SMILES: CC(C)(C)OC(=O)NCC(N)c1ccc([N+](=O)[O-])cc1 ; "4M HCl" ; NCC(N)c1ccc([N+](=O)[O-])cc1]

To a solution of tert-butyl N-[2-amino-2-(4-nitrophenyl)ethyl]carbamate (4.60 g, 16.371 mmol, 1.00 equiv) in DCM (40 mL) was added HCl(gas)in 1,4-dioxane (30.00 mL). The resulting mixture was stirred at r.t. for 3 h, adjusted PH to 13-14 with NaOH(aq), and extracted twice with DCM:MeOH (10:1, 50 mL). The combined organic layers were washed with brine (50 mL), dried over anhydrous Na₂SO₄, concentrated under reduced pressure to afford 2.3 g of 1-(4-nitrophenyl)ethane-1,2-diamine as a light brown oil. LCMS-APCI (POS.) m/z: 182 (M+H)⁺.

### Step 3: Preparation of 1-(4-nitrophenyl)ethane-1,2-diamine (Intermediate 24-c)

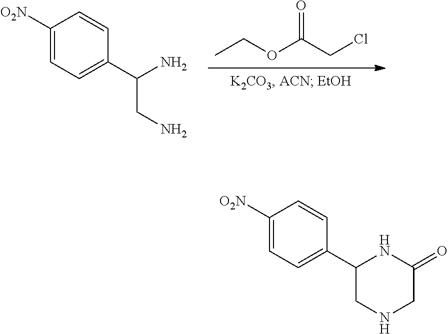

To a solution of 1-(4-nitrophenyl)ethane-1,2-diamine (2.60 g, 14.349 mmol, 1.00 equiv) in ACN (26.00 mL) were added K₂CO₃ (5.95 g, 43.052 mmol, 3.00 equiv) and ethyl chloroacetate (1.76 g, 14.349 mmol, 1.00 equiv). After stirred at r.t. for overnight, the resulting mixture was added EtOH (4.00 mL). The resulting mixture was stirred at 80° C. for 3 h, cooled to r.t., and filtered to remove solids. The filtration was concentrated under reduced pressure, and purified by silica gel column chromatography, eluted with DCM/MeOH (10:1) to afford 2.2 g of 6-(4-nitrophenyl)piperazin-2-one (69.31%) as a brown solid. LCMS-APCI (POS.) m/z: 222 (M+H)⁺.

### Step 4: Preparation of tert-butyl 3-(4-nitrophenyl)-5-oxopiperazine-1-carboxylate (Intermediate 24-d)

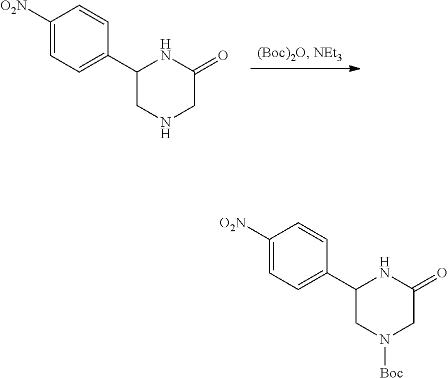

To a solution of 6-(4-nitrophenyl)piperazin-2-one (1.00 g, 4.520 mmol, 1.00 equiv) and TEA (914.00 mg, 9.033 mmol, 2.00 equiv) in DCM (10.00 mL) was added di-tert-butyl dicarbonate (1.18 g, 5.407 mmol, 1.20 equiv). The resulting mixture was stirred at r.t. for 2 h, and extracted twice with DCM (20 mL). The combined organic layers were washed twice with brine (20 mL), dried over anhydrous Na₂SO₄, concentrated under reduced pressure to afford 1.1 g of tert-butyl 3-(4-nitrophenyl)-5-oxopiperazine-1-carboxylate (crude) as a yellow semi-solid. LCMS-APCI (POS.) m/z: 266 (M+H−56)⁺.

### Step 5: Preparation of tert-butyl 4-methyl-3-(4-nitrophenyl)-5-oxopiperazine-1-carboxylate (Intermediate 24-e)

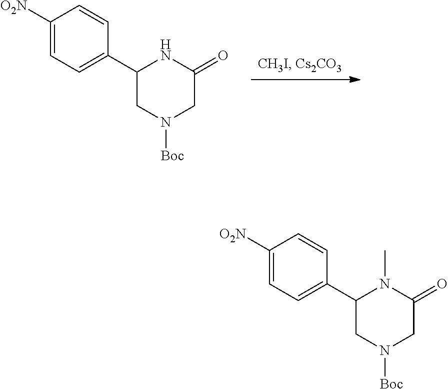

To a solution of tert-butyl 3-(4-nitrophenyl)-5-oxopiperazine-1-carboxylate (1.10 g, 3.423 mmol, 1.00 equiv) in DMF (25.00 mL) were added Cs₂CO₃ (2.20 g, 6.752 mmol, 1.97 equiv) and methyl iodide (534.48 mg, 3.766 mmol, 1.10 equiv). The resulting mixture was stirred at r.t. for 2 h, extracted twice with EtOAc (30 mL). The combined organic layers were washed twice with brine (30 mL), dried over anhydrous Na₂SO₄, concentrated under reduced pressure, purified by silica gel column chromatography, eluted with PE/EtOAc (1:4) to afford 530 mg of tert-butyl 4-methyl-3-(4-nitrophenyl)-5-oxopiperazine-1-carboxylate (46.17%) as a yellow semi-solid. LCMS-APCI (POS.) m/z: 300 (M+H−56)⁺.

### Step 6: Preparation of 1-methyl-6-(4-nitrophenyl)piperazin-2-one hydrochloride (Intermediate 24-f)

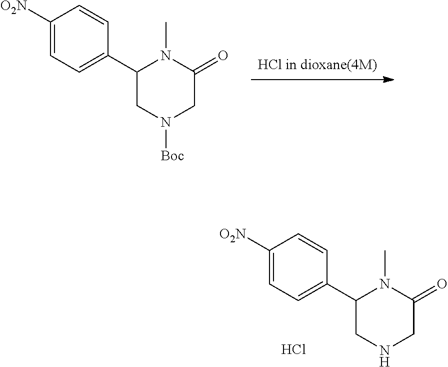 [SMILES: CN1C(=O)CN(C(=O)OC(C)(C)C)CC1c1ccc([N+](=O)[O-])cc1 ; "HCl in dioxane(4M)" ; CN1C(=O)CNCC1c1ccc([N+](=O)[O-])cc1 ; Cl]

To a solution of tert-butyl 4-methyl-3-(4-nitrophenyl)-5-oxopiperazine-1-carboxylate (530.00 mg, 1.580 mmol, 1.00 equiv) in DCM (8.00 mL) was added HCl(gas)in 1,4-dioxane (2.00 mL, 4 mol/L). The resulting mixture was stirred at r.t. for 2 h, concentrated under reduced pressure to afford 550 mg(crude) of 1-methyl-6-(4-nitrophenyl)piperazin-2-one hydrochloride as an orange semi-solid. LCMS-APCI (POS.) m/z: 236 (M+H)⁺.

### Step 7: Preparation of 1,4-dimethyl-6-(4-nitrophenyl)piperazin-2-one (Intermediate 24)

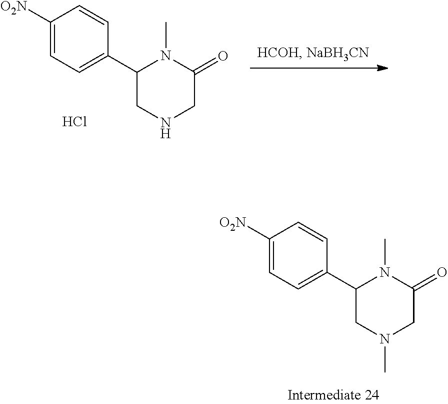

To a solution of tert-butyl 4-methyl-3-(4-nitrophenyl)-5-oxopiperazine-1-carboxylate (530.00 mg, 1.580 mmol, 1.00 equiv) in DCM (8.00 mL) was added HCl(gas)in 1,4-dioxane (2.00 mL, 4 mol/L). The resulting mixture was stirred at r.t. for 2 h, concentrated under reduced pressure to afford 550 mg(crude) of 1-methyl-6-(4-nitrophenyl)piperazin-2-one hydrochloride as an orange semi-solid. LCMS-APCI (POS.) m/z: 250 (M+H)⁺.

### Example Y

### Synthesis of 4-(2-fluoro-4-nitrobenzyl)-1-methylpiperazin-2-one (Intermediate 25.1-25.2)

### Preparation of 4-(2-fluoro-4-nitrobenzyl)-1-methylpiperazin-2-one (Intermediate 25.1)

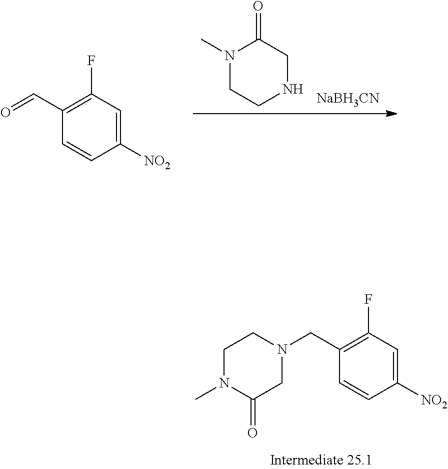

To a solution of 2-fluoro-4-nitrobenzaldehyde (200.00 mg, 1.183 mmol, 1.00 equiv) in MeOH (5.00 mL) was added 1-methylpiperazin-2-one (202.00 mg, 1.770 mmol, 1.50 equiv). After stirring at r.t. for 30 min, the mixture was added AcOH (142.00 mg, 2.365 mmol, 2.00 equiv) and NaBH₃CN (151.00 mg, 2.403 mmol, 2.03 equiv). The resulting mixture was stirred at r.t. for overnight, adjusted to pH 8 with NH₃. H₂O, concentrated under vacuum, and purified by C18 column chromatography, eluted with water(0.05% NH₄HCO₃)/ACN (2:1) to afford 80 mg of 4-[(2-fluoro-4-nitrophenyl)methyl]-1-methylpiperazin-2-one (25.31%) as a yellow oil. LCMS-APCI (POS.) m/z: 268 (M+H)⁺.

Intermediate 25.2 was prepared in a similar manner as Intermediate 25.1

| Inter- | | |
|---|---|---|
| mediate | Amine | Structure, Name and Data |
| 25.2 | 4- methyl piperazin- 2-one | 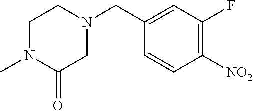 [SMILES: CN1CCN(Cc2ccc([N+](=O)[O-])c(F)c2)CC1=O] |
| | | 4-(3-fluoro-4-nitrobenzyl)-1-methylpiperazin-2- |
| | | one. LCMS-APCI (POS.) m/z: 268 (M + H)⁺. |

### Example Z

### Synthesis of 4-methyl-1-(1-(4-nitrophenyl)ethyl)piperazin-2-one (Intermediate 26)

### Step 1: Preparation of tert-butyl methyl(2-((1-(4-nitrophenyl)ethyl)amino)ethyl)carbamate (Intermediate 26-a)

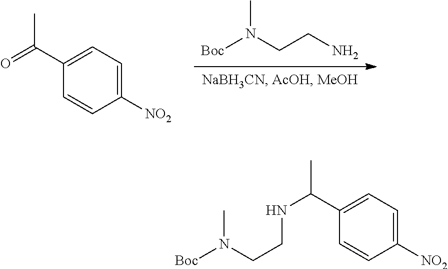

To a stirred solution of PNAP (2.27 g, 13.774 mmol, 1.2 equiv) and tert-butyl N-(2-aminoethyl)-N-methylcarbamate (2.00 g, 11.478 mmol, 1.00 equiv) in MeOH(30 mL) at 0° C. were added NaBH₃CN (1.44 g, 22.956 mmol, 2 equiv) and AcOH (1.38 g, 22.956 mmol, 2 equiv). The resulting mixture was stirred at r.t. for overnight, adjusted to pH 8 with saturated NH₄. H₂O (aq.), and extracted twice with EtOAc (50 mL). The combined organic layers were washed twice with water (50 mL), dried over anhydrous Na₂SO₄, concentrated under reduced pressure, and purified by silica gel column chromatography, eluted with PE/EtOAc (2:3) to afford 2.8 g (75.43%) of tert-butyl N-methyl-N-(2-[[1-(4-nitrophenyl)ethyl]amino]ethyl)carbamate as a light yellow oil. LCMS-APCI (POS.) m/z: 268 (M+H−56)⁺.

### Step 2: Preparation of tert-butyl (2-(2-chloro-N-(1-(4-nitrophenyl)ethyl)acetamido)ethyl)(methyl)carbamate (Intermediate 24-b)

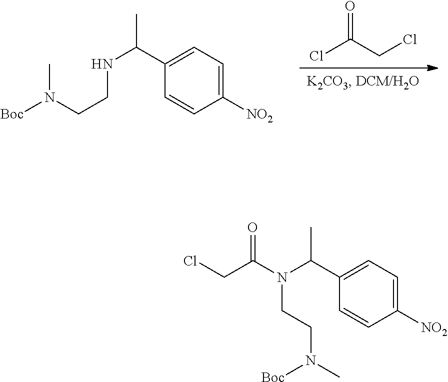

To a stirred solution of PNAP (2.27 g, 13.774 mmol, 1.2 equiv) and tert-butyl N-(2-aminoethyl)-N-methylcarbamate (2.00 g, 11.478 mmol, 1.00 equiv) in MeOH(30 mL) at 0° C. were added NaBH₃CN (1.44 g, 22.956 mmol, 2 equiv) and AcOH (1.38 g, 22.956 mmol, 2 equiv). The resulting mixture was stirred at r.t. for overnight, adjusted to pH 8 with saturated NH₄. H₂O (aq.), and extracted twice with EtOAc (50 mL). The combined organic layers were washed twice with water (50 mL), dried over anhydrous Na₂SO₄, concentrated under reduced pressure, and purified by silica gel column chromatography, eluted with PE/EtOAc (2:3) to afford 2.8 g (75.43%) of tert-butyl (2-(2-chloro-N-(1-(4-nitrophenyl)ethyl)acetamido)ethyl)(methyl)carbamate as a light yellow oil. LCMS-APCI (POS.) m/z: 400 (M+H)⁺.

### Step 3: Preparation of 2-chloro-N-(2-(methylamino)ethyl)-N-(1-(4-nitrophenyl)ethyl) acetamide hydrochloride (Intermediate 26-c)

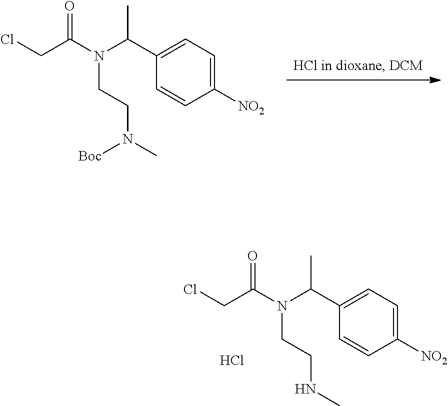 [SMILES: CC(c1ccc([N+](=O)[O-])cc1)N(CCN(C)C(=O)OC(C)(C)C)C(=O)CCl ; "HCl in dioxane, DCM" ; CNCCN(C(=O)CCl)C(C)c1ccc([N+](=O)[O-])cc1 ; Cl]

To a stirred solution of tert-butyl N-(2-[2-chloro-N-[1-(4-nitrophenyl)ethyl]acetamido]ethyl)-N-methylcarbamate (3.00 g, 7.502 mmol, 1.00 equiv) in DCM (30.00 mL) was added HCl(gas)in 1,4-dioxane (30.00 mL). The resulting mixture was stirred at r.t. for 1 h, concentrated under reduced pressure to afford 3.1 g of 2-chloro-N-[2-(methylamino)ethyl]-N-[1-(4-nitrophenyl)ethyl]acetamide hydrogen chloride as a light yellow solid. LCMS-APCI (POS.) m/z: 300 (M+H)⁺.

### Step 4: Preparation of 4-methyl-1-(1-(4-nitrophenyl)ethyl)piperazin-2-one (Intermediate 26)

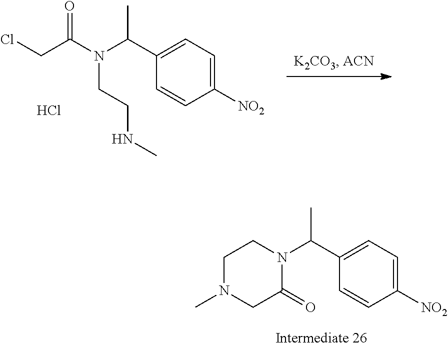

To a stirred solution of 2-chloro-N-[2-(methylamino) ethyl]-N-[1-(4-nitrophenyl)ethyl]acetamide hydrogen chloride (3.10 g, 10.342 mmol, 1.00 equiv) in ACN (50.00 mL) was added K₂CO₃ (7.15 g, 51.735 mmol, 5.00 equiv). The resulting mixture was stirred at 80° C. for 1 h, cooled to r.t., filtered to remove solids. The filtration was concentrated under reduced pressure to afford 1.69 g of 4-methyl-1-[1-(4-nitrophenyl) ethyl] piperazin-2-one as a yellow oil. LCMS-APCI (POS.) m/z: 264 (M+H)⁺.

### Example AA

### Synthesis of 1-(4-chlorobenzyl)-3-(4-((methyl(2-oxopyrrolidin-3-yl)amino)methyl)phenyl)urea (Intermediate 27.1-27.4)

### Step 1: Preparation of phenyl (4-formylphenyl)carbamate (Intermediate 27-a)

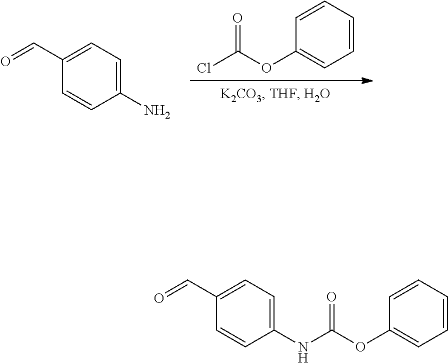

To a stirred solution of 4-aminobenzaldehyde (2.00 g, 16.510 mmol, 1.00 equiv) in THF (40.00 mL) at 0° C. were added a solution of K₂CO₃ (4.56 g, 32.994 mmol, 2.00 equiv) in H2O (10.00 mL) and phenyl chloroformate (3.87 g, 24.717 mmol, 1.50 equiv) dropwise over a period of 10 min. The resulting mixture was stirred at r.t. for 1 h, extracted with EtOAc (50 mL) twice. The combined organic layers were washed twice with brine(50 mL), dried over anhydrous MgSO4, concentrated under reduced pressure, and purified by silica gel column chromatography, eluted with PE/EtOAc (10:1) to afford 1.9 g of phenyl N-(4-formylphenyl)carbamate (84.41%) as a yellow LCMS-APCI (POS.) m/z: 242 (M+H)⁺.

### Step 2: Preparation of phenyl (4-(((2-oxopyrrolidin-3-yl)amino)methyl)phenyl)carbamate (Intermediate 27-b)

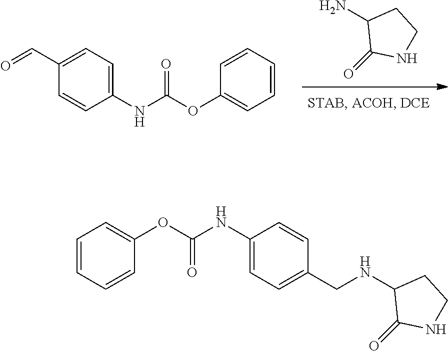 [SMILES: O=Cc1ccc(NC(=O)Oc2ccccc2)cc1 ; NC1CCNC1=O ; O=C(Nc1ccc(CNC2CCNC2=O)cc1)Oc1ccccc1]

To a stirred solution of phenyl N-(4-formylphenyl)carbamate (600.00 mg, 2.487 mmol, 1.00 equiv) in DCE (10.00 mL) were added 3-aminopyrrolidin-2-one (508.00 mg, 5.074 mmol, 2.04 equiv), STAB (1056.00 mg, 4.983 mmol, 2.00 equiv) and AcOH (299.00 mg, 4.979 mmol, 2.00 equiv). The resulting mixture was stirred at r.t. for overnight, and extracted with EtOAc(20 mL) twice. The combined organic layers were washed twice with brine(20 mL), dried over anhydrous MgSO4, concentrated under reduced pressure, purified by silica gel column chromatography, eluted with CH₂Cl₂/MeOH (12:1) to afford 415 mg of phenyl N-(4-[[(2-oxopyrrolidin-3-yl)amino]methyl]phenyl)carbamate (46.87%) as an off-white foam. LCMS-APCI (POS.) m/z: 326 (M+H)⁺.

### Step 3: Preparation of phenyl (4-((methyl(2-oxopyrrolidin-3-yl)amino)methyl)phenyl)carbamate (Intermediate 27-c)

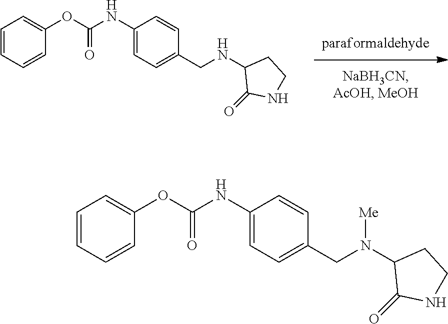 [SMILES: O=C(Nc1ccc(CNC2CCNC2=O)cc1)Oc1ccccc1 ; CN(Cc1ccc(NC(=O)Oc2ccccc2)cc1)C1CCNC1=O]

To a stirred solution of phenyl N-(4-[[(2-oxopyrrolidin-3-yl)amino]methyl]phenyl)carbamate (400.00 mg, 1.229 mmol, 1.00 equiv) in MeOH (8.00 mL, 197.591 mmol, 160.72 equiv) were added paraformaldehyde (369.00 mg, 4.096 mmol, 3.33 equiv) and NaBH₃CN (155.00 mg, 2.467 mmol, 2.01 equiv). The resulting mixture was stirred at r.t. for overnight, concentrated under reduced pressure, purified by silica gel column chromatography, eluted with PE/EtOAc(1:1) to afford 380 mg of phenyl (4-((methyl(2-oxopyrrolidin-3-yl)amino)methyl)phenyl)carbamate (79.65%) as an off-white solid. LCMS-APCI (POS.) m/z: 340 (M+H)⁺.

### Step 4: Preparation of 1-(4-chlorobenzyl)-3-(4-((methyl(2-oxopyrrolidin-3-yl)amino)methyl)phenyl)urea (Intermediate 27)

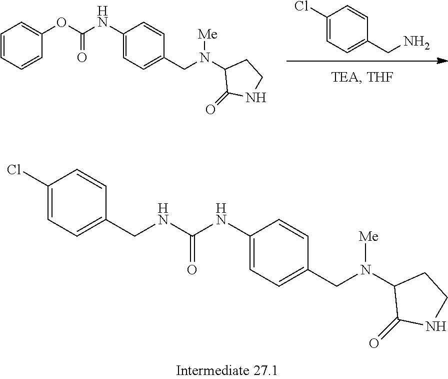 [SMILES: CN(Cc1ccc(NC(=O)Oc2ccccc2)cc1)C1CCNC1=O ; NCc1ccc(Cl)cc1 ; CN(Cc1ccc(NC(=O)NCc2ccc(Cl)cc2)cc1)C1CCNC1=O]

To a stirred solution of phenyl N-(4-[[methyl(2-oxopyrrolidin-3-yl)amino]methyl]phenyl)carbamate (100.00 mg, 0.295 mmol, 1.00 equiv) in THE (2.00 mL, 24.686 mmol, 83.78 equiv) were added TEA (149.00 mg, 1.472 mmol, 5.00 equiv) and 1-(4-chlorophenyl)methanamine (62.40 mg, 0.441 mmol, 1.50 equiv). The resulting mixture was stirred at 60° C. for overnight, concentrated under reduced pressure, purified by Prep-HPLC with the following conditions (2#SHIMADZU (HPLC-01)): Column, XBridge Prep OBD C₁₈ Column, 30*150 mm 5 um; mobile phase A: Water(10 MMOL/L NH4HCO3+0.1% NH3. H2O) and mobile phase B: ACN (25% Phase B up to 55% in 8 min); Detector, uv 254 nm. to afford 60 mg of 3-[(4-chlorophenyl)methyl]-1-(4-[[methyl(2-oxopyrrolidin-3-yl)amino]methyl]phenyl)urea (52.64%) as a white solid. LCMS-APCI (POS.) m/z: 387 (M+H)⁺.

Intermediates 27.2-27.4 were prepared in a similar manner as Intermediate 27.1

| | | Benzyl | |
|---|---|---|---|
| Intermediate | Amine | amine | Structure, Name and Data |
| 27.2 | 3-amino- 1- methyl pyrrolidin- 2-one | 4- Chloro benzyl amine | 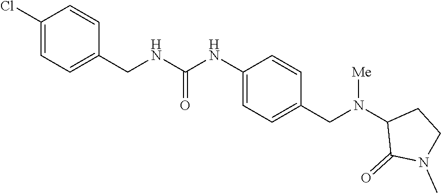 [SMILES: CN1CCC(N(C)Cc2ccc(NC(=O)NCc3ccc(Cl)cc3)cc2)C1=O] |
| | | | 1-(4-chlorobenzyl)-3-(4-((methyl(1-methyl-2- |
| | | | oxopyrrolidin-3-yl)amino)methyl)phenyl)urea. |
| | | | LCMS-APCI (POS.) m/z: 401 (M + H)⁺. |
| 27.3 | 3-amino- 1- methyl pyrrolidin- 2-one | 4- fluoro benzyl amine | 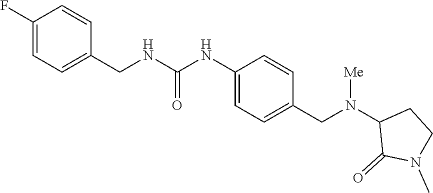 [SMILES: CN1CCC(N(C)Cc2ccc(NC(=O)NCc3ccc(F)cc3)cc2)C1=O] |
| | | | 1-(4-fluorobenzyl)-3-(4-((methyl(1-methyl-2- |
| | | | oxopyrrolidin-3-yl)amino)methyl)phenyl)urea. |
| | | | LCMS-APCI (POS.) m/z: 385 (M + H)⁺. |
| 27.4 | 3-amino- 1- methyl pyrrolidin- 2-one | 4- methoxy benzyl amine | 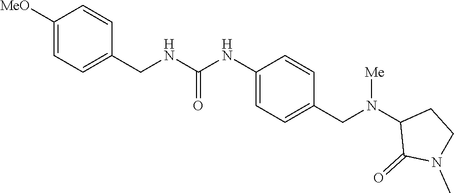 [SMILES: COc1ccc(CNC(=O)Nc2ccc(CN(C)C3CCN(C)C3=O)cc2)cc1] |
| | | | 1-(4-methoxybenzyl)-3-(4-((methyl(1-methyl-2- |
| | | | oxopyrrolidin-3-yl)amino)methyl)phenyl)urea. |
| | | | LCMS-APCI (POS.) m/z: 397 (M + H)⁺. |

### Example BB

### Synthesis of 5-(4-nitrophenyl)oxazolidin-2-one (Intermediate 28)

### Step 1: Preparation of tert-butyl (2-hydroxy-2-(4-nitrophenyl)ethyl)carbamate (Intermediate 28-a)

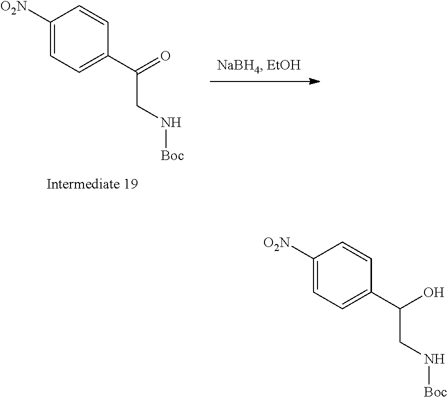 [SMILES: CC(C)(C)OC(=O)NCC(=O)c1ccc([N+](=O)[O-])cc1 ; CC(C)(C)OC(=O)NCC(O)c1ccc([N+](=O)[O-])cc1]

To a solution of tert-butyl N-[2-(4-nitrophenyl)-2-oxoethyl]carbamate(5.00 g, 17.839 mmol, 1.00 equiv) in EtOH(100.00 mL) at 0° C. was added NaBH₄(1.02 g, 26.961 mmol, 1.51 equiv). The resulting mixture was stirred at r.t. for 1 h under nitrogen atmosphere, concentrated under reduced pressure, and extracted third with EtOAc (50 mL). The combined organic layers were washed third with brine (50 mL), dried over anhydrous Na₂SO₄, concentrated under reduced pressure, and purified by silica gel column chromatography, eluted with PE/EtOAc (4:1) to afford 1.2 g of tert-butyl N-[2-hydroxy-2-(4-nitrophenyl)ethyl]carbamate as yellow solid. LCMS-APCI (POS.) m/z: 227 (M+H−56)⁺.

### Step 2: Preparation of 2-amino-1-(4-nitrophenyl)ethan-1-ol hydrochloride (Intermediate 28-b)

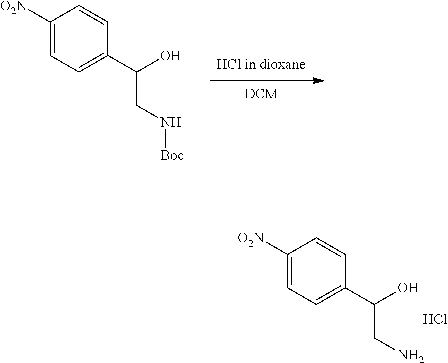

To a solution of tert-butyl N-[2-hydroxy-2-(4-nitrophenyl)ethyl]carbamate(2.10 g, 7.439 mmol, 1.00 equiv) in DCM(22.00 mL) was added HCl(gas)in 1,4-dioxane(5.50 mL, 96.346 mmol, 12.95 equiv). The resulting mixture was stirred at r.t. for overnight under nitrogen atmosphere, concentrated under reduced pressure to afford 1.9 g of 2-amino-1-(4-nitrophenyl)ethanol hydrochloride as an orange solid. LCMS-APCI (POS.) m/z: 183 (M+H)⁺.

### Step 3: Preparation of 5-(4-nitrophenyl)oxazolidin-2-one (Intermediate 28)

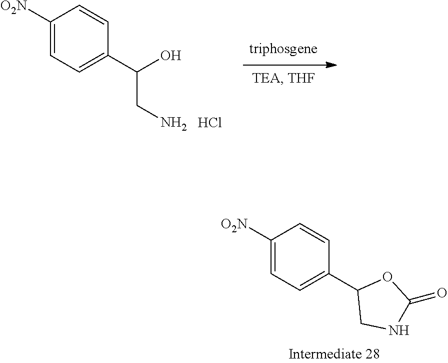 [SMILES: NCC(O)c1ccc([N+](=O)[O-])cc1 ; "TEA, THF" ; Cl ; O=C1NCC(c2ccc([N+](=O)[O-])cc2)O1]

To a solution of 2-amino-1-(4-nitrophenyl)ethanol hydrochloride(800.00 mg, 3.659 mmol, 1.00 equiv) and TEA(1.59 g, 15.713 mmol, 4.29 equiv) in THF (10.00 mL) at 0° C. was added triphosgene(309.00 mg, 1.041 mmol, 0.28 equiv). The resulting mixture was stirred at r.t. for 2 h under nitrogen atmosphere, quenched with MeOH (30 mL) at 0° C., concentrated under reduced pressure to afford 700 mg of 5-(4-nitrophenyl)-1,3-oxazolidin-2-one as a red solid. LCMS-APCI (POS.) m/z: 209 (M+H)⁺.

### Example CC

### Synthesis of 4-(2-fluoro-4-nitrobenzyl)-1-methylpiperazin-2-one (Intermediate 29)

### Preparation of 3-methyl-5-(4-nitrophenyl)oxazolidin-2-one (Intermediate 29)

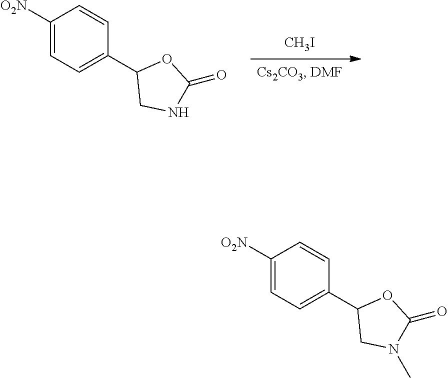

To a stirred solution of 5-(4-nitrophenyl)-1,3-oxazolidin-2-one(980.00 mg, 4.708 mmol, 1.00 equiv) in DMF(20.00 mL) were added Cs₂CO₃(6.13 g, 18.814 mmol, 4.00 equiv) and CH₃I(736.00 mg, 5.185 mmol, 1.10 equiv). The resulting mixture was stirred at r.t. for 4 h, and extracted with EtOAc (50 mL) third. The combined organic layers were washed third with brine (50 mL), dried over anhydrous Na₂SO₄, concentrated under reduced pressure, and purified by silica gel column chromatography, eluted with PE/EtOAc (3:2) to afford 330 mg of 3-methyl-5-(4-nitrophenyl)-1,3-oxazolidin-2-one as yellow solid. LCMS-APCI (POS.) m/z: 223 (M+H)⁺.

### Example DD

### Synthesis of 5-(4-nitrophenyl)oxazolidin-2-one (Intermediate 30.1-30.2)

### Step 1: Preparation of methyl 4-oxo-4-(pyridin-3-yl)butanoate (Intermediate 30-a)

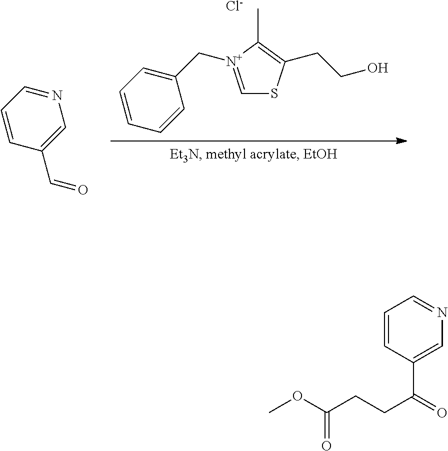 [SMILES: [Cl-] ; O=Cc1cccnc1 ; Cc1c(CCO)sc[n+]1Cc1ccccc1 ; "Et3N, methyl acrylate, EtOH" ; COC(=O)CCC(=O)c1cccnc1]

To a solution of 3-pyridinecarboxaldehyde(5.00 g, 46.7 mmol, 1.00 equiv) and methyl acrylate(4.80 g, 56.0 mmol, 1.20 equiv) in EtOH(50 mL) were added Et₃N(9.40 g, 93 mmol, 2.00 equiv) and 3-Benzyl-5-(hydroxyethyl)-4-methylthiazolium chloride(1.26 g, 4.67 mmol, 0.10 equiv) under nitrogen atmosphere. The resulting mixture was stirred at 50° C. for overnight under nitrogen atmosphere, cooled down to r.t., concentrated under reduced pressure, and extracted twice with EtOAc (100 mL). The combined organic layers were washed with brine (100 mL), dried over anhydrous Na₂SO₄, concentrated under reduced pressure, and purified by silica gel column chromatography, eluted with PE/EtOAc (4:1) to afford 2.19 g of methyl 4-oxo-4-(pyridin-3-yl)butanoate as a yellow solid. LCMS-APCI (POS.) m/z: 194 (M+H)⁺.

### Step 2: Preparation of 1-(4-nitrobenzyl)-5-(pyridin-3-yl)pyrrolidin-2-one (Intermediate 30.1)

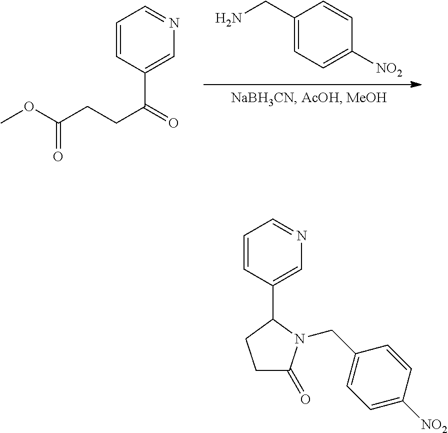

To a solution of methyl 4-oxo-4-(pyridin-3-yl)butanoate(1.72 g, 8.9 mmol, 1.00 equiv) and P-nitrobenzylamine(2.00 g, 10.688 mmol, 1.20 equiv) in MeOH(20.00 mL) at 0° C. were added NaBH₃CN(2.80 g, 17.8 mmol, 2.00 equiv) and AcOH(2.67 g, 17.8 mmol, 2.00 equiv). The resulting mixture was stirred at 70° C. for two days, cooled down to r.t., concentrated under reduced pressure, and extracted twice with EtOAc (50 mL). The combined organic layers were washed twice with brine (50 mL), dried over anhydrous Na₂SO₄, concentrated under reduced pressure, and purified by silica gel column chromatography, eluted with EtOAc to afford 1 g of 1-[(4-nitrophenyl)methyl]-5-(pyridin-3-yl)pyrrolidin-2-one as a light yellow oil. LCMS-APCI (POS.) m/z: 298 (M+H)⁺.

Intermediate 30.2 was prepared in a similar manner as Intermediate 30.1

| Inter- | | |
|---|---|---|
| mediate | Aldehye | Structure, Name and Data |
| 30.2 | 5- fluoro pyridine- 3- carbaldehyde | 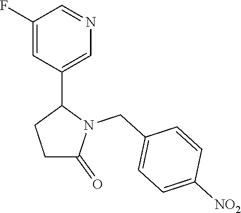 [SMILES: O=C1CCC(c2cncc(F)c2)N1Cc1ccc([N+](=O)[O-])cc1] |
| | | 5-(5-fluoropyridin-3-yl)-1-(4-nitrobenzyl) |
| | | pyrrolidin-2-one. |
| | | LCMS-APCI (POS.) m/z: 316 (M + H)⁺. |

### Example EE

### Synthesis of 1-(1-(4-nitrophenyl)ethyl)piperidin-2-one (Intermediate 31)

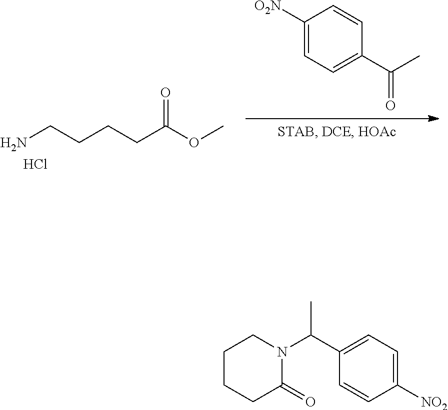

To a stirred mixture of methyl 5-aminopentanoate hydrochloride(1.00 g, 0.60 mmol, 1.00 equiv) and PNAP(1.300 g, 0.79 mmol, 1.32 equiv) in DCE(10.00 mL) were added STAB(2.500 g, 1.18 mmol, 1.98 equiv) and AcOH(700 mg, 1.17 mmol, 1.95 equiv). The resulting mixture was stirred at r.t. for 2 days, adjusted to pH 8 with saturated NaHCO₃ (aq.), and extracted twice with EtOAc (20 mL). The combined organic layers were washed twice with brine (20 mL), dried over anhydrous Na₂SO₄, concentrated under reduced pressure, and purified by silica gel column chromatography, eluted with PE/EtOAc (1:8) to afford 1 g of 1-[1-(4-nitrophenyl)ethyl]piperidin-2-one(67.52%) as a yellow solid. LCMS-APCI (POS.) m/z: 249 (M+H)⁺.

### Example FF

### Synthesis of 1-(4-(1,1-dioxidothiomorpholin-3-yl)phenyl)-3-(4-methoxybenzyl)urea (Intermediate 32)

### Step 1: Preparation of tert-butyl (2-((2-(4-nitrophenyl)-2-oxoethyl)thio)ethyl)carbamate (Intermediate 32-a)

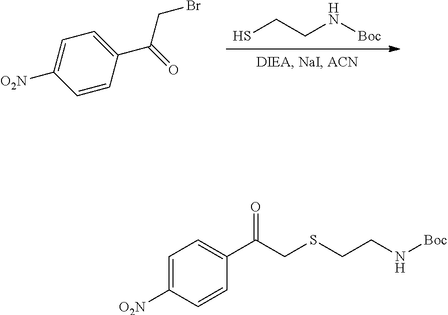

To a stirred solution of 2-bromo-1-(4-nitrophenyl)ethanone(8.00 g, 32.781 mmol, 1.00 equiv) and DIEA(8.47 g, 65.562 mmol, 2.00 equiv) in ACN(80.00 mL) at 0° C. were added NaI(1.47 g, 9.834 mmol, 0.30 equiv) and 2-bromo-1-(4-nitrophenyl)ethanone(8.00 g, 32.781 mmol, 1.00 equiv). The resulting mixture was stirred at r.t. for overnight under nitrogen atmosphere. The reaction was determined by LCMS. Water (200 mL) was added and the mixture was adjusted to pH 7 with HCl (aq.), and extracted three times with EtOAc (200 mL). The combined organic layers were washed with brine (200 mL), dried over anhydrous Na₂SO₄, concentrated under reduced pressure, and purified by silica gel column chromatography, eluted with PE/EtOAc (20:1) to afford 8.9 g of tert-butyl N-(2-[[2-(4-nitrophenyl)-2-oxoethyl]sulfanyl]ethyl)carbamate(79.76%) as a yellow solid. LCMS-APCI (POS.) m/z: 285 (M+H−56)⁺.

### Step 2: Preparation of tert-butyl (2-((2-(4-nitrophenyl)-2-oxoethyl)sulfonyl)ethyl)carbamate (Intermediate 32-b)

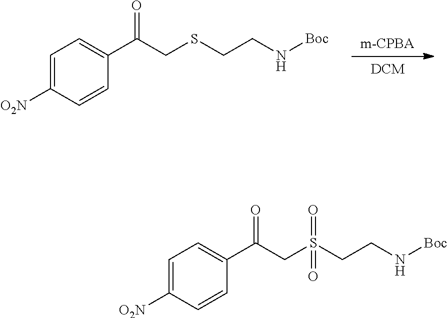

To a stirred mixture of tert-butyl N-(2-{[2-(4-nitrophenyl)-2-oxoethyl]sulfanyl}ethyl)carbamate (8.9 g, 26.092 mmol/L 1 equiv) in DCM (100 mL) was added m-CPBA (22.586 g, 130.419 mmol/L, 5 equiv). The resulting mixture was stirred at r.t. for overnight, added water(100 mL), and extracted three times with EtOAc (200 mL). The combined organic layers were washed with brine (100 mL), dried over anhydrous Na₂SO₄, concentrated under reduced pressure, and purified by silica gel column chromatography, eluted with PE/EA (4:1) to afford 7 g of tert-butyl N-{2-[2-(4-nitrophenyl)-2-oxoethanesulfonyl]ethyl}carbamate as a light yellow solid. LCMS-APCI (POS.) m/z: 317 (M+H−56)⁺.

### Step 3: Preparation of tert-butyl (2-((2-(4-aminophenyl)-2-oxoethyl)sulfonyl)ethyl)carbamate (Intermediate 32-c)

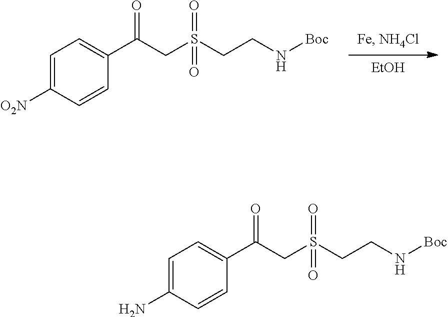

To a stirred mixture of tert-butyl N-{2-[2-(4-nitrophenyl)-2-oxoethanesulfonyl]ethyl}carbamate (7 g, 18.761 mmol/L, 1 equiv) in EtOH (80 mL) were added iron (4.2 g, 75.061 mmol/L, 4 equiv) and a solution of NH₄Cl (6.9 g, 131.327 mmol/L, 7 equiv) in H₂O (16 mL). The resulting mixture was stirred at r.t. for overnight under nitrogen atmosphere, concentrated under reduced pressure, purified by silica gel column chromatography, eluted with CH₂Cl₂/MeOH (50:1) to afford 5.3 g of tert-butyl N-{2-[2-(4-aminophenyl)-2-oxoethanesulfonyl]ethyl}carbamate (77.69%) as a light yellow solid. LCMS-APCI (POS.) m/z: 287 (M+H−56)⁺.

### Step 4: Preparation of tert-butyl (2-((2-oxo-2-(4-((phenoxycarbonyl)amino)phenyl)ethyl)sulfonyl)ethyl)carbamate (Intermediate 32-d)

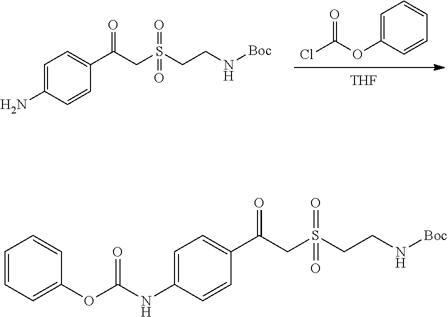 [SMILES: CC(C)(C)OC(=O)NCCS(=O)(=O)CC(=O)c1ccc(N)cc1 ; O=C(Cl)Oc1ccccc1 ; "THF" ; CC(C)(C)OC(=O)NCCS(=O)(=O)CC(=O)c1ccc(NC(=O)Oc2ccccc2)cc1]

To a solution of tert-butyl N-{2-[2-(4-aminophenyl)-2-oxoethanesulfonyl]ethyl}carbamate (3 g, 8.761 mmol, 1.00 equiv) in THE (30 mL) at 0° C. was added phenyl chloroformate (2.05 g, 13.093 mmol, 1.49 equiv) dropwise over a period of 10 min. The resulting mixture was stirred at r.t. for 2 h and then gradually warmed to 80° C. and stirred at 80° C. for overnight, cooled to r.t., concentrated under reduced pressure, purified by trituration with hexane:EA=10:1 (30 mL) and concentrated under reduced pressure to afford 3.7 g of phenyl N-[4-(2-{2-[(tert-butoxycarbonyl)amino]ethanesulfonyl}acetyl)phenyl]carbamate (91.31%) as a brown solid. LCMS-APCI (POS.) m/z: 407 (M+H−56)⁺.

### Step 5: Preparation of tert-butyl (2-((2-(4-(3-(4-methoxybenzyl)ureido)phenyl)-2-oxoethyl)sulfonyl)ethyl)carbamate (Intermediate 32-e)

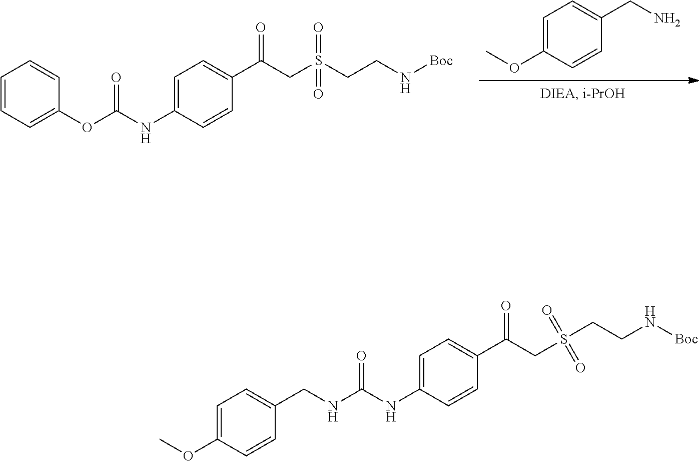

To a solution of phenyl N-[4-(2-{2-[(tert-butoxycarbonyl)amino]ethanesulfonyl}acetyl)phenyl]carbamate (1.1 g, 2.378 mmol, 1.00 equiv) in i-PrOH (11 mL) were added 4-methoxy-benzenemethanamine (0.4 g, 2.916 mmol, 1.23 equiv) and DIEA (0.9 g, 6.964 mmol, 2.93 equiv). The resulting mixture was stirred at 80° C. for 4 h, cooled to r.t., and purified by trituration with PE:EA=8:1 (15 mL) and concentrated under reduced pressure to afford 1.38 g of tert-butylN-(2-{2-[4-({[(4-methoxyphenyl)methyl]carbamoyl}amino)phenyl]-2-oxoethanesulfonyl}ethyl)carbamate(crude) as a brown solid. LCMS-APCI (POS.) m/z: 450 (M+H−56)⁺.

### Step 6: Preparation of 1-(4-(1,1-dioxidothiomorpholin-3-yl)phenyl)-3-(4-methoxybenzyl)urea (Intermediate 32)

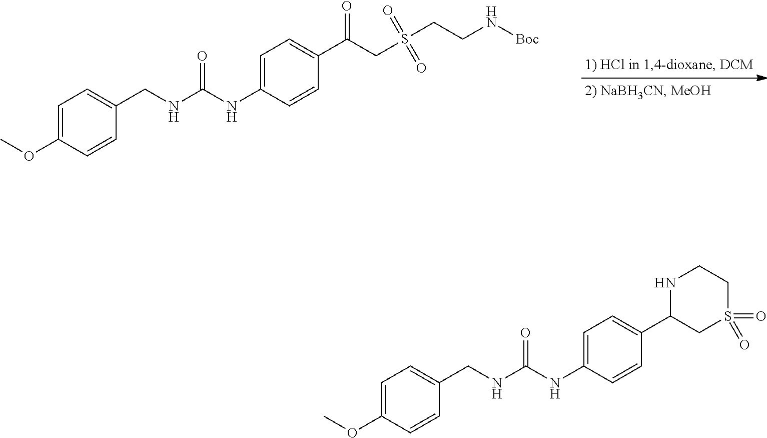 [SMILES: COc1ccc(CNC(=O)Nc2ccc(C(=O)CS(=O)(=O)CCNC(=O)OC(C)(C)C)cc2)cc1 ; COc1ccc(CNC(=O)Nc2ccc(C3CS(=O)(=O)CCN3)cc2)cc1]

To a solution of tert-butyl N-(2-{2-[4-({[(4-methoxyphenyl)methyl]carbamoyl}amino)phenyl]-2-oxoethanesulfonyl}ethyl)carbamate (1.28 g, 2.532 mmol, 1.00 equiv) in DCM (12 mL) was added HCl(gas)in 1,4-dioxane (3 mL, 4 mol/L). After stirred at r.t. for 2 h, the resulting mixture was concentrated under reduced pressure, added NaBH₃CN (0.32 g, 5.092 mmol, 2.01 equiv) and MeOH (12 mL). The above resulting mixture was stirred at r.t. for 2 h, added water (30 mL) and extracted three times with EtOAc (20 mL). The combined organic layers were washed twice with brine (20 ml), dried over anhydrous Na₂SO₄, concentrated under reduced pressure to afford 1.1 g(crude) of 1-[4-(1,1-dioxo-1lambda6-thiomorpholin-3-yl)phenyl]-3-[(4-methoxyphenyl)methyl]urea as a brown solid. The crude product (500 mg) was purified by Prep-HPLC with the following conditions (2#SHIMADZU (HPLC-01)): Column, YMC-Actus Triart C₁₈ ExRS, 30*150 mm, 5 μm; mobile phase, Water (10 mmol/L NH4HCO3+0.1% NH3. H2O) and ACN (15% ACN up to 45% in 10 min); Detector, UV254 nm, 210 nm to afford 230 mg of 1-[4-(1,1-dioxo-1lambda6-thiomorpholin-3-yl)phenyl]-3-[(4-methoxyphenyl)methyl]urea as a white solid. LCMS-APCI (POS.) m/z: 390 (M+H)⁺.

### Example GG

### Synthesis of 1-(4-(1,1-dioxidothiomorpholin-2-yl)phenyl)-3-(4-methoxybenzyl)urea (Intermediate 33)

### Step 1: Preparation of methyl 2-bromo-2-(4-nitrophenyl)acetate (Intermediate 33-a)

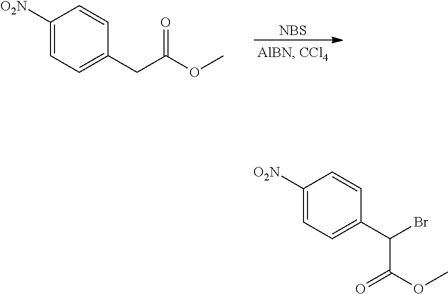

To a stirred solution of methyl 2-(4-nitrophenyl)acetate (5 g, 25.618 mmol, 1.00 equiv) and AIBN (0.21 g, 1.281 mmol, 0.05 equiv) in CCl₄ (50 mL) were added NBS (6.84 g, 38.427 mmol, 1.5 equiv). The resulting mixture was stirred at 80° C. for overnight, cooled to r.t., added water (100 mL) and extracted twice with CH2Cl2 (50 mL). The combined organic layers were washed twice with brine (100 mL), dried over anhydrous Na₂SO₄, concentrated under reduced pressure, and purified by C₁₈ column chromatography, eluted with water (0.05% NH4HCO3)/ACN (1:1) to afford 4.1 g (58.39%) of methyl 2-bromo-2-(4-nitrophenyl)acetate as a yellow oil. LCMS-APCI (POS.) m/z: 274 (M+H)⁺. ¹H NMR (300 MHz, DMSO-d₆) δ 8.34-8.19 (m, 2H), 7.89-7.78 (m, 2H), 6.17 (s, 1H), 3.76 (s, 3H).

### Step 2: Preparation of 2-(4-nitrophenyl)thiomorpholin-3-one (Intermediate 33-b)

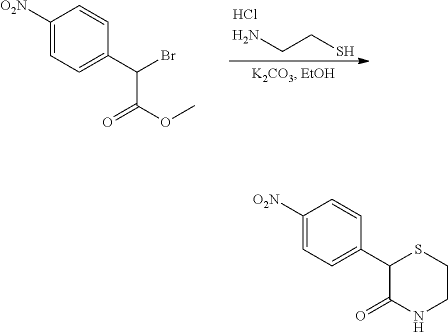

To a stirred solution of methyl 2-bromo-2-(4-nitrophenyl)acetate (3 g, 10.946 mmol, 1.00 equiv) in EtOH (30 mL) were added cysteamine hydrochloride (1.37 g, 12.041 mmol, 1.1 equiv) and K₂CO₃ (3.33 g, 24.081 mmol, 2.2 equiv). The resulting mixture was stirred at r.t. for overnight, added water (50 mL) and extracted twice with EA(100 mL). The combined organic layers were washed twice with brine (100 mL), dried over anhydrous Na₂SO₄, concentrated under reduced pressure and purified by silica gel column chromatography, eluted with PE/EA (1:9) to afford 1.4 g 53.68% of 2-(4-nitrophenyl)thiomorpholin-3-one as a yellow solid. LCMS-APCI (POS.) m/z: 239 (M+H)⁺.

### Step 3: Preparation of 2-(4-nitrophenyl)thiomorpholine (Intermediate 33-c)

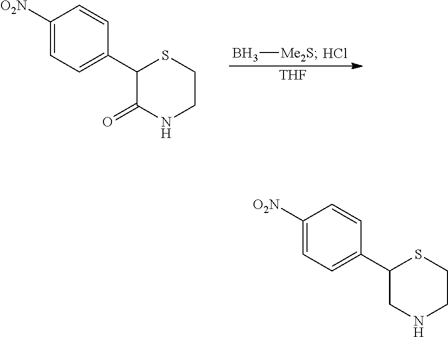

To a stirred solution of 2-(4-nitrophenyl)thiomorpholin-3-one (1.4 g, 5.876 mmol, 1.00 equiv) in THE (15 mL) was added BH₃-Me₂S (2.94 mL, 29.380 mmol, 5 equiv, 2 mol/L). The resulting mixture was stirred at 60° C. for 1 h, concentrated under reduced pressure, the residue was added HCl (15 mL, 4N) and stirred at 60° C. for additional 30 min. The mixture was adjusted to pH 8 with saturated NaHCO₃ (aq.), concentrated under reduced pressure, purified by C₁₈ column chromatography, eluted with water (0.05% NH₄HCO₃)/ACN (2:1) to afford 590 mg (44.77%) of 2-(4-nitrophenyl)thiomorpholine as a red oil. LCMS-APCI (POS.) m/z: 225 (M+H)⁺.

### Step 4: Preparation of tert-butyl 2-(4-nitrophenyl)thiomorpholine-4-carboxylate (Intermediate 33-d)

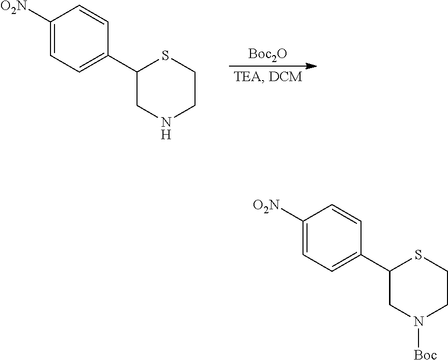

To a stirred solution of 2-(4-nitrophenyl)thiomorpholine (590 mg, 2.631 mmol, 1.00 equiv) and TEA (798.58 mg, 7.893 mmol, 3 equiv) in DCM(6 mL) were added (Boc)₂O (1148.26 mg, 5.262 mmol, 2 equiv). The resulting mixture was stirred at r.t. for overnight, concentrated under reduced pressure, purified by silica gel column chromatography, eluted with PE/EA (3:1) to afford 400 mg (46.87%) of tert-butyl 2-(4-nitrophenyl)thiomorpholine-4-carboxylate as a yellow solid. ¹H NMR (300 MHz, DMSO-d₆) δ 8.33-8.17 (m, 2H), 7.77-7.66 (m, 2H), 7.71-7.54 (m, 1H), 6.92 (s, 1H), 4.34-4.09 (m, 3H), 3.20 (ddd, J=13.6, 10.1, 3.2 Hz, 1H), 2.88-2.72 (m, 1H), 2.77-2.65 (m, 1H), 2.45 (s, 1H), 1.74-1.57 (m, 1H), 1.57-1.45 (m, 1H), 1.43 (s, 2H), 1.40 (s, 8H), 1.29 (s, 2H), 1.25 (d, J=6.4 Hz, 3H), 1.15 (s, 1H), 0.99-0.76 (m, 2H).

### Step 5: Preparation of tert-butyl 2-(4-nitrophenyl)thiomorpholine-4-carboxylate 1,1-dioxide (Intermediate 33-e)

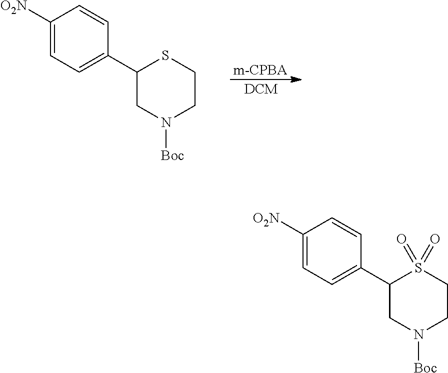

To a stirred solution of tert-butyl 2-(4-nitrophenyl)thiomorpholine-4-carboxylate (400 mg, 1.233 mmol, 1.00 equiv) in DCM(10 mL) was added m-CPBA (1063.91 mg, 6.165 mmol, 5 equiv). The resulting mixture was stirred at r.t. for overnight, added saturated Na₂SO₃ (aq.) (20 mL) and extracted twice with EtOAc (20 mL). The combined organic layers were washed twice with saturated NaHCO₃ (aq.) (20 mL) and brine (20 mL), dried over anhydrous Na₂SO₄, concentrated under reduced pressure to afford 420 mg of tert-butyl 2-(4-nitrophenyl)thiomorpholine-4-carboxylate 1,1-dioxide as a yellow solid. LCMS-APCI (POS.) m/z: 357 (M+H)⁺.

### Step 6: Preparation of tert-butyl 2-(4-aminophenyl)thiomorpholine-4-carboxylate 1,1-dioxide (Intermediate 33-f)

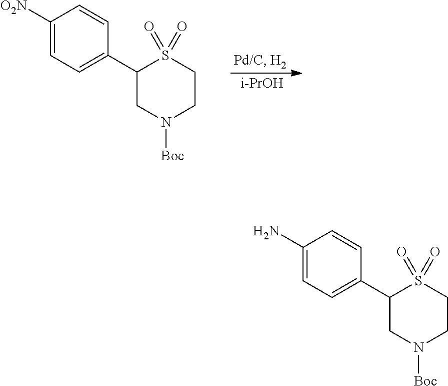 [SMILES: "i-PrOH" ; CC(C)(C)OC(=O)N1CCS(=O)(=O)C(c2ccc([N+](=O)[O-])cc2)C1 ; "Pd/C, H2" ; CC(C)(C)OC(=O)N1CCS(=O)(=O)C(c2ccc(N)cc2)C1]

To a stirred solution of tert-butyl 2-(4-nitrophenyl)-1,1-dioxo-1lambda6-thiomorpholine-4-carboxylate (420 mg, 1.178 mmol, 1.00 equiv) in i-PrOH(5 mL) was added Pd/C (10% Pd, 50% wet with water, 210 mg). The resulting mixture was stirred at r.t. for 2 h under H2, filtered to remove solids, and the filtration was concentrated under reduced pressure to afford 380 mg of tert-butyl 2-(4-aminophenyl)thiomorpholine-4-carboxylate 1,1-dioxide as a yellow solid. LCMS-APCI (POS.) m/z: 327 (M+H)⁺.

### Step 7: Preparation of tert-butyl 2-(4-(3-(4-methoxybenzyl)ureido)phenyl)thiomorpholine-4-carboxylate 1,1-dioxide (Intermediate 33-g)

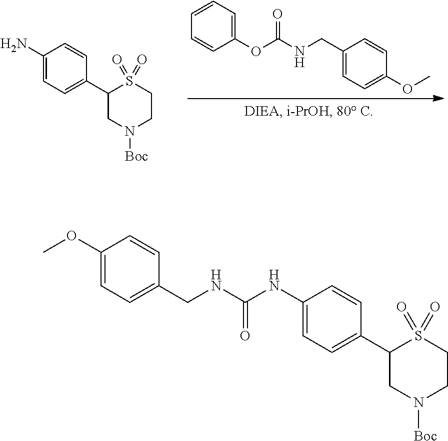 [SMILES: CC(C)(C)OC(=O)N1CCS(=O)(=O)C(c2ccc(N)cc2)C1 ; COc1ccc(CNC(=O)Oc2ccccc2)cc1 ; COc1ccc(CNC(=O)Nc2ccc(C3CN(C(=O)OC(C)(C)C)CCS3(=O)=O)cc2)cc1]

To a stirred solution of tert-butyl 2-(4-aminophenyl)thiomorpholine-4-carboxylate 1,1-dioxide (420 mg, 1.178 mmol, 1.00 equiv) in i-PrOH(5 mL) was added Pd/C (10% Pd, 50% wet with water, 210 mg). The resulting mixture was stirred at r.t. for 2 h under H₂, filtered to remove solids, and the filtration was concentrated under reduced pressure to afford 380 mg tert-butyl 2-(4-(3-(4-methoxybenzyl)ureido)phenyl)thiomorpholine-4-carboxylate 1,1-dioxide as a yellow solid. LCMS-APCI (POS.) m/z: 327 (M+H)⁺.

### Step 8: Preparation of 1-(4-(1,1-dioxidothiomorpholin-2-yl)phenyl)-3-(4-methoxybenzyl)urea (Intermediate 33)

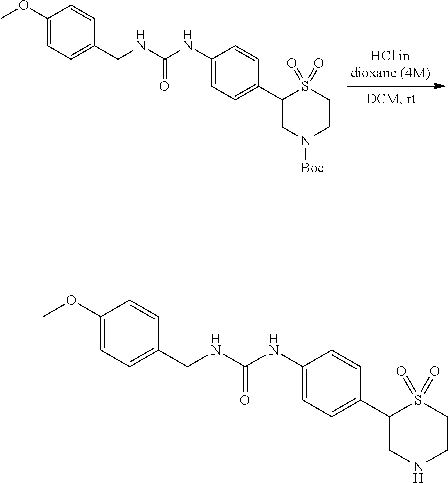 [SMILES: COc1ccc(CNC(=O)Nc2ccc(C3CN(C(=O)OC(C)(C)C)CCS3(=O)=O)cc2)cc1 ; COc1ccc(CNC(=O)Nc2ccc(C3CNCCS3(=O)=O)cc2)cc1]

To a stirred solution of tert-butyl 2-[4-({[(4-methoxyphenyl)methyl]carbamoyl}amino)phenyl]-1,1-dioxo-1lambda6-thiomorpholine-4-carboxylate (93 mg, 0.190 mmol, 1.00 equiv) in DCM (1 mL) was added HCl(gas)in 1,4-dioxane (0.5 mL, 4 mol/L). The resulting mixture was stirred at r.t. for 1 h, concentrated under reduced pressure, purified by C18 column chromatography, eluted with water(0.05% NH₄HCO₃)/ACN (4:1) to afford 45 mg (60.83%) of 3-[4-(1,1-dioxo-1lambda6-thiomorpholin-2-yl)phenyl]-1-[(4-methoxyphenyl)methyl]urea as a white solid. LCMS-APCI (POS.) m/z: 390 (M+H)⁺.

### Example HH

### Synthesis of N-methyl-N-(4-nitrobenzyl)acetamide (Intermediate 34.1-34.2)

### Preparation of N-methyl-N-(4-nitrobenzyl)acetamide (Intermediate 34)

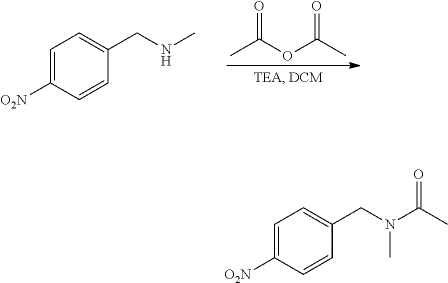

To a stirred mixture of methyl[(4-nitrophenyl)methyl]amine (500 mg, 3.009 mmol, 1.00 equiv) and TEA (456 mg, 4.506 mmol, 1.50 equiv) in DCM (4 mL) was added acetic anhydride (307 mg, 3.007 mmol, 1.00 equiv). The resulting mixture was stirred at r.t. for 2 h, and extracted twice with EtOAc (10 mL). The combined organic layers were washed twice with brine (10 mL), dried over anhydrous Na₂SO₄, concentrated under reduced pressure to afford 630 mg of N-methyl-N-[(4-nitrophenyl)methyl]acetamide as a brown solid LCMS-APCI (POS.) m/z: 208 (M+H)⁺.

Intermediate 30.2 was prepared in a similar manner as Intermediate 30.1

| Intermediate | Reagent | Structure, Name and Data |
|---|---|---|
| 34.2 | (4- nitrophenyl) methanamine | 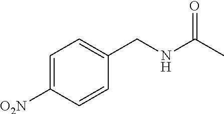 |
| | | N-(4-nitrobenzyl)acetamide. LCMS-APCI |
| | | (POS.) m/z: 194 (M + H)⁺. |

### Example II

### Synthesis of 1-(4-chlorobenzyl)-3-(4-(1-(methylsulfonyl)pyrrolidin-3-yl)phenyl)urea (Intermediate 35)

### Step 1: Preparation of tert-butyl 3-(((trifluoromethyl)sulfonyl)oxy)-2,5-dihydro-1H-pyrrole-1-carboxylate (Intermediate 35-a)

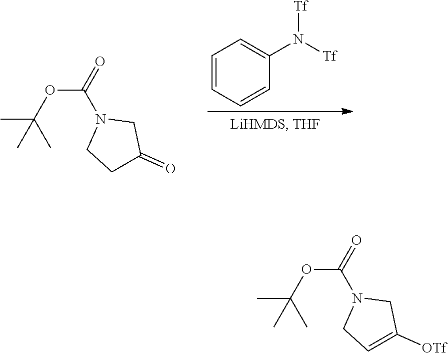

To a solution of 1-[tert-butoxy(hydroxy)methyl]pyrrolidin-3-one (5 g, 26.704 mmol, 1.00 equiv) in THE (50 mL) at −78° C. was added LiHMDS (53.8 mL, 1 mol/L in THF, 2 equiv) dropwise over a period of 30 min under nitrogen atmosphere. After stirred at −78° C. for 1 h under nitrogen atmosphere, the solution at −78° C. was added 1,1,1-trifluoro-N-phenyl-N-trifluoromethanesulfonylmethanesulfonamide (10.5 g, 29.392 mmol, 1.10 equiv) under nitrogen atmosphere. The resulting mixture was stirred at −78° C. for 2 h under nitrogen atmosphere. The product was no LCMS signal, determined by TLC. The reaction at 0° C. was quenched with water(50 mL), and extracted third with EtOAc (50 mL). The combined organic layers were washed twice with brine (50 mL), dried over anhydrous Na₂SO₄, concentrated under reduced pressure to afford 16 g of tert-butyl 3-(trifluoromethanesulfonyloxy)-2,5-dihydropyrrole-1-carboxylate (crude) as a brown oil.

### Step 2: Preparation of tert-butyl 3-(4-nitrophenyl)-2,5-dihydro-1H-pyrrole-1-carboxylate (Intermediate 35-b)

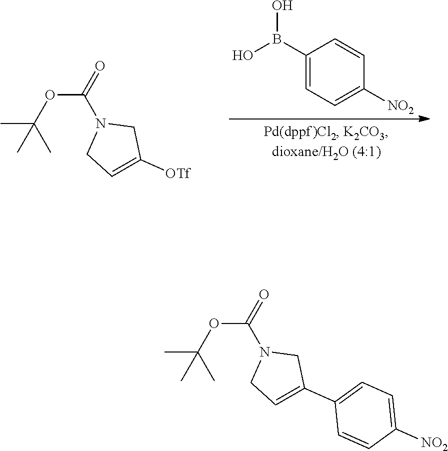 [SMILES: CC(C)(C)OC(=O)N1CC=C(OS(=O)(=O)C(F)(F)F)C1 ; O=[N+]([O-])c1ccc(B(O)O)cc1 ; CC(C)(C)OC(=O)N1CC=C(c2ccc([N+](=O)[O-])cc2)C1]

To a solution of tert-butyl 3-(trifluoromethanesulfonyloxy)-2,5-dihydropyrrole-1-carboxylate (8 g, 12.607 mmol, 1.00 equiv) and 4-nitrophenylboronic acid (2.5 g, 14.976 mmol, 1.19 equiv) in dioxane (40 mL) at r.t. were added Pd(dppf)Cl₂ (0.9 g, 1.230 mmol, 0.10 equiv) and a solution of K₂CO₃ (3.5 g, 25.325 mmol, 2.01 equiv) in H₂O (10 mL) under nitrogen atmosphere. The resulting mixture was stirred at 80° C. for overnight under nitrogen atmosphere. The reaction was determined by LCMS. The resulting mixture was cooled to r.t., concentrated under reduced pressure, and purified by silica gel column chromatography, eluted with PE/EA (6:1) to afford 2.4 g of tert-butyl 3-(4-nitrophenyl)-2,5-dihydropyrrole-1-carboxylate (65.57%) as a yellow solid. LCMS-APCI (POS.) m/z: 234 (M+H−56)⁺.

### Step 3: Preparation of tert-butyl 3-(4-aminophenyl)pyrrolidine-1-carboxylate (Intermediate 35-c)

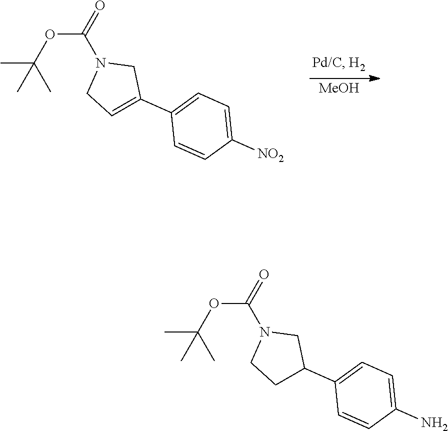 [SMILES: "MeOH" ; CC(C)(C)OC(=O)N1CC=C(c2ccc([N+](=O)[O-])cc2)C1 ; CC(C)(C)OC(=O)N1CCC(c2ccc(N)cc2)C1]

To a solution of tert-butyl 3-(4-nitrophenyl)-2,5-dihydropyrrole-1-carboxylate (2.3 g, 7.922 mmol, 1.00 equiv) in methanol (20 mL) at r.t. was added Pd/C (10% Pd, 50% wet with water, 2.3 g). The resulting mixture was stirred at r.t. for overnight under hydrogen atmosphere. The reaction was determined by LCMS. The resulting mixture was filtered to remove solids, concentrated under reduced pressure to afford 1.96 g of tert-butyl 3-(4-aminophenyl)pyrrolidine-1-carboxylate as a brown. LCMS-APCI (POS.) m/z: 206 (M+H−56)⁺.

### Step 4: Preparation of tert-butyl 3-(4-(3-(4-chlorobenzyl)ureido)phenyl)pyrrolidine-1-carboxylate (Intermediate 35-d)

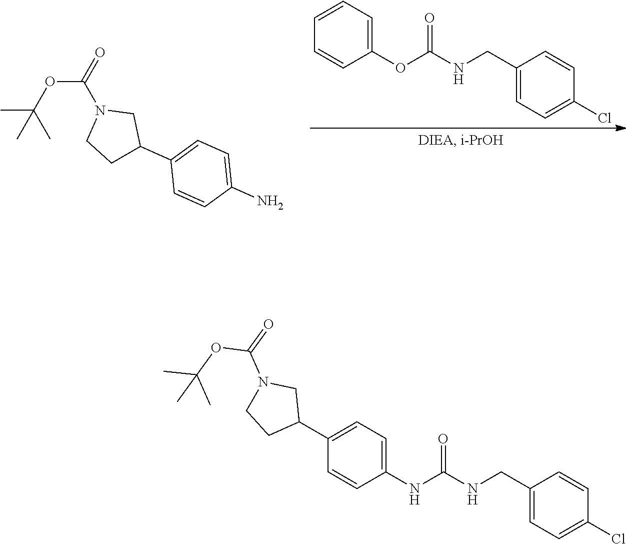 [SMILES: CC(C)(C)OC(=O)N1CCC(c2ccc(N)cc2)C1 ; O=C(NCc1ccc(Cl)cc1)Oc1ccccc1 ; CC(C)(C)OC(=O)N1CCC(c2ccc(NC(=O)NCc3ccc(Cl)cc3)cc2)C1]

To a solution of tert-butyl 3-(4-aminophenyl)pyrrolidine-1-carboxylate (600 mg, 2.287 mmol, 1.00 equiv) in i-PrOH (6 mL) at r.t. were added phenyl N-[(4-chlorophenyl)methyl]carbamate (896.6 mg, 3.426 mmol, 1.50 equiv) and DIEA (590.8 mg, 4.571 mmol, 2.00 equiv). The mixture was stirred at 80° C. for overnight, cooled to r.t., and purified by silica gel column chromatography, eluted with PE/EA (2:1) to afford 450 mg of tert-butyl 3-(4-(3-(4-chlorobenzyl)ureido)phenyl)pyrrolidine-1-carboxylate (45.76%) as a yellow semi-solid. LCMS-APCI (POS.) m/z: 347 (M+H−56)⁺.

### Step 5: Preparation of 1-(4-chlorobenzyl)-3-(4-(pyrrolidin-3-yl)phenyl)urea (Intermediate 35-e)

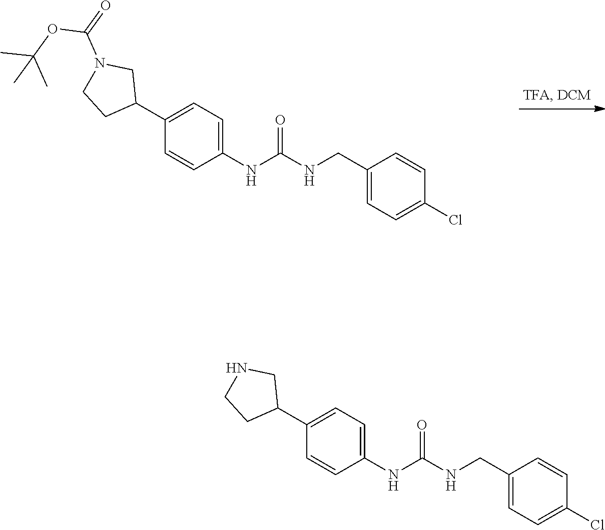 [SMILES: CC(C)(C)OC(=O)N1CCC(c2ccc(NC(=O)NCc3ccc(Cl)cc3)cc2)C1 ; O=C(NCc1ccc(Cl)cc1)Nc1ccc(C2CCNC2)cc1]

To a solution of tert-butyl 3-[4-({[(4-chlorophenyl)methyl]carbamoyl}amino)phenyl]pyrrolidine-1-carboxylate (400 mg, 0.930 mmol, 1.00 equiv) in DCM (4 mL) at r.t. was added TFA (1 mL). The resulting mixture was stirred at r.t. for 2 h. The reaction was determined by LCMS. The resulting mixture was adjusted pH to 10, and extracted third with CH₂Cl₂ (10 mL). The combined organic layers were washed twice with brine (10 mL), dried over anhydrous Na₂SO₄, concentrated under reduced pressure to afford 300 mg of 1-[(4-chlorophenyl)methyl]-3-[4-(pyrrolidin-3-yl)phenyl]urea as a brown semi-solid. LCMS-APCI (POS.) m/z: 330 (M+H)⁺.

### Step 6: Preparation of 1-(4-chlorobenzyl)-3-(4-(1-(methylsulfonyl)pyrrolidin-3-yl)phenyl)urea (Intermediate 35)

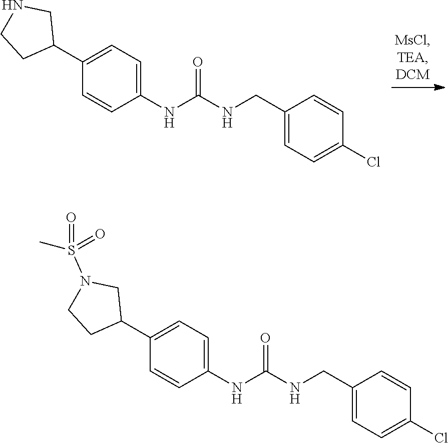 [SMILES: O=C(NCc1ccc(Cl)cc1)Nc1ccc(C2CCNC2)cc1 ; CS(=O)(=O)N1CCC(c2ccc(NC(=O)NCc3ccc(Cl)cc3)cc2)C1]

To a solution of 1-[(4-chlorophenyl)methyl]-3-[4-(pyrrolidin-3-yl)phenyl]urea (280 mg, 0.849 mmol, 1.00 equiv) and TEA (171.80 mg, 1.698 mmol, 2.00 equiv) in DCM (4 mL) at 0° C. was added MsCl (116.69 mg, 1.019 mmol, 1.2 equiv). The resulting mixture was stirred at r.t. for 4 h, and extracted third with CH₂Cl₂ (10 mL). The combined organic layers were washed twice with brine (10 mL), dried over anhydrous Na₂SO₄, concentrated under reduced pressure, purified by Prep-HPLC with the following conditions (2#SHIMADZU (HPLC-01)): Column, YMC-Actus Triart C18 ExRS, 30*150 mm, 5 μm; mobile phase, Water(10 mmol/L NH₄HCO₃) and ACN (30% ACN up to 60% in 8 min); Detector, UV254 nm, 210 nm to afford 230 mg of 1-[(4-chlorophenyl)methyl]-3-[4-(1-methanesulfonylpyrrolidin-3-yl)phenyl]urea (66.42%) as a brown solid. LCMS-APCI (POS.) m/z: 408 (M+H)⁺.

Intermediates 35.2 was prepared in a similar manner as Intermediate 35.1

| Intermediate | Reagents | Structure, Name and Data |
|---|---|---|
| 35.2 | phenyl N-[(4- methoxyphenyl)methyl]carbamate | 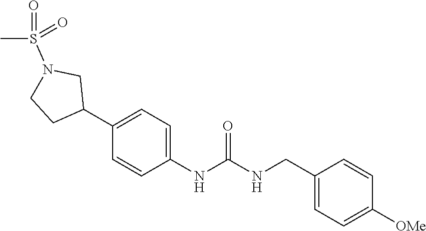 [SMILES: COc1ccc(CNC(=O)Nc2ccc(C3CCN(S(C)(=O)=O)C3)cc2)cc1] |
| | | 1-(4-methoxybenzyl)-3-(4-(1- |
| | | (methylsulfonyl)pyrrolidin-3- |
| | | yl)phenyl)urea. LC/MS (APCI) m/z: 404 |
| | | [M + H]. |

### Example JJ

### Synthesis of 4-(3-(4-chlorobenzyl)ureido)benzenesulfonyl chloride (Intermediate 36)

### Step 1: Preparation of 4-(3-(4-chlorobenzyl)ureido)benzenesulfonic acid (Intermediate 36-a)

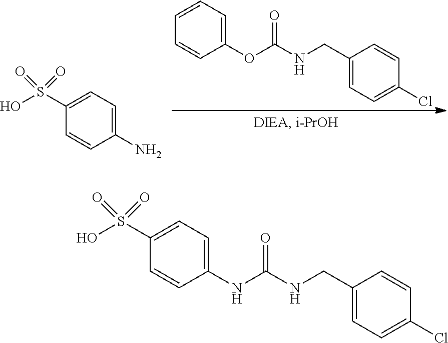 [SMILES: Nc1ccc(S(=O)(=O)O)cc1 ; O=C(NCc1ccc(Cl)cc1)Oc1ccccc1 ; O=C(NCc1ccc(Cl)cc1)Nc1ccc(S(=O)(=O)O)cc1]

To a stirred mixture of sulfanilic acid (2 g, 11.548 mmol, 1.00 equiv) and DIEA (14.91 g, 115.364 mmol, 9.99 equiv) in isopropyl alcohol (20 mL) was added phenyl N-[(4-chlorophenyl)methyl]carbamate (3.62 g, 13.832 mmol, 1.20 equiv). The resulting mixture was stirred at 80° C. for overnight, cooled to r.t., and purified by C₁₈ column chromatography, eluted with water(0.05% NH₄HCO₃)/ACN (20:1) to afford 3.7 g of 4-(3-(4-chlorobenzyl)ureido)benzenesulfonic acid as a brown solid. LC/MS (APCI) m/z: 341 [M+H].

### Step 2: Preparation of 4-(3-(4-chlorobenzyl)ureido)benzenesulfonyl chloride (Intermediate 36)

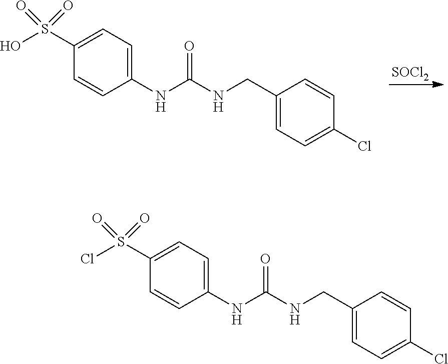 [SMILES: O=C(NCc1ccc(Cl)cc1)Nc1ccc(S(=O)(=O)O)cc1 ; "SOCl2" ; O=C(NCc1ccc(Cl)cc1)Nc1ccc(S(=O)(=O)Cl)cc1]

A solution of 4-({[(4-chlorophenyl)methyl]carbamoyl}amino)benzenesulfonic acid (3.5 g, 10.271 mmol, 1.00 equiv) in thionyl chloride (35 mL) was stirred at 60° C. for 30 min under nitrogen atmosphere. The mixture was cooled to r.t., and concentrated under reduced pressure to afford 3.8 g of 4-({[(4-chlorophenyl)methyl]carbamoyl}amino)benzenesulfonyl chloride as a yellow oil. LC/MS (APCI) m/z: 359 [M+H].

### Example KK

### Synthesis of 4-(2-oxaspiro[3.5]nonan-7-yl)aniline (Intermediate 37)

### Step 1: Preparation of 2-oxaspiro[3.5]non-6-en-7-yl trifluoromethanesulfonate (Intermediate 37-a)

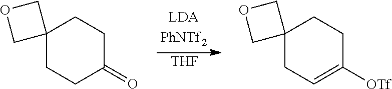 [SMILES: O=C1CCC2(CC1)COC2 ; O=S(=O)(OC1=CCC2(CC1)COC2)C(F)(F)F ; "THF"]

To a flame-dried flask was charged diisopropylamine (318 mg, 3.14 mmol, 1.1 equiv) and THE (6 mL). After cooling to −30° C., n-BuLi solution (1.32 mL, 3.13 mmol, 1.09 equiv) was added dropwise and the mixture was slowly warmed to −10° C. over 15 min. It was then cooled to −78° C. before a THF solution of 2-oxaspiro[3.5]nonan-7-one (400 mg, 2.85 mmol, 1.0 equiv) was added dropwise. The deprotonation was kept at −78° C. for 15 min and then taken out from bath for another 15 min. Then the flask was re-cooled to −78° C., a THE solution of PhNTf₂ (1.12 g, 3.14 mmol, 1.1 equiv) was added slowly and the reaction was again kept for 15 min at −78° C. and 1 h outside bath. Upon completion, half-saturated NH₄Cl solution was added and the aqueous phase was extracted with EtOAc (50 mL*3). The combined organic phase was dried (MgSO₄), filtered, and concentrated to yield the crude vinyl triflate, which was directly used in the next step. LCMS-ESI (POS.) m/z: 273.1 (M+H)⁺.

### Step 2: Preparation of 7-(4-nitrophenyl)-2-oxaspiro[3.5]non-6-ene (Intermediate 37-b)

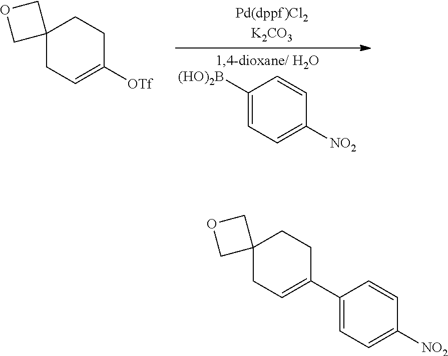 [SMILES: O=S(=O)(OC1=CCC2(CC1)COC2)C(F)(F)F ; O=[N+]([O-])c1ccc(B(O)O)cc1 ; O=[N+]([O-])c1ccc(C2=CCC3(CC2)COC3)cc1]

To a solution of 2-oxaspiro[3.5]non-6-en-7-yl trifluoromethanesulfonate (2.85 mmol, 1.0 equiv) and (4-nitrophenyl)boronic acid (714 mg, 4.28 mmol, 1.5 equiv) in dioxane/H₂O (10 mL, 3:1) was bubbled with N₂ for 10 min, followed by the addition of K₂CO₃ (794 mg, 5.71 mmol, 2.0 equiv) and Pd(dppf)Cl₂ (209 mg, 0.285 mmol, 0.1 equiv). The mixture was stirred at 75° C. for 15 h. Upon completion, half-saturated NH₄Cl solution was added and the aqueous phase was extracted with EtOAc (10 mL*2). The combined organic phase was dried (MgSO₄), filtered, concentrated, and purified by flash column chromatography (silica, hexanes/EtOAc=20/1->3/1) to yield the desired product as a yellowish waxy solid (512 mg, 73%). LCMS-ESI (POS.) m/z: 246.1 (M+H)⁺. ¹H NMR (400 MHz, Chloroform-d) δ 8.16 (d, J=8.9 Hz, 2H), 7.49 (d, J=8.8 Hz, 2H), 6.24 (tt, J=3.8, 1.6 Hz, 1H), 4.53 (d, J=5.8 Hz, 2H), 4.47 (d, J=5.8 Hz, 2H), 2.61 (dt, J=4.4, 2.5 Hz, 2H), 2.52 (tq, J=6.4, 2.1 Hz, 2H), 2.10 (t, J=6.3 Hz, 2H).

### Step 3: Preparation of 4-(2-oxaspiro[3.5]nonan-7-yl)aniline (Intermediate 37)

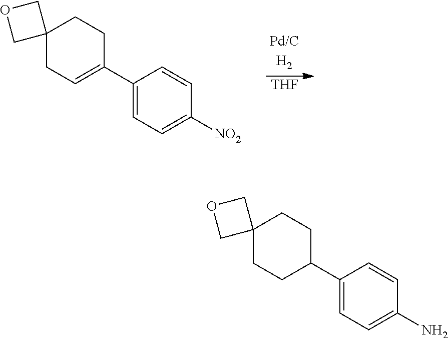 [SMILES: O=[N+]([O-])c1ccc(C2=CCC3(CC2)COC3)cc1 ; Nc1ccc(C2CCC3(CC2)COC3)cc1 ; "THF"]

To a solution 7-(4-nitrophenyl)-2-oxaspiro[3.5]non-6-ene (110 mg, 0.448, 1.0 equiv) in THE (6 mL) was added Pd/C (33 mg, 10% on wet basis, 30% mass equiv). H₂ was bubbled through for 3 min. The mixture was stirred at 23° C. for 14 h under H₂ atmosphere. Upon completion, solid was filtered off and the filtrate was concentrated to yield the aniline (90 mg, 93%). LCMS-ESI (POS.) m/z: 218.1 (M+H)⁺.

### Example LL

### Synthesis of 1-(4-chlorobenzyl)-3-(4-(2-hydroxyethyl)phenyl)urea (Intermediate 38)

### Step 1: Preparation of 1-(4-chlorobenzyl)-3-(4-(2-hydroxyethyl)phenyl)urea (Intermediate 38)

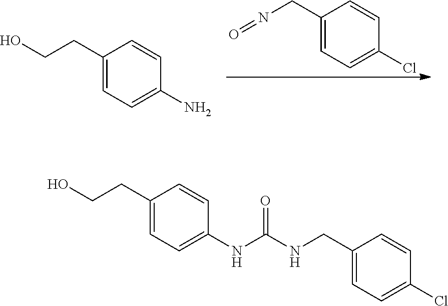 [SMILES: Nc1ccc(CCO)cc1 ; O=NCc1ccc(Cl)cc1 ; O=C(NCc1ccc(Cl)cc1)Nc1ccc(CCO)cc1]

To a solution of 2-(4-aminophenyl)ethan-1-ol (1.37 g, 10.0 mmol, 1.0 equiv) in CH₂Cl₂ (20 mL) was added p-chlorobenzyl isocyanate (1.70 g, 10.2 mmol, 1.02 equiv) slowly at 0° C. The mixture was then stirred vigorously at 23° C. for 1 h. Upon completion, precipitation was filtered and washed by cold CH₂Cl₂ (10 mL) and Et₂O (10 mL) to yield 1-(4-chlorobenzyl)-3-(4-(2-hydroxyethyl)phenyl)urea (2.8 g, 92%) as an off-white solid. LCMS-ESI (POS.) m/z: 305.10 (M+H)⁺. ¹H NMR (400 MHz, DMSO-d₆) δ 8.47 (s, 1H), 7.39 (d, J=8.4 Hz, 2H), 7.32 (d, J=8.4 Hz, 2H), 7.29 (d, J=8.4 Hz, 2H), 7.06 (d, J=8.2 Hz, 2H), 6.59 (t, J=6.0 Hz, 1H), 4.58 (s, 1H), 4.27 (d, J=6.0 Hz, 2H), 3.54 (t, J=7.2 Hz, 2H), 2.63 (t, J=7.2 Hz, 2H).

### Example MM

### Synthesis of 1-(4-(2-bromoethyl)phenyl)-3-(4-chlorobenzyl)urea (Intermediate 39)

### Preparation of 1-(4-(2-bromoethyl)phenyl)-3-(4-chlorobenzyl)urea (Intermediate 39)

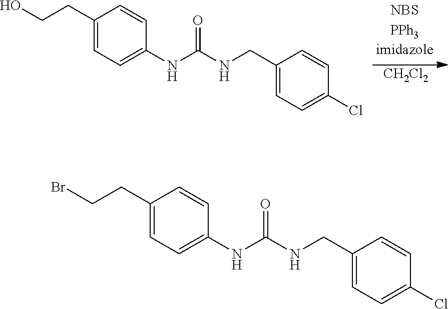

To a solution of 1-(4-chlorobenzyl)-3-(4-(2-hydroxyethyl)phenyl)urea (Intermediate 38, 500 mg, 1.64 mmol, 1.0 equiv) in THF/CH₂Cl₂ (20 mL, 1:1) was added PPh₃ (516 mg, 1.97 mmol, 1.2 equiv) and imidazole (167 mg, 2.46 mmol, 2.0 equiv). N-bromosuccinimide (350 mg, 1.97 mmol, 1.2 equiv) was then added at 0° C. The reaction was stirred at 23° C. for 1 h. Upon completion, a mixed solution of NaHCO₃ and Na₂S203 was added to quench the reaction. The aqueous phase was extracted by CH₂Cl₂ (5 mL). The combined organic phase was washed with brine, dried (MgSO₄), filtered, concentrated, and purified by column chromatography (silica, hexanes/EtOAc, 20:1->0:1) to yield 1-(4-(2-bromoethyl)phenyl)-3-(4-chlorobenzyl)urea (200 mg, 33%) as a white solid. LCMS-ESI (POS.) m/z: 367.00 (M+H)⁺. ¹H NMR (400 MHz, DMSO-d₆) δ 8.55 (s, 1H), 7.39 (d, J=8.5 Hz, 2H), 7.33 (d, J=5.6 Hz, 2H), 7.31 (d, J=5.6 Hz, 2H), 7.13 (d, J=8.5 Hz, 2H), 6.63 (t, J=6.0 Hz, 1H), 4.28 (d, J=6.0 Hz, 2H), 3.67 (t, J=7.3 Hz, 2H), 3.03 (t, J=7.3 Hz, 2H).

### Example 1

### Synthesis of ethyl 2-[4-({[(4-methoxyphenyl)methyl]amino}carbonylamino)phenyl]acetate (Compound 331)

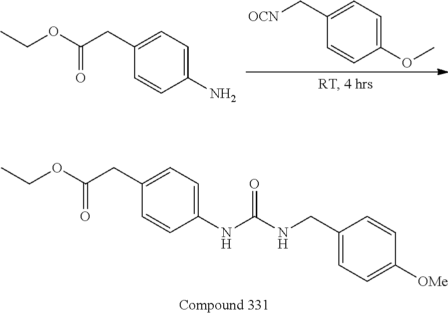

To a solution of ethyl 2-(4-aminophenyl)acetate (27.46 g, 153.2 mmol) in DCM (20 mL) at 20° C. was added 4-methoxy benzyl isocyanate (25.0 g, 153.2 mmol) dropwise. The resulting mixture was stirred at room temperature for 4 hours then methanol (10 mL) was added and cooled to 0° C. After 1 hour at 0° C. the slurry was filtered providing the desired product (26.7 g, 78.0 mmol, 50.9% yield) as an off-white solid. LCMS-APCI (POS.) m/z: 343.1 (M+H)+. 1H NMR (400 MHz, DMSO-d6) δ 8.50 (s, 1H), 7.38-7.30 (4, 2H), 7.27-7.19 (m, 2H), 7.15-7.07 (i, 2H), 6.94-6.85 (m, 2H), 6.52 (t, J=5.9 Hz, 1H), 4.22 (d, J=5.4 Hz, 2H), 4.06 (q, J=7.1 Hz, 2H), 3.73 (s, 3H), 3.55 (s, 2H), 1.17 (t, J=7.1 Hz, 3H).

Compounds in the following table were prepared in a similar manner as Compound 331, using the intermediates and reagents as listed.

| Ex # | Aniline | Isocyanate | Structure, Name and Data |
|---|---|---|---|
| 143 | ethyl 2- (4- amino phenyl) acetate | 4-chloro benzyl isocyanate | 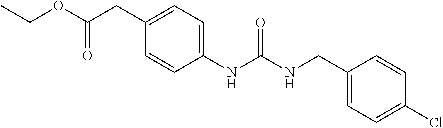 [SMILES: CCOC(=O)Cc1ccc(NC(=O)NCc2ccc(Cl)cc2)cc1] |
| | | | Compound 143: Ethyl 2-[4-({[(4- |
| | | | chlorophenyl)methyl]amino}carbonylamino) |
| | | | phenyl]acetate. LCMS-ESI (POS.) m/z: 347.0 (M + H)+. |
| | | | 1H NMR (400 MHz, DMSO-d6) δ 8.59 (s, 1H), 7.42- |
| | | | 7.26 (m, 6H), 7.16-7.05 (m, 2H), 6.65 (t, J = 6.0 |
| | | | Hz, 1H), 4.28 (d, J = 5.9 Hz, 2H), 4.06 (q, J = 7.1 Hz, |
| | | | 2H), 3.55 (s, 2H), 1.17 (t, J = 7.1 Hz, 3H). |
| 147 | tert- butyl 6- amino- 3,4- dihydro isoquinoline- 2(1H)- carboxylate | 4-chloro benzyl isocyanate | 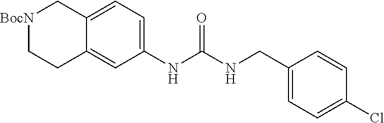 [SMILES: CC(C)(C)OC(=O)N1CCc2cc(NC(=O)NCc3ccc(Cl)cc3)ccc2C1] |
| | | | Compound 147: tert-butyl 6-({N-[(4- |
| | | | chlorophenyl)methyl]carbamoyl}amino)-1,2,3,4- |
| | | | tetrahydroisoquinoline-2-carboxylate. LCMS-ESI |
| | | | (POS.) m/z: 316.0 (M-Boc + H)+. 1H NMR (400 |
| | | | MHz, DMSO-d6) δ 8.51 (s, 1H), 7.40 (d, J = 8.4 Hz, |
| | | | 2H), 7.32 (d, J = 8.2 Hz, 2H), 7.27 (s, 1H), 7.22- |
| | | | 7.11 (m, 1H), 7.01 (d, J = 8.3 Hz, 1H), 6.63 (t, J = 6.0 |
| | | | Hz, 1H), 4.40 (s, 2H), 4.28 (d, J = 5.9 Hz, 2H), 3.52 |
| | | | (t, J = 5.9 Hz, 2H), 2.71 (t, J = 5.9 Hz, 2H), 1.43 (s, |
| | | | 9H). |
| 420 | Intermediate 13.1 | 4-methoxy benzyl isocyanate | 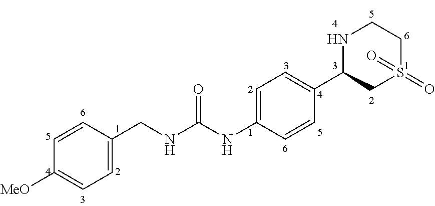 |
| | | | (R)-1-(4-(1,1-dioxidothiomorpholin-3-yl)phenyl)-3-(4-methox |
| | | | 1-(4-methoxybenzyl)-3-(4-((3-methyl-2- |
| | | | oxopyrrolidin-1-yl)methyl)phenyl)urea. LCMS- |
| | | | APCI (POS.) m/z: 368.1 (M + H)⁺. ¹H NMR (400 |
| | | | MHz, Methanol-d₄) δ 7.35 (d, J = 8.2 Hz, 2H), 7.24 |
| | | | (d, J = 8.3 Hz, 2H), 7.12 (d, J = 8.1 Hz, 2H), 6.86 (d, |
| | | | J = 8.3 Hz, 2H), 4.37 (d, J = 4.3 Hz, 2H), 4.32 (s, |
| | | | 2H), 3.78 (s, 3H), 3.23 (dd, J = 8.7, 5.1 Hz, 2H), 2.54 |
| | | | (h, J = 7.5 Hz, 1H), 2.24 (dq, J = 12.9, 6.3, 5.8 Hz, |
| | | | 1H), 1.61 (dq, J = 12.6, 8.3 Hz, 1H), 1.20 (d, J = 7.2 |
| | | | Hz, 3H). |
| 421 | Intermediate 13.2 | 4-methoxy benzyl isocyanate | 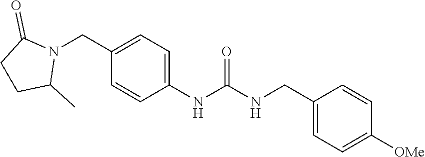 [SMILES: COc1ccc(CNC(=O)Nc2ccc(CN3C(=O)CCC3C)cc2)cc1] |
| | | | 1-(4-methoxybenzyl)-3-(4-((2-methyl-5- |
| | | | oxopyrrolidin-1-yl)methyl)phenyl)urea. LCMS- |
| | | | APCI (POS.) m/z = 368.1 (M + H)⁺. ¹H NMR (400 |
| | | | MHz, Methanol-d₄) δ 7.40-7.31 (m, 2H), 7.26 (d, J = |
| | | | 8.2 Hz, 2H), 7.17 (d, J = 8.1 Hz, 2H), 6.94-6.85 |
| | | | (m, 2H), 4.80 (d, J = 15.0 Hz, 1H), 4.32 (s, 2H), 4.07 |
| | | | (d, J = 15.0 Hz, 1H), 3.79 (s, 3H), 3.61 (h, J = 6.2 Hz, |
| | | | 1H), 2.60-2.32 (m, 2H), 2.29-2.14 (m, 1H), 1.64 |
| | | | (ddd, J = 13.4, 10.6, 6.5 Hz, 1H), 1.20 (dd, J = 6.3, |
| | | | 1.2 Hz, 3H). |
| 361 | N-(4- amino benzyl)- 1- phenyl methane sulfonamide | 4-chloro benzyl isocyanate | 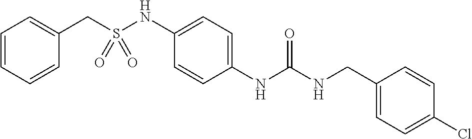 [SMILES: O=C(NCc1ccc(Cl)cc1)Nc1ccc(NS(=O)(=O)Cc2ccccc2)cc1] |
| | | | N-(4-(3-(4-chlorobenzyl)ureido)phenyl)-1- |
| | | | phenylmethanesulfonamide. LCMS-APCI (POS.) |
| | | | m/z: 430.2 (M + H)⁺. ¹H NMR (400 MHz, DMSO-d₆) δ |
| | | | 9.55 (d, J = 2.8 Hz, 1H), 8.59 (d, J = 2.8 Hz, 1H), |
| | | | 7.36 (qd, J = 10.8, 9.9, 7.1 Hz, 9H), 7.28 (q, J = 4.4, |
| | | | 3.8 Hz, 2H), 7.09 (dd, J = 8.9, 2.8 Hz, 2H), 6.63 (q, J = |
| | | | 5.2 Hz, 1H), 4.36 (s, 2H), 4.29 (s, 2H). |
| 362 | N-(4- amino benzyl)- 1- phenyl methane sulfonamide | 4-methoxy benzyl isocyanate | 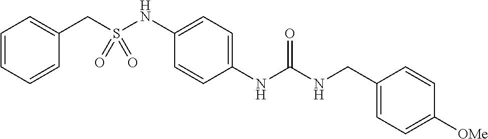 [SMILES: COc1ccc(CNC(=O)Nc2ccc(NS(=O)(=O)Cc3ccccc3)cc2)cc1] |
| | | | N-(4-(3-(4-methoxybenzyl)ureido)phenyl)-1- |
| | | | phenylmethanesulfonamide. LCMS-APCI (POS.) |
| | | | m/z: 426.2 (M + H)⁺. ¹H NMR (400 MHz, DMSO-d₆) δ |
| | | | 9.53 (s, 1H), 8.48 (s, 1H), 7.40-7.33 (m, 5H), 7.31- |
| | | | 7.20 (m, 4H), 7.08 (d, J = 8.0 Hz, 2H), 6.90 (d, J = |
| | | | 8.1 Hz, 2H), 6.49 (s, 1H), 4.35 (s, 2H), 4.22 (s, 2H), |
| | | | 3.73 (s, 3H). |
| 368 | Intermediate 13.3 | 4-chloro benzyl isocyanate | 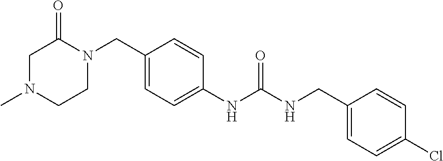 [SMILES: CN1CCN(Cc2ccc(NC(=O)NCc3ccc(Cl)cc3)cc2)C(=O)C1] |
| | | | 1-(4-chlorobenzyl)-3-(4-((4-methyl-2-oxopiperazin- |
| | | | 1-yl)methyl)phenyl)urea. LCMS-APCI (POS.) m/z: |
| | | | 387.1 (M + H)⁺. ¹H NMR (400 MHz, Chloroform-d) δ |
| | | | 7.30 (d, J = 2.2 Hz, 1H), 7.28-7.21 (m, 4H), 7.17 |
| | | | (d, J = 8.5 Hz, 2H), 7.07 (d, J = 8.4 Hz, 2H), 5.90 (t, |
| | | | J = 5.6 Hz, 1H), 4.49 (s, 2H), 4.37 (d, J = 5.8 Hz, |
| | | | 2H), 3.30 (t, J = 5.4 Hz, 2H), 3.14 (s, 2H), 2.67 (t, J = |
| | | | 5.4 Hz, 2H), 2.36 (s, 3H). |
| 509 | Intermediate 15.1 | 4-chloro benzyl isocyanate | 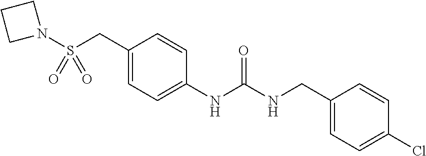 [SMILES: O=C(NCc1ccc(Cl)cc1)Nc1ccc(CS(=O)(=O)N2CCC2)cc1] |
| | | | 1-(4-((azetidin-1-ylsulfonyl)methyl)phenyl)-3-(4- |
| | | | chlorobenzyl)urea. LCMS-APCI (POS.) m/z: 394.1 |
| | | | (M + H)⁺. ¹H NMR (DMSO-d₆) δ: 8.69 (s, 1H), 7.45- |
| | | | 7.37 (m, 4H), 7.37-7.25 (m, 4H), 6.69 (t, J = 6.0 Hz, 1H), |
| | | | 4.38 (s, 2H), 4.29 (d, J = 5.9 Hz, 2H), 3.80 (t, J = 7.7 Hz, |
| | | | 4H), 2.14 (p, J = 7.7 Hz, 2H). |
| 510 | Intermediate 15.2 | 4-chloro benzyl isocyanate | 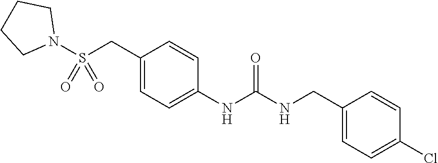 [SMILES: O=C(NCc1ccc(Cl)cc1)Nc1ccc(CS(=O)(=O)N2CCCC2)cc1] |
| | | | 1-(4-chlorobenzyl)-3-(4-((pyrrolidin-1- |
| | | | ylsulfonyl)methyl)phenyl)urea. LCMS-APCI (POS.) |
| | | | m/z: 408.1 (M + H)⁺. ¹H NMR (DMSO-d₆) δ: 8.68 (s, 1H), |
| | | | 7.43-7.27 (m, 6H), 7.26 (d, J = 8.5 Hz, 2H), 6.69 (t, J = |
| | | | 6.1 Hz, 1H), 4.32 (s, 2H), 4.28 (d, J = 6.0 Hz, 2H), 3.19 (s, |
| | | | 1H), 3.16-3.08 (m, 3H), 1.78 (td, J = 7.3, 6.7, 4.0 Hz, |
| | | | 4H). |
| 511 | Intermediate 15.3 | 4-chloro benzyl isocyanate | 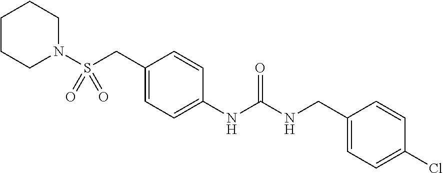 [SMILES: O=C(NCc1ccc(Cl)cc1)Nc1ccc(CS(=O)(=O)N2CCCCC2)cc1] |
| | | | 1-(4-chlorobenzyl)-3-(4-((piperidin-1- |
| | | | ylsulfonyl)methyl)phenyl)urea. LCMS-APCI (POS.) |
| | | | m/z: 422.1 (M + H)⁺. ¹H NMR (DMSO-d₆) δ: 8.68 (s, 1H), |
| | | | 7.44-7.37 (m, 4H), 7.37-7.28 (m, 2H), 7.28-7.21 (m, |
| | | | 2H), 6.69 (t, J = 6.0 Hz, 1H), 4.28 (d, J = 5.9 Hz, 2H), 4.25 |
| | | | (s, 2H), 3.05 (d, J = 5.5 Hz, 4H), 1.49-1.43 (m, 6H). |
| 537 | Intermediate 15.4 | 4-chloro benzyl isocyanate | 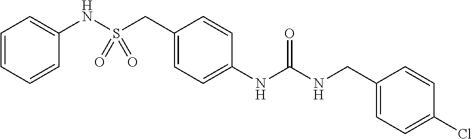 [SMILES: O=C(NCc1ccc(Cl)cc1)Nc1ccc(CS(=O)(=O)Nc2ccccc2)cc1] |
| | | | 1-(4-(3-(4-chlorobenzyl)ureido)phenyl)-N- |
| | | | phenylmethanesulfonamide. LCMS-APCI (POS.) |
| | | | m/z: 430.2 (M + H)⁺. ¹H NMR (DMSO-d₆) δ: 9.76 (s, 1H), |
| | | | 8.70 (s, 1H), 7.45-7.29 (m, 9H), 7.23-7.16 (m, 2H), 7.12- |
| | | | 7.07 (m, 3H), 6.70 (t, J = 5.9 Hz, 1H), 5.76 (s, 3H), 4.33 (s, 2H), |
| | | | 4.28 (d, J = 5.9 Hz, 2H). |
| | Intermediate 13.3 | 4-chloro benzyl isocyanate | 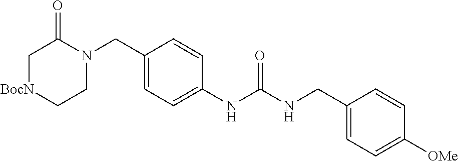 [SMILES: COc1ccc(CNC(=O)Nc2ccc(CN3CCN(C(=O)OC(C)(C)C)CC3=O)cc2)cc1] |
| | | | tert-butyl 4-(4-(3-(4-chlorobenzyl)ureido)benzyl)- |
| | | | 3-oxopiperazine-1-carboxylate. LCMS-APCI |
| | | | (POS.) m/z: 413 (M + H-56)⁺. |
| 385 | Intermediate 13.5 | 4-methoxy benzyl isocyanate | 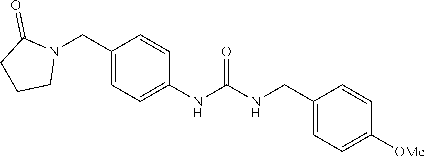 [SMILES: COc1ccc(CNC(=O)Nc2ccc(CN3CCCC3=O)cc2)cc1] |
| | | | 1-(4-methoxybenzyl)-3-(4-((2-oxopyrrolidin-1- |
| | | | yl)methyl)phenyl)urea. LCMS-APCI (POS.) m/z: |
| | | | 354 (M + H)⁺. ¹H NMR (DMSO-d₆) δ: 8.50 (s, 1H), |
| | | | 7.35 (d, J = 8.1 Hz, 2H), 7.22 (d, J = 8.1 Hz, 2H), |
| | | | 7.07 (d, J = 8.1 Hz, 2H), 6.88 (d, J = 8.1 Hz, 2H), |
| | | | 6.50 (s, 1H), 4.29-4.17 (m, 4H), 3.72 (s, 3H), 3.18 |
| | | | (t, J = 7.1 Hz, 2H), 2.26 (t, J = 7.9 Hz, 2H), 1.90 (d, J = |
| | | | 9.2 Hz, 2H). |
| | Intermediate 13.6 | 4-chloro benzyl isocyanate | 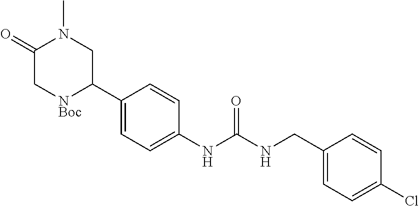 [SMILES: CN1CC(c2ccc(NC(=O)NCc3ccc(Cl)cc3)cc2)N(C(=O)OC(C)(C)C)CC1=O] |
| | | | tert-butyl 4-(4-(3-(4-chlorobenzyl)ureido)benzyl)- |
| | | | 3-oxopiperazine-1-carboxylate. LCMS-APCI |
| | | | (POS.) m/z: 473 (M + H)⁺. |
| | Intermediate 13.7 | 4-chloro benzyl isocyanate | 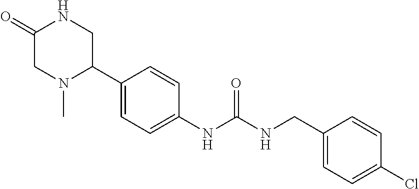 [SMILES: CN1CC(=O)NCC1c1ccc(NC(=O)NCc2ccc(Cl)cc2)cc1] |
| | | | 1-(4-chlorobenzyl)-3-(4-(1-methyl-5-oxopiperazin- |
| | | | 2-yl)phenyl)urea. LCMS-APCI (POS.) m/z: 373 |
| | | | (M + H)⁺. |
| | Intermediate 13.8 | 4-chloro benzyl isocyanate | 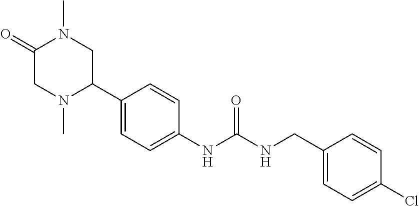 [SMILES: CN1CC(c2ccc(NC(=O)NCc3ccc(Cl)cc3)cc2)N(C)CC1=O] |
| | | | 1-(4-chlorobenzyl)-3-(4-(1,4-dimethyl-5- |
| | | | oxopiperazin-2-yl)phenyl)urea. LCMS-APCI |
| | | | (POS.) m/z: 387 (M + H)⁺. |
| | Intermediate 13.9 | 4-chloro benzyl isocyanate | 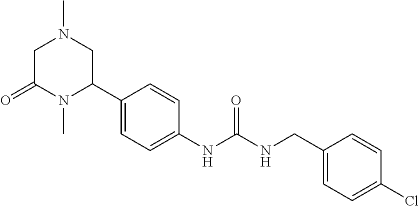 [SMILES: CN1CC(=O)N(C)C(c2ccc(NC(=O)NCc3ccc(Cl)cc3)cc2)C1] |
| | | | 1-(4-chlorobenzyl)-3-(4-(1,4-dimethyl-6- |
| | | | oxopiperazin-2-yl)phenyl)urea. LCMS-APCI |
| | | | (POS.) m/z: 387 (M + H)⁺. |
| | Intermediate 13.7 | 4-methoxy benzyl isocyanate | 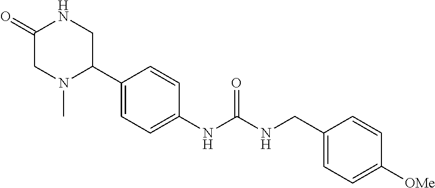 [SMILES: COc1ccc(CNC(=O)Nc2ccc(C3CNC(=O)CN3C)cc2)cc1] |
| | | | 1-(4-methoxybenzyl)-3-(4-(1-methyl-5- |
| | | | oxopiperazin-2-yl)phenyl)urea. LCMS-APCI |
| | | | (POS.) m/z: 369 (M + H)⁺. |
| | Intermediate 13.7 | 4-fluoro benzyl isocyanate | 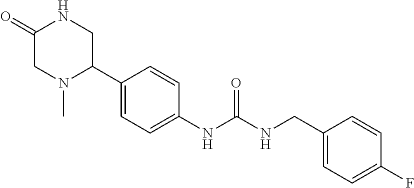 [SMILES: CN1CC(=O)NCC1c1ccc(NC(=O)NCc2ccc(F)cc2)cc1] |
| | | | 1-(4-fluorobenzyl)-3-(4-(1-methyl-5-oxopiperazin- |
| | | | 2-yl)phenyl)urea. LCMS-APCI (POS.) m/z: 357 |
| | | | (M + H)⁺. |
| 402 | Intermediate 13.10 | 4-chloro benzyl isocyanate | 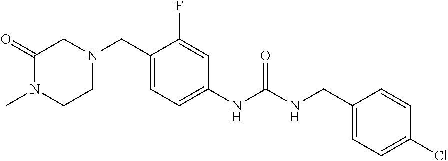 [SMILES: CN1CCN(Cc2ccc(NC(=O)NCc3ccc(Cl)cc3)cc2F)CC1=O] |
| | | | 1-(4-chlorobenzyl)-3-(3-fluoro-4-((4-methyl-3- |
| | | | oxopiperazin-1-yl)methyl)phenyl)urea. LCMS- |
| | | | APCI (POS.) m/z: 405 (M + H)⁺. ¹H NMR (300 |
| | | | MHz, MeOD-d₄) δ 7.57 (dd, J = 12.8, 2.1 Hz, 1H), 7.40 |
| | | | (t, J = 8.4 Hz, 1H), 7.34 (d, J = 1.4 Hz, 4H), 7.18 (dd, J = |
| | | | 8.4, 2.3 Hz, 1H), 4.40 (s, 2H), 4.27 (s, 2H), 3.73 (s, 2H), |
| | | | 3.58 (t, J = 5.6 Hz, 2H), 3.44 (d, J = 5.7 Hz, 2H), 3.01 (s, |
| | | | 3H). |
| 394 | Intermediate 13.11 | 4-chloro benzyl isocyanate | 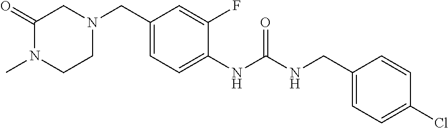 [SMILES: CN1CCN(Cc2ccc(NC(=O)NCc3ccc(Cl)cc3)c(F)c2)CC1=O] |
| | | | 1-(4-chlorobenzyl)-3-(2-fluoro-4-((4-methyl-3- |
| | | | oxopiperazin-1-yl)methyl)phenyl)urea. LCMS- |
| | | | APCI (POS.) m/z: 405 (M + H)⁺. ¹H NMR (DMSO- |
| | | | d₆) δ: 8.38 (s, 1H), 8.04 (t, J = 8.5 Hz, 1H), 7.44- |
| | | | 7.35 (m, 2H), 7.35-7.27 (m, 2H), 7.17-6.97 (m, |
| | | | 3H), 4.29 (d, J = 5.8 Hz, 2H), 3.45 (s, 2H), 3.24 (d, J = |
| | | | 11.0 Hz, 2H), 2.92 (s, 2H), 2.80 (s, 3H), 2.60 (t, J = |
| | | | 5.5 Hz, 2H). |
| 395 | Intermediate 13.12 | 4-chloro benzyl isocyanate | 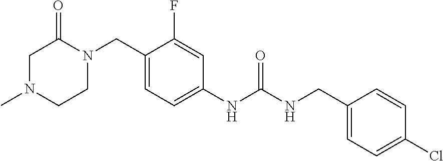 [SMILES: CN1CCN(Cc2ccc(NC(=O)NCc3ccc(Cl)cc3)cc2F)C(=O)C1] |
| | | | 1-(4-chlorobenzyl)-3-(3-fluoro-4-((4-methyl-2- |
| | | | oxopiperazin-1-yl)methyl)phenyl)urea. LCMS- |
| | | | APCI (POS.) m/z: 405 (M + H)⁺. ¹H NMR (DMSO- |
| | | | d₆) δ: 8.87 (s, 1H), 7.48 (dd, J = 13.1, 2.0 Hz, 1H), |
| | | | 7.43-7.36 (m, 2H), 7.32 (d, J = 8.4 Hz, 2H), 7.11 (t, |
| | | | J = 8.5 Hz, 1H), 7.01 (dd, J = 8.3, 2.1 Hz, 1H), 6.74 |
| | | | (t, J = 6.0 Hz, 1H), 4.47 (s, 2H), 4.28 (d, J = 5.9 Hz, |
| | | | 2H), 3.18 (t, J = 5.5 Hz, 2H), 2.98 (s, 2H), 2.56 (t, J = |
| | | | 5.5 Hz, 2H), 2.20 (s, 3H). |
| 401 | Intermediate 13.13 | 4-chloro benzyl isocyanate | 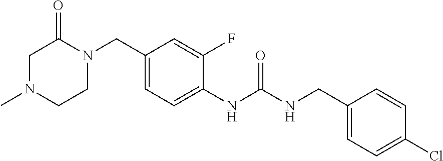 [SMILES: CN1CCN(Cc2ccc(NC(=O)NCc3ccc(Cl)cc3)c(F)c2)C(=O)C1] |
| | | | 1-(4-chlorobenzyl)-3-(2-fluoro-4-((4-methyl-2- |
| | | | oxopiperazin-1-yl)methyl)phenyl)urea. LCMS- |
| | | | APCI (POS.) m/z: 405 (M + H)⁺. ¹H NMR (DMSO- |
| | | | d₆) δ: 8.41 (s, 1H), 8.05 (t, J = 8.5 Hz, 1H), 7.45- |
| | | | 7.37 (m, 2H), 7.36-7.29 (m, 2H), 7.11-7.00 (m, |
| | | | 2H), 7.00-6.93 (m, 1H), 4.44 (s, 2H), 4.30 (d, J = |
| | | | 5.9 Hz, 2H), 3.18 (dd, J = 6.2, 4.8 Hz, 2H), 3.00 (s, |
| | | | 2H), 2.56 (dd, J = 6.3, 4.7 Hz, 2H), 2.21 (s, 3H). |
| | Intermediate 13.14 | 4-chloro benzyl isocyanate | 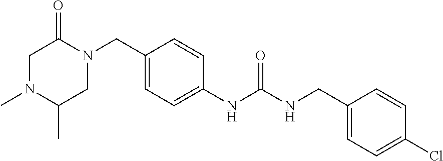 [SMILES: CC1CN(Cc2ccc(NC(=O)NCc3ccc(Cl)cc3)cc2)C(=O)CN1C] |
| | | | 1-(4-chlorobenzyl)-3-(4-((4,5-dimethyl-2- |
| | | | oxopiperazin-1-yl)methyl)phenyl)urea. LCMS- |
| | | | APCI (POS.) m/z: 401 (M + H)⁺. |
| | Intermediate 13.15 | 4-chloro benzyl isocyanate | 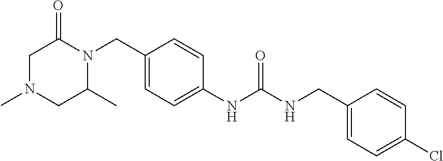 [SMILES: CC1CN(C)CC(=O)N1Cc1ccc(NC(=O)NCc2ccc(Cl)cc2)cc1] |
| | | | 1-(4-chlorobenzyl)-3-(4-((2,4-dimethyl-6- |
| | | | oxopiperazin-1-yl)methyl)phenyl)urea. LCMS- |
| | | | APCI (POS.) m/z: 401 (M + H)⁺. |
| | Intermediate 13.14 | 4-methoxy benzyl isocyanate | 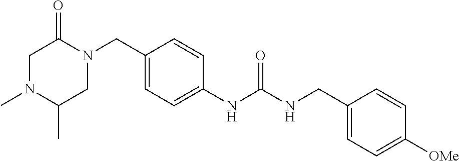 [SMILES: COc1ccc(CNC(=O)Nc2ccc(CN3CC(C)N(C)CC3=O)cc2)cc1] |
| | | | 1-(4-((4,5-dimethyl-2-oxopiperazin-1- |
| | | | yl)methyl)phenyl)-3-(4-methoxybenzyl)urea. |
| | | | LCMS-APCI (POS.) m/z: 397 (M + H)⁺. |
| | Intermediate 13.15 | 4-methoxy benzyl isocyanate | 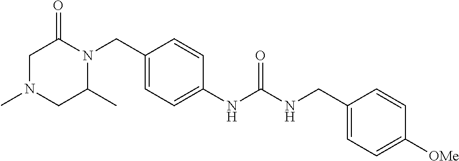 [SMILES: COc1ccc(CNC(=O)Nc2ccc(CN3C(=O)CN(C)CC3C)cc2)cc1] |
| | | | 1-(4-((2,4-dimethyl-6-oxopiperazin-1- |
| | | | yl)methyl)phenyl)-3-(4-methoxybenzyl)urea. |
| | | | LCMS-APCI (POS.) m/z: 397 (M + H)⁺. |
| | Intermediate 13.16 | 4-chloro benzyl isocyanate | 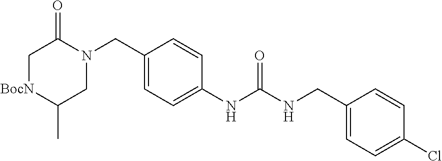 [SMILES: CC1CN(Cc2ccc(NC(=O)NCc3ccc(Cl)cc3)cc2)C(=O)CN1C(=O)OC(C)(C)C] |
| | | | tert-butyl 4-(4-(3-(4-chlorobenzyl)ureido)benzyl)- |
| | | | 2-methyl-5-oxopiperazine-1-carboxylate. LCMS- |
| | | | APCI (POS.) m/z: 487 (M + H)⁺. |
| | Intermediate 13.17 | 4-chloro benzyl isocyanate | 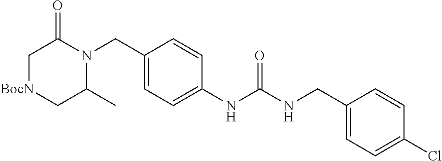 [SMILES: CC1CN(C(=O)OC(C)(C)C)CC(=O)N1Cc1ccc(NC(=O)NCc2ccc(Cl)cc2)cc1] |
| | | | tert-butyl 4-(4-(3-(4-chlorobenzyl)ureido)benzyl)- |
| | | | 3-methyl-5-oxopiperazine-1-carboxylate. LCMS- |
| | | | APCI (POS.) m/z: 431 (M + H-56)⁺. |
| | Intermediate 13.18 | 4-chloro benzyl isocyanate | 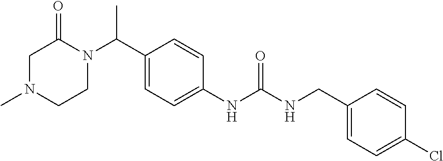 [SMILES: CC(c1ccc(NC(=O)NCc2ccc(Cl)cc2)cc1)N1CCN(C)CC1=O] |
| | | | 1-(4-chlorobenzyl)-3-(4-(1-(4-methyl-2- |
| | | | oxopiperazin-1-yl)ethyl)phenyl)urea. LCMS-APCI |
| | | | (POS.) m/z: 401 (M + H)⁺. |
| 414 | Intermediate 13.19 | 4-chloro benzyl isocyanate | 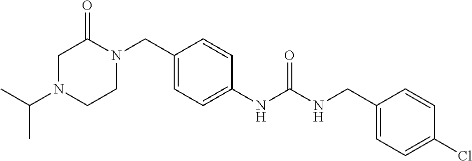 [SMILES: CC(C)N1CCN(Cc2ccc(NC(=O)NCc3ccc(Cl)cc3)cc2)C(=O)C1] |
| | | | 1-(4-chlorobenzyl)-3-(4-((4-isopropyl-2- |
| | | | oxopiperazin-1-yl)methyl)phenyl)urea. LCMS- |
| | | | APCI (POS.) m/z: 415 (M + H)⁺. ¹H NMR (400 |
| | | | MHz, Chloroform-d) δ 8.60 (s, 1H), 7.43-7.28 (m, |
| | | | 6H), 7.14-7.06 (m, 2H), 6.64 (t, J = 6.0 Hz, 1H), |
| | | | 4.41 (s, 2H), 4.28 (d, J = 6.0 Hz, 2H), 3.11 (d, J = 4.8 |
| | | | Hz, 4H), 2.66 (p, J = 6.4 Hz, 1H), 2.64-2.57 (m, |
| | | | 2H), 0.96 (d, J = 6.5 Hz, 6H). |
| | Intermediate 13.20 | 4-chloro benzyl isocyanate | 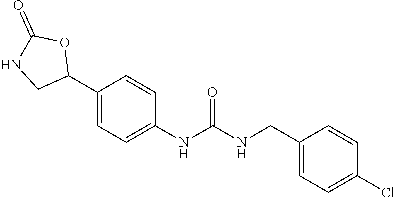 [SMILES: O=C(NCc1ccc(Cl)cc1)Nc1ccc(C2CNC(=O)O2)cc1] |
| | | | 1-(4-chlorobenzyl)-3-(4-(2-oxooxazolidin-5- |
| | | | yl)phenyl)urea. LCMS-APCI (POS.) m/z: 346 |
| | | | (M + H)⁺. |
| | Intermediate 13.21 | 4-chloro benzyl isocyanate | 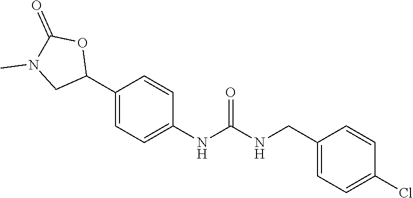 [SMILES: CN1CC(c2ccc(NC(=O)NCc3ccc(Cl)cc3)cc2)OC1=O] |
| | | | 1-(4-chlorobenzyl)-3-(4-(3-methyl-2-oxooxazolidin- |
| | | | 5-yl)phenyl)urea. LCMS-APCI (POS.) m/z: 360 |
| | | | (M + H)⁺. |
| | Intermediate 13.21 | 4-methoxy benzyl isocyanate | 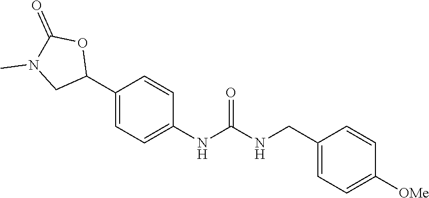 [SMILES: COc1ccc(CNC(=O)Nc2ccc(C3CN(C)C(=O)O3)cc2)cc1] |
| | | | 1-(4-methoxybenzyl)-3-(4-(3-methyl-2- |
| | | | oxooxazolidin-5-yl)phenyl)urea. LCMS-APCI |
| | | | (POS.) m/z: 356 (M + H)⁺. |
| | Intermediate 13.21 | 4-fluoro benzyl isocyanate | 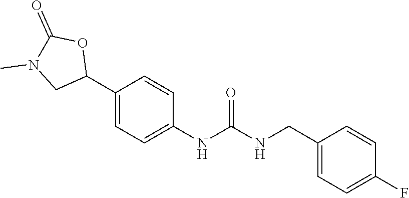 [SMILES: CN1CC(c2ccc(NC(=O)NCc3ccc(F)cc3)cc2)OC1=O] |
| | | | 1-(4-fluorobenzyl)-3-(4-(3-methyl-2-oxooxazolidin- |
| | | | 5-yl)phenyl)urea. LCMS-APCI (POS.) m/z: 344 |
| | | | (M + H)⁺. |
| | Intermediate 13.22 | 4-chloro benzyl isocyanate | 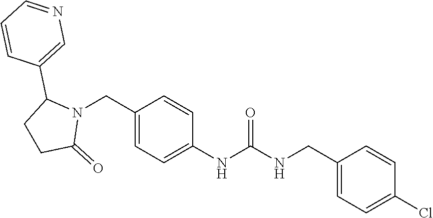 [SMILES: O=C(NCc1ccc(Cl)cc1)Nc1ccc(CN2C(=O)CCC2c2cccnc2)cc1] |
| | | | 1-(4-chlorobenzyl)-3-(4-((2-oxo-5-(pyridin-3- |
| | | | yl)pyrrolidin-1-yl)methyl)phenyl)urea. LCMS- |
| | | | APCI (POS.) m/z: 435 (M + H)⁺. |
| | Intermediate 13.23 | 4-chloro benzyl isocyanate | 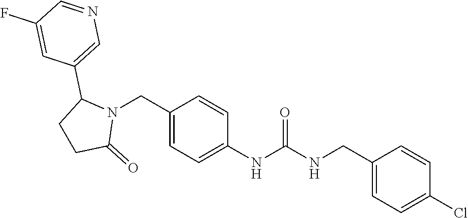 [SMILES: O=C(NCc1ccc(Cl)cc1)Nc1ccc(CN2C(=O)CCC2c2cncc(F)c2)cc1] |
| | | | 1-(4-chlorobenzyl)-3-(4-((2-(5-fluoropyridin-3-yl)- |
| | | | 5-oxopyrrolidin-1-yl)methyl)phenyl)urea. LCMS- |
| | | | APCI (POS.) m/z: 435 (M + H)⁺. |
| 450 | Intermediate 13.24 | 4-methoxy benzyl isocyanate | 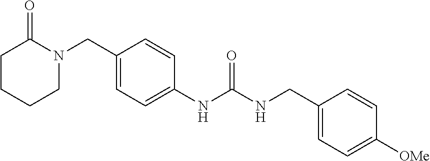 [SMILES: COc1ccc(CNC(=O)Nc2ccc(CN3CCCCC3=O)cc2)cc1] |
| | | | 1-(4-methoxybenzyl)-3-(4-((2-oxopiperidin-1- |
| | | | yl)methyl)phenyl)urea. LCMS-APCI (POS.) m/z: |
| | | | 387 (M + H)⁺. ¹H NMR (DMSO-d₆) δ: 8.48 (s, 1H), |
| | | | 7.40-7.28 (m, 2H), 7.28-7.16 (m, 2H), 7.13-7.01 |
| | | | (m, 2H), 6.96-6.84 (m, 2H), 6.49 (t, J = 5.9 Hz, |
| | | | 1H), 4.40 (s, 2H), 4.21 (d, J = 5.8 Hz, 2H), 3.73 (s, |
| | | | 3H), 3.18-3.09 (m, 2H), 2.35-2.26 (m, 2H), 1.69 |
| | | | (p, J = 3.1 Hz, 4H). |
| | Intermediate 13.25 | 4-chloro benzyl isocyanate | 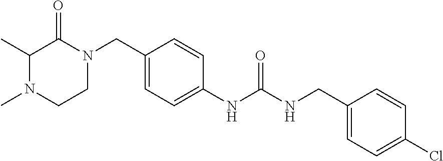 [SMILES: CC1C(=O)N(Cc2ccc(NC(=O)NCc3ccc(Cl)cc3)cc2)CCN1C] |
| | | | 1-(4-chlorobenzyl)-3-(4-((3,4-dimethyl-2- |
| | | | oxopiperazin-1-yl)methyl)phenyl)urea. LCMS- |
| | | | APCI (POS.) m/z: 401 (M + H)⁺. |
| | Intermediate 13.26 | 4-chloro benzyl isocyanate | 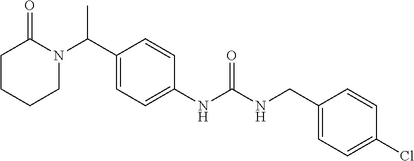 [SMILES: CC(c1ccc(NC(=O)NCc2ccc(Cl)cc2)cc1)N1CCCCC1=O] |
| | | | 1-(4-chlorobenzyl)-3-(4-(1-(2-oxopiperidin-1- |
| | | | yl)ethyl)phenyl)urea. LCMS-APCI (POS.) m/z: 386 |
| | | | (M + H)⁺. |
| | Intermediate 13.27 | 4-chloro benzyl isocyanate | 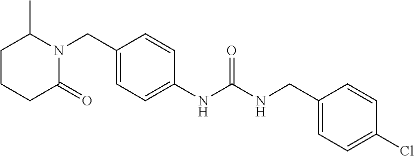 [SMILES: CC1CCCC(=O)N1Cc1ccc(NC(=O)NCc2ccc(Cl)cc2)cc1] |
| | | | 1-(4-chlorobenzyl)-3-(4-((2-methyl-6-oxopiperidin- |
| | | | 1-yl)methyl)phenyl)urea. LCMS-APCI (POS.) m/z: |
| | | | 386 (M + H)⁺. |
| | Intermediate 13.28 | 4-chloro benzyl isocyanate | 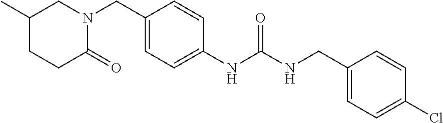 [SMILES: CC1CCC(=O)N(Cc2ccc(NC(=O)NCc3ccc(Cl)cc3)cc2)C1] |
| | | | 1-(4-chlorobenzyl)-3-(4-((5-methyl-2-oxopiperidin- |
| | | | 1-yl)methyl)phenyl)urea. LCMS-APCI (POS.) m/z: |
| | | | 386 (M + H)⁺. |
| | Intermediate 13.29 | 4-chloro benzyl isocyanate | 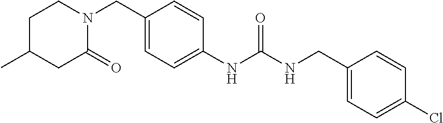 [SMILES: CC1CCN(Cc2ccc(NC(=O)NCc3ccc(Cl)cc3)cc2)C(=O)C1] |
| | | | 1-(4-chlorobenzyl)-3-(4-((4-methyl-2-oxopiperidin- |
| | | | 1-yl)methyl)phenyl)urea. LCMS-APCI (POS.) m/z: |
| | | | 386 (M + H)⁺. |
| | Intermediate 13.29 | 4-methoxy benzyl isocyanate | 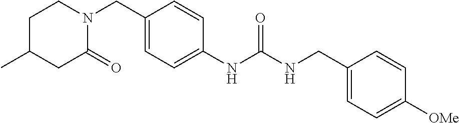 [SMILES: COc1ccc(CNC(=O)Nc2ccc(CN3CCC(C)CC3=O)cc2)cc1] |
| | | | 1-(4-methoxybenzyl)-3-(4-((4-methyl-2- |
| | | | oxopiperidin-1-yl)methyl)phenyl)urea. LCMS- |
| | | | APCI (POS.) m/z: 382 (M + H)⁺. |
| | Intermediate 13.30 | 4-chloro benzyl isocyanate | 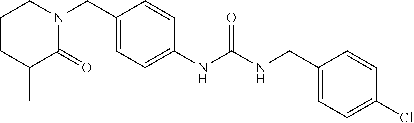 [SMILES: CC1CCCN(Cc2ccc(NC(=O)NCc3ccc(Cl)cc3)cc2)C1=O] |
| | | | 1-(4-chlorobenzyl)-3-(4-((3-methyl-2-oxopiperidin- |
| | | | 1-yl)methyl)phenyl)urea. LCMS-APCI (POS.) m/z: |
| | | | 386 (M + H)⁺. |
| | Intermediate 13.30 | 4- methoxy benzyl isocyanate | 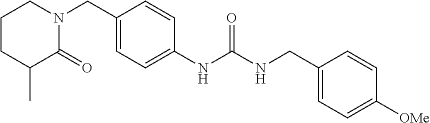 [SMILES: COc1ccc(CNC(=O)Nc2ccc(CN3CCCC(C)C3=O)cc2)cc1] |
| | | | 1-(4-methoxybenzyl)-3-(4-((3-methyl-2- |
| | | | oxopiperidin-1-yl)methyl)phenyl)urea. LCMS- |
| | | | APCI (POS.) m/z: 382 (M + H)⁺. |
| 492 | Intermediate 13.31 | 4- methoxy benzyl isocyanate | 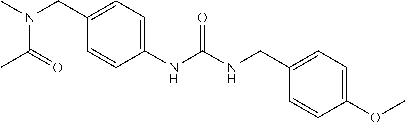 |
| | | | N-(4-(3-(4-methoxybenzyl)ureido)benzyl)-N- |
| | | | methylacetamide. LCMS-APCI (POS.) m/z: 342 |
| | | | (M + H)⁺. ¹H NMR (DMSO-d₆) δ: 8.49 (d, J = 14.0 Hz, |
| | | | 1H), 7.49-7.29 (m, 2H), 7.28-7.01 (m, 4H), 6.94-6.82 |
| | | | (m, 2H), 6.49 (q, J = 5.7 Hz, 1H), 4.41 (d, J = 16.2 Hz, |
| | | | 2H), 4.21 (d, J = 5.8 Hz, 2H), 3.73 (s, 3H), 2.81 (d, J = |
| | | | 33.0 Hz, 3H), 2.04 (d, J = 2.6 Hz, 3H). |
| 493 | Intermediate 13.31 | 4- chlorobenzyl isocyanate | 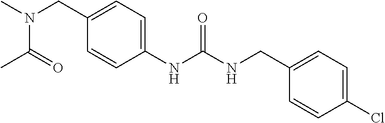 [SMILES: CC(=O)N(C)Cc1ccc(NC(=O)NCc2ccc(Cl)cc2)cc1] |
| | | | N-(4-(3-(4-chlorobenzyl)ureido)benzyl)-N- |
| | | | methylacetamide. LCMS-APCI (POS.) m/z: 346 |
| | | | (M + H)⁺. ¹H NMR (DMSO-d₆) δ: 8.59 (d, J = 14.4 Hz, |
| | | | 1H), 7.53-7.22 (m, 6H), 7.14-6.94 (m, 2H), 6.63 (d, J = |
| | | | 6.1 Hz, 1H), 4.41 (d, J = 16.2 Hz, 2H), 4.28 (d, J = 6.0 Hz, |
| | | | 2H), 2.81 (dd, J = 33.1, 2.0 Hz, 3H), 2.04 (d, J = 2.2 Hz, |
| | | | 3H). |
| 472 | Intermediate 13.32 | 4- chlorobenzyl isocyanate | 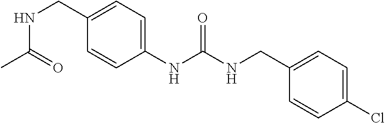 [SMILES: CC(=O)NCc1ccc(NC(=O)NCc2ccc(Cl)cc2)cc1] |
| | | | N-(4-(3-(4-chlorobenzyl)ureido)benzyl)acetamide. |
| | | | LCMS-APCI (POS.) m/z: 331 (M + H)⁺. ¹H NMR |
| | | | (DMSO-d₆) δ: 8.54 (s, 1H), 8.32-8.12 (m, 1H), 7.50- |
| | | | 7.37 (m, 2H), 7.36-7.25 (m, 4H), 7.15-7.04 (m, 2H), |
| | | | 6.61 (t, J = 6.0 Hz, 1H), 4.27 (d, J = 6.0 Hz, 2H), 4.15 (d, J = |
| | | | 5.9 Hz, 2H), 1.85 (s, 3H). |
| 491 | 4- (phenyl sulfonyl) aniline | 4- methoxy benzyl isocyanate | 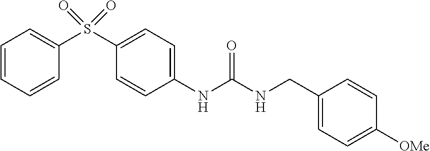 [SMILES: COc1ccc(CNC(=O)Nc2ccc(S(=O)(=O)c3ccccc3)cc2)cc1] |
| | | | 1-(4-methoxybenzyl)-3-(4- |
| | | | (phenylsulfonyl)phenyl)urea. LCMS-APCI (POS.) |
| | | | m/z: 397.1 (M + H)⁺. ¹H NMR (DMSO-d₆) δ: 9.09 (s, |
| | | | 1H), 7.94-7.84 (m, 2H), 7.84-7.74 (m, 2H), 7.71- |
| | | | 7.51 (m, 5H), 7.27-7.15 (m, 2H), 6.92-6.82 (m, |
| | | | 2H), 6.74 (t, J = 5.9 Hz, 1H), 4.22 (d, J = 5.8 Hz, 2H), |
| | | | 3.72 (s, 3H) |
| 390 | Intermediate 13.33 | 4- chlorobenzyl isocyanate | 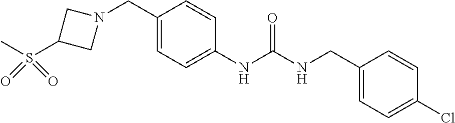 [SMILES: CS(=O)(=O)C1CN(Cc2ccc(NC(=O)NCc3ccc(Cl)cc3)cc2)C1] |
| | | | 1-(4-chlorobenzyl)-3-(4-((3- |
| | | | (methylsulfonyl)azetidin-1-yl)methyl)phenyl)urea. |
| | | | LCMS-APCI (POS.) m/z: 408.1 (M + H)⁺. ¹H NMR |
| | | | (DMSO-d₆) δ: 8.56 (s, 1H), 7.44-7.36 (m, 2H), 7.36- |
| | | | 7.28 (m, 4H), 7.16-7.07 (m, 2H), 6.62 (t, J = 6.0 |
| | | | Hz, 1H), 4.28 (d, J = 5.9 Hz, 2H), 4.20-4.04 (m, |
| | | | 1H), 3.46 (t, J = 8.1 Hz, 4H), 3.39-3.25 (m, 2H), |
| | | | 2.95 (s, 3H). |
| 449 | 1-(4- amino benzyl) piperidin- 2-one | 4- chlorobenzyl isocyanate | 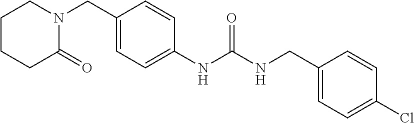 [SMILES: O=C(NCc1ccc(Cl)cc1)Nc1ccc(CN2CCCCC2=O)cc1] |
| | | | 1-(4-chlorobenzyl)-3-(4-((2-oxopiperidin-1- |
| | | | yl)methyl)phenyl)urea. LCMS-APCI (POS.) m/z: |
| | | | 372.10 (M + H)⁺. ¹H NMR (400 MHz, DMSO-d₆) δ 8.58 |
| | | | (s, 1 H), 7.39 (d, J = 8.1 Hz, 2 H), 7.35 (d, J = 8.1 Hz, 2 |
| | | | H), 7.32 (d, J = 8.2 Hz, 2 H), 7.09 (d, J = 8.1 Hz, 2 H), |
| | | | 6.63 (t, J = 6.1 Hz, 1 H), 4.40 (s, 2 H), 4.28 (d, J = 5.9 Hz, |
| | | | 2 H), 3.20-3.07 (m, 2 H), 2.34-2.23 (m, 2 H), 1.74-1.62 |
| | | | (m, 4 H). |
| 433 | 4- (pyridin- 4- ylmethyl) aniline | 4-fluoro benzyl isocyanate | 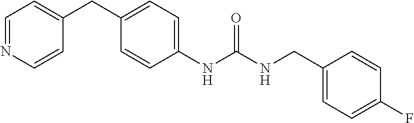 [SMILES: O=C(NCc1ccc(F)cc1)Nc1ccc(Cc2ccncc2)cc1] |
| | | | N-[(4-fluorophenyl)methyl]{[4-(4- |
| | | | pyridylmethyl)phenyl]amino}carboxamide. LCMS-ESI |
| | | | (POS.) m/z: 336.1 (M + H)⁺. ¹H NMR (400 MHz, DMSO- |
| | | | d₆) δ 8.52 (s, 1 H), 8.44 (d, J = 4.5 Hz, 2 H), 7.39-7.27 (m, |
| | | | 4 H), 7.21 (d, J = 4.6 Hz, 2 H), 7.15 (t, J = 9.0 Hz, 2 H), |
| | | | 7.10 (d, J = 7.8 Hz, 2 H), 6.59 (t, J = 6.0 Hz, 1 H), 4.27 (d, |
| | | | J = 5.8 Hz, 2 H), 3.87 (s, 2 H). |
| 431 | 4- (pyridin- 2- ylmethyl) aniline | 4-chloro benzyl isocyanate | 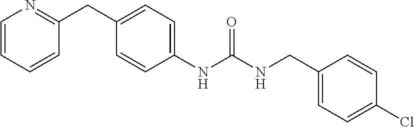 [SMILES: O=C(NCc1ccc(Cl)cc1)Nc1ccc(Cc2ccccn2)cc1] |
| | | | N-[(4-chlorophenyl)methyl]{[4-(2- |
| | | | pyridylmethyl)phenyl]amino}carboxamide. LCMS-ESI |
| | | | (POS.) m/z: 351.90 (M + H)⁺. ¹H NMR (400 MHz, DMSO- |
| | | | d₆) δ 8.53 (s, 1 H), 8.47 (s, 1 H), 8.44-8.35 (m, 1 H), 7.59 |
| | | | (d, J = 7.8 Hz, 1 H), 7.42-7.36 (m, 2 H), 7.35-7.26 (m, 5 |
| | | | H), 7.10 (d, J = 8.0 Hz, 2 H), 6.61 (s, 1 H), 4.27 (d, J = 5.9 |
| | | | Hz, 2 H), 3.88 (s, 2 H). |
| 432 | 4- (pyridin- 3- ylmethyl) aniline | 4-fluoro benzyl isocyanate | 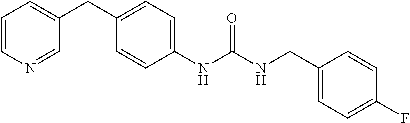 [SMILES: O=C(NCc1ccc(F)cc1)Nc1ccc(Cc2cccnc2)cc1] |
| | | | N-[(4-fluorophenyl)methyl]{[4-(3- |
| | | | pyridylmethyl)phenyl]amino}carboxamide. LCMS-ESI |
| | | | (POS.) m/z: 336.10 (M + H)⁺. ¹H NMR (400 MHz, DMSO- |
| | | | d₆) δ 8.49 (d, J = 10.1 Hz, 2 H), 8.40 (d, J = 4.6 Hz, 1 H), |
| | | | 7.59 (dd, J = 7.8, 2.1 Hz, 1 H), 7.36-7.26 (m, 5 H), 7.19- |
| | | | 7.07 (m, 4 H), 6.58 (s, 1 H), 4.26 (d, J = 5.8 Hz, 2 H), 3.88 |
| | | | (s, 2 H). |
| 435 | 4- (tetra hydro- 2H- pyran- 4- yl)aniline | 4-chloro benzyl isocyanate | 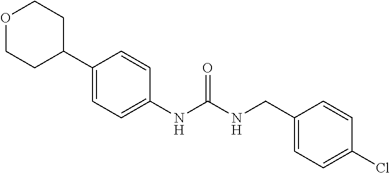 [SMILES: O=C(NCc1ccc(Cl)cc1)Nc1ccc(C2CCOCC2)cc1] |
| | | | 1-(4-chlorobenzyl)-3-(4-(tetrahydro-2H-pyran-4- |
| | | | yl)phenyl)urea. LCMS-ESI (POS.) m/z: 345.10 (M + H)⁺. |
| | | | ¹H NMR (400 MHz, DMSO-d₆) δ 8.50 (s, 1 H), 7.39 (d, J = |
| | | | 8.1 Hz, 2 H), 7.32 (d, J = 7.6 Hz, 4 H), 7.10 (d, J = 8.0 |
| | | | Hz, 2 H), 6.60 (t, J = 6.0 Hz, 1 H), 4.28 (d, J = 5.9 Hz, 2 |
| | | | H), 3.93 (dd, J = 10.6, 3.7 Hz, 2 H), 3.41 (td, J = 11.0, 3.2 |
| | | | Hz, 2 H), 2.67 (tt, J = 10.5, 4.9 Hz, 1 H), 1.71-1.53 (m, 4 |
| | | | H). |
| 440 | 4- (tetra hydro- 2H- pyran- 4- yl)aniline | 4-chloro benzyl isocyanate | 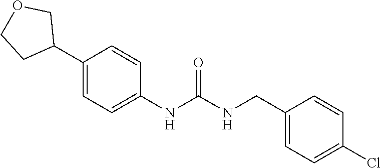 [SMILES: O=C(NCc1ccc(Cl)cc1)Nc1ccc(C2CCOC2)cc1] |
| | | | 1-[(4-chlorophenyl)methyl]-3-[4-(oxolan-3- |
| | | | yl)phenyl]urea. LCMS-ESI (POS.) m/z: 331.10 |
| | | | (M + H)⁺. ¹H NMR (400 MHz, DMSO-d₆) δ 8.53 (s, 1 |
| | | | H), 7.39 (d, J = 8.1 Hz, 2 H), 7.36-7.28 (m, 4 H), |
| | | | 7.13 (d, J = 8.1 Hz, 2 H), 6.61 (t, J = 6.1 Hz, 1 H), |
| | | | 4.28 (d, J = 5.9 Hz, 2 H), 3.99 (t, J = 7.8 Hz, 1 H), |
| | | | 3.96-3.88 (m, 1 H), 3.78 (q, J = 7.8 Hz, 1 H), 3.48 (t, |
| | | | J = 8.0 Hz, 1 H), 3.28 (p, J = 7.8 Hz, 1 H), 2.27 (dd, J = |
| | | | 12.5, 7.3 Hz, 1 H), 1.93-1.81 (m, 1 H). |
| 441 | 4-((1H- imidazol- 1- yl)methyl) aniline | 4-chloro benzyl isocyanate | 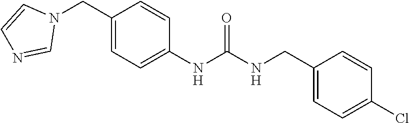 [SMILES: O=C(NCc1ccc(Cl)cc1)Nc1ccc(Cn2ccnc2)cc1] |
| | | | 1-(4-((1H-imidazol-1-yl)methyl)phenyl)-3-(4- |
| | | | chlorobenzyl)urea. LCMS-ESI (POS.) m/z: 341.10 |
| | | | (M + H)⁺. ¹H NMR (400 MHz, DMSO-d₆) δ 8.64 (s, 1 H), |
| | | | 7.70 (s, 1 H), 7.42-7.35 (m, 4 H), 7.31 (d, J = 8.1 Hz, 2 |
| | | | H), 7.18-7.12 (m, 3 H), 6.88 (s, 1 H), 6.65 (s, 1 H), 5.08 |
| | | | (s, 2 H), 4.27 (d, J = 5.9 Hz, 2 H). |
| 442 | 4-((2- methyl- 1H- imidazol- 1- yl)methyl) aniline | 4-chloro benzyl isocyanate | 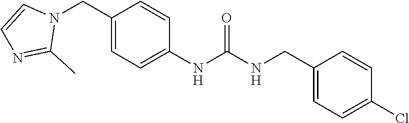 [SMILES: Cc1nccn1Cc1ccc(NC(=O)NCc2ccc(Cl)cc2)cc1] |
| | | | 1-(4-chlorobenzyl)-3-(4-((2-methyl-1H-imidazol-1- |
| | | | yl)methyl)phenyl)urea. LCMS-ESI (POS.) m/z: 355.10 |
| | | | (M + H)⁺. ¹H NMR (400 MHz, DMSO-d₆) δ 8.63 (s, 1 H), |
| | | | 7.43-7.35 (m, 4 H), 7.31 (d, J = 8.0 Hz, 2 H), 7.08 (s, 1 |
| | | | H), 7.04 (d, J = 8.1 Hz, 2 H), 6.73 (s, 1 H), 6.65 (s, 1 H), |
| | | | 5.02 (s, 2 H), 4.27 (d, J = 5.9 Hz, 2 H), 2.23 (s, 3 H). |
| 444 | 1-(4- amino benzyl) pyridin- 2(1H)- one | 4-chloro benzyl isocyanate | 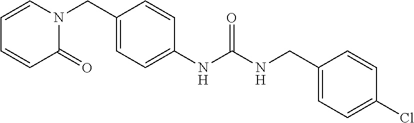 [SMILES: O=C(NCc1ccc(Cl)cc1)Nc1ccc(Cn2ccccc2=O)cc1] |
| | | | 1-(4-chlorobenzyl)-3-(4-((2-oxopyridin-1(2H)- |
| | | | yl)methyl)phenyl)urea. LCMS-ESI (POS.) m/z: 368.10 |
| | | | (M + H)⁺. ¹H NMR (400 MHz, DMSO-d₆) δ 8.62 (s, 1 H), |
| | | | 7.74 (d, J = 6.8 Hz, 1 H), 7.44-7.33 (m, 5 H), 7.31 (d, J = |
| | | | 8.0 Hz, 2 H), 7.19 (d, J = 8.1 Hz, 2 H), 6.64 (t, J = 6.1 Hz, |
| | | | 1 H), 6.40 (d, J = 9.1 Hz, 1 H), 6.21 (t, J = 6.7 Hz, 1 H), |
| | | | 4.99 (s, 2 H), 4.27 (d, J = 5.9 Hz, 2 H). |
| 546 | 4-((1H- 1,2,4- triazol- 1- yl)methyl) aniline | 4- benzyl isocyanate | 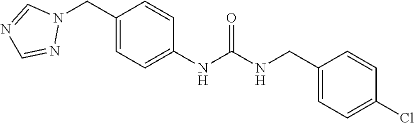 [SMILES: O=C(NCc1ccc(Cl)cc1)Nc1ccc(Cn2cncn2)cc1] |
| | | | 1-(4-((1H-1,2,4-triazol-1-yl)methyl)phenyl)-3-(4- |
| | | | chlorobenzyl)urea. LCMS-ESI (POS.) m/z: 342.05 |
| | | | (M + H)⁺. ¹H NMR (400 MHz, DMSO-d₆) δ 8.65 (s, 1 H), |
| | | | 8.60 (s, 1 H), 7.95 (s, 1 H), 7.43-7.35 (m, 4 H), 7.31 (d, J = |
| | | | 8.1 Hz, 2 H), 7.17 (d, J = 8.1 Hz, 2 H), 6.66 (t, J = 6.1 |
| | | | Hz, 1 H), 5.30 (s, 2 H), 4.27 (d, J = 6.0 Hz, 2 H). |
| 445 | 4-((3- cyclo propyl- 1,2,4- oxadiazol- 5- yl)methyl) aniline | 4-chloro benzyl isocyanate | 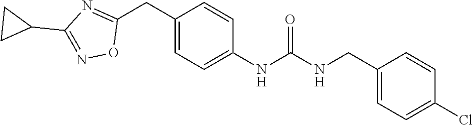 [SMILES: O=C(NCc1ccc(Cl)cc1)Nc1ccc(Cc2nc(C3CC3)no2)cc1] |
| | | | 1-(4-chlorobenzyl)-3-(4-((3-cyclopropyl-1,2,4- |
| | | | oxadiazol-5-yl)methyl)phenyl)urea. LCMS-ESI (POS.) |
| | | | m/z: 383.10 (M + H)⁺. ¹H NMR (400 MHz, DMSO-d₆) δ |
| | | | 8.62 (s, 1 H), 7.42-7.34 (m, 4 H), 7.31 (d, J = 8.1 Hz, 2 |
| | | | H), 7.15 (d, J = 8.1 Hz, 2 H), 6.66 (t, J = 6.1 Hz, 1 H), |
| | | | 4.27 (d, J = 6.0 Hz, 2 H), 4.16 (s, 2 H), 2.07 (tt, J = 8.7, |
| | | | 4.8 Hz, 1 H), 1.03 (dq, J = 6.9, 4.2 Hz, 2 H), 0.85 (p, J = |
| | | | 4.6 Hz, 2 H). |
| 446 | 4-((3- ethyl- 1,2,4- oxadiazol- 5- yl)methyl) aniline | 4-chloro benzyl isocyanate | 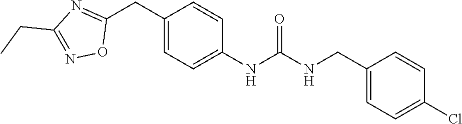 [SMILES: CCc1noc(Cc2ccc(NC(=O)NCc3ccc(Cl)cc3)cc2)n1] |
| | | | 1-(4-chlorobenzyl)-3-(4-((3-ethyl-1,2,4-oxadiazol-5- |
| | | | yl)methyl)phenyl)urea. LCMS-ESI (POS.) m/z: 383.10 |
| | | | (M + H)⁺. ¹H NMR (400 MHz, DMSO-d₆) δ 8.63 (s, 1 H), |
| | | | 7.43-7.35 (m, 4 H), 7.32 (d, J = 8.1 Hz, 2 H), 7.18 (d, J = |
| | | | 8.2 Hz, 2 H), 6.66 (t, J = 6.1 Hz, 1 H), 4.28 (d, J = 5.9 Hz, |
| | | | 2 H), 4.20 (s, 2 H), 2.68 (q, J = 7.5 Hz, 2 H), 1.20 (t, J = |
| | | | 7.5 Hz, 3 H). |
| 447 | 2-(4- amino benzyl)iso thiazolidine- 1,1- dioxide | 4-chloro benzyl isocyanate | 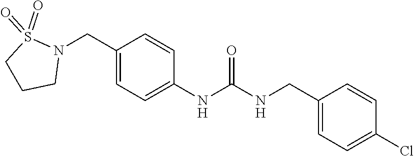 [SMILES: O=C(NCc1ccc(Cl)cc1)Nc1ccc(CN2CCCS2(=O)=O)cc1] |
| | | | 1-(4-chlorobenzyl)-3-(4-((1,1-dioxidoisothiazolidin-2- |
| | | | yl)methyl)phenyl)urea. LCMS-ESI (POS.) m/z: 394.0 |
| | | | (M + H)⁺. ¹H NMR (400 MHz, DMSO-d₆) δ 8.64 (s, 1 H), |
| | | | 7.42-7.36 (m, 4 H), 7.32 (d, J = 8.2 Hz, 2 H), 7.19 (d, J = |
| | | | 8.2 Hz, 2 H), 6.66 (t, J = 6.0 Hz, 1 H), 4.28 (d, J = 5.9 Hz, |
| | | | 2 H), 3.98 (s, 2 H), 3.23 (t, J = 7.7 Hz, 2 H), 3.03 (t, J = |
| | | | 6.8 Hz, 2 H), 2.19 (p, J = 7.1 Hz, 2 H). |
| 448 | 4-((1H- pyrazol- 1- yl)methyl) aniline | 4-chloro benzyl isocyanate | 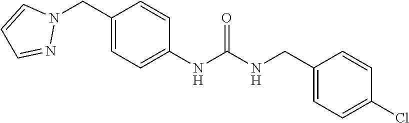 [SMILES: O=C(NCc1ccc(Cl)cc1)Nc1ccc(Cn2cccn2)cc1] |
| | | | 1-(4-((1H-pyrazol-1-yl)methyl)phenyl)-3-(4- |
| | | | chlorobenzyl)urea. LCMS-ESI (POS.) m/z: 341.10 |
| | | | (M + H)⁺. ¹H NMR (400 MHz, DMSO-d₆) δ 8.62 (s, 1 H), |
| | | | 7.76 (s, 1 H), 7.45-7.28 (m, 7 H), 7.12 (d, J = 8.2 Hz, 2 |
| | | | H), 6.64 (t, J = 5.8 Hz, 1 H), 6.24 (s, 1 H), 5.22 (s, 2 H), |
| | | | 4.27 (d, J = 5.9 Hz, 2 H). |
| 452 | 3-(4- amino benzyl) oxazolidin- 2- one | 4-chloro benzyl isocyanate | 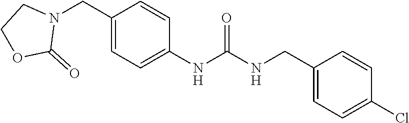 [SMILES: O=C(NCc1ccc(Cl)cc1)Nc1ccc(CN2CCOC2=O)cc1] |
| | | | 1-(4-chlorobenzyl)-3-(4-((2-oxooxazolidin-3- |
| | | | yl)methyl)phenyl)urea. LCMS-ESI (POS.) m/z: 360.00 |
| | | | (M + H)⁺. ¹H NMR (400 MHz, DMSO-d₆) δ 8.64 (s, 1 H), |
| | | | 7.39 (d, J = 8.4 Hz, 4 H), 7.32 (d, J = 8.4 Hz, 2 H), 7.14 |
| | | | (d, J = 8.3 Hz, 2 H), 6.65 (t, J = 6.1 Hz, 1 H), 4.28 (d, J = |
| | | | 5.9 Hz, 2 H), 4.27-4.22 (m, 4 H), 3.38 (dd, J = 8.9, 7.1 |
| | | | Hz, 2 H). |
| 453 | 4-((3,5- dimethyl- 1H- pyrazol- 1- yl)methyl) aniline | 4-chloro benzyl isocyanate | 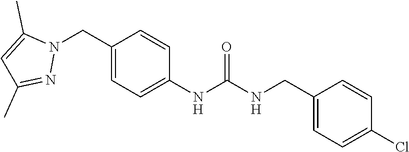 [SMILES: Cc1cc(C)n(Cc2ccc(NC(=O)NCc3ccc(Cl)cc3)cc2)n1] |
| | | | 1-(4-chlorobenzyl)-3-(4-((3,5-dimethyl-1H-pyrazol-1- |
| | | | yl)methyl)phenyl)urea. LCMS-ESI (POS.) m/z: 369.10 |
| | | | (M + H)⁺. ¹H NMR (400 MHz, DMSO-d₆) δ 8.61 (s, 1 H), |
| | | | 7.39 (d, J = 8.3 Hz, 2 H), 7.34 (d, J = 8.4 Hz, 2 H), 7.31 |
| | | | (d, J = 8.4 Hz, 2 H), 6.99 (d, J = 8.2 Hz, 2 H), 6.64 (d, J = |
| | | | 6.0 Hz, 1 H), 5.82 (s, 1 H), 5.07 (s, 2 H), 4.27 (d, J = 5.9 |
| | | | Hz, 2 H), 2.15 (s, 3 H), 2.09 (s, 3 H). |
| 460 | Intermediate 37 | 4-methoxy benzyl isocyanate | 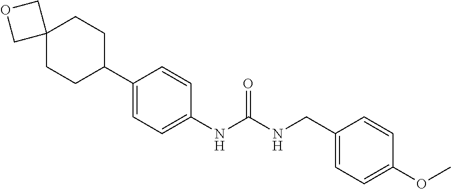 [SMILES: COc1ccc(CNC(=O)Nc2ccc(C3CCC4(CC3)COC4)cc2)cc1] |
| | | | 1-(4-(2-oxaspiro[3.5]nonan-7-yl)phenyl)-3-(4- |
| | | | methoxybenzyl)urea. LCMS-ESI (POS.) m/z: 381.15 |
| | | | (M + H)⁺. ¹H NMR (400 MHz, DMSO-d₆) δ 8.37 (s, 1 H), |
| | | | 7.28 (d, J = 8.5 Hz, 2 H), 7.22 (d, J = 8.6 Hz, 2 H), 7.04 |
| | | | (d, J = 8.5 Hz, 2 H), 6.89 (d, J = 8.6 Hz, 2 H), 6.45 (t, J = |
| | | | 5.9 Hz, 1 H), 4.37 (s, 2 H), 4.23 (s, 2 H), 4.20 (d, J = 5.7 |
| | | | Hz, 2 H), 3.73 (s, 3 H), 2.35 (tt, J = 11.7, 3.4 Hz, 1 H), |
| | | | 2.13 (d, J = 12.6 Hz, 2 H), 1.73-1.63 (m, 2 H), 1.50 (td, J = |
| | | | 13.0, 3.5 Hz, 2 H), 1.32 (qd, J = 13.1, 3.3 Hz, 2 H). |
| 474 | Intermediate 13.34 | 4-chloro benzyl isocyanate | 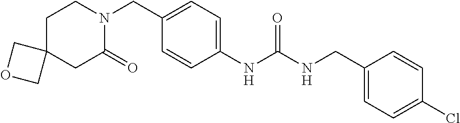 [SMILES: O=C(NCc1ccc(Cl)cc1)Nc1ccc(CN2CCC3(COC3)CC2=O)cc1] |
| | | | 1-(4-chlorobenzyl)-3-(4-((6-oxo-2-oxa-7- |
| | | | azaspiro[3.5]nonan-7-yl)methyl)phenyl)urea. LCMS- |
| | | | ESI (POS.) m/z: 414.1 (M + H)⁺. ¹H NMR (400 MHz, |
| | | | DMSO-d₆) δ 8.63 (s, 1 H), 7.39 (d, J = 8.5 Hz, 2 H), 7.34 |
| | | | (d, J = 8.5 Hz, 2 H), 7.31 (d, J = 8.4 Hz, 2 H), 7.04 (d, J = |
| | | | 8.3 Hz, 2 H), 6.68 (t, J = 6.0 Hz, 1 H), 4.38 (s, 2 H), 4.35 |
| | | | (d, J = 5.8 Hz, 2 H), 4.31 (d, J = 5.8 Hz, 2 H), 4.27 (d, J = |
| | | | 6.0 Hz, 2 H), 3.13 (t, J = 6.2 Hz, 2 H), 2.63 (s, 2 H), 2.00 |
| | | | (t, J = 6.2 Hz, 2 H). |
| 484 | Intermediate 13.35 | 4-methoxy benzyl isocyanate | 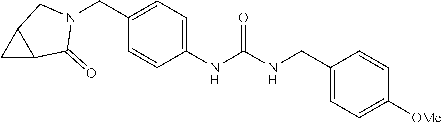 [SMILES: COc1ccc(CNC(=O)Nc2ccc(CN3CC4CC4C3=O)cc2)cc1] |
| | | | 1-(4-methoxybenzyl)-3-(4-((2-oxo-3- |
| | | | azabicyclo[3.1.0]hexan-3-yl)methyl)phenyl)urea. |
| | | | LCMS-ESI (POS.) m/z: 366.1 (M + H)⁺. ¹H NMR (400 |
| | | | MHz, DMSO-d₆) δ 8.50 (s, 1 H), 7.35 (d, J = 8.5 Hz, 2 H), |
| | | | 7.22 (d, J = 8.6 Hz, 2 H), 7.01 (d, J = 8.5 Hz, 2 H), 6.89 |
| | | | (d, J = 8.6 Hz, 2 H), 6.50 (t, J = 5.9 Hz, 1 H), 4.21 (d, J = |
| | | | 5.8 Hz, 2 H), 4.14 (s, 2 H), 3.73 (s, 3 H), 3.30 (d, J = 5.4 |
| | | | Hz, 1 H), 3.08 (dd, J = 10.5, 1.7 Hz, 1 H), 1.89-1.76 (m, 2 |
| | | | H), 1.04 (td, J = 8.0, 4.2 Hz, 1 H), 0.42 (q, J = 3.9 Hz, 1 |
| | | | H). |
| 483 | Intermediate 13.35 | 4-chloro benzyl isocyanate | 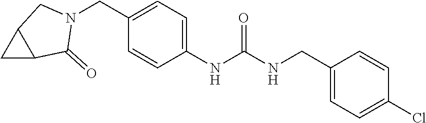 [SMILES: O=C(NCc1ccc(Cl)cc1)Nc1ccc(CN2CC3CC3C2=O)cc1] |
| | | | 1-(4-chlorobenzyl)-3-(4-((2-oxo-3- |
| | | | azabicyclo[3.1.0]hexan-3-yl)methyl)phenyl)urea. |
| | | | LCMS-ESI (POS.) m/z: 370.10 (M + H)⁺. ¹H NMR (400 |
| | | | MHz, DMSO-d₆) δ 8.65 (s, 1 H), 7.43 (d, J = 8.4 Hz, 2 H), |
| | | | 7.40 (d, J = 8.5 Hz, 2 H), 7.36 (d, J = 8.5 Hz, 2 H), 7.06 |
| | | | (d, J = 8.5 Hz, 2 H), 6.68 (t, J = 6.1 Hz, 1 H), 4.32 (d, J = |
| | | | 6.0 Hz, 2 H), 4.18 (s, 2 H), 3.36 (dd, J = 10.4, 5.4 Hz, 1 |
| | | | H), 3.12 (d, J = 10.6 Hz, 1 H), 1.93-1.81 (m, 2 H), 1.08 |
| | | | (td, J = 8.0, 4.2 Hz, 1 H), 0.47 (q, J = 3.9 Hz, 1 H). |
| 489 | 4-(4- amino phenoxy)- N- methyl pico linamide | 4-methoxy benzyl isocyanate | 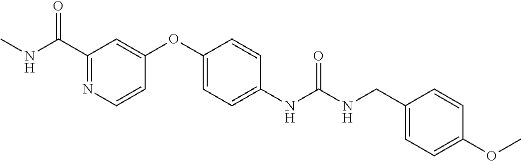 |
| | | | 4-(4-(3-(4-methoxybenzyl)ureido)phenoxy)-N- |
| | | | methylpicolinamide. LCMS-ESI (POS.) m/z: 407.2 |
| | | | (M + H)⁺. ¹H NMR (400 MHz, DMSO-d₆) δ 8.77 (q, J = 4.6 |
| | | | Hz, 1 H), 8.68 (s, 1 H), 8.49 (d, J = 5.6 Hz, 1 H), 7.52 (d, J = |
| | | | 8.9 Hz, 2 H), 7.36 (d, J = 2.6 Hz, 1 H), 7.24 (d, J = 8.6 |
| | | | Hz, 2 H), 7.14-7.07 (m, 3 H), 6.90 (d, J = 8.6 Hz, 2 H), |
| | | | 6.58 (t, J = 5.9 Hz, 1 H), 4.24 (d, J = 5.8 Hz, 2 H), 3.74 (s, |
| | | | 3 H), 2.78 (d, J = 4.8 Hz, 3 H). |
| 490 | 4- (pyridin- 3- ylmethoxy) aniline | 4-chloro benzyl isocyanate | 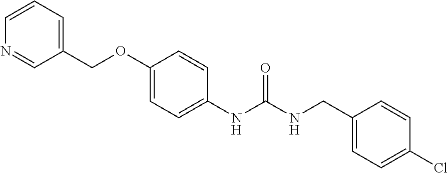 [SMILES: O=C(NCc1ccc(Cl)cc1)Nc1ccc(OCc2cccnc2)cc1] |
| | | | 1-(4-chlorobenzyl)-3-(4-(pyridin-3- |
| | | | ylmethoxy)phenyl)urea. LCMS-ESI (POS.) m/z: 368.10 |
| | | | (M + H)⁺. ¹H NMR (400 MHz, DMSO-d₆) δ 8.65 (d, J = 2.1 |
| | | | Hz, 1 H), 8.54 (dd, J = 4.8, 1.7 Hz, 1 H), 8.43 (s, 1 H), |
| | | | 7.85 (dt, J = 7.8, 2.0 Hz, 1 H), 7.44-7.36 (m, 3 H), 7.34- |
| | | | 7.28 (m, 4 H), 6.92 (d, J = 9.0 Hz, 2 H), 6.57 (t, J = 6.0 |
| | | | Hz, 1 H), 5.09 (s, 2 H), 4.27 (d, J = 5.9 Hz, 2 H). |
| 488 | 4- (pyridin- 4- ylmethoxy) aniline | 4-methoxy benzyl isocyanate | 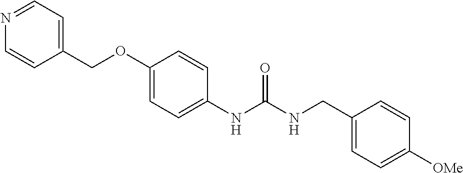 [SMILES: COc1ccc(CNC(=O)Nc2ccc(OCc3ccncc3)cc2)cc1] |
| | | | 1-(4-methoxybenzyl)-3-(4-(pyridin-4- |
| | | | ylmethoxy)phenyl)urea. LCMS-ESI (POS.) m/z: 368.10 |
| | | | (M + H)⁺. ¹H NMR (400 MHz, DMSO-d₆) δ 8.57 (d, J = 6.0 |
| | | | Hz, 2 H), 8.34 (s, 1 H), 7.42 (d, J = 5.9 Hz, 2 H), 7.30 (d, J = |
| | | | 8.9 Hz, 2 H), 7.22 (d, J = 8.6 Hz, 2 H), 6.90 (d, J = 5.6 |
| | | | Hz, 2 H), 6.88 (d, J = 5.1 Hz, 2 H), 6.44 (t, J = 5.9 Hz, 1 |
| | | | H), 5.12 (s, 2 H), 4.20 (d, J = 5.8 Hz, 2 H), 3.73 (s, 3 H). |
| 495 | 4-(2- methyl oxazol- 5- yl)aniline | 4-methoxy benzyl isocyanate | 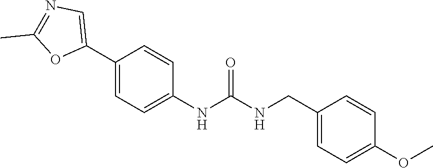 |
| | | | 1-(4-methoxybenzyl)-3-(4-(2-methyloxazol-5- |
| | | | yl)phenyl)urea. LCMS-ESI (POS.) m/z: 338.10 (M + H)⁺. |
| | | | ¹H NMR (400 MHz, DMSO-d₆) δ 8.69 (s, 1 H), 7.52 (d, J = |
| | | | 8.8 Hz, 2 H), 7.48 (d, J = 8.8 Hz, 2 H), 7.34 (s, 1 H), |
| | | | 7.23 (d, J = 8.6 Hz, 2 H), 6.90 (d, J = 8.6 Hz, 2 H), 6.59 (t, |
| | | | J = 5.9 Hz, 1 H), 4.23 (d, J = 5.8 Hz, 2 H), 3.73 (s, 3 H), |
| | | | 2.45 (s, 3 H). |
| 496 | 4- ((isopropyl sulfonyl) methyl) aniline | 4-chloro benzyl isocyanate | 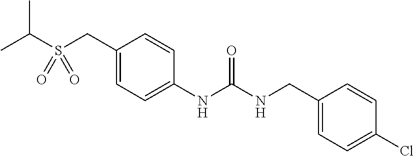 [SMILES: CC(C)S(=O)(=O)Cc1ccc(NC(=O)NCc2ccc(Cl)cc2)cc1] |
| | | | 1-(4-chlorobenzyl)-3-(4- |
| | | | ((isopropylsulfonyl)methyl)phenyl)urea. LCMS-ESI |
| | | | (POS.) m/z: 381.10 (M + H)⁺. ¹H NMR (400 MHz, DMSO- |
| | | | d₆) δ 8.70 (s, 1 H), 7.44-7.36 (m, 4 H), 7.32 (d, J = 8.5 Hz, |
| | | | 2 H), 7.25 (d, J = 8.6 Hz, 2 H), 6.69 (t, J = 6.0 Hz, 1 H), |
| | | | 4.34 (s, 2 H), 4.29 (d, J = 5.9 Hz, 2 H), 3.12 (hept, J = 6.6 |
| | | | Hz, 1 H), 1.25 (d, J = 6.8 Hz, 6 H). |
| 497 | 4- ((isopropyl sulfonyl) methyl) aniline | 4-methoxy benzyl isocyanate | 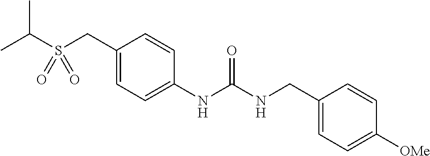 [SMILES: COc1ccc(CNC(=O)Nc2ccc(CS(=O)(=O)C(C)C)cc2)cc1] |
| | | | 1-(4-methoxybenzyl)-3-(4- |
| | | | ((isopropylsulfonyl)methyl)phenyl)urea. LCMS-ESI |
| | | | (POS.) m/z: 377.10 (M + H)⁺. ¹H NMR (400 MHz, DMSO- |
| | | | d₆) δ 8.60 (s, 1 H), 7.41 (d, J = 8.6 Hz, 2 H), 7.25 (d, J = |
| | | | 6.1 Hz, 2 H), 7.22 (d, J = 6.1 Hz, 2 H), 6.89 (d, J = 8.6 Hz, |
| | | | 2 H), 6.56 (t, J = 5.8 Hz, 1 H), 4.34 (s, 2 H), 4.22 (d, J = |
| | | | 5.8 Hz, 2 H), 3.73 (s, 3 H), 3.12 (hept, J = 7.1 Hz, 1 H), |
| | | | 1.25 (d, J = 6.8 Hz, 6 H). |
| 498 | 4- amino- N-(1- phenyl- 1H- pyrazol- 5- yl)benzene sulfonamide | 4-chloro benzyl isocyanate | 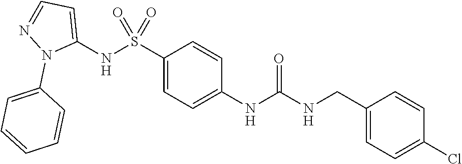 [SMILES: O=C(NCc1ccc(Cl)cc1)Nc1ccc(S(=O)(=O)Nc2ccnn2-c2ccccc2)cc1] |
| | | | 4-(3-(4-chlorobenzyl)ureido)-N-(1-phenyl-1H-pyrazol- |
| | | | 5-yl)benzenesulfonamide. LCMS-ESI (POS.) m/z: |
| | | | 482.10 (M + H)⁺. ¹H NMR (400 MHz, DMSO-d₆) δ 8.95 (s, |
| | | | 1 H), 7.73 (s, 2 H), 7.57-7.50 (m, 3 H), 7.50-7.37 (m, 6 |
| | | | H), 7.36-7.22 (m, 4 H), 6.78 (s, 1 H), 5.63 (s, 1 H), 4.29 |
| | | | (d, J = 6.0 Hz, 2 H). |
| 499 | 4- (pyridin- 4- ylmethyl) aniline | 4-methoxy benzyl isocyanate | 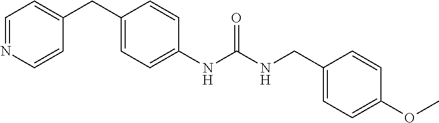 [SMILES: COc1ccc(CNC(=O)Nc2ccc(Cc3ccncc3)cc2)cc1] |
| | | | 1-(4-methoxybenzyl)-3-(4-(pyridin-4- |
| | | | ylmethyl)phenyl)urea. LCMS-ESI (POS.) m/z: 348.10 |
| | | | (M + H)⁺. ¹H NMR (400 MHz, DMSO-d₆) δ 8.48-8.39 (m, |
| | | | 3 H), 7.33 (d, J = 8.4 Hz, 2 H), 7.25-7.17 (m, 4 H), 7.10 |
| | | | (d, J = 8.2 Hz, 2 H), 6.89 (d, J = 8.6 Hz, 2 H), 6.47 (t, J = |
| | | | 5.9 Hz, 1 H), 4.20 (d, J = 5.8 Hz, 2 H), 3.87 (s, 2 H), 3.73 |
| | | | (s, 3 H). |
| 502 | 4- (((tetra hydro furan-2- yl)methyl) sulfonyl) aniline | 4-chloro benzyl isocyanate | 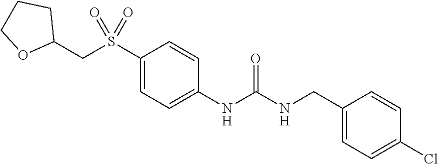 [SMILES: O=C(NCc1ccc(Cl)cc1)Nc1ccc(S(=O)(=O)CC2CCCO2)cc1] |
| | | | 1-(4-chlorobenzyl)-3-(4-(((tetrahydrofuran-2- |
| | | | yl)methyl)sulfonyl)phenyl)urea. LCMS-ESI (POS.) m/z: |
| | | | 409.00 (M + H)⁺. ¹H NMR (400 MHz, DMSO-d₆) δ 9.27 (s, |
| | | | 1 H), 7.73 (d, J = 8.9 Hz, 2 H), 7.63 (d, J = 8.9 Hz, 2 H), |
| | | | 7.40 (d, J = 8.5 Hz, 2 H), 7.33 (d, J = 8.5 Hz, 2 H), 6.99 (t, |
| | | | J = 6.0 Hz, 1 H), 4.30 (d, J = 5.9 Hz, 2 H), 4.03 (p, J = 6.4 |
| | | | Hz, 1 H), 3.62 (td, J = 7.7, 6.4 Hz, 1 H), 3.53 (td, J = 7.9, |
| | | | 6.1 Hz, 1 H), 3.43 (dd, J = 6.1, 4.4 Hz, 2 H), 1.93 (dddd, J = |
| | | | 12.1, 8.5, 6.7, 5.4 Hz, 1 H), 1.85-1.67 (m, 2 H), 1.54 |
| | | | (ddt, J = 12.1, 8.5, 6.8 Hz, 1 H). |
| 503 | 4- (pyridin- 3- yl)aniline | 4-chloro benzyl isocyanate | 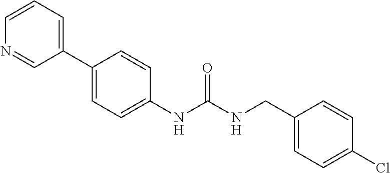 [SMILES: O=C(NCc1ccc(Cl)cc1)Nc1ccc(-c2cccnc2)cc1] |
| | | | 1-(4-chlorobenzyl)-3-(4-(pyridin-3-yl)phenyl)urea. |
| | | | LCMS-ESI (POS.) m/z: 338.10 (M + H)⁺. ¹H NMR (400 |
| | | | MHz, DMSO-d₆) δ 8.86 (d, J = 2.3 Hz, 1 H), 8.78 (s, 1 H), |
| | | | 8.51 (dd, J = 4.7, 1.6 Hz, 1 H), 8.02 (dt, J = 8.0, 2.0 Hz, 1 |
| | | | H), 7.63 (d, J = 8.7 Hz, 2 H), 7.54 (d, J = 8.7 Hz, 2 H), |
| | | | 7.44 (dd, J = 7.9, 4.7 Hz, 1 H), 7.40 (d, J = 8.5 Hz, 2 H), |
| | | | 7.34 (d, J = 8.5 Hz, 2 H), 6.73 (t, J = 6.0 Hz, 1 H), 4.31 (d, |
| | | | J = 5.9 Hz, 2 H). |
| 504 | 4- (pyridin- 3- yl)aniline | 4-methoxy benzyl isocyanate | 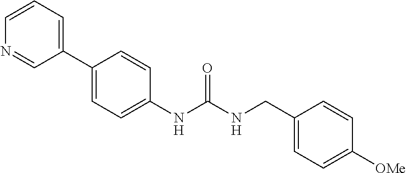 [SMILES: COc1ccc(CNC(=O)Nc2ccc(-c3cccnc3)cc2)cc1] |
| | | | 1-(4-methoxybenzyl)-3-(4-(pyridin-3-yl)phenyl)urea. |
| | | | LCMS-ESI (POS.) m/z: 334.10 (M + H)⁺. ¹H NMR (400 |
| | | | MHz, DMSO-d₆) δ 8.86 (d, J = 2.3 Hz, 1 H), 8.68 (s, 1 H), |
| | | | 8.50 (dd, J = 4.8, 1.6 Hz, 1 H), 8.02 (dt, J = 7.9, 1.9 Hz, 1 |
| | | | H), 7.62 (d, J = 8.7 Hz, 2 H), 7.54 (d, J = 8.7 Hz, 2 H), |
| | | | 7.44 (dd, J = 8.0, 4.7 Hz, 1 H), 7.25 (d, J = 8.6 Hz, 2 H), |
| | | | 6.91 (d, J = 8.6 Hz, 2 H), 6.59 (t, J = 5.9 Hz, 1 H), 4.24 (d, |
| | | | J = 5.8 Hz, 2 H), 3.74 (s, 3 H). |
| 505 | 4- (((tetra hydro furan-3- yl)methyl) sulfonyl) aniline | 4-chloro benzyl isocyanate | 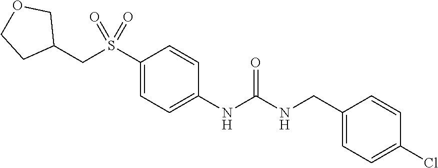 [SMILES: O=C(NCc1ccc(Cl)cc1)Nc1ccc(S(=O)(=O)CC2CCOC2)cc1] |
| | | | 1-(4-chlorobenzyl)-3-(4-(((tetrahydrofuran-3- |
| | | | yl)methyl)sulfonyl)phenyl)urea. LCMS-ESI (POS.) m/z: |
| | | | 409.1 (M + H)⁺. ¹H NMR (400 MHz, DMSO-d₆) δ 9.22 (s, 1 |
| | | | H), 7.75 (d, J = 8.9 Hz, 2 H), 7.65 (d, J = 8.9 Hz, 2 H), |
| | | | 7.40 (d, J = 8.5 Hz, 2 H), 7.33 (d, J = 8.5 Hz, 2 H), 6.90 (t, |
| | | | J = 6.0 Hz, 1 H), 4.31 (d, J = 5.9 Hz, 2 H), 4.10 (q, J = 5.3 |
| | | | Hz, 1 H), 3.75 (dd, J = 8.5, 7.3 Hz, 1 H), 3.67 (td, J = 8.2, |
| | | | 4.9 Hz, 1 H), 3.57 (q, J = 7.5 Hz, 1 H), 3.43-3.27 (m, 2 |
| | | | H), 2.37 (dt, J = 14.8, 7.4 Hz, 1 H), 1.96 (dtd, J = 12.5, |
| | | | 7.6, 4.9 Hz, 1 H), 1.56 (dq, J = 12.3, 7.8 Hz, 1 H). |
| 506 | 2-(4- amino phenoxy)- 1- morpholino ethan-1- one | 4-chloro benzyl isocyanate | 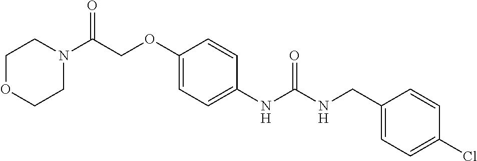 [SMILES: O=C(NCc1ccc(Cl)cc1)Nc1ccc(OCC(=O)N2CCOCC2)cc1] |
| | | | 1-(4-chlorobenzyl)-3-(4-(2-morpholino-2- |
| | | | oxoethoxy)phenyl)urea. LCMS-ESI (POS.) m/z: 404.10 |
| | | | (M + H)⁺. ¹H NMR (400 MHz, DMSO-d₆) δ 8.40 (s, 1 H), |
| | | | 7.39 (d, J = 8.5 Hz, 2 H), 7.31 (d, J = 8.4 Hz, 2 H), 7.28 |
| | | | (d, J = 9.0 Hz, 2 H), 6.82 (d, J = 9.0 Hz, 2 H), 6.55 (t, J = |
| | | | 6.1 Hz, 1 H), 4.73 (s, 2 H), 4.27 (d, J = 5.9 Hz, 2 H), 3.58 |
| | | | (dt, J = 14.7, 4.6 Hz, 4 H), 3.45 (p, J = 4.7 Hz, 4 H). |
| 507 | 4- (oxetan- 3- yl)aniline | 4-chloro benzyl isocyanate | 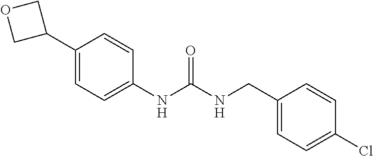 [SMILES: O=C(NCc1ccc(Cl)cc1)Nc1ccc(C2COC2)cc1] |
| | | | 1-(4-chlorobenzyl)-3-(4-(oxetan-3-yl)phenyl)urea. |
| | | | LCMS-ESI (POS.) m/z: 317.10 (M + H)⁺. ¹H NMR (400 |
| | | | MHz, DMSO-d₆) δ 8.59 (s, 1 H), 7.39 (d, J = 8.3 Hz, 4 H), |
| | | | 7.32 (d, J = 8.2 Hz, 2 H), 7.26 (d, J = 8.3 Hz, 2 H), 6.63 (t, |
| | | | J = 6.0 Hz, 1 H), 4.90 (dd, J = 8.4, 5.8 Hz, 2 H), 4.57 (t, J = |
| | | | 6.3 Hz, 2 H), 4.28 (d, J = 5.9 Hz, 2 H), 4.16 (p, J = 7.7 |
| | | | Hz, 1 H). |
| 508 | 4-(1- methyl- 1H- pyrazol- 3- yl)aniline | 4-chloro benzyl isocyanate | 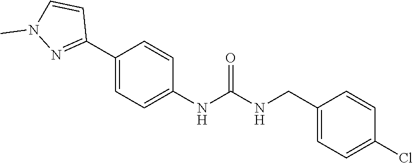 [SMILES: Cn1ccc(-c2ccc(NC(=O)NCc3ccc(Cl)cc3)cc2)n1] |
| | | | 1-(4-chlorobenzyl)-3-(4-(1-methyl-1H-pyrazol-3- |
| | | | yl)phenyl)urea. LCMS-ESI (POS.) m/z: 341.05 (M + H)⁺. |
| | | | ¹H NMR (400 MHz, DMSO-d₆) δ 8.59 (s, 1 H), 8.00 (s, 1 |
| | | | H), 7.75 (s, 1 H), 7.45-7.35 (m, 6 H), 7.33 (d, J = 8.3 Hz, |
| | | | 2 H), 6.64 (t, J = 6.0 Hz, 1 H), 4.29 (d, J = 5.9 Hz, 2 H), |
| | | | 3.84 (s, 3 H). |
| 514 | 4- (morpholino sulfonyl) aniline | 4-chloro benzyl isocyanate | 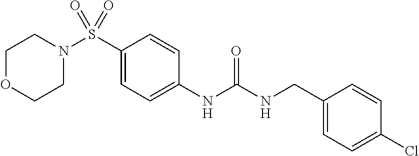 [SMILES: O=C(NCc1ccc(Cl)cc1)Nc1ccc(S(=O)(=O)N2CCOCC2)cc1] |
| | | | 1-(4-chlorobenzyl)-3-(4- |
| | | | (morpholinosulfonyl)phenyl)urea. LCMS-ESI (POS.) |
| | | | m/z: 410.1 (M + H)⁺. ¹H NMR (400 MHz, DMSO-d₆) δ |
| | | | 9.23 (s, 1 H), 7.66 (d, J = 8.9 Hz, 2 H), 7.59 (d, J = 8.9 Hz, |
| | | | 2 H), 7.40 (d, J = 8.5 Hz, 2 H), 7.33 (d, J = 8.5 Hz, 2 H), |
| | | | 6.91 (t, J = 6.0 Hz, 1 H), 4.31 (d, J = 5.9 Hz, 2 H), 3.62 (t, |
| | | | J = 4.7 Hz, 4 H), 2.81 (dd, J = 5.7, 3.6 Hz, 4 H). |
| 515 | 4- ((tetrahydro- 2H- pyran- 4- yl)methoxy) aniline | 4-chloro benzyl isocyanate | 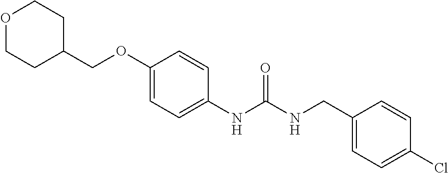 [SMILES: O=C(NCc1ccc(Cl)cc1)Nc1ccc(OCC2CCOCC2)cc1] |
| | | | 1-(4-chlorobenzyl)-3-(4-((tetrahydro-2H-pyran-4- |
| | | | yl)methoxy)phenyl)urea. LCMS-ESI (POS.) m/z: 375.10 |
| | | | (M + H)⁺. ¹H NMR (400 MHz, DMSO-d₆) δ 8.38 (s, 1 H), |
| | | | 7.39 (d, J = 8.4 Hz, 2 H), 7.31 (d, J = 8.5 Hz, 2 H), 7.28 |
| | | | (d, J = 9.0 Hz, 2 H), 6.81 (d, J = 9.0 Hz, 2 H), 6.54 (t, J = |
| | | | 6.1 Hz, 1 H), 4.26 (d, J = 5.9 Hz, 2 H), 3.87 (ddd, J = 11.5, |
| | | | 4.5, 1.8 Hz, 2 H), 3.76 (d, J = 6.5 Hz, 2 H), 3.32 (td, J = |
| | | | 11.7, 2.1 Hz, 2 H), 1.95 (dtd, J = 14.1, 7.6, 6.7, 3.3 Hz, 1 |
| | | | H), 1.66 (ddd, J = 12.8, 4.0, 1.9 Hz, 2 H), 1.30 (qd, J = |
| | | | 12.2, 4.5 Hz, 2 H). |
| 516 | 4- ((cyclo pentyloxy) methyl) aniline | 4-chloro benzyl isocyanate | 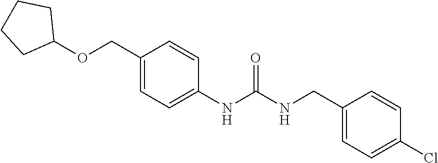 [SMILES: O=C(NCc1ccc(Cl)cc1)Nc1ccc(COC2CCCC2)cc1] |
| | | | 1-(4-chlorobenzyl)-3-(4- |
| | | | ((cyclopentyloxy)methyl)phenyl)urea. LCMS-ESI |
| | | | (POS.) m/z: 359.10 (M + H)⁺. ¹H NMR (400 MHz, DMSO- |
| | | | d₆) δ 8.58 (s, 1 H), 7.39 (d, J = 8.5 Hz, 2 H), 7.36 (d, J = |
| | | | 8.5 Hz, 2 H), 7.32 (d, J = 8.5 Hz, 2 H), 7.16 (d, J = 8.5 Hz, |
| | | | 2 H), 6.63 (t, J = 6.0 Hz, 1 H), 4.31 (s, 2 H), 4.28 (d, J = |
| | | | 5.9 Hz, 2 H), 3.93 (tt, J = 6.1, 3.3 Hz, 1 H), 1.76-1.55 (m, |
| | | | 6 H), 1.55-1.39 (m, 2 H). |
| 517 | 4-(2- (pyridin- 2- yl)ethoxy) aniline | 4-chloro benzyl isocyanate | 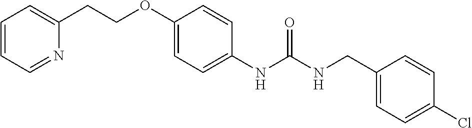 [SMILES: O=C(NCc1ccc(Cl)cc1)Nc1ccc(OCCc2ccccn2)cc1] |
| | | | 1-(4-chlorobenzyl)-3-(4-(2-(pyridin-2- |
| | | | yl)ethoxy)phenyl)urea. LCMS-ESI (POS.) m/z: 382.10 |
| | | | (M + H)⁺. ¹H NMR (400 MHz, DMSO-d₆) δ 8.51 (dt, J = |
| | | | 4.8, 1.4 Hz, 1 H), 8.37 (s, 1 H), 7.73 (td, J = 7.6, 1.9 Hz, 1 |
| | | | H), 7.39 (d, J = 8.4 Hz, 2 H), 7.36 (d, J = 7.7 Hz, 1 H), |
| | | | 7.31 (d, J = 8.5 Hz, 2 H), 7.27 (d, J = 9.0 Hz, 2 H), 7.26- |
| | | | 7.21 (m, 1 H), 6.81 (d, J = 9.0 Hz, 2 H), 6.54 (t, J = 6.1 |
| | | | Hz, 1 H), 4.28 (t, J = 5.7 Hz, 2 H), 4.26 (d, J = 4.1 Hz, 2 |
| | | | H), 3.15 (t, J = 6.6 Hz, 2 H). |
| 518 | 4-(2- (pyridin- 2- yl)ethoxy) aniline | 4-methoxy benzyl isocyanate | 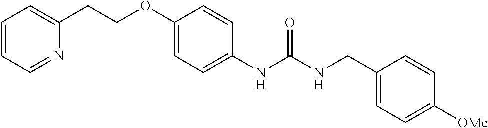 [SMILES: COc1ccc(CNC(=O)Nc2ccc(OCCc3ccccn3)cc2)cc1] |
| | | | 1-(4-chlorobenzyl)-3-(4-(2-(pyridin-2- |
| | | | yl)ethoxy)phenyl)urea. LCMS-ESI (POS.) m/z: 378.10 |
| | | | (M + H)⁺. ¹H NMR (400 MHz, DMSO-d₆) δ 8.51 (dt, J = |
| | | | 4.7, 1.5 Hz, 1 H), 8.29 (s, 1 H), 7.73 (td, J = 7.7, 1.9 Hz, 1 |
| | | | H), 7.36 (d, J = 7.8 Hz, 1 H), 7.27 (d, J = 9.0 Hz, 2 H), |
| | | | 7.24 (dd, J = 2.6, 1.0 Hz, 1 H), 7.22 (d, J = 8.5 Hz, 2 H), |
| | | | 6.89 (d, J = 8.6 Hz, 2 H), 6.80 (d, J = 9.0 Hz, 2 H), 6.41 (t, |
| | | | J = 5.9 Hz, 1 H), 4.28 (t, J = 6.7 Hz, 2 H), 4.20 (d, J = 5.8 |
| | | | Hz, 2 H), 3.73 (s, 3 H), 3.15 (t, J = 6.6 Hz, 2 H). |
| 519 | 4- amino- N- methyl benzene sulfonamide | 4-chloro benzyl isocyanate | 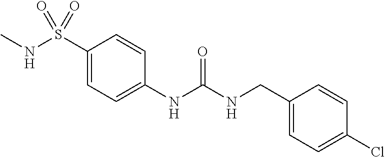 [SMILES: CNS(=O)(=O)c1ccc(NC(=O)NCc2ccc(Cl)cc2)cc1] |
| | | | 4-(3-(4-chlorobenzyl)ureido)-N- |
| | | | methylbenzenesulfonamide. LCMS-ESI (POS.) m/z: |
| | | | 354.00 (M + H)⁺. ¹H NMR (400 MHz, DMSO-d₆) δ 9.09 (s, |
| | | | 1 H), 7.63 (d, J = 9.1 Hz, 2 H), 7.59 (d, J = 9.1 Hz, 2 H), |
| | | | 7.40 (d, J = 8.4 Hz, 2 H), 7.33 (d, J = 8.5 Hz, 2 H), 7.22 |
| | | | (q, J = 5.1 Hz, 1 H), 6.85 (t, J = 6.0 Hz, 1 H), 4.30 (d, J = |
| | | | 5.9 Hz, 2 H), 2.37 (d, J = 5.0 Hz, 3 H). |
| 520 | N-(4- amino phenyl) cyclo propane carboxamide | 4-chloro benzyl isocyanate | 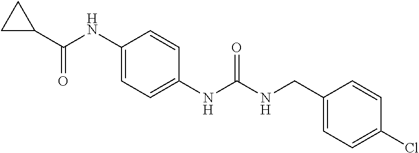 [SMILES: O=C(NCc1ccc(Cl)cc1)Nc1ccc(NC(=O)C2CC2)cc1] |
| | | | N-(4-(3-(4- |
| | | | chlorobenzyl)ureido)phenyl)cyclopropanecarboxamide. |
| | | | LCMS-ESI (POS.) m/z: 344.10 (M + H)⁺. ¹H NMR (400 |
| | | | MHz, DMSO-d₆) δ 10.01 (s, 1 H), 8.48 (s, 1 H), 7.43 (d, J = |
| | | | 8.9 Hz, 2 H), 7.39 (d, J = 8.5 Hz, 2 H), 7.34-7.27 (m, 4 |
| | | | H), 6.59 (t, J = 6.1 Hz, 1 H), 4.27 (d, J = 6.0 Hz, 2 H), |
| | | | 1.77-1.68 (m, 1 H), 0.80-0.71 (m, 4 H). |
| 521 | 4- (pyridin- 2- ylmethoxy) aniline | 4-chloro benzyl isocyanate | 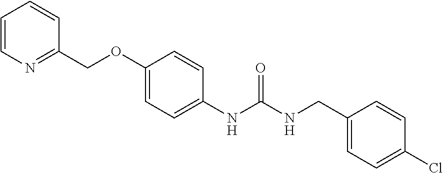 [SMILES: O=C(NCc1ccc(Cl)cc1)Nc1ccc(OCc2ccccn2)cc1] |
| | | | 1-(4-chlorobenzyl)-3-(4-(pyridin-2- |
| | | | ylmethoxy)phenyl)urea. LCMS-ESI (POS.) m/z: 368.10 |
| | | | (M + H)⁺. ¹H NMR (400 MHz, DMSO-d₆) δ 8.57 (dt, J = |
| | | | 4.8, 1.3 Hz, 1 H), 8.41 (s, 1 H), 7.83 (td, J = 7.7, 1.8 Hz, 1 |
| | | | H), 7.50 (d, J = 7.8 Hz, 1 H), 7.39 (d, J = 8.4 Hz, 2 H), |
| | | | 7.36-7.27 (m, 5 H), 6.91 (d, J = 9.0 Hz, 2 H), 6.56 (t, J = |
| | | | 6.1 Hz, 1 H), 5.11 (s, 2 H), 4.27 (d, J = 6.0 Hz, 2 H). |
| 522 | N-(4- amino phenyl)- 2- morpholino acetamide | 4-chloro benzyl isocyanate | 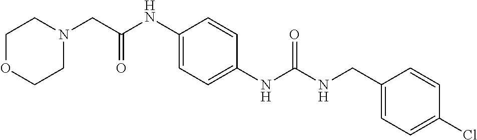 [SMILES: O=C(CN1CCOCC1)Nc1ccc(NC(=O)NCc2ccc(Cl)cc2)cc1] |
| | | | N-(4-(3-(4-chlorobenzyl)ureido)phenyl)-2- |
| | | | morpholinoacetamide. LCMS-ESI (POS.) m/z: 403.1 |
| | | | (M + H)⁺. ¹H NMR (400 MHz, DMSO-d₆) δ 9.57 (s, 1 H), |
| | | | 8.53 (s, 1 H), 7.47 (d, J = 8.6 Hz, 2 H), 7.39 (d, J = 8.2 Hz, |
| | | | 2 H), 7.36-7.28 (m, 4 H), 6.61 (t, J = 6.0 Hz, 1 H), 4.27 |
| | | | (d, J = 5.9 Hz, 2 H), 3.72-3.56 (m, 4 H), 3.09 (s, 2 H), |
| | | | 2.58-2.42 (m, 4 H). |
| 523 | N-(4- amino phenyl)- 2- morpholino acetamide | 4-methoxy benzyl isocyanate | 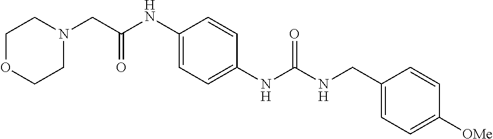 [SMILES: COc1ccc(CNC(=O)Nc2ccc(NC(=O)CN3CCOCC3)cc2)cc1] |
| | | | N-(4-(3-(4-methoxybenzyl)ureido)phenyl)-2- |
| | | | morpholinoacetamide. LCMS-ESI (POS.) m/z: 399.2 |
| | | | (M + H)⁺. ¹H NMR (400 MHz, DMSO-d₆) δ 9.57 (s, 1 H), |
| | | | 8.43 (s, 1 H), 7.47 (d, J = 8.9 Hz, 2 H), 7.32 (d, J = 8.9 Hz, |
| | | | 2 H), 7.22 (d, J = 8.6 Hz, 2 H), 6.89 (d, J = 8.6 Hz, 2 H), |
| | | | 6.47 (t, J = 5.9 Hz, 1 H), 4.21 (d, J = 5.8 Hz, 2 H), 3.73 (s, |
| | | | 3 H), 3.64 (t, J = 4.5 Hz, 4 H), 3.10 (s, 2 H), 2.58-2.43 (m, |
| | | | 4 H). |
| 524 | Intermediate 13.36 | 4-chloro benzyl isocyanate | 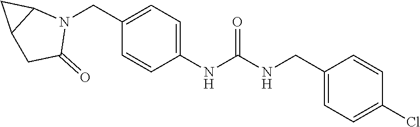 [SMILES: O=C(NCc1ccc(Cl)cc1)Nc1ccc(CN2C(=O)CC3CC32)cc1] |
| | | | 1-(4-chlorophenyl)-3-(4-((3-oxo-2- |
| | | | azabicyclo[3.1.0]hexan-2-yl)methyl)phenyl)urea. |
| | | | LCMS-ESI (POS.) m/z: 370.10 (M + H)⁺. ¹H NMR (400 |
| | | | MHz, DMSO-d₆) δ 8.55 (s, 1 H), 7.37-7.28 (m, 4 H), 7.26 |
| | | | (d, J = 8.5 Hz, 2 H), 7.07 (d, J = 8.5 Hz, 2 H), 6.58 (t, J = |
| | | | 6.0 Hz, 1 H), 4.30-4.16 (m, 4 H), 2.89 (ddt, J = 7.1, 5.3, |
| | | | 1.7 Hz, 1 H), 2.63 (dd, J = 17.8, 7.3 Hz, 1 H), 2.17 (d, J = |
| | | | 17.8 Hz, 1 H), 1.36 (qd, J = 7.5, 4.6 Hz, 1 H), 0.66 (dt, J = |
| | | | 8.3, 5.2 Hz, 1 H), 0.00 (td, J = 5.1, 2.2 Hz, 1 H). |
| 525 | Intermediate 13.36 | 4-methoxy benzyl isocyanate | 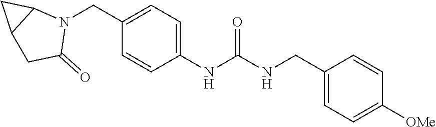 [SMILES: COc1ccc(CNC(=O)Nc2ccc(CN3C(=O)CC4CC43)cc2)cc1] |
| | | | 1-(4-methoxybenzyl)-3-(4-((3-oxo-2- |
| | | | azabicyclo[3.1.0]hexan-2-yl)methyl)phenyl)urea. |
| | | | LCMS-ESI (POS.) m/z: 366.15 (M + H)⁺. ¹H NMR (400 |
| | | | MHz, DMSO-d₆) δ 8.44 (s, 1 H), 7.30 (d, J = 8.5 Hz, 2 H), |
| | | | 7.16 (d, J = 8.6 Hz, 2 H), 7.06 (d, J = 8.5 Hz, 2 H), 6.83 |
| | | | (d, J = 8.6 Hz, 2 H), 6.44 (t, J = 5.9 Hz, 1 H), 4.22 (q, J = |
| | | | 14.5 Hz, 2 H), 4.15 (d, J = 5.8 Hz, 2 H), 3.66 (s, 3 H), 2.88 |
| | | | (ddt, J = 6.9, 5.0, 1.7 Hz, 1 H), 2.63 (dd, J = 17.8, 7.3 Hz, |
| | | | 1 H), 2.17 (d, J = 17.8 Hz, 1 H), 1.35 (qd, J = 7.7, 4.7 Hz, |
| | | | 1 H), 0.66 (dt, J = 8.4, 5.2 Hz, 1 H), 0.00 (td, J = 5.1, 2.1 |
| | | | Hz, 1 H). |
| 530 | 4- (benzyloxy) aniline | 4-chloro benzyl isocyanate | 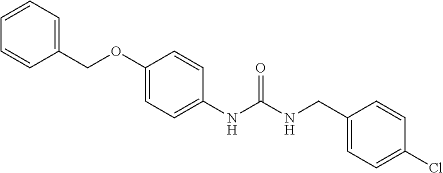 [SMILES: O=C(NCc1ccc(Cl)cc1)Nc1ccc(OCc2ccccc2)cc1] |
| | | | 1-(4-(benzyloxy)phenyl)-3-(4-chlorobenzyl)urea. |
| | | | LCMS-ESI (POS.) m/z: 367.10 (M + H)⁺. ¹H NMR (400 |
| | | | MHz, DMSO-d₆) δ 8.39 (s, 1 H), 7.43 (d, J = 7.0 Hz, 2 H), |
| | | | 7.41-7.35 (m, 4 H), 7.35-7.26 (m, 5 H), 6.89 (d, J = 8.9 |
| | | | Hz, 2 H), 6.55 (t, J = 6.0 Hz, 1 H), 5.03 (s, 2 H), 4.27 (d, J = |
| | | | 5.9 Hz, 2 H). |
| 531 | 4- (benzyloxy) aniline | 4-methoxy benzyl isocyanate | 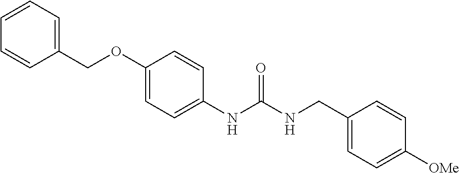 [SMILES: COc1ccc(CNC(=O)Nc2ccc(OCc3ccccc3)cc2)cc1] |
| | | | 1-(4-(benzyloxy)phenyl)-3-(4-chlorobenzyl)urea. |
| | | | LCMS-ESI (POS.) m/z: 363.15 (M + H)⁺. ¹H NMR (400 |
| | | | MHz, DMSO-d₆) δ 8.30 (s, 1 H), 7.43 (d, J = 6.7 Hz, 2 H), |
| | | | 7.38 (t, J = 7.4 Hz, 2 H), 7.35-7.26 (m, 3 H), 7.22 (d, J = |
| | | | 8.6 Hz, 2 H), 6.89 (d, J = 8.2 Hz, 4 H), 6.41 (t, J = 5.9 Hz, |
| | | | 1 H), 5.03 (s, 2 H), 4.20 (d, J = 5.8 Hz, 2 H), 3.73 (s, 3 H). |
| 532 | 4-(2- morpholino ethoxy) aniline | 4-chloro benzyl isocyanate | 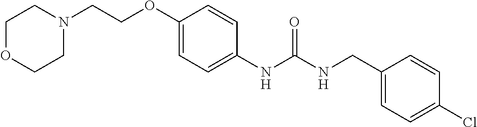 [SMILES: O=C(NCc1ccc(Cl)cc1)Nc1ccc(OCCN2CCOCC2)cc1] |
| | | | 1-(4-chlorobenzyl)-3-(4-(2- |
| | | | morpholinoethoxy)phenyl)urea. LCMS-ESI (POS.) |
| | | | m/z: 390.10 (M + H)⁺. ¹H NMR (400 MHz, DMSO-d₆) δ |
| | | | 8.38 (s, 1 H), 7.39 (d, J = 8.4 Hz, 2 H), 7.31 (d, J = 8.5 Hz, |
| | | | 2 H), 7.28 (d, J = 9.0 Hz, 2 H), 6.82 (d, J = 9.0 Hz, 2 H), |
| | | | 6.54 (t, J = 6.0 Hz, 1 H), 4.26 (d, J = 5.9 Hz, 2 H), 4.01 (t, |
| | | | J = 5.8 Hz, 2 H), 3.57 (t, J = 4.6 Hz, 4 H), 2.65 (t, J = 5.8 |
| | | | Hz, 2 H), 2.46 (t, J = 4.6 Hz, 4 H). |
| 533 | 4-((2- morpholino ethyl) sulfonyl) aniline | 4-chloro benzyl isocyanate | 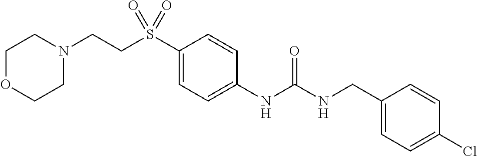 [SMILES: O=C(NCc1ccc(Cl)cc1)Nc1ccc(S(=O)(=O)CCN2CCOCC2)cc1] |
| | | | 1-(4-chlorobenzyl)-3-(4-((2- |
| | | | morpholinoethyl)sulfonyl)phenyl)urea. LCMS-ESI |
| | | | (POS.) m/z: 438.1 (M + H)⁺. ¹H NMR (400 MHz, DMSO- |
| | | | d₆) δ 9.22 (s, 1 H), 7.74 (d, J = 8.5 Hz, 2 H), 7.63 (d, J = |
| | | | 8.9 Hz, 2 H), 7.40 (d, J = 8.5 Hz, 2 H), 7.33 (d, J = 8.5 Hz, |
| | | | 2 H), 6.93 (t, J = 6.0 Hz, 1 H), 4.31 (d, J = 5.9 Hz, 2 H), |
| | | | 3.47-3.35 (m, 6 H), 2.54 (t, J = 7.6 Hz, 2 H), 2.25 (t, J = |
| | | | 4.5 Hz, 4 H). |
| 534 | 4- (isopropyl sulfonyl) aniline | 4-chloro benzyl isocyanate | 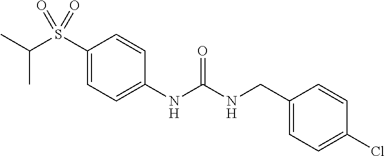 [SMILES: CC(C)S(=O)(=O)c1ccc(NC(=O)NCc2ccc(Cl)cc2)cc1] |
| | | | 1-(4-chlorobenzyl)-3-(4-(isopropylsulfonyl)phenyl)urea. |
| | | | LCMS-ESI (POS.) m/z: 367.00 (M + H)⁺. ¹H NMR (400 |
| | | | MHz, DMSO-d₆) δ 9.26 (s, 1 H), 7.68 (d, J = 9.1 Hz, 2 H), |
| | | | 7.65 (d, J = 9.2 Hz, 2 H), 7.40 (d, J = 8.5 Hz, 2 H), 7.33 |
| | | | (d, J = 8.5 Hz, 2 H), 6.95 (t, J = 6.0 Hz, 1 H), 4.31 (d, J = |
| | | | 5.9 Hz, 2 H), 3.29 (p, J = 6.8 Hz, 1 H), 1.13 (d, J = 6.8 Hz, |
| | | | 6 H). |
| 535 | 4- (cyclo propyl sulfonyl) aniline | 4-chloro benzyl isocyanate | 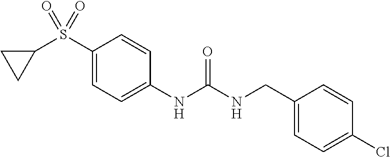 [SMILES: O=C(NCc1ccc(Cl)cc1)Nc1ccc(S(=O)(=O)C2CC2)cc1] |
| | | | 1-(4-chlorobenzyl)-3-(4- |
| | | | (cyclopropylsulfonyl)phenyl)urea. LCMS-ESI (POS.) |
| | | | m/z: 365.10 (M + H)⁺. ¹H NMR (400 MHz, DMSO-d₆) δ |
| | | | 9.24 (s, 1 H), 7.72 (d, J = 8.9 Hz, 2 H), 7.64 (d, J = 8.9 Hz, |
| | | | 2 H), 7.40 (d, J = 8.5 Hz, 2 H), 7.33 (d, J = 8.5 Hz, 2 H), |
| | | | 6.94 (t, J = 6.0 Hz, 1 H), 4.31 (d, J = 5.9 Hz, 2 H), 2.75 (tt, |
| | | | J = 7.9, 4.8 Hz, 1 H), 1.08-1.03 (m, 2H), 1.02-0.95 (m, 2 |
| | | | H). |

### Example 2

### Synthesis of ({4-[2-(3,3-difluoroazetidinyl)-2-oxoethyl]phenyl}amino)-N-[(4-methoxyphenyl)methyl]carboxamide (Compound 320)

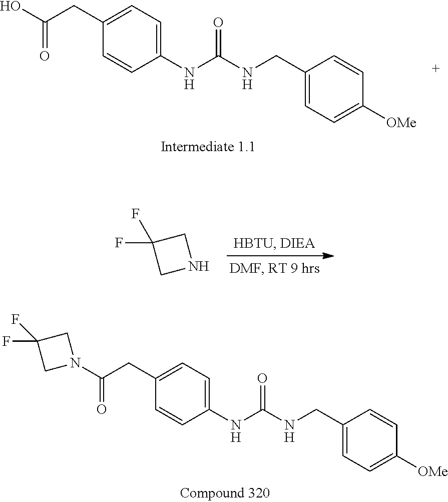 [SMILES: COc1ccc(CNC(=O)Nc2ccc(CC(=O)O)cc2)cc1 ; FC1(F)CNC1 ; COc1ccc(CNC(=O)Nc2ccc(CC(=O)N3CC(F)(F)C3)cc2)cc1]

To a room temperature solution of Intermediate 1.1 (100 mg, 0.318 mmol, 1.0 equiv), 3,3-difluoroazetidine (59 mg, 0.636 mmol, 2.0 equiv) and O-(benzotriazol-1-yl)-N,N,N′,N′-tetramethyluronium hexafluorophosphate (181 mg, 0.275 mmol, 1.5 equiv) in dimethylformamide (6 mL) was added N,N-diisopropylethylamine (0.006 mL, 0.03 mmol, 0.1 equiv). The resulting mixture was stirred at room temperature for approximately 9 hours. Resultant reaction mixture was diluted with water (0.5 mL) and extracted with ethyl acetate (2×1 mL). The organic phase was dried to a viscous oil which was purified by reverse phase HPLC with a 10%-100% acetonitrile in water solution that was run over 30 minutes in a Phenomonex Gemini 5u C18 column, providing Compound 320 (37.0 mg, 0.095 mmol, 29.9% yield) as a white foam. LCMS-APCI (POS.) m/z: 390.0 (M+H)+. 1H NMR (400 MHz, DMSO-d6) δ 8.46 (s, 1H), 7.37-7.29 (m, 2H), 7.27-7.18 (m, 2H), 7.12-7.04 (m, 2H), 6.94-6.85 (m, 2H), 6.49 (t, J=5.9 Hz, 1H), 4.61 (t, J=12.5 Hz, 2H), 4.33-4.18 (m, 4H), 3.73 (s, 3H).

Compounds in the following table were prepared in a similar manner as Compound 320, using the intermediates and reagents as listed.

| Ex # | Intermediate | Amine | Structure, Name and Data |
|---|---|---|---|
| 349 | 1.1 | ethyl amine | 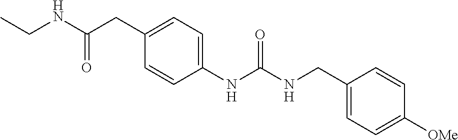 [SMILES: CCNC(=O)Cc1ccc(NC(=O)NCc2ccc(OC)cc2)cc1] |
| | | | Compound 349: N-ethyl-2-[4-({N-[(4- |
| | | | methoxyphenyl)methyl]carbamoyl}amino)phenyl] |
| | | | acetamide. LCMS-ESI (POS.) m/z: 342.1 (M + H)+. |
| | | | 1H NMR (400 MHz, DMSO-d6) δ 8.49 (s, 1H), 7.92 |
| | | | (t, J = 5.5 Hz, 1H), 7.34-7.25 (m, 2H), 7.25-7.16 |
| | | | (m, 2H), 7.13-7.01 (m, 2H), 6.94-6.84 (m, 2H), |
| | | | 6.55 (t, J = 5.9 Hz, 1H), 4.21 (d, J = 5.8 Hz, 2H), |
| | | | 3.73 (s, 3H), 3.27 (s, 2H), 3.05 (qd, J = 7.2, 5.4 Hz, |
| | | | 2H), 1.00 (t, J = 7.2 Hz, 3H). |
| 231 | 1.1 | piperidine | 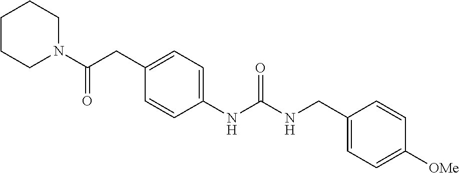 [SMILES: COc1ccc(CNC(=O)Nc2ccc(CC(=O)N3CCCCC3)cc2)cc1] |
| | | | Compound 231: N-[(4-methoxyphenyl)methyl]{[4- |
| | | | (2-oxo-2-piperidylethyl)phenyl]amino}carboxamide. |
| | | | LCMS-ESI (POS.) m/z: 382.1 (M + H)+. 1H NMR |
| | | | (400 MHz, DMSO-d6) δ 8.52 (s, 1H), 7.37-7.29 |
| | | | (m, 2H), 7.27-7.19 (m, 2H), 7.11-7.04 (m, 2H), |
| | | | 6.93-6.83 (m, 2H), 6.57 (t, J = 5.8 Hz, 1H), 4.21 (d, |
| | | | J = 5.8 Hz, 2H), 3.73 (s, 3H), 3.58 (s, 2H), 3.44- |
| | | | 3.38 (m, 4H), 1.52 (td, J = 6.9, 4.6 Hz, 2H), 1.38 (tq, |
| | | | J = 7.7, 4.9, 4.2 Hz, 2H), 1.30 (tq, J = 7.5, 5.0, 4.2 Hz, 2H). |
| 272 | 1.1 | cyclopen- tanamine | 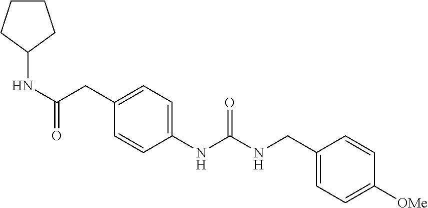 [SMILES: COc1ccc(CNC(=O)Nc2ccc(CC(=O)NC3CCCC3)cc2)cc1] |
| | | | Compound 272: N-cyclopentyl-2-[4-({N-[(4- |
| | | | methoxyphenyl)methyl]carbamoyl}amino)phenyl] |
| | | | acetamide. LCMS-ESI (POS.) m/z: 382.1 (M + H)+. |
| | | | 1H NMR (400 MHz, DMSO-d6) δ 8.48 (s, 1H), 7.93 |
| | | | (d, J = 7.3 Hz, 1H), 7.34-7.25 (m, 2H), 7.25-7.17 |
| | | | (m, 2H), 7.11-7.03 (m, 2H), 6.92-6.86 (m, 2H), |
| | | | 6.55 (t, J = 5.9 Hz, 1H), 4.21 (d, J = 5.8 Hz, 2H), |
| | | | 3.95 (h, J = 6.8 Hz, 1H), 3.73 (s, 3H), 3.26 (s, 2H), |
| | | | 1.77 (dddd, J = 13.9, 7.2, 5.4, 1.3 Hz, 2H), 1.66- |
| | | | 1.54 (m, 2H), 1.57-1.40 (m, 2H), 1.40-1.26 (m, 2H). |
| 347 | 1.1 | 3- methylaze- tidin-3-ol | 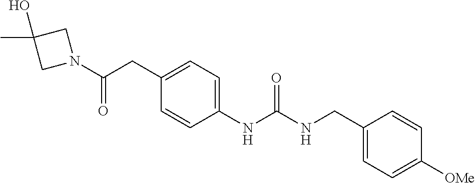 [SMILES: COc1ccc(CNC(=O)Nc2ccc(CC(=O)N3CC(C)(O)C3)cc2)cc1] |
| | | | Compound 347: ({4-[2-(3-hydroxy-3- |
| | | | methylazetidinyl)-2-oxoethyl]phenyl}amino)-N- |
| | | | [(4-methoxyphenyl)methyl]carboxamide. LCMS- |
| | | | ESI (POS.) m/z: 384.1 (M + H)+. 1H NMR (400 |
| | | | MHz, DMSO-d6) δ 8.50 (s, 1H), 7.37-7.26 (m, |
| | | | 2H), 7.26-7.18 (m, 2H), 7.11-7.02 (m, 2H), 6.93- |
| | | | 6.83 (m, 2H), 6.54 (t, J = 5.9 Hz, 1H), 5.63 (s, 1H), |
| | | | 4.21 (d, J = 5.8 Hz, 2H), 4.02-3.87 (m, 2H), 3.73 (s, |
| | | | 3H), 3.70-3.60 (m, 2H), 3.34-3.29 (m, 2H), 1.34 (s, 3H). |
| 350 | 1.1 | 3-methoxy- azetidine | 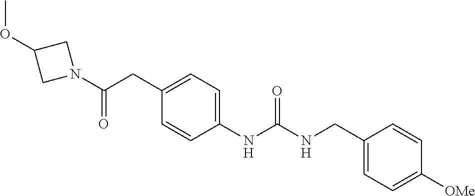 [SMILES: COc1ccc(CNC(=O)Nc2ccc(CC(=O)N3CC(OC)C3)cc2)cc1] |
| | | | Compound 350: ({4-[2-(3-methoxyazetidinyl)-2- |
| | | | oxoethyl]phenyl}amino)-N-[(4- |
| | | | methoxyphenyl)methyl]carboxamide. LCMS-ESI |
| | | | (POS.) m/z: 384.1 (M + H)+. 1H NMR (400 MHz, |
| | | | DMSO-d6) δ 8.50 (s, 1H), 7.35-7.26 (m, 2H), |
| | | | 7.26-7.18 (m, 2H), 7.10-7.02 (m, 2H), 6.95-6.84 (m, |
| | | | 2H), 6.54 (t, J = 5.9 Hz, 1H), 4.30 (ddd, J = 9.3, 6.4, |
| | | | 1.3 Hz, 1H), 4.21 (d, J = 5.8 Hz, 2H), 4.17 (s, 1H), |
| | | | 4.05-3.89 (m, 2H), 3.73 (s, 3H), 3.62 (ddd, J = |
| | | | 10.4, 3.9, 1.3 Hz, 1H), 3.33 (s, 2H), 3.20 (s, 3H). |
| 329 | 1.1 | (3-fluoro- azetidin- 3-yl) methanol | 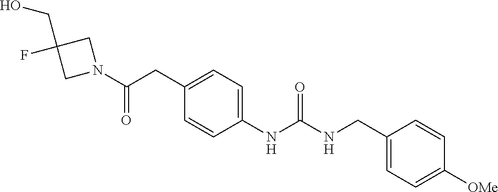 [SMILES: COc1ccc(CNC(=O)Nc2ccc(CC(=O)N3CC(F)(CO)C3)cc2)cc1] |
| | | | Compound 329: [(4-{2-[3-fluoro-3- |
| | | | (hydroxymethyl)azetidinyl]-2- |
| | | | oxoethyl}phenyl)amino]-N-[(4- |
| | | | methoxyphenyl)methyl]carboxamide. LCMS-ESI |
| | | | (POS.) m/z: 402.1 (M + H)+. 1H NMR (400 MHz, |
| | | | DMSO-d6) δ 8.57 (s, 1H), 7.38-7.27 (m, 2H), |
| | | | 7.26-7.17 (m, 2H), 7.13-7.02 (m, 2H), 6.94-6.84 (m, |
| | | | 2H), 6.61 (t, J = 5.9 Hz, 1H), 5.34 (s, 1H), 4.34- |
| | | | 4.10 (m, 4H), 3.97 (ddd, J = 18.2, 11.1, 1.5 Hz, 1H), |
| | | | 3.84 (ddd, J = 21.3, 11.1, 1.4 Hz, 1H), 3.73 (s, 3H), |
| | | | 3.65 (d, J = 21.0 Hz, 2H), 3.37 (s, 2H). |
| 310 | 1.1 | tert- butyl 3- amino- azetidine-1- carboxylate | 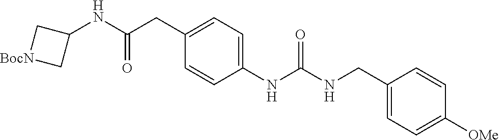 [SMILES: COc1ccc(CNC(=O)Nc2ccc(CC(=O)NC3CN(C(=O)OC(C)(C)C)C3)cc2)cc1] |
| | | | Compound 310: tert-butyl 3-{2-[4-({N-[(4- |
| | | | methoxyphenyl)methyl]carbamoyl}amino)phenyl] |
| | | | acetylamino}azetidinecarboxylate. LCMS-ESI |
| | | | (POS.) m/z: 369.1 (M-Boc + H)+. 1H NMR (400 |
| | | | MHz, DMSO-d6) δ 8.62 (d, J = 7.1 Hz, 1H), 8.47 (s, |
| | | | 1H), 7.38-7.26 (m, 2H), 7.26-7.15 (m, 2H), 7.14- |
| | | | 7.03 (m, 2H), 6.94-6.83 (m, 2H), 6.51 (t, J = 5.9 |
| | | | Hz, 1H), 4.37 (qt, J = 7.6, 5.3 Hz, 1H), 4.21 (d, J = |
| | | | 5.8 Hz, 2H), 4.04 (t, J = 8.2 Hz, 2H), 3.73 (s, 3H), |
| | | | 3.65 (dd, J = 8.7, 5.3 Hz, 2H), 3.31 (s, 2H), 1.38 (s, 9H). |
| 258 | 1.1 | 3,3- difluoro pyrrolidine | 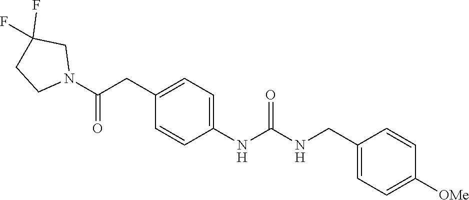 [SMILES: COc1ccc(CNC(=O)Nc2ccc(CC(=O)N3CCC(F)(F)C3)cc2)cc1] |
| | | | Compound 258: ({4-[2-(3,3-difluoropyrrolidinyl)- |
| | | | 2-oxoethyl]phenyl}amino)-N-[(4- |
| | | | methoxyphenyl)methyl]carboxamide. LCMS-ESI |
| | | | (POS.) m/z: 404.1 (M + H)+. 1H NMR (400 MHz, |
| | | | DMSO-d6) δ 8.46 (s, 1H), 7.36-7.27 (m, 2H), |
| | | | 7.25-7.19 (m, 2H), 7.13-7.02 (m, 2H), 6.96-6.83 (m, |
| | | | 2H), 6.50 (t, J = 5.9 Hz, 1H), 4.21 (d, J = 5.8 Hz, |
| | | | 2H), 3.95 (t, J = 13.1 Hz, 1H), 3.73 (s, 5H), 3.60-3.44 |
| | | | (m, 3H), 2.49-2.41 (m, 1H), 2.37 (dq, J = 14.5, 7.1 Hz, 1H). |
| 326 | 1.1 | 2-ethoxy- cyclo- propan-1- amine (1:1.3 mixture of diasatereo- mers) | 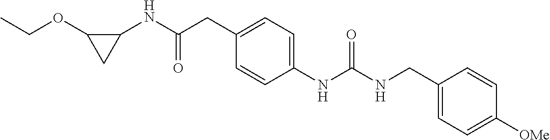 [SMILES: CCOC1CC1NC(=O)Cc1ccc(NC(=O)NCc2ccc(OC)cc2)cc1] |
| | | | Compound 326: N-(2-ethoxycyclopropyl)-2-[4- |
| | | | ({N-[(4-methoxyphenyl)methyl]carbamoyl}amino)phenyl] |
| | | | acetamide. LCMS-ESI (POS.) m/z: 398.1 (M + H)+. |
| | | | Diasteromer 1: 1H NMR (400 MHz, DMSO-d6) δ |
| | | | 8.44 (d, J = 1.3 Hz, 1H), 8.01 (d, J = 3.7 Hz, 1H), |
| | | | 7.34-7.25 (m, 2H), 7.25-7.16 (m, 2H), 7.13-7.01 |
| | | | (m, 2H), 6.96-6.83 (m, 2H), 6.48 (td, J = 5.8, 3.5 |
| | | | Hz, 1H), 4.21 (d, J = 5.8 Hz, 2H), 3.73 (s, 3H), |
| | | | 3.54-3.45 (m, 1H), 3.40-3.33 (m, 1H), 3.24-3.21 (s, |
| | | | 2H), 3.19-3.11 (m, 1H), 2.58-2.52 (m, 1H), 1.09 |
| | | | (t, J = 7.0 Hz, 2H), 0.93-0.81 (m, 2H), 0.68 (td, J = 6.7, 4.9 Hz, 1H). |
| | | | Diasteromer 2: 1H NMR (400 MHz, DMSO-d6) δ |
| | | | 8.44 (d, J = 1.3 Hz, 1H), 7.86 (d, J = 3.7 Hz, 1H), |
| | | | 7.34-7.25 (m, 2H), 7.25-7.16 (m, 2H), 7.13-7.01 |
| | | | (m, 2H), 6.96-6.83 (m, 2H), 6.48 (td, J = 5.8, 3.5 |
| | | | Hz, 1H), 4.21 (d, J = 5.8 Hz, 2H), 3.73 (s, 3H), |
| | | | 3.62-3.54 (m, 1H), 3.40-3.33 (m, 1H), 3.24-3.21 (s, |
| | | | 2H), 3.11-3.04 (m, 1H), 2.69-2.59 (m, 1H), 1.06 |
| | | | (t, J = 7.0 Hz, 2H), 0.93-0.81 (m, 2H), 0.58 (td, J = 6.7, 4.9 Hz, 1H). |
| 325 | 1.1 | tert- butyl (R)- pyrrolidin- 3-yl- carbamate | 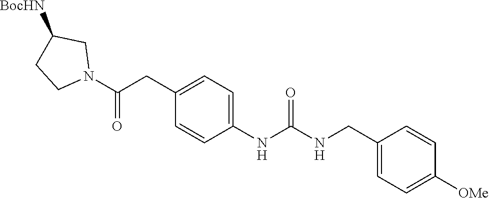 [SMILES: COc1ccc(CNC(=O)Nc2ccc(CC(=O)N3CC[C@@H](NC(=O)OC(C)(C)C)C3)cc2)cc1] |
| | | | Compound 325: {[4-(2-{(3R)-3-[(tert- |
| | | | butoxy)carbonylamino]pyrrolidinyl}-2- |
| | | | oxoethyl)phenyl]amino}-N-[(4- |
| | | | methoxyphenyl)methyl]carboxamide. LCMS-ESI |
| | | | (POS.) m/z: 383.1 (M-Boc + H)+. 1H NMR (400 |
| | | | MHz, DMSO-d6) δ 8.45 (d, J = 1.9 Hz, 1H), 7.35- |
| | | | 7.26 (m, 2H), 7.26-7.18 (m, 2H), 7.15 (t, J = 8.6 |
| | | | Hz, 1H), 7.10-7.02 (m, 2H), 6.93-6.84 (m, 2H), |
| | | | 6.49 (td, J = 5.9, 1.9 Hz, 1H), 4.21 (d, J = 5.8 Hz, |
| | | | 2H), 3.93 (t, J = 7.6 Hz, 1H), 3.73 (s, 3H), 3.67- |
| | | | 3.51 (m, 1H), 3.50-3.34 (m, 4H), 3.26 (ddd, J = |
| | | | 15.4, 9.0, 3.3 Hz, 1H), 1.94 (ddt, J = 12.5, 7.9, 6.3 |
| | | | Hz, 1H), 1.85-1.64 (m, 1H), 1.39 (d, J = 2.5 Hz, 9H). |
| 224 | 1.1 | octahydro- cyclopenta [c]pyrrole | 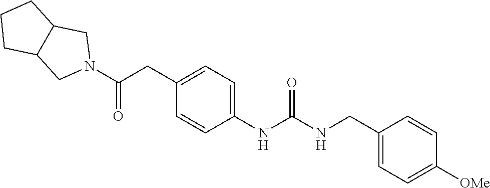 [SMILES: COc1ccc(CNC(=O)Nc2ccc(CC(=O)N3CC4CCCC4C3)cc2)cc1] |
| | | | Compound 224: ({4-[2-(3-azabicyclo[3.3.0]oct-3- |
| | | | yl)-2-oxoethyl]phenyl}amino)-N-[(4- |
| | | | methoxyphenyl)methyl]carboxamide. LCMS-ESI |
| | | | (POS.) m/z: 408.1 (M + H)+. 1H NMR (400 MHz, |
| | | | DMSO-d6) δ 8.44 (s, 1H), 7.33-7.27 (m, 2H), |
| | | | 7.27-7.17 (m, 2H), 7.12-7.02 (m, 2H), 6.94-6.84 (m, |
| | | | 2H), 6.49 (t, J = 5.9 Hz, 1H), 4.21 (d, J = 5.6 Hz, |
| | | | 2H), 3.73 (s, 3H), 3.64-3.58 (m, 1H), 3.54-3.43 |
| | | | (m, 3H), 3.23 (dd, J = 10.8, 4.6 Hz, 1H), 3.11 (dd, |
| | | | J = 12.2, 4.8 Hz, 1H), 2.65-2.52 (m, 2H), 1.83-1.24 (m, 6H). |
| 252 | 1.1 | tetrahydro- 2H-pyran- 3-amine | 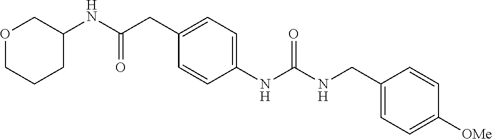 [SMILES: COc1ccc(CNC(=O)Nc2ccc(CC(=O)NC3CCCOC3)cc2)cc1] |
| | | | Compound 252: N-(2H-3,4,5,6-tetrahydropyran- |
| | | | 3-yl)-2-[4-({N-[(4- |
| | | | methoxyphenyl)methyl]carbamoyl}amino)phenyl] |
| | | | acetamide. LCMS-ESI (POS.) m/z: 398.1 (M + H)+. |
| | | | Diasteromer 1: 1H NMR (400 MHz, DMSO-d6) δ |
| | | | 8.44 (s, 1H), 7.95 (d, J = 7.4 Hz, 1H), 7.37-7.26 (m, |
| | | | 2H), 7.25-7.16 (m, 2H), 7.14-7.03 (m, 2H), 6.95- |
| | | | 6.82 (m, 2H), 6.50 (t, J = 5.9 Hz, 1H), 4.21 (d, J = |
| | | | 5.8 Hz, 2H), 3.73 (s, 3H), 3.65 (tdd, J = 9.5, 4.5, 2.7 |
| | | | Hz, 3H), 3.37-3.31 (m, 1H), 3.31 (s, 2H), 3.13-3.02 |
| | | | (m, 1H), 1.84-1.73 (m, 1H), 1.71-1.58 (m, 1H), |
| | | | 1.56-1.35 (m, 2H). |
| 244 | 1.1 | cyclopropyl- methanamine | 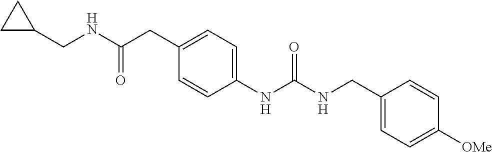 [SMILES: COc1ccc(CNC(=O)Nc2ccc(CC(=O)NCC3CC3)cc2)cc1] |
| | | | Compound 244: N-(cyclopropylmethyl)-2-[4-({N-[(4- |
| | | | methoxyphenyl)methyl]carbamoyl}amino)phenyl] |
| | | | acetamide. LCMS-ESI (POS.) m/z: 368.1 (M + H)+. |
| | | | 1H NMR (400 MHz, DMSO-d6) δ 8.31 (s, 1H), 7.90 |
| | | | (t, J = 5.6 Hz, 1H), 7.25-7.14 (m, 2H), 7.14-7.03 |
| | | | (m, 2H), 7.03-6.91 (m, 2H), 6.83-6.71 (m, 2H), |
| | | | 6.36 (t, J = 5.9 Hz, 1H), 4.09 (d, J = 5.8 Hz, 2H), |
| | | | 3.61 (s, 3H), 3.18 (s, 2H), 2.80 (dd, J = 6.8, 5.5 Hz, |
| | | | 2H), 0.83-0.66 (m, 1H), 0.30-0.16 (m, 2H). 0.02-(-0.04)) (m, 2H). |
| 346 | 1.1 | (R)- pyrrolidin- 3-ol | 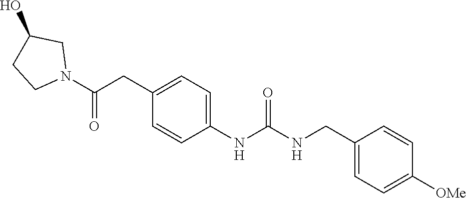 [SMILES: COc1ccc(CNC(=O)Nc2ccc(CC(=O)N3CC[C@@H](O)C3)cc2)cc1] |
| | | | Compound 346: ({4-[2-((3R)-3- |
| | | | hydroxypyrrolidinyl)-2-oxoethyl]phenyl}amino)- |
| | | | N-[(4-methoxyphenyl)methyl]carboxamide. |
| | | | LCMS-ESI (POS.) m/z: 384.1 (M + H)+. 1H NMR |
| | | | (400 MHz, DMSO-d6) δ 8.46 (s, 1H), 7.35-7.29 |
| | | | (m, 2H), 7.22 (d, J = 8.7 Hz, 2H), 7.11-7.03 (m, |
| | | | 2H), 6.93-6.86 (m, 2H), 6.51 (t, J = 5.9 Hz, 1H), 4.99 |
| | | | (d, J = 3.7 Hz, 1H), 4.29 (s, 1H), 4.21 (d, J = 5.9 Hz, 2H), |
| | | | 3.73 (s, 3H), 3.52 (m, 4H), 3.29-3.20 (m, 2H), 1.77 (m, 2H). |
| 202 | 1.1 | (4-fluoro- phenyl) (piperazin- 1-yl) methanone | 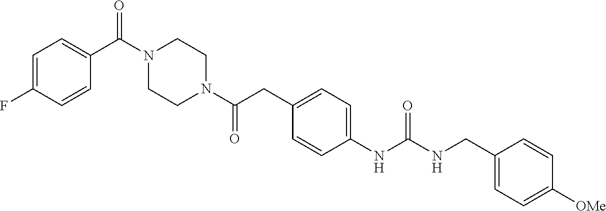 [SMILES: COc1ccc(CNC(=O)Nc2ccc(CC(=O)N3CCN(C(=O)c4ccc(F)cc4)CC3)cc2)cc1] |
| | | | Compound 202: {[4-(2-{4-[(4- |
| | | | fluorophenyl)carbonyl]piperazinyl}-2- |
| | | | oxoethyl)phenyl]amino}-N-[(4- |
| | | | methoxyphenyl)methyl]carboxamide. LCMS-ESI |
| | | | (POS.) m/z: 505.1 (M + H)+. 1H NMR (400 MHz, |
| | | | DMSO-d6) δ 8.46 (s, 1H), 7.52-7.43 (m, 2H), 7.36-7.18 |
| | | | (m, 6H), 7.08 (d, J = 8.1 Hz, 2H), 6.93-6.84 |
| | | | (m, 2H), 6.50 (t, J = 5.9 Hz, 1H), 4.21 (d, J = 5.8 Hz, |
| | | | 2H), 3.73 (s, 3H), 3.64 (s, 2H), 3.52 (br s, 8H). |
| 241 | 1.1 | 3-phenyl- cyclopentan- 1-amine | 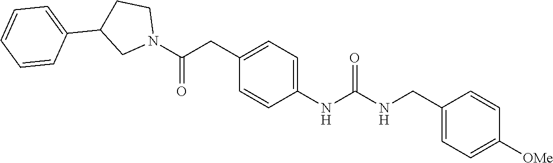 [SMILES: COc1ccc(CNC(=O)Nc2ccc(CC(=O)N3CCC(c4ccccc4)C3)cc2)cc1] |
| | | | Compound 241: 2-[4-({N-[(4- |
| | | | methoxyphenyl)methyl]carbamoyl}amino)phenyl]- |
| | | | N-(3-phenylcyclopentyl)acetamide. LCMS-ESI |
| | | | (POS.) m/z: 458.1 (M + H)+. 1H NMR (400 MHz, |
| | | | DMSO-d6) δ 8.44 (s, 1H), 8.12 (dd, J = 7.3, 5.6 Hz, |
| | | | 1H), 7.36-7.13 (m, 9H), 7.13-7.06 (m, 2H), 6.94- |
| | | | 6.84 (m, 2H), 6.49 (t, J = 5.8 Hz, 1H), 4.21 (d, J = |
| | | | 5.8 Hz, 2H), 3.73 (s, 3H), 3.30 (s, 2H), 3.01 (tt, J = |
| | | | 10.8, 7.1 Hz, 1H), 2.30 (dt, J = 13.6, 7.1 Hz, 1H), |
| | | | 2.10 (td, J = 10.6, 8.9, 4.4 Hz, 1H), 2.01-1.89 (m, 1H), |
| | | | 1.88-1.80 (m, 1H), 1.74-1.62 (m, 1H), 1.62-1.38 (m, 2H). |
| 335 | 1.1 | 3- (methoxy- methyl) azetidine | 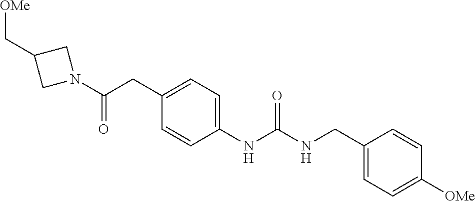 [SMILES: COCC1CN(C(=O)Cc2ccc(NC(=O)NCc3ccc(OC)cc3)cc2)C1] |
| | | | Compound 335: [(4-{2-[3- |
| | | | (methoxymethyl)azetidinyl]-2- |
| | | | oxoethyl}phenyl)amino]-N-[(4- |
| | | | methoxyphenyl)methyl]carboxamide. LCMS-ESI |
| | | | (POS.) m/z: 398.1 (M + H)+. 1H NMR (400 MHz, |
| | | | DMSO-d6) δ 8.49 (s, 1H), 7.35-7.27 (m, 2H), |
| | | | 7.25-7.18 (m, 2H), 7.09-7.00 (m, 2H), 6.95-6.83 (m, |
| | | | 2H), 6.54 (t, J = 5.9 Hz, 1H), 4.21 (d, J = 5.8 Hz, |
| | | | 2H), 4.16 (t, J = 8.4 Hz, 1H), 3.90-3.81 (m, 2H), |
| | | | 3.73 (s, 3H), 3.57-3.50 (m, 1H), 3.44 (d, J = 6.5 Hz, |
| | | | 2H), 3.30 (s, 2H), 3.26 (s, 3H), 2.82-2.69 (m, 1H). |
| 286 | 1.1 | 4- (azetidin- 3-yl) pyridine | 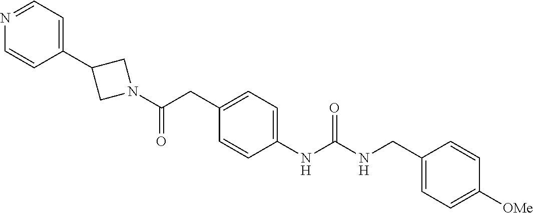 [SMILES: COc1ccc(CNC(=O)Nc2ccc(CC(=O)N3CC(c4ccncc4)C3)cc2)cc1] |
| | | | Compound 286: N-[(4-methoxyphenyl)methyl]({4-[2-oxo-2-(3-(4- |
| | | | pyridyl)azetidinyl)ethyl]phenyl}amino)carboxamide. |
| | | | LCMS-ESI (POS.) m/z: 431.1 (M + H)+. 1H |
| | | | NMR (400 MHz, DMSO-d6) δ 8.60-8.50 (m, 2H), |
| | | | 8.46 (s, 1H), 7.38-7.26 (m, 4H), 7.25-7.17 (m, |
| | | | 2H), 7.14-7.04 (m, 2H), 6.95-6.83 (m, 2H), 6.49 |
| | | | (t, J = 5.9 Hz, 1H), 4.55 (t, J = 8.3 Hz, 1H), 4.31- |
| | | | 4.14 (m, 4H), 3.87 (q, J = 6.0 Hz, 2H), 3.73 (s, 3H), 3.38 (s, 2H). |
| 284 | 1.1 | 3,3- dimethyl- azetidine | 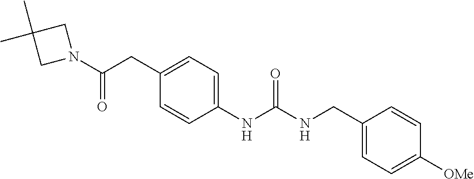 [SMILES: COc1ccc(CNC(=O)Nc2ccc(CC(=O)N3CC(C)(C)C3)cc2)cc1] |
| | | | Compound 284: ({4-[2-(3,3-dimethylazetidinyl)-2- |
| | | | oxoethyl]phenyl}amino)-N-[(4- |
| | | | methoxyphenyl)methyl]carboxamide. LCMS-ESI |
| | | | (POS.) m/z: 382.1 (M + H)+. 1H NMR (400 MHz, |
| | | | DMSO-d6) δ 8.46 (s, 1H), 7.35-7.26 (m, 2H), |
| | | | 7.26-7.17 (m, 2H), 7.11-7.01 (m, 2H), 6.95-6.82 (m, 2H), |
| | | | 6.51 (t, J = 5.9 Hz, 1H), 4.21 (d, J = 5.8 Hz, 2H), 3.80 |
| | | | (s, 2H), 3.73 (s, 3H), 3.47 (s, 2H), 3.31 (s, 2H), 1.20 (s, 6H). |
| 308 | 1.1 | tert-butyl (3S,4S)- 3-amino- 4-methoxy- pyrrolidine- 1-carboxylate | 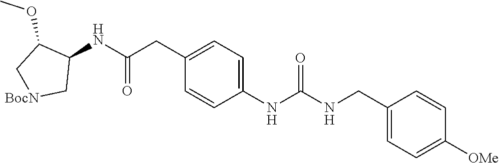 [SMILES: COc1ccc(CNC(=O)Nc2ccc(CC(=O)N[C@H]3CN(C(=O)OC(C)(C)C)C[C@@H]3OC)cc2)cc1] |
| | | | Compound 308: tert-butyl (3S,4S)-3-methoxy-4- |
| | | | (2-[4-({N-[(4- |
| | | | methoxyphenyl)methyl]carbamoyl}amino)phenyl] |
| | | | acetylamino}pyrrolidinecarboxylate. LCMS-ESI |
| | | | (POS.) m/z: 413.0 (M-Boc + H)+. 1H NMR (400 |
| | | | MHz, DMSO-d6) δ 8.45 (s, 1H), 8.31 (d, J = 6.8 Hz, |
| | | | 1H), 7.37-7.26 (m, 2H), 7.26-7.17 (m, 2H), 7.14- |
| | | | 7.04 (m, 2H), 6.95-6.84 (m, 2H), 6.49 (t, J = 5.9 |
| | | | Hz, 1H), 4.21 (d, J = 5.8 Hz, 2H), 4.12 (d, J = 8.1 Hz, |
| | | | 1H), 3.73 (s, 3H), 3.64 (d, J = 3.4 Hz, 1H), 3.42 (ddd, |
| | | | J = 16.4, 11.8, 5.3 Hz, 2H), 3.33 (s, 2H), 3.30-3.27 (m, 1H), |
| | | | 3.26 (s, 3H), 3.20-3.05 (m, 1H), 1.41 (d, J = 4.2 Hz, 9H). |
| 353 | 1.1 | azetidine | 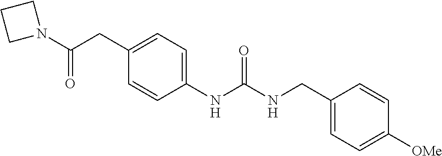 [SMILES: COc1ccc(CNC(=O)Nc2ccc(CC(=O)N3CCC3)cc2)cc1] |
| | | | Compound 353: {[4-(2-azetidinyl-2- |
| | | | oxoethyl)phenyl]amino}-N-[(4- |
| | | | methoxyphenyl)methyl]carboxamide. LCMS-ESI |
| | | | (POS.) m/z: 354.1 (M + H)+. 1H NMR (400 MHz, |
| | | | DMSO-d6) δ 8.53 (s, 1H), 7.37-7.27 (m, 2H), |
| | | | 7.25-7.15 (m, 2H), 7.11-7.00 (m, 2H), 6.96-6.81 (m, |
| | | | 2H), 6.57 (t, J = 5.9 Hz, 1H), 4.21 (d, J = 5.8 Hz, |
| | | | 2H), 4.17-4.06 (m, 2H), 3.82 (t, J = 7.7 Hz, 2H), |
| | | | 3.73 (s, 3H), 3.28 (s, 2H), 2.24-2.01 (m, 2H). |
| 341 | 1.1 | (R)- (tetrahydro- furan-3-yl) methanamine | 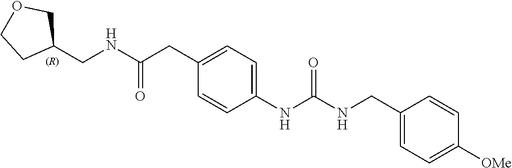 |
| | | | Compound 341: N-[((3R)oxolan-3-yl)methyl]-2-[4-({N-[(4- |
| | | | methoxyphenyl)methyl]carbamoyl}amino)phenyl] |
| | | | acetamide. LCMS-ESI (POS.) m/z: 398.1 (M + H)+. |
| | | | 1H NMR (400 MHz, DMSO-d6) δ 8.43 (s, 1H), 8.07 |
| | | | (t, J = 5.7 Hz, 1H), 7.34-7.26 (m, 2H), 7.26-7.15 |
| | | | (m, 2H), 7.14-7.02 (m, 2H), 6.94-6.82 (m, 2H), |
| | | | 6.48 (t, J = 5.9 Hz, 1H), 4.21 (d, J = 5.8 Hz, 2H), |
| | | | 3.73 (s, 3H), 3.70-3.51 (m, 4H), 3.30 (s, 2H), 3.11-2.95 |
| | | | (m, 2H), 2.37-2.20 (m, 1H), 1.87 (dtd, J = 12.3, 7.9, |
| | | | 5.6 Hz, 1H), 1.49 (dddd, J = 12.5, 7.9, 6.7, 5.9 Hz, 1H). |
| 135 | 1.2 | ethylamine | 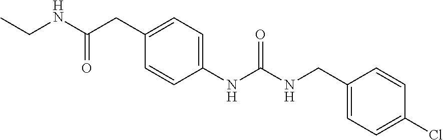 [SMILES: CCNC(=O)Cc1ccc(NC(=O)NCc2ccc(Cl)cc2)cc1] |
| | | | Compound 135: 2-[4-({N-[(4- |
| | | | chlorophenyl)methyl]carbamoyl}amino)phenyl]- |
| | | | N-ethylacetamide. LCMS-ESI (POS.) m/z: 346.0 |
| | | | (M + H)+. 1H NMR (400 MHz, DMSO-d6) δ 8.84 (s, |
| | | | 1H), 7.92 (t, J = 5.5 Hz, 1H), 7.43-7.34 (m, 2H), |
| | | | 7.34-7.25 (m, 4H), 7.13-7.02 (m, 2H), 6.97 (s, |
| | | | 1H), 4.27 (d, J = 5.8 Hz, 2H), 3.27 (s, 2H), 3.05 (qd, |
| | | | J = 7.2, 5.4 Hz, 2H), 1.00 (t, J = 7.2 Hz, 3H). |
| 71 | 1.2 | diethylamine | 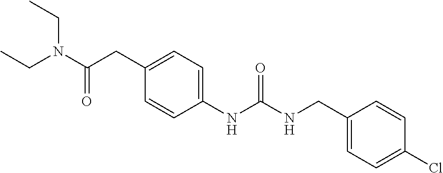 [SMILES: CCN(CC)C(=O)Cc1ccc(NC(=O)NCc2ccc(Cl)cc2)cc1] |
| | | | Compound 71: 2-[4-({N-[(4-2-[4-({N-[(4- |
| | | | chlorophenyl)methyl]carbamoyl}amino)phenyl]- |
| | | | N,N-diethylacetamide. LCMS-ESI (POS.) m/z: |
| | | | 374.0 (M + H)+. 1H NMR (400 MHz, DMSO-d6) δ |
| | | | 8.57 (s, 1H), 7.42-7.36 (m, 2H), 7.36-7.27 (m, |
| | | | 4H), 7.13-7.01 (m, 2H), 6.66 (t, J = 6.1 Hz, 1H), |
| | | | 4.28 (d, J = 5.9 Hz, 2H), 3.56 (s, 2H), 3.32-3.15 (m, |
| | | | 4H), 1.01 (dt, J = 8.3, 7.0 Hz, 6H). |
| 91 | 1.2 | cyclopenta- namine | 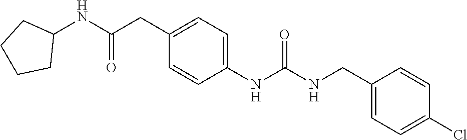 [SMILES: O=C(Cc1ccc(NC(=O)NCc2ccc(Cl)cc2)cc1)NC1CCCC1] |
| | | | Compound 91: 2-[4-({N-[(4-2-[4-({N-[(4- |
| | | | chlorophenyl)methyl]carbamoyl}amino)phenyl]- |
| | | | N-cyclopentylacetamide. LCMS-ESI (POS.) m/z: |
| | | | 386.0 (M + H)+. 1H NMR (400 MHz, DMSO-d6) δ |
| | | | 8.53 (s, 1H), 7.93 (d, J = 7.3 Hz, 1H), 7.44-7.34 (m, |
| | | | 2H), 7.35-7.23 (m, 4H), 7.16-7.03 (m, 2H), 6.63 |
| | | | (t, J = 6.0 Hz, 1H), 4.27 (d, J = 5.9 Hz, 2H), 3.95 (h, |
| | | | J = 6.9 Hz, 1H), 3.31 (s, 2H), 1.77 (dtdd, J = 12.6, |
| | | | 7.1, 5.4, 1.5 Hz, 2H), 1.68-1.53 (m, 2H), 1.53- |
| | | | 1.40 (m, 2H), 1.35 (dddd, J = 14.9, 12.2, 6.1, 1.7 Hz, 2H). |
| 138 | 1.2 | 2-(3-methyl- 1,2,4- oxadiazol-5- yl)ethana- mine | 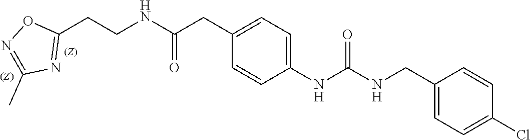 [SMILES: Cc1noc(CCNC(=O)Cc2ccc(NC(=O)NCc3ccc(Cl)cc3)cc2)n1] |
| | | | Compound 138: 2-[4-({N-[(4- |
| | | | chlorophenyl)methyl]carbamoyl}amino)phenyl]- |
| | | | N-[2-(3-methyl(1,2,4-oxadiazol-5- |
| | | | yl))ethyl]acetamide. LCMS-ESI (POS.) m/z: 428.0 |
| | | | (M + H)+. 1H NMR (400 MHz, DMSO-d6) δ 8.56 (s, |
| | | | 1H), 8.16 (t, J = 5.7 Hz, 1H), 7.44-7.35 (m, 2H), |
| | | | 7.35-7.25 (m, 4H), 7.12-7.00 (m, 2H), 6.64 (t, J = |
| | | | 6.0 Hz, 1H), 4.28 (d, J = 5.9 Hz, 2H), 3.50-3.38 (m, |
| | | | 2H), 3.28 (s, 2H), 3.02 (t, J = 6.8 Hz, 2H), 2.30 (s, 3H). |
| 64 | 1.2 | 3-methylaze- tidin-3-ol | 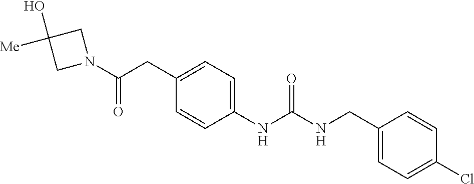 [SMILES: CC1(O)CN(C(=O)Cc2ccc(NC(=O)NCc3ccc(Cl)cc3)cc2)C1] |
| | | | Compound 64: N-[(4-chlorophenyl)methyl]({4-[2- |
| | | | (3-hydroxy-3-methylazetidinyl)-2- |
| | | | oxoethyl]phenyl}amino)carboxamide. LCMS-ESI |
| | | | (POS.) m/z: 388.0 (M + H)+. 1H NMR (400 MHz, |
| | | | DMSO-d6) δ 8.74 (s, 1H), 7.43-7.24 (m, 6H), 7.12-7.00 |
| | | | (m, 2H), 6.85 (t, J = 6.0 Hz, 1H), 5.76 (s, 1H), |
| | | | 4.27 (d, J = 5.8 Hz, 2H), 4.02-3.90 (m, 2H), 3.74-3.59 |
| | | | (m, 2H), 3.33 (d, J = 4.1 Hz, 2H), 1.34 (s, 3H). |
| 144 | 1.2 | ethanolamine | 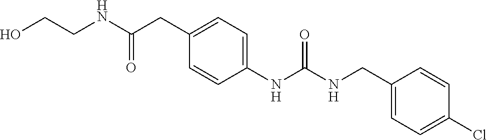 [SMILES: O=C(Cc1ccc(NC(=O)NCc2ccc(Cl)cc2)cc1)NCCO] |
| | | | Compound 144. 2-[4-({N-[(4- |
| | | | chlorophenyl)methyl]carbamoyl}amino)phenyl]- |
| | | | N-(2-hydroxyethyl)acetamide. LCMS-ESI (POS.) |
| | | | m/z: 362.0 (M + H)+. 1H NMR (400 MHz, DMSO- |
| | | | d6) δ 8.56 (s, 1H), 7.95 (t, J = 5.7 Hz, 1H), 7.42- |
| | | | 7.35 (m, 2H), 7.35-7.25 (m, 4H), 7.15-7.05 (m, 2H), |
| | | | 6.66 (s, 1H), 4.67 (s, 1H), 4.30-4.20 (m, 2H), 3.39 |
| | | | (t, J = 6.2 Hz, 2H), 3.31 (s, 2H), 3.10 (q, J = 5.8 Hz, 2H). |
| 119 | 1.2 | 3-methoxya- zetidine | 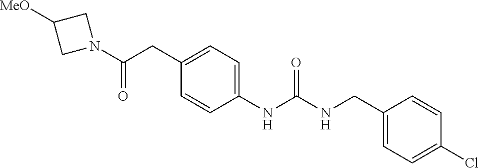 [SMILES: COC1CN(C(=O)Cc2ccc(NC(=O)NCc3ccc(Cl)cc3)cc2)C1] |
| | | | Compound 119. N-[(4-chlorophenyl)methyl]({4- |
| | | | [2-(3-methoxyazetidinyl)-2- |
| | | | oxoethyl]phenyl}amino)carboxamide. LCMS-ESI |
| | | | (POS.) m/z: 388.0 (M + H)+. 1H NMR (400 MHz, |
| | | | DMSO-d6) δ 8.56 (s, 1H), 7.42-7.35 (m, 2H), |
| | | | 7.35-7.26 (m, 4H), 7.13-7.00 (m, 2H), 6.64 (t, J = 6.0 |
| | | | Hz, 1H), 4.33-4.22 (m, 3H), 4.17 (tt, J = 6.4, 3.9 |
| | | | Hz, 1H), 4.04-3.87 (m, 2H), 3.62 (ddd, J = 10.4, |
| | | | 3.9, 1.3 Hz, 1H), 3.33 (s, 2H), 3.20 (s, 3H). |
| 105 | 1.2 | (3-fluoro- azetidin-3- yl)methanol | 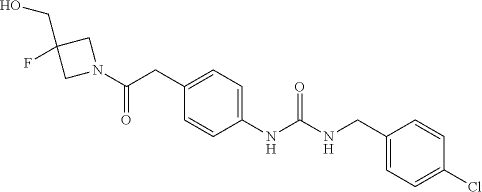 [SMILES: O=C(NCc1ccc(Cl)cc1)Nc1ccc(CC(=O)N2CC(F)(CO)C2)cc1] |
| | | | Compound 105. N-[(4-chlorophenyl)methyl][(4- |
| | | | {2-[3-fluoro-3-(hydroxymethyl)azetidinyl]-2- |
| | | | oxoethyl}phenyl)amino]carboxamide. LCMS-ESI |
| | | | (POS.) m/z: 406.0 (M + H)+. 1H NMR (400 MHz, |
| | | | DMSO-d6) δ 8.61 (s, 1H), 7.45-7.35 (m, 2H), |
| | | | 7.35-7.26 (m, 4H), 7.12-7.03 (m, 2H), 6.69 (t, J = 6.0 |
| | | | Hz, 1H), 5.37 (s, 1H), 4.34-4.25 (m, 3H), 4.25- |
| | | | 4.11 (m, 1H), 3.97 (ddd, J = 18.2, 11.1, 1.5 Hz, 1H), 3.84 |
| | | | (ddd, J = 21.2, 11.1, 1.4 Hz, 1H), 3.65 (d, J = 21.0 Hz, 2H), 3.38 (s, 2H). |
| 49 | 1.2 | Piperidine | 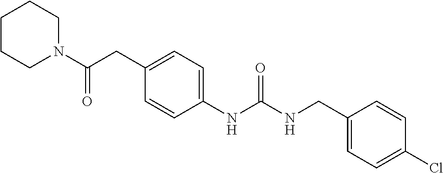 [SMILES: O=C(NCc1ccc(Cl)cc1)Nc1ccc(CC(=O)N2CCCCC2)cc1] |
| | | | Compound 49. N-[(4-chlorophenyl)methyl]{[4-(2- |
| | | | oxo-2-piperidylethyl)phenyl]amino}carboxamide. |
| | | | LCMS-ESI (POS.) m/z: 386.0 (M + H)+. 1H NMR |
| | | | (400 MHz, DMSO-d6) δ 8.58 (s, 1H), 7.44-7.36 |
| | | | (m, 2H), 7.36-7.26 (m, 4H), 7.12-6.99 (m, 2H), |
| | | | 6.65 (t, J = 6.0 Hz, 1H), 4.28 (d, J = 5.9 Hz, 2H), |
| | | | 3.59 (s, 2H), 3.40 (dt, J = 10.7, 5.4 Hz, 4H), 1.52 (td, |
| | | | J = 7.5, 7.0, 4.6 Hz, 2H), 1.38 (tq, J = 8.0, 5.1, 4.2 |
| | | | Hz, 2H), 1.30 (tq, J = 7.6, 4.9, 4.2 Hz, 2H). |
| 113 | 1.2 | (R)- pyrrolidin- 3-ol | 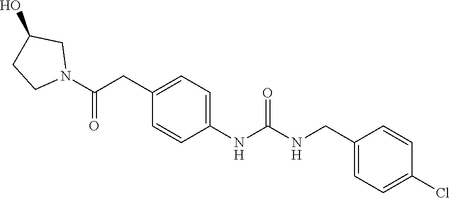 [SMILES: O=C(NCc1ccc(Cl)cc1)Nc1ccc(CC(=O)N2CC[C@@H](O)C2)cc1] |
| | | | Compound 113. ({4-[2-((3R)-3- |
| | | | hydroxypyrrolidinyl)-2-oxoethyl]phenyl}amino)- |
| | | | N-[(4-chlorophenyl)methyl]carboxamide. LCMS- |
| | | | ESI (POS.) m/z: 388.0 (M + H)+. 1H NMR (400 |
| | | | MHz, DMSO-d6) δ 8.55 (d, J = 1.9 Hz, 1H), 7.43- |
| | | | 7.35 (m, 2H), 7.35-7.25 (m, 4H), 7.08 (dd, J = 8.4, |
| | | | 1.5 Hz, 2H), 6.69-6.56 (m, 1H), 4.94 (dd, J = 40.2, |
| | | | 3.5 Hz, 1H), 4.28 (d, J = 6.0 Hz, 2H), 4.22 (tt, J = |
| | | | 4.5, 2.4 Hz, 1H), 3.59-3.45 (m, 3H), 3.40 (ddd, J = |
| | | | 12.1, 8.5, 3.8 Hz, 1H), 3.34-3.24 (m, 2H), 2.00-1.61 (m, 2H). |
| 131 | 1.2 | tert-butyl 3-aminoaze- tidine-1- carboxylate | 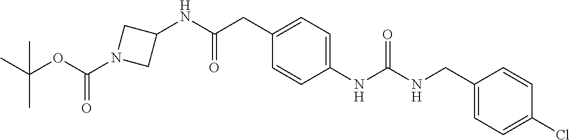 [SMILES: CC(C)(C)OC(=O)N1CC(NC(=O)Cc2ccc(NC(=O)NCc3ccc(Cl)cc3)cc2)C1] |
| | | | Compound 131. tert-butyl 3-{2-[4-({N-[(4- |
| | | | chlorophenyl)methyl]carbamoyl}amino)phenyl] |
| | | | acetylamino}azetidinecarboxylate. LCMS-ESI |
| | | | (POS.) m/z: 373.0 (M-Boc + H)+. 1H NMR (400 |
| | | | MHz, DMSO-d6) δ 8.66-8.56 (m, 2H), 7.41-7.34 |
| | | | (m, 2H), 7.34-7.25 (m, 4H), 7.14-7.03 (m, 2H), |
| | | | 6.67 (t, J = 6.0 Hz, 1H), 4.37 (dtd, J = 12.9, 7.5, 5.3 |
| | | | Hz, 1H), 4.28 (d, J = 6.0 Hz, 2H), 4.04 (t, J = 8.3 Hz, |
| | | | 2H), 3.65 (dd, J = 8.7, 5.4 Hz, 2H), 3.31 (s, 2H), 1.38 (s, 9H). |
| 65 | 1.2 | 3,3- difluoro- pyrrolidine | 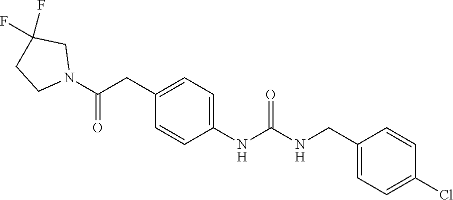 [SMILES: O=C(NCc1ccc(Cl)cc1)Nc1ccc(CC(=O)N2CCC(F)(F)C2)cc1] |
| | | | Compound 65. ({4-[2-(3,3-difluoropyrrolidinyl)-2- |
| | | | oxoethyl]phenyl}amino)-N-[(4- |
| | | | chlorophenyl)methyl]carboxamide. LCMS-ESI |
| | | | (POS.) m/z: 408.0 (M + H)+. 1H NMR (400 MHz, |
| | | | DMSO-d6) δ 8.62 (d, J = 1.2 Hz, 1H), 7.43-7.35 |
| | | | (m, 2H), 7.35-7.27 (m, 4H), 7.13-7.00 (m, 2H), |
| | | | 6.68 (t, J = 6.0 Hz, 1H), 4.28 (d, J = 6.0 Hz, 2H), |
| | | | 3.95 (t, J = 13.1 Hz, 1H), 3.77-3.64 (m, 2H), 3.57 |
| | | | (s, 1H), 3.55-3.47 (m, 2H), 2.48-2.41 (m, 1H), |
| | | | 2.37 (dq, J = 14.5, 7.1 Hz, 1H). |
| 107 | 1.2 | 2-ethoxy- cyclopropan- 1-amine (1:1 mixture diasatereo- mers) | 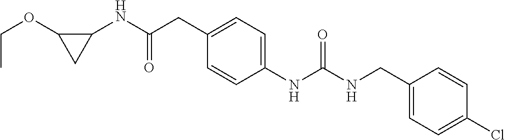 [SMILES: CCOC1CC1NC(=O)Cc1ccc(NC(=O)NCc2ccc(Cl)cc2)cc1] |
| | | | Compound 107. 2-[4-({N-[(4- |
| | | | chlorophenyl)methyl]carbamoyl}amino)phenyl]- |
| | | | N-(2-ethoxycyclopropyl)acetamide. LCMS-ESI |
| | | | (POS.) m/z: 402.0 (M + H)+. Diastereomer 1: 1H |
| | | | NMR (400 MHz, DMSO-d6) δ 8.57 (d, J = 1.5 Hz, |
| | | | 1H), 8.03-7.98 (m, 1H), 7.43-7.36 (m, 2H), 7.35- |
| | | | 7.28 (m, 4H), 7.13-7.03 (m, 2H), 6.64 (td, J = 6.0, |
| | | | 3.4 Hz, 1H), 4.28 (d, J = 5.9 Hz, 2H), 3.45-3.31 |
| | | | (m, 2H), 3.24 (s, 2H), 3.10 (ddd, J = 7.0, 3.7, 1.7 Hz, 1H), |
| | | | 2.55 (dtt, J = 7.2, 3.4, 1.8 Hz, 1H), 1.09 (t, J = 7.1 Hz, |
| | | | 3H), 0.94-0.85 (m, 1H), 0.68 (td, J = 6.7, 4.9 Hz, 1H). |
| | | | Diastereomer 2: 1H NMR (400 MHz, DMSO-d6) |
| | | | δ 8.57 (d, J = 1.5 Hz, 1H), 7.88-7.83 (m, 1H), 7.43- |
| | | | 7.36 (m, 2H), 7.35-7.28 (m, 4H), 7.13-7.03 (m, |
| | | | 2H), 6.64 (td, J = 6.0, 3.4 Hz, 1H), 4.28 (d, J = 5.9 |
| | | | Hz, 2H), 3.62-3.51 (m, 2H), 3.24 (s, 2H), 3.16 |
| | | | (ddd, J = 6.9, 5.2, 4.0 Hz, 1H), 2.65 (dq, J = 8.6, 5.3, |
| | | | 4.7 Hz, 1H), 1.02 (t, J = 7.0 Hz, 3H) 0.90-0.83 (m, |
| | | | 1H), 0.57 (ddd, J = 6.7, 5.5, 3.9 Hz, 1H). |
| 111 | 1.2 | tert-butyl (R)- pyrrolidin- 3- ylcarbamate | 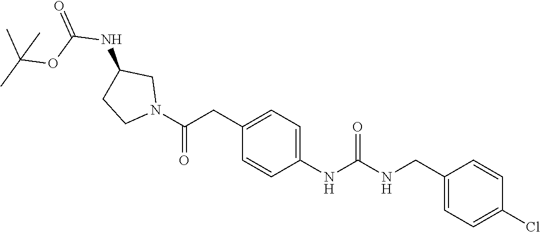 [SMILES: CC(C)(C)OC(=O)N[C@@H]1CCN(C(=O)Cc2ccc(NC(=O)NCc3ccc(Cl)cc3)cc2)C1] |
| | | | Compound 111. {[4-(2-{(3R)-3-[(tert- |
| | | | butoxy)carbonylamino]pyrrolidinyl}-2- |
| | | | oxoethyl)phenyl]amino}-N-[(4- |
| | | | chlorophenyl)methyl]carboxamide. LCMS-ESI |
| | | | (POS.) m/z: 387.0 (M-Boc + H)+. 1H NMR (400 |
| | | | MHz, DMSO-d6) δ 8.57 (d, J = 2.2 Hz, 1H), 7.43- |
| | | | 7.35 (m, 2H), 7.32 (dd, J = 8.4, 1.6 Hz, 4H), 7.20- |
| | | | 7.09 (m, 1H), 7.09-7.02 (m, 2H), 6.65 (td, J = 6.1, |
| | | | 1.9 Hz, 1H), 4.28 (d, J = 5.9 Hz, 2H), 3.98 (dd, J = |
| | | | 30.4, 6.6 Hz, 1H), 3.67-3.53 (m, 1H), 3.52-3.46 |
| | | | (m, 2H), 3.46-3.26 (m, 2H), 3.25-3.07 (m, 1H), |
| | | | 2.11-1.86 (m, 1H), 1.83-1.63 (m, 1H), 1.38 (d, J = |
| | | | 3.0 Hz, 9H). |
| 99 | 1.2 | 3-phenyl- cyclopentan- 1-amine (1:1 mixture of diastereo- mers) | 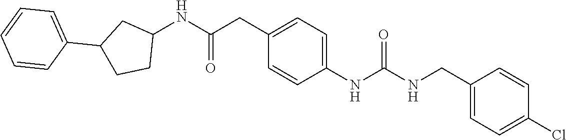 [SMILES: O=C(Cc1ccc(NC(=O)NCc2ccc(Cl)cc2)cc1)NC1CCC(c2ccccc2)C1] |
| | | | Compound 99. 2-[4-({[(4- |
| | | | chlorophenyl)methyl]amino}carbonylamino) |
| | | | phenyl]-N-(3-phenylcyclopentyl)acetamide. LCMS-ESI |
| | | | (POS.) m/z: 462.0 (M + H)+. Diastereomer 1: 1H |
| | | | NMR (400 MHz, DMSO-d6) δ 8.53 (s, 1H), 8.12 (t, |
| | | | J = 6.4 Hz, 1H), 7.44-7.34 (m, 2H), 7.34-7.20 (m, |
| | | | 8H), 7.20-7.07 (m, 3H), 6.62 (t, J = 6.0 Hz, 1H), |
| | | | 4.28 (d, J = 6.0 Hz, 2H), 4.17 (dq, J = 29.4, 7.1, 6.6 |
| | | | Hz, 1H), 3.30 (s, 2H), 3.01 (tt, J = 10.7, 7.1 Hz, 1H), |
| | | | 2.10 (h, J = 6.0 Hz, 1H), 2.01-1.88 (m, 1H), 1.88- |
| | | | 1.78 (m, 1H), 1.74-1.62 (m, 1H), 1.62-1.37 (m, 2H). |
| | | | Diastereomer 2: 1H NMR (400 MHz, DMSO-d6) δ |
| | | | 8.53 (s, 1H), 8.12 (t, J = 6.4 Hz, 1H), 7.44-7.34 (m, |
| | | | 2H), 7.34-7.20 (m, 8H), 7.20-7.07 (m, 3H), 6.62 |
| | | | (t, J = 6.0 Hz, 1H), 4.28 (d, J = 6.0 Hz, 2H), 4.21 (dq, |
| | | | J = 29.4, 7.1,6.6 Hz, 1H), 3.30(s, 2H), 3.21 (tt, J = |
| | | | 10.7, 7.1Hz, 1H), 2.30 (dt, J = 13.6, 7.1Hz, 1H), |
| | | | 2.01-1.88 (m, 1H), 1.88-1.78 (m, 1H), 1.74-1.62 |
| | | | (m, 1H), 1.62-1.37 (m, 2H). |
| 100 | 1.2 | 3- (methoxy- methyl) azetidine | 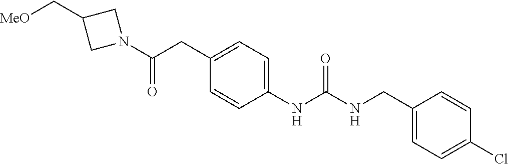 [SMILES: COCC1CN(C(=O)Cc2ccc(NC(=O)NCc3ccc(Cl)cc3)cc2)C1] |
| | | | Compound 100. {[(4- |
| | | | chlorophenyl)methyl]amino}-N-(4-{2-[3- |
| | | | (methoxy methyl)azetidinyl]-2- |
| | | | oxoethyl}phenyl)carboxamide. LCMS-ESI (POS.) |
| | | | m/z: 402.0 (M + H)+. 1H NMR (400 MHz, DMSO- |
| | | | d6) δ 8.54 (s, 1H), 7.42-7.35 (m, 2H), 7.35-7.26 |
| | | | (m, 4H), 7.11-7.01 (m, 2H), 6.62 (t, J = 6.0 Hz, |
| | | | 1H), 4.28 (d, J = 5.9 Hz, 2H), 4.16 (t, J = 8.4 Hz, |
| | | | 1H), 3.89-3.78 (m, 2H), 3.53 (dd, J = 9.5, 5.5 Hz, |
| | | | 1H), 3.44 (d, J = 6.4 Hz, 2H), 3.30 (s, 2H), 3.27 (s, |
| | | | 3H), 2.76 (s, 1H). |
| 93 | 1.2 | 3,3- difluoroaze- tidine | 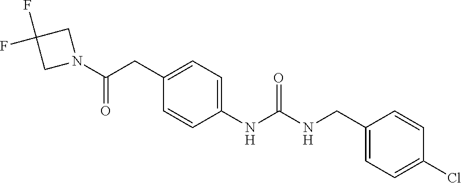 [SMILES: O=C(NCc1ccc(Cl)cc1)Nc1ccc(CC(=O)N2CC(F)(F)C2)cc1] |
| | | | Compound 93. N-{4-[2-(3,3-difluoroazetidinyl)-2-oxoethyl]phenyl} |
| | | | {[(4-chlorophenyl)methyl]amino}carboxamide. LCMS- |
| | | | ESI (POS.) m/z: 394.0 (M + H)+. 1H NMR (400 |
| | | | MHz, DMSO-d6) δ 8.57 (s, 1H), 7.53-7.35 (m, |
| | | | 2H), 7.35-7.27 (m, 4H), 7.15-7.02 (m, 2H), 6.64 |
| | | | (t, J = 6.0 Hz, 1H), 4.61 (t, J = 12.5 Hz, 2H), 4.36- |
| | | | 4.15 (m, 4H), 3.44 (s, 2H). |
| 132 | 1.2 | azetidine | 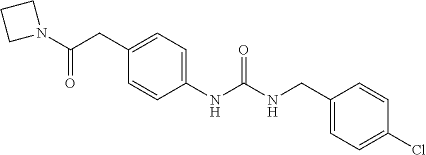 [SMILES: O=C(NCc1ccc(Cl)cc1)Nc1ccc(CC(=O)N2CCC2)cc1] |
| | | | Compound 132. N-[4-(2-azetidinyl-2-oxoethyl)phenyl]{[(4- |
| | | | chlorophenyl)methyl]amino}carboxamide. LCMS- |
| | | | ESI (POS.) m/z: 358.0 (M + H)+. 1H NMR (400 |
| | | | MHz, DMSO-d6) δ 8.54 (s, 1H), 7.45-7.35 (m, |
| | | | 2H), 7.35-7.25 (m, 4H), 7.12-7.01 (m, 2H), 6.62 |
| | | | (t, J = 6.0 Hz, 1H), 4.28 (d, J = 6.0 Hz, 2H), 4.18-4.07 (m, 2H), |
| | | | 3.82 (t, J = 7.7 Hz, 2H), 3.29 (s, 2H), 2.22-2.01 (m, 2H). |
| 124 | 1.2 | (S)- (tetrahydro- furan-3-yl) methanamine | 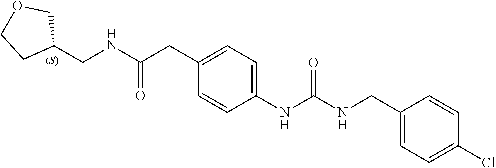 |
| | | | Compound 124. N-[((3S)oxolan-3-yl)methyl]-2-[4-({[(4- |
| | | | chlorophenyl)methyl]amino}carbonylamino) |
| | | | phenyl]acetamide. LCMS-ESI (POS.) m/z: 402.0 |
| | | | (M + H)+. 1H NMR (400 MHz, DMSO-d6) δ 8.55 (s, |
| | | | 1H), 8.07 (t, J = 5.8 Hz, 1H), 7.42-7.35 (m, 2H), |
| | | | 7.35-7.25 (m, 4H), 7.10 (d, J = 8.5 Hz, 2H), 6.64 (t, |
| | | | J = 6.0 Hz, 1H), 4.28 (d, J = 5.9 Hz, 2H), 3.76-3.53 |
| | | | (m, 4H), 3.30 (s, 2H), 3.11-2.95 (m, 2H), 2.36- |
| | | | 2.14 (m, 1H), 1.87 (dtd, J = 12.4, 8.0, 5.6 Hz, 1H), |
| | | | 1.49 (ddt, J = 12.4, 7.8, 6.3 Hz, 1H). |
| 130 | 1.2 | (S)-N,N- dimethyl- pyrrolidine- 3-amine | 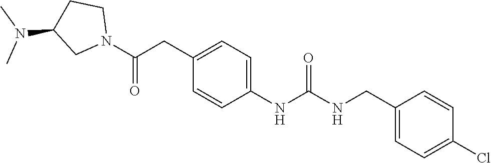 [SMILES: CN(C)[C@H]1CCN(C(=O)Cc2ccc(NC(=O)NCc3ccc(Cl)cc3)cc2)C1] |
| | | | Compound 130. N-(4-{2-[(3S)-3- |
| | | | (dimethylamino)pyrrolidinyl]-2-oxoethyl}phenyl){[(4- |
| | | | chlorophenyl)methyl]amino}carboxamide. LCMS- |
| | | | ESI (POS.) m/z: 415.1 (M + H)+. ¹H NMR (400 MHz, |
| | | | DMSO-d₆) δ 8.54 (s, 1H), 7.38 (s, 4H), 7.32 (d, J = |
| | | | 8.2 Hz, 4H), 7.22-6.33 (m, 3H), 4.28 (d, J = 5.9 Hz, |
| | | | 2H), 3.98-3.37 (m, 5H), 3.19 (td, J = 11.3, 10.6, 6.6 |
| | | | Hz, 1H), 2.29 (d, J = 35.1 Hz, 5H), 1.72 (d,J = 59.0 |
| | | | Hz, 1H), 1.22 (dt, J = 34.1, 6.6 Hz, 1H). |
| 75 | 1.2 | 2-(azetidin-3- yl)propan- 2-ol | 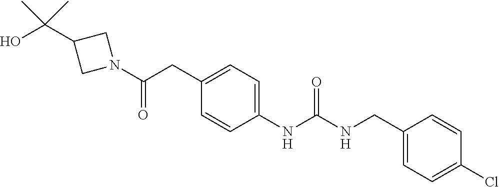 [SMILES: CC(C)(O)C1CN(C(=O)Cc2ccc(NC(=O)NCc3ccc(Cl)cc3)cc2)C1] |
| | | | Compound 75. {[(4-chlorophenyl)methyl]amino}- |
| | | | N-(4-{2-[3-(1-hydroxy-isopropyl)azetidinyl]-2- |
| | | | oxoethyl}phenyl)carboxamide. LCMS-ESI (POS.) |
| | | | m/z: 416.1 (M + H)+. ¹H NMR (400 MHz, DCM-d₂) δ |
| | | | 7.27 (m, 4H), 7.15 (d, J = 8.2 Hz, 2H), 7.02 (d, J = |
| | | | 8.2 Hz, 2H), 4.36 (s, 2H), 4.11 (s, 2H), 3.87 (s, 2H), |
| | | | 3.34 (s, 2H), 2.56 (p, J = 7.5 Hz, 1H), 1.13 (s, 6H). |
| 128 | 1.2 | (S)-N- methyl- pyrrolidine- 2- carboxamide | 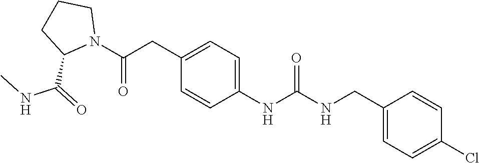 [SMILES: CNC(=O)[C@@H]1CCCN1C(=O)Cc1ccc(NC(=O)NCc2ccc(Cl)cc2)cc1] |
| | | | Compound 128. N-(4-{2-[(2S)-2-(N- |
| | | | methylcarbamoyl)pyrrolidinyl]-2-oxoethyl}phenyl){[(4- |
| | | | chlorophenyl)methyl]amino}carboxamide. LCMS- |
| | | | ESI (POS.) m/z: 429.2 (M + H)+. ¹H NMR (400 MHz, |
| | | | DMSO-d₆, 3:1 ratio of rotamers) δ 8.53 (s, 4H), 8.02 |
| | | | (d, J = 5.1 Hz, 1H), 7.73 (d, J = 5.0 Hz, 3H), 7.64- |
| | | | 7.22 (m, 23H), 7.13-7.06 (m, 6H), 6.97 (d, J = 8.1 |
| | | | Hz, 2H), 6.62 (t, J = 6.0 Hz, 3H), 4.54-4.13 (m, |
| | | | 11H), 3.78-3.35 (m, 13H), 3.27-2.95 (m, 1H), |
| | | | 2.72 (d, J = 5.6 Hz, 3H), 2.60 (d, J = 5.6 Hz, 9H), |
| | | | 2.20-2.05 (m, 2H), 2.03-1.73 (m, 17H) . |
| 38 | 1.2 | (S)- pyrrolidine- 2- carbonitrile | 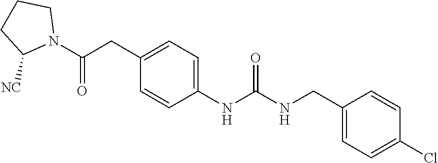 [SMILES: N#C[C@@H]1CCCN1C(=O)Cc1ccc(NC(=O)NCc2ccc(Cl)cc2)cc1] |
| | | | Compound 38. N-{4-[2-((2S)-2- |
| | | | cyanopyrrolidinyl)-2-oxoethyl]phenyl}{[(4- |
| | | | chlorophenyl)methyl]amino}carboxamide. LCMS- |
| | | | ESI (POS.) m/z: 397.2 (M + H)+. ¹H NMR (400 MHz, |
| | | | DMSO-d₆) δ 8.57 (s, 1H), 7.39 (d, J = 8.3 Hz, 2H), |
| | | | 7.37-7.28 (m, 4H), 7.09 (d, J = 8.2 Hz, 2H), 6.65 (t, |
| | | | J = 6.1 Hz, 1H), 4.74 (dd, J = 7.5, 3.6 Hz, 1H), 4.28 |
| | | | (d, J = 6.0 Hz, 2H), 3.62 (dd, J = 13.8, 5.8 Hz, 3H), |
| | | | 3.48 (q, J = 8.5 Hz, 1H), 2.14 (dd, J = 11.7, 5.2 Hz, |
| | | | 2H), 2.00 (q, J = 9.6, 8.2 Hz, 2H). |
| 117 | 1.2 | (S)- pyrrolidine- 2- carboxamide | 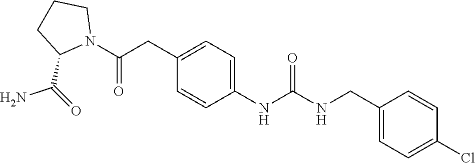 [SMILES: NC(=O)[C@@H]1CCCN1C(=O)Cc1ccc(NC(=O)NCc2ccc(Cl)cc2)cc1] |
| | | | Compound 117. (2S)-1-{2-[4-({[(4- |
| | | | chlorophenyl)methyl]amino}carbonylamino) |
| | | | phenyl]acetyl}pyrrolidine-2-carboxamide. LCMS-ESI |
| | | | (POS.) m/z: 415.00 (M + H)+. ¹H NMR (400 MHz, |
| | | | DMSO-d₆, 2:1 ratio of rotamers) δ 8.56 (s, 2H), 7.61 |
| | | | (s, 1H), 7.39 (d, J = 8.2 Hz, 3H), 7.32 (dd, J = 8.5, |
| | | | 2.1 Hz, 5H), 7.26 (s, 1H), 7.21 (s, 1H), 7.09 (d, J = |
| | | | 8.2 Hz, 2H), 7.03 (d, J = 8.2 Hz, 1H), 6.88 (s, 2H), |
| | | | 6.65 (t, J = 6.1 Hz, 2H), 4.35 (dd, J = 8.6, 2.8 Hz, 1H), |
| | | | 4.28 (d, J = 5.9 Hz, 3H), 4.20 (dd, J = 8.7, 2.9 Hz, 1H), |
| | | | 4.11 (q, J = 5.2 Hz, 1H), 3.69-3.53 (m, 4H), |
| | | | 3.51-3.41 (m, 2H), 3.18 (d, J = 5.1 Hz, 1H), 3.13 |
| | | | (d, J = 5.1 Hz, 1H), 2.23-2.09 (m, 1H), 2.08-1.66 (m, 7H). |
| 12 | 1.2 | 2- methyl- pyrrolidine | 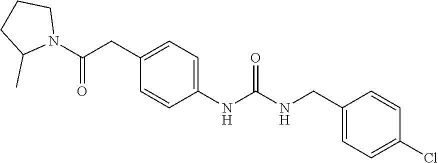 [SMILES: CC1CCCN1C(=O)Cc1ccc(NC(=O)NCc2ccc(Cl)cc2)cc1] |
| | | | Compound 12. {[(4-chlorophenyl)methyl]amino}- |
| | | | N-{4-[2-(2-methylpyrrolidinyl)-2- |
| | | | oxoethyl]phenyl}carboxamide. LCMS-ESI (POS.) |
| | | | m/z: 386.00 (M + H)+. ¹H NMR (400 MHz, DMSO-d₆) δ 8.54 |
| | | | (s, 1H), 7.39 (d, J = 8.3 Hz, 2H), 7.35- |
| | | | 7.24 (m, 4H), 7.08 (t, J = 8.5 Hz, 2H), 6.63 (t, J = 6.1 |
| | | | Hz, 1H), 4.28 (d, J = 5.9 Hz, 2H), 4.01 (td, J = 6.5, |
| | | | 2.5 Hz, 1H), 3.65-3.46 (m, 2H), 1.98-1.84 (m, 3H), |
| | | | 1.49 (dq, J = 8.1, 4.5, 3.6 Hz, 1H), 1.10 (d,6.4 |
| | | | Hz, 3H). |
| 13 | 1.2 | (S)-2- (methoxy- methyl) pyrrolidine | 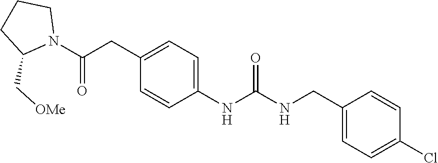 [SMILES: COC[C@@H]1CCCN1C(=O)Cc1ccc(NC(=O)NCc2ccc(Cl)cc2)cc1] |
| | | | Compound 13. N-(4-{2-[(2S)-2- |
| | | | (methoxymethyl)pyrrolidinyl]-2-oxoethyl}phenyl){[(4- |
| | | | chlorophenyl)methyl]amino}carboxamide. LCMS- |
| | | | ESI (POS.) m/z: 416.00 (M + H)+. ¹H NMR (400 |
| | | | MHz, DMSO-d₆) δ 8.55 (d, J = 2.9 Hz, 1H), 7.39 (d, |
| | | | J = 8.2 Hz, 2H), 7.32 (d, J = 8.3 Hz, 4H), 7.07 (d, J = |
| | | | 8.1 Hz, 2H), 6.64 (t, J = 6.1 Hz, 1H), 4.28 (d, J = 5.9 |
| | | | Hz, 2H), 4.14-4.03 (m, 1H), 3.60-3.47 (m, 2H), |
| | | | 3.47-3.37 (m, 4H), 3.23 (s, 3H), 1.99-1.68 (m, 4H). |
| 92 | 1.2 | azetidin-3- ylmethanol | 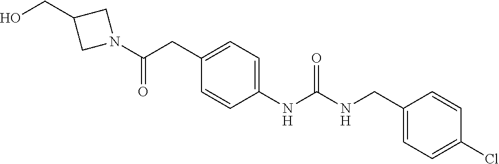 [SMILES: O=C(NCc1ccc(Cl)cc1)Nc1ccc(CC(=O)N2CC(CO)C2)cc1] |
| | | | Compound 92. {[(4-chlorophenyl)methyl]amino}- |
| | | | N-(4-{2-[3-(hydroxymethyl)azetidinyl]-2- |
| | | | oxoethyl}phenyl)carboxamide. LCMS-ESI (POS.) |
| | | | m/z: 388.10 (M + H)+. ¹H NMR (400 MHz, DMSO- |
| | | | d₆) δ 8.57 (s, 1H), 7.39 (d, J = 8.3 Hz, 2H), 7.32 |
| | | | (d, J = 8.4 Hz, 4H), 7.07 (d, J = 8.2 Hz, 2H), 6.66 |
| | | | (t, J = 6.0 Hz, 1H), 4.79 (t, J = 5.4 Hz, 1H), 4.28 |
| | | | (d, J = 5.9 Hz, 2H), 4.12 (t, J = 8.4 Hz, 1H), 3.89-3.77 |
| | | | (m, 2H), 3.59-3.46 (m, 3H), 3.30 (d, J = 5.2 Hz, 2H), |
| | | | 2.62 (tt, J = 8.4, 5.6 Hz, 1H). |
| 43 | 1.2 | thiomorpho- line 1,1- dioxide | 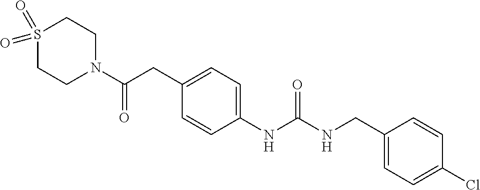 [SMILES: O=C(NCc1ccc(Cl)cc1)Nc1ccc(CC(=O)N2CCS(=O)(=O)CC2)cc1] |
| | | | Compound 43. N-{4-[2-(1,1-dioxo(1,4- |
| | | | thiazaperhydroin-4-yl))-2-oxoethyl]phenyl}{[(4- |
| | | | chlorophenyl)methyl]amino}carboxamide. LCMS- |
| | | | ESI (POS.) m/z: 436.00 (M + H)+. ¹H NMR (400 |
| | | | MHz, DMSO-d₆) δ 8.61 (s, 1H), 7.43-7.29 (m, 6H), |
| | | | 7.09 (d, J = 8.3 Hz, 2H), 6.69 (t, J = 6.0 Hz, 1H), |
| | | | 4.28 (d, J = 6.0 Hz, 2H), 3.87 (t, J = 5.3 Hz, 4H), |
| | | | 3.71 (s, 2H), 3.08 (q, J = 9.2, 7.2 Hz, 4H). |
| 57 | 1.2 | piperazin- 2-one | 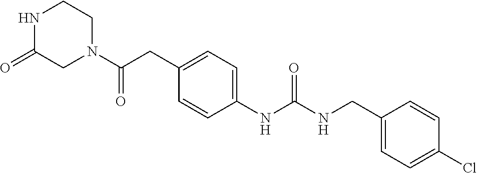 [SMILES: O=C1CN(C(=O)Cc2ccc(NC(=O)NCc3ccc(Cl)cc3)cc2)CCN1] |
| | | | Compound 57. {[(4-chlorophenyl)methyl]amino}-N-{4- |
| | | | [2-oxo-2-(3-oxopiperazinyl)ethyl]phenyl}carboxamide. |
| | | | LCMS-ESI (POS.) m/z: 401.00 (M + H)+. ¹H NMR |
| | | | (400 MHz, DMSO-d₆) δ 8.57 (d, J = 3.7 Hz, 1H), |
| | | | 8.01 (s, 1H), 7.39 (d, J = 8.2 Hz, 2H), 7.37-7.29 |
| | | | (m, 4H), 7.08 (t, J = 9.2 Hz, 2H), 6.65 (t, J = 6.0 Hz, |
| | | | 1H), 4.28 (d, J = 5.9 Hz, 2H), 4.04 (s, 1H), 3.94 (s, |
| | | | 1H), 3.63 (dd, J = 14.1, 8.9 Hz, 4H), 3.19-3.06 (m, 2H). |
| 34 | 1.2 | (R)-4,4- difluoro- pyrrolidine- 2- carboxamide | 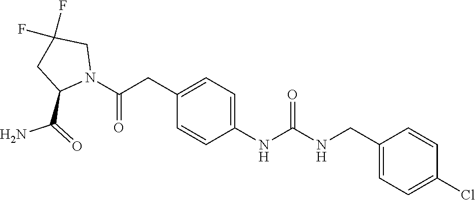 [SMILES: NC(=O)[C@H]1CC(F)(F)CN1C(=O)Cc1ccc(NC(=O)NCc2ccc(Cl)cc2)cc1] |
| | | | Compound 34. (2R)-1-{2-[4-({[(4- |
| | | | chlorophenyl)methyl]amino}carbonylamino) |
| | | | phenyl]acetyl}-4,4-difluoropyrrolidine-2-carboxamide. |
| | | | LCMS-ESI (POS.) m/z: 451.00 (M + H)+. ¹H NMR |
| | | | (400 MHz, DMSO-d_{6,} 5:2 ratio of rotamers) δ 8.55 |
| | | | (s, 1H), 7.45-7.36 (m, 3H), 7.36-7.29 (m, 4H), |
| | | | 7.14-7.01 (m, 3H), 6.64 (t, J = 6.1 Hz, 1H), 4.69 |
| | | | (dd, J = 9.6, 3.6 Hz, 1H), 4.45 (dd, J = 9.5, 4.5 Hz, |
| | | | 4H), 4.28 (d, J = 6.0 Hz, 2H), 4.10-3.98 (m, 2H), 3.73 |
| | | | (dd, J = 6.0, 4.5 Hz, 2H), 3.59 (s, 1H), 3.50 (d, J = 15.8 Hz, 1H), |
| | | | 2.99-2.81 (m, 1H), 2.79-2.67 (m, 1H), 2.43-2.27 (m, 1H). |
| 6 | 1.2 | (S)- pyrrolidin- 2-ylmethanol | 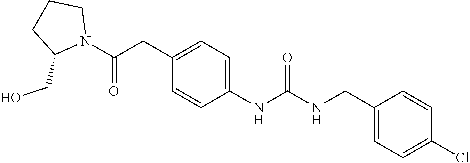 [SMILES: O=C(NCc1ccc(Cl)cc1)Nc1ccc(CC(=O)N2CCC[C@H]2CO)cc1] |
| | | | Compound 6. N-(4-{2-[(2S)-2- |
| | | | (hydroxymethyl)pyrrolidinyl]-2-oxoethyl}phenyl){[(4- |
| | | | chlorophenyl)methyl]amino}carboxamide. LCMS- |
| | | | ESI (POS.) m/z: 402.00 (M + H)+. ¹H NMR (400 |
| | | | MHz, DMSO-d₆) δ 8.59 (d, J = 4.3 Hz, 1H), 7.39 (d, |
| | | | J = 8.2 Hz, 2H), 7.32 (d, J = 8.2 Hz, 4H), 7.07 (dd, |
| | | | J = 8.6, 2.6 Hz, 2H), 6.69 (s, 1H), 4.75 (t, J = 5.7 Hz, |
| | | | 1H), 4.28 (d, J = 5.9 Hz, 1H), 4.01-3.90 (m, 2H), |
| | | | 3.52 (s, 2H), 3.52-3.38 (m, 3H), 3.33-3.18 (m, |
| | | | 2H), 1.90-1.74 (m, 2H). |
| 133 | 1.2 | (R)- N,N- dimethyl- pyrrolidin-3- amine | 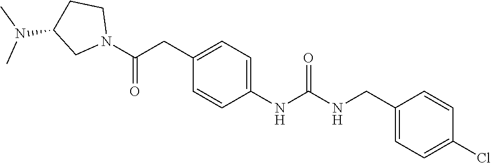 [SMILES: CN(C)[C@@H]1CCN(C(=O)Cc2ccc(NC(=O)NCc3ccc(Cl)cc3)cc2)C1] |
| | | | Compound 133. N-(4-{2-[(3R)-3- |
| | | | (dimethylamino)pyrrolidinyl]-2-oxoethyl}phenyl){[(4- |
| | | | chlorophenyl)methyl]amino}carboxamide. LCMS- |
| | | | ESI (POS.) m/z: 415.00 (M + H)+. ¹H NMR (400 |
| | | | MHz, DMSO-d₆) δ 8.53 (s, 1H), 7.39 (d, J = 8.3 Hz, |
| | | | 2H), 7.32 (d, J = 8.3 Hz, 4H), 7.08 (dd, J = 8.2, 5.7 |
| | | | Hz, 2H), 6.62 (t, J = 6.0 Hz, 1H), 4.28 (d, J = 6.0 Hz, |
| | | | 2H), 3.74 (d, J = 8.9 Hz, 1H), 3.63 (s, 1H), 3.52 (d, |
| | | | J = 7.8 Hz, 2H), 3.25-3.15 (m, 1H), 3.03 (s, 1H), |
| | | | 2.22 (s, 6H), 2.01 (s, 1H), 1.78 (s, 1H), 1.64 (s, 1H). |
| 51 | 1.2 | 1-(piperazin- 1-yl)ethan- 1-one | 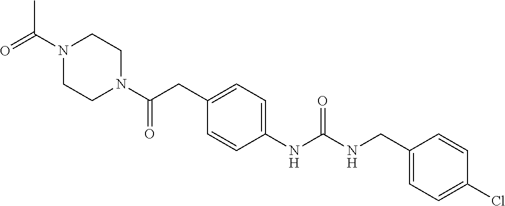 [SMILES: CC(=O)N1CCN(C(=O)Cc2ccc(NC(=O)NCc3ccc(Cl)cc3)cc2)CC1] |
| | | | Compound 51. N-{4-[2-(4-acetylpiperazinyl)-2-oxoethyl]phenyl} |
| | | | {[(4-chlorophenyl)methyl]amino}carboxamide. LCMS- |
| | | | ESI (POS.) m/z: 429.10 (M + H)+. ¹H NMR (400 |
| | | | MHz, DMSO-d₆) δ 8.55 (s, 1H), 7.39 (d, J = 8.3 Hz, |
| | | | 2H), 7.37-7.28 (m, 4H), 7.08 (d, J = 8.2 Hz, 2H), |
| | | | 6.63 (t, J = 6.1 Hz, 1H), 4.28 (d, J = 5.9 Hz, 2H), |
| | | | 3.65 (d, J = 4.7 Hz, 2H), 3.02 (h, J = 3.7 Hz, 8H), 2.00 (s, 3H) |
| 114 | 1.2 | oxetan- 3-amine | 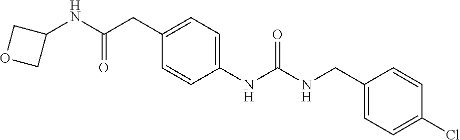 [SMILES: O=C(Cc1ccc(NC(=O)NCc2ccc(Cl)cc2)cc1)NC1COC1] |
| | | | Compound 114. 2-[4-({[(4- |
| | | | chlorophenyl)methyl]amino}carbonylamino)phenyl]- |
| | | | N-oxetan-3-ylacetamide. LCMS-ESI (POS.) |
| | | | m/z: 374.00 (M + H)+. ¹H NMR (400 MHz, DMSO- |
| | | | d₆) δ 8.75 (d,J = 6.4 Hz, 1H), 8.53 (s, 1H), 7.44- |
| | | | 7.27 (m, 5H), 7.10 (d, J = 8.3 Hz, 2H), 6.62 (t, J = |
| | | | 6.0 Hz, 1H), 4.76 (q, J = 6.7 Hz, 1H), 4.70 (t, J = 6.7 |
| | | | Hz, 2H), 4.40 (t, J = 5.9 Hz, 2H), 4.28 (d, J = 6.0 Hz, |
| | | | 2H), 3.25-3.30 (m, 2H). |
| 23 | 1.2 | tert- butyl (S)-2- methyl- piperazine- 1- carboxylate | 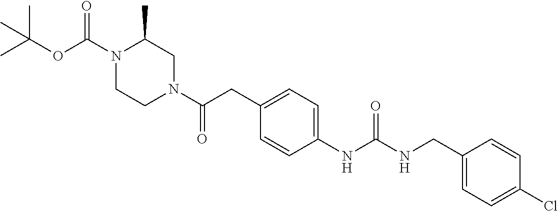 [SMILES: C[C@H]1CN(C(=O)Cc2ccc(NC(=O)NCc3ccc(Cl)cc3)cc2)CCN1C(=O)OC(C)(C)C] |
| | | | Compound 23. tert-butyl (2S)-4-{2-[4-({[(4- |
| | | | chlorophenyl)methyl]amino}carbonylamino)phenyl] |
| | | | acetyl}-2-methylpiperazinecarboxylate. LCMS- |
| | | | ESI (POS.) m/z: 445.00 (M-t-Bu + H)+. ¹H NMR |
| | | | (400 MHz, DMSO-d₆, 1:1 ratio of rotamers) δ 8.58 |
| | | | (d, J = 6.4 Hz, 2H), 7.45-7.26 (m, 12H), 7.15- |
| | | | 7.03 (m, 4H), 6.67 (d, J = 6.0 Hz, 2H), 4.28 (d, J = |
| | | | 5.9 Hz, 4H), 4.24-4.00 (m, 5H), 3.85 (d, J = 5.6 Hz, |
| | | | 1H), 3.81-3.49 (m, 8H), 3.2 (dd, J = 10.7, 5.6 Hz, |
| | | | 1H), 2.99 (ddd, J = 10.7, 5.6, 4.2 Hz, 1H), 2.89 (ddd, |
| | | | J = 5.6, 5.6, 4.2 Hz, 2H) 1.48-1.29 (m, 18H), 0.92 |
| | | | (dd, J = 7.0, 3.1 Hz, 6H). |
| 16 | 1.2 | tert-butyl (S)-2- (hydroxy- methyl) piperazine-1- carboxylate | 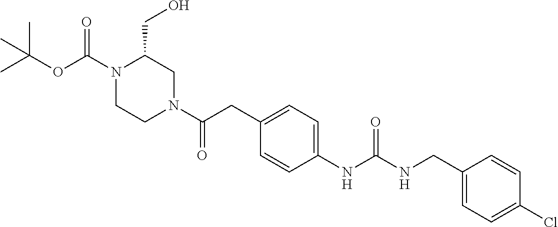 [SMILES: CC(C)(C)OC(=O)N1CCN(C(=O)Cc2ccc(NC(=O)NCc3ccc(Cl)cc3)cc2)C[C@H]1CO] |
| | | | Compound 16. tert-butyl (2S)-4-{2-[4-({[(4- |
| | | | chlorophenyl)methyl]amino}carbonylamino)phenyl]acetyl}-2- |
| | | | (hydroxymethyl)piperazinecarboxylate. LCMS- |
| | | | ESI (POS.) m/z: 461.00 (M-t-Bu + H)+. ¹H NMR |
| | | | (400 MHz, DMSO-d₆, 1:1 ratio of rotamers) δ 8.56 |
| | | | (d, J = 5.3 Hz, 2H), 7.45-7.24 (m, 12H), 7.07 (dd, |
| | | | J = 15.9, 8.2 Hz, 4H), 6.64 (q, J = 5.5 Hz, 2H), 5.02 (t, |
| | | | J = 5.2 Hz, 1H), 4.69 (t, J = 5.4 Hz, 1H), 4.28 (d, J = |
| | | | 5.9 Hz, 5H), 4.19 (d, J = 13.0 Hz, 1H), 4.03 (d, J = |
| | | | 13.7 Hz, 1H), 3.94 (s, 2H), 3.84 (d, J = 13.1 Hz, 1H), |
| | | | 3.78-3.50 (m, 6H), 3.41-3.30 (m, 3H), 3.26 (dd, |
| | | | J = 5.6, 3.9 Hz, 1H), 3.15 (dd, J = 13.8, 3.9 Hz, 1H), |
| | | | 3.05 (td, J = 5.6, 3.8 Hz, 1H), 2.90-2.75 (m, 2H), |
| | | | 2.67 (td, J = 12.4, 3.8 Hz, 2H), 1.40 (d, J = 6.6 Hz, 18H). |
| 11 | 1.2 | tert-butyl (R)-2- (hydroxy- methyl) piperazine-1- carboxylate | 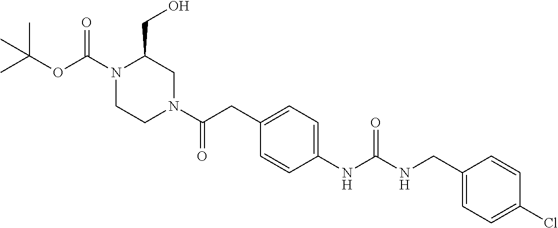 [SMILES: CC(C)(C)OC(=O)N1CCN(C(=O)Cc2ccc(NC(=O)NCc3ccc(Cl)cc3)cc2)C[C@@H]1CO] |
| | | | Compound 11. tert-butyl (2R)-4-{2-[4-({[(4-chlorophenyl) |
| | | | methyl]amino}carbonylamino)phenyl]acetyl}- |
| | | | 2-(hydroxymethyl)piperazinecarboxylate. LCMS- |
| | | | ESI (POS.) m/z: 461.00 (M-t-Bu + H)+. ¹H NMR |
| | | | (400 MHz, DMSO-d₆, 5:6 ratio of rotamers) δ 8.56 |
| | | | (d, J = 4.9 Hz, 2H), 7.52-7.23 (m, 12H), 7.07 (dd, |
| | | | J = 15.9, 8.2 Hz, 4H), 6.70-6.55 (m, 2H), 5.01 (t, J = |
| | | | 5.3 Hz, 1H), 4.68 (t, J = 5.4 Hz, 1H), 4.28 (d, J = 6.0 |
| | | | Hz, 5H), 4.19 (d, J = 12.9 Hz, 1H), 4.03 (d, J = 13.7 |
| | | | Hz, 1H), 3.94 (s, 2H), 3.84 (d, J = 13.1 Hz, 1H), 3.79- |
| | | | 3.51 (m, 6H), 3.40 (d, J = 6.8 Hz, 1H), 3.25 (d, J = |
| | | | 6.8 Hz, 2H), 3.15 (dd, J = 13.8, 3.9 Hz, 1H), 3.04 (t, |
| | | | J = 11.8 Hz, 1H), 2.87 (s, 1H), 2.79 (dd, J = 13.5, 4.1 |
| | | | Hz, 1H), 2.75-2.60 (m, 3H), 1.40 (d, J = 6.5 Hz, 18H). |
| 102 | 1.2 | tert-butyl 2,5- diazabicyclo [2.2.1] heptane-2- carboxylate | 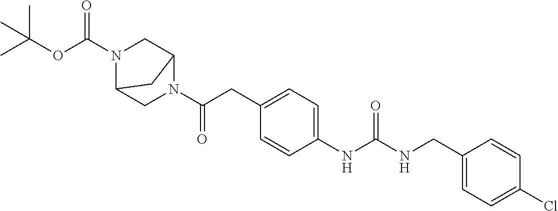 [SMILES: CC(C)(C)OC(=O)N1CC2CC1CN2C(=O)Cc1ccc(NC(=O)NCc2ccc(Cl)cc2)cc1] |
| | | | Compound 102. tert-butyl 5-{2-[4-({[(4- |
| | | | chlorophenyl)methyl]amino}carbonylamino)phenyl] |
| | | | acetyl}-2,5-diazabicyclo[2.2.1]heptane-2-carboxylate. |
| | | | LCMS-ESI (POS.) m/z: 443.00 (M-t-Bu + H)+. |
| | | | ¹H NMR (400 MHz, DMSO-d₆) δ 8.54 (d, |
| | | | J = 9.3 Hz, 1H), 7.43-7.28 (m, 6H), 7.08 (dd, J = |
| | | | 22.4, 6.9 Hz, 2H), 6.62 (q, J = 6.2 Hz, 1H), 4.68 (d, |
| | | | J = 16.7 Hz, 1H), 4.41-4.24 (m, 3H), 3.61 (s, 1H), |
| | | | 3.52 (d, J = 9.5 Hz, 1H), 3.41 (d, J = 16.6 Hz, 1H), |
| | | | 3.25 (dd, J = 29.0, 18.4 Hz, 1H), 3.19 (dd, J = 18.4, |
| | | | 10.5 Hz, 1H), 3.05 (dd, J = 29.0, 9.5 Hz, 1H), 1.75 |
| | | | (dd, J = 21.1, 11.2 Hz, 2H), 1.42-1.31 (m, 9H). |
| 4 | 1.2 | tert-butyl 2,5-diazabi- cyclo[4.1.0] heptane-2- carboxylate | 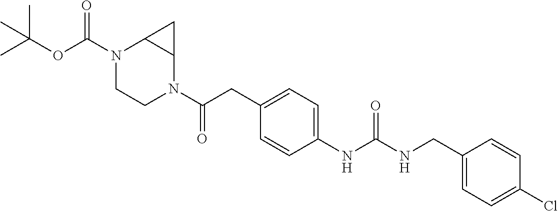 |
| | | | Compound 4. tert-butyl 5-{2-[4-({[(4- |
| | | | chlorophenyl)methyl]amino}carbonylamino)phenyl] |
| | | | acetyl}-2,5-diazabicyclo[4.1.0]heptane-2-carboxylate. |
| | | | LCMS-ESI (POS.) m/z: 443.00 (M-t-Bu + H)+. |
| | | | ¹H NMR (400 MHz, DMSO-d₆) δ 8.54 (s, |
| | | | 1H), 7.43-7.29 (m, 6H), 7.08 (dd, J = 15.2, 8.3 Hz, |
| | | | 2H), 6.62 (t, J = 6.0 Hz, 1H), 4.28 (d, J = 5.9 Hz, |
| | | | 2H), 3.80 (d, J = 15.5 Hz, 1H), 3.72-3.59 (m, 1H), |
| | | | 3.40 (d, J = 5.9 Hz, 2H), 3.08 (dd, J = 24.1, 12.6 Hz, |
| | | | 3H), 2.91 (s, 1H), 1.43 (s, 9H), 1.13 (q, J = 6.5 Hz, |
| | | | 1H), 0.60 (d, J = 5.8 Hz, 1H). |
| 22 | 1.2 | tert-butyl (R)-3- methyl- piperazine-1- carboxylate | 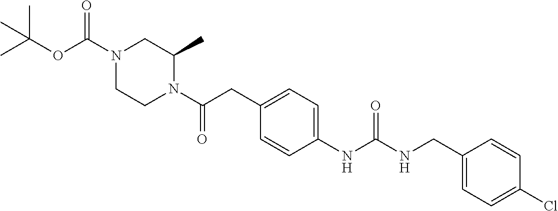 |
| | | | Compound 22. tert-butyl (3R)-4-{2-[4-({[(4- |
| | | | chlorophenyl)methyl]amino}carbonylamino)phenyl] |
| | | | acetyl}-3-methylpiperazinecarboxylate. LCMS- |
| | | | ESI (POS.) m/z: 445.05 (M-t-Bu + H)+. ¹H NMR (400 |
| | | | MHz, DMSO-d₆) δ 8.56 (s, 1H), 7.39 (d, J = 8.2 Hz, |
| | | | 2H), 7.34-7.26 (m, 4H), 7.07 (dd, J = 13.3, 8.0 Hz, |
| | | | 2H), 6.65 (t, J = 6.0 Hz, 1H), 4.28 (d, J = 5.9 Hz, |
| | | | 2H), 4.19 (d, J = 11.4 Hz, 1H), 4.03-3.42 (m, 6H), |
| | | | 2.91 (s, 1H), 2.73 (s, 2H), 1.40 (d, J = 4.9 Hz, 9H), |
| | | | 1.06 (d, J = 5.6 Hz, 3H). |
| 17 | 1.2 | tert-butyl (R)-3- (hydroxy- methyl) piperazine-1- carboxylate | 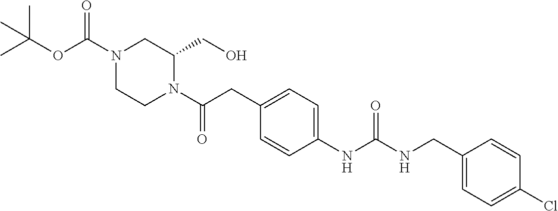 |
| | | | Compound 17. tert-butyl (3R)-4-{2-[4-({[(4-chlorophenyl) |
| | | | methyl]amino}carbonylamino)phenyl]acetyl}-3- |
| | | | (hydroxymethyl)piperazinecarboxylate. LCMS- |
| | | | ESI (POS.) m/z: 461.20 (M-t-Bu + H)+. ¹H NMR (400 |
| | | | MHz, DMSO-d₆) δ 8.54 (s, 1H), 7.39 (d, J = 8.3 Hz, |
| | | | 2H), 7.32 (d, J = 7.8 Hz, 4H), 7.06 (d, J = 8.3 Hz, |
| | | | 2H), 6.63 (t, J = 6.1 Hz, 1H), 4.95 (t, J = 5.3 Hz, |
| | | | 1H), 4.55 (t, J = 5.3 Hz, 1H), 4.37 (s, 1H), 4.28 (d, |
| | | | J = 6.0 Hz, 2H), 4.22 (d, J = 10.1 Hz, 1H), 3.98 (s, 1H), |
| | | | 3.87 (d, J = 13.1 Hz, 1H), 3.76 (d, J = 15.4 Hz, 1H), 3.61 |
| | | | (d, J = 12.4 Hz, 1H), 3.45 (s, 1H), 3.05 (t, J = 5.3 Hz, 1H), |
| | | | 2.84 (d, J = 8.6 Hz, 1H), 2.71 (s, 2H), 1.39 (s, 9H). |
| 27 | 1.2 | tert-butyl 3,3- dimethyl- piperazine-1- carboxylate | 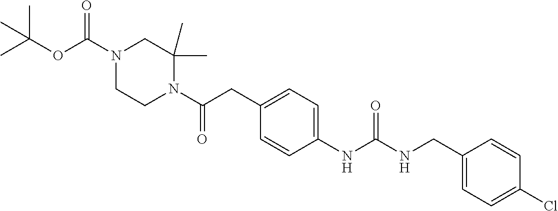 |
| | | | Compound 27. tert-butyl 4-{2-[4-({[(4- |
| | | | chlorophenyl)methyl]amino}carbonylamino) |
| | | | phenyl]acetyl}-3,3-dimethylpiperazinecarboxylate. |
| | | | LCMS-ESI (POS.) m/z: 459.00 (M-t-Bu + H)+. ¹H |
| | | | NMR (400 MHz, DMSO-d₆) δ 8.54 (s, 1H), 7.39 (d, |
| | | | J = 8.3 Hz, 2H), 7.36-7.26 (m, 4H), 7.05 (d, J = 8.2 |
| | | | Hz, 2H), 6.63 (t, J = 6.1 Hz, 1H), 4.28 (d, J = 5.9 Hz, |
| | | | 2H), 4.10 (q, J = 5.3 Hz, 4H), 3.55 (d, J = 15.5 Hz, |
| | | | 4H), 1.39 (d, J = 3.0 Hz, 9H), 1.35 (s, 6H). |
| 25 | 1.2 | tert-butyl 3,8-diazabi- cyclo[3.2.1] octane-3- carboxylate | 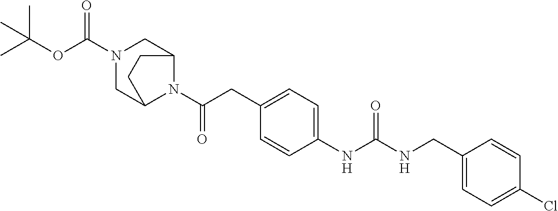 |
| | | | Compound 25. tert-butyl 8-{2-[4-({[(4- |
| | | | chlorophenyl)methyl]amino}carbonylamino) |
| | | | phenyl]acetyl}-3,8-diazabicyclo[3.2.1]octane-3- |
| | | | carboxylate. LCMS-ESI (POS.) m/z: 457.00 (M-t- |
| | | | Bu + H)+. ¹H NMR (400 MHz, DMSO-d₆) δ 8.55 (s, |
| | | | 1H), 7.39 (d, J = 8.3 Hz, 2H), 7.33 (q, J = 4.4 Hz, |
| | | | 4H), 7.11 (d, J = 8.2 Hz, 2H), 6.63 (t, J = 6.1 Hz, |
| | | | 1H), 4.48 (s, 1H), 4.39 (s, 1H), 4.28 (d, J = 5.9 Hz, |
| | | | 2H), 3.63-3.58 (m, 3H), 2.81-2.75 (m, 1H), 2.70-2.62 |
| | | | (m, 1H), 2.59-2.53 (m, 1H), 1.78-1.68 (m, 2H), |
| | | | 1.64-1.47 (m, 2H), 1.39 (s, 9H). |
| 52 | 1.2 | tert-butyl 3,8-diazabi- cyclo[3.2.1] octane-8- carboxylate | 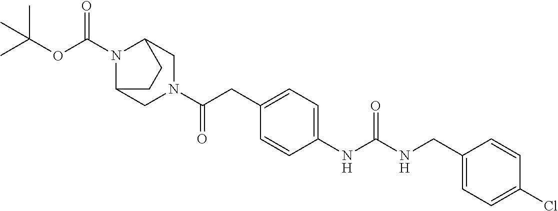 |
| | | | Compound 52. tert-butyl 3-{2-[4-({[(4- |
| | | | chlorophenyl)methyl]amino}carbonylamino)phenyl] |
| | | | acetyl}-3,8-diazabicyclo[3.2.1]octane-8-carboxylate. |
| | | | LCMS-ESI (POS.) m/z: 457.00 (M-t-Bu + H)+. |
| | | | ¹H NMR (400 MHz, DMSO-d₆) δ 8.55 (s, |
| | | | 1H), 7.39 (d, J = 8.2 Hz, 2H), 7.37-7.25 (m, 4H), |
| | | | 7.09 (d, J = 8.1 Hz, 2H), 6.63 (t, J = 6.1 Hz, 1H), |
| | | | 4.28 (d, J = 5.9 Hz, 2H), 4.09 (s, 1H), 4.03 (t, J = 6.5 |
| | | | Hz, 2H), 3.69 (d, J = 12.6 Hz, 1H), 3.65-3.48 (m, |
| | | | 2H), 3.12 (d, J = 12.5 Hz, 1H), 2.68 (d, J = 11.7 Hz, 1H), |
| | | | 1.76-1.57 (m, 3H), 1.47 (s, 9H), 1.25 (d, J = 9.3 Hz, 1H). |
| 32 | 1.2 | tert-butyl (S)-3-methyl- piperazine- 1- carboxylate | 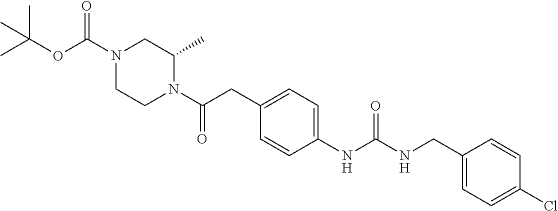 [SMILES: C[C@H]1CN(C(=O)OC(C)(C)C)CCN1C(=O)Cc1ccc(NC(=O)NCc2ccc(Cl)cc2)cc1] |
| | | | Compound 32. tert-butyl (3S)-4-{2-[4-({[(4- |
| | | | chlorophenyl)methyl]amino}carbonylamino)phenyl] |
| | | | acetyl}-3-methylpiperazinecarboxylate. LCMS- |
| | | | ESI (POS.) m/z: 445.00 (M-t-Bu + H)+. ¹H NMR (400 |
| | | | MHz, DMSO-d₆) δ 8.55 (s, 1H), 7.39 (d, J = 8.1 Hz, |
| | | | 2H), 7.35-7.23 (m, 4H), 7.06 (d, J = 10.1 Hz, 2H), |
| | | | 6.63 (t, J = 6.1 Hz, 1H), 4.55 (s, 1H), 4.28 (d, J = 6.0 |
| | | | Hz, 2H), 4.20 (s, 1H), 3.99-3.51 (m, 4H), 3.05 (dd, |
| | | | J = 22.9, 12.0 Hz, 1H), 2.91 (s, 1H), 2.71 (d, J = 22.4 |
| | | | Hz, 1H), 1.40 (s, 9H), 0.99 (d, J = 6.7 Hz, 3H). |
| 15 | 1.2 | tert-butyl (S)-3- (hydroxy- methyl) piperazine-1- carboxylate | 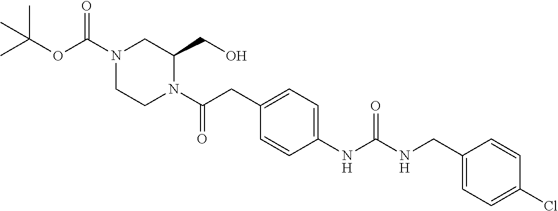 [SMILES: CC(C)(C)OC(=O)N1CCN(C(=O)Cc2ccc(NC(=O)NCc3ccc(Cl)cc3)cc2)[C@H](CO)C1] |
| | | | Compound 15. tert-butyl (3S)-4-{2-[4-({[(4-chlorophenyl) |
| | | | methyl]amino}carbonylamino)phenyl]acetyl}-3- |
| | | | (hydroxymethyl)piperazinecarboxylate. LCMS- |
| | | | ESI (POS.) m/z: 461.00 (M-t-Bu + H)+. ¹H NMR (400 |
| | | | MHz, DMSO-d₆) δ 8.54 (s, 1H), 7.39 (d, J = 8.2 Hz, |
| | | | 2H), 7.32 (d, J = 7.7 Hz, 4H), 7.16-6.91 (m, 2H), |
| | | | 6.62 (t, J = 6.0 Hz, 1H), 4.95 (s, 1H), 4.37 (s, 1H), |
| | | | 4.28 (d, J = 5.9 Hz, 2H), 4.20 (s, 1H), 3.99 (s, 1H), |
| | | | 3.87 (d, J = 13.1 Hz, 1H), 3.74 (t, J = 14.4 Hz, 1H), |
| | | | 3.65-3.55 (m, 1H), 3.45 (s, 1H), 3.07 (d, J = 12.8 |
| | | | Hz, 1H), 2.89 (d, J = 13.0 Hz, 1H), 2.69 (d, J = 12.1 |
| | | | Hz, 2H), 1.39 (s, 9H). |
| 21 | 1.2 | tert-butyl (3R,5S)- 3,5- dimethyl- piperazine-1- carboxylate | 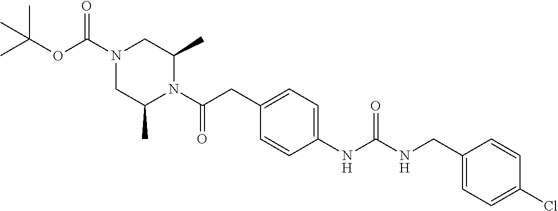 [SMILES: C[C@@H]1CN(C(=O)OC(C)(C)C)C[C@H](C)N1C(=O)Cc1ccc(NC(=O)NCc2ccc(Cl)cc2)cc1] |
| | | | Compound 21. tert-butyl (3S,5R)-4-{2-[4-({[(4- |
| | | | chlorophenyl)methyl]amino}carbonylamino) |
| | | | phenyl]acetyl}-3,5-dimethylpiperazinecarboxylate. |
| | | | LCMS-ESI (POS.) m/z: 459.10 (M-t-Bu + H)+. ¹H |
| | | | NMR (400 MHz, DMSO-d₆) δ 8.58 (s, 1H), 7.39 (d, |
| | | | J = 8.3 Hz, 2H), 7.33 (q, J = 3.9 Hz, 4H), 7.08 (d, J = |
| | | | 8.3 Hz, 2H), 6.67 (t, J = 6.0 Hz, 1H), 4.46 (s, 1H), |
| | | | 4.28 (d, J = 6.0 Hz, 2H), 4.13 (s, 1H), 3.78 (s, 2H), |
| | | | 3.70-3.49 (m, 2H), 2.90 (s, 2H), 1.41 (s, 9H), 1.08 |
| | | | (d, J = 6.8 Hz, 6H). |
| 69 | 1.2 | 1-methyl- azetidin-3- amine | 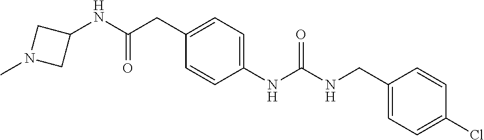 [SMILES: CN1CC(NC(=O)Cc2ccc(NC(=O)NCc3ccc(Cl)cc3)cc2)C1] |
| | | | Compound 69. 2-[4-({[(4- |
| | | | chlorophenyl)methyl]amino}carbonylamino) |
| | | | phenyl]-N-(1-methylazetidin-3-yl)acetamide. LCMS- |
| | | | ESI (POS.) m/z: 387.00 (M + H)+. ¹H NMR (400 |
| | | | MHz, DMSO-d₆) δ 8.52 (d, J = 7.8 Hz, 2H), 7.39 (d, |
| | | | J = 8.2 Hz, 2H), 7.32 (d, J = 8.1 Hz, 3H), 7.10 (d, J = |
| | | | 8.2 Hz, 2H), 6.62 (t, J = 6.1 Hz, 1H), 4.34 (q, J = 7.2 |
| | | | Hz, 1H), 4.28 (d, J = 6.0 Hz, 2H), 4.10 (q, J = 5.3 |
| | | | Hz, 1H), 3.88 (t, J = 8.5Hz, 2H), 3.38(s, 1H), 3.18 |
| | | | (d, J = 5.0 Hz, 2H), 2.54 (s, 3H). |
| 24 | 1.2 | (R)-1- benzyl-N- methyl- pyrrolidin-3- amine | 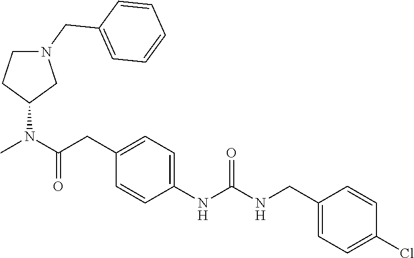 [SMILES: CN(C(=O)Cc1ccc(NC(=O)NCc2ccc(Cl)cc2)cc1)[C@@H]1CCN(Cc2ccccc2)C1] |
| | | | Compound 24. N-[(3R)-1-benzylpyrrolidin-3-yl]-2-[4-({[(4- |
| | | | chlorophenyl)methyl]amino}carbonylamino) |
| | | | phenyl]-N-methylacetamide. LCMS-ESI (POS.) m/z: |
| | | | 491.10 (M + H)+. ¹H NMR (400 MHz, DMSO-d₆) δ |
| | | | 9.68 (d, J = 59.9 Hz, 1H), 8.54 (d, J = 3.1 Hz, 1H), |
| | | | 7.45-7.39 (m, 5H), 7.33-7.25 (m, 5H), 7.05 (t, J = |
| | | | 8.1 Hz, 2H), 6.64 (t, J = 6.1 Hz, 1H), 5.11-4.79 (m, |
| | | | 1H), 4.67 (s, 1H), 4.42 (t, J = 5.1 Hz, 1H), 4.28 (d, |
| | | | J = 5.9 Hz, 2H), 3.83-3.50 (m, 3H), 3.16 (q, J = 10.1, |
| | | | 9.1 Hz, 2H), 2.96 (s, 3H), 2.34 (s, 1H), 2.20-1.75 (m, 2H). |
| 3 | 1.2 | tert-butyl (R)-2- methyl- piperazine-1- carboxylate | 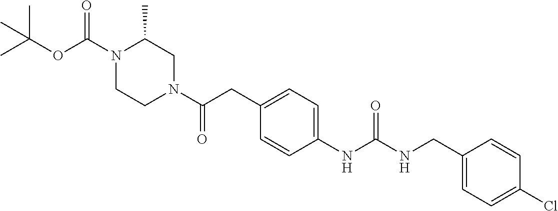 [SMILES: C[C@@H]1CN(C(=O)Cc2ccc(NC(=O)NCc3ccc(Cl)cc3)cc2)CCN1C(=O)OC(C)(C)C] |
| | | | Compound 3. tert-butyl (2R)-4-{2-[4-({[(4- |
| | | | chlorophenyl)methyl]amino}carbonylamino)phenyl] |
| | | | acetyl}-2-methylpiperazinecarboxylate. LCMS- |
| | | | ESI (POS.) m/z: 445.00 (M-t-Bu + H)+. ¹H NMR |
| | | | (400 MHz, DMSO-d_{6,} 1:1 ratio of rotamers) δ 8.58 |
| | | | (d, J = 6.4 Hz, 2H), 7.45-7.26 (m, 12H), 7.15- |
| | | | 7.03 (m, 4H), 6.67 (d, J = 6.0 Hz, 2H), 4.28 (d, J = |
| | | | 5.9 Hz, 4H), 4.24-4.00 (m, 5H), 3.85 (d, J = 5.6 Hz, |
| | | | 1H), 3.81-3.49 (m, 8H), 3.2 (dd, J = 10.7, 5.6 Hz, |
| | | | 1H), 2.99 (ddd, J = 10.7, 5.6, 4.2 Hz, 1H), 2.89 (ddd, |
| | | | J = 5.6, 5.6, 4.2 Hz, 2H) 1.48-1.29 (m, 18H), 0.92 |
| | | | (dd, J = 7.0, 3.1 Hz, 6H). |
| 139 | 1.2 | (R)-N- methyl- pyrrolidin-3- amine | 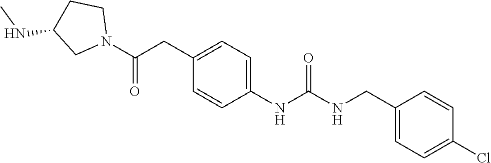 [SMILES: CN[C@@H]1CCN(C(=O)Cc2ccc(NC(=O)NCc3ccc(Cl)cc3)cc2)C1] |
| | | | Compound 139. [(4-{2-[(3R)-3- |
| | | | (methylamino)pyrrolidinyl]-2- |
| | | | oxoethyl}phenyl)amino]-N-[(4- |
| | | | chlorophenyl)methyl]carboxamide. LCMS-ESI |
| | | | (POS.) m/z: 401.20 (M + H)+. ¹H NMR (400 MHz, |
| | | | DMSO-d₆) δ 8.57 (s, 1H), 7.39 (d, J = 7.5 Hz, 2H), |
| | | | 7.32 (dd, J = 8.5, 1.5 Hz, 5H), 7.08 (d, J = 8.1 Hz, |
| | | | 2H), 6.67 (t, J = 6.0 Hz, 1H), 4.28 (d, J = 5.9 Hz, |
| | | | 2H), 3.66-3.42 (m, 3H), 3.29 (q, J = 5.8, 5.2 Hz, |
| | | | 0H), 3.22-2.93 (m, 2H), 2.24 (s, 3H), 1.92 (ddt, J = |
| | | | 27.6, 12.9, 6.8 Hz, 1H), 1.82-1.69 (m, 2H), 1.60 |
| | | | (dq, J = 12.7, 6.3 Hz, 1H). |
| 89 | 1.2 | (R)-N- methyl- pyrrolidin- 3-amine | 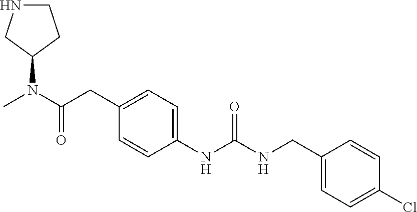 [SMILES: CN(C(=O)Cc1ccc(NC(=O)NCc2ccc(Cl)cc2)cc1)[C@@H]1CCNC1] |
| | | | Compound 89. N-((3R)pyrrolidin-3-yl)-2-[4-({N- |
| | | | [(4-chlorophenyl)methyl]carbamoyl}amino)phenyl]- |
| | | | N-methylacetamide. LCMS-ESI (POS.) m/z: 401.20 |
| | | | (M + H)+. ¹H NMR (400 MHz, DMSO-d₆, 1:1 ratio of |
| | | | rotamers) δ 8.60 (d, J = 4.2 Hz, 2H), 7.43-7.29 (m, |
| | | | 14H), 7.06 (dd, J = 8.6, 3.7 Hz, 4H), 6.69 (t, J = 6.1 |
| | | | Hz, 2H), 4.91 (p, J = 7.6 Hz, 1H), 4.46 (p, J = 7.4 |
| | | | Hz, 2H), 4.28 (d, J = 5.9 Hz, 4H), 3.67 (s, 2H), 3.57 |
| | | | (s, 2H), 2.90 (d, J = 5.5 Hz, 2H), 2.87 (s, 4H), 2.82 |
| | | | (dd, J = 11.6, 6.7 Hz, 1H), 2.79-2.53 (m, 6H), 1.94- |
| | | | 1.77 (m, 3H), 1.72 (dt, J = 14.2, 7.2 Hz, 2H), 1.55 |
| | | | (dp, J = 14.2, 7.3 Hz, 2H). |
| 72 | 1.2 | (1- methyl- piperazin-2- yl)methanol | 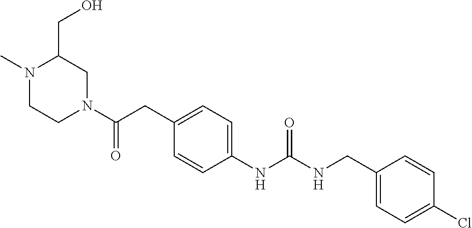 [SMILES: CN1CCN(C(=O)Cc2ccc(NC(=O)NCc3ccc(Cl)cc3)cc2)CC1CO] |
| | | | Compound 72. N-[(4-chlorophenyl)methyl][(4-{2- |
| | | | [3-(hydroxymethyl)-4-methylpiperazinyl]-2- |
| | | | oxoethyl}phenyl)amino]carboxamide. LCMS-ESI |
| | | | (POS.) m/z: 431.20 (M + H)+. ¹H NMR (400 MHz, |
| | | | DMSO-d₆, 1:1 ratio of rotamers) δ 8.47 (s, 2H), 7.32 |
| | | | (d, J = 7.2 Hz, 4H), 7.26-7.18 (m, 4H), 7.07 (d, J = |
| | | | 7.9 Hz, 4H), 6.95-6.77 (m, 4H), 6.50 (t, J = 6.0 Hz, |
| | | | 2H), 4.21 (d, J = 5.7 Hz, 4H), 4.18-4.11 (m, 2H), |
| | | | 4.09-3.94 (m, 1H), 3.87 (s, 1H), 3.73 (d, J = 1.5 |
| | | | Hz, 7H), 3.68 (d, J = 14.1 Hz, 1H), 3.62 (d,J = 6.6 |
| | | | Hz, 3H), 3.24 (d, J = 8.3 Hz, 1H), 3.14 (q, J = 12.2 |
| | | | Hz, 2H), 3.03 (s, 2H), 2.83 (dd, J = 24.3, 13.1 Hz, |
| | | | 3H), 2.51 (d, J = 1.8 Hz, 5H), 2.49-2.39 (m, 2H). |
| 285 | 1.1 | (1- methyl- piperazin-2- yl)methanol | 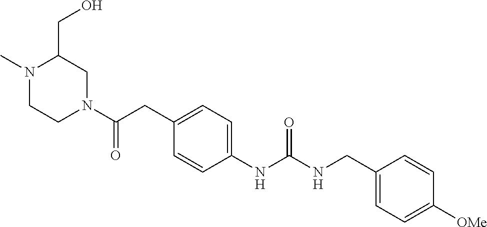 [SMILES: COc1ccc(CNC(=O)Nc2ccc(CC(=O)N3CCN(C)C(CO)C3)cc2)cc1] |
| | | | Compound 285. N-(4-{2-[3-(hydroxymethyl)-4- |
| | | | methylpiperazinyl]-2-oxoethyl}phenyl){[(4- |
| | | | methoxyphenyl)methyl]amino}carboxamide. |
| | | | LCMS-ESI (POS.) m/z: 427.20 (M + H)+. ¹H NMR |
| | | | (400 MHz, DMSO-d₆) δ 8.46 (s, 1H), 7.34 (d, J = 8.0 |
| | | | Hz, 2H), 7.23 (d, J = 8.1 Hz, 2H), 7.08 (d, J = 8.1 |
| | | | Hz, 2H), 6.90 (d, J = 8.1 Hz, 2H), 6.50 (t, J = 5.9 Hz, |
| | | | 1H), 5.41 (s, 1H), 4.22 (d, J = 5.7 Hz, 2H), 4.07 (s, |
| | | | 1H), 3.74 (s, 4H), 3.66 (dq, J = 17.4, 8.1, 7.1 Hz, |
| | | | 4H), 3.20 (s, 1H), 2.86 (s, 3H), 2.69 (s, 3H). |
| 104 | 1.2 | (4-methyl- piperazin-2- yl)methanol | 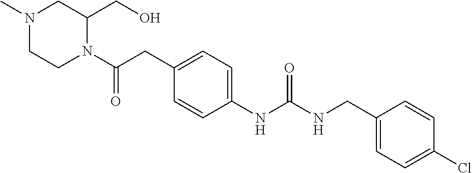 [SMILES: CN1CCN(C(=O)Cc2ccc(NC(=O)NCc3ccc(Cl)cc3)cc2)C(CO)C1] |
| | | | Compound 104. {[(4- |
| | | | chlorophenyl)methyl]amino}-N-(4-{2-[2- |
| | | | (hydroxymethyl)-4-methylpiperazinyl]-2- |
| | | | oxoethyl}phenyl)carboxamide. LCMS-ESI (POS.) |
| | | | m/z: 431.20 (M + H)+. ¹H NMR (400 MHz, DMSO- |
| | | | d₆) δ 10.16 (s, 1H), 9.01 (d, J = 8.0 Hz, 2H), 8.92 (d, |
| | | | J = 8.0 Hz, 2H), 8.76 (d, J = 8.1 Hz, 2H), 8.58 (d, J = |
| | | | 8.0 Hz, 2H), 8.20 (t, J = 5.9 Hz, 1H), 5.90 (d, J = 5.7 |
| | | | Hz, 2H), 5.42 (s, 2H), 5.29 (s, 2H), 4.20 (s, 4H), 4.06 |
| | | | (dt, J = 17.8, 4.9 Hz, 5H), 3.88 (d, J = 6.5 Hz, 2H). |
| 338 | 1.1 | (4-methyl- piperazin-2- yl)methanol | 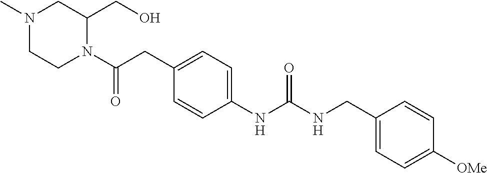 [SMILES: COc1ccc(CNC(=O)Nc2ccc(CC(=O)N3CCN(C)CC3CO)cc2)cc1] |
| | | | Compound 338. N-(4-{2-[2-(hydroxymethyl)-4- |
| | | | methylpiperazinyl]-2-oxoethyl}phenyl){[(4- |
| | | | methoxyphenyl)methyl]amino}carboxamide. |
| | | | LCMS-ESI (POS.) m/z: 427.20 (M + H)+. ¹H NMR |
| | | | (400 MHz, DMSO-d₆) δ 8.51 (s, 1H), 7.56 (s, 1H), |
| | | | 7.31 (d, J = 8.0 Hz, 2H), 7.22 (d, J = 7.9 Hz, 2H), |
| | | | 7.06 (d, J = 7.9 Hz, 2H), 6.93-6.86 (m, 2H), 6.51 (t, |
| | | | J = 5.9 Hz, 1H), 4.21 (d, J = 5.8 Hz, 2H), 3.73 (d, J = |
| | | | 1.5 Hz, 4H), 3.60 (s, 1H), 3.20 (s, 4H), 3.06 (dt, J = |
| | | | 17.8, 4.9 Hz, 5H), 2.88 (br s, J = 6.5 Hz, 4H). |
| 18 | 1.2 | 2-methyl-1- (piperazine- 1-yl) propan-2-ol | 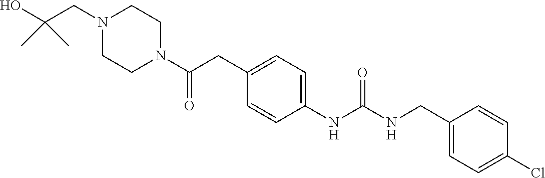 [SMILES: CC(C)(O)CN1CCN(C(=O)Cc2ccc(NC(=O)NCc3ccc(Cl)cc3)cc2)CC1] |
| | | | Compound 18. {[(4-chlorophenyl)methyl]amino}- |
| | | | N-(4-{2-[4-(2-hydroxy-2-methylpropyl)piperazinyl]-2- |
| | | | oxoethyl}phenyl)carboxamide. LCMS-ESI (POS.) |
| | | | m/z: 459.20 (M + H)+. ¹H NMR (400 MHz, DMSO- |
| | | | d₆) δ 8.46 (s, 1H), 7.32 (d, J = 8.0 Hz, 2H), 7.28- |
| | | | 7.18 (m, 2H), 7.07 (d, J = 8.0 Hz, 2H), 6.97-6.81 |
| | | | (m, 2H), 6.50 (t, J = 6.0 Hz, 1H), 4.22 (d, J = 5.6 Hz, |
| | | | 2H), 3.74 (s, 2H), 3.61 (s, 2H), 3.46 (s, 7H), 3.18 (q, |
| | | | J = 10.2 Hz, 2H), 2.51 (s, 6H). |
| 214 | 1.1 | 2-methyl-1- (piperazin-1- yl)propan-2- ol | 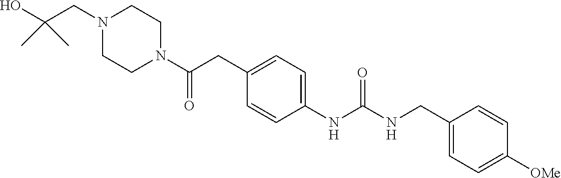 [SMILES: COc1ccc(CNC(=O)Nc2ccc(CC(=O)N3CCN(CC(C)(C)O)CC3)cc2)cc1] |
| | | | Compound 214. N-(4-{2-[4-(2-hydroxy-2- |
| | | | methylpropyl)piperazinyl]-2-oxoethyl}phenyl){[(4- |
| | | | methoxyphenyl)methyl]amino}carboxamide. |
| | | | LCMS-ESI (POS.) m/z: 455.20 (M + H)+. ¹H NMR |
| | | | (400 MHz, DMSO-d₆) δ 8.51 (d, J = 4.8 Hz, 1H), |
| | | | 7.78 (t, J = 7.7 Hz, 1H), 7.32 (d, J = 8.4 Hz, 2H), |
| | | | 7.23 (d, J = 8.1 Hz, 2H), 7.07 (d, J = 8.0 Hz, 2H), |
| | | | 6.90 (d, J = 8.0 Hz, 2H), 6.50 (t, J = 5.9 Hz, 1H), |
| | | | 4.22 (d, J = 5.7 Hz, 2H), 3.73 (s, 3H), 3.68 (s, 2H), |
| | | | 3.61 (s, 2H), 3.50 (s, 8H), 2.42 (d, J = 15.1 Hz, 4H), |
| | | | 1.26 (t, J = 5.4 Hz, 2H). |
| 220 | 1.1 | 2- (trifluoro- methyl) piperazine | 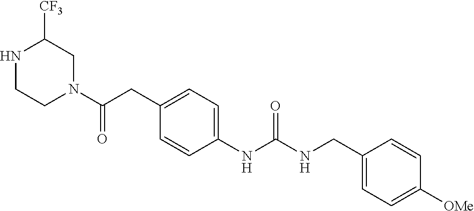 [SMILES: COc1ccc(CNC(=O)Nc2ccc(CC(=O)N3CCNC(C(F)(F)F)C3)cc2)cc1] |
| | | | Compound 220. N-[(4-methoxyphenyl)methyl][(4-{2-oxo-2-[3- |
| | | | (trifluoromethyl)piperazinyl]ethyl}phenyl)amino] |
| | | | carboxamide. LCMS-ESI (POS.) m/z: 451.20 |
| | | | (M + H)+. ¹H NMR (400 MHz, DMSO-d₆, 1:1 ratio of |
| | | | rotamers) δ 8.45 (s, 2H), 7.40-7.30 (m, 4H), 7.27- |
| | | | 7.20 (m, 4H), 7.07 (d, J = 7.8 Hz, 4H), 6.95-6.84 |
| | | | (m, 4H), 6.49 (d, J = 4.9 Hz, 2H), 4.27-4.12 (m, |
| | | | 6H), 4.02 (d, J = 12.7 Hz, 1H), 3.89 (d, J = 13.4 Hz, |
| | | | 1H), 3.86-3.57 (m, 12H), 3.57-3.39 (m, 1H), 3.25 |
| | | | (s, 1H), 3.12 (dt, J = 29.2, 14.2 Hz, 2H), 3.01-2.72 (m, 6H). |
| 229 | 1.1 | 1-cyclo- propyl- piperazine | 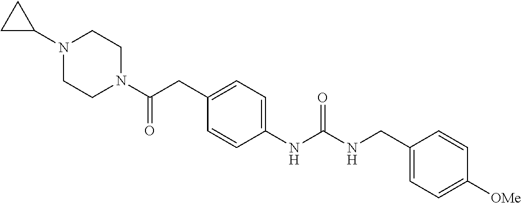 [SMILES: COc1ccc(CNC(=O)Nc2ccc(CC(=O)N3CCN(C4CC4)CC3)cc2)cc1] |
| | | | Compound 229. ({4-[2-(4-cyclopropylpiperazinyl)- |
| | | | 2-oxoethyl]phenyl}amino)-N-[(4- |
| | | | methoxyphenyl)methyl]carboxamide. LCMS-ESI |
| | | | (POS.) m/z: 423.20 (M + H)+. ¹H NMR (400 MHz, |
| | | | DMSO-d₆) δ 8.44 (s, 1H), 7.36-7.28 (m, 2H), 7.27- |
| | | | 7.19 (m, 2H), 7.07 (dd, J = 8.3, 2.8 Hz, 2H), 6.89 |
| | | | (dd, J = 8.7, 2.9 Hz, 2H), 6.53-6.44 (m, 1H), 4.21 |
| | | | (t, J = 4.3 Hz, 2H), 3.79-3.67 (m, 3H), 3.60 (d, J = |
| | | | 3.0 Hz, 2H), 3.40 (d, J = 5.2 Hz, 4H), 2.42 (dd, J = |
| | | | 19.2, 5.1 Hz, 4H), 1.58 (d, J = 6.2 Hz, 1H), 0.41 (t, |
| | | | J = 4.6 Hz, 2H), 0.31 (s, 2H). |
| 236 | 1.1 | 1-cyclo- propyl- methylpipe- razine | 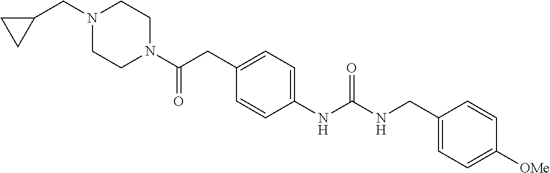 [SMILES: COc1ccc(CNC(=O)Nc2ccc(CC(=O)N3CCN(CC4CC4)CC3)cc2)cc1] |
| | | | Compound 236. [(4-{2-[4-(cyclopropylmethyl)piperazinyl]-2- |
| | | | oxoethyl}phenyl)amino]-N-[(4-methoxyphenyl)methyl]carboxamide. |
| | | | LCMS-ESI (POS.) m/z: 437.20 (M + H)+. ¹H NMR (400 MHz, |
| | | | DMSO-d₆) δ 8.40 (d, J = 3.3 Hz, 1H), 7.26 (t, J = 5.2 |
| | | | Hz, 2H), 7.22-7.13 (m, 2H), 7.02 (d, J = 7.4 Hz, |
| | | | 2H), 6.84 (dd, J = 8.4, 3.2 Hz, 2H), 6.44 (q, J = 5.2 |
| | | | Hz, 1H), 4.16 (t, J = 4.4 Hz, 2H), 3.68 (d, J = 3.3 Hz, |
| | | | 3H), 3.55 (d, J = 3.2 Hz, 2H), 3.41 (d, J = 5.3 Hz, |
| | | | 4H), 2.28 (dd, J = 17.6, 5.1 Hz, 4H), 2.11 (t, ./ = 4.8 |
| | | | Hz, 2H), 0.75 (s, 1H), 0.48-0.28 (m, 2H),-0.00 (t, J = 4.3 Hz, 2H). |
| 247 | 1.1 | 1-((1H- pyrazol-4- yl)methyl) piperazine | 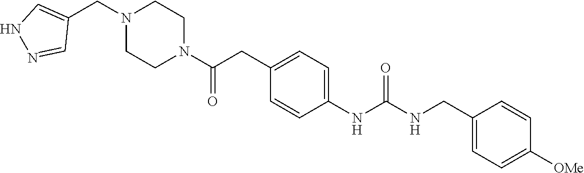 [SMILES: COc1ccc(CNC(=O)Nc2ccc(CC(=O)N3CCN(Cc4cn[nH]c4)CC3)cc2)cc1] |
| | | | Compound 247. N-[(4-methoxyphenyl)methyl][(4- |
| | | | {2-oxo-2-[4-(pyrazol-4-ylmethyl)piperazinyl]ethyl}phenyl)amino] |
| | | | carboxamide. LCMS-ESI (POS.) m/z: 463.20 (M + H)+. |
| | | | ¹H NMR (400 MHz, DMSO-d₆) δ 12.64 (s, 1H), 8.44 (s, |
| | | | 1H), 7.48 (s, 2H), 7.39-7.27 (m, 2H), 7.23 (dd, J = |
| | | | 8.6, 2.9 Hz, 2H), 7.05 (dd, J = 8.5, 3.0 Hz, 2H), 6.89 |
| | | | (dd, J = 8.7, 3.0 Hz, 2H), 6.49 (q, J = 5.2 Hz, 1H), |
| | | | 4.21 (s, 2H), 3.74 (t, J = 2.4 Hz, 3H), 3.59 (d, J = 3.2 |
| | | | Hz, 2H), 3.44 (s, 5H), 2.38-2.10 (m, 5H). |
| 228 | 1.1 | 1-(2,2,2- trifluoro- ethyl) piperazine | 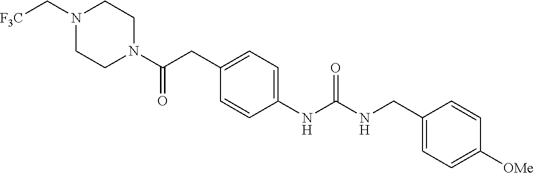 [SMILES: COc1ccc(CNC(=O)Nc2ccc(CC(=O)N3CCN(CC(F)(F)F)CC3)cc2)cc1] |
| | | | Compound 228. N-[(4-methoxyphenyl)methyl][(4-{2-oxo-2- |
| | | | [4-(2,2,2-trifluoroethyl)piperazinyl]ethyl}phenyl)amino] |
| | | | carboxamide. LCMS-ESI (POS.) m/z: 465.20 (M + H)+. |
| | | | ¹H NMR (400 MHz, DMSO-d₆) δ 8.44 (s, 1H), 7.36-7.28 (m, 2H), |
| | | | 7.23 (dd, J = 8.5, 2.9 Hz, 2H), 7.07 (dd, J = 8.3, 3.0 Hz, 2H), |
| | | | 6.94-6.86 (m, 2H), 6.49 (q, J = 5.2 Hz, 1H), 4.22 |
| | | | (s, 1H), 3.74 (t, J = 2.2 Hz, 3H), 3.61 (d, J = 3.2 Hz, 2H), |
| | | | 3.46 (s, 5H), 3.18 (tt, J = 10.4, 5.7 Hz, 2H), 2.50 (s, 5H). |
| 225 | 1.1 | 1-(pyridin- 2-ylmethyl) piperazine | 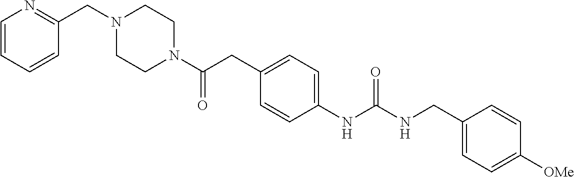 [SMILES: COc1ccc(CNC(=O)Nc2ccc(CC(=O)N3CCN(Cc4ccccn4)CC3)cc2)cc1] |
| | | | Compound 225. N-[(4-methoxyphenyl)methyl][(4-{2-oxo-2-[4-(2- |
| | | | pyridylmethyl)piperazinyl]ethyl}phenyl)amino] |
| | | | carboxamide. LCMS-ESI (POS.) m/z: 474.20 |
| | | | (M + H)+. ¹H NMR (400 MHz, DMSO-d₆) δ 8.47 (d, |
| | | | J = 21.3 Hz, 2H), 7.76 (d, J = 8.2 Hz, 1H), 7.43 (d, |
| | | | J = 7.8 Hz, 1H), 7.37-7.16 (m, 5H), 7.06 (dd, J = 8.6, |
| | | | 2.8 Hz, 2H), 6.90 (dd, J = 8.5, 2.9 Hz, 2H), 6.50 (d, |
| | | | J = 6.4 Hz, 1H), 4.22 (t, J = 4.1 Hz, 2H), 3.73 (d, J = |
| | | | 3.0 Hz, 3H), 3.66-3.58 (m, 4H), 3.48 (d, J = 5.6 Hz, |
| | | | 4H), 2.35 (d, J = 14.5 Hz, 4H). |
| 213 | 1.1 | 1-(oxetan-3- yl)piperazine | 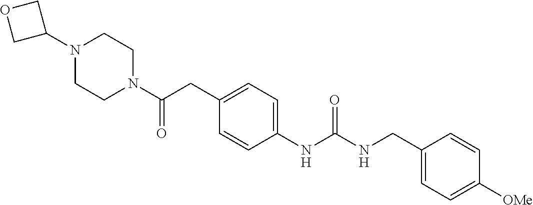 [SMILES: COc1ccc(CNC(=O)Nc2ccc(CC(=O)N3CCN(C4COC4)CC3)cc2)cc1] |
| | | | Compound 213. N-[(4-methoxyphenyl)methyl]({4- |
| | | | [2-(4-oxetan-3-ylpiperazinyl)-2- |
| | | | oxoethyl]phenyl}amino)carboxamide. LCMS-ESI |
| | | | (POS.) m/z: 439.20 (M + H)+. ¹H NMR (400 MHz, |
| | | | DMSO-d₆) δ 8.47 (d, J = 3.4 Hz, 1H), 7.73-7.29 |
| | | | (m, 2H), 7.25-7.16 (m, 2H), 7.07 (d, J = 8.0 Hz, |
| | | | 2H), 6.96-6.53 (m, 2H), 6.64-6.11 (m, 1H), 4.51 |
| | | | (d, J = 7.2 Hz, 2H), 4.42 (t, J = 4.9 Hz, 3H), 4.21 (t, |
| | | | J = 4.1 Hz, 2H), 3.74 (d, J = 3.1 Hz, 3H), 3.61 (d, J = |
| | | | 3.0 Hz, 2H), 3.48 (d, J = 5.3 Hz, 4H), 2.15 (dd, J = |
| | | | 19.1, 5.2 Hz, 4H). |
| 205 | 1.1 | 2- | 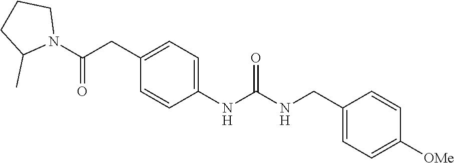 [SMILES: COc1ccc(CNC(=O)Nc2ccc(CC(=O)N3CCCC3C)cc2)cc1] |
| | | | Compound 205. {[(4-methoxyphenyl)methyl]amino}-N-{4-[2-(2- |
| | | | methylpyrrolidinyl)-2-oxoethyl]phenyl}carboxamide. |
| | | | LCMS-ESI (POS.) m/z: 382.10 (M + H)+. ¹H NMR (400 MHz, |
| | | | DMSO-d₆) δ 8.44 (s, 1H), 7.31 (d, J = 8.1 Hz, 2H), 7.23 (d, |
| | | | J = 8.2 Hz, 2H), 7.08 (t, J = 8.5 Hz, 2H), 6.90 (d, J = |
| | | | 8.2 Hz, 2H), 6.49 (t, J = 6.0 Hz, 1H), 4.22 (d, J = 5.8 |
| | | | Hz, 2H), 4.17-4.09 (m, 1H), 4.01 (t, J = 12 Hz, |
| | | | 1H), 3.73 (s, 3H), 3.56-3.40 (m, 3H), 1.88 (dtd, J = |
| | | | 23.9, 15.9, 7.7 Hz, 3H), 1.50 (d, J = 5.9 Hz, 1H), |
| | | | 1.10 (dd, J = 20.9, 6.4 Hz, 3H). |
| 206 | 1.1 | (S)-2- (pyrrolidin-2- yl)propan-2- ol | 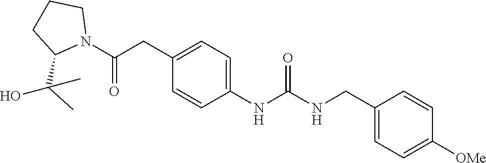 [SMILES: COc1ccc(CNC(=O)Nc2ccc(CC(=O)N3CCC[C@H]3C(C)(C)O)cc2)cc1] |
| | | | Compound 206. N-(4-{2-[(2S)-2-(1-hydroxy- |
| | | | isopropyl)pyrrolidinyl]-2-oxoethyl}phenyl){[(4- |
| | | | methoxyphenyl)methyl]amino}carboxamide. |
| | | | LCMS-ESI (POS.) m/z: 427.15 (M + H)+. ¹H NMR |
| | | | (400 MHz, DMSO-d₆) δ 8.47 (s, 1H), 7.33 (d, J = 8.1 |
| | | | Hz, 2H), 7.23 (d, J = 8.2 Hz, 2H), 7.09 (d, J = 8.2 |
| | | | Hz, 2H), 6.90 (d, J = 8.2 Hz, 2H), 6.51 (t, J = 6.0 Hz, |
| | | | 1H), 5.59 (s, 1H), 4.21 (d, J = 5.8 Hz, 2H), 3.98 (t, |
| | | | J = 6.5 Hz, 1H), 3.73 (s, 4H), 3.68-3.53 (m, 3H), |
| | | | 1.85 (d, J = 8.3 Hz, 2H), 1.77-1.57 (m, 2H), 1.01 |
| | | | (s, 3H), 0.95 (s, 3H). |
| 344 | 1.1 | (S)-2- pyrrolidine- 2- carboxamide | 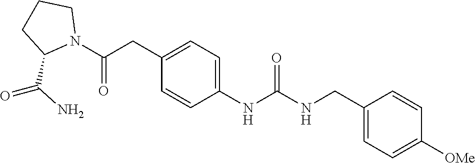 [SMILES: COc1ccc(CNC(=O)Nc2ccc(CC(=O)N3CCC[C@H]3C(N)=O)cc2)cc1] |
| | | | Compound 344. (2S)-1-{2-[4-({[(4- |
| | | | methoxyphenyl)methyl]amino}carbonylamino) |
| | | | phenyl]acetyl}pyrrolidine-2-carboxamide. LCMS- |
| | | | ESI (POS.) m/z: 411.10 (M + H)+. ¹H NMR (400 |
| | | | MHz, DMSO-d₆) δ 8.44 (s, 1H), 7.31 (d, J = 7.8 Hz, |
| | | | 2H), 7.27-7.19 (m, 3H), 7.06 (dd, J = 21.0, 8.2 Hz, |
| | | | 2H), 6.90 (d, J = 8.4 Hz, 3H), 6.48 (t, J = 5.9 Hz, |
| | | | 1H), 4.22 (d, J = 5.7 Hz, 3H), 3.74 (s, 3H), 3.67- |
| | | | 3.57 (m, 2H), 3.52-3.41 (m, 1H), 3.18 (d, J = 5.0 |
| | | | Hz, 1H), 1.88 (dddd, J = 47.8, 24.1, 14.6, 6.7 Hz, 4H). |
| 222 | 1.1 | (S)-2- pyrrolidin-2- ylmethanol | 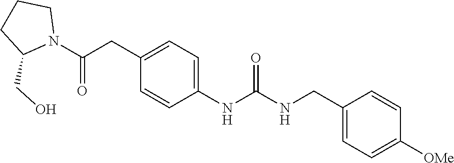 [SMILES: COc1ccc(CNC(=O)Nc2ccc(CC(=O)N3CCC[C@H]3CO)cc2)cc1] |
| | | | Compound 222. N-(4-{2-[(2S)-2- |
| | | | (hydroxymethyl)pyrrolidinyl]-2-oxoethyl}phenyl){[(4- |
| | | | methoxyphenyl)methyl]amino}carboxamide. |
| | | | LCMS-ESI (POS.) m/z: 398.10 (M + H)+. ¹H NMR |
| | | | (400 MHz, DMSO-d₆) δ 8.45 (d, J = 3.2 Hz, 1H), |
| | | | 7.31 (d, J = 7.8 Hz, 2H), 7.23 (d, J = 8.2 Hz, 2H), |
| | | | 7.07 (d, J = 8.5 Hz, 2H), 6.90 (d, J = 8.2 Hz, 2H), |
| | | | 6.52 (d, J = 6.3 Hz, 1H), 4.75 (t, J = 5.6 Hz, 1H), |
| | | | 4.21 (d, J = 5.8 Hz, 2H), 4.10 (d, J = 5.5 Hz, OH), |
| | | | 3.94 (s, 1H), 3.73 (s, 3H), 3.60-3.38 (m, 4H), 3.29- |
| | | | 3.13 (m, 2H), 1.83 (dd, J = 25.5, 9.5 Hz, 4H). |
| 209 | 1.1 | (S)-2- (methoxy- methyl) pyrrolidine | 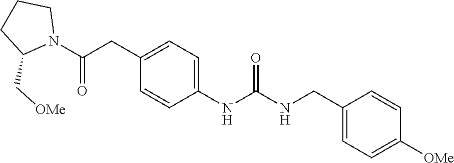 [SMILES: COC[C@@H]1CCCN1C(=O)Cc1ccc(NC(=O)NCc2ccc(OC)cc2)cc1] |
| | | | Compound 209. N-(4-{2-[(2S)-2- |
| | | | (methoxymethyl)pyrrolidinyl]-2-oxoethyl}phenyl){[(4- |
| | | | methoxyphenyl)methyl]amino}carboxamide. |
| | | | LCMS-ESI (POS.) m/z: 412.15 (M + H)+. ¹H NMR |
| | | | (400 MHz, DMSO-d₆) δ 8.43 (s, 1H), 7.36-7.26 (m, |
| | | | 2H), 7.23 (d, J = 8.2 Hz, 2H), 7.07 (d, J = 8.1 Hz, |
| | | | 2H), 6.90 (d, J = 8.3 Hz, 2H), 6.56-6.40 (m, 1H), |
| | | | 4.22 (d, J = 5.8 Hz, 2H), 4.09-4.01 (m, 1H), 3.73 |
| | | | (d, J = 1.2 Hz, 3H), 3.52 (d, J = 4.8 Hz, 1H), 3.42 |
| | | | (dt, J = 9.2, 4.3 Hz, 2H), 3.30 (d, J = 18.9 Hz, 3H), |
| | | | 3.23 (d, J = 1.2 Hz, 3H), 1.95-1.74 (m, 4H). |
| 336 | 1.1 | 1-methyl- azetidin-3- amine | 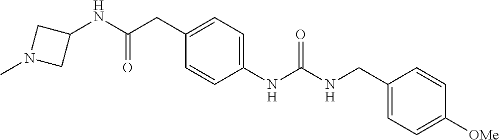 [SMILES: COc1ccc(CNC(=O)Nc2ccc(CC(=O)NC3CN(C)C3)cc2)cc1] |
| | | | Compound 336. 2-[4-({[(4- |
| | | | methoxyphenyl)methyl]amino}carbonylamino) |
| | | | phenyl]-N-(1-methylazetidin-3-yl)acetamide. LCMS- |
| | | | ESI (POS.) m/z: 383.10 (M + H)+. ¹H NMR (400 |
| | | | MHz, DMSO-d₆) δ 8.56-8.37 (m, 1H), 8.20 (s, 1H), |
| | | | 7.31 (d, J = 8.1 Hz, 2H), 7.22 (d, J = 8.2 Hz, 2H), |
| | | | 7.09 (d, J = 8.1 Hz, 2H), 6.89 (d, J = 8.3 Hz, 2H), |
| | | | 6.53 (t, J = 5.8 Hz, 1H), 4.21 (d, J = 5.9 Hz, 3H), |
| | | | 3.73 (s, 3H), 3.53 (t, J = 7.0 Hz, 3H), 2.98-2.83 (m, |
| | | | 2H), 2.26 (d, J = 1.8 Hz, 3H), 1.09 (d, J = 6.8 Hz, 1H). |
| 316 | 1.1 | azetidine- 3-ylmethanol | 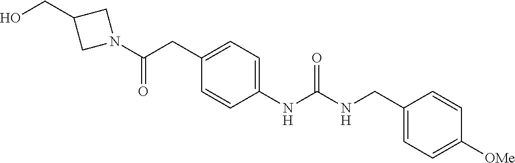 [SMILES: COc1ccc(CNC(=O)Nc2ccc(CC(=O)N3CC(CO)C3)cc2)cc1] |
| | | | Compound 316. N-(4-{2-[3- |
| | | | (hydroxymethyl)azetidinyl]-2-oxoethyl}phenyl) |
| | | | {[(4-methoxyphenyl)methyl]amino}carboxamide. |
| | | | LCMS-ESI (POS.) m/z: 384.10 (M + H)+. ¹H NMR |
| | | | (400 MHz, DMSO-d₆) δ 8.45 (s, 1H), 7.31 (d, J = 8.2 |
| | | | Hz, 2H), 7.23 (d, J = 8.2 Hz, 2H), 7.07 (d, J = 8.2 |
| | | | Hz, 2H), 6.90 (d, J = 8.2 Hz, 2H), 6.49 (t, J = 5.9 Hz, |
| | | | 1H), 4.79 (t, J = 5.3 Hz, 1H), 4.21 (d, J = 5.8 Hz, |
| | | | 2H), 4.13 (t, J = 8.4 Hz, 1H), 3.89-3.76 (m, 2H), 3.74 |
| | | | (s, 3H), 3.61-3.45 (m, 3H), 3.30 (s, 2H), 2.78-2.56 (m, 1H). |
| 253 | 1.1 | piperazin-2- one | 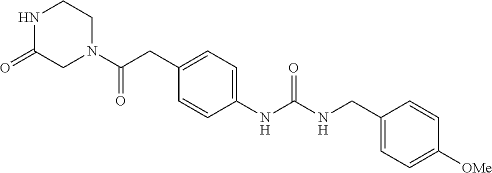 [SMILES: COc1ccc(CNC(=O)Nc2ccc(CC(=O)N3CCNC(=O)C3)cc2)cc1] |
| | | | Compound 253. {[(4- |
| | | | methoxyphenyl)methyl]amino}-N-{4-[2-oxo-2-(3- |
| | | | oxopiperazinyl)ethyl]phenyl}carboxamide. |
| | | | LCMS-ESI (POS.) m/z: 397.10 (M + H)+. ¹H NMR |
| | | | (400 MHz, DMSO-d₆) δ 8.45 (s, 1H), 8.05 (d, J = |
| | | | 34.4 Hz, 1H), 7.33 (d, J = 8.1 Hz, 2H), 7.23 (d, J = |
| | | | 8.1 Hz, 2H), 7.07 (t, J = 9.2 Hz, 2H), 6.90 (d, J = 8.2 |
| | | | Hz, 2H), 6.48 (t, J = 5.8 Hz, 1H), 4.21 (d, J = 5.8 Hz, |
| | | | 2H), 4.04 (s, 1H), 3.94 (s, 1H), 3.73 (d, J = 1.2 Hz, |
| | | | 3H), 3.63 (dd, J = 14.3, 8.5 Hz, 3H), 3.30 (s, 1H), |
| | | | 3.13 (d, J = 19.9 Hz, 2H). |
| 279 | 1.1 | (R)- pyrrolidine- 2-carbonitrile | 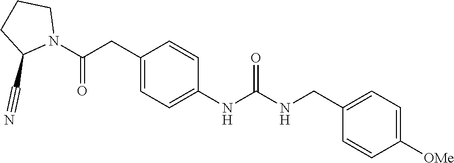 [SMILES: COc1ccc(CNC(=O)Nc2ccc(CC(=O)N3CCC[C@@H]3C#N)cc2)cc1] |
| | | | Compound 279. N-{4-[2-((2R)-2- |
| | | | cyanopyrrolidinyl)-2-oxoethyl]phenyl}{[(4- |
| | | | methoxyphenyl)methyl]amino}carboxamide. |
| | | | LCMS-ESI (POS.) m/z: 393.10 (M + H)+. ¹H NMR |
| | | | (400 MHz, DMSO-d₆) δ 8.47 (s, 1H), 7.34 (d, J = 8.0 |
| | | | Hz, 2H), 7.23 (d, J = 8.1 Hz, 2H), 7.09 (d, J = 8.1 |
| | | | Hz, 2H), 6.90 (d, J = 8.1 Hz, 2H), 6.50 (t, J = 5.9 Hz, |
| | | | 1H), 4.74 (dd, J = 7.6, 3.6 Hz, 1H), 4.22 (d, J = 5.8 |
| | | | Hz, 2H), 3.73 (d, J = 1.2 Hz, 3H), 3.67-3.58 (m, |
| | | | 2H), 3.53-3.42 (m, 1H), 2.22-1.90 (m, 4H). |
| 237 | 1.1 | (S)- pyrrolidine- 2-carbonitrile | 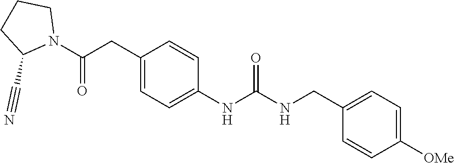 [SMILES: COc1ccc(CNC(=O)Nc2ccc(CC(=O)N3CCC[C@H]3C#N)cc2)cc1] |
| | | | Compound 237. N-{4-[2-((2S)-2- |
| | | | cyanopyrrolidinyl)-2-oxoethyl]phenyl}{[(4- |
| | | | methoxyphenyl)methyl]amino}carboxamide. |
| | | | LCMS-ESI (POS.) m/z: 393.10 (M + H)+. ¹H NMR |
| | | | (400 MHz, DMSO-d₆) δ 8.47 (s, 1H), 7.34 (d, J = 8.0 |
| | | | Hz, 2H), 7.23 (d, J = 8.1 Hz, 2H), 7.09 (d, J = 8.1 |
| | | | Hz, 2H), 6.90 (d, J = 8.1 Hz, 2H), 6.50 (t, J = 5.9 Hz, |
| | | | 1H), 4.74 (dd, J = 7.6, 3.6 Hz, 1H), 4.22 (d, J = 5.8 |
| | | | Hz, 2H), 3.73 (d, J = 1.2 Hz, 3H), 3.67-3.58 (m, |
| | | | 2H), 3.53-3.42 (m, 1H), 2.22-1.90 (m, 4H). |
| 159 | 1.3 | (R)- pyrrolidin- 2-ylmethanol | 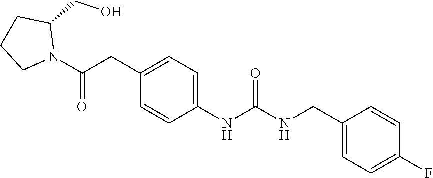 [SMILES: O=C(NCc1ccc(F)cc1)Nc1ccc(CC(=O)N2CCC[C@@H]2CO)cc1] |
| | | | Compound 159. N-(4-{2-[(2R)-2- |
| | | | (hydroxymethyl)pyrrolidinyl]-2-oxoethyl}phenyl) |
| | | | {[(4-fluorophenyl)methyl]amino}carboxamide. |
| | | | LCMS-ESI (POS.) m/z: 386.05 (M + H)+. ¹H NMR |
| | | | (400 MHz, DMSO-d₆) δ 8.52 (d, J = 3.2 Hz, 1H), 7.42- |
| | | | 7.28 (m, 4H), 7.16 (t, J = 8.7 Hz, 2H), 7.08 (dd, J = |
| | | | 8.5, 2.8 Hz, 2H), 6.62 (t, J = 6.0 Hz, 1H), 4.75 (t, J = |
| | | | 5.8 Hz, 1H), 4.28 (d, J = 5.9 Hz, 2H), 4.10 (d, J = 5.7 |
| | | | Hz, 1H), 4.04-3.85 (m, 1H), 3.52 (s, 2H), 3.44 (t, |
| | | | J = 4.5 Hz, 1H), 3.30-3.22 (m, 1H), 3.18 (d, J = 3.9 |
| | | | Hz, 2H), 1.84 (dtd, J = 23.5, 12.4, 10.9, 6.5 Hz, 5H). |
| 168 | 1.3 | (S)- pyrrolidin-3- ylmethanol | 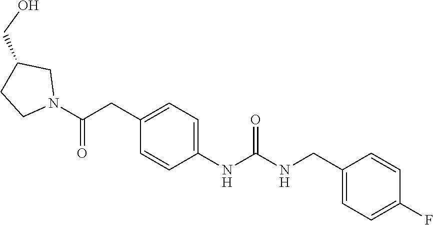 [SMILES: O=C(NCc1ccc(F)cc1)Nc1ccc(CC(=O)N2CC[C@H](CO)C2)cc1] |
| | | | Compound 168. N-(4-{2-[(3S)-3- |
| | | | (hydroxymethyl)pyrrolidinyl]-2-oxoethyl}phenyl){[(4- |
| | | | fluorophenyl)methyl]amino}carboxamide. LCMS- |
| | | | ESI (POS.) m/z: 386.05 (M + H)+. ¹H NMR (400 |
| | | | MHz, DMSO-d₆) δ 8.51 (s, 1H), 7.38-7.29 (m, 4H), |
| | | | 7.16 (t, J = 8.7 Hz, 2H), 7.07 (d, J = 8.1 Hz, 2H), |
| | | | 6.61 (t, J = 6.0 Hz, 1H), 4.68 (dt, J = 13.9, 5.3 Hz, |
| | | | 1H), 4.27 (d, J = 5.9 Hz, 2H), 3.61-3.48 (m, 3H), |
| | | | 3.45-3.36 (m, 2H), 3.28-3.16 (m, 1H), 3.10-3.00 |
| | | | (m, 1H), 2.39-2.16 (m, 2H), 1.98-1.78 (m, 1H), |
| | | | 1.72-1.46 (m, 1H). |
| 164 | 1.3 | (S)- pyrrolidin-2- ylmethanol | 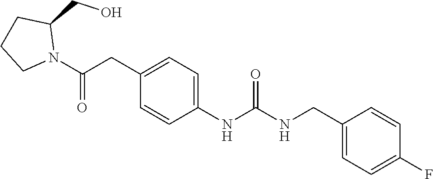 [SMILES: O=C(NCc1ccc(F)cc1)Nc1ccc(CC(=O)N2CCC[C@H]2CO)cc1] |
| | | | Compound 164. N-(4-{2-[(2S)-2- |
| | | | (hydroxymethyl)pyrrolidinyl]-2-oxoethyl}phenyl){[(4- |
| | | | fluorophenyl)methyl]amino}carboxamide. LCMS- |
| | | | ESI (POS.) m/z: 386.05 (M + H)+. ¹H NMR (400 |
| | | | MHz, DMSO-d₆) δ 8.51 (d, J = 3.1 Hz, 1H), 7.40- |
| | | | 7.27 (m, 4H), 7.16 (t, J = 8.7 Hz, 2H), 7.08 (dd, J = |
| | | | 8.6, 2.7 Hz, 2H), 6.61 (t, J = 6.0 Hz, 1H), 4.75 (t, J = |
| | | | 5.7 Hz, 1H), 4.28 (d, J = 5.9 Hz, 2H), 4.05-3.83 (m, |
| | | | 1H), 3.52 (s, 2H), 3.43 (dd, J = 7.8, 4.6 Hz, 1H), |
| | | | 3.29-3.20 (m, 1H), 1.97-1.69 (m, 4H). |
| 175 | 1.3 | (R)- pyrrolidin-3- ylmethanol | 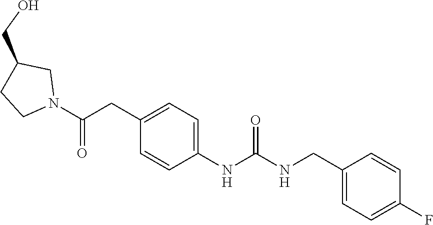 [SMILES: O=C(NCc1ccc(F)cc1)Nc1ccc(CC(=O)N2CC[C@@H](CO)C2)cc1] |
| | | | Compound 175. N-(4-{2-[(3R)-3- |
| | | | (hydroxymethyl)pyrrolidinyl]-2-oxoethyl}phenyl){[(4- |
| | | | fluorophenyl)methyl]amino}carboxamide. LCMS- |
| | | | ESI (POS.) m/z: 386.05 (M + H)+. ¹H NMR (400 |
| | | | MHz, DMSO-d₆) δ 8.51 (s, 1H), 7.38-7.26 (m, 4H), |
| | | | 7.16 (t, J = 8.7 Hz, 2H), 7.07 (d, J = 8.1 Hz, 2H), |
| | | | 6.60 (t, J = 6.1 Hz, 1H), 4.68 (dt, J = 13.6, 5.2 Hz, |
| | | | 1H), 4.27 (d, J = 5.9 Hz, 2H), 3.63-3.47 (m, 3H), |
| | | | 3.40 (dt, J = 11.8, 6.3 Hz, 2H), 3.28-3.13 (m, 1H), |
| | | | 3.09-2.99 (m, 1H), 2.39-2.13 (m, 1H), 2.03-1.74 |
| | | | (m, 1H), 1.70-1.49 (m, 1H). |
| 150 | 1.3 | tert-butyl (S)-2- (hydroxy- methyl) piperazine-1- carboxylate | 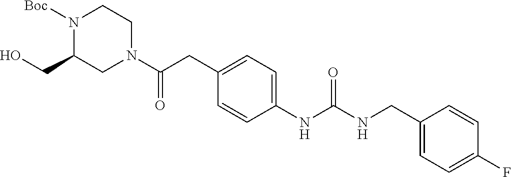 [SMILES: CC(C)(C)OC(=O)N1CCN(C(=O)Cc2ccc(NC(=O)NCc3ccc(F)cc3)cc2)C[C@H]1CO] |
| | | | Compound 150. tert-butyl (2S)-4-{2-[4-({[(4-fluorophenyl) |
| | | | methyl]amino}carbonylamino)phenyl]acetyl}-2- |
| | | | (hydroxymethyl)piperazinecarboxylate. LCMS- |
| | | | ESI (POS.) m/z: 501.20 (M + H)+. ¹H NMR (400 |
| | | | MHz, DMSO-d₆) δ 8.52 (d, J = 4.3 Hz, 1H), 7.39- |
| | | | 7.27 (m, 4H), 7.16 (t, J = 8.6 Hz, 2H), 7.07 (dd, J = |
| | | | 15.8, 8.1 Hz, 2H), 6.65-6.54 (m, 1H), 5.01 (t, J = |
| | | | 5.3 Hz, 1H), 4.68 (t, J = 5.5 Hz, 1H), 4.33 (d, J = |
| | | | 14.5 Hz, 1H), 4.28 (d, J = 5.9 Hz, 2H), 4.20 (d, J = |
| | | | 12.9 Hz, 1H), 4.09-3.79 (m, 2H), 3.77-3.50 (m, |
| | | | 3H), 3.30-2.97 (m, 2H), 2.94-2.62 (m, 2H), 1.40 (s, 9H). |
| 153 | 1.3 | tert-butyl (R)-2- (hydroxy- methyl) piperazine-1- carboxylate | 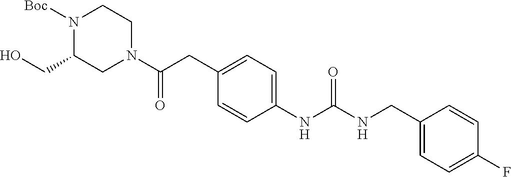 [SMILES: CC(C)(C)OC(=O)N1CCN(C(=O)Cc2ccc(NC(=O)NCc3ccc(F)cc3)cc2)C[C@@H]1CO] |
| | | | Compound 153. tert-butyl (2R)-4-{2-[4-({[(4-fluorophenyl) |
| | | | methyl]amino}carbonylamino)phenyl]acetyl}-2- |
| | | | (hydroxymethyl)piperazinecarboxylate. LCMS- |
| | | | ESI (POS.) m/z: 501.20 (M + H)+. ¹H NMR (400 |
| | | | MHz, DMSO-d₆) δ 8.52 (d, J = 4.5 Hz, 1H), 7.39- |
| | | | 7.27 (m, 4H), 7.16 (t, J = 8.7 Hz, 2H), 7.07 (dd, J = |
| | | | 15.9, 8.1 Hz, 2H), 6.60 (s, 1H), 5.01 (t, J = 5.3 Hz, |
| | | | 1H), 4.73-4.63 (m, 1H), 4.33 (d, J = 14.0 Hz, 1H), |
| | | | 4.27 (d, J = 5.9 Hz, 2H), 4.20 (d, J = 13.0 Hz, 1H), |
| | | | 4.11-3.78 (m, 3H), 3.77-3.51 (m, 2H), 3.29-2.95 |
| | | | (m, 1H), 2.92-2.61 (m, 2H), 1.40 (s, 9H). |
| 230 | 1.1 | (R)- pyrrolidin-2- ylmethanol | 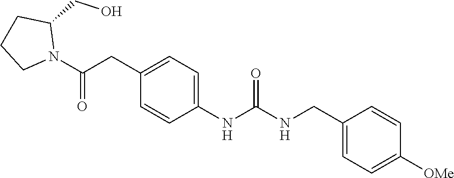 [SMILES: COc1ccc(CNC(=O)Nc2ccc(CC(=O)N3CCC[C@@H]3CO)cc2)cc1] |
| | | | Compound 230. [(4-{2-[(2R)-2-(hydroxymethyl) |
| | | | pyrrolidinyl]-2-oxoethyl}phenyl)amino]-N-[(4- |
| | | | methoxyphenyl)methyl]carboxamide. LCMS-ESI |
| | | | (POS.) m/z: 398.20 (M + H)+. ¹H NMR (400 MHz, |
| | | | DMSO-d₆) δ 8.43 (s, 1H), 7.35-7.27 (m, 2H), 7.23 |
| | | | (d, J = 8.2 Hz, 2H), 7.08 (dd, J = 8.4, 2.7 Hz, 2H), |
| | | | 6.90 (d, J = 8.2 Hz, 2H), 6.48 (t, J = 6.0 Hz, 1H), |
| | | | 4.22 (d, J = 5.7 Hz, 2H), 4.10 (q, J = 5.3 Hz, 1H), |
| | | | 3.96 (d, J = 19.8 Hz, 1H), 3.74 (s, 3H), 3.61-3.39 |
| | | | (m, 4H), 3.30-3.14 (m, 2H), 1.82 (dt, J = 23.9, 9.6 Hz, 4H). |
| 248 | 1.1 | (S)- pyrrolidin-3- ylmethanol | 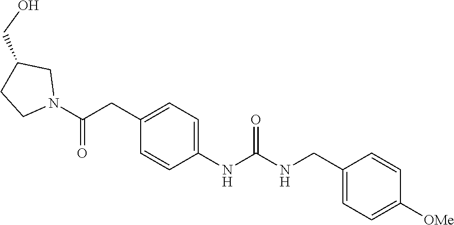 [SMILES: COc1ccc(CNC(=O)Nc2ccc(CC(=O)N3CC[C@H](CO)C3)cc2)cc1] |
| | | | Compound 248. [(4-{2-[(3S)-3-(hydroxymethyl) |
| | | | pyrrolidinyl]-2-oxoethyl}phenyl)amino]-N-[(4- |
| | | | methoxyphenyl)methyl]carboxamide. LCMS-ESI |
| | | | (POS.) m/z: 398.20 (M + H)+. ¹H NMR (400 MHz, |
| | | | DMSO-d₆) δ 8.44 (s, 1H), 7.31 (d, J = 8.1 Hz, 2H), |
| | | | 7.23 (d, J = 8.1 Hz, 2H), 7.07 (d, J = 8.1 Hz, 2H), |
| | | | 6.90 (d, J = 8.1 Hz, 2H), 6.49 (t, J = 5.8 Hz, 1H), |
| | | | 4.73-4.62 (m, 1H), 4.22 (d, J = 5.8 Hz, 2H), 3.74 |
| | | | (d, J = 1.2 Hz, 3H), 3.62-3.47 (m, 3H), 3.41 (dq, |
| | | | J = 11.8, 6.7, 5.2 Hz, 2H), 3.28-3.14 (m, 2H), 3.05 |
| | | | (dd, J = 11.8, 6.8 Hz, 1H), 2.39-2.12 (m, 1H), 1.88 |
| | | | (dd, J = 34.6, 6.6 Hz, 1H), 1.74-1.48 (m, 1H). |
| 277 | 1.1 | (R)- pyrrolidin-3- ylmethanol | 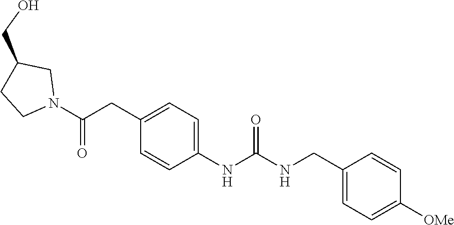 [SMILES: COc1ccc(CNC(=O)Nc2ccc(CC(=O)N3CC[C@@H](CO)C3)cc2)cc1] |
| | | | Compound 277. [(4-{2-[(3R)-3-(hydroxymethyl) |
| | | | pyrrolidinyl]-2-oxoethyl}phenyl)amino]-N-[(4- |
| | | | methoxyphenyl)methyl]carboxamide. LCMS-ESI |
| | | | (POS.) m/z: 398.20 (M + H)+. ¹H NMR (400 MHz, |
| | | | DMSO-d₆) δ 8.43 (s, 1H), 7.32 (d, J = 8.1 Hz, 2H), |
| | | | 7.23 (d, J = 8.2 Hz, 2H), 7.07 (d, J = 8.2 Hz, 2H), |
| | | | 6.90 (d, J = 8.2 Hz, 2H), 6.48 (t, J = 5.9 Hz, 1H), |
| | | | 4.68 (s, 1H), 4.22 (d, J = 5.7 Hz, 2H), 3.74 (d, J = 1.2 |
| | | | Hz, 3H), 3.62-3.47 (m, 3H), 3.47-3.28 (m, 3H), |
| | | | 3.22 (ddd,J = 22.2, 11.0, 7.5 Hz, 1H), 3.05 (dd, J = |
| | | | 11.9, 6.9 Hz, 1H), 2.27 (dp, J = 43.0, 7.2 Hz, 1H), |
| | | | 1.88 (ddt, J = 41.0, 12.3, 6.5 Hz, 1H), 1.73-1.48 (m, 1H). |
| 20 | 1.2 | (R)- pyrrolidin-2- ylmethanol | 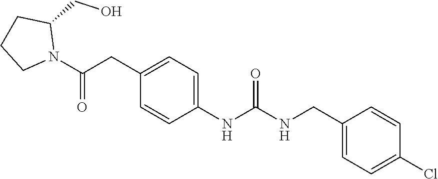 [SMILES: O=C(NCc1ccc(Cl)cc1)Nc1ccc(CC(=O)N2CCC[C@@H]2CO)cc1] |
| | | | Compound 20. [(4-{2-[(2R)-2-(hydroxymethyl) |
| | | | pyrrolidinyl]-2-oxoethyl}phenyl)amino]-N-[(4- |
| | | | chlorophenyl)methyl]carboxamide. LCMS-ESI |
| | | | (POS.) m/z: 403.10 (M + H)+. ¹H NMR (400 MHz, |
| | | | DMSO-d₆) δ 8.53 (s, 1H), 7.43-7.36 (m, 2H), 7.32 |
| | | | (d, J = 8.3 Hz, 4H), 7.08 (dd, J = 8.3, 2.7 Hz, 2H), |
| | | | 6.62 (t, J = 5.8 Hz, 1H), 4.96 (t, J = 5.7 Hz, 1H), 4.75 |
| | | | (t, J = 5.7 Hz, 1H), 4.28 (d, J = 5.9 Hz, 2H), 4.10 (q, |
| | | | J = 5.4 Hz, 1H), 3.96 (d, J = 17.9 Hz, 1H), 3.59- |
| | | | 3.48 (m, 2H), 3.43 (dd, J = 7.5, 4.8 Hz, 2H), 3.25 |
| | | | (dd, J = 10.5, 7.0 Hz, 1H), 3.18 (dd, J = 5.3, 1.3 Hz, |
| | | | 2H), 1.94-1.73 (m, 4H). |
| 157 | 1.3 | 3-azabicyclo [3.1.0]hexane | 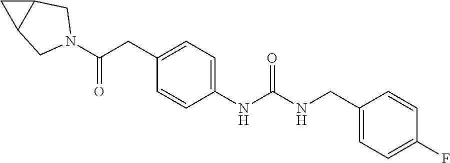 [SMILES: O=C(NCc1ccc(F)cc1)Nc1ccc(CC(=O)N2CC3CC3C2)cc1] |
| | | | Compound 157. N-{4-[2-(3-azabicyclo[3.1.0]hex- |
| | | | 3-yl)-2-oxoethyl]phenyl}{[(4- |
| | | | fluorophenyl)methyl]amino}carboxamide. LCMS- |
| | | | ESI (POS.) m/z: 368.05 (M + H)+. ¹H NMR (400 |
| | | | MHz, DMSO-d₆) δ 8.50 (s, 1H), 7.37-7.26 (m, 4H), |
| | | | 7.15 (t, J = 8.7 Hz, 2H), 7.04 (d, J = 8.2 Hz, 2H), |
| | | | 6.59 (t, J = 5.9 Hz, 1H), 4.27 (d, J = 5.9 Hz, 2H), |
| | | | 3.59 (t, J = 10.5 Hz, 2H), 3.48 (s, 3H), 3.23 (dd, J = |
| | | | 11.7, 4.3 Hz, 1H), 1.53 (ddd,J = 23.3, 7.9, 4.0 Hz, |
| | | | 2H), 0.66 (q, J = 7.4 Hz, 1H),-0.01 (q, J = 4.4 Hz, |
| | | | 1H). |
| 178 | 1.3 | 2-methyl- 2,6- diazaspiro [3.4]octane | 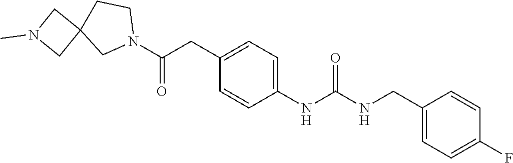 [SMILES: CN1CC2(CCN(C(=O)Cc3ccc(NC(=O)NCc4ccc(F)cc4)cc3)C2)C1] |
| | | | Compound 178. {[(4- |
| | | | fluorophenyl)methyl]amino}-N-{4-[2-(2-methyl- |
| | | | 2,6-diazaspiro[3.4]oct-6-yl)-2- |
| | | | oxoethyl]phenyl}carboxamide. LCMS-ESI (POS.) |
| | | | m/z: 411.05 (M + H)+. ¹H NMR (400 MHz, |
| | | | Methanol-d₄) δ 8.43 (s, 1H), 7.41-7.30 (m, 4H), |
| | | | 7.27 (t, J = 6.8 Hz, 1H), 7.19 (dd, J = 17.6, 8.6 Hz, |
| | | | 2H), 7.05 (dt, J = 15.4, 8.5 Hz, 3H), 4.36 (d, J = 12.9 |
| | | | Hz, 3H), 4.12-3.92 (m, 2H), 3.84-3.56 (m, 3H), |
| | | | 3.50 (s, 2H), 2.87 (d, J = 14.7 Hz, 2H), 2.28 (t, J = |
| | | | 7.0 Hz, 1H), 2.20 (t, J = 7.2 Hz, 1H), 1.32 (s, 2H). |
| 163 | 1.3 | 7-methyl- 2,7- diazaspiro [3.5] nonane | 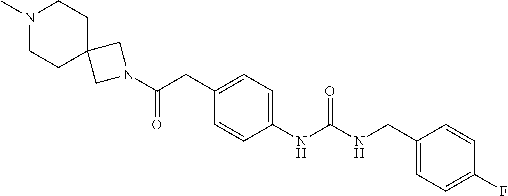 [SMILES: CN1CCC2(CC1)CN(C(=O)Cc1ccc(NC(=O)NCc3ccc(F)cc3)cc1)C2] |
| | | | Compound 163. {[(4- |
| | | | fluorophenyl)methyl]amino}-N-{4-[2-(7-methyl- |
| | | | 2,7-diazaspiro[3.5]non-2-yl)-2- |
| | | | oxoethyl]phenyl}carboxamide. LCMS-ESI (POS.) |
| | | | m/z: 425.10 (M + H)+. ¹H NMR (400 MHz, |
| | | | Methanol-d₄) δ 8.50 (s, 1H), 7.40-7.29 (m, 4H), |
| | | | 7.18 (d, J = 8.1 Hz, 2H), 7.12-7.02 (m, 2H), 4.38 |
| | | | (s, 2H), 4.00 (s, 2H), 3.78 (s, 2H), 3.48 (s, 2H), 3.04 |
| | | | (d, J = 26.7 Hz, 4H), 2.75 (s, 3H), 2.01 (t, J = 5.7 Hz, |
| | | | 4H), 1.31 (s, 1H). |
| 149 | 1.3 | 4-((3- methyliso- xazol-5-yl) methoxy) cyclohexan- 1-amine | 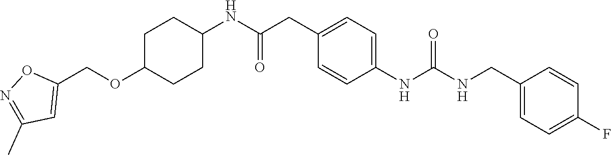 [SMILES: Cc1cc(COC2CCC(NC(=O)Cc3ccc(NC(=O)NCc4ccc(F)cc4)cc3)CC2)on1] |
| | | | Compound 149. 2-[4-({[(4- |
| | | | fluorophenyl)methyl]amino}carbonylamino) |
| | | | phenyl]-N-{4-[(3-methylisoxazol-5- |
| | | | yl)methoxy]cyclohexyl}acetamide. LCMS-ESI |
| | | | (POS.) m/z: 605.10 (M + H)+. ¹H NMR (400 MHz, |
| | | | Methanol-d₄) δ 7.40-7.27 (m, 4H), 7.19 (d, J = 8.1 |
| | | | Hz, 2H), 7.07 (t, J = 8.5 Hz, 2H), 6.26 (s, 1H), 3.64 |
| | | | (s, 2H), 2.29 (s, 4H), 2.14-1.98 (m, 3H), 1.97- |
| | | | 1.87 (m, 2H), 1.45-1.21 (m, 8H), 0.98-0.84 (m, 2H). |
| 162 | 1.3 | 3-azabicyclo [3.1.0] hexan-6-ol | 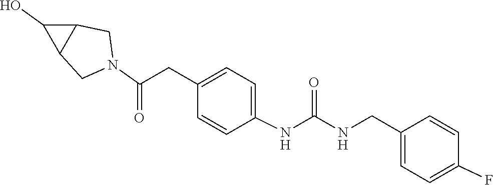 [SMILES: O=C(NCc1ccc(F)cc1)Nc1ccc(CC(=O)N2CC3C(O)C3C2)cc1] |
| | | | Compound 162. {[(4-fluorophenyl)methyl]amino}- |
| | | | N-{4-[2-(6-hydroxy-3-azabicyclo[3.1.0]hex-3-yl)-2- |
| | | | oxoethyl]phenyl}carboxamide. LCMS-ESI (POS.) |
| | | | m/z: 384.10 (M + H)+. ¹H NMR (400 MHz, |
| | | | Methanol-d₄) δ 7.40-7.31 (m, 4H), 7.14 (d, J = 8.1 |
| | | | Hz, 2H), 7.07 (t, J = 8.6 Hz, 2H), 4.38 (s, 2H), 3.70 |
| | | | (d, J = 11.4 Hz, 2H), 3.60 (d, J = 11.6 Hz, 3H), 3.44 |
| | | | (dd, J = 12.3, 4.8 Hz, 1H), 3.37 (s, 2H), 2.95 (s, 1H), |
| | | | 1.72 (p, J = 8.2 Hz, 2H), 1.31 (s, 1H). |
| 158 | 1.3 | tert-butyl ((1R,5S,6s)- 3-azabicyclo [3.1.0]hexan- 6-yl) carbamate | 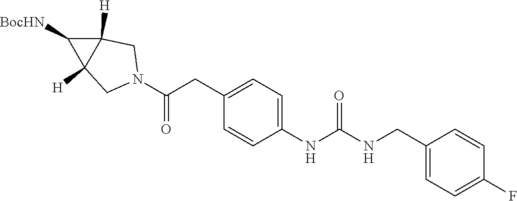 [SMILES: CC(C)(C)OC(=O)N[C@H]1[C@@H]2CN(C(=O)Cc3ccc(NC(=O)NCc4ccc(F)cc4)cc3)C[C@@H]21] |
| | | | Compound 158. N-[4-(2-{(5S,1R)-6-[(tert- |
| | | | butoxy)carbonylamino]-3-azabicyclo[3.1.0]hex-3- |
| | | | yl}-2-oxoethyl)phenyl]{[(4- |
| | | | fluorophenyl)methyl]amino}carboxamide. LCMS- |
| | | | ESI (POS.) m/z: 383.10 (M + H)+. ¹H NMR (400 |
| | | | MHz, DMSO-d₆) δ 8.51 (s, 1H), 7.39-7.27 (m, 4H), |
| | | | 7.16 (t, J = 8.7 Hz, 2H), 7.06 (t, J = 11.3 Hz, 3H), |
| | | | 6.60 (t, J = 6.0 Hz, 1H), 4.27 (d, J = 5.8 Hz, 2H), |
| | | | 3.73-3.39 (m, 5H), 2.07 (s, 1H), 1.64 (d, J = 25.7 |
| | | | Hz, 2H), 1.38 (s, 9H), 1.25 (s, 1H). |
| 240 | 1.1 | (R)-4,4- difluoropy- rrolidine-2- carboxamide | 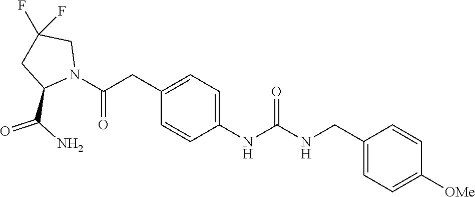 [SMILES: COc1ccc(CNC(=O)Nc2ccc(CC(=O)N3CC(F)(F)C[C@@H]3C(N)=O)cc2)cc1] |
| | | | Compound 240. (2R)-4,4-difluoro-1-{2-[4-({[(4- |
| | | | methoxyphenyl)methyl]amino}carbonylamino) |
| | | | phenyl]acetyl} pyrrolidine-2-carboxamide. LCMS- |
| | | | ESI (POS.) m/z: 447.10 (M + H)+. ¹H NMR (400 |
| | | | MHz, DMSO-d₆) δ 8.45 (s, 1H), 7.43 (s, 1H), 7.38- |
| | | | 7.29 (m, 2H), 7.23 (d, J = 8.1 Hz, 2H), 7.08 (dt, J = |
| | | | 16.0, 7.6 Hz, 2H), 6.93-6.82 (m, 2H), 6.49 (s, 1H), |
| | | | 4.45 (d, J = 7.8 Hz, 1H), 4.22 (d, J = 5.8 Hz, 2H), |
| | | | 4.02 (ddd, J = 41.0, 29.6, 13.3 Hz, 1H), 3.74 (d, J = |
| | | | 1.6 Hz, 2H), 3.59 (s, 1H), 3.47 (d, J = 9.7 Hz, 1H), |
| | | | 3.18 (d, J = 5.2 Hz, 1H), 2.98 (s, 1H), 2.82 (s, 1H), |
| | | | 2.75-2.65 (m, 1H), 2.35 (d, J = 11.9 Hz, 1H). |
| 198 | 1.1 | 4-((3- methyliso- xazol-5-yl) methoxy) cyclohexan- 1-amine | 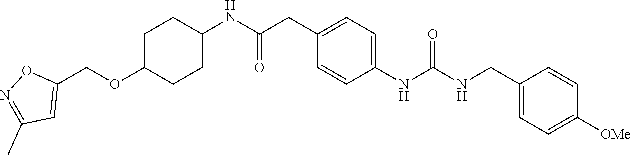 [SMILES: COc1ccc(CNC(=O)Nc2ccc(CC(=O)NC3CCC(OCc4cc(C)no4)CC3)cc2)cc1] |
| | | | Compound 198. 2-[4-({N-[(4-methoxyphenyl)methyl] |
| | | | carbamoyl} amino)phenyl]-N-{4-[(3-methylisoxazol-5- |
| | | | yl)methoxy]cyclohexyl}acetamide. LCMS-ESI |
| | | | (POS.) m/z: 508.20 (M + H)+. ¹H NMR (400 MHz, |
| | | | DMSO-d₆) δ 8.43 (s, 1H), 7.85 (d, J = 7.8 Hz, 1H), |
| | | | 7.30 (d, J = 8.0 Hz, 2H), 7.23 (d, J = 8.0 Hz, 2H), |
| | | | 7.09 (d, J = 8.1 Hz, 2H), 6.90 (d, J = 8.1 Hz, 2H), |
| | | | 6.50 (s, 1H), 6.32 (s, 1H), 4.57 (s, 2H), 4.21 (d, J = |
| | | | 5.7 Hz, 2H), 3.74 (d, J = 1.5 Hz, 3H), 3.49 (s, 1H), |
| | | | 3.27 (s, 2H), 3.18 (d, J = 5.2 Hz, 1H), 2.22 (d, J = 1.4 |
| | | | Hz, 3H), 1.97 (d, J = 10.6 Hz, 2H), 1.78 (d, J = 11.0 |
| | | | Hz, 2H), 1.21 (q, J = 12.7, 12.2 Hz, 4H). |
| 235 | 1.1 | 3-azabicyclo [3.1.0] hexane | 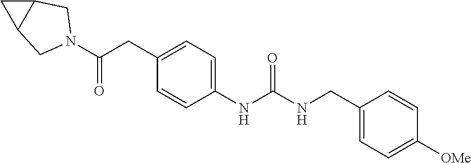 [SMILES: COc1ccc(CNC(=O)Nc2ccc(CC(=O)N3CC4CC4C3)cc2)cc1] |
| | | | Compound 235. ({4-[2-(3-azabicyclo[3.1.0]hex-3- |
| | | | yl)-2-oxoethyl]phenyl}amino)-N-[(4- |
| | | | methoxyphenyl)methyl]carboxamide. LCMS-ESI |
| | | | (POS.) m/z: 380.15 (M + H)+. ¹H NMR (400 MHz, |
| | | | DMSO-d₆) δ 8.44 (s, 1H), 7.37-7.26 (m, 2H), 7.26- |
| | | | 7.18 (m, 2H), 7.04 (d, J = 8.1 Hz, 2H), 6.94-6.85 |
| | | | (m, 2H), 6.49 (t, J = 5.8 Hz, 1H), 4.21 (d, J = 5.8 Hz, |
| | | | 2H), 3.73 (d, J = 1.4 Hz, 4H), 3.59 (t, J = 10.6 Hz, |
| | | | 2H), 3.47 (d, J = 9.4 Hz, 4H), 3.23 (dd, J = 11.8, 4.4 |
| | | | Hz, 1H), 1.61-1.41 (m, 3H), 0.66 (q, J = 7.4 Hz, |
| | | | 1H), -0.00 (q, J = 4.4 Hz, 1H). |
| 261 | 1.1 | 2-methyl- 2,6- diazaspiro [3.4]octane | 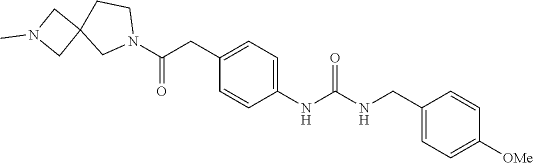 [SMILES: COc1ccc(CNC(=O)Nc2ccc(CC(=O)N3CCC4(CN(C)C4)C3)cc2)cc1] |
| | | | Compound 261. N-[(4- |
| | | | methoxyphenyl)methyl]({4-[2-(2-methyl-2,6- |
| | | | diazaspiro[3.4]oct-6-yl)-2- |
| | | | oxoethyl]phenyl}amino)carboxamide. LCMS-ESI |
| | | | (POS.) m/z: 424.15 (M + H)+. ¹H NMR (400 MHz, |
| | | | DMSO-d₆) δ 8.44 (s, 1H), 7.32 (d, J = 8.3 Hz, 2H), |
| | | | 7.23 (d, J = 8.1 Hz, 2H), 7.07 (d, J = 8.2 Hz, 2H), |
| | | | 6.90 (d, J = 8.1 Hz, 2H), 6.49 (s, 1H), 4.22 (d, J = 5.6 |
| | | | Hz, 2H), 4.18-3.92 (m, 4H), 3.74 (s, 3H), 3.69 (d, J |
| | | | = 8.4 Hz, 2H), 3.52 (d, J = 14.2 Hz, 4H), 3.18 (s, |
| | | | 3H), 2.85 (t, J = 5.9 Hz, 3H), 2.19 (t, J = 6.5 Hz, |
| | | | 1H), 2.09 (t, J = 7.1 Hz, 1H). |
| 216 | 1.1 | 3-azabicyclo [3.1.0] hexan-6-ol | 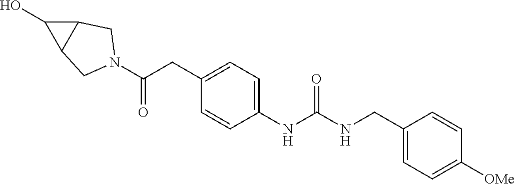 [SMILES: COc1ccc(CNC(=O)Nc2ccc(CC(=O)N3CC4C(O)C4C3)cc2)cc1] |
| | | | Compound 216. ({4-[2-(6-hydroxy-3- |
| | | | azabicyclo[3.1.0]hex-3-yl)-2- |
| | | | oxoethyl]phenyl}amino)-N-[(4- |
| | | | methoxyphenyl)methyl]carboxamide. LCMS-ESI |
| | | | (POS.) m/z: 396.10 (M + H)+. ¹H NMR (400 MHz, |
| | | | DMSO-d₆) δ 8.45 (s, 1H), 7.31 (d, J = 8.1 Hz, 2H), |
| | | | 7.23 (d, J = 8.1 Hz, 2H), 7.03 (d, J = 8.1 Hz, 2H), |
| | | | 6.93-6.87 (m, 2H), 6.49 (t, J = 5.9 Hz, 1H), 5.43 (s, |
| | | | 1H), 4.22 (d, J = 5.8 Hz, 2H), 4.10 (q, J = 5.6 Hz, |
| | | | 1H), 3.74 (d, J = 1.3 Hz, 3H), 3.64-3.46 (m, 4H), |
| | | | 3.45 (s, 2H), 3.28 (dd, J = 12.0, 5.2 Hz, 1H), 3.18 (d, |
| | | | J = 5.2 Hz, 1H), 2.82 (s, 1H), 1.68-1.50 (m, 2H). |
| 269 | 1.1 | (S)-octa- hydropyrrolo [1,2-a] pyrazine | 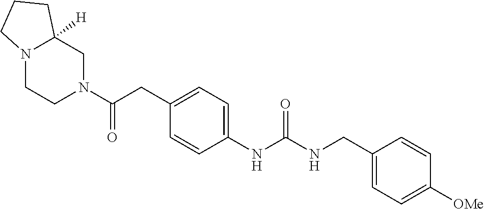 [SMILES: COc1ccc(CNC(=O)Nc2ccc(CC(=O)N3CCN4CCC[C@H]4C3)cc2)cc1] |
| | | | Compound 269. ({4-[2-((1S)-3,6-diazabicyclo |
| | | | [4.3.0]non-3-yl)-2-oxoethyl]phenyl}amino)-N-[(4- |
| | | | methoxyphenyl)methyl]carboxamide. LCMS-ESI |
| | | | (POS.) m/z: 424.20 (M + H)+. ¹HNMR (400 MHz, |
| | | | DMSO-d₆) δ 8.44 (s, 1H), 8.15 (s, 1H), 7.32 (d, J = |
| | | | 8.1 Hz, 2H), 7.23 (d, J = 8.1 Hz, 2H), 7.07 (d, J = 8.1 |
| | | | Hz, 2H), 6.90 (d, J = 8.1 Hz, 2H), 6.49 (t, J = 5.9 Hz, |
| | | | 1H), 4.49 (d, J = 12.4 Hz, 1H), 4.35 (d, J = 13.1 Hz, |
| | | | 1H), 4.22 (d, J = 5.8 Hz, 2H), 4.15-4.00 (m, 1H), |
| | | | 3.89 (d, J = 13.6 Hz, 1H), 3.74 (s, 2H), 3.63 (s, 2H), |
| | | | 2.96 (td, J = 24.8, 23.3, 13.6 Hz, 3H), 2.82-2.56 |
| | | | (m, 1H), 2.38-2.23 (m, 1H), 2.09-1.82 (m, 1H), |
| | | | 1.81-1.58 (m, 2H), 1.35-1.20 (m, 1H). |
| 189 | 1.3 | (S)-octa- hydropyrrolo [1,2-a] pyrazine | 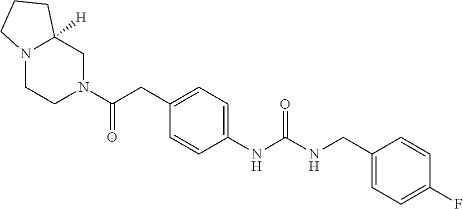 [SMILES: O=C(NCc1ccc(F)cc1)Nc1ccc(CC(=O)N2CCN3CCC[C@H]3C2)cc1] |
| | | | Compound 189. N-{4-[2-((1S)-3,6- |
| | | | diazabicyclo[4.3.0]non-3-yl)-2-oxoethyl]phenyl}{[(4- |
| | | | fluorophenyl)methyl]amino}carboxamide. LCMS- |
| | | | ESI (POS.) m/z: 411.10 (M + H)+. ¹H NMR (400 |
| | | | MHz, Methanol-d₄) δ 8.26 (s, 1H), 7.42-7.30 (m, |
| | | | 3H), 7.19 (d, J = 8.1 Hz, 2H), 7.07 (t, 3H), 4.53- |
| | | | 4.42 (m, 1H), 4.38 (s, 1H), 4.32 (d, J = 14.3 Hz, 1H), |
| | | | 4.14 (dd, J = 15.6, 11.3 Hz, 1H), 3.97 (d, J = 14.7 |
| | | | Hz, 1H), 3.78 (s, 2H), 3.51 (t, J = 12.2 Hz, 1H), 3.30- |
| | | | 3.13 (m, 2H), 3.13-3.02 (m, 1H), 3.00-2.78 (m, |
| | | | 2H), 2.72 (t, J = 11.1 Hz, 1H), 2.13 (dq, J = 12.5, 6.4 Hz, 1H), |
| | | | 2.02 (h, J = 7.4, 6.9 Hz, 2H), 1.65 (dq, J = 26.8, 10.2 Hz, 1H). |
| 309 | 1.1 | (R)-azetidin- 2-ylmethanol | 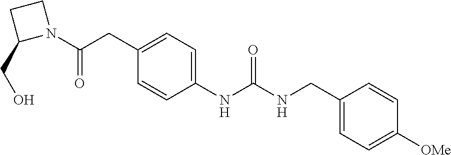 [SMILES: COc1ccc(CNC(=O)Nc2ccc(CC(=O)N3CC[C@@H]3CO)cc2)cc1] |
| | | | Compound 309. [(4-{2-[(2R)-2-(hydroxymethyl) |
| | | | azetidinyl]-2-oxoethyl}phenyl)amino]-N-[(4- |
| | | | methoxyphenyl)methyl]carboxamide. LCMS-ESI |
| | | | (POS.) m/z: 384.20 (M + H)+. ¹H NMR (400 MHz, |
| | | | DMSO-d₆) δ 8.44 (d, J = 6.5 Hz, 1H), 7.31 (t, J = 6.5 |
| | | | Hz, 2H), 7.26-7.20 (m, 2H), 7.08 (d, J = 8.3 Hz, |
| | | | 2H), 6.93-6.87 (m, 2H), 6.50 (d, J = 7.8 Hz, 1H), |
| | | | 5.09 (d, J = 5.5 Hz, 1H), 4.87 (t, J = 5.9 Hz, 1H), |
| | | | 4.45 (s, 1H), 4.29 (s, 1H), 4.22 (d, J = 5.7 Hz, 2H), |
| | | | 4.01 (s, 1H), 3.74 (d, J = 1.5 Hz, 3H), 3.72-3.59 |
| | | | (m, 2H), 3.54 (d, J = 11.2 Hz, 1H), 2.22 (dt, J = 16.7, |
| | | | 8.2 Hz, 1H), 1.99 (d, J = 34.9 Hz, 1H). |
| 183 | 1.3 | 5-methyl- 2-oxa-5,8- diazaspiro [3.5]nonane | 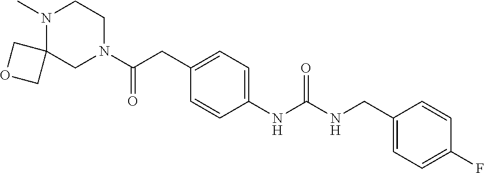 [SMILES: CN1CCN(C(=O)Cc2ccc(NC(=O)NCc3ccc(F)cc3)cc2)CC12COC2] |
| | | | Compound 183. {[(4-fluorophenyl) |
| | | | methyl]amino}-N-{4-[2-(9-methyl-2- |
| | | | oxa-6,9-diazaspiro[3.5]non-6-yl)-2- |
| | | | oxoethyl]phenyl}carboxamide. LCMS-ESI (POS.) |
| | | | m/z: 427.10 (M + H)+. ¹H NMR (400 MHz, DMSO- |
| | | | d₆) δ 8.52 (s, 1H), 7.39-7.28 (m, 4H), 7.22-7.10 |
| | | | (m, 3H), 7.06 (d, J = 8.1 Hz, 1H), 6.61 (t, J = 6.1 Hz, |
| | | | 1H), 4.61 (dd, J = 16.8, 6.7 Hz, 2H), 4.27 (d, J = 5.9 |
| | | | Hz, 2H), 4.06 (d, J = 6.6 Hz, 1H), 3.99 (d, J = 6.8 |
| | | | Hz, 1H), 3.69 (d, J = 10.6 Hz, 3H), 3.62 (s, 1H), |
| | | | 3.47-3.38 (m, 2H), 2.40 (d, J = 13.4 Hz, 3H), |
| | | | 2.36-2.32 (m, 1H), 2.28-2.20 (m, 1H). |
| 197 | 1.3 | (3R,4R)- pyrrolidine- 3,4-diol | 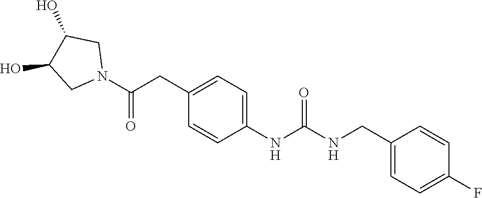 [SMILES: O=C(NCc1ccc(F)cc1)Nc1ccc(CC(=O)N2C[C@@H](O)[C@H](O)C2)cc1] |
| | | | Compound 197. N-{4-[2-((3R,4R)-3,4- |
| | | | dihydroxypyrrolidinyl)-2-oxoethyl]phenyl}{[(4- |
| | | | fluorophenyl)methyl]amino}carboxamide. LCMS- |
| | | | ESI (POS.) m/z: 388.10 (M + H)+. ¹H NMR (400 |
| | | | MHz, Methanol-d₄) δ 7.39-7.29 (m, 4H), 7.19 (d, |
| | | | J = 8.1 Hz, 2H), 7.07 (t, J = 8.6 Hz, 2H), 4.37 (s, 2H), |
| | | | 4.18-4.02 (m, 2H), 3.77 (dd, J = 11.5, 4.1 Hz, 1H), |
| | | | 3.67 (s, 2H), 3.62 (dd, J = 13.0, 4.2 Hz, 1H), 3.51 (d, |
| | | | J = 11.9 Hz, 2H). |
| 186 | 1.3 | (R)-azetidin- 2-ylmethanol | 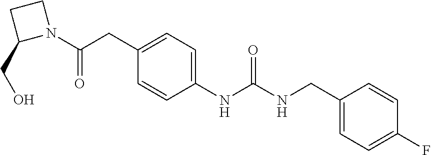 [SMILES: O=C(NCc1ccc(F)cc1)Nc1ccc(CC(=O)N2CC[C@@H]2CO)cc1] |
| | | | Compound 186. N-(4-{2-[(2R)-2- |
| | | | (hydroxymethyl)azetidinyl]-2-oxoethyl}phenyl){[(4- |
| | | | fluorophenyl)methyl]amino}carboxamide. LCMS- |
| | | | ESI (POS.) m/z: 372.10 (M + H)+. ¹H NMR (400 |
| | | | MHz, Methanol-d₄) δ 7.40-7.30 (m, 4H), 7.22- |
| | | | 7.15 (m, 2H), 7.11-7.02 (m, 2H), 4.61-4.52 (m, |
| | | | 1H), 4.51-4.44 (m, 1H), 4.37 (s, 2H), 4.11 (t, J = |
| | | | 7.8 Hz, 1H), 3.94-3.83 (m, 2H), 3.76 (dd, J = 12.3, |
| | | | 4.6 Hz, 1H), 3.68 (dd, J = 11.9, 4.7 Hz, 1H), 3.64- |
| | | | 3.47 (m, 1H), 3.44 (s, 1H), 2.46-2.24 (m, 1H), 2.23- |
| | | | 2.09 (m, 1H), 2.05 (s, 1H). |
| 185 | 1.3 | (S)-azetidin- 2-ylmethanol | 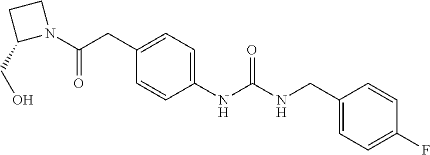 [SMILES: O=C(NCc1ccc(F)cc1)Nc1ccc(CC(=O)N2CC[C@H]2CO)cc1] |
| | | | Compound 185. N-(4-{2-[(2S)-2- |
| | | | (hydroxymethyl)azetidinyl]-2-oxoethyl}phenyl){[(4- |
| | | | fluorophenyl)methyl]amino}carboxamide. LCMS- |
| | | | ESI (POS.) m/z: 372.10 (M + H)+. ¹H NMR (400 |
| | | | MHz, Methanol-d₄) δ 7.37-7.29 (m, 4H), 7.18 (d, |
| | | | J = 8.0 Hz, 2H), 7.10-7.01 (m, 2H), 4.61-4.53 (m, |
| | | | 1H), 4.52-4.44 (m, 1H), 4.38 (s, 2H), 4.11 (t, J = |
| | | | 7.8 Hz, 1H), 3.94-3.84 (m, 1H), 3.76 (dd, J = 12.3, |
| | | | 4.7 Hz, 1H), 3.68 (dd, J = 11.9, 4.8 Hz, 1H), 3.64- |
| | | | 3.47 (m, 1H), 3.44 (s, 1H), 2.46-2.23 (m, 1H), 2.21- |
| | | | 2.08 (m, 1H), 2.04 (s, 1H). |
| 298 | 1.1 | (S)-azetidin- 2-ylmethanol | 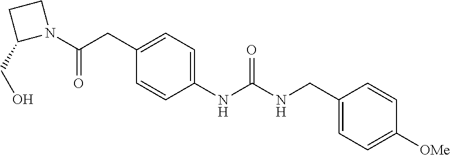 [SMILES: COc1ccc(CNC(=O)Nc2ccc(CC(=O)N3CC[C@H]3CO)cc2)cc1] |
| | | | Compound 298. [(4-{2-[(2S)-2- |
| | | | (hydroxymethyl)azetidinyl]-2- |
| | | | oxoethyl}phenyl)amino]-N-[(4- |
| | | | methoxyphenyl)methyl]carboxamide. LCMS-ESI |
| | | | (POS.) m/z: 384.20 (M + H)+. ¹H NMR (400 MHz, |
| | | | DMSO-d₆) δ 8.43 (d, J = 6.5 Hz, 1H), 7.31 (t, J = 6.8 |
| | | | Hz, 2H), 7.23 (d, J = 8.1 Hz, 2H), 7.08 (d, J = 8.1 |
| | | | Hz, 2H), 6.94-6.85 (m, 2H), 6.48 (s, 1H), 5.08 (s, |
| | | | 1H), 4.91-4.84 (m, 1H), 4.45 (s, 1H), 4.29 (s, 1H), |
| | | | 4.22 (d, J = 5.7 Hz, 2H), 4.01 (s, 1H), 3.74 (d, J = 1.6 |
| | | | Hz, 3H), 3.68-3.48 (m, 2H), 2.31-1.91 (m, 3H). |
| 245 | 1.1 | tert-butyl ((1R,5S,6s)- 3-azabicyclo [3.1.0] hexan-6-yl) carbamate | 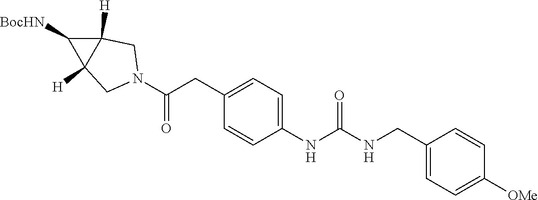 [SMILES: COc1ccc(CNC(=O)Nc2ccc(CC(=O)N3C[C@@H]4[C@H](C3)[C@H]4NC(=O)OC(C)(C)C)cc2)cc1] |
| | | | Compound 245. {[4-(2-{(5S,1R)-6-[(tert- |
| | | | butoxy)carbonylamino]-3-azabicyclo[3.1.0]hex-3- |
| | | | yl}-2-oxoethyl)phenyl]amino}-N-[(4- |
| | | | methoxyphenyl)methyl]carboxamide. LCMS-ESI |
| | | | (POS.) m/z: 495.10 (M + H)+. ¹H NMR (400 MHz, |
| | | | DMSO-d₆) δ 8.44 (s, 1H), 7.35-7.28 (m, 2H), 7.26- |
| | | | 7.19 (m, 2H), 7.04 (d, J = 6.5 Hz, 2H), 6.90 (d, J = |
| | | | 7.8 Hz, 2H), 6.48 (s, 1H), 4.21 (d, J = 5.1 Hz, 2H), |
| | | | 3.73 (t, J = 2.3 Hz, 4H), 3.58 (d, J = 10.7 Hz, 4H), 3.46 |
| | | | (s, 2H), 3.25 (d, J = 10.7 Hz, 2H), 1.95 (s, 4H), 1.54 (s, 9H). |
| 255 | 1.1 | 5-methyl-2- oxa-5,8- diazaspiro [3.5]nonane | 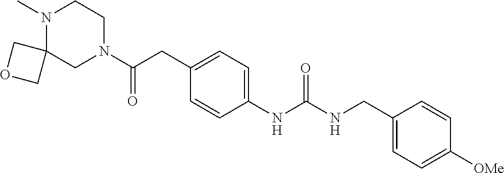 [SMILES: COc1ccc(CNC(=O)Nc2ccc(CC(=O)N3CCN(C)C4(COC4)C3)cc2)cc1] |
| | | | Compound 255. N-[(4- |
| | | | methoxyphenyl)methyl]({4-[2-(9-methyl-2-oxa- |
| | | | 6,9-diazaspiro[3.5]non-6-yl)-2- |
| | | | oxoethyl]phenyl}amino)carboxamide. LCMS-ESI |
| | | | (POS.) m/z: 440.20 (M + H)+. ¹H NMR (400 MHz, |
| | | | DMSO-d₆) δ 8.45 (s, 1H), 7.33 (d, J = 7.8 Hz, 2H), |
| | | | 7.23 (d, J = 8.1 Hz, 2H), 7.13 (d, J = 8.1 Hz, 1H), |
| | | | 7.06 (d, J = 8.1 Hz, 1H), 6.94-6.85 (m, 2H), 6.49 (t, |
| | | | J = 6.0 Hz, 1H), 4.61 (dd, J = 16.8, 6.8 Hz, 2H), 4.22 |
| | | | (d, J = 5.7 Hz, 2H), 4.08 (dd, J = 15.2, 6.0 Hz, 2H), |
| | | | 3.99 (d, J = 6.8 Hz, 1H), 3.74 (d, J = 1.4 Hz, 3H), |
| | | | 3.69 (d, J = 10.2 Hz, 2H), 3.62 (s, 1H), 3.18 (d, J = |
| | | | 4.6 Hz, 2H), 2.38 (t, J = 16.1 Hz, 4H), 2.24 (s, 1H). |
| 330 | 1.1 | pyrimidin-4- ylmethana- mine | 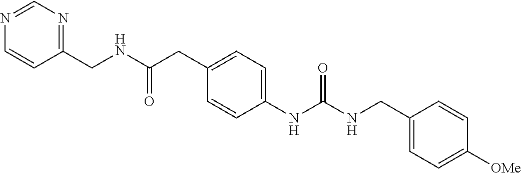 [SMILES: COc1ccc(CNC(=O)Nc2ccc(CC(=O)NCc3ccncn3)cc2)cc1] |
| | | | Compound 330. 2-[4-({N-[(4- |
| | | | methoxyphenyl)methyl]carbamoyl} amino)phenyl]- |
| | | | N-(pyrimidin-4-ylmethyl)acetamide. LCMS-ESI |
| | | | (POS.) m/z: 406.10 (M + H)+. ¹H NMR (400 MHz, |
| | | | DMSO-d₆) δ 9.10 (s, 1H), 8.72 (d, J = 5.4 Hz, 1H), |
| | | | 8.62 (s, 1H), 8.47 (s, 1H), 7.33 (t, J = 8.1 Hz, 3H), |
| | | | 7.23 (d, J = 8.1 Hz, 2H), 7.15 (d, J = 8.2 Hz, 2H), |
| | | | 6.90 (d, J = 8.1 Hz, 2H), 6.51 (s, 1H), 4.34 (d, J = 5.8 |
| | | | Hz, 2H), 4.22 (d, J = 5.9 Hz, 2H), 3.74 (s, 3H), 3.45 (s, 2H). |
| 343 | 1.1 | (3S,4S)- pyrrolidine- 3,4-diol | 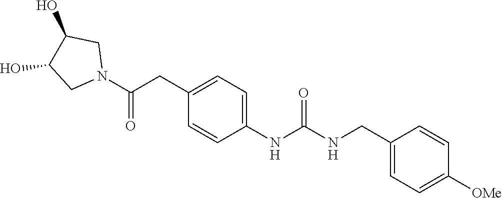 [SMILES: COc1ccc(CNC(=O)Nc2ccc(CC(=O)N3C[C@H](O)[C@@H](O)C3)cc2)cc1] |
| | | | Compound 343. ({4-[2-((3S,4S)-3,4- |
| | | | dihydroxypyrrolidinyl)-2-oxoethyl]phenyl}amino)-N-[(4- |
| | | | methoxyphenyl)methyl]carboxamide. LCMS-ESI |
| | | | (POS.) m/z: 400.10 (M + H)+. ¹H NMR (400 MHz, |
| | | | DMSO-d₆) δ 8.44 (s, 1H), 7.31 (d, J = 8.1 Hz, 2H), |
| | | | 7.23 (d, J = 8.0 Hz, 2H), 7.08 (d, J = 8.1 Hz, 2H), |
| | | | 6.90 (d, J = 8.1 Hz, 2H), 6.48 (t, J = 6.0 Hz, 1H), |
| | | | 5.16 (d, J = 3.5 Hz, 1H), 5.08 (d, J = 3.3 Hz, 1H), |
| | | | 4.22 (d, J = 5.8 Hz, 2H), 3.96 (s, 1H), 3.89 (s, 1H), |
| | | | 3.74 (d, J = 1.4 Hz, 3H), 3.68-3.56 (m, 1H), 3.35 |
| | | | (d, J = 5.8 Hz, 1H), 3.28 (d, J = 13.4 Hz, 2H), 3.18 |
| | | | (d, J = 5.4 Hz, 1H). |
| 68 | 1.2 | (R)-azetidin- 2-ylmethanol | 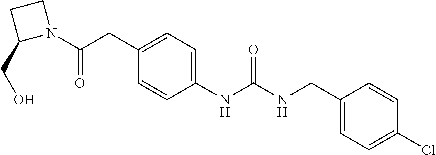 [SMILES: O=C(NCc1ccc(Cl)cc1)Nc1ccc(CC(=O)N2CC[C@@H]2CO)cc1] |
| | | | Compound 68. N-(4-{2-[(2R)-2- |
| | | | (hydroxymethyl)azetidinyl]-2-oxoethyl}phenyl){[(4- |
| | | | chlorophenyl)methyl]amino}carboxamide. LCMS- |
| | | | ESI (POS.) m/z: 388.00 (M + H)+. ¹H NMR (400 |
| | | | MHz, DMSO-d₆) δ 8.53 (d, J = 6.5 Hz, 1H), 7.40 (d, |
| | | | J = 8.1 Hz, 2H), 7.32 (d, J = 8.0 Hz, 4H), 7.09 (d, J = |
| | | | 8.1 Hz, 2H), 6.62 (s, 1H), 5.08 (t, J = 5.4 Hz, 1H), |
| | | | 4.87 (t, J = 5.7 Hz, 1H), 4.45 (d, J = 5.8 Hz, 1H), |
| | | | 4.28 (d, J = 6.1 Hz, 2H), 4.06-3.95 (m, 1H), 3.78- |
| | | | 3.60 (m, 2H), 3.58-3.51 (m, 1H), 3.30 (s, 1H), 2.32- |
| | | | 2.13 (m, 1H), 2.09-1.88 (m, 1H). |
| 63 | 1.2 | (S)-azetidin- 2-ylmethanol | 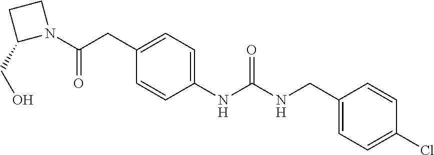 [SMILES: O=C(NCc1ccc(Cl)cc1)Nc1ccc(CC(=O)N2CC[C@H]2CO)cc1] |
| | | | Compound 63. N-(4-{2-[(2S)-2- |
| | | | (hydroxymethyl)azetidinyl]-2-oxoethyl}phenyl){[(4- |
| | | | chlorophenyl)methyl]amino}carboxamide. LCMS- |
| | | | ESI (POS.) m/z: 388.00 (M + H)+. ¹H NMR (400 |
| | | | MHz, DMSO-d₆) δ 8.54 (d, J = 6.6 Hz, 1H), 7.40 (d, |
| | | | J = 8.0 Hz, 2H), 7.33 (d, J = 7.9 Hz, 4H), 7.09 (d, J = |
| | | | 8.1 Hz, 2H), 6.64 (d, J = 7.0 Hz, 1H), 5.08 (t, J = 5.6 |
| | | | Hz, 1H), 4.87 (t, J = 5.7 Hz, 1H), 4.45 (dd, J = 8.9, |
| | | | 4.6 Hz, 1H), 4.28 (d, J = 5.9 Hz, 2H), 4.01 (q, J = |
| | | | 6.7, 4.7 Hz, 1H), 3.77-3.59 (m, 2H), 3.58-3.49 |
| | | | (m, 1H), 3.30(s, 1H), 2.32-2.12 (m, 1H), 2.08-1.89 (m, 1H). |
| 106 | 1.2 | tert-butyl- 2,6- diazaspiro [3.3]heptane- 2- carboxylate | 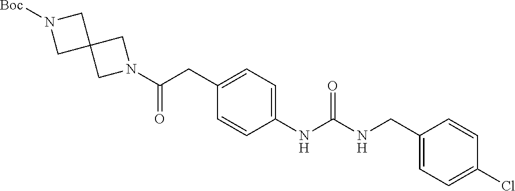 [SMILES: CC(C)(C)OC(=O)N1CC2(CN(C(=O)Cc3ccc(NC(=O)NCc4ccc(Cl)cc4)cc3)C2)C1] |
| | | | Compound 106. tert-butyl 6-{2-[4-({[(4- |
| | | | chlorophenyl)methyl]amino}carbonylamino) |
| | | | phenyl]acetyl}-2,6-diazaspiro[3.3]heptane-2- |
| | | | carboxylate. LCMS-ESI (POS.) m/z: 500.05 |
| | | | (M + H)+. ¹H NMR (400 MHz, DMSO-d₆) δ 8.58 (s, |
| | | | 1H), 7.40 (d, J = 8.1 Hz, 2H), 7.32 (d, J = 8.2 Hz, |
| | | | 4H), 7.06 (d, J = 8.1 Hz, 2H), 6.67 (s, 1H), 4.28 (d, |
| | | | J = 6.9 Hz, 4H), 3.97 (s, 6H), 3.29 (s, 2H), 1.38 (s, |
| | | | 8H), 0.96 (d, J = 6.6 Hz, 1H). |
| 73 | 1.2 | 5-methyl-2- oxa-5,8- diazaspiro [3.5]nonane | 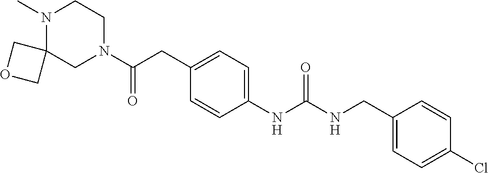 [SMILES: CN1CCN(C(=O)Cc2ccc(NC(=O)NCc3ccc(Cl)cc3)cc2)CC12COC2] |
| | | | Compound 73. {[(4-chlorophenyl)methyl]amino}- |
| | | | N-{4-[2-(9-methyl-2-oxa-6,9-diazaspiro[3.5]non-6- |
| | | | yl)-2-oxoethyl]phenyl}carboxamide. LCMS-ESI |
| | | | (POS.) m/z: 443.10 (M + H)+. ¹H NMR (400 MHz, |
| | | | DMSO-d₆) δ 8.55 (s, 1H), 7.40 (d, J = 8.0 Hz, 2H), |
| | | | 7.33 (d, J = 7.6 Hz, 4H), 7.13 (d, J = 8.1 Hz, 1H), |
| | | | 7.06 (d, J = 8.1 Hz, 1H), 6.63 (t, J = 6.1 Hz, 1H), |
| | | | 4.61 (dd, J = 16.7, 6.7 Hz, 2H), 4.28 (d, J = 5.9 Hz, |
| | | | 2H), 4.02 (dd, J = 29.3, 6.8 Hz, 2H), 3.69 (d, J = |
| | | | 10.5 Hz, 3H), 3.62 (s, 1H), 3.41 (s, 2H), 2.38 (t, J = |
| | | | 16.2 Hz, 4H), 2.25 (s, 1H). |
| 118 | 1.2 | (3R,4S)- pyrrolidine- 3,4-diol | 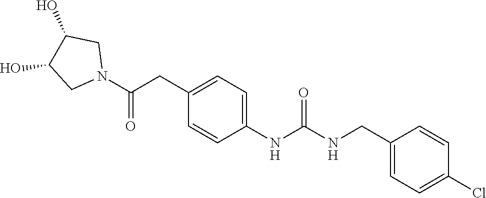 [SMILES: O=C(NCc1ccc(Cl)cc1)Nc1ccc(CC(=O)N2C[C@@H](O)[C@@H](O)C2)cc1] |
| | | | Compound 118. N-{4-[2-((4S,3R)-3,4- |
| | | | dihydroxypyrrolidinyl)-2-oxoethyl]phenyl}{[(4- |
| | | | chlorophenyl)methyl]amino}carboxamide. LCMS- |
| | | | ESI (POS.) m/z: 404.00 (M + H)+. ¹H NMR (400 |
| | | | MHz, DMSO-d₆) δ 8.54 (s, 1H), 7.40 (d, J = 8.0 Hz, |
| | | | 2H), 7.32 (d, J = 8.4 Hz, 4H), 7.08 (d, J = 8.1 Hz, |
| | | | 2H), 6.63 (t, J = 6.0 Hz, 1H), 4.96 (d, J = 5.3 Hz, |
| | | | 1H), 4.88 (d, J = 4.5 Hz, 1H), 4.28 (d, J = 6.0 Hz, |
| | | | 2H), 4.03 (q, J = 5.3 Hz, 1H), 3.98 (q, J = 4.6 Hz, |
| | | | 1H), 3.61 (dd, J = 10.2, 6.1 Hz, 1H), 3.48 (s, 2H), |
| | | | 3.38 (dd, J = 12.3, 5.4 Hz, 1H), 3.28 (dd, J = 10.2, |
| | | | 6.1 Hz, 1H), 3.18 (dd,J = 12.0, 4.5 Hz, 1H). |
| 121 | 1.2 | (3R,4R)- pyrrolidine- 3,4-diol | 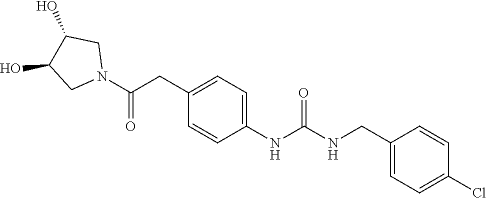 [SMILES: O=C(NCc1ccc(Cl)cc1)Nc1ccc(CC(=O)N2C[C@@H](O)[C@H](O)C2)cc1] |
| | | | Compound 121. N-{4-[2-((3R,4R)-3,4- |
| | | | dihydroxypyrrolidinyl)-2-oxoethyl]phenyl}{[(4- |
| | | | chlorophenyl)methyl]amino}carboxamide. LCMS- |
| | | | ESI (POS.) m/z: 404.00 (M + H)+. ¹H NMR (400 |
| | | | MHz, DMSO-d₆) δ 8.54 (s, 1H), 7.40 (d, J = 8.1 Hz, |
| | | | 2H), 7.32 (d, J = 8.3 Hz, 4H), 7.09 (d, J = 8.1 Hz, |
| | | | 2H), 6.63 (t, J = 6.1 Hz, 1H), 5.16 (d, J = 3.5 Hz, |
| | | | 1H), 5.08 (d, J = 3.2 Hz, 1H), 4.28 (d, J = 6.0 Hz, |
| | | | 2H), 3.96 (s, 1H), 3.89 (s, 1H), 3.62 (dd, J = 11.0, |
| | | | 4.1 Hz, 1H), 3.50 (s, 2H), 3.37 (d, J = 11.6 Hz, 2H), |
| | | | 3.28 (d, J = 12.5 Hz, 1H). |
| 280 | 1.1 | phenyl methanamine | 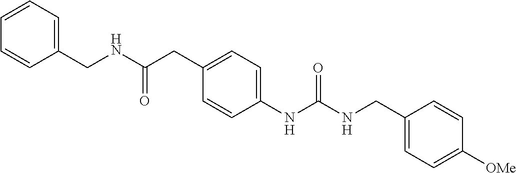 [SMILES: COc1ccc(CNC(=O)Nc2ccc(CC(=O)NCc3ccccc3)cc2)cc1] |
| | | | Compound 280. 2-[4-({N-[(4- |
| | | | methoxyphenyl)methyl]carbamoyl}amino)phenyl]- |
| | | | N-benzylacetamide. LCMS-ESI (POS.) m/z: |
| | | | 404.15 (M + H)+. ¹H NMR (400 MHz, DMSO-d₆) δ |
| | | | 8.44 (s, 1H), 7.32 (d, J = 8.0 Hz, 4H), 7.23 (d, J = 7.9 |
| | | | Hz, 3H), 7.10 (dd, J = 21.8, 8.2 Hz, 2H), 6.90 (d, J = |
| | | | 8.2 Hz, 2H), 6.49 (s, 1H), 4.24 (dd, J = 18.5, 5.9 Hz, |
| | | | 4H), 3.74 (s, 3H), 1.25 (s, 1H), 1.14-0.96 (m, 4H). |
| 289 | 1.1 | pyridin-2- ylmetha- namine | 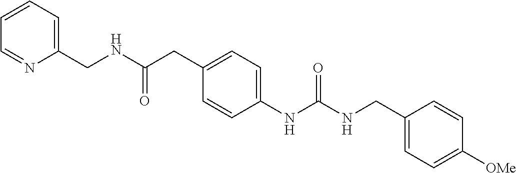 [SMILES: COc1ccc(CNC(=O)Nc2ccc(CC(=O)NCc3ccccn3)cc2)cc1] |
| | | | Compound 289. 2-[4-({N-[(4- |
| | | | methoxyphenyl)methyl]carbamoyl}amino)phenyl]- |
| | | | N-(2-pyridylmethyl)acetamide. LCMS-ESI (POS.) |
| | | | m/z: 405.10 (M + H)+. ¹H NMR (400 MHz, DMSO- |
| | | | d₆) δ 8.55 (s, 1H), 8.50 (s, 1H), 8.45 (s, 1H), 7.74 (t, |
| | | | J = 7.7 Hz, 1H), 7.32 (d, J = 8.0 Hz, 2H), 7.23 (d, J = |
| | | | 8.3 Hz, 4H), 7.15 (d, J = 8.0 Hz, 2H), 6.90 (d, J = 7.9 |
| | | | Hz, 2H), 6.48 (d, J = 6.1 Hz, 1H), 4.35 (d, J = 5.9 |
| | | | Hz, 2H), 4.22 (d, J = 5.7 Hz, 2H), 3.74 (s, 3H), 3.42 (s, 2H). |
| 311 | 1.1 | pyridin-3- ylmetha- namine | 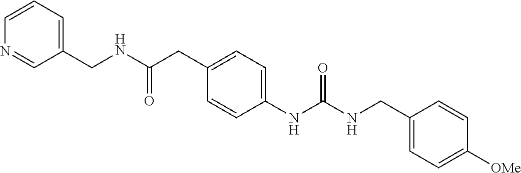 [SMILES: COc1ccc(CNC(=O)Nc2ccc(CC(=O)NCc3cccnc3)cc2)cc1] |
| | | | Compound 311. 2-[4-({N-[(4- |
| | | | methoxyphenyl)methyl]carbamoyl}amino)phenyl]- |
| | | | N-(3-pyridylmethyl)acetamide. LCMS-ESI (POS.) |
| | | | m/z: 405.10 (M + H)+. ¹H NMR (400 MHz, DMSO- |
| | | | d₆) δ 8.53 (s, 1H), 8.45 (d, J = 12.7 Hz, 2H), 7.70- |
| | | | 7.61 (m, 1H), 7.38 (dd, J = 7.5, 5.1 Hz, 1H), 7.32 (d, |
| | | | J = 8.1 Hz, 2H), 7.23 (d, J = 8.1 Hz, 2H), 7.12 (d, J = |
| | | | 8.0 Hz, 2H), 6.90 (d, J = 8.1 Hz, 2H), 6.48 (s, 1H), |
| | | | 4.30 (d, J = 5.9 Hz, 2H), 4.22 (d, J = 5.8 Hz, 2H), |
| | | | 3.74 (s, 3H), 3.39 (s, 3H). |
| 312 | 1.1 | pyridin-4- ylmetha- namine | 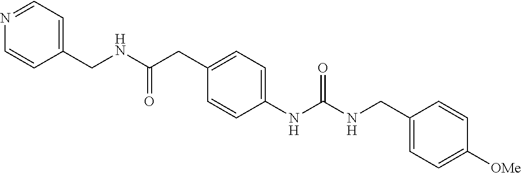 [SMILES: COc1ccc(CNC(=O)Nc2ccc(CC(=O)NCc3ccncc3)cc2)cc1] |
| | | | Compound 312. 2-[4-({N-[(4- |
| | | | methoxyphenyl)methyl]carbamoyl}amino)phenyl]- |
| | | | N-(4-pyridylmethyl)acetamide. LCMS-ESI (POS.) |
| | | | m/z: 405.10 (M + H)+. ¹H NMR (400 MHz, DMSO- |
| | | | d₆) δ 8.55 (s, 1H), 8.48 (d, J = 4.9 Hz, 2H), 8.45 (s, |
| | | | 1H), 7.33 (d, J = 8.0 Hz, 2H), 7.22 (t, J = 8.0 Hz, |
| | | | 4H), 7.14 (d, J = 8.1 Hz, 2H), 6.90 (d, J = 8.0 Hz, |
| | | | 2H), 6.49 (s, 1H), 4.29 (d, J = 5.9 Hz, 2H), 4.22 (d, J = |
| | | | 5.8 Hz, 2H), 3.74 (d, J = 1.6 Hz, 3H), 3.42 (s, 2H). |
| 299 | 1.1 | pyridazin-4- ylmetha- namine | 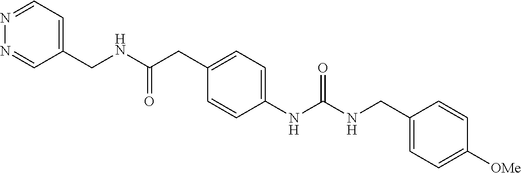 [SMILES: COc1ccc(CNC(=O)Nc2ccc(CC(=O)NCc3ccnnc3)cc2)cc1] |
| | | | Compound 299. 2-[4-({N-[(4- |
| | | | methoxyphenyl)methyl]carbamoyl} amino)phenyl]- |
| | | | N-(pyridazin-4-ylmethyl)acetamide. LCMS-ESI |
| | | | (POS.) m/z: 406.10 (M + H)+. ¹H NMR (400 MHz, |
| | | | DMSO-d₆) δ 9.13 (d, J = 5.4 Hz, 1H), 9.08 (s, 1H), |
| | | | 8.61 (d, J = 6.0 Hz, 1H), 8.48-8.41 (m, 1H), 7.46 |
| | | | (s, 1H), 7.32 (t, J = 8.8 Hz, 2H), 7.23 (d, J = 7.9 Hz, |
| | | | 2H), 7.14 (d, J = 8.4 Hz, 2H), 6.90 (d, J = 8.1 Hz, |
| | | | 1H), 6.49 (s, 2H), 4.33 (d, J = 6.0 Hz, 2H), 4.22 (d, |
| | | | J = 5.7 Hz, 3H), 3.74 (s, 4H). |
| 172 | 1.3 | 3-azabicyclo [3.1.0] hexan-1-ol | 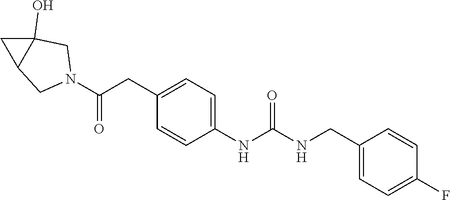 [SMILES: O=C(NCc1ccc(F)cc1)Nc1ccc(CC(=O)N2CC3CC3(O)C2)cc1] |
| | | | Compound 172. N-[(4-fluorophenyl)methyl]({4-[2- |
| | | | (1-hydroxy-3-azabicyclo[3.1.0]hex-3-yl)-2- |
| | | | oxoethyl]phenyl}amino)carboxamide. LCMS-ESI |
| | | | (POS.) m/z: 384.15 (M + H)+. ¹H NMR (400 MHz, |
| | | | DMSO-d₆) δ 8.51 (s, 1H), 7.34 (dd, J = 11.4, 4.5 Hz, |
| | | | 4H), 7.16 (t, J = 8.5 Hz, 2H), 7.05 (t, J = 8.3 Hz, |
| | | | 2H), 6.60 (s, 1H), 5.98-5.87 (m, 1H), 4.27 (d, J = |
| | | | 5.9 Hz, 2H), 3.81 (t, J = 9.4 Hz, 1H), 3.60 (s, 1H), |
| | | | 3.53-3.37 (m, 4H), 3.27-3.13 (m, 3H), 1.48 (d, J = |
| | | | 21.8 Hz, 1H), 0.98 (s, 1H), 0.32 (s, 1H). |
| 283 | 1.1 | 3-azabicyclo [3.1.0] hexan-1-ol | 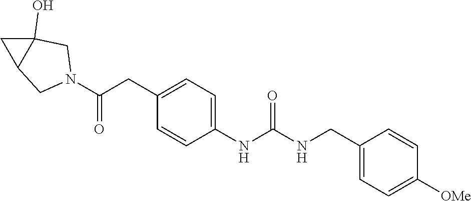 [SMILES: COc1ccc(CNC(=O)Nc2ccc(CC(=O)N3CC4CC4(O)C3)cc2)cc1] |
| | | | Compound 283. ({4-[2-(1-hydroxy-3- |
| | | | azabicyclo[3.1.0]hex-3-yl)-2-oxoethyl]phenyl}amino)-N-[(4- |
| | | | methoxyphenyl)methyl]carboxamide. LCMS-ESI |
| | | | (POS.) m/z: 369.10 (M + H)+. ¹H NMR (400 MHz, |
| | | | DMSO-d₆) δ 8.44 (s, 1H), 7.96 (s, 1H), 7.31 (s, 2H), |
| | | | 7.23 (d, J = 8.0 Hz, 2H), 7.04 (t, J = 8.1 Hz, 2H), |
| | | | 6.95-6.86 (m, 2H), 6.48 (s, 1H), 5.92 (d, J = 17.7 |
| | | | Hz, 1H), 4.22 (d, J = 5.8 Hz, 2H), 4.15-4.04 (m, |
| | | | 1H), 3.81 (t, J = 9.3 Hz, 1H), 3.74 (d, J = 1.7 Hz, |
| | | | 3H), 3.60 (s, 1H), 3.45 (dd, J = 21.7, 12.7 Hz, 3H), |
| | | | 3.18 (d, J = 5.4 Hz, 2H), 2.69 (d, J = 8.1 Hz, 1H), |
| | | | 1.48 (d, J = 22.6 Hz, 1H), 0.97 (s, OH), 0.33 (s, 1H). |
| 171 | 1.3 | ((1R,5S,6r)- 3-azabicyclo [3.1.0] hexan-6-yl) methanol | 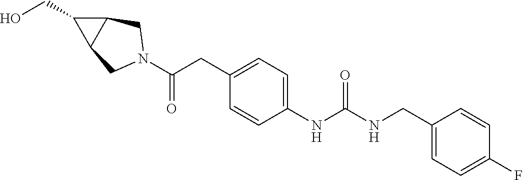 [SMILES: O=C(NCc1ccc(F)cc1)Nc1ccc(CC(=O)N2C[C@@H]3[C@@H](CO)[C@@H]3C2)cc1] |
| | | | Compound 171. [(4-{2-[(5S,1R)-6- |
| | | | (hydroxymethyl)-3-azabicyclo[3.1.0]hex-3-yl]-2- |
| | | | oxoethyl}phenyl)amino]-N-[(4- |
| | | | fluorophenyl)methyl]carboxamide. LCMS-ESI |
| | | | (POS.) m/z: 398.10 (M + H)+. ¹H NMR (400 MHz, |
| | | | DMSO-d₆) δ 8.50 (s, 1H), 7.39-7.28 (m, 4H), 7.16 |
| | | | (t, J = 8.4 Hz, 2H), 7.05 (d, J = 8.0 Hz, 2H), 6.60 (d, |
| | | | J = 6.4 Hz, 1H), 4.51 (s, 1H), 4.27 (d, J = 5.7 Hz, |
| | | | 2H), 3.60 (dd, J = 17.4, 11.0 Hz, 2H), 3.49 (d, J = |
| | | | 15.9 Hz, 3H), 3.31-3.22 (m, 3H), 1.46 (d,J = 22.9 |
| | | | Hz, 2H), 0.64 (s, 1H). |
| 249 | 1.1 | ((1R,5S,6r)- 3-azabicyclo [3.1.0] hexan-6-yl) methanol | 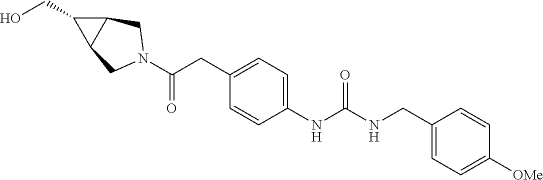 [SMILES: COc1ccc(CNC(=O)Nc2ccc(CC(=O)N3C[C@@H]4[C@@H](CO)[C@@H]4C3)cc2)cc1] |
| | | | Compound 249. [(4-{2-[(5S,1R)-6-(hydroxymethyl)-3- |
| | | | azabicyclo[3.1.0]hex-3-yl]-2-oxoethyl}phenyl)amino]-N-[(4- |
| | | | methoxyphenyl)methyl]carboxamide. LCMS-ESI |
| | | | (POS.) m/z: 410.10 (M + H)+. ¹H NMR (400 MHz, |
| | | | DMSO-d₆) δ 8.44 (s, 1H), 7.31 (d, J = 8.0 Hz, 2H), |
| | | | 7.23 (d, J = 8.0 Hz, 2H), 7.04 (d, J = 8.1 Hz, 2H), |
| | | | 6.90 (d, J = 8.1 Hz, 2H), 6.49 (t, J = 6.0 Hz, 1H), |
| | | | 4.50 (t, J = 5.6 Hz, 1H), 4.22 (d, J = 5.7 Hz, 2H), |
| | | | 3.74 (s, 3H), 3.60 (dd, J = 17.4, 11.1 Hz, 2H), 3.49 |
| | | | (d, J = 15.9 Hz, 3H), 3.31-3.23 (m, 3H), 1.46 (d, |
| | | | J = 22.3 Hz, 2H), 0.64 (s, 1H). |
| 126 | 1.2 | 1-methyl- 1,6- diazaspiro [3.3] heptane | 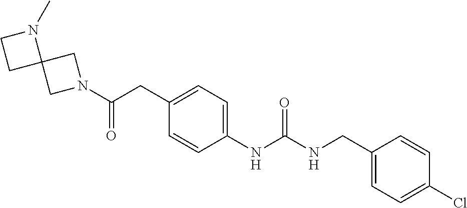 [SMILES: CN1CCC12CN(C(=O)Cc1ccc(NC(=O)NCc3ccc(Cl)cc3)cc1)C2] |
| | | | Compound 126. N-[(4-chlorophenyl)methyl]({4- |
| | | | [2-(5-methyl-2,5-diazaspiro[3.3]hept-2-yl)-2- |
| | | | oxoethyl]phenyl}amino)carboxamide. LCMS-ESI |
| | | | (POS.) m/z: 414.15 (M + H)+. ¹H NMR (400 MHz, |
| | | | DMSO-d₆) δ 8.54 (s, 1H), 8.14 (s, 1H), 7.48 (d, J = |
| | | | 7.7 Hz, 1H), 7.40 (d, J = 8.0 Hz, 2H), 7.33 (d, J = 7.6 |
| | | | Hz, 4H), 7.10 (dd, J = 17.8, 7.9 Hz, 3H), 6.63 (s, |
| | | | 1H), 4.49-4.39 (m, 2H), 4.28 (d, J = 5.7 Hz, 2H), |
| | | | 4.20-4.07 (m, 2H), 3.97-3.85 (m, 2H), 3.18 (d, J = |
| | | | 4.6 Hz, 1H), 2.68 (s, 1H), 2.32 (d, J = 16.8 Hz, 2H). |
| 48 | 1.2 | ((1R,5S,6r)- 3-azabicyclo [3.1.0]hexan- 6-yl) methanol | 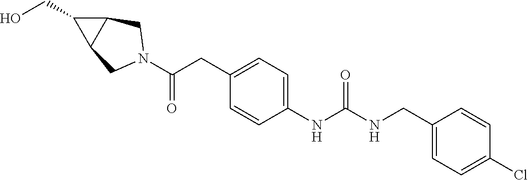 [SMILES: O=C(NCc1ccc(Cl)cc1)Nc1ccc(CC(=O)N2C[C@@H]3[C@@H](CO)[C@@H]3C2)cc1] |
| | | | Compound 48. [(4-{2-[(5S,1R)-6-(hydroxymethyl)-3-azabicyclo |
| | | | [3.1.0]hex-3-yl]-2-oxoethyl}phenyl)amino]-N-[(4- |
| | | | chlorophenyl)methyl]carboxamide. LCMS-ESI |
| | | | (POS.) m/z: 415.10 (M + H)+. ¹H NMR (400 MHz, |
| | | | DMSO-d₆) δ 8.54 (s, 1H), 7.40 (d, J = 7.8 Hz, 2H), |
| | | | 7.32 (d, J = 8.6 Hz, 3H), 7.05 (d, J = 8.1 Hz, 2H), |
| | | | 4.29 (s, 2H), 3.47 (s, 3H), 3.29 (d, J = 7.7 Hz, 8H), |
| | | | 3.18 (d, J = 5.4 Hz, 3H). |
| 342 | 1.1 | 1-methyl- 1,6- diazaspiro [3.3]heptane | 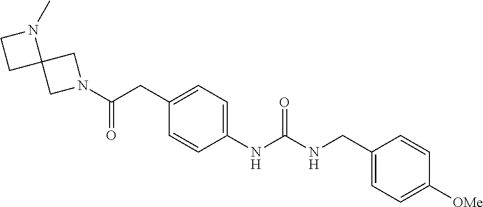 [SMILES: COc1ccc(CNC(=O)Nc2ccc(CC(=O)N3CC4(CCN4C)C3)cc2)cc1] |
| | | | Compound 342. N-[(4-methoxyphenyl)methyl]({4- |
| | | | [2-(5-methyl-2,5-diazaspiro[3.3]hept-2-yl)-2- |
| | | | oxoethyl]phenyl}amino)carboxamide. LCMS-ESI |
| | | | (POS.) m/z: 409.10 (M + H)+. ¹H NMR (400 MHz, |
| | | | DMSO-d₆) δ 8.45 (s, 1H), 8.14 (s, 1H), 7.48 (d, J = |
| | | | 7.8 Hz, 1H), 7.32 (d, J = 7.9 Hz, 2H), 7.23 (d, J = 8.2 Hz, |
| | | | 2H), 7.09 (dd, J = 18.8, 7.9 Hz, 3H), 6.90 (d, J = 8.1 Hz, 2H), |
| | | | 6.49 (s, 1H), 4.21 (d, J = 5.7 Hz, 3H), 3.74 (s, 3H), |
| | | | 3.18 (s, 1H), 2.46 (s, 6H), 2.32 (d, J = 16.9 Hz, 2H). |
| 53 | 1.2 | 3-azabicyclo [3.1.0] hexan-1-ol | 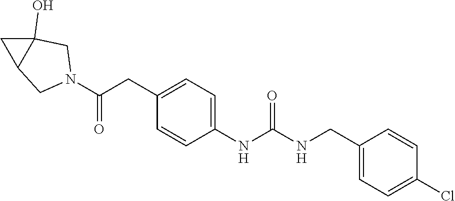 [SMILES: O=C(NCc1ccc(Cl)cc1)Nc1ccc(CC(=O)N2CC3CC3(O)C2)cc1] |
| | | | Compound 53. N-[(4-chlorophenyl)methyl]({4-[2- |
| | | | (1-hydroxy-3-azabicyclo[3.1.0]hex-3-yl)-2- |
| | | | oxoethyl]phenyl}amino)carboxamide. LCMS-ESI |
| | | | (POS.) m/z: 401.10 (M + H)+. ¹H NMR (400 MHz, |
| | | | DMSO-d₆) δ 8.54 (s, 1H), 7.40 (d, J = 8.0 Hz, 2H), |
| | | | 7.33 (d, J = 7.8 Hz, 4H), 7.05 (t, J = 8.1 Hz, 2H), |
| | | | 6.63 (s, 1H), 5.92 (d, J = 17.6 Hz, 1H), 4.28 (d, J = |
| | | | 5.9 Hz, 2H), 3.81 (t, J = 9.3 Hz, 1H), 3.61 (d, J = |
| | | | 11.1 Hz, 1H), 3.45 (dd, J = 22.3, 13.3 Hz, 5H), 3.20 |
| | | | (dd, J = 20.2, 8.2 Hz, 1H), 1.48 (d, J = 22.3 Hz, 2H), |
| | | | 1.05-0.92 (m, 1H), 0.32 (s, 1H). |
| 177 | 1.3 | ((1R,5S,6s)- 3-azabicyclo [3.1.0]hexan- 6-yl) methanol | 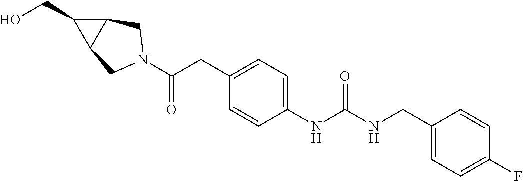 [SMILES: O=C(NCc1ccc(F)cc1)Nc1ccc(CC(=O)N2C[C@@H]3[C@H](CO)[C@@H]3C2)cc1] |
| | | | Compound 177. [(4-{2-[(5S,1R)-6- |
| | | | (hydroxymethyl)-3-azabicyclo[3.1.0]hex-3-yl]-2- |
| | | | oxoethyl}phenyl)amino]-N-[(4- |
| | | | fluorophenyl)methyl]carboxamide. LCMS-ESI |
| | | | (POS.) m/z: 398.10 (M + H)+. ¹H NMR (400 MHz, |
| | | | DMSO-d₆) δ 8.51 (s, 1H), 7.33 (dd, J = 7.5, 5.2 Hz, |
| | | | 4H), 7.16 (t, J = 8.3 Hz, 2H), 7.06 (d, J = 8.0 Hz, |
| | | | 2H), 6.64-6.54 (m, 1H), 4.43 (t, J = 5.5 Hz, 1H), |
| | | | 4.27 (d, J = 5.8 Hz, 2H), 4.10 (d, J = 5.2 Hz, 1H), |
| | | | 3.64 (dd, J = 10.9, 5.3 Hz, 1H), 3.54 (d, J = 10.6 Hz, |
| | | | 1H), 3.45 (d, J = 8.6 Hz, 3H), 3.29-3.16 (m, 2H), |
| | | | 1.81-1.57 (m, 2H), 1.09 (p, J = 7.7 Hz, 1H). |
| 266 | 1.1 | ((1R,5S,6r)- 3-azabicyclo [3.1.0]hexan- 6-yl) methanol | 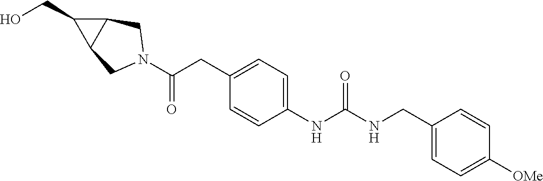 [SMILES: COc1ccc(CNC(=O)Nc2ccc(CC(=O)N3C[C@@H]4[C@H](CO)[C@@H]4C3)cc2)cc1] |
| | | | Compound 266: [(4-{2-[(5S,1R)-6- |
| | | | (hydroxymethyl)-3-azabicyclo[3.1.0]hex-3-yl]-2- |
| | | | oxoethyl}phenyl)amino]-N-[(4- |
| | | | methoxyphenyl)methyl]carboxamide. LCMS-ESI |
| | | | (POS.) m/z: 415.1 (M + H)+. 1H NMR (400 MHz, |
| | | | DMSO-d6) δ 8.54 (s, 1H), 7.40 (d, J = 8.0 Hz, 2H), |
| | | | 7.32 (d, J = 8.4 Hz, 4H), 7.06 (d, J = 8.1 Hz, 2H), |
| | | | 6.63 (t, J = 5.9 Hz, 1H), 4.43 (t, J = 5.3 Hz, 1H), 4.28 |
| | | | (d, J = 5.9 Hz, 2H), 4.10 (d, J = 5.6 Hz, 1H), 3.64 |
| | | | (dd, J = 10.6, 5.3 Hz, 1H), 3.54 (d, J = 10.7 Hz, 1H), |
| | | | 3.45 (d, J = 9.0 Hz, 3H), 3.18 (d, J = 5.2 Hz, 2H), |
| | | | 1.81-1.60 (m, 2H), 1.10 (q, J = 7.9 Hz, 1H). |
| 61 | 1.2 | ((1R,5S,6r)- 3-azabicyclo [3.1.0]hexan- 6-yl) methanol | 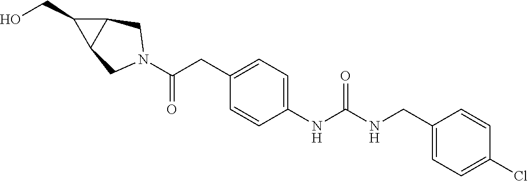 [SMILES: O=C(NCc1ccc(Cl)cc1)Nc1ccc(CC(=O)N2C[C@@H]3[C@H](CO)[C@@H]3C2)cc1] |
| | | | Compound 61: [(4-{2-[(5S,1R)-6- |
| | | | (hydroxymethyl)-3-azabicyclo[3.1.0]hex-3-yl]-2- |
| | | | oxoethyl}phenyl)amino]-N-[(4- |
| | | | chlorophenyl)methyl]carboxamide. LCMS-ESI |
| | | | (POS.) m/z: 415.1 (M + H)+. 1H NMR (400 MHz, |
| | | | DMSO-d6) δ 8.54 (s, 1H), 7.40 (d, J = 8.0 Hz, 2H), |
| | | | 7.32 (d, J = 8.4 Hz, 4H), 7.06 (d, J = 8.1 Hz, 2H), |
| | | | 6.63 (t, J = 5.9 Hz, 1H), 4.43 (t, J = 5.3 Hz, 1H), 4.28 |
| | | | (d, J = 5.9 Hz, 2H), 4.10 (d, J = 5.6 Hz, 1H), 3.64 |
| | | | (dd, J = 10.6, 5.3 Hz, 1H), 3.54 (d, J = 10.7 Hz, 1H), |
| | | | 3.45 (d, J = 9.0 Hz, 3H), 3.18 (d, J = 5.2 Hz, 2H), |
| | | | 1.81-1.60 (m, 2H), 1.10 (q, J = 7.9 Hz, 1H). |
| 193 | 1.3 | pyrazin-2- ylmetha- namine | 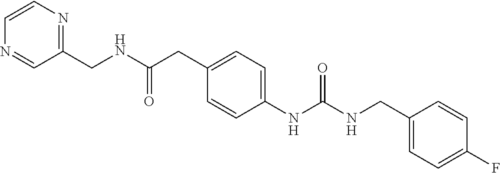 [SMILES: O=C(Cc1ccc(NC(=O)NCc2ccc(F)cc2)cc1)NCc1cnccn1] |
| | | | Compound 193. 2-[4-({N-[(4- |
| | | | fluorophenyl)methyl]carbamoyl}amino)phenyl]- |
| | | | N-(pyrazin-2-ylmethyl)acetamide. LCMS-ESI |
| | | | (POS.) m/z: 394.10 (M + H)+. ¹H NMR (400 MHz, |
| | | | DMSO-d₆) δ 8.64 (s, 1H), 8.58 (s, 1H), 8.51 (d, J = |
| | | | 10.9 Hz, 3H), 7.33 (d, J = 7.7 Hz, 4H), 7.15 (t, J = |
| | | | 6.1 Hz, 4H), 6.61 (s, 1H), 4.41 (d, J = 5.8 Hz, 2H), |
| | | | 4.28 (d, J = 5.9 Hz, 2H), 3.42 (s, 2H). |
| 83 | 1.2 | pyrazin-2- ylmetha- namine | 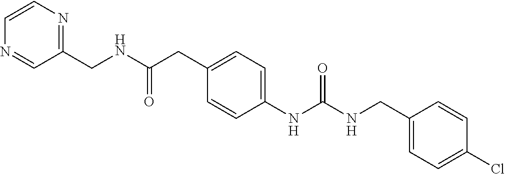 [SMILES: O=C(Cc1ccc(NC(=O)NCc2ccc(Cl)cc2)cc1)NCc1cnccn1] |
| | | | Compound 83. 2-[4-({N-[(4- |
| | | | chlorophenyl)methyl]carbamoyl}amino)phenyl]- |
| | | | N-(pyrazin-2-ylmethyl)acetamide. LCMS-ESI |
| | | | (POS.) m/z: 411.10 (M + H)+. ¹H NMR (400 MHz, |
| | | | DMSO-d₆) δ 8.64 (s, 1H), 8.54 (dd,J = 20.8, 12.1 |
| | | | Hz, 4H), 7.40 (d, J = 8.2 Hz, 2H), 7.33 (d, J = 7.7 |
| | | | Hz, 4H), 7.14 (d, J = 8.0 Hz, 2H), 6.63 (s, 1H), 4.41 |
| | | | (d, J = 5.9 Hz, 2H), 4.28 (d, J = 6.1 Hz, 2H), 3.42 (s, 2H). |
| 321 | 1.1 | pyrazin-2- ylmetha- namine | 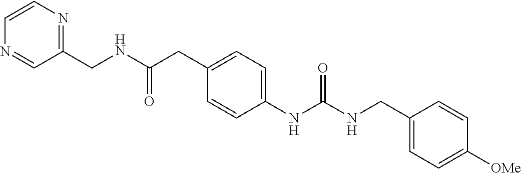 [SMILES: COc1ccc(CNC(=O)Nc2ccc(CC(=O)NCc3cnccn3)cc2)cc1] |
| | | | Compound 321. 2-[4-({N-[(4- |
| | | | methoxyphenyl)methyl]carbamoyl}amino)phenyl]- |
| | | | N-(pyrazin-2-ylmethyl)acetamide. LCMS-ESI |
| | | | (POS.) m/z: 406.10 (M + H)+. ¹H NMR (400 MHz, |
| | | | DMSO-d₆) δ 8.64 (s, 1H), 8.52 (dd, J = 30.8, 20.6 |
| | | | Hz, 4H), 7.32 (d, J = 8.0 Hz, 2H), 7.23 (d, J = 8.4 |
| | | | Hz, 2H), 7.14 (d, J = 8.2 Hz, 2H), 6.90 (d, J = 8.2 |
| | | | Hz, 2H), 6.49 (s, 1H), 4.41 (d, J = 6.0 Hz, 2H), 4.22 |
| | | | (d, J = 5.9 Hz, 2H), 4.10 (d, J = 5.4 Hz, 2H), 3.74 (s, 3H). |
| 352 | 1.1 | tert-butyl 1,6- diazaspiro [3.3] heptane-6- carboxylate | 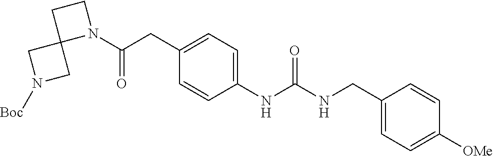 [SMILES: COc1ccc(CNC(=O)Nc2ccc(CC(=O)N3CCC34CN(C(=O)OC(C)(C)C)C4)cc2)cc1] |
| | | | Compound 352. tert-butyl 5-{2-[4-({[(4- |
| | | | methoxyphenyl)methyl]amino}carbonylamino) |
| | | | phenyl]acetyl}-2,5-diazaspiro[3.3]heptane-2- |
| | | | carboxylate. LCMS-ESI (POS.) m/z: 395.15 |
| | | | (M + H)+. ¹H NMR (400 MHz, DMSO-d₆) δ 8.50 (t, |
| | | | J = 6.9 Hz, 1H), 7.33 (s, 2H), 7.23 (d, J = 7.8 Hz, 2H), |
| | | | 7.10 (t, J = 10.7 Hz, 2H), 6.90 (d, J = 7.6 Hz, 2H), |
| | | | 6.55 (s, 1H), 4.58 (s, 3H), 4.47 (s, 3H), 4.22 (d, J = |
| | | | 5.2 Hz, 2H), 4.09 (d, J = 14.7 Hz, 3H), 3.95 (d, J = |
| | | | 20.8 Hz, 4H), 3.73 (s, 3H), 1.37 (s, 9H). |
| 215 | 1.1 | 2,2- dimethylaze- tidine | 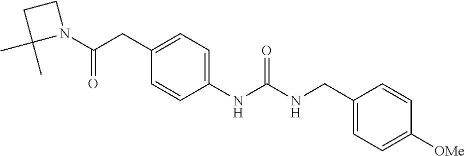 [SMILES: COc1ccc(CNC(=O)Nc2ccc(CC(=O)N3CCC3(C)C)cc2)cc1] |
| | | | Compound 215. ({4-[2-(2,2-dimethylazetidinyl)-2- |
| | | | oxoethyl]phenyl}amino)-N-[(4- |
| | | | methoxyphenyl)methyl]carboxamide. LCMS-ESI |
| | | | (POS.) m/z: 382.20 (M + H)+. ¹H NMR (400 MHz, |
| | | | DMSO-d₆) δ 8.47 (d, J = 5.2 Hz, 1H), 7.31 (t, J = 6.9 |
| | | | Hz, 2H), 7.23 (d, J = 8.1 Hz, 2H), 7.08 (t, J = 9.3 Hz, |
| | | | 2H), 6.93-6.87 (m, 2H), 6.53 (t, J = 5.8 Hz, 1H), |
| | | | 4.22 (d, J = 5.8 Hz, 2H), 3.98 (t, J = 7.7 Hz, 2H), |
| | | | 3.74 (s, 3H), 1.99 (dt, J = 24.2, 7.8 Hz, 2H), 1.51 (s, |
| | | | 2H), 1.42 (s, 6H). |
| 170 | 1.3 | 2,2- dimethylaze- tidine | 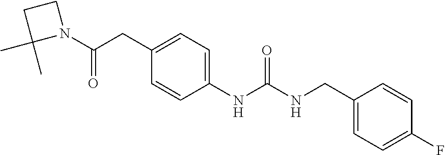 [SMILES: CC1(C)CCN1C(=O)Cc1ccc(NC(=O)NCc2ccc(F)cc2)cc1] |
| | | | Compound 170. ({4-[2-(2,2-dimethylazetidinyl)-2- |
| | | | oxoethyl]phenyl}amino)-N-[(4- |
| | | | fluorophenyl)methyl]carboxamide. LCMS-ESI |
| | | | (POS.) m/z: 370.10 (M + H)+. ¹H NMR (400 MHz, |
| | | | DMSO-d₆) δ 8.51 (s, 1H), 7.42-7.27 (m, 4H), 7.16 |
| | | | (t, J = 8.6 Hz, 2H), 7.09 (d, J = 7.7 Hz, 2H), 6.61 (s, |
| | | | 1H), 4.27 (d, J = 6.0 Hz, 3H), 3.30-3.20 (m, 3H), |
| | | | 3.16-3.05 (m, 1H), 1.61-1.36 (m, 3H), 1.16 (s, |
| | | | 1H), 1.08 (s, 3H). |

### Example 3

### Synthesis of N-{4-[(cyclobutylcarbonylamino)methyl]phenyl}{[(4-methoxyphenyl)methyl]amino}carboxamide (Compound 221)

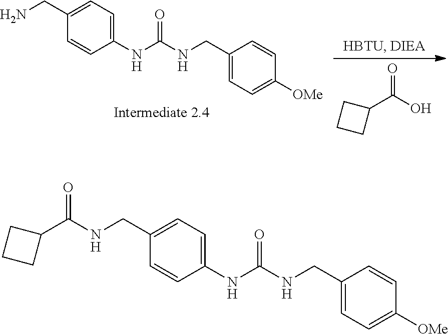 [SMILES: COc1ccc(CNC(=O)Nc2ccc(CN)cc2)cc1 ; O=C(O)C1CCC1 ; COc1ccc(CNC(=O)Nc2ccc(CNC(=O)C3CCC3)cc2)cc1]

A scintillation vial was charged with cyclobutanecarboxylic acid (42 mg, 0.63 mmol, 1.0 equiv) and O-(benzotriazol-1-yl)-N,N,N′,N′-tetramethyluronium hexafluorophosphate (320 mg, 0.84 mmol, 2.0 equiv) in dimethylformamide (2 mL). N, N-diisopropylethylamine was added (37 μL, 0.21 mmol, 0.5 equiv). 1-(4-(Aminomethyl)phenyl)-3-(4-methoxybenzyl)urea (180 mg, 0.63 mmol, 1.5 equiv) was added and the resulting mixture was stirred at room temperature for approximately 30 minutes. Resultant reaction mixture was diluted with water (5 mL) and extracted with ethyl acetate (2×8 mL). The organic phase was dried to a viscous oil which was purified by reverse phase HPLC with a 100-1000 acetonitrile in water solution that was run over 30 minutes in a Phenomonex Gemini 5u C18 column, providing the desired product (38.0 mg, 0.10 mmol, 25% yield) as a white solid. LCMS-APCI (POS.) m/z: 368.15 (M+H)+. 1H NMR (400 MHz, DMSO-d6) δ 8.45 (s, 1H), 7.33 (d, J=8.0 Hz, 2H), 7.23 (d, J=8.0 Hz, 2H), 7.08 (d, J=8.1 Hz, 2H), 6.90 (d, J=8.0 Hz, 2H), 6.48 (t, J=6.0 Hz, 1H), 4.18 (dd, J=22.4, 5.9 Hz, 4H), 3.74 (d, J=1.5 Hz, 3H), 3.04 (p, J=8.6 Hz, 1H), 2.14 (p, J=9.4 Hz, 2H), 2.02 (d, J=9.4 Hz, 2H), 1.88 (q, J=9.1 Hz, 1H), 1.76 (d, J=10.0 Hz, 2H).

Compounds in the following table were prepared in a similar manner as Compound 221, using the intermediates and reagents as listed.

| Ex | | Carboxylic | |
|---|---|---|---|
| # | Intermediate | Acid | Structure, Name and Data |
| 305 | 2.4 | oxetane-3- carboxylic acid | 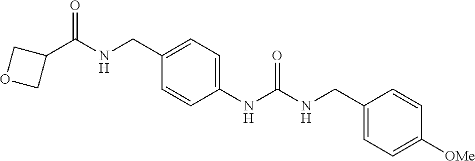 [SMILES: COc1ccc(CNC(=O)Nc2ccc(CNC(=O)C3COC3)cc2)cc1] |
| | | | Compound 305. {[(4- |
| | | | methoxyphenyl)methyl]amino}-N-{4-[(oxetan-3- |
| | | | ylcarbonylamino)methyl]phenyl}carboxamide. |
| | | | LCMS-ESI (POS.) m/z: 370.10 (M + H)+. ¹H NMR |
| | | | (400 MHz, DMSO-d₆) δ 8.46 (s, 1H), 8.30 (s, 1H), |
| | | | 7.34 (d, J = 8.0 Hz, 2H), 7.23 (d, J = 8.2 Hz, 2H), |
| | | | 7.11 (d, J = 8.3 Hz, 2H), 6.90 (d, J = 8.1 Hz, 2H), |
| | | | 6.48 (t, J = 5.8 Hz, 1H), 4.63 (d, J = 7.2 Hz, 4H), |
| | | | 4.21 (d, J = 5.7 Hz, 4H), 3.84-3.69 (m, 4H). |
| 234 | 2.4 | 3- methyloxetane- 3-carboxylic acid | 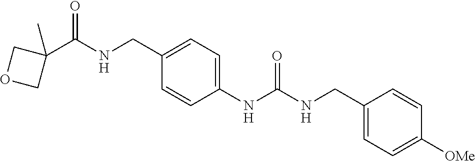 [SMILES: COc1ccc(CNC(=O)Nc2ccc(CNC(=O)C3(C)COC3)cc2)cc1] |
| | | | Compound 305. {[(4- |
| | | | methoxyphenyl)methyl]amino}-N-(4-{[(3- |
| | | | methyloxetan-3- |
| | | | yl)carbonylamino]methyl}phenyl)carboxamide. |
| | | | LCMS-ESI (POS.) m/z: 384.15 (M + H)+. |
| | | | ¹H NMR (400 MHz, DMSO-d₆) δ 8.46 (s, 1H), 8.32- |
| | | | 8.25 (m, 1H), 7.35 (d, J = 8.1 Hz, 2H), 7.23 (d, J = |
| | | | 8.1 Hz, 2H), 7.11 (d, J = 8.0 Hz, 2H), 6.94-6.86 |
| | | | (m, 2H), 6.49 (t, J = 6.0 Hz, 1H), 4.73 (d, J = 5.9 |
| | | | Hz, 2H), 4.24 (dd, J = 17.0, 5.8 Hz, 6H), 3.74 (d, J = |
| | | | 1.4 Hz, 3H), 1.51 (s, 3H). |
| 254 | 2.4 | cyclopentane- carboxylic acid | 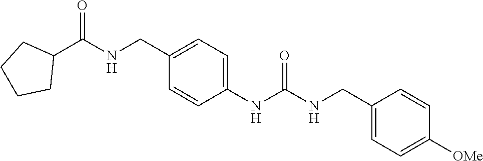 [SMILES: COc1ccc(CNC(=O)Nc2ccc(CNC(=O)C3CCCC3)cc2)cc1] |
| | | | Compound 254. N-{4- |
| | | | [(cyclopentylcarbonylamino)methyl]phenyl}{[(4- |
| | | | methoxyphenyl)methyl]amino}carboxamide. |
| | | | LCMS-ESI (POS.) m/z: 382.10 (M + H)+. ¹H NMR |
| | | | (400 MHz, DMSO-d₆) δ 8.45 (s, 1H), 8.18 (s, 1H), |
| | | | 7.33 (d, J = 8.0 Hz, 2H), 7.23 (d, J = 8.1 Hz, 2H), |
| | | | 7.09 (d, J = 8.1 Hz, 2H), 6.98-6.80 (m, 2H), 6.48 |
| | | | (t, J = 5.9 Hz, 1H), 4.19 (dd, J = 18.7, 5.9 Hz, 4H), |
| | | | 3.74 (d, J = 1.5 Hz, 3H), 2.59 (t, J = 7.7 Hz, 1H), |
| | | | 1.86-1.70 (m, 2H), 1.63 (s, 4H), 1.50 (s, 2H). |
| 292 | 2.4 | (S)- tetrahydrofuran- 2-carboxylic acid | 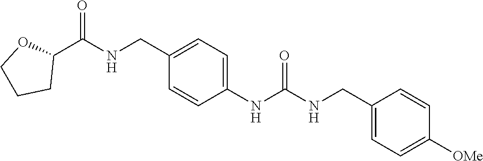 [SMILES: COc1ccc(CNC(=O)Nc2ccc(CNC(=O)[C@@H]3CCCO3)cc2)cc1] |
| | | | Compound 292. N-(4-{[((2S)oxolan-2- |
| | | | yl)carbonylamino]methyl}phenyl){[(4- |
| | | | methoxyphenyl)methyl]amino}carboxamide. |
| | | | LCMS-ESI (POS.) m/z: 384.10 (M + H)+. ¹H NMR |
| | | | (400 MHz, DMSO-d₆) δ 8.45 (s, 1H), 8.22 (t, J = |
| | | | 6.4 Hz, 1H), 7.32 (d, J = 8.1 Hz, 2H), 7.23 (d, J = |
| | | | 8.1 Hz, 2H), 7.09 (d, J = 8.1 Hz, 2H), 6.94-6.84 |
| | | | (m, 2H), 6.56-6.41 (m, 1H), 4.21 (td, J = 13.3, |
| | | | 12.6, 6.8 Hz, 5H), 3.90 (q, J = 6.9 Hz, 1H), 3.81- |
| | | | 3.72 (m, 4H), 2.21-2.05 (m, 1H), 1.84 (ddt, J = |
| | | | 23.7, 12.0, 6.8 Hz, 3H). |
| 319 | 2.4 | (R)- tetrahydrofuran- 2-carboxylic acid | 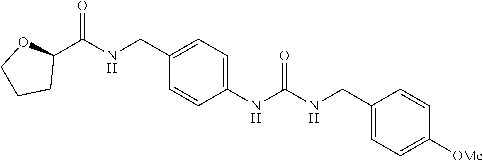 [SMILES: COc1ccc(CNC(=O)Nc2ccc(CNC(=O)[C@H]3CCCO3)cc2)cc1] |
| | | | Compound 319. N-(4-{[((2R)oxolan-2- |
| | | | yl)carbonylamino]methyl}phenyl){[(4- |
| | | | methoxyphenyl)methyl]amino}carboxamide. |
| | | | LCMS-ESI (POS.) m/z: 384.10 (M + H)+. ¹H NMR |
| | | | (400 MHz, DMSO-d₆) δ 8.45 (s, 1H), 8.22 (s, 1H), |
| | | | 7.32 (d, J = 8.1 Hz, 2H), 7.23 (d, J = 8.1 Hz, 2H), |
| | | | 7.09 (d, J = 8.0 Hz, 2H), 6.90 (d, J = 8.1 Hz, 2H), |
| | | | 6.48 (t, J = 6.0 Hz, 1H), 4.22 (td, J = 13.3, 12.6, 6.2 |
| | | | Hz, 5H), 3.90 (q, J = 6.9 Hz, 1H), 3.81-3.71 (m, |
| | | | 4H), 2.21- 2.06 (m, 1H), 1.84 (dp, J = 23.4, 7.2 Hz, |
| | | | 3H). |
| 303 | 2.4 | tetrahydrofuran- 3-carboxylic acid | 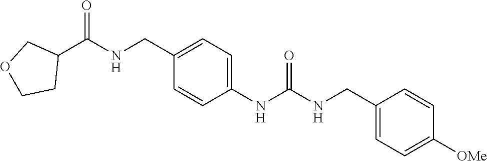 [SMILES: COc1ccc(CNC(=O)Nc2ccc(CNC(=O)C3CCOC3)cc2)cc1] |
| | | | Compound 303. {[(4- |
| | | | methoxyphenyl)methyl]amino}-N-{4-[(oxolan-3- |
| | | | ylcarbonylamino)methyl]phenyl}carboxamide. |
| | | | LCMS-ESI (POS.) m/z: 384.10 (M + H)+. ¹H NMR |
| | | | (400 MHz, DMSO-d₆) δ 8.46 (s, 1H), 8.36 (s, 1H), |
| | | | 7.34 (d, J = 8.1 Hz, 2H), 7.23 (d, J = 8.1 Hz, 2H), |
| | | | 7.10 (d, J = 8.1 Hz, 2H), 6.90 (d, J = 8.2 Hz, 2H), |
| | | | 6.49 (t, J = 6.1 Hz, 1H), 4.20 (dd, J = 11.7, 5.8 Hz, |
| | | | 4H), 3.86 (t, J = 8.1 Hz, 1H), 3.74 (s, 4H), 3.65 (dt, |
| | | | J = 16.2, 7.5 Hz, 2H), 2.97 (p, J = 7.6 Hz, 1H), 1.99 |
| | | | (q, J = 7.2 Hz, 2H). |
| 278 | 2.4 | cyclohexane carboxylic acid | 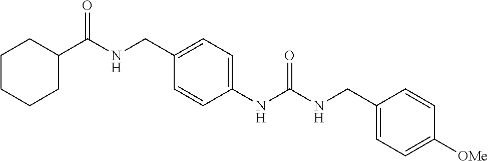 [SMILES: COc1ccc(CNC(=O)Nc2ccc(CNC(=O)C3CCCCC3)cc2)cc1] |
| | | | Compound 278. N-{4- |
| | | | [(cyclohexylcarbonylamino)methyllphenyl}{[(4- |
| | | | methoxyphenyl)methyl]amino}carboxamide. |
| | | | LCMS-ESI (POS.) m/z: 396.15 (M + H)+. ¹H NMR |
| | | | (400 MHz, DMSO-d₆) δ 8.44 (s, 1H), 8.11 (s, 1H), |
| | | | 7.32 (d, J = 8.1 Hz, 2H), 7.23 (d, J = 8.1 Hz, 2H), |
| | | | 7.08 (d, J = 8.1 Hz, 2H), 6.90 (d, J = 7.8 Hz, 2H), |
| | | | 6.48 (t, J = 5.8 Hz, 1H), 4.18 (dd, J = 24.3, 5.8 Hz, |
| | | | 4H), 3.74 (d, J = 1.6 Hz, 3H), 2.14 (t, J = 11.8 Hz, |
| | | | 1H), 1.76- 1.57 (m, 5H), 1.43-1.11 (m, 5H). |
| 274 | 2.4 | tetrahydro- 2H-pyran-3- carboxylic acid | 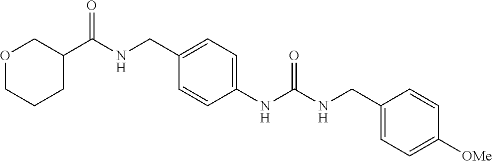 [SMILES: COc1ccc(CNC(=O)Nc2ccc(CNC(=O)C3CCCOC3)cc2)cc1] |
| | | | Compound 274. {[(4- |
| | | | methoxyphenyl)methyl]amino}-N-{4-[(2H- |
| | | | 3,4,5,6-tetrahydropyran-3- |
| | | | ylcarbonylamino)methyl]phenyl}carboxamide. |
| | | | LCMS-ESI (POS.) m/z: 398.10 (M + H)+. ¹H NMR |
| | | | (400 MHz, DMSO-d₆) δ 8.45 (s, 1H), 8.28 (s, 1H), |
| | | | 7.33 (d, J = 8.0 Hz, 2H), 7.23 (d, J = 8.1 Hz, 2H), |
| | | | 7.08 (d, J = 8.0 Hz, 2H), 6.90 (d, J = 8.1 Hz, 2H), |
| | | | 6.48 (t, J = 5.9 Hz, 1H), 4.19 (dd, J = 23.0, 5.8 Hz, |
| | | | 4H), 3.93-3.68 (m, 5H), 3.39-3.34 (m, 1H), 3.31- |
| | | | 3.24 (m, 1H), 1.92-1.78 (m, 1H), 1.74- 1.36 (m, |
| | | | 4H). |
| 250 | 2.4 | tetrahydro- 2H-pyran-4- carboxylic acid | 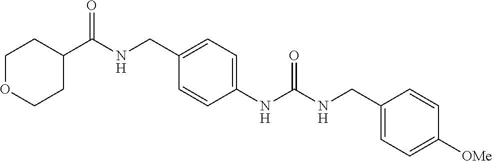 [SMILES: COc1ccc(CNC(=O)Nc2ccc(CNC(=O)C3CCOCC3)cc2)cc1] |
| | | | Compound 250. {[(4- |
| | | | methoxyphenyl)methyl]amino}-N-{4-[(2H- |
| | | | 3,4,5,6-tetrahydropyran-4- |
| | | | ylcarbonylamino)methyl]phenyl}carboxamide. |
| | | | LCMS-ESI (POS.) m/z: 398.10 (M + H)+. ¹H NMR |
| | | | (400 MHz, DMSO-d₆) δ 8.45 (s, 1H), 8.22 (d, J = |
| | | | 6.5 Hz, 1H), 7.33 (d, J = 8.0 Hz, 2H), 7.23 (d, J = |
| | | | 8.2 Hz, 2H), 7.09 (d, J = 8.1 Hz, 2H), 6.90 (d, J = |
| | | | 8.1 Hz, 2H), 6.54-6.43 (m, 1H), 4.19 (dd, J = 16.2, |
| | | | 5.8 Hz, 4H), 3.86 (d, J = 11.4 Hz, 2H), 3.74 (s, 3H), |
| | | | 3.32-3.25 (m, 2H), 2.40 (p, J = 8.5 Hz, 1H), 1.61 |
| | | | (q, J = 5.6 Hz, 4H). |
| 256 | 2.4 | 3- isopropylcyclo- butane-1- carboxylic acid | 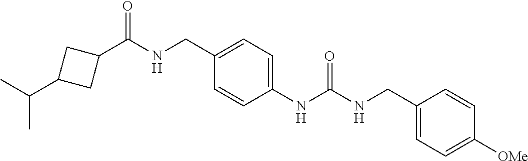 [SMILES: COc1ccc(CNC(=O)Nc2ccc(CNC(=O)C3CC(C(C)C)C3)cc2)cc1] |
| | | | Compound 256. {[(4- |
| | | | methoxyphenyl)methyl]amino}-N-[4-({[3- |
| | | | (methylethyl)cyclobutyl]carbonylamino}methyl) |
| | | | phenyl]carboxamide. LCMS-ESI (POS.) m/z: |
| | | | 410.10 (M + H)+. ¹H NMR (400 MHz, DMSO-d₆) δ |
| | | | 8.44 (s, 1H), 8.15-7.99 (m, 1H), 7.33 (d, J = 8.1 |
| | | | Hz, 2H), 7.23 (d, J = 8.1 Hz, 2H), 7.08 (dd, J = 8.4, |
| | | | 4.5 Hz, 2H), 6.98-6.79 (m, 2H), 6.48 (t, J = 5.8 |
| | | | Hz, 1H),4.21 (d, J = 5.7Hz, 2H), 4.16 (dd,J = 10.1, |
| | | | 6.1 Hz, 2H), 3.74 (d, J = 1.5 Hz, 3H), 2.94-2.72 |
| | | | (m, 1H), 2.22-2.01 (m, 2H), 1.96 (q, J = 8.0 Hz, |
| | | | 1H), 1.79 (dt, J = 28.7, 8.8 Hz, 3H), 1.56- 1.33 (m, |
| | | | 1H), 0.78 (ddd, J = 11.0, 6.6, 1.6 Hz, 5H). |
| 262 | 2.4 | cyclopropane- carboxylic acid | 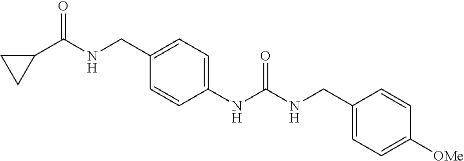 [SMILES: COc1ccc(CNC(=O)Nc2ccc(CNC(=O)C3CC3)cc2)cc1] |
| | | | Compound 262. N-{4- |
| | | | [(cyclopropylcarbonylamino)methyl]phenyl} |
| | | | {[(4-methoxyphenyl)methyl]amino}carboxamide. |
| | | | LCMS-ESI (POS.) m/z: 354.10 (M + H)+. ¹H NMR |
| | | | (400 MHz, DMSO-d₆) δ 8.46 (d, J = 5.8 Hz, 2H), |
| | | | 7.34 (d, J = 8.0 Hz, 2H), 7.23 (d, J = 8.1 Hz, 2H), |
| | | | 7.11 (d, J = 8.0 Hz, 2H), 6.90 (d, J = 8.1 Hz, 2H), |
| | | | 6.49 (t, J = 5.5 Hz, 1H), 4.20 (dd, J = 11.6, 5.8 Hz, |
| | | | 4H), 3.74 (s, 3H), 1.67-1.52 (m, 1H), 0.75-0.58 |
| | | | (m, 4H). |
| 227 | 2.4 | 1- methylcyclo- propane-1- carboxylic acid | 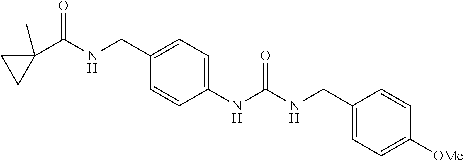 [SMILES: COc1ccc(CNC(=O)Nc2ccc(CNC(=O)C3(C)CC3)cc2)cc1] |
| | | | Compound 227. {[(4- |
| | | | methoxyphenyl)methyl]amino}-N-(4- |
| | | | {[(methyl cyclopropyl)carbonylamino]methyl} |
| | | | phenyl)carboxamide. LCMS-ESI (POS.) m/z: |
| | | | 368.10 (M + H)+. ¹H NMR (400 MHz, DMSO-d₆) δ |
| | | | 8.44 (s, 1H), 7.99 (t, J = 6.1 Hz, 1H), 7.32 (d, J = |
| | | | 8.1 Hz, 2H), 7.23 (d, J = 8.1 Hz, 2H), 7.09 (d, J = |
| | | | 8.1 Hz, 2H), 6.90 (d, J = 7.9 Hz, 2H), 6.48 (t, J = |
| | | | 5.9 Hz, 1H), 4.20 (dd, J = 13.0, 5.9 Hz, 4H), 3.74 |
| | | | (d, J = 1.6 Hz, 3H), 1.28 (s, 3H), 0.96 (d, J = 3.0 |
| | | | Hz, 2H), 0.51 (d, J = 2.9 Hz, 2H). |
| 268 | 2.4 | 2- cyclopentyl acetic acid | 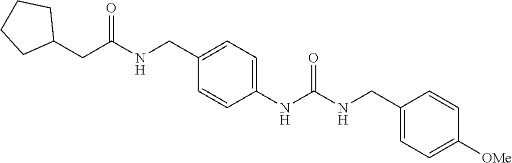 [SMILES: COc1ccc(CNC(=O)Nc2ccc(CNC(=O)CC3CCCC3)cc2)cc1] |
| | | | Compound 268.2-cyclopentyl-N-{[4-({[(4- |
| | | | methoxyphenyl)methyl]amino}carbonylamino) |
| | | | phenyl]methyl}acetamide. LCMS-ESI (POS.) |
| | | | m/z: 396.10 (M + H)+. ¹H NMR (400 MHz, DMSO- |
| | | | d₆) δ 8.44 (s, 1H), 8.19 (s, 1H), 7.33 (d, J = 8.1 Hz, |
| | | | 2H), 7.23 (d, J = 8.1 Hz, 2H), 7.10 (d, J = 8.1 Hz, |
| | | | 2H), 6.98-6.83 (m, 2H), 6.49 (s, 1H), 4.19 (dd, J = |
| | | | 18.9, 5.8 Hz, 4H), 3.74 (s, 3H), 2.22-2.04 (m, |
| | | | 3H), 1.69 (d, J = 11.2Hz, 2H), 1.65-1.35 (m, 4H), |
| | | | 1.13 (dd, J = 12.7, 6.6 Hz, 2H). |
| 314 | 2.4 | propionic acid | 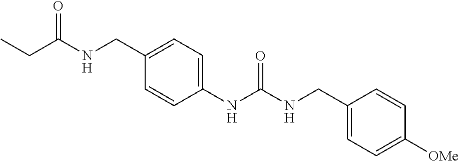 [SMILES: CCC(=O)NCc1ccc(NC(=O)NCc2ccc(OC)cc2)cc1] |
| | | | Compound 314. N-{[4-({[(4- |
| | | | methoxyphenyl)methyl]amino}carbonylamino) |
| | | | phenyl]methyl}propanamide. LCMS-ESI (POS.) |
| | | | m/z: 342.10 (M + H)+. ¹H NMR (400 MHz, |
| | | | DMSO-d₆) δ 8.46 (s, 1H), 8.16 (d, J = 6.2 Hz, |
| | | | 1H), 7.33 (d, J = 7.9 Hz, 2H), 7.23 (d, J = 8.0 Hz, |
| | | | 2H), 7.10 (d, J = 8.0 Hz, 2H), 6.96-6.83 (m, 2H), |
| | | | 6.49 (t, J = 6.0 Hz, 1H), 4.19 (dd, J = 20.0, 5.8 Hz, |
| | | | 4H), 3.74 (d, J = 1.6 Hz, 3H), 2.13 (q, J = 7.6 Hz, |
| | | | 2H), 1.02 (td, J = 2.2, 1.6 Hz, 3H) |
| 223 | 2.4 | picolinic acid | 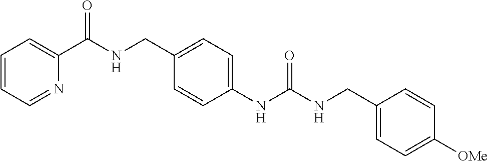 [SMILES: COc1ccc(CNC(=O)Nc2ccc(CNC(=O)c3ccccn3)cc2)cc1] |
| | | | Compound 223. {1(4- |
| | | | methoxyphenyl)methyl]amino}-N-{4-[(2- |
| | | | pyridylcarbonylamino)methyl]phenyl}carboxamide. |
| | | | LCMS-ESI (POS.) m/z: 391.00 (M + H)+. ¹H |
| | | | NMR (400 MHz, DMSO-d₆) δ 9.28-9.11 (m, 1H), |
| | | | 8.65 (d, J = 4.7 Hz, 1H), 8.46 (s, 1H), 8.03 (dt, J = |
| | | | 15.3, 7.8 Hz, 2H), 7.61 (t, J = 6.2 Hz, 1H), 7.34 (d, |
| | | | J = 8.0 Hz, 2H), 7.21 (t, J = 9.2 Hz, 4H), 6.97-6.78 |
| | | | (m, 2H), 6.47 (t, J = 5.5 Hz, 1H), 4.42 (d, J = 6.3 |
| | | | Hz, 2H), 4.21 (d, J = 5.7 Hz, 2H), 3.73 (d, J = 1.6 |
| | | | Hz, 3H). |
| 239 | 2.4 | nicotinic acid | 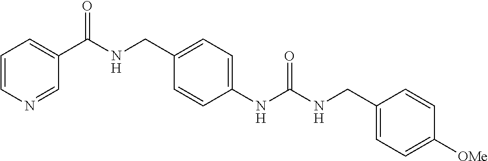 [SMILES: COc1ccc(CNC(=O)Nc2ccc(CNC(=O)c3cccnc3)cc2)cc1] |
| | | | Compound 239. {[(4- |
| | | | methoxyphenyl)methyl]amino}-N-{4-[(3- |
| | | | pyridylcarbonylamino)methyl]phenyl}carboxamide. |
| | | | LCMS-ESI (POS.) m/z: 391.10 (M + H)+. ¹H |
| | | | NMR (400 MHz, DMSO-d₆) δ 9.16 (t, J = 5.6 Hz, |
| | | | 1H), 9.04 (s, 1H), 8.71 (d, J = 4.7 Hz, 1H), 8.48 (s, |
| | | | 1H), 8.29-8.15 (m, 1H), 7.52 (dd, J =7.9, 5.1 Hz, |
| | | | 1H), 7.36 (d, J = 8.0 Hz, 2H), 7.21 (dd, J = 10.9, |
| | | | 8.2 Hz, 4H), 6.97-6.81 (m, 2H), 6.57-6.41 (m, |
| | | | 1H), 4.42 (d, J = 5.8 Hz, 2H), 4.22 (d, J = 5.7 Hz, |
| | | | 2H), 3.74 (d, J = 1.7 Hz, 3H). |
| 199 | 2.4 | 6- methylnicotinic acid | 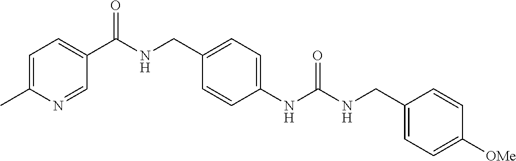 [SMILES: COc1ccc(CNC(=O)Nc2ccc(CNC(=O)c3ccc(C)nc3)cc2)cc1] |
| | | | Compound 199. {[(4- |
| | | | methoxyphenyl)methyl] amino}-N-(4-{[(6- |
| | | | methyl(3-pyridyl)) |
| | | | carbonylamino]methyl}phenyl)carboxamide. |
| | | | LCMS-ESI (POS.) m/z: 405.10 (M + H)+. |
| | | | ¹H NMR (400 MHz, DMSO-d₆) δ 9.07 (t, J = 6.0 |
| | | | Hz, 1H), 8.93 (s, 1H), 8.47 (s, 1H), 8.12 (dd, J = |
| | | | 8.1,2.5 Hz, 1H), 7.43-7.31 (m, 3H), 7.21 (dd, J = |
| | | | 15.2, 8.1 Hz, 4H), 6.90 (d, J = 8.1 Hz, 2H), 6.48 (t, |
| | | | J = 5.9 Hz, 1H), 4.40 (d, J = 5.8 Hz, 2H), 4.21 (d, J = |
| | | | 5.6 Hz, 2H), 3.73 (d, J = 1.6 Hz, 3H), 3.33 (s, 3H). |
| 323 | 2.4 | 2-(pyridin- 3-yl)acetic acid | 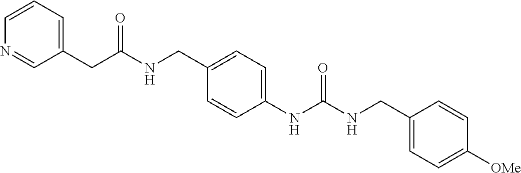 [SMILES: COc1ccc(CNC(=O)Nc2ccc(CNC(=O)Cc3cccnc3)cc2)cc1] |
| | | | Compound 323. N-{[4-({[(4- |
| | | | methoxyphenyl)methyl]amino}carbonylamino) |
| | | | phenyl]methyl}-2-(3-pyridyl)acetamide. LCMS- |
| | | | ESI (POS.) m/z: 405.05 (M + H)+. ¹H NMR (400 |
| | | | MHz, DMSO-d₆) δ 8.54 (d, J = 6.8 Hz, 1H), 8.46 |
| | | | (t, J = 6.2 Hz, 3H), 7.69 (d, J = 7.9 Hz, 1H), 7.33 |
| | | | (d, J = 8.1 Hz, 3H), 7.23 (d, J = 8.1 Hz, 2H), 7.09 |
| | | | (d, J = 8.0 Hz, 2H), 6.90 (d, J = 8.1 Hz, 2H), 6.49 |
| | | | (t, J = 6.1 Hz, 1H), 4.20 (dd, J = 11.3, 5.7 Hz, 4H), |
| | | | 3.74 (s, 3H), 3.51 (s, 2H). |
| 293 | 2.4 | 2-(6- methylpyridin- 3-yl)acetic acid | 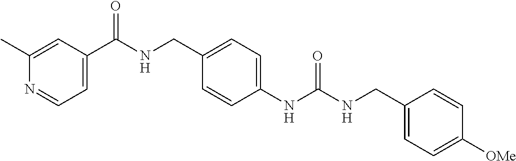 [SMILES: COc1ccc(CNC(=O)Nc2ccc(CNC(=O)c3ccnc(C)c3)cc2)cc1] |
| | | | Compound 293. N-{[4-([(4- |
| | | | methoxyphenyl)methyl]amino}carbonylamino) |
| | | | phenyl]methyl}-2-(6-methyl(3- |
| | | | pyridyl))acetamide. LCMS-ESI (POS.) m/z: |
| | | | 419.15 (M + H)+. ¹H NMR (400 MHz, DMSO-d₆) δ |
| | | | 8.48 (d, J = 14.2 Hz, 2H), 8.32 (s, 1H), 7.56 (d, J = |
| | | | 8.0 Hz, 1H), 7.33 (d, J = 8.1 Hz, 2H), 7.21 (dd, J = |
| | | | 17.1, 8.1 Hz, 3H), 7.09 (d, J = 8.0 Hz, 2H), 6.90 (d, |
| | | | J = 8.2 Hz, 2H), 6.55-6.42 (m, 1H), 4.20 (dd, J = |
| | | | 15.5, 5.8 Hz, 4H), 3.74 (d, J = 1.6 Hz, 3H), 3.45 (s, |
| | | | 2H), 2.44 (s, 3H). |
| 275 | 2.4 | (S)- tetrahydrofuran- 3-carboxylic acid | 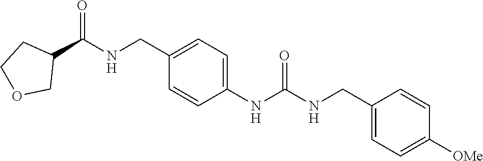 |
| | | | Compound 275. N-(4-{[((3S)oxolan-3- |
| | | | yl)carbonylamino]methyl}phenyl){[(4- |
| | | | methoxyphenyl)methyl]amino}carboxamide. |
| | | | LCMS-ESI (POS.) m/z: 384.10 (M + H)+. ¹H NMR |
| | | | (400 MHz, DMSO-d₆) δ 8.46 (s, 1H), 8.36 (s, 1H), |
| | | | 7.34 (d, J = 7.7 Hz, 2H), 7.23 (d, J = 8.0 Hz, 2H), |
| | | | 7.10 (d, J = 8.0 Hz, 2H), 6.98-6.80 (m, 2H), 6.49 |
| | | | (t, J = 6.1 Hz, 1H), 4.20 (dd, J = 11.8, 5.8 Hz, 5H), |
| | | | 3.86 (t, J = 8.1 Hz, 1H), 3.74 (s, 3H), 3.65 (dt, J = |
| | | | 16.6, 7.7 Hz, 2H), 2.97 (p, J = 8.2 Hz, 1H), 1.99 (q, |
| | | | J = 7.2 Hz, 2H). |
| 301 | 2.4 | (R)- tetrahydrofuran- 3-carboxylic acid | 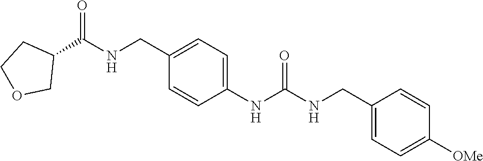 [SMILES: COc1ccc(CNC(=O)Nc2ccc(CNC(=O)[C@H]3CCOC3)cc2)cc1] |
| | | | Compound 301. N-(4-{[((3R)oxolan-3- |
| | | | yl)carbonylamino]methyl}phenyl){[(4- |
| | | | methoxyphenyl)methyl]amino}carboxamide. |
| | | | LCMS-ESI (POS.) m/z: 384.10 (M + H)+. ¹H NMR |
| | | | (400 MHz, DMSO-d₆) δ 8.46 (s, 1H), 8.36 (s, 1H), |
| | | | 7.34 (d, J = 8.1 Hz, 2H), 7.23 (d, J = 8.0 Hz, 2H), |
| | | | 7.10 (d, J = 8.0 Hz, 2H), 6.95-6.86 (m, 2H), 6.49 |
| | | | (t, J = 6.1 Hz, 1H), 4.20 (dd, J = 11.7, 5.8 Hz, 4H), |
| | | | 3.86 (t, J = 8.2 Hz, 1H), 3.74 (d, J = 1.8 Hz, 4H), |
| | | | 3.65 (dt, J = 18.2, 7.6 Hz, 2H), 3.07-2.86 (m, 1H), |
| | | | 1.99 (q, J = 7.1 Hz, 2H). |
| 194 | 2.6 | oxetane-3- carboxylic acid | 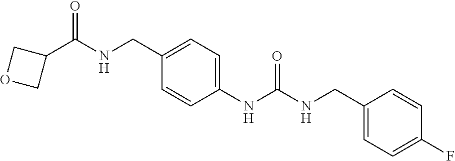 [SMILES: O=C(NCc1ccc(F)cc1)Nc1ccc(CNC(=O)C2COC2)cc1] |
| | | | Compound 194. {[(4- |
| | | | fluorophenyl)methyl]amino}-N-{4-[(oxetan-3- |
| | | | ylcarbonylamino)methyl]phenyl}carboxamide. |
| | | | LCMS-ESI (POS.) m/z: 358.05 (M + H)+. ¹H NMR |
| | | | (400 MHz, DMSO-d₆) δ 8.53 (s, 1H), 8.30 (s, 1H), |
| | | | 7.34 (d, J = 7.9 Hz, 4H), 7.27-7.04 (m, 4H), 6.59 |
| | | | (t, J = 6.0 Hz, 1H), 4.63 (d, J = 7.5 Hz, 4H), 4.24 |
| | | | (dd, J = 25.4, 5.9 Hz, 4H), 3.78 (p, J = 7.6 Hz, 1H). |
| 165 | 2.6 | 3-methyloxetane- 3-carboxylic acid | 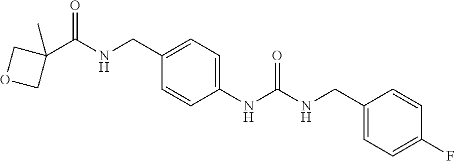 [SMILES: CC1(C(=O)NCc2ccc(NC(=O)NCc3ccc(F)cc3)cc2)COC1] |
| | | | Compound 165. {[(4- |
| | | | fluorophenyl)methyl] amino}-N-(4-{[(3- |
| | | | methyloxetan-3- |
| | | | yl)carbonylamino]methyl}phenyl)carboxamide. |
| | | | LCMS-ESI (POS.) m/z: 372.05 (M + H)+. ¹H NMR |
| | | | (400 MHz, DMSO-d₆) δ 8.52 (s, 1H), 8.36-8.24 |
| | | | (m, 1H), 7.35 (d, J = 7.7 Hz, 4H), 7.24-7.00 (m, |
| | | | 4H), 6.60 (t, J = 6.2 Hz, 1H), 4.73 (d, J = 5.9 Hz, |
| | | | 2H), 4.35-4.12 (m, 6H), 1.51 (s, 3H). |
| 176 | 2.6 | cyclopentane- carboxylic acid | 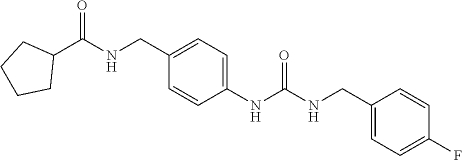 [SMILES: O=C(NCc1ccc(F)cc1)Nc1ccc(CNC(=O)C2CCCC2)cc1] |
| | | | Compound 176. N-{4- |
| | | | [(cyclopentylcarbonylamino)methyl]phenyl}{[(4- |
| | | | fluorophenyl)methyl]amino}carboxamide. |
| | | | LCMS-ESI (POS.) m/z: 370.10 (M + H)+. ¹H NMR |
| | | | (400 MHz, DMSO-d₆) δ 8.51 (s, 1H), 8.19 (d, J = |
| | | | 6.8 Hz, 1H), 7.33 (d, J = 7.5 Hz, 4H), 7.23-7.03 |
| | | | (m, 4H), 6.59 (t, J = 6.2 Hz, 1H), 4.22 (dd, J = 41.7, |
| | | | 5.9 Hz, 4H), 2.59 (t, J = 7.8 Hz, 1H), 1.85-1.69 |
| | | | (m, 2H), 1.63 (s, 4H), 1.50 (s, 2H). |
| 192 | 2.6 | (S)- tetrahydrofuran- 2-carboxylic acid | 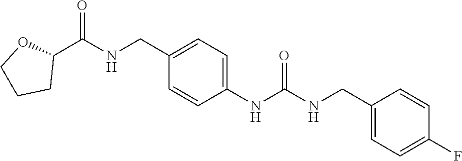 [SMILES: O=C(NCc1ccc(F)cc1)Nc1ccc(CNC(=O)[C@@H]2CCCO2)cc1] |
| | | | Compound 192. N-(4-{[((2S)oxolan-2- |
| | | | yl)carbonylamino]methyl}phenyl){[(4- |
| | | | fluorophenyl)methyl]amino}carboxamide. |
| | | | LCMS-ESI (POS.) m/z: 372.10 (M + H)+. ¹H NMR |
| | | | (400 MHz, DMSO-d₆) δ 8.51 (s, 1H), 8.30-8.17 |
| | | | (m, 1H), 7.34 (t, J = 12 Hz, 4H), 7.20-7.12 (m, |
| | | | 2H), 7.12-7.07 (m, 2H), 6.59 (t, J = 62 Hz, 1H), |
| | | | 4.33-4.12 (m, 5H), 3.90 (q, J = 6.9 Hz, 1H), 3.76 |
| | | | (q, J = 7.0 Hz, 1H), 2.20-2.05 (m, 1H), 1.94-1.72 |
| | | | (m, 3H). |
| 191 | 2.6 | (R)- tetrahydrofuran- 2-carboxylic acid | 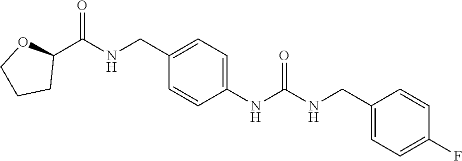 [SMILES: O=C(NCc1ccc(F)cc1)Nc1ccc(CNC(=O)[C@H]2CCCO2)cc1] |
| | | | Compound 191. N-(4-{[((2R)oxolan-2- |
| | | | yl)carbonylamino]methyl}phenyl){[(4- |
| | | | fluorophenyl)methyl]amino}carboxamide. |
| | | | LCMS-ESI (POS.) m/z: 372.10 (M + H)+. ¹H NMR |
| | | | (400 MHz, DMSO-d₆) δ 8.51 (s, 1H), 8.23 (t, J = |
| | | | 6.2 Hz, 1H), 7.33 (d, J = 7.7 Hz, 4H), 7.20-7.13 |
| | | | (m, 2H), 7.10 (d, J = 8.2 Hz, 2H), 6.59 (t, J = 6.2 |
| | | | Hz, 1H), 4.31-4.15 (m, 5H), 3.90 (q, J = 6.9 Hz, |
| | | | 1H), 3.76 (q, J = 7.1 Hz, 1H), 2.21-2.06 (m, 1H), |
| | | | 1.93-1.73 (m, 3H). |
| 187 | 2.6 | tetrahydrofuran- 3-carboxylic acid | 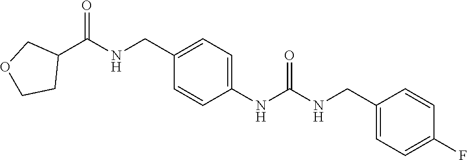 [SMILES: O=C(NCc1ccc(F)cc1)Nc1ccc(CNC(=O)C2CCOC2)cc1] |
| | | | Compound 187. {[(4- |
| | | | flnorophenyl)methyl]amino}-N-{4-[(oxolan-3- |
| | | | ylcarbonylamino)methyl]phenyl}carboxamide. |
| | | | LCMS-ESI (POS.) m/z: 372.05 (M + H)+. ¹H NMR |
| | | | (400 MHz, DMSO-d₆) δ 8.52 (s, 1H), 8.36 (t, J = |
| | | | 6.0 Hz, 1H), 7.35 (d, J = 7.8 Hz, 4H), 7.16 (t, J = |
| | | | 8.3 Hz, 2H), 7.10 (d, J = 8.2 Hz, 2H), 6.59 (t, J = |
| | | | 62 Hz, 1H), 4.23 (dd, J = 34.7, 5.8 Hz, 4H), 3.86 |
| | | | (t, J = 8.2 Hz, 1H), 3.74 (q, J = 7.3 Hz, 1H), 3.70- |
| | | | 3.58 (m, 2H), 2.97 (p, J = 7.7 Hz, 1H), 1.99 (q, J = |
| | | | 7.3 Hz, 2H). |
| 180 | 2.6 | tetrahydro- 2H-pyran-3- carboxylic acid | 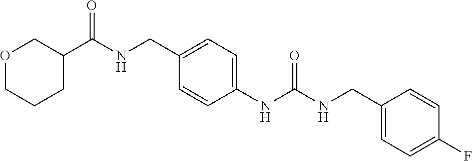 [SMILES: O=C(NCc1ccc(F)cc1)Nc1ccc(CNC(=O)C2CCCOC2)cc1] |
| | | | Compound 180. {[(4- |
| | | | fluorophenyl)methyl]amino}-N-{4-[(2H-3,4,5,6- |
| | | | tetrahydropyran-3- |
| | | | ylcarbonylamino)methyl]phenyl}carboxamide. |
| | | | LCMS-ESI (POS.) m/z: 386.10 (M + H)+. ¹H NMR |
| | | | (400 MHz, DMSO-d₆) δ 8.51 (s, 1H), 8.28 (t, J = |
| | | | 6.1 Hz, 1H), 7.33 (d, J = 7.6 Hz, 4H), 7.16 (t, J = |
| | | | 8.3 Hz, 2H), 7.08 (d, J = 8.0 Hz, 2H), 6.59 (t, J = |
| | | | 6.0 Hz, 1H), 4.27 (d, J = 5.9 Hz, 2H), 4.16 (d, J = |
| | | | 5.8 Hz, 2H), 3.91-3.71 (m, 2H), 3.40-3.21 (m, |
| | | | 2H), 2.48-2.40 (m, 1H), 1.84 (d, J = 12.9Hz, 1H), |
| | | | 1.75-1.42 (m, 3H). |
| 179 | 2.6 | tetrahydro- 2H-pyran-4- carboxylic acid | 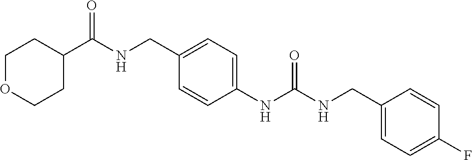 [SMILES: O=C(NCc1ccc(F)cc1)Nc1ccc(CNC(=O)C2CCOCC2)cc1] |
| | | | Compound 179. {[(4- |
| | | | fluorophenyl)methyl]amino}-N-{4-[(2H-3,4,5,6- |
| | | | tetrahydropyran-4- |
| | | | ylcarbonylamino)methyl]phenyl}carboxamide. |
| | | | LCMS-ESI (POS.) m/z: 386.10 (M + H)+. ¹H NMR |
| | | | (400 MHz, DMSO-d₆) δ 8.52 (s, 1H), 8.22 (s, 1H), |
| | | | 7.34 (t, J = 6.7 Hz, 4H), 7.16 (dd, J = 9.5, 7.6 Hz, |
| | | | 2H), 7.09 (d, J = 8.0 Hz, 2H), 6.60 (t, J = 6.0 Hz, |
| | | | 1H), 4.22 (dd, J = 39.2, 5.9 Hz, 4H), 3.86 (d, J = |
| | | | 11.3 Hz, 2H), 3.32-3.25 (m, 2H), 2.40 (t, J = 7.8 |
| | | | Hz, 1H), 1.61 (q, J = 7.0, 6.0 Hz, 4H). |
| 174 | 2.4 | cyclohexane carboxylic acid | 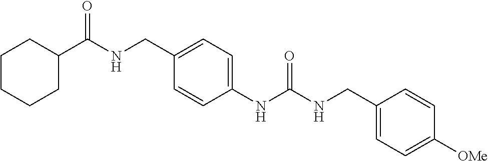 [SMILES: COc1ccc(CNC(=O)Nc2ccc(CNC(=O)C3CCCCC3)cc2)cc1] |
| | | | Compound 174. N-{4- |
| | | | [(cyclohexylcarbonylamino)methyl]phenyl}{[(4- |
| | | | fluorophenyl)methyl]amino}carboxamide. |
| | | | LCMS-ESI (POS.) m/z: 384.10 (M + H)+. ¹H NMR |
| | | | (400 MHz, DMSO-d₆) δ 8.51 (s, 1H), 8.12 (s, 1H), |
| | | | 7.34 (t, J = 7.3 Hz, 4H), 7.16 (t, J = 8.3 Hz, 2H), |
| | | | 7.08 (d, J = 8.0 Hz, 2H), 6.60 (t, J = 6.1 Hz, 1H), |
| | | | 4.21 (dd, J = 47.3, 5.9 Hz, 4H), 2.14 (t, J = 11.7 Hz, |
| | | | 1H), 1.71 (d, J = 11.0 Hz, 4H), 1.62 (d, J = 10.0 Hz, |
| | | | 1H), 1.36 (q, J = 11.9 Hz, 2H), 1.28-1.11 (m, 3H). |
| 210 | 2.4 | benzoic acid | 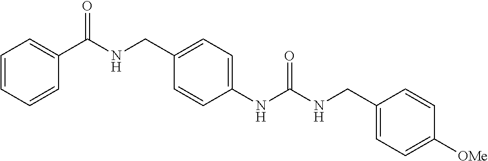 [SMILES: COc1ccc(CNC(=O)Nc2ccc(CNC(=O)c3ccccc3)cc2)cc1] |
| | | | Compound 210. {[(4- |
| | | | methoxyphenyl)methyl]amino}-N-{4- |
| | | | [(phenylcarbonylamino)methyl]phenyl}carboxamide. |
| | | | LCMS-ESI (POS.) m/z: 390.10 (M + H)+. ¹H |
| | | | NMR (400 MHz, Methanol-d₄) δ 7.86 (d, J = 7.6 |
| | | | Hz, 2H), 7.54 (d, J = 7.3 Hz, 1H), 7.48 (t, J = 7.5 |
| | | | Hz, 2H), 7.36 (d, J = 8.0 Hz, 2H), 7.32-7.23 (m, |
| | | | 4H), 6.90 (d, J = 8.0 Hz, 2H), 4.53 (s, 2H), 4.33 (s, |
| | | | 2H), 3.79 (s, 3H). |
| 173 | 2.6 | 3- isopropylcyclo- butane-1- carboxylic acid | 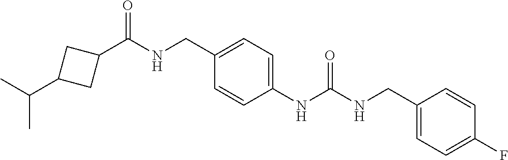 [SMILES: CC(C)C1CC(C(=O)NCc2ccc(NC(=O)NCc3ccc(F)cc3)cc2)C1] |
| | | | Compound 173. {[(4- |
| | | | fluorophenyl)methyl] amino}-N-[4-({[3- |
| | | | (methylethyl)cyclobutyl]carbonylamino}methyl) |
| | | | phenyl]carboxamide. LCMS-ESI (POS.) m/z: |
| | | | 498.10 (M + H)+. |
| | | | ¹H NMR (400 MHz, DMSO-d₆) δ 8.51 (s, 1H), 8.07 |
| | | | (s, 1H), 7.34 (t, J = 7.0 Hz, 4H), 7.16 (t, J = 8.7 Hz, |
| | | | 2H), 7.08 (t, J = 6.4 Hz, 2H), 6.59 (t, J = 6.0 Hz, |
| | | | 1H), 4.27 (d, J = 5.9 Hz, 2H), 4.16 (dd, J = 10.0, |
| | | | 6.0 Hz, 2H), 2.95-2.71 (m, 1H), 2.14 (d, J = 11.1 |
| | | | Hz, 2H), 2.08 (s, 1H), 2.01-1.89 (m, 1H), 1.87- |
| | | | 1.70 (m, 2H), 1.55-1.34 (m, 1H), 0.78 (dd, J = |
| | | | 11.2, 6.6 Hz, 5H). |
| 166 | 2.6 | cyclobutane carboxylic acid | 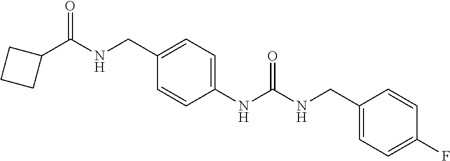 [SMILES: O=C(NCc1ccc(F)cc1)Nc1ccc(CNC(=O)C2CCC2)cc1] |
| | | | Compound 166. N-{4- |
| | | | [(cyclobutylcarbonylamino)methyllphenyl}{[(4- |
| | | | fluorophenyl)methyl]amino}carboxamide. |
| | | | LCMS-ESI (POS.) m/z: 356.05 (M + H)+. ¹H NMR |
| | | | (400 MHz, DMSO-d₆) δ 8.51 (s, 1H), 8.07 (d, J = |
| | | | 6.3 Hz, 1H), 7.34 (t, J = 6.7 Hz, 4H), 7.16 (t, J = 8.4 |
| | | | Hz, 2H), 7.09 (d, J = 8.1 Hz, 2H), 6.59 (t, J = 6.1 |
| | | | Hz, 1H), 4.21 (dd, J = 45.4, 5.9 Hz, 4H), 3.04 (p, J = |
| | | | 8.8 Hz, 1H), 2.14 (p, J = 9.4 Hz, 2H), 2.02 (d, J = |
| | | | 9.4 Hz, 2H), 1.88 (h, J = 9.1 Hz, 1H), 1.76 (d, J = |
| | | | 10.2 Hz, 1H). |
| 195 | 2.6 | propionic acid | 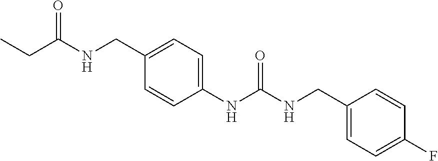 [SMILES: CCC(=O)NCc1ccc(NC(=O)NCc2ccc(F)cc2)cc1] |
| | | | Compound 195. N-{[4-([(4- |
| | | | fluorophenyl)methyl]amino}carbonylamino) |
| | | | phenyl]methyl}propanamide. LCMS-ESI (POS.) |
| | | | m/z: 330.10 (M + H)+. ¹H NMR (400 MHz, DMSO- |
| | | | d₆) δ 8.52 (s, 1H), 8.16 (d, J = 6.4 Hz, 1H), 7.34 (d, |
| | | | J = 7.7 Hz, 4H), 7.20-7.13 (m, 2H), 7.10 (d, J = |
| | | | 8.1 Hz, 2H), 6.59 (t, J = 6.2 Hz, 1H), 4.22 (dd, J = |
| | | | 43.0, 5.9 Hz, 4H), 2.13 (q, J = 7.6 Hz, 2H), 1.08- |
| | | | 0.95 (m, 3H). |
| 188 | 2.6 | cyclopropane- carboxylic acid | 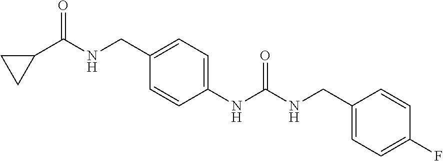 [SMILES: O=C(NCc1ccc(F)cc1)Nc1ccc(CNC(=O)C2CC2)cc1] |
| | | | Compound 188. N-{4- |
| | | | [(cyclopropylcarbonylamino)methyl]phenyl} |
| | | | {[(4-fluorophenyl)methyl]amino}carboxamide. |
| | | | LCMS-ESI (POS.) m/z: 342.05 (M + H)+. ¹H NMR |
| | | | (400 MHz, Methanol-d₄) δ 7.35 (d, J = 8.4 Hz, 4H), |
| | | | 7.21 (d, J = 8.0 Hz, 2H), 7.07 (t, J = 8.6 Hz, 2H), |
| | | | 4.35 (d, J = 21.5 Hz, 4H), 1.61 (s, 1H),0.89 (s, 2H), |
| | | | 0.82-0.74 (m, 2H). |
| 161 | 2.6 | 1- methylcyclo- propane-1- carboxylic acid | 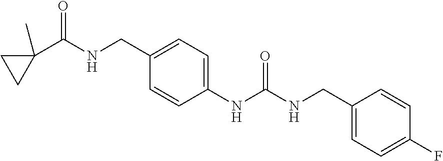 [SMILES: CC1(C(=O)NCc2ccc(NC(=O)NCc3ccc(F)cc3)cc2)CC1] |
| | | | Compound 161. {1(4- |
| | | | fluorophenyl)methyl]amino}-N-(4- |
| | | | {[(methylcyclopropyl)carbonylamino]methyl} |
| | | | phenyl)carboxamide. LCMS-ESI (POS.) m/z: |
| | | | 356.10 (M + H)+. ¹H NMR (400 MHz, DMSO-d₆) δ |
| | | | 8.51 (s, 1H), 7.99 (s, 1H), 7.44-7.26 (m, 4H), 7.16 |
| | | | (t, J = 8.6 Hz, 2H), 7.09 (d, J = 8.1 Hz, 2H), 6.60 (t, |
| | | | J = 6.0 Hz, 1H), 4.23 (dd, J = 35.9, 6.0 Hz, 4H), |
| | | | 1.28 (s, 3H), 0.96 (d, J = 2.8 Hz, 2H), 0.51 (d, J = |
| | | | 2.9 Hz, 2H). |
| 182 | 2.6 | 2- cyclopentyl acetic acid | 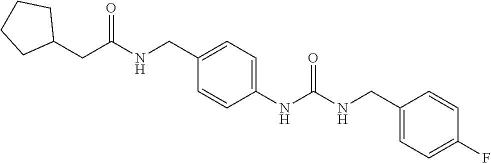 [SMILES: O=C(CC1CCCC1)NCc1ccc(NC(=O)NCc2ccc(F)cc2)cc1] |
| | | | Compound 182. 2-cyclopentyl-N-{[4-({[(4- |
| | | | fluorophenyl)methyl]amino}carbonylamino) |
| | | | phenyl]methyl}acetamide. LCMS-ESI (POS.) m/z: |
| | | | 384.10 (M + H)+. ¹H NMR (400 MHz, DMSO-d₆) δ |
| | | | 8.51 (s, 1H), 8.18 (d, J = 6.4 Hz, 1H), 7.33 (d, J = |
| | | | 7.6 Hz, 4H), 7.16 (t, 2H), 7.10 (d, J = 8.1 Hz, 2H), |
| | | | 6.63-6.56 (m, 1H), 4.22 (dd, J = 42.0, 5.8 Hz, 4H), |
| | | | 2.23-2.05 (m, 3H), 1.77-1.64 (m, 2H), 1.63- |
| | | | 1.43 (m, 4H), 1.13 (dt, J = 12.8, 6.9 Hz, 2H). |
| 169 | 2.6 | picolinic acid | 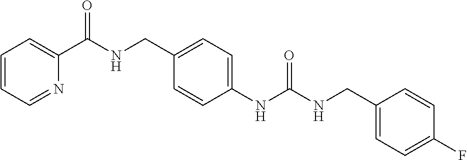 [SMILES: O=C(NCc1ccc(F)cc1)Nc1ccc(CNC(=O)c2ccccn2)cc1] |
| | | | Compound 169. {[(4- |
| | | | fluorophenyl)methyl]amino}-N-{4-[(2- |
| | | | pyridylcarbonylamino)methyl]phenyl}carboxamide. |
| | | | LCMS-ESI (POS.) m/z: 379.00 (M + H)+. ¹H |
| | | | NMR (400 MHz, DMSO-d₆) δ 9.21 (t, J = 6.4 Hz, |
| | | | 1H), 8.65 (d, J = 4.7 Hz, 1H), 8.52 (s, 1H), 8.03 (dt, |
| | | | J = 15.4, 7.8 Hz, 2H), 7.61 (t, J = 6.2 Hz, 1H), 7.34 |
| | | | (d, J = 7.8 Hz, 4H), 7.26-7.09 (m, 4H), 6.58 (t, J = |
| | | | 6.1 Hz, 1H), 4.34 (dd, J = 59.2, 6.1 Hz, 4H). |
| 184 | 2.6 | (S)- tetrahydrofuran- 3-carboxylic acid | 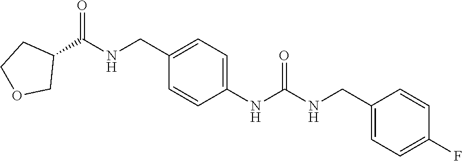 [SMILES: O=C(NCc1ccc(F)cc1)Nc1ccc(CNC(=O)[C@H]2CCOC2)cc1] |
| | | | Compound 184. N-(4-{[((3S)oxolan-3- |
| | | | yl)carbonylamino]methyl}phenyl){[(4- |
| | | | fluorophenyl)methyl]amino}carboxamide. |
| | | | LCMS-ESI (POS.) m/z: 372.10 (M + H)+. ¹H NMR |
| | | | (400 MHz, DMSO-d₆) δ 8.52 (s, 1H), 8.36 (t, J = |
| | | | 6.0 Hz, 1H), 7.35 (d, J = 7.8 Hz, 4H), 7.26-7.00 |
| | | | (m, 4H), 6.70-6.53 (m, 1H), 4.23 (dd, J = 34.6, 5.8 |
| | | | Hz, 4H), 3.86 (t, J = 8.2 Hz, 1H), 3.74 (q, J = 7.2 |
| | | | Hz, 1H), 3.65 (dt, J = 18.3, 7.6 Hz, 2H), 2.97 (p, J = |
| | | | 7.9 Hz, 1H), 1.99 (q, J = 7.1 Hz, 2H). |
| 196 | 2.6 | (R)- tetrahydrofuran- 3-carboxylic acid | 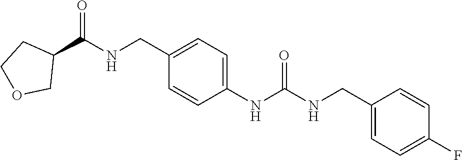 [SMILES: O=C(NCc1ccc(F)cc1)Nc1ccc(CNC(=O)[C@@H]2CCOC2)cc1] |
| | | | Compound 196. N-(4-{[((3R)oxolan-3- |
| | | | yl)carbonylamino]methyl}phenyl){[(4- |
| | | | fluorophenyl)methyl]amino}carboxamide. |
| | | | LCMS-ESI (POS.) m/z: 372.05 (M + H)+. ¹H NMR |
| | | | (400 MHz, DMSO-d₆) δ 8.52 (s, 1H), 8.36 (t, J = |
| | | | 5.9 Hz, 1H), 7.35 (d, J = 7.9 Hz, 4H), 7.16 (t, J = |
| | | | 8.6 Hz, 2H), 7.10 (d, J = 8.2 Hz, 2H), 6.59 (t, J = |
| | | | 6.1 Hz, 1H), 4.27 (d, J = 5.9 Hz, 2H), 4.19 (d, J = |
| | | | 5.8 Hz, 2H), 3.86 (t, J = 8.1 Hz, 1H), 3.80-3.57 |
| | | | (m, 3H), 2.97 (p, J = 7.9 Hz, 1H), 2.00 (t, J = 7.3 |
| | | | Hz, 2H). |
| 152 | 2.6 | 6- methylnicotinic acid | 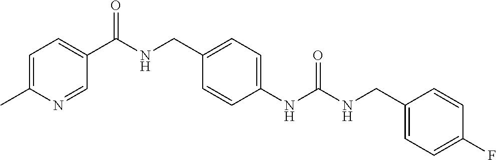 [SMILES: Cc1ccc(C(=O)NCc2ccc(NC(=O)NCc3ccc(F)cc3)cc2)cn1] |
| | | | Compound 152. {[(4- |
| | | | fluorophenyl)methyl]amino}-N-(4-{[(6- |
| | | | methyl(3-pyridyl)) |
| | | | carbonylamino]methyl}phenyl)carboxamide. |
| | | | LCMS-ESI (POS.) m/z: 393.00 (M + H)+. |
| | | | ¹H NMR (400 MHz, DMSO-d₆) δ 9.15-9.01 (m, |
| | | | 1H), 8.93 (s, 1H), 8.53 (s, 1H), 8.12 (dd, J = 8.3, |
| | | | 2.5 Hz, 1H), 7.35 (t, J = 6.3 Hz, 5H), 7.26-7.09 |
| | | | (m, 4H), 6.59 (t, J = 6.2 Hz, 1H), 4.41 (d, J = 5.9 |
| | | | Hz, 2H), 4.27 (d, J = 5.9 Hz, 2H), 3.33 (s, 3H). |
| 156 | 2.6 | benzoic acid | 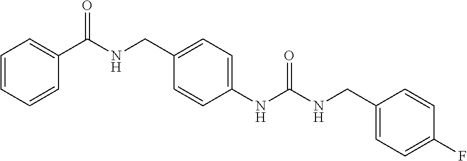 [SMILES: O=C(NCc1ccc(F)cc1)Nc1ccc(CNC(=O)c2ccccc2)cc1] |
| | | | Compound 156. {[(4- |
| | | | fluorophenyl)methyl]amino}-N-{4- |
| | | | [(phenylcarbonylamino)methyl]phenyl}carboxamide. |
| | | | LCMS-ESI (POS.) m/z: 378.00 (M + H)+. ¹H |
| | | | NMR (400 MHz, DMSO-d₆) δ 8.96 (t, J = 5.9 Hz, |
| | | | 1H), 8.53 (s, 1H), 7.89 (d, J = 7.6 Hz, 2H), 7.58- |
| | | | 7.44 (m, 3H), 7.39-7.30 (m, 4H), 7.26-7.06 (m, |
| | | | 4H), 6.59 (t, J = 6.1 Hz, 1H), 4.40 (d, J = 5.9 Hz, |
| | | | 2H), 4.27 (d, J =5.9 Hz, 2H). |
| 211 | 2.4 | isonicotinic acid | 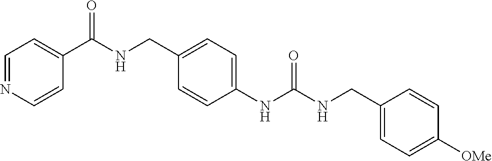 [SMILES: COc1ccc(CNC(=O)Nc2ccc(CNC(=O)c3ccncc3)cc2)cc1] |
| | | | Compound 211. {[(4- |
| | | | methoxyphenyl)methyl]amino}-N-{4-[(4- |
| | | | pyridylcarbonylamino)methyl]phenyl}carboxamide. |
| | | | LCMS-ESI (POS.) m/z: 391.10 (M + H)+. ¹H |
| | | | NMR (400 MHz, DMSO-d₆) δ 9.25 (s, 1H), 8.73 (d, |
| | | | J = 5.0 Hz, 2H), 8.48 (s, 1H), 7.79 (d, J = 4.9 Hz, |
| | | | 2H), 7.36 (d, J = 8.1 Hz, 2H), 7.30-7.10 (m, 4H), |
| | | | 6.90 (d, J = 8.1 Hz, 2H), 6.48 (s, 1H), 4.41 (d, J = |
| | | | 6.0 Hz, 2H), 4.21 (d, J = 5.9 Hz, 2H), 3.74 (s, 3H). |
| 26 | 2.5 | isonicotinic acid | 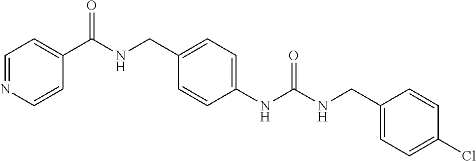 [SMILES: O=C(NCc1ccc(Cl)cc1)Nc1ccc(CNC(=O)c2ccncc2)cc1] |
| | | | Compound 26. {[(4- |
| | | | chlorophenyl)methyl] amino}-N-{4-[(4- |
| | | | pyridylcarbonylamino)methyl]phenyl}carboxamide. |
| | | | LCMS-ESI (POS.) m/z: 395.00 (M + H)+. ¹H |
| | | | NMR (400 MHz, DMSO-d₆) δ 9.27 (t, J = 6.0 Hz, |
| | | | 1H), 8.75 (dd, J = 4.5, 2.1 Hz, 2H), 8.57 (s, 1H), |
| | | | 7.87-7.72 (m, 2H), 7.52-7.27 (m, 6H), 7.20 (d, J = |
| | | | 8.1 Hz, 2H), 6.62 (t, J = 6.1 Hz, 1H), 4.42 (d, J = |
| | | | 5.9 Hz, 2H), 4.28 (d, J = 5.8 Hz, 2H). |
| 115 | 2.5 | cyclopropane- carboxylic acid | 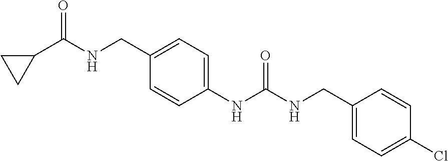 [SMILES: O=C(NCc1ccc(Cl)cc1)Nc1ccc(CNC(=O)C2CC2)cc1] |
| | | | Compound 115. {[(4- |
| | | | chlorophenyl)methyl]amino}-N-{4- |
| | | | [(cyclopropylcarbonylamino)methyl]phenyl} |
| | | | carboxamide. LCMS-ESI (POS.) m/z: 358.10 |
| | | | (M + H)+. ¹H NMR (400 MHz, DMSO-d₆) δ 8.56 (s, |
| | | | 1H), 8.45 (t, J = 5.9 Hz, 1H), 7.40 (d, J = 8.1 Hz, |
| | | | 2H), 7.37-7.28 (m, 4H), 7.11 (d, J = 8.1 Hz, 2H), |
| | | | 6.67-6.57 (m, 1H), 4.28 (d, J = 6.0 Hz, 2H), 4.19 |
| | | | (d, J = 5.8 Hz, 2H), 1.59 (t, J = 6.4 Hz, 1H), 0.74- |
| | | | 0.60 (m, 4H). |
| 41 | 2.5 | 1- methylcyclo- propane-1- carboxylic acid | 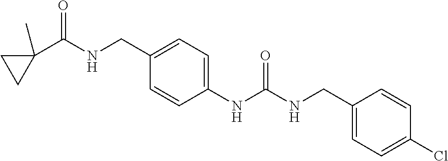 [SMILES: CC1(C(=O)NCc2ccc(NC(=O)NCc3ccc(Cl)cc3)cc2)CC1] |
| | | | Compound 41. {[(4- |
| | | | chlorophenyl)methyl]amino}-N-(4- |
| | | | {[(methyl cyclopropyl)carbonylamino]methyl} |
| | | | phenyl)carboxamide. LCMS-ESI (POS.) m/z: |
| | | | 372.10 (M + H)+. ¹H NMR (400 MHz, DMSO-d₆) δ |
| | | | 8.53 (s, 1H), 8.00 (d, J = 6.4 Hz, 1H), 7.40 (d, J = |
| | | | 8.0 Hz, 2H), 7.33 (d, J = 7.9 Hz, 4H), 7.09 (d, J = |
| | | | 8.2 Hz, 2H), 6.73-6.50 (m, 1H), 4.23 (dd, J = 39.4, |
| | | | 5.9 Hz, 4H), 1.28 (s, 3H), 0.96 (d, J = 3.0 Hz, 2H), |
| | | | 0.51 (d, J = 3.0 Hz, 2H). |
| 116 | 2.5 | 3- isopropylcyclo- butane-1- carboxylic acid | 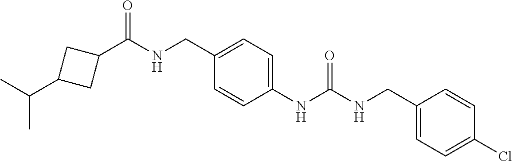 [SMILES: CC(C)C1CC(C(=O)NCc2ccc(NC(=O)NCc3ccc(Cl)cc3)cc2)C1] |
| | | | Compound 116. {[(4- |
| | | | chlorophenyl)methyl]amino}-N-[4-({[3- |
| | | | (methylethyl)cyclobutyl]carbonylamino}methyl) |
| | | | phenyl]carboxamide. LCMS-ESI (POS.) m/z: |
| | | | 414.10 (M + H)+. ¹H NMR (400 MHz, DMSO-d₆) δ |
| | | | 8.54 (s, 1H), 8.08 (d, J = 6.3 Hz, 1H), 7.40 (dd, J = |
| | | | 8.4, 1.9 Hz, 2H), 7.36-7.29 (m, 4H), 7.17-6.91 |
| | | | (m, 2H), 6.62 (t, J = 6.1 Hz, 1H), 4.28 (d, J = 6.0 |
| | | | Hz, 2H), 4.16 (dd, J = 9.9, 6.0 Hz, 2H), 2.80 (d, J = |
| | | | 9.2 Hz, 1H), 2.14 (d, J = 12.0 Hz, 1H), 2.08 (s, 1H), |
| | | | 1.96 (q, J = 7.9 Hz, 1H), 1.79 (dt, J = 29.8, 9.0 Hz, |
| | | | 2H), 1.56- 1.34 (m, 1H), 1.26 (s, 1H), 0.78 (ddd, J = |
| | | | 11.4, 6.7, 1.7 Hz, 5H). |
| 5 | 2.5 | 6- methylnicotinic acid | 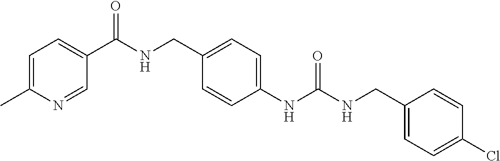 [SMILES: Cc1ccc(C(=O)NCc2ccc(NC(=O)NCc3ccc(Cl)cc3)cc2)cn1] |
| | | | Compound 5. {[(4- |
| | | | chlorophenyl)methyl]amino}-N-(4-{(6- |
| | | | methyl(3-pyridyl)) |
| | | | carbonylamino]methyl}phenyl)carboxamide. |
| | | | LCMS-ESI (POS.) m/z: 409.00 (M + H)+. |
| | | | ¹H NMR (400 MHz, DMSO-d₆) δ 9.07 (t, J = 5.9 |
| | | | Hz, 1H), 8.93 (s, 1H), 8.57 (s, 1H), 8.12 (d, J = 8.0 |
| | | | Hz, 1H), 7.36 (dq, J = 15.0, 8.0 Hz, 7H), 7.19 (d, J = |
| | | | 8.1 Hz, 2H), 6.62 (t, J =6.2 Hz, 1H), 4.41 (d, J = |
| | | | 5.8 Hz, 2H), 4.28 (d, J = 5.9 Hz, 2H), 2.09 (d, J = |
| | | | 1.6 Hz, 3H). |
| 212 | 2.4 | 2- methylnicotinic acid | 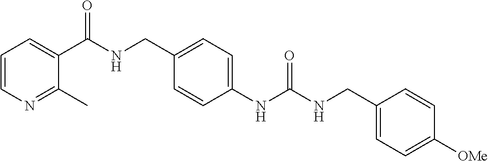 [SMILES: COc1ccc(CNC(=O)Nc2ccc(CNC(=O)c3cccnc3C)cc2)cc1] |
| | | | Compound 212. {[(4- |
| | | | methoxyphenyl)methyl] amino}-N-(4-{[(2- |
| | | | methyl(3-pyridyl)) |
| | | | carbonylamino]methyl}phenyl)carboxamide. |
| | | | LCMS-ESI (POS.) m/z: 405.10 (M + H)+. |
| | | | ¹H NMR (400 MHz, DMSO-d₆) δ 8.89 (d, J = 6.4 |
| | | | Hz, 1H), 8.59-8.38 (m, 2H), 7.75 (d, J = 7.7 Hz, |
| | | | 1H), 7.37 (d, J = 8.0 Hz, 2H), 7.30 (t, J = 7.0 Hz, |
| | | | 1H), 7.22 (t, J = 9.0 Hz, 4H), 6.99-6.83 (m, 2H), |
| | | | 6.49 (d, J = 6.4 Hz, 1H), 4.37 (d, J = 5.9 Hz, 2H), |
| | | | 4.22 (d, J = 5.7 Hz, 2H), 3.74 (s, 3H), 2.51 (s, 3H). |
| 218 | 2.4 | 4- methylnicotinic acid | 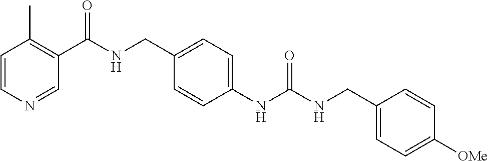 [SMILES: COc1ccc(CNC(=O)Nc2ccc(CNC(=O)c3cnccc3C)cc2)cc1] |
| | | | Compound 218.{[(4- |
| | | | methoxyphenyl)methyl]amino}-N-(4-{[(4- |
| | | | methyl(3-pyridyl)) |
| | | | carbonylamino]methyl}phenyl)carboxamide. |
| | | | LCMS-ESI (POS.) m/z: 405.10 (M + H)+. |
| | | | ¹H NMR (400 MHz, DMSO-d₆) δ 8.94 (s, 1H), 8.60- |
| | | | 8.37 (m, 3H), 7.41-7.34 (m, 2H), 7.30 (d, J = 5.0 |
| | | | Hz, 1H), 7.26-7.17 (m, 4H), 6.97-6.81 (m, 2H), |
| | | | 6.50 (t, J = 5.9 Hz, 1H), 4.38 (d, J = 5.9 Hz, 2H), |
| | | | 4.22 (d, J = 5.7 Hz, 2H), 3.74 (s, 3H), 2.36 (s, 3H). |
| 125 | 2.5 | propionic acid | 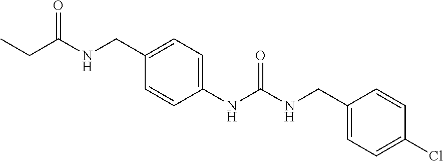 [SMILES: CCC(=O)NCc1ccc(NC(=O)NCc2ccc(Cl)cc2)cc1] |
| | | | Compound 125. N-{[4-({[(4- |
| | | | chlorophenyl)methyl]amino}carbonylamino) |
| | | | phenyl]methyl}propanamide. LCMS-ESI (POS.) |
| | | | m/z: 346.00 (M + H)+. ¹H NMR (400 MHz, DMSO- |
| | | | d₆) 8 8.55 (s, 1H), 8.17(s, 1H), 7.40 (d, J = 8.0 Hz, |
| | | | 2H), 7.36-7.30 (m, 4H), 7.10 (d, J = 8.1 Hz, 2H), |
| | | | 6.62 (t, J = 6.1 Hz, 1H), 4.28 (d, J = 5.9 Hz, 2H), |
| | | | 4.17 (d, J = 5.9 Hz, 2H), 2.12 (q, J = 7.6 Hz, 2H), |
| | | | 1.13-0.91 (m, 3H). |
| 74 | 2.5 | cyclobutane carboxylic acid | 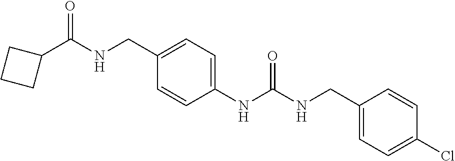 [SMILES: O=C(NCc1ccc(Cl)cc1)Nc1ccc(CNC(=O)C2CCC2)cc1] |
| | | | Compound 74. {[(4- |
| | | | chlorophenyl)methyl]amino}-N-{4- |
| | | | [(cyclobutylcarbonylamino)methyl]phenyl} |
| | | | carboxamide. LCMS-ESI (POS.) m/z: 372.05 |
| | | | (M + H)+. ¹H NMR (400 MHz, DMSO-d₆) δ 8.54 |
| | | | (s, 1H), 8.07 (d, J = 6.6 Hz, 1H), 7.40 (dd, J = 8.4, |
| | | | 1.9 Hz, 2H), 7.33 (dt, J = 7.4, 3.1 Hz, 4H), 7.09 (d, |
| | | | J = 8.1 Hz, 2H), 6.67-6.54 (m, 1H), 4.28 (d, J = |
| | | | 5.9 Hz, 2H), 4.16 (d, J = 5.9 Hz, 2H), 3.04 (p, J = |
| | | | 8.6 Hz, 1H), 2.14 (p, J = 9.5 Hz, 2H), 2.07-1.96 |
| | | | (m, 2H), 1.89 (dt, J = 18.1, 8.9 Hz, 1H), 1.77 (t, J = |
| | | | 10.1 Hz, 1H). |
| 120 | 2.5 | oxetane-3- carboxylic acid | 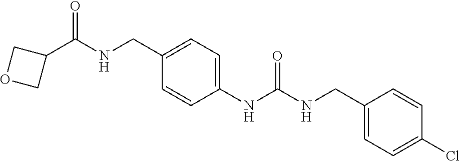 [SMILES: O=C(NCc1ccc(Cl)cc1)Nc1ccc(CNC(=O)C2COC2)cc1] |
| | | | Compound 120. {[(4- |
| | | | chlorophenyl)methyl]amino}-N-{4-[(oxetan-3- |
| | | | ylcarbonylamino)methyl]phenyl}carboxamide. |
| | | | LCMS-ESI (POS.) m/z: 374.00 (M + H)+. ¹H NMR |
| | | | (400 MHz, DMSO-d₆) δ 8.55 (d, J = 10.9 Hz, 1H), |
| | | | 8.30 (s, 1H), 7.40 (d, J = 8.3 Hz, 2H), 7.32 (d, J = |
| | | | 7.7 Hz, 4H), 7.11 (d, J = 8.1 Hz, 2H), 6.62 (s, 1H), |
| | | | 4.63 (d, J = 7.6 Hz, 3H), 4.24 (dd, J = 28.6, 5.8 Hz, |
| | | | 5H), 3.78 (t, J =7.5 Hz, 1H). |
| 85 | 2.5 | (S)- tetrahydrofuran- 3-carboxylic acid | 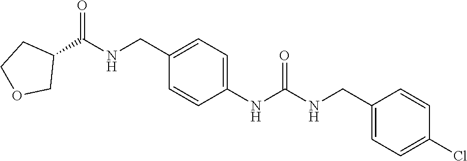 [SMILES: O=C(NCc1ccc(Cl)cc1)Nc1ccc(CNC(=O)[C@H]2CCOC2)cc1] |
| | | | Compound 85. N-(4-{[((3S)oxolan-3- |
| | | | yl)carbonylamino]methyl}phenyl){[(4- |
| | | | chlorophenyl)methyl]amino}carboxamide. |
| | | | LCMS-ESI (POS.) m/z: 388.05 (M + H)+. ¹H NMR |
| | | | (400 MHz, DMSO-d₆) δ 8.56 (s, 1H), 8.37 (d, J = |
| | | | 6.2 Hz, 1H), 7.40 (d, J = 7.8 Hz, 2H), 7.37-7.30 |
| | | | (m, 4H), 7.10 (d, J = 8.1 Hz, 2H), 6.63 (t, J = 5.8 |
| | | | Hz, 1H), 4.28 (d, J = 5.9 Hz, 2H), 4.19 (d, J = 5.8 |
| | | | Hz, 2H), 3.86 (t, J = 8.2 Hz, 1H), 3.78-3.59 (m, |
| | | | 3H), 2.96 (p, J = 7.7 Hz, 1H), 1.99 (q, J = 7.2 Hz, |
| | | | 2H). |
| 96 | 2.5 | (R)- tetrahydrofuran- 3-carboxylic acid | 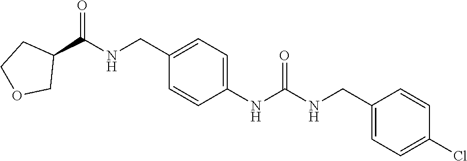 [SMILES: O=C(NCc1ccc(Cl)cc1)Nc1ccc(CNC(=O)[C@@H]2CCOC2)cc1] |
| | | | Compound 96. N-(4-{[((3R)oxolan-3- |
| | | | yl)carbonylamino]methyl}phenyl){[(4- |
| | | | chlorophenyl)methyl]amino}carboxamide. |
| | | | LCMS-ESI (POS.) m/z: 388.10 (M + H)+. ¹H NMR |
| | | | (400 MHz, DMSO-d₆) δ 8.56 (s, 1H), 8.36 (s, 1H), |
| | | | 7.40 (d, J = 8.0 Hz, 2H), 7.37-7.30 (m, 4H), 7.10 |
| | | | (d, J = 8.1 Hz, 2H), 6.63 (t, J = 6.2 Hz, 1H), 4.28 |
| | | | (d, J = 5.9 Hz, 2H), 4.19 (d, J = 5.8 Hz, 2H), 3.86 |
| | | | (t, J = 8.1 Hz, 1H), 3.78-3.58 (m, 4H), 1.99 (q, J = |
| | | | 7.4 Hz, 2H). |
| 110 | 2.5 | 2- cyclopentyl acetic acid | 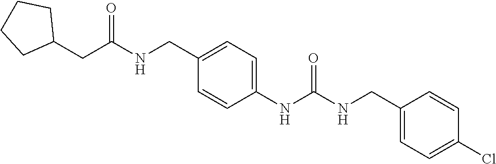 [SMILES: O=C(CC1CCCC1)NCc1ccc(NC(=O)NCc2ccc(Cl)cc2)cc1] |
| | | | Compound 110. N-{[4-({1(4- |
| | | | chlorophenyl)methyl]amino}carbonylamino) |
| | | | phenyl]methyl}-2-cyclopentylacetamide. LCMS- |
| | | | ESI (POS.) m/z: 400.10 (M + H)+. ¹H NMR (400 |
| | | | MHz, DMSO-d₆) δ 8.54 (s, 1H), 8.20 (d, J = 6.5 Hz, |
| | | | 1H), 7.43-7.37 (m, 2H), 7.36-7.29 (m, 3H), 7.10 |
| | | | (d, J = 8.1 Hz, 2H), 6.72-6.54 (m, 1H), 4.22 (dd, |
| | | | J = 45.4, 5.9 Hz, 5H), 2.22-2.07 (m, 3H), 1.76- |
| | | | 1.63 (m, 2H), 1.62-1.43 (m, 4H), 1.19-1.05 (m, |
| | | | 2H). |
| 62 | 2.5 | picolinic acid | 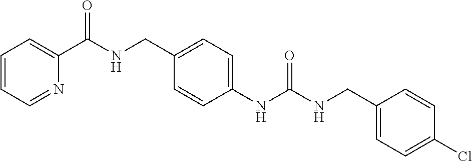 [SMILES: O=C(NCc1ccc(Cl)cc1)Nc1ccc(CNC(=O)c2ccccn2)cc1] |
| | | | Compound 62. {[(4- |
| | | | chlorophenyl)methyl]amino}-N-{4- [(2- |
| | | | pyridylcarbonylamino)methyl]phenyl}carboxamide. |
| | | | LCMS-ESI (POS.) m/z: 395.00 (M + H)+. ¹H |
| | | | NMR (400 MHz, DMSO-d₆) δ 9.21 (t, J = 6.6 Hz, |
| | | | 1H), 8.65 (d, J = 4.7 Hz, 1H), 8.56 (s, 1H), 8.03 (dt, |
| | | | J = 15.4, 7.8 Hz, 2H), 7.61 (t, J = 6.2 Hz, 1H), 7.39 |
| | | | (d, J = 8.6, 1.9 Hz, 2H), 7.36-7.29 (m, 4H), 7.20 |
| | | | (d, J = 8.1 Hz, 2H), 6.61 (t, J = 5.8 Hz, 1H), 4.35 |
| | | | (dd, J = 55.9, 6.1 Hz, 4H). |
| 44 | 2.5 | benzoic acid | 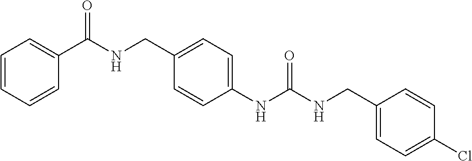 [SMILES: O=C(NCc1ccc(Cl)cc1)Nc1ccc(CNC(=O)c2ccccc2)cc1] |
| | | | Compound 44. {[(4- |
| | | | chlorophenyl)methyl]amino}-N-{4- |
| | | | [(phenylcarbonylamino)methyl]phenyl}carboxamide. |
| | | | LCMS-ESI (POS.) m/z: 394.10 (M + H)+. ¹H |
| | | | NMR (400 MHz, DMSO-d₆) δ 9.05-8.87 (m, 1H), |
| | | | 8.56 (s, 1H), 7.89 (d, J = 7.5 Hz, 2H), 7.58-7.44 |
| | | | (m, 3H), 7.42-7.30 (m, 6H), 7.19 (d, J = 8.1 Hz, |
| | | | 2H), 6.62 (t, J = 5.6 Hz, 1H), 4.34 (dd, J = 48.6, 5.9 |
| | | | Hz, 4H). |
| 155 | 2.6 | isonicotinic acid | 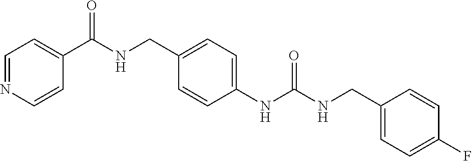 [SMILES: O=C(NCc1ccc(F)cc1)Nc1ccc(CNC(=O)c2ccncc2)cc1] |
| | | | Compound 155. {[(4- |
| | | | fluorophenyl)methyl]amino}-N-{4-[(4- |
| | | | pyridylcarbonylamino)methyl]phenyl}carboxamide. |
| | | | LCMS-ESI (POS.) m/z: 379.00 (M + H)+. ¹H |
| | | | NMR (400 MHz, DMSO-d₆) δ 9.26 (d, J = 6.2 Hz, |
| | | | 1H), 8.75 (q, J = 2.1Hz, 2H), 8.54 (s, 1H), 7.81 (dd, |
| | | | J = 4.6, 2.2 Hz, 2H), 7.43-7.27 (m, 4H), 7.27- |
| | | | 7.02 (m, 4H), 6.68-6.50 (m, 1H), 4.42 (d, J = 5.9 |
| | | | Hz, 2H), 4.27 (d, J = 5.8 Hz, 2H). |
| 145 | 2.5 | nicotinic acid | 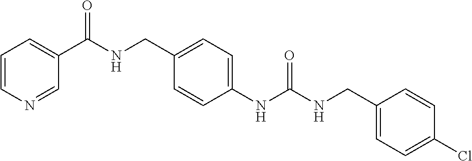 [SMILES: O=C(NCc1ccc(Cl)cc1)Nc1ccc(CNC(=O)c2cccnc2)cc1] |
| | | | Compound 145. {[(4- |
| | | | chlorophenyl)methyl]amino}-N-{4-[(3- |
| | | | pyridylcarbonylamino)methyllphenyl}carboxamide. |
| | | | LCMS-ESI (POS.) m/z: 395.00 (M + H)+. ¹H |
| | | | NMR (400 MHz, DMSO-d₆) δ 9.24-9.11 (m, 1H), |
| | | | 9.05 (s, 1H), 8.72 (d, J = 4.7 Hz, 1H), 8.57 (s, 1H), |
| | | | 8.28-8.18 (m, 1H), 7.52 (t, J = 6.5 Hz, 1H), 7.44- |
| | | | 7.28 (m, 6H), 7.20 (d, J = 8.1 Hz, 2H), 6.62 (t, J = |
| | | | 6.2 Hz, 1H), 4.42 (d, J = 5.8 Hz, 2H), 4.28 (d, J = |
| | | | 5.8 Hz, 2H). |
| 134 | 2.5 | 2-(pyridin- 3-yl)acetic acid | 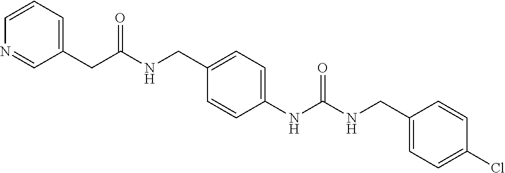 [SMILES: O=C(Cc1cccnc1)NCc1ccc(NC(=O)NCc2ccc(Cl)cc2)cc1] |
| | | | Compound 134. N-{[4-({1(4- |
| | | | chlorophenyl)methyl]amino}carbonylamino) |
| | | | phenyl]methyl}-2-(3-pyridyl)acetamide. LCMS- |
| | | | ESI (POS.) m/z: 409.05 (M + H)+. ¹H NMR (400 |
| | | | MHz, DMSO-d₆) δ 8.73 (d, J = 8.8 Hz, 2H), 8.63 |
| | | | (t, J = 6.3 Hz, 1H), 8.57 (s, 1H), 8.26 (d, J = 8.2 Hz, |
| | | | 1H), 7.84 (dd, J = 10.3, 4.2 Hz, 1H), 7.40 (d, J = |
| | | | 8.1 Hz, 3H), 7.37-7.30 (m, 4H), 7.12 (d, J = 8.0 |
| | | | Hz, 2H), 6.63 (d, J = 6.7 Hz, 1H), 4.28 (d, J = 5.1 |
| | | | Hz, 1H), 4.21 (d, J = 5.8 Hz, 2H), 3.73 (s, 2H). |
| 103 | 2.5 | tetrahydrofuran- 3-carboxylic acid | 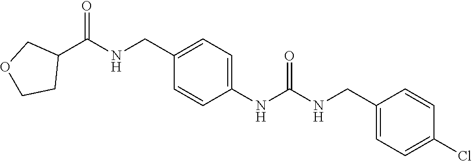 [SMILES: O=C(NCc1ccc(Cl)cc1)Nc1ccc(CNC(=O)C2CCOC2)cc1] |
| | | | Compound 103. {[(4- |
| | | | chlorophenyl)methyl] amino}-N-{4-[(oxolan-3- |
| | | | ylcarbonylamino)methyl]phenyl}carboxamide. |
| | | | LCMS-ESI (POS.) m/z: 388.00 (M + H)+. ¹H NMR |
| | | | (400 MHz, DMSO-d₆) δ 8.56 (s, 1H), 8.45-8.27 |
| | | | (m, 1H), 7.40 (dd, J = 8.5, 1.9 Hz, 2H), 7.37-7.30 |
| | | | (m, 4H), 7.10 (d, J = 7.9 Hz, 2H), 6.63 (t, J = 6.2 |
| | | | Hz, 1H), 4.28 (d, J = 5.9 Hz, 2H), 4.19 (d, J = 5.8 |
| | | | Hz, 2H), 3.86 (t, J = 8.2 Hz, 1H), 3.69 (dp, J = 32.6, |
| | | | 7.7 Hz, 3H), 2.97 (p, J = 7.5 Hz, 1H), 1.99 (q, J = |
| | | | 7.3 Hz, 2H). |
| 84 | 2.5 | cyclopentane- carboxylic acid | 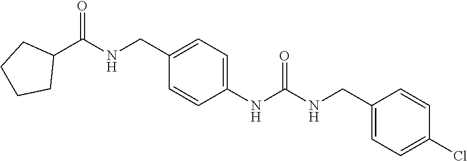 [SMILES: O=C(NCc1ccc(Cl)cc1)Nc1ccc(CNC(=O)C2CCCC2)cc1] |
| | | | Compound 84. {1(4- |
| | | | chlorophenyl)methyl]amino}-N-{4- |
| | | | [(cyclopentylcarbonylamino)methyl] phenyl} |
| | | | carboxamide. LCMS-ESI (POS.) m/z: 386.00 |
| | | | (M + H)+. ¹H NMR (400 MHz, DMSO-d₆) δ 8.54 (s, |
| | | | 1H), 8.18 (t, J = 5.9 Hz, 1H), 7.40 (d, J = 7.6 Hz, |
| | | | 2H), 7.33 (td, J = 6.0, 2.7 Hz, 4H), 7.09 (d, J = 8.1 |
| | | | Hz, 2H), 6.70-6.54 (m, 1H), 4.23 (dd, J = 44.8, |
| | | | 5.9 Hz, 4H), 2.59 (t, J = 7.7 Hz, 1H), 1.82-1.70 |
| | | | (m, 2H), 1.63 (s, 4H), 1.50 (s, 2H). |
| 82 | 2.5 | tetrahydro- 2H-pyran-4- carboxylic acid | 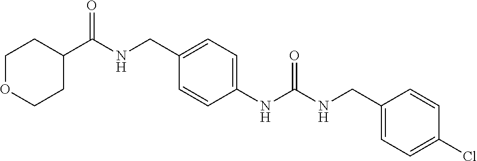 [SMILES: O=C(NCc1ccc(Cl)cc1)Nc1ccc(CNC(=O)C2CCOCC2)cc1] |
| | | | Compound 82. {[(4- |
| | | | chlorophenyl)methyl] amino}-N-{4-[(2H-3,4,5,6- |
| | | | tetrahydropyran-4- |
| | | | ylcarbonylamino)methyl]phenyl}carboxamide. |
| | | | LCMS-ESI (POS.) m/z: 402.10 (M + H)+. ¹H NMR |
| | | | (400 MHz, DMSO-d₆) δ 8.55 (s, 1H), 8.22 (t, J = |
| | | | 6.1 Hz, 1H), 7.40 (dd, J = 8.4, 1.9 Hz, 2H), 7.34 (dt, |
| | | | J = 8.7, 3.8 Hz, 4H), 7.09 (d, J = 8.1 Hz, 2H), 6.62 |
| | | | (t, J = 6.1 Hz, 1H), 4.28 (d, J = 5.9 Hz, 2H), 4.18 |
| | | | (d, J =5.9 Hz, 2H), 3.86 (d, J = 11.0 Hz, 2H), 3.32- |
| | | | 3.24 (m, 2H), 2.41 (p, J = 7.9 Hz, 1H), 1.61 (dq, |
| | | | J =6.9, 4.1 Hz, 4H). |
| 76 | 2.5 | cyclohexane carboxylic acid | 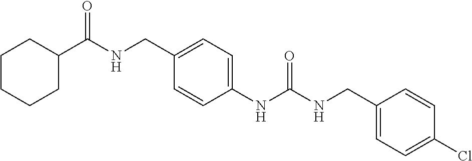 [SMILES: O=C(NCc1ccc(Cl)cc1)Nc1ccc(CNC(=O)C2CCCCC2)cc1] |
| | | | Compound 76. {[(4- |
| | | | chlorophenyl)methyl]amino}-N-{4- |
| | | | [(cyclohexylcarbonylamino)methyllphenyl} |
| | | | carboxamide. LCMS-ESI (POS.) m/z: 400.00 (M + H)+. |
| | | | ¹H NMR (400 MHz, DMSO-d₆) δ 8.54 (s, 1H), 8.12 |
| | | | (t, J = 6.1 Hz, 1H), 7.43-7.36 (m, 2H), 7.33 (dd, J = |
| | | | 8.2, 2.6 Hz, 4H), 7.08 (d, J = 8.0 Hz, 2H), 6.73- |
| | | | 6.51 (m, 1H), 4.28 (d, J = 5.9 Hz, 2H), 4.15 (d, J = |
| | | | 5.9 Hz, 2H), 2.14 (t, J = 11.3 Hz, 1H), 1.71 (d, J = |
| | | | 11.1 Hz, 4H), 1.62 (d, J = 10.5 Hz, 1H), 1.36 (q, J = |
| | | | 12.2, 11.7 Hz, 2H), 1.28- 1.09 (m, 3H). |
| 67 | 2.5 | 3- methyloxetane- 3-carboxylic acid | 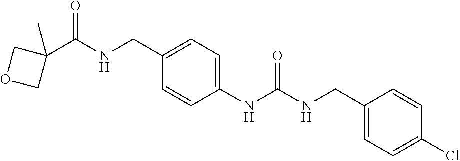 [SMILES: CC1(C(=O)NCc2ccc(NC(=O)NCc3ccc(Cl)cc3)cc2)COC1] |
| | | | Compound 67. {[(4- |
| | | | chlorophenyl)methyl]amino}-N-(4-{(3- |
| | | | methyloxetan-3- |
| | | | yl)carbonylamino]methyl}phenyl)carboxamide. |
| | | | LCMS-ESI (POS.) m/z: 388.00 (M + H)+. ¹H NMR |
| | | | (400 MHz, DMSO-d₆) δ 8.55 (s, 1H), 8.29 (s, 1H), |
| | | | 7.43-7.29 (m, 6H), 7.11 (d, J = 8.2 Hz, 2H), 6.63 |
| | | | (s, 1H), 4.81-4.67 (m, 2H), 4.34-4.17 (m, 6H), |
| | | | 1.51 (s, 3H). |
| 97 | 2.5 | (S)- tetrahydrofuran- 2-carboxylic acid | 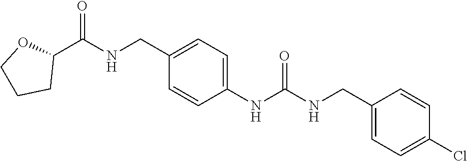 [SMILES: O=C(NCc1ccc(Cl)cc1)Nc1ccc(CNC(=O)[C@@H]2CCCO2)cc1] |
| | | | Compound 97. N-(4-{[((2S)oxolan-2- |
| | | | yl)carbonylamino]methyl}phenyl){[(4- |
| | | | chlorophenyl)methyl]amino}carboxamide. |
| | | | LCMS-ESI (POS.) m/z: 388.00 (M + H)+. ¹H NMR |
| | | | (400 MHz, DMSO-d₆) δ 8.54 (s, 1H), 8.23 (t, J = |
| | | | 6.5 Hz, 1H), 7.42-7.37 (m, 2H), 7.35-7.30 (m, |
| | | | 4H), 7.10 (d, J = 8.1 Hz, 2H), 6.62 (t, J = 5.8 Hz, |
| | | | 1H), 4.28 (d, J = 5.9 Hz, 2H), 4.26-4.22 (m, 1H), |
| | | | 4.18 (d, J = 6.3 Hz, 2H), 3.90 (q, J = 7.1 Hz, 1H), |
| | | | 3.76 (q, J = 7.1 Hz, 1H), 2.20-2.04 (m, 1H), 1.93- |
| | | | 1.71 (m, 3H). |
| 112 | 2.5 | (R)- tetrahydrofuran- 2-carboxylic acid | 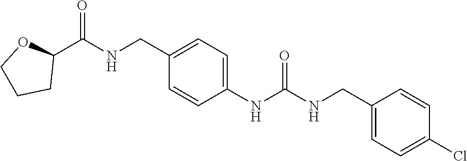 [SMILES: O=C(NCc1ccc(Cl)cc1)Nc1ccc(CNC(=O)[C@H]2CCCO2)cc1] |
| | | | Compound 112. N-(4-{[((2R)oxolan-2- |
| | | | yl)carbonylamino]methyl}phenyl){[(4- |
| | | | chlorophenyl)methyl]amino}carboxamide. |
| | | | LCMS-ESI (POS.) m/z: 388.00 (M + H)+. ¹H NMR |
| | | | (400 MHz, DMSO-d₆) 8 8.55 (s, 1H), 8.35-8.18 |
| | | | (m, 1H), 7.40 (d, J = 8.0 Hz, 2H), 7.37-7.30 (m, |
| | | | 4H), 7.08 (d, J = 8.1 Hz, 2H), 6.62 (t, J = 6.1 Hz, |
| | | | 1H), 4.28 (d, J = 5.8 Hz, 2H), 4.16 (d, J = 5.7 Hz, |
| | | | 2H), 3.93-3.67 (m, 2H), 3.31 (dt, J = 25.8, 11.0 |
| | | | Hz, 1H), 2.48-2.39 (m, 1H), 1.84 (d, J = 12.7 Hz, |
| | | | 1H), 1.59 (ddt, J = 37.8, 24.7, 12.7 Hz, 2H). |
| 87 | 2.5 | tetrahydro- 2H-pyran-3- carboxylic acid | 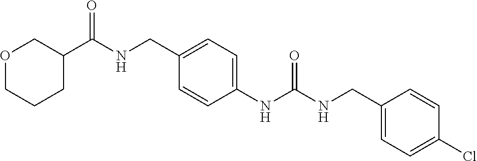 [SMILES: O=C(NCc1ccc(Cl)cc1)Nc1ccc(CNC(=O)C2CCCOC2)cc1] |
| | | | Compound 87. {[(4- |
| | | | chlorophenyl)methyl] amino}-N-{4-[(2H-3,4,5,6- |
| | | | tetrahydropyran-3- |
| | | | ylcarbonylamino)methyl]phenyl}carboxamide. |
| | | | LCMS-ESI (POS) m/z: 402.00 (M + H)+. ¹H NMR |
| | | | (400 MHz, DMSO-d₆) δ 8.54 (s, 1H), 8.23 (d, J = |
| | | | 6.8 Hz, 1H), 7.40 (dd, J = 8.5, 1.8 Hz, 2H), 7.35- |
| | | | 7.30 (m, 4H), 7.10 (d, J = 8.1 Hz, 2H), 6.62 (t, J = |
| | | | 6.0 Hz, 1H), 4.28 (d, J = 5.8 Hz, 2H), 4.23 (d, J = |
| | | | 7.0 Hz, 2H), 4.18 (d, J = 6.3 Hz, 2H), 3.90 (q, J = |
| | | | 7.0 Hz, 1H), 3.76 (q, J = 7.0 Hz, 1H), 2.13 (dq, J = |
| | | | 15.1, 7.7 Hz, 1H), 1.91-1.75 (m, 4H). |
| 219 | 2.4 | 6- isopropylnico- tinic acid | 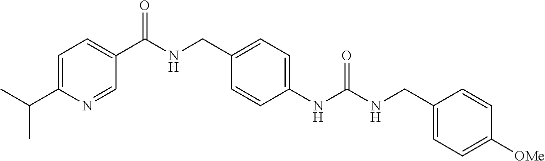 [SMILES: COc1ccc(CNC(=O)Nc2ccc(CNC(=O)c3ccc(C(C)C)nc3)cc2)cc1] |
| | | | Compound 219. {[(4- |
| | | | methoxyphenyl)methyllamino}-N-[4-({[6- |
| | | | (methylethyl)(3- |
| | | | pyridyl)]carbonylamino}methyl)phenyl]carboxamide. |
| | | | LCMS-ESI (POS.) m/z: 433.05 (M + H)+. |
| | | | ¹H NMR (400 MHz, DMSO-d₆) δ 9.06 (d, J = 6.3 |
| | | | Hz, 1H), 8.96 (s, 1H), 8.47 (s, 1H), 8.15 (d, J = 8.2 |
| | | | Hz, 1H), 7.37 (dd, J = 16.5, 8.2 Hz, 3H), 7.21 (dd, |
| | | | J = 16.3, 8.0 Hz, 4H), 6.90 (d, J = 8.0 Hz, 2H), 6.47 |
| | | | (d, J = 6.5 Hz, 1H), 4.41 (d, J = 5.9 Hz, 2H), 4.21 |
| | | | (d, J = 5.6 Hz, 2H), 3.74 (d, J = 1.8 Hz, 3H), 3.08 |
| | | | (p, J = 6.8 Hz, 1H), 1.25 (dd, J = 7.0, 1.8 Hz, 6H). |
| 160 | 2.6 | 6- isopropylnico- tinic acid | 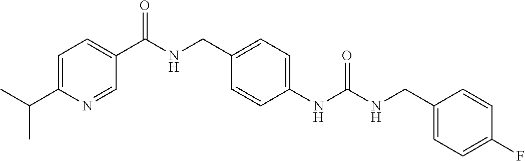 [SMILES: CC(C)c1ccc(C(=O)NCc2ccc(NC(=O)NCc3ccc(F)cc3)cc2)cn1] |
| | | | Compound 160. {[(4- |
| | | | fluorophenyl)methyl] amino}-N- [4-({[6- |
| | | | (methylethyl)(3- |
| | | | pyridyl)]carbonylamino}methyl)phenyl]carboxamide. |
| | | | LCMS-ESI (POS.) m/z: 421.20 (M + H)+. |
| | | | ¹H NMR(400 MHz, DMSO-d₆) δ 9.08 (s, 1H), 8.96 |
| | | | (s, 1H), 8.53 (s, 1H), 8.16 (d, J = 8.1 Hz, 1H), 7.37 |
| | | | (dt, J = 18.1, 8.8 Hz, 5H), 7.22-7.09 (m, 4H), 6.59 |
| | | | (d, J = 6.3 Hz, 1H), 4.41 (d, J = 5.9 Hz, 2H), 4.27 |
| | | | (d, J = 5.8 Hz, 2H), 3.09 (p, J = 7.1 Hz, 1H), 1.25 |
| | | | (d, J = 6.9 Hz, 6H). |
| 47 | 2.5 | 6- isopropylnico- tinic acid | 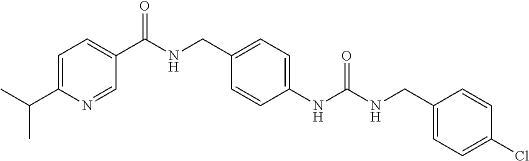 [SMILES: CC(C)c1ccc(C(=O)NCc2ccc(NC(=O)NCc3ccc(Cl)cc3)cc2)cn1] |
| | | | Compound 47. {[(4- |
| | | | chlorophenyl)methyl] amino}-N-[4-({[6- |
| | | | (methylethyl)(3- |
| | | | pyridyl)]carbonylamino}methyl)phenyl]carboxamide. |
| | | | LCMS-ESI (POS.) m/z: 437.10 (M + H)+. |
| | | | ¹H NMR (400 MHz, DMSO-d₆) δ 9.12-9.04 (m, |
| | | | 1H), 8.96 (s, 1H), 8.57 (s, 1H), 8.18 (d, J = 8.2 Hz, |
| | | | 1H), 7.47-7.28 (m, 7H), 7.19 (d, J = 8.1 Hz, 2H), |
| | | | 6.66-6.57 (m, 1H), 4.42 (d, J = 5.8 Hz, 2H), 4.28 |
| | | | (d, J = 5.7 Hz, 2H), 3.09 (p, J = 7.0 Hz, 1H), 1.26 |
| | | | (d, J = 6.9 Hz, 6H). |
| 204 | 2.7 | 6- methylnicotinic acid | 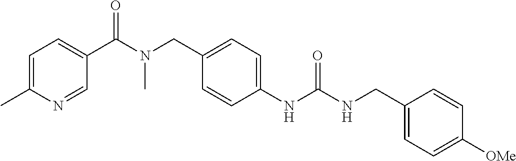 [SMILES: COc1ccc(CNC(=O)Nc2ccc(CN(C)C(=O)c3ccc(C)nc3)cc2)cc1] |
| | | | Compound 204. {1(4- |
| | | | methoxyphenyl)methyl]amino}-N-(4-{[N- |
| | | | methyl(6-methyl(3- |
| | | | pyridyl))carbonylamino]methyl}phenyl)carboxamide. |
| | | | LCMS-ESI (POS.) m/z: 419.10 (M + H)+. |
| | | | ¹H NMR (400 MHz, Methanol-d₄) δ 8.51 (s, 1H), |
| | | | 7.81 (s, 1H), 7.39 (d, J = 8.1 Hz, 4H), 7.26 (dd, J = |
| | | | 16.6, 8.2 Hz, 4H), 7.07 (d, J = 8.0 Hz, 1H), 6.95- |
| | | | 6.78 (m, 2H), 4.69 (s, 1H), 4.50 (d, J = 8.4 Hz, 1H), |
| | | | 4.31 (s, 2H), 3.77 (s, 3H), 3.03 (s, 1H), 2.92 (s, 2H), |
| | | | 2.57 (s, 3H). |
| 217 | 2.4 | 2- methylisonico- tinic acid | 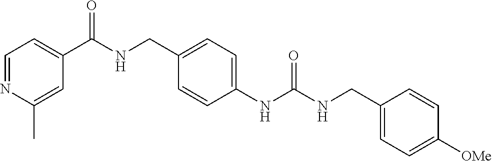 [SMILES: COc1ccc(CNC(=O)Nc2ccc(CNC(=O)c3ccnc(C)c3)cc2)cc1] |
| | | | Compound 217. {[(4- |
| | | | methoxyphenyl)methyl] amino}-N-(4-{[(2- |
| | | | methyl(4- |
| | | | pyridyl))carbonylamino]methyl}phenyl)carboxamide. |
| | | | LCMS-ESI (POS.) m/z: 405.15 (M + H)+. |
| | | | ¹H NMR (400 MHz, DMSO-d₆) δ 9.23 (d, J = 6.5 |
| | | | Hz, 1H), 8.62 (d, J = 5.2 Hz, 1H), 8.48 (s, 1H), 7.73 |
| | | | (s, 1H), 7.64 (d, J = 5.2 Hz, 1H), 7.36 (d, J = 8.1 |
| | | | Hz, 2H), 7.21 (dd, J = 15.5, 8.1 Hz, 4H), 6.93-6.86 |
| | | | (m, 2H), 6.49 (t, J = 5.8 Hz, 1H), 4.41 (d, J = 5.9 |
| | | | Hz, 2H), 4.21 (d, J = 5.6 Hz, 2H), 3.74 (d, J = 1.6 |
| | | | Hz, 3H), 2.56 (s, 3H). |
| 31 | 2.5 | 2- methylisonico- tinic acid | 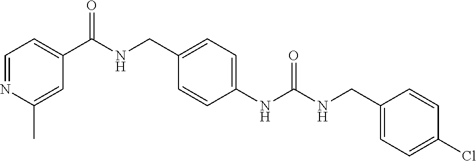 [SMILES: Cc1cc(C(=O)NCc2ccc(NC(=O)NCc3ccc(Cl)cc3)cc2)ccn1] |
| | | | Compound 31. {[(4- |
| | | | chlorophenyl)methyl] amino}-N-(4-{[(2- |
| | | | methyl(4- |
| | | | pyridyl))carbonylamino]methyl}phenyl)carboxamide. |
| | | | LCMS-ESI (POS.) m/z: 409.10 (M + H)+. |
| | | | ¹H NMR (400 MHz, DMSO-d₆) δ 9.23 (d, J = 6.4 |
| | | | Hz, 1H), 8.64-8.55 (m, 2H), 7.72 (s, 1H), 7.63 (d, |
| | | | J = 5.2 Hz, 1H), 7.36 (dt, J = 24.3, 8.0 Hz, 6H), 7.19 |
| | | | (d, J = 8.0 Hz, 2H), 6.63 (t, J = 6.1 Hz, 1H), 4.41 |
| | | | (d, J = 5.8 Hz, 2H), 4.28 (d, J = 5.8 Hz, 2H), 2.56 |
| | | | (s, 3H). |
| 154 | 2.6 | 2- methylisonico- tinic acid | 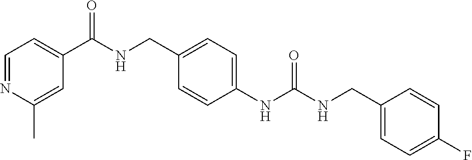 [SMILES: Cc1cc(C(=O)NCc2ccc(NC(=O)NCc3ccc(F)cc3)cc2)ccn1] |
| | | | Compound 154. {[(4- |
| | | | fluorophenyl)methyl]amino}-N-(4-{[(2- |
| | | | methyl(4-pyridyl)) |
| | | | carbonylamino]methyl}phenyl)carboxamide. |
| | | | LCMS-ESI (POS.) m/z: 393.10 (M + H)+. |
| | | | ¹H NMR (400 MHz, DMSO-d₆) δ 9.26 (d, J = 6.4 |
| | | | Hz, 1H), 8.65 (d, J = 5.3 Hz, 1H), 8.54 (s, 1H), 7.77 |
| | | | (s, 1H), 7.69 (d, J = 5.2 Hz, 1H), 7.36 (t, J = 7.2 Hz, |
| | | | 4H), 7.25-7.10 (m, 4H), 6.59 (t, J = 6.1 Hz, 1H), |
| | | | 4.41 (d, J = 5.8 Hz, 2H), 4.27 (d, J = 5.6 Hz, 2H), |
| | | | 2.58 (s, 3H). |
| 1 | 2.2 | 6- methylnicotinic acid | 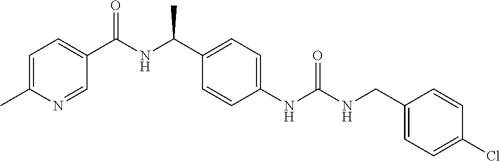 [SMILES: Cc1ccc(C(=O)N[C@@H](C)c2ccc(NC(=O)NCc3ccc(Cl)cc3)cc2)cn1] |
| | | | Compound 1. [(4-{(1S)-1-[(6-methyl(3- |
| | | | pyridyl))carbonylamino]ethyl}phenyl)amino]- |
| | | | N-[(4-chlorophenyl)methyl]carboxamide. |
| | | | LCMS-ESI (POS.) m/z: 424.10 (M + H)+. ¹H NMR |
| | | | (400 MHz, DMSO-d₆) δ 8.93 (d, J = 2.3 Hz, 1H), |
| | | | 8.86 (d, J = 8.1 Hz, 1H), 8.55 (s, 1H), 8.17 (d, J = |
| | | | 7.6 Hz, 1H), 7.43-7.37 (m, 3H), 7.37-7.30 (m, |
| | | | 4H), 7.25 (d, J = 8.4 Hz, 2H), 6.62 (t, J = 6.0 Hz, |
| | | | 1H), 5.12 (q, J = 7.4 Hz, 1H), 4.28 (d, J = 5.9 Hz, |
| | | | 2H), 2.54 (s, 3H), 1.46 (d, J = 7.0 Hz, 3H). |
| 95 | 2.3 | 6- methylnicotinic acid | 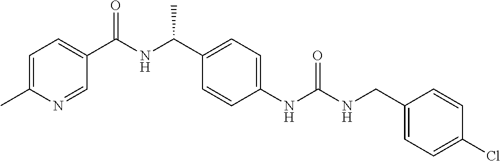 [SMILES: Cc1ccc(C(=O)N[C@H](C)c2ccc(NC(=O)NCc3ccc(Cl)cc3)cc2)cn1] |
| | | | Compound 95. [(4-{(1R)-1-[(6-methyl(3- |
| | | | pyridyl))carbonylamino]ethyl}phenyl)amino]- |
| | | | N-[(4-chlorophenyl)methyl]carboxamide. |
| | | | LCMS-ESI (POS.) m/z: 424.15 (M + H)+. ¹H NMR |
| | | | (400 MHz, DMSO-d₆) δ 8.93 (d, J = 2.3 Hz, 1H), |
| | | | 8.86 (d, J = 8.1 Hz, 1H), 8.55 (s, 1H), 8.16 (dd, J = |
| | | | 9.4, 7.3 Hz, 1H), 7.44-7.29 (m, 7H), 7.25 (d, J = |
| | | | 8.6 Hz, 2H), 6.62 (t, J = 6.0 Hz, 1H), 5.11 (p, J = |
| | | | 7.1 Hz, 1H), 4.28 (d, J = 5.8 Hz, 2H), 2.54 (s, 3H), |
| | | | 1.46 (d, J = 7.0 Hz, 3H). |
| 2 | 2.2 | cyclobutane carboxylic acid | 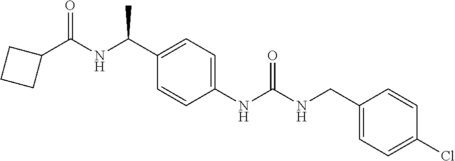 [SMILES: C[C@H](NC(=O)C1CCC1)c1ccc(NC(=O)NCc2ccc(Cl)cc2)cc1] |
| | | | Compound 2. ({4-[(1S)-1- |
| | | | (cyclobutylcarbonylamino)ethyl]phenyl}amino)- |
| | | | N-[(4-chlorophenyl)methyl]carboxamide. |
| | | | LCMS-ESI (POS.) m/z: 386.10 (M + H)+. ¹H NMR |
| | | | (400 MHz, DMSO-d₆) δ 8.53 (s, 1H), 7.96 (d, J = |
| | | | 8.2 Hz, 1H), 7.39 (dd,J = 8.4, 1.6 Hz, 2H), 7.32 (d, |
| | | | J = 8.0 Hz, 4H), 7.13 (d, J = 8.0 Hz, 2H), 6.62 (t, J = |
| | | | 6.0 Hz, 1H), 4.84 (p, J =7.1 Hz, 1H), 4.28 (d, J = |
| | | | 5.9 Hz, 2H), 3.03 (p, J = 8.4 Hz, 1H), 2.21-1.94 |
| | | | (m, 4H), 1.88 (p, J = 8.9 Hz, 1H), 1.79-1.69 (m, |
| | | | 1H), 1.29 (d, J = 7.0 Hz, 3H). |
| 46 | 2.2 | oxetane-3- carboxylic acid | 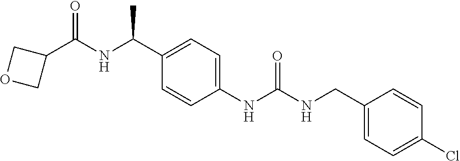 [SMILES: C[C@H](NC(=O)C1COC1)c1ccc(NC(=O)NCc2ccc(Cl)cc2)cc1] |
| | | | Compound 46. ({4-[(1S)-1-(oxetan-3- |
| | | | ylcarbonylamino)ethyl]phenyl}amino)-N- [(4- |
| | | | chlorophenyl)methyl]carboxamide. LCMS-ESI |
| | | | (POS.) m/z: 386.10 (M + H)+. ¹H NMR (400 MHz, |
| | | | DMSO-d₆) δ 8.54 (s, 1H), 8.21 (d, J = 8.1 Hz, 1H), |
| | | | 7.39 (d, J = 8.2 Hz, 2H), 7.36-7.29 (m, 4H), 7.15 |
| | | | (d, J = 8.3 Hz, 2H), 6.62 (t, J = 5.9 Hz, 1H), 4.88 (t, |
| | | | J = 7.3 Hz, 1H), 4.68-4.49 (m, 4H), 4.28 (d, J = |
| | | | 6.0 Hz, 2H), 3.76 (t, J = 7.4 Hz, 1H), 1.31 (d, J = |
| | | | 7.0 Hz, 3H). |
| 37 | 2.2 | 3- methyloxetane- 3-carboxylic acid | 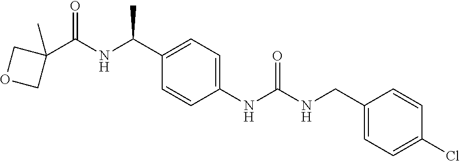 [SMILES: C[C@H](NC(=O)C1(C)COC1)c1ccc(NC(=O)NCc2ccc(Cl)cc2)cc1] |
| | | | Compound 37. [(4-{(1S)-1-[(3-methyloxetan-3- |
| | | | yl)carbonylamino]ethyl}phenyl)amino]-N-[(4- |
| | | | chlorophenyl)methyl]carboxamide. LCMS-ESI |
| | | | (POS.) m/z: 403.05 (M + H)+. ¹H NMR (400 MHz, |
| | | | DMSO-d₆) δ 8.54 (s, 1H), 8.11 (d, J = 8.0 Hz, 1H), |
| | | | 7.39 (d, J = 8.2 Hz, 2H), 7.36-7.29 (m, 4H), 7.16 |
| | | | (d, J = 8.3 Hz, 2H), 6.62 (t, J = 6.0 Hz, 1H), 4.87 |
| | | | (q, J = 7.3 Hz, 1H), 4.70 (dd, J = 6.0, 1.8 Hz, 2H), |
| | | | 4.34-4.19 (m, 4H), 1.49 (s, 3H), 1.33 (d, J = 7.0 |
| | | | Hz, 3H). |
| 60 | 2.2 | cyclohexane carboxylic acid | 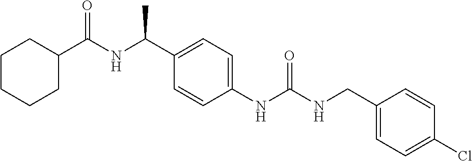 [SMILES: C[C@H](NC(=O)C1CCCCC1)c1ccc(NC(=O)NCc2ccc(Cl)cc2)cc1] |
| | | | Compound 60. ({4-[(1S)-1- |
| | | | (cyclohexylcarbonylamino)ethyl]phenyl}amino)- |
| | | | N-[(4-chlorophenyl)methyl]carboxamide. |
| | | | LCMS-ESI (POS.) m/z: 415.10 (M + H)+. ¹H NMR |
| | | | (400 MHz, DMSO-d₆) δ 8.52 (s, 1H), 8.01 (d, J = |
| | | | 8.0 Hz, 1H), 7.39 (d, J = 8.3 Hz, 2H), 7.32 (d, J = |
| | | | 8.5 Hz, 4H), 7.13 (d, J = 8.4 Hz, 2H), 6.61 (t, J = |
| | | | 6.2 Hz, 1H), 4.92-4.65 (m, 1H), 4.28 (d, J = 6.0 |
| | | | Hz, 2H), 2.14 (s, 1H), 1.77-1.57 (m, 5H), 1.40- |
| | | | 1.12 (m, 8H). |
| 54 | 2.2 | tetrahydro- 2H-pyran-3- carboxylic acid | 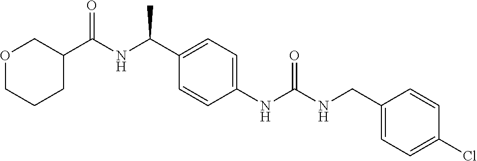 [SMILES: C[C@H](NC(=O)C1CCCOC1)c1ccc(NC(=O)NCc2ccc(Cl)cc2)cc1] |
| | | | Compound 54. ({4-[(1S)-1-(2H-3,4,5,6- |
| | | | tetrahydropyran-3- |
| | | | ylcarbonylamino)ethyl]phenyl}amino)-N-[(4- |
| | | | chlorophenyl)methyl]carboxamide. LCMS-ESI |
| | | | (POS.) m/z: 417.15 (M + H)+. ¹H NMR (400 MHz, |
| | | | DMSO-d₆) δ 8.53 (s, 1H), 8.18 (d, J = 8.1 Hz, 1H), |
| | | | 7.39 (d, J = 8.3 Hz, 2H), 7.32 (d, J = 7.9 Hz, 4H), |
| | | | 7.16-7.10 (m, 2H), 6.62 (t, J = 6.0 Hz, 1H), 4.81 |
| | | | (p, J = 7.1 Hz, 1H), 4.28 (d, J = 5.9 Hz, 2H), 3.90- |
| | | | 3.72 (m, 2H), 3.25 (ddd, J = 13.9, 7.7, 3.1 Hz, 2H), |
| | | | 2.46-2.38 (m, 1H), 1.80 (d, J = 12.1Hz, 1H), 1.65- |
| | | | 1.46 (m, 3H), 1.29 (d, J = 6.9 Hz, 3H). |
| 19 | 2.2 | tetrahydro- 2H-pyran-4- carboxylic acid | 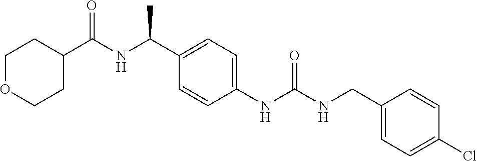 [SMILES: C[C@H](NC(=O)C1CCOCC1)c1ccc(NC(=O)NCc2ccc(Cl)cc2)cc1] |
| | | | Compound 19. ({4-[(1S)-1-(2H-3,4,5,6- |
| | | | tetrahydropyran-4- |
| | | | ylcarbonylamino)ethyl] phenyl}amino)-N- [(4- |
| | | | chlorophenyl)methyl]carboxamide. |
| | | | LCMS-ESI (POS.) m/z: 417.15 (M + H)+. ¹H NMR |
| | | | (400 MHz, DMSO-d₆) δ 8.52 (s, 1H), 8.11 (d, J = |
| | | | 8.1 Hz, 1H), 7.39 (d, J = 8.4 Hz, 2H), 7.32 (d, J = |
| | | | 8.0 Hz, 4H), 7.14 (d, J = 8.3 Hz, 2H), 6.61 (d, J = |
| | | | 5.8 Hz, 1H), 4.87-4.79 (m, 1H), 4.28 (d, J = 5.9 |
| | | | Hz, 2H), 3.85 (t, J = 7.9 Hz, 4H), 1.56 (t, J = 8.0 |
| | | | Hz, 5H), 1.30 (d, J = 7.0 Hz, 3H). |
| 14 | 2.2 | (S)- tetrahydrofuran- 2-carboxylic acid | 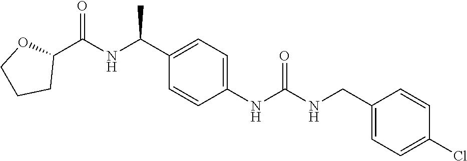 [SMILES: C[C@H](NC(=O)[C@@H]1CCCO1)c1ccc(NC(=O)NCc2ccc(Cl)cc2)cc1] |
| | | | Compound 14. [(4-{(1S)-1-[((2S)oxolan-2- |
| | | | yl)carbonylaminolethyl}phenyl)aminol-N-[(4- |
| | | | chlorophenyl)methyl]carboxamide. LCMS-ESI |
| | | | (POS.) m/z: 403.00 (M + H)+. ¹H NMR (400 MHz, |
| | | | DMSO-d₆) δ 8.54 (s, 1H), 7.94 (d, J = 8.5 Hz, 1H), |
| | | | 7.38 (s, 1H), 7.36-7.30 (m, 5H), 7.18 (d, J = 8.4 |
| | | | Hz, 2H), 6.62 (t, J = 6.1 Hz, 1H), 4.88 (p, J = 7.1 |
| | | | Hz, 1H), 4.28 (d, J = 5.9 Hz, 2H), 4.19 (dd, J = 8.2, |
| | | | 4.8 Hz, 1H), 3.91 (q, J = 6.8 Hz, 1H), 3.76 (q, J = |
| | | | 6.9 Hz, 1H), 2.16-2.02 (m, 1H), 1.88-1.73 (m, |
| | | | 3H), 1.35 (d, J = 7.0 Hz, 3H). |
| 88 | 2.2 | (R)- tetrahydrofuran- 2-carboxylic acid | 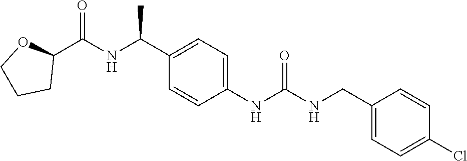 [SMILES: C[C@H](NC(=O)[C@H]1CCCO1)c1ccc(NC(=O)NCc2ccc(Cl)cc2)cc1] |
| | | | Compound 88. [(4-{1-[((2R)oxolan-2- |
| | | | yl)carbonylamino](1S)ethyl}phenyl)amino]-N- |
| | | | [(4-chlorophenyl)methyl]carboxamide. LCMS- |
| | | | ESI (POS.) m/z: 403.10 (M + H)+. ¹H NMR (400 |
| | | | MHz, DMSO-d₆) δ 8.53 (s, 1H), 7.98 (d, J = 8.5 Hz, |
| | | | 1H), 7.39 (d, J = 8.3 Hz, 2H), 7.32 (d, J = 8.0 Hz, |
| | | | 4H), 7.16 (d, J = 8.4 Hz, 2H), 6.62 (t, J = 6.0 Hz, |
| | | | 1H), 4.93-4.82 (m, 1H), 4.28 (d, J = 5.9 Hz, 2H), |
| | | | 4.22 (dd, J = 8.0, 4.4 Hz, 1H), 3.91 (t, J = 7.4 Hz, |
| | | | 1H), 3.76 (q, J = 6.7 Hz, 1H), 2.07 (dd, J = 7.9, 4.5 |
| | | | Hz, 1H), 1.85-1.71 (m, 3H), 1.36 (d, J = 7.0 Hz, |
| | | | 3H). |
| 35 | 2.2 | cyclopentane- carboxylic acid | 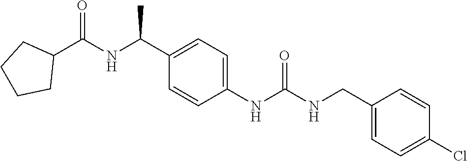 [SMILES: C[C@H](NC(=O)C1CCCC1)c1ccc(NC(=O)NCc2ccc(Cl)cc2)cc1] |
| | | | Compound 35. ({4-[(1S)-1- |
| | | | (cyclopentylcarbonylamino)ethyl]phenyl}amino)- |
| | | | N-[(4-chlorophenyl)methyl]carboxamide. |
| | | | LCMS-ESI (POS.) m/z: 401.15 (M + H)+. ¹H NMR |
| | | | (400 MHz, DMSO-d₆) δ 8.52 (s, 1H), 8.07 (d, J = |
| | | | 8.2 Hz, 1H), 7.39 (d, J = 8.2 Hz, 2H), 7.32 (d, J = |
| | | | 7.9 Hz, 4H), 7.14 (d, J = 8.3 Hz, 2H), 6.62 (t, J = |
| | | | 6.1 Hz, 1H), 4.84 (t, J = 7.3 Hz, 1H), 4.28 (d, J = |
| | | | 5.9 Hz, 2H), 2.59 (t, J = 7.7 Hz, 1H), 1.79-1.43 |
| | | | (m, 8H), 1.30 (d, J = 7.0 Hz, 3H). |
| 108 | 2.2 | benzoic acid | 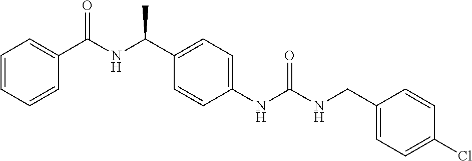 [SMILES: C[C@H](NC(=O)c1ccccc1)c1ccc(NC(=O)NCc2ccc(Cl)cc2)cc1] |
| | | | Compound 108. (4-[(1S)-1- |
| | | | (phenylcarbonylamino)ethyl]phenyl}amino)-N- |
| | | | [(4-chlorophenyl)methyl]carboxamide. LCMS- |
| | | | ESI (POS.) m/z: 409.10 (M + H)+. ¹H NMR (400 |
| | | | MHz, DMSO-d₆) δ 8.71 (d, J = 8.2 Hz, 1H), 8.54 |
| | | | (s, 1H), 8.04-7.92 (m, 3H), 7.54 (dt, J = 19.1, 7.5 |
| | | | Hz, 4H), 7.47-7.29 (m, 6H), 6.60 (d, J = 6.3 Hz, |
| | | | 1H), 5.17-5.07 (m, 1H), 4.28 (d, J = 5.9 Hz, 2H), |
| | | | 1.45 (d, J = 7.1 Hz, 3H). |
| 45 | 2.2 | picolinic acid | 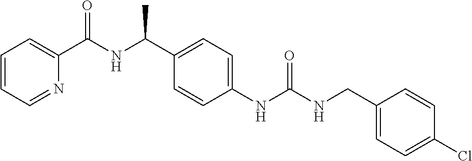 [SMILES: C[C@H](NC(=O)c1ccccn1)c1ccc(NC(=O)NCc2ccc(Cl)cc2)cc1] |
| | | | Compound 45. ({4-[(1S)-1-(2- |
| | | | pyridylcarbonylamino)ethyl]phenyl}amino)-N- |
| | | | [(4-chlorophenyl)methyl]carboxamide. LCMS- |
| | | | ESI (POS.) m/z: 409.10 (M + H)+. ¹H NMR (400 |
| | | | MHz, DMSO-d₆) δ 8.87 (s, 1H), 8.66 (d, J = 4.4 Hz, |
| | | | 2H), 8.56 (s, 2H), 8.01 (d, J = 6.9 Hz, 2H), 7.61 (s, |
| | | | 1H), 7.39 (d, J = 8.4 Hz, 3H), 7.36-7.26 (m, 4H), |
| | | | 6.61 (s, 1H), 4.28 (d, J = 6.2 Hz, 2H), 1.50 (d, J = |
| | | | 6.9 Hz, 3H). |
| 10 | 2.2 | nicotinic acid | 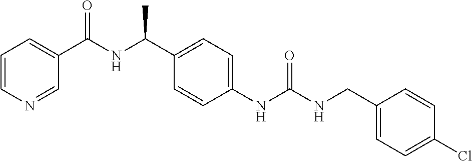 [SMILES: C[C@H](NC(=O)c1cccnc1)c1ccc(NC(=O)NCc2ccc(Cl)cc2)cc1] |
| | | | Compound 10. ({4-[(1S)-1-(3- |
| | | | pyridylcarbonylamino)ethyl]phenyl}amino)-N- |
| | | | [(4-chlorophenyl)methyl]carboxamide. LCMS- |
| | | | ESI (POS.) m/z: 409.10 (M + H)+. ¹H NMR (400 |
| | | | MHz, DMSO-d₆) δ 9.03 (d, J = 2.3 Hz, 1H), 8.93 |
| | | | (d, J = 8.0 Hz, 1H), 8.71 (dd, J = 4.9, 1.6 Hz, 1H), |
| | | | 8.55 (s, 1H), 8.22 (dt, J = 8.1, 2.1 Hz, lH),7.52(dd, |
| | | | J = 7.9, 4.8 Hz, 1H), 7.38 (dd, J = 9.1, 6.9 Hz, 3H), |
| | | | 7.35-7.30 (m, 3H), 7.26 (d, J = 8.4 Hz, 2H), 6.62 |
| | | | (t, J = 6.0 Hz, 1H), 5.11 (q, J = 7.2 Hz, 1H), 4.28 |
| | | | (d, J = 5.8 Hz, 2H), 1.47 (d, J = 7.0 Hz, 3H). |
| 9 | 2.2 | isonicotinic acid | 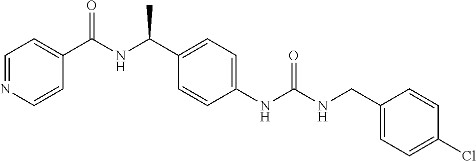 [SMILES: C[C@H](NC(=O)c1ccncc1)c1ccc(NC(=O)NCc2ccc(Cl)cc2)cc1] |
| | | | Compound 9. ({4-[(1S)-1-(4- |
| | | | pyridylcarbonylamino)ethyl]phenyl}amino)-N- |
| | | | [(4-chlorophenyl)methyl]carboxamide. LCMS- |
| | | | ESI (POS.) m/z: 409.10 (M + H)+. ¹H NMR (400 |
| | | | MHz, DMSO-d₆) δ 9.05 (d, J = 8.0 Hz, 1H), 8.79- |
| | | | 8.71 (m, 2H), 8.56 (s, 1H), 7.87-7.80 (m, 2H), |
| | | | 7.38 (t, J = 8.8, 6.7 Hz, 3H), 7.35-7.30 (m, 3H), |
| | | | 7.25 (d, J = 8.4 Hz, 2H), 6.62 (t, J = 6.0 Hz, 1H), |
| | | | 5.11 (p, J = 7.2 Hz, 1H), 4.28 (d, J = 5.7 Hz, 2H), |
| | | | 1.47 (d, J = 7.0 Hz, 3H). |
| 7 | 2.2 | 4- methylnicotinic acid | 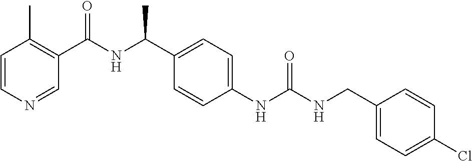 [SMILES: Cc1ccncc1C(=O)N[C@@H](C)c1ccc(NC(=O)NCc2ccc(Cl)cc2)cc1] |
| | | | Compound 7. [(4-{(1S)-1-[(4-methyl(3- |
| | | | pyridyl))carbonylamino]ethyl}phenyl)amino]- |
| | | | N-[(4-chlorophenyl)methyl] carboxamide. |
| | | | LCMS-ESI (POS.) m/z: 424.10 (M + H)+. ¹H NMR |
| | | | (400 MHz, DMSO-d₆) δ 8.86 (d, J = 8.1 Hz, 1H), |
| | | | 8.56 (s, 1H), 8.48 (d, J = 6.0 Hz, 2H), 7.44-7.28 |
| | | | (m, 7H), 7.26 (d, J = 8.4 Hz, 2H), 6.62 (d, J = 6.1 |
| | | | Hz, 1H), 5.07 (t, J = 7.8 Hz, 1H), 4.29 (d, J = 6.0 |
| | | | Hz, 2H), 2.31 (s, 3H), 1.42 (d, J = 7.0 Hz, 3H). |
| 39 | 2.2 | 2- methylnicotinic acid | 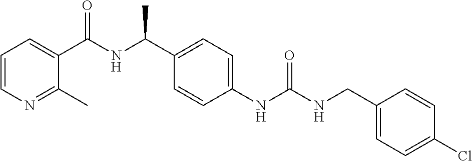 [SMILES: Cc1ncccc1C(=O)N[C@@H](C)c1ccc(NC(=O)NCc2ccc(Cl)cc2)cc1] |
| | | | Compound 39. [(4-{(1S)-1-[(2-methyl(3- |
| | | | pyridyl))carbonylaminolethyl}phenyl)amino]- |
| | | | N-[(4-chlorophenyl)methyl]carboxamide. |
| | | | LCMS-ESI (POS.) m/z: 424.10 (M + H)+. ¹H NMR |
| | | | (400 MHz, DMSO-d₆) δ 8.80 (d, J = 8.2 Hz, 1H), |
| | | | 8.56 (s, 1H), 8.49 (dd, J = 4.9, 1.7 Hz, 1H), 7.69 |
| | | | (dd,J =7.6, 1.7 Hz, 1H), 7.44-7.20 (m, 9H), 6.63 |
| | | | (t, J = 6.0 Hz, 1H), 5.13-4.98 (m, 1H), 4.28 (d, J = |
| | | | 6.0 Hz, 2H), 2.46 (s, 3H), 1.41 (d, J = 7.0 Hz, 3H). |
| 473 | 2.5 | (S)-1- methylazetidine- 2-carboxylic acid | 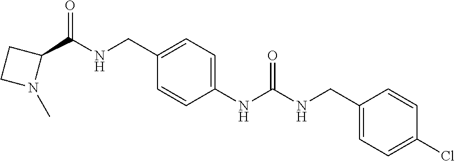 [SMILES: CN1CC[C@H]1C(=O)NCc1ccc(NC(=O)NCc2ccc(Cl)cc2)cc1] |
| | | | (S)-N-(4-(3-(4-chlorobenzyl)ureido)benzyl)-1- |
| | | | methylazetidine-2-carboxamide. |
| | | | LCMS-ESI (POS.) m/z: 387.1 (M + H)+. ¹H |
| | | | NMR (400 MHz, DMSO-d₆) δ 8.58 (s, 1H), 8.17 |
| | | | (s, 1H), 7.39 (d, J = 8.5 Hz, 2H), 7.36-7.27 (m, |
| | | | 4H), 7.11 (d, J = 8.3 Hz, 2H), 6.66 (t, J = 6.1 Hz, |
| | | | 1H), 4.27 (d, J = 5.9 Hz, 2H), 4.20 (t, J = 6.8 Hz, |
| | | | 2H), 3.37 (t, J = 8.4 Hz, 1H), 3.29-3.22 (m, 1H), |
| | | | 2.89-2.80 (m, 1H), 2.25 (s, 3H), 2.18 (td, J = 9.3, |
| | | | 8.2, 2.3 Hz, 1H), 1.94 (dt, J = 18.4, 8.7 Hz, 1H). |

### Example 4

### Synthesis of 1-(4-((4-(2-hydroxy-2-methylpropyl)piperazin-1-yl)methyl)phenyl)-3-(4-methoxybenzyl)urea (Compound 242)

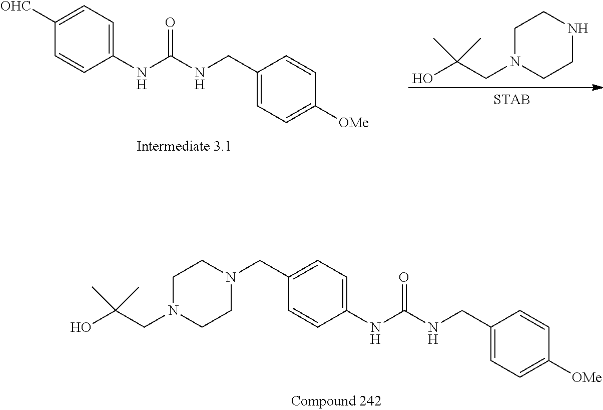 [SMILES: COc1ccc(CNC(=O)Nc2ccc(C=O)cc2)cc1 ; CC(C)(O)CN1CCNCC1 ; COc1ccc(CNC(=O)Nc2ccc(CN3CCN(CC(C)(C)O)CC3)cc2)cc1]

To a solution of amine and Intermediate 3.1 (100 mg, 0.35 mmol) and 2-methyl-1-(piperazin-1-yl)propan-2-ol (82 mg, 0.52 mmol) in DCE (2 mL) and pyridine (0.2 mL), preheated at 70° C. for 15 mins and subsequently cooled to room temperature, was added sodium triacetoxyborohydride (112 mg, 0.52 mmol) and the solution was stirred at 50 C for 12 h. The solution was cooled to room temperature and saturated aqueous sodium carbonate solution (3.0 mL) was added and the solution stirred vigorously for 10 mins. The organic layer was separated and the aqueous layer was extracted with 5 mL of DCM. The combined organic layer was washed with brine, dried, filtered, and concentrated. The crude was purified by reverse phase HPLC with a 10%-100% acetonitrile in water solution that was run over 30 minutes in a Phenomonex Gemini 5u C18 column, providing 1-(4-((4-(2-hydroxy-2-methylpropyl)piperazin-1-yl)methyl)phenyl)-3-(4-methoxybenzyl)urea (82 mg, 0.19 mmol) as a viscous pale yellow oil. LCMS-APCI (POS.) m/z: 427.2 (M+H)⁺. ¹H NMR (400 MHz, DMSO-d₆) δ 8.47 (s, 1H), 7.33 (d, J=8.1 Hz, 2H), 7.23 (d, J=8.1 Hz, 2H), 7.12 (d, J=8.0 Hz, 2H), 6.90 (d, J=8.2 Hz, 2H), 6.50 (t, J=5.9 Hz, 2H), 4.22 (d, J=5.8 Hz, 2H), 3.73 (s, 3H), 3.30 (s, 5H), 2.34 (s, 4H), 2.18 (s, 2H), 1.07 (s, 6H).

Compounds in the following table were prepared in a similar manner as Compound 242, using the intermediates and reagents as listed.

| Ex | | | |
|---|---|---|---|
| # | Intermediate | Reagent | Structure, Name and Data |
| 318 | 3.1 | morpholine | 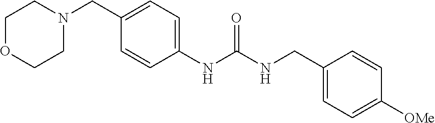 [SMILES: COc1ccc(CNC(=O)Nc2ccc(CN3CCOCC3)cc2)cc1] |
| | | | Compound 318: N-[(4- |
| | | | methoxyphenyl)methyl/{[4-(morpholin-4- |
| | | | ylmethyl)phenyl]amino}carboxamide. LCMS- |
| | | | ESI (POS.) m/z: 356.20 (M + H)+. ¹H NMR (400 |
| | | | MHz, DMSO-d₆) δ 8.53 (s, 1H), 7.34 (d, J = 8.1 |
| | | | Hz, 2H), 7.23 (d, J = 8.3 Hz, 3H), 7.14 (d, J = |
| | | | 8.1 Hz, 2H), 6.90 (d, J = 8.3 Hz, 2H), 6.56 (t, J = |
| | | | 5.9 Hz, 1H), 4.22 (d, J = 5.8 Hz, 2H), 3.74 (s, |
| | | | 3H), 3.56 (t, J = 4.6 Hz, 5H), 2.32 (t, J = 4.4 Hz, |
| | | | 4H). |
| 232 | 3.1 | 4,4-difluoro- piperidine | 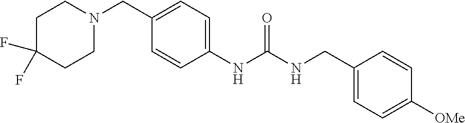 [SMILES: COc1ccc(CNC(=O)Nc2ccc(CN3CCC(F)(F)CC3)cc2)cc1] |
| | | | Compound 232: ({4-[(4,4- |
| | | | difluoropiperidyl)methyl]phenyl}amino)-N- |
| | | | [(4-methoxyphenyl)methyl]carboxamide. |
| | | | LCMS-ESI (POS.) m/z: 390.20 (M + H)+. ¹H |
| | | | NMR (400 MHz, DMSO-d₆) δ 7.36 (d, J = 8.1 |
| | | | Hz, 2H), 7.23 (d, J = 8.2 Hz, 2H), 7.15 (d, J = |
| | | | 8.0 Hz, 2H), 6.90 (d, J = 8.3 Hz, 2H), 6.63 (t, J = |
| | | | 5.9 Hz, 1H), 4.22 (d, J = 5.8 Hz, 2H), 3.74 (s, |
| | | | 3H), 3.45 (s, 2H), 1.94 (tt, J = 12.1, 5.5 Hz, 4H). |
| 257 | 3.1 | thiomorpholine 1,1-dioxide | 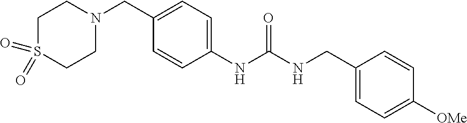 [SMILES: COc1ccc(CNC(=O)Nc2ccc(CN3CCS(=O)(=O)CC3)cc2)cc1] |
| | | | Compound 257: ({4-[(1,1-dioxo(l,4- |
| | | | thiazaperhydroin-4- |
| | | | yl))methyl] phenyl}amino)-N-[(4- |
| | | | methoxyphenyl)methyl]carboxamide. LCMS- |
| | | | ESI (POS.) m/z: 404.20 (M + H)+. ¹H NMR (400 |
| | | | MHz, DMSO-d₆) δ 7.37 (d, J = 8.0 Hz, 2H), |
| | | | 7.20 (dd, J = 21.9, 8.1 Hz, 4H), 6.90 (d, J = 8.3 |
| | | | Hz, 2H), 6.54 (t, J = 5.9 Hz, 1H), 4.22 (d, J = 5.7 |
| | | | Hz, 2H), 3.74 (s, 3H), 3.57 (s, 2H), 3.09 (t, J = |
| | | | 5.0 Hz, 4H), 2.84 (dd, J = 6.8, 3.5 Hz, 4H). |
| 264 | 3.1 | piperidine | 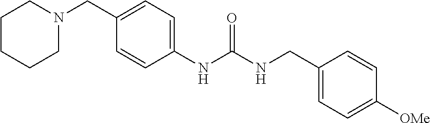 [SMILES: COc1ccc(CNC(=O)Nc2ccc(CN3CCCCC3)cc2)cc1] |
| | | | Compound 264: N-[(4- |
| | | | methoxyphenyl)methyl]{[4- |
| | | | (piperidylmethyl)phenyl]amino}carboxamide. |
| | | | LCMS-ESI (POS.) m/z: 353.21 (M + H)+. ¹H |
| | | | NMR (400 MHz, DMSO-d₆) δ 8.53 (s, 1H), 7.36 |
| | | | (d, J = 8.2 Hz, 2H), 7.23 (d, J = 8.2 Hz, 2H), |
| | | | 7.17 (d, J = 8.1 Hz, 2H), 6.90 (d, J = 8.3 Hz, |
| | | | 3H), 6.52 (d, J = 6.9 Hz, 1H), 4.22 (d, J = 5.8 |
| | | | Hz, 2H), 3.74 (s, 4H), 3.54 (d, J = 16.1 Hz, 2H), |
| | | | 2.34 (s, 2H), 1.71-1.51 (m, 4H), 1.45-1.41 |
| | | | (m, 2H). |
| 354 | 3.1 | (R)- pyrrolidin-3-ol | 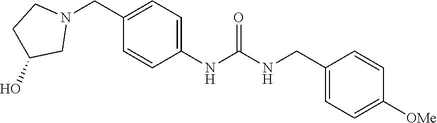 [SMILES: COc1ccc(CNC(=O)Nc2ccc(CN3CC[C@@H](O)C3)cc2)cc1] |
| | | | Compound 354: ({4-[((3R)-3- |
| | | | hydroxypyrrolidinyl)methyl]phenyl}amino)- |
| | | | N-[(4-methoxyphenyl)methyl]carboxamide. |
| | | | LCMS-ESI (POS.) m/z: 356.20 (M + H)+. ¹H |
| | | | NMR (400 MHz, DMSO-d₆) δ 8.46 (s, 1H), 7.33 |
| | | | (d, J = 8.0 Hz, 2H), 7.23 (d, J = 8.0 Hz, 3H), |
| | | | 7.15 (d, J = 8.1 Hz, 2H), 6.90 (d, J = 8.1 Hz, |
| | | | 3H), 6.49 (t, J = 6.0 Hz, 1H), 4.70 (d, J = 4.3 Hz, |
| | | | 1H), 4.21 (t, J = 8.1 Hz, 4H), 3.74 (s, 3H), 3.55- |
| | | | 3.43 (m, 2H), 2.67 (dd, J = 10.0, 6.1 Hz, 1H), |
| | | | 2.57 (d, J = 7.8 Hz, 1H), 2.42 (d, J = 6.9 Hz, |
| | | | 1H), 2.30 (dd, J = 9.5, 3.4 Hz, 1H), 1.99 (dq, J = |
| | | | 14.3, 7.4 Hz, 1H), 1.67-1.37 (m, 1H). |
| 322 | 3.1 | piperidin-4-ol | 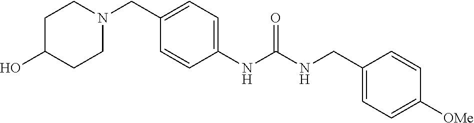 [SMILES: COc1ccc(CNC(=O)Nc2ccc(CN3CCC(O)CC3)cc2)cc1] |
| | | | Compound 322: N-{4-[(4- |
| | | | hydroxypiperidyl)methyl]phenyl}{[(4- |
| | | | methoxyphenyl)methyl]amino}carboxamide. |
| | | | LCMS-ESI (POS.) m/z: 370.20 (M + H)+. ¹H |
| | | | NMR (400 MHz, DMSO-d₆) δ 8.47 (s, 1H), 7.33 |
| | | | (d, J = 8.1 Hz, 2H), 7.23 (d, J = 8.1 Hz, 2H), |
| | | | 7.12 (d, J = 8.1 Hz, 2H), 6.90 (d, J = 8.2 Hz, |
| | | | 2H), 6.50 (t, J = 5.9 Hz, 1H), 4.53 (d, J = 4.1 Hz, |
| | | | 1H), 4.22 (d, J = 5.7 Hz, 2H), 3.74 (s, 3H), 3.52- |
| | | | 3.38 (m, 2H), 2.74-2.58 (m, 3H), 1.98 (t, J = |
| | | | 10.7 Hz, 2H), 1.73-1.60 (m, 3H), 1.48-1.28 |
| | | | (m, 2H). |
| 288 | 3.1 | 4-fluoro- piperidine | 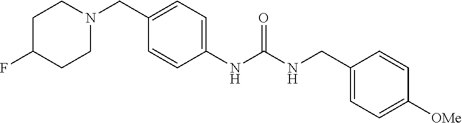 [SMILES: COc1ccc(CNC(=O)Nc2ccc(CN3CCC(F)CC3)cc2)cc1] |
| | | | Compound 288: N-{4-[(4- |
| | | | fluoropiperidyl)methyl] phenyl}{[(4- |
| | | | methoxyphenyl)methyl]amino}carboxamide. |
| | | | LCMS-ESI (POS.) m/z: 372.20 (M + H)+. ¹H |
| | | | NMR (400 MHz, DMSO-d₆) δ 8.53 (s, 1H), 7.36 |
| | | | (d, J = 8.2 Hz, 2H), 7.23 (d, J = 8.2 Hz, 2H), |
| | | | 7.17 (d, J = 8.1 Hz, 2H), 6.90 (d, J = 8.3 Hz, |
| | | | 3H), 6.52 (d, J = 6.9 Hz, 1H), 6.90 (dq, J = 25.2, |
| | | | 5.6 Hz, 3H), 4.22 (d, J = 5.8 Hz, 2H), 3.74 (s, |
| | | | 4H), 3.54 (d, J = 16.1 Hz, 2H), 2.34 (s, 2H), 1.71- |
| | | | 1.51 (m, 4H), 1.45-1.41 (m, 2H). |
| 351 | 3.1 | (R)-pyrrolidin- 3-ylmethanol | 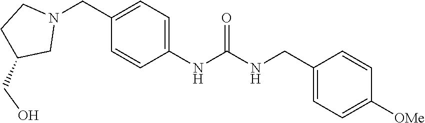 [SMILES: COc1ccc(CNC(=O)Nc2ccc(CN3CC[C@@H](CO)C3)cc2)cc1] |
| | | | Compound 351: N-(4-{[(3R)-3- |
| | | | (hydroxymethyl)pyrrolidinyl]methyl}phenyl) |
| | | | {1(4- |
| | | | methoxyphenyl)methyl]amino}carboxamide. |
| | | | LCMS-ESI (POS.) m/z: 372.20 (M + H)+. ¹H |
| | | | NMR (400 MHz, DMSO-d₆) δ 8.59 (s, 1H), 7.35 |
| | | | (d, J = 8.0 Hz, 2H), 7.20 (dd, J = 12.4, 8.1 Hz, |
| | | | 5H), 6.89 (d, J = 7.9 Hz, 3H), 6.60 (t, J = 6.1 Hz, |
| | | | 1H), 4.21 (d, J = 5.8 Hz, 3H), 3.72 (s, 4H), 3.30 |
| | | | (p, J = 10.2 Hz, 2H), 2.69 (t, J = 8.8 Hz, 1H), |
| | | | 2.60 (s, 0H), 2.38 (dd, J = 9.8, 6.4 Hz, 1H), 2.25 |
| | | | (p, J = 7.2 Hz, 1H), 1.84 (dq, J = 15.4, 7.4 Hz, |
| | | | 1H), 1.43 (dq, J = 13.4, 6.9 Hz, 1H). |
| 276 | 3.1 | morpholin-2- ylmethanol | 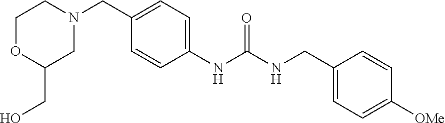 [SMILES: COc1ccc(CNC(=O)Nc2ccc(CN3CCOC(CO)C3)cc2)cc1] |
| | | | Compound 276: N-(4-{[2- |
| | | | (hydroxymethyl)morpholin-4- |
| | | | yl]methyl}phenyl){[(4- |
| | | | methoxyphenyl)methyl]amino}carboxamide. |
| | | | LCMS-ESI (POS.) m/z: 386.20 (M + H)+. ¹H |
| | | | NMR (400 MHz, DMSO-d₆) δ 8.47 (s, 1H), 7.34 |
| | | | (d, J = 8.1 Hz, 2H), 7.23 (d, J = 8.3 Hz, 2H), |
| | | | 7.14 (d, J = 8.1 Hz, 2H), 6.90 (d, J = 8.4 Hz, |
| | | | 2H), 6.50 (t, J = 5.9 Hz, 1H), 4.61 (t, J = 5.5 Hz, |
| | | | 1H), 4.22 (d, J = 5.8 Hz, 2H), 3.74 (s, 4H), 3.26 |
| | | | (q, J = 6.7, 5.7 Hz, 1H), 2.73 (d, J = 11.2 Hz, |
| | | | 1H), 3.25-3.17 (m, 5H), 2.60 (d, J = 11.4 Hz, |
| | | | 1H), 2.01 (td, J = 11.4, 3.3 Hz, 1H), 1.73 (d, J = |
| | | | 10.2 Hz, 1H). |
| 201 | 3.1 | 2- (trifluoromethyl) piperazine | 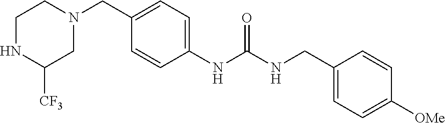 [SMILES: COc1ccc(CNC(=O)Nc2ccc(CN3CCNC(C(F)(F)F)C3)cc2)cc1] |
| | | | Compound 201: {1(4- |
| | | | methoxyphenyl)methyl]amino}-N-(4-{[3- |
| | | | (trifluoromethyl)piperazinyl]methyl}phenyl) |
| | | | carboxamide. LCMS-ESI (POS.) m/z: 423.20 |
| | | | (M + H)+. ¹H NMR (400 MHz, DMSO-d₆) δ 8.51 |
| | | | (s, 1H), 7.35 (d, J = 7.9 Hz, 2H), 7.23 (d, J = 8.1 |
| | | | Hz, 2H), 7.14 (d, J = 7.9 Hz, 2H), 6.97-6.78 |
| | | | (m, 2H), 6.54 (t, J = 6.0 Hz, 1H), 4.22 (d, J = 5.7 |
| | | | Hz, 2H), 3.8-3.3 (br s, 4H), 3.74 (s, 2H), 3.31 (t, |
| | | | J = 8.3Hz, 1H), 2.86 (d, J = 11.9 Hz, 1H), 2.76 |
| | | | (d, J = 10.7 Hz, 1H), 2.67 (t, J = 11.3 Hz, 1H), |
| | | | 2.60 (d, J = 11.2 Hz, 1H), 1.98 (dt, J = 19.7, 10.4 |
| | | | Hz, 2H). |
| 259 | 3.1 | 1-(pyridin-2- ylmethyl) piperazine | 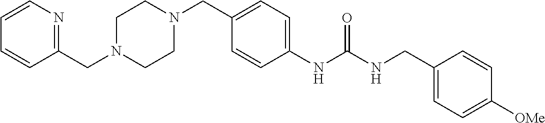 [SMILES: COc1ccc(CNC(=O)Nc2ccc(CN3CCN(Cc4ccccn4)CC3)cc2)cc1] |
| | | | Compound 259: {[(4- |
| | | | methoxyphenyl)methyl] amino}-N-(4-{[4-(2- |
| | | | pyridylmethyl)piperazinyl]methyl}phenyl)car |
| | | | boxamide. LCMS-ESI (POS.) m/z: 446.20 |
| | | | (M + H)+. ¹H NMR (400 MHz, DMSO-d₆) 8 8.54 |
| | | | (s, 1H), 8.48 (d, J = 4.7 Hz, 1H), 8.20 (s, 1H), |
| | | | 7.75 (t, J = 7.7 Hz, 1H), 7.42 (d, J = 7.9 Hz, 1H), |
| | | | 7.35 (d, J = 8.0 Hz, 2H), 7.23 (d, J = 8.0 Hz, |
| | | | 2H), 7.14 (d, J = 8.0 Hz, 2H), 6.89 (d, J = 8.0 |
| | | | Hz, 2H), 6.57 (t, J = 6.0 Hz, 1H), 4.8-4.2 (br s, |
| | | | 4H), 4.22 (d, J = 5.7 Hz, 2H), 3.73 (s, 3H), 3.59 |
| | | | (s, 2H), 2.43 (s, 6H). |
| 251 | 3.1 | 1-(oxetan- 3-yl) piperazine | 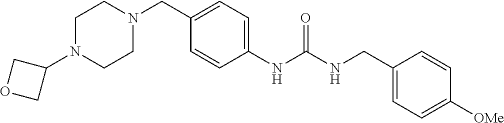 [SMILES: COc1ccc(CNC(=O)Nc2ccc(CN3CCN(C4COC4)CC3)cc2)cc1] |
| | | | Compound 251: {[(4- |
| | | | methoxyphenyl)methyl]amino}-N-{4-[(4- |
| | | | oxetan-3- |
| | | | ylpiperazinyl)methyl]phenyl}carboxamide. |
| | | | LCMS-ESI (POS.) m/z: 411.20 (M + H)+. ¹H |
| | | | NMR (400 MHz, DMSO-d₆) δ 8.51 (s, 1H), 7.34 |
| | | | (d, J = 7.8 Hz, 2H), 7.26-7.20 (m, 2H), 7.13 (d, |
| | | | J = 7.9 Hz, 2H), 6.96-6.86 (m, 2H), 6.54 (t, J = |
| | | | 5.9 Hz, 1H), 4.51 (t, J = 6.5 Hz, 2H), 4.3-3.5 (br |
| | | | s, 4H), 4.40 (t, J = 6.1 Hz, 2H), 4.22 (d, J = 5.7 |
| | | | Hz, 2H), 3.74 (s, 3H), 2.38 (s, 4H), 2.25 (s, 3H). |
| 307 | 3.1 | 1-((1H- pyrazol-4-yl) methyl) piperazine | 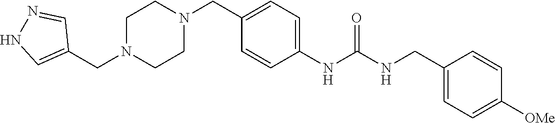 [SMILES: COc1ccc(CNC(=O)Nc2ccc(CN3CCN(Cc4cn[nH]c4)CC3)cc2)cc1] |
| | | | Compound 307: {[(4- |
| | | | methoxyphenyl)methyl]amino}-N-(4-{[4- |
| | | | (pyrazol-4-ylmethyl) |
| | | | piperazinyl]methyl}phenyl)carboxamide. |
| | | | LCMS-ESI (POS.) m/z: 435.20 (M + H)+. |
| | | | ¹H NMR (400 MHz, DMSO-d₆) δ 8.55 (s, 1H), |
| | | | 8.23 (s, 1H), 7.46 (s, 2H), 7.33 (d, J = 7.9 Hz, |
| | | | 2H), 7.23 (d, J = 8.0 Hz, 2H), 7.11 (d, J = 8.0 |
| | | | Hz, 2H), 6.90 (d, J = 8.0 Hz, 2H), 6.58 (t, J = 6.0 |
| | | | Hz, 1H), 4.21 (d, J = 5.7 Hz, 2H), 3.91 (s, 1H), |
| | | | 3.74 (s, 3H), 3.23 (d, J = 7.6 Hz, 1H), 3.18 (s, |
| | | | 1H), 3.02 (d, J = 7.4 Hz, 1H), 2.34 (s, 8H). |
| 267 | 3.1 | 1-isobutyl piperazine | 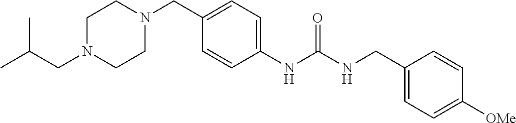 [SMILES: COc1ccc(CNC(=O)Nc2ccc(CN3CCN(CC(C)C)CC3)cc2)cc1] |
| | | | Compound 267: {[(4- |
| | | | methoxyphenyl)methyl]amino}-N-(4-{[4-(2- |
| | | | methylpropyl)piperazinyl]methyl}phenyl) |
| | | | carboxamide. LCMS-ESI (POS.) m/z: 411.20 |
| | | | (M + H)+. ¹H NMR (400 MHz, DMSO-d₆) δ 8.52 |
| | | | (s, 1H), 7.34 (d, J = 8.0 Hz, 2H), 7.23 (d, J = 8.1 |
| | | | Hz, 2H), 7.13 (d, J = 8.0 Hz, 2H), 6.95-6.83 |
| | | | (m, 2H), 6.55 (s, 1H), 4.22 (d, J = 5.7 Hz, 2H), |
| | | | 4.15-3.65 (br s, 2H), 3.74 (d, J = 1.7 Hz, 3H), |
| | | | 2.36 (s, 8H), 2.03 (d, J = 7.0 Hz, 2H), 1.74 (dt, J = |
| | | | 13.6, 6.8 Hz, 1H), 0.84 (dd, J = 6.4, 1.7 Hz, |
| | | | 6H). |
| 226 | 3.1 | 7,7-difluoro- octahydropyrrolo [1,2-a]pyrazine | 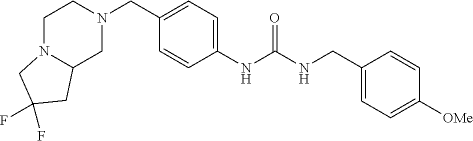 [SMILES: COc1ccc(CNC(=O)Nc2ccc(CN3CCN4CC(F)(F)CC4C3)cc2)cc1] |
| | | | Compound 226: (4-2-(3,3- |
| | | | difluoropyrrolidinyl)-2- |
| | | | oxoethyl] phenyl}amino)-N- [(4- |
| | | | methoxyphenyl)methyl]carboxamide. LCMS- |
| | | | ESI (POS.) m/z: 411.20 (M + H)+. ¹H NMR (400 |
| | | | MHz, DMSO-d₆) δ 8.51 (s, 1H), 7.39-7.30 (m, |
| | | | 2H), 7.26-7.21 (m, 2H), 7.13 (d, J = 8.0 Hz, |
| | | | 2H), 6.94-6.83 (m, 2H), 6.54 (t, J = 5.9 Hz, |
| | | | 1H), 4.22 (d, J = 5.7 Hz, 2H), 3.74 (d, J = 1.7 |
| | | | Hz, 3H), 4.15-3.65 (br s, 2H), 3.60-3.15 (m, |
| | | | 3H), 2.72 (d, J = 10.9 Hz, 1H), 2.51 (d, J = 2.9 |
| | | | Hz, 1H), 2.39 (t, J = 8.7 Hz, 1H), 2.23 (dd, J = |
| | | | 15.8, 7.4 Hz, 2H), 2.13 (dd, J = 12.3, 9.7 Hz, |
| | | | 1H), 1.83 (h, J = 13.5 Hz, 2H). |
| 339 | 3.1 | 2-(piperazin- 1-yl)ethan- 1-ol | 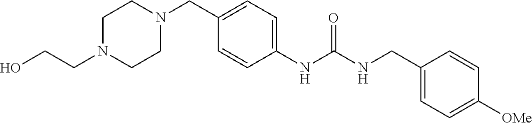 [SMILES: COc1ccc(CNC(=O)Nc2ccc(CN3CCN(CCO)CC3)cc2)cc1] |
| | | | Compound 339: 1-(4-((4-(2- |
| | | | hydroxyethyl)piperazin-1-yl)methyl)phenyl)- |
| | | | 3-(4-methoxybenzyl)urea. LCMS-ESI (POS.) |
| | | | m/z: 399.20 (M + H)+. ¹H NMR (400 MHz, |
| | | | DMSO-d₆) δ 8.59 (s, 1H), 8.23 (s, 1H), 7.36 (d, J = |
| | | | 7.9 Hz, 2H), 7.24 (d, J = 8.0 Hz, 2H), 7.14 (d, |
| | | | J = 8.0 Hz, 2H), 6.90 (d, J = 8.2 Hz, 2H), 6.62 (t, |
| | | | J = 6.2 Hz, 1H), 4.22 (d, J = 5.6 Hz, 2H), 3.74 |
| | | | (d, J = 1.7 Hz, 2H), 3.52 (d, J = 6.2 Hz, 2H), |
| | | | 3.30 (s, 5H), 2.54 (m, 4H), 2.45 (m, 4H). |
| 297 | 3.1 | 1-(2- methoxyethyl) piperazine | 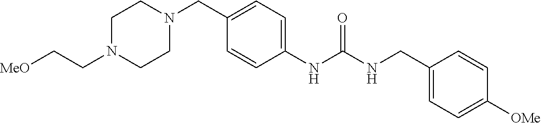 [SMILES: COCCN1CCN(Cc2ccc(NC(=O)NCc3ccc(OC)cc3)cc2)CC1] |
| | | | Compound 297: N-(4-{[4-(2- |
| | | | methoxyethyl)piperazinyl]methyl}phenyl){[(4- |
| | | | methoxyphenyl)methyl]amino}carboxamide. |
| | | | LCMS-ESI (POS.) m/z: 413.20 (M + H)+. ¹H |
| | | | NMR (400 MHz, DMSO-d₆) δ 8.52 (s, 1H), 7.38- |
| | | | 7.31 (m, 2H), 7.27-7.19 (m, 2H), 7.17-7.09 |
| | | | (m, 2H), 6.93-6.86 (m, 2H), 6.58 (t, J = 6.2 Hz, |
| | | | 1H), 4.22 (d, J = 5.7 Hz, 2H), 3.74 (s, 3H), 3.46- |
| | | | 3.35 (m, 4H), 3.23 (d, J = 1.7 Hz, 3H), 2.48 (d, |
| | | | J = 5.9 Hz, 2H), 2.44 (s, 4H), 2.37 (s, 4H). |
| 306 | 3.1 | 2-methyl-2- (piperazin-1- yl)propan- 1-ol | 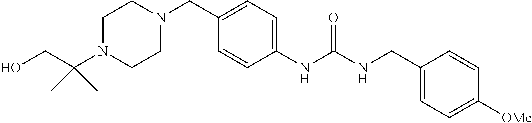 [SMILES: COc1ccc(CNC(=O)Nc2ccc(CN3CCN(C(C)(C)CO)CC3)cc2)cc1] |
| | | | Compound 306: N-(4-{[4-(2-hydroxy-tert- |
| | | | butyl)piperazinyl]methyl}phenyl){[(4- |
| | | | methoxyphenyl)methyl]amino}carboxamide. |
| | | | LCMS-ESI (POS.) m/z: 427.20 (M + H)+. ¹H |
| | | | NMR (400 MHz, DMSO-d₆) 8 8.50 (s, 1H), |
| | | | 7.39-7.31 (m, 2H), 7.27-7.19 (m, 2H), 7.17- |
| | | | 7.09 (m, 2H), 6.93-6.86 (m, 2H), 6.65 (t, J = |
| | | | 6.0 Hz, 1H), 4.55-4.10 (br s, 4H), 4.22 (d, J = |
| | | | 5.7 Hz, 2H), 3.74 (d, J = 1.8 Hz, 2H), 3.38 (s, |
| | | | 2H), 2.67 (s, 4H), 2.51 (d, J = 2.0 Hz, 2H), 2.41 |
| | | | (s, 2H), 0.99 (d, J = 1.8 Hz, 6H). |
| 263 | 3.1 | 1-neopentyl- piperazine | 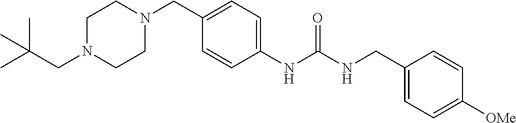 [SMILES: COc1ccc(CNC(=O)Nc2ccc(CN3CCN(CC(C)(C)C)CC3)cc2)cc1] |
| | | | Compound 263: N-(4-{[4-(2,2- |
| | | | dimethylpropyl)piperazinyl]methyl}phenyl) |
| | | | {[(4- |
| | | | methoxyphenyl)methyl]amino}carboxamide. |
| | | | LCMS-ESI (POS.) m/z: 425.20 (M + H)+. ¹H |
| | | | NMR (400 MHz, DMSO-d₆) δ 8.54 (s, 1H), 7.39- |
| | | | 7.31 (m, 2H), 7.27-7.19 (m, 2H), 7.18-7.11 |
| | | | (m, 2H), 6.93-6.86 (m, 2H), 6.57 (t, J = 6.0 Hz, |
| | | | 1H), 4.22 (d, J = 5.7 Hz, 2H), 3.74 (d, J = 1.6 |
| | | | Hz, 3H), 3.44 (s, 2H), 2.49 (t, J = 4.4 Hz, 4H), |
| | | | 2.41 (s, 4H), 2.04 (s, 2H), 0.83 (d, J = 1.7 Hz, |
| | | | 9H). |
| 317 | 3.1 | N-methyl-2- (piperazin-1- yl)acetamide | 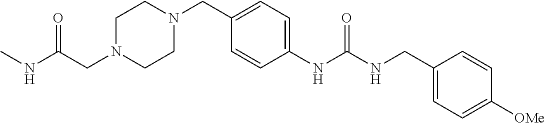 [SMILES: CNC(=O)CN1CCN(Cc2ccc(NC(=O)NCc3ccc(OC)cc3)cc2)CC1] |
| | | | Compound 317: 2-(4-{[4-({[(4- |
| | | | methoxyphenyl)methyl]amino}carbonylamino) |
| | | | phenyl]methyl}piperazinyl)-N- |
| | | | methylacetamide. LCMS-ESI (POS.) m/z: |
| | | | 426.20 (M + H)+. ¹H NMR (400 MHz, DMSO- |
| | | | d₆) δ 8.51 (s, 1H), 7.61 (q, J = 4.8 Hz, 1H), 7.35 |
| | | | (d, J = 7.9 Hz, 2H), 7.23 (d, J = 8.1 Hz, 2H), |
| | | | 7.14 (d, J = 8.0 Hz, 2H), 6.90 (d, J = 8.0 Hz, |
| | | | 2H), 6.54 (t, J = 6.0 Hz, 1H), 4.22 (d, J = 5.7 Hz, |
| | | | 2H), 3.74 (s, 3H), 3.42 (s, 2H), 2.88 (s, 2H), 2.61 |
| | | | (d, J = 4.6 Hz, 3H), 2.42 (s, 8H). |
| 243 | 3.1 | N,N- dimethyl-2- (piperazin-1- yl)acetamide | 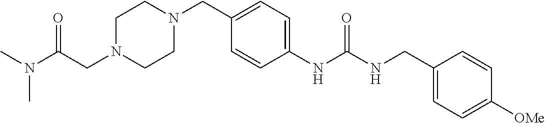 [SMILES: COc1ccc(CNC(=O)Nc2ccc(CN3CCN(CC(=O)N(C)C)CC3)cc2)cc1] |
| | | | Compound 243: 2-(4-{[4-({[(4- |
| | | | methoxyphenyl)methyl]amino}carbonylamino) |
| | | | phenyl]methyl}piperazinyl)-N,N- |
| | | | dimethylacetamide. LCMS-ESI (POS.) m/z: |
| | | | 440.20 (M + H)+. ¹H NMR (400 MHz, DMSO- |
| | | | d₆) δ 8.52 (s, 1H), 7.38-7.31 (m, 2H), 7.27- |
| | | | 7.19 (m, 2H), 7.13 (d, J = 7.9 Hz, 2H), 6.94- |
| | | | 6.86 (m, 2H), 6.55 (t, J = 5.9 Hz, 1H), 4.22 (d, J = |
| | | | 5.8 Hz, 2H), 3.74 (d, J = 1.7 Hz, 3H), 3.13 (s, |
| | | | 3H), 3.00 (d, J = 1.7 Hz, 3H), 2.80 (s, 3H), 2.45 |
| | | | (s, 4H), 2.38 (s, 5H). |
| 304 | 3.1 | 3-(piperazin- 1-yl) propanenitrile | 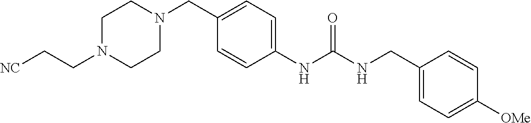 [SMILES: COc1ccc(CNC(=O)Nc2ccc(CN3CCN(CCC#N)CC3)cc2)cc1] |
| | | | Compound 304: N-(4-{[4-(2- |
| | | | cyanoethyl)piperazinyl]methyl}phenyl){[(4- |
| | | | methoxyphenyl)methyl]amino}carboxamide. |
| | | | LCMS-ESI (POS.) m/z: 408.20 (M + H)+. ¹H |
| | | | NMR (400 MHz, DMSO-d₆) δ 8.52 (s, 1H), 7.35 |
| | | | (d, J = 7.9 Hz, 2H), 7.23 (d, J = 8.0 Hz, 2H), |
| | | | 7.14 (d, J = 7.9 Hz, 2H), 6.93-6.86 (m, 2H), |
| | | | 6.54 (t, J = 6.0 Hz, 1H), 4.22 (d, J = 5.8 Hz, 2H), |
| | | | 3.73 (d, J = 1.6 Hz, 3H), 3.41 (s, 2H), 2.65 (t, J = |
| | | | 6.7 Hz, 2H), 2.53 (d, J = 13.4 Hz, 2H), 2.46- |
| | | | 2.37 (m, 8H). |
| 295 | 3.1 | (7R,8aR)- octahydro- pyrrolo[1,2-a] pyrazin-7-ol | 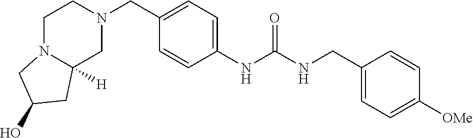 [SMILES: COc1ccc(CNC(=O)Nc2ccc(CN3CCN4C[C@H](O)C[C@@H]4C3)cc2)cc1] |
| | | | Compound 295: N-{4-[((lR,8R)-8-hydroxy- |
| | | | 3,6-diazabicyclo[4.3.0]non-3- |
| | | | yl)methyl] phenyl}{[(4- |
| | | | methoxyphenyl)methyl]amino}carboxamide. |
| | | | LCMS-ESI (POS.) m/z: 411.20 (M + H)+. ¹H |
| | | | NMR (400 MHz, DMSO-d₆) δ 8.47 (s, 1H), 7.34 |
| | | | (d, J = 8.1 Hz, 2H), 7.23 (d, J = 8.1 Hz, 2H), |
| | | | 7.14 (d, J = 8.0 Hz, 2H), 6.90 (d, J = 8.1 Hz, |
| | | | 2H), 6.55-6.39 (m, 1H), 4.76 (s, 1H), 4.21 (t, J = |
| | | | 9.4 Hz, 3H), 3.74 (s, 3H), 3.52-3.12 (m, 3H), |
| | | | 2.82 (t, J = 8.3 Hz, 2H), 2.70 (d, J = 10.8 Hz, |
| | | | 1H), 2.31 (q, J = 8.9, 8.2 Hz, 1H), 2.21 (t, J = |
| | | | 10.9 Hz, 1H), 2.08 (t, J = 11.1 Hz, 1H), 1.96 (t, J = |
| | | | 7.2 Hz, 1H), 1.71 (t, J = 10.2 Hz, 1H), 1.52 (t, |
| | | | J = 9.4 Hz, 2H). |
| 290 | 3.1 | 2-oxa-5,8- diazaspiro [3.5]nonane | 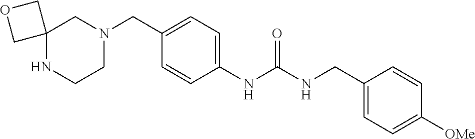 [SMILES: COc1ccc(CNC(=O)Nc2ccc(CN3CCNC4(COC4)C3)cc2)cc1] |
| | | | Compound 290: {1(4- |
| | | | methoxyphenyl)methyl]amino}-N-{4-[(2-oxa- |
| | | | 6,9-diazaspiro[3.5]non-6- |
| | | | yl)methyl]phenyl}carboxamide. LCMS-ESI |
| | | | (POS.) m/z: 397.20 (M + H)+. ¹H NMR (400 |
| | | | MHz, DMSO-d₆) δ 8.58 (t, J = 7.8 Hz, 1H), 8.48 |
| | | | (s, 1H), 8.21 (s, 1H), 7.69 (d, J = 6.2 Hz, 1H), |
| | | | 7.43 (d, J = 7.9 Hz, 2H), 7.23 (d, J = 8.1 Hz, |
| | | | 2H), 7.14 (d, J = 8.0 Hz, 2H), 6.88 (d, J = 8.0 |
| | | | Hz, 2H), 4.88 (d, J = 13.1 Hz, 1H), 4.52 (d, J = |
| | | | 13.1Hz, 1H), 4.20 (d, J = 5.4 Hz, 2H), 3.73 (s, |
| | | | 3H), 3.35-3.20 (m, 3H), 3.26 (d, J = 13.0 Hz, |
| | | | 1H), 3.15 (d, J = 11.1 Hz, 1H), 3.02 (t, J = 10.7 |
| | | | Hz, 1H), 2.93 (d, J = 11.1 Hz, 1H), 2.80 (d, J = |
| | | | 13.0 Hz, 1H), 2.46 (s, 1H), 2.35 (d, J = 11.2 Hz, |
| | | | 1H), 2.18- 1.89 (m, 2H). |
| 281 | 3.1 | 2-methyl-1- (3-methyl- piperazin-1- yl)propan- 2-ol | 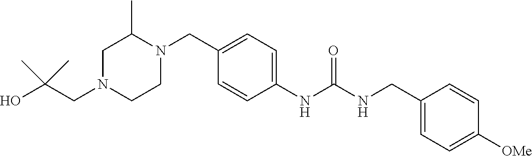 [SMILES: COc1ccc(CNC(=O)Nc2ccc(CN3CCN(CC(C)(C)O)CC3C)cc2)cc1] |
| | | | Compound 281: N-(4-{[4-(2-hydroxy-2- |
| | | | methylpropyl)-2- |
| | | | methylpiperazinyl]methyl}phenyl){[(4- |
| | | | methoxyphenyl)methyl]amino}carboxamide. |
| | | | LCMS-ESI (POS.) m/z: 441.20 (M + H)+. ¹H |
| | | | NMR (400 MHz, DMSO-d₆) δ 8.48 (s, 1H), 7.33 |
| | | | (d, J = 8.4 Hz, 2H), 7.22 (d, J = 8.5 Hz, 2H), |
| | | | 7.12 (d, J = 8.3 Hz, 2H), 6.89 (d, J = 8.5 Hz, |
| | | | 2H), 6.51 (t, J = 5.9 Hz, 1H), 4.21 (d, J = 5.8 Hz, |
| | | | 2H), 3.73 (s, 3H), 3.59 (s, 2H), 3.33 (s, 2H), 3.01 |
| | | | (dt, J = 12.2, 3.4 Hz, 1H), 2.48 (d, J = 5.1 Hz, |
| | | | 1H), 2.44 (s, 2H), 2.40-2.29 (m, 1H), 2.05 (d, J = |
| | | | 13.7 Hz, 1H), 1.93 (d, J = 9.8 Hz, 1H), 1.06 (d, |
| | | | J = 4.7 Hz, 6H), 0.93 (d, J = 6.1 Hz, 3H). |
| 340 | 3.1 | 2-methyl-1- (2-methyl- piperazin-1- yl)propan- 2-ol | 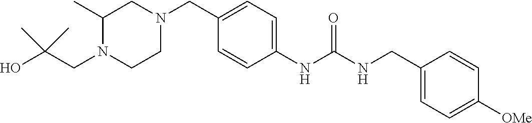 [SMILES: COc1ccc(CNC(=O)Nc2ccc(CN3CCN(CC(C)(C)O)C(C)C3)cc2)cc1] |
| | | | Compound 340: N-(4-{[4-(2-hydroxy-2- |
| | | | methylpropyl)-3- |
| | | | methylpiperazinyl]methyl}phenyl){[(4- |
| | | | methoxyphenyl)methyl]amino}carboxamide. |
| | | | LCMS-ESI (POS.) m/z: 441.20 (M + H)+. ¹H |
| | | | NMR (400 MHz, DMSO-d₆) δ 8.48 (s, 1H), 7.33 |
| | | | (d, J = 8.4 Hz, 2H), 7.23 (d, J = 8.7 Hz, 2H), |
| | | | 7.13 (d, J = 8.2 Hz, 2H), 6.93-6.86 (m, 2H), |
| | | | 6.51 (t, J = 5.9 Hz, 1H), 4.21 (d, J = 5.8 Hz, 2H), |
| | | | 3.85 (d, J = 13.2 Hz, 1H), 3.73 (s, 3H), 3.35- |
| | | | 3.30 (m, 1H), 3.09 (d, J = 13.2 Hz, 1H), 2.74 (d, |
| | | | J = 11.3 Hz, 1H), 2.65 (s, 1H), 2.42 (s, 1H), 2.28- |
| | | | 2.15 (m, 1H), 2.17-2.04 (m, 4H), 1.10-1.03 |
| 167 | 3.3 | 2-methyl-1- (piperazin-1- yl)propan- 2-ol | 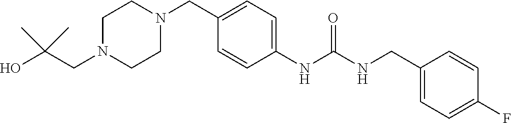 [SMILES: CC(C)(O)CN1CCN(Cc2ccc(NC(=O)NCc3ccc(F)cc3)cc2)CC1] |
| | | | Compound 167: {[(4- |
| | | | fluorophenyl)methyl]amino}-N-(4-{[4-(2- |
| | | | hydroxy-2-methylpropyl) |
| | | | piperazinyl]methyl}phenyl)carboxamide. |
| | | | LCMS-ESI (POS.) m/z: 415.20 |
| | | | (M + H)+. ¹H NMR (400 MHz, DMSO-d₆) δ 8.58 |
| | | | (s, 1H), 7.38-7.29 (m, 4H), 7.21-7.09 (m, |
| | | | 4H), 6.66 (t, J = 6.0 Hz, 1H), 4.27 (d, J = 5.9 Hz, |
| | | | 2H), 3.37 (s, 2H), 2.50-2.45 (m, 4H), 2.35 (s, |
| | | | 4H), 2.18 (s, 2H), 1.07 (s, 6H). |
| 36 | 3.2 | 2-methyl-1- (piperazin-1- yl)propan- 2-ol | 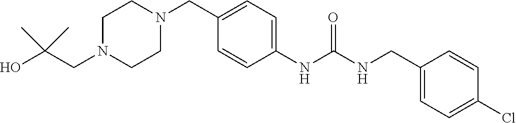 [SMILES: CC(C)(O)CN1CCN(Cc2ccc(NC(=O)NCc3ccc(Cl)cc3)cc2)CC1] |
| | | | Compound 67: {[(4- |
| | | | chlorophenyl)methyl] amino}-N-(4-{[4-(2- |
| | | | hydroxy-2-methylpropyl) |
| | | | piperazinyl]methyl}phenyl)carboxamide. |
| | | | LCMS-ESI (POS.) m/z: 431.20 |
| | | | (M + H)+. ¹H NMR (400 MHz, DMSO-d₆) δ 8.50 |
| | | | (s, 1H), 7.37-7.22 (m, 6H), 7.05 (d, J = 8.3 Hz, |
| | | | 2H), 6.58 (t, J = 6.1 Hz, 1H), 4.21 (d, J = 6.0 Hz, |
| | | | 2H), 3.96 (s, 1H), 2.50-2.45 (m, 6H), 2.36 (s, |
| | | | 1H), 2.27 (d, J = 9.9 Hz, 2H), 2.10 (s, 2H), 0.99 |
| | | | (s, 6H). |
| 333 | 3.1 | 2-methyl- 2,6- diazaspiro [3.3]heptane | 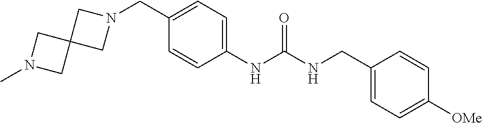 [SMILES: COc1ccc(CNC(=O)Nc2ccc(CN3CC4(CN(C)C4)C3)cc2)cc1] |
| | | | Compound 333: {[(4- |
| | | | methoxyphenyl)methyl]amino}-N-{4-[(6- |
| | | | methyl-2,6-diazaspiro[3.3]hept-2- |
| | | | yl)methyl]phenyl}carboxamide. LCMS-ESI |
| | | | (POS.) m/z: 381.20 (M + H)+. ¹H NMR (400 |
| | | | MHz, DMSO-d₆) δ 8.69 (s, 1H), 7.37-7.30 (m, |
| | | | 2H), 7.26-7.18 (m, 2H), 7.13-7.06 (m, 2H), |
| | | | 6.93-6.85 (m, 2H), 6.73 (t, J = 5.9 Hz, 1H), |
| | | | 4.35 (d, J = 28.7 Hz, 2H), 4.21 (d, J = 5.8 Hz, |
| | | | 2H), 3.73 (s, 3H), 3.57 (s, 4H), 3.27 (s, 4H), 2.39 |
| | | | (s, 3H). |
| 324 | 3.1 | azetidin-3- ylmethanol | 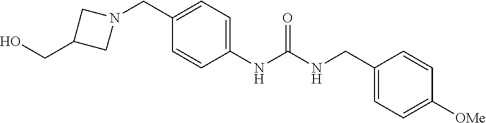 [SMILES: COc1ccc(CNC(=O)Nc2ccc(CN3CC(CO)C3)cc2)cc1] |
| | | | Compound 324: N-(4-{[3- |
| | | | (hydroxymethyl)azetidinyl]methyl}phenyl) |
| | | | {[(4-methoxyphenyl)methyl]amino}carboxamide. |
| | | | LCMS-ESI (POS.) m/z: 356.20 (M + H)+. ¹H |
| | | | NMR (400 MHz, DMSO-d₆) δ 8.61 (s, 1H), 7.29 |
| | | | (d, J = 8.4 Hz, 2H), 7.16 (d, J = 8.6 Hz, 2H), |
| | | | 7.08 (d, J = 8.2 Hz, 2H), 6.86-6.79 (m, 2H), |
| | | | 6.64 (t, J = 5.9 Hz, 1H), 4.14 (d, J = 5.8 Hz, 2H), |
| | | | 3.66 (s, 3H), 3.65-3.56 (m, 1H), 3.55 (s, 2H), |
| | | | 3.30 (t, J = 7.8 Hz, 2H), 3.02 (t, J = 7.1 Hz, 2H), |
| | | | 2.46 (dd, J = 16.0, 6.9 Hz, 2H). |
| 348 | 3.1 | 3-methyl- azetidin-3-ol | 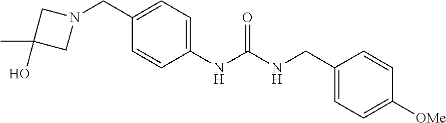 [SMILES: COc1ccc(CNC(=O)Nc2ccc(CN3CC(C)(O)C3)cc2)cc1] |
| | | | Compound 348: N-{4-[(3-hydroxy-3- |
| | | | methylazetidinyl)methyl]phenyl}{[(4- |
| | | | methoxyphenyl)methyl]amino}carboxamide. |
| | | | LCMS-ESI (POS.) m/z: 356.20 (M + H)+. ¹H |
| | | | NMR (400 MHz, DMSO-d₆) δ 8.51 (s, 1H), |
| | | | 7.33 (d, J = 8.0 Hz, 2H), 7.22 (d, J = 8.0 Hz, |
| | | | 2H), 7.12 (d, J = 8.0 Hz, 2H), 6.93-6.86 (m, |
| | | | 2H), 6.55 (t, J = 6.0 Hz, 1H), 5.23 (s, 1H), 4.21 |
| | | | (d, J = 5.7 Hz, 2H), 3.73 (s, 3H), 3.54 (s, 2H), |
| | | | 3.19 (d, J = 6.8 Hz, 2H), 2.94 (d, J = 6.8 Hz, |
| | | | 2H), 1.34 (s, 3H). |
| 282 | 3.1 | 7-methyl- 1,4- diazepan-5- one | 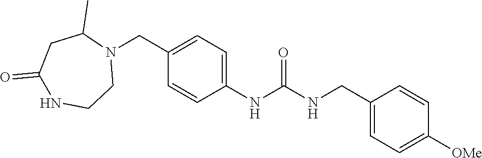 [SMILES: COc1ccc(CNC(=O)Nc2ccc(CN3CCNC(=O)CC3C)cc2)cc1] |
| | | | Compound 282: {[(4- |
| | | | methoxyphenyl)methyl]amino}-N-{4-[(7- |
| | | | methyl-5-oxo(1,4-diazaperhydroepinyl)) |
| | | | methyl]phenyl}carboxamide. |
| | | | LCMS-ESI (POS.) m/z: 383.20 (M + H)+. |
| | | | ¹H NMR (400 MHz, DMSO-d₆) δ 8.44 (s, 1H), |
| | | | 7.50 (s, 1H), 7.33 (d, J = 8.0 Hz, 2H), 7.20 (dd, J = |
| | | | 23.0, 8.3 Hz, 4H), 6.93-6.86 (m, 2H), 6.48 (t, |
| | | | J = 5.8 Hz, 1H), 4.21 (d, J = 5.8 Hz, 2H), 3.73 |
| | | | (s, 3H), 3.60 (d, J = 13.4 Hz, 1H), 3.52 (d, J = |
| | | | 13.7 Hz, 1H), 3.31 (s, 1H), 3.00 (s, 1H), 2.86 (d, |
| | | | J = 13.9 Hz, 2H), 2.67-2.56 (m, 2H), 2.20- |
| | | | 2.12 (m, 1H), 0.99 (d, J = 6.7 Hz, 3H). |
| 334 | 3.1 | 2-oxa-6- azaspiro[3.3] heptane | 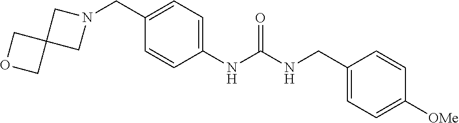 [SMILES: COc1ccc(CNC(=O)Nc2ccc(CN3CC4(COC4)C3)cc2)cc1] |
| | | | Compound 334: {[(4- |
| | | | methoxyphenyl)methyl]amino}-N-{4-[(6-oxa- |
| | | | 2-azaspiro[3.3]hept-2- |
| | | | yl)methyl]phenyl}carboxamide. LCMS-ESI |
| | | | (POS.) m/z: 368.20 (M + H)+. ¹H NMR (400 |
| | | | MHz, DMSO-d₆) δ 8.54 (s, 1H), 7.37-7.29 (m, |
| | | | 2H), 7.26-7.18 (m, 2H), 7.12 (d, J = 8.3 Hz, |
| | | | 2H), 6.94-6.85 (m, 2H), 6.57 (t, J = 5.9 Hz, |
| | | | 1H), 4.21 (d, J = 5.8 Hz, 2H), 3.73 (s, 3H), 3.54 |
| | | | (s, 2H), 3.47 (s, 4H), 3.00 (s, 4H). |
| 260 | 3.1 | 3-methyl- azetidine-3- carbonitrile | 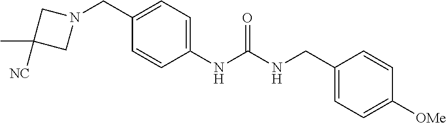 [SMILES: COc1ccc(CNC(=O)Nc2ccc(CN3CC(C)(C#N)C3)cc2)cc1] |
| | | | Compound 260: N-{4-[(3-cyano-3- |
| | | | methylazetidinyl)methyl]phenyl}{[(4- |
| | | | methoxyphenyl)methyl]amino}carboxamide. |
| | | | LCMS-ESI (POS.) m/z: 365.20 (M + H)+. ¹H |
| | | | NMR (400 MHz, DMSO-d₆) δ 8.48 (s, 1H), 7.37- |
| | | | 7.29 (m, 2H), 7.26-7.19 (m, 2H), 7.15-7.08 |
| | | | (m, 2H), 6.93-6.86 (m, 2H), 6.50 (t, J = 5.9 Hz, |
| | | | 1H), 4.21 (d, J = 5.8 Hz, 2H), 3.73 (s, 3H), 3.48 |
| | | | (s, 2H), 3.42 (d, J = 7.2 Hz, 2H), 3.09 (d, J = 7.2 |
| | | | Hz, 2H), 1.52 (s, 3H). |
| 265 | 3.1 | 3-(difluoro- methoxy) azetidine | 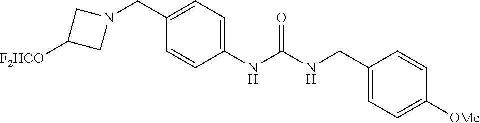 [SMILES: COc1ccc(CNC(=O)Nc2ccc(CN3CC(OC(F)F)C3)cc2)cc1] |
| | | | Compound 265: N-(4-{[3- |
| | | | (difluoromethoxy)azetidinyl]methyl}phenyl) |
| | | | {[(4-methoxyphenyl) |
| | | | methyl]amino}carboxamide. |
| | | | LCMS-ESI (POS.) m/z: 392.20 (M + H)+. ¹H |
| | | | NMR (400 MHz, DMSO-d₆) δ 8.48 (s, 1H), 7.33 |
| | | | (d, J = 8.5 Hz, 2H), 7.23 (d, J = 8.6 Hz, 2H), |
| | | | 7.11 (d, J = 8.5 Hz, 2H), 6.89 (d, J = 8.6 Hz, |
| | | | 2H), 6.66 (s, 1H), 6.51 (t, J = 5.9 Hz, 1H), 4.69 |
| | | | (t, J = 5.9 Hz, 1H), 4.21 (d, J = 5.8 Hz, 2H), 3.73 |
| | | | (s, 3H), 3.56-3.42 (m, 3H), 3.29 (t, J = 5.9 Hz, |
| | | | 1H), 2.98 (td, J = 5.9, 2.0 Hz, 2H). |
| 315 | 3.1 | (S)-1- (azetidin-3- yl)ethan-1-ol | 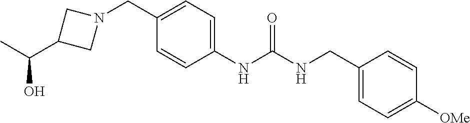 [SMILES: COc1ccc(CNC(=O)Nc2ccc(CN3CC([C@H](C)O)C3)cc2)cc1] |
| | | | Compound 265: N-(4-{[3-((1S)-1- |
| | | | hydroxyethyl)azetidinyl]methyl}phenyl){[(4- |
| | | | methoxyphenyl)methyl]amino}carboxamide. |
| | | | LCMS-ESI (POS.) m/z: 370.20 (M + H)+. ¹H |
| | | | NMR (400 MHz, DMSO-d₆) δ 8.56 (s, 1H), 7.33 |
| | | | (d, J = 8.4 Hz, 2H), 7.26-7.19 (m, 2H), 7.12 (d, |
| | | | J = 8.3 Hz, 2H), 6.94-6.85 (m, 2H), 6.60 (t, J = |
| | | | 5.9 Hz, 1H), 4.21 (d, J = 5.9 Hz, 2H), 3.73 (s, |
| | | | 5H), 3.51 (s, 2H), 3.26 (dt, J = 11.3, 7.5 Hz, 2H), |
| | | | 3.05 (t, J = 7.0 Hz, 1H), 2.90 (t, J = 7.1 Hz, 1H), |
| | | | 2.28 (p, J = 7.2 Hz, 1H), 0.95 (d, J = 6.2 Hz, |
| | | | 3H). |
| 246 | 3.1 | 3-(methyl- sulfonyl) azetidine | 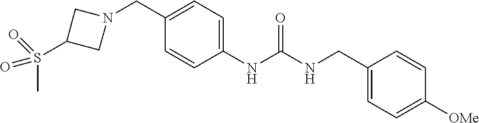 [SMILES: COc1ccc(CNC(=O)Nc2ccc(CN3CC(S(C)(=O)=O)C3)cc2)cc1] |
| | | | Compound 246: {[(4- |
| | | | methoxyphenyl)methyl]amino}-N-(4-{[3- |
| | | | (methylsulfonyl)azetidinyl]methyl}phenyl) |
| | | | carboxamide. LCMS-ESI (POS.) m/z: 404.20 |
| | | | (M + H)+. ¹H NMR (400 MHz, DMSO-d₆) δ 8.47 |
| | | | (s, 1H), 7.37-7.29 (m, 2H), 7.27-7.19 (m, |
| | | | 2H), 7.15-7.08 (m, 2H), 6.94-6.85 (m, 2H), |
| | | | 6.50 (t, J = 5.9 Hz, 1H), 4.21 (d, J = 5.8 Hz, 2H), |
| | | | 4.18-4.09 (m, 1H), 3.73 (s, 3H), 3.52-3.41 |
| | | | (m, 5H), 3.33 (d, J = 15.0 Hz, 1H), 2.95 (s, 3H). |
| 327 | 3.1 | 1-isopropyl- azetidin-3- amine | 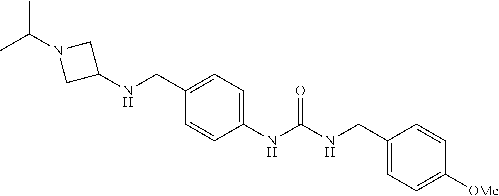 [SMILES: COc1ccc(CNC(=O)Nc2ccc(CNC3CN(C(C)C)C3)cc2)cc1] |
| | | | Compound 327: {[(4- |
| | | | methoxyphenyl)methyl]amino}-N-[4-({[1- |
| | | | (methylethyl)azetidin-3- |
| | | | yl]amino}methyl)phenyl]carboxamide. |
| | | | LCMS-ESI (POS.) m/z: 383.20 (M + H)+. ¹H |
| | | | NMR (400 MHz, DMSO-d₆) δ 8.60 (s, 1H), 8.29 |
| | | | (s, 1H), 7.39-7.30 (m, 2H), 7.26-7.19 (m, |
| | | | 2H), 7.15 (d, J = 8.2 Hz, 2H), 6.94-6.85 (m, |
| | | | 2H), 6.66 (t, J = 5.8 Hz, 1H), 4.21 (d, J = 5.9 Hz, |
| | | | 2H), 3.73 (s, 3H), 3.57 (d, J = 2.0 Hz, 1H), 3.28 |
| | | | (p, J = 6.7 Hz, 1H), 3.20-3.15 (m, 4H), 2.90 |
| | | | (dd, J = 8.5, 6.6 Hz, 2H), 0.90 (d, J = 6.2 Hz, |
| | | | 6H). |
| 200 | 3.1 | 2-(pyrrolidin- 2-yl)pyridine | 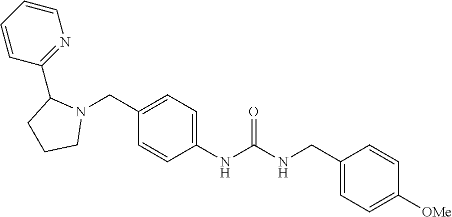 [SMILES: COc1ccc(CNC(=O)Nc2ccc(CN3CCCC3c3ccccn3)cc2)cc1] |
| | | | Compound 200: {[(4- |
| | | | methoxyphenyl)methyl]amino}-N-{4-[(2-(2- |
| | | | pyridyl)pyrrolidinyl)methyl]phenyl}carboxamide. |
| | | | LCMS-ESI (POS.) m/z: 417.20 (M + H)+. |
| | | | ¹H NMR (400 MHz, DMSO-d₆) δ 8.48 (dd, J = |
| | | | 7.4, 2.1 Hz, 2H), 7.80 (td, J = 7.7, 1.8 Hz, 1H), |
| | | | 7.62 (d, J = 7.9 Hz, 1H), 7.35-7.18 (m, 5H), |
| | | | 7.11 (d, J = 8.4 Hz, 2H), 6.94-6.85 (m, 2H), |
| | | | 6.50 (t, J = 5.9 Hz, 1H), 4.21 (d, J = 5.8 Hz, 2H), |
| | | | 3.73 (s, 3H), 3.67-3.49 (m, 2H), 3.10 (d, J = |
| | | | 13.0 Hz, 1H), 2.96 (ddd, J = 9.7, 6.8, 3.5 Hz, |
| | | | 1H), 2.30-2.14 (m, 2H), 1.84- 1.60 (m, 3H). |
| 300 | 3.1 | 4-methoxy-4- methyl- piperidine | 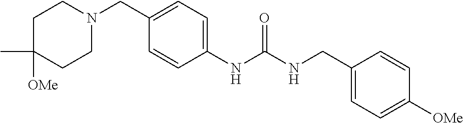 [SMILES: COc1ccc(CNC(=O)Nc2ccc(CN3CCC(C)(OC)CC3)cc2)cc1] |
| | | | Compound 300: N-{4-[(4-methoxy-4- |
| | | | methylpiperidyl)methyl] phenyl}{[(4- |
| | | | methoxyphenyl)methyl]amino}carboxamide. |
| | | | LCMS-ESI (POS.) m/z: 398.20 (M + H)+. ¹H |
| | | | NMR (400 MHz, DMSO-d₆) δ 8.51 (s, 1H), 7.38- |
| | | | 7.31 (m, 2H), 7.27-7.18 (m, 2H), 7.18-7.11 |
| | | | (m, 2H), 6.94-6.85 (m, 2H), 6.54 (t, J = 5.9 Hz, |
| | | | 1H), 4.22 (d, J = 5.8 Hz, 2H), 3.73 (s, 3H), 3.45- |
| | | | 3.32 (m, 2H), 3.04 (s, 3H), 2.29 (d, J = 14.2 |
| | | | Hz, 3H), 2.12 (d, J = 11.3 Hz, 1H), 1.64 (tt, J = |
| | | | 9.6, 6.6, 5.0 Hz, 1H), 1.42 (dq, J = 10.3, 6.2, 4.9 |
| | | | Hz, 3H), 1.08 (s, 3H). |
| 273 | 3.1 | 1-phenyl- cyclopropan-1- amine | 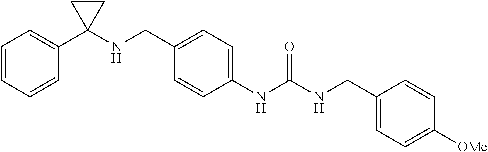 [SMILES: COc1ccc(CNC(=O)Nc2ccc(CNC3(c4ccccc4)CC3)cc2)cc1] |
| | | | Compound 273: {[(4- |
| | | | methoxyphenyl)methyl]amino}-N-(4- |
| | | | {[(phenylcyclopropyl)amino]methyl}phenyl) |
| | | | carboxamide. LCMS-ESI (POS.) m/z: 402.20 |
| | | | (M + H)+. ¹H NMR (400 MHz, DMSO-d₆) δ 8.46 |
| | | | (s, 1H), 8.17 (s, 1H), 7.43-7.27 (m, 6H), 7.27- |
| | | | 7.10 (m, 5H), 6.93-6.86 (m, 2H), 6.50 (t, J = |
| | | | 5.9 Hz, 1H), 4.21 (d, J = 5.8 Hz, 2H), 3.73 (s, |
| | | | 3H), 3.52 (s, 2H), 1.00 (q, J = 4.2 Hz, 2H), 0.89 |
| | | | (q, J = 4.3 Hz, 2H). |
| 332 | 3.1 | 2-azaspiro[3.3] heptan-6-ol | 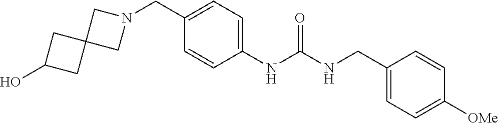 [SMILES: COc1ccc(CNC(=O)Nc2ccc(CN3CC4(CC(O)C4)C3)cc2)cc1] |
| | | | Compound 332: N-{4-[(6-hydroxy-2- |
| | | | azaspiro[3.3]hept-2-yl)methyl]phenyl}{[(4- |
| | | | methoxyphenyl)methyl]amino}carboxamide. |
| | | | LCMS-ESI (POS.) m/z: 382.20 (M + H)+. ¹H |
| | | | NMR (400 MHz, DMSO-d₆) δ 8.72 (s, 1H), 7.36 |
| | | | (d, J = 8.2 Hz, 2H), 7.23 (d, J = 8.5 Hz, 2H), |
| | | | 7.14 (d, J = 8.2 Hz, 2H), 6.94-6.85 (m, 2H), |
| | | | 6.73 (q, J = 13.6, 9.7 Hz, 1H), 4.35-4.10 (br s, |
| | | | 5H), 4.21 (d, J = 5.8 Hz, 2H), 3.93 (p, J = 7.3 |
| | | | Hz, 1H), 3.72 (d, J = 5.6 Hz, 1H), 3.62 (s, 2H), |
| | | | 3.31 (d, J = 19.2 Hz, 2H), 2.35 (ddd, J = 9.7, 6.9, |
| | | | 3.1 Hz, 2H), 1.94-1.84 (m, 2H). |
| 238 | 3.1 | 2-methyl-6- (pyrrolidin- 2-yl)pyridine | 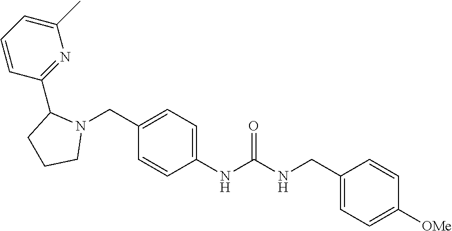 [SMILES: COc1ccc(CNC(=O)Nc2ccc(CN3CCCC3c3cccc(C)n3)cc2)cc1] |
| | | | Compound 238: {[(4- |
| | | | methoxyphenyl)methyl]amino}-N-(4-{[2-(6- |
| | | | methyl(2- |
| | | | pyridyl))pyrrolidinyl]methyl}phenyl)carboxamide. |
| | | | LCMS-ESI (POS.) m/z: 431.20 (M + H)+. |
| | | | ¹H NMR (400 MHz, DMSO-d₆) δ 8.61 (s, 1H), |
| | | | 7.36 (d, J = 8.4 Hz, 2H), 7.27-7.13 (m, 4H), |
| | | | 6.90 (d, J = 8.6 Hz, 2H), 6.64 (t, J = 6.0 Hz, 1H), |
| | | | 4.22 (d, J = 5.8 Hz, 2H), 3.95 (d, J = 12.9 Hz, |
| | | | 1H), 3.21 (d, J = 12.9 Hz, 1H), 2.83 (dt, J = 9.8, |
| | | | 5.6 Hz, 1H), 2.21 (q, J = 8.9 Hz, 1H), 1.95 (dq, J = |
| | | | 12.8, 7.3 Hz, 1H), 1.69-1.57 (m, 2H), 1.38 |
| | | | (dq, J = 12.3, 8.4 Hz, 1H), 1.12 (d, J = 6.1 Hz, |
| | | | 3H). |
| 337 | 3.1 | 6-oxa-1- azaspiro[3.3] heptane | 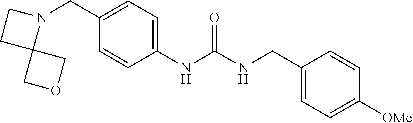 [SMILES: COc1ccc(CNC(=O)Nc2ccc(CN3CCC34COC4)cc2)cc1] |
| | | | Compound 337: {[(4- |
| | | | methoxyphenyl)methyl]amino}-N-{4-[(6-oxa-1- |
| | | | azaspiro[3.3]heptyl)methyl]phenyl}carboxamide. |
| | | | LCMS-ESI (POS.) m/z: 368.20 (M + H)+. ¹H |
| | | | NMR (400 MHz, DMSO-d₆) δ 8.48 (s, 1H), 7.28- |
| | | | 7.15 (m, 6H), 6.85 (d, J = 8.4 Hz, 2H), 6.61 (t, |
| | | | J = 6.0 Hz, 1H), 4.86 (d, J = 7.8 Hz, 2H), 4.32 |
| | | | (s, 2H), 4.30-4.07 (m, 2H), 3.75 (s, 2H), 3.67 |
| | | | (s, 3H), 3.01 (t, J = 7.1 Hz, 2H), 2.29 (t, J = 7.2 |
| | | | Hz, 2H). |
| 207 | 3.1 | 2-(2- chlorophenyl) pyrrolidine | 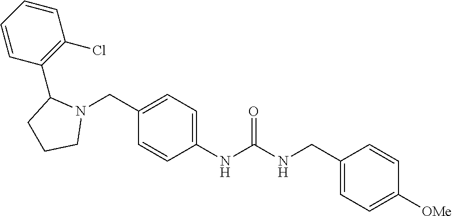 [SMILES: COc1ccc(CNC(=O)Nc2ccc(CN3CCCC3c3ccccc3Cl)cc2)cc1] |
| | | | Compound 207: N-(4-{[2-(3- |
| | | | chlorophenyl)pyrrolidinyl]methyl}phenyl){[(4- |
| | | | methoxyphenyl)methyl]amino}carboxamide. |
| | | | LCMS-ESI (POS.) m/z: 450.20 (M + H)+. ¹H |
| | | | NMR (400 MHz, DMSO-d₆) δ 8.50 (s, 1H), 7.81 |
| | | | (d, J = 7.7 Hz, 1H), 7.46-7.19 (m, 7H), 7.13 (d, |
| | | | J = 7.9 Hz, 2H), 6.90 (d, J = 7.9 Hz, 2H), 6.52 (t, |
| | | | J = 5.9 Hz, 1H), 4.21 (d, J = 5.1 Hz, 2H), 3.81 (t, |
| | | | J = 8.1 Hz, 1H), 3.73 (s, 3H), 3.64 (d, J = 12.9 |
| | | | Hz, 1H), 3.09-2.93 (m, 2H), 2.27 (dq, J = 35.1, |
| | | | 8.9, 8.3 Hz, 2H), 1.75 (p, J = 7.5 Hz, 2H), 1.44 |
| | | | (dq, J = 15.6, 8.2 Hz, 1H). |
| 291 | 3.1 | 2-(3-chloro- phenyl) pyrrolidine | 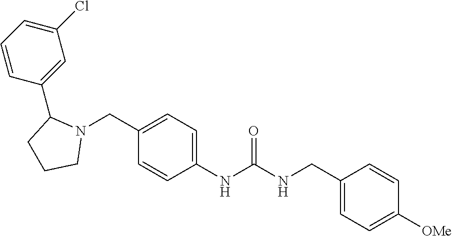 [SMILES: COc1ccc(CNC(=O)Nc2ccc(CN3CCCC3c3cccc(Cl)c3)cc2)cc1] |
| | | | Compound 291: N-(4-{2-(3- |
| | | | chlorophenyl)pyrrolidinyl]methyl}phenyl){[(4- |
| | | | methoxyphenyl)methyl]amino}carboxamide. |
| | | | LCMS-ESI (POS.) m/z: 450.20 (M + H)+. ¹H |
| | | | NMR (400 MHz, DMSO-d₆) δ 8.45 (s, 1H), 7.48 |
| | | | (s, 1H), 7.45-7.28 (m, 5H), 7.22 (d, J = 8.0 Hz, |
| | | | 2H), 7.09 (d, J = 7.8 Hz, 2H), 6.93-6.86 (m, |
| | | | 2H), 6.48 (t, J = 6.2 Hz, 1H), 4.21 (d, J = 5.7 Hz, |
| | | | 2H), 3.73 (d, J = 1.6 Hz, 3H), 3.60 (d, J = 13.0 |
| | | | Hz, 1H), 3.40 (t, J = 8.2 Hz, 1H), 3.05-2.90 (m, |
| | | | 2H), 2.18 (q, J = 8.9, 8.2 Hz, 2H), 1.74 (dd, J = |
| | | | 16.7, 9.0 Hz, 2H), 1.56 (q, J = 9.7, 8.6 Hz, 1H). |
| 203 | 3.1 | (S)-2-phenyl- pyrrolidine | 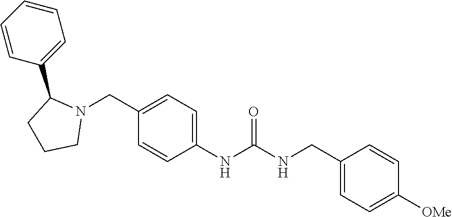 [SMILES: COc1ccc(CNC(=O)Nc2ccc(CN3CCC[C@H]3c3ccccc3)cc2)cc1] |
| | | | Compound 203: N-{4-[((2S)-2- |
| | | | phenylpyrrolidinyl)methyl]phenyl}{[(4- |
| | | | methoxyphenyl)methyl]amino}carboxamide. |
| | | | LCMS-ESI (POS.) m/z: 416.20 (M + H)+. ¹H |
| | | | NMR (400 MHz, DMSO-d₆) δ 8.59 (s, 1H), 7.40 |
| | | | (dd, J = 13.2, 7.2 Hz, 4H), 7.39-7.21 (m, 3H), |
| | | | 7.19 (d, J = 8.6 Hz, 2H), 7.09 (d, J = 8.4 Hz, |
| | | | 2H), 6.86 (d, J = 8.6 Hz, 2H), 6.60 (t, J = 6.0 Hz, |
| | | | 1H), 4.38 (ddd, J = 32.8, 16.6, 10.1 Hz, 1H), |
| | | | 4.28 (s, 2H), 4.04-3.89 (m, 1H), 3.83 (d, J = |
| | | | 9.0 Hz, 1H), 3.81-3.64 (m, 1H), 3.68 (s, 3H), |
| | | | 3.46 (d, J = 12.9 Hz, 1H), 3.19-3.04 (m, 1H), |
| | | | 2.24 (dt, J = 12.9, 6.3 Hz, 1H), 1.86 (tt, J = 18.6, |
| | | | 8.6 Hz, 3H). |
| 208 | 3.1 | 2-phenyl- pyrrolidine | 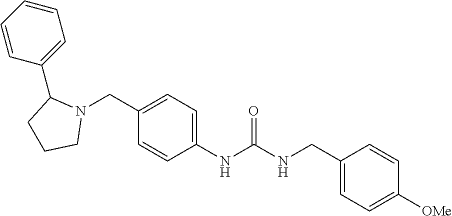 [SMILES: COc1ccc(CNC(=O)Nc2ccc(CN3CCCC3c3ccccc3)cc2)cc1] |
| | | | Compound 208: {[(4- |
| | | | methoxyphenyl)methyl]amino}-N-{4-[(2- |
| | | | phenylpyrrolidinyl)methyl]phenyl}carboxamide. |
| | | | LCMS-ESI (POS.) m/z: 416.20 (M + H)+. ¹H |
| | | | NMR (400 MHz, DMSO-d₆) δ 8.59 (s, 1H), 7.40 |
| | | | (dd, J = 13.2, 7.2 Hz, 4H), 7.39-7.21 (m, 3H), |
| | | | 7.19 (d, J = 8.6 Hz, 2H), 7.09 (d, J = 8.4 Hz, |
| | | | 2H), 6.86 (d, J = 8.6 Hz, 2H), 6.60 (t, J = 6.0 Hz, |
| | | | 1H), 4.38 (ddd,J = 32.8, 16.6, 10.1 Hz, 1H), |
| | | | 4.28 (s, 2H), 4.04-3.89 (m, 1H), 3.83 (d, J = |
| | | | 9.0 Hz, 1H), 3.81-3.64 (m, 1H), 3.68 (s, 3H), |
| | | | 3.46 (d, J = 12.9 Hz, 1H), 3.19-3.04 (m, 1H), |
| | | | 2.24 (dt, J = 12.9, 6.3 Hz, 1H), 1.86 (tt, J = 18.6, |
| | | | 8.6 Hz, 3H). |
| 151 | 3.3 | 2- (pyrrolidin-2- yl)pyridine | 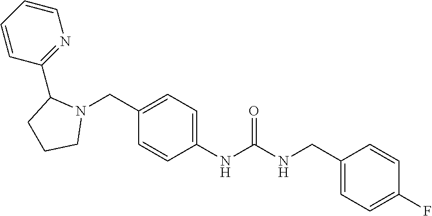 [SMILES: O=C(NCc1ccc(F)cc1)Nc1ccc(CN2CCCC2c2ccccn2)cc1] |
| | | | Compound 151: N-1(4- |
| | | | fluorophenyl)methyl]({4-[(2-(2- |
| | | | pyridyl)pyrrolidinyl)methyl]phenyl}amino) |
| | | | carboxamide. LCMS-ESI (POS.) m/z: 405.20 |
| | | | (M + H)+. ¹H NMR (400 MHz, DMSO-d₆) δ 8.55 |
| | | | (s, 1H), 8.49 (d, J = 4.8 Hz, 1H), 7.81 (td, J = |
| | | | 1.1, 1.8 Hz, 1H), 7.63 (d, J = 7.9 Hz, 1H), 7.38- |
| | | | 7.29 (m, 4H), 7.26 (dd, J = 7.4, 4.9 Hz, 1H), |
| | | | 7.21-7.08 (m, 4H), 6.62 (t, J = 6.0 Hz, 1H), |
| | | | 4.27 (d, J = 5.9 Hz, 2H), 3.67-3.53 (m, 2H), |
| | | | 3.10 (d, J = 13.0 Hz, 1H), 2.96 (ddd, J = 9.1, 6.8, |
| | | | 3.5 Hz, 1H), 2.30-2.14 (m, 2H), 1.84-1.71 |
| | | | (m, 2H), 1.66 (dtd, J = 17.8, 9.7, 5.3 Hz, 1H). |
| 355 | 3.1 | 3- (pyrrolidin-2- yl)pyridine | 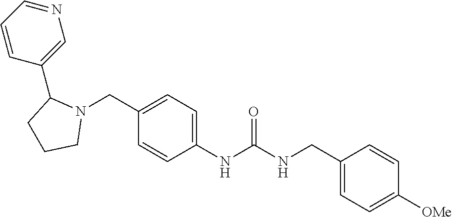 [SMILES: COc1ccc(CNC(=O)Nc2ccc(CN3CCCC3c3cccnc3)cc2)cc1] |
| | | | 1-(4-methoxybenzyl)-3-(4-((2-(pyridin-3- |
| | | | yl)pyrrolidin-1-yl)methyl)phenyl)urea. |
| | | | LCMS-ESI (POS.) m/z: 417.2 (M + H)+. ¹H |
| | | | NMR (400 MHz, DMSO-d₆) δ 8.61 (d, J = 2.2 |
| | | | Hz, 1H), 8.52-8.35 (m, 2H), 7.85 (d, J = 7.8 |
| | | | Hz, 1H), 7.43-7.24 (m, 3H), 7.23 (d, J = 8.5 |
| | | | Hz, 2H), 7.09 (d, J = 8.3 Hz, 2H), 6.90 (d, J = |
| | | | 8.8 Hz, 2H), 6.48 (t, J = 5.9 Hz, 1H), 4.22 (d, J = |
| | | | 5.9 Hz, 2H), 3.73 (s, 3H), 3.58 (d, J = 13.0 Hz, |
| | | | 1H), 3.48-3.41 (m, 1H), 3.12-2.86 (m, 2H), |
| | | | 2.21 (pd, J = 8.8, 4.7 Hz, 2H), 1.92-1.70 (m, |
| | | | 2H), 1.61 (dtd, J = 18.7, 10.4, 9.7, 6.2 Hz, 1H). |
| 356 | 3.1 | 4- (pyrrolidin-2- yl)pyridine | 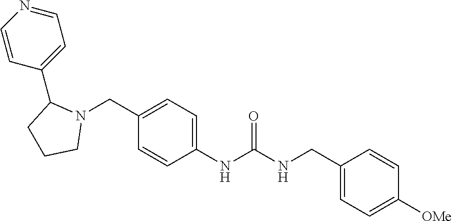 [SMILES: COc1ccc(CNC(=O)Nc2ccc(CN3CCCC3c3ccncc3)cc2)cc1] |
| | | | 1-(4-methoxybenzyl)-3-(4-((2-(pyridin-4- |
| | | | yl)pyrrolidin-1-yl)methyl)phenyl)urea. |
| | | | LCMS-ESI (POS.) m/z: 417.2 (M + H)+. ¹H |
| | | | NMR (400 MHz, DMSO-d₆) δ 8.53 (d, J = 5.6 |
| | | | Hz, 2H), 8.47 (s, 1H), 7.45 (d, J = 5.9 Hz, 2H), |
| | | | 7.33 (d, J = 8.4 Hz, 2H), 7.23 (d, J = 8.7 Hz, |
| | | | 2H), 7.12 (d, J = 8.3 Hz, 2H), 6.90 (d, J = 8.6 |
| | | | Hz, 2H), 6.49 (t, J = 5.9 Hz, 1H), 4.22 (d, J = 5.8 |
| | | | Hz, 2H), 3.74 (s, 3H), 3.60 (d, J = 12.9 Hz, 1H), |
| | | | 3.43 (t, J = 8.2 Hz, 1H), 3.05 (d, J = 12.9 Hz, |
| | | | 1H), 2.96 (ddd, J =9.8, 7.1, 3.4 Hz, 1H), 2.21 |
| | | | (dq, J = 12.9, 8.6 Hz, 2H), 1.76 (tdd, J = 9.2, 6.2, |
| | | | 3.0 Hz, 2H), 1.54 (dtd, J = 12.4, 9.5, 6.5 Hz, |
| | | | 1H). |
| 357 | 3.1 | 2-phenyl- azetidine | 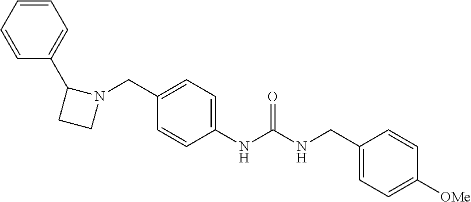 [SMILES: COc1ccc(CNC(=O)Nc2ccc(CN3CCC3c3ccccc3)cc2)cc1] |
| | | | 1-(4-methoxybenzyl)-3-(4-((2-phenylazetidin- |
| | | | 1-yl)methyl)phenyl)urea. LCMS-ESI (POS.) |
| | | | m/z: 402.2 (M + H)+. ¹H NMR (400 MHz, |
| | | | DMSO-d₆) δ 8.47 (s, 1H), 7.43 (d, J = 7.4 Hz, |
| | | | 2H), 7.37-7.27 (m, 4H), 7.24 (t, J = 7.5 Hz, |
| | | | 3H), 7.13 (d, J = 8.3 Hz, 2H), 6.90 (d, J = 8.5 |
| | | | Hz, 2H), 6.51 (t, J = 5.8 Hz, 1H), 4.22 (d, J = 5.8 |
| | | | Hz, 2H), 4.12 (t, J = 8.1 Hz, 1H), 3.74 (s, 3H), |
| | | | 3.65 (d, J = 12.8 Hz, 1H), 3.33 (d, J = 12.9 Hz, |
| | | | 1H), 3.17 (t, J = 6.6 Hz, 1H), 2.84 (dt, J = 9.6, |
| | | | 7.1 Hz, 1H), 2.29 (dtd, J = 9.6, 7.5, 1.9 Hz, 1H), |
| | | | 1.94 (p, J = 9.2 Hz, 1H). |
| 358 | 3.1 | 2-(pyridin-2- yl)pyrrolidin- 3-ol | 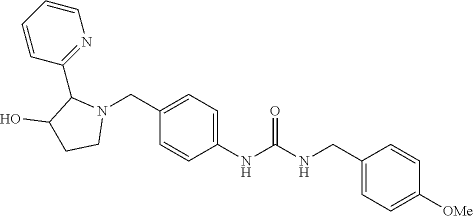 [SMILES: COc1ccc(CNC(=O)Nc2ccc(CN3CCC(O)C3c3ccccn3)cc2)cc1] |
| | | | 1-(4-((3-hydroxy-2-(pyridin-2-yl)pyrrolidin-1- |
| | | | yl)methyl)phenyl)-3-(4-methoxybenzyl)urea. |
| | | | LCMS-ESI (POS.) m/z: 433.2 (M + H)+. |
| 359 | 3.1 | 2-(pyridin- 3-yl) pyrrolidin- 3-ol | 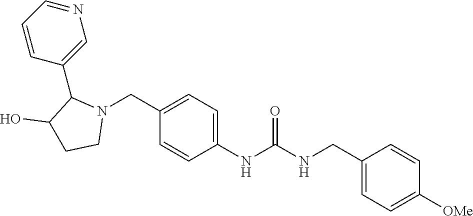 [SMILES: COc1ccc(CNC(=O)Nc2ccc(CN3CCC(O)C3c3cccnc3)cc2)cc1] |
| | | | 1-(4-((3-hydroxy-2-(pyridin-3-yl)pyrrolidin-1- |
| | | | yl)methyl)phenyl)-3-(4-methoxybenzyl)urea. |
| | | | LCMS-ESI (POS.) m/z: 433.2 (M + H)+. |
| 434 | 3.2 | thiomor- pholine 1,1- dioxide | 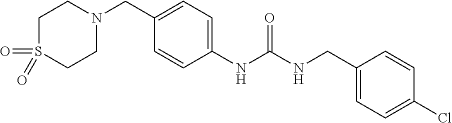 [SMILES: O=C(NCc1ccc(Cl)cc1)Nc1ccc(CN2CCS(=O)(=O)CC2)cc1] |
| | | | 1-(4-chlorobenzyl)-3-(4-((1,1- |
| | | | dioxidothiomorpholino)methyl)phenyl)urea. |
| | | | LCMS-ESI (POS.) m/z: 816.1 (2M + H)+. |
| | | | ¹H NMR (400 MHz, Methanol-d₄) δ 7.34 (d, J = |
| | | | 7.0 Hz, 6H), 7.26 (d, J = 8.1 Hz, 2H), 4.38 (s, |
| | | | 2H), 3.63 (s, 2H), 3.11 (t, J = 5.1 Hz, 4H), 2.97 |
| | | | (dd, J = 7.1, 3.6 Hz, 4H). |
| 436 | 3.2 | (S)-3-(methyl- sulfonyl) pyrrolidine | 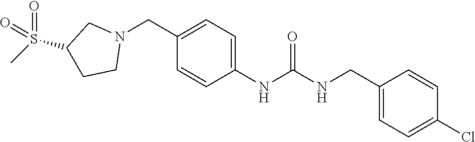 |
| | | | (S)-1-(4-chlorobenzyl)-3-(4-((3- |
| | | | (methylsulfonyl)pyrrolidin-1- |
| | | | yl)methyl)phenyl)urea. |
| | | | LCMS-ESI (POS.) m/z: 422.1 (M + H)+. |
| | | | ¹H NMR (400 MHz, Methanol-d₄) δ 7.40-7.17 |
| | | | (m, 8H), 4.38 (s, 2H), 3.73 (q, J = 7.6 Hz, 1H), |
| | | | 3.67-3.52 (m, 2H), 2.94-2.87 (m, 5H), 2.76- |
| | | | 2.60 (m, 2H), 2.28-2.17 (m, 2H). |
| 437 | 3.2 | (R)-3-(methyl- sulfonyl) pyrrolidine | 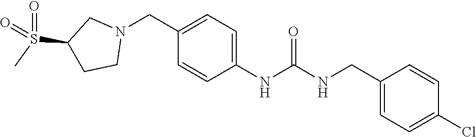 [SMILES: CS(=O)(=O)[C@@H]1CCN(Cc2ccc(NC(=O)NCc3ccc(Cl)cc3)cc2)C1] |
| | | | (R)-1-(4-chlorobenzyl)-3-(4-((3- |
| | | | (methylsulfonyl)pyrrolidin-1- |
| | | | yl)methyl)phenyl)urea. |
| | | | LCMS-ESI (POS.) m/z: 422.1 (M + H)+. |
| | | | ¹H NMR (400 MHz, Methanol-d₄) δ 7.35 (d, J = |
| | | | 10.9 Hz, 6H), 7.26 (d, J = 8.0 Hz, 2H), 4.39 (s, |
| | | | 2H), 3.76 (p, J = 7.4 Hz, 1H), 3.70-3.54 (m, |
| | | | 2H), 3.10 (dd, J = 49.9, 7.4 Hz, 1H), 2.92 (d, J = |
| | | | 8.2 Hz, 4H), 2.78-2.63 (m, 2H), 2.31-2.19 |
| | | | (m, 2H). |
| 439 | 3.1 | (S)-3-(methyl sulfonyl) pyrrolidine | 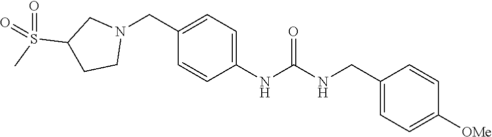 [SMILES: COc1ccc(CNC(=O)Nc2ccc(CN3CCC(S(C)(=O)=O)C3)cc2)cc1] |
| | | | (S)-1-(4-methoxybenzyl)-3-(4-((3- |
| | | | (methylsulfonyl)pyrrolidin-1- |
| | | | yl)methyl)phenyl)urea. |
| | | | LCMS-ESI (POS.) m/z: 418.1 (M + H)+. |
| | | | ¹H NMR (400 MHz, Methanol-d₄) δ 7.35 (d, J = |
| | | | 7.8 Hz, 2H), 7.26 (t, J = 6.1 Hz, 4H), 6.90 (d, J = |
| | | | 7.8 Hz, 2H), 3.79 (s, 3H), 3.78-3.70 (m, 1H), |
| | | | 3.68-3.53 (m, 2H), 2.92 (d, J = 8.4 Hz, 5H), |
| | | | 2.69 (tt, J = 16.1, 8.5 Hz, 2H), 2.25 (q, J = 7.1 |
| | | | Hz, 2H). |
| 438 | 3.1 | (R)-3-(methyl- sulfonyl) pyrrolidine | 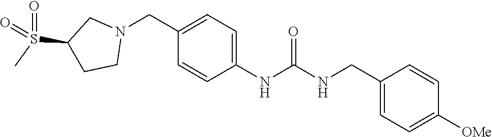 [SMILES: COc1ccc(CNC(=O)Nc2ccc(CN3CC[C@@H](S(C)(=O)=O)C3)cc2)cc1] |
| | | | (R)-1-(4-methoxybenzyl)-3-(4-((3- |
| | | | (methylsulfonyl)pyrrolidin-1- |
| | | | yl)methyl)phenyl)urea. |
| | | | LCMS-ESI (POS.) m/z: 418.1 (M + H)+ ¹H |
| | | | NMR (400 MHz, Methanol-d₄) δ 7.35 (d, J = 8.1 |
| | | | Hz, 2H), 7.26 (t, J = 6.1 Hz, 4H), 6.90 (d, J = 7.8 |
| | | | Hz, 2H), 4.33 (s, 2H), 3.79 (s, 3H), 3.78-3.70 |
| | | | (m, 1H), 3.69-3.52 (m, 2H), 2.92 (d, J = 8.6 |
| | | | Hz, 5H), 2.80-2.60 (m, 2H), 2.25 (q, J = 7.1 |
| | | | Hz, 2H). |
| | 3.2 | 3-aminotetra- hydrothiophene 1,1-dioxide | 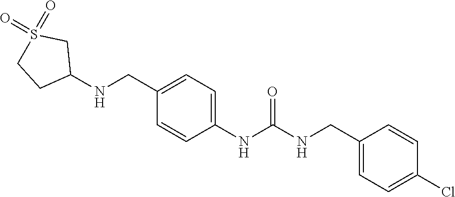 [SMILES: O=C(NCc1ccc(Cl)cc1)Nc1ccc(CNC2CCS(=O)(=O)C2)cc1] |
| | | | 1-(4-chlorobenzyl)-3-(4-(((1,1- |
| | | | dioxidotetrahydrothiophen-3- |
| | | | yl)amino)methyl)phenyl)urea. LCMS-ESI |
| | | | (POS.) m/z: 408 (M + H)+. |
| | 3.2 | piperazin-2- one | 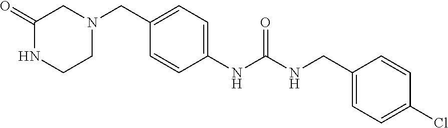 [SMILES: O=C1CN(Cc2ccc(NC(=O)NCc3ccc(Cl)cc3)cc2)CCN1] |
| | | | 1-(4-chlorobenzyl)-3-(4-((3-oxopiperazin-1- |
| | | | yl)methyl)phenyl)urea. LCMS-ESI (POS.) |
| | | | m/z: 373 (M + H)⁺. ¹H NMR (400 MHz, DMSO- |
| | | | d₆) δ 8.58 (s, 1H), 7.71 (s, 1H), 7.45-7.29 (m, |
| | | | 6H), 7.17 (d, J = 8.3 Hz, 2H), 6.64 (t, J = 6.1 Hz, |
| | | | 1H), 4.29 (d, J = 6.0 Hz, 2H), 3.45 (s, 2H), 3.30 |
| | | | (s, 2H), 3.13 (d, J = 6.1 Hz, 2H), 2.87 (s, 2H) |
| 364 | 3.2 | 1-methyl piperazin-2- one | 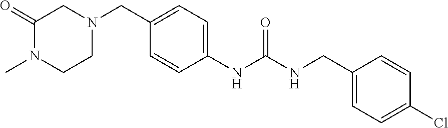 [SMILES: CN1CCN(Cc2ccc(NC(=O)NCc3ccc(Cl)cc3)cc2)CC1=O] |
| | | | 1-(4-chlorobenzyl)-3-(4-((4-methyl-3- |
| | | | oxopiperazin-1-yl)methyl)phenyl)urea. |
| | | | LCMS-ESI (POS.) m/z: 387 (M + H)⁺. ¹H NMR |
| | | | (300 MHz, DMSO-d₆) δ 8.59 (s, 1H), 7.45- |
| | | | 7.29 (m, 6H), 7.17 (d, J = 8.3 Hz, 2H), 6.64 (t, J = |
| | | | 6.0 Hz, 1H), 4.29 (d, J = 6.0 Hz, 2H), 3.44 (s, |
| | | | 2H), 3.25 (d, J = 11.2 Hz, 2H), 2.92 (s, 2H), 2.82 |
| | | | (s, 3H), 2.61 (t, J = 5.6 Hz, 2H). |
| 386 | 3.2 | methyl piperazine-1- carboxylate | 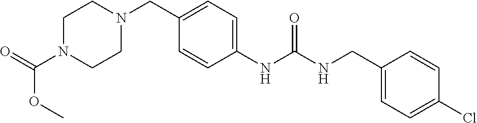 [SMILES: COC(=O)N1CCN(Cc2ccc(NC(=O)NCc3ccc(Cl)cc3)cc2)CC1] |
| | | | Methyl 4-(4-(3-(4- |
| | | | chlorobenzyl)ureido)benzyl)piperazine-1- |
| | | | carboxylate. LCMS-ESI (POS.) m/z: 417 |
| | | | (M + H)⁺. ¹H NMR (300 MHz, DMSO-d₆) δ 8.55 |
| | | | (s, 1H), 7.42-7.26 (m, 6H), 7.13 (d, J = 8.1 Hz, |
| | | | 2H), 6.63 (d, J = 6.2 Hz, 1H), 4.27 (d, J = 5.8 |
| | | | Hz, 2H), 3.57 (s, 3H), 3.35 (s, 2H), 3.30 (s, 4H), |
| | | | 2.28 (t, J = 5.2 Hz, 4H). |
| 387 | 3.1 | methyl piperazine-1- carboxylate | 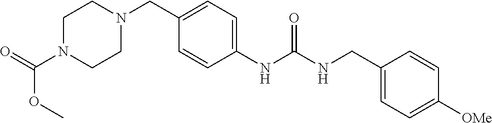 [SMILES: COC(=O)N1CCN(Cc2ccc(NC(=O)NCc3ccc(OC)cc3)cc2)CC1] |
| | | | Methyl 4-(4-(3-(4- |
| | | | methoxybenzyl)ureido)benzyl)piperazine-1- |
| | | | carboxylate. LCMS-ESI (POS.) m/z: 413 |
| | | | (M + H)⁺. ¹H NMR (300 MHz, DMSO-d₆) δ 8.47 |
| | | | (s, 1 H), 7.34 (d, J = 8.4 Hz, 2 H), 7.22 (d, J = |
| | | | 8.6 Hz, 2 H), 7.13 (d, J = 8.2 Hz, 2 H), 6.89 (d, J = |
| | | | 8.6 Hz, 2 H), 6.49 (t, J = 5.9 Hz, 1H), 4.21 (d, |
| | | | J = 5.8 Hz, 2 H), 3.73 (s, 3 H), 3.58 (s, 3 H), |
| | | | 3.38 (s, 2 H), 3.36-3.30 (m, 4 H), 2.36-2.22 (m, |
| | | | 4H). |
| 547 | Intermediate 3.2 | 8-oxa-3- azabicyclo [3.2.1]octane | 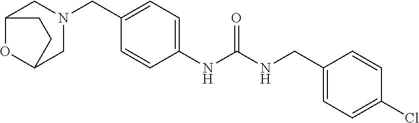 [SMILES: O=C(NCc1ccc(Cl)cc1)Nc1ccc(CN2CC3CCC(C2)O3)cc1] |
| | | | 1-(4-((8-oxa-3-azabicyclo[3.2.1]octan-3- |
| | | | yl)methyl)phenyl)-3-(4-chlorobenzyl)urea. |
| | | | LCMS-ESI (POS.) m/z: 386.10 (M + H)⁺. ¹H |
| | | | NMR (400 MHz, DMSO-d₆) δ 8.55 (s, 1 H), |
| | | | 7.39 (d, J = 8.5 Hz, 2 H), 7.36-7.29 (m, 4 H), |
| | | | 7.13 (d, J = 8.2 Hz, 2H), 6.63 (t, J = 6.0 Hz, 1 |
| | | | H), 4.28 (d, J = 6.0 Hz, 2 H), 4.19 (dt, J = 4.8, |
| | | | 2.3 Hz, 2 H), 2.51 (p, J = 1.8 Hz, 2 H), 2.47 (d, J = |
| | | | 10.5 Hz, 2H), 2.15 (dd, J = 11.1, 2.0 Hz, 2 H), |
| | | | 1.84 (t, J = 6.0 Hz, 2 H), 1.70 (dd, J = 7.8, 4.2 |
| | | | Hz, 2 H). |
| 454 | Intermediate 3.2 | 2-oxa-5- azaspiro[3.4] octane | 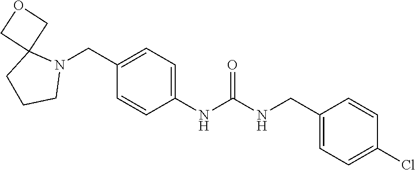 [SMILES: O=C(NCc1ccc(Cl)cc1)Nc1ccc(CN2CCCC23COC3)cc1] |
| | | | 1-(4-((2-oxa-5-azaspiro[3.4]octan-5- |
| | | | yl)methyl)phenyl)-3-(4-chlorobenzyl)urea. |
| | | | LCMS-ESI (POS.) m/z: 386.10 (M + H)⁺. ¹H |
| | | | NMR (400 MHz, DMSO-d₆) δ 8.56 (s, 1 H), |
| | | | 7.39 (d, J = 8.5 Hz, 2 H), 7.37-7.30 (m, 4 H), |
| | | | 7.18 (d, J = 8.3 Hz, 2H), 6.63 (t, J = 6.0 Hz, 1 |
| | | | H), 4.79 (d, J = 6.5 Hz, 2 H), 4.41 (d, J = 6.5 Hz, |
| | | | 2 H), 4.28 (d, J = 5.9 Hz, 2 H), 3.86 (s, 2 H), |
| | | | 2.47 (t, J = 7.0 Hz, 2 H), 2.17-2.06 (m, 2 H), |
| | | | 1.61 (p, J =7.2 Hz, 2 H). |
| 458 | Intermediate 3.2 | 2-oxa-5- azaspiro[3.5] nonane | 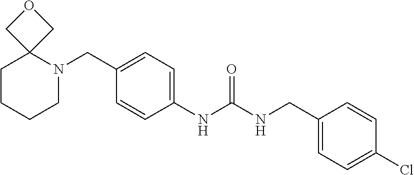 [SMILES: O=C(NCc1ccc(Cl)cc1)Nc1ccc(CN2CCCCC23COC3)cc1] |
| | | | 1-(4-((2-oxa-5-azaspiro [3.5] nonan-5- |
| | | | yl)methyl)phenyl)-3-(4-chlorobenzyl)urea. |
| | | | LCMS-ESI (POS.) m/z: 400.1 (M + H)⁺. ¹H NMR |
| | | | (400 MHz, Methanol-d₄) δ 7.44 (d, J = 8.3 Hz, 2 |
| | | | H), 7.37 (d, J = 8.2 Hz, 2 H), 7.35-7.32 (m, 4 |
| | | | H), 4.86 (d, J = 7.1 Hz, 2 H), 4.47 (d, J = 7.2 Hz, |
| | | | 2 H), 4.39 (s, 2 H), 4.10-4.00 (m, 2 H), 2.80- |
| | | | 2.68 (m, 2 H), 2.19-2.09 (m, 2 H), 1.74-1.58 (m, |
| | | | 4H). |
| 459 | Intermediate 3.2 | 7-oxa-4- azaspiro[2.5] octane | 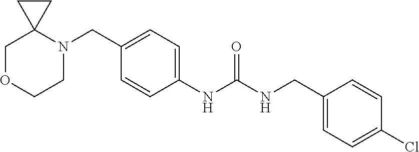 [SMILES: O=C(NCc1ccc(Cl)cc1)Nc1ccc(CN2CCOCC23CC3)cc1] |
| | | | 1-(4-((7-oxa-4-azaspiro[2.5]octan-4- |
| | | | yl)methyl)phenyl)-3-(4-chlorobenzyl)urea. |
| | | | LCMS-ESI (POS.) m/z: 386.10 (M + H)⁺. ¹H |
| | | | NMR (400 MHz, DMSO-d₆) δ 8.54 (s, 1 H), |
| | | | 7.39 (d, J = 8.4 Hz, 2 H), 7.35-7.28 (m, 4 H), |
| | | | 7.11 (d, J = 8.3 Hz, 2H), 6.61 (t, J = 6.1 Hz, 1 |
| | | | H), 4.27 (d, J = 5.9 Hz, 2 H), 3.68 (s, 2 H), 3.61 |
| | | | (t, J = 4.7 Hz, 2 H), 3.43 (s, 2 H), 2.62 (t, J = 4.7 |
| | | | Hz, 2 H), 0.69-0.63 (m, 2 H), 0.49-0.43 (m, 2 |
| | | | H). |
| 464 | Intermediate 3.2 | 4- (methyl- sulfonyl) piperidine | 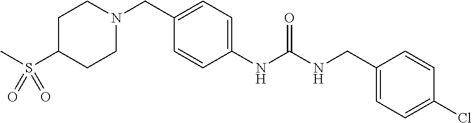 [SMILES: CS(=O)(=O)C1CCN(Cc2ccc(NC(=O)NCc3ccc(Cl)cc3)cc2)CC1] |
| | | | 1-(4-chlorobenzyl)-3-(4-((4- |
| | | | (methylsulfonyl)piperidin-1- |
| | | | yl)methyl)phenyl)urea. LCMS-ESI (POS.) |
| | | | m/z: 436.0 (M + H)⁺. ¹H NMR (400 MHz, |
| | | | DMSO-d₆) δ 8.56 (s, 1 H), 7.39 (d, J = 8.4 Hz, 2 |
| | | | H), 7.37-7.29 (m, 4 H), 7.14 (d, J = 8.3 Hz, 2 |
| | | | H), 6.62 (t, J = 6.1 Hz, 1 H), 4.28 (d, J = 5.9 Hz, |
| | | | 2 H), 3.38 (s, 2 H), 3.02 (t, J = 12.4 Hz, 1 H), |
| | | | 2.95-2.86 (m, 2 H), 2.91 (s, 3 H), 2.01-1.86 (m, |
| | | | 4H), 1.58 (qd, J = 12.5, 3.7 Hz, 2 H). |
| 465 | Intermediate 3.2 | 6-thia-1- azaspiro[3.3] heptane 6,6-dioxide | 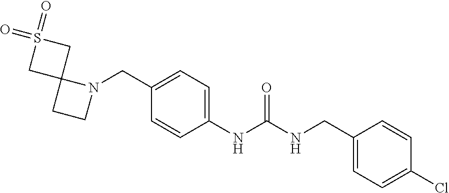 [SMILES: O=C(NCc1ccc(Cl)cc1)Nc1ccc(CN2CCC23CS(=O)(=O)C3)cc1] |
| | | | 1-(4-chlorobenzyl)-3-(4-((6,6-dioxido-6-thia-1- |
| | | | azaspiro[3.3]heptan-1-yl)methyl)phenyl)urea. |
| | | | LCMS-ESI (POS.) m/z: 419.80 (M + H)⁺. ¹H |
| | | | NMR (400 MHz, DMSO-d₆) δ 8.56 (s, 1 H), |
| | | | 7.39 (d, J = 8.5 Hz, 2 H), 7.37-7.29 (m, 4 H), |
| | | | 7.17 (d, J = 8.3 Hz, 2 H), 6.63 (t, J = 6.0 Hz, 1 |
| | | | H), 4.46 (d, J = 15.0 Hz, 2 H), 4.33-4.19 (m, 4 |
| | | | H), 3.57 (s, 2 H), 2.98 (t, J = 6.7 Hz, 2 H), 2.37 |
| | | | (t, J = 6.7 Hz, 2H). |
| 466 | Intermediate 3.2 | 2,8-dioxa- 5-azaspiro [3.5]nonane | 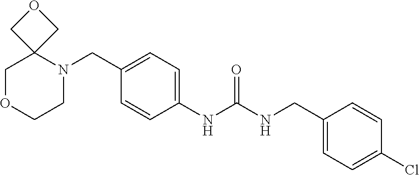 [SMILES: O=C(NCc1ccc(Cl)cc1)Nc1ccc(CN2CCOCC23COC3)cc1] |
| | | | 1-(4-((2,8-dioxa-5-azaspiro[3.5]nonan-5- |
| | | | yl)methyl)phenyl)-3-(4-chlorobenzyl)urea. |
| | | | LCMS-ESI (POS.) m/z: 401.8 (M + H)⁺. ¹H NMR |
| | | | (400 MHz, DMSO-d₆) δ 8.56 (s, 1 H), 7.39 (d, J = |
| | | | 8.5 Hz, 2 H), 7.35 (d, J = 8.5 Hz, 2 H), 7.32 (d, |
| | | | J = 8.5 Hz, 2 H), 7.20 (d, J = 8.5 Hz, 2 H), 6.62 |
| | | | (t, J = 6.1 Hz, 1 H), 4.77 (d, J = 6.9 Hz, 2 H), |
| | | | 4.28 (d, J = 6.0 Hz, 2 H), 4.24 (d, J = 6.9 Hz, 2 |
| | | | H), 3.83 (s, 2 H), 3.76 (s, 2 H), 3.55-3.47 (m, 2 |
| | | | H), 2.33-2.26 (m, 2 H). |
| 548 | Intermediate 3.2 | 3- methyl- azetidine-3- carbonitrile | 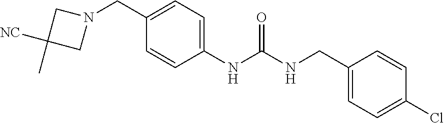 [SMILES: CC1(C#N)CN(Cc2ccc(NC(=O)NCc3ccc(Cl)cc3)cc2)C1] |
| | | | 1-(4-chlorobenzyl)-3-(4-((3-cyano-3- |
| | | | methylazetidin-1-yl)methyl)phenyl)urea. |
| | | | LCMS-ESI (POS.) m/z: 369.10 (M + H)⁺. ¹H |
| | | | NMR (400 MHz, DMSO-d₆) δ 8.58 (s, 1 H), |
| | | | 7.39 (d, J = 8.4 Hz, 2 H), 7.37-7.29 (m, 4 H), |
| | | | 7.11 (d, J = 8.2 Hz, 2H), 6.63 (t, J = 6.1 Hz, |
| | | | 1H), 4.28 (d, J = 5.9 Hz, 2 H), 3.49 (s, 2 H), 3.43 |
| | | | (d, J = 6.5 Hz, 2 H), 3.10 (d, J = 7.0 Hz, 2 H), |
| | | | 1.52 (s, 3H). |
| 471 | Intermediate 3.2 | 2-oxa-6- azaspiro[3.5] nonane | 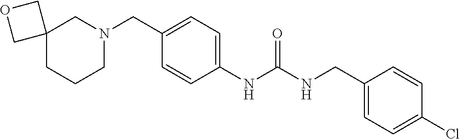 [SMILES: O=C(NCc1ccc(Cl)cc1)Nc1ccc(CN2CCCC3(COC3)C2)cc1] |
| | | | 1-(4-((2-oxa-6-azaspiro[3.5]nonan-6- |
| | | | yl)methyl)phenyl)-3-(4-chlorobenzyl)urea. |
| | | | LCMS-ESI (POS.) m/z: 400.15 (M + H)⁺. ¹H |
| | | | NMR (400 MHz, DMSO-d₆) δ 8.57 (s, 1 H), |
| | | | 7.39 (d, J = 8.4 Hz, 2 H), 7.36 (d, J = 8.2 Hz, 2 |
| | | | H), 7.32 (d, J = 8.4 Hz, 2 H), 7.14 (d, J = 8.2 Hz, |
| | | | 2 H), 6.63 (t, J = 6.1 Hz, 1 H), 4.28 (d, J = 6.0 |
| | | | Hz, 2H), 4.22 (d, J = 5.7 Hz, 2 H), 4.15 (d, J = |
| | | | 5.7 Hz, 2 H), 3.39 (s, 2 H), 2.42 (s, 2 H), 2.31- |
| | | | 2.16 (m, 2 H), 1.67-1.55 (m, 2 H), 1.48-1.37 (m, |
| | | | 2H). |
| 479 | Intermediate 3.2 | 1- (methyl- sulfonyl) piperazine | 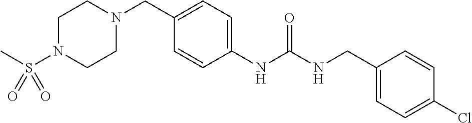 [SMILES: CS(=O)(=O)N1CCN(Cc2ccc(NC(=O)NCc3ccc(Cl)cc3)cc2)CC1] |
| | | | 1-(4-chlorobenzyl)-3-(4-((4- |
| | | | (methylsulfonyl)piperazin-1- |
| | | | yl)methyl)phenyl)urea. LCMS-ESI (POS.) |
| | | | m/z: 437.0 (M + H)⁺. ¹H NMR (400 MHz, |
| | | | DMSO-d₆) δ 8.58 (s, 1 H), 7.39 (d, J = 8.5 Hz, 2 |
| | | | H), 7.35 (d, J = 8.2 Hz, 2 H), 7.32 (d, J = 8.5 Hz, |
| | | | 2 H), 7.15 (d, J = 8.2 Hz, 2 H), 6.63 (t, J = 6.0 |
| | | | Hz, 1 H), 4.28 (d, J = 5.9 Hz, 2 H), 3.42 (s, 2 H), |
| | | | 3.09 (t, J = 4.9 Hz, 4 H), 2.86 (s, 3 H), 2.42 (t, J = |
| | | | 4.9 Hz, 4 H). |
| 536 | Intermediate 3.1 | 1- (methyl- sulfonyl) piperazine | 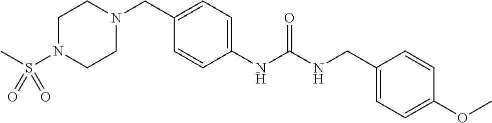 |
| | | | 1-(4-methoxybenzyl)-3-(4-((4- |
| | | | (methylsulfonyl)piperazin-1- |
| | | | yl)methyl)phenyl)urea. LCMS-ESI (POS.) |
| | | | m/z: 433.2 (M + H)⁺. ¹H NMR (400 MHz, |
| | | | DMSO-d₆) δ 8.48 (s, 1 H), 7.35 (d, J = 8.5 Hz, 2 |
| | | | H), 7.22 (d, J = 8.6 Hz, 2 H), 7.14 (d, J = 8.4 Hz, |
| | | | 2 H), 6.89 (d, J = 8.7 Hz, 2 H), 6.49 (t, J = 5.9 |
| | | | Hz, 1 H), 4.21 (d, J = 5.8 Hz, 2 H), 3.73 (s, 3 H), |
| | | | 3.42 (s, 2 H), 3.09 (t, J = 4.9 Hz, 4 H), 2.86 (s, 3 |
| | | | H), 2.42 (t, J = 4.9Hz, 4H). |
| 480 | Intermediate 3.2 | (2S,6R)- 2,6- dimethyl- morpholine | 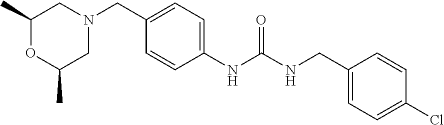 [SMILES: C[C@@H]1CN(Cc2ccc(NC(=O)NCc3ccc(Cl)cc3)cc2)C[C@H](C)O1] |
| | | | 1-(4-chlorobenzyl)-3-(4-(((2S,6R)-2,6- |
| | | | dimethylmorpholino)methyl)phenyl)urea. |
| | | | LCMS-ESI (POS.) m/z: 388.10 (M + H)⁺. ¹H |
| | | | NMR (400 MHz, DMSO-d₆) δ 8.56 (s, 1 H), |
| | | | 7.39 (d, J = 8.5 Hz, 2 H), 7.37-7.29 (m, 4 H), |
| | | | 7.13 (d, J = 8.3 Hz, 2H), 6.63 (t, J = 6.0 Hz, 1 |
| | | | H), 4.28 (d, J = 6.0 Hz, 2 H), 3.53 (ddt, J = 12.8, |
| | | | 6.6, 3.2 Hz, 2 H), 3.34 (s, 2 H), 2.64 (d, J = 10.9 |
| | | | Hz, 2H), 1.59 (t, J = 10.6 Hz, 2 H), 1.01 (d, J = |
| | | | 6.2 Hz, 6 H). |
| 481 | Intermediate 3.2 | (2S,6S)- 2,6- dimethyl- morpholine | 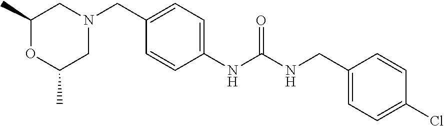 [SMILES: C[C@H]1CN(Cc2ccc(NC(=O)NCc3ccc(Cl)cc3)cc2)C[C@H](C)O1] |
| | | | 1-(4-chlorobenzyl)-3-(4-(((2S,6S)-2,6- |
| | | | dimethylmorpholino)methyl)phenyl)urea. |
| | | | LCMS-ESI (POS.) m/z: 388.15 (M + H)⁺. ¹H |
| | | | NMR (400 MHz, DMSO-d₆) δ 8.56 (s, 1 H), |
| | | | 7.39 (d, J = 8.4 Hz, 2 H), 7.37-7.29 (m, 4 H), |
| | | | 7.14 (d, J = 8.5 Hz, 2 H), 6.63 (t, J = 6.0 Hz, 1 |
| | | | H), 4.28 (d, J = 5.9 Hz, 2 H), 3.88 (pd, J = 6.3, |
| | | | 3.1 Hz, 2H), 3.35 (d, J = 12.9 Hz, 1 H), 3.27 (d, |
| | | | J = 13.0 Hz, 1 H), 2.35 (dd, J = 11.0, 3.2 Hz, 2 |
| | | | H), 2.03 (dd, J = 11.0, 5.7 Hz, 2 H), 1.11 (d, J = |
| | | | 6.4 Hz, 6 H). |
| 482 | Intermediate 3.1 | 1-methyl-3- phenyl- piperazine | 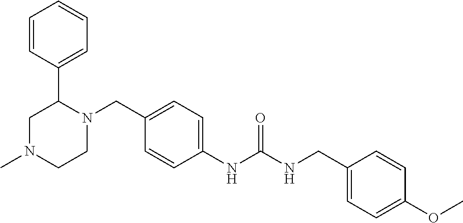 |
| | | | 1-(4-methoxybenzyl)-3-(4-((4-methyl-2- |
| | | | phenylpiperazin-1-yl)methyl)phenyl)urea. |
| | | | LCMS-ESI (POS.) m/z: 445.20 (M + H)⁺. ¹H |
| | | | NMR (400 MHz, DMSO-d₆) δ 8.45 (s, 1 H), |
| | | | 7.48 (d, J = 7.6 Hz, 2 H), 7.37 (t, J = 7.5 Hz, 2 |
| | | | H), 7.33-7.25 (m, 3 H), 7.22 (d, J = 8.6 Hz, 2 |
| | | | H), 7.07 (d, J = 8.2 Hz, 2 H), 6.89 (d, J = 8.7 Hz, |
| | | | 2 H), 6.47 (t, J = 5.8 Hz, 1 H), 4.21 (d, J = 5.8 |
| | | | Hz, 2H), 3.73 (s, 3 H), 3.54 (d, J = 13.1 Hz, 1 |
| | | | H), 3.33-3.28 (m, 1 H), 2.75 (d, J = 13.2 Hz, 1 |
| | | | H), 2.72-2.63 (m, 3H), 2.19-2.07 (m, 1 H), 2.14 |
| | | | (s, 3 H), 2.02 (td, J = 11.5, 2.9 Hz, 1 H), 1.93 (t, |
| | | | J = 10.8 Hz, 1 H). |

### Example 5

### Synthesis of [(4-{(1S)-1-[benzylamino] ethyl}phenyl)amino]-N-[(4-chlorophenyl)methyl]carboxamide (Compound 40)

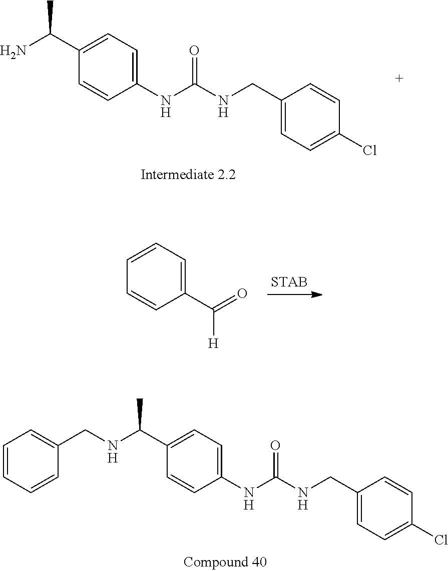 [SMILES: C[C@H](N)c1ccc(NC(=O)NCc2ccc(Cl)cc2)cc1 ; O=Cc1ccccc1 ; C[C@H](NCc1ccccc1)c1ccc(NC(=O)NCc2ccc(Cl)cc2)cc1]

To a solution of benzaldehyde (58 mg, 0.54 mmol, 1.1 equiv) and (S)-1-(4-(1-aminoethyl)phenyl)-3-(4-chlorobenzyl)urea (150 mg, 0.49 mmol, 1.0 equiv) in dichloroethane (2 mL), stirred at room temperature for 1 hour, was added sodium triacetoxyborohydride (209 mg, 0.99 mmol, 2.0 equiv). The resulting solution was stirred at room temperature for 24 hours. A saturated aqueous sodium carbonate solution (3.0 mL) was added and the solution stirred vigorously for 10 mins. The organic layer was separated and the aqueous layer was extracted with ethyl acetate (30 mL). The combined organic layer was washed with brine, dried, filtered, and concentrated under reduced pressure. The crude was purified by reverse phase HPLC with a 10%-100% acetonitrile in water solution that was run over 30 minutes in a Phenomonex Gemini 5u C18 column, providing the desired product (17 mg, 0.04 mmol, 9% yield) as a white solid. LCMS-LCMS-ESI (POS.) m/z: 396.10 (M+H)+. 1H NMR (400 MHz, DMSO-d6) δ 8.58 (s, 1H), 8.18 (s, 1H), 7.62 (t, J=7.6 Hz, 1H), 7.42-7.29 (m, 6H), 7.20 (t, J=8.5 Hz, 3H), 7.09 (d, J=7.6 Hz, 1H), 6.66 (s, 1H), 4.28 (d, J=5.9 Hz, 2H), 3.71 (q, J=6.6 Hz, 1H), 3.59 (s, 2H), 2.43 (s, 3H), 1.29 (d, J=6.5 Hz, 3H).

Compounds in the following table were prepared in a similar manner as Compound 40, using the intermediates and reagents as listed.

| Ex | | | |
|---|---|---|---|
| # | Intermediate | Aldehyde | Structure, Name and Data |
| 77 | 2.2 | 2-(pyridin- 2-yl) acetaldehyde | 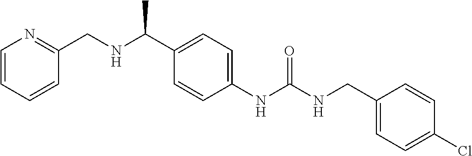 [SMILES: C[C@H](NCc1ccccn1)c1ccc(NC(=O)NCc2ccc(Cl)cc2)cc1] |
| | | | Compound 77: [(4-{(1S)-1-[(2- |
| | | | pyridylmethyl)amino]ethyl}phenyl)amino]-N-[(4- |
| | | | chlorophenyl)methyl]carboxamide. |
| | | | LCMS-ESI (POS.) m/z: 396.10 (M + H)+. |
| | | | ¹H NMR (400 MHz, DMSO-d₆) δ 8.58 (s, |
| | | | 1H), 8.18 (s, 1H), 7.62 (t, J = 7.6 Hz, 1H), |
| | | | 7.42-7.29 (m, 6H), 7.20 (t, J = 8.5 Hz, |
| | | | 3H), 7.09 (d, J = 7.6 Hz, 1H), 6.66 (s, 1H), |
| | | | 4.28 (d, J = 5.9 Hz, 2H), 3.71 (q, J = 6.6 |
| | | | Hz, 1H), 3.59 (s, 2H), 2.43 (s, 3H), 1.29 (d, |
| | | | J = 6.5 Hz, 3H). |
| 70 | 2.2 | 2-(pyridin- 3-yl) acetaldehyde | 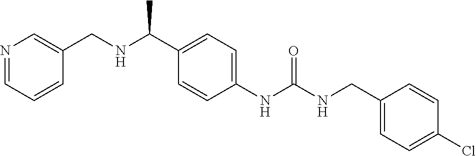 [SMILES: C[C@H](NCc1cccnc1)c1ccc(NC(=O)NCc2ccc(Cl)cc2)cc1] |
| | | | Compound 70: [(4-{(1S)-1-[(3- |
| | | | pyridylmethyl)amino]ethyl}phenyl)amino]-N-[(4- |
| | | | chlorophenyl)methyl]carboxamide. |
| | | | LCMS-ESI (POS.) m/z: 396.10 (M + H)+. |
| | | | ¹H NMR (400 MHz, DMSO-d₆) δ 8.59 (s, |
| | | | 1H), 8.48 (d, J = 4.9 Hz, 1H), 8.18 (d, J = |
| | | | 1.4 Hz, 1H), 7.74 (td, J = 7.7, 1.8 Hz, 1H), |
| | | | 7.45-7.29 (m, 7H), 7.23 (dd, J = 14.9, 7.9 |
| | | | Hz, 3H), 6.67 (s, 1H), 4.28 (d, J = 6.0 Hz, |
| | | | 2H), 3.71 (d, J = 6.4 Hz, 1H), 3.64 (s, 2H), |
| | | | 1.29 (d, J = 6.5 Hz, 3H). |
| 30 | 2.2 | 2-(5- methyl- pyridin-2-yl) acetaldehyde | 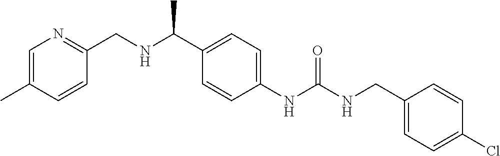 [SMILES: Cc1ccc(CN[C@@H](C)c2ccc(NC(=O)NCc3ccc(Cl)cc3)cc2)nc1] |
| | | | Compound 30: {[4-((1S)-1-{[(5-methyl(2- |
| | | | pyridyl))methyl]amino}ethyl)phenyl]amino}-N-[(4- |
| | | | chlorophenyl)methyl]carboxamide. |
| | | | LCMS-ESI (POS.) m/z: 410.10 (M + H)+. |
| | | | ¹H NMR (400 MHz, DMSO-d₆) δ 8.56 (s, |
| | | | 1H), 8.31 (d, J = 2.1 Hz, 1H), 8.18 (s, 1H), |
| | | | 7.55 (dd, J = 7.8, 2.3 Hz, 1H), 7.42-7.26 |
| | | | (m, 7H), 7.20 (d, J = 8.4 Hz, 2H), 6.65 (t, |
| | | | J = 6.1 Hz, 1H), 4.28 (d, J = 6.0 Hz, 2H), |
| | | | 3.67 (q, J = 6.4 Hz, 1H), 3.58 (s, 2H), 2.27 |
| | | | (s, 3H), 1.27 (d, J = 6.5 Hz, 3H). |
| 28 | 2.2 | 2-(3- methyl- pyridin-2-yl) acetaldehyde | 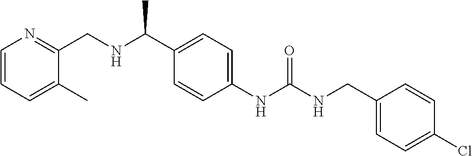 [SMILES: Cc1cccnc1CN[C@@H](C)c1ccc(NC(=O)NCc2ccc(Cl)cc2)cc1] |
| | | | Compound 28: {[4-((1S)-1-{[(3-methyl(2- |
| | | | pyridyl))methyl]amino}ethyl)phenyl]amino}-N-[(4- |
| | | | chlorophenyl)methyl]carboxamide. |
| | | | LCMS-ESI (POS.) m/z: 410.10 (M + H)+. |
| | | | ¹H NMR (400 MHz, DMSO-d₆) δ 8.61 (s, |
| | | | 1H), 8.36 (dd, J = 4.9, 1.6 Hz, 1H), 8.19 (s, |
| | | | 1H), 7.54 (d, J = 7.5 Hz, 1H), 7.43-7.30 |
| | | | (m, 6H), 7.29-7.12 (m, 3H), 6.70 (t, J = |
| | | | 6.0 Hz, 1H), 4.28 (d, J = 6.0 Hz, 2H), 3.77 |
| | | | (q, J = 6.6 Hz, 1H), 3.65 (q, J = 14.3 Hz, |
| | | | 2H), 2.17 (s, 3H), 1.31 (d, J = 6.6 Hz, 3H). |
| 55 | 2.2 | 2-(6- methyl- pyridin-2-yl) acetaldehyde | 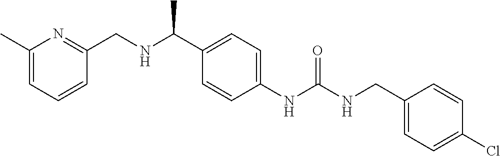 [SMILES: Cc1cccc(CN[C@@H](C)c2ccc(NC(=O)NCc3ccc(Cl)cc3)cc2)n1] |
| | | | Compound 55: {[4-((1S)-1-{[(6-methyl(2- |
| | | | pyridyl))methyl]amino}ethyl)phenyl]amino}-N-[(4- |
| | | | chlorophenyl)methyl]carboxamide. |
| | | | LCMS-ESI (POS.) m/z: 410.10 (M + H)+. |
| | | | ¹H NMR (400 MHz, DMSO-d₆) δ 8.58 (s, |
| | | | 1H), 8.18 (s, 1H), 7.62 (t, J = 7.6 Hz, 1H), |
| | | | 7.46-7.29 (m, 7H), 7.20 (t, J = 8.5 Hz, |
| | | | 3H), 7.09 (d, J = 7.6 Hz, 1H), 6.66 (s, 1H), |
| | | | 4.28 (d, J = 5.9 Hz, 2H), 3.59 (s, 2H), 2.43 |
| | | | (s, 3H), 1.29 (d, J = 6.5 Hz, 3H). |
| 56 | 2.2 | 2- cyclohexyl- acetaldehyde | 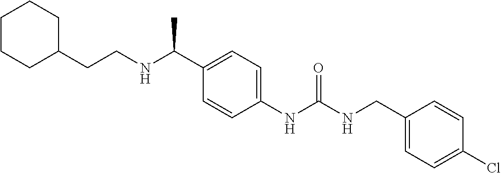 [SMILES: C[C@H](NCCC1CCCCC1)c1ccc(NC(=O)NCc2ccc(Cl)cc2)cc1] |
| | | | Compound 56: [(4-{(1S)-1- |
| | | | [(cyclohexylmethyl)amino]ethyl}phenyl)amino]-N-[(4- |
| | | | chlorophenyl)methyl]carboxamide. |
| | | | LCMS-ESI (POS.) m/z: 401.20 (M + H)+. |
| | | | ¹H NMR (400 MHz, DMSO-d₆) δ 8.70 (s, |
| | | | 1H), 8.26 (s, 1H), 7.43-7.27 (m, 5H), 7.24- |
| | | | 7.17 (m, 2H), 6.81 (t, J = 6.0 Hz, 1H), |
| | | | 4.28 (d, J = 6.0 Hz, 2H), 3.70 (t, J = 6.4 Hz, |
| | | | 1H), 2.30 (dd, J = 11.6, 6.2 Hz, 1H), 2.14 |
| | | | (dd, J = 11.6, 7.2 Hz, 1H), 1.68 (dd, J = |
| | | | 41.3, 12.7 Hz, 5H), 1.47-1.31 (m, 1H), |
| | | | 1.27 (d, J = 6.6 Hz, 3H), 1.23-1.04 (m, |
| | | | 4H), 0.90-0.71 (m, 2H). |
| 58 | 2.2 | 2-(4- methyl- pyridin-2-yl) acetaldehyde | 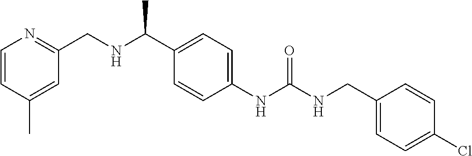 [SMILES: Cc1ccnc(CN[C@@H](C)c2ccc(NC(=O)NCc3ccc(Cl)cc3)cc2)c1] |
| | | | Compound 58: {[4-((1S)-1-{[(4-methyl(2- |
| | | | pyridyl))methyl]amino}ethyl)phenyl]amino}-N-[(4- |
| | | | chlorophenyl)methyl]carboxamide. |
| | | | LCMS-ESI (POS.) m/z: 410.10 (M + H)+. |
| | | | ¹H NMR (400 MHz, DMSO-d₆) δ 8.60 (s, |
| | | | 1H), 8.33 (d, J = 5.0 Hz, 1H), 8.18 (s, 1H), |
| | | | 7.42-7.29 (m, 5H), 7.25-7.17 (m, 3H), |
| | | | 7.08 (d, J = 5.0 Hz, 1H), 6.68 (t, J = 6.1 Hz, |
| | | | 1H), 4.28 (d, J = 5.9 Hz, 2H), 3.73 (q, J = |
| | | | 6.3 Hz, 1H), 3.60 (d, J = 3.1 Hz, 2H), 2.30 |
| | | | (s, 3H), 1.30 (d, J = 6.6 Hz, 3H). |

### Example 6

### Synthesis of N-[(4-chlorophenyl)methyl]({4-[(methylsulfonyl)methyl]phenyl}amino)carboxamide (Compound 136)

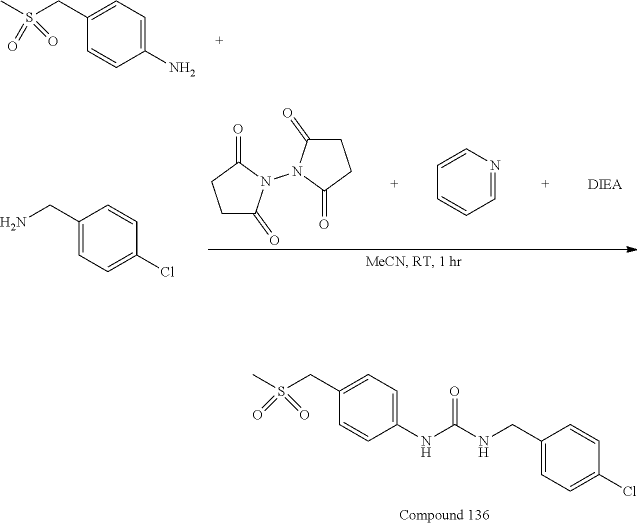 [SMILES: CS(=O)(=O)Cc1ccc(N)cc1 ; NCc1ccc(Cl)cc1 ; O=C1CCC(=O)N1N1C(=O)CCC1=O ; c1ccncc1 ; CS(=O)(=O)Cc1ccc(NC(=O)NCc2ccc(Cl)cc2)cc1]

To a room temperature solution of N,N′-disuccinimidyl carbonate (553 mg, 2.16 mmol, 1.0 equiv) in acetonitrile (10 mL) was added 4-(methanesulfonylmethyl)aniline (0.40 g, 2.16 mmol, 1.0 equiv) followed by pyridine (0.174 mL, 2.16 mmol, 1.0 equiv) in a dropwise fashion. After 20 minutes, a solution 4-chloro benzyl amine (290 mg, 2.05 mmol, 0.95 equiv) in acetonitrile (2 mL) was added followed by N,N-diisopropylethylamine (0.752 mL, 4.32 mmol, 2.0 equiv). The resulting mixture was stirred at room temperature for approximately one hour then concentrated to dryness. Resultant mixture was diluted with ethyl acetate (50 mL) and extracted with water (2×15 mL) and brine (1×15 mL). The organic phase was dried to a viscous oil which was crystallized from dichloromethane and diethyl ether. Slurry was filtered to afford N-[(4-chlorophenyl)methyl]({4-[(methylsulfonyl)methyl]phenyl}amino)carboxamide as a white solid (362 mg, 1.03 mmol, 50% yield). LCMS-APCI (POS.) m/z: 353.0 (M+H)+. 1H NMR (400 MHz, DMSO-d6) δ 8.72 (s, 1H), 7.47-7.34 (m, 4H), 7.38-7.28 (m, 2H), 7.31-7.21 (m, 2H), 6.70 (t, J=6.1 Hz, 1H), 4.36 (s, 2H), 4.29 (d, J=6.0 Hz, 2H), 2.85 (s, 3H).

Compounds in the following table were prepared in a similar manner as Compound 136, using the intermediates and reagents as listed.

| Ex# | Reagent I | Reagent II | Structure, Name and Data |
|---|---|---|---|
| 66 | 4-(pyridin-4- ylmethyl)aniline | 4-chloro benzyl amine | 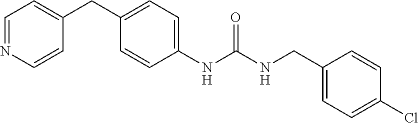 [SMILES: O=C(NCc1ccc(Cl)cc1)Nc1ccc(Cc2ccncc2)cc1] |
| | | | Compound 66: N-[(4- |
| | | | chlorophenyl)methyl]{[4-(4- |
| | | | pyridylmethyl)phenyl]amino}carboxamide. |
| | | | LCMS-ESI (POS.) m/z: 352.1 (M + H)+. 1H |
| | | | NMR (400 MHz, DMSO-d6) δ 8.56 (s, 1H), |
| | | | 8.43 (s, 2H), 7.43-7.25 (m, 6H), 7.25-7.16 |
| | | | (m, 2H), 7.13-7.04 (m, 2H), 6.62 (t, J = 6.0 |
| | | | Hz, 1H), 4.27 (d, J = 6.0 Hz, 2H), 3.87 (s, 2H). |
| 233 | 4-(pyridin-3- ylmethyl)aniline | 4-methoxy benzyl amine | 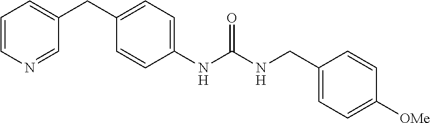 [SMILES: COc1ccc(CNC(=O)Nc2ccc(Cc3cccnc3)cc2)cc1] |
| | | | Compound 233: N-[(4- |
| | | | methoxyphenyl)methyl]{[4-(3- |
| | | | pyridylmethyl)phenyl]amino}carboxamide. |
| | | | LCMS-ESI (POS.) m/z: 348.1 (M + H)+. 1H |
| | | | NMR (400 MHz, DMSO-d6) δ 8.51-8.42 |
| | | | (m, 2H), 8.40 (dd, J = 4.8, 1.7 Hz, 1H), 7.59 |
| | | | (ddd, J = 7.8, 2.3, 1.6 Hz, 1H), 7.36-7.25 |
| | | | (m, 3H), 7.25-7.13 (m, 2H), 7.13-7.06 (m, |
| | | | 2H), 6.93-6.83 (m, 2H), 6.47 (t, J = 5.9 Hz, |
| | | | 1H), 4.20 (d, J = 5.8 Hz, 2H), 3.87 (s, 2H), |
| | | | 3.73 (s, 3H). |
| 122 | 4-[(4- methylpyrazol-1- yl)methyl]aniline | 4-chloro benzyl amine | 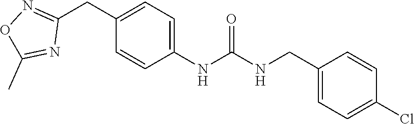 |
| | | | Compound 122: {[(4- |
| | | | chlorophenyl)methyl]amino}-N-{4-[(5- |
| | | | methyl(1,2,4-oxadiazol-3- |
| | | | yl))methyl]phenyl}carboxamide. LCMS- |
| | | | ESI (POS.) m/z: 357.2 (M + H)+. ¹H NMR |
| | | | (400 MHz, DMSO-d₆) δ 8.59 (s, 1H), 7.43- |
| | | | 7.28 (m, 6H), 7.20-7.08 (m, 2H), 6.64 (t, J = |
| | | | 6.0 Hz, 1H), 4.27 (d, J = 5.9 Hz, 2H), 3.93 (s, |
| | | | 2H), 2.53 (s, 3H). |
| 140 | 4-((3,5- dimethyl-1H- pyrazol-4- yl)methyl)aniline | 4-chloro benzyl amine | 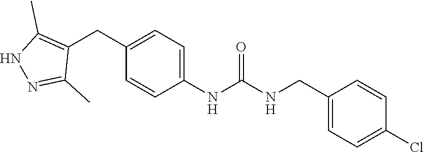 [SMILES: Cc1n[nH]c(C)c1Cc1ccc(NC(=O)NCc2ccc(Cl)cc2)cc1] |
| | | | Compound 140: N-{4-[(3,5- |
| | | | dimethylpyrazol-4-yl)methyl]phenyl}{[(4- |
| | | | chlorophenyl)methyl]amino}carboxamide. |
| | | | LCMS-ESI (POS.) m/z: 369.20 (M + H)+. ¹H |
| | | | NMR (400 MHz, DMSO-d₆) δ 8.66 (s, 1H), |
| | | | 7.42-7.35 (m, 4H), 7.33-7.22 (m, 4H), 6.96 |
| | | | (dd, J = 8.4, 6.4 Hz, 2H), 6.82-6.65 (m, 1H), |
| | | | 4.36 (d, J = 6.3 Hz, 2H), 4.26 (d, J = 5.9 Hz, |
| | | | 2H), 2.44 (s, 3H), 2.06 (s, 3H). |

### Example 7

### Synthesis of N-(4-{2-[(3S)-3-(hydroxymethyl)piperazinyl]-2-oxoethyl}phenyl){[(4-fluorophenyl)methyl]amino}carboxamide (Compound 181)

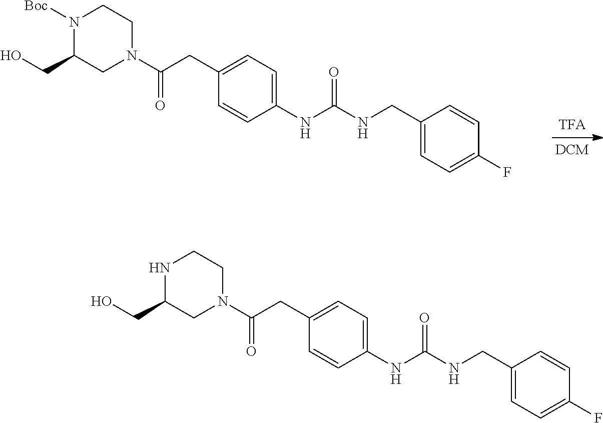 [SMILES: CC(C)(C)OC(=O)N1CCN(C(=O)Cc2ccc(NC(=O)NCc3ccc(F)cc3)cc2)C[C@H]1CO ; O=C(NCc1ccc(F)cc1)Nc1ccc(CC(=O)N2CCN[C@H](CO)C2)cc1]

2,2,2-Trifluoroacetic acid (1 mL) was added to a solution of tert-butyl (2R)-4-{2-[4-({[(4-chlorophenyl)methyl]amino}carbonylamino)phenyl]acetyl}-2-(hydroxymethyl)piperazinecarboxylate (150 mg, 0.30 mmol, 1.0 equiv) in methylene chloride (5 mL), dropwise. The resulting mixture was stirred at room temperature for approximately 3 hours. Resultant reaction mixture was dried and the resulting residue was purified by reverse phase HPLC with a 10%-100% acetonitrile in water solution that was run over 30 minutes in a Phenomonex Gemini 5u C18 column, providing the desired product (92.0 mg, 0.23 mmol, 77% yield) as a white solid. LCMS-ESI (POS.) m/z: 401.10 (M+H)+. 1H NMR (400 MHz, DMSO-d₆) δ 8.87 (s, 1H), 8.59 (s, 1H), 7.34 (t, J=7.4 Hz, 3H), 7.16 (t, J=8.7 Hz, 2H), 7.08 (d, J=8.1 Hz, 2H), 6.68 (t, J=6.0 Hz, 1H), 5.55-5.41 (m, 1H), 4.44-4.32 (m, 1H), 4.27 (d, J=5.9 Hz, 2H), 4.17-3.93 (m, 2H), 3.78-3.57 (m, 3H), 3.53 (dd, J=11.4, 5.6 Hz, 1H), 3.18 (d, J=3.8 Hz, 2H), 3.14 (s, 1H), 2.98-2.68 (m, 2H).

Compounds in the following table were prepared in a similar manner as Compound 181, using the intermediates and reagents as listed.

| | Parent | |
|---|---|---|
| Ex# | Compound | Structure, Name and Data |
| 137 | tert-butyl (2R)-4-{2-[4- ({[(4- chlorophenyl) methyl]amino} carbonylamino) phenyl] acetyl}-2- (hydroxymethyl) piperazine- carboxylate | 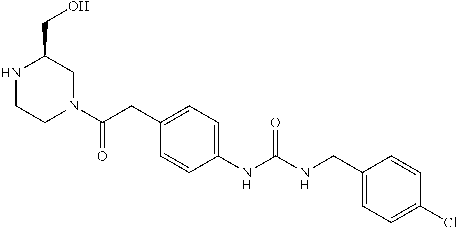 [SMILES: O=C(NCc1ccc(Cl)cc1)Nc1ccc(CC(=O)N2CCN[C@@H](CO)C2)cc1] |
| | | Compound 137: N-(4-{2-[(3R)-3- |
| | | (hydroxymethyl)piperazinyl]-2- |
| | | oxoethyl}phenyl){[(4- |
| | | chlorophenyl)methyl]amino}carboxamide. |
| | | LCMS-ESI (POS.) m/z: 418.10 |
| | | (M + H)+. ¹H NMR (400 MHz, DMSO-d₆) |
| | | δ 9.03 (s, 1H), 8.68 (s, 1H), 8.61 (s, 1H), |
| | | 7.45-7.26 (m, 5H), 7.08 (d, J = 8.2 Hz, |
| | | 2H), 6.69 (t, J = 6.0 Hz, 1H), 5.50 (s, 1H), |
| | | 4.39 (d, J = 14.3 Hz, 1H), 4.28 (d, J = 5.9 |
| | | Hz, 2H), 4.04 (dd, J = 33.0, 15.9 Hz, 1H), |
| | | 3.78-3.58 (m, 3H), 3.53 (s, 2H), 3.35- |
| | | 3.09 (m, 2H), 3.00-2.76 (m, 2H). |
| 129 | tert-butyl 6- {2-[4-({[(4- chlorophenyl) methyl]amino} carbonylamino) phenyl] acetyl}-2,6- diazaspiro[3.3] heptane-2- carboxylate | 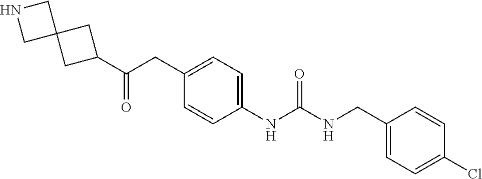 [SMILES: O=C(NCc1ccc(Cl)cc1)Nc1ccc(CC(=O)C2CC3(CNC3)C2)cc1] |
| | | Compound 129: N-{4-[2-(2,6- |
| | | diazaspiro [3.3] hept-2-yl)-2- |
| | | oxoethyl]phenyl}{[(4- |
| | | chlorophenyl)methyl]amino}carboxamide. |
| | | LCMS-ESI (POS.) m/z: 400.05 |
| | | (M + H)+. ¹H NMR (400 MHz, DMSO-d₆) |
| | | δ 8.68 (s, IH), 7.40 (d, J = 8.0 Hz, 2H), 7.36- |
| | | 7.29 (m, 4H), 7.06 (d, J = 8.1 Hz, 2H), |
| | | 6.78 (s, 1H), 4.28 (d, J = 6.9 Hz, 4H), 3.96 |
| | | (d, J = 10.4 Hz, 6H), 3.30 (s, 3H). |
| 190 | {[4-(2- {(5S,1R)-6- [(tert-butoxy) carbonylamino]- 3-azabicyclo [3.1.0]hex-3- yl}-2-oxoethyl) phenyl]amino}- N-[(4- methoxyphenyl) methyl] carboxamide | 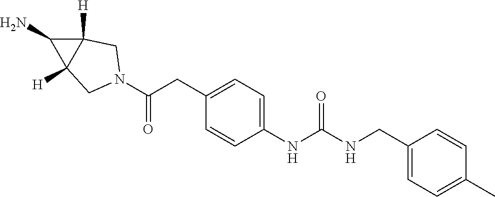 [SMILES: Cc1ccc(CNC(=O)Nc2ccc(CC(=O)N3C[C@@H]4[C@@H](N)[C@@H]4C3)cc2)cc1] |
| | | Compound 190: N-{4-[2-((5S,1R)-6- |
| | | amino-3-azabicyclo[3.1.0]hex-3-yl)-2- |
| | | oxoethyl]phenyl}{[(4- |
| | | fluorophenyl)methyl]amino}carboxamide. |
| | | LCMS-ESI (POS.) m/z: 383.10 |
| | | (M + H)+. ¹H NMR (400 MHz, Methanol- |
| | | d₄) δ 7.39-7.31 (m, 4H), 7.14 (dd, J = 8.6, |
| | | 2.1 Hz, 2H), 7.06 (td, J = 8.8, 2.1 Hz, 2H), |
| | | 5.50 (d, J = 1.9 Hz, 1H), 4.37 (s, 2H), 3.84 |
| | | (ddd, J = 13.6, 11.7, 2.2 Hz, 2H), 3.63 (d, J = |
| | | 20.1 Hz, 3H), 3.53-3.43 (m, 1H), 3.40- |
| | | 3.28 (m, 2H), 2.27 (d, J = 2.5 Hz, 1H), |
| | | 2.12-1.97 (m, 3H). |
| 90 | tert-butyl (2S)-4-{2-[4- ({[(4- chlorophenyl) methyl]amino} carbonylamino) phenyl]acetyl}- 2-methylpipera- zinecarboxylate | 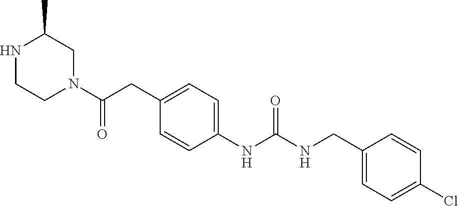 [SMILES: C[C@H]1CN(C(=O)Cc2ccc(NC(=O)NCc3ccc(Cl)cc3)cc2)CCN1] |
| | | Compound 90: N-{4-[2-((3S)-3- |
| | | methylpiperazinyl)-2- |
| | | oxoethyl]phenyl}{[(4- |
| | | chlorophenyl)methyl]amino}carboxamide. |
| | | LCMS-ESI (POS.) m/z: 401.00 |
| | | (M + H)+. ¹H NMR (400 MHz, DMSO-d₆) |
| | | δ 8.55 (s, 1H), 7.36 (dd, J = 28.2, 8.0 Hz, |
| | | 5H), 7.08 (d, J = 8.0 Hz, 2H), 6.64 (t, J = |
| | | 6.0 Hz, 1H), 4.24 (dd, J = 32.0, 8.9 Hz, 4H), |
| | | 3.67 (d, J = 61.5 Hz, 3H), 3.23-2.64 (m, |
| | | 3H), 2.47-2.09 (m, 2H), 0.91 (dd, J = |
| | | 19.0, 6.2 Hz, 3H). |
| 81 | tert-butyl (2S)-4-{2-[4- ({[(4- chlorophenyl) methyl]amino} carbonylamino) phenyl] acetyl}-2- (hydroxymethyl) piperazine- carboxylate | 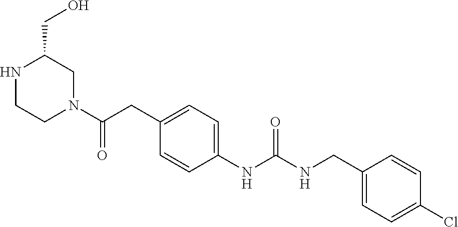 [SMILES: O=C(NCc1ccc(Cl)cc1)Nc1ccc(CC(=O)N2CCN[C@H](CO)C2)cc1] |
| | | Compound 81: N-(4-{2-[(3S)-3- |
| | | (hydroxymethyl)piperazinyl]-2- |
| | | oxoethyl}phenyl){[(4- |
| | | chlorophenyl)methyl]amino}carboxamide. |
| | | LCMS-ESI (POS.) m/z: 417.00 |
| | | (M + H)+. ¹H NMR (400 MHz, DMSO-d₆) |
| | | δ 8.71 (s, 2H), 8.59 (s, 1H), 7.40 (d, J = 8.2 |
| | | Hz, 2H), 7.34 (t, J = 9.2 Hz, 3H), 7.08 (d, |
| | | J = 8.1 Hz, 2H), 6.68 (t, J = 6.1 Hz, 1H), 5.45 |
| | | (d, J = 5.2 Hz, 1H), 4.37 (t, J = 11.1 Hz, |
| | | 1H), 4.28 (d, J = 5.9 Hz, 2H), 4.14-3.97 |
| | | (m, 1H), 3.78-3.57 (m, 3H), 3.52 (dd, J = |
| | | 11.7, 5.9 Hz, 1H), 3.29-3.04 (m, 3H), 3.01- |
| | | 2.72 (m, 2H). |
| 8 | tert-butyl 5- {2-[4-({[(4- chlorophenyl) methyl]amino} carbonylamino) phenyl] acetyl}-2,5- diazabicyclo [4.1.0]heptane- 2-carboxylate | 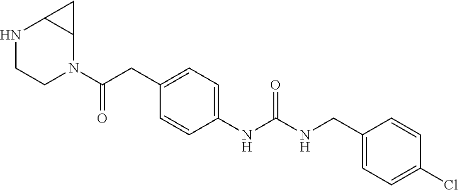 [SMILES: O=C(NCc1ccc(Cl)cc1)Nc1ccc(CC(=O)N2CCNC3CC32)cc1] |
| | | Compound 8: N-{4-[2-(2,5- |
| | | diazabicyclo[4.1.0]hept-2-yl)-2- |
| | | oxoethyl]phenyl}{[(4- |
| | | chlorophenyl)methyl]amino}carboxamide. |
| | | LCMS-ESI (POS.) m/z: 399.00 |
| | | (M + H)+. ¹H NMR (400 MHz, DMSO-d₆) |
| | | δ 8.54 (s, 1H), 7.43-7.29 (m, 6H), 7.08 |
| | | (dd, J = 15.2, 8.3 Hz, 2H), 6.62 (t, J = 6.0 |
| | | Hz, 1H), 4.28 (d, J = 5.9 Hz, 2H), 3.80 (d, |
| | | J = 15.5 Hz, 1H), 3.72-3.59 (m, 1H), 3.40 |
| | | (d, J = 5.9 Hz, 2H), 3.08 (dd, J = 24.1, 12.6 |
| | | Hz, 3H), 2.91 (s, 1H), 1.91 (s, 1H), 1.13 (q, |
| | | J = 6.5 Hz, 1H), 0.60 (d, J = 5.8 Hz, 1H). |
| 94 | tert-butyl (3R)-4-{2-[4- ({[(4- chlorophenyl) methyl]amino} carbonylamino) phenyl]acetyl}- 3-methylpipera- zinecarboxylate | 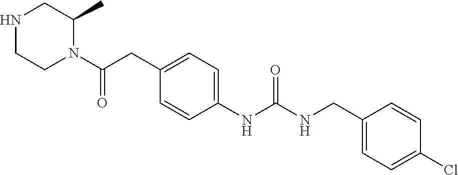 [SMILES: C[C@@H]1CNCCN1C(=O)Cc1ccc(NC(=O)NCc2ccc(Cl)cc2)cc1] |
| | | Compound 94: N-{4-[2-((2R)-2- |
| | | methylpiperazinyl)-2- |
| | | oxoethyl]phenyl}{[(4- |
| | | chlorophenyl)methyl]amino}carboxamide. |
| | | LCMS-ESI (POS.) m/z: 401.05 |
| | | (M + H)+. ¹H NMR (400 MHz, DMSO-d₆) |
| | | δ 8.66 (s, 1H), 7.43-7.29 (m, 6H), 7.07 (s, |
| | | 2H), 6.73 (s, 1H), 4.28 (d, J = 6.0 Hz, 2H), |
| | | 3.57 (s, 2H), 3.32 (s, 1H), 2.77 (s, 2H), 2.63 |
| | | (s, 2H), 2.22 (s, 2H), 1.24 (s, 1H), 1.10 (s, 3H). |
| 109 | tert-butyl (3R)-4-{2-[4- ({[(4- chlorophenyl) methyl]amino} carbonylamino) phenyl] acetyl}-3- (hydroxymethyl) piperazine- carboxylate | 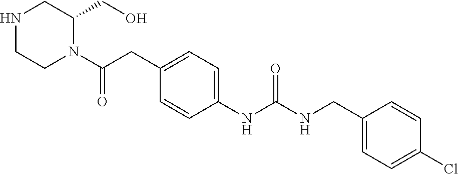 [SMILES: O=C(NCc1ccc(Cl)cc1)Nc1ccc(CC(=O)N2CCNC[C@@H]2CO)cc1] |
| | | Compound 109: N-(4-{2-[(2R)-2- |
| | | (hydroxymethyl)piperazinyl]-2- |
| | | oxoethyl}phenyl){[(4- |
| | | chlorophenyl)methyl]amino}carboxamide. |
| | | LCMS-ESI (POS.) m/z: 417.00 |
| | | (M + H)+. ¹H NMR (400 MHz, DMSO-d₆) |
| | | δ 8.55 (s, 1H), 7.46-7.21 (m, 6H), 7.07 (d, |
| | | J = 8.1 Hz, 2H), 6.63 (t, J = 6.1 Hz, 1H), |
| | | 5.04 (s, 1H), 4.87 (s, 1H), 4.59-4.05 (m, |
| | | 5H), 3.87 (ddd, J = 46.7, 31.3, 14.5 Hz, |
| | | 3H), 3.71-3.54 (m, 2H), 3.25-3.12 (m, |
| | | 1H), 2.98 (d, J = 12.0 Hz, 1H), 2.87 (s, 1H). |
| 101 | tert-butyl 8- {2-[4-({[(4- chlorophenyl) methyl]amino} carbonylamino) phenyl] acetyl}-3,8- diazabicyclo [3.2.1]octane- 3-carboxylate | 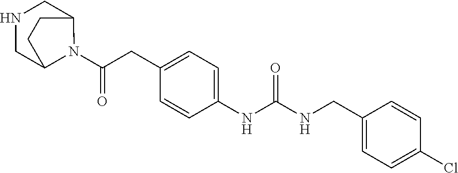 [SMILES: O=C(NCc1ccc(Cl)cc1)Nc1ccc(CC(=O)N2C3CCC2CNC3)cc1] |
| | | Compound 101: N-{4-[2-(3,8- |
| | | diazabicyclo[3.2.1]oct-8-yl)-2- |
| | | oxoethyl]phenyl}{[(4- |
| | | chlorophenyl)methyl]amino}carboxamide. |
| | | LCMS-ESI (POS.) m/z: 413.05 |
| | | (M + H)+. ¹H NMR (400MHz, _{1,4}-Dioxane- |
| | | d₈) δ 9.64 (s, 3H), 8.50-8.33 (m, 5H), 8.15 |
| | | (d, J = 8.1 Hz, 2H), 7.72 (t, J = 6.1 Hz, 1H), |
| | | 5.62 (s, 2H), 5.32 (d, J = 5.9 Hz, 2H), 4.74- |
| | | 4.55 (m, 2H), 4.22 (d, J = 5.0 Hz, 1H), |
| | | 4.12 (dd, J = 18.1, 12.5 Hz, 2H), 3.99 (d, |
| | | J = 12.3 Hz, 1H), 3.90 (d, J = 12.5 Hz, 1H), |
| | | 2.93 (d, J = 7.7 Hz, 4H). |
| 59 | tert-butyl (3S,5R)-4- {2-[4-({[(4- chlorophenyl) methyl]amino} carbonylamino) phenyl] acetyl}-3,5- dimethylpipera- zinecarboxylate | 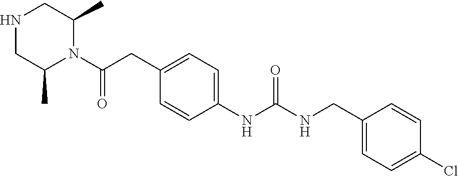 [SMILES: C[C@@H]1CNC[C@H](C)N1C(=O)Cc1ccc(NC(=O)NCc2ccc(Cl)cc2)cc1] |
| | | Compound 59: tert-butyl (3S,5R)-4-{2- |
| | | [4-({[(4-chlorophenyl)methyl]amino} |
| | | carbonylamino)phenyl]acetyl}-3,5- |
| | | dimethylpiperazinecarboxylate. LCMS- |
| | | ESI (POS.) m/z: 415.10 (M + H)+. ¹H NMR |
| | | (400 MHz, Methanol-d₄) δ 7.23 (q, J = 9.2, |
| | | 8.7 Hz, 6H), 7.07 (d, J = 8.3 Hz, 2H), 4.27 |
| | | (s, 2H), 3.66 (s, 2H), 3.04 (d, J = 12.1 Hz, |
| | | 2H), 3.30 (d, J = 12.1 Hz, 2H), 2.92 (d, J = |
| | | 9.3 Hz, 2H), 1.21 (d, J = 7.3 Hz, 6H). |
| 42 | tert-butyl (3S)-4-{2-[4- ({[(4- chlorophenyl) methyl]amino} carbonylamino) phenyl] acetyl}-3- methylpipera- zinecarboxylate | 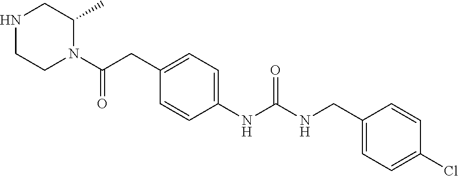 [SMILES: C[C@H]1CNCCN1C(=O)Cc1ccc(NC(=O)NCc2ccc(Cl)cc2)cc1] |
| | | Compound 42: N-{4-[2-((2S)-2- |
| | | methylpiperazinyl)-2- |
| | | oxoethyl]phenyl}{[(4- |
| | | chlorophenyl)methyl]amino}carboxamide. |
| | | LCMS-ESI (POS.) m/z: 401.00 |
| | | (M + H)+. ¹H NMR (400 MHz, DMSO-d₆) δ |
| | | 8.55 (s, 1H), 7.56-7.21 (m, 6H), 7.12- |
| | | 6.96 (m, 2H), 6.65 (t, J = 6.0 Hz, 1H), 4.44 |
| | | (s, 1H), 4.28 (s, 2H), 4.10 (d, J = 13.1 Hz, |
| | | 1H), 4.00 (s, 1H), 3.58 (d, J = 15.9 Hz, 2H), |
| | | 3.05-2.94 (m, 1H), 2.85-2.72 (m, 1H), |
| | | 2.69-2.53 (m, 2H), 2.32 (q, J = 12.9 Hz, |
| | | 1H), 1.09 (d, J = 6.7 Hz, 3H). |
| 142 | tert-butyl 3- {2-[4-({N-[(4- chlorophenyl) methyl] carbamoyl} amino)phenyl] acetylamino} azetidine- carboxylate | 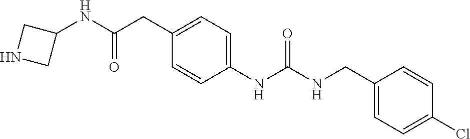 [SMILES: O=C(Cc1ccc(NC(=O)NCc2ccc(Cl)cc2)cc1)NC1CNC1] |
| | | Compound 142: N-azetidin-3-yl-2-[4-({[(4- |
| | | chlorophenyl)methyl]amino}carbonylamino) |
| | | phenyl]acetamide. LCMS-ESI (POS.) |
| | | m/z: 373.00 (M + H)+. ¹H NMR (400 MHz, |
| | | DMSO-d₆) δ 9.06 (s, 1H), 8.56 (d, |
| | | J = 7.4 Hz, 1H), 7.38 (d, J = 8.1 Hz, 2H), 7.32 |
| | | (d, J = 7.9 Hz, 3H), 7.09 (d, J = 7.8 Hz, 3H), |
| | | 4.44 (h, J = 7.4 Hz, 1H), 4.27 (d, J = 5.9 |
| | | Hz, 2H), 3.52 (t, J = 7.6 Hz, 2H), 3.29 (s, |
| | | 3H), 1.78 (s, 1H). |
| 146 | tert-butyl 5- {2-[4-({[(4- chlorophenyl) methyl]amino} carbonylamino) phenyl] acetyl}-2,5- diazabicyclo [2.2.1]heptane- 2-carboxylate | 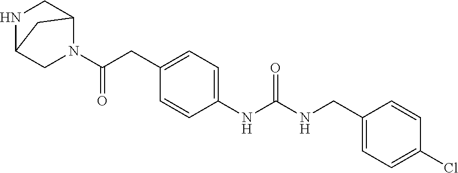 [SMILES: O=C(NCc1ccc(Cl)cc1)Nc1ccc(CC(=O)N2CC3CC2CN3)cc1] |
| | | Compound 146: N-{4-[2-(2,5- |
| | | diazabicyclo[2.2.1]hept-2-yl)-2- |
| | | oxoethyl]phenyl}{[(4- |
| | | chlorophenyl)methyl]amino}carboxamide. |
| | | LCMS-ESI (POS.) m/z: 399.00 |
| | | (M + H)+. ¹H NMR (400 MHz, DMSO-d₆) δ |
| | | 8.61 (d, J = 5.3 Hz, 1H), 7.39 (d, J = 8.1 |
| | | Hz, 2H), 7.33 (s, 2H), 7.31 (s, 2H), 7.08 |
| | | (dd, J = 15.8, 8.1 Hz, 2H), 6.71 (q, J = 5.5 |
| | | Hz, 1H), 4.55 (s, 1H), 4.28 (d, J = 5.9 Hz, |
| | | 2H), 4.11 (s, 1H), 3.69 (d, J = 24.3 Hz, 1H), |
| | | 3.26-3.06 (m, 3H), 2.84 (t, J = 10.5 Hz, |
| | | 1H), 1.85 (s, 1H), 1.61 (dq, J = 21.5, 9.7 |
| | | Hz, 2H), 1.24 (s, 1H). |
| 29 | tert-butyl (2R)-4-{2-[4- ({[(4- chlorophenyl) methyl]amino} carbonylamino) phenyl] acetyl}-2- (methoxy- methyl) piperazine- carboxylate | 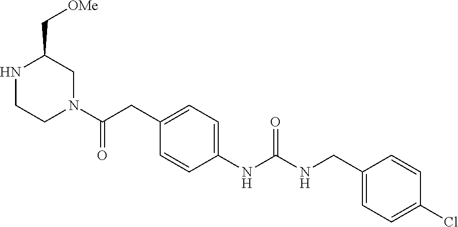 [SMILES: COC[C@H]1CN(C(=O)Cc2ccc(NC(=O)NCc3ccc(Cl)cc3)cc2)CCN1] |
| | | Compound 29: [(4-{2-[(3R)-3- |
| | | (methoxymethyl)piperazinyl]-2- |
| | | oxoethyl}phenyl)amino]-N-[(4- |
| | | chlorophenyl)methyl]carboxamide. |
| | | LCMS-ESI (POS.) m/z: 431.20 (M + H)+. |
| | | ¹H NMR (400 MHz, DMSO-d₆, 1:1 ratio of |
| | | rotamers) δ 8.58 (s, 2H), 7.40 (d, J = 8.0 |
| | | Hz, 4H), 7.36-7.30 (m, 8H), 7.07 (d, J = |
| | | 8.1 Hz, 4H), 6.66 (t, J = 6.2 Hz, 2H), 4.28 |
| | | (d, J = 6.2 Hz, 5H), 4.21 (d, J = 13.0 Hz, |
| | | 1H), 3.85 (dd, J = 18.5, 13.4 Hz, 2H), 3.62 |
| | | (s, 5H), 3.32-3.28 (m, 5H), 3.27 (s, 3H), |
| | | 3.24 (s, 3H), 3.04 (t, J = 12.1 Hz, 2H), 2.95 |
| | | (d, J = 11.9 Hz, 1H), 2.90-2.84 (m, 2H), |
| | | 2.82-2.72 (m, 1H), 2.68 (d, J = 12.5 Hz, |
| | | 1H), 2.59 (d, J = 11.8 Hz, 1H), 2.47-2.34 |
| | | (m, 2H). |
| 33 | tert-butyl (2S)-4-{2-[4- ({[(4- chlorophenyl) methyl]amino} carbonylamino) phenyl] acetyl}-2- (methoxy- methyl) piperazine- carboxylate | 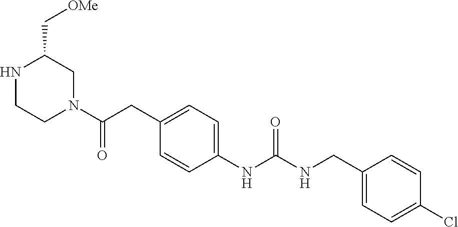 [SMILES: COC[C@@H]1CN(C(=O)Cc2ccc(NC(=O)NCc3ccc(Cl)cc3)cc2)CCN1] |
| | | Compound 33: [(4-{2-[(3S)-3- |
| | | (methoxymethyl)piperazinyl]-2- |
| | | oxoethyl}phenyl)amino]-N-[(4- |
| | | chlorophenyl)methyl]carboxamide. |
| | | LCMS-ESI (POS.) m/z: 431.20 (M + H)+. |
| | | ¹H NMR (400 MHz, DMSO-d₆, 1:1 ratio of |
| | | rotamers) δ 8.58 (s, 2H), 7.40 (d, J = 8.0 |
| | | Hz, 4H), 7.36-7.30 (m, 8H), 7.07 (d, J = |
| | | 8.1 Hz, 4H), 6.66 (t, J = 6.2 Hz, 2H), 4.28 |
| | | (d, J = 6.2 Hz, 5H), 4.21 (d, J = 13.0 Hz, |
| | | 1H), 3.85 (dd, J = 18.5, 13.4 Hz, 2H), 3.62 |
| | | (s, 5H), 3.32-3.28 (m, 5H), 3.27 (s, 3H), |
| | | 3.24 (s, 3H), 3.04 (t, J = 12.1 Hz, 2H), 2.95 |
| | | (d, J = 11.9 Hz, 1H), 2.90-2.84 (m, 2H), |
| | | 2.82-2.72 (m, 1H), 2.68 (d, J = 12.5 Hz, |
| | | 1H), 2.59 (d, J = 11.8 Hz, 1H), 2.47-2.34 |
| | | (m, 2H). |
| 377 | tert-butyl 4- (4-(3-(4- chlorobenzyl) ureido) benzyl)-3- oxopiperazine- 1-carboxylate | 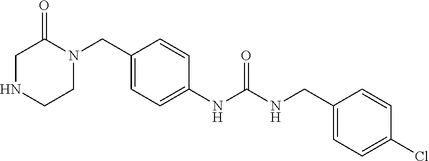 [SMILES: O=C(NCc1ccc(Cl)cc1)Nc1ccc(CN2CCNCC2=O)cc1] |
| | | 1-(4-chlorobenzyl)-3-(4-((2- |
| | | oxopiperazin-1-yl)methyl)phenyl)urea. |
| | | LCMS-ESI (POS.) m/z: 373 (M + H)⁺. |
| 396 & 549 | tert-butyl 2- (4-(3-(4- chlorobenzyl) ureido)phenyl)- 4-methyl-5- oxopiperazine- 1-carboxylate | 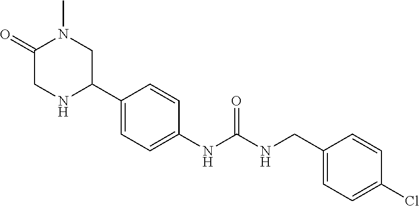 [SMILES: CN1CC(c2ccc(NC(=O)NCc3ccc(Cl)cc3)cc2)NCC1=O] |
| | | 1-(4-chlorobenzyl)-3-(4-(4-methyl-5- |
| | | oxopiperazin-2-yl)phenyl)urea. LCMS- |
| | | ESI (POS.) m/z: 373 (M + H)⁺. |
| 427 & 428 | tert-butyl 4- (4-(3-(4- chlorobenzyl) ureido)benz yl)-2-methyl-5- oxopiperazine- 1-carboxylate | 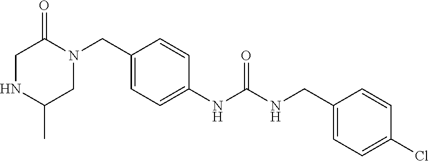 [SMILES: CC1CN(Cc2ccc(NC(=O)NCc3ccc(Cl)cc3)cc2)C(=O)CN1] |
| | | 1-(4-chlorobenzyl)-3-(4-((5-methyl-2- |
| | | oxopiperazin-1-yl)methyl)phenyl)urea. |
| | | LCMS-APCI (POS.) m/z: 387 (M + H)⁺. |
| 429 & 430 | tert-butyl 4- (4-(3-(4- chlorobenzyl) ureido)benzyl)- 3-methyl-5- oxopiperazine- 1-carboxylate | 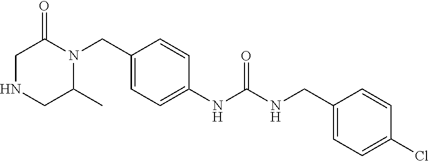 [SMILES: CC1CNCC(=O)N1Cc1ccc(NC(=O)NCc2ccc(Cl)cc2)cc1] |
| | | 1-(4-chlorobenzyl)-3-(4-((2-methyl-6- |
| | | oxopiperazin-1-yl)methyl)phenyl)urea. |
| | | LCMS-APCI (POS.) m/z: 387 (M + H)⁺. |

### Example 8

### Synthesis of N-{4-[(1S)-1-(methylsulfonyl)ethyl]phenyl}{[(4-chlorophenyl)methyl]amino}carboxamide (Compound 86) and N-{4-[(1R)-1-(methylsulfonyl)ethyl]phenyl}{[(4-chlorophenyl)methyl]amino}carboxamide (Compound 127)

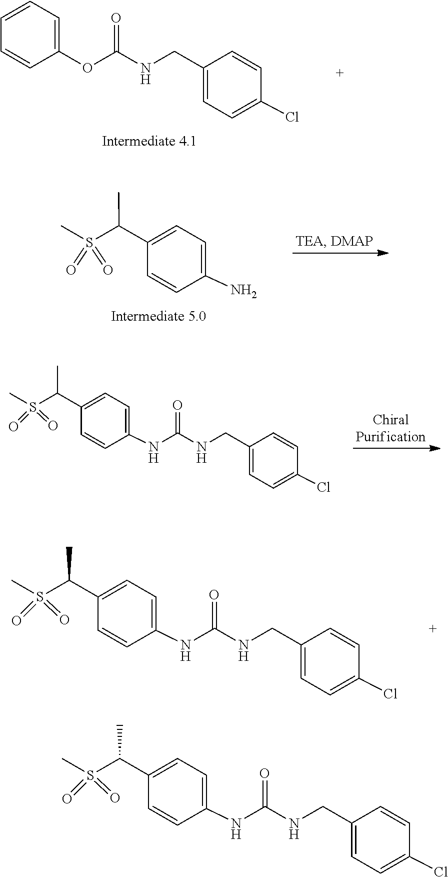 [SMILES: O=C(NCc1ccc(Cl)cc1)Oc1ccccc1 ; CC(c1ccc(N)cc1)S(C)(=O)=O ; CC(c1ccc(NC(=O)NCc2ccc(Cl)cc2)cc1)S(C)(=O)=O ; C[C@@H](c1ccc(NC(=O)NCc2ccc(Cl)cc2)cc1)S(C)(=O)=O ; C[C@H](c1ccc(NC(=O)NCc2ccc(Cl)cc2)cc1)S(C)(=O)=O]

### Step 1: Preparation of N-{4-[-1-(methylsulfonyl)ethyl]phenyl}{[(4-chlorophenyl)methyl]amino}carboxamide

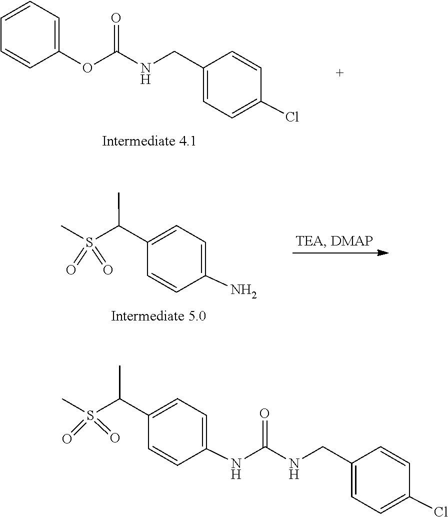 [SMILES: O=C(NCc1ccc(Cl)cc1)Oc1ccccc1 ; CC(c1ccc(N)cc1)S(C)(=O)=O ; CC(c1ccc(NC(=O)NCc2ccc(Cl)cc2)cc1)S(C)(=O)=O]

To a stirred solution of 4-(1-methanesulfonylethyl)aniline (300.00 mg, 1.505 mmol, 1.00 equiv) and phenyl N-[(4-chlorophenyl)methyl]carbamate (472.80 mg, 1.807 mmol, 1.20 equiv) in acetonitrile/THF (4 mL/2 mL) was added TEA (457.02 mg, 4.516 mmol, 3.00 equiv) at r.t. The resulting mixture was stirred at 60° C. for overnight, then concentrated under reduced pressure, purified by Prep-HPLC with the following conditions (Column: XBridge Prep OBD C18 Column 30×150 mm 5 um; Mobile Phase A: Water(10 MMOL/L NH4HCO3), Mobile Phase B: ACN; Flow rate: 60 mL/min; Gradient: 35% B to 35% B in 10 min; 254 nm; Rt: 9.68 min) to afford 3-[(4-chlorophenyl)methyl]-1-[4-(1-methanesulfonylethyl)phenyl]urea (90 mg, 16.30%) as a white solid. LRMS (ES) m/z 367[M+H].

### Step 2: Preparation of N-{4-[(1S)-1-(methylsulfonyl)ethyl]phenyl}{[(4-chlorophenyl)methyl]amino}carboxamide (Compound 86) and N-{4-[(1R)-1-(methylsulfonyl)ethyl]phenyl}{[(4-chlorophenyl)methyl]amino}carboxamide (Compound 127)

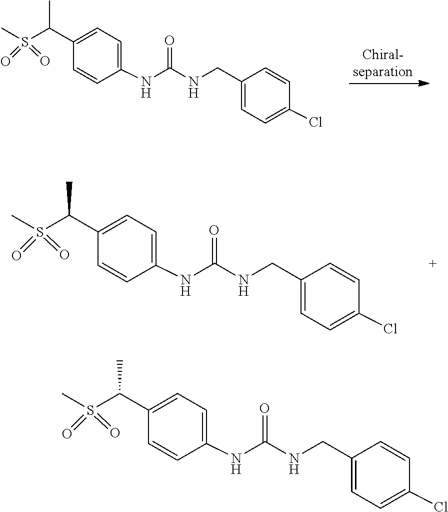 [SMILES: CC(c1ccc(NC(=O)NCc2ccc(Cl)cc2)cc1)S(C)(=O)=O ; C[C@@H](c1ccc(NC(=O)NCc2ccc(Cl)cc2)cc1)S(C)(=O)=O ; C[C@H](c1ccc(NC(=O)NCc2ccc(Cl)cc2)cc1)S(C)(=O)=O]

The racemic compound 3-[(4-chlorophenyl)methyl]-1-[4-(1-methanesulfonylethyl)phenyl]urea(90 mg, 0.257 mmol, 1.00 equiv) was separated by Chiral-HPLC with the following conditions(Column: CHIRALPAK IA, 2*25 cm, 5 um; Mobile Phase A: Hex(8 mmol/L NH3.MeOH)-HPLC, Mobile Phase B: EtOH-HPLC; Flow rate: 16 mL/min; Gradient: 50 B to 50 B in 20 min; 220/254 nm) to afford 33.9 mg 3-[(4-chlorophenyl)methyl]-1-[4-[(1S)-1-methanesulfonylethyl]phenyl]urea and 39.9 mg 3-[(4-chlorophenyl)methyl]-1-[4-[(1R)-1-methanesulfonylethyl]phenyl]urea as white solids. The chiral analytical data shows retention times of (RT: 10.53 min) and (RT: 15.92 min) for the first and second peak respectively. The first peak was arbitrarily assigned as (S)-1-(4-chlorobenzyl)-3-(4-(1-(methylsulfonyl)ethyl)phenyl)urea and second peak was assigned as (R)-1-(4-chlorobenzyl)-3-(4-(1-(methylsulfonyl)ethyl)phenyl)urea. Enantiomer 1: LRMS (ES) m/z 367 [M+H]. 1H NMR (400 MHz, DMSO-d6) δ 8.74 (s, 1H), 7.41 (dd, J=12.3, 8.2 Hz, 4H), 7.31 (t, J=8.1 Hz, 4H), 6.71 (t, J=6.0 Hz, 1H), 4.42 (q, J=7.1 Hz, 1H), 4.29 (d, J=5.9 Hz, 2H), 2.77 (s, 3H), 1.59 (d, J=7.1 Hz, 3H). Enantiomer 2: LRMS (ES) m/z 367[M+H]. 1H NMR (400 MHz, DMSO-d6) δ 8.74 (s, 1H), 7.46-7.36 (m, 4H), 7.31 (t, J=8.5 Hz, 4H), 6.71 (t, J=6.0 Hz, 1H), 4.42 (q, J=7.1 Hz, 1H), 4.29 (d, J=5.9 Hz, 2H), 2.77 (s, 3H), 1.59 (d, J=7.1 Hz, 3H).

Compounds in the following table were prepared in a similar manner as Compounds 86 and 127, using the intermediates and reagents as listed.

| | Intermediate | Intermediate | |
|---|---|---|---|
| Ex# | I | II | Structure, Name and Data |
| 80 | 4.1 | 6.0 | 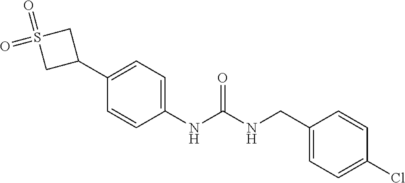 [SMILES: O=C(NCc1ccc(Cl)cc1)Nc1ccc(C2CS(=O)(=O)C2)cc1] |
| | | | Compound 80: {[4-(1,1-dioxothietan-3- |
| | | | yl)phenyl]amino}-N-[(4- |
| | | | chlorophenyl)methyl]carboxamide. |
| | | | LCMS-ESI (POS.) m/z: 365.0 (M + H)+. ¹H |
| | | | NMR (400 MHz, DMSO-d₆) δ 8.66 (s, 1H), |
| | | | 7.44-7.36 (m, 4H), 7.40-7.23 (m, 4H), |
| | | | 6.68 (t, J = 6.1 Hz, 1H), 4.60-4.49 (m, 2H), |
| | | | 4.31-4.15 (m, 4H), 3.79 (tt, J = 9.7, 7.9 Hz, 1H). |
| 313 | 4.2 | 6.0 | 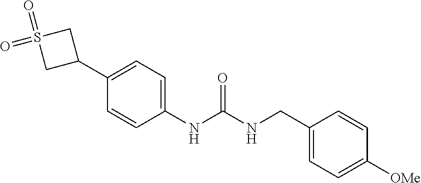 [SMILES: COc1ccc(CNC(=O)Nc2ccc(C3CS(=O)(=O)C3)cc2)cc1] |
| | | | Compound 313: {[4-(1,1-dioxothietan-3- |
| | | | yl)phenyl]amino}-N-[(4- |
| | | | methoxyphenyl)methyl]carboxamide. |
| | | | LCMS-ESI (POS.) m/z: 361.0 (M + H)+. |
| | | | ¹HNMR (400 MHz, DMSO-d₆) δ 8.55 (s, 1H), |
| | | | 7.48-7.35 (m, 2H), 7.34-7.19 (m, 4H), 6.94- |
| | | | 6.84 (m, 2H), 6.52 (t, J = 5.9 Hz, 1H), 4.60- |
| | | | 4.48 (m, 2H), 4.28-4.17 (m, 4H), 3.85-3.75 |
| | | | (m, 1H), 3.73 (s, 3H). |
| 50 | 4.1 | 9.0 | 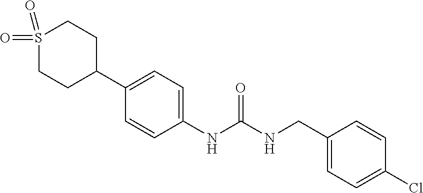 [SMILES: O=C(NCc1ccc(Cl)cc1)Nc1ccc(C2CCS(=O)(=O)CC2)cc1] |
| | | | Compound 50: {[4-(1,1-dioxothian-4- |
| | | | yl)phenyl]amino}-N-[(4- |
| | | | chlorophenyl)methyl]carboxamide. LCMS- |
| | | | ESI (POS.) m/z: 393.0 (M + H)+. ¹HNMR (400 |
| | | | MHz, DMSO-d6) δ 8.57 (s, 1H), 7.39 (d, J = 7.6 |
| | | | Hz, 2H), 7.34 (d, J = 9.0 Hz, 4H), 7.11 (d, J = |
| | | | 8.2 Hz, 2H), 6.65 (d, J = 6.6 Hz, 1H), 4.28 (d, J = |
| | | | 5.9 Hz, 2H), 3.30 (s, 2H), 3.09 (d, J = 13.7 Hz, |
| | | | 2H), 2.88-2.82 (m, 1H), 2.05 (d, J = 8.7 Hz, 4H). |
| 270 | 4.2 | 9.0 | 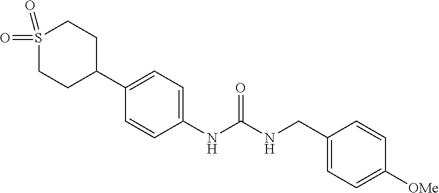 [SMILES: COc1ccc(CNC(=O)Nc2ccc(C3CCS(=O)(=O)CC3)cc2)cc1] |
| | | | Compound 270: {[4-(1,1-dioxothian-4- |
| | | | yl)phenyl]amino}-N-[(4- |
| | | | methoxyphenyl)methyl]carboxamide. |
| | | | LCMS-ESI (POS.) m/z: 389.0 (M + H)+. |
| | | | ¹HNMR (400 MHz, DMSO-d₆) δ 8.45 (s, 1H), |
| | | | 7.44-7.30 (m, 2H), 7.30-7.19 (m, 2H), 7.11 |
| | | | (d, J = 8.5 Hz, 2H), 6.93-6.85 (m, 2H), 6.49 (t, |
| | | | J = 5.8 Hz, 1H), 4.21 (d, J = 5.8 Hz, 2H), 3.73 |
| | | | (s, 3H), 3.13-3.03 (m, 2H), 2.89-2.78 (m, |
| | | | 1H), 2.04 (dq, J = 14.6, 7.9 Hz, 4H), 1.25 (s, |
| | | | 2H), |
| 78 | 4.1 | 10.0 | 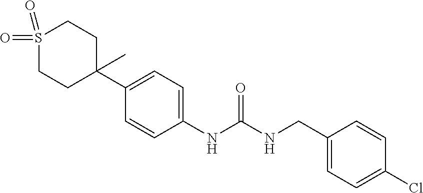 [SMILES: CC1(c2ccc(NC(=O)NCc3ccc(Cl)cc3)cc2)CCS(=O)(=O)CC1] |
| | | | Compound 78: N-[(4- |
| | | | chlorophenyl)methyl]{[4-(4-methyl-1,1- |
| | | | dioxothian-4-yl)phenyl]amino}carboxamide. |
| | | | LCMS-ESI (POS.) m/z: 407.0 (M + H)+. ¹H |
| | | | NMR (300 MHz, DMSO-d6) δ 8.63 (s, 1H), |
| | | | 7.46-7.37 (m, 4H), 7.32 (t, J = 8.4 Hz, 4H), |
| | | | 4.30 (d, J = 5.9 Hz, 2H), 3.13 (s, 2H), 2.81 (s, |
| | | | 2H), 2.06 (s, 2H), 1.23 (s, 3H). |
| 296 | 4.2 | 10.0 | 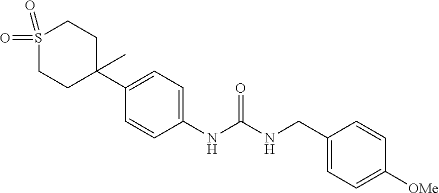 [SMILES: COc1ccc(CNC(=O)Nc2ccc(C3(C)CCS(=O)(=O)CC3)cc2)cc1] |
| | | | Compound 296: N-[(4- |
| | | | methoxyphenyl)methyl]{[4-(4-methyl-1,1- |
| | | | dioxothian-4- |
| | | | yl)phenyl]amino}carboxamide. LCMS- |
| | | | ESI (POS.) m/z: 403.0 (M + H)+. ¹H NMR |
| | | | (400 MHz, DMSO-d6) δ 8.53 (s, 1H), 7.40 |
| | | | (d, J = 8.6 Hz, 2H), 7.30 (d, J = 8.6 Hz, 2H), |
| | | | 7.23 (d, J = 8.5 Hz, 2H), 6.90 (d, J = 8.6 Hz, |
| | | | 2H), 6.51 (t, J = 6.0 Hz, 1H), 4.23 (d, J = 5.8 |
| | | | Hz, 2H), 3.74 (s, 3H), 3.14 (s, 2H), 2.81 (t, |
| | | | J = 12.6 Hz, 2H), 2.09 (s, 0H), 2.05 (s, 2H), |
| | | | 1.23 (s, 3H). |
| 302 | 4.2 | 5.0 | 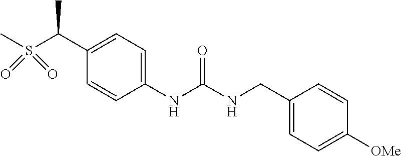 [SMILES: COc1ccc(CNC(=O)Nc2ccc([C@H](C)S(C)(=O)=O)cc2)cc1] |
| | | | Compound 302: N-{4-[(1S)-1- |
| | | | (methylsulfonyl)ethyl]phenyl}{[(4- |
| | | | methoxyphenyl)methyl]amino}carboxamide. |
| | | | LCMS-ESI (POS.) m/z: 407.0 (M + HCOO)+. |
| | | | ¹H NMR (400 MHz, DMSO-d₆) δ 8.61 (s, 1H), |
| | | | 7.40 (d, J = 8.1 Hz, 2H), 7.29 (d, J = 8.2 Hz, |
| | | | 2H), 7.22 (d, J = 8.1 Hz, 2H), 6.88 (d, J = 8.1 |
| | | | Hz, 2H), 6.55 (t, J = 5.8 Hz, 1H), 4.41 (q, J = |
| | | | 7.4 Hz, 1H), 4.21 (d, J = 5.8 Hz, 2H), 3.72 (s, |
| | | | 3H), 2.75 (s, 3H), 1.58 (d,J = 7.1 Hz, 3H). |
| 345 | 4.2 | 5.0 | 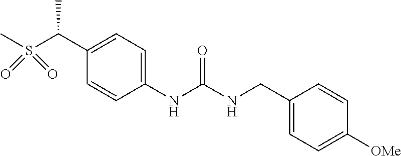 [SMILES: COc1ccc(CNC(=O)Nc2ccc([C@@H](C)S(C)(=O)=O)cc2)cc1] |
| | | | Compound 345: N-{4-[(1R)-1- |
| | | | (methylsulfonyl)ethyl]phenyl}{[(4- |
| | | | methoxyphenyl)methyl]amino}carboxamide. |
| | | | LCMS-ESI (POS.) m/z: 407.0 (M + HCOO)+. |
| | | | ¹H NMR (400 MHz, DMSO-d₆) δ 8.62 (s, 1H), |
| | | | 7.41 (d, J = 8.2 Hz, 2H), 7.29 (d, J = 8.3 Hz, |
| | | | 2H), 7.22 (d, J = 8.2 Hz, 2H), 6.89 (d, J = 8.2 |
| | | | Hz, 2H), 6.56 (t, J = 5.9 Hz, 1H), 4.41 (q, J = |
| | | | 7.1 Hz, 1H), 4.21 (d, J = 5.8 Hz, 2H), 3.72 (s, |
| | | | 3H), 2.75 (s, 3H), 1.59 (d, J = 7.1 Hz, 3H). |
| 141 | 4.1 | 7.0 | 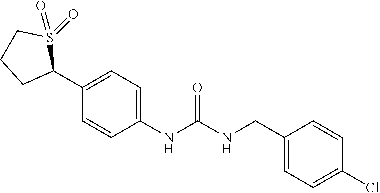 [SMILES: O=C(NCc1ccc(Cl)cc1)Nc1ccc([C@H]2CCCS2(=O)=O)cc1] |
| | | | Compound 141: {[4-((2R)-1,1-dioxothiolan- |
| | | | 2-yl)phenyl]amino}-N-[(4- |
| | | | chlorophenyl)methyl]carboxamide. LCMS- |
| | | | ESI (POS.) m/z: 379.0 (M + H)+. 1H NMR (400 |
| | | | MHz, Methanol-d4) δ 7.48-7.40 (m, 2H), 7.39- |
| | | | 7.29 (m, 6H), 4.39 (s, 2H), 4.27 (dd, J = 12.0, |
| | | | 7.1 Hz, 1H), 3.29 (dd, J = 9.3, 3.5 Hz, 1H), 3.17 |
| | | | (dt, J = 13.4, 8.9 Hz, 1H), 2.55-2.39 (m, 2H), |
| | | | 2.39-2.20 (m, 2H). |
| 148 | 4.1 | 7.0 | 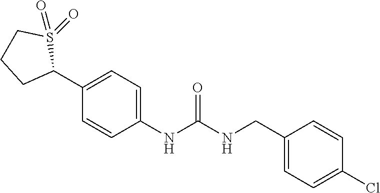 [SMILES: O=C(NCc1ccc(Cl)cc1)Nc1ccc([C@@H]2CCCS2(=O)=O)cc1] |
| | | | Compound 148: {[4-((2S)-1,1-dioxothiolan- |
| | | | 2-yl)phenyl] amino}-N-[(4- |
| | | | chlorophenyl)methyl]carboxamide. LCMS- |
| | | | ESI (POS.) m/z: 379.0 (M + H)+. 1H NMR (400 |
| | | | MHz, Methanol-d4) δ 7.48-7.40 (m, 2H), |
| | | | 7.39-7.29 (m, 6H), 4.39 (s, 2H), 4.27 (dd, J = |
| | | | 12.1, 7.1 Hz, 1H), 3.29 (dd, J = 9.3, 3.6 Hz, |
| | | | 1H), 3.17 (dt, J = 13.5, 8.8 Hz, 1H), 2.55- |
| | | | 2.43 (m, 2H), 2.46-2.20 (m, 2H). |
| 98 | 4.1 | 8.0 | 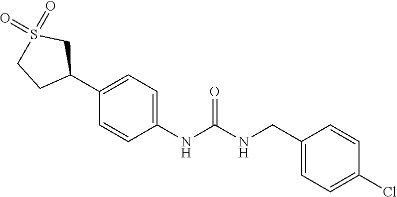 [SMILES: O=C(NCc1ccc(Cl)cc1)Nc1ccc([C@H]2CCS(=O)(=O)C2)cc1] |
| | | | Compound 98: {[4-((3R)-1,1-dioxothiolan-3- |
| | | | yl)phenyl]amino}-N-[(4- |
| | | | chlorophenyl)methyl]carboxamide. LCMS- |
| | | | ESI (POS.) m/z: 379.0 (M + H)+. ¹H NMR (400 |
| | | | MHz, DMSO-d₆) δ 8.65 (s, 1H), 7.43-7.34 (m, |
| | | | 4H), 7.32 (d, J = 8.5 Hz, 2H), 7.26-7.19 (m, |
| | | | 2H), 6.69 (t, J = 6.0 Hz, 1H), 4.28 (d, J = 6.0 |
| | | | Hz, 2H), 3.49 (ddd, J = 12.8, 6.6, 2.6 Hz, 2H), |
| | | | 3.35-3.31 (m, 1H), 3.23-3.11 (m, 1H), 3.11- |
| | | | 3.00 (m, 1H), 2.42 (ddt, J = 10.4, 8.6, 3.3 Hz, |
| | | | 1H), 2.20-2.06 (m, 1H) |
| 79 | 4.1 | 8.0 | 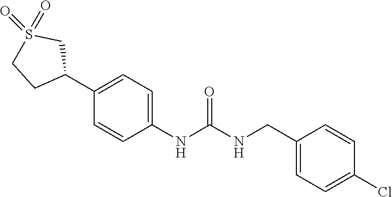 [SMILES: O=C(NCc1ccc(Cl)cc1)Nc1ccc([C@@H]2CCS(=O)(=O)C2)cc1] |
| | | | Compound 79: {[4-((3S)-1,1-dioxothiolan-3- |
| | | | yl)phenyl]amino}-N-[(4- |
| | | | chlorophenyl)methyl]carboxamide. LCMS- |
| | | | ESI (POS.) m/z: 379.0 (M + H)+. ¹H NMR (400 |
| | | | MHz, DMSO-d₆) δ 8.64 (s, 1H), 7.43-7.28 (m, |
| | | | 6H), 7.22 (d, J = 8.5 Hz, 2H), 6.68 (t, J = 6.0 |
| | | | Hz, 1H), 4.28 (d, J = 6.0 Hz, 2H), 3.54-3.46 |
| | | | (m, 2H), 3.35-3.31 (m, 1H), 3.23-3.00 (m, |
| | | | 2H), 2.43-2.40 (m, 1H), 2.16-2.07 (m, 1H) |
| 287 | 4.2 | 8.0 | 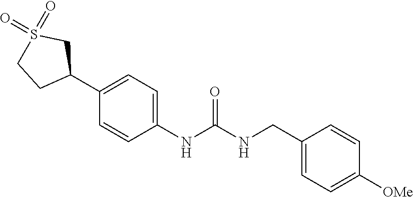 [SMILES: COc1ccc(CNC(=O)Nc2ccc([C@H]3CCS(=O)(=O)C3)cc2)cc1] |
| | | | Compound 287: {[4-((3R)-1,1-dioxothiolan- |
| | | | 3-yl)phenyl]amino}-N-[(4- |
| | | | methoxyphenyl)methyl] carboxamide. |
| | | | LCMS-ESI (POS.) m/z: 375.0 (M + H)+. ¹H |
| | | | NMR (400 MHz, DMSO-d₆) δ 8.52 (s, 1H), |
| | | | 7.40-7.32 (m, 2H), 7.22 (d, J = 8.1 Hz, 4H), |
| | | | 6.94-6.85 (m, 2H), 6.52 (t, J = 5.8 Hz, 1H), |
| | | | 4.21 (d, J = 5.8 Hz, 2H), 3.73 (s, 3H), 3.56- |
| | | | 3.46 (m, 2H), 3.21-3.02 (m, 1H), 3.11-3.00 (m, |
| | | | 1H), 2.44-2.38 (m, 1H), 2.20-2.06 (m, 1H), |
| | | | 1.24 (s, 1H). |
| 294 | 4.2 | 8.0 | 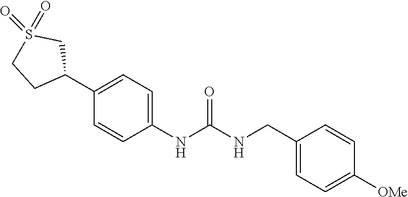 [SMILES: COc1ccc(CNC(=O)Nc2ccc([C@@H]3CCS(=O)(=O)C3)cc2)cc1] |
| | | | Compound 294: {[4-((3S)-1,1-dioxothiolan-3- |
| | | | yl)phenyl]amino}-N-[(4- |
| | | | methoxyphenyl)methyl]carboxamide. |
| | | | LCMS-ESI (POS.) m/z: 375.0 (M + H)+. ¹H |
| | | | NMR (400 MHz, DMSO-d₆) δ 8.52 (s, 1H), |
| | | | 7.36 (d, J = 8.5 Hz, 2H), 7.22 (d, J = 8.0 Hz, |
| | | | 4H), 6.93-6.86 (m, 2H), 6.52 (t, J = 5.9 Hz, |
| | | | 1H), 4.21 (d, J = 5.8 Hz, 2H), 3.73 (s, 3H), 3.50 |
| | | | (dd, J = 8.0, 4.4 Hz, 2H), 3.21-3.00 (m, |
| | | | 2H), 2.44-2.40 (m, 1H), 2.18-1.99 (m, 1H), |
| | | | 1.24 (s, 1H). |

### Example 9

### Synthesis of N-(4-(3-(4-chlorobenzyl)ureido)benzyl)methanesulfonamide (Compound 475)

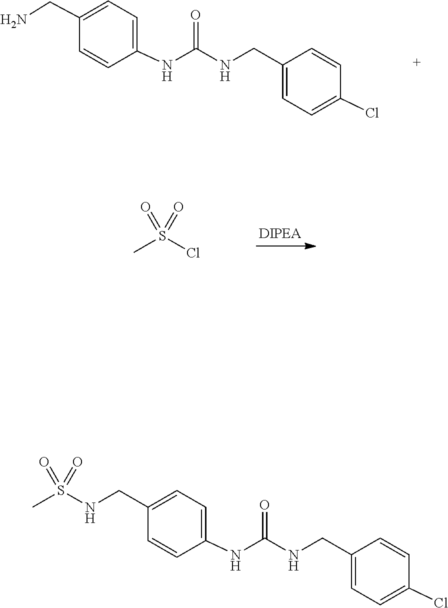

Methanesulfonyl chloride (21 μL, 0.269 mmol, 1.3 equiv) was added to a stirring solution of 1-(4-(aminomethyl)phenyl)-3-(4-chlorobenzyl)urea hydrochloride (60 mg, 0.21 mmol, 1 equiv) and diisopropylethylamine (72 μL, 0.41 mmol, 2 equiv) in DMF (2 mL) at rt. After 1 h, the product was isolated by reverse phase HPLC (5->95% MeCN/H₂O w/0.1% formic acid) as a white solid (20 mg, 26%). LCMS-ESI (POS.) m/z: 368.0 (M+H)+. ¹H NMR (400 MHz, DMSO-d₆) δ 8.60 (s, 1H), 7.44 (t, J=6.2 Hz, 1H), 7.38 (t, J=8.3 Hz, 4H), 7.32 (d, J=8.4 Hz, 2H), 7.19 (d, J=8.5 Hz, 2H), 6.65 (t, J=6.1 Hz, 1H), 4.28 (d, J=5.9 Hz, 2H), 4.05 (d, J=6.3 Hz, 2H), 2.81 (s, 3H).

Compounds in the following table were prepared in a similar manner as Compound 475, using the intermediates and reagents as listed.

| | | Sulfonyl | |
|---|---|---|---|
| Ex # | Amine | Chloride | Structure, Name and Data |
| 475 | Inter- mediate 2.5 | methane- sulfonyl chloride | 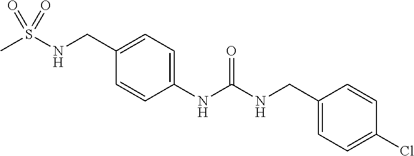 [SMILES: CS(=O)(=O)NCc1ccc(NC(=O)NCc2ccc(Cl)cc2)cc1] |
| | | | N-(4-(3-(4-chlorobenzyl)ureido)benzyl) |
| | | | methanesulfonamide. LCMS-ESI (POS.) m/z: 368.0 |
| | | | (M + H)+. ¹H NMR (400 MHz, DMSO-d₆) δ 8.60 (s, |
| | | | 1H), 7.44 (t, J = 6.2 Hz, 1H), 7.38 (t, J = 8.3 Hz, 4H), |
| | | | 7.32 (d, J = 8.4 Hz, 2H), 7.19 (d, J = 8.5 Hz, 2H), |
| | | | 6.65 (t, J = 6.1 Hz, 1H), 4.28 (d, J = 5.9 Hz, 2H), 4.05 |
| | | | (d, J = 6.3 Hz, 2H), 2.81 (s, 3H). |
| 476 | Inter- mediate 2.5 | pyridine- 3-sulfonyl chloride | 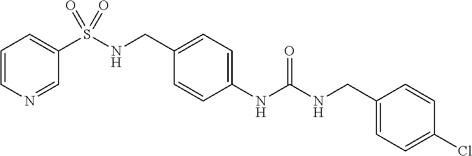 [SMILES: O=C(NCc1ccc(Cl)cc1)Nc1ccc(CNS(=O)(=O)c2cccnc2)cc1] |
| | | | N-(4-(3-(4-chlorobenzyl)ureido)benzyl)pyridine-3- |
| | | | sulfonamide. LCMS-ESI (POS.) m/z: 431.0 |
| | | | (M + H)+. ¹H NMR (400 MHz, DMSO-d₆) δ 8.90 (d, |
| | | | J = 2.2 Hz, 1H), 8.78 (dd, J = 4.9, 1.6 Hz, 1H), 8.56 |
| | | | (s, 1H), 8.31 (t, J = 6.0 Hz, 1H), 8.13 (dt, J = 8.1, 2.0 |
| | | | Hz, 1H), 7.59 (dd, J = 8.0, 4.8 Hz, 1H), 7.39 (d, J = |
| | | | 8.4 Hz, 2H), 7.30 (dd, J = 13.8, 8.5 Hz, 4H), 7.06 (d, |
| | | | J = 8.5 Hz, 2H), 6.63 (t, J = 5.9 Hz, 1H), 4.27 (d, J = |
| | | | 5.9 Hz, 2H), 3.96 (d, J = 6.1 Hz, 2H). |
| 477 | Inter- mediate 2.8 | methane- sulfonyl chloride | 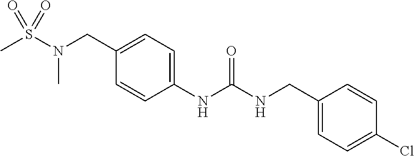 [SMILES: CN(Cc1ccc(NC(=O)NCc2ccc(Cl)cc2)cc1)S(C)(=O)=O] |
| | | | N-(4-(3-(4-chlorobenzyl)ureido)benzyl)-N- |
| | | | methylmethanesulfonamide. LCMS-ESI (POS.) |
| | | | m/z: 382.1 (M + H)+. ¹H NMR (400 MHz, DMSO-d₆) |
| | | | δ 8.65 (s, 1H), 7.40 (dd, J = 8.4, 5.2 Hz, 4H), 7.32 (d, |
| | | | J = 8.5 Hz, 2H), 7.19 (d, J = 8.5 Hz, 2H), 6.66 (t, J = |
| | | | 6.0 Hz, 1H), 4.28 (d, J = 6.0 Hz, 2H), 4.13 (s, 2H), |
| | | | 2.91 (s, 3H), 2.62 (s, 3H). |
| 478 | Inter- mediate 2.8 | tetrahydro- 2H-pyran- 4-sulfonyl chloride | 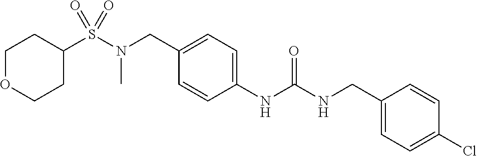 [SMILES: CN(Cc1ccc(NC(=O)NCc2ccc(Cl)cc2)cc1)S(=O)(=O)C1CCOCC1] |
| | | | N-(4-(3-(4-chlorobenzyl)ureido)benzyl)-N- |
| | | | methyltetrahydro-2H-pyran-4-sulfonamide. |
| | | | LCMS-ESI (POS.) m/z: 452.2 (M + H)+. ¹H NMR |
| | | | (400 MHz, DMSO-d₆) δ 8.65 (s, 1H), 7.40 (dd, J = |
| | | | 8.5, 3.3 Hz, 4H), 7.32 (d, J = 8.5 Hz, 2H), 7.18 (d, J = |
| | | | 8.5 Hz, 2H), 6.66 (t, J = 6.0 Hz, 1H), 4.32-4.21 (m, |
| | | | 4H), 3.94 (dd, J = 11.2, 4.0 Hz, 2H), 3.54 (tt, J = |
| | | | 11.8, 3.8 Hz, 1H), 3.35 (td, J = 11.8, 1.8 Hz, 2H), |
| | | | 2.69 (s, 3H), 1.87 (d, J = 12.2 Hz, 2H), 1.68 (qd, J = |
| | | | 12.3, 4.7 Hz, 2H). |
| 500 | Inter- mediate 2.5 | tetrahydro- furan-3- sulfonyl chloride | 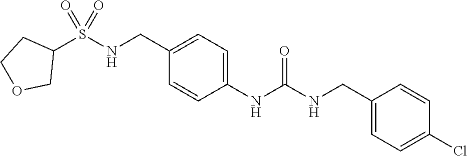 [SMILES: O=C(NCc1ccc(Cl)cc1)Nc1ccc(CNS(=O)(=O)C2CCOC2)cc1] |
| | | | N-(4-(3-(4- |
| | | | chlorobenzyl)ureido)benzyl)tetrahydrofuran-3- |
| | | | sulfonamide. LCMS (POS.) m/z: 424.2 (M + H)+. |
| | | | ¹H NMR (400 MHz, DMSO-d₆) δ: 8.60 (s, 1H), 7.74 |
| | | | (t, J = 6.1 Hz, 1H), 7.44-7.34 (m, 4H), 7.32 (d, J = |
| | | | 8.5 Hz, 2H), 7.22-7.15 (m, 2H), 6.65 (t, J = 6.0 Hz, |
| | | | 1H), 4.28 (d, J = 5.9 Hz, 2H), 4.09 (dd, J = 5.7, 3.9 |
| | | | Hz, 3H), 3.89 (dd, J = 9.5, 5.1 Hz, 1H), 3.85-3.69 |
| | | | (m, 3H), 3.65 (dt, J = 8.3, 6.8 Hz, 1H), 3.17 (d, J = |
| | | | 5.2 Hz, 2H), 2.09 (qd, J = 6.7, 2.9 Hz, 2H). |

### Example 10

### Synthesis of tert-butyl 4-(4-(3-(4-chlorobenzyl)ureido)benzyl)-3-oxopiperazine-1-carboxylate

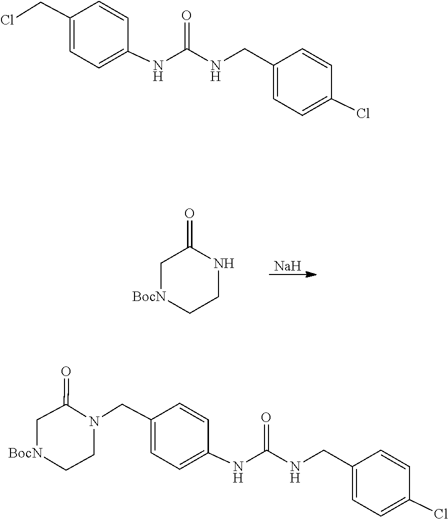

To a stirred solution of tert-butyl 3-oxopiperazine-1-carboxylate (162.00 mg, 0.809 mmol, 1.00 equiv) in DMF (3.00 mL) at 0° C. was added NaH (38.83 mg, 0.971 mmol, 1.20 equiv, 60%). After stirred at 0° C. for 15 min, the resulting mixture at 0° C. was added 1-[4-(chloromethyl)phenyl]-3-[(4-chlorophenyl)methyl]urea (300.18 mg, 0.971 mmol, 1.20 equiv). The resulting mixture was stirred at r.t. for 2 h, quenched by MeOH (2 mL) at 0° C., concentrated under reduced pressure to afford 300 mg of tert-butyl 4-[[4-([[(4-chlorophenyl)methyl]carbamoyl]amino)phenyl]methyl]-3-oxopiperazine-1-carboxylate as a yellow solid. LCMS-ESI (POS.) m/z: 473 (M+H)+.

Compounds in the following table were prepared in a similar manner as tert-butyl 4-(4-(3-(4-chlorobenzyl)ureido)benzyl)-3-oxopiperazine-1-carboxylate, using the intermediates and reagents as listed.

| | Inter- | | |
|---|---|---|---|
| Ex # | mediate | Nucleophile | Structure, Name and Data |
| 369 | Inter- mediate 16 | morpholin-3- one | 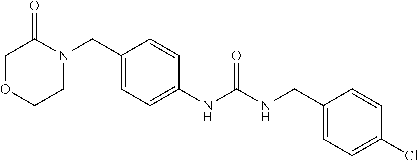 [SMILES: O=C(NCc1ccc(Cl)cc1)Nc1ccc(CN2CCOCC2=O)cc1] |
| | | | 1-(4-chlorobenzyl)-3-(4-((3- |
| | | | oxomorpholino)methyl)phenyl)urea. LCMS-ESI |
| | | | (POS.) m/z: 374 (M + H)+. ¹H NMR (300 MHz, |
| | | | DMSO-d₆) δ: 8.62 (s, 1H), 7.45-7.35 (m, 4H), 7.33 |
| | | | (d, J = 8.2 Hz, 2H), 7.14 (d, J = 8.2 Hz, 2H), 6.64 (d, |
| | | | J = 6.4 Hz, 1H), 4.47 (s, 2H), 4.30 (d, J = 5.8 Hz, |
| | | | 2H), 4.11 (s, 2H), 3.81 (t, J = 5.0 Hz, 2H), 3.24 (d, J = |
| | | | 5.3 Hz, 2H). |
| 378 | Inter- mediate 16 | pyrrolidin-2- one | 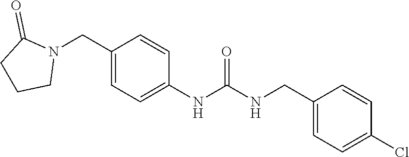 [SMILES: O=C(NCc1ccc(Cl)cc1)Nc1ccc(CN2CCCC2=O)cc1] |
| | | | 1-(4-chlorobenzyl)-3-(4-((2-oxopyrrolidin-1- |
| | | | yl)methyl)phenyl)urea. LCMS-ESI (POS.) m/z: 358 |
| | | | (M + H)+. ¹H NMR (300 MHz, DMSO-d₆) δ: 8.61 (s, |
| | | | 1H), 7.45-7.29 (m, 6H), 7.09 (d, J = 8.5 Hz, 2H), |
| | | | 6.65 (t, J = 6.0 Hz, 1H), 4.29 (d, J = 6.6 Hz, 4H), 3.20 |
| | | | (t, J = 7.0 Hz, 2H), 2.28 (t, J = 8.0 Hz, 2H), 1.91 (p, |
| | | | J = 7.6 Hz, 2H). |
| | Inter- mediate 16 | 3-methyl- pyrrolidin-2- one | 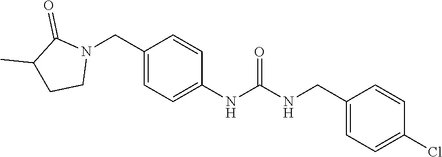 [SMILES: CC1CCN(Cc2ccc(NC(=O)NCc3ccc(Cl)cc3)cc2)C1=O] |
| | | | 1-(4-chlorobenzyl)-3-(4-((3-methyl-2- |
| | | | oxopyrrolidin-1-yl)methyl)phenyl)urea. LCMS- |
| | | | ESI (POS.) m/z: 372 (M + H)+. |
| | Inter- mediate 16 | 5-methyl- pyrrolidin-2- one | 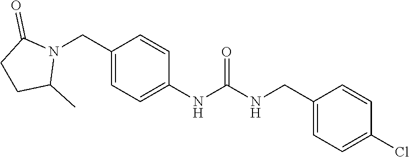 [SMILES: CC1CCC(=O)N1Cc1ccc(NC(=O)NCc2ccc(Cl)cc2)cc1] |
| | | | 1-(4-chlorobenzyl)-3-(4-((2-methyl-5- |
| | | | oxopyrrolidin-1-yl)methyl)phenyl)urea. LCMS- |
| | | | ESI (POS.) m/z: 372 (M + H)+. |
| 391 | Inter- mediate 16 | 4-methyl- piperazin-2- one | 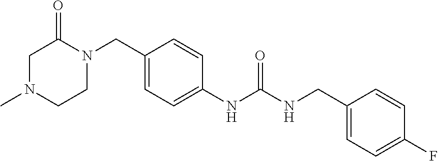 [SMILES: CN1CCN(Cc2ccc(NC(=O)NCc3ccc(F)cc3)cc2)C(=O)C1] |
| | | | 1-(4-fluorobenzyl)-3-(4-((4-methyl-2-oxopiperazin- |
| | | | 1-yl)methyl)phenyl)urea. LCMS-ESI (POS.) m/z: |
| | | | 371 (M + H)+. ¹H NMR (300 MHz, DMSO-d₆) δ: |
| | | | 8.55 (s, 1H), 7.39-7.27 (m, 4H), 7.21-7.03 (m, |
| | | | 4H), 6.59 (t, J = 6.0 Hz, 1H), 4.40 (s, 2H), 4.26 (d, J = |
| | | | 5.8 Hz, 2H), 3.14 (t, J = 5.5 Hz, 2H), 2.97 (s, 2H), |
| | | | 2.53 (d, J = 5.7 Hz, 2H), 2.19 (s, 3H). |

### Example 11

### Synthesis of (S)-1-(4-chlorobenzyl)-3-(4-((3-methyl-2-oxopyrrolidin-1-yl)methyl)phenyl)urea (Compound 379) and (R)-1-(4-chlorobenzyl)-3-(4-((3-methyl-2-oxopyrrolidin-1-yl)methyl)phenyl)urea (Compound 380)

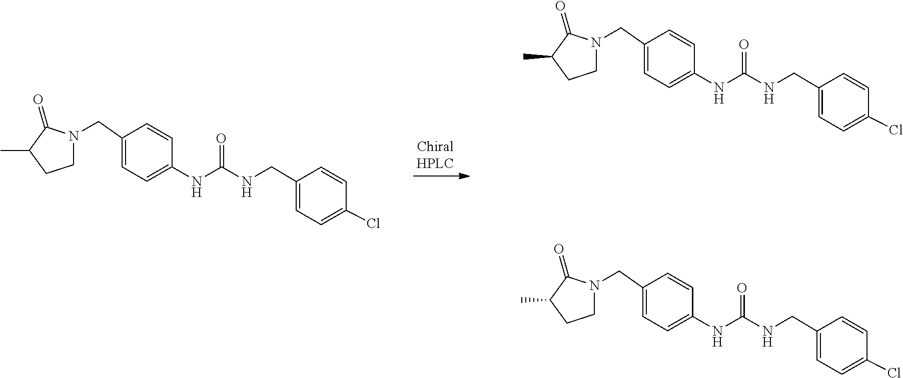 [SMILES: CC1CCN(Cc2ccc(NC(=O)NCc3ccc(Cl)cc3)cc2)C1=O ; C[C@@H]1CCN(Cc2ccc(NC(=O)NCc3ccc(Cl)cc3)cc2)C1=O ; C[C@H]1CCN(Cc2ccc(NC(=O)NCc3ccc(Cl)cc3)cc2)C1=O]

The racemic compound 1-[(4-chlorophenyl) methyl]-3-[4-[(3-methyl-2-oxopyrrolidin-1-yl) methyl]phenyl]urea (70 mg, 0.188 mmol, 1 equiv) was separated by Prep-Chiral-HPLC with the following conditions (Column: CHIRALPAK IF-2, 2*25 cm, 5 um; Mobile Phase A: Hex (8 mmol/L NH3.MeOH)-HPLC, Mobile Phase B: EtOH-HPLC; Flow rate:20 mL/min; Gradient:20 B to 20 B in 35 min; Injection Volume: 0.8 ml; Number Of Runs:6;) to afford 15.6 mg of (S)-1-(4-chlorobenzyl)-3-(4-((3-methyl-2-oxopyrrolidin-1-yl)methyl)phenyl)urea and 19.1 mg of (R)-1-(4-chlorobenzyl)-3-(4-((3-methyl-2-oxopyrrolidin-1-yl)methyl)phenyl)urea as white solids.

N.B. Absolute stereochemistry assigned randomly and not confirmed.

(S)-1-(4-chlorobenzyl)-3-(4-((3-methyl-2-oxopyrrolidin-1-yl)methyl)phenyl)urea. LCMS-ESI (POS.) m/z: 372 (M+H)+. ¹H NMR (300 MHz, DMSO-d₆) δ: 8.57 (s, 1H), 7.43-7.26 (m, 5H), 7.05 (d, J=8.4 Hz, 2H), 6.62 (t, J=6.0 Hz, 1H), 4.27 (d, J=5.3 Hz, 4H), 3.11 (td, J=6.4, 3.0 Hz, 2H), 2.39 (q, J=8.6 Hz, 1H), 1.51 (dt, J=12.5, 8.6 Hz, 1H), 1.23 (s, 2H), 1.07 (d, J=7.1 Hz, 3H).

(R)-1-(4-chlorobenzyl)-3-(4-((3-methyl-2-oxopyrrolidin-1-yl)methyl)phenyl)urea. LCMS-ESI (POS.) m/z: 372 (M+H)+. ¹H NMR (300 MHz, DMSO-d₆) δ: 8.59 (s, 1H), 7.43-7.26 (m, 5H), 7.05 (d, J=8.4 Hz, 2H), 6.64 (t, J=6.0 Hz, 1H), 4.27 (d, J=5.3 Hz, 4H), 3.38 (s, 1H), 3.16-3.06 (m, 2H), 2.39 (q, J=8.3 Hz, 1H), 1.49 (dd, J=12.4, 8.6 Hz, 1H), 1.23 (s, 1H), 1.07 (d, J=7.2 Hz, 3H).

Compounds in the following table were prepared in a similar manner as Compound 379 and Compound 380, using the intermediates and reagents as listed.

| | | Chiral | |
|---|---|---|---|
| Ex # | Racimate | Column | Structure, Name and Data |
| 381 & 382 | 1-(4-chloro- benzyl)-3- (4-((2- methyl-5- oxopyrro- lidin-1- yl)methyl) phenyl)urea | Chiralpak IA | 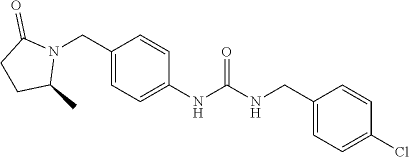 [SMILES: C[C@H]1CCC(=O)N1Cc1ccc(NC(=O)NCc2ccc(Cl)cc2)cc1] |
| | | | 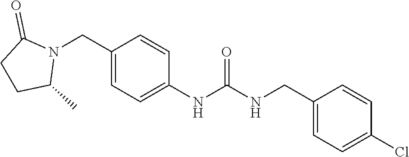 [SMILES: C[C@@H]1CCC(=O)N1Cc1ccc(NC(=O)NCc2ccc(Cl)cc2)cc1] |
| | | | (R)-1-(4-chlorobenzyl)-3-(4-((2-methyl-5- |
| | | | oxopyrrolidin-1-yl)methyl)phenyl)urea. |
| | | | LCMS-ESI (POS.) m/z: 372 (M + H)+. ¹H NMR (300 |
| | | | MHz, DMSO-d₆) δ: 8.59 (s, 1H), 7.45-7.29 (m, |
| | | | 6H), 7.10 (d, J = 8.1 Hz, 2H), 6.63 (d, J = 6.3 Hz, |
| | | | 1H), 4.64 (d, J = 15.0 Hz, 1H), 4.29 (d, J = 6.0 Hz, |
| | | | 2H), 3.97 (d, J = 14.9 Hz, 1H), 3.51-3.41 (m, 1H), |
| | | | 3.32 (s, 2H), 2.51 (s, 2H), 2.38-2.19 (m, 1H), 2.10 |
| | | | (s, 1H), 1.51 (s, 1H), 1.10 (d, J = 6.3 Hz, 3H). |
| | | | (S)-1-(4-chlorobenzyl)-3-(4-((2-methyl-5- |
| | | | oxopyrrolidin-1-yl)methyl)phenyl)urea. |
| | | | LCMS-ESI (POS.) m/z: 372 (M + H)+. ¹H NMR (300 |
| | | | MHz, DMSO-d₆) δ: 8.57 (s, 2H), 7.43-7.23 (m, |
| | | | 12H), 7.08 (d, J = 8.3 Hz, 4H), 6.62 (t, J = 6.0 Hz, |
| | | | 2H), 4.62 (d, J = 15.0 Hz, 2H), 4.27 (d, J = 5.9 Hz, |
| | | | 4H), 3.95 (d, J = 15.0 Hz, 2H), 3.82 (t, J = 7.1 Hz, 1H), |
| | | | 3.38 (s, 1H), 2.36-2.26 (m, 1H), 2.26-2.16 (m, 1H), |
| | | | 2.09 (dt, J = 13.0, 6.4 Hz, 2H), 1.58-1.44 (m, 1H), |
| | | | 1.23 (s, 1H), 1.24-1.12 (m, 2H), 1.08 (d, J = 6.3 Hz, 6H). |
| 370 & 371 | Intermediate 17.1 | Chiralpak ID-2 | 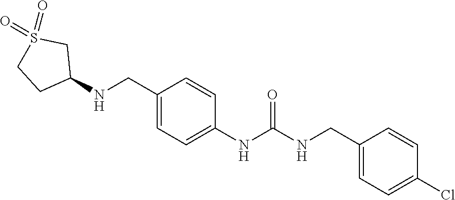 [SMILES: O=C(NCc1ccc(Cl)cc1)Nc1ccc(CN[C@H]2CCS(=O)(=O)C2)cc1] |
| | | | 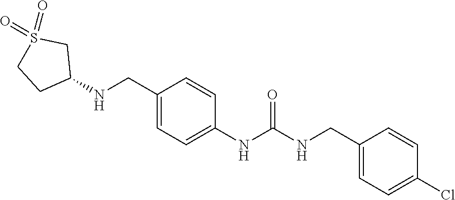 [SMILES: O=C(NCc1ccc(Cl)cc1)Nc1ccc(CN[C@@H]2CCS(=O)(=O)C2)cc1] |
| | | | (R)-1-(4-chlorobenzyl)-3-(4-(((1,1- |
| | | | dioxidotetrahydrothiophen-3- |
| | | | yl)amino)methyl)phenyl)urea. LCMS-ESI (POS.) |
| | | | m/z: 408 (M + H)+. ¹H NMR (300 MHz, DMSO-d₆) |
| | | | δ: 8.52 (s, 1H), 7.43-7.33 (m, 2H), 7.38-7.27 (m, |
| | | | 4H), 7.18 (d, J = 8.5 Hz, 2H), 6.60 (t, J = 6.0 Hz, 1H), |
| | | | 4.27 (d, J = 5.9 Hz, 2H), 3.69-3.53 (m, 2H), 3.39 (q, |
| | | | J = 6.5 Hz, 1H), 3.29-3.14 (m, 3H), 3.09-2.93 (m, |
| | | | 1H), 2.87 (dd, J = 13.1, 6.5 Hz, 1H), 2.27-2.15 (m, |
| | | | 1H), 2.10-1.91 (m, 1H). |
| | | | (S)-1-(4-chlorobenzyl)-3-(4-(((1,1- |
| | | | dioxidotetrahydrothiophen-3- |
| | | | yl)amino)methyl)phenyl)urea. LCMS-ESI (POS.) |
| | | | m/z: 408 (M + H)+. ¹H NMR (300 MHz, DMSO-d₆) |
| | | | δ: 8.52 (s, 1H), 7.43-7.33 (m, 2H), 7.38-7.27 (m, |
| | | | 4H), 7.18 (d, J = 8.5 Hz, 2H), 6.60 (t, J = 6.0 Hz, 1H), |
| | | | 4.27 (d, J = 5.9 Hz, 2H), 3.69-3.53 (m, 2H), 3.39 (q, |
| | | | J = 6.5 Hz, 1H), 3.29-3.14 (m, 3H), 3.09-2.93 (m, |
| | | | 1H), 2.87 (dd, J = 13.1, 6.5 Hz, 1H), 2.27-2.15 (m, |
| | | | 1H), 2.10-1.91 (m, 1H). |
| 372 & 373 | Intermediate 18.1 | Chiralpak ID-2 | 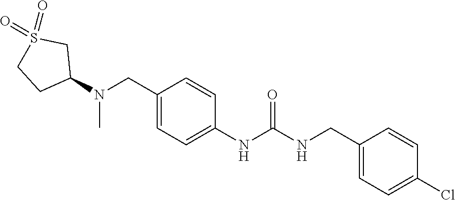 [SMILES: CN(Cc1ccc(NC(=O)NCc2ccc(Cl)cc2)cc1)[C@H]1CCS(=O)(=O)C1] |
| | | | 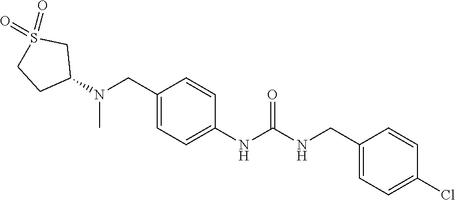 [SMILES: CN(Cc1ccc(NC(=O)NCc2ccc(Cl)cc2)cc1)[C@@H]1CCS(=O)(=O)C1] |
| | | | (S)-1-(4-chlorobenzyl)-3-(4-(((1,1- |
| | | | dioxidotetrahydrothiophen-3- |
| | | | yl)(methyl)amino)methyl)phenyl)urea. LCMS-ESI |
| | | | (POS.) m/z: 422 (M + H)+. ¹H NMR (300 MHz, |
| | | | DMSO-d₆) δ: 8.55 (s, 1H), 7.43-7.27 (m, 6H), 7.14 |
| | | | (d, J = 8.5 Hz, 2H), 6.62 (t, J = 5.7 Hz, 1H), 4.27 (d, J = 5.9 Hz, |
| | | | 2H), 3.44 (q, J = 13.3 Hz, 3H), 3.22 (d, J = 12.4 Hz, |
| | | | 1H), 3.12-3.00 (m, 2H), 2.98 (dd, J = 11.0, 2.8 Hz, 1H), |
| | | | 2.37-2.22 (m, 1H), 2.08 (s, 3H), 2.07-1.98 (m, 1H). |
| | | | (R)-1-(4-chlorobenzyl)-3-(4-(((1,1- |
| | | | dioxidotetrahydrothiophen-3- |
| | | | yl)(methyl)amino)methyl)phenyl)urea. LCMS-ESI |
| | | | (POS.) m/z: 422 (M + H)+. ¹H NMR (300 MHz, |
| | | | DMSO-d₆) δ: 8.55 (s, 1H), 7.43-7.27 (m, 6H), 7.14 |
| | | | (d, J = 8.5 Hz, 2H), 6.62 (t, J = 5.7 Hz, 1H), 4.27 (d, |
| | | | J = 5.9 Hz, 2H), 3.44 (q, J = 13.3 Hz, 3H), 3.22 (d, J = |
| | | | 12.4 Hz, 1H), 3.12-3.00 (m, 2H), 2.98 (dd, J = 11.0, |
| | | | 2.8 Hz, 1H), 2.37-2.22 (m, 1H), 2.08 (s, 3H), 2.07- |
| | | | 1.98 (m, 1H). |
| 374 | Intermediate 17.2 | CHIRAL- PAK IA | 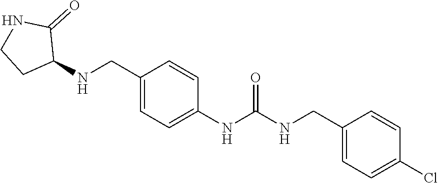 [SMILES: O=C(NCc1ccc(Cl)cc1)Nc1ccc(CN[C@H]2CCNC2=O)cc1] |
| | | | 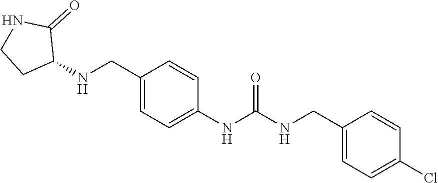 [SMILES: O=C(NCc1ccc(Cl)cc1)Nc1ccc(CN[C@@H]2CCNC2=O)cc1] |
| | | | (S)-1-(4-chlorobenzyl)-3-(4-(((2-oxopyrrolidin-3- |
| | | | yl)amino)methyl)phenyl)urea. LCMS-ESI (POS.) |
| | | | m/z: 373 (M + H)+. ¹H NMR (300 MHz, DMSO-d₆) |
| | | | δ: 8.53 (s, 1H), 7.70 (s, 1H), 7.45-7.32 (m, 4H), |
| | | | 7.33 (dd, J = 4.5, 1.8 Hz, 2H), 7.19 (d, J = 8.4 Hz, |
| | | | 2H), 6.62 (t, J = 6.0 Hz, 1H), 4.29 (d, J = 5.9 Hz, 2H), |
| | | | 3.70 (s, 2H), 3.13 (d, J = 9.4 Hz, 4H), 2.23-2.13 (m, |
| | | | 1H), 1.79-1.63 (m, 1H). |
| | | | (R)-1-(4-chlorobenzyl)-3-(4-(((2-oxopyrrolidin-3- |
| | | | yl)amino)methyl)phenyl)urea. LCMS-ESI (POS.) |
| | | | m/z: 373 (M + H)+. ¹H NMR (300 MHz, DMSO-d₆) |
| | | | δ: 8.53 (s, 1H), 7.70 (s, 1H), 7.45-7.32 (m, 4H), |
| | | | 7.33 (dd, J = 4.5, 1.8 Hz, 2H), 7.19 (d, J = 8.4 Hz, |
| | | | 2H), 6.62 (t, J = 6.0 Hz, 1H), 4.29 (d, J = 5.9 Hz, 2H), |
| | | | 3.70 (s, 2H), 3.13 (d, J = 9.4 Hz, 4H), 2.23-2.13 (m, |
| | | | 1H), 1.79-1.63 (m, 1H). |
| 375 & 376 | Intermediate 17.3 | CHIRAL- PAK IC-3 | 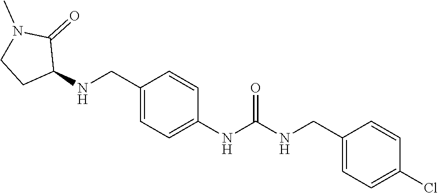 [SMILES: CN1CC[C@H](NCc2ccc(NC(=O)NCc3ccc(Cl)cc3)cc2)C1=O] |
| | | | 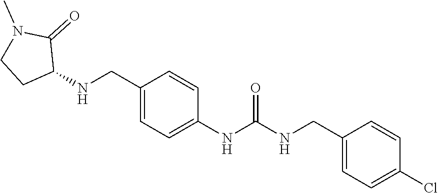 [SMILES: CN1CC[C@@H](NCc2ccc(NC(=O)NCc3ccc(Cl)cc3)cc2)C1=O] |
| | | | (S)-1-(4-chlorobenzyl)-3-(4-(((1-methyl-2- |
| | | | oxopyrrolidin-3-yl)amino)methyl)phenyl)urea. |
| | | | LCMS-ESI (POS.) m/z: 387 (M + H)+. ¹H NMR (300 |
| | | | MHz, DMSO-d₆) δ: 8.51 (s, 1H), 7.43-7.33 (m, 2H), |
| | | | 7.38-7.27 (m, 4H), 7.17 (d, J = 8.4 Hz, 2H), 6.60 (t, |
| | | | J = 6.0 Hz, 1H), 4.27 (d, J = 5.9 Hz, 2H), 3.68 (s, |
| | | | 2H), 3.29-3.11 (m, 4H), 2.71 (s, 3H), 2.23-2.07 |
| | | | (m, 1H), 1.64 (dq, J = 12.4, 8.7 Hz, 1H). |
| | | | (R)-1-(4-chlorobenzyl)-3-(4-(((1-methyl-2- |
| | | | oxopyrrolidin-3-yl)amino)methyl)phenyl)urea. |
| | | | LCMS-ESI (POS.) m/z: 387 (M + H)+. ¹H NMR (300 |
| | | | MHz, DMSO-d₆) δ: 8.51 (s, 1H), 7.43-7.33 (m, 2H), |
| | | | 7.38-7.27 (m, 4H), 7.17 (d, J = 8.4 Hz, 2H), 6.60 (t, |
| | | | J = 6.0 Hz, 1H), 4.27 (d, J = 5.9 Hz, 2H), 3.68 (s, |
| | | | 2H), 3.29-3.11 (m, 4H), 2.71 (s, 3H), 2.23-2.07 |
| | | | (m, 1H), 1.64 (dq, J = 12.4, 8.7 Hz, 1H). |
| 383 | Intermediate 17.4 | CHIRAL- PAK IE | 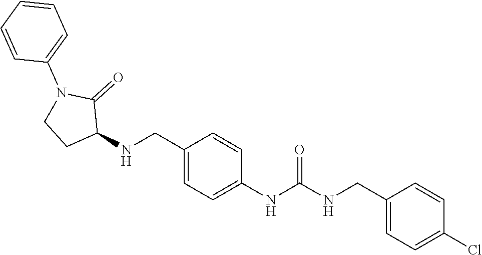 [SMILES: O=C(NCc1ccc(Cl)cc1)Nc1ccc(CN[C@H]2CCN(c3ccccc3)C2=O)cc1] |
| | | | 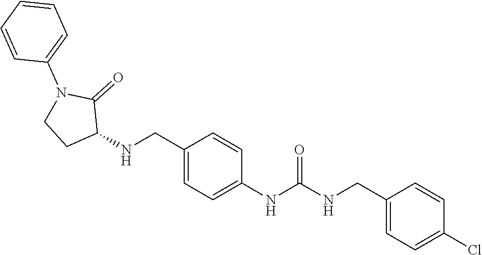 [SMILES: O=C(NCc1ccc(Cl)cc1)Nc1ccc(CN[C@@H]2CCN(c3ccccc3)C2=O)cc1] |
| | | | (S)-1-(4-chlorobenzyl)-3-(4-(((2-oxo-1- |
| | | | phenylpyrrolidin-3-yl)amino)methyl)phenyl)urea. |
| | | | LCMS-ESI (POS.) m/z: 387 (M + H)+. ¹H NMR (300 |
| | | | MHz, DMSO-d₆) δ: 8.53 (s, 1H), 7.71-7.62 (m, 2H), |
| | | | 7.43-7.27 (m, 8H), 7.21 (d, J = 8.3 Hz, 2H), 7.13 (t, |
| | | | J = 7.3 Hz, 1H), 6.61 (s, 1H), 4.27 (d, J = 5.9 Hz, |
| | | | 2H), 3.79-3.66 (m, 3H), 3.46 (t, J = 8.7 Hz, 1H), |
| | | | 2.72 (s, 2H), 2.27 (s, 1H), 1.86-1.72 (m, 1H). |
| | | | (R)-1-(4-chlorobenzyl)-3-(4-(((2-oxo-1- |
| | | | phenylpyrrolidin-3-yl)amino)methyl)phenyl)urea. |
| | | | LCMS-ESI (POS.) m/z: 387 (M + H)+. ¹H NMR (300 |
| | | | MHz, DMSO-d₆) δ: 8.53 (s, 1H), 7.71-7.62 (m, 2H), |
| | | | 7.43-7.27 (m, 8H), 7.21 (d, J = 8.3 Hz, 2H), 7.13 (t, |
| | | | J = 7.3 Hz, 1H), 6.61 (s, 1H), 4.27 (d, J = 5.9 Hz, |
| | | | 2H), 3.79-3.66 (m, 3H), 3.46 (t, J = 8.7 Hz, 1H), |
| | | | 2.72 (s, 2H), 2.27 (s, 1H), 1.86-1.72 (m, 1H). |
| 388 | 1-(4- methoxy- benzyl)- 3-(4-((2- methyl-5- oxopyrro- lidin-1-yl) methyl) phenyl)urea | CHIRAL ART Cellulose- SB | 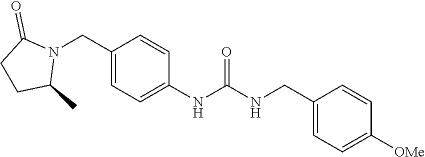 [SMILES: COc1ccc(CNC(=O)Nc2ccc(CN3C(=O)CC[C@@H]3C)cc2)cc1] |
| | | | 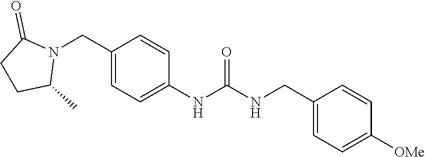 [SMILES: COc1ccc(CNC(=O)Nc2ccc(CN3C(=O)CC[C@H]3C)cc2)cc1] |
| | | | (S)-1-(4-methoxybenzyl)-3-(4-((2-methyl-5- |
| | | | oxopyrrolidin-1-yl)methyl)phenyl)urea. LCMS- |
| | | | ESI (POS.) m/z: 368 (M + H)+. ¹H NMR (300 MHz, |
| | | | DMSO-d₆) δ: 8.48 (s, 1H), 7.34 (d, J = 8.5 Hz, 2H), |
| | | | 7.21 (d, J = 8.5 Hz, 2H), 7.05 (d, J = 8.4 Hz, 2H), |
| | | | 6.88 (d, J = 8.6 Hz, 2H), 6.49 (t, J = 5.9 Hz, 1H), 4.29- |
| | | | 4.16 (m, 4H), 3.72 (s, 3H), 3.11 (dd, J = 9.5, 5.1 Hz, |
| | | | 2H), 2.39 (q, J = 8.0 Hz, 1H), 2.16 (d, J = 13.3 Hz, |
| | | | 1H), 1.59-1.40 (m, 1H), 1.07 (d, J = 7.1 Hz, 3H). |
| | | | (R)-1-(4-methoxybenzyl)-3-(4-((2-methyl-5- |
| | | | oxopyrrolidin-1-yl)methyl)phenyl)urea. LCMS- |
| | | | ESI (POS.) m/z: 368 (M + H)+. ¹H NMR (300 MHz, |
| | | | DMSO-d₆) δ: 8.48 (s, 1H), 7.34 (d, J = 8.5 Hz, 2H), |
| | | | 7.21 (d, J = 8.5 Hz, 2H), 7.05 (d, J = 8.4 Hz, 2H), |
| | | | 6.88 (d, J = 8.6 Hz, 2H), 6.49 (t, J = 5.9 Hz, 1H), 4.29- |
| | | | 4.16 (m, 4H), 3.72 (s, 3H), 3.11 (dd, J = 9.4, 5.1 Hz, |
| | | | 2H), 2.39 (q, J = 8.0 Hz, 1H), 2.16 (d, J = 13.2 Hz, |
| | | | 1H), 1.59-1.40 (m, 1H), 1.07 (d, J = 7.1 Hz, 3H). |
| 389 | 1-(4- methoxy benzyl)- 3-(4-((3- methyl-2- oxopyrro- lidin-1- yl)methyl) phenyl)urea | CHIRAL ART Cellulose- SB | 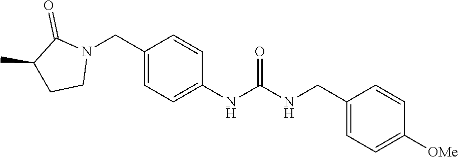 [SMILES: COc1ccc(CNC(=O)Nc2ccc(CN3CC[C@@H](C)C3=O)cc2)cc1] |
| | | | 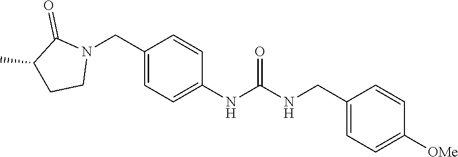 [SMILES: COc1ccc(CNC(=O)Nc2ccc(CN3CC[C@H](C)C3=O)cc2)cc1] |
| | | | (R)-1-(4-methoxybenzyl)-3-(4-((3-methyl-2- |
| | | | oxopyrrolidin-1-yl)methyl)phenyl)urea. LCMS- |
| | | | ESI (POS.) m/z: 368 (M + H)+. ¹H NMR (300 MHz, |
| | | | DMSO-d₆) δ: 8.48 (s, 1H), 7.34 (d, J = 8.5 Hz, 2H), |
| | | | 7.21 (d, J = 8.5 Hz, 2H), 7.05 (d, J = 8.4 Hz, 2H), |
| | | | 6.88 (d, J = 8.6 Hz, 2H), 6.49 (t, J = 5.9 Hz, 1H), 4.29- |
| | | | 4.16 (m, 4H), 3.72 (s, 3H), 3.11 (dd, J = 9.5, 5.1 Hz, |
| | | | 2H), 2.39 (q, J = 8.0 Hz, 1H), 2.16 (d, J = 13.3 Hz, |
| | | | 1H), 1.59-1.40 (m, 1H), 1.07 (d, J = 7.1 Hz, 3H). |
| | | | (S)-1-(4-methoxybenzyl)-3-(4-((3-methyl-2- |
| | | | oxopyrrolidin-1-yl)methyl)phenyl)urea. LCMS- |
| | | | ESI (POS.) m/z: 368 (M + H)+. ¹H NMR (300 MHz, |
| | | | DMSO-d₆) δ: 8.48 (s, 1H), 7.34 (d, J = 8.5 Hz, 2H), |
| | | | 7.21 (d, J = 8.5 Hz, 2H), 7.05 (d, J = 8.4 Hz, 2H), |
| | | | 6.88 (d, J = 8.6 Hz, 2H), 6.49 (t, J = 5.9 Hz, 1H), |
| | | | 4.29-4.16(m, 4H), 3.72 (s, 3H), 3.11 (dd, J = 9.4, 5.1 Hz, |
| | | | 2H), 2.39 (q, J = 8.0 Hz, 1H), 2.16 (d, J = 13.2 Hz, |
| | | | 1H), 1.59-1.40 (m, 1H), 1.07 (d, J = 7.1 Hz, 3H). |
| 365 & 366 | Intermediate 17.5 | Chiralpak ID-2 | 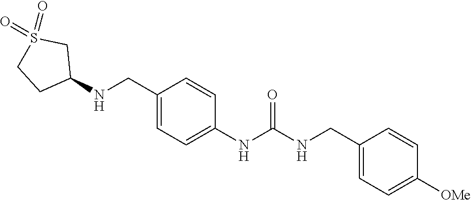 [SMILES: COc1ccc(CNC(=O)Nc2ccc(CN[C@H]3CCS(=O)(=O)C3)cc2)cc1] |
| | | | 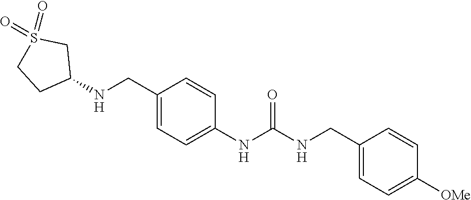 [SMILES: COc1ccc(CNC(=O)Nc2ccc(CN[C@@H]3CCS(=O)(=O)C3)cc2)cc1] |
| | | | (S)-1-(4-(((1,1-dioxidotetrahydrothiophen-3- |
| | | | yl)amino)methyl)phenyl)-3-(4- |
| | | | methoxybenzyl)urea. LCMS-ESI (POS.) m/z: 368 |
| | | | (M + H)+. ¹H NMR (300 MHz, DMSO-d₆) δ: 8.45 (s, 1H), |
| | | | 7.33 (d, J = 8.4 Hz, 2 H), 7.22 (d, J = 8.6 Hz, 2 H), 7.18 (d, |
| | | | J = 8.3 Hz, 2 H), 6.89 (d, J = 8.6 Hz, 2 H), 6.48 (t, J = 5.9 Hz, 1H), |
| | | | 4.21 (d, J = 5.8 Hz, 2 H), 3.73 (s, 3 H), 3.61 (d, J = 5.7 Hz, |
| | | | 2H), 3.40 (t, J = 6.1 Hz, 1 H), 3.33-3.25 (m, 2 H), 3.21 (dd, J = |
| | | | 13.3, 6.8 Hz, 1H), 3.02 (dt, J = 13.4, 7.6 Hz, 1 H), 2.88 (dd, J = |
| | | | 13.1, 6.5 Hz, 1H), 2.23 (dq, J = 12.9, 6.3 Hz, 1 H), 1.99 (dq, J = |
| | | | 15.1, 7.7 Hz, 1H). |
| | | | (R)-1-(4-(((1,1-dioxidotetrahydrothiophen-3- |
| | | | yl)amino)methyl)phenyl)-3-(4- |
| | | | methoxybenzyl)urea. LCMS-ESI (POS.) m/z: 368 |
| | | | (M + H)+. ¹H NMR (300 MHz, DMSO-d₆) δ: 8.47 (s, 1H), |
| | | | 7.34 (d, J = 8.4 Hz, 2 H), 7.22 (d, J = 8.6 Hz, 2 H), 7.14 (d, |
| | | | J = 8.2 Hz, 2 H), 6.89 (d, J = 8.6 Hz, 2 H), 6.49 (t, J = 5.9 Hz, |
| | | | 1H), 4.21 (d, J = 5.8 Hz, 2 H), 3.73 (s, 3 H), 3.53-3.37 (m, 3 H), |
| | | | 3.30-3.20 (m, 2 H), 3.11-2.97 (m, 2 H), 2.35-2.23 (m, 1 H), |
| | | | 2.09 (s, 3 H), 2.07-1.97 (m, 1 H). |
| 367 | Intermediate 18.2 | CHIRAL- PAK IE | 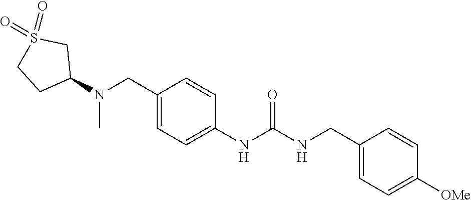 [SMILES: COc1ccc(CNC(=O)Nc2ccc(CN(C)[C@H]3CCS(=O)(=O)C3)cc2)cc1] |
| | | | 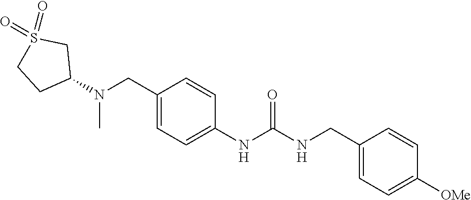 [SMILES: COc1ccc(CNC(=O)Nc2ccc(CN(C)[C@@H]3CCS(=O)(=O)C3)cc2)cc1] |
| | | | (S)-1-(4-(((1,1-dioxidotetrahydrothiophen-3- |
| | | | yl)(methyl)amino)methyl)phenyl)-3-(4- |
| | | | methoxybenzyl)urea. LCMS-ESI (POS.) m/z: 418 |
| | | | (M + H)+. ¹H NMR (300 MHz, DMSO-d₆) δ: 8.47 (s, |
| | | | 1 H), 7.34 (d, J = 8.3 Hz, 2 H), 7.22 (d, J = 8.5 Hz, 2 |
| | | | H), 7.14 (d, J = 8.2 Hz, 2 H), 6.89 (d, J = 8.6 Hz, 2 |
| | | | H), 6.49 (t, J = 6.1 Hz, 1H), 4.21 (d, J = 5.7 Hz, 2 |
| | | | H), 3.73 (s, 3 H), 3.53-3.36 (m, 3 H), 3.29-3.19 (m, 2 |
| | | | H), 3.11-2.95 (m, 2 H), 2.36-2.23 (m, 1 H), 2.09 (s, 3 |
| | | | H), 2.08-1.98 (m, 1 H). |
| | | | (R)-1-(4-(((1,1-dioxidotetrahydrothiophen-3- |
| | | | yl)(methyl)amino)methyl)phenyl)-3-(4- |
| | | | methoxybenzyl)urea. LCMS-ESI (POS.) m/z: 418 |
| | | | (M + H)+. ¹H NMR (300 MHz, DMSO-d₆) δ: 8.44 (s, |
| | | | 1 H), 7.33 (d, J = 8.4 Hz, 2 H), 7.22 (d, J = 8.6 Hz, 2 |
| | | | H), 7.18 (d, J = 8.3 Hz, 2 H), 6.89 (d, J = 8.6 Hz, 2 |
| | | | H), 6.48 (t, J = 5.9 Hz, 1 H), 4.21 (d, J = 5.8 Hz, 2 |
| | | | H), 3.73 (s, 3 H), 3.70 (s, 2 H), 3.29-3.15 (m, 3 H), |
| | | | 2.72 (s, 3 H), 2.22-2.10 (m, 1 H), 1.71-1.58 (m, 1 H). |
| 360 | Intermediate 17.6 | CHIRAL- PAK IG | 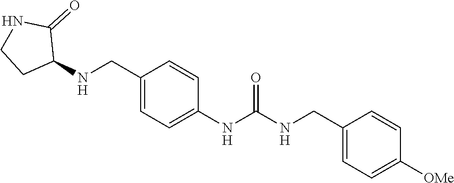 [SMILES: COc1ccc(CNC(=O)Nc2ccc(CN[C@H]3CCNC3=O)cc2)cc1] |
| | | | 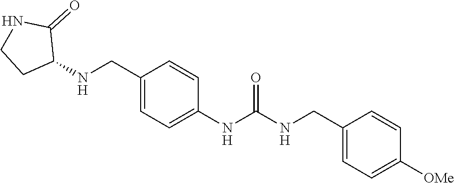 [SMILES: COc1ccc(CNC(=O)Nc2ccc(CN[C@@H]3CCNC3=O)cc2)cc1] |
| | | | (S)-1-(4-methoxybenzyl)-3-(4-(((2-oxopyrrolidin-3- |
| | | | yl)amino)methyl)phenyl)urea. LCMS-ESI (POS.) |
| | | | m/z: 369 (M + H)+. ¹H NMR (300 MHz, DMSO-d₆) |
| | | | δ: 8.44 (s, 1 H), 7.71 (s, 1 H), 7.33 (d, J = 8.4 Hz, 2 |
| | | | H), 7.22 (d, J = 8.6 Hz, 2 H), 7.18 (d, J = 8.3 Hz, 2 |
| | | | H), 6.89 (d, J = 8.6 Hz, 2 H), 6.48 (t, J = 5.9 Hz, 1 |
| | | | H), 4.21 (d, J = 5.8 Hz, 2 H), 3.73 (s, 3 H), 3.68 (d, |
| | | | J = 4.0 Hz, 2 H), 3.32-3.27 (m, 1 H), 3.20-3.03 (m, 3 |
| | | | H), 2.19 (dt, J = 13.6, 7.4 Hz, 1 H), 1.74-1.61 (m, 1 H). |
| | | | (R)-1-(4-methoxybenzyl)-3-(4-(((2-oxopyrrolidin- |
| | | | 3-yl)amino)methyl)phenyl)urea. LCMS-ESI |
| | | | (POS.) m/z: 369 (M + H)+. ¹H NMR (300 MHz, |
| | | | DMSO-d₆) δ: 8.45 (s, 1 H), 7.70 (s, 1 H), 7.33 (d, J = |
| | | | 8.5 Hz, 2 H), 7.22 (d, J = 8.5 Hz, 2 H), 7.17 (d, J = |
| | | | 8.3 Hz, 2 H), 6.89 (d, J = 8.6 Hz, 2 H), 6.49 (t, J = 5.8 |
| | | | Hz, 1 H), 4.21 (d, J = 5.8 Hz, 2 H), 3.73 (s, 3 H), 3.68 |
| | | | (d, J = 4.3 Hz, 2 H), 3.32-3.27 (m, 1 H), 3.20-3.04 |
| | | | (m, 3 H), 2.19 (dt, J = 11.9, 5.8 Hz, 1 H), 1.75-1.59 |
| | | | (m, 1 H). |
| 396 & 549 | 1-(4-chloro- benzyl)-3- (4-(4- methyl- 5-oxo- piperazin- 2-yl)phenyl) urea | CHIRAL ART Cellulose- SB | 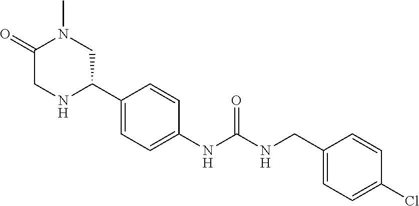 [SMILES: CN1C[C@H](c2ccc(NC(=O)NCc3ccc(Cl)cc3)cc2)NCC1=O] |
| | | | 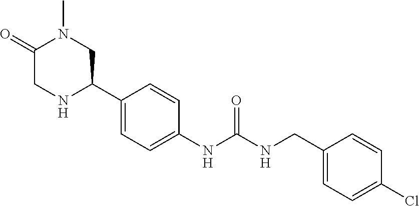 [SMILES: CN1C[C@@H](c2ccc(NC(=O)NCc3ccc(Cl)cc3)cc2)NCC1=O] |
| | | | (S)-1-(4-chlorobenzyl)-3-(4-(4-methyl-5- |
| | | | oxopiperazin-2-yl)phenyl)urea. LCMS-ESI (POS.) |
| | | | m/z: 371 (M + H)+. ¹H NMR (400 MHz, DMSO-d₆) δ |
| | | | 8.61 (s, 1H), 7.38 (t, J = 8.7 Hz, 4H), 7.32 (d, J = 8.5 |
| | | | Hz, 2H), 7.26 (d, J = 8.5 Hz, 2H), 6.66 (t, J = 6.0 Hz, |
| | | | 1H), 4.28 (d, J = 6.0 Hz, 2H), 3.94 (dd, J = 10.4, 4.1 |
| | | | Hz, 1H), 3.42-3.31 (m, 4H), 3.22 (t, J = 11.0 Hz, |
| | | | 1H), 2.83 (s, 3H). |
| | | | (R)-1-(4-chlorobenzyl)-3-(4-(4-methyl-5- |
| | | | oxopiperazin-2-yl)phenyl)urea. LCMS-ESI (POS.) |
| | | | m/z: 371 (M + H)+. ¹H NMR (400 MHz, DMSO-d₆) δ |
| | | | 8.60 (s, 1H), 7.38 (t, J = 8.8 Hz, 4H), 7.32 (d, J = 8.5 |
| | | | Hz, 2H), 7.26 (d, J = 8.5 Hz, 2H), 6.66 (t, J = 6.0 Hz, |
| | | | 1H), 4.28 (d, J = 6.0 Hz, 2H), 3.94 (dd, J = 10.4, 4.1 |
| | | | Hz, 1H), 3.43-3.32 (m, 4H), 3.22 (t, J = 11.0 Hz, |
| | | | 1H), 2.83 (s, 3H). |
| 392 & 393 | 1-(4-chloro- benzyl)-3- (4-(1- methyl-5- oxopipe- razin-2- yl)phenyl) ure | CHIRAL- PAK IG | 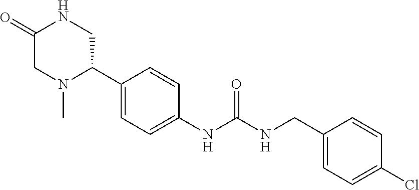 [SMILES: CN1CC(=O)NC[C@@H]1c1ccc(NC(=O)NCc2ccc(Cl)cc2)cc1] |
| | | | 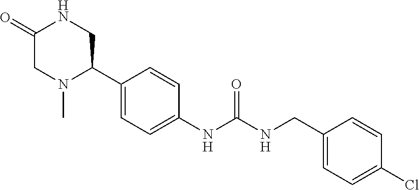 [SMILES: CN1CC(=O)NC[C@H]1c1ccc(NC(=O)NCc2ccc(Cl)cc2)cc1] |
| | | | (S)-1-(4-chlorobenzyl)-3-(4-(1-methyl-5- |
| | | | oxopiperazin-2-yl)phenyl)urea. LCMS-ESI (POS.) |
| | | | m/z: 371 (M + H). ¹H NMR (400 MHz, DMSO-d₆) δ |
| | | | 8.63 (s, 1H), 7.86 (d, J = 3.5 Hz, 1H), 7.39 (dd, J = |
| | | | 8.6, 2.2 Hz, 4H), 7.32 (d, J = 8.5 Hz, 2H), 7.20 (d, J = |
| | | | 8.6 Hz, 2H), 6.66 (t, J = 6.0 Hz, 1H), 4.28 (d, J = 6.0 |
| | | | Hz, 2H), 3.38-3.28 (m, 3H), 3.10 (td, J = 8.3, 7.5, |
| | | | 3.4 Hz, 1H), 2.80 (d, J = 16.6 Hz, 1H), 1.89 (s, 3H). |
| | | | (R)-1-(4-chlorobenzyl)-3-(4-(1-methyl-5- |
| | | | oxopiperazin-2-yl)phenyl)urea. LCMS-ESI (POS.) |
| | | | m/z: 371 (M + H)+. ¹H NMR (400 MHz, DMSO-d₆) δ |
| | | | 8.65 (s, 1H), 7.85 (d, J = 3.5 Hz, 1H), 7.39 (dd, J = |
| | | | 8.5, 2.2 Hz, 4H), 7.32 (d, J = 8.4 Hz, 2H), 7.20 (d, J = |
| | | | 8.3 Hz, 2H), 6.67 (t, J = 5.9 Hz, 1H), 4.28 (d, J = 5.9 |
| | | | Hz, 2H), 3.40-3.27 (m, 3H), 3.10 (td, J = 8.3, 7.5, |
| | | | 3.4 Hz, 1H), 2.80 (d, J = 16.7 Hz, 1H), 1.89 (s, 3H). |
| 397 & 550 | 1-(4-chloro- benzyl)-3- (4-(1,4- dimethyl-5- oxopipe- razin-2- yl)phenyl) urea | CHIRAL- PAK IE | 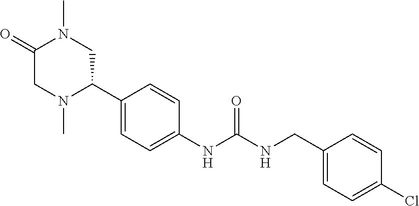 [SMILES: CN1C[C@H](c2ccc(NC(=O)NCc3ccc(Cl)cc3)cc2)N(C)CC1=O] |
| | | | 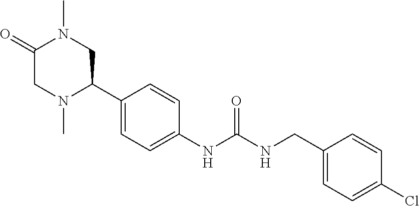 [SMILES: CN1C[C@@H](c2ccc(NC(=O)NCc3ccc(Cl)cc3)cc2)N(C)CC1=O] |
| | | | (S)-1-(4-chlorobenzyl)-3-(4-(1,4-dimethyl-5- |
| | | | oxopiperazin-2-yl)phenyl)urea. LCMS-ESI (POS.) |
| | | | m/z: 387 (M + H)+. ¹H NMR (400 MHz, DMSO-d₆) δ |
| | | | 8.66 (s, 1H), 7.40 (dd, J = 8.5, 3.8 Hz, 4H), 7.32 (d, |
| | | | J = 8.5 Hz, 2H), 7.21 (d, J = 8.5 Hz, 2H), 6.68 (t, J = |
| | | | 6.0 Hz, 1H), 4.28 (d, J = 5.9 Hz, 2H), 3.46-3.34 (m, |
| | | | 2H), 3.33-3.13 (m, 2H), 2.82 (s, 4H), 1.89 (s, 3H). |
| | | | (R)-1-(4-chlorobenzyl)-3-(4-(1,4-dimethyl-5- |
| | | | oxopiperazin-2-yl)phenyl)urea. LCMS-ESI (POS.) |
| | | | m/z: 387 (M + H)+. ¹H NMR (400 MHz, DMSO-d₆) δ |
| | | | 8.66 (s, 1H), 7.40 (dd, J = 8.5, 3.8 Hz, 4H), 7.32 (d, |
| | | | J = 8.3 Hz, 2H), 7.21 (d, J = 8.2 Hz, 2H), 6.68 (t, J = |
| | | | 6.0 Hz, 1H), 4.28 (d, J = 5.9 Hz, 2H), 3.44-3.34 (m, |
| | | | 2H), 3.32-3.11 (m, 2H), 2.84 (d, J = 17.3 Hz, 4H), |
| | | | 1.89 (s, 3H). |
| 400 & 551 | 1-(4-chloro- benzyl)-3- (4-(1,4- dimethyl-6- oxopipe- razin-2-yl) phenyl)urea. | CHIRAL- PAK AD-H | 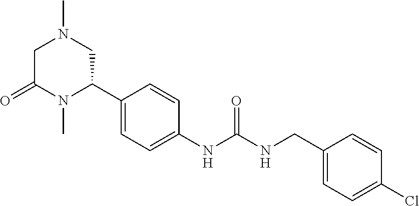 [SMILES: CN1CC(=O)N(C)[C@@H](c2ccc(NC(=O)NCc3ccc(Cl)cc3)cc2)C1] |
| | | | 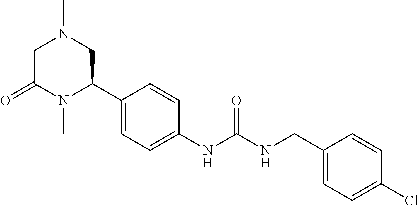 [SMILES: CN1CC(=O)N(C)[C@H](c2ccc(NC(=O)NCc3ccc(Cl)cc3)cc2)C1] |
| | | | (S)-1-(4-chlorobenzyl)-3-(4-(1,4-dimethyl-6- |
| | | | oxopiperazin-2-yl)phenyl)urea. LCMS-ESI (POS.) |
| | | | m/z: 387 (M + H)+. ¹H NMR (300 MHz, DMSO-d₆) |
| | | | δ: 8.62 (s, 1H), 7.43-7.34 (m, 4H), 7.31 (d, J = 8.5 |
| | | | Hz, 2H), 7.12 (d, J = 8.3 Hz, 2H), 6.64 (t, J = 6.0 Hz, |
| | | | 1H), 4.43 (t, J = 5.2 Hz, 1H), 4.27 (d, J = 5.9 Hz, 2H), |
| | | | 3.12 (d, J = 16.1 Hz, 1H), 2.94 (d, J = 16.0 Hz, 1H), |
| | | | 2.79 (dd, J = 12.0, 4.6 Hz, 1H), 2.58 (s, 3H), 2.48 (s, |
| | | | 1H), 2.14 (s, 3H). |
| | | | (R)-1-(4-chlorobenzyl)-3-(4-(1,4-dimethyl-6- |
| | | | oxopiperazin-2-yl)phenyl)urea. LCMS-ESI (POS.) |
| | | | m/z: 387 (M + H)+. ¹H NMR (300 MHz, DMSO-d₆) |
| | | | δ: 8.62 (s, 1H), 7.43-7.33 (m, 4H), 7.36-7.23 (m, |
| | | | 2H), 7.12 (d, J = 8.5 Hz, 2H), 6.64 (t, J = 6.1 Hz, 1H), |
| | | | 4.43 (t, J = 5.2 Hz, 1H), 4.27 (d, J = 5.9 Hz, 2H), 3.13 |
| | | | (d, J = 16.1 Hz, 1H), 2.94 (d, J = 16.1 Hz, 1H), 2.80 |
| | | | (dd, J = 12.0, 4.5 Hz, 1H), 2.58 (s, 3H), 2.48 (s, 1H), |
| | | | 2.14 (s, 3H). |
| 399 & 552 | 1-(4- methoxy- benzyl)- 3-(4-(1- methyl- 5-oxopipe- razin-2-yl) phenyl)urea. | CHIRAL ART Cellulose- SB | 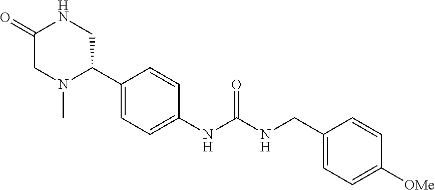 [SMILES: COc1ccc(CNC(=O)Nc2ccc([C@H]3CNC(=O)CN3C)cc2)cc1] |
| | | | 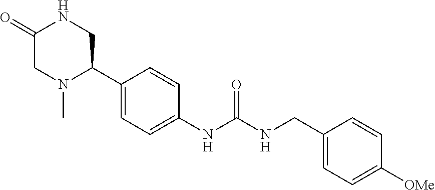 [SMILES: COc1ccc(CNC(=O)Nc2ccc([C@@H]3CNC(=O)CN3C)cc2)cc1] |
| | | | (S)-1-(4-methoxybenzyl)-3-(4-(1-methyl-5- |
| | | | oxopiperazin-2-yl)phenyl)urea. LCMS-ESI (POS.) |
| | | | m/z: 369 (M + H)+. ¹H NMR (300 MHz, DMSO-d₆) |
| | | | δ: 8.52 (s, 1H), 7.84 (d, J = 3.5 Hz, 1H), 7.37 (d, J = |
| | | | 8.5 Hz, 2H), 7.27-7.14 (m, 4H), 6.94-6.84 (m, |
| | | | 2H), 6.51 (t, J = 5.9 Hz, 1H), 4.20 (d, J = 5.8 Hz, 2H), |
| | | | 3.72 (s, 3H), 3.33 (d, J = 16.7 Hz, 1H), 3.21 (s, 2H), |
| | | | 3.08 (s, 1H), 2.79 (d, J = 16.7 Hz, 1H), 1.88 (s, 3H). |
| | | | (R)-1-(4-methoxybenzyl)-3-(4-(1-methyl-5- |
| | | | oxopiperazin-2-yl)phenyl)urea. LCMS-ESI (POS.) |
| | | | m/z: 369 (M + H)+. ¹H NMR (300 MHz, DMSO-d₆) |
| | | | δ: 8.52 (s, 1H), 7.84 (s, 1H), 7.37 (d, J = 8.3 Hz, 2H), |
| | | | 7.21 (dt, J = 7.9, 4.0 Hz, 4H), 6.94-6.84 (m, 2H), |
| | | | 6.51 (s, 1H), 4.20 (d, J = 5.7 Hz, 2H), 3.72 (s, 3H), |
| | | | 3.21 (s, 2H), 3.08 (s, 1H), 2.79 (d, J = 16.7 Hz, 1H), |
| | | | 1.88 (s, 3H). |
| 398 | 1-(4-fluoro- benzyl)-3- (4-(1- methyl- 5-oxopipe- razin-2-yl) phenyl)urea. | CHIRAL ART Cellulose- SB | 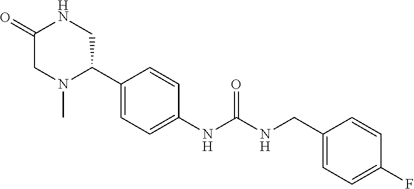 [SMILES: CN1CC(=O)NC[C@@H]1c1ccc(NC(=O)NCc2ccc(F)cc2)cc1] |
| | | | 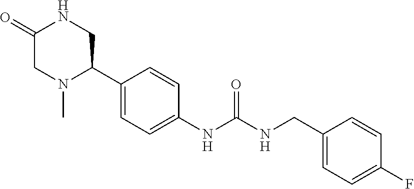 [SMILES: CN1CC(=O)NC[C@H]1c1ccc(NC(=O)NCc2ccc(F)cc2)cc1] |
| | | | (S)-1-(4-fluorobenzyl)-3-(4-(1-methyl-5- |
| | | | oxopiperazin-2-yl)phenyl)urea. LCMS-ESI (POS.) |
| | | | m/z: 357 (M + H)+. ¹H NMR (300 MHz, DMSO-d₆) |
| | | | δ: 8.63 (s, 1H), 7.84 (d, J = 3.5 Hz, 1H), 7.42-7.27 |
| | | | (m, 4H), 7.19 (d, J = 8.4 Hz, 2H), 7.20-7.08 (m, |
| | | | 2H), 6.66 (q, J = 5.1 Hz, 1H), 4.26 (d, J = 5.9 Hz, |
| | | | 2H), 3.32 (d, J = 16.6 Hz, 1H), 3.20 (d, J = 5.1 Hz, |
| | | | 2H), 3.08 (s, 1H), 2.79 (d, J = 16.6 Hz, 1H), 1.88 (s, 3H). |
| | | | (R)-1-(4-fluorobenzyl)-3-(4-(1-methyl-5- |
| | | | oxopiperazin-2-yl)phenyl)urea. LCMS-ESI (POS.) |
| | | | m/z: 357 (M + H)+. ¹H NMR (300 MHz, DMSO-d₆) |
| | | | δ: 8.60 (s, 1H), 7.84 (d, J = 3.5 Hz, 1H), 7.42-7.27 |
| | | | (m, 4H), 7.24-7.15 (m, 2H), 7.20-7.08 (m, 2H), |
| | | | 6.63 (s, 1H), 4.26 (d, J = 5.9 Hz, 2H), 3.32 (d, J = |
| | | | 16.7 Hz, 1H), 3.20 (d, J = 5.2 Hz, 2H), 3.08 (s, 1H), |
| | | | 2.79 (d, J = 16.6 Hz, 1H), 1.88 (s, 3H). |
| 422 & 423 | 1-(4-chloro- benzyl)-3- (4-((4,5- dimethyl- 2-oxopipe- razin-1- yl)methyl) phenyl)urea | CHIRAL ART Cellulose- SB | 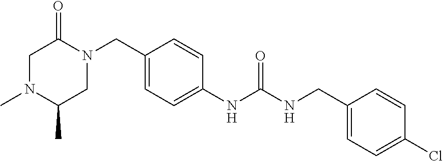 [SMILES: C[C@@H]1CN(Cc2ccc(NC(=O)NCc3ccc(Cl)cc3)cc2)C(=O)CN1C] |
| | | | 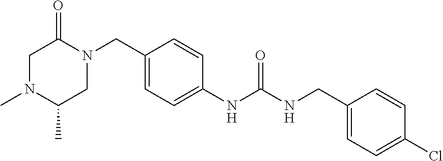 [SMILES: C[C@H]1CN(Cc2ccc(NC(=O)NCc3ccc(Cl)cc3)cc2)C(=O)CN1C] |
| | | | (R)-1-(4-chlorobenzyl)-3-(4-((4,5-dimethyl-2- |
| | | | oxopiperazin-1-yl)methyl)phenyl)urea. LCMS-ESI |
| | | | (POS.) m/z: 401 (M + H)+. ¹H NMR (300 MHz, |
| | | | DMSO-d₆) δ: 8.60 (s, 1H), 7.43-7.32 (m, 4H), 7.36- |
| | | | 7.28 (m, 2H), 7.13-7.06 (m, 2H), 6.64 (t, J = 6.0 |
| | | | Hz, 1H), 4.47 (d, J = 14.4 Hz, 1H), 4.33 (d, J = 14.5 |
| | | | Hz, 1H), 4.28 (d, J = 6.0 Hz, 2H), 3.28 (d, J = 16.6 |
| | | | Hz, 1H), 3.08 (dd, J = 11.7, 4.0 Hz, 1H), 2.91-2.81 |
| | | | (m, 2H), 2.45 (ddd, J = 10.0, 6.4, 3.9 Hz, 1H), 2.14 (s, |
| | | | 3H), 0.97 (d, J = 6.3 Hz, 3H). |
| | | | (S)-1-(4-chlorobenzyl)-3-(4-((4,5-dimethyl-2- |
| | | | oxopiperazin-1-yl)methyl)phenyl)urea. LCMS-ESI |
| | | | (POS.) m/z: 401 (M + H)+. ¹H NMR (300 MHz, |
| | | | DMSO-d₆) δ: 8.60 (s, 1H), 7.43-7.28 (m, 6H), 7.13-7.05 |
| | | | (m, 2H), 6.64 (t, J = 6.1 Hz, 1H), 4.47 (d, J = 14.5 Hz, |
| | | | 1H), 4.33 (d, J = 14.5 Hz, 1H), 4.28 (d, J = 6.0 Hz, |
| | | | 2H), 3.28 (d, J = 16.6 Hz, 1H), 3.08 (dd, J = 11.8, |
| | | | 4.0 Hz, 1H), 2.91-2.81 (m, 2H), 2.44 (ddd, J = 10.0, |
| | | | 6.4, 3.9 Hz, 1H), 2.14 (s, 3H), 0.97 (d, J = 6.3 Hz, 3H). |
| 415 & 416 | 1-(4-chloro- benzyl)-3- (4-((2,4- dimethyl-6- oxopipe- razin-1- yl)methyl) phenyl)urea. | CHIRAL ART Cellulose- SB | 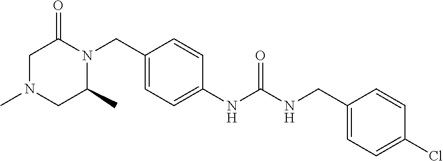 [SMILES: C[C@H]1CN(C)CC(=O)N1Cc1ccc(NC(=O)NCc2ccc(Cl)cc2)cc1] |
| | | | 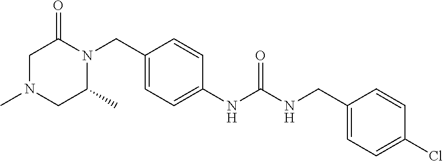 [SMILES: C[C@@H]1CN(C)CC(=O)N1Cc1ccc(NC(=O)NCc2ccc(Cl)cc2)cc1] |
| | | | (S)-1-(4-chlorobenzyl)-3-(4-((2,4-dimethyl-6- |
| | | | oxopiperazin-1-yl)methyl)phenyl)urea. LCMS- |
| | | | APCI (POS.) m/z: 401 (M + H)⁺. ¹H NMR (300 MHz, |
| | | | DMSO-d₆) δ: 8.58 (s, 1H), 7.52-7.22 (m, 6H), 7.09 |
| | | | (d, J = 8.5 Hz, 2H), 6.63 (t, J = 6.0 Hz, 1H), 4.95 (d, |
| | | | J = 15.0 Hz, 1H), 4.28 (d, J = 5.9 Hz, 2H), 3.99 (d, J = |
| | | | 15.0 Hz, 1H), 3.30 (d, J = 0.9 Hz, 1H), 3.13 (d, J = |
| | | | 16.3 Hz, 1H), 2.85 (d, J = 16.2 Hz, 1H), 2.46 (t, J = |
| | | | 4.3 Hz, 2H), 2.19 (s, 3H), 1.17 (d, J = 6.3 Hz, 3H) |
| | | | (R)-1-(4-chlorobenzyl)-3-(4-((2,4-dimethyl-6- |
| | | | oxopiperazin-1-yl)methyl)phenyl)urea. LCMS- |
| | | | APCI (POS.) m/z: 401 (M + H) ¹H NMR (300 MHz, |
| | | | DMSO-d₆) δ: 8.58 (s, 1H), 7.52-7.22 (m, 6H), 7.09 |
| | | | (d, J = 8.5 Hz, 2H), 6.63 (t, J = 6.0 Hz, 1H), 4.95 (d, |
| | | | J = 15.0 Hz, 1H), 4.28 (d, J = 5.9 Hz, 2H), 3.99 (d, J = |
| | | | 15.0 Hz, 1H), 3.30 (d, J = 0.9 Hz, 1H), 3.13 (d, J = |
| | | | 16.3 Hz, 1H), 2.85 (d, J = 16.2 Hz, 1H), 2.46 (t, J = |
| | | | 4.3 Hz, 2H), 2.19 (s, 3H), 1.17 (d, J = 6.3 Hz, 3H). |
| 424 | 1-(4-((4,5- dimethyl-2- oxopipe- razin-1- yl)methyl) phenyl)-3- (4-methoxy- benzyl)urea | CHIRAL ART Cellulose- SB | 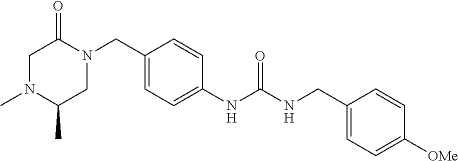 [SMILES: COc1ccc(CNC(=O)Nc2ccc(CN3C[C@@H](C)N(C)CC3=O)cc2)cc1] |
| | | | 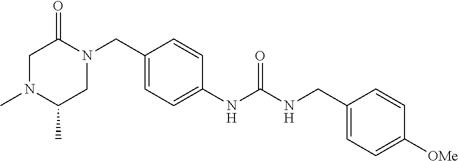 [SMILES: COc1ccc(CNC(=O)Nc2ccc(CN3C[C@H](C)N(C)CC3=O)cc2)cc1] |
| | | | (R)-1-(4-((4,5-dimethyl-2-oxopiperazin-1- |
| | | | yl)methyl)phenyl)-3-(4-methoxybenzyl)urea. |
| | | | LCMS-APCI (POS.) m/z: 397 (M + H)⁺. ¹H NMR |
| | | | (300 MHz, DMSO-d₆) δ: 8.50 (s, 1H), 7.39-7.31 (m, |
| | | | 2H), 7.26-7.18 (m, 2H), 7.12-7.06 (m, 2H), 6.93- |
| | | | 6.85 (m, 2H), 6.50 (t, J = 5.9 Hz, 1H), 4.46 (d, J = |
| | | | 14.5 Hz, 1H), 4.33 (d, J = 14.4 Hz, 1H), 4.21 (d, J = |
| | | | 5.8 Hz, 2H), 3.73 (s, 3H), 3.28 (d, J = 16.6 Hz, 1H), |
| | | | 3.08 (dd, J = 11.8, 3.9 Hz, 1H), 2.91-2.81 (m, 2H), |
| | | | 2.45 (ddd, J = 10.0, 6.5, 4.0 Hz, 1H), 2.14 (s, 3H), |
| | | | 0.97 (d, J = 6.2 Hz, 3H). |
| | | | (S)-1-(4-((4,5-dimethyl-2-oxopiperazin-1- |
| | | | yl)methyl)phenyl)-3-(4-methoxybenzyl)urea. |
| | | | LCMS-APCI (POS.) m/z: 397 (M + H) ⁴H NMR |
| | | | (300 MHz, DMSO-d₆) δ: 8.50 (s, 1H), 7.39-7.31 (m, |
| | | | 2H), 7.26-7.18 (m, 2H), 7.13-7.05 (m, 2H), 6.93- |
| | | | 6.85 (m, 2H), 6.50 (t, J = 5.9 Hz, 1H), 4.46 (d, J = |
| | | | 14.5 Hz, 1H), 4.33 (d, J = 14.5 Hz, 1H), 4.21 (d, J = |
| | | | 5.8 Hz, 2H), 3.73 (s, 3H), 3.08 (dd, J = 11.7, 4.0 Hz, |
| | | | 1H), 2.91-2.81 (m, 2H), 2.45 (ddd, J = 10.0, 6.4, 3.9 |
| | | | Hz, 1H), 2.14 (s, 3H), 0.97 (d, J = 6.2 Hz, 3H). |
| 425 & 426 | 1-(4-((2,4- dimethyl- 6-oxopipe- razin-1-yl) methyl) phenyl)-3- (4-methoxy- benzyl)urea. | CHIRAL ART Cellulose- SB | 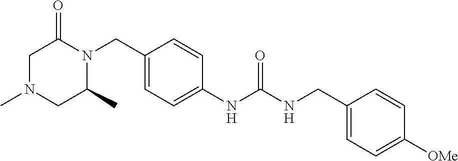 [SMILES: COc1ccc(CNC(=O)Nc2ccc(CN3C(=O)CN(C)C[C@@H]3C)cc2)cc1] |
| | | | 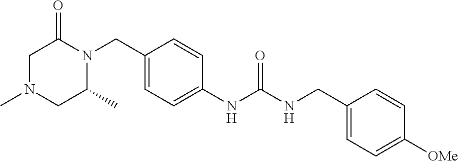 [SMILES: COc1ccc(CNC(=O)Nc2ccc(CN3C(=O)CN(C)C[C@H]3C)cc2)cc1] |
| | | | (S)-1-(4-((2,4-dimethyl-6-oxopiperazin-1- |
| | | | yl)methyl)phenyl)-3-(4-methoxybenzyl)urea. |
| | | | LCMS-APCI (POS.) m/z: 397 (M + H)⁺. ¹H NMR |
| | | | (300 MHz, DMSO-d₆) δ: 8.47 (s, 1H), 7.32 (d, J = |
| | | | 8.5 Hz, 2H), 7.20 (d, J = 8.6 Hz, 2H), 7.06 (d, J = 8.4 |
| | | | Hz, 2H), 6.96-6.84 (m, 2H), 6.47 (s, 1H), 4.93 (d, |
| | | | J = 14.9 Hz, 1H), 4.19 (d, J = 5.8 Hz, 2H), 3.96 (d, J = |
| | | | 15.0 Hz, 1H), 3.71 (s, 3H), 3.31 (s, 1H), 3.11 (d, J = |
| | | | 16.2 Hz, 1H), 2.83 (d, J = 16.3 Hz, 1H), 2.43 (d, J = |
| | | | 3.4 Hz, 2H), 2.17 (s, 3H), 1.15 (d, J = 6.3 Hz, 3H). |
| | | | (S)-1-(4-((2,4-dimethyl-6-oxopiperazin-1- |
| | | | yl)methyl)phenyl)-3-(4-methoxybenzyl)urea. |
| | | | LCMS-APCI (POS.) m/z: 397 (M + H)⁺. ¹H NMR |
| | | | (300 MHz, DMSO-d₆) δ: 8.47 (s, 1H), 7.32 (d, J = |
| | | | 8.5 Hz, 2H), 7.20 (d, J = 8.6 Hz, 2H), 7.06 (d, J = 8.4 |
| | | | Hz, 2H), 6.96-6.84 (m, 2H), 6.47 (s, 1H), 4.93 (d, J = |
| | | | 14.9 Hz, 1H), 4.19 (d, J = 5.8 Hz, 2H), 3.96 (d, J = |
| | | | 15.0 Hz, 1H), 3.71 (s, 3H), 3.31 (s, 1H), 3.11 (d, J = |
| | | | 16.2 Hz, 1H), 2.83 (d, J = 16.3 Hz, 1H), 2.43 (d, J = |
| | | | 3.4 Hz, 2H), 2.17 (s, 3H), 1.15 (d, J = 6.3 Hz, 3H). |
| 427 & 428 | 1-(4-chloro- benzyl)-3- (4-((5- methyl-2- oxopipera- zin-1- yl)methyl) phenyl)urea. | CHIRAL- PAK IG | 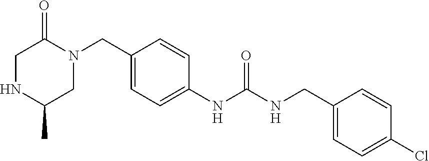 [SMILES: C[C@@H]1CN(Cc2ccc(NC(=O)NCc3ccc(Cl)cc3)cc2)C(=O)CN1] |
| | | | 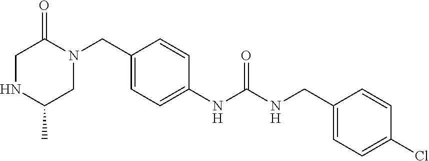 [SMILES: C[C@H]1CN(Cc2ccc(NC(=O)NCc3ccc(Cl)cc3)cc2)C(=O)CN1] |
| | | | (R)-1-(4-chlorobenzyl)-3-(4-((5-methyl-2- |
| | | | oxopiperazin-1-yl)methyl)phenyl)urea. LCMS- |
| | | | APCI (POS.) m/z: 387 (M + H)⁺. ¹H NMR (400 MHz, |
| | | | DMSO-d₆) δ 8.60 (s, 1H), 7.38 (dd, J = 8.4, 6.3 Hz, |
| | | | 3H), 7.36-7.27 (m, 3H), 7.10 (d, J = 8.5 Hz, 2H), |
| | | | 6.64 (t, J = 6.1 Hz, 1H), 4.46-4.32 (m, 2H), 4.28 (d, |
| | | | J = 5.9 Hz, 2H), 3.31 (s, 2H), 3.05 (dd, J = 10.8, 3.2 |
| | | | Hz, 1H), 2.95-2.77 (m, 2H), 2.55 (s, 1H), 0.97 (d, |
| | | | J = 6.0 Hz, 3H). |
| | | | (S)-1-(4-chlorobenzyl)-3-(4-((5-methyl-2- |
| | | | oxopiperazin-1-yl)methyl)phenyl)urea. LCMS- |
| | | | APCI (POS.) m/z: 387 (M + H) ¹H NMR (400 MHz, |
| | | | DMSO-d₆) δ 8.60 (s, 1H), 7.42-7.35 (m, 3H), 7.35- |
| | | | 7.29 (m, 3H), 7.10 (d, J = 8.3 Hz, 2H), 6.64 (t, J = 6.0 |
| | | | Hz, 1H), 4.46-4.32 (m, 2H), 4.28 (d, J = 5.9 Hz, |
| | | | 2H), 3.31 (s, 2H), 3.05 (dd, J = 10.9, 3.2 Hz, 1H), |
| | | | 2.97-2.78 (m, 2H), 2.55 (s, 1H), 0.97 (d, J = 6.0 Hz, 3H). |
| 429 & 430 | 1-(4-chloro- benzyl)-3- (4-((2- methyl-6- oxopipe- razin-1- yl)methyl) phenyl)urea. | | 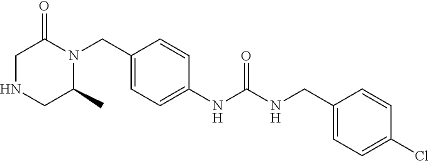 [SMILES: C[C@H]1CNCC(=O)N1Cc1ccc(NC(=O)NCc2ccc(Cl)cc2)cc1] |
| | | | 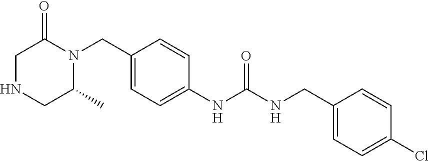 [SMILES: C[C@@H]1CNCC(=O)N1Cc1ccc(NC(=O)NCc2ccc(Cl)cc2)cc1] |
| | | | (S)-1-(4-chlorobenzyl)-3-(4-((2-methyl-6- |
| | | | oxopiperazin-1-yl)methyl)phenyl)urea. LCMS- |
| | | | APCI (POS.) m/z: 387 (M + H)⁺. ¹H NMR (400 MHz, |
| | | | DMSO-d₆) δ 8.51 (s, 1H), 7.32 (d, J = 8.4 Hz, 2H), |
| | | | 7.29-7.20 (m, 4H), 7.03 (d, J = 8.3 Hz, 2H), 6.56 (t, |
| | | | J = 6.0 Hz, 1H), 4.87 (d, J = 15.0 Hz, 1H), 4.20 (d, |
| | | | J = 6.0 Hz, 2H), 3.90 (d, J = 15.0 Hz, 1H), 3.24-3.10 |
| | | | (m, 3H), 2.79 (dd, J = 13.0, 4.2 Hz, 2H), 2.57 (dd, J = |
| | | | 13.0, 4.1 Hz, 1H), 1.07 (d, J = 6.4 Hz, 3H). |
| | | | (R)-1-(4-chlorobenzyl)-3-(4-((2-methyl-6- |
| | | | oxopiperazin-1-yl)methyl)phenyl)urea. LCMS- |
| | | | APCI (POS.) m/z: 387 (M + H)⁺. ¹H NMR (400 MHz, |
| | | | DMSO-d₆) δ 8.51 (s, 1H), 7.32 (d, J = 8.4 Hz, 2H), |
| | | | 7.29-7.20 (m, 4H), 7.03 (d, J = 8.4 Hz, 2H), 6.56 (t, |
| | | | J = 6.0 Hz, 1H), 4.87 (d, J = 15.0 Hz, 1H), 4.20 (d, |
| | | | J = 6.0 Hz, 2H), 3.90 (d, J = 15.0 Hz, 1H), 3.24-3.14 |
| | | | (m, 3H), 2.79 (dd, J = 13.0, 4.2 Hz, 2H), 2.57 (dd, J = |
| | | | 13.0, 4.1 Hz, 1H), 1.07 (d, J = 6.4 Hz, 3H). |
| 404 & 405 | 1-(4-chloro- benzyl)-3- (4-(1-(4- methyl-2- oxopipera- zin-1- yl)ethyl) phenyl)urea | CHIRAL- PAK IG | 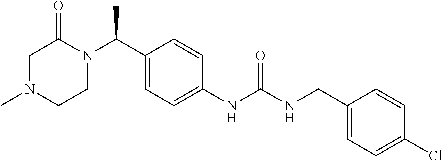 [SMILES: C[C@@H](c1ccc(NC(=O)NCc2ccc(Cl)cc2)cc1)N1CCN(C)CC1=O] |
| | | | 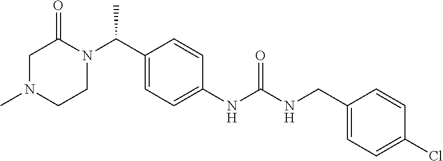 [SMILES: C[C@H](c1ccc(NC(=O)NCc2ccc(Cl)cc2)cc1)N1CCN(C)CC1=O] |
| | | | (S)-1-(4-chlorobenzyl)-3-(4-(1-(4-methyl-2- |
| | | | oxopiperazin-1-yl)ethyl)phenyl)urea. LCMS-APCI |
| | | | (POS.) m/z: 401 (M + H)⁺. ¹H NMR (300 MHz, |
| | | | DMSO-d₆) δ: 8.62 (s, 1H), 7.38 (tt, J = 6.3, 2.7 Hz, |
| | | | 4H), 7.34-7.28 (m, 2H), 7.15-7.09 (m, 2H), 6.64 |
| | | | (t, J = 6.0 Hz, 1H), 5.77 (q, J = 7.1 Hz, 1H), 4.28 (d, |
| | | | J = 6.0 Hz, 2H), 3.16 (ddd, J = 11.5, 7.3, 4.2 Hz, 1H), |
| | | | 3.03 (d, J = 16.3 Hz, 1H), 2.95 (d, J = 16.3 Hz, 1H), |
| | | | 2.69 (ddd, J = 11.6, 6.0, 4.2 Hz, 1H), 2.56 (dd, J = |
| | | | 11.6, 5.1 Hz, 1H), 2.39 (ddd, J = 11.6, 7.2, 4.2 Hz, |
| | | | 1H), 2.18 (s, 3H), 1.40 (d, J = 7.2 Hz, 3H). |
| | | | (R)-1-(4-chlorobenzyl)-3-(4-(1-(4-methyl-2- |
| | | | oxopiperazin-1-yl)ethyl)phenyl)urea. LCMS-APCI |
| | | | (POS.) m/z: 401 (M + H)⁺. ¹H NMR (300 MHz, |
| | | | DMSO-d₆) δ: 8.62 (s, 1H), 7.43-7.34 (m, 4H), 7.32 |
| | | | (d, J = 8.5 Hz, 2H), 7.15-7.09 (m, 2H), 6.64 (t, J = |
| | | | 6.0 Hz, 1H), 5.77 (q, J = 7.1 Hz, 1H), 4.28 (d, J = 5.9 |
| | | | Hz, 2H), 3.16 (ddd, J = 11.6, 7.2, 4.2 Hz, 1H), 3.03 |
| | | | (d, J = 16.3 Hz, 1H), 2.95 (d, J = 16.3 Hz, 1H), 2.69 |
| | | | (ddd, J = 11.7, 6.0, 4.2 Hz, 1H), 2.56 (dd, J = 11.5, |
| | | | 5.1Hz, 1H), 2.39 (ddd, J = 11.6, 7.3, 4.2 Hz, 1H), |
| | | | 2.18 (s, 3H), 1.40 (d, J = 7.2 Hz, 3H). |
| 406 | 1-(4-chloro- benzyl)-3- (4((methyl (2-oxopy- rrolidin- 3-yl)amino) methyl) phenyl)urea (Inter- mediate 27.1) | CHIRAL- PAK AD-H | 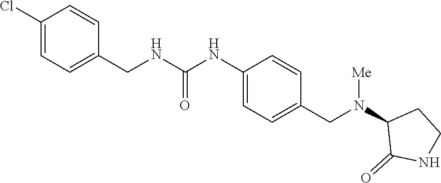 [SMILES: CN(Cc1ccc(NC(=O)NCc2ccc(Cl)cc2)cc1)[C@H]1CCNC1=O] |
| | | | 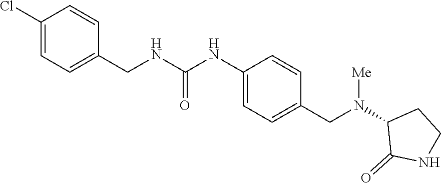 [SMILES: CN(Cc1ccc(NC(=O)NCc2ccc(Cl)cc2)cc1)[C@@H]1CCNC1=O] |
| | | | (S)-1-(4-chlorobenzyl)-3-(4-((methyl(2- |
| | | | oxopyrrolidin-3-yl)amino)methyl)phenyl)urea. |
| | | | LCMS-APCI (POS.) m/z: 387 (M + H)⁺. ¹H NMR |
| | | | (300 MHz, DMSO-d₆) δ 8.55 (s, 1H), 7.68 (s, 1H), |
| | | | 7.46-7.36 (m, 2H), 7.36-7.29 (m, 4H), 7.17 (d, J = |
| | | | 8.2 Hz, 2H), 6.62 (t, J = 6.0 Hz, 1H), 4.28 (d, J = 5.9 |
| | | | Hz, 2H), 3.57 (s, 2H), 3.37 (d, J = 8.8 Hz, 1H), 3.21- |
| | | | 3.04 (m, 2H), 2.14 (s, 3H), 2.07-2.02 (m, 1H), 2.02- |
| | | | 1.89 (m, 1H). |
| | | | (S)-1-(4-chlorobenzyl)-3-(4-((methyl(2- |
| | | | oxopyrrolidin-3-yl)amino)methyl)phenyl)urea. |
| | | | LCMS-APCI (POS.) m/z: 387 (M + H)⁺. ¹H NMR |
| | | | (300 MHz, DMSO-d₆) δ 8.55 (s, 1H), 7.68 (s, 1H), |
| | | | 7.45-7.25 (m, 6H), 7.17 (d, J = 8.4 Hz, 2H), 6.62 (t, |
| | | | J = 6.0 Hz, 1H), 4.28 (d, J = 6.0 Hz, 2H), 3.57 (s, |
| | | | 2H), 3.37 (d, J = 8.9 Hz, 1H), 3.21-3.04 (m, 2H), |
| | | | 2.14 (s, 3H), 2.04 (td, J = 10.4, 9.7, 5.5 Hz, 1H), 2.00- |
| | | | 1.91 (m, 1H). |
| 408 & 409 | 1-(4-chloro- benzyl)-3- (4-((methyl (1-methyl- 2-oxopyrro- lidin-3-yl) amino) methyl) phenyl)urea (interme- diate 27.2) | CHIRAL- PAK AD-H | 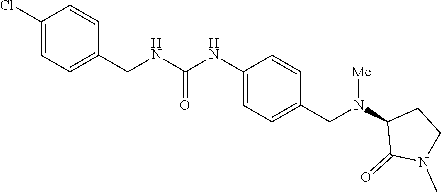 [SMILES: CN1CC[C@H](N(C)Cc2ccc(NC(=O)NCc3ccc(Cl)cc3)cc2)C1=O] |
| | | | 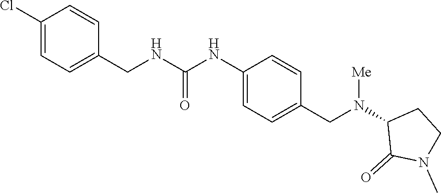 [SMILES: CN1CC[C@@H](N(C)Cc2ccc(NC(=O)NCc3ccc(Cl)cc3)cc2)C1=O] |
| | | | (S)-1-(4-chlorobenzyl)-3-(4-((methyl(1-methyl-2- |
| | | | oxopyrrolidin-3-yl)amino)methyl)phenyl)urea. |
| | | | LCMS-APCI (POS.) m/z: 401 (M + H)⁺. ¹H NMR |
| | | | (300 MHz, DMSO-d₆) δ 8.58 (s, 1H), 7.43-7.36 (m, |
| | | | 2H), 7.36-7.29 (m, 4H), 7.16 (d, J = 8.4 Hz, 2H), |
| | | | 6.66 (t, J = 6.0 Hz, 1H), 4.28 (d, J = 5.9 Hz, 2H), 3.57 |
| | | | (s, 2H), 3.44 (t, J = 8.7 Hz, 1H), 3.30-3.14 (m, 2H), |
| | | | 2.72 (s, 3H), 2.12 (s, 3H), 2.03 (ddt, J = 12.0, 8.8, 4.6 |
| | | | Hz, 1H), 1.90 (dq, J = 12.8, 8.5 Hz, 1H). |
| | | | (R)-1-(4-chlorobenzyl)-3-(4-((methyl(1-methyl-2- |
| | | | oxopyrrolidin-3-yl)amino)methyl)phenyl)urea. |
| | | | LCMS-APCI (POS.) m/z: 401 (M + H)⁺. ¹H NMR |
| | | | (300 MHz, DMSO-d₆) δ 8.57 (s, 1H), 7.43-7.36 (m, |
| | | | 2H), 7.36-7.29 (m, 4H), 7.16 (d, J = 8.4 Hz, 2H), |
| | | | 6.64 (t, J = 6.1 Hz, 1H), 4.28 (d, J = 5.9 Hz, 2H), 3.57 |
| | | | (s, 2H), 3.44 (t, J = 8.7 Hz, 1H), 3.32-3.23 (m, 1H), |
| | | | 3.19 (dt, J = 9.6, 7.7 Hz, 1H), 2.72 (s, 3H), 2.12 (s, |
| | | | 3H), 2.03 (dtd, J = 11.9, 8.3, 7.1, 3.9 Hz, 1H), 1.90 |
| | | | (dq, J = 12.9, 8.6 Hz, 1H). |
| 410 | 1-(4-fluoro- benzyl)-3- ((methyl(1- methyl-2- oxopyrro- lidin-3-yl) amino) methyl) phenyl)urea (Interme- diate 27.3 | CHIRAL- PAK AD-H | 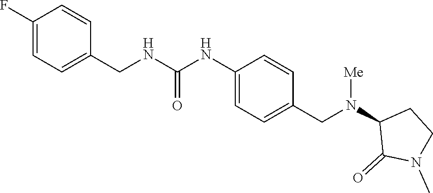 [SMILES: CN1CC[C@H](N(C)Cc2ccc(NC(=O)NCc3ccc(F)cc3)cc2)C1=O] |
| | | | 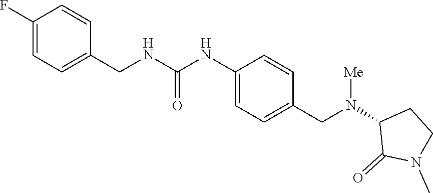 [SMILES: CN1CC[C@@H](N(C)Cc2ccc(NC(=O)NCc3ccc(F)cc3)cc2)C1=O] |
| | | | (S)-1-(4-fluorobenzyl)-3-(4-((methyl(1-methyl-2- |
| | | | oxopyrrolidin-3-yl)amino)methyl)phenyl)urea. |
| | | | LCMS-APCI (POS.) m/z: 385 (M + H)⁺. ¹H NMR |
| | | | (300 MHz, DMSO-d₆) δ 8.54 (s, 1H), 7.39-7.30 (m, |
| | | | 4H), 7.21-7.10 (m, 4H), 6.61 (t, J = 6.0 Hz, 1H), |
| | | | 4.27 (d, J = 5.9 Hz, 2H), 3.59 (s, 2H), 3.44 (t, J = 8.7 |
| | | | Hz, 1H), 3.32-3.23 (m, 1H), 3.23-3.15 (m, 1H), |
| | | | 2.72 (s, 3H), 2.12 (s, 3H), 2.10-1.97 (m, 1H), 1.90 |
| | | | (dq, J = 12.8, 8.5 Hz, 1H). |
| | | | (R)-1-(4-fluorobenzyl)-3-(4-((methyl(1-methyl-2- |
| | | | oxopyrrolidin-3-yl)amino)methyl)phenyl)urea. |
| | | | LCMS-APCI (POS.) m/z: 385 (M + H)⁺. ¹H NMR |
| | | | (300 MHz, DMSO-d₆) δ 8.55 (s, 1H), 7.34 (dt, J = |
| | | | 8.8, 2.9 Hz, 4H), 7.21-7.10 (m, 4H), 6.63 (t, J = 6.0 |
| | | | Hz, 1H), 4.27 (d, J = 5.9 Hz, 2H), 3.57 (s, 2H), 3.44 |
| | | | (t, J = 8.7 Hz, 1H), 3.27 (td, J = 9.3, 3.1 Hz, 1H), 3.23- |
| | | | 3.15 (m, 1H), 2.72 (s, 3H), 2.12 (s, 3H), 2.10-1.97 |
| | | | (m, 1H), 1.90 (dq, J = 12.8, 8.3 Hz, 1H). |
| 411 | 1-(4- methoxy- benzyl)-3- (4-((methyl (1-methyl- 2-oxopyrro- lidin-3- yl)amino) methyl) phenyl)urea (Interme- diate 27.4) | CHIRAL- PAK AD-H | 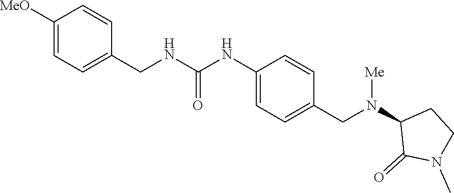 [SMILES: COc1ccc(CNC(=O)Nc2ccc(CN(C)[C@H]3CCN(C)C3=O)cc2)cc1] |
| | | | 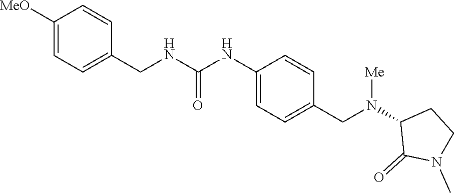 [SMILES: COc1ccc(CNC(=O)Nc2ccc(CN(C)[C@@H]3CCN(C)C3=O)cc2)cc1] |
| | | | (S)-1-(4-methoxybenzyl)-3-(4-((methyl(1-methyl-2- |
| | | | oxopyrrolidin-3-yl)amino)methyl)phenyl)urea. |
| | | | LCMS-APCI (POS.) m/z: 397 (M + H)⁺. ¹H NMR |
| | | | (300 MHz, DMSO-d₆) δ 8.46 (s, 1H), 7.37-7.29 (m, |
| | | | 2H), 7.27-7.19 (m, 2H), 7.19-7.13 (m, 2H), 6.94- |
| | | | 6.86 (m, 2H), 6.49 (t, J = 5.9 Hz, 1H), 4.22 (d, J = 5.8 |
| | | | Hz, 2H), 3.73 (s, 3H), 3.57 (s, 2H), 3.44 (t, J = 8.7 |
| | | | Hz, 1H), 3.27 (td, J = 9.4, 3.1Hz, 1H), 3.19 (dt, J = |
| | | | 9.5, 7.7 Hz, 1H), 2.72 (s, 3H), 2.12 (s, 3H), 2.10- |
| | | | 1.97 (m, 1H), 1.90 (dq, J = 12.8, 8.6 Hz, 1H). |
| | | | (R)-1-(4-methoxybenzyl)-3-(4-((methyl(1-methyl- |
| | | | 2-oxopyrrolidin-3-yl)amino)methyl)phenyl)urea. |
| | | | LCMS-APCI (POS.) m/z: 397 (M + H) ¹H NMR |
| | | | (300 MHz, DMSO-d₆) δ 8.47 (s, 1H), 7.37-7.29 (m, |
| | | | 2H), 7.27-7.19 (m, 2H), 7.23-7.13 (m, 2H), 6.95- |
| | | | 6.86 (m, 2H), 6.50 (t, J = 5.9 Hz, 1H), 4.22 (d, J = 5.8 |
| | | | Hz, 2H), 3.73 (s, 3H), 3.57 (s, 2H), 3.44 (t, J = 8.7 |
| | | | Hz, 1H), 3.27 (td, J = 9.3, 3.1Hz, 1H), 3.19 (dt, J = |
| | | | 9.6, 7.8 Hz, 1H), 2.72 (s, 3H), 2.12 (s, 3H), 2.09- |
| | | | 1.97 (m, 1H), 1.90 (dq, J = 12.8, 8.2 Hz, 1H). |
| 403 | 1-(4-chloro- benzyl)-3- (4-(2- oxooxazo- lidin-5-yl) phenyl)urea | CHIRAL- PAK IG | 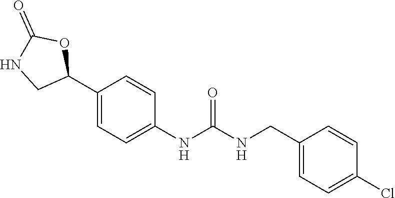 [SMILES: O=C(NCc1ccc(Cl)cc1)Nc1ccc([C@H]2CNC(=O)O2)cc1] |
| | | | 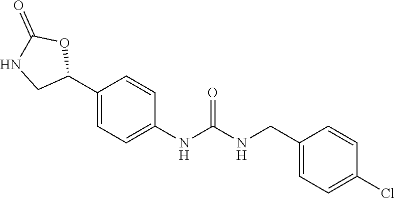 [SMILES: O=C(NCc1ccc(Cl)cc1)Nc1ccc([C@@H]2CNC(=O)O2)cc1] |
| | | | (S)-1-(4-chlorobenzyl)-3-(4-(2-oxooxazolidin-5- |
| | | | yl)phenyl)urea. LCMS-APCI (POS.) m/z: 346 (M + H)⁺. |
| | | | ¹H NMR (300 MHz, MeOD-d₄) δ 7.49-7.42 (m, 2H), |
| | | | 7.37-7.30 (m, 6H), 5.62 (t, J = 8.1 Hz, 1H), 4.39 (s, 2H), |
| | | | 3.96 (t, J = 8.9 Hz, 1H), 3.51 (dd, J = 9.1, 7.6 Hz, 1H). |
| | | | (R)-1-(4-chlorobenzyl)-3-(4-(2-oxooxazolidin-5-yl) |
| | | | phenyl)urea. LCMS-APCI (POS.) m/z: 346 (M + H)⁺. |
| | | | ¹H NMR (300 MHz, MeOD-d₄) δ 7.46 (d, J = 8.6 Hz, |
| | | | 2H), 7.33 (d, J = 6.9 Hz, 6H), 5.66-5.57 (m, 1H), 4.39 |
| | | | (s, 2H), 3.96 (t, J = 8.9 Hz, 1H), 3.51 (dd, J = 9.0, 7.6 Hz, 1H). |
| 407 | 1-(4-chloro- benzyl)-3- (4-(3- methyl- 2-oxooxazo- lidin-5-yl) phenyl)urea | CHIRAL- PAK IF | 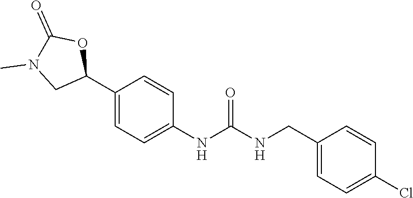 [SMILES: CN1C[C@H](c2ccc(NC(=O)NCc3ccc(Cl)cc3)cc2)OC1=O] |
| | | | 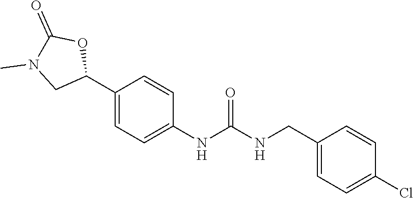 [SMILES: CN1C[C@@H](c2ccc(NC(=O)NCc3ccc(Cl)cc3)cc2)OC1=O] |
| | | | (S)-1-(4-chlorobenzyl)-3-(4-(3-methyl-2- |
| | | | oxooxazolidin-5-yl)phenyl)urea. LCMS-APCI |
| | | | (POS.) m/z: 360 (M + H)⁺. ¹H NMR (300 MHz, |
| | | | MeOD-d₄) δ 7.49-7.42 (m, 2H), 7.39-7.29 (m, |
| | | | 6H), 5.55-5.47 (m, 1H), 4.39 (s, 2H), 3.97 (t, J = 8.9 |
| | | | Hz, 1H), 3.53 (dd, J = 9.0, 7.6 Hz, 1H), 2.93 (s, 3H). |
| | | | (R)-1-(4-chlorobenzyl)-3-(4-(3-methyl-2- |
| | | | oxooxazolidin-5-yl)phenyl)urea. LCMS-APCI |
| | | | (POS.) m/z: 360 (M + H)⁺. ¹H NMR (300 MHz, |
| | | | MeOD-d₄) δ 7.49-7.42 (m, 2H), 7.39-7.29 (m, |
| | | | 6H), 5.51 (t, J = 8.2 Hz, 1H), 4.39 (s, 2H), 3.97 (t, J = |
| | | | 8.9 Hz, 1H), 3.53 (dd, J = 9.0, 7.6 Hz, 1H), 2.93 (s, 3H). |
| 413 | 1-(4- methoxy- benzyl)- 3-(4-(3- methyl-2- oxooxazo- lidin-5-yl) phenyl)urea | CHIRAL- PAK IG | 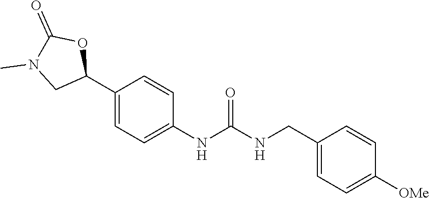 [SMILES: COc1ccc(CNC(=O)Nc2ccc([C@H]3CN(C)C(=O)O3)cc2)cc1] |
| | | | 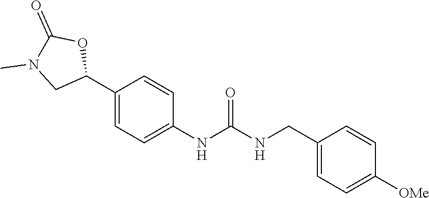 [SMILES: COc1ccc(CNC(=O)Nc2ccc([C@@H]3CN(C)C(=O)O3)cc2)cc1] |
| | | | (S)-1-(4-methoxybenzyl)-3-(4-(3-methyl-2- |
| | | | oxooxazolidin-5-yl)phenyl)urea LCMS-APCI |
| | | | (POS.) m/z: 360 (M + H)⁺. ¹H NMR (300 MHz, |
| | | | MeOD-d₄) δ 7.48-7.41 (m, 2H), 7.36-7.23 (m, 4H), |
| | | | 6.94-6.86 (m, 2H), 5.51 (dd, J = 8.7, 7.7 Hz, 1H), 4.33 (s, |
| | | | 2H), 3.97 (t, J = 8.9 Hz, 1H), 3.79 (s, 3H), 3.53 (dd, J = |
| | | | 9.0, 7.6 Hz, 1H), 2.93 (s, 3H). |
| | | | (R)-1-(4-methoxybenzyl)-3-(4-(3-methyl-2- |
| | | | oxooxazolidin-5-yl)phenyl)urea LCMS-APCI |
| | | | (POS.) m/z: 360 (M + H)⁺. ¹H NMR (300 MHz, |
| | | | MeOD-d₄) δ 7.49-7.41 (m, 2H), 7.36-7.22 (m, 4H), |
| | | | 6.94-6.86 (m, 2H), 5.55-5.46 (m, 1H), 4.33 (s, 2H), |
| | | | 3.97 (t, J = 8.9 Hz, 1H), 3.79 (s, 3H), 3.53 (dd, J = 9.0, 7.6 |
| | | | Hz, 1H), 2.93 (s, 3H). |
| 412 | 1-(4- fluoro- benzyl)-3- (4-(3- methyl-2- oxooxazo- lidin-5-yl) phenyl)urea | CHIRAL- PAK IG | 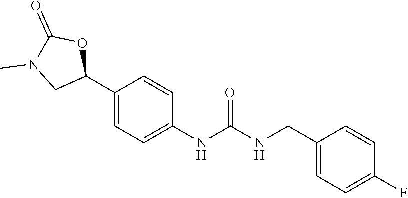 [SMILES: CN1C[C@H](c2ccc(NC(=O)NCc3ccc(F)cc3)cc2)OC1=O] |
| | | | 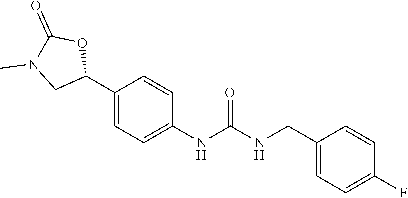 [SMILES: CN1C[C@@H](c2ccc(NC(=O)NCc3ccc(F)cc3)cc2)OC1=O] |
| | | | (S)-1-(4-fluorobenzyl)-3-(4-(3-methyl-2- |
| | | | oxooxazolidin-5-yl)phenyl)urea. LCMS-APCI |
| | | | (POS.) m/z: 344 (M + H)⁺. ¹H NMR (300 MHz, |
| | | | MeOD-d₄) δ 7.49-7.42 (m, 2H), 7.40-7.29 (m, 4H), |
| | | | 7.12-7.02 (m, 2H), 5.51 (dd, J = 8.7, 7.6 Hz, 1H), 4.39 |
| | | | (s, 2H), 3.97 (t, J = 8.9 Hz, 1H), 3.53 (dd, J = 9.0, 7.6 Hz, |
| | | | 1H), 2.93 (s, 3H). |
| | | | (R)-1-(4-fluorobenzyl)-3-(4-(3-methyl-2- |
| | | | oxooxazolidin-5-yl)phenyl)urea. LCMS-APCI |
| | | | (POS.) m/z: 344 (M + H)⁺. ¹H NMR (300 MHz, |
| | | | MeOD-d₄) δ 7.49-7.42 (m, 2H), 7.40-7.29 (m, 4H), |
| | | | 7.12-7.02 (m, 2H), 5.55-5.46 (m, 1H), 4.38 (s, 2H), |
| | | | 3.97 (t, J = 8.9 Hz, 1H), 3.53 (dd, J = 9.0, 7.6 Hz, 1H), |
| | | | 2.93 (s, 3H). |
| 417 & 418 | 1-(4- chloro- benzyl)-3- (4-((2-oxo- 5-(pyridin- 3-yl) pyrrolidin- 1-yl) methyl) phenyl)urea | CHIRAL- PAK IA | 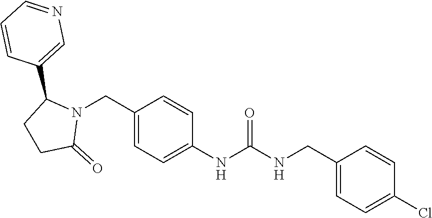 [SMILES: O=C(NCc1ccc(Cl)cc1)Nc1ccc(CN2C(=O)CC[C@H]2c2cccnc2)cc1] |
| | | | 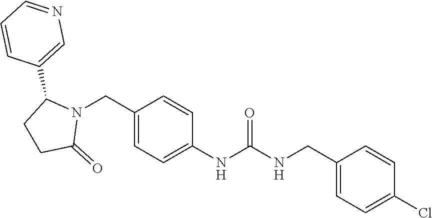 [SMILES: O=C(NCc1ccc(Cl)cc1)Nc1ccc(CN2C(=O)CC[C@@H]2c2cccnc2)cc1] |
| | | | (S)-1-(4-chlorobenzyl)-3-(4-((2-oxo-5-(pyridin-3- |
| | | | yl)pyrrolidin-1-yl)methyl)phenyl)urea. LCMS- |
| | | | APCI (POS.) m/z: 435 (M + H)⁺. ¹H NMR (300 MHz, |
| | | | DMSO-d₆) δ 8.66-8.48 (m, 2H), 8.38 (d, J = 2.3 |
| | | | Hz, 1H), 7.63 (dt, J = 7.9, 2.0 Hz, 1H), 7.42-7.35 |
| | | | (m, 3H), 7.35-7.25 (m, 4H), 6.93-6.83 (m, 2H), |
| | | | 6.63 (t, J = 6.0 Hz, 1H), 4.72 (d, J = 14.9 Hz, 1H), |
| | | | 4.49 (dd, J = 8.1, 5.6 Hz, 1H), 4.28 (d, J = 6.0 Hz, |
| | | | 2H), 3.43 (d, J = 14.9 Hz, 1H), 2.54 (s, 1H), 2.49- |
| | | | 2.31 (m, 2H), 1.83 (td, J = 10.7, 5.3 Hz, 1H). |
| | | | (R)-1-(4-chlorobenzyl)-3-(4-((2-oxo-5-(pyridin-3- |
| | | | yl)pyrrolidin-1-yl)methyl)phenyl)urea. LCMS- |
| | | | APCI (POS.) m/z: 435 (M + H)⁺. ¹H NMR (300 MHz, |
| | | | DMSO-d₆) δ 8.69-8.45 (m, 2H), 8.43-8.34 (m, |
| | | | 1H), 7.63 (dt, J = 7.9, 1.9 Hz, 1H), 7.39 (dt, J = 8.5, |
| | | | 2.3 Hz, 3H), 7.33-7.25 (m, 4H), 6.87 (d, J = 8.5 Hz, |
| | | | 2H), 6.63 (t, J = 6.1 Hz, 1H), 4.72 (d, J = 14.9 Hz, |
| | | | 1H), 4.49 (t, J = 6.8 Hz, 1H), 4.28 (d, J = 6.0 Hz, |
| | | | 2H), 3.43 (d, J = 14.9 Hz, 1H), 2.54 (s, 1H), 2.49- |
| | | | 2.28 (m, 2H), 1.83 (td, J = 10.4, 5.2 Hz, 1H). |
| 419 | 1-(4- chloro- benzyl)-3- (4-((2-(5- fluoropy- ridin-3-yl)- 5-oxopyrro- lidin-1- yl)methyl) phenyl)urea | CHIRAL- PAK IE | 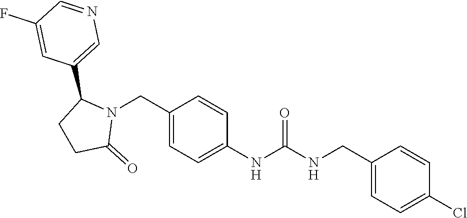 [SMILES: O=C(NCc1ccc(Cl)cc1)Nc1ccc(CN2C(=O)CC[C@H]2c2cncc(F)c2)cc1] |
| | | | 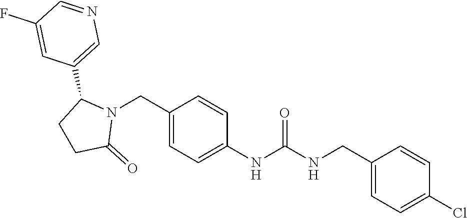 [SMILES: O=C(NCc1ccc(Cl)cc1)Nc1ccc(CN2C(=O)CC[C@@H]2c2cncc(F)c2)cc1] |
| | | | (S)-1-(4-chlorobenzyl)-3-(4-((2-(5-fluoropyridin-3-yl)-5- |
| | | | oxopyrrolidin-1-yl)methyl)phenyl)urea. LCMS-APCI |
| | | | (POS.) m/z: 453 (M + H)⁺. ¹H NMR (300 MHz, |
| | | | DMSO-d₆) δ 8.59 (s, 1H), 8.51 (d, J = 2.8 Hz, 1H), |
| | | | 8.26 (t, J = 1.8 Hz, 1H), 7.60 (ddd, J = 9.8, 2.8, 1.7 |
| | | | Hz, 1H), 7.43-7.34 (m, 2H), 7.37-7.26 (m, 4H), |
| | | | 6.92-6.84 (m, 2H), 6.63 (t, J = 6.1 Hz, 1H), 4.66 (d, |
| | | | J = 14.9 Hz, 1H), 4.56 (t, J = 6.7 Hz, 1H), 4.28 (d, J = |
| | | | 5.9 Hz, 2H), 3.56 (d, J = 14.9 Hz, 1H), 2.62-2.51 |
| | | | (m, 1H), 2.48-2.34 (m, 2H), 1.90-1.78 (m, 1H). |
| | | | (R)-1-(4-chlorobenzyl)-3-(4-((2-(5-fluoropyridin-3-yl)- |
| | | | 5-oxopyrrolidin-1-yl)methyl)phenyl)urea. LCMS- |
| | | | APCI (POS.) m/z: 453 (M + H)⁺. ¹H NMR (300 MHz, |
| | | | DMSO-d₆) δ 8.60 (s, 1H), 8.51 (d, J = 2.7 Hz, 1H), |
| | | | 8.26 (t, J = 1.8 Hz, 1H), 7.64-7.56 (m, 1H), 7.43- |
| | | | 7.36 (m, 2H), 7.36-7.27 (m, 4H), 6.92-6.84 (m, |
| | | | 2H), 6.64 (t, J = 6.0 Hz, 1H), 4.66 (d, J = 14.9 Hz, |
| | | | 1H), 4.56 (t, J = 6.7 Hz, 1H), 4.28 (d, J = 6.0 Hz, 2H), |
| | | | 3.56 (d, J = 14.9 Hz, 1H), 2.62-2.51 (m, 1H), 2.48- |
| | | | 2.34 (m, 2H), 1.90-1.78 (m, 1H). |
| 451 | 1-(4-chloro- benzyl)-3- (4-((3,4- dimethyl-2- oxopipe- razin-1- yl)methyl) phenyl)urea. | CHIRAL ART Cellulose- SC | 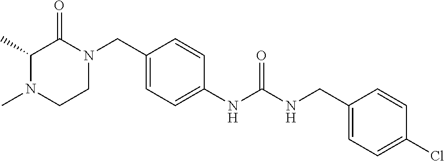 [SMILES: C[C@@H]1C(=O)N(Cc2ccc(NC(=O)NCc3ccc(Cl)cc3)cc2)CCN1C] |
| | | | 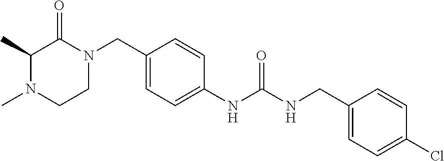 [SMILES: C[C@H]1C(=O)N(Cc2ccc(NC(=O)NCc3ccc(Cl)cc3)cc2)CCN1C] |
| | | | (R)-1-(4-chlorobenzyl)-3-(4-((3,4-dimethyl-2- |
| | | | oxopiperazin-1-yl)methyl)phenyl)urea. LCMS- |
| | | | APCI (POS.) m/z: 401 (M + H)⁺. ¹H NMR (300 MHz, |
| | | | DMSO-d₆) δ 8.60 (s, 1H), 7.43-7.28 (m, 6H), 7.11- |
| | | | 7.05 (m, 2H), 6.64 (t, J = 6.0 Hz, 1H), 4.43 (d, J = |
| | | | 14.5 Hz, 1H), 4.36 (d, J = 14.5 Hz, 1H), 4.28 (d, J = |
| | | | 6.0 Hz, 2H), 3.26-3.18 (m, 1H), 3.05 (dt, J = 11.7, |
| | | | 3.7 Hz, 1H), 2.84 (dt, J = 12.0, 4.0 Hz, 1H), 2.76 (q, J = |
| | | | 6.7 Hz, 1H), 2.38 (ddd, J = 12.1, 10.1, 4.1 Hz, 1H), |
| | | | 2.24 (s, 3H), 1.27 (d, J = 6.7 Hz, 3H). |
| | | | (S)-1-(4-chlorobenzyl)-3-(4-((3,4-dimethyl-2- |
| | | | oxopiperazin-1-yl)methyl)phenyl)urea. LCMS- |
| | | | APCI (POS.) m/z: 401 (M + H)⁺. ¹H NMR (300 MHz, |
| | | | DMSO-d₆) δ 8.59 (s, 1H), 7.43-7.28 (m, 6H), 7.11- |
| | | | 7.05 (m, 2H), 6.64 (t, J = 6.0 Hz, 1H), 4.43 (d, J = |
| | | | 14.5 Hz, 1H), 4.36 (d, J = 14.5 Hz, 1H), 4.28 (d, J = |
| | | | 6.0 Hz, 2H), 3.22 (dd, J = 10.3, 4.1 Hz, 1H), 3.05 (dt, |
| | | | J = 11.8, 3.7 Hz, 1H), 2.84 (dt, J = 12.1, 3.9 Hz, 1H), |
| | | | 2.76 (q, J = 6.7 Hz, 1H), 2.38 (ddd, J = 12.0, 10.1, 4.0 |
| | | | Hz, 1H), 2.24 (s, 3H), 1.27 (d, J = 6.7 Hz, 3H). |
| 455 | 1-(4-chloro- benzyl)-3- (4-(1-(2- oxopipe- ridin-1- yl)ethyl) phenyl)urea | CHIRAL- PAK IA | 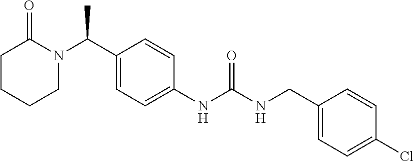 [SMILES: C[C@@H](c1ccc(NC(=O)NCc2ccc(Cl)cc2)cc1)N1CCCCC1=O] |
| | | | 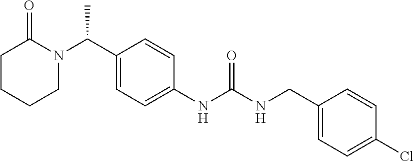 [SMILES: C[C@H](c1ccc(NC(=O)NCc2ccc(Cl)cc2)cc1)N1CCCCC1=O] |
| | | | (S)-1-(4-chlorobenzyl)-3-(4-(1-(2-oxopiperidin-1- |
| | | | yl)ethyl)phenyl)urea. LCMS-APCI (POS.) m/z: 386 |
| | | | (M + H)⁺. ¹H NMR (300 MHz, DMSO-d₆) δ 8.60 (s, |
| | | | 1H), 7.73-7.22 (m, 6H), 7.11 (d, J = 8.3 Hz, 2H), 6.63 (t, |
| | | | J = 6.1 Hz, 1H), 5.83 (q, J = 7.1 Hz, 1H), 4.28 (d, J = 6.0 |
| | | | Hz, 2H), 3.13-3.00 (m, 1H), 2.71-2.67 (m, 1H), 2.31 (d, |
| | | | J = 6.6 Hz, 2H), 1.65 (s, 3H), 1.59-1.51 (m, 1H), 1.38 (d, |
| | | | J = 7.1 Hz, 3H). |
| | | | (R)-1-(4-chlorobenzyl)-3-(4-(1-(2-oxopiperidin-1- |
| | | | yl)ethyl)phenyl)urea. LCMS-APCI (POS.) m/z: 386 |
| | | | (M + H)⁺. ¹H NMR (300 MHz, DMSO-d₆) δ 8.60 (s, |
| | | | 1H), 7.44-7.27 (m, 6H), 7.11 (d, J = 8.3 Hz, 2H), 6.63 (t, |
| | | | J = 6.0 Hz, 1H), 5.83 (q, J = 7.1 Hz, 1H), 4.28 (d, J = 5.9 |
| | | | Hz, 2H), 3.14-3.02 (m, 1H), 2.69 (dt, J = 11.8, 5.0 Hz, |
| | | | 1H), 2.30 (t, J = 6.3 Hz, 2H), 1.66 (s, 3H), 1.54 (dd, J = |
| | | | 13.3, 7.9 Hz, 1H), 1.38 (d, J = 7.1 Hz, 3H). |
| 443 | 1-(4-chloro- benzyl)-3- (4-((2- methyl-6- oxopipe- ridin-1- yl)methyl) phenyl)urea. | CHIRAL- PAK IC | 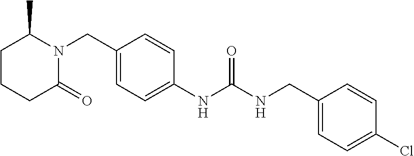 [SMILES: C[C@@H]1CCCC(=O)N1Cc1ccc(NC(=O)NCc2ccc(Cl)cc2)cc1] |
| | | | 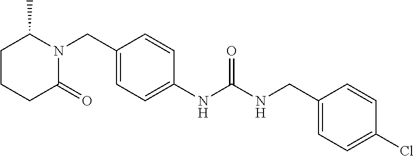 [SMILES: C[C@H]1CCCC(=O)N1Cc1ccc(NC(=O)NCc2ccc(Cl)cc2)cc1] |
| | | | (R)-1-(4-chlorobenzyl)-3-(4-((2-methyl-6- |
| | | | oxopiperidin-1-yl)methyl)phenyl)urea. LCMS- |
| | | | APCI (POS.) m/z: 386 (M + H)⁺. ¹H NMR (300 |
| | | | MHz, DMSO-d₆) δ 8.56 (s, 1H), 7.42-7.31 (m, 6H), |
| | | | 7.08 (d, J = 8.5 Hz, 2H), 6.62 (t, J = 6.0 Hz, 1H), 4.95 (d, |
| | | | J = 15.0 Hz, 1H), 4.28 (d, J = 5.8 Hz, 2H), 3.99 (d, J = 15.0 |
| | | | Hz, 1H), 3.37 (s, 1H), 2.29 (d, J = 6.9 Hz, 2H), 1.90-1.69 |
| | | | (m, 2H), 1.60 (ddd, J = 16.0, 12.7, 6.8 Hz, 2H), 1.14 (d, |
| | | | J = 6.4 Hz, 3H). |
| | | | (S)-1-(4-chlorobenzyl)-3-(4-((2-methyl-6- |
| | | | oxopiperidin-1-yl)methyl)phenyl)urea. LCMS- |
| | | | APCI (POS.) m/z: 386 (M + H)⁺. ¹H NMR (300 |
| | | | MHz, DMSO-d₆) δ 8.56 (s, 1H), 7.53-7.22 (m, 6H), |
| | | | 7.08 (d, J = 8.5 Hz, 2H), 6.62 (t, J = 6.0 Hz, 1H), 4.95 (d, |
| | | | J = 15.0 Hz, 1H), 4.28 (d, J = 5.8 Hz, 2H), 3.99 (d, J = 15.0 |
| | | | Hz,1H), 3.38 (s, 1H), 2.29 (d, J = 6.8 Hz, 2H), 1.97-1.66 |
| | | | (m, 2H), 1.66-1.47 (m, 2H), 1.13 (d, J = 6.4 Hz, 3H). |
| 456 & 457 | 1-(4-chloro- benzyl)-3- (4-((5- methyl-2- oxopipe ridin-1- yl)methyl) phenyl)urea | CHIRAL ART Cellulose- SB | 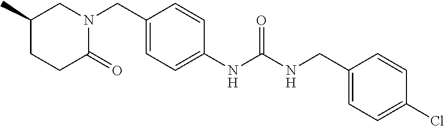 [SMILES: C[C@@H]1CCC(=O)N(Cc2ccc(NC(=O)NCc3ccc(Cl)cc3)cc2)C1] |
| | | | 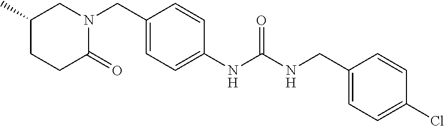 [SMILES: C[C@H]1CCC(=O)N(Cc2ccc(NC(=O)NCc3ccc(Cl)cc3)cc2)C1] |
| | | | (R)-1-(4-chlorobenzyl)-3-(4-((5-methyl-2- |
| | | | oxopiperidin-1-yl)methyl)phenyl)urea. LCMS- |
| | | | APCI (POS.) m/z: 386 (M + H)⁺. ¹H NMR (300 MHz, |
| | | | DMSO-d₆) δ 8.60 (s, 1H), 7.48-7.25 (m, 6H), 7.14- |
| | | | 7.04 (m, 2H), 6.63 (t, J = 6.0 Hz, 1H), 4.39 (s, 2H), |
| | | | 4.28 (d, J = 6.0 Hz, 2H), 3.13 (ddd, J = 11.9, 5.1, 1.8 |
| | | | Hz, 1H), 2.86-2.68 (m, 1H), 2.31 (dd, J = 8.6, 4.7 |
| | | | Hz, 2H), 1.86 (s, 1H), 1.80-1.69 (m, 1H), 1.46- |
| | | | 1.31 (m, 1H), 0.89 (d, J = 6.6 Hz, 3H). |
| | | | (S)-1-(4-chlorobenzyl)-3-(4-((5-methyl-2- |
| | | | oxopiperidin-1-yl)methyl)phenyl)urea. LCMS- |
| | | | APCI (POS.) m/z: 386 (M + H)⁺. ¹H NMR (300 MHz, |
| | | | DMSO-d₆) δ 8.58 (s, 1H), 7.48-7.25 (m, 6H), 7.14- |
| | | | 7.04 (m, 2H), 6.63 (t, J = 6.0 Hz, 1H), 4.39 (s, 2H), |
| | | | 4.28 (d, J = 6.0 Hz, 2H), 3.13 (ddd, J = 11.9, 5.1, 1.8 |
| | | | Hz, 1H), 2.86-2.68 (m, 1H), 2.31 (dd, J = 8.6, 4.7 |
| | | | Hz, 2H), 1.86 (s, 1H), 1.80-1.69 (m, 1H), 1.46- |
| | | | 1.31 (m, 1H), 0.89 (d, J = 6.6 Hz, 3H). |
| 461 & 462 | 1-(4-chloro- benzyl)-3- (4-((4- methyl- 2-oxopipe- ridin-1- yl)methyl) phenyl)urea | CHIRAL- PAK IH | 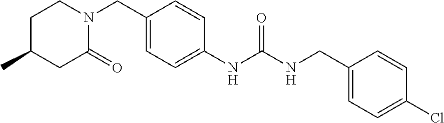 [SMILES: C[C@H]1CCN(Cc2ccc(NC(=O)NCc3ccc(Cl)cc3)cc2)C(=O)C1] |
| | | | 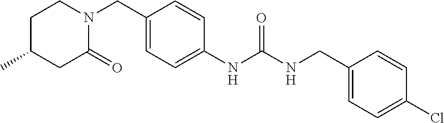 [SMILES: C[C@@H]1CCN(Cc2ccc(NC(=O)NCc3ccc(Cl)cc3)cc2)C(=O)C1] |
| | | | (S)-1-(4-chlorobenzyl)-3-(4-((4-methyl-2- |
| | | | oxopiperidin-1-yl)methyl)phenyl)urea. LCMS- |
| | | | APCI (POS.) m/z: 386 (M + H)⁺. ¹H NMR (400 MHz, |
| | | | DMSO-d₆) δ 8.60 (s, 1H), 7.39 (d, J = 8.5 Hz, 2H), |
| | | | 7.37-7.27 (m, 4H), 7.08 (d, J = 8.5 Hz, 2H), 6.64 (t, |
| | | | J = 6.1 Hz, 1H), 4.48 (d, J = 14.5 Hz, 1H), 4.37- |
| | | | 4.23 (m, 3H), 3.33 (s, 2H), 3.14 (dd, J = 8.2, 4.1 Hz, |
| | | | 2H), 2.41-2.30 (m, 1H), 1.99-1.88 (m, 1H), 1.76 |
| | | | (dt, J = 13.4, 3.0 Hz, 1H), 0.92 (d, J = 6.2 Hz, 3H). |
| | | | (R)-1-(4-chlorobenzyl)-3-(4-((4-methyl-2- |
| | | | oxopiperidin-1-yl)methyl)phenyl)urea. LCMS- |
| | | | APCI (POS.) m/z: 386 (M + H)⁺. ¹H NMR (400 MHz, |
| | | | DMSO-d₆) δ 8.59 (s, 1H), 7.39 (d, J = 8.4 Hz, 2H), |
| | | | 7.37-7.28 (m, 4H), 7.08 (d, J = 8.5 Hz, 2H), 6.64 (t, |
| | | | J = 6.0 Hz, 1H), 4.48 (d, J = 14.5 Hz, 1H), 4.36- |
| | | | 4.22 (m, 3H), 3.33 (s, 2H), 3.14 (dd, J = 8.1, 4.1 Hz, |
| | | | 2H), 2.36 (dt, J = 14.3, 2.4 Hz, 1H), 1.99-1.89 (m, |
| | | | 1H), 1.76 (dt, J = 13.3, 3.0 Hz, 1H), 0.92 (d, J = 6.1 |
| | | | Hz, 3H). |
| 463 | 1-(4- methoxy benzyl)- 3-(4-((4- methyl- 2-oxopipe- ridin-1- yl)methyl) | CHIRAL- PAK AD-H | 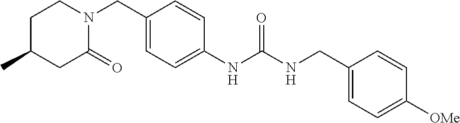 [SMILES: COc1ccc(CNC(=O)Nc2ccc(CN3CC[C@H](C)CC3=O)cc2)cc1] |
| | phenyl)urea | | |
| | | | 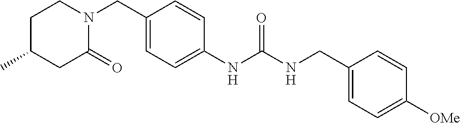 [SMILES: COc1ccc(CNC(=O)Nc2ccc(CN3CC[C@@H](C)CC3=O)cc2)cc1] |
| | | | (S)-1-(4-methoxybenzyl)-3-(4-((4-methyl-2- |
| | | | oxopiperidin-1-yl)methyl)phenyl)urea. . LCMS- |
| | | | APCI (POS.) m/z: 382 (M + H)⁺. ¹H NMR (300 MHz, |
| | | | DMSO-d₆) δ 8.48 (s, 1H), 7.34 (d, J = 8.4 Hz, 2H), |
| | | | 7.22 (d, J = 8.6 Hz, 2H), 7.08 (d, J = 8.4 Hz, 2H), |
| | | | 6.94-6.85 (m, 2H), 6.49 (t, J = 5.9 Hz, 1H), 4.49 (d, |
| | | | J = 14.5 Hz, 1H), 4.32 (d, J = 14.5 Hz, 1H), 4.21 (d, |
| | | | J = 5.8 Hz, 2H), 3.73 (s, 3H), 3.15 (dd, J = 8.1, 4.1 Hz, |
| | | | 2H), 2.36 (d, J = 13.6 Hz, 1H), 1.96 (d, J = 10.3 Hz, |
| | | | 1H), 1.77 (d, J = 13.1 Hz, 2H), 1.35 (s, 1H), 0.93 (d, |
| | | | J = 6.0 Hz, 3H). |
| | | | (R)-1-(4-methoxybenzyl)-3-(4-((4-methyl-2- |
| | | | oxopiperidin-1-yl)methyl)phenyl)urea. . LCMS- |
| | | | APCI (POS.) m/z: 382 (M + H)⁺. ¹H NMR (300 MHz, |
| | | | DMSO-d₆) δ 8.48 (s, 1H), 7.34 (d, J = 8.4 Hz, 2H), |
| | | | 7.27-7.17 (m, 2H), 7.08 (d, J = 8.4 Hz, 2H), 6.94- |
| | | | 6.85 (m, 2H), 6.49 (t, J = 5.9 Hz, 1H), 4.49 (d, J = |
| | | | 14.5 Hz, 1H), 4.32 (d, J = 14.5 Hz, 1H), 4.21 (d, J = |
| | | | 5.8 Hz, 2H), 3.73 (s, 3H), 3.15 (dd, J = 8.1, 4.1 Hz, |
| | | | 2H), 2.34 (s, 1H), 1.96 (d, J = 10.3 Hz, 1H), 1.77 (d, |
| | | | J = 13.2 Hz, 2H), 1.35 (s, 1H), 1.24 (s, 1H), 0.93 (d, J = |
| | | | 6.0 Hz, 3H). |
| 467 & 468 | 1-(4-chloro- benzyl)-3- (4-((3- methyl-2- oxopipe- ridin-1- yl)methyl) phenyl)urea | CHIRAL ART Cellulose- SC | 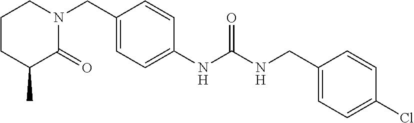 [SMILES: C[C@H]1CCCN(Cc2ccc(NC(=O)NCc3ccc(Cl)cc3)cc2)C1=O] |
| | | | 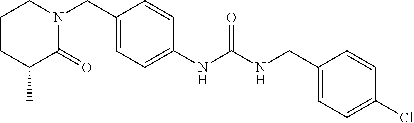 [SMILES: C[C@@H]1CCCN(Cc2ccc(NC(=O)NCc3ccc(Cl)cc3)cc2)C1=O] |
| | | | (S)-1-(4-chlorobenzyl)-3-(4-((3-methyl-2- |
| | | | oxopiperidin-1-yl)methyl)phenyl)urea. LCMS- |
| | | | APCI (POS.) m/z: 386 (M + H)⁺. ¹H NMR (300 MHz, |
| | | | DMSO-d₆) δ 8.57 (s, 1H), 7.42-7.21 (m, 6H), 7.08 (d, |
| | | | 8.2 Hz, 2H), 6.63 (t, J = 6.0 Hz, 1H), 4.50-4.32 (m, |
| | | | 2H), 4.28 (d, J = 5.9 Hz, 2H), 3.16 (dd, J = 7.2, 5.0 Hz, |
| | | | 2H), 2.34 (q, J = 7.2 Hz, 1H), 1.94-1.81 (m, 1H), 1.79- |
| | | | 1.61 (m, 2H), 1.51-1.36 (m, 1H), 1.14 (d, J = 7.1 Hz, 3H). |
| | | | (R)-1-(4-chlorobenzyl)-3-(4-((3-methyl-2- |
| | | | oxopiperidin-1-yl)methyl)phenyl)urea. LCMS- |
| | | | APCI (POS.) m/z: 386 (M + H)⁺. ¹H NMR (300 MHz, |
| | | | DMSO-d₆) δ 8.56 (s, 1H), 7.41-7.17 (m, 6H), 7.08 (d, |
| | | | J = 8.2 Hz, 2H), 6.63 (t, J = 6.0 Hz, 1H), 4.51-4.36 (m, |
| | | | 2H), 4.32-4.20 (m, 2H), 3.16 (t, J = 6.0 Hz, 2H), 2.39- |
| | | | 2.29 (m, 1H), 1.89 (ddd, J = 12.2, 9.3, 5.6 Hz, 1H), 1.79- |
| | | | 1.61 (m, 2H), 1.50-1.36 (m, 1H), 1.14 (d, J = 7.1 Hz, 3H). |
| 469 & 470 | 1-(4- methoxy- benzyl)- 3-(4-((3- methyl-2- oxopipe- ridin-1- yl)methyl) | CHIRAL ART Cellulose- SB | 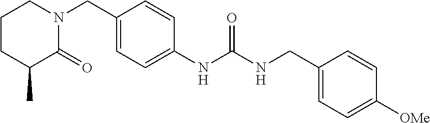 [SMILES: COc1ccc(CNC(=O)Nc2ccc(CN3CCC[C@H](C)C3=O)cc2)cc1] |
| | phenyl)urea. | | |
| | | | 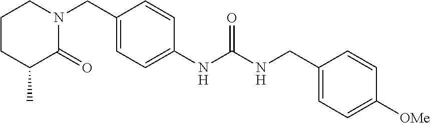 [SMILES: COc1ccc(CNC(=O)Nc2ccc(CN3CCC[C@@H](C)C3=O)cc2)cc1] |
| | | | (S)-1-(4-methoxybenzyl)-3-(4-((3-methyl-2- |
| | | | oxopiperidin-1-yl)methyl)phenyl)urea. LCMS- |
| | | | APCI (POS.) m/z: 382 (M + H)⁺. ¹H NMR (300 MHz, |
| | | | DMSO-d₆) δ 8.47 (d, J = 4.9 Hz, 1H), 7.36 (d, J = 8.2 Hz, |
| | | | 2H), 7.24 (d, J = 8.1 Hz, 2H), 7.09 (d, J = 8.0 Hz, 2H), |
| | | | 6.94-6.83 (m, 2H), 6.49 (q, J = 7.2, 6.4 Hz, 1H), 4.38 (td, |
| | | | J = 14.5, 5.0 Hz, 2H), 4.22 (t, J = 5.8 Hz, 2H), 3.74 (d, J = |
| | | | 5.2 Hz, 3H), 3.15 (t, J = 5.8 Hz, 2H), 1.89-1.65 (m, 3H), |
| | | | 1.50-1.35 (m, 2H), 1.15 (t, J = 6.5 Hz, 3H). |
| | | | (R)-1-(4-methoxybenzyl)-3-(4-((3-methyl-2- |
| | | | oxopiperidin-1-yl)methyl)phenyl)urea. LCMS- |
| | | | APCI (POS.) m/z: 382 (M + H)⁺. ¹H NMR (300 MHz, |
| | | | DMSO-d₆) δ 8.46 (s, 1H), 7.38-7.28 (m, 2H), 7.28-7.17 |
| | | | (m, 2H), 7.11-7.03 (m, 2H), 6.94-6.85 (m, 2H), 6.48 (t, |
| | | | J = 5.8 Hz, 1H), 4.37 (t, J = 14.6 Hz, 2H), 4.22 (d, J = 5.8 |
| | | | Hz, 2H), 3.74 (s, 3H), 3.16 (dd, J = 7.2, 5.0 Hz, 2H), 2.36- |
| | | | 2.29 (m, 1H), 1.90 (ddd, J = 12.8, 6.2, 3.4 Hz, 1H), 1.77- |
| | | | 1.64 (m, 2H), 1.48-1.37 (m, 1H), 1.14 (d, J = 7.2 Hz, 3H). |
| 542 & 543 | 1-(4-(1,1- dioxido- thiomor- pholin-3-yl) phenyl)-3- (4-methoxy- benzyl)urea. (Interme- diate 32) | CHIRAL- PAK IA | 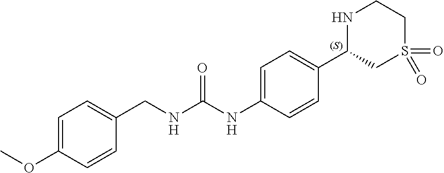 |
| | | | 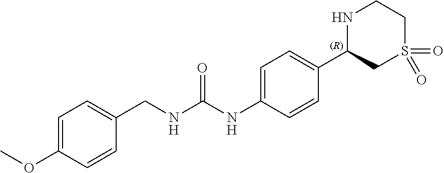 |
| | | | (S)-1-(4-(1,1-dioxidothiomorpholin-3-yl)phenyl)-3- |
| | | | (4-methoxybenzyl)urea. LCMS-APCI (POS.) m/z: |
| | | | 390 (M + H)⁺. ¹H NMR (300 MHz, DMSO-d₆) δ 8.51 |
| | | | (s, 1H), 7.40-7.32 (m, 2H), 7.29-7.18 (m, 4H), 6.94- |
| | | | 6.85 (m, 2H), 6.52 (t, J = 5.9 Hz, 1H), 4.21 (d, J = 5.8 Hz, |
| | | | 2H), 3.98-3.89 (m, 1H), 3.73 (s, 3H), 3.15 (dt, J = 13.4, |
| | | | 3.0 Hz, 2H), 3.11-2.98 (m, 4H), 2.73 (s, 1H). |
| | | | (R)-1-(4-(1,1-dioxidothiomorpholin-3-yl)phenyl)-3- |
| | | | (4-methoxybenzyl)urea. LCMS-APCI (POS.) m/z: |
| | | | 390 (M + H)⁺. ¹H NMR (300 MHz, DMSO-d₆) δ 8.51 |
| | | | (s, 1H), 7.40-7.33 (m, 2H), 7.29-7.18 (m, 4H), 6.94- |
| | | | 6.85 (m, 2H), 6.52 (t, J = 5.9 Hz, 1H), 4.21 (d, J = 5.8 Hz, |
| | | | 2H), 3.98-3.89 (m, 1H), 3.73 (s, 3H), 3.15 (dt, J = 13.4, |
| | | | 2.9 Hz, 2H), 3.11-2.98 (m, 4H), 2.73 (s, 1H). |
| 544 & 545 | 1-(4-(1,1- dioxido- thiomor- pholin-2-yl) phenyl)-3- (4-methoxy benzyl)urea (Interme- diate 33): | CHIRAL ART Cellulose- SB | 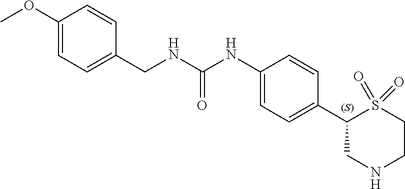 |
| | | | 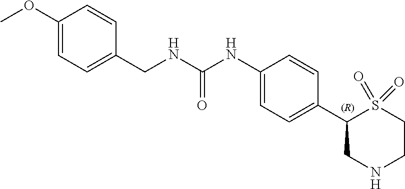 |
| | | | (S)-1-(4-(1,1-dioxidothiomorpholin-2-yl)phenyl)-3- |
| | | | (4-methoxybenzyl)urea. LCMS-APCI (POS.) m/z: |
| | | | 390 (M + H)⁺. ¹H NMR (300 MHz, DMSO-d₆) δ 8.63 |
| | | | (s, 1H), 7.41 (d, J = 8.3 Hz, 2H), 7.23 (dd, J = 8.4, 5.5 |
| | | | Hz, 4H), 6.96-6.86 (m, 2H), 6.57 (t, J = 5.9 Hz, 1H), |
| | | | 4.23 (d, J = 5.7 Hz, 2H), 4.14 (t, J = 7.4 Hz, 1H), 3.74 |
| | | | (s, 3H), 3.36 (s, 1H), 3.27-2.96 (m, 4H), 2.65 (s, 2H). |
| | | | (R)-1-(4-(1,1-dioxidothiomorpholin-2-yl)phenyl)-3- |
| | | | (4-methoxybenzyl)urea. LCMS-APCI (POS.) m/z: 390 |
| | | | (M + H)⁺. ¹H NMR (300 MHz, DMSO-d₆) δ 8.68 (s, 1H), |
| | | | 7.41 (d, J = 8.3 Hz, 2H), 7.28-7.17 (m, 4H), 6.96-6.86 |
| | | | (m, 2H), 6.63 (t, J = 6.0 Hz, 1H), 4.23 (d, J = 5.8 Hz, |
| | | | 2H), 4.13 (t, J = 7.4 Hz, 1H), 3.74 (s, 3H), 3.30-3.19 |
| | | | (m, 3H), 3.18-3.02 (d, J = 6.9Hz, 2H), 2.60 (s, 2H). |
| 538 & 539 | 1-(4-chloro- benzyl)-3- (4-(1- (methyl- sulfonyl) pyrrolidin- 3-yl) phenyl)urea (Interme- diate 35): | CHIRAL- PAK ID | 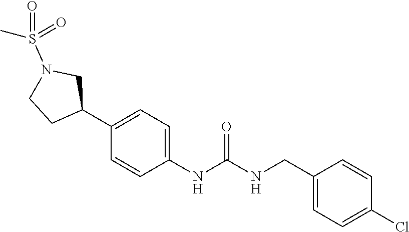 [SMILES: CS(=O)(=O)N1CC[C@H](c2ccc(NC(=O)NCc3ccc(Cl)cc3)cc2)C1] |
| | | | 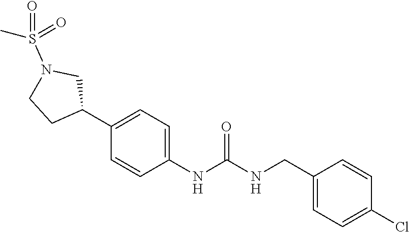 [SMILES: CS(=O)(=O)N1CC[C@@H](c2ccc(NC(=O)NCc3ccc(Cl)cc3)cc2)C1] |
| | | | (R)-1-(4-chlorobenzyl)-3-(4-(1- |
| | | | (methylsulfonyl)pyrrolidin-3-yl)phenyl)urea. |
| | | | LCMS-APCI (POS.) m/z: 408 (M + H)⁺. ¹H NMR |
| | | | (300 MHz, DMSO-d₆) δ 8.56 (s, 1H), 7.43-7.29 (m, |
| | | | 6H), 7.22-7.15 (m, 2H), 6.63 (t, J = 6.1 Hz, 1H), 4.28 (d, |
| | | | J = 6.0 Hz, 2H), 3.69-3.61 (m, 1H), 3.45 (t, J = 9.4 Hz, |
| | | | 1H), 3.32 (t, J = 6.1 Hz, 2H), 3.32 (s, 1H), 3.09 (t, J = 9.6 |
| | | | Hz, 1H), 2.95 (s, 2H), 2.27-2.19 (m, 1H), 1.96 (q, J = |
| | | | 10.2 Hz, 1H). |
| | | | (S)-1-(4-chlorobenzyl)-3-(4-(1- |
| | | | (methylsulfonyl)pyrrolidin-3-yl)phenyl)urea. |
| | | | LCMS-APCI (POS) m/z: 408 (M + H)⁺. ¹H NMR |
| | | | (300 MHz, DMSO-d₆) δ 8.57 (s, 1H), 7.43-7.36 (m, |
| | | | 2H), 7.39-7.29 (m, 4H), 7.18 (d, J = 8.5 Hz, 2H), 6.63 (t, |
| | | | J = 6.0 Hz, 1H), 4.28 (d, J = 5.9 Hz, 2H), 3.65 (dd, J = 9.6, |
| | | | 7.5 Hz, 1H), 3.46 (ddd, J = 10.6, 8.4, 2.5 Hz, 1H), 3.32 (td, |
| | | | J = 9.7, 6.6 Hz, 2H), 3.10 (t, J = 9.6 Hz, 1H), 2.95 (s, 3H), |
| | | | 2.28-2.18 (m, 1H), 1.95 (p, J = 10.0 Hz, 1H). |
| 540 & 541 | 1-(4-metho- xybenzyl)- 3-(4-(1- (methyl- sulfonyl) pyrrolidin- 3-yl) phenyl)urea. | CHIRAL- PAK IA | 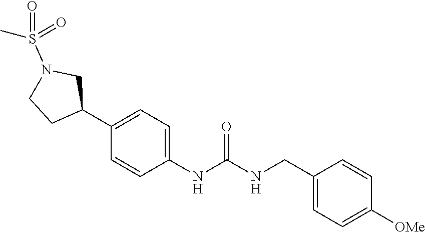 [SMILES: COc1ccc(CNC(=O)Nc2ccc([C@H]3CCN(S(C)(=O)=O)C3)cc2)cc1] |
| | | | 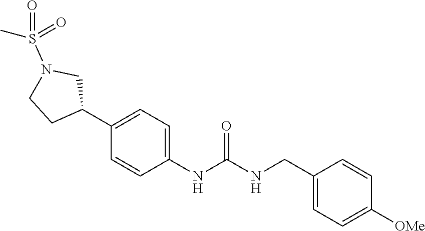 [SMILES: COc1ccc(CNC(=O)Nc2ccc([C@@H]3CCN(S(C)(=O)=O)C3)cc2)cc1] |
| | | | (R)-1-(4-methoxybenzyl)-3-(4-(1- |
| | | | (methylsulfonyl)pyrrolidin-3-yl)phenyl)urea. LC/MS |
| | | | (APCI) m/z: 404 [M + H]. ¹H NMR (300 MHz, DMSO- |
| | | | d₆) δ 8.47 (s, 1H), 7.39-7.32 (m, 2H), 7.27-7.15 (m, |
| | | | 4H), 6.94-6.85 (m, 2H), 6.49 (t, J = 5.9 Hz, 1H), 4.22 (d, |
| | | | J = 5.8 Hz, 2H), 3.73 (s, 3H), 3.65 (dd, J = 9.6, 7.5 Hz, |
| | | | 1H), 3.46 (ddd, J = 10.5, 8.4, 2.5 Hz, 1H), 3.32 (td, J = 9.8, |
| | | | 6.7 Hz, 2H), 3.09 (t, J = 9.6 Hz, 1H), 2.95 (s, 3H), 2.23 |
| | | | (dtd, J = 12.9, 6.6, 2.4 Hz, 1H), 1.94 (ddd, J = 20.6, 12.2, |
| | | | 9.9 Hz, 1H). |
| | | | (S)-1-(4-methoxybenzyl)-3-(4-(1- |
| | | | (methylsulfonyl)pyrrolidin-3-yl)phenyl)urea. LC/MS |
| | | | (APCI) m/z: 404 [M + H]. ¹H NMR (300 MHz, DMSO- |
| | | | d₆) δ 8.48 (d, J = 3.4 Hz, 1H), 7.35 (d, J = 8.3 Hz, 2H), |
| | | | 7.23 (d, J = 8.2 Hz, 2H), 7.18 (d, J = 8.1 Hz, 2H), 6.90 (d, |
| | | | J = 8.2 Hz, 2H), 6.50 (q, J = 5.4 Hz, 1H), 4.22 (d, J = 5.8 |
| | | | Hz, 2H), 3.73 (s, 3H), 3.65 (dd, J = 9.6, 7.5 Hz, 1H), 3.50- |
| | | | 3.41 (m, 1H), 3.33 (td, J = 6.8, 4.0 Hz, 2H), 3.09 (t, J = |
| | | | 9.6 Hz, 1H), 2.95 (s, 3H), 2.26-2.17 (m, 1H), 1.94 (p, J = |
| | | | 10.1 Hz, 1H). |

### Example 12

### Synthesis of 1-(4-methoxybenzyl)-3-(4-((4-methyl-2-oxopiperazin-1-yl)methyl)phenyl)urea. (Compound 384)

### Step 1: Preparation of 1-(4-methoxybenzyl)-3-(4-((2-oxopiperazin-1-yl)methyl)phenyl)urea (Intermediate 3-a)

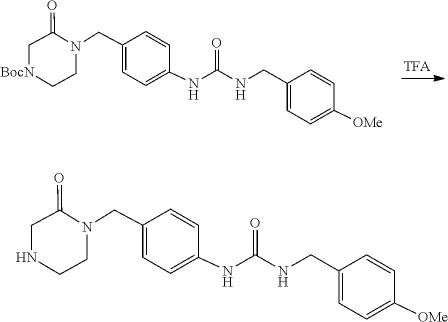 [SMILES: COc1ccc(CNC(=O)Nc2ccc(CN3CCN(C(=O)OC(C)(C)C)CC3=O)cc2)cc1 ; COc1ccc(CNC(=O)Nc2ccc(CN3CCNCC3=O)cc2)cc1]

To a stirred solution of tert-butyl 4-(4-(3-(4-methoxybenzyl)ureido)benzyl)-3-oxopiperazine-1-carboxylate (376 mg, 0.802 mmol, 1 equiv) in DCM was added TFA (1 mL). The resulting mixture was stirred at r.t. for 1 h, concentrated under reduced pressure, and purified by C18 column chromatography, eluted with water(0.05% NH₄HCO₃)/ACN (2:1) to give a crude product, which was purified by Prep-HPLC with the following conditions (2#SHIMADZU (HPLC-01)): Column, XBridge Prep OBD C₁₈ Column, 30×150 mm 5 um; mobile phase, Water(10 MMOL/L NH4HCO3) and ACN (30% Phase B up to 60% in 8 min); Detector, uv254 nm to afford 60 mg of 1-(4-methoxybenzyl)-3-(4-((2-oxopiperazin-1-yl)methyl)phenyl)urea (20.3 mg, 20%) as an off-white solid. LCMS-APCI (POS.) m/z: 369 (M+H)+.

### Step 2: Preparation of 1-(4-methoxybenzyl)-3-(4-((4-methyl-2-oxopiperazin-1-yl)methyl)phenyl)urea (Intermediate 3-a)

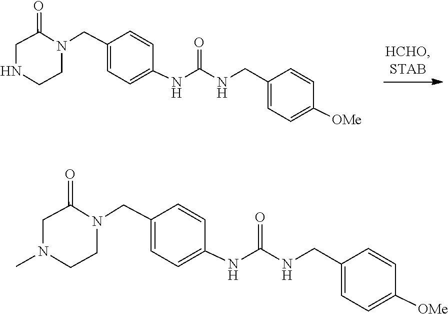 [SMILES: COc1ccc(CNC(=O)Nc2ccc(CN3CCNCC3=O)cc2)cc1 ; COc1ccc(CNC(=O)Nc2ccc(CN3CCN(C)CC3=O)cc2)cc1]

To a solution of 3-[(4-methoxyphenyl)methyl]-1-[4-[(2-oxopiperazin-1-yl)methyl]phenyl]urea (35.00 mg, 0.095 mmol, 1.00 equiv) in DCE (3 mL) was added formaldehyde (68.40 mg, 0.760 mmol, 8.00 equiv). After stirred at r.t. for 10 min, the mixture was added STAB (80.53 mg, 0.380 mmol, 4 equiv) and AcOH (22.82 mg, 0.380 mmol, 4 equiv). The resulting mixture was stirred at r.t. for 3 h. Water (20 mL) was added and the mixture was extracted twice with EtOAc (20 mL). The combined organic layers were washed twice with brine (20 mL), dried over anhydrous Na₂SO₄, concentrated under reduced pressure, and purified by Prep-HPLC with the following conditions (2#SHIMADZU (HPLC-01)): Column, XBridge Prep OBD C18 Column, 30*150 mm 5 um; mobile phase, Water (10 mmol/L NH₄HCO₃) and ACN (18% Phase B up to 36% in 8 min); Detector, UV254 nm to afford 6.3 mg of 1-(4-methoxybenzyl)-3-(4-((4-methyl-2-oxopiperazin-1-yl)methyl)phenyl)urea (17%) as a white solid. LCMS-APCI (POS.) m/z: 383 (M+H)+. ¹H NMR (400 MHz, Methanol-d₄) δ 7.36 (d, J=8.5 Hz, 2H), 7.26 (d, J=8.7 Hz, 2H), 7.20 (d, J=8.5 Hz, 2H), 6.90 (d, J=8.7 Hz, 2H), 4.55 (s, 2H), 4.32 (s, 2H), 3.79 (s, 3H), 3.32-3.28 (m, 2H), 3.17 (s, 2H), 2.72-2.62 (m, 2H), 2.34 (s, 3H).

### Example 13

### Synthesis of 1-(4-((8-oxa-3-azabicyclo[3.2.1]octan-3-yl)sulfonyl)phenyl)-3-(4-chlorobenzyl)urea. (Compound 526)

### Preparation of 1-(4-((8-oxa-3-azabicyclo[3.2.1]octan-3-yl)sulfonyl)phenyl)-3-(4-chlorobenzyl)urea

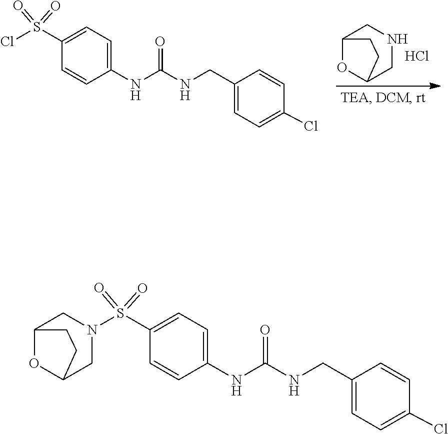 [SMILES: O=C(NCc1ccc(Cl)cc1)Nc1ccc(S(=O)(=O)Cl)cc1 ; C1CC2CNCC1O2 ; Cl ; O=C(NCc1ccc(Cl)cc1)Nc1ccc(S(=O)(=O)N2CC3CCC(C2)O3)cc1]

To a stirred mixture of 4-({[(4-chlorophenyl)methyl]carbamoyl}amino)benzenesulfonyl chloride (100 mg, 0.278 mmol, 1.00 equiv) and TEA (84.6 mg, 0.836 mmol, 3.00 equiv) in DCM (1 mL) was added 8-oxa-3-azabicyclo[3.2.1]octane hydrochloride (41.6 mg, 0.278 mmol, 1.00 equiv). The resulting mixture was stirred at r.t. for 2 h, concentrated under reduced pressure, purified by Prep-HPLC with the following conditions (Column, XBridge Prep OBD C18 Column, 30*150 mm, 5 μm; mobile phase, water(10 mmol/L NH₄HCO₃+0.1% NH₃. H₂O) and ACN (25% ACN up to 55% in 8 m)) to afford 20.8 mg of 1-(4-((8-oxa-3-azabicyclo[3.2.1]octan-3-yl)sulfonyl)phenyl)-3-(4-chlorobenzyl)urea (17.14%) as a white solid. LCMS-APCI (POS.) m/z: 436 (M+H)+. ¹H NMR (300 MHz, DMSO-d₆) δ 9.17 (s, 11H), 7.77-7.51 (m, 4H), 7.46-7.14 (m, 4H), 6.87 (t, =6.0 Hz, 1H), 4.32 (t, J=5.0 Hz, 4H), 3.22 (d, J=11.1 Hz, 2H), 2.54 (s, 2H), 1.93-1.56 (m, 4H).

Compounds in the following table were prepared in a similar manner as Compound 526, using the intermediates and reagents as listed.

| Ex | | | |
|---|---|---|---|
| # | Intermediate | Amine | Structure, Name and Data |
| 527 | Intermediate 36 | pyrrolidine | 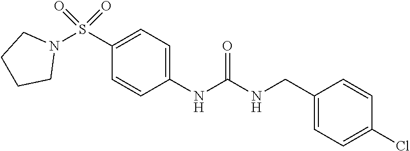 [SMILES: O=C(NCc1ccc(Cl)cc1)Nc1ccc(S(=O)(=O)N2CCCC2)cc1] |
| | | | 1-(4-chlorobenzyl)-3-(4-(pyrrolidin-1- |
| | | | ylsulfonyl)phenyl)urea.LCMS-APCI |
| | | | (POS.) m/z: 394 (M + H)+. ¹H NMR (300 |
| | | | MHz, DMSO-d₆) δ 9.13 (s, 1H), 7.71-7.57 |
| | | | (m, 4H), 7.45-7.28 (m, 4H), 6.86 (t, J = 6.1 |
| | | | Hz, 1H), 4.31 (d, J = 5.9 Hz, 2H), 3.15-3.04 |
| | | | (m, 4H), 1.63 (p, J = 3.6, 2.7 Hz, 4H). |
| 528 | Intermediate 36 | 3- methylaze- tidine-3- carbonitrile hydro- chloride | 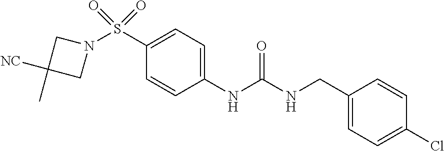 [SMILES: CC1(C#N)CN(S(=O)(=O)c2ccc(NC(=O)NCc3ccc(Cl)cc3)cc2)C1] |
| | | | 1-(4-chlorobenzyl)-3-(4-((3-cyano-3- |
| | | | methylazetidin-1-yl)sulfonyl)phenyl)urea. |
| | | | LCMS-APCI (POS.) m/z: 419 (M + H)+. ¹H |
| | | | NMR (300 MHz, DMSO-d₆) δ 9.26 (s, 1H), |
| | | | 7.72 (s, 4H), 7.53-7.21 (m, 4H), 6.92 (t, J = |
| | | | 6.0 Hz, 1H), 4.32 (d, J = 5.9 Hz, 2H), 3.97 |
| | | | (d, J = 8.8 Hz, 2H), 3.65 (d, J = 8.8 Hz, 2H), |
| | | | 1.36 (s, 3H). |
| 529 | Intermediate 36 | 3- pyridine- methanea- mine | 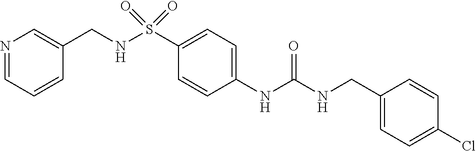 [SMILES: O=C(NCc1ccc(Cl)cc1)Nc1ccc(S(=O)(=O)NCc2cccnc2)cc1] |
| | | | 4-(3-(4-chlorobenzyl)ureido)-N-(pyridin- |
| | | | 3-ylmethyl)benzenesulfonamide. LCMS- |
| | | | APCI (POS.) m/z: 431 (M + H)+ ¹H NMR |
| | | | (300 MHz, DMSO-d₆) δ 9.06 (s, 1H), 8.43 |
| | | | (d, J = 5.5 Hz, 2H), 7.98 (s, 1H), 7.75-7.51 |
| | | | (m, 5H), 7.46-7.22 (m, 5H), 6.84 (t, J = 6.0 |
| | | | Hz, 1H), 4.31 (d, J = 5.9 Hz, 2H), 3.99 (d, |
| | | | J = 4.9 Hz, 2H). |

### Example 14

### Synthesis of 1-(4-chlorobenzyl)-3-(4-(2-(pyridin-4-yloxy)ethyl)phenyl)urea. (Compound 485)

### Preparation of 1-(4-chlorobenzyl)-3-(4-(2-(pyridin-4-yloxy)ethyl)phenyl)urea

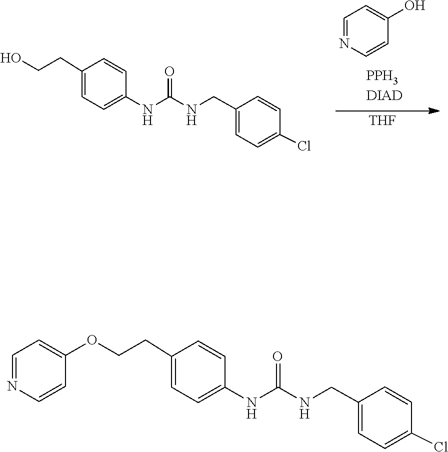 [SMILES: Oc1ccncc1 ; O=C(NCc1ccc(Cl)cc1)Nc1ccc(CCO)cc1 ; O=C(NCc1ccc(Cl)cc1)Nc1ccc(CCOc2ccncc2)cc1 ; "THF"]

To a solution of 1-(4-chlorobenzyl)-3-(4-(2-hydroxyethyl)phenyl)urea (Intermediate 38, 65 mg, 0.213 mmol, 1.0 equiv) in THE (1 mL) was added PPh₃ (112 mg, 0.427 mmol, 2.0 equiv), pyridin-4-ol (41 mg, 0.427 mmol, 2.0 equiv) and diisopropyl azodicarboxylate (86 mg, 0.427 mmol, 2.0 equiv) sequentially. The reaction was stirred at 23° C. for 24 h. LC-MS showed generally half conversion. Then the reaction was concentrated and purified by preparative HPLC (H₂O (0.1% HCO₂H)/MeCN (0.1% HCO₂H) to yield 1-(4-chlorobenzyl)-3-(4-(2-(pyridin-4-yloxy)ethyl)phenyl)urea (7 mg, 9). LCMS-ESI (Pa t.) m/z: 382.10 (M+H)⁺. ¹H NM/R (400 MHz, DMSO-d₆) δ 8.53 (s, 1H), 8.36 (d, J=5.5 Hz, 2H), 7.39 (d, J=8.5 Hz, 2H), 7.36-7.29 (m, 4H), 7.17 (d, J=8.4 Hz, 2H), 6.96 (d, J=5.6 Hz, 2H), 6.61 (t, J=6.0 Hz, 1H), 4.28 (d, J=6.0 Hz, 2H), 4.23 (t, J=6.9 Hz, 2H), 2.96 (t, J=6.9 Hz, 2H).

Compounds in the following table were prepared in a similar manner as Compound 485, using the intermediates and reagents as listed.

| Ex | | | |
|---|---|---|---|
| # | Intermediate | Phenol | Structure, Name and Data |
| 486 | Intermediate 38 | pyridin-3- ol | 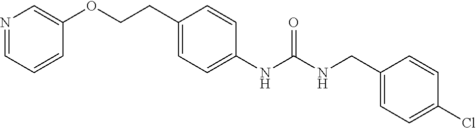 [SMILES: O=C(NCc1ccc(Cl)cc1)Nc1ccc(CCOc2cccnc2)cc1] |
| | | | 1-(4-chlorobenzyl)-3-(4-(2-(pyridin-3- |
| | | | yloxy)ethyl)phenyl)urea. LCMS-ESI (POS.) |
| | | | m/z: 382.10 (M + H)⁺. ¹H NMR (400 MHz, |
| | | | DMSO-d₆) δ 8.53 (s, 1H), 8.28 (s, 1H), 8.16 |
| | | | (d, J = 4.4 Hz, 1H), 7.43-7.36 (m, 3H), 7.36- |
| | | | 7.28 (m, 5H), 7.18 (d, J = 8.5 Hz, 2H), 6.62 (t, |
| | | | J = 6.0 Hz, 1H), 4.28 (d, J = 5.9 Hz, 2H), 4.21 |
| | | | (t, J = 6.9 Hz, 2H), 2.96 (t, J = 6.9 Hz, 2H). |
| 494 | Intermediate 3.2-b | pyridin-3- ol | 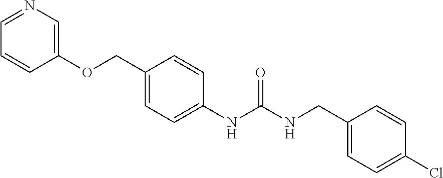 [SMILES: O=C(NCc1ccc(Cl)cc1)Nc1ccc(COc2cccnc2)cc1] |
| | | | 1-(4-chlorobenzyl)-3-(4-((pyridin-3- |
| | | | yloxy)methyl)phenyl)urea. LCMS-ESI (POS.) |
| | | | m/z: 367.90 (M + H)⁺. ¹H NMR (400 MHz, |
| | | | DMSO-d₆) δ 8.77 (s, 1H), 7.52-7.41 (m, 4H), |
| | | | 7.39 (d, J = 8.4 Hz, 2H), 7.35 (d, J = 8.7 Hz, |
| | | | 2H), 7.31 (d, J = 8.5 Hz, 2H), 7.23 (dd, J = 9.0, |
| | | | 5.6 Hz, 1H), 6.85 (dd, J = 8.7, 2.5 Hz, 1H), |
| | | | 6.72 (t, J = 5.7 Hz, 1H), 4.27 (d, J = 6.0 Hz, |
| | | | 2H), 3.17 (s, 2H). |

### Example 15

### Synthesis of (R)-4-(3-(4-methoxybenzyl)ureido)-N-(1-(3-methylpyridin-2-yl)ethyl)benzamide. (Compound 513)

### Preparation of (R)-4-(3-(4-methoxybenzyl)ureido)-N-(1-(3-methylpyridin-2-yl)ethyl)benzamide

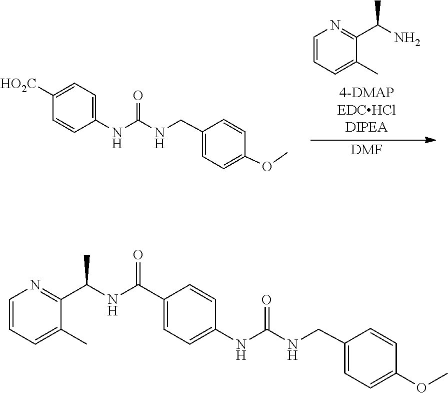

To a vial charged with 4-(3-(4-methoxybenzyl)ureido)benzoic acid (Intermediate 1.4, 60 mg, 0.200 mmol, 1.0 equiv), (R)-1-(3-methylpyridin-2-yl)ethan-1-amine (33 mg, 0.240 mmol, 1.2 equiv), 3-(ethyliminomethyleneamino)-N,N-dimethyl-propan-1-amine (EDC) HCl salt (46 mg, 0.240 mmol, 1.2 equiv), and 4-DMAP (12 mg, 0.100 mmol, 0.5 equiv) was added DMF (1 mL) and diisopropylethylamine (76 mg, 0.600 mmol, 3.0 equiv). The reaction was stirred at 23° C. for 24 h. Then the crude mixture was directly subjected to preparative HPLC (H₂O (0.1% HCO₂H)/MeCN (0.1% HCO₂H) to yield (R)-4-(3-(4-methoxybenzyl)ureido)-N-(1-(3-methylpyridin-2-yl)ethyl)benzamide (22 mg, 22%) as a white solid.

Compounds in the following table were prepared in a similar manner as Compound 513, using the intermediates and reagents as listed.

| Ex | | |
|---|---|---|
| # | Amine | Structure, Name and Data |
| 501 | 8-oxa-3- azabicyclo [3.2.1]oc- tane | 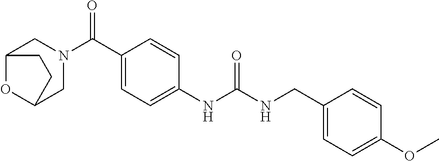 [SMILES: COc1ccc(CNC(=O)Nc2ccc(C(=O)N3CC4CCC(C3)O4)cc2)cc1] |
| | | 1-(4-(8-oxa-3-azabicyclo[3.2.1]octane-3- |
| | | carbonyl)phenyl)-3-(4-methoxybenzyl)urea. LCMS- |
| | | ESI (POS.) m/z: 396.15 (M + H)⁺. ¹H NMR (400 MHz, |
| | | DMSO-d₆) δ 8.73 (s, 1H), 7.45 (d, J = 8.6 Hz, 2H), |
| | | 7.27 (d, J = 8.6 Hz, 2H), 7.23 (d, J = 8.6 Hz, 2H), |
| | | 6.90 (d, J = 8.6 Hz, 2H), 6.60 (t, J = 5.9 Hz, 1H), |
| | | 4.37-4.22 (m, 2H), 4.23 (d, J = 5.8 Hz, 2H), 3.73 (s, |
| | | 3H), 3.38-3.27 (m, 2H), 2.55 (s, 2H), 1.86-1.71 (m, |
| | | 2H), 1.71-1.51 (m, 2H). |
| 512 | (R)-1- (pyridin- 2- yl)ethan- 1-amine | 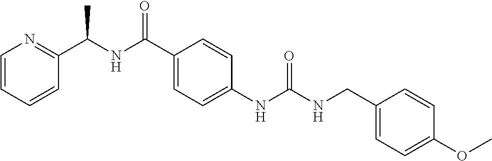 [SMILES: COc1ccc(CNC(=O)Nc2ccc(C(=O)N[C@H](C)c3ccccn3)cc2)cc1] |
| | | (R)-4-(3-(4-methoxybenzyl)ureido)-N-(1-(pyridin-2- |
| | | yl)ethyl)benzamide. LCMS-ESI (POS.) m/z: 405.1 |
| | | (M + H)⁺. ¹H NMR (400 MHz, DMSO-d₆) δ 8.80 (s, |
| | | 1H), 8.61 (d, J = 7.8 Hz, 1H), 8.52 (dd, J = 4.9, 1.6 Hz, |
| | | 1H), 7.82 (d, J = 8.7 Hz, 2H), 7.75 (td, J = 7.7, 1.8 |
| | | Hz, 1H), 7.47 (d, J = 8.8 Hz, 2H), 7.39 (d, J = 7.9 Hz, |
| | | 1H), 7.28-7.20 (m, 3H), 6.90 (d, J = 8.6 Hz, 2H), |
| | | 6.64 (t, J = 5.9 Hz, 1H), 5.17 (p, J = 7.1 Hz, 1H), 4.23 |
| | | (d, J = 5.8 Hz, 2H), 3.73 (s, 3H), 1.49 (d, J = 7.1 Hz, |
| | | 3H). |
| 513 | (R)-1-(3- methyl- pyridin-2- yl)ethan- 1-amine | 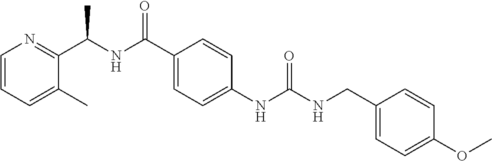 [SMILES: COc1ccc(CNC(=O)Nc2ccc(C(=O)N[C@H](C)c3ncccc3C)cc2)cc1] |
| | | (R)-4-(3-(4-methoxybenzyl)ureido)-N-(1-(3- |
| | | methylpyridin-2-yl)ethyl)benzamide. LCMS-ESI |
| | | (POS.) m/z: 419.2 (M + H)⁺. ¹H NMR (400 MHz, |
| | | DMSO-d₆) δ 8.79 (s, 1H), 8.48 (d, J = 7.6 Hz, 1H), |
| | | 8.38 (dd, J = 4.8, 1.6 Hz, 1H), 7.78 (d, J = 8.8 Hz, |
| | | 2H), 7.23 (d, J = 8.6 Hz, 2H), 7.19 (dd, J = 7.6, 4.8 |
| | | Hz, 1H), 6.90 (d, J = 8.6 Hz, 2H), 6.64 (t, J = 5.9 Hz, |
| | | 1H), 5.37 (p, J = 6.9 Hz, 1H), 4.23 (d, J = 5.8 Hz, |
| | | 2H), 3.73 (s, 3H), 2.38 (s, 3H), 1.43 (d, J = 6.8 Hz, |
| | | 3H). |

### Example 16

### Synthesis of 1-(4-chlorobenzyl)-3-(4-(2-(pyridin-3-ylsulfonyl)ethyl)phenyl)urea. (Compound 487)

### Preparation of 1-(4-chlorobenzyl)-3-(4-(2-(pyridin-3-ylsulfonyl)ethyl)phenyl)urea

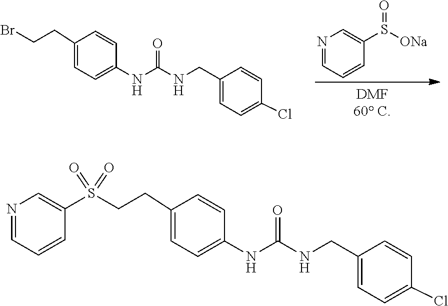

To a solution of the 1-(4-(2-bromoethyl)phenyl)-3-(4-chlorobenzyl)urea (Intermediate 41, 30 mg, 0.082 mmol, 1.0 equiv) in DMF (1 mL) was added sodium pyridine-3-sulfinate (20 mg, 0.122 mmol, 1.5 equiv) as solid. The mixture was stirred at 60° C. for 22 h. Then the reaction was directly subjected to preparative HPLC (H₂O (0.1% HCO₂H)/MeCN (0.1% HCO₂H) to yield 1-(4-chlorobenzyl)-3-(4-(2-(pyridin-3-ylsulfonyl)ethyl)phenyl)urea (8 mg, 23%) as a white solid. LCMS-ESI (POS.) m/z: 430.1 (M+H)⁺. ¹H NMR (400 MHz, DMSO-d₆) δ 9.05 (d, J=2.3 Hz, 1H), 8.90 (dd, J=4.9, 1.6 Hz, 1H), 8.52 (s, 1H), 8.30 (dt, J=8.1, 2.0 Hz, 1H), 7.68 (dd, J=8.0, 4.8 Hz, 1H), 7.38 (d, J=8.5 Hz, 2H), 7.31 (d, J=8.5 Hz, 2H), 7.26 (d, J=8.5 Hz, 2H), 7.05 (d, J=8.5 Hz, 2H), 6.61 (t, J=5.9 Hz, 1H), 4.27 (t, J=5.5 Hz, 2H), 3.77-3.68 (m, 2H), 2.89-2.79 (m, 2H).

### Biological Example 1

### NMN Fluorescence Biochemical and NAD Cellular Assay

A. Human Recombinant Enzyme Assay

Compounds described herein were assayed for their ability to stimulate the synthesis of nicotinamide mononucleotide (NMN) by the enzyme NAMPT. The human recombinant enzyme assay measures the activation of the enzyme activity by compounds using recombinant enzyme and substrates in a buffered cell-free system. The assay conditions closely mimic cellular environments. Dose responses were measured using an assay to detect the formation of nicotinamide mono-nucleotide. All experiments were performed in the 384-well format. Generally, 0.5 μL of DMSO containing varying concentrations of the test compound was mixed with 10 μL of the enzyme reagent solution. Enzyme reactions were initiated with the addition of 10 μL of a solution containing the substrates. The final assay conditions were as follows: 6 nM human NAMPT, 2.5 mM ATP, 20 μM PRPP and 150 μM nicotinamide in 50 mM HEPES, pH 7.2, 1 mM DTT, 1 mM CHAPS 50 mM NaCl, 100 mM MgCl₂. Following an incubation of 60 min at ambient temperature, 10 μL of 20% acetophenone in DMSO was added, followed by 10 μL of 2 M KOH and 40 μL of formic acid. The plates were read for fluorescence (Excitation/Emission=355 nm/460 nm) using an EnVision plate reader after 40 mins of incubation at ambient temperature. The potency measurements for compounds, are quantified and represented as AC_{1.4} (the concentration of compounds that generates 40% higher activity over basal) and EC₅₀ (concentration of the compound that gives half-maximal activation). Comparative compounds A, B, C, and D were also tested, and the data are presented in Table A. For the AC_{1.4} values, compounds designated with the letter “A” have AC_{1.4} values that are less than 0.5 μM; compounds designated with the letter “B” have AC_{1.4} values that are between 0.5 μM and 2.5 μM; and compounds designated with the letter “C” have AC_{1.4} values that are greater than 2.5 μM. For example, compounds 36 and 242 have AC_{1.4} values of 0.15 and 0.42, respectively, and are designated as “A” in Table A, and compound 167 has an AC_{1.4} value of 0.78 and is designated as “B.” As shown in Table A, comparator compound A, which has an unsubstituted phenyl ring (i.e., wherein R¹ is hydrogen) is five to ten times less potent than compounds with a halo or methoxy substituent at the R¹ position, as measured by AC_{1.4}.

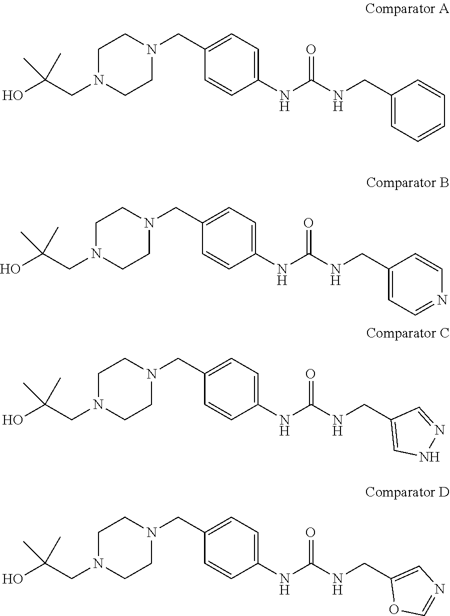 [SMILES: CC(C)(O)CN1CCN(Cc2ccc(NC(=O)NCc3ccccc3)cc2)CC1 ; CC(C)(O)CN1CCN(Cc2ccc(NC(=O)NCc3ccncc3)cc2)CC1 ; CC(C)(O)CN1CCN(Cc2ccc(NC(=O)NCc3cn[nH]c3)cc2)CC1 ; CC(C)(O)CN1CCN(Cc2ccc(NC(=O)NCc3cnco3)cc2)CC1]

TABLE A

| Compound | AC1.4 | EC50 |
|---|---|---|
| No. | Human | Human |
| Comparator A | 4.2 | 10.6 |
| Comparator B | 0.4 | 19.7 |
| Comparator C | 0.2 | 35.1 |
| Comparator D | 6.1 | 36.5 |
| 36 | 0.15 | 3.1 |
| 167 | 0.78 | 7.5 |
| 242 | 0.42 | 10.6 |
| 1 | A | |
| 2 | A | |
| 3 | A | |
| 4 | A | |
| 5 | A | |
| 6 | A | |
| 7 | A | |
| 8 | A | |
| 9 | A | |
| 10 | A | |
| 11 | A | |
| 12 | A | |
| 13 | A | |
| 14 | A | |
| 15 | A | |
| 16 | A | |
| 17 | A | |
| 18 | A | |
| 19 | A | |
| 20 | A | |
| 21 | A | |
| 22 | A | |
| 23 | A | |
| 24 | A | |
| 25 | A | |
| 26 | A | |
| 27 | A | |
| 28 | A | |
| 29 | A | |
| 30 | A | |
| 31 | A | |
| 32 | A | |
| 33 | A | |
| 34 | A | |
| 35 | A | |
| 37 | A | |
| 38 | A | |
| 39 | A | |
| 40 | A | |
| 41 | A | |
| 42 | A | |
| 43 | A | |
| 44 | A | |
| 45 | A | |
| 46 | A | |
| 47 | A | |
| 48 | A | |
| 49 | A | |
| 50 | A | |
| 51 | A | |
| 52 | A | |
| 53 | A | |
| 54 | A | |
| 55 | A | |
| 56 | A | |
| 57 | A | |
| 58 | A | |
| 59 | A | |
| 60 | A | |
| 61 | A | |
| 62 | A | |
| 63 | A | |
| 64 | A | |
| 65 | A | |
| 66 | A | |
| 67 | A | |
| 68 | A | |
| 69 | A | |
| 70 | A | |
| 71 | A | |
| 72 | A | |
| 73 | A | |
| 74 | A | |
| 75 | A | |
| 76 | A | |
| 77 | A | |
| 78 | A | |
| 79 | A | |
| 80 | A | |
| 81 | A | |
| 82 | A | |
| 83 | A | |
| 84 | A | |
| 85 | A | |
| 86 | A | |
| 87 | A | |
| 88 | A | |
| 89 | A | |
| 90 | A | |
| 91 | A | |
| 92 | A | |
| 93 | A | |
| 94 | A | |
| 95 | A | |
| 96 | A | |
| 97 | A | |
| 98 | A | |
| 99 | A | |
| 100 | A | |
| 101 | A | |
| 102 | A | |
| 103 | A | |
| 104 | A | |
| 105 | B | |
| 106 | B | |
| 107 | B | |
| 108 | B | |
| 109 | B | |
| 110 | B | |
| 111 | B | |
| 112 | B | |
| 113 | B | |
| 114 | B | |
| 115 | B | |
| 116 | B | |
| 117 | B | |
| 118 | B | |
| 119 | B | |
| 120 | B | |
| 121 | B | |
| 122 | B | |
| 123 | B | |
| 124 | B | |
| 125 | B | |
| 126 | B | |
| 127 | B | |
| 128 | B | |
| 129 | B | |
| 130 | B | |
| 131 | B | |
| 132 | B | |
| 133 | B | |
| 134 | B | |
| 135 | B | |
| 136 | B | |
| 137 | B | |
| 138 | B | |
| 139 | B | |
| 140 | B | |
| 141 | B | |
| 142 | B | |
| 143 | B | |
| 144 | B | |
| 145 | B | |
| 146 | B | |
| 147 | C | |
| 148 | C | |
| 149 | A | |
| 150 | A | |
| 151 | A | |
| 152 | A | |
| 153 | A | |
| 154 | A | |
| 155 | A | |
| 156 | A | |
| 157 | A | |
| 158 | B | |
| 159 | B | |
| 160 | B | |
| 161 | B | |
| 162 | B | |
| 163 | B | |
| 164 | B | |
| 165 | B | |
| 166 | B | |
| 168 | B | |
| 169 | B | |
| 170 | B | |
| 171 | B | |
| 172 | B | |
| 173 | B | |
| 174 | B | |
| 175 | B | |
| 176 | B | |
| 177 | B | |
| 178 | B | |
| 179 | B | |
| 180 | B | |
| 181 | B | |
| 182 | B | |
| 183 | B | |
| 184 | B | |
| 185 | B | |
| 186 | B | |
| 187 | B | |
| 188 | B | |
| 189 | B | |
| 190 | B | |
| 191 | B | |
| 192 | B | |
| 193 | B | |
| 194 | B | |
| 195 | C | |
| 196 | C | |
| 197 | C | |
| 198 | A | |
| 199 | A | |
| 200 | A | |
| 201 | A | |
| 202 | A | |
| 203 | A | |
| 204 | A | |
| 205 | A | |
| 206 | A | |
| 207 | A | |
| 208 | A | |
| 209 | A | |
| 210 | A | |
| 211 | A | |
| 212 | A | |
| 213 | A | |
| 214 | A | |
| 215 | A | |
| 216 | A | |
| 217 | A | |
| 218 | A | |
| 219 | A | |
| 220 | A | |
| 221 | A | |
| 222 | A | |
| 223 | A | |
| 224 | A | |
| 225 | A | |
| 226 | A | |
| 227 | A | |
| 228 | A | |
| 229 | A | |
| 230 | A | |
| 231 | A | |
| 232 | A | |
| 233 | A | |
| 234 | A | |
| 235 | A | |
| 236 | A | |
| 237 | A | |
| 238 | A | |
| 239 | A | |
| 240 | A | |
| 241 | A | |
| 243 | A | |
| 244 | A | |
| 245 | A | |
| 246 | A | |
| 247 | A | |
| 248 | A | |
| 249 | A | |
| 250 | A | |
| 251 | A | |
| 252 | A | |
| 253 | A | |
| 254 | A | |
| 255 | B | |
| 256 | B | |
| 257 | B | |
| 258 | B | |
| 259 | B | |
| 260 | B | |
| 261 | B | |
| 262 | B | |
| 263 | B | |
| 264 | B | |
| 265 | B | |
| 266 | B | |
| 267 | B | |
| 268 | B | |
| 269 | B | |
| 270 | B | |
| 271 | B | |
| 272 | B | |
| 273 | B | |
| 274 | B | |
| 275 | B | |
| 276 | B | |
| 277 | B | |
| 278 | B | |
| 279 | B | |
| 280 | B | |
| 281 | B | |
| 282 | B | |
| 283 | B | |
| 284 | B | |
| 285 | B | |
| 286 | B | |
| 287 | B | |
| 288 | B | |
| 289 | B | |
| 290 | B | |
| 291 | B | |
| 292 | B | |
| 293 | B | |
| 294 | B | |
| 295 | B | |
| 296 | B | |
| 297 | B | |
| 298 | B | |
| 299 | B | |
| 300 | B | |
| 301 | B | |
| 302 | B | |
| 303 | B | |
| 304 | B | |
| 305 | B | |
| 306 | B | |
| 307 | B | |
| 308 | B | |
| 309 | B | |
| 310 | B | |
| 311 | B | |
| 312 | B | |
| 313 | B | |
| 314 | B | |
| 315 | B | |
| 316 | B | |
| 317 | B | |
| 318 | B | |
| 319 | B | |
| 320 | B | |
| 321 | B | |
| 322 | B | |
| 323 | B | |
| 324 | B | |
| 325 | B | |
| 326 | B | |
| 327 | B | |
| 328 | B | |
| 329 | B | |
| 330 | B | |
| 331 | B | |
| 332 | B | |
| 333 | B | |
| 334 | B | |
| 335 | B | |
| 336 | B | |
| 337 | B | |
| 338 | B | |
| 339 | B | |
| 340 | B | |
| 341 | B | |
| 342 | B | |
| 343 | B | |
| 344 | B | |
| 345 | B | |
| 346 | B | |
| 347 | B | |
| 348 | B | |
| 349 | B | |
| 350 | C | |
| 351 | C | |
| 352 | C | |
| 353 | C | |
| 354 | C | |

B. Cellular NAD+ Modulation Assay.

The compounds described herein were also assayed for their ability to stimulate the endogenous NAMPT in a native cellular environment in the cellular NAD+ modulation assay, which measures the ability of the compound to modulate cellular NAD levels. Increased levels of NAD are expected by compounds that permeate the cells and activate the catalytic activity of the endogenous NAMPT.

Neuroblastoma SH-SY5Y cells were grown in 1:1 mixture of Eagle's Minimum Essential Medium and F12 Medium, along with 10% fetal bovine serum, in a humidified incubator with an atmosphere of 95% air and 5% C₀₂ at 37° C. The assays were initiated by plating 20 μL of SH-SY5Y cells in culture medium with 0.1% fetal bovine serum, at a density of 5000 cells per well to a 384-well Corning™ BioCoat™ Poly-D-Lysine Multiwell Plates. The plates were incubated in the 37° C. incubators for a period of 5 hours. Compounds in DMSO were added to the plates in a volume of 120 nL using the Labcyte Echo Liquid Handlers. 5 μL of a 1.5 uM Doxorubicin solution in assay medium is added to each well. The plates are then incubated for 40 hours. 30 μL of a readout-solution containing 0.2 U/mL Diaphorase enzyme, 40 uM resazurin, 10 uM FMN, 0.8 U/mL Alcohol dehydrogenase, 3% ethanol, 0.4 mg/mL bovine serum albumin, 0.2% Triton X-100 in 100 mM Tris-HCl, 30 mM EDTA, pH 8.4. The plates were read for fluorescence (Excitation/Emission=540 nm/590 nm) using an EnVision plate reader after 60 mins of incubation at ambient temperature. Table B shows the AC_{0.3}, delta recovery, and EC₅₀ data for the tested compounds Comparative compounds A, B, C, and D were also tested, and the data are presented in Table B. For the AC_{0.3} values, compounds designated with the letter “A” have AC_{0.3} values that are less than 0.5 μM; compounds designated with the letter “B” have AC_{0.3} values that are between 0.5 μM and 2.5 μM; and compounds designated with the letter “C” have AC_{0.3} values that are greater than 2.5 μM. For example, compound 36 has an AC_{0.3} value of 0.15 and is designated as “A” in Table B, and compounds 167 and 242 have AC_{0.3} values of 1.2 and 0.86, respectively, and are designated as “B.”

TABLE B

| Compound | | |
|---|---|---|
| Number | AC_{0.3} | EC₅₀ |
| Comparator A | 4.2 | 2.3 |
| Comparator B | >9.2 | >20.1 |
| Comparator C | >20.1 | >20.1 |
| Comparator D | >20.1 | >20.1 |
| 36 | 0.15 | 0.16 |
| 167 | 1.2 | 1.4 |
| 242 | 0.86 | 0.79 |
| 1 | A | |
| 2 | A | |
| 5 | A | |
| 6 | A | |
| 12 | A | |
| 13 | A | |
| 14 | A | |
| 18 | A | |
| 29 | B | |
| 33 | B | |
| 37 | A | |
| 40 | C | |
| 42 | B | |
| 50 | A | |
| 51 | C | |
| 56 | B | |
| 63 | A | |
| 68 | B | |
| 78 | A | |
| 79 | A | |
| 80 | A | |
| 86 | A | |
| 98 | A | |
| 136 | B | |
| 157 | B | |
| 159 | C | |
| 160 | A | |
| 161 | B | |
| 162 | C | |
| 165 | B | |
| 168 | C | |
| 176 | A | |
| 194 | C | |
| 199 | A | |
| 200 | A | |
| 202 | B | |
| 205 | A | |
| 206 | B | |
| 209 | B | |
| 217 | A | |
| 219 | A | |
| 232 | B | |
| 234 | B | |
| 256 | A | |
| 257 | C | |
| 264 | B | |
| 270 | B | |
| 275 | B | |
| 301 | B | |
| 302 | B | |
| 313 | B | |
| 318 | B | |

### Biological Example 2

### Bidirectional Permeability Through Caco-2 Monolayers

Caco-2 permeability was assessed for compounds described herein. As discussed previously, the Caco-2 permeability assay is commonly used to investigate human intestinal permeability and drug efflux and is an accurate predictor of in vivo absorption. Caco-2 cells (clone C2BBe1) were obtained from American Type Culture Collection (Manassas, Va.). Cell monolayers were grown to confluence on collagen-coated, microporous membranes in 12-well assay plates. Details of the plates and their certification are shown below. The permeability assay buffer was Hanks' balanced salt solution containing 10 mM HEPES and 15 mM glucose at a pH of 7.4. The buffer in the receiver chamber also contained 1% bovine serum albumin. The dosing solution concentration was 5 μM for the test article in the assay buffer. Cell monolayers were dosed on the apical side (A-to-B) or basolateral side (B-to-A) and incubated at 37° C. with 5% CO₂ in a humidified incubator. Samples were taken from the donor and receiver chambers at 120 minutes. Each determination was performed in duplicate. The flux of co-dosed lucifer yellow was also measured for each monolayer to ensure no damage was inflicted to the cell monolayers during the flux period. All samples were assayed by LC-MS/MS (Waters ACQUITY UPLC® BEH Phenyl 30×2.1 mm, 1.7 μm) using electrospray ionization, using ammonium formate as the buffer (25 mM, pH 3.5).

The apparent permeability (Pₐₚₚ) and percent recovery were calculated as follows:

Pₐₚₚ=(dCᵣ/dt)×Vᵣ/(A×C_{A}) (1)

Percent Recovery=100×((Vᵣ×Cᵣ^{final})+(V_{d}×C_{d}^{final}))/(V_{d}×C_{N}) (2)

### Wherein:

dCr/dt is the slope of the cumulative concentration in the receiver compartment versus time in μM s-1;
Vᵣ is the volume of the receiver compartment in cm³;
V_{d} is the volume of the donor compartment in cm³;
A is the area of the insert (1.13 cm² for 12-well plates);
C_{A} is the average of the nominal dosing concentration and the measured 120-minute donor concentration in M;
C_{N} is the nominal concentration of the dosing solution in M;
Cᵣ^{final} is the cumulative receiver concentration in M at the end of the incubation period;
C_{d}^{final} is the concentration of the donor in M at the end of the incubation period; and
Efflux ratio (ER) is defined as Pₐₚₚ (B-to-A)/Pₐₚₚ (A-to-B).

Data for compounds tested are presented in Table C. Comparative compounds B, C, and D were also tested. As shown in the provided data, tested compounds having a halo or methoxy substituent at the R¹ position demonstrate improved permeability compared with Comparator compounds B, C, and D.

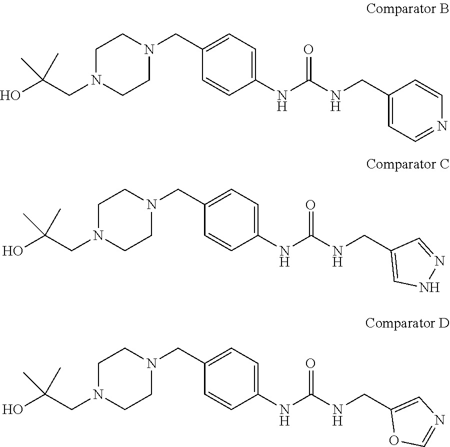 [SMILES: CC(C)(O)CN1CCN(Cc2ccc(NC(=O)NCc3ccncc3)cc2)CC1 ; CC(C)(O)CN1CCN(Cc2ccc(NC(=O)NCc3cn[nH]c3)cc2)CC1 ; CC(C)(O)CN1CCN(Cc2ccc(NC(=O)NCc3cnco3)cc2)CC1]

TABLE C.1

| Compound | | Recov- | Pₐₚₚ (10⁻⁶ cm/s) | | | Efflux |
|---|---|---|---|---|---|---|
| number | Direction | ery (%) | R1 | R2 | AVG | Ratio |
| Comparator | A-to-B | 111 | 0.148 | 0.128 | 0.138 | 16.7 |
| B | B-to-A | 108 | 2.19 | 2.42 | 2.31 | |
| Comparator | A-to-B | 110 | 0.135 | 0.132 | 0.134 | 3.98 |
| C | B-to-A | 104 | 0.531 | 0.533 | 0.532 | |
| Comparator | A-to-B | 90.6 | 0.273 | 0.287 | 0.280 | 10.8 |
| D | B-to-A | 94.4 | 2.49 | 3.57 | 3.03 | |
| 36 | A-to-B | 87.9 | 17.4 | 15.2 | 16.3 | 1.66 |
| | B-to-A | 102 | 22.6 | 31.7 | 27.2 | |
| 167 | A-to-B | 102 | 9.84 | 9.17 | 9.50 | 3.06 |
| | B-to-A | 107 | 25.5 | 32.6 | 29.0 | |
| 242 | A-to-B | 91.1 | 8.08 | 7.47 | 7.78 | 3.45 |
| | B-to-A | 103 | 24.3 | 29.3 | 26.8 | |

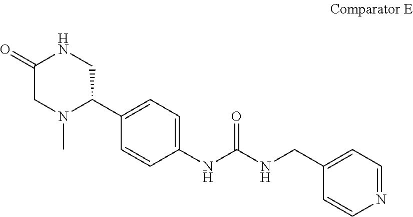 [SMILES: CN1CC(=O)NC[C@@H]1c1ccc(NC(=O)NCc2ccncc2)cc1]

TABLE C.2

| Compound | | Recovery | Pₐₚₚ (10⁻⁶ cm/s) | | | Efflux |
|---|---|---|---|---|---|---|
| number | Direction | (%) | R1 | R2 | AVG | Ratio |
| 392 | A-to-B | 93.66 | 4.34 | 4.44 | 4.39 | 5.53 |
| | B-to-A | 92.79 | 26.23 | 22.36 | 24.3 | |
| 399 | A-to-B | 102 | 2.41 | 2.42 | 2.41 | 7.86 |
| | B-to-A | 103 | 18.93 | 18.99 | 18.96 | |
| 398 | A-to-B | 99.8 | 3.43 | 3.33 | 3.37 | 6.69 |
| | B-to-A | 102 | 22.8 | 22.22 | 22.51 | |
| Comparator | A-to-B | 98.7 | 0.35 | 0.41 | 0.38 | 10.98 |
| E | B-to-A | 96.7 | 4.19 | 4.05 | 4.12 | |

### Biological Example 3

### Oral Pharmacokinetics

In vivo pharmacokinetics (PK) was assessed for compounds described herein in male C57BL/6 mice and male Sprague Dawley rats.

### A. Pharmacokinetics of Compounds in Male C57BL 6 Mice Following Intravenous and Oral Administration

Pharmacokinetics of compounds were determined in male C57BL/6 mice following a bolus IV dose at 1.0 mg/kg and a single PO dose at 1 mg/kg. Fifteen mice were used for each group in a sparse sampling design. Blood samples were taken up to 24 hr postdose. Concentrations in plasma were determined using a LC/MS/MS method.

Male C57BL/6 mice were obtained from Charles River Laboratories (Hollister, Calif.). Animals were housed in polycarbonate cages in unidirectional air flow rooms on a 12 hr light/dark cycle. Animals were acclimated a minimum of three days prior to PK studies. Food (Lab Diet 5001 rodent diet) and water were available ad libitum during the acclimation period and during the study, except during study procedures. All in vivo experiments were performed in compliance with the IACUC protocol, appropriate guidelines of the test facility, and animal welfare regulations.

A group of 15 mice received 1.0 mg/kg of compound intravenously via injection into the tail vein. The IV dose volume was 5 mL/kg. The IV dose solution was prepared in 10% DMA/20% PG/70% HPβCD solution (40% w/v aqueous HPβCD) at a concentration of 0.2 mg/mL. Another group of 15 mice received the compounds by oral gavage at 1 mg/kg. The oral dose volume was 5 mL/kg. The oral dosing suspension was prepared by suspending the compound in 0.5% HPMC/0.1% Tween 80 in water at a concentration 0.2 mg/mL. Concentrations of IV and PO doses were measured at the end of the study. Pharmacokinetic parameters were calculated using the nominal dose values if the measured values were within 20% of the nominal values.

Sparse blood samples were collected from groups of three mice via retro-orbital bleeding, placed into a K₂EDTA microtainer tube and maintained on ice until centrifugation to obtain plasma. Each designated group of mice were bled at two-time points. The time points were predose (PO only), 5 (IV only), 15, 30 min, 1, 2, 4, 6, 8 and 24 hr postdose. Blood samples were centrifuged for 5 min at 14,000 rpm (20,800 g) in a refrigerated Eppendorf Model 5804 R centrifuge and the collected plasma was transferred to an Eppendorf™ tube and stored at −80° C. until analysis.

Plasma samples were analyzed for compound concentrations using an LC/MS/MS method as described below. Briefly, a 50 μL aliquot of each plasma sample was mixed with 100 μL of acetonitrile that contained compound as the internal standard (IS). The mixture was vortexed and centrifuged. The supernatant was transferred and filtered through a membrane (Pall Corporation, AcroPrep 96-well filter plate, 0.2 μm hydrophilic polypropylene membrane). Ten μL of the resulting solution was injected onto a reverse-phase C18 column and the resultant peaks detected on a SCIEX API 4000 LC/MS/MS equipped with a turbo ionspray ionization source.

Following a bolus IV dose at 1.0 mg/kg, the mean plasma clearance (CL), volume of distribution (Vss), area under the curve (AUC) and elimination half-life (t½) was calculated or measured. Following a single oral dose at 1.0 mg/kg, the maximal plasma concentration (Cmax) and AUC∞ was measured or calculated. Oral bioavailability (% F) was calculated (% F=AUC(oral)/AUC(iv)×100).

Tables D-1 and D-2 show the PK parameters of compounds in male C57BL/6 mice following an IV dose of the compounds at 1.0 mg/kg, wherein AUCₗₐₛₜ stands for the area under the concentration-time curve from hour 0 to the last measurable concentration, AUC_{∞} stands for the area under the concentration-time curve extrapolated to infinity, CL is the apparent plasma clearance, Vₛₛ is the apparent volume of distribution at steady state, and t½ is the time to maximum observed concentration.

TABLE D-1

| | Compound | Compound | Compound | Compar- |
|---|---|---|---|---|
| PK Parameters | 242 | 167 | 36 | ator B |
| Dose (mg/kg) | 1.0 | 1.0 | 1.0 | 1.0 |
| AUCₗₐₛₜ | 82.4 | 93.5 | 86.7 | 46.7 |
| (min*umol/L) | | | | |
| Dose Normalized | 35.3 | 39.4 | 38.5 | 19.0 |
| AUC_{∞} | | | | |
| (min*kg*umol/ | | | | |
| L/umol) | | | | |
| CL (mL/min/kg) | 28.3 | 25.4 | 26.0 | 52.6 |
| Vₛₛ (L/kg) | 4.3 | 6.4 | 8.2 | 3.1 |
| t_{1/2} (min) | 223 | 275.5 | 303 | 150 |

TABLE D-2

| | Compound | Compound | Compound | Compar- |
|---|---|---|---|---|
| PK Parameters | 392 | 399 | 398 | ator E |
| Dose (mg/kg) | 1 | 1 | 1 | 1 |
| AUCₗₐₛₜ | 813 | 507 | 564.8 | 107 |
| (min*umol/L) | | | | |
| Dose Normalized | 311 | 188 | 204 | 36.5 |
| AUC_{∞} | | | | |
| (min*kg*umol/ | | | | |
| L/umol) | | | | |
| CL (mL/min/kg) | 3.21 | 5.32 | 4.9 | 27.3 |
| Vₛₛ (L/kg) | 0.83 | 0.76 | 1.07 | 0.65 |
| t_{1/2} (min) | 317 | 240 | 279 | 75 |

Tables E-1 and E-2 show the PK parameters of compounds in male C57BL/6 mice following an oral dose of the compounds at 1.0 mg/kg, wherein Cₘₐₓ, is the maximum observed concentration, tₘₐₓ is the time to maximum observed concentration, AUCₗₐₛₜ stands for the area under the concentration-time curve from hour 0 to the last measurable concentration, AUC_{∞} stands for the area under the concentration-time curve extrapolated to infinity, % F is the percentage of oral bioavailability, and t½ is the time to maximum observed concentration.

TABLE E-1

| | Compound | Compound | Compound | Compar- |
|---|---|---|---|---|
| PK Parameters | 242 | 167 | 36 | ator B |
| Dose (mg/kg) | 1.0 | 1.0 | 1.0 | 1.0 |
| Cₘₐₓ (μmol/L) | 0.17 | 0.14 | 0.10 | 0.03 |
| tₘₐₓ (min) | 60 | 60 | 240 | 120 |
| AUCₗₐₛₜ | 40.2 | 75.0 | 67.4 | 6.2 |
| (min*umol/L) | | | | |
| Dose normalized | 21.5 | 31.5 | 29.8 | 3.0 |
| AUC_{∞} | | | | |
| (min*kg*umol/ | | | | |
| L/umol) | | | | |
| % F | 49% | 80% | 78% | 16% |
| t_{1/2} (min) | 202 | 223 | 269 | 175 |

TABLE E-2

| | Compound | Compound | Compound | Compar- |
|---|---|---|---|---|
| PK Parameters | 392 | 398 | 398 | ator E |
| Dose (mg/kg) | 1 | 1 | 1 | 1 |
| Cₘₐₓ (μmol/L) | 1.73 | 3.09 | 2.32 | 0.03 |
| tₘₐₓ (min) | 60 | 30 | 60 | 30 |
| AUCₗₐₛₜ | 987 | 647 | 848 | 12.4 |
| (min*umol/L) | | | | |
| Dose normalized | 373 | 238 | 304 | 4.2 |
| AUC_{∞} | | | | |
| (min*kg*umol/ | | | | |
| L/umol) | | | | |
| % F | >100% | >100% | >100% | 12% |
| t_{1/2} (min) | 221 | 208 | 204 | 177 |

### B. Pharmacokinetics of Compounds in Male Sprague Dawley Rats Following Intravenous and Oral Administration

Pharmacokinetics of compounds was studied in male Sprague Dawley rats following IV and PO administration. Three rats were used in each dose group. Serial blood samples were taken up to 24 hours post-dose. Concentrations of compound in plasma were determined using a LC/MS/MS method. The mean calculated pharmacokinetic parameters are summarized in Tables F and G.

Male Sprague Dawley rats with surgically implanted cannula at the jugular vein were obtained from Charles River Laboratories (Hollister, Calif.). All cannulae were locked using heparin dextrose solution. Animals were housed individually in polycarbonate cages in unidirectional air flow rooms on a 12 h light/dark cycle. Animals were acclimated a minimum of three days prior to PK studies. Food (Lab Diet 5001 rodent diet) and water were available ad libitum during the acclimation period and during the study, except during study procedures. All in vivo experiments were performed in compliance with the IACUC protocol, appropriate guidelines of the test facility (Cytokinetics, Inc), and animal welfare regulations.

Three rats were dosed IV via a bolus injection via the jugular vein cannula. Three rats were dosed by oral gavage. Vehicles for dosing were: (Vehicle A for IV studies) 10% DMA: 50% PG: 40% aqueous HPβCD; (Vehicle B for PO studies) was 0.5% HPMC/0.1% Tween 80. Blood samples were collected in Microtainer™ plasma tubes (K₃EDTA) from the jugular vein cannula at predose, 5 (IV only), 15, 30 min, 1, 2, 4, 6, and 24 h post-dose. Blood volumes were replaced with an equal amount of sterile 0.9% saline. Blood samples were centrifuged for 5 min at 14,000 rpm (20,800 g) in a refrigerated Eppendorf™ Model 5804 R centrifuge and the collected plasma was transferred to an Eppendorf™ tube and stored at −80° C. for subsequent analysis.

The IV dose solution was prepared in 10% DMA/50% PEG400/40% HPβCD solution (40% w/v aqueous HPβCD) at a concentration of 1 mg/mL. The oral dose suspension was prepared by suspending compound in 0.5% HPMC/0.1% Tween 80 in water. Concentrations of IV and PO doses were measured at the end of the study. Pharmacokinetic parameters were calculated using the nominal dose values if the measured values were within 20% of the nominal values.

Plasma samples were analyzed for compound concentrations using the LC/MS/MS method described below. Briefly, a 50 μL aliquot of each plasma sample was mixed with 100 μL of acetonitrile that contained compound as the internal standard. The mixture was vortexed and centrifuged. The supernatant was transferred and filtered through a membrane (Pall Corporation, AcroPrep 96-well filter plate, 0.2 m hydrophilic polypropylene membrane). Ten L of the resulting solution was injected onto a reverse-phase C18 column and the resultant peaks detected on a SCIEX API 4000 LC/MS/MS equipped with a turbo ionspray ionization source.

Following a bolus IV dose at 1.0 mg/kg, the mean plasma clearance (CL), volume of distribution (Vss), area under the curve (AUC) and elimination half-life (t½) was calculated or measured. Following a single oral dose at 1.0 mg/kg, the maximal plasma concentration (Cmax) and AUC∞ was measured or calculated. Oral bioavailability (% F) was calculated (% F=AUC(oral)/AUC(iv)×100).

Table F shows the PK parameters of compounds in male Sprague Dawley rats following an IV dose of the compounds at 1.0 mg/kg, wherein AUCₗₐₛₜ stands for the area under the concentration-time curve from hour 0 to the last measurable concentration, AUC_{∞} stands for the area under the concentration-time curve extrapolated to infinity, CL is the apparent plasma clearance, Vₛₛ is the apparent volume of distribution at steady state, and t½ is the time to maximum observed concentration.

TABLE F

| | Compound | Comparator |
|---|---|---|
| PK Parameters | 242 | Compound B |
| Dose (mg/kg) | 1.0 | 1.0 |
| AUCₗₐₛₜ (min*umol/L) | 38.1 | 38.1 |
| Dose normalized | 17.1 | 17.1 |
| AUC_{∞} (min*kg*umol/ | | |
| L/umol) | | |
| CL (mL/min/kg) | 58.7 | 58.5 |
| Vₛₛ (L/kg) | 6.3 | 8.6 |
| t_{1/2} (min) | 94.2 | 133 |

Table G shows the PK parameters of compounds in male Sprague Dawley rats following an oral dose of the compounds at 1.0 mg/kg, wherein Cₘₐₓ is the maximum observed concentration, tₘₐₓ is the time to maximum observed concentration, AUCₗₐₛₜ stands for the area under the concentration-time curve from hour 0 to the last measurable concentration, AUC_{∞} stands for the area under the concentration-time curve extrapolated to infinity, % F is the percentage of oral bioavailability, and t½ is the time to maximum observed concentration.

TABLE G

| | Compound | Comparator |
|---|---|---|
| PK Parameters | 242 | Compound B |
| Dose (mg/kg) | 1.0 | 1.0 |
| Cₘₐₓ (μmol/L) | 0.13 | 0.01 |
| tₘₐₓ (min) | 5 | 120 |
| AUCₗₐₛₜ (min*umol/L) | 31.1 | 1.1 |
| Dose normalized | 16.3 | — |
| AUC_{∞} (min*kg*umol/ | | |
| L/umol) | | |
| % F | 95% | <5% |
| t_{1/2} (min) | 119 | — |

For both mice and rat studies, sample concentrations below the limit of quantification (BLQ) were treated as zero for pharmacokinetic calculations.

Composite pharmacokinetic parameters were estimated from a maximum of two sampling points per mouse and three mice per sampling point and the sparse data option of WinNonlin was used for noncompartmental analysis of the concentration-time data (Phoenix WinNonLin software, version 64; Pharsight, Mountain View, Calif.).

The elimination rate constant (k) was calculated as the absolute value of the slope of the linear regression of logarithm of the concentration versus time for the last three data points of the concentration-time profiles. Apparent elimination half-life (t½) values were calculated as ln(2)/k. Area under the concentration-time curve (AUC) values were estimated using linear trapezoidal method. AUCₗₐₛₜ values were calculated from the dosing time to the last measurable concentration. AUC_{∞} values were calculated as the sum of the corresponding AUCₗₐₛₜ and the ratio of the last detectable concentration divided by k. Plasma clearance (CL) was calculated from Dose/AUC_{∞}. Volume of distribution at steady state (Vₛₛ) was calculated from MRT_{∞}×CL. Maximum concentration (Cₘₐₓ) and time to reach Cₘₐₓ (tₘₐₓ) were recorded as observed. Bioavailability was calculated AUC_{∞,po}/AUC_{∞,iv}×100% where AUC was the dose normalized AUC value.

1. A compound of Formula (II) 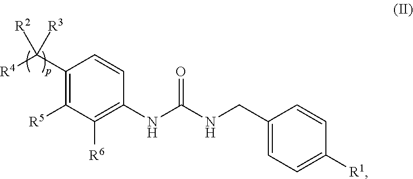
or a pharmaceutically acceptable salt thereof, wherein:
R¹ is halo or methoxy;
R⁶ is hydrogen or halo; and
p is 0 or 1, wherein
when p is 1,
R² is hydrogen or C₁-C₆ alkyl or is taken together with Z⁴ and the intervening atoms to form a 4-6 membered heterocycloalkyl or heterocycloalkenyl ring;
R³ is hydrogen or C₁-C₆ alkyl;
R⁴ is
a) Z¹NR^{a}C(O)—,
b) Z²C(O)NR^{b}—,
c) Z³(CR^{c}R^{d})ₘNR^{e}—,
d) Z⁴S(O)₂(CH₂)ₙ—,
e) Z⁵OC(O)—,
f) NR^{f}R^{g}C(O)—,
g) 5- to 10-membered heteroaryl optionally substituted with one or more independently selected C₁-C₆ alkyl or C₃-C₆ cycloalkyl substituents,
h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl optionally substituted with one or more independently selected oxo, 5- to 6-membered heteroaryl optionally substituted with one or more independently selected halo or —C₁-C₆ alkyl substituents, and C₃-C₆ cycloalkyl,
i) Z⁶S(O)₂N(R^{s})—,
j) Z⁷N(R^{t})S(O)₂—, or
k) Z⁸—O—(CH₂)_{q}—; wherein
R^{a} and R^{e} are each independently hydrogen or C₁-C₆ alkyl;
R^{b} is hydrogen or C₁-C₆ alkyl or is taken together with R⁵ and the intervening atoms to form a 5- to 6-membered heterocycloalkyl or heterocycloalkenyl ring;
R^{c} and R^{d} are each independently hydrogen or C₁-C₆ alkyl, or R^{c} and R^{d} together with the carbon to which they are attached form a C₃-C₆ cycloalkyl;
R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl;
each R^{h} is independently —C₁-C₆ alkyl, —O—C₁-C₆ alkyl, or C₆-C₁₂ aryl optionally substituted with one or more independently selected halo substituents;
each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl;
each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl;
each R^{j}, R^{k}, R^{m}, Rⁿ, R^{o}, R^{p}, R^{q}, and R^{r} is independently hydrogen or C₁-C₆ alkyl;
R^{s} is hydrogen or —C₁-C₆ alkyl;
R^{t} is hydrogen or —C₁-C₆ alkyl;
m is 0 or 1;
n is 0, 1, or 2; and
q is 0 or 1;
Z¹ and Z⁵ are each independently R^{z};
Z² and Z³ are each independently hydrogen or R^{z};
Z⁴ is hydrogen or R^{z} or is taken together with R² and the intervening atoms to form a 4-6 membered heterocycloalkyl or heterocycloalkenyl ring;
Z⁶ is selected from the group consisting of 5- to 6-membered heterocycloalkyl or heterocycloalkenyl, 5- to 6-membered heteroaryl, and C₁-C₆ alkyl;
Z⁷ is C₆-C₁₂ aryl;
Z⁸ is selected from the group consisting of 5- to 6-membered heteroaryl and C₃-C₆ cycloalkyl, and
R^{z} is selected from the group consisting of:
a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy;
b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
c) C₁-C₆ alkoxy;
d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R^{w} is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
e) C₆-C₁₂ aryl; and
f) 5- to 10-membered heteroaryl optionally substituted with one or more independently selected C₁-C₆ alkyl substituents; and
R⁵ is hydrogen, halo, or is taken together with R^{b} and the intervening atoms form a 5- to 6-membered heterocycloalkyl or heterocycloalkenyl ring, provided that
(1) when R⁴ is Z¹NR^{a}C(O)—, Z¹ is other than methyl, unsubstituted cyclopropyl, —C(CH₃)₂CH₂OH, and —CH₂-thiofuran;
(2) R⁴ is other than 4-methylpiperazinyl, 4-phenylpiperazinyl, 4-pyridylpiperazinyl, 4-(furanylmethyl)piperazinyl, 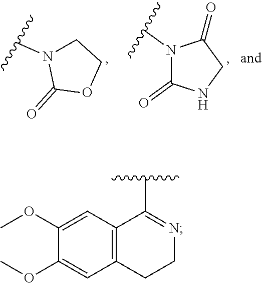 [SMILES: *N1CCOC1=O ; *N1C(=O)CNC1=O ; *C1=NCCc2cc(OC)c(OC)cc21]
and
(3) the compound of Formula (II) is not a compound of Table 1X; and
when p is 0, R⁴ is
l) 3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents,
m) 3- to 6-membered heterocycloalkyl or heterocycloalkenyl comprising exactly one annular heteroatom, which is an oxygen atom, wherein the 3- to 6-membered heterocycloalkyl or heterocycloalkenyl is optionally substituted with one or more independently selected oxo or —C₁-C₆ alkyl substituents,
n) 3- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents,
o) 5-membered heterocycloalkyl or heterocycloalkenyl comprising exactly two annular heteroatoms, one of which is a nitrogen atom and the other of which is an oxygen atom, wherein the 5-membered heterocycloalkyl or heterocycloalkenyl is optionally substituted with one or more independently selected oxo, C₁-C₆ alkyl, or —S(O)₂—(C₁-C₆ alkyl) substituents,
p) 6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, C₁-C₆ alkyl, or —S(O)₂—(C₁-C₆ alkyl) substituents,
q) 5-membered heteroaryl comprising exactly two annular heteroatoms, one of which is a nitrogen atom and the other of which is an oxygen atom, wherein the 5-membered heteroaryl is substituted with exactly one methyl substituent,
r) 5-membered heteroaryl comprising exactly two annular heteroatoms, both of which are nitrogen atoms, wherein the 5-membered heteroaryl is substituted with one or more methyl substituents,
s) 6-membered heteroaryl comprising one or two annular heteroatoms and optionally substituted with one or more methyl substituents, wherein the 6-membered heteroaryl is other than 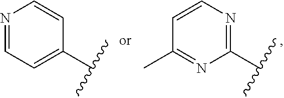
t) Z⁹—S(O)₂—,
u) Z¹⁰—S(O)₂—NH—,
v) Z¹¹—C(O)—NH—,
w) Z¹²—CH₂—O—,
x) Z¹³—O—,
y) Z¹⁴—C(H)(C₁-C₆ alkyl)-NH—C(O)—,
z) 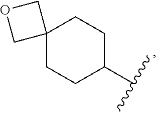 [SMILES: *C1CCC2(CC1)COC2]
or
aa) 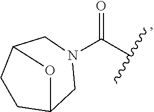
wherein
Z⁹ is selected from the group consisting of cyclopropyl, C₆-C₁₂ aryl, 3- to 10-membered heterocycloalkyl or hetercycloalkenyl optionally substituted with one or more independently selected R^{A} substituents, —NH(C₁-C₆ alkyl), —NH₂ substituted with one or more independently selected R^{B} substituents, and C₁-C₆ alkyl optionally substituted with one or more independently selected R^{C} substituents, provided that Z⁹ is other than 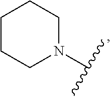 [SMILES: *N1CCCCC1]
unsubstituted methyl, or unsubstituted ethyl, wherein:
R^{A} is —C₁-C₆ alkyl or —CN; and
R^{B} is (i) —C₁-C₆ alkyl-(5- to 10-membered heteroaryl), or (ii) 5- to 10-membered heteroaryl optionally substituted with one or more independently selected C₆-C₁₂ aryl; and
R^{C} is 3- to 8-membered heterocycloalkyl or heterocycloalkenyl;
Z¹⁰ is C₁-C₆ alkyl substituted with one or more independently selected C₆-C₁₂ aryl substituents;
Z¹¹ is selected from the group consisting of C₃-C₁₀ cycloalkyl and C₁-C₆ alkyl substituted with one or more independently selected 3- to 10-membered heterocycloalkyl or hetercycloalkenyl substituents, provided that, when Z¹¹ is cyclopropyl, then R¹ is other than methoxy;
Z¹² is selected from the group consisting of C₆-C₁₂ aryl, 5- to 10-membered heteroaryl, 3- to 10-membered heterocycloalkyl or heterocycloalkenyl, C₁-C₆ alkyl substituted with one or more independently selected 3- to 10-membered heterocycloalkyl or hetercycloalkenyl substituents or 5- to 10-membered heteroaryl substituents, and —C(O)-(3- to 10-membered heterocycloalkyl or heterocycloalkenyl);
Z¹³ is 5- to 10-membered heteroaryl substituted with one or more independently selected —C(O)—NH(C₁-C₆ alkyl) substituents; and
Z¹⁴ is 5- to 10-membered heteroaryl optionally substituted with one or more independently selected C₁-C₆ alkyl substituents; and
R⁵ is hydrogen.

2. The compound of claim 1, or a pharmaceutically acceptable salt thereof, wherein R¹ is halo.

3. The compound of claim 1 or claim 2, or a pharmaceutically acceptable salt thereof, wherein R¹ is Cl or F.

4. The compound of claim 1, or a pharmaceutically acceptable salt thereof, wherein R¹ is methoxy.

5. The compound of any one of claims 1-4, or a pharmaceutically acceptable salt thereof, wherein p is 1.

6. The compound of any one of claims 1-5, or a pharmaceutically acceptable salt thereof, wherein the compound of Formula (II) is a compound of Formula (I-G) 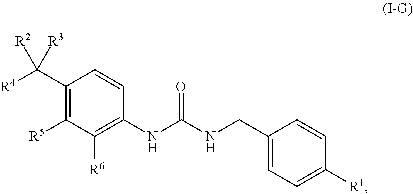
or a pharmaceutically acceptable salt thereof, wherein:
R¹ is halo or methoxy;
R² is hydrogen or C₁-C₆ alkyl or is taken together with Z⁴ and the intervening atoms to form a 4-6 membered heterocycloalkyl or heterocycloalkenyl ring;
R³ is hydrogen or C₁-C₆ alkyl;
R⁴ is
a) Z¹NR^{a}C(O)—,
b) Z²C(O)NR^{b}—,
c) Z³(CR^{c}R^{d})ₘNR^{e}—,
d) Z⁴S(O)₂(CH₂)ₙ—,
e) Z⁵OC(O)—,
f) NR^{f}R^{g}C(O)—,
g) 5- to 10-membered heteroaryl optionally substituted with one or more independently selected C₁-C₆ alkyl or C₃-C₆ cycloalkyl substituents,
h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl optionally substituted with one or more independently selected oxo, 5- to 6-membered heteroaryl optionally substituted with one or more independently selected halo or —C₁-C₆ alkyl substituents, and C₃-C₆ cycloalkyl,
i) Z⁶S(O)₂N(R^{s})—,
j) Z⁷N(R^{t})S(O)₂—, or
k) Z⁸—O—(CH₂)_{q}—; wherein
R^{a} and R^{e} are each independently hydrogen or C₁-C₆ alkyl;
R^{b} is hydrogen or C₁-C₆ alkyl or is taken together with R⁵ and the intervening atoms to form a 5- to 6-membered heterocycloalkyl or heterocycloalkenyl ring;
R^{c} and R^{d} are each independently hydrogen or C₁-C₆ alkyl, or R^{c} and R^{d} together with the carbon to which they are attached form a C₃-C₆ cycloalkyl;
R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl;
each R^{h} is independently —C₁-C₆ alkyl, —O—C₁-C₆ alkyl, or C₆-C₁₂ aryl optionally substituted with one or more independently selected halo substituents;
each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl;
each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl;
each R^{j}, R^{k}, R^{m}, Rⁿ, R^{o}, R^{p}, R^{g}, and R^{r} is independently hydrogen or C₁-C₆ alkyl;
R^{s} is hydrogen or —C₁-C₆ alkyl;
R^{t} is hydrogen or —C₁-C₆ alkyl;
m is 0 or 1;
n is 0, 1, or 2;
q is 0 or 1;
Z² and Z³ are each independently hydrogen or R^{z};
Z⁴ is hydrogen or R^{z} or is taken together with R² and the intervening atoms to form a 4-6 membered heterocycloalkyl or heterocycloalkenyl ring;
Z⁶ is selected from the group consisting of 5- to 6-membered heterocycloalkyl or heterocycloalkenyl, 5- to 6-membered heteroaryl, and C₁-C₆ alkyl;
Z⁷ is C₆-C₁₂ aryl;
Z⁸ is selected from the group consisting of 5- to 6-membered heteroaryl and C₃-C₆ cycloalkyl, and
R^{z} is selected from the group consisting of:
a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy;
b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
c) C₁-C₆ alkoxy;
d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R¹¹ is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
e) C₆-C₁₂ aryl; and
f) 5- to 10-membered heteroaryl optionally substituted with one or more independently selected C₁-C₆ alkyl substituents;
R⁵ is hydrogen, halo, or is taken together with R^{b} and the intervening atoms form a 5- to 6-membered heterocycloalkyl or heterocycloalkenyl ring; and
R⁶ is hydrogen or halo, Z¹ and Z⁵ are each independently R^{z}, provided that
(1) when R⁴ is Z¹NR^{a}C(O)—, Z¹ is other than methyl, unsubstituted cyclopropyl, —C(CH₃)₂CH₂OH, and —CH₂-thiofuran;
(2) R⁴ is other than 4-methylpiperazinyl, 4-phenylpiperazinyl, 4-pyridylpiperazinyl, 4-(furanylmethyl)piperazinyl, 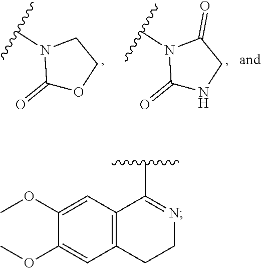 [SMILES: *N1CCOC1=O ; *N1C(=O)CNC1=O ; *C1=NCCc2cc(OC)c(OC)cc21]
and
(3) the compound of Formula (I-G) is not a compound of Table 1X.

7. The compound of any one of claims 1-5, or a pharmaceutically acceptable salt thereof, wherein the compound of Formula (II) is a compound of Formula (I) 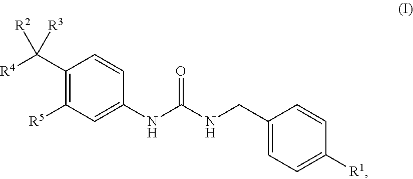
or a pharmaceutically acceptable salt thereof, wherein:
R¹ is halo or methoxy; R² is hydrogen or C₁-C₆ alkyl or is taken together with Z⁴ and the intervening atoms to form a 4-6 membered heterocycloalkyl or heterocycloalkenyl ring;
R³ is hydrogen or C₁-C₆ alkyl;
R⁴ is
a) Z¹NR^{a}C(O)—,
b) Z²C(O)NR^{b}—,
c) Z³(CR^{c}R^{d})ₘNR^{e}—,
d) Z⁴S(O)₂(CH₂)ₙ—,
e) Z⁵OC(O)—,
f) NR^{f}R^{g}C(O)—,
g) 5- to 10-membered heteroaryl optionally substituted with one or more independently selected C₁-C₆ alkyl substituents, or
h) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein
R^{a} and R^{e} are each independently hydrogen or C₁-C₆ alkyl; R^{b} is hydrogen or C₁-C₆ alkyl or is taken together with R⁵ and the intervening atoms to form a 5- to 6-membered heterocycloalkyl or heterocycloalkenyl ring; R^{c} and R^{d} are each independently hydrogen or C₁-C₆ alkyl, or R^{c} and R^{d} together with the carbon to which they are attached form a C₃-C₆ cycloalkyl; R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl; each R^{h} is independently —C₁-C₆ alkyl, —O—C₁-C₆ alkyl, or C₆-C₁₂ aryl optionally substituted with one or more independently selected halo substituents;
each R^{x} is independently selected from the group consisting of halo, —OH, —C₃-C₆ cycloalkyl, —C₁-C₆ alkoxy, —NR^{o}R^{p}, 3- to 6-membered heterocycloalkyl or heterocycloalkenyl, and 5- to 6-membered heteroaryl; each R^{y} is independently selected from the group consisting of halo, —OH, —CN, —C₁-C₆ alkoxy, —C(O)NR^{g}R^{r}, C₆-C₁₂ aryl, and 5- to 6-membered heteroaryl;
each R^{j}, R^{k}, R^{m}, Rⁿ, R^{o}, R^{p}, R^{g}, and R^{r} is independently hydrogen or C₁-C₆ alkyl;
m is 0 or 1;
n is 0, 1, or 2;
R⁵ is hydrogen or is taken together with R^{b} and the intervening atoms form a 5- to 6-membered heterocycloalkyl or heterocycloalkenyl ring;
Z¹ and Z⁵ are each independently R^{z};
Z² and Z³ are each independently hydrogen or R^{z}; Z⁴ is hydrogen or R^{z} or is taken together with R² and the intervening atoms to form a 4-6 membered heterocycloalkyl or heterocycloalkenyl ring; and
R^{z} is selected from the group consisting of: a) C₁-C₆ alkyl optionally substituted with one or more substituents independently selected from the group consisting of —OH, —CN, C₃-C₆ cycloalkyl, —NHC₁-C₆ alkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of halo, C₁-C₆ alkyl, and C₁-C₆ alkoxy; b) C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with one or more independently selected C₁-C₆ alkyl;
c) C₁-C₆ alkoxy; d) 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{w} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents; wherein each R^{w} is independently selected from the group consisting of halo, —OH, —CN, —C₁-C₆ alkoxy, —C(O)NR^{u}R^{v}, C₆-C₁₂ aryl, and 5- to 6-membered heteroaryl; and wherein R^{u} and R^{v} are each independently hydrogen or C₁-C₆ alkyl;
e) C₆-C₁₂ aryl; and
f) 5- to 10-membered heteroaryl optionally substituted with one or more independently selected C₁-C₆ alkyl substituents,
wherein
(1) when R⁴ is Z¹NR^{a}C(O)—, Z¹ is other than methyl, unsubstituted cyclopropyl, —C(CH₃)₂CH₂OH, and —CH₂-thiofuran; (2) R⁴ is other than 4-methylpiperazinyl, 4-phenylpiperazinyl, 4-pyridylpiperazinyl, 4-(furanylmethyl)piperazinyl, 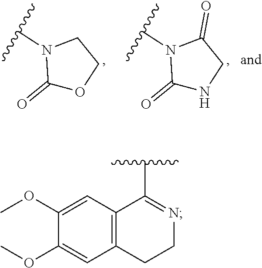 [SMILES: *N1CCOC1=O ; *N1C(=O)CNC1=O ; *C1=NCCc2cc(OC)c(OC)cc21]
and
(3) the compound of Formula (I) is not a compound of Table 1X, or a pharmaceutically acceptable salt thereof.

8. The compound of any one of claims 1-7, or a pharmaceutically acceptable salt thereof, wherein R² is hydrogen.

9. The compound of any one of claims 1-7, or a pharmaceutically acceptable salt thereof, wherein R² is C₁-C₆ alkyl.

10. The compound of any one of claims 1-9, or a pharmaceutically acceptable salt thereof, wherein R³ is hydrogen.

11. The compound of any one of claims 1-9, or a pharmaceutically acceptable salt thereof, wherein R³ is C₁-C₆ alkyl.

12. The compound of any one of claims 1-11, or a pharmaceutically acceptable salt thereof, wherein R⁴ is a 3- to 10-membered heterocycloalkyl or heterocycloalkenyl optionally substituted with one or more substituents independently selected from the group consisting of halo, oxo, —OH, —CN, —C₁-C₆ alkyl optionally substituted with one or more independently selected R^{y} substituents, —C₁-C₆ alkoxy optionally substituted with one or more independently selected halo substituents, —C(O)OC₁-C₆ alkyl, —C(O)C₁-C₆ alkyl, —S(O)₂—C₁-C₆ alkyl, C₆-C₁₂ aryl optionally substituted with one or more independently selected halo substituents, 3- to 6-membered heterocycloalkyl or heterocycloalkenyl, and 5- to 6-membered heteroaryl optionally substituted with one or more independently selected C₁-C₆ alkyl substituents.

13. The compound of any one of claims 1-12, or a pharmaceutically acceptable salt thereof, wherein R⁴ is a 4- to 6-membered heterocycloalkyl or heterocycloalkenyl optionally substituted with one or more independently selected oxo, —S(O)₂—C₁-C₆ alkyl, or —C₁-C₆ alkyl optionally substituted with —OH.

14. The compound of any one of claims 1-12, or a pharmaceutically acceptable salt thereof, wherein R⁴ is selected from the group consisting of

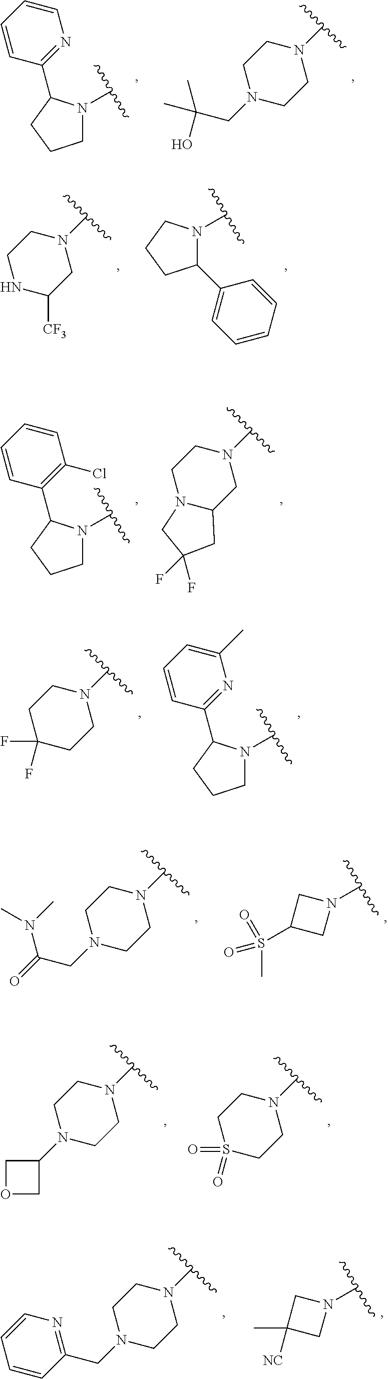

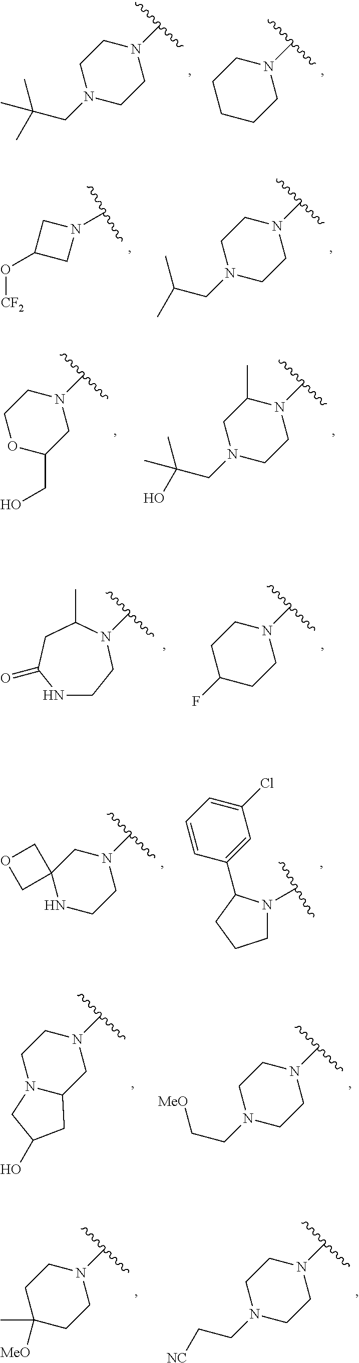

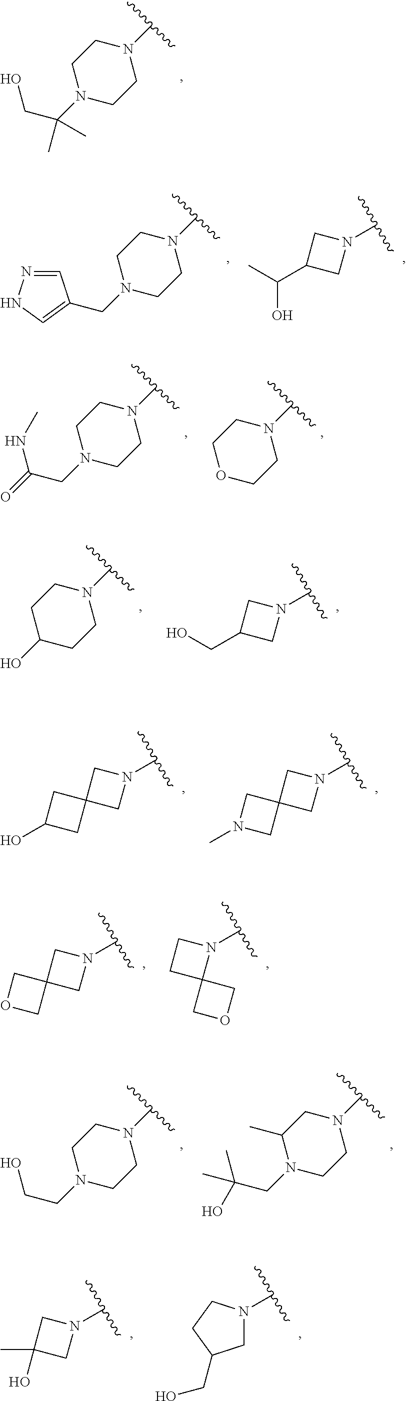

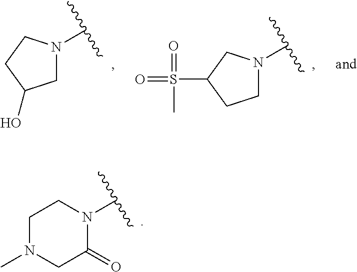

15. The compound of claim 14, or a pharmaceutically acceptable salt thereof, wherein R⁴ is

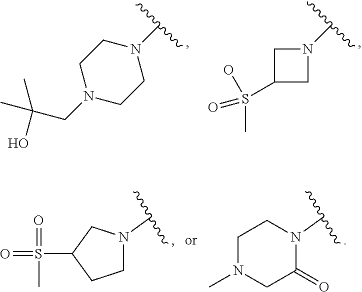

16. The compound of any one of claims 1-11, or a pharmaceutically acceptable salt thereof, wherein the compound is of Formula (I-A) 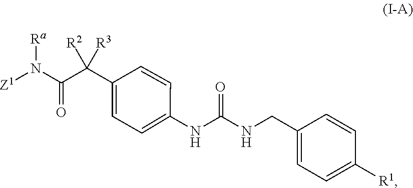
or a pharmaceutically acceptable salt thereof.

17. The compound of any one of claims 1-11 and 16, or a pharmaceutically acceptable salt thereof, wherein R^{a} is hydrogen.

18. The compound of any one of claims 1-11 and 16, or a pharmaceutically acceptable salt thereof, wherein R^{a} is C₁-C₆ alkyl.

19. The compound of any one of claims 1-11 and 16-18, or a pharmaceutically acceptable salt thereof, wherein Z¹ is selected from the group consisting of:
C₁-C₆ alkyl optionally substituted with one or more substituents independently selected from the group consisting of —OH, C₃-C₆ cycloalkyl, C₆-C₁₂ aryl, 3- to 10-membered heterocycloalkyl or heterocycloalkenyl, and 5- to 10-membered heteroaryl, wherein the C₆-C₁₂ aryl, 3- to 10-membered heterocycloalkyl or heterocycloalkenyl, and 5- to 10-membered heteroaryl are each independently optionally substituted with one or more substituents independently selected from the group consisting of C₁-C₆ alkyl and C₁-C₆ alkoxy;
C₃-C₆ cycloalkyl optionally substituted with one or more substituents independently selected from the group consisting of C₆-C₁₂ aryl, C₁-C₆ alkyl, and C₁-C₆ alkoxy optionally substituted with 5- or 10-membered heteroaryl, wherein the 5- or 10-membered heteroaryl is optionally further substituted with C₁-C₆ alkyl; and
3- to 10-membered heterocycloalkyl or heterocycloalkenyl optionally substituted with one or more substituents independently selected from the group consisting of —C₁-C₆ alkyl and —C(O)OC₁-C₆ alkyl, wherein the —C₁-C₆ alkyl is optionally substituted with C₆-C₁₂ aryl.

20. The compound of any one of claims 1-11 and 16-19, or a pharmaceutically acceptable salt thereof, wherein Z¹ is selected from the group consisting of ethyl,

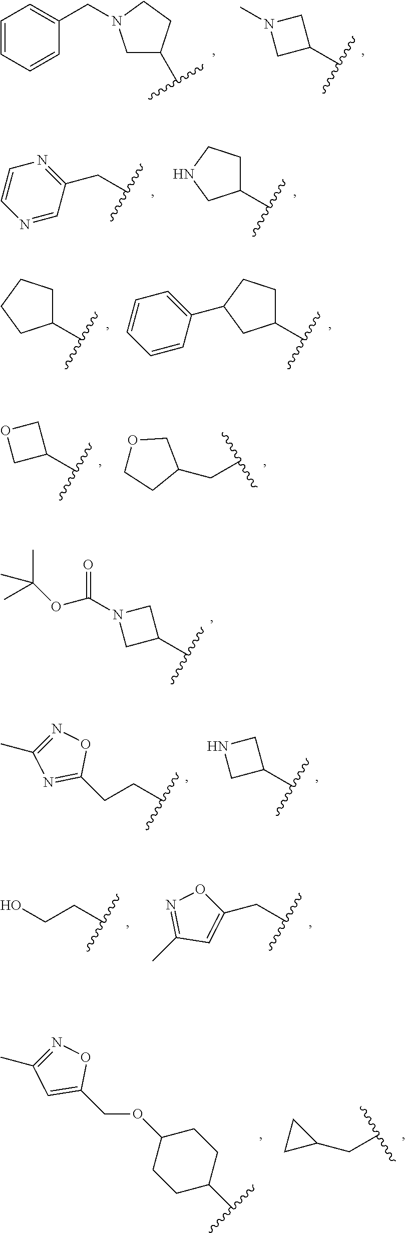

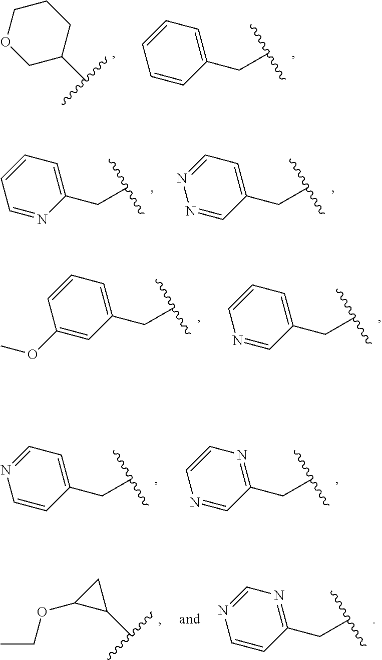

21. The compound of any one of claims 1-11, or a pharmaceutically acceptable salt thereof, wherein the compound is of Formula (I-B) 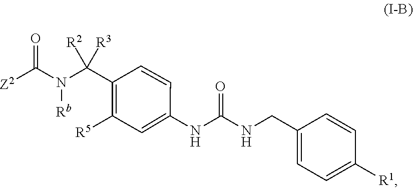
or a pharmaceutically acceptable salt thereof.

22. The compound of any one of claims 1-11 and 21, or a pharmaceutically acceptable salt thereof, wherein R^{h} is hydrogen.

23. The compound of any one of claims 1-11 and 21, or a pharmaceutically acceptable salt thereof, wherein R^{b} is C₁-C₆ alkyl.

24. The compound of any one of claims 1-11 and 21, or a pharmaceutically acceptable salt thereof, wherein R^{b} is taken together with R⁵ and the intervening atoms to form a 5- to 6-membered heterocycloalkyl or heterocycloalkenyl ring.

25. The compound of any one of claims 1-11 and 21-24, or a pharmaceutically acceptable salt thereof, wherein Z² is hydrogen.

26. The compound of any one of claims 1-11 and 21-24, or a pharmaceutically acceptable salt thereof, wherein Z² is selected from the group consisting of
C₁-C₆ alkyl optionally substituted with one or more substituents independently selected from the group consisting of C₃-C₆ cycloalkyl and 5- to 10-membered heteroaryl;
C₃-C₆ cycloalkyl optionally substituted with one or more substituents independently selected from the group consisting of C₁-C₆ alkyl and C₁-C₆ alkoxy;
C₁-C₆ alkoxy;
3- to 10-membered heterocycloalkyl or heterocycloalkenyl optionally substituted with one or more independently selected —C₁-C₆ alkyl substituents;
C₆-C₁₂ aryl; and
5- to 10-membered heteroaryl optionally substituted with one or more independently selected C₁-C₆ alkyl substituents.

27. The compound of any one of claims 1-11, 21-24, and 26, or a pharmaceutically acceptable salt thereof, wherein Z² is a 5- to 6-membered heteroaryl optionally substituted with one or more independently selected —C₁-C₆ alkyl substituents.

28. The compound of any one of claims 1-11, 21-24, 26, and 27, or a pharmaceutically acceptable salt thereof, wherein Z² is a pyridyl group optionally substituted with one or more independently selected —C₁-C₆ alkyl substituents.

29. The compound of any one of claims 1-11, 21-24, and 26, or a pharmaceutically acceptable salt thereof, wherein Z² is a 3- to 10-membered heterocycloalkyl or heterocycloalkenyl optionally substituted with one or more independently selected —C₁-C₆ alkyl substituents.

30. The compound of any one of claims 1-11, 21-24, 26, and 29, or a pharmaceutically acceptable salt thereof, wherein Z² is an azetidinyl group optionally substituted with one or more independently selected —C₁-C₆ alkyl substituents or a tetrahydrofuranyl group optionally substituted with one or more independently selected —C₁-C₆ alkyl substituents.

31. The compound of any one of claims 1-11, 21-24, and 26, or a pharmaceutically acceptable salt thereof, wherein Z² is selected from the group consisting of ethyl,

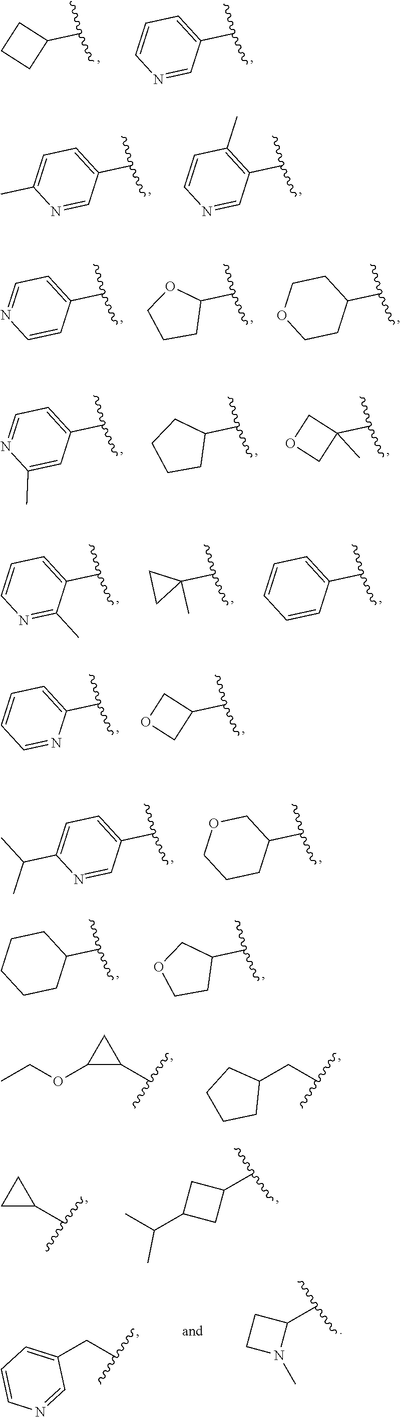

32. The compound of any one of claims 1-11, 21-24, 26, and 31, or a pharmaceutically acceptable salt thereof, wherein Z² is

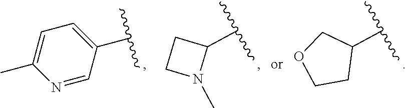

33. The compound of any one of claims 1-11, or a pharmaceutically acceptable salt thereof, wherein the compound is of Formula (I-C) 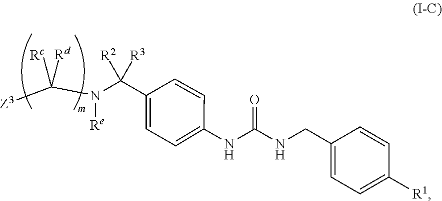
or a pharmaceutically acceptable salt thereof.

34. The compound of any one of claims 1-11 and 33, or a pharmaceutically acceptable salt thereof, wherein m is 1.

35. The compound of any one of claims 1-11 and 33, or a pharmaceutically acceptable salt thereof, wherein m is 0.

36. The compound of any one of claims 1-11 and 33-35, or a pharmaceutically acceptable salt thereof, wherein R^{c} is hydrogen.

37. The compound of any one of claims 1-11 and 33-35, or a pharmaceutically acceptable salt thereof, wherein R^{c} is C₁-C₆ alkyl.

38. The compound of any one of claims 1-11 and 33-36, or a pharmaceutically acceptable salt thereof, wherein R^{d} is hydrogen.

39. The compound of any one of claims 1-11 and 33-36, or a pharmaceutically acceptable salt thereof, wherein R^{d} is C₁-C₆ alkyl.

40. The compound of any one of claims 1-11 and 33, or a pharmaceutically acceptable salt thereof, wherein R^{c} and R^{d} together with the carbon to which they are attached form a C₃-C₆ cycloalkyl.

41. The compound of any one of claims 1-11 and 33-40, or a pharmaceutically acceptable salt thereof, wherein Reis hydrogen.

42. The compound of any one of claims 1-11 and 33-40, or a pharmaceutically acceptable salt thereof, wherein R^{e} is C₁-C₆ alkyl.

43. The compound of any one of claims 1-11 and 33-42, or a pharmaceutically acceptable salt thereof, wherein Z³ is hydrogen.

44. The compound of any one of claims 1-11 and 33-42, or a pharmaceutically acceptable salt thereof, wherein Z³ is selected from the group consisting of
C₃-C₆ cycloalkyl;
3- to 10-membered heterocycloalkyl or heterocycloalkenyl optionally substituted with —C₁-C₆ alkyl or oxo;
C₆-C₁₂ aryl; and
5- to 10-membered heteroaryl optionally substituted with one or more independently selected C₁-C₆ alkyl substituents.

45. The compound of any one of claims 1-11, 33-42, and 44, or a pharmaceutically acceptable salt thereof, wherein Z³ is 3- to 10-membered heterocycloalkyl or heterocycloalkenyl optionally substituted with —C₁-C₆ alkyl or oxo.

46. The compound of any one of claims 1-11, 33-42, 44, and 45, or a pharmaceutically acceptable salt thereof, wherein Z³ is selected from the group consisting of

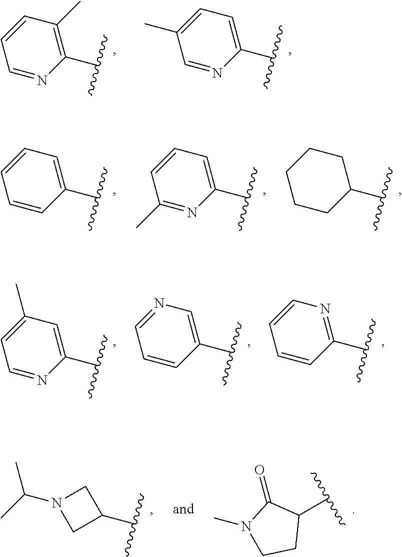

47. The compound of any one of claims 1-11, or a pharmaceutically acceptable salt thereof, wherein the compound is of Formula (I-D) 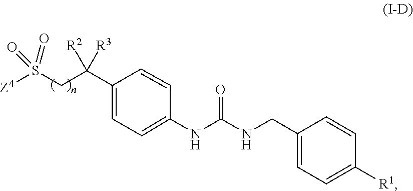
or a pharmaceutically acceptable salt thereof.

48. The compound of any one of claims 1-11 and 47, or a pharmaceutically acceptable salt thereof, wherein n is 0.

49. The compound of any one of claims 1-11 and 47, or a pharmaceutically acceptable salt thereof, wherein n is 1.

50. The compound of any one of claims 1-11 and 47, or a pharmaceutically acceptable salt thereof, wherein n is 2.

51. The compound of any one of claims 1-11 and 47-50, or a pharmaceutically acceptable salt thereof, wherein Z⁴ is hydrogen or R^{z}.

52. The compound of any one of claims 1-11 and 47-50, or a pharmaceutically acceptable salt thereof, wherein Z⁴ is C₁-C₆ alkyl.

53. The compound of any one of claims 1-11 and 47-50, or a pharmaceutically acceptable salt thereof, wherein Z⁴ is taken together with R² and the intervening atoms to form a 4-6 membered heterocycloalkyl or heterocycloalkenyl ring.

54. The compound of any one of claims 1-11, 47-50, and 53, or a pharmaceutically acceptable salt thereof, wherein 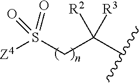
is selected from the group consisting of

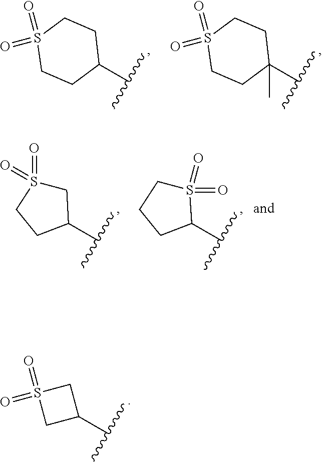

55. The compound of any one of claims 1-11, or a pharmaceutically acceptable salt thereof, wherein the compound is of Formula (I-E) 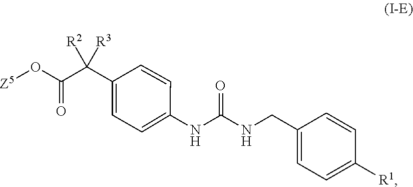
or a pharmaceutically acceptable salt thereof.

56. The compound of any one of claims 1-11 and 55, or a pharmaceutically acceptable salt thereof, wherein Z⁵ is C₁-C₆ alkyl.

57. The compound of any one of claims 1-11, 55, and 56, or a pharmaceutically acceptable salt thereof, wherein Z⁵ is ethyl.

58. The compound of any one of claims 1-11, or a pharmaceutically acceptable salt thereof, wherein the compound is of Formula (I-F) 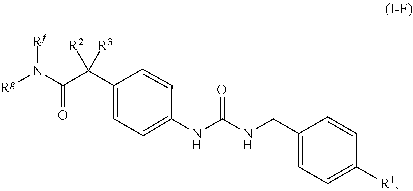
or a pharmaceutically acceptable salt thereof.

59. The compound of any one of claims 1-11 and 58, or a pharmaceutically acceptable salt thereof, wherein R^{f} and R^{g} together with the nitrogen to which they are attached form a 3- to 10-membered heterocycloalkyl or heterocycloalkenyl optionally substituted with one or more substituents independently selected from the group consisting of halo, —OH, —CN, oxo, —C₁-C₆ alkyl optionally substituted with one or more independently selected R^{x} substituents, —C₃-C₆ cycloalkyl, —C₁-C₆ alkoxy, —C(O)R^{h}, —NHC(O)OC₁-C₆ alkyl, —NR^{j}R^{k}, —C(O)NR^{m}Rⁿ, 3- to 6-membered heterocycloalkyl or heterocycloalkenyl, and 5- to 6-membered heteroaryl.

60. The compound of any one of claims 1-11, 58, and 59, or a pharmaceutically acceptable salt thereof, wherein R^{f} and R^{g} together with the nitrogen to which they are attached form a 5- to 6-membered heterocycloalkyl or heterocycloalkenyl optionally substituted with —C₁-C₆ alkyl, wherein the —C₁-C₆ alkyl is optionally substituted with —OH.

61. The compound of any one of claims 1-11 and 58-60, or a pharmaceutically acceptable salt thereof, wherein 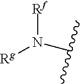
is selected from the group consisting of

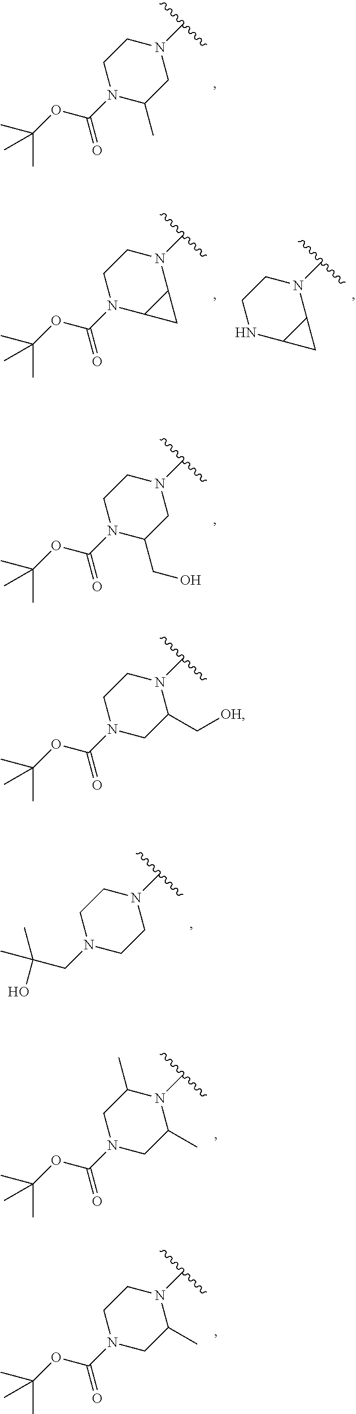

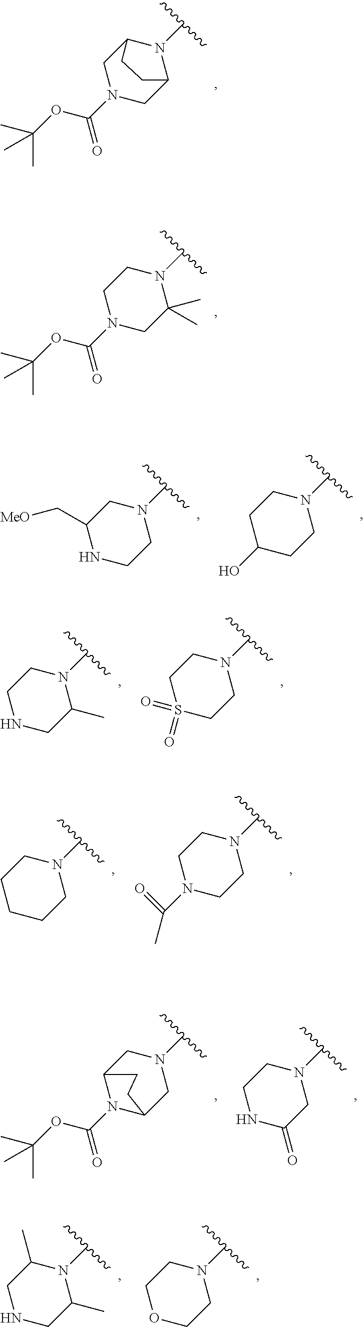

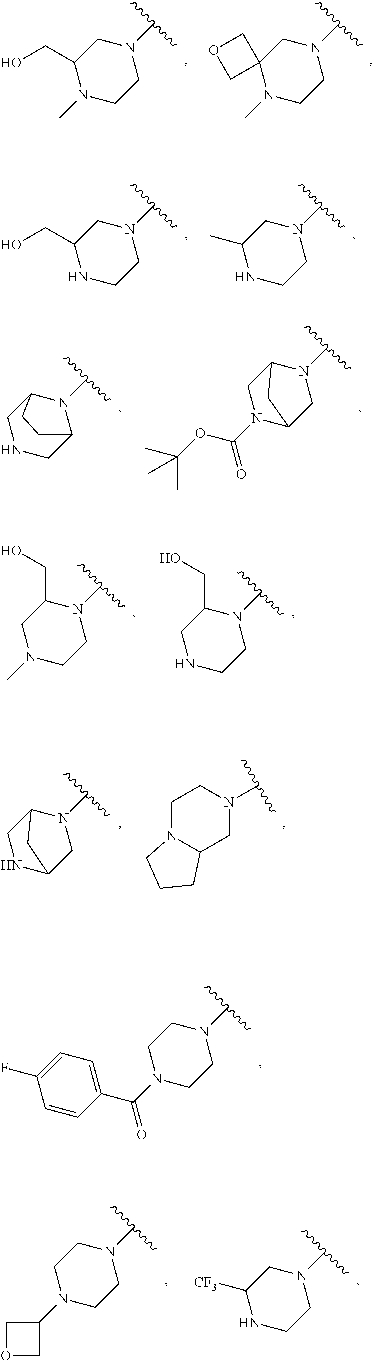

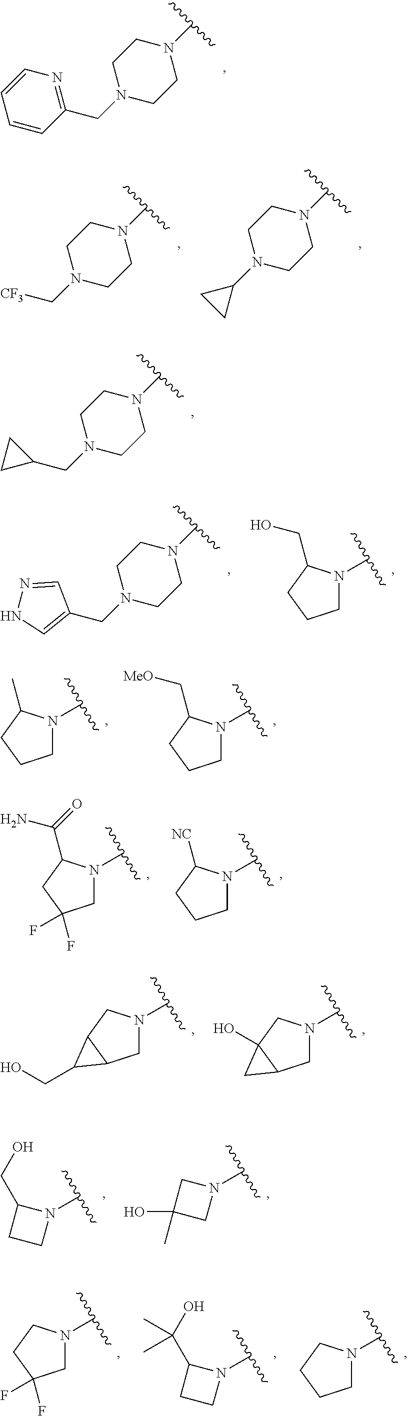

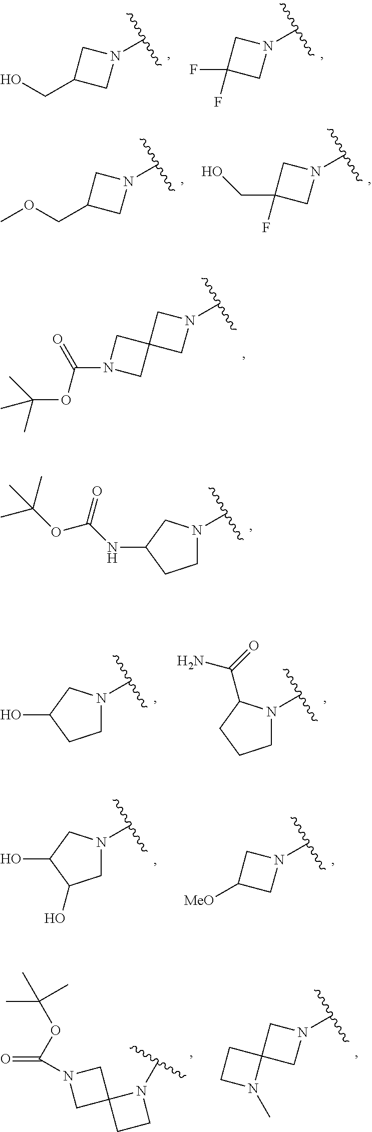

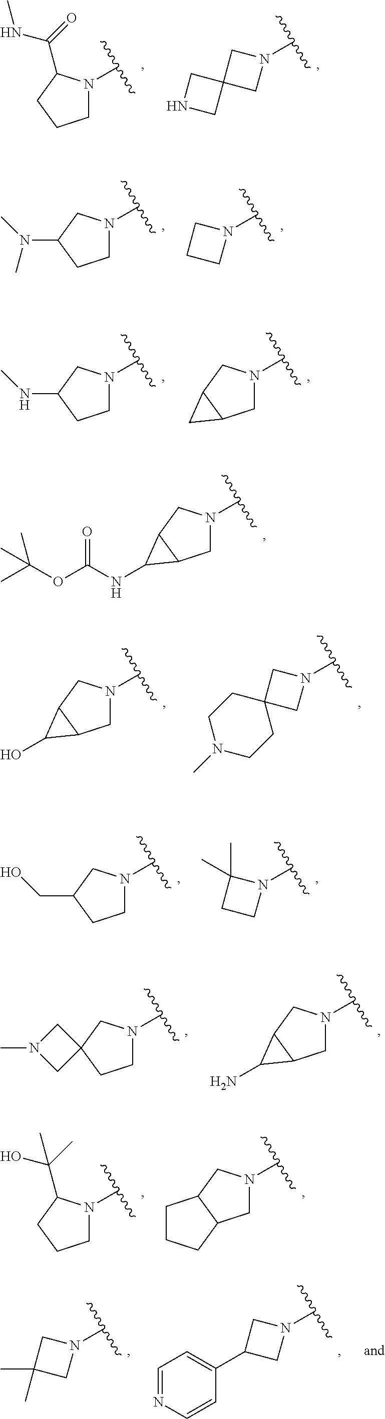

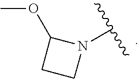 [SMILES: *N1CCC1OC]

62. The compound of any one of claims 1-11 and 58-61, or a pharmaceutically acceptable salt thereof, wherein

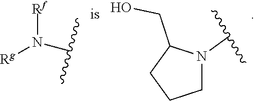

63. The compound of any one of claims 1-11, or a pharmaceutically acceptable salt thereof, wherein R⁴ is a 5- to 10 membered heteroaryl optionally substituted with one or more independently selected C₁-C₆ alkyl substituents.

64. The compound of any one of claims 1-11 and 63, or a pharmaceutically acceptable salt thereof, wherein R⁴ is selected from the group consisting of

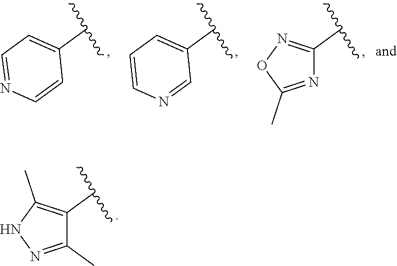

65. The compound of any one of claims 1-11, or a pharmaceutically acceptable salt thereof, wherein R⁴ is Z⁶S(O)₂N(R^{s})—.

66. The compound of any one of claims 1-11 and 65, or a pharmaceutically acceptable salt thereof, wherein R⁴ is

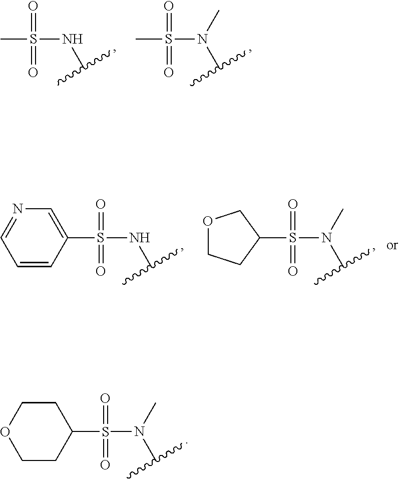

67. The compound of any one of claims 1-11, or a pharmaceutically acceptable salt thereof, wherein R⁴ is Z⁷N(R^{t})S(O)₂—.

68. The compound of any one of claims 1-11 and 67, or a pharmaceutically acceptable salt thereof, wherein R⁴ is —S(O)₂—NH-phenyl.

69. The compound of any one of claims 1-11, or a pharmaceutically acceptable salt thereof, wherein R⁴ is Z⁸—O—(CH₂)_{q}—.

70. The compound of any one of claims 1-11 and 69, or a pharmaceutically acceptable salt thereof, wherein R⁴ is

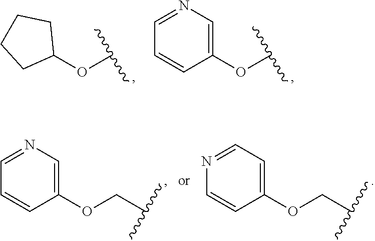

71. The compound of any one of claims 1-4, or a pharmaceutically acceptable salt thereof, wherein p is 0.

72. The compound of any one of claims 1-4 and 71, or a pharmaceutically acceptable salt thereof, wherein the compound is of Formula (II-A) 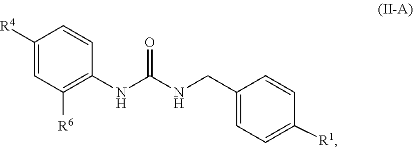
or a pharmaceutically acceptable salt thereof, wherein:
R¹ is halo or methoxy;
R⁴ is
l) 3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents,
m) 3- to 6-membered heterocycloalkyl or heterocycloalkenyl comprising exactly one annular heteroatom, which is an oxygen atom, wherein the 3- to 6-membered heterocycloalkyl or heterocycloalkenyl is optionally substituted with one or more independently selected oxo or —C₁-C₆ alkyl substituents,
n) 3- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents,
o) 5-membered heterocycloalkyl or heterocycloalkenyl comprising exactly two annular heteroatoms, one of which is a nitrogen atom and the other of which is an oxygen atom, wherein the 5-membered heterocycloalkyl or heterocycloalkenyl is optionally substituted with one or more independently selected oxo, —C₁-C₆ alkyl, or —S(O)₂—(C₁-C₆ alkyl) substituents,
p) 6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, —C₁-C₆ alkyl, or —S(O)₂—(C₁-C₆ alkyl) substituents,
q) 5-membered heteroaryl comprising exactly two annular heteroatoms, one of which is a nitrogen atom and the other of which is an oxygen atom, wherein the 5-membered heteroaryl is substituted with exactly one methyl substituent,
r) 5-membered heteroaryl comprising exactly two annular heteroatoms, both of which are nitrogen atoms, wherein the 5-membered heteroaryl is substituted with one or more methyl substituents,
s) 6-membered heteroaryl comprising one or two annular heteroatoms and optionally substituted with one or more methyl substituents, wherein the 6-membered heteroaryl is other than 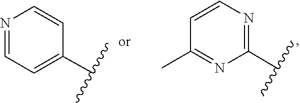
t) Z⁹—S(O)₂—,
u) Z¹⁰—S(O)₂—NH—,
v) Z¹¹—C(O)—NH—,
w) Z¹²—CH₂—O—,
x) Z¹³—O—,
y) Z¹⁴—C(H)(C₁-C₆ alkyl)-NH—C(O)—,
z) 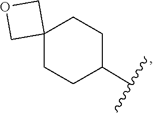 [SMILES: *C1CCC2(CC1)COC2]
or
aa) 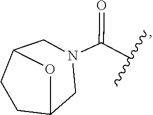
wherein
Z⁹ is selected from the group consisting of cyclopropyl, C₆-C₁₂ aryl, 3- to 10-membered heterocycloalkyl or hetercycloalkenyl optionally substituted with one or more independently selected R^{A} substituents, —NH(C₁-C₆ alkyl), —NH₂ substituted with one or more independently selected R^{B} substituents, and C₁-C₆ alkyl optionally substituted with one or more independently selected R^{C} substituents, provided that Z⁹ is other than 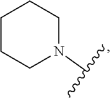 [SMILES: *N1CCCCC1]
unsubstituted methyl, or unsubstituted ethyl, wherein:
R^{A} is —C₁-C₆ alkyl or —CN; and
R^{B} is (i) —C₁-C₆ alkyl-(5- to 10-membered heteroaryl), or (ii) 5- to 10-membered heteroaryl optionally substituted with one or more independently selected C₆-C₁₂ aryl; and
R^{C} is 3- to 8-membered heterocycloalkyl or heterocycloalkenyl;
Z¹⁰ is C₁-C₆ alkyl substituted with one or more independently selected C₆-C₁₂ aryl substituents;
Z¹¹ is selected from the group consisting of C₃-C₁₀ cycloalkyl and C₁-C₆ alkyl substituted with one or more independently selected 3- to 10-membered heterocycloalkyl or hetercycloalkenyl substituents, provided that, when Z¹¹ is cyclopropyl, then R¹ is other than methoxy;
Z¹² is selected from the group consisting of C₆-C₁₂ aryl, 5- to 10-membered heteroaryl, 3- to 10-membered heterocycloalkyl or heterocycloalkenyl, C₁-C₆ alkyl substituted with one or more independently selected 3- to 10-membered heterocycloalkyl or hetercycloalkenyl substituents or 5- to 10-membered heteroaryl substituents, and —C(O)-(3- to 10-membered heterocycloalkyl or heterocycloalkenyl);
Z¹³ is 5- to 10-membered heteroaryl substituted with one or more independently selected —C(O)—NH(C₁-C₆ alkyl) substituents; and
Z¹⁴ is 5- to 10-membered heteroaryl optionally substituted with one or more independently selected C₁-C₆ alkyl substituents; and
R⁶ is hydrogen or halo.

73. The compound of any one of claims 1-4, 71, and 72, or a pharmaceutically acceptable salt thereof, wherein R⁴ is selected from the group consisting of:
3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents,
3- to 6-membered heterocycloalkyl or heterocycloalkenyl comprising exactly one annular heteroatom, which is an oxygen atom, wherein the 3- to 6-membered heterocycloalkyl or heterocycloalkenyl is optionally substituted with one or more independently selected oxo or —C₁-C₆ alkyl substituents,
3- to 6-membered heterocycloalkyl or heterocycloalkenyl substituted with one or more independently selected —S(O)₂—C₁-C₆alkyl substituents and optionally further substituted with one or more independently selected oxo or —C₁-C₆ alkyl substituents,
5-membered heterocycloalkyl or heterocycloalkenyl comprising exactly two annular heteroatoms, one of which is a nitrogen atom and the other of which is an oxygen atom, wherein the 5-membered heterocycloalkyl or heterocycloalkenyl is optionally substituted with one or more independently selected oxo, —C₁-C₆ alkyl, or —S(O)₂—(C₁-C₆ alkyl) substituents, and
6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, —C₁-C₆ alkyl, or —S(O)₂—(C₁-C₆ alkyl) substituents.

74. The compound of any one of claims 1-4 and 71-73, or a pharmaceutically acceptable salt thereof, wherein R⁴ is selected from the group consisting of

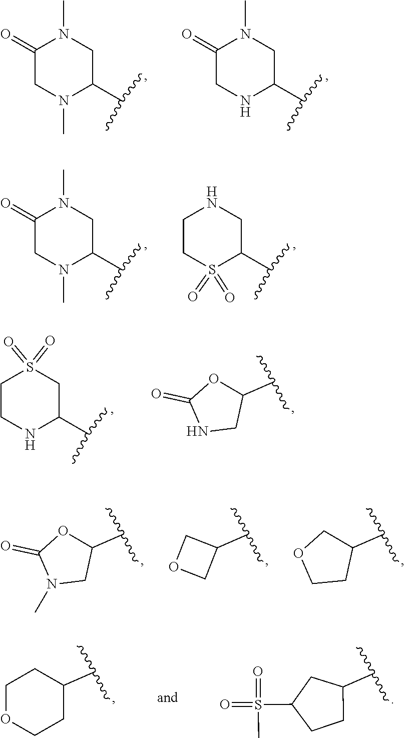

75. The compound of any one of claims 1-4 and 71-73, or a pharmaceutically acceptable salt thereof, wherein R⁴ is
3- to 6-membered heterocycloalkyl or heterocycloalkenyl comprising exactly two annular heteroatoms, both of which are nitrogen atoms, wherein the 3- to 6-membered heterocycloalkyl or heterocycloalkenyl is substituted with one or more independently selected —C₁-C₆ alkyl substituents and is optionally further substituted with one or more oxo substituents, or
6-membered heterocycloalkyl or heterocycloalkenyl comprising exactly two annular heteroatoms, one of which is a sulfur atom and the other of which is a nitrogen atom, wherein the 6-membered heterocycloalkyl or heterocycloalkenyl is optionally substituted with one or more independently selected oxo, —C₁-C₆ alkyl, or —S(O)₂—(C₁-C₆ alkyl) substituents.

76. The compound of any one of claims 1-4, 71-73, and 75, or a pharmaceutically acceptable salt thereof, wherein R⁴ is

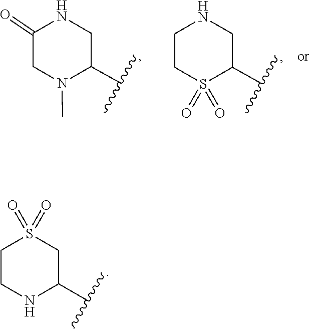

77. The compound of any one of claims 1-4 and 71, or a pharmaceutically acceptable salt thereof, wherein R⁴ is selected from the group consisting of:
5-membered heteroaryl comprising exactly two annular heteroatoms, one of which is a nitrogen atom and the other of which is an oxygen atom, wherein the 5-membered heteroaryl is substituted with exactly one methyl substituent,
5-membered heteroaryl comprising exactly two annular heteroatoms, both of which are nitrogen atoms, wherein the 5-membered heteroaryl is substituted with one or more methyl substituents, and
6-membered heteroaryl comprising one or two annular heteroatoms and optionally substituted with one or more methyl substituents, wherein the 6-membered heteroaryl is other than

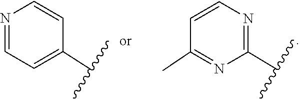

78. The compound of any one of claims 1-4, 71, and 77, or a pharmaceutically acceptable salt thereof, wherein R⁴ is selected from the group consisting of

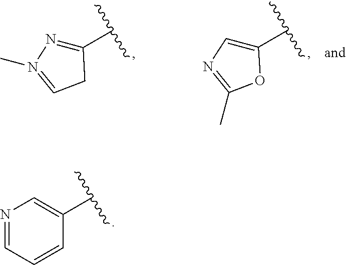

79. The compound of any one of claims 1-4 and 71, or a pharmaceutically acceptable salt thereof, wherein R⁴ is Z⁹—S(O)₂—.

80. The compound of any one of claims 1-4, 71, and 79, or a pharmaceutically acceptable salt thereof, wherein Z⁹ is selected from the group consisting of

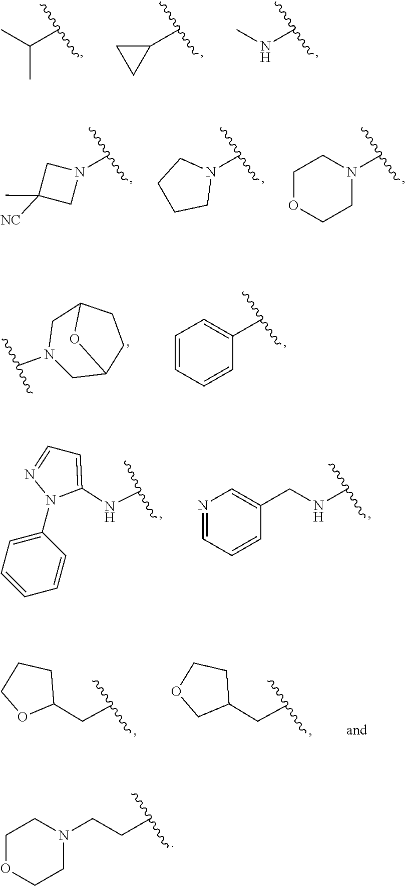

81. The compound of any one of claims 1-4 and 71, or a pharmaceutically acceptable salt thereof, wherein R⁴ is Z¹⁰—S(O)₂—NH—.

82. The compound of any one of claims 1-4, 71, and 81, or a pharmaceutically acceptable salt thereof, wherein Z¹⁰ is

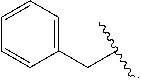 [SMILES: *Cc1ccccc1]

83. The compound of any one of claims 1-4 and 71, or a pharmaceutically acceptable salt thereof, wherein R⁴ is Z¹¹—C(O)—NH—.

84. The compound of any one of claims 1-4, 71, and 83, or a pharmaceutically acceptable salt thereof, wherein Z¹¹ is

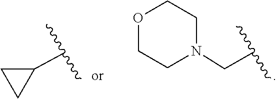

85. The compound of any one of claims 1-4 and 71, or a pharmaceutically acceptable salt thereof, wherein R⁴ is Z¹²—CH₂—O—.

86. The compound of any one of claims 1-4, 71, and 85, or a pharmaceutically acceptable salt thereof, wherein Z¹² is selected from the group consisting of

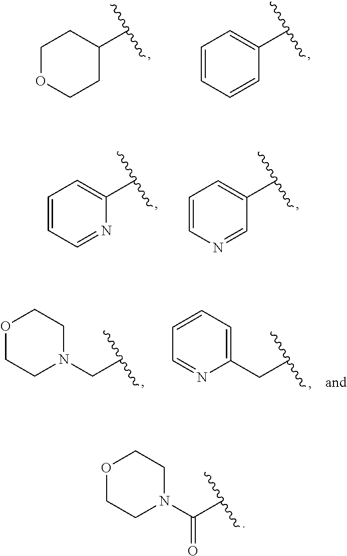

87. The compound of any one of claims 1-4 and 71, or a pharmaceutically acceptable salt thereof, wherein R⁴ is Z¹³—O—.

88. The compound of any one of claims 1-4, 71, and 87, or a pharmaceutically acceptable salt thereof, wherein Z¹³ is

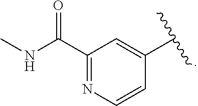 [SMILES: *c1ccnc(C(=O)NC)c1]

89. The compound of any one of claims 1-4 and 71, or a pharmaceutically acceptable salt thereof, wherein R⁴ is Z¹⁴—C(H)(C₁-C₆ alkyl)-NH—C(O)—.

90. The compound of any one of claims 1-4, 71, and 89, or a pharmaceutically acceptable salt thereof, wherein R⁴ is

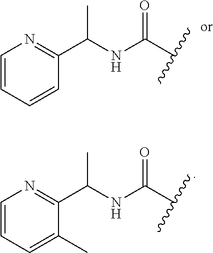 [SMILES: *C(=O)NC(C)c1ccccn1 ; *C(=O)NC(C)c1ncccc1C]

91. The compound of any one of claims 1-4 and 71, or a pharmaceutically acceptable salt thereof, wherein R⁴ is

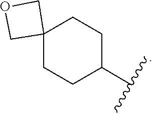 [SMILES: *C1CCC2(CC1)COC2]

92. The compound of any one of claims 1-4 and 71, or a pharmaceutically acceptable salt thereof, wherein R⁴ is

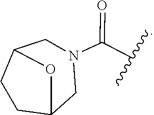 [SMILES: *C(=O)N1CC2CCC(C1)O2]

93. A compound selected from the group consisting of compounds of Table 1, or a pharmaceutically acceptable salt thereof.

94. A pharmaceutical composition comprising a compound according to any one of claims 1-93, or a pharmaceutically acceptable salt thereof, and a pharmaceutically acceptable excipient.

95. A method of treating a disease or condition mediated by NAMPT activity in a subject in need thereof, comprising administering to the subject a compound of any one of claims 1-93, or a pharmaceutically acceptable salt thereof, or a pharmaceutical composition of claim 94.

96. The method of claim 95, wherein the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder.

97. The method of claim 95, wherein the disease or condition is selected from the group consisting of obesity, atherosclerosis, insulin resistance, type 2 diabetes, cardiovascular disease, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.
